SEQUENCE LISTING (1 sequence)

SEQ ID NO: 1 - RNA, 20 nt, synthetic construct
  source 1..20: mol_type = other RNA
cgattagggtcagaccgcag

(19) United States
(12) Patent Application Publication
(10) Pub. No.: US 2025/0235543 A1
(43) Pub. Date: Jul. 24, 2025

(54) MODIFIED PROTEINS AND PROTEIN DEGRADERS

(71) Applicant: Cullgen (Shanghai), Inc., Shanghai (CN)

(72) Inventors: Jing LIU, San Diego, CA (US); Michael Bruno PLEWE, San Diego, CA (US); Xiaoran HAN, Shanghai (CN); Chengwei ZHANG, Shanghai (CN); Ting YANG, Shanghai (CN); Liqun CHEN, Shanghai (CN); Matthew Randolph LEE, San Diego, CA (US); Jing ZHOU, Shanghai (CN); Jie DING, Shanghai (CN); Jialiang WANG, San Diego, CA (US)

(21) Appl. No.: 18/700,898

(22) PCT Filed: Oct. 13, 2022
(86) PCT No.: PCT/CN2022/125080

§ 371 Date: Apr. 12, 2024

(30) Foreign Application Priority Data
Oct. 14, 2021 (WO) PCT/CN2021/123848
Nov. 26, 2021 (WO) PCT/CN2021/133363

(51) Int. Cl. A61K 47/55; A61P 35/00

(52) U.S. Cl. CPC A61K 47/55; A61P 35/00

(57) ABSTRACT

Provided herein are compounds, pharmaceutical compositions, and methods for binding or degrading target proteins. Further provided herein are bifunctional compounds having a DNA damage-binding protein 1 (DDB 1) binding moiety, a linker, and a target binding moiety.

### CROSS-REFERENCE

This application claims the benefit of PCT Application No. PCT/CN2021/123848, filed Oct. 14, 2021, and PCT Application No. PCT/CN2021/133363, filed Nov. 26, 2021, which applications are incorporated herein by reference in their entireties.

### SEQUENCE LISTING

The instant application contains a Sequence Listing which has been submitted electronically in XML file format and is hereby incorporated by reference in its entirety. Said XML copy is entitled 54922_715_603_SL.xml, was created on Oct. 5, 2022 and is 1727 bytes in size.

### BACKGROUND

Progression through the cell cycle is part of the development of a single-celled fertilized egg to into a mature organism. Such progression involves a series of cellular events, including DNA replication and cell division into daughter cells. Cell proliferation is controlled at the G1 phase of the cell cycle, which is further regulated in mammalian cells primarily by CDK4 and its closely related paralog, CDK6. CDK4/6 by themselves are catalytically inactive and are activated by the binding of cyclin D proteins. Human cells express three cyclin D proteins—D1, D2, and D3, which are expressed at low levels in non-dividing cells. Various mitogenic signals can transcriptionally activate cyclin D protein, leading to CDK4/6 activation. Activated CDK4/6 catalyze the phosphorylation of retinoblastoma (RB) proteins RB1, p107 (RBL1), and p130 (RBL2). RB proteins, in their hypophosphorylated state, bind to and inhibit the function of transcription factors in the E2F family. Phosphorylation of RB proteins by CDK4/6 dissociates them from E2F and allows E2F to activate the expression of multiple genes involved in DNA replication. CDK4/6 inhibitors, such as INK4, negatively regulate CDK4/6 and cell proliferation in a RB— dependent manner. INK4, cyclin D, CDK4/6, and RB are part of a pathway that controls the G1-to-S transition.

The cell cycle lies at the heart of many cancers. Dysregulation of the INK4-cyclinD-CDK4/6-RB pathway is an important first for cell transformation, and the initiation of most cancers. Cancer genomic studies have further validated the importance of the INK4-cyclin D-CDK4/6-RB pathway in cancer development: all genes on this pathway are frequently mutated in various types of cancer, including breast cancer, glioblastoma (GBM), ovarian cancer, lung cancer, esophageal squamous cell carcinoma (ESCC), liver cancer, bladder cancer, head and neck squamous cell carcinoma (HNSCC), skin cutaneous melanoma (SKCM).

Among the genes on the INK4-cyclin D-CDK4/6-RB pathway, cyclin D represents a high-value cancer target. As the first identified cell cycle oncogene, cyclin D is frequently amplified in a wide range of human cancers by the mechanism of genomic amplification or overexpression, including 23-57% ESCC, 26-39% HNSCC, 5-30% NSCLC, 25% pancreatic cancer, 15-20% breast cancer, 26% endometrial cancer. In addition to its function as CDK4/6 activator, cyclin D has CDK4/6- and RB-independent functions. For example, cyclin D interacts with transcriptional factors and regulates their activities. Moreover, analysis of cyclin D interactors through a proteomic screen revealed its function in DNA repair. Another study demonstrated the kinase-independent role of cyclin D in chromosomal instability. Cyclin D was recently identified as the top cancer therapeutic target by the functional cancer dependency map (DepMap) project. The lack of a functional active site, however, has rendered cyclin D as previously undruggable.

Three CDK4/6 inhibitors, palbociclib, ribociclib, and abemaciclib, have been approved for patients with hormone receptor-positive (HR+), human epidermal growth factor receptor 2-negative (HER2−) metastatic breast cancer, in combination with endocrine therapy (ET), such as estrogen receptor (ER) inhibitors and aromatase inhibitors (AIs). Abemaciclib is also approved as monotherapy in men and women with disease progression following ET and prior chemotherapy in the metastatic setting. Each agent has shown to significantly improve progression-free survival (PFS) when combined to endocrine therapy. However, between 33% to 70% of patients developed acquired resistance after 2 to 3 years of treatment with CDK4/6 inhibitors.

Most resistance to CDK4/6 inhibitors is not linked to active site mutations, as seen with other kinase inhibitors, that might be overcome by developing next generation inhibitors. Instead, mutation of genes upstream of cyclin D, such as RTK, RAS, AKT, YAP, appears to be a common theme and is associated with upregulated cyclin D expression. Therefore, suppression of cyclin D could potentially achieve higher potency than CDK4/6 inhibitor alone, overcome resistance to CDK4/6 inhibitors and target CDK4/6-independent oncogenic function of cyclin D.

A need exists in the medicinal arts for compounds and methods for selective degradation of target proteins, including cyclin D.

### SUMMARY

Disclosed herein are heterobifunctional compounds and compositions comprising a DDB1 (damaged DNA binding protein 1) E3 ligase binding moiety linked to a target protein binding moiety through a bivalent linker, and methods of making and using such compounds and compositions.

Disclosed herein, in one aspect is a heterobifunctional compound of Formula (I), or a pharmaceutically acceptable salt or solvate thereof:

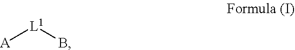

wherein,
A is a target protein binding moiety;
L¹ is a linker; and
B is a DDB1 binding moiety having the structure of Formula (II):

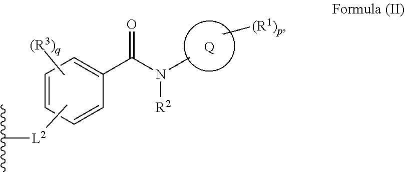

wherein,
ring Q is phenyl or a 5 or 6-membered monocyclic heteroaryl;
L² is a bond, —O—, —NR^{4A}—, —NR^{4B}—C(═O)—, —NR^{4B}—C(═O)—(C₁-C₃alkylene)-NR^{4A}—, —NR^{4B}—C(═O)—(C₁-C₃alkylene)-O—, —(C₁-C₃alkylene)-NR^{4B}—C(═O)—, —C(═O)NR^{4A}—, —C₁-C₃alkylene-, —C₂-C₃ alkenylene-, —C₂-C₃alkynylene-, C₃-C₈ cycloalkylene, or C₂-C₈ heterocyclene;
each R¹ is independently hydrogen, halogen, —CN, NO₂, —OR^{4A}, —NR^{4A}R^{4B}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl or heteroaryl, or
two R¹, together with the atom(s) to which they are connected, optionally form C₃-C₁₃ cycloalkyl, C₂-C₁₂ heterocyclyl, aryl, or heteroaryl;
R² is hydrogen, C₁-C₆ alkyl, C₃-C₈ cycloalkyl, OH, or O—C₁-C₄ alkyl;
each R³ is independently hydrogen, halogen, —CN, —NO₂, —OR^{4A}, —NR^{4A}R^{4B}, —C(═O)R^{4A}, —C(═O)OR^{4A}, —C(═O)NR^{4B}R^{4A}, —OC(═O)R^{4A}, —N(R^{4A})C(═O)R^{4B}, C₁-C₆ alkyl, C₁-C₆ haloalkyl, C₁-C₆ heteroalkyl, C₃-C₈ cycloalkyl, C₂-C₈ heterocyclyl, aryl, or heteroaryl, or
two R³, together with the atom(s) to which they are connected, optionally form C₃-C₁₃ cycloalkyl, C₂-C₁₂ heterocyclyl, aryl, or heteroaryl;
each R^{4A} and R^{4B} is independently hydrogen, C₁-C₆ alkyl, C₂-C₆ alkenyl, C₂-C₆ alkynyl, C₁-C₆ haloalkyl, C₁-C₆ heteroalkyl, C₃-C₈ cycloalkyl, C₂-C₈ heterocyclyl, aryl, or heteroaryl, or
R^{4A} and R^{4B}, together with the atom(s) to which they are connected, optionally form C₂-C₁₂ heterocyclyl;
p is 1, 2 or 3; and
q is 1, 2 or 3.

In some embodiments, ring Q is a 5-membered monocyclic heteroaryl. In some embodiments, the 5-membered monocyclic heteroaryl is pyrrolyl, furanyl, imidazolyl, pyrazolyl, oxazolyl, isoxazolyl, thienyl, thiazolyl, isothiazolyl, triazolyl, oxadiazolyl, thiadiazolyl, or tetrazolyl.

In some embodiments, the DDB1 binding moiety of Formula (II) has the structure of Formula (III-1) or (III-2), or a pharmaceutically acceptable salt or solvate thereof:

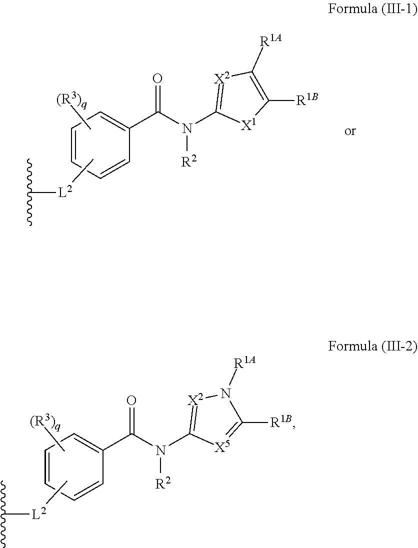

wherein,
X¹ is O, S, or NR⁵;
X² and X⁵ are independently N or CH;
R⁵ is hydrogen, C₁-C₆ alkyl, C₁-C₆ haloalkyl, C₁-C₆ heteroalkyl, C₃-C₈ cycloalkyl, or C₂-C₈ heterocyclyl; and
R^{1A} and R^{1B} are independently selected from hydrogen, halogen, CN, —NO₂, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ heteroalkyl, C₁-C₆ haloalkyl, C₃-C₈ cycloalkyl, C₂-C₈ heterocyclyl, aryl or heteroaryl, or
R^{1A} and R^{1B}, together with the atom(s) to which they are connected, optionally form C₃-C₁₃ cycloalkyl, C₂-C₁₂ heterocyclyl, aryl, or heteroaryl.

In some embodiments, X¹ is O or S; and X² is N. In some embodiments, R² is H. In some embodiments, X⁵ is CH.

In some embodiments, R^{1A} is selected from hydrogen, halogen, NO₂, —OCH₃, —C(═O)CH₃, —C(═O)OCH₃, —C(═O)NH₂, —C(═O)NHCH₃, —C(═O)N(CH₃)₂, —CH₃, —CF₃, —CH₂CH₃, —CH(CH₃)₂, —C(CH₃)₃, cyclopropyl, cyclobutyl, cyclopentyl, cyclohexyl, or phenyl. In some embodiments, R^{1A} is selected from hydrogen, halogen, —OCH₃, —C(═O)CH₃, —C(═O)OCH₃, —CH₃, —CF₃, —CH₂CH₃, —CH(CH₃)₂, —C(CH₃)₃, cyclopropyl, cyclobutyl, cyclopentyl, cyclohexyl, or phenyl. In some embodiments, R^{1B} is selected from hydrogen, halogen, NO₂, —OCH₃, —C(═O)CH₃, —C(═O)OCH₃, —C(═O)NH₂, —C(═O)NHCH₃, —C(═O)N(CH₃)₂, —CF₃, or phenyl. In some embodiments, R^{1B} is selected from hydrogen, halogen, —OCH₃, —C(═O)CH₃, —C(═O)OCH₃, —CF₃, or phenyl. In some embodiments, R^{1B} is selected from —CH₃, —CH(CH₃)₂, —C(CH₃)₃, cyclopropyl, cyclobutyl, cyclopentyl, or cyclohexyl.

In some embodiments, ring Q is a phenyl or 6-membered monocyclic heteroaryl. In some embodiments, the 6-membered monocyclic heteroaryl is pyridinyl, pyridazinyl, pyrazinyl, pyrimidinyl, or triazinyl.

In some embodiments, the DDB1 binding moiety of Formula (II) has the structure of Formula (V-1), or a pharmaceutically acceptable salt or solvate thereof:

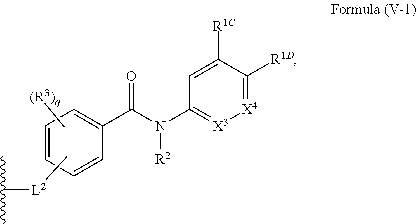

wherein,
X³ is N or CH;
X⁴ is CR^{1E} or N; and
each of R^{1C}, R^{1D}, and R^{1E} is independently selected from hydrogen, halogen, CN, —NO₂, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl or heteroaryl, or
R^{1C} and R^{1D}, or R^{1D} and R^{1E}, together with the atom(s) to which they are connected, optionally form C₃-C₁₃ cycloalkyl, C₂-C₁₂ heterocyclyl, aryl, or heteroaryl.

In some embodiments, R² is hydrogen. In some embodiments, X³ is N. In some embodiments, X³ is CH. In some embodiments, R^{1C} and R^{1E} are each hydrogen; and R^{1D} is hydrogen, halogen, —NO₂, CN, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl, or heteroaryl. In some embodiments, R^{1C} and R^{1E} are each hydrogen; and R^{1D} is hydrogen, halogen, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, 4 to 7-membered heterocycloalkyl, aryl, or heteroaryl.

In some embodiments, X³ and X⁴ are N; R^{1C} is hydrogen; and R^{1D} is hydrogen, halogen, —NO₂, CN, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl, or heteroaryl. In some embodiments, X³ and X⁴ are N; R^{1C} is hydrogen; and R^{1D} is hydrogen, halogen, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, 4 to 7-membered heterocycloalkyl, aryl, or heteroaryl. In some embodiments, X³ and X⁴ are N; R^{1C} is hydrogen; and R^{1D} is —OR^{4A}, —NR^{4B}R^{4A}, C₁-C₆ alkyl, C₁-C₆ haloalkyl, C₃-C₈ cycloalkyl, C₂-C₈ heterocyclyl. In some embodiments, X³ and X⁴ are N; R^{1C} is hydrogen; and R^{1D} is —NR^{4B}R^{4A}. In some embodiments, X³ and X⁴ are N; R^{1C} is hydrogen; and R^{1D} is —N(CH₃)₂.

In some embodiments, X³ is N; X⁴ is CR^{1E}; R^{1C} is hydrogen; and R^{1D} and R^{1E} are independently selected from hydrogen, halogen, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, 4 to 7-membered heterocycloalkyl, aryl, or heteroaryl. In some embodiments, X³ is N; X⁴ is CR^{1E}; R^{1C} is hydrogen; and R^{1D} and R^{1E} are independently selected from hydrogen, halogen, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, 4 to 7-membered heterocycloalkyl, aryl, or heteroaryl. In some embodiments, X³ is N; X⁴ is CR^{1E}; R^{1C} is hydrogen; and R^{1D} and R^{1E} are independently selected from hydrogen, halogen, —OR^{4A}, —NR^{4B}R^{4A}, C₁-C₆ alkyl, C₁-C₆ haloalkyl, C₁-C₆ heteroalkyl, C₃-C₈ cycloalkyl, or 4 to 7-membered heterocycloalkyl.

In some embodiments, X³ is N; X⁴ is CR^{1E}; R^{1C} is hydrogen; and R^{1D} and R^{1E}, together with the atom(s) to which they connected, form C₃-C₁₃ cycloalkyl, C₂-C₁₂ heterocyclyl, aryl, or heteroaryl.

In some embodiments, each R³ is independently halogen, C₁-C₆ alkyl, C₁-C₆ haloalkyl, C₁-C₆ heteroalkyl, C₁-C₆ alkoxy, C₁-C₆ alkylamino, C₃-C₆ cycloalkoxy, C₃-C₆ cycloalkylamino, C₃-C₈ cycloalkyl, or C₂-C₈ heterocyclyl. In some embodiments, each R³ is independently halogen, CN, C₁-C₆ alkyl, C₁-C₆ haloalkyl, C₁-C₆ heteroalkyl, C₁-C₆ alkoxy, C₁-C₆ alkylamino, C₁-C₆ alkylamido, C₃-C₆ cycloalkoxy, C₃-C₆ cycloalkylamino, C₃-C₆ cycloalkylamido, C₃-C₈ cycloalkyl, or C₂-C₈ heterocyclyl. In some embodiments, R³ is halogen. In some embodiments, R³ is F or Cl. In some embodiments, R³ is C₁-C₆ haloalkyl. In some embodiments, R³ is CHF₂ or CF₃. In some embodiments, R³ is CN. In some embodiments, R³ is C₁-C₆ alkylamino. In some embodiments, R³ is C₁-C₆ alkyl. In some embodiments, R³ is CH₃. In some embodiments, R³ is CH₃, CH₂CH₃, CH(CH₃)₂, C(CH₃)₃, or cyclopropyl.

In some embodiments, two R³, together with the atom(s) to which they are connected, form C₃-C₁₃ cycloalkyl, C₂-C₁₂ heterocyclyl, aryl, or heteroaryl. In some embodiments, two R³, together with the atom(s) to which they are connected, form C₅-C₆ cycloalkyl, 5-6 membered heterocyclyl, phenyl, or 5-6 membered heteroaryl. In some embodiments, two R³, together with the atom(s) to which they are connected, form cyclopentyl, cyclohexyl, pyrrole, pyrazole, or imidazole.

In some embodiments, p is 1 or 2. In some embodiments, L² is a bond. In some embodiments, L² is —C(═O)NR^{4B}—, —NR^{4A}—(C₁-C₃alkylene)-C(═O)NR^{4B}—, or —O—(C₁-C₃ alkylene)-C(═O)NR^{4B}—. In some embodiments, L² is —C(═O)NH—, —NH—(CH₂)—C(═O)NH—, or —O—(CH₂)—C(═O)NH—. In some embodiments, L² is —NR^{4A}— or —O—. In some such embodiments, L² is —NH—. In some such embodiments, L² is —O—.

In some embodiments, linker L¹ is a divalent moiety having the structure of Formula (L), or a pharmaceutically acceptable salt or solvate thereof:

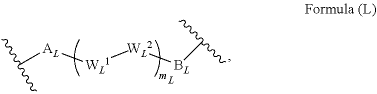

wherein,
A_{L}, W_{L}¹, W_{L}², and B_{L}, at each occurrence, is a bivalent moiety independently selected from the group consisting of a bond (i.e., the group is absent), R_{L}^{a}—R_{L}^{b}, R_{L}^{a}COR_{L}^{b}, R_{L}^{a}C(O)OR_{L}^{b}, R_{L}^{a}C(O)N(R_{L}¹)R_{L}^{b}, R_{L}^{a}C(S)N(R_{L}¹)R_{L}^{b}, R_{L}^{a}OR_{L}^{b}, R_{L}^{a}SR_{L}^{b}, R_{L}^{a}SOR_{L}^{b}, R_{L}^{a}SO₂R_{L}^{b}, R_{L}^{a}SO₂N(R_{L}¹)R_{L}^{b}, R_{L}^{a}N(R_{L}¹)R_{L}^{b}, R_{L}^{a}N(R_{L}¹)COR_{L}^{b}, R_{L}^{a}N(R_{L}¹)CON(R_{L}²)R_{L}^{b}, R_{L}^{a}N(R_{L}¹)C(S)R_{L}^{b}, optionally substituted C₁-C₈ alkylene, optionally substituted C₂-C₈ alkenylene, optionally substituted C₂-C₈ alkynylene, optionally substituted 1-8 membered heteroalkylene, optionally substituted 2-8 membered heteroalkenylene, optionally substituted 2-8 membered heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ cycloalkylene, optionally substituted 3-13 membered heterocyclene, optionally substituted arylene, and optionally substituted heteroarylene, wherein
each R_{L}^{a} and R_{L}^{b} is independently a bond (i.e., the group is absent), R_{L}^{r}, optionally substituted (C₁-C₈ alkylene)-R_{L}^{r}, optionally substituted R_{L}^{r}—(C₁-C₈ alkylene), optionally substituted (C₁-C₈ alkylene)-R_{L}^{r}—(C₁-C₈ alkylene), or a bivalent moiety comprising of optionally substituted C₁-C₈ alkylene, optionally substituted C₂-C₈ alkenylene, optionally substituted C₂-C₈ alkynylene, optionally substituted 1-8 membered heteroalkylene, optionally substituted 2-8 membered heteroalkenylene, optionally substituted 2-8 membered heteroalkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ cycloalkylene, optionally substituted 3-13 membered heterocyclene, optionally substituted arylene, or optionally substituted heteroarylene;
each R_{L}^{r} is independently selected from optionally substituted C₃-C₁₀ cycloalkylene, optionally substituted 3-10 membered heterocyclene, optionally substituted arylene, and optionally substituted heteroarylene;
each R_{L}¹ and R_{L}² are independently selected from the group consisting of hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ cycloalkyl, optionally substituted 3-10 membered heterocyclyl, optionally substituted aryl, and optionally substituted heteroaryl; or
R_{L}^{a} and R_{L}^{b}, R_{L}¹ and R_{L}², R_{L}^{a} and R_{L}¹, R_{L}^{a} and R_{L}², R_{L}^{b} and R_{L}¹, or R_{L}^{b} and R_{L}² together with the atom(s) to which they are attached optionally form a C₃-C₂₀ carbocyclyl or 3-20 membered heterocyclyl ring; and
m_{L} is an integer from 1 to 15.

In some embodiments, A_{L} is a bond, —C(═O)—, —C(═O)NH—, —NH—, —NH—C(═O)—, —O—, —(C₁-C₈ alkylene)-C(═O)NH—, —(C₁-C₈ alkylene)-C(═O)—, —(C₁-C₈ alkylene)NH—, —(C₁-C₈ alkylene)-NH—C(═O)—, —(C₁-C₈ alkylene)-O—, —C₁-C₈ alkylene-, or —C₂-C₈ alkynylene-. In some embodiments, B_{L} is a bond, —C(═O)—, —C(═O)NH—, —NH—, —NH—C(═O)—, —O—, —(C₁-C₈ alkylene)-, —NH—(C₁-C₈ alkylene)-, —O—(C₁-C₈ alkylene)-, —C(═O)—(C₁-C₈ alkylene)-, —C(═O)NH—(C₁-C₈ alkylene)-, —NH—C(═O)—(C₁-C₈ alkylene)-, or —C₂-C₈ alkynylene-. In some embodiments, each W_{L}¹ is independently R_{L}^{r} or C₁-C₃ alkylene; and each W_{L}² is independently a bond, O, or NH. In some embodiments, each W_{L}¹ is independently a bond, O, or NH; and each W_{L}² is independently R_{L}^{r}, or C₁-C₃ alkylene. In some embodiments, each W_{L}¹ is independently C₁-C₃ alkylene; and each W_{L}² is independently a bond or O. In some embodiments, each W_{L}¹ is independently a bond or O; and each W_{L}² is independently C₁-C₃ alkylene. In some embodiments, each —W_{L}¹—W_{L}²— is independently —CH₂CH₂O—, or —CH₂—. In some embodiments, m_{L} is selected from 1-10.

In some embodiments, the linker L¹ is —(CH₂)ₚ₁C(═O)NH(CH₂CH₂O)ₚ₂—(CH₂)ₚ₃—, —(CH₂)ₚ₁C(═O)NH(CH₂)ₚ₂—, —(CH₂)ₚ₁NHC(═O)—(CH₂CH₂O)ₚ₂—(CH₂)ₚ₃—, —(CH₂)ₚ₁NHC(═O)—(CH₂)ₚ₂—, —(CH₂)ₚ₁C(═O)—(CH₂CH₂O)ₚ₂—(CH₂)ₚ₃—, —(CH₂)ₚ₁C(═O)—(CH₂)ₚ₂—, —(CH₂)ₚ₁NH(CH₂CH₂O)ₚ₂—(CH₂)ₚ₃—, —(CH₂)ₚ₁NH(CH₂)ₚ₂—, —(CH₂CH₂O)ₚ₂—(CH₂)ₚ₃—, or —(CH₂)ₚ₂—; wherein p1 is an integer from 0 to 8; p2 is an integer from 1 to 15; and p3 is an integer from 0 to 8.

In some embodiments, A is a target protein binding moiety comprising a cyclin-dependent kinase 4 (CDK4) binding moiety or a cyclin-dependent kinase 6 (CDK6) binding moiety.

In some embodiments, the target protein binding moiety has the structure of Formula (A), or a pharmaceutically acceptable salt or solvate thereof:

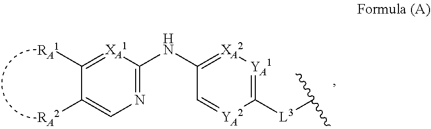

wherein,
X_{A}¹, X_{A}², Y_{A}¹, and Y_{A}² are each independently CR_{A}⁴ or N;
R_{A}¹ is NR_{A}⁵R_{A}⁶, N(R_{A}⁵)C(O)R_{A}⁶, aryl, or heteroaryl;
R_{A}² is hydrogen, halogen, CN, NO₂, C₁-C₈ alkyl, C₁-C₈ haloalkyl, C₁-C₈ alkoxy, C₁-C₈ heteroalkyl, C₃-C₈ cycloalkyl, or C₂-C₈ heterocyclyl, or
R_{A}¹ and R_{A}², together with the atom(s) to which they are attached optionally form an optionally substituted cycloalkyl, heterocyclyl, aryl or heteroaryl;
L³ is a divalent group selected from —R_{A}^{3A}—R_{A}^{3B}-, wherein R_{A}^{3A} and R_{A}^{3B} are each independently a bond (i.e., the group is absent), —O—, —S—, —NR_{A}⁷—, —C(═O)—, —C(═O)NR_{A}⁷—, —S(═O)—, —S(═O)NR_{A}⁷—, —S(═O)₂—, —S(═O)₂NR_{A}⁷—, C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₃ cycloalkylene, C₂-C₁₂ heterocyclene, arylene, or heteroarylene;
each R_{A}⁴ is independently selected from hydrogen, halogen, CN, NO₂, NR_{A}⁸R_{A}⁹, —C(═O)R_{A}¹⁰, —C(═O)OR_{A}¹⁰, —C(═O)NR_{A}⁸R_{A}⁹, —NR_{A}⁸C(═O)R_{A}¹⁰, C₁-C₈ alkyl, C₁-C₈ haloalkyl, C₁-C₈ alkoxy, C₁-C₈ alkoxyalkyl, C₁-C₈ heteroalkyl, C₃-C₈ cycloalkyl, C₂-C₈ heterocyclyl, aryl, or heteroaryl;
R_{A}⁵ and R_{A}⁶ are independently selected from hydrogen, C₁-C₈ alkyl, C₁-C₈ haloalkyl, C₁-C₈ alkoxyalkyl, C₁-C₈ heteroalkyl, C₃-C₈ cycloalkyl, C₂-C₈ heterocyclyl, aryl, or heteroaryl, or
R_{A}⁵ and R_{A}⁶ together with the atom(s) to which they are connected optionally form a 3-20 membered heterocyclyl ring; and
R_{A}⁷, R_{A}⁸, R_{A}⁹ and R_{A}¹⁰ are each independently selected from hydrogen, C₁-C₈ alkyl, C₁-C₈ haloalkyl, C₁-C₈ alkoxyalkyl, C₁-C₈ heteroalkyl, C₃-C₈ cycloalkyl, C₂-C₈ heterocyclyl, aryl, or heteroaryl, or
R_{A}⁸ and R_{A}⁹ together with the atom(s) to which they are connected optionally form a 3-20 membered heterocyclyl ring.

In some embodiments, the target protein binding moiety of Formula (A) has the structure of Formula (A1), (A2), or (A3), or a pharmaceutically acceptable salt or solvate thereof:

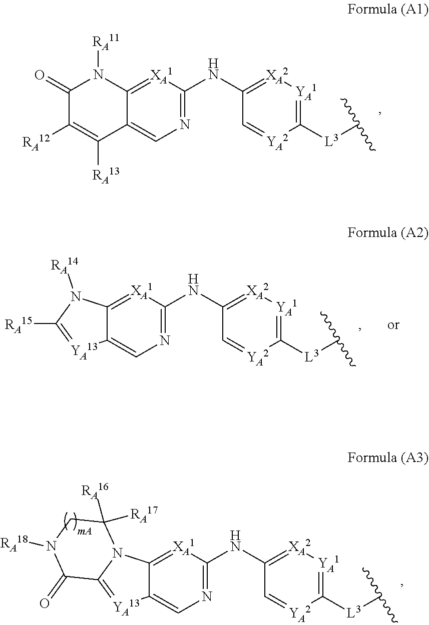

wherein
Y_{A}³ is CR_{A}¹⁹ or N;
R_{A}¹¹, R_{A}¹⁴ and R_{A}¹⁸ are each independently selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, or C₂-C₈ heterocyclyl, aryl, or heteroaryl;
R_{A}¹² and R_{A}¹⁵ are each independently selected from R_{A}²⁰, COR_{A}²⁰, CO₂R_{A}²⁰, or CONR_{A}²⁰R_{A}²¹, wherein R_{A}²⁰ and R_{A}²¹ are independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or C₂-C₈ heterocyclyl, or R_{A}²⁰ and R_{A}²¹, together with the atom(s) to which they are connected optionally form a 3-20 membered heterocyclyl ring;
R_{A}¹³ is selected from hydrogen, halogen, C₁-C₈ alkyl, C₁-C₈ haloalkyl, C₁-C₈ alkoxy, C₁-C₈ alkylamino, C₁-C₈ heteroalkyl, C₃-C₈ cycloalkyl, or C₂-C₈ heterocyclyl;
R_{A}¹⁶ and R_{A}¹⁷ are each independently selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, or C₂-C₈ heterocyclyl, aryl, or heteroaryl, or
R_{A}¹⁶ and R_{A}¹⁷, together with the atom(s) to which they are connected optionally form 3-8 membered cycloalkyl, or 3-8 membered heterocyclyl;
R_{A}¹⁹ are independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or C₂-C₈ heterocyclyl; and
m_{A} is 0, 1, or 2.

In some embodiments, the target protein binding moiety of Formula (A) has the structure of Formula (A4), or a pharmaceutically acceptable salt or solvate thereof:

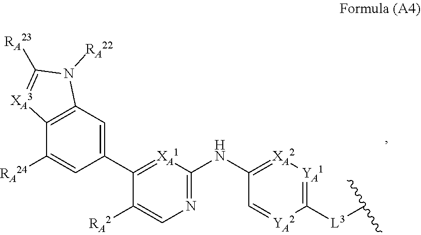

wherein
X_{A}³ is CR_{A}²⁵ or N;
R_{A}²² is selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, C₂-C₈ heterocyclyl, aryl, or heteroaryl; and
R_{A}²³, R_{A}²⁴ and R_{A}²⁵ are each independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or C₂-C₈ heterocyclyl.

In some embodiments, X_{A}¹, X_{A}², and X_{A}³ are each N. In some embodiments, Y_{A}¹, Y_{A}², and Y_{A}³ are each CH.

In some embodiments, m_{A} is 1. In some embodiments, R_{A}¹ is selected from aryl, or heteroaryl. In some embodiments, R_{A}², R_{A}⁴, R_{A}¹³, R_{A}¹⁹, R_{A}²³, and R_{A}²⁴ are each independently selected from hydrogen, halogen, C₁-C₃ alkyl, or C₃-C₆ cycloalkyl.

In some embodiments, R_{A}², R_{A}⁴, R_{A}¹³, R_{A}¹⁹, R_{A}²³, and R_{A}²⁴ are each independently selected from hydrogen, F, Cl, CH₃, CH₂CH₃, CH(CH₃)₂, CF₃, CH₂F, CHF₂, cyclopropyl, or cyclobutyl. In some embodiments, R_{A}¹¹ and R_{A}¹⁴ are each independently selected from hydrogen, C₁-C₈ alkyl, C₃-C₈ cycloalkyl, or C₂-C₈ heterocyclyl. In some embodiments, R_{A}¹¹ and R_{A}¹⁴ are each independently selected from C₁-C₈ alkyl, or C₃-C₈ cycloalkyl. In some embodiments, R_{A}¹² and R_{A}¹⁵ are each independently selected from R_{A}²⁰, COR_{A}²⁰, or CONR_{A}²⁰R_{A}²¹, wherein R_{A}²⁰ and R_{A}²¹ are each independently selected from C₁-C₈ alkyl, C₃-C₈ cycloalkyl, or C₂-C₈ heterocyclyl. In some embodiments, R_{A}¹² and R_{A}¹⁵ are each independently selected from COR_{A}²⁰, or CONR_{A}²⁰R_{A}²¹, wherein R_{A}²⁰ and R_{A}²¹ are each independently selected from C₁-C₈ alkyl. In some embodiments, R_{A}¹⁶ and R_{A}¹⁷ are each independently selected from hydrogen, C₁-C₈ alkyl, C₃-C₈ cycloalkyl, or C₂-C₈ heterocyclyl. In some embodiments, R_{A}¹⁶ and R_{A}¹⁷ together with the atom(s) to which they are connected form a 3-6 membered cycloalkyl or 3-6 membered heterocyclyl ring. In some embodiments, R_{A}¹⁸ and R_{A}²² are each independently selected from hydrogen, C₁-C₈ alkyl, C₃-C₈ cycloalkyl, or C₂-C₈ heterocyclyl. In some embodiments, R_{A}¹⁸ and R_{A}²² are each independently selected from H, CH₃, CH₂CH₃, CH(CH₃)₂, CF₃, CHF₂, cyclopropyl, or cyclobutyl.

In some embodiments, L³ is a bond, C₁-C₃ alkylene, C₃-C₈ cycloalkylene, C₂-C₈ heteroalkylene, C₂-C₈ heterocyclyl, —(C₁-C₃ alkylene)-(C₃-C₈ cycloalkylene)-, —(C₁-C₃ alkylene)-(C₂-C₈ heterocyclylene)-, or —(C₁-C₃ alkylene)-(C₂-C₈ heteroalkylene)-.

In some embodiments, L³ is a bond,

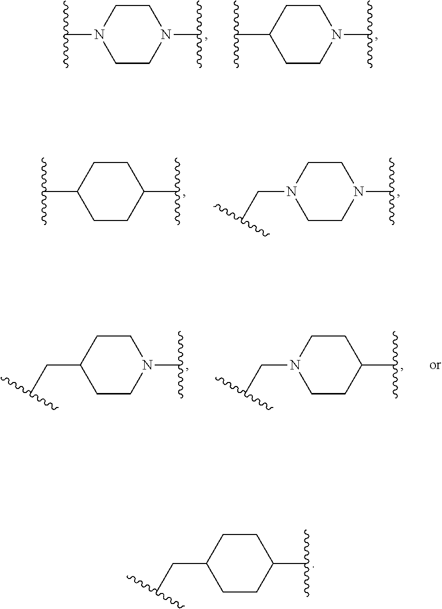

In some embodiments, the target protein binding moiety of Formula (A) is selected from:

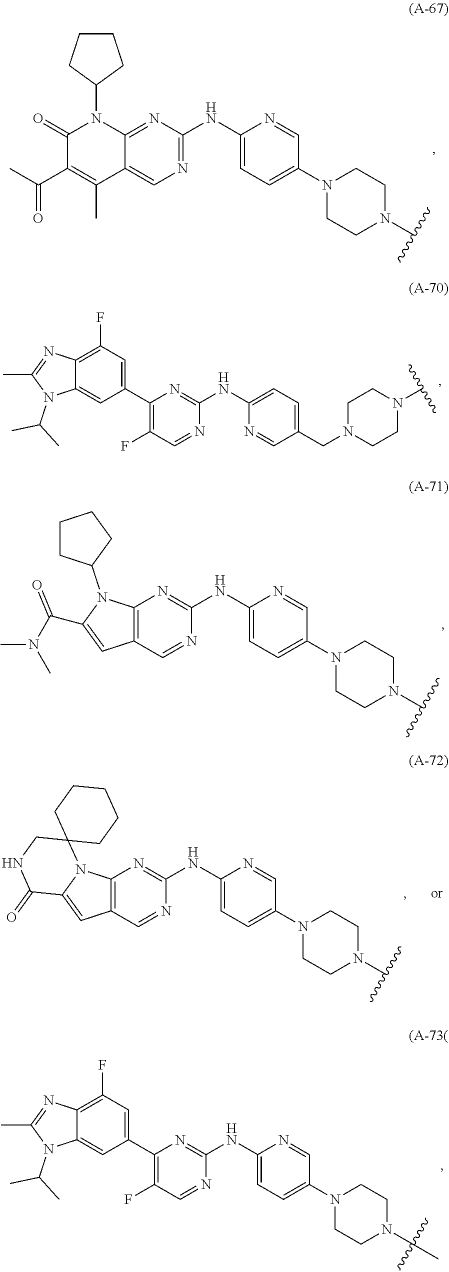 [SMILES: *N1CCN(c2ccc(Nc3ncc4c(C)c(C(C)=O)c(=O)n(C5CCCC5)c4n3)nc2)CC1 ; *N1CCN(Cc2ccc(Nc3ncc(F)c(-c4cc(F)c5nc(C)n(C(C)C)c5c4)n3)nc2)CC1 ; *N1CCN(c2ccc(Nc3ncc4cc(C(=O)N(C)C)n(C5CCCC5)c4n3)nc2)CC1 ; *N1CCN(c2ccc(Nc3ncc4cc5n(c4n3)C3(CCCCC3)CNC5=O)nc2)CC1 ; *N1CCN(c2ccc(Nc3ncc(F)c(-c4cc(F)c5nc(C)n(C(C)C)c5c4)n3)nc2)CC1]

or a pharmaceutically acceptable salt or solvate thereof.

In some embodiments, A is a target protein binding moiety comprising a CBP and/or p300 binding moiety.

In some embodiments, the target protein binding moiety has the structure of Formula (B-1), or a pharmaceutically acceptable salt or solvate thereof:

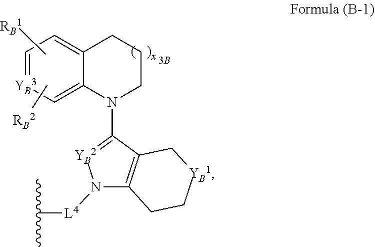

wherein,
Y_{B}¹ is CHR_{B}⁴ or NR_{B}⁴;
Y_{B}² is CH or N;
Y_{B}³ is CR_{B}² or N;
R_{B}¹ is a an optionally substituted 5-6 membered heteroaryl;
each R_{B}² is independently hydrogen, halogen, CN, NO₂, C₁-C₈ alkyl, C₁-C₈ haloalkyl, C₁-C₈ alkoxy, C₁-C₈ heteroalkyl, C₃-C₈ cycloalkyl, or C₂-C₈ heterocyclyl;
R_{B}⁴ is —C(═O)R_{B}⁸, —C(═O)OR_{B}⁸, —C(═O)NR_{B}⁶R_{B}⁷, or —NR_{B}⁶C(═O)R_{B}⁸;
L⁴ is a divalent group selected from —R_{B}^{3A}—R_{B}^{3B}—, wherein
R_{B}^{3A} and R_{B}^{3B} are each independently a bond, —O—, —S—, —NR_{B}⁵—, —C(═O)—, —C(═O)NR_{B}⁵—, —S(═O)—, —S(═O)NR_{B}⁵—, —S(═O)₂—, —S(═O)₂NR_{B}⁵—, C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₃ cycloalkylene, C₂-C₁₂ heterocyclene, arylene, or heteroarylene;
R_{B}⁵, R_{B}⁶, R_{B}⁷ and R_{B}⁸ are each independently selected from C₁-C₈ alkyl, C₁-C₈ haloalkyl, C₁-C₈ alkoxyalkyl, C₁-C₈ heteroalkyl, C₃-C₈ cycloalkyl, C₂-C₈ heterocyclyl, aryl, or heteroaryl, or
R_{B}⁶ and R_{B}⁷ together with the atom(s) to which they are connected optionally form a 3-20 membered heterocyclyl ring; and
x_{3B} is 0, 1, or 2.

In some embodiments, the target protein binding moiety of Formula (B-1) has the structure of Formula (B-2), or a pharmaceutically acceptable salt or solvate thereof:

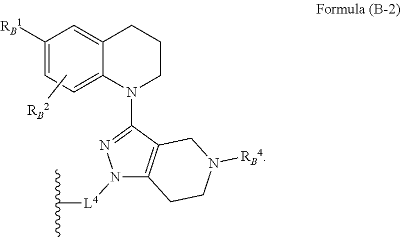

In some embodiments, R_{B}⁴ is —C(═O)R_{B}⁸, or —C(═O)NHR_{B}⁸, wherein R_{B}⁸ is C₈-C₅ alkyl. In some embodiments, R_{B}⁴ is —C(═O)R_{B}⁸, or —C(═O)NHR_{B}⁸, wherein R_{B}⁸ is CH₃. In some embodiments, R_{B}² is halogen, CN, NO₂, C₁-C₈ alkyl, C₁-C₈ haloalkyl, or C₁-C₈ alkoxy. In some embodiments, R_{B}² is CHCF₂. In some embodiments, R_{B}¹ is an optionally substituted 5-membered heteroaryl selected from pyrrolyl, furanyl, imidazolyl, pyrazolyl, oxazolyl, isoxazolyl, thienyl, thiazolyl, isothiazolyl, triazolyl, oxadiazolyl, thiadiazolyl, or tetrazolyl. In some embodiments, R_{B}¹ is an optionally substituted pyrazolyl. In some embodiments, R_{B}¹ is a methyl substituted pyrazolyl. In some embodiments, L⁴ is a bond, C₁-C₃ alkylene, C₃-C₈ cycloalkylene, C₂-C₈ heteroalkylene, C₂-C₈ heterocyclene, —(C₁-C₃ alkylene)-(C₃-C₈ cycloalkylene)-, —(C₁-C₃ alkylene)-(C₂-C₈ heterocyclene)-, or —(C₁-C₃ alkylene)-(C₂-C₈ heteroalkylene)-.

In some embodiments, the target protein binding moiety of Formula (B-1) is:

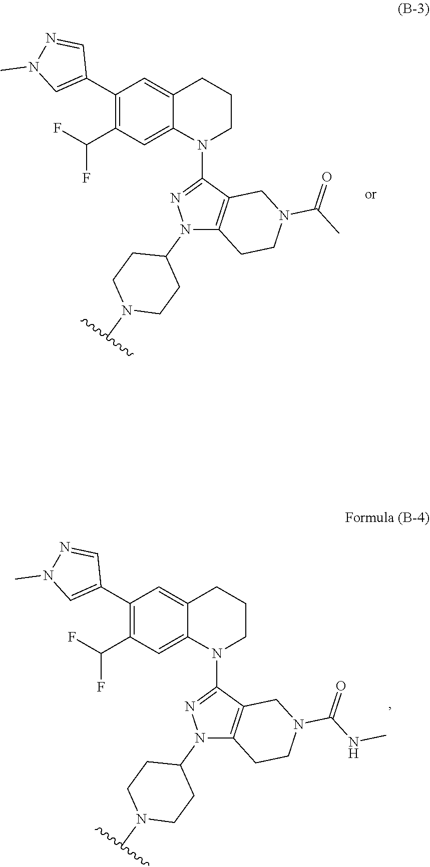

or a pharmaceutically acceptable salt or solvate thereof.

In some embodiments, A is a target protein binding moiety comprising a BET bromodomain-containing protein binding moiety.

In some embodiments, the target protein binding moiety has the structure of Formula (C-1), (C-2), (C-3), (C-4), (C-5), or (C-6), or a pharmaceutically acceptable salt or solvate thereof:

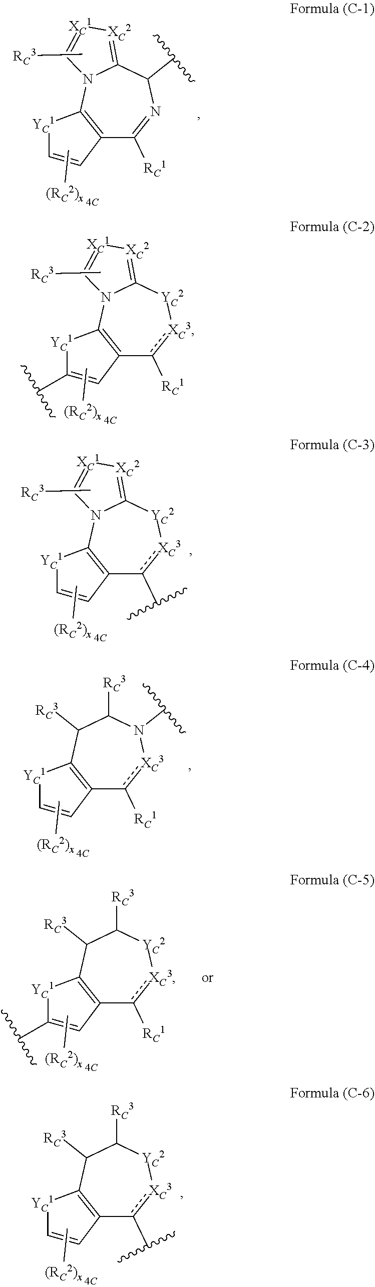

wherein,

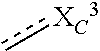

is

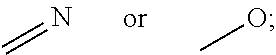

X_{C}¹ and X_{C}² are each independently CR_{C}³ or N;
Y_{C}¹ is O, S, or —C(R_{C}²)═C(R_{C}²)—;
Y_{C}² is C(R_{C}⁷)₂, or NR_{C}⁷;
R_{C}¹ is hydrogen or optionally substituted C₆-C₁₀ aryl or 5 to 10 membered heteroaryl;
each R_{C}² is independently hydrogen, halogen, CN, NO₂, NR_{C}⁴R_{C}⁵, —C(═O)R_{C}⁶, —C(═O)OR_{C}⁴, —C(═O)NR_{C}⁴R_{C}⁵, —OC(═O)R_{C}⁶, —N(R_{C}⁴)C(═O)R_{C}⁶, C₁-C₈ alkyl, C₁-C₈ heteroalkyl, C₂-C₈ alkynyl, C₁-C₈ haloalkyl, C₁-C₈ alkoxy, C₁-C₈ alkoxyalkyl, or C₁-C₈ alkylaryl;
each R_{C}³ is independently hydrogen, halogen, CN, NO₂, NR_{C}⁴R_{C}⁵, C₁-C₈ alkyl, C₁-C₈ haloalkyl, C₁-C₈ alkoxy, C₁-C₈ alkoxyalkyl, aryl, or heteroaryl;
R_{C}⁴, R_{C}⁵ and R_{C}⁶ are each independently selected from hydrogen, C₁-C₈ alkyl, C₁-C₈ haloalkyl, C₁-C₈ alkoxyalkyl, C₁-C₈ heteroalkyl, C₃-C₈ cycloalkyl, C₂-C₈ heterocyclyl, aryl, or heteroaryl, or
R_{C}⁴ and R_{C}⁵ together with the atom(s) to which they are connected optionally form a 3-20 membered heterocyclyl ring;
each R_{C}⁷ is independently hydrogen, NR_{C}⁴R_{C}⁵, OR_{C}⁴, —C(═O)R_{C}⁶, —C(═O)OR_{C}⁶, —C(═O)NR_{C}⁴R_{C}⁵, —(C₁-C₈ alkyl)-C(═O)NR_{C}⁴R_{C}⁵, —OC(═O)R_{C}⁶, —N(R_{C}⁸)C(═O)R_{C}⁶, C₁-C₈ alkyl, C₁-C₈ haloalkyl, C₁-C₈ heteroalkyl, C₃-C₈ cycloalkyl, C₂-C₈ heterocyclyl, or
two of R_{C}⁷, together with the atom(s) they are connected, optionally form a C₃-C₈ cycloalkyl, or C₂-C₈ heterocyclyl; and
x_{4C} is 1, 2, or 3.

In some embodiments,

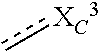

is

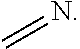

In some embodiments,

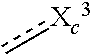

is

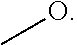

In some embodiments, X_{C}¹ and X_{C}² are each independently N. In some embodiments, Y_{C}¹ is S. In some embodiments, Y_{C}¹ is —C(R_{C}²)═C(R_{C}²)—. In some embodiments, Y_{C}² is C(R_{C}⁷)₂, In some embodiments, Y_{C}² is NR_{C}⁷. In some embodiments, R_{C}³ is hydrogen, halogen, C₁-C₈ alkyl, C₁-C₈ haloalkyl, C₁-C₈ alkoxy, or C₁-C₈ alkoxyalkyl. In some embodiments, each R_{C}² is independently hydrogen, halogen, C₁-C₈ alkyl, C₂-C₈ alkynyl, C₁-C₈ haloalkyl, C₁-C₈ alkoxy, C₁-C₈ alkoxyalkyl, aryl, or heteroaryl. In some embodiments, R_{C}¹ is optionally substituted C₆-C₁₀ aryl, optionally substituted with 1-4 halogen, CN, NO₂, NR_{C}⁴R_{C}⁵, —C(═O)R_{C}⁶, —C(═O)OR_{C}⁶, —C(═O)NR_{C}⁴R_{C}⁵, C₁-C₈ alkyl, C₁-C₈ haloalkyl, C₁-C₈ alkoxy, or C₁-C₈ alkoxyalkyl. In some embodiments, x₄c is 2; and each R_{C}² is independently C₁-C₈ alkyl. In some embodiments, x_{4C} is 2; and each R_{C}² is independently C₁-C₈ alkoxy.

In some embodiments, the target protein binding moiety is:

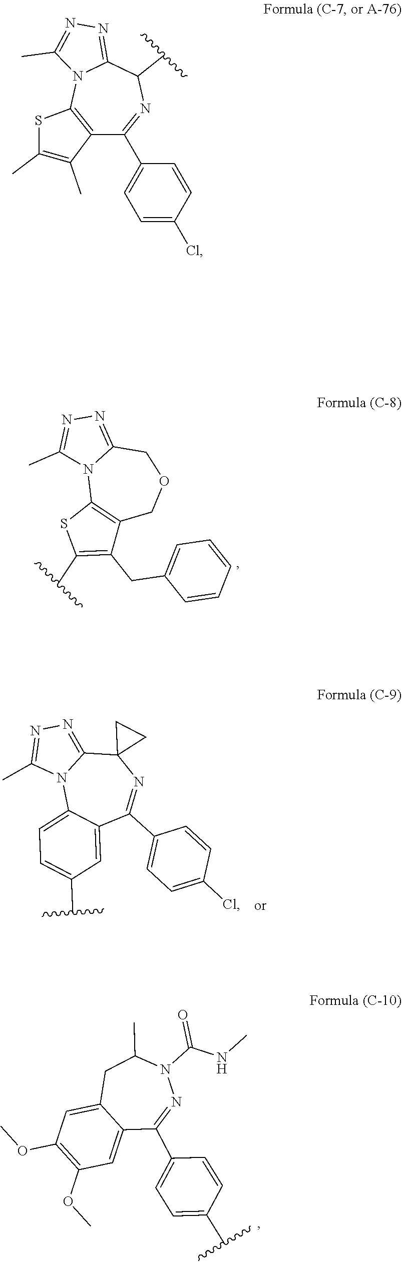

or a pharmaceutically acceptable salt or solvate thereof.

In some embodiments, the DDB1 binding moiety binds to a binding region on the DDB1 protein. In some embodiments, the DDB1 binding moiety binds non-covalently to the binding region. In some embodiments, the binding region comprises a beta propeller domain. In some embodiments, the beta propeller domain comprises a beta propeller C (BPC) domain. In some embodiments, the binding region comprises a top face of the BPC domain.

In some embodiments, the binding region comprises one or more of the following DDB1 residues: ARG327, LEU328, PRO358, ILE359, VAL360, ASP361, GLY380, ALA381, PHE382, SER720, ARG722, LYS723, SER738, ILE740, GLU787, TYR812, LEU814, SER815, ALA834, VAL836, ALA841, ALA869, TYR871, SER872, MET910, LEU912, TYR913, LEU926, TRP953, SER955, ALA956, ASN970, ALA971, PHE972, PHE1003, ASN1005, VAL1006, or VAL1033.

In some embodiments, the binding between the DDB1 binding moiety and the binding region comprises a binding affinity with an equilibrium dissociation constant (Kd) below 100 μM, a Kd below 90 μM, a Kd below 80 μM, a Kd below 70 μM, a Kd below 60 μM, a Kd below 50 μM, a Kd below 45 μM, a Kd below 40 μM, a Kd below 35 μM, a Kd below 30 μM, a Kd below 25 μM, a Kd below 20 μM, a Kd below 15 μM, a Kd below 14 μM, a Kd below 13 μM, a Kd below 12 μM, a Kd below 11 μM, a Kd below 10 μM, a Kd below 9 μM, a Kd below 8 μM, a Kd below 7 μM, a Kd below 6 μM, a Kd below 5 μM, a Kd below 4 μM, a Kd below 3 μM, a Kd below 2 μM, or a Kd below 1 μM. In some embodiments, the binding between the DDB1 binding moiety and the binding region comprises a binding affinity with a Kd<20 μM, a Kd from 20-100 μM, or a Kd>100 μM.

In another aspect, provided herein is an in vivo modified protein comprising a DNA damage-binding protein 1 (DDB1) protein directly bound to a DDB1 ligand, wherein the DDB1 ligand comprises the heterobifunctional compound of described herein.

In another aspect, provided herein is a method of degrading a target protein, comprising contacting the target protein with the heterobifunctional compound described herein.

In some embodiments, contacting the target protein with the heterobifunctional compound comprises contacting a cell comprising the target protein with the heterobifunctional compound described herein. In some embodiments, contacting the target protein with the heterobifunctional compound comprises administering the heterobifunctional compound to a subject comprising the cell. In some embodiments, the contact results in degradation of the target protein. In some embodiments, degradation is determined by an immunoassay. In some embodiments, degradation is ubiquitin-mediated. In some embodiments, degradation is by a proteasome.

Described herein are modified proteins and protein-ligand complexes. The modified proteins and protein-ligand complexes of some embodiments are useful for biotechnology applications such as selective degradation of a target protein, molecular glues, or anti-microbial drugs.

### INCORPORATION BY REFERENCE

All publications, patents, and patent applications mentioned in this specification are herein incorporated by reference for the specific purposes identified herein.

### BRIEF DESCRIPTION OF THE DRAWINGS

FIG. 1 show SPR sensorgrams of heterobifunctional compounds CPD-004 (A) and CPD-031 (B) binding to DDB1.

FIG. 2 shows immunoblots of cyclin D1, cyclin D2, cyclin D3, CDK4, CDK6, cleaved caspase-3 and p-Rb proteins expressed by Calu-1 cells (A) or of cyclin D1, cyclin D3, CDK4 and CDK6 proteins expressed by BT-549 cells (B) after treatment with a dose range of CDK4/6 inhibitor palbociclib or heterobifunctional compounds CPD-002, or CPD-004 for 16 hours.

FIG. 3 show immunoblots of cyclin D1, cyclin D2, cyclin D3, CDK4, CDK6 and p-Rb proteins expressed by Calu-1 cells after treatment with a dose range of heterobifunctional compounds CPD-031 for 16 hours.

FIG. 4 show immunoblots of cyclin D1, cyclin D2, cyclin D3, CDK4, CDK6 and p-Rb proteins expressed by Calu-1 cells after treatment with heterobifunctional compounds CPD-002 (A), or CPD-031 (B) at various time points.

FIG. 5 show immunoblots of cyclin D1, cyclin D2 and cyclin D3 proteins expressed by Calu-1 cells after treatment with heterobifunctional compounds CPD-002 and CPD-004 (A), or CPD-031 (B) in the presence or absence of MLN4924 (MLN), MG-132 (MG), or TAK-243 (TAK), and immunoblots of cyclin D1 proteins expressed in parental or DDB1 knockout Hs578T cells after treatment with heterobifunctional compound CPD-031 at indicated concentrations for 4 hours (C).

FIG. 6 show immunoblots of cyclin D1, cyclin D2, cyclin D3, and CDK4 proteins expressed by Calu-1 cells after treatment with a dose range of control compounds CPD-042 (A), or CPD-049 (B) for 16 hours, and anti-viability curves of Calu-1 cells in the presence of CPD-002 and CPD-042 (C), or CPD-031 and CPD-049 (D).

FIG. 7 shows anti-viability curves of Calu-1, NCI-H522, BT-549, Hs578T, or MIA PaCa-2 cells in the presence of palbociclib, ribociclib, abemaciclib, CPD-002, or CPD-031.

FIG. 8 shows immunoblots of P300 and CBP proteins expressed by LNCaP, Calu-1, NCI-H1703, or MM.1R cells after treatment with a dose range of heterobifunctional compound CPD-191 for 8 hours.

FIG. 9 shows immunoblots of BRD4 proteins expressed by Daudi, SU-DHL-4, or MDA-MB-231 cells after treatment with a dose range of heterobifunctional compound CPD-253 for 8 hours.

FIG. 10A-10B show immunoblots of cyclin D1, cyclin D3, CDK4, p-Rb, FoxM1 and cyclin A2 proteins expressed by T47D cells after treatment with a dose range of heterobifunctional compound CPD-343, or its control compound CPD-380 for 48 hours (FIG. 10A), and anti-viability curves of T47D cells in the presence of CP-343, or CPD-380 for 6 days (FIG. 10B).

FIG. 11A-11B show immunoblots of cyclin D1, CDK4, and CDK6 proteins expressed by Calu-1 cells after treatment with a dose range of heterobifunctional reference compound CP-10, or BSJ-03-123 for 8 hours (FIG. 11A), and anti-viability curves of Calu-1 cells in the presence of CP-10, or BSJ-03-123 for 3 days (FIG. 11B).

FIG. 12 shows flow cytometric analysis of Annexin V/7-AAD stained T47D cells after treatment with DMSO, palbociclib, heterobifunctional compound CPD-343, or control compound CPD-380 at indicated concentrations for 6 days.

FIG. 13 shows anti-viability curves of T47D parental or palbociclib-resistant cells in the presence of palbociclib, or heterobifunctional compound CPD-343 for 6 days.

### DETAILED DESCRIPTION OF THE INVENTION

DDB1 (damaged DNA binding protein 1) was first identified as a subunit of the heterodimeric complex involved in DNA repair. Later, it was discovered that DDB1 functions as a linker protein to connect substrate receptor proteins to CUL4 to assemble multiple CUL4-RING E3 ligase complexes (CRL4). The CRL family of E3 ligases is frequently hijacked by various viruses to degrade different host restriction factors, likely due to the intrinsic flexibility of the CRL ligases. Notably, DDB1 is among the most frequently hijacked E3 factors. Structural analysis of DDB1 in complex with HBx or SV5-V H-Box motifs have provided critical insights of the binding site of DDB1.

Disclosed herein are heterobifunctional compounds that modulate the protein level of either cyclin D, P300/CBP, or BRD4. These inhibitors were developed through recruiting DDB1 E3 ubiquitin ligase in an approach that permits more flexible regulation of protein levels in vitro and in vivo when compared with techniques such as gene knockout or short hairpin RNA-mediated (shRNA) knockdown. Unlike gene knockout or shRNA knockdown, a small molecule approach further provides an opportunity to study dose and time dependency in a disease model through modulating the administration routes, concentrations, and frequencies of administration of the corresponding heterobifunctional small molecule compound. These compounds were designed by incorporating three moieties: DDB1 ligands, linkers and CDK4/6, P300/CBP, or BRD4 binders.

Compounds described herein may be useful for several purposes, including but not limited to use as: 1) antiviral drugs; 2) DDB1 protein level modulators (e.g., increasing or decreasing DDB1 protein levels); 3) DDB1 function modulators (e.g., DDB1 activators or inhibitors); 4) molecular glues (e.g., increasing a protein-protein interaction between DDB1 and a second protein); or 5) targeted protein degraders. The molecular glue or targeted protein degradation functions may be useful for affecting activity or protein levels of a second protein.

### Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, R^{a}, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Haloalkyl” refers to an alkyl group that is substituted by one or more halogens. Exemplary haloalkyl groups include trifluoromethyl, difluoromethyl, trichloromethyl, 2,2,2 trifluoroethyl, 1,2 difluoroethyl, 3 bromo 2 fluoropropyl, and 1,2 dibromoethyl.

“Heteroalkyl”, “heteroalkenyl” and “heteroalkynyl” refer to substituted or unsubstituted alkyl, alkenyl and alkynyl groups which respectively have one or more skeletal chain atoms selected from an atom other than carbon. Exemplary skeletal chain atoms selected from an atom other than carbon include, e.g., O, N, P, Si, S, or combinations thereof, wherein the nitrogen, phosphorus, and sulfur atoms may optionally be oxidized and the nitrogen heteroatom may optionally be quaternized. If given, a numerical range refers to the chain length in total. For example, a 1- to 8-membered heteroalkyl has a chain length of 1 to 8 atoms, including both carbon and heteroatoms. Such a heteroalkyl chain may be referred to herein as a “C₁-C₈ heteroalkyl”. The same heteroalkyl chain may be referred to in the alternative as a 1-8 membered heteroalkyl. Connection to the rest of the molecule may be through either a heteroatom or a carbon in the heteroalkyl, heteroalkenyl or heteroalkynyl chain. Unless stated otherwise specifically in the specification, a heteroalkyl, heteroalkenyl, or heteroalkynyl group is optionally substituted by one or more substituents such as those substituents described herein. Bivalent heteroalkyl, heteroalkenyl and heteroalkynyl moieties may be referred to respectively as heteroalkylene, heteroalkenylene or heteroalkynylene moieties. It will be understood that the number and location of heteroatoms in a saturated or unsaturated heteroalkyl chain is limited to extent that such compounds are chemically stable (i.e., excluding peroxide moieties and the like).

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Bivalent alkenyl moieties may be referred to as alkenylene moieties. Unless stated otherwise specifically in the specification, an alkenyl group is optionally substituted by one or more of the following substituents: halo, cyano, nitro, oxo, thioxo, imino, oximo, trimethylsilanyl, R^{a}, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Bivalent alkynyl moieties may be referred to as alkynylene moieties. Unless stated otherwise specifically in the specification, an alkynyl group is optionally substituted by one or more of the following substituents: halo, cyano, nitro, oxo, thioxo, imino, oximo, trimethylsilanyl, R^{a}, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₈ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, R^{a}, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. Bivalent aryl moieties may be referred to as arylene moieties. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{a}, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms (i.e., a “C₃-C₁₅ cycloalkyl”). Such a cycloalkyl ring systems may be referred to in the alternative as a 3-15 membered cycloalkyl. In certain embodiments, a carbocyclyl comprises three to ten carbon atoms (i.e., a “C₃-C₁₀ cycloalkyl”). In other embodiments, a carbocyclyl comprises three to eight carbon atoms (i.e., a “C₃-C₈ cycloalkyl”) or five to seven carbon atoms (i.e., a “C₅-C₇ cycloalkyl”). The carbocyclyl may be attached to the rest of the molecule by a single bond or an exocyclic double bond. A carbocyclyl may be fully saturated (i.e., containing single C—C bonds only) or partially unsaturated (i.e., containing one or more double bonds or triple bonds). A fully saturated carbocyclyl radical is also referred to as “cycloalkyl.” Partially unsaturated carbocyclyl rings may be referred to as cyclo-alkenyl or cycloalkynyl moieties. Bivalent cycloalkyl moieties may be referred to as cycloalkylene moieties.

Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{a}, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical are optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” or “heterocycloalkyl” refers to a stable 3- to 20-membered non-aromatic ring radical that comprises two to fourteen carbon atoms and from one to six heteroatoms selected from nitrogen, oxygen and sulfur (i.e., N, O and S(O)_{z}, where z is 0, 1 or 2). Such a ring system may be referred to herein as a “C₂-C₄ heterocyclyl” or in the alternative as a 3-20 membered heterocyclyl. Similarly, a “C₂-C₅ heterocyclyl” refers to a ring system containing 2-8 carbon atoms and 1-6 heteroatoms, and preferably 1-3 heteroatoms, which ring system may be referred to in the alternative as a 3-14 membered heterocyclyl. In some embodiments herein, the heterocyclyl ring system comprises a 5-6 membered heterocyclyl, a 3-8 membered heterocyclyl, a 3-10 membered heterocyclyl, or a 3-13 membered heterocyclyl, wherein each such heterocyclyl preferably contains from 1-3 heteroatoms. Bivalent heterocycloalkyl moieties may be referred to as heterocyclene moieties. Unless stated otherwise specifically in the specification, the heterocyclyl radical is a monocyclic, bicyclic, tricyclic or tetracyclic ring system, which optionally includes fused or bridged ring systems. It will be understood that the number and location of heteroatoms in a heterocyclic ring is limited to extent that such compounds are chemically stable. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{a}, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heteroaryl” refers to a radical derived from a 3- to 18-membered aromatic ring radical that comprises two to seventeen carbon atoms and from one to six heteroatoms selected from nitrogen, oxygen and sulfur. Bivalent heteroaryl moieties may be referred to as heteroarylene moieties. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s).

Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]-thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl).

Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{a}, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para- isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

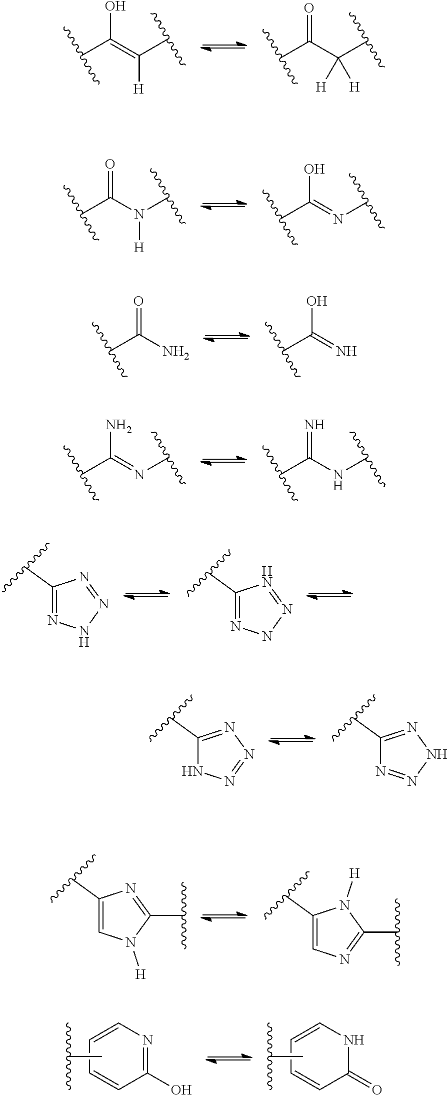

The compounds disclosed herein, in some embodiments, are used in different enriched isotopic forms, e.g., enriched in the content of ²H, ³H, ¹¹C, ¹³C and/or ¹⁴C. In one embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### Heterobifunctional Compounds

Provided herein, in some embodiments are heterobifunctional compounds and pharmaceutical compositions comprising said compounds. In some embodiments a heterobifunctional compound described herein comprises a DNA damage-binding protein 1 (DDB1) binding moiety, a linker, and/or a target protein binding moiety. In some embodiments a heterobifunctional compound described herein comprises a DDB1 binding moiety and a target protein binding moiety. In some embodiments, the heterobifunctional compound comprising a DDB1 binding moiety covalently connected through a linker to a target protein binding moiety. In some embodiments, a DDB1 binding moiety is a natural product. In some embodiments, a DDB1 binding moiety is a synthetic product. In some embodiments, a target protein binding moiety is configured to bind a target protein.

In one aspect, provided herein is a heterobifunctional compound of Formula (I), or a pharmaceutically acceptable salt or solvate thereof:

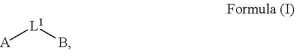

wherein, A is a target protein binding moiety; L¹ is a linker; and B is a DDB1 binding moiety.

In another aspect, described herein is a compound comprising a DNA damage-binding protein 1 (DDB1) binding moiety. In some embodiments, the compound comprises a DBB1 binding moiety, but does not comprise a linker and/or a target protein binding moiety. Representative examples of such DDB1 binding compounds are shown in Table 1. In some embodiments, the compound comprises a DBB1 binding moiety and linker, but does not comprise a target protein. Representative examples of such compounds are shown in Table 2.

### DDB1 Binding Moieties

Disclosed herein, in some embodiments, are compounds comprising a DDB1 binding moiety. The compound may consist of a DDB1 binding moiety or may be comprise a heterobifunctional molecule comprising the DDB1 binding moiety. In some embodiments, the compounds comprising only a DDB1 moiety. The compound may be useful for any of the aspects disclosed herein.

In preferred embodiments, the DDB1 binding moiety has the structure of Formula (II), or a pharmaceutically acceptable salt or solvate thereof:

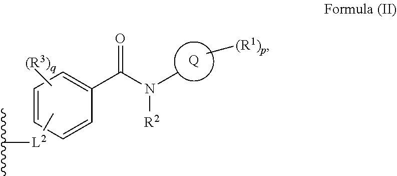

wherein,
ring Q is phenyl or a 5 or 6-membered monocyclic heteroaryl;
L² is a bond, —O—, —NR^{4A}—, —NR^{4B}—C(═O)—, —NR^{4B}—C(═O)—(C₁-C₃alkylene)-NR^{4A}—, —NR^{4B}—C(═O)—(C₁-C₃alkylene)-O—, —(C₁-C₃alkylene)-NR^{4B}—C(═O)—, —C(═O)NR^{4A}—, —C₁-C₃alkylene-, —C₂-C₃ alkenylene-, —C₂-C₃alkynylene-, C₃-C₈ cycloalkylene, or C₂-C₈ heterocyclene;
each R¹ is independently hydrogen, halogen, —CN, NO₂, —OR^{4A}, —NR^{4A}R^{4B}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl or heteroaryl, or
two R¹, together with the atom(s) to which they are connected, optionally form C₃-C₁₃ cycloalkyl, C₂-C₁₂ heterocyclyl, aryl, or heteroaryl;
R² is hydrogen, C₁-C₆ alkyl, C₃-C₈ cycloalkyl, OH, or O—C₁-C₄ alkyl;
each R³ is independently hydrogen, halogen, —CN, —NO₂, —OR^{4A}, —NR^{4A}R^{4B}, —C(═O)R^{4A}, —C(═O)OR^{4A}, —C(═O)NR^{4B}R^{4A}, —OC(═O)R^{4A}, —N(R^{4A})C(═O)R^{4B}, C₁-C₆ alkyl, C₁-C₆ haloalkyl, C₁-C₆ heteroalkyl, C₃-C₈ cycloalkyl, C₂-C₈ heterocyclyl, aryl, or heteroaryl, or
two R³, together with the atom(s) to which they are connected, optionally form C₃-C₁₃ cycloalkyl, C₂-C₁₂ heterocyclyl, aryl, or heteroaryl;
each R^{4A} and R^{4B} is independently hydrogen, C₁-C₆ alkyl, C₂-C₆ alkenyl, C₂-C₆ alkynyl, C₁-C₆ haloalkyl, C₁-C₆ heteroalkyl, C₃-C₈ cycloalkyl, C₂-C₈ heterocyclyl, aryl, or heteroaryl, or
R^{4A} and R^{4B}, together with the atom(s) to which they are connected, optionally form C₂-C₁₂ heterocyclyl;
p is 1, 2 or 3; and
q is 1, 2 or 3.

In some embodiments of Formula (II), L² is para to the carboxamido moiety. In some embodiments of Formula (II), L² is meta to the carboxamido moiety. In some embodiments of Formula (II), L² is ortho to the carboxamido moiety.

In some embodiments, the DDB1 binding moiety has the structure of Formula (II′), In some embodiments, the DDB1 binding moiety has the structure of Formula (II′), or a pharmaceutically acceptable salt or solvate thereof:

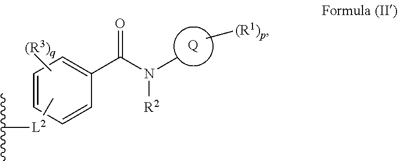

wherein,
ring Q is phenyl or a 5 or 6-membered monocyclic heteroaryl;
L² is absent, —O—, —NR^{4A}—, —NR^{4B}—C(═O)—, —NR^{4B}—C(═O)—(C₁-C₃alkylene)-NR^{4A}—, —NR^{4B}—C(═O)—(C₁-C₃alkylene)-O—, —(C₁-C₃alkylene)-NR^{4B}—C(═O)—, —C(═O)NR^{4A}—, —C₁-C₃alkylene-, —C₂-C₃ alkenylene-, —C₂-C₃alkynylene-, C₃-C₈ cycloalkyl, or C₂-C₈ heterocyclyl;
R¹ is hydrogen, halogen, —CN, —NO₂, —OR^{4A}, —NR^{4A}R^{4B}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl or heteroaryl, or
two R¹, together with the atom(s) to which they connected, optionally form C₃-C₁₃ cycloalkyl, C₂-C₁₂ heterocyclyl, aryl, or heteroaryl;
R² is hydrogen or C₁-C₆ alkyl, C₃-C₈ cycloalkyl, OH, or OR;
each R³ is independently hydrogen, halogen, —CN, —NO₂, —OR^{4A}, —NR^{4A}R^{4B}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl, or heteroaryl, or
two R³, together with the atom(s) to which they connected, optionally form C₃-C₁₃ cycloalkyl, C₂-C₁₂ heterocyclyl, aryl, or heteroaryl;
each R^{4A} and R^{4B} is independently hydrogen, C₁-C₆ alkyl, C₂-C₆ alkenyl, C₂-C₆ alkynyl, C₁-C₆ haloalkyl, C₁-C₆ heteroalkyl, C₃-C₈ cycloalkyl, C₂-C₈ heterocyclyl, aryl, or heteroaryl, or
R^{4A} and R^{4B}, together with the atom(s) to which they connected, optionally form C₂-C₁₂ heterocyclyl;
p is 1, 2 or 3; and
q is 1, 2 or 3.

In some embodiments, the DDB1 binding moiety has the structure of Formula (II″), or a pharmaceutically acceptable salt or solvate thereof:

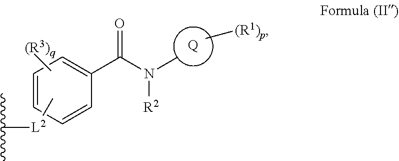

wherein,
ring Q is phenyl or a 5 or 6-membered monocyclic heteroaryl;
L² is absent, —O—, —NR^{4A}—, —NR^{4B}—C(═O)—, —NR^{4B}—C(═O)_(C₁-C₃alkylene)-NR^{4A}—, —NR^{4B}—C(═O)—(C₁-C₃alkylene)-O—, —(C₁-C₃alkylene)-NR^{4B}—C(═O)—, —C(═O)NR^{4A}—, —C₁-C₃alkylene-, —C₂-C₃ alkenylene-, —C₂-C₃alkynylene-, C₃-C₈ cycloalkyl, or 4 to 7-membered heterocyclyl;
R¹ is hydrogen, halogen, —CN, —OR^{4A}, —NR^{4A}R^{4B}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, 4 to 7-membered heterocyclyl, aryl or heteroaryl;
R² is hydrogen, C₁-C₆ alkyl, or C₃-C₈ cycloalkyl;
R³ is hydrogen, halogen, —CN, —OR^{4A}, —NR^{4A}R^{4B}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl, or heteroaryl;
each R^{4A} and R^{4B} is independently hydrogen, C₁-C₆ alkyl, C₂-C₆ alkenyl, C₂-C₆ alkynyl, C₁-C₆ haloalkyl, C₁-C₆ heteroalkyl, C₃-C₈ cycloalkyl, C₂-C₈ heterocyclyl, aryl, or heteroaryl;
p is 1, 2 or 3; and
q is 1, 2 or 3.

Each of the embodiments described herein for Formula (II) are also applicable to Formula (II′) or Formula (II″), to the extent the embodiments are not inconsistent with the definitions of Formula (II′) or Formula (II″). The description of Formula (II) may be replaced by the description of Formula (II′) or Formula (II″).

In some embodiments of the DDB1 binding moiety of Formula (II), ring Q is a 5-membered monocyclic heteroaryl. In some embodiments, ring Q is a 5-membered monocyclic heteroaryl comprising at least one N atom. In some embodiments, ring Q is selected from the group consisting of pyrrolyl, furanyl, imidazolyl, pyrazolyl, oxazolyl, isoxazolyl, thienyl, thiazolyl, isothiazolyl, triazolyl, oxadiazolyl, thiadiazolyl, or tetrazolyl. In some embodiments, ring Q is selected from the group consisting of furan, thienyl, oxazole, or thiazole. In some embodiments, ring Q is selected from the group consisting of imidazolyl or pyrazolyl. In some embodiments, ring Q is selected from the group consisting of pyrazolyl, or thiazolyl.

In some embodiments, the DDB1 binding moiety of Formula (II) has the structure of Formula (III-1), or a pharmaceutically acceptable salt or solvate thereof:

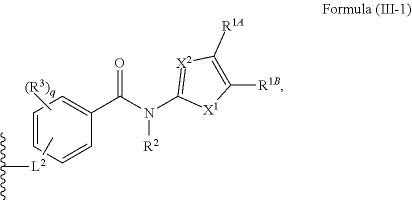

wherein,
X¹ is O, S, or NR⁵;
X² is N or CH;
R⁵ is hydrogen, C₁-C₆ alkyl, C₁-C₆ haloalkyl, C₁-C₆ heteroalkyl, C₃-C₈ cycloalkyl, C₂-C₈ heterocyclyl; and
R^{1A} and R^{1B} are independently selected from hydrogen, halogen, CN, —NO₂, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ heteroalkyl, C₁-C₆ haloalkyl, C₃-C₈ cycloalkyl, C₂-C₈ heterocyclyl, aryl or heteroaryl, or
R^{1A} and R^{1B}, together with the atom(s) to which they are connected, optionally form C₃-C₁₃ cycloalkyl, C₂-C₁₂ heterocyclyl, aryl, or heteroaryl.

In some embodiments, the DDB1 binding moiety of Formula (II) has the structure of Formula (III-2), or a pharmaceutically acceptable salt or solvate thereof:

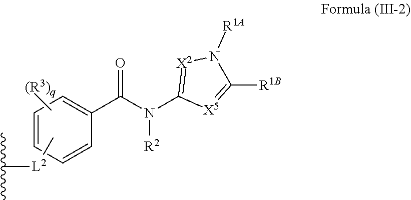

wherein,
X² and X⁵ are independently N or CH;
and
R^{1A} and R^{1B} are independently selected from hydrogen, halogen, CN, —NO₂, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ heteroalkyl, C₁-C₆ haloalkyl, C₃-C₈ cycloalkyl, C₂-C₈ heterocyclyl, aryl or heteroaryl, or
R^{1A} and R^{1B}, together with the atom(s) to which they are connected, optionally form C₃-C₁₃ cycloalkyl, C₂-C₁₂ heterocyclyl, aryl, or heteroaryl.

In some embodiments of Formulae (III-1) herein, X¹ is O or S; and X² is N. In some embodiments, X¹ is O or S; and X² is CH. In some embodiments, X¹ is O; and X² is N. In some embodiments, X¹ is S; and X² is N.

In some embodiments of Formulae (III-2) herein, X⁵ is CH. In some embodiments of Formulae (III-2) herein, X⁵ is CH; and X² is N. In some embodiments of Formulae (III-2) herein, X⁵ is CH; and X² is CH. In some embodiments, X⁵ is N. In some embodiments, X⁵ is N; and X² is N. In some embodiments, X⁵ is N; and X² is CH.

In some embodiments of Formula (II), (III-1) or (III-2) herein, R² is H. In some embodiments, R² is C₁-C₆ alkyl. In some embodiments, R² is methyl, ethyl, n-propyl, or isopropyl. In some embodiments, R² may include OH or O—C₁-C₄alkyl.

In some embodiments, the DDB1 binding moiety of Formula (II) has the structure of Formula (IV-1), or a pharmaceutically acceptable salt or solvate thereof:

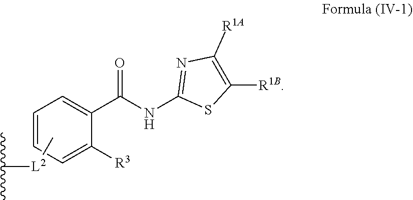

In some embodiments, the DDB1 binding moiety of Formula (II) has the structure of Formula (IV-2) or (IV-3), or a pharmaceutically acceptable salt or solvate thereof:

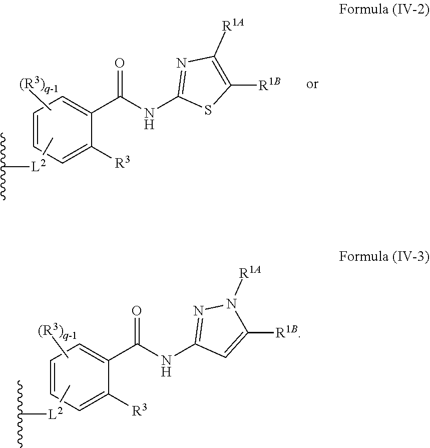

In some embodiments, the DDB1 binding moiety of Formula (II), has the structure of Formula (IVa), (IVb), (IVc) or (IVd), a pharmaceutically acceptable salt or solvate thereof:

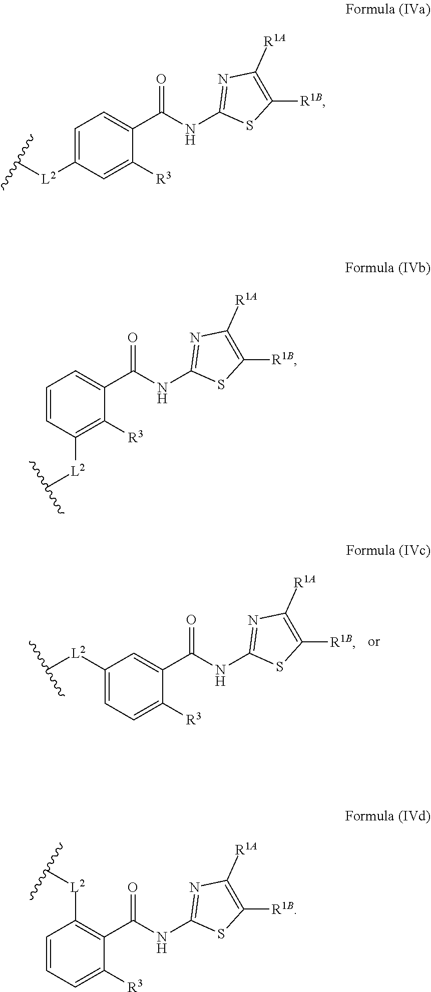

In some embodiments, the DDB1 binding moiety of Formula (II) has the structure of Formula (IV-4), or a pharmaceutically acceptable salt or solvate thereof:

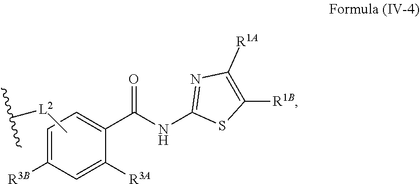

wherein,
R^{3A} and R^{3B} are each independently hydrogen, halogen, —CN, —NO₂, —OR^{4A}, —NR^{4A}R^{4B}, —C(═O)R^{4A}, —C(═O)OR^{4A}, —C(═O)NR^{4B}R^{4A}, —OC(═O)R^{4A}, —N(R^{4A})C(═O)R^{4B}, C₁-C₆ alkyl, C₁-C₆ haloalkyl, C₁-C₆ heteroalkyl, C₃-C₈ cycloalkyl, C₂-C₈ heterocyclyl, aryl, or heteroaryl; and
each R^{4A} and R^{4B} is independently hydrogen, C₁-C₆ alkyl, C₂-C₆ alkenyl, C₂-C₆ alkynyl, C₁-C₆ haloalkyl, C₁-C₆ heteroalkyl, C₃-C₈ cycloalkyl, C₂-C₈ heterocyclyl, aryl, or heteroaryl, or
R^{4A} and R^{4B}, together with the atom(s) to which they are connected, optionally form C₂-C₁₂ heterocyclyl;

In some embodiments, the DDB1 binding moiety of Formula (II), has the structure of Formula (IVe), (IVf), or (IVg), or a pharmaceutically acceptable salt or solvate thereof:

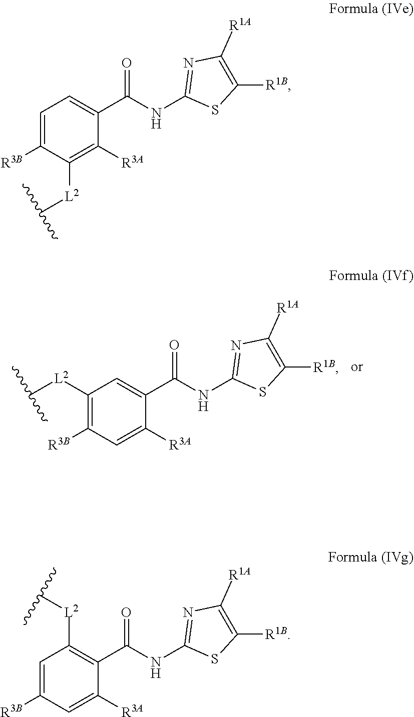

In some embodiments, the DDB1 binding moiety of Formula (II) has the structure of Formula (IV-5), or a pharmaceutically acceptable salt or solvate thereof:

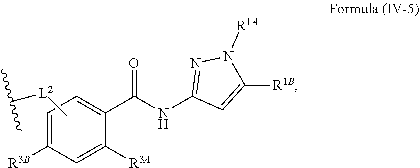

wherein,
R^{3A} and R^{3B} are each independently hydrogen, halogen, —CN, —NO₂, —OR^{4A}, —NR^{4A}R^{4B}, —C(═O)R^{4A}, —C(═O)OR^{4A}, —C(═O)NR^{4B}R^{4A}, —OC(═O)R^{4A}, —N(R^{4A})C(═O)R^{4B}, C₁-C₆ alkyl, C₁-C₆ haloalkyl, C₁-C₆ heteroalkyl, C₃-C₈ cycloalkyl, C₂-C₈ heterocyclyl, aryl, or heteroaryl; and
each R^{4A} and R^{4B} is independently hydrogen, C₁-C₆ alkyl, C₂-C₆ alkenyl, C₂-C₆ alkynyl, C₁-C₆ haloalkyl, C₁-C₆ heteroalkyl, C₃-C₈ cycloalkyl, C₂-C₈ heterocyclyl, aryl, or heteroaryl, or
R^{4A} and R^{4B}, together with the atom(s) to which they are connected, optionally form C₂-C₁₂ heterocyclyl.

In some embodiments, the DDB1 binding moiety of Formula (II), has the structure of Formula (IVh), (IVi), (IVj), or (IVk), or a pharmaceutically acceptable salt or solvate thereof:

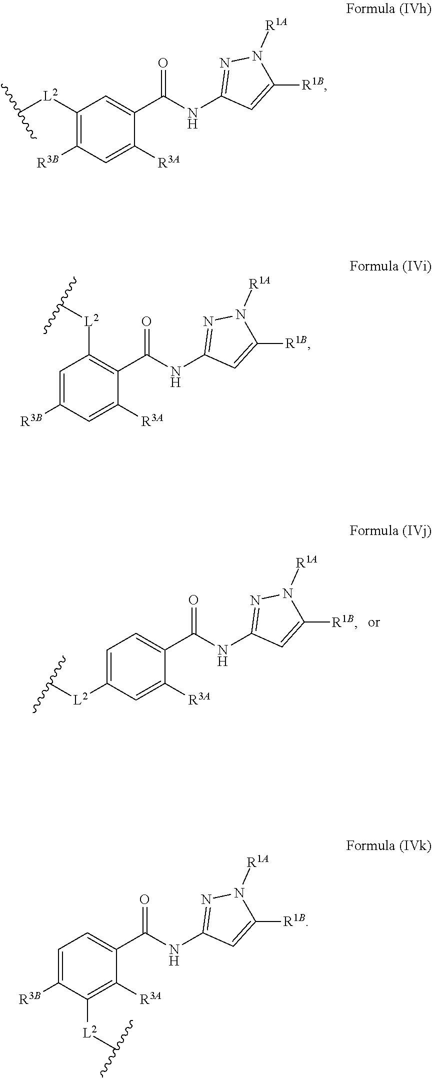

In some embodiments of Formulae (IV-1) to (IV-5) or (IVa) to (IVk), R^{1A} is selected from hydrogen, halogen, —OCH₃, —NH₂, —NHCH₃, —N(CH₃)₂, —C(═O)CH₃, —C(═O)OCH₃, —C(═O)NH₂, —C(═O)NHCH₃, —C(═O)N(CH₃)₂, —CH₃, —CHCF₂, —CF₃, —CH₂CH₃, —CH(CH₃)₂, —C(CH₃)₃, cyclopropyl, cyclobutyl, cyclopentyl, cyclohexyl, or phenyl. In some embodiments, R^{1A} is selected from hydrogen, halogen, —OCH₃, —C(═O)CH₃, —C(═O)OCH₃, —CH₃, —CF₃, —CH₂CH₃, —CH(CH₃)₂, —C(CH₃)₃, cyclopropyl, cyclobutyl, cyclopentyl, cyclohexyl, or phenyl. In some embodiments, R^{1A} is selected from hydrogen, —C(═O)CH₃, —C(═O)OCH₃, —CH₃, or phenyl.

In some embodiments, R^{1B} is selected from hydrogen, halogen, —OCH₃, —NH₂, —NHCH₃, —N(CH₃)₂, —C(═O)CH₃, —C(═O)OCH₃, —C(═O)NH₂, —C(═O)NHCH₃, —C(═O)N(CH₃)₂, —CHCF₂, —CF₃, or phenyl. In some embodiments, ^{1B} is selected from —CH₃, —CH(CH₃)₂, —C(CH₃)₃, cyclopropyl, cyclobutyl, cyclopentyl, or cyclohexyl. In some embodiments, R^{1B} is selected from hydrogen, halogen, —OCH₃, —C(═O)CH₃, —C(═O)OCH₃, —CF₃, or phenyl. In some embodiments, ^{1B} is selected from —CH₃, —CH(CH₃)₂, —C(CH₃)₃, cyclopropyl, cyclobutyl, cyclopentyl, or cyclohexyl.

In some embodiments, ring Q is a phenyl or 6-membered monocyclic heteroaryl. In some embodiments, ring Q is a phenyl. In some embodiments, ring Q is a 6-membered heteroaryl. In some embodiments, the 6-membered heteroaryl comprises at 1 to 2 N atoms. In some embodiments, ring Q is a 5-membered heteroaryl. In some embodiments, the 5-membered heteroaryl comprises at 1 to 2 N atoms. In some embodiments, ring Q is selected from pyridinyl, pyridazinyl, pyrazinyl, pyrimidinyl, or triazinyl. In some embodiments, ring Q is pyridinyl, pyrazinyl, or triazinyl. In some embodiments, ring Q is pyridinyl. In some embodiments, ring Q is pyrazinyl.

In some embodiments, the DDB1 binding moiety of Formula (II) has the structure of Formula (V-1), or a pharmaceutically acceptable salt or solvate thereof:

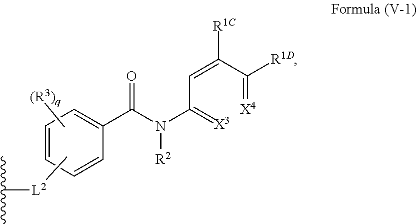

wherein,
X³ is N or CH;
X⁴ is N or CR^{1E}; and
each of R^{1C}, R^{1D}, and R^{1E} is independently selected from hydrogen, halogen, CN, —NO₂, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl or heteroaryl, or
R^{1C} and R^{1D}, or R^{1D} and R^{1E}, together with the atom(s) to which they are connected, optionally form C₃-C₁₃ cycloalkyl, C₂-C₁₂ heterocyclyl, aryl, or heteroaryl.

In some embodiments, the DDB1 binding moiety of Formula (II), has the structure of Formula (V-2), or a pharmaceutically acceptable salt or solvate thereof:

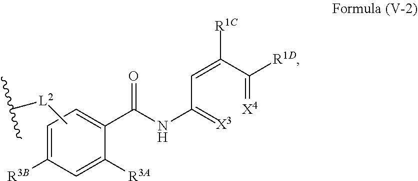

wherein,
X³, X⁴, R^{1C}, R^{1D}, and R^{1E} are defined as in Formula (V-1);
R^{3A} and R^{3B} are each independently hydrogen, halogen, —NO₂, —CN, —OR^{4A}, —NR^{4A}R^{4B}, —C(═O)R^{4A}, —C(═O)OR^{4A}, —C(═O)NR^{4B}R^{4A}, —OC(═O)R^{4A}, —N(R^{4A})C(═O)R^{4B}, C₁-C₆ alkyl, C₁-C₆ haloalkyl, C₁-C₆ heteroalkyl, C₃-C₈ cycloalkyl, C₂-C₈ heterocyclyl, aryl, or heteroaryl; and
each R^{4A} and R^{4B} is independently hydrogen, C₁-C₆ alkyl, C₂-C₆ alkenyl, C₂-C₆ alkynyl, C₁-C₆ haloalkyl, C₁-C₆ heteroalkyl, C₃-C₈ cycloalkyl, C₂-C₈ heterocyclyl, aryl, or heteroaryl, or
R^{4A} and R^{4B}, together with the atom(s) to which they are connected, optionally form C₂-C₁₂ heterocyclyl.

In some embodiments, X⁴ is N. In some embodiments, X⁴ is CR^{1E}.

In some embodiments, the DDB1 binding moiety of Formula (II) has the structure of Formula (V-3), or a pharmaceutically acceptable salt or solvate thereof:

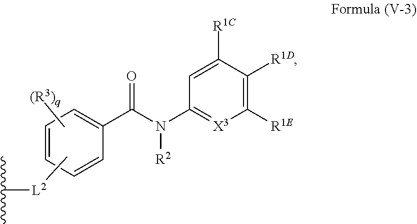

wherein,
X³, R^{1C}, R^{1D}, and R^{1E} are defined as in Formula (V-1).

In some embodiments, the DDB1 binding moiety of Formula (II) has the structure of Formula (VIa), (VIb), (VIc), or (VId), or a pharmaceutically acceptable salt or solvate thereof:

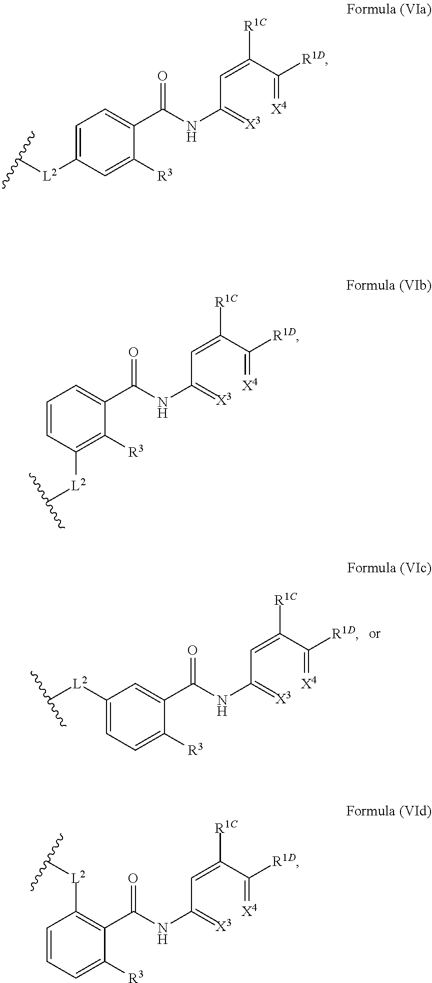

wherein,
X³, X⁴, R^{1C}, R^{1D}, and R^{1E} are defined as in Formula (V-1).

In some embodiments, the DDB1 binding moiety of Formula (II) has the structure of Formula (VIe), (VIf), or (VIg), or a pharmaceutically acceptable salt or solvate thereof:

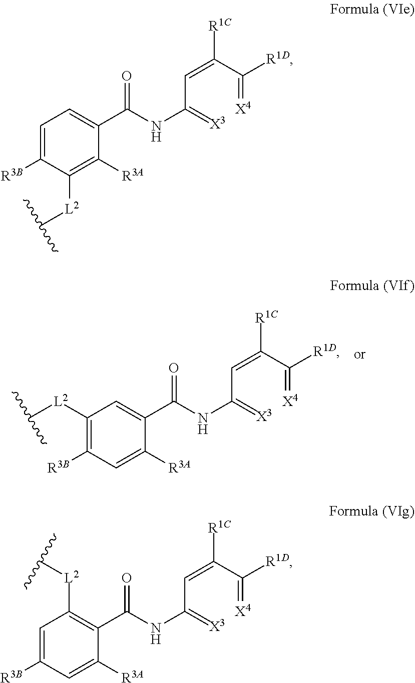

wherein,
X³, X⁴, R^{1C}, R^{1D}, and R^{1E} are defined as in Formula (V-1); and
R^{3A}, R^{3B}, R^{4A}, and R^{4B} are defined as in Formula (V-2).

In some embodiments of Formulae (V-1), (V-2), (V-3) or (VIa) to (VIg) herein, X³ is N. In other such embodiments, X³ is CH.

In some embodiments of Formulae (V-1), (V-2), (V-3) or (VIa) to (VIg) herein, R^{1C} and R^{1E} are each hydrogen; and R^{1D} is hydrogen, halogen, CN, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl, or heteroaryl. In some such embodiments, R^{1C} and R^{1E} are each hydrogen; and R^{1D} is halogen, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl, or heteroaryl.

In some embodiments, X³ and X⁴ are N; R^{1C} is hydrogen; and R^{1D} is hydrogen, halogen, —NO₂, CN, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl, or heteroaryl. In some embodiments, X³ and X⁴ are N; R^{1C} is hydrogen; and R^{1D} is hydrogen, halogen, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, 4 to 7-membered heterocycloalkyl, aryl, or heteroaryl. In some embodiments, X³ and X⁴ are N; R^{1C} is hydrogen; and R^{1D} is —OR^{4A}, —NR^{4B}R^{4A}, C₁-C₆ alkyl, C₁-C₆ haloalkyl, C₃-C₈ cycloalkyl, C₂-C₈ heterocyclyl. In some embodiments, X³ and X⁴ are N; R^{1C} is hydrogen; and R^{1D} is —NR^{4B}R^{4A}. In some embodiments, X³ and X⁴ are N; R^{1C} is hydrogen; and R^{1D} is —N(CH₃)₂.

In some embodiments, X³ is N; X⁴ is CR^{1E}; R^{1C} is hydrogen; and R^{1D} and R^{1E} are independently selected from hydrogen, halogen, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, 4 to 7-membered heterocycloalkyl, aryl, or heteroaryl. In some embodiments, X³ is N; X⁴ is CR^{1E}; R^{1C} is hydrogen; and R^{1D} and R^{1E} are independently selected from hydrogen, halogen, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, 4 to 7-membered heterocycloalkyl, aryl, or heteroaryl. In some embodiments, X³ is N; X⁴ is CR^{1E}; R^{1C} is hydrogen; and R^{1D} and R^{1E} are independently selected from hydrogen, halogen, —OR^{4A}, —NR^{4B}R^{4A}, C₁-C₆ alkyl, C₁-C₆ haloalkyl, C₁-C₆ heteroalkyl, C₃-C₈ cycloalkyl, or 4 to 7-membered heterocycloalkyl.

In some embodiments, X³ is N; X⁴ is CR^{1E}; R^{1C} is hydrogen; and R^{1D} and R^{1E}, together with the atom(s) to which they connected, form C₃-C₁₃ cycloalkyl, C₂-C₁₂ heterocyclyl, aryl, or heteroaryl.

In some embodiments, R^{1D} is C₁-C₆ alkyl, C₁-C₆ heteroalkyl, C₃-C₈ cycloalkyl, or C₂-C₈ heterocyclyl. In some embodiments, R^{1D} is methyl, difluoromethyl, trifluoromethyl, ethyl, n-propyl, isopropyl, cyclopropyl, or t-butyl. In some embodiments, R^{1D} is C₁-C₆ alkyl. In some embodiments, R^{1D} is methyl, ethyl, n-propyl, isopropyl, or t-butyl. In some embodiments, R^{1D} is methyl. In some embodiments, R^{1D} is hydrogen. In some embodiments, R^{1D} is —NR^{4B}R^{4A}. In some embodiments, R^{1D} is —NH₂, NH(CH₃), —N(CH₃)₂. In some embodiments, R^{1D} is —N(CH₃)₂. In some embodiments, R^{1D} is —OR^{4A}. In some embodiments, R^{1D} is —OH, —OCH₃, —OCHF₂, —OCF₃, —OCH(CH₃)₂, —O-cyclopropyl. In some embodiments, R^{1D} is —OCH₃. In some embodiments, R^{1D} is H.

In some embodiments, each R³ is independently hydrogen, halogen, C₁-C₆ alkyl, C₁-C₆ haloalkyl, C₁-C₆ heteroalkyl, C₁-C₆ alkoxy, C₁-C₆ alkylamino, C₁-C₆ cycloalkoxy, C₁-C₆ cycloalkylamino, C₃-C₈ cycloalkyl, or C₂-C₈ heterocyclyl. In some embodiments, R³ is F, Cl, Br, CH₃, CHF₂, CF₃, CH₂CH₃, CH(CH₃)₂, cyclopropyl, CN, —NH₂, NH(CH₃), NH(i-Pr), NH(n-Bu), NH(t-Bu), or N(CH₃)₂. In some embodiments, R³ is CH₃. In some embodiments, R³ is NH(CH₃).

In some embodiments, R^{3A} and R^{3B} are independently hydrogen, halogen, C₁-C₆ alkyl, C₁-C₆ haloalkyl, C₁-C₆ heteroalkyl, C₁-C₆ alkoxy, C₁-C₆ alkylamino, C₁-C₆ cycloalkoxy, C₁-C₆ cycloalkylamino, C₃-C₈ cycloalkyl, or C₂-C₈ heterocyclyl.

In some embodiments, p is 1. In some embodiments, p is 2. In some embodiments, p is 3.

In some embodiments, L² is a bond. In some embodiments, L² is —C(═O)NR^{4B}—, —C₁-C₃alkylene-, —C₂-C₃alkynylene-, —NR^{4A}—(C₁-C₃alkylene)-, —NR^{4A}—(C₁-C₃alkylene)-C(═O)NR^{4B}, —O—(C₁-C₃ alkylene)-, or —O—(C₁-C₃ alkylene)-C(═O)NR^{4B}—. In some embodiments, L² is —C(═O)NH—, —CH₂—, —C≡C—, —NH—(CH₂)—, —NH—(CH₂)—C(═O)NH, —O—(CH₂)—, or —O—(CH₂)—C(═O)NH—. In some embodiments, L² is —C(═O)NR^{4B}—, —NR^{4A}—(C₁-C₃alkylene)-C(═O)NR^{4B}; or —O—(C₁-C₃ alkylene)-C(═O)NR^{4B}—. In some embodiments, L² is —C(═O)NH—, —NH—(CH₂)—C(═O)NH, or —O—(CH₂)—C(═O)NH—. In some embodiments, L² is —NR^{4A}— or —O—.

In some embodiments, L² is —NH—. In some embodiments, L² is —O—.

In some embodiments, the DDB1 binding moiety B is not connected to a ligand A and/or to a linker L¹.

In another aspect, the DDB1 ligand comprises the structure of Formula (L-II), or a pharmaceutically acceptable salt or solvate thereof:

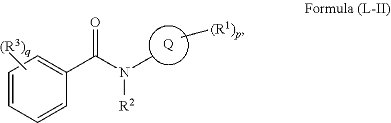

wherein,
ring Q is phenyl or a 5 or 6-membered monocyclic heteroaryl;
each R¹ is independently hydrogen, halogen, —CN, —NO₂, —OR^{4A}, —NR^{4A}R^{4B}, —C(═O)R^{4A}, —C(═O)OR^{4A}, —C(═O)NR^{4B}R^{4A}, —OC(═O)R^{4A}, —N(R^{4A})C(═O)R^{4B}, C₁-C₆ alkyl, C₁-C₆ haloalkyl, C₁-C₆ heteroalkyl, C₃-C₈ cycloalkyl, C₂-C₈ heterocyclyl, aryl or heteroaryl, or
two R¹, together with the atom(s) to which they are connected, optionally form C₃-C₁₃ cycloalkyl, C₂-C₁₂ heterocyclyl, aryl, or heteroaryl;
R² is hydrogen, C₁-C₆ alkyl, or C₃-C₈ cycloalkyl;
each R³ is independently hydrogen, halogen, —CN, —NO₂, —OR^{4A}, —NR^{4A}R^{4B}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl, or heteroaryl, or
two R³, together with the atom(s) to which they are connected, optionally form C₃-C₁₃ cycloalkyl, C₂-C₁₂ heterocyclyl, aryl, or heteroaryl;
each R^{4A} and R^{4B} is independently hydrogen, C₁-C₆ alkyl, C₂-C₆ alkenyl, C₂-C₆ alkynyl, C₁-C₆ haloalkyl, C₁-C₆ heteroalkyl, C₃-C₈ cycloalkyl, C₂-C₈ heterocyclyl, aryl, or heteroaryl, or
R^{4A} and R^{4B}, together with the atom(s) to which they are connected, optionally form C₂-C₁₂ heterocyclyl;
p is 1, 2, 3, 4 or 5; and
q is 1, 2, 3, 4, or 5.

In some embodiments, ring Q is a 5-membered monocyclic heteroaryl. In some embodiments, ring Q is a 5-membered monocyclic heteroaryl selected from pyrrolyl, furanyl, imidazolyl, pyrazolyl, oxazolyl, isoxazolyl, thienyl, thiazolyl, isothiazolyl, triazolyl, oxadiazolyl, thiadiazolyl, or tetrazolyl.

In some embodiments, the DDB1 binding moiety of Formula (L-II) has the structure of Formula (L-III-1) or (L-III-2), or a pharmaceutically acceptable salt or solvate thereof:

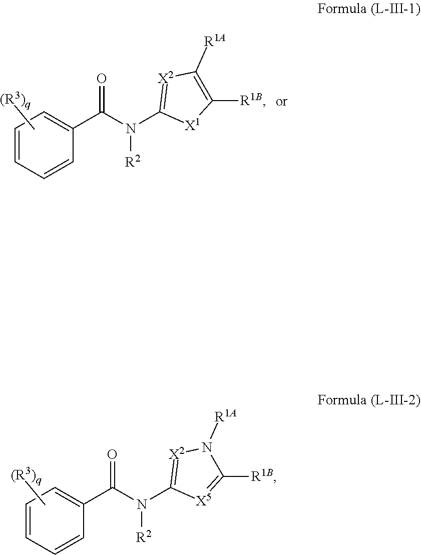

wherein,
X¹ is O, S, or NR⁵;
X² and X⁵ are independently N or CH;
R⁵ is hydrogen, C₁-C₆ alkyl, C₁-C₆ haloalkyl, C₁-C₆ heteroalkyl, C₃-C₈ cycloalkyl, C₂-C₈ heterocyclyl; and
R^{1A} and R^{1B} are independently selected from hydrogen, halogen, CN, —NO₂, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ heteroalkyl, C₁-C₆ haloalkyl, C₃-C₈ cycloalkyl, C₂-C₈ heterocyclyl, aryl or heteroaryl, or
R^{1A} and R^{1B}, together with the atom(s) to which they are connected, optionally form C₃-C₁₃ cycloalkyl, C₂-C₁₂ heterocyclyl, aryl, or heteroaryl.

In some embodiments, X¹ is O or S; and X² is N. In some embodiments, R² is H.

In some embodiments, X⁵ is CH. In some embodiments, X⁵ is N.

In some embodiments, X² is N.

In some embodiments, the DDB1 binding moiety of Formula (L-II) has the structure of Formula (L-IV-1) or (L-IV-2), or a pharmaceutically acceptable salt or solvate thereof:

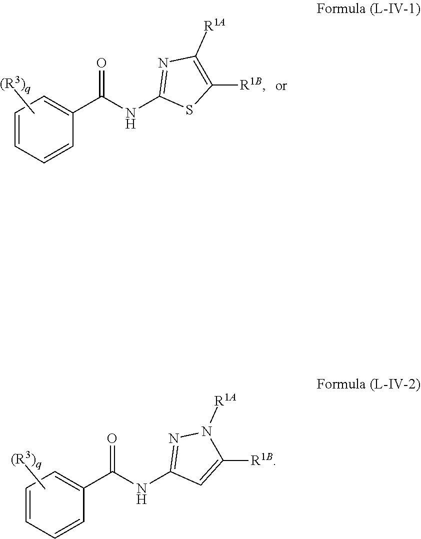

In some embodiments, R^{1A} is selected from hydrogen, halogen, —NO₂, —OCH₃, —NH₂, —NHCH₃, —N(CH₃)₂, —C(═O)CH₃, —C(═O)OCH₃, —C(═O)NH₂, —C(═O)NHCH₃, —C(═O)N(CH₃)₂, —CH₃, —CF₃, —CH₂CH₃, —CH(CH₃)₂, —C(CH₃)₃, cyclopropyl, cyclobutyl, cyclopentyl, cyclohexyl, or phenyl. In some embodiments, R^{1B} is selected from hydrogen, halogen, —NO₂, —OCH₃, —NH₂, —NHCH₃, —N(CH₃)₂, —C(═O)CH₃, —C(═O)OCH₃, —C(═O)NH₂, —C(═O)NHCH₃, —C(═O)N(CH₃)₂, —CHF₂, —CF₃, or phenyl. In some embodiments, R^{1B} is selected from —CH₃, —CH(CH₃)₂, —C(CH₃)₃, cyclopropyl, cyclobutyl, cyclopentyl, or cyclohexyl.

In some embodiments, ring Q is a phenyl or 6-membered monocyclic heteroaryl. In some embodiments, ring Q is a 6-membered monocyclic heteroaryl selected from pyridinyl, pyridazinyl, pyrazinyl, pyrimidinyl, or triazinyl.

In some embodiments, the DDB1 binding moiety of Formula (L-II) has the structure of Formula (L-V-A), or a pharmaceutically acceptable salt or solvate thereof:

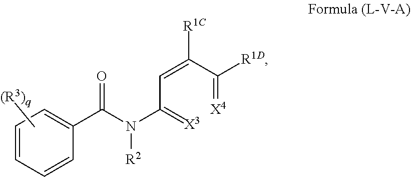

wherein,
X³ is N or CH;
X⁴ is CR^{1E} or N; and
each of R^{1C}, R^{1D}, and R^{1E} is independently selected from hydrogen, halogen, CN, —NO₂, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl or heteroaryl, or
R^{1C} and R^{1D}, or R^{1D} and R^{1E}, together with the atom(s) to which they are connected, optionally form C₃-C₁₃ cycloalkyl, C₂-C₁₂ heterocyclyl, aryl, or heteroaryl.

In some embodiment, the DDB1 binding moiety of Formula (L-II) has the structure of Formula (L-V-1) or (L-V-2), or a pharmaceutically acceptable salt or solvate thereof:

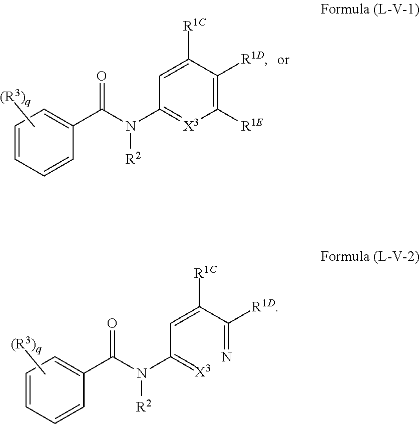

In some embodiments, R² is hydrogen. In some embodiments, X³ is N. In some embodiments, X³ is CH. In some embodiments, R^{1C} and R^{1E} are each hydrogen; and R^{1D} is hydrogen, halogen, CN, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl, or heteroaryl. In some embodiments, each R³ is independently halogen, C₁-C₆ alkyl, C₁-C₆ haloalkyl, C₁-C₆ heteroalkyl, C₁-C₆ alkoxy, C₁-C₆ alkylamino, C₁-C₆ cycloalkoxy, C₁-C₆ cycloalkylamino, C₃-C₈ cycloalkyl, or C₂-C₈ heterocyclyl. In some embodiments, R³ is C₁-C₆ alkylamino. In some embodiments, R³ is C₁-C₆ alkylamido. In some embodiments, R³ is C₁-C₆ cycloalkylamido. In some embodiments, R³ is C₁-C₆ alkyl. In some embodiments, R³ is CH₃. In some embodiments, R³ is F, Cl, Br, CH₃, CHF₂, CF₃, CH₂CH₃, CH(CH₃)₂, cyclopropyl, CN, —NH₂, NH(CH₃), NH(i-Pr), NH(n-Bu), NH(t-Bu), or N(CH₃)₂. In some embodiments, R³ is NH(CH₃). In some embodiments, p is 1, 2 or 3. In some embodiments, q is 1, 2, or 3. An R^{1D} may include —H. An R^{1D} may include —NH₂. An R^{1D} may include —NH(CH₃). An R^{1D} may include —N(CH₃)₂. An R³ may include CN, —NH₂.

In another aspect, the DDB1 ligand comprises the compounds in Table 1, or a pharmaceutically acceptable salt or solvate thereof.

In some embodiments, the binding between the DDB1 protein and the DDB1 binding moiety comprises a binding affinity with an equilibrium dissociation constant (Kd) below 100 μM, a Kd below 90 μM, a Kd below 80 μM, a Kd below 70 μM, a Kd below 60 μM, below 50 μM, a Kd below 45 μM, a Kd below 40 μM, a Kd below 35 μM, a Kd below 30 μM, a Kd below 25 μM, a Kd below 20 μM, a Kd below 15 μM, a Kd below 14 μM, a Kd below 13 μM, a Kd below 12 μM, a Kd below 11 μM, a Kd below 10 μM, a Kd below 9 μM, a Kd below 8 μM, a Kd below 7 μM, a Kd below 6 μM, a Kd below 5 μM, a Kd below 4 μM, a Kd below 3 μM, a Kd below 2 μM, or a Kd below 1 μM. In some embodiments, the binding between the DDB1 protein and the DDB1 binding moiety comprises a binding affinity with a Kd value of about 100 μM, about 90 μM, about 80 μM, about 70 μM, about 60 μM, about 50 μM, about 45 μM, about 40 μM, about 35 μM, about 30 μM, about 25 μM, about 20 μM, about 15 μM, about 14 μM, about 13 μM, about 12 μM, about 11 μM, about 10 μM, about 9 μM, about 8 μM, about 7 μM, about 6 μM, about 5 μM, about 4 μM, about 3 μM, about 2 μM, or about 1 μM, or a range of Kd values defined by any two of the aforementioned Kd values. In some embodiments, the binding between the DDB1 protein and the DDB1 binding moiety comprises a binding affinity with a Kd value of 100 μM, 90 μM, 80 μM, 70 μM, 60 μM, 50 μM, 45 μM, 40 μM, 35 μM, 30 μM, 25 μM, 20 μM, 15 μM, 14 μM, 13 μM, 12 μM, 11 μM, 10 μM, 9 μM, 8 μM, 7 μM, 6 μM, 5 μM, 4 μM, 3 μM, 2 μM, or 1 μM, or a range of Kd values defined by any two of the aforementioned Kd values.

In some embodiments, the binding between the DDB1 protein and the DDB1 binding moiety (DBM) comprises a binding affinity with a Kd below 100 μM. In some embodiments, the binding between the DDB1 protein and the DBM comprises a binding affinity with a Kd below 90 PM. In some embodiments, the binding between the DDB1 protein and the DBM comprises a binding affinity with a Kd below 80 μM. In some embodiments, the binding between the DDB1 protein and the DBM comprises a binding affinity with a Kd below 70 μM. In some embodiments, the binding between the DDB1 protein and the DBM comprises a binding affinity with a Kd below 60 μM. In some embodiments, the binding between the DDB1 protein and the DBM comprises a binding affinity with a Kd below 50 PM. In some embodiments, the binding between the DDB1 protein and the DBM comprises a binding affinity with a Kd below 45 μM. In some embodiments, the binding between the DDB1 protein and the DBM comprises a binding affinity with a Kd below 40 μM. In some embodiments, the binding between the DDB1 protein and the DBM comprises a binding affinity with a Kd below 35 μM. In some embodiments, the binding between the DDB1 protein and the DBM comprises a binding affinity with a Kd below 30 PM. In some embodiments, the binding between the DDB1 protein and the DBM comprises a binding affinity with a Kd below 25 μM. In some embodiments, the binding between the DDB1 protein and the DBM comprises a binding affinity with a Kd below 20 μM. In some embodiments, the binding between the DDB1 protein and the DBM comprises a binding affinity with a Kd below 15 μM. In some embodiments, the binding between the DDB1 protein and the DBM comprises a binding affinity with a Kd below 14 μM. In some embodiments, the binding between the DDB1 protein and the DBM comprises a binding affinity with a Kd below 13 μM. In some embodiments, the binding between the DDB1 protein and the DBM comprises a binding affinity with a Kd below 12 μM. In some embodiments, the binding between the DDB1 protein and the DBM comprises a binding affinity with a Kd below 11 μM. In some embodiments, the binding between the DDB1 protein and the DBM comprises a binding affinity with a Kd below 10 μM. In some embodiments, the binding between the DDB1 protein and the DBM comprises a binding affinity with a Kd below 9 μM. In some embodiments, the binding between the DDB1 protein and the DBM comprises a binding affinity with a Kd below 8 μM. In some embodiments, the binding between the DDB1 protein and the DBM comprises a binding affinity with a Kd below 7 μM. In some embodiments, the binding between the DDB1 protein and the DBM comprises a binding affinity with a Kd below 6 μM. In some embodiments, the binding between the DDB1 protein and the DBM comprises a binding affinity with a Kd below 5 μM. In some embodiments, the binding between the DDB1 protein and the DBM comprises a binding affinity with a Kd below 4 μM. In some embodiments, the binding between the DDB1 protein and the DBM comprises a binding affinity with a Kd below 3 μM. In some embodiments, the binding between the DDB1 protein and the DBM comprises a binding affinity with a Kd below 2 μM. In some embodiments, the binding between the DDB1 protein and the DBM comprises a binding affinity with a Kd below 1 μM.

In some embodiments, the binding between the DDB1 protein and the DDB1 binding moiety comprises a binding affinity with a Kd<20 μM, a Kd from 20-100 μM, or a Kd>100 μM. In some embodiments, the binding between the DDB1 protein and the DDB1 binding moiety comprises a binding affinity with a Kd<20 μM. In some embodiments, the binding between the DDB1 protein and the DDB1 binding moiety comprises a binding affinity with a Kd from 20-100 μM. In some embodiments, the binding between the DDB1 protein and the DDB1 binding moiety comprises a binding affinity with a Kd>100 μM.

In some embodiments, the binding between the DDB1 binding moiety and DDB1 is non-covalent. In some embodiments, the binding between the DDB1 binding moiety and DDB1 is covalent.

Disclosed herein, in some embodiments, are DDB1 binding moieties. In some embodiments, the DDB1 binding moiety binds to a DDB1 protein. In some embodiments, the DDB1 binding moiety binds to a binding region on the DDB1 protein. In some embodiments, the DDB1 binding moiety is bound to a DDB1 protein. In some embodiments, the DDB1 binding moiety is bound to a binding region on the DDB1 protein. In some embodiments, the binding region on the DDB1 protein comprises a beta propeller domain. In some embodiments, the binding region on the DDB1 protein comprises a beta propeller C (BPC) domain. In some embodiments, the binding region on the DDB1 protein comprises a top face of the BPC domain. In some embodiments, the binding region on the DDB1 protein comprises one or more of the following DDB1 protein residues: ARG327, LEU328, PRO358, ILE359, VAL360, ASP361, GLY380, ALA381, PHE382, SER720, ARG722, LYS723, SER738, ILE740, GLU787, TYR812, LEU814, SER815, ALA834, VAL836, ALA841, ALA869, TYR871, SER872, MET910, LEU912, TYR913, LEU926, TRP953, SER955, ALA956, ASN970, ALA971, PHE972, PHE1003, ASN1005, VAL1006, and/or VAL1033. In some embodiments, one or more of the following DDB1 protein residues are involved in the non-covalent binding between the DDB1 protein and the DDB1 binding moiety: ARG327, LEU328, PRO358, ILE359, VAL360, ASP361, GLY380, ALA381, PHE382, SER720, ARG722, LYS723, SER738, ILE740, GLU787, TYR812, LEU814, SER815, ALA834, VAL836, ALA841, ALA869, TYR871, SER872, MET910, LEU912, TYR913, LEU926, TRP953, SER955, ALA956, ASN970, ALA971, PHE972, PHE1003, ASN1005, VAL1006, and/or VAL1033. In some embodiments, the binding region on the DDB1 protein comprises an amino acid residue described herein, such as in the section titled “Modified Proteins.”

In some embodiments, the DDB1 binding moiety is selected from Table 1, or a pharmaceutically acceptable salt or solvate thereof.

TABLE 1
Representative DDB1 binding moieties.

| Cpd. | | |
|---|---|---|
| No. | Structure | Chemical Name |
| B1-1 | 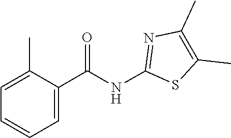 [SMILES: Cc1ccccc1C(=O)Nc1nc(C)c(C)s1] | N-(4,5-dimethylthiazol-2-yl)-2- methylbenzamide |
| B1-2 | 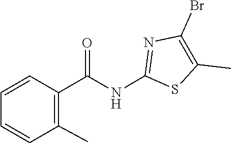 [SMILES: Cc1ccccc1C(=O)Nc1nc(Br)c(C)s1] | N-(4-bromo-5-methylthiazol-2- yl)-2-methylbenzamide |
| B1-3 | 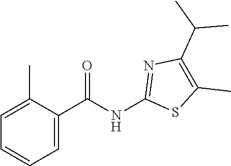 [SMILES: Cc1ccccc1C(=O)Nc1nc(C(C)C)c(C)s1] | N-(4-isopropyl-5-methylthiazol-2- yl)-2-methylbenzamide |
| B1-4 | 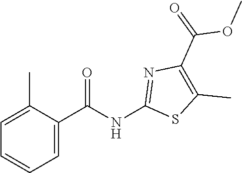 [SMILES: COC(=O)c1nc(NC(=O)c2ccccc2C)sc1C] | methyl 5-methyl-2-(2- methylbenzamido)thiazole-4- carboxylate |
| B1-5 | 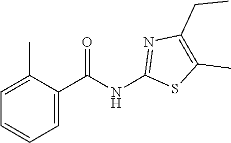 [SMILES: CCc1nc(NC(=O)c2ccccc2C)sc1C] | N-(4-ethyl-5-methylthiazol-2-yl)- 2-methylbenzamide |
| B1-6 | 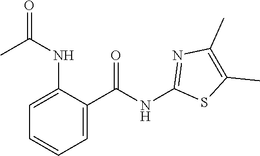 [SMILES: CC(=O)Nc1ccccc1C(=O)Nc1nc(C)c(C)s1] | 2-acetamido-N-(4,5- dimethylthiazol-2-yl)benzamide |
| B1-7 | 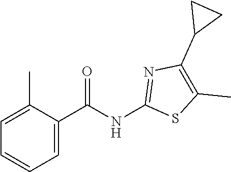 [SMILES: Cc1ccccc1C(=O)Nc1nc(C2CC2)c(C)s1] | N-(4-cyclopropyl-5- methylthiazol-2-yl)-2- methylbenzamide |
| B1-8 | 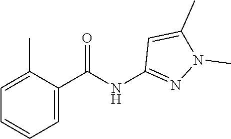 | N-(1,5-dimethyl-1H-pyrazol-3- yl)-2-methylbenzamide |
| B1-9 | 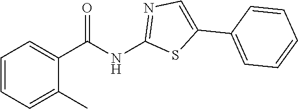 [SMILES: Cc1ccccc1C(=O)Nc1ncc(-c2ccccc2)s1] | 2-methyl-N-(5-phenylthiazol-2- yl)benzamide |
| B1-10 | 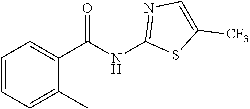 [SMILES: Cc1ccccc1C(=O)Nc1ncc(C(F)(F)F)s1] | 2-methyl-N-(5- (trifluoromethyl)thiazol-2- yl)benzamide |
| B1-11 | 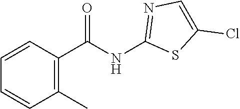 [SMILES: Cc1ccccc1C(=O)Nc1ncc(Cl)s1] | N-(5-chlorothiazol-2-yl)-2- methylbenzamide |
| B1-12 | 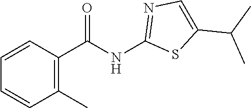 [SMILES: Cc1ccccc1C(=O)Nc1ncc(C(C)C)s1] | N-(5-isopropylthiazol-2-yl)-2- methylbenzamide |
| B1-13 | 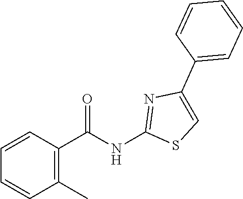 [SMILES: Cc1ccccc1C(=O)Nc1nc(-c2ccccc2)cs1] | 2-methyl-N-(4-phenylthiazol-2- yl)benzamide |
| B1-14 | 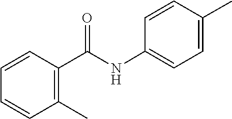 [SMILES: Cc1ccc(NC(=O)c2ccccc2C)cc1] | 2-methyl-N-(p-tolyl)benzamide |
| B1-15 | 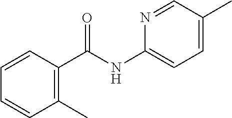 [SMILES: Cc1ccc(NC(=O)c2ccccc2C)nc1] | 2-methyl-N-(5-methylpyridin-2- yl)benzamide |
| B1-16 | 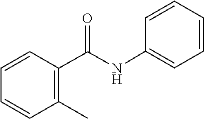 [SMILES: Cc1ccccc1C(=O)Nc1ccccc1] | 2-methyl-N-phenylbenzamide |
| B1-17 | 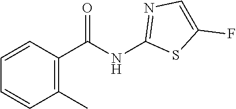 [SMILES: Cc1ccccc1C(=O)Nc1ncc(F)s1] | N-(5-fluorothiazol-2-yl)-2- methylbenzamide |
| B1-18 | 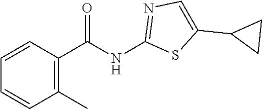 [SMILES: Cc1ccccc1C(=O)Nc1ncc(C2CC2)s1] | N-(5-cyclopropylthiazol-2-yl)-2- methylbenzamide |
| B1-19 | 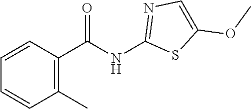 [SMILES: COc1cnc(NC(=O)c2ccccc2C)s1] | N-(5-methoxythiazol-2-yl)-2- methylbenzamide |
| B1-20 | 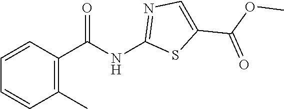 [SMILES: COC(=O)c1cnc(NC(=O)c2ccccc2C)s1] | methyl 2-(2- methylbenzamido)thiazole-5- carboxylate |
| B1-21 | 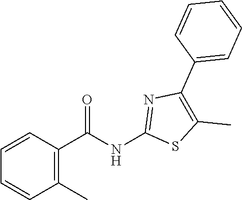 [SMILES: Cc1ccccc1C(=O)Nc1nc(-c2ccccc2)c(C)s1] | 2-methyl-N-(5-methyl-4- phenylthiazol-2-yl)benzamide |
| B1-22 | 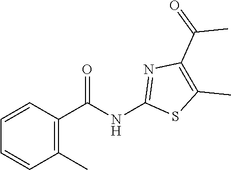 [SMILES: CC(=O)c1nc(NC(=O)c2ccccc2C)sc1C] | N-(4-acetyl-5-methylthiazol-2- yl)-2-methylbenzamide |
| B1-23 | 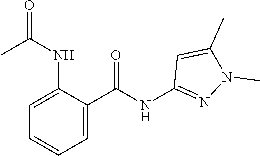 [SMILES: CC(=O)Nc1ccccc1C(=O)Nc1cc(C)n(C)n1] | 2-acetamido-N-(1,5-dimethyl-1H- pyrazol-3-yl)benzamide |
| B1-24 | 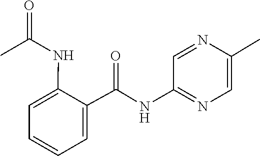 [SMILES: CC(=O)Nc1ccccc1C(=O)Nc1cnc(C)cn1] | 2-acetamido-N-(5-methylpyrazin- 2-yl)benzamide |
| B1-25 | 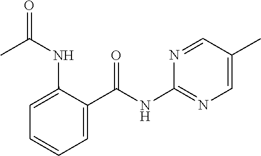 [SMILES: CC(=O)Nc1ccccc1C(=O)Nc1ncc(C)cn1] | 2-acetamido-N-(5- methylpyrimidin-2-yl)benzamide |
| B1-26 | 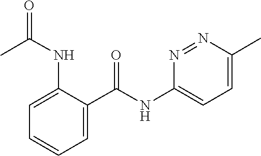 [SMILES: CC(=O)Nc1ccccc1C(=O)Nc1ccc(C)nn1] | 2-acetamido-N-(6- methylpyridazin-3-yl)benzamide |
| B1-27 | 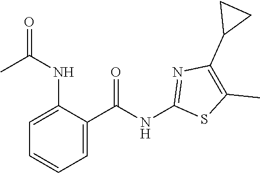 [SMILES: CC(=O)Nc1ccccc1C(=O)Nc1nc(C2CC2)c(C)s1] | 2-acetamido-N-(4-cyclopropyl-5- methylthiazol-2-yl)benzamide |
| B1-28 | 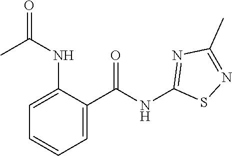 | 2-acetamido-N-(3-methyl-1,2,4- thiadiazol-5-yl)benzamide |
| B1-29 | 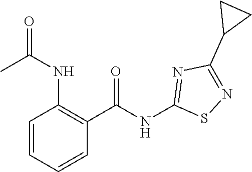 | 2-acetamido-N-(3-cyclopropyl- 1,2,4-thiadiazol-5-yl)benzamide |
| B1-30 | 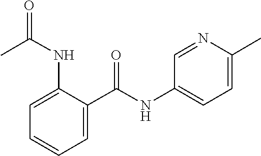 [SMILES: CC(=O)Nc1ccccc1C(=O)Nc1ccc(C)nc1] | 2-acetamido-N-(6-methylpyridin- 3-yl)benzamide |
| B1-31 | 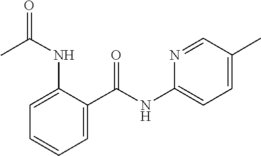 [SMILES: CC(=O)Nc1ccccc1C(=O)Nc1ccc(C)cn1] | 2-acetamido-N-(5-methylpyridin- 2-yl)benzamide |
| B1-32 | 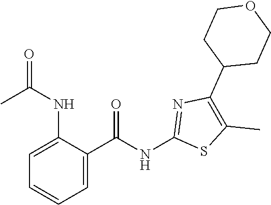 [SMILES: CC(=O)Nc1ccccc1C(=O)Nc1nc(C2CCOCC2)c(C)s1] | 2-acetamido-N-(5-methyl-4- (tetrahydro-2H-pyran-4- yl)thiazol-2-yl)benzamide |
| B1-33 | 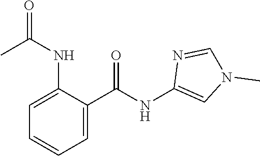 [SMILES: CC(=O)Nc1ccccc1C(=O)Nc1cn(C)cn1] | 2-acetamido-N-(1-methyl-1H- imidazol-4-yl)benzamide |
| B1-34 | 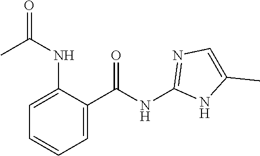 [SMILES: CC(=O)Nc1ccccc1C(=O)Nc1ncc(C)[nH]1] | 2-acetamido-N-(5-methyl-1H- imidazol-2-yl)benzamide |
| B1-35 | 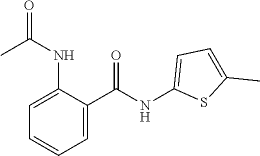 [SMILES: CC(=O)Nc1ccccc1C(=O)Nc1ccc(C)s1] | 2-acetamido-N-(5- methylthiophen-2-yl)benzamide |
| B1-36 | 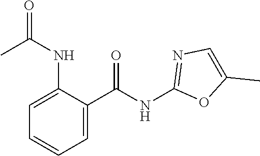 [SMILES: CC(=O)Nc1ccccc1C(=O)Nc1ncc(C)o1] | 2-acetamido-N-(5-methyloxazol- 2-yl)benzamide |
| B1-37 | 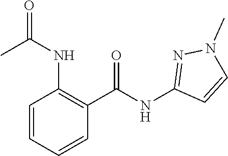 [SMILES: CC(=O)Nc1ccccc1C(=O)Nc1ccn(C)n1] | 2-acetamido-N-(1-methyl-1H- pyrazol-3-yl)benzamide |
| B1-38 | 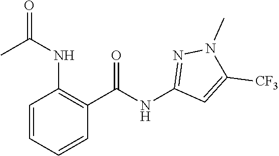 [SMILES: CC(=O)Nc1ccccc1C(=O)Nc1cc(C(F)(F)F)n(C)n1] | 2-acetamido-N-(1-methyl-5- (trifluoromethyl)-1H-pyrazol-3- yl)benzamide |
| B1-39 | 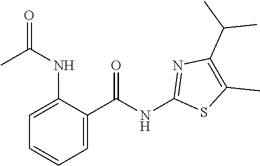 [SMILES: CC(=O)Nc1ccccc1C(=O)Nc1nc(C(C)C)c(C)s1] | 2-acetamido-N-(4-isopropyl-5- methylthiazol-2-yl)benzamide |
| B1-40 | 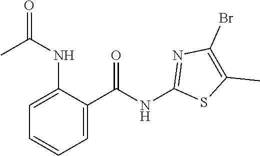 [SMILES: CC(=O)Nc1ccccc1C(=O)Nc1nc(Br)c(C)s1] | 2-acetamido-N-(4-bromo-5- methylthiazol-2-yl)benzamide |
| B1-41 | 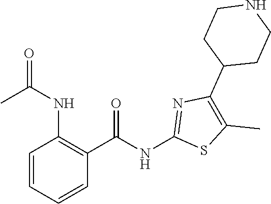 [SMILES: CC(=O)Nc1ccccc1C(=O)Nc1nc(C2CCNCC2)c(C)s1] | 2-acetamido-N-(5-methyl-4- (piperidin-4-yl)thiazol-2- yl)benzamide |
| B1-42 | 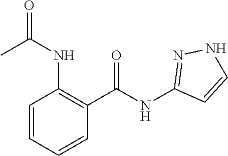 [SMILES: CC(=O)Nc1ccccc1C(=O)Nc1cc[nH]n1] | 2-acetamido-N-(1H-pyrazol-3- yl)benzamide |
| B1-43 | 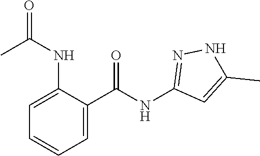 [SMILES: CC(=O)Nc1ccccc1C(=O)Nc1cc(C)[nH]n1] | 2-acetamido-N-(5-methyl-1H- pyrazol-3-yl)benzamide |
| B1-44 | 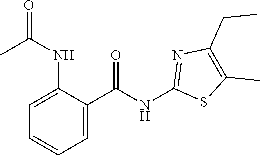 [SMILES: CCc1nc(NC(=O)c2ccccc2NC(C)=O)sc1C] | 2-acetamido-N-(4-ethyl-5- methylthiazol-2-yl)benzamide |
| B1-45 | 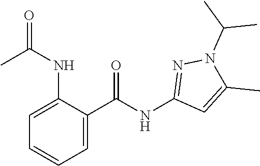 [SMILES: CC(=O)Nc1ccccc1C(=O)Nc1cc(C)n(C(C)C)n1] | 2-acetamido-N-(1-isopropyl-5- methyl-1H-pyrazol-3- yl)benzamide |
| B1-46 | 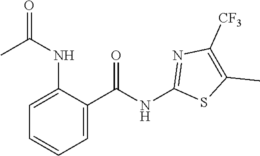 [SMILES: CC(=O)Nc1ccccc1C(=O)Nc1nc(C(F)(F)F)c(C)s1] | 2-acetamido-N-(5-methyl-4- (trifluoromethyl)thiazol-2- yl)benzamide |
| B1-47 | 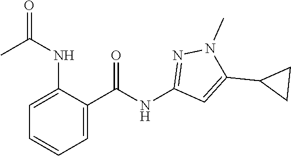 [SMILES: CC(=O)Nc1ccccc1C(=O)Nc1cc(C2CC2)n(C)n1] | 2-acetamido-N-(5-cyclopropyl-1- methyl-1H-pyrazol-3- yl)benzamide |
| B1-48 | 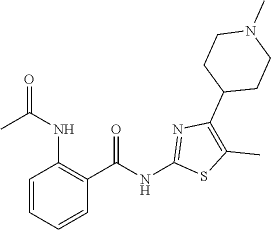 [SMILES: CC(=O)Nc1ccccc1C(=O)Nc1nc(C2CCN(C)CC2)c(C)s1] | 2-acetamido-N-(5-methyl-4-(1- methylpiperidin-4-yl)thiazol-2- yl)benzamide |
| B1-49 | 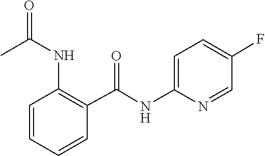 [SMILES: CC(=O)Nc1ccccc1C(=O)Nc1ccc(F)cn1] | 2-acetamido-N-(5-fluoropyridin- 2-yl)benzamide |
| B1-50 | 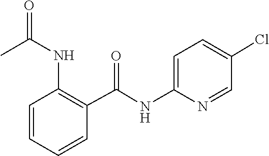 [SMILES: CC(=O)Nc1ccccc1C(=O)Nc1ccc(Cl)cn1] | 2-acetamido-N-(5-chloropyridin- 2-yl)benzamide |
| B1-51 | 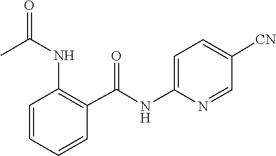 [SMILES: CC(=O)Nc1ccccc1C(=O)Nc1ccc(C#N)cn1] | 2-acetamido-N-(5-cyanopyridin- 2-yl)benzamide |
| B1-52 | 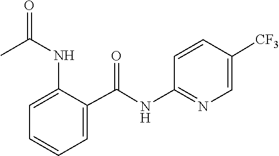 [SMILES: CC(=O)Nc1ccccc1C(=O)Nc1ccc(C(F)(F)F)cn1] | 2-acetamido-N-(5- (trifluoromethyl)pyridin-2- yl)benzamide |
| B1-53 | 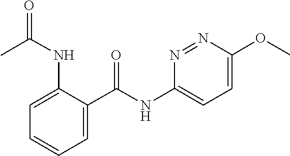 [SMILES: COc1ccc(NC(=O)c2ccccc2NC(C)=O)nn1] | 2-acetamido-N-(6- methoxypyridazin-3- yl)benzamide |
| B1-54 | 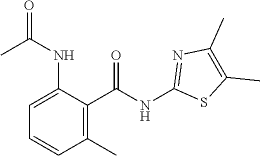 [SMILES: CC(=O)Nc1cccc(C)c1C(=O)Nc1nc(C)c(C)s1] | 2-acetamido-N-(4,5- dimethylthiazol-2-yl)-6- methylbenzamide |
| B1-55 | 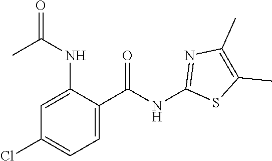 [SMILES: CC(=O)Nc1cc(Cl)ccc1C(=O)Nc1nc(C)c(C)s1] | 2-acetamido-4-chloro-N-(4,5- dimethylthiazol-2-yl)benzamide |
| B1-56 | 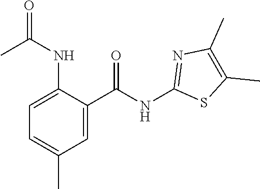 [SMILES: CC(=O)Nc1ccc(C)cc1C(=O)Nc1nc(C)c(C)s1] | 2-acetamido-N-(4,5- dimethylthiazol-2-yl)-5- methylbenzamide |
| B1-57 | 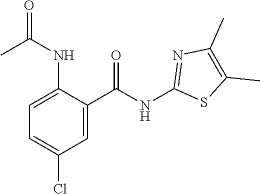 [SMILES: CC(=O)Nc1ccc(Cl)cc1C(=O)Nc1nc(C)c(C)s1] | 2-acetamido-5-chloro-N-(4,5- dimethylthiazol-2-yl)benzamide |
| B1-58 | 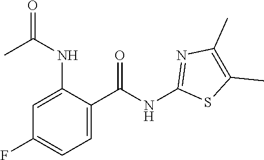 [SMILES: CC(=O)Nc1cc(F)ccc1C(=O)Nc1nc(C)c(C)s1] | 2-acetamido-N-(4,5- dimethylthiazol-2-yl)-4- fluorobenzamide |
| B1-59 | 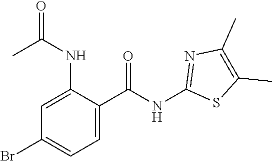 [SMILES: CC(=O)Nc1cc(Br)ccc1C(=O)Nc1nc(C)c(C)s1] | 2-acetamido-4-bromo-N-(4,5- dimethylthiazol-2-yl)benzamide |
| B1-60 | 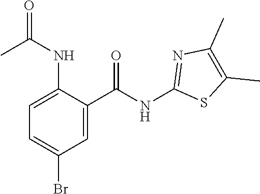 [SMILES: CC(=O)Nc1ccc(Br)cc1C(=O)Nc1nc(C)c(C)s1] | 2-acetamido-5-bromo-N-(4,5- dimethylthiazol-2-yl)benzamide |
| B1-61 | 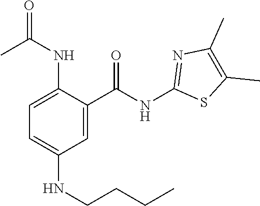 [SMILES: CCCCNc1ccc(NC(C)=O)c(C(=O)Nc2nc(C)c(C)s2)c1] | 2-acetamido-5-(butylamino)-N- (4,5-dimethylthiazol-2- yl)benzamide |
| B1-62 | 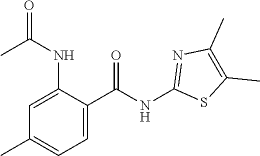 [SMILES: CC(=O)Nc1cc(C)ccc1C(=O)Nc1nc(C)c(C)s1] | 2-acetamido-N-(4,5- dimethylthiazol-2-yl)-4- methylbenzamide |
| B1-63 | 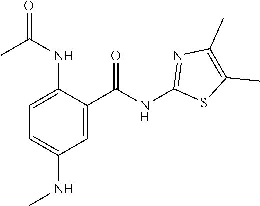 [SMILES: CNc1ccc(NC(C)=O)c(C(=O)Nc2nc(C)c(C)s2)c1] | 2-acetamido-N-(4,5- dimethylthiazol-2-yl)-5- (methylamino)benzamide |
| B1-64 | 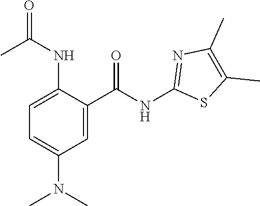 [SMILES: CC(=O)Nc1ccc(N(C)C)cc1C(=O)Nc1nc(C)c(C)s1] | 2-acetamido-5-(dimethylamino)- N-(4,5-dimethylthiazol-2- yl)benzamide |
| B1-65 | 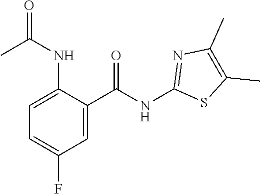 [SMILES: CC(=O)Nc1ccc(F)cc1C(=O)Nc1nc(C)c(C)s1] | 2-acetamido-N-(4,5- dimethylthiazol-2-yl)-5- fluorobenzamide |
| B1-66 | 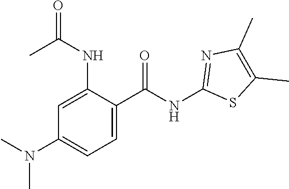 | 2-acetamido-4-(dimethylamino)- N-(4,5-dimethylthiazol-2- yl)benzamide |
| B1-67 | 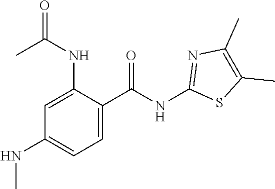 [SMILES: CNc1ccc(C(=O)Nc2nc(C)c(C)s2)c(NC(C)=O)c1] | 2-acetamido-N-(4,5- dimethylthiazol-2-yl)-4- (methylamino)benzamide |
| B1-68 | 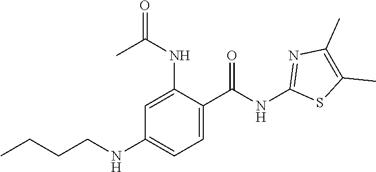 [SMILES: CCCCNc1ccc(C(=O)Nc2nc(C)c(C)s2)c(NC(C)=O)c1] | 2-acetamido-4-(butylamino)-N- (4,5-dimethylthiazol-2- yl)benzamide |
| B1-69 | 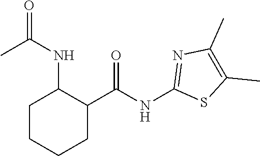 [SMILES: CC(=O)NC1CCCCC1C(=O)Nc1nc(C)c(C)s1] | 2-acetamido-N-(4,5- dimethylthiazol-2- yl)cyclohexane-1-carboxamide |
| B1-70 | 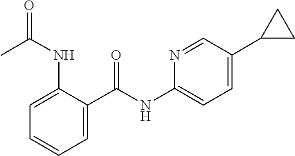 [SMILES: CC(=O)Nc1ccccc1C(=O)Nc1ccc(C2CC2)cn1] | 2-acetamido-N-(5- cyclopropylpyridin-2- yl)benzamide |
| B1-71 | 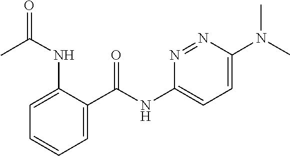 [SMILES: CC(=O)Nc1ccccc1C(=O)Nc1ccc(N(C)C)nn1] | 2-acetamido-N-(6- (dimethylamino)pyridazin-3- yl)benzamide |
| B1-72 | 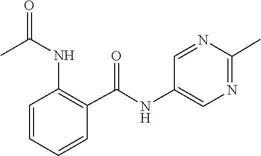 [SMILES: CC(=O)Nc1ccccc1C(=O)Nc1cnc(C)nc1] | 2-acetamido-N-(2- methylpyrimidin-5-yl)benzamide |
| B1-73 | 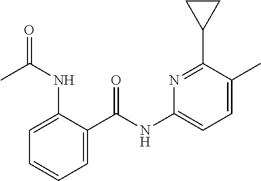 [SMILES: CC(=O)Nc1ccccc1C(=O)Nc1ccc(C)c(C2CC2)n1] | 2-acetamido-N-(6-cyclopropyl-5- methylpyridin-2-yl)benzamide |
| B1-74 | 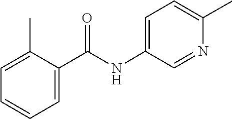 [SMILES: Cc1ccc(NC(=O)c2ccccc2C)cn1] | 2-methyl-N-(6-methylpyridin-3- yl)benzamide |
| B1-75 | 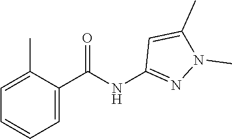 | N-(1,5-dimethyl-1H-pyrazol-3- yl)-2-methylbenzamide |
| B1-76 | 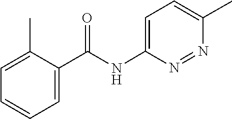 [SMILES: Cc1ccc(NC(=O)c2ccccc2C)nn1] | 2-methyl-N-(6-methylpyridazin- 3-yl)benzamide |
| B1-77 | 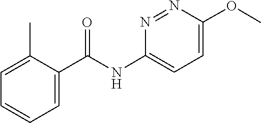 [SMILES: COc1ccc(NC(=O)c2ccccc2C)nn1] | N-(6-methoxypyridazin-3-yl)-2- methylbenzamide |
| B1-78 | 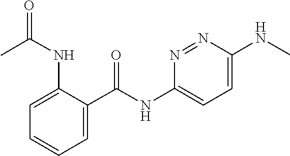 [SMILES: CNc1ccc(NC(=O)c2ccccc2NC(C)=O)nn1] | 2-acetamido-N-(6- (methylamino)pyridazin-3- yl)benzamide |
| B1-79 | 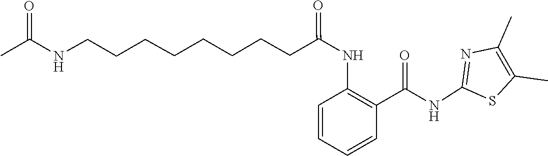 [SMILES: CC(=O)NCCCCCCCCC(=O)Nc1ccccc1C(=O)Nc1nc(C)c(C)s1] | 2-(9-acetamidononanamido)-N- (4,5-dimethylthiazol-2- yl)benzamide |
| B1-80 | 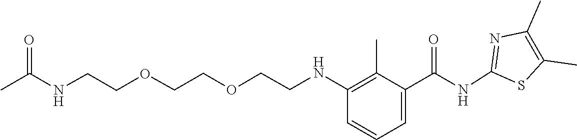 [SMILES: CC(=O)NCCOCCOCCNc1cccc(C(=O)Nc2nc(C)c(C)s2)c1C] | 3-((2-(2-(2- acetamidoethoxy)ethoxy)ethyl)amino)- N-(4,5-dimethylthiazol-2-yl)- 2-methylbenzamide |
| B1-81 | 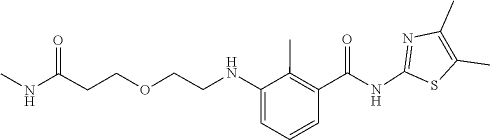 | N-(4,5-dimethylthiazol-2-yl)-2- methyl-3-((2-(3-(methylamino)-3- oxopropoxy)ethyl)amino)benzamide |
| B1-82 | 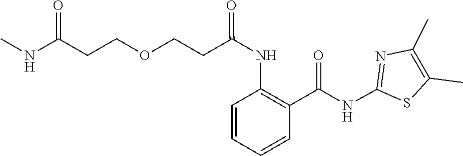 | N-(4,5-dimethylthiazol-2-yl)-2-(3- (3-(methylamino)-3- oxopropoxy)propanamido)benzamide |
| B1-83 | 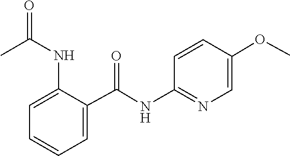 [SMILES: COc1ccc(NC(=O)c2ccccc2NC(C)=O)nc1] | 2-acetamido-N-(5- methoxypyridin-2-yl)benzamide |
| B1-84 | 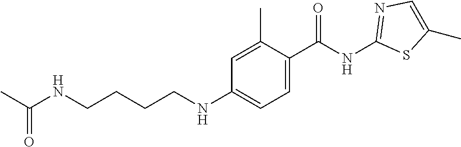 [SMILES: CC(=O)NCCCCNc1ccc(C(=O)Nc2ncc(C)s2)c(C)c1] | 2-methyl-4-(methylamino)-N-(5- methylthiazol-2-yl)benzamide |
| B1-85 | 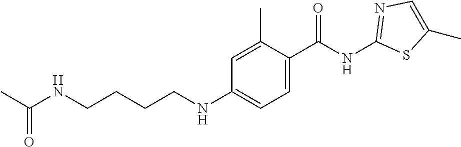 [SMILES: CC(=O)NCCCCNc1ccc(C(=O)Nc2ncc(C)s2)c(C)c1] | 4-((4-acetamidobutyl)amino)-2- methyl-N-(5-methylthiazol-2- yl)benzamide |
| B1-86 | 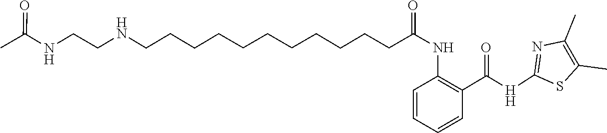 | 2-(12-((2- acetamidoethyl)amino)dodecanamido)- N-(4,5-dimethylthiazol-2- yl)benzamide |
| B1-87 | 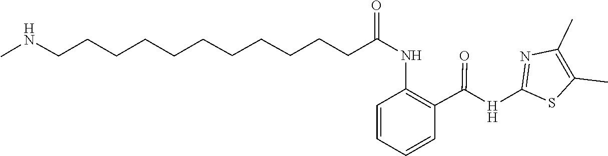 | N-(4,5-dimethylthiazol-2-yl)-2- (12- (methylamino)dodecanamido) benzamide |
| B1-88 | 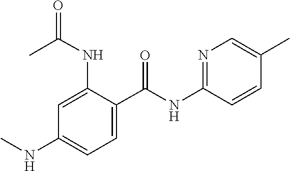 | 2-acetamido-4-(methylamino)-N- (5-methylpyridin-2-yl)benzamide |
| B1-89 | 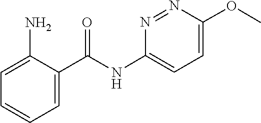 [SMILES: COc1ccc(NC(=O)c2ccccc2N)nn1] | 2-amino-N-(6-methoxypyridazin- 3-yl)benzamide |
| B1-90 | 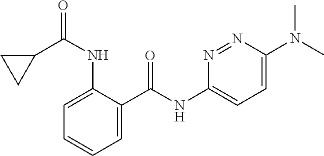 [SMILES: CN(C)c1ccc(NC(=O)c2ccccc2NC(=O)C2CC2)nn1] | 2-(cyclopropanecarboxamido)-N- (6-(dimethylamino)pyridazin-3- yl)benzamide |
| B1-91 | 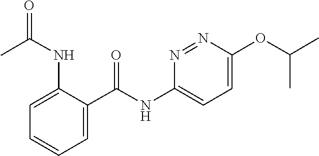 [SMILES: CC(=O)Nc1ccccc1C(=O)Nc1ccc(OC(C)C)nn1] | 2-acetamido-N-(6- isopropoxypyridazin-3- yl)benzamide |
| B1-92 | 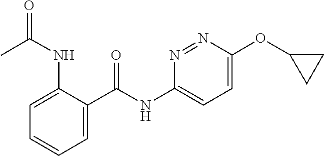 [SMILES: CC(=O)Nc1ccccc1C(=O)Nc1ccc(OC2CC2)nn1] | 2-acetamido-N-(6- cyclopropoxypyridazin-3- yl)benzamide |
| B1-93 | 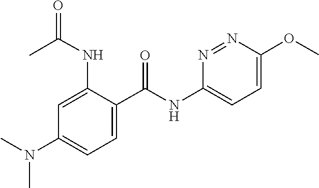 | 2-acetamido-4-(dimethylamino)- N-(6-methoxypyridazin-3- yl)benzamide |
| B1-94 | 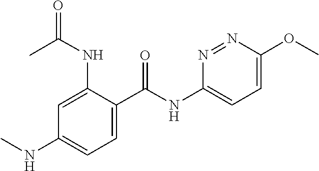 | 2-acetamido-N-(6- methoxypyridazin-3-yl)-4- (methylamino)benzamide |
| B1-95 | 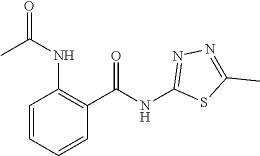 [SMILES: CC(=O)Nc1ccccc1C(=O)Nc1nnc(C)s1] | 2-acetamido-N-(5-methyl-1,3,4- thiadiazol-2-yl)benzamide |
| B1-96 | 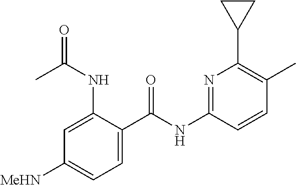 [SMILES: CNc1ccc(C(=O)Nc2ccc(C)c(C3CC3)n2)c(NC(C)=O)c1] | 2-acetamido-N-(6-cyclopropyl-5- methylpyridin-2-yl)-4- (methylamino)benzamide |
| B1-97 | 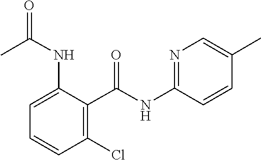 [SMILES: CC(=O)Nc1cccc(Cl)c1C(=O)Nc1ccc(C)cn1] | 2-acetamido-6-chloro-N-(5- methylpyridin-2-yl)benzamide |
| B1-98 | 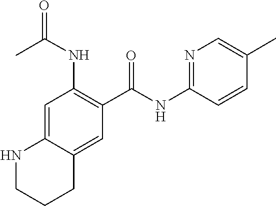 [SMILES: CC(=O)Nc1cc2c(cc1C(=O)Nc1ccc(C)cn1)CCCN2] | 7-acetamido-N-(5-methylpyridin- 2-yl)-1,2,3,4-tetrahydroquinoline- 6-carboxamide |
| B1-99 | 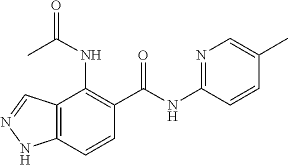 [SMILES: CC(=O)Nc1c(C(=O)Nc2ccc(C)cn2)ccc2[nH]ncc12] | 4-acetamido-N-(5-methylpyridin- 2-yl)-1H-indazole-5-carboxamide |
| B1-100 | 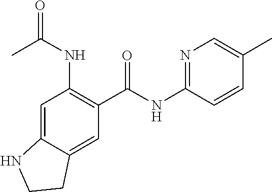 [SMILES: CC(=O)Nc1cc2c(cc1C(=O)Nc1ccc(C)cn1)CCN2] | 6-acetamido-N-(5-methylpyridin- 2-yl)indoline-5-carboxamide |
| B1-101 | 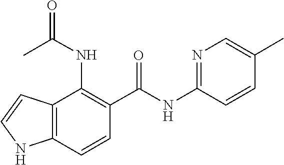 [SMILES: CC(=O)Nc1c(C(=O)Nc2ccc(C)cn2)ccc2[nH]ccc12] | 4-acetamido-N-(5-methylpyridin- 2-yl)-1H-indole-5-carboxamide |
| B1-102 | 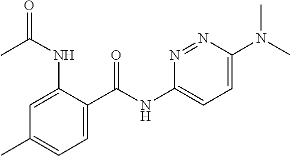 [SMILES: CC(=O)Nc1cc(C)ccc1C(=O)Nc1ccc(N(C)C)nn1] | 2-acetamido-N-(6- (dimethylamino)pyridazin-3-yl)- 4-methylbenzamide |
| B1-103 | 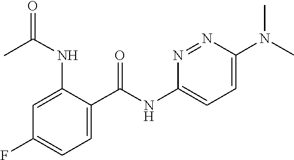 [SMILES: CC(=O)Nc1cc(F)ccc1C(=O)Nc1ccc(N(C)C)nn1] | 2-acetamido-N-(6- (dimethylamino)pyridazin-3-yl)- 4-fluorobenzamide |
| B1-104 | 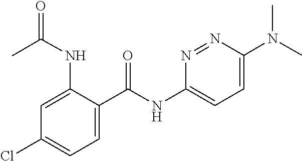 [SMILES: CC(=O)Nc1cc(Cl)ccc1C(=O)Nc1ccc(N(C)C)nn1] | 2-acetamido-4-chloro-N-(6- (dimethylamino)pyridazin-3- yl)benzamide |

### Linkers

Described herein are compounds comprising a linker. In some embodiments, the linker is connected to a DDB1 binding moiety described herein. In some embodiments, the linker is connected to a target protein binding moiety described herein. In some embodiments, the linker is connected to a DDB1 binding moiety and to a target protein binding moiety. In some embodiments, the connection is covalent. In some embodiments, the linker is incorporated into a ligand described herein.

Described herein are compounds comprising a DDB1 binding moiety and a linker. In some embodiments, the linker comprises optionally substituted polyethylene glycol (PEG). In some embodiments, the linker comprises an optionally substituted alkyl chain. In some embodiments, the linker is a straight chain alkane. In some embodiments, the linker comprises optionally substituted C₂-C₃₀, C₂-C₂₅, C₃-C₂₅, C₄-C₁₀, C₆-C₁₂, C₆-C₁₈, or C₄-C₂₀ alkyl units. In some embodiments, the linker comprises an optionally substituted carbocycle ring. In some embodiments, the linker comprises an optionally substituted heterocycle ring. In some embodiments, the linker comprises an optionally substituted aryl ring. In some embodiments, the linker comprises an optionally substituted heteroaryl ring. In some embodiments, the linker comprises ethers. In some embodiments, the linker comprises one or more C₂-C₃₀, C₂-C₂₅, C₃-C₂₅, C₄-C₁₀, C₆-C₁₂, C₆-C₁₈, or C₄-C₂₀ alkylether units. In some embodiments, the PEG is optionally substituted 1-5, 2-7, 2-10, 2-20, 5-25, or 4-30 —(O—CH₂CH₂)— units in length. In some embodiments, the linker comprises amines. In some embodiments, the linker comprises one or more C₂-C₃₀, C₂-C₂₅, C₃-C₂₅, C₄-C₁₀, C₆-C₁₂, C₆-C₁₈, or C₄-C₂₀ alkylamino units. In some embodiments, the linker comprises optionally substituted 1-5, 2-7, 2-10, 2-20, 5-25, or 4-30 —(NH—CH₂CH₂)— units. In some embodiments, the linker comprises amides. In some embodiments, the linker comprises sulfonamides. In some embodiments, the linker comprises carbamides. In some embodiments, the linker comprises carbamates. In some embodiments, the linker comprises carbonates. In some embodiments, a compound comprises a DDB1 binding moiety, a linker, and/or a target protein binding moiety.

In some embodiments, linker L¹ is a divalent moiety having the structure of Formula (L), or a pharmaceutically acceptable salt or solvate thereof:

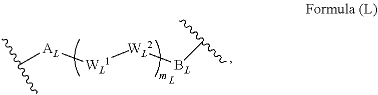

wherein,
A_{L}, W_{L}¹, W_{L}², and B_{L}, at each occurrence, is a bivalent moiety independently selected from the group consisting of a bond, R_{L}^{a}—R_{L}^{b}, R_{L}^{a}COR_{L}^{b}, R_{L}^{a}C(O)OR_{L}^{b}, R_{L}^{a}C(O)N(R_{L}¹)R_{L}^{b}, R_{L}^{a}C(S)N(R_{L}¹)R_{L}^{b}, R_{L}^{a}OR_{L}^{b}, R_{L}^{a}SR_{L}^{b}, R_{L}^{a}SOR_{L}^{b}, R_{L}^{a}SO₂R_{L}^{b}, R_{L}^{a}SO₂N(R_{L}¹)R_{L}^{b}, R_{L}^{a}N(R_{L}¹)R_{L}^{b}, R_{L}^{a}N(R_{L}¹)COR_{L}^{b}, R_{L}^{a}N(R_{L}¹)CON(R_{L}²)R_{L}^{b}, R_{L}^{a}N(R_{L}¹)C(S)R_{L}^{b}, optionally substituted C₁-C₈ alkylene, optionally substituted C₂-C₈ alkenylene, optionally substituted C₂-C₈ alkynylene, optionally substituted 1-8 membered heteroalkylene, optionally substituted 2-8 membered heteroalkenylene, optionally substituted 2-8 membered heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ cycloalkylene, optionally substituted 3-13 membered heterocyclene, optionally substituted arylene, and optionally substituted heteroarylene, wherein
each R_{L}^{a} and R_{L}^{b} is independently a bond, R_{L}^{r}, optionally substituted (C₁-C₈ alkylene)-R_{L}^{r}, optionally substituted R_{L}^{r}—(C₁-C₈ alkylene), optionally substituted (C₁-C₈ alkylene)-R_{L}^{r}—(C₁-C₈ alkylene), or a bivalent moiety comprising of optionally substituted C₁-C₈ alkylene, optionally substituted C₂-C₈ alkenylene, optionally substituted C₂-C₈ alkynylene, optionally substituted 1-8 membered heteroalkylene, optionally substituted 2-8 membered heteroalkenylene, optionally substituted 2-8 membered heteroalkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ cycloalkylene, optionally substituted 3-13 membered heterocyclene, optionally substituted arylene, or optionally substituted heteroarylene;
each R_{L}^{r} is independently selected from optionally substituted C₃-C₁₀ cycloalkylene, optionally substituted 3-10 membered heterocyclene, optionally substituted arylene, and optionally substituted heteroarylene;
each R_{L}¹ and R_{L}² are independently selected from the group consisting of hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ cycloalkyl, optionally substituted 3-10 membered heterocyclyl, optionally substituted aryl, and optionally substituted heteroaryl; or
R_{L}^{a} and R_{L}^{b}, R_{L}¹ and R_{L}², R_{L}^{a} and R_{L}¹, R_{L}^{a} and R_{L}², R_{L}^{b} and R_{L}¹, or R_{L}^{b} and R_{L}² together with the atom(s) to which they are attached optionally form a C₃-C₂₀ carbocyclyl or 3-20 membered heterocyclyl ring; and
m_{L} is an integer selected from 1 to 15.

In some embodiments, A_{L}, W_{L}¹, W_{L}², and B_{L}, at each occurrence, is a bivalent moiety independently selected from the group consisting of a bond, R_{L}^{a}—R_{L}^{b}, R_{L}^{a}COR_{L}^{b}, R_{L}^{a}C(O)OR_{L}^{b}, R_{L}^{a}C(O)N(R_{L}¹)R_{L}^{b}, R_{L}^{a}C(S)N(R_{L}¹)R_{L}^{b}, R_{L}^{a}OR_{L}^{b}, R_{L}^{a}SR_{L}^{b}, R_{L}^{a}SOR_{L}^{b}, R_{L}^{a}SO₂R_{L}^{b}, R_{L}^{a}SO₂N(R_{L}¹)R_{L}^{b}, R_{L}^{a}N(R_{L}¹)R_{L}^{b}, R_{L}^{a}N(R_{L}¹)COR_{L}^{b}, R_{L}^{a}N(R_{L}¹)CON(R_{L}²)R_{L}^{b}, R_{L}^{a}N(R_{L}¹)C(S)R_{L}^{b}, optionally substituted C₁-C₈ alkylene, optionally substituted C₂-C₈ alkenylene, optionally substituted C₂-C₈ alkynylene, optionally substituted 1-8 membered heteroalkylene, optionally substituted 2-8 membered heteroalkenylene, optionally substituted 2-8 membered heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ cycloalkylene, optionally substituted 3-13 membered heterocyclene, optionally substituted arylene, and optionally substituted heteroarylene.

In some embodiments, A_{L}, W_{L}¹, W_{L}², and B_{L}, at each occurrence, is a bivalent moiety independently selected from the group consisting of a bond, R_{L}^{a}—R_{L}^{b}, R_{L}^{a}COR_{L}^{b}, R_{L}^{a}C(O)OR_{L}^{b}, R_{L}^{a}C(O)N(R_{L}¹)R_{L}^{b}, R_{L}^{a}C(S)N(R_{L}¹)R_{L}^{b}, R_{L}^{a}OR_{L}^{b}, R_{L}^{a}SR_{L}^{b}, R_{L}^{a}SOR_{L}^{b}, R_{L}^{a}SO₂R_{L}^{b}, R_{L}^{a}SO₂N(R_{L}¹)R_{L}^{b}, R_{L}^{a}N(R_{L}¹)R_{L}^{b}, R_{L}^{a}N(R_{L}¹)COR_{L}^{b}, R_{L}^{a}N(R_{L}¹)CON(R_{L}²)R_{L}^{b}, or R_{L}^{a}N(R_{L}¹)C(S)R_{L}^{b}. In some embodiments, A_{L}, W_{L}¹, W_{L}², and B_{L}, at each occurrence, is a bivalent moiety independently selected from the group consisting optionally substituted C₁-C₈ alkylene, optionally substituted C₂-C₈ alkenylene, optionally substituted C₂-C₈ alkynylene, optionally substituted 1-8 membered heteroalkylene, optionally substituted 2-8 membered heteroalkenylene, optionally substituted 2-8 membered heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ cycloalkylene, optionally substituted 3-13 membered heterocyclene, optionally substituted arylene, and optionally substituted heteroarylene. In some embodiments, A_{L}, W_{L}¹, W_{L}², and B_{L}, at each occurrence, is independently selected from the group consisting optionally substituted C₁-C₈ alkylene. In some embodiments, A_{L}, W_{L}¹, W_{L}², and B_{L}, at each occurrence, is independently selected from the group consisting optionally substituted C₂-C₈ alkenylene. In some embodiments, A_{L}, W_{L}¹, W_{L}², and B_{L}, at each occurrence, is independently selected from the group consisting of an optionally substituted 1-8 membered heteroalkylene. In some embodiments, A_{L}, W_{L}¹, W_{L}², and B_{L}, at each occurrence, is independently selected from the group consisting of an optionally substituted 2-8 membered heteroalkenylene. In some embodiments, A_{L}, W_{L}¹, W_{L}², and B_{L}, at each occurrence, is independently selected from the group consisting of an optionally substituted 2-8 membered heteroalkynylene. In some embodiments, A_{L}, W_{L}¹, W_{L}², and B_{L}, at each occurrence, is independently selected from the group consisting of an optionally substituted C₁-C₈alkoxyC₁-C₈alkylene. In some embodiments, A_{L}, W_{L}¹, W_{L}², and B_{L}, at each occurrence, is independently selected from the group consisting of an optionally substituted C₁-C₈ haloalkylene. In some embodiments, A_{L}, W_{L}¹, W_{L}², and B_{L}, at each occurrence, is independently selected from the group consisting of an optionally substituted C₁-C₈ hydroxyalkylene. In some embodiments, A_{L}, W_{L}¹, W_{L}², and B_{L}, at each occurrence, is independently selected from the group consisting of an optionally substituted C₃-C₁₃ cycloalkylene. In some embodiments, A_{L}, W_{L}¹, W_{L}², and B_{L}, at each occurrence, is independently selected from the group consisting of an optionally substituted 3-13 membered heterocyclene.

In some embodiments, each R_{L}^{a} and R_{L}^{b} is independently R_{L}^{r}, optionally substituted (C₁-C₈ alkylene)-R_{L}^{r}, optionally substituted R_{L}^{r}—(C₁-C₈ alkylene), optionally substituted (C₁-C₈ alkylene)-R_{L}^{r}—(C₁-C₈ alkylene), or a bivalent moiety comprising of optionally substituted C₁-C₈ alkylene, optionally substituted C₂-C₈ alkenylene, optionally substituted C₂-C₈ alkynylene, optionally substituted 1-8 membered heteroalkylene, optionally substituted 2-8 membered heteroalkenylene, optionally substituted 2-8 membered heteroalkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ cycloalkylene, optionally substituted 3-13 membered heterocyclene, optionally substituted arylene, or optionally substituted heteroarylene. In some embodiments, each R_{L}^{a} and R_{L}^{b} is independently a bond, R_{L}^{r}, optionally substituted (C₁-C₈ alkylene)-R_{L}^{r}, optionally substituted R_{L}^{r}—(C₁-C₈ alkylene), optionally substituted (C₁-C₈ alkylene)-R_{L}^{r}—(C₁-C₈ alkylene). In some embodiments, each R_{L}^{a} and R_{L}^{b} is independently selected from a bivalent moiety comprising of optionally substituted C₁-C₈ alkylene, optionally substituted C₂-C₈ alkenylene, optionally substituted C₂-C₈ alkynylene, optionally substituted 1-8 membered heteroalkylene, optionally substituted 2-8 membered heteroalkenylene, optionally substituted 2-8 membered heteroalkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ cycloalkylene, optionally substituted 3-13 membered heterocyclene, optionally substituted arylene, or optionally substituted heteroarylene.

In some embodiments, A_{L} is a bond, —C(═O)—, —C(═O)NH—, —NH—, —NH—C(═O)—, —O—, —(C₁-C₈ alkylene)-C(═O)NH—, —(C₁-C₈ alkylene)-C(═O)—, —(C₁-C₈ alkylene)NH—, —(C₁-C₈ alkylene)-NH—C(═O)—, —(C₁-C₈ alkylene)-O—, —C₁-C₈ alkylene-, or —C₂-C₈ alkynylene-. In some embodiments, A_{L} is a bond, —(C₁-C₈ alkylene)-C(═O)NH—, —(C₁-C₈ alkylene)-C(═O)—, —(C₁-C₈ alkylene)NH—, —(C₁-C₈ alkylene)-NH—C(═O)—, —(C₁-C₈ alkylene)-O—, or —C₁-C₈ alkylene-. In some embodiments, A_{L} is a bond. In some embodiments, A_{L} is —C(═O)—. In some embodiments, A_{L} is —C(═O)NH—. In some embodiments, A_{L} is —NH—. In some embodiments, A_{L} is —NH—C(═O)—. In some embodiments, A_{L} is —O—. In some embodiments, A_{L} is —(C₁-C₈ alkylene)-C(═O)NH—. In some embodiments, A_{L} is —(C₁-C₈ alkylene)-C(═O)—. In some embodiments, A_{L} is —(C₁-C₈ alkylene)NH—. In some embodiments, A_{L} is —(C₁-C₈ alkylene)-NH—C(═O)—. In some embodiments, A_{L} is —(C₁-C₈ alkylene)-O—. In some embodiments, A_{L} is —C₁-C₈ alkylene-. In some embodiments, A_{L} is —C₂-C₈ alkynylene-.

In some embodiments, B_{L} is a bond, —C(═O)—, —C(═O)NH—, —NH—, —NH—C(═O)—, —O—, —(C₁-C₈ alkylene)-, —C₂-C₈ alkynylene-, —NH—(C₁-C₈ alkylene)-, —O—(C₁-C₈ alkylene)-, —C(═O)—(C₁-C₈ alkylene)-, —C(═O)NH—(C₁-C₈ alkylene)-, or —NH—C(═O)—(C₁-C₈ alkylene)-. In some embodiments, B_{L} is a bond, —(C₁-C₈ alkylene)-, —NH—(C₁-C₈ alkylene)-, —O—(C₁-C₈ alkylene)-, —C(═O)—(C₁-C₈ alkylene)-, —C(═O)NH—(C₁-C₈ alkylene)-, or —NH—C(═O)—(C₁-C₈ alkylene)-.

In some embodiments, B_{L} is a bond. In some embodiments, B_{L} is —C(═O)—. In some embodiments, B_{L} is —C(═O)NH—. In some embodiments, B_{L} is —NH—. In some embodiments, B_{L} is —NH—C(═O)—. In some embodiments, B_{L} is —O—. In some embodiments, B_{L} is —(C₁-C₈ alkylene)-. In some embodiments, B_{L} is —C₂-C₈ alkynylene-. In some embodiments, B_{L} is —NH—(C₁-C₈ alkylene)-. In some embodiments, B_{L} is —O—(C₁-C₈ alkylene)-. In some embodiments, B_{L} is —C(═O)—(C₁-C₈ alkylene)-. In some embodiments, B_{L} is —C(═O)NH—(C₁-C₈ alkylene)-. In some embodiments, B_{L} is —NH—C(═O)—(C₁-C₈ alkylene)-.

In some embodiments, each W_{L}¹ is independently R_{L}^{r} or C₁-C₃ alkylene; and each W_{L}² is independently a bond, O, or NH. In some embodiments, each W_{L}¹ is independently C₁, C₂ or C₃ alkylene; and each W_{L}² is independently a bond, O, or NH. In some embodiments, each W_{L}¹ is independently C₁, C₂ or C₃ alkylene; and each W_{L}² is independently O or NH. In some embodiments, each W_{L}¹ is independently C₁, C₂ or C₃ alkylene; and each W_{L}² is independently O. In some embodiments, each W_{L}¹ is independently C₁, C₂ or C₃ alkylene; and each W_{L}² is independently NH.

In some embodiments, each W_{L}¹ is independently a bond, O, or NH; and each W_{L}² is independently R_{L}^{r} or C₁-C₃ alkylene. In some embodiments, each W_{L}¹ is independently a bond, O, or NH; and each W_{L}² is independently C₁, C₂ or C₃ alkylene. In some embodiments, each W_{L}¹ is independently a bond or O; and each W_{L}² is independently C₁, C₂ or C₃ alkylene. In some embodiments, each W_{L}¹ is independently O; and each W_{L}² is independently C₁, C₂ or C₃ alkylene. In some embodiments, each W_{L}¹ is independently NH; and each W_{L}² is independently C₁, C₂ or C₃ alkylene.

In some embodiments, each —W_{L}¹—W_{L}²— is independently —CH₂CH₂O— or —CH₂—. In some embodiments, each —W_{L}¹—W_{L}²— is independently —CH₂CH₂O—. In some embodiments, each —W_{L}¹—W_{L}²— is independently —CH₂—.

In some embodiments, each R_{L}^{r} is independently selected from optionally substituted C₃-C₁₀ cycloalkylene or optionally substituted 3-10 membered heterocyclene.

In some embodiments, each R_{L}^{r} is independently selected from optionally substituted C₃-C₁₀ cycloalkylene. In some embodiments, each R_{L}^{r} is independently selected from optionally substituted C₃-C₈ cycloalkylene. In some embodiments, each R_{L}^{r} is independently selected from optionally substituted C₄-C₆ cycloalkylene. In some embodiments, each R_{L}^{r} is independently selected from optionally substituted 3-10 membered heterocyclene. In some embodiments, each R_{L}^{r} is independently selected from optionally substituted 3-8 membered heterocyclene. In some embodiments, each R_{L}^{r} is independently selected from optionally substituted 4-6 membered heterocyclene. In some embodiments, each R_{L}^{r} is independently selected from optionally substituted arylene. In some embodiments, each R_{L}^{r} is independently selected from optionally substituted heteroarylene.

In some embodiments, m_{L} is selected from 1-14, 1-13, 1-12, 1-11, 1-10, 1-9, 1-8, 1-7, 1-6, 1-5, 1-4, 1-3, or 1-2. In some embodiments, m_{L} is selected from 1-13. In some embodiments, m_{L} is selected from 1-12. In some embodiments, m_{L} is selected from 1-11. In some embodiments, m_{L} is selected from 1-10. In some embodiments, m_{L} is selected from 1-9. In some embodiments, m_{L} is selected from 1-8. In some embodiments, m_{L} is selected from 1-7. In some embodiments, m_{L} is selected from 1-6. In some embodiments, m_{L} is selected from 1-5. In some embodiments, m_{L} is selected from 1-4. In some embodiments, m_{L} is selected from 1-3. In some embodiments, m_{L} is 1, 2, 3, 4, 5, 6, 7, 8, 9, 10, 11, 12, 13, 14, or 15.

In some embodiments, the linker L¹ comprises one or more rings selected from the group consisting of Formula (L-1), Formula (L-2), Formula (L-3), Formula (L-4) and Formula (L-5):

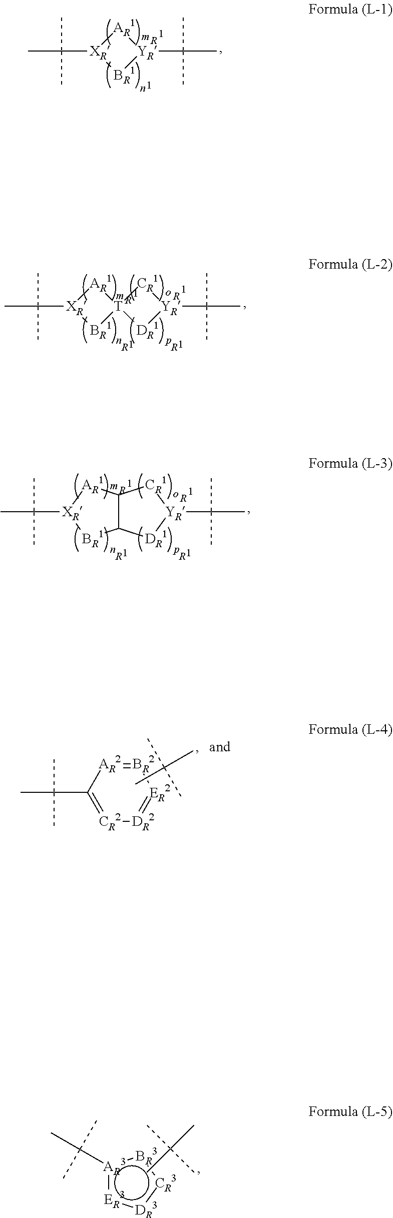

wherein
X_{R}′ and Y_{R}′ are independently selected from N, CR_{R}^{b};
A_{R}¹, B_{R}¹, C_{R}¹ and D_{R}¹, at each occurrence, are independently selected from null, O, CO, SO, SO₂, NR_{R}^{b}, and CR_{R}^{b}R_{R}^{c};
A_{R}², B_{R}², C_{R}², D_{R}², and E_{R}², at each occurrence, are independently selected from N, and CR_{R}^{b};
A_{R}³, B_{R}³, C_{R}³, D_{R}³, and E_{R}³, at each occurrence, are independently selected from N, O, S, NR_{R}^{b}, and CR_{R}^{b};
R_{R}^{b} and R_{R}^{c}, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-8 membered heterocyclyl, optionally substituted aryl, and optionally substituted heteroaryl; and
m_{R}¹, n_{R}¹, o_{R}¹ and P_{R}¹ are independently selected from 0, 1, 2, 3, 4 and 5.

In some embodiments, the linker L¹ comprises one or more rings selected from the group consisting of Formula (L-1′), Formula (L-2′), Formula (L-3′), Formula (L-4′) and Formula (L-5′):

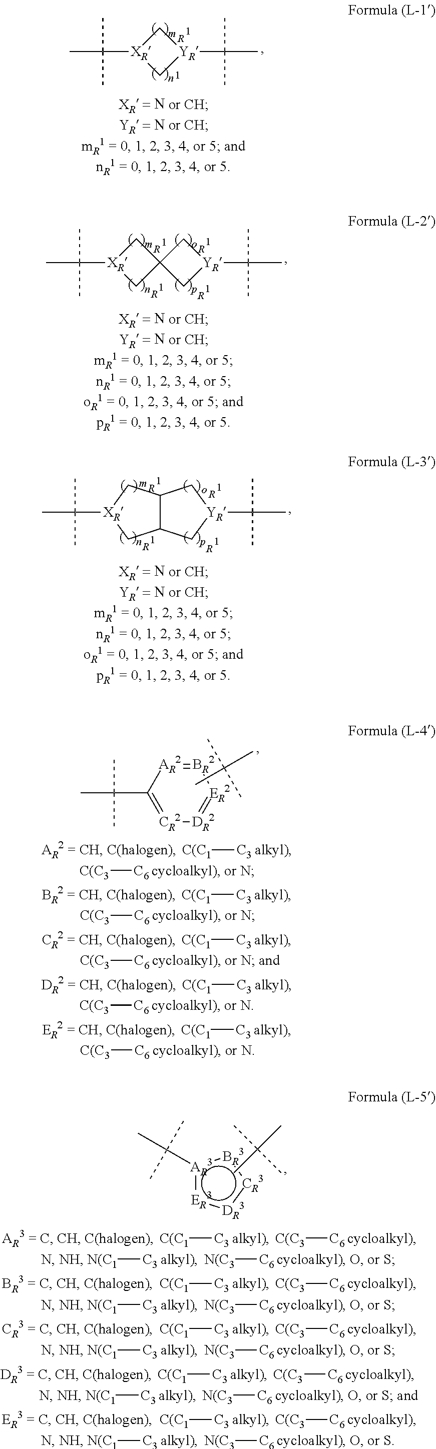

In some embodiments, the linker L¹ comprises one or more rings selected from:

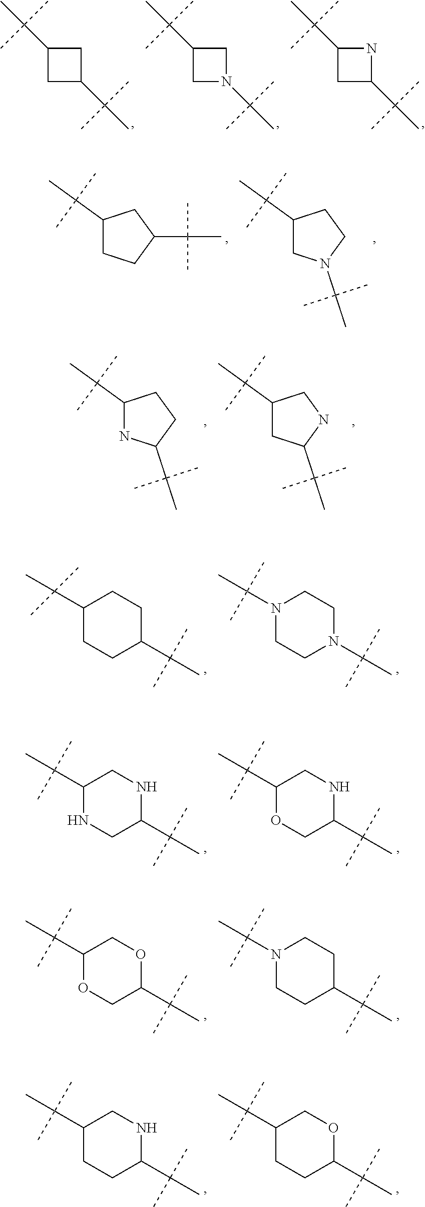

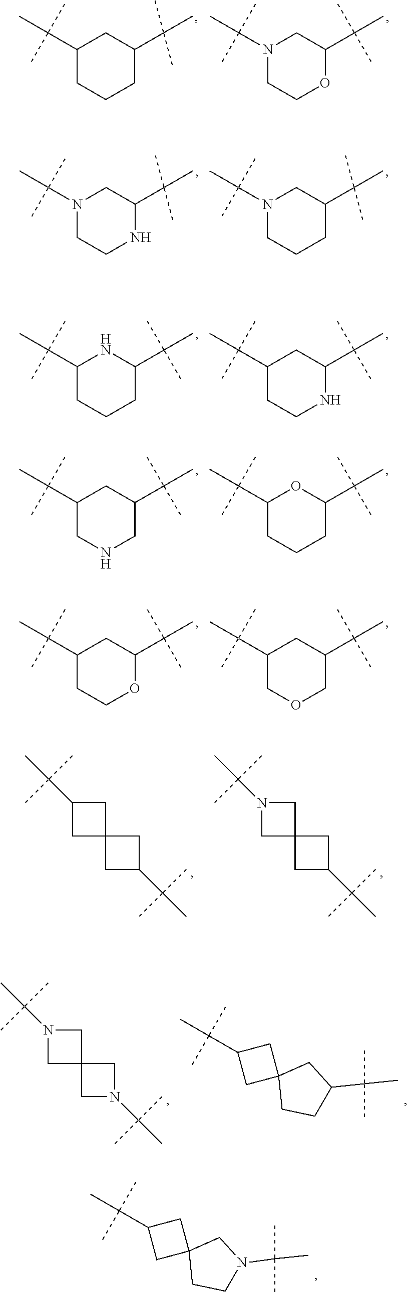

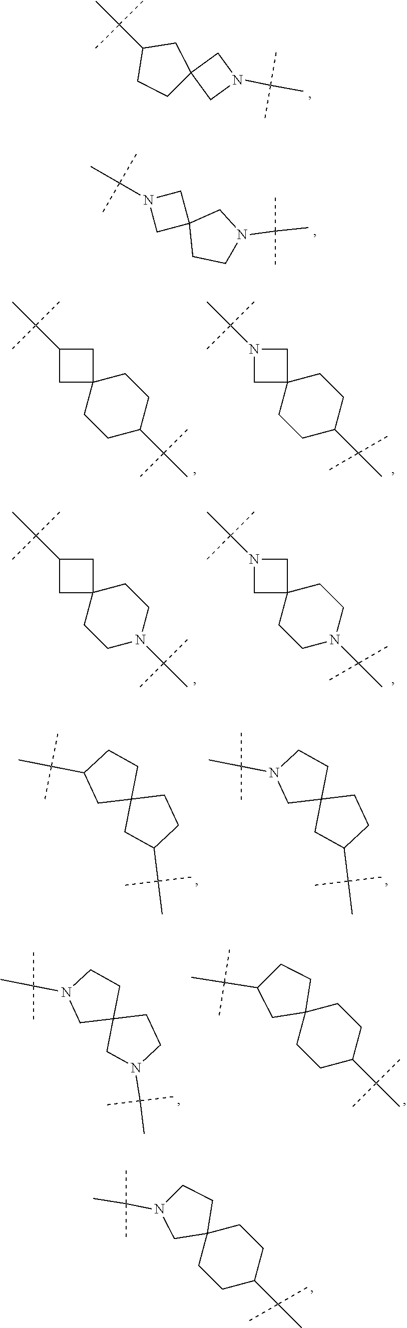

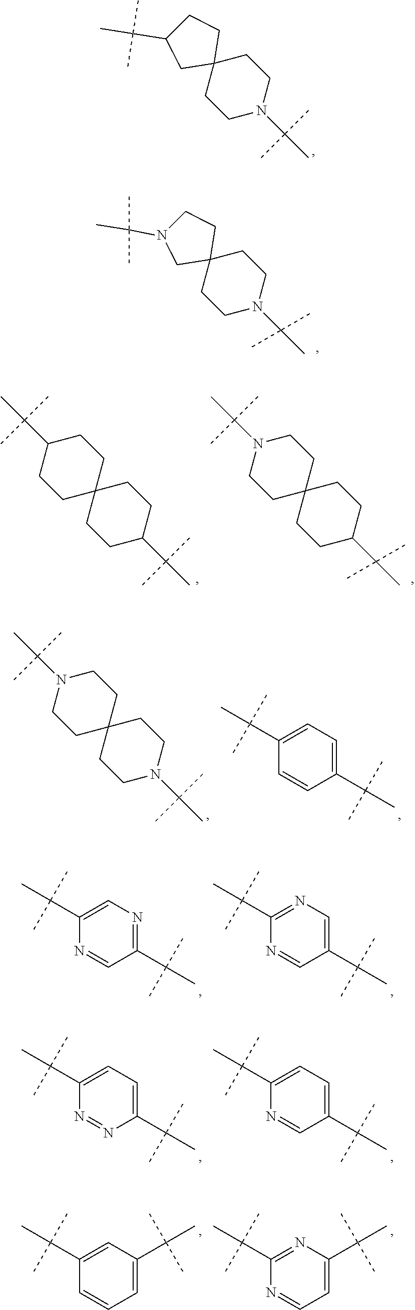

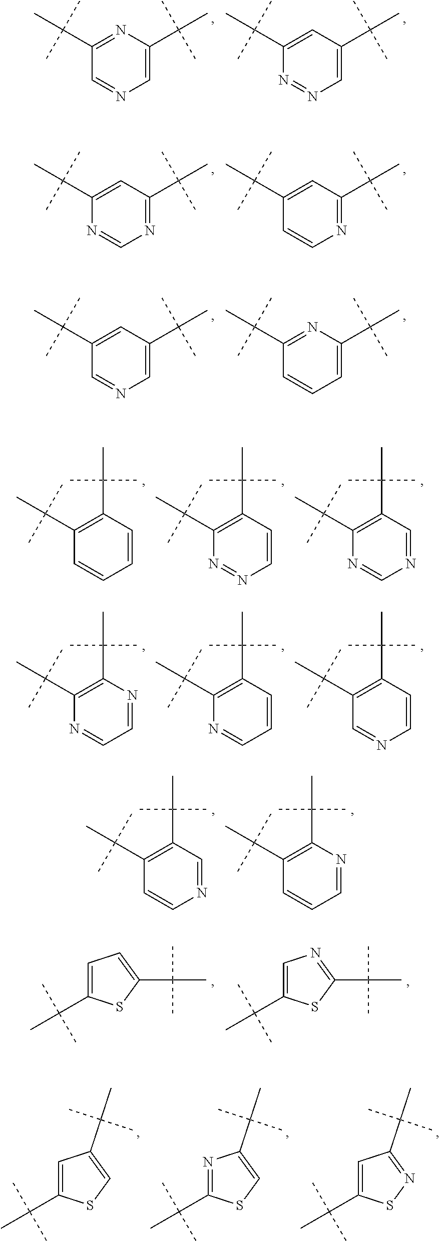

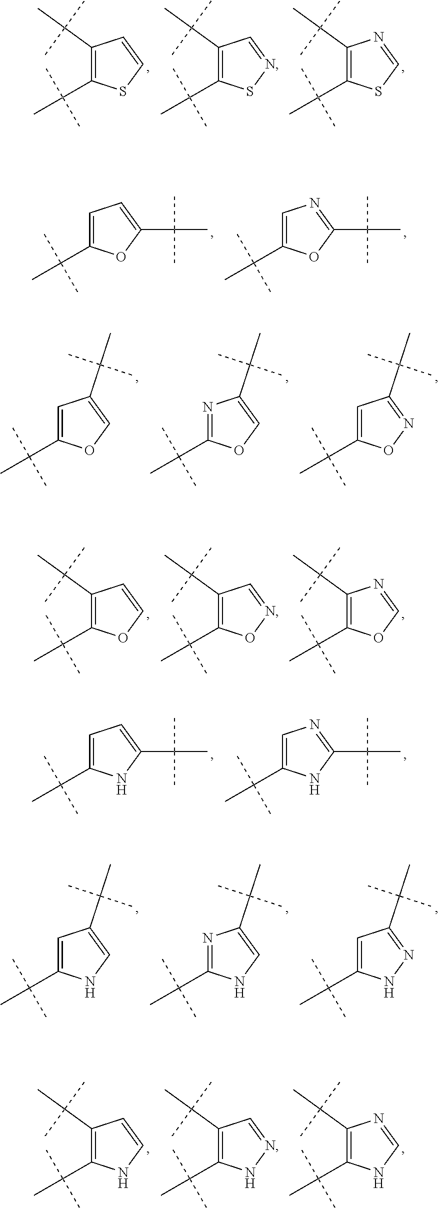

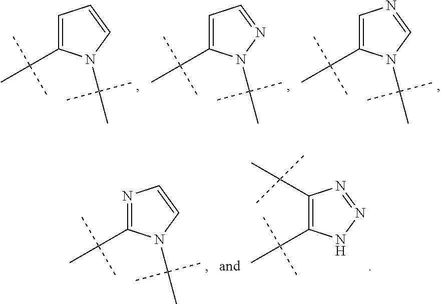

In some embodiments, the linker L¹ comprises one or more rings selected from:

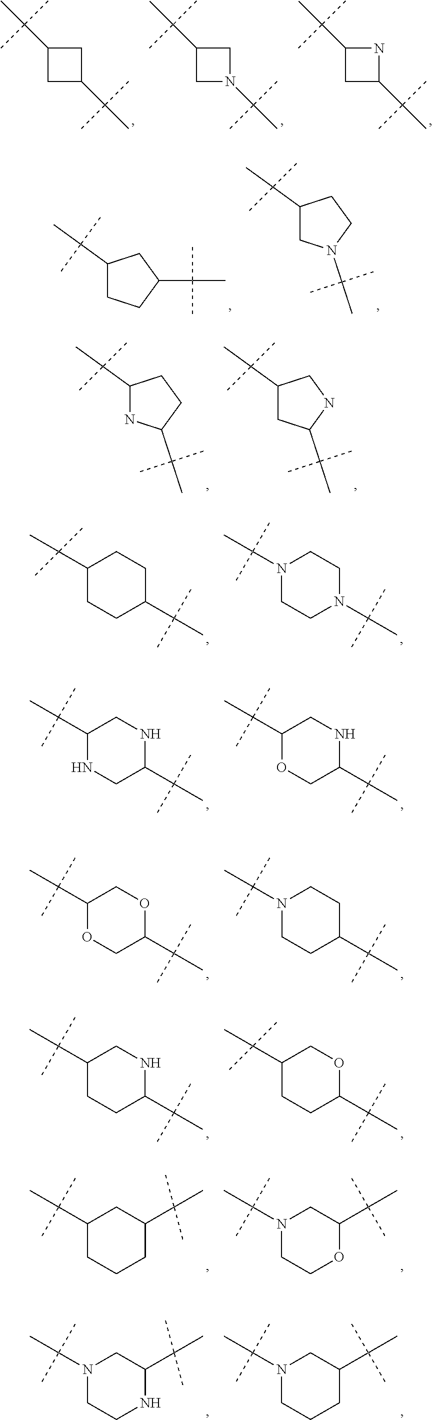

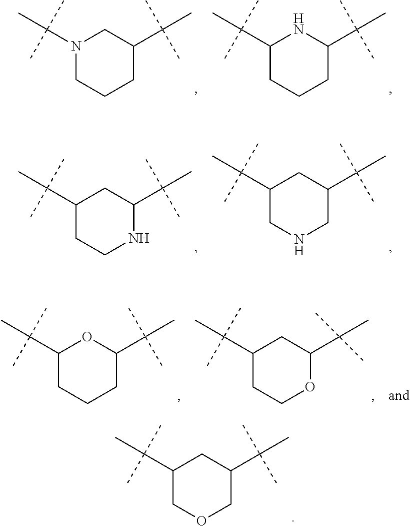

In some embodiments the linker L¹ comprises one or more rings selected from:

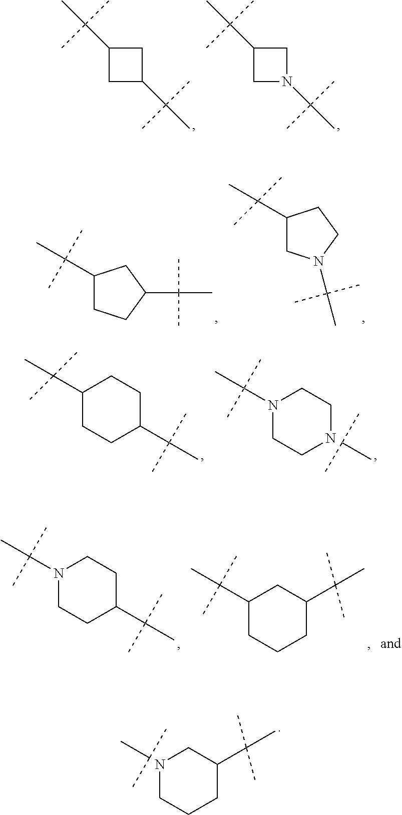

In some embodiments, the linker L¹ comprises one or more rings selected from:

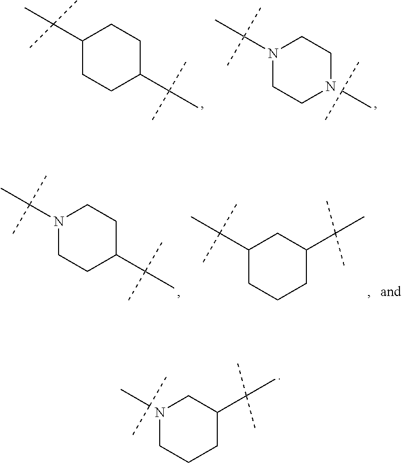

In some embodiments, the linker L¹ is —(CH₂)ₚ₁C(═O)NH(CH₂CH₂O)ₚ₂—(CH₂)ₚ₃—, —(CH₂)ₚ₁C(═O)NH(CH₂)ₚ₂—, —(CH₂)ₚ₁NHC(═O)—(CH₂CH₂O)ₚ₂—(CH₂)ₚ₃—, —(CH₂)ₚ₁NHC(═O)—(CH₂)ₚ₂—, —(CH₂)ₚ₁C(═O)—(CH₂CH₂O)ₚ₂—(CH₂)ₚ₃—, —(CH₂)ₚ₁C(═O)—(CH₂)ₚ₂—, —(CH₂)ₚ₁NH(CH₂CH₂O)ₚ₂—(CH₂)ₚ₃—, —(CH₂)ₚ₁NH(CH₂)ₚ₂—, —(CH₂CH₂O)ₚ₂—(CH₂)ₚ₃—, or —(CH₂)ₚ₂—; wherein p1 is an integer selected from 0 to 8; p2 is an integer selected from 1 to 15; and p3 is an integer selected from 0 to 8. In some embodiments, the linker L¹ is —(CH₂)ₚ₁C(═O)NH(CH₂CH₂O)ₚ₂—(CH₂)ₚ₃—, —(CH₂)ₚ₁C(═O)NH(CH₂)ₚ₂—, —(CH₂)ₚ₁NH(CH₂CH₂O)ₚ₂—(CH₂)ₚ₃—, —(CH₂)ₚ₁NH(CH₂)ₚ₂—, —(CH₂)ₚ₁C(═O)—(CH₂CH₂O)ₚ₂—(CH₂)ₚ₃—, —(CH₂)ₚ₁C(═O)—(CH₂)ₚ₂—, —(CH₂CH₂O)ₚ₂—(CH₂)ₚ₃—, or —(CH₂)ₚ₂—; wherein p1 is an integer selected from 0 to 8; p2 is an integer selected from 1 to 15; and p3 is an integer selected from 0 to 8. In some embodiments, the linker L¹ is —(CH₂)ₚ₁C(═O)NH(CH₂CH₂O)ₚ₂—(CH₂)ₚ₃—, —(CH₂)ₚ₁C(═O)NH(CH₂)ₚ₂—, —(CH₂)ₚ₁NH(CH₂CH₂O)ₚ₂—(CH₂)ₚ₃—, —(CH₂)ₚ₁C(═O)—(CH₂CH₂O)ₚ₂—(CH₂)ₚ₃—, or —(CH₂)ₚ₁C(═O)—(CH₂)ₚ₂—; wherein p1 is an integer selected from 0 to 8; p2 is an integer selected from 1 to 15; and p3 is an integer selected from 0 to 8. In some embodiments, the linker is —(CH₂)ₚ₁C(═O)NH(CH₂CH₂O)ₚ₂—(CH₂)ₚ₃—. In some embodiments, the linker is (CH₂)ₚ₁NHC(═O)—(CH₂CH₂O)ₚ₂—(CH₂)ₚ₃—. In some embodiments, the linker is (CH₂)ₚ₁NHC(═O)—(CH₂CH₂O)ₚ₂—(CH₂)ₚ₃—. In some embodiments, the linker is —(CH₂)ₚ₁NHC(═O)—(CH₂)ₚ₂—. In some embodiments, the linker is —(CH₂)ₚ₁C(═O)—(CH₂CH₂O)ₚ₂—(CH₂)ₚ₃—. In some embodiments, the linker is —(CH₂)ₚ₁C(═O)—(CH₂)ₚ₂—. In some embodiments, the linker is —(CH₂)ₚ₁NH(CH₂CH₂O)ₚ₂—(CH₂)ₚ₃—. In some embodiments, the linker is —(CH₂)ₚ₁NH(CH₂)ₚ₂—. In some embodiments, the linker is —(CH₂CH₂O)ₚ₂—(CH₂)ₚ₃—. In some embodiments, the linker is —(CH₂)ₚ₂—.

In some embodiments, the linker L¹ is —C(═O)—(CH₂)₁₋₈—, —(CH₂)₁₋₉—, —(CH₂)₁₋₂—C(═O)—NH—(CH₂)₂₋₉—, —(CH₂)₁₋₂—C(═O)—NH—(CH₂)₁₋₃—(OCH₂CH₂)₁₋₇—, —(CH₂)₀₋₁—C(═O)—(CH₂)₁₋₃—(OCH₂CH₂)₁₋₇—, —C(═O)—(CH₂)₀₋₃-(alkenylene)-(CH₂)₀₋₃—, —C(═O)—(CH₂)₀₋₃-(alkynylene)-(CH₂)₀₋₃—, —C(═O)—(CH₂)₀₋₃-(3-8 membered carbocyclyl)-(CH₂)₀₋₃—, —C(═O)—(CH₂)₀₋₃-(3-8 membered heterocarbocyclyl)-(CH₂)₀₋₃—, —(CH₂)₀₋₃-(alkenylene)-(CH₂)₀₋₃—, —(CH₂)₀₋₃-(alkynylene)-(CH₂)₀₋₃—, —(CH₂)₀₋₃-(3-8 membered carbocyclyl)-(CH₂)₀₋₃—, or —(CH₂)₀₋₃-(3-8 membered heterocarbocyclyl)-(CH₂)₀₋₃—. In some embodiments, the linker L¹ is —C(═O)—(CH₂)₁₋₈—, —(CH₂)₁₋₉—, —(CH₂)₁₋₂—C(═O)—NH—(CH₂)₂₋₉—, —(CH₂)₁₋₂—C(═O)—NH—(CH₂)₁₋₃—(OCH₂CH₂)₁₋₇—, —(CH₂)₀₋₁—C(═O)—(CH₂)₁₋₃—(OCH₂CH₂)₁₋₇—, —C(═O)—(CH₂)₀₋₃-(3-8 membered carbocyclyl)-(CH₂)₀₋₃—, —C(═O)—(CH₂)₀₋₃-(3-8 membered heterocarbocyclyl)-(CH₂)₀₋₃—, —(CH₂)₀₋₃-(3-8 membered carbocyclyl)-(CH₂)₀₋₃—, or —(CH₂)₀₋₃-(3-8 membered heterocarbocyclyl)-(CH₂)₀₋₃—. In some embodiments, the linker L¹ is —C(═O)—(CH₂)₁₋₈—, —(CH₂)₁₋₉—, —(CH₂)₁₋₂—C(═O)—NH—(CH₂)₂₋₉—, —(CH₂)₁₋₂—C(═O)—NH—(CH₂)₁₋₃—(OCH₂CH₂)₁₋₇—, —(CH₂)₀₋₁—C(═O)—(CH₂)₁₋₃—(OCH₂CH₂)₁₋₇—, —C(═O)—(CH₂)₀₋₃-(3-6 membered carbocyclyl)- (CH₂)₀₋₃—, —C(═O)—(CH₂)₀₋₃-(3-6 membered heterocarbocyclyl)-(CH₂)₀₋₃—, —(CH₂)O₃-(3-8 membered carbocyclyl)-(CH₂)₀₋₃—, or —(CH₂)O₃-(3-6 membered heterocarbocyclyl)-(CH₂)₀₋₃—.

In some embodiments, a linker has the structure —(CH₂)₁₋₁₂—.

In some embodiments, a linker has the structure —(CH₂)₁—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, or —(CH₂)₁₂—.

In some embodiments, a linker has the structure —C(═O)(CH₂)₁₋₁₂—.

In some embodiments, a linker has the structure —C(═O)(CH₂)—, —C(═O)(CH₂)₂—, —C(═O)(CH₂)₃—, —C(═O)(CH₂)₄—, —C(═O)(CH₂)₅—, —C(═O)(CH₂)₆—, —C(═O)(CH₂)₇—, —C(═O)(CH₂)₈—, —C(═O)(CH₂)₉—, C(═O)(CH₂)₁₀—, —C(═O)(CH₂)₁₁—, or —C(═O)(CH₂)₁₂—.

In some embodiments, a linker has the structure —(CH₂)₀₋₁₂NH(CH₂)₁₋₁₂—.

In some embodiments, a linker has the structure —(CH₂)₀₋₂NH(CH₂)₁₋₁₂—.

In some embodiments, a linker has the structure —NH(CH₂)—, —NH(CH₂)₂—, —NH(CH₂)₃—, —NH(CH₂)₄—, —NH(CH₂)₅—, —NH(CH₂)₆—, —NH(CH₂)₇—, —NH(CH₂)₈—, —NH(CH₂)₉—, —NH(CH₂)₁₀—, —NH(CH₂)₁₁—, or —NH(CH₂)₁₂—.

In some embodiments, a linker has the structure —(CH₂)NH(CH₂)—, —(CH₂)NH(CH₂)₂—, —(CH₂)NH(CH₂)₃—, —(CH₂)NH(CH₂)₄—, —(CH₂)NH(CH₂)₅—, —(CH₂)NH(CH₂)₆—, —(CH₂)NH(CH₂)₇—, —(CH₂)NH(CH₂)₈—, —(CH₂)NH(CH₂)₉—, —(CH₂)NH(CH₂)₁₀—, —(CH₂)NH(CH₂)₁₁—, or —(CH₂)NH(CH₂)₁₂—.

In some embodiments, a linker has the structure —(CH₂)₂NH(CH₂)—, —(CH₂)₂NH(CH₂)₂—, —(CH₂)₂NH(CH₂)₃—, —(CH₂)₂NH(CH₂)₄—, —(CH₂)₂NH(CH₂)₅—, —(CH₂)₂NH(CH₂)₆—, —(CH₂)₂NH(CH₂)₇—, —(CH₂)₂NH(CH₂)₈—, —(CH₂)₂NH(CH₂)₉—, —(CH₂)₂NH(CH₂)₁₀—, —(CH₂)₂NH(CH₂)₁₁—, or —(CH₂)₂NH(CH₂)₁₂—.

In some embodiments, a linker has the structure —(CH₂)₀₋₁₂NHC(═O)(CH₂)₁₋₁₂—.

In some embodiments, a linker has the structure —NHC(═O)(CH₂)—, —NHC(═O)(CH₂)₂—, —NHC(═O)(CH₂)₃—, —NHC(═O)(CH₂)₄—, —NHC(═O)(CH₂)₅—, —NHC(═O)(CH₂)₆—, —NHC(═O)(CH₂)₇—, —NHC(═O)(CH₂)₈—, —NHC(═O)(CH₂)₉—, —NHC(═O)(CH₂)₁₀—, —NHC(═O)(CH₂)₁₁—, or —NHC(═O)(CH₂)₁₂—.

In some embodiments, a linker has the structure —(CH₂)NHC(═O)(CH₂)—, —(CH₂) NHC(═O)(CH₂)₂—, —(CH₂)NHC(═O)(CH₂)₃—, —(CH₂)NHC(═O)(CH₂)₄—, —(CH₂)NHC(═O)(CH₂)₅—, —(CH₂)NHC(═O)(CH₂)₆—, —(CH₂)NHC(═O)(CH₂)₇—, —(CH₂)NHC(═O)(CH₂)₈—, —(CH₂)NHC(═O)(CH₂)₉—, —(CH₂)NHC(═O)(CH₂)₁₀—, —(CH₂)NHC(═O)(CH₂)₁₁—, or —(CH₂)NHC(═O)(CH₂)₁₂—.

In some embodiments, a linker has the structure —(CH₂)₂NHC(═O)(CH₂)—, —(CH₂)₂NHC(═O)(CH₂)₂—, —(CH₂)₂NHC(═O)(CH₂)₃—, —(CH₂)₂NHC(═O)(CH₂)₄—, —(CH₂)₂NHC(═O)(CH₂)₅—, —(CH₂)₂NHC(═O)(CH₂)₆—, —(CH₂)₂NHC(═O)(CH₂)₇—, —(CH₂)₂NHC(═O)(CH₂)₈—, —(CH₂)₂NHC(═O)(CH₂)₉—, —(CH₂)₂NHC(═O)(CH₂)₁₀—, —(CH₂)₂NHC(═O)(CH₂)₁₁—, or —(CH₂)₂NHC(═O)(CH₂)₁₂—.

In some embodiments, a linker has the structure —(CH₂)₀₋₁₂C(═O)NH(CH₂)₁₋₁₂—.

In some embodiments, a linker has the structure —(CH₂)₀₋₃C(═O)NH(CH₂)₁₋₁₂—.

In some embodiments, a linker has the structure —C(═O)NH(CH₂)—, —C(═O)NH(CH₂)₂—, —C(═O)NH(CH₂)₃—, —C(═O)NH(CH₂)₄—, —C(═O)NH(CH₂)₅—, —C(═O)NH(CH₂)₆—, —C(═O)NH(CH₂)₇—, —C(═O)NH(CH₂)₈—, —C(═O)NH(CH₂)₉—, —C(═O)NH(CH₂)₁₀—, —C(═O)NH(CH₂)₁₁— or —C(═O)NH(CH₂)₁₂—.

In some embodiments, a linker has the structure —(CH₂)C(═O)NH—(CH₂)—, —(CH₂)C(═O)NH—(CH₂)₂—, —(CH₂)C(═O)NH(CH₂)₃—, —(CH₂)C(═O)NH(CH₂)₄—, —(CH₂)C(═O)NH(CH₂)₅—, —(CH₂)C(═O)NH(CH₂)₆—, —(CH₂)C(═O)NH(CH₂)₇—, —(CH₂)C(═O)NH(CH₂)₈—, —(CH₂)C(═O)NH(CH₂)₉—, —(CH₂)C(═O)NH(CH₂)₁₀—, —(CH₂)C(═O)NH(CH₂)₁₁—, or —(CH₂)C(═O)NH(CH₂)₁₂—.

In some embodiments, a linker has the structure —(CH₂)₂C(═O)NH(CH₂)—, —(CH₂)₂C(═O)NH(CH₂)₂—, —(CH₂)₂C(═O)NH(CH₂)₃—, —(CH₂)₂C(═O)NH(CH₂)₄—, —(CH₂)₂C(═O)NH(CH₂)₅—, —(CH₂)₂C(═O)NH(CH₂)₆—, —(CH₂)₂C(═O)NH(CH₂)₇—, —(CH₂)₂C(═O)NH(CH₂)₈—, —(CH₂)₂C(═O)NH(CH₂)₉—, —(CH₂)₂C(═O)NH(CH₂)₁₀—, —(CH₂)₂C(═O)NH(CH₂)₁₁—, or —(CH₂)₂C(═O)NH(CH₂)₁₂—.

In some embodiments, a linker has the structure —(CH₂)₃C(═O)NH(CH₂)—, —(CH₂)₃C(═O)NH(CH₂)₂—, —(CH₂)₃C(═O)NH(CH₂)₃—, —(CH₂)₃C(═O)NH(CH₂)₄—, —(CH₂)₃C(═O)NH(CH₂)₅—, —(CH₂)₃C(═O)NH(CH₂)₆—, —(CH₂)₃C(═O)NH(CH₂)₇—, —(CH₂)₃C(═O)NH(CH₂)₈—, —(CH₂)₃C(═O)NH(CH₂)₉—, —(CH₂)₃C(═O)NH(CH₂)₁₀—, —(CH₂)₃C(═O)NH(CH₂)₁₁—, or —(CH₂)₃C(═O)NH(CH₂)₁₂—.

In some embodiments, a linker has the structure —(CH₂)₀₋₁₂(CH₂CH₂O)₁₋₁₂(CH₂)₀₋₁₂—.

In some embodiments, a linker has the structure —(CH₂CH₂O)₁₋₁₂(CH₂)₀₋₁₂—.

In some embodiments, a linker has the structure —(CH₂CH₂O)₁₋₁₂(CH₂)₂—.

In some embodiments, a linker has the structure —(CH₂CH₂O)(CH₂)₂—, —(CH₂CH₂O)₂(CH₂)₂—, —(CH₂CH₂O)₃(CH₂)₂—, —(CH₂CH₂O)₄(CH₂)₂—, —(CH₂CH₂O)₅(CH₂)₂—, —(CH₂CH₂O)₆(CH₂)₂—, —(CH₂CH₂O)₇(CH₂)₂—, —(CH₂CH₂O)₈(CH₂)₂—, —(CH₂CH₂O)₉(CH₂)₂—, —(CH₂CH₂O)₁₀(CH₂)₂—, —(CH₂CH₂O)₁₁(CH₂)₂—, or —(CH₂CH₂O)₁₂(CH₂)₂—.

In some embodiments, a linker has the structure —(CH₂)₀₋₁₂C(═O)(CH₂CH₂O)₁₋₁₂(CH₂)₀₋₁₂—.

In some embodiments, a linker has the structure —C(═O)(CH₂CH₂O)₁₋₁₂(CH₂)₀₋₁₂—.

In some embodiments, a linker has the structure —C(═O)(CH₂CH₂O)₁₋₁₂(CH₂)₂—.

In some embodiments, a linker has the structure —C(═O)(CH₂CH₂O)(CH₂)₂—, —C(═O)(CH₂CH₂O)₂(CH₂)₂—, —C(═O)(CH₂CH₂O)₃(CH₂)₂—, —C(═O)(CH₂CH₂O)₄(CH₂)₂—, —C(═O)(CH₂CH₂O)₅(CH₂)₂—, —C(═O)(CH₂CH₂O)₆(CH₂)₂—, —C(═O)(CH₂CH₂O)₇(CH₂)₂—, —C(═O)(CH₂CH₂O)₈(CH₂)₂—, —C(═O)(CH₂CH₂O)₉(CH₂)₂—, —C(═O)(CH₂CH₂O)₁₀(CH₂)₂—, —C(═O)(CH₂CH₂O)₁₁(CH₂)₂—, or —C(═O)(CH₂CH₂O)₁₂(CH₂)₂—.

In some embodiments, a linker has the structure —(CH₂)₀₋₁₂NH(CH₂CH₂O)₁₋₁₂(CH₂)₂—.

In some embodiments, a linker has the structure —(CH₂)₀₋₂NH(CH₂CH₂O)₁₋₁₂(CH₂)₂—.

In some embodiments, a linker has the structure —NH(CH₂CH₂O)(CH₂)₂—, —NH(CH₂CH₂O)₂(CH₂)₂—, —NH(CH₂CH₂O)₃(CH₂)₂—, —NH(CH₂CH₂O)₄(CH₂)₂—, —NH(CH₂CH₂O)₅(CH₂)₂—, —NH(CH₂CH₂O)₆(CH₂)₂—, —NH(CH₂CH₂O)₇(CH₂)₂—, —NH(CH₂CH₂O)₈(CH₂)₂—, —NH(CH₂CH₂O)₉(CH₂)₂—, —NH(CH₂CH₂O)₁₀(CH₂)₂—, —NH(CH₂CH₂O)₁₁(CH₂)₂—, or —NH(CH₂CH₂O)₁₂(CH₂)₂—.

In some embodiments, a linker has the structure —(CH₂)NH(CH₂CH₂O)(CH₂)₂—, —(CH₂)NH(CH₂CH₂O)₂(CH₂)₂—, —(CH₂)NH(CH₂CH₂O)₃(CH₂)₂—, —(CH₂)NH(CH₂CH₂O)₄(CH₂)₂—, —(CH₂)NH(CH₂CH₂O)₅(CH₂)₂—, —(CH₂)NH(CH₂CH₂O)₆(CH₂)₂—, —(CH₂)NH(CH₂CH₂O)₇(CH₂)₂—, —(CH₂)NH(CH₂CH₂O)₈(CH₂)₂—, —(CH₂)NH(CH₂CH₂O)₉(CH₂)₂—, —(CH₂)NH(CH₂CH₂O)₁₀(CH₂)₂—, —(CH₂)NH(CH₂CH₂O)₁₁(CH₂)₂—, or —(CH₂)NH(CH₂CH₂O)₁₂(CH₂)₂—.

In some embodiments, a linker has the structure —(CH₂)₂NH(CH₂CH₂O)(CH₂)₂—, —(CH₂)₂NH(CH₂CH₂O)₂(CH₂)₂—, —(CH₂)₂NH(CH₂CH₂O)₃(CH₂)₂—, —(CH₂)₂NH(CH₂CH₂O)₄(CH₂)₂—, —(CH₂)₂NH(CH₂CH₂O)₅(CH₂)₂—, —(CH₂)₂NH(CH₂CH₂O)₆(CH₂)₂—, —(CH₂)₂NH(CH₂CH₂O)₇(CH₂)₂—, —(CH₂)₂NH(CH₂CH₂O)₈(CH₂)₂—, —(CH₂)₂NH(CH₂CH₂O)₉(CH₂)₂—, —(CH₂)₂NH(CH₂CH₂O)₁₀(CH₂)₂—, —(CH₂)₂NH(CH₂CH₂O)₁₁(CH₂)₂—, or —(CH₂)₂NH(CH₂CH₂O)₁₂(CH₂)₂—.

In some embodiments, a linker has the structure —(CH₂)₀₋₁₂NHC(═O)(CH₂CH₂O)₁₋₁₂(CH₂)₂—.

In some embodiments, a linker has the structure —NHC(═O)(CH₂CH₂O)(CH₂)₂—, —NHC(═O)(CH₂CH₂O)₂(CH₂)₂—, —NHC(═O)(CH₂CH₂O)₃(CH₂)₂—, —NHC(═O)(CH₂CH₂O)₄(CH₂)₂—, —NHC(═O)(CH₂CH₂O)₅(CH₂)₂—, —NHC(═O)(CH₂CH₂O)₆(CH₂)₂—, —NHC(═O)(CH₂CH₂O)₇(CH₂)₂—, —NHC(═O)(CH₂CH₂O)₈(CH₂)₂—, —NHC(═O)(CH₂CH₂O)₉(CH₂)₂—, —NHC(═O)(CH₂CH₂O)₁₀(CH₂)₂—, —NHC(═O)(CH₂CH₂O)₁₁(CH₂)₂—, or —NHC(═O)(CH₂CH₂O)₁₂(CH₂)₂—.

In some embodiments, a linker has the structure —(CH₂)NHC(═O)(CH₂CH₂O)(CH₂)₂—, —(CH₂)NHC(═O)(CH₂CH₂O)₂(CH₂)₂—, —(CH₂)NHC(═O)(CH₂CH₂O)₃(CH₂)₂—, —(CH₂)NHC(═O)(CH₂CH₂O)₄(CH₂)₂—, —(CH₂)NHC(═O)(CH₂CH₂O)₅(CH₂)₂—, —(CH₂)NHC(═O)(CH₂CH₂O)₆(CH₂)₂—, —(CH₂)NHC(═O)(CH₂CH₂O)₇(CH₂)₂—, —(CH₂)NHC(═O)(CH₂CH₂O)₈(CH₂)₂—, —(CH₂)NHC(═O)(CH₂CH₂O)₉(CH₂)₂—, —(CH₂)NHC(═O)(CH₂CH₂O)₁₀(CH₂)₂—, —(CH₂)NHC(═O)(CH₂CH₂O)₁₁(CH₂)₂—, or —(CH₂)NHC(═O)(CH₂CH₂O)₁₂(CH₂)₂—.

In some embodiments, a linker has the structure —(CH₂)₂NHC(═O)(CH₂CH₂O)(CH₂)₂—, —(CH₂)₂NHC(═O)(CH₂CH₂O)₂(CH₂)₂—, —(CH₂)₂NHC(═O)(CH₂CH₂O)₃(CH₂)₂—, —(CH₂)₂NHC(═O)(CH₂CH₂O)₄(CH₂)₂—, —(CH₂)₂NHC(═O)(CH₂CH₂O)₅(CH₂)₂—, —(CH₂)₂NHC(═O)(CH₂CH₂O)₆(CH₂)₂—, —(CH₂)₂NHC(═O)(CH₂CH₂O)₇(CH₂)₂—, —(CH₂)₂NHC(═O)(CH₂CH₂O)₈(CH₂)₂—, —(CH₂)₂NHC(═O)(CH₂CH₂O)₉(CH₂)₂—, —(CH₂)₂NHC(═O)(CH₂CH₂O)₁₀(CH₂)₂—, —(CH₂)₂NHC(═O)(CH₂CH₂O)₁₁(CH₂)₂—, or —(CH₂)₂NHC(═O)(CH₂CH₂O)₁₂(CH₂)₂—.

In some embodiments, a linker has the structure —(CH₂)₀₋₁₂C(═O)NH(CH₂CH₂O)₁₋₁₂(CH₂)₂—.

In some embodiments, a linker has the structure —(CH₂)₀₋₂C(═O)NH(CH₂CH₂O)₁₋₁₂(CH₂)₂—.

In some embodiments, a linker has the structure —C(═O)NH(CH₂CH₂O)(CH₂)₂—, —C(═O)NH(CH₂CH₂O)₂(CH₂)₂—, —C(═O)NH(CH₂CH₂O)₃(CH₂)₂—, —C(═O)NH(CH₂CH₂O)₄(CH₂)₂—, —C(═O)NH(CH₂CH₂O)₅(CH₂)₂—, —C(═O)NH(CH₂CH₂O)₆(CH₂)₂—, —C(═O)NH(CH₂CH₂O)₇(CH₂)₂—, —C(═O)NH(CH₂CH₂O)₈(CH₂)₂—, —C(═O)NH(CH₂CH₂O)₉(CH₂)₂—, —C(═O)NH(CH₂CH₂O)₁₀(CH₂)₂—, —C(═O)NH(CH₂CH₂O)₁₁(CH₂)₂—, or —C(═O)NH(CH₂CH₂O)₁₂(CH₂)₂—.

In some embodiments, a linker has the structure —(CH₂)C(═O)NH(CH₂CH₂O)(CH₂)₂—, —(CH₂)C(═O)NH(CH₂CH₂O)₂(CH₂)₂—, —(CH₂)C(═O)NH(CH₂CH₂O)₃(CH₂)₂—, —(CH₂)C(═O)NH(CH₂CH₂O)₄(CH₂)₂—, —(CH₂)C(═O)NH(CH₂CH₂O)₅(CH₂)₂—, —(CH₂)C(═O)NH(CH₂CH₂O)₆(CH₂)₂—, —(CH₂)C(═O)NH(CH₂CH₂O)₇(CH₂)₂—, —(CH₂)C(═O)NH(CH₂CH₂O)₈(CH₂)₂—, —(CH₂)C(═O)NH(CH₂CH₂O)₉(CH₂)₂—, —(CH₂)C(═O)NH(CH₂CH₂O)₁₀(CH₂)₂—, —(CH₂)C(═O)NH(CH₂CH₂O)₁₁(CH₂)₂—, or —(CH₂)C(═O)NH(CH₂CH₂O)₁₂(CH₂)₂—.

In some embodiments, a linker has the structure —(CH₂)₂C(═O)NH(CH₂CH₂O)(CH₂)₂—, —(CH₂)₂C(═O)NH(CH₂CH₂O)₂(CH₂)₂—, —(CH₂)₂C(═O)NH(CH₂CH₂O)₃(CH₂)₂—, —(CH₂)₂C(═O)NH(CH₂CH₂O)₄(CH₂)₂—, —(CH₂)₂C(═O)NH(CH₂CH₂O)₅(CH₂)₂—, —(CH₂)₂C(═O)NH(CH₂CH₂O)₆(CH₂)₂—, —(CH₂)₂C(═O)NH(CH₂CH₂O)₇(CH₂)₂—, —(CH₂)₂C(═O)NH(CH₂CH₂O)₈(CH₂)₂—, —(CH₂)₂C(═O)NH(CH₂CH₂O)₉(CH₂)₂—, —(CH₂)₂C(═O)NH(CH₂CH₂O)₁₀(CH₂)₂—, —(CH₂)₂C(═O)NH(CH₂CH₂O)₁₁(CH₂)₂—, or —(CH₂)₂C(═O)NH(CH₂CH₂O)₁₂(CH₂)₂—.

In some embodiments, a linker has the structure —(CH₂)₃C(═O)NH(CH₂CH₂O)(CH₂)₂—, —(CH₂)₃C(═O)NH(CH₂CH₂O)₂(CH₂)₂—, —(CH₂)₃C(═O)NH(CH₂CH₂O)₃(CH₂)₂—, —(CH₂)₃C(═O)NH(CH₂CH₂O)₄(CH₂)₂—, —(CH₂)₃C(═O)NH(CH₂CH₂O)₅(CH₂)₂—, —(CH₂)₃C(═O)NH(CH₂CH₂O)₆(CH₂)₂—, —(CH₂)₃C(═O)NH(CH₂CH₂O)₇(CH₂)₂—, —(CH₂)₃C(═O)NH(CH₂CH₂O)₈(CH₂)₂—, —(CH₂)₃C(═O)NH(CH₂CH₂O)₉(CH₂)₂—, —(CH₂)₃C(═O)NH(CH₂CH₂O)₁₀(CH₂)₂—, —(CH₂)₃C(═O)NH(CH₂CH₂O)₁₁(CH₂)₂—, or —(CH₂)₃C(═O)NH(CH₂CH₂O)₁₂(CH₂)₂—.

In some embodiments, the linker L¹ has the structure —(CH₂)₀₋₁₂NH(CH₂)₂₋₁₂NH—. In some embodiments, the linker has the structure —NH(CH₂)₂NH—, —NH(CH₂)₃NH—, —NH(CH₂)₄NH—, —NH(CH₂)₅NH—, —NH(CH₂)₆NH—, —NH(CH₂)₇NH—, —NH(CH₂)₈NH—, —NH(CH₂)₉NH—, —NH(CH₂)₁₀NH—, —NH(CH₂)₁₁NH—, or —NH(CH₂)₁₂NH—. In some embodiments, the linker has the structure —(CH₂)₀₋₁₂NHC(═O)(CH₂)₂₋₁₂NH—. In some embodiments, the linker has the structure —NHC(═O)(CH₂)₂NH—, —NHC(═O)(CH₂)₃NH—, —NHC(═O)(CH₂)₄NH—, —NHC(═O)(CH₂)₅NH—, —NHC(═O)(CH₂)₆NH—, —NHC(═O)(CH₂)₇NH—, —NHC(═O)(CH₂)₈NH—, —NHC(═O)(CH₂)₉NH—, —NHC(═O)(CH₂)₁₀NH—, —NHC(═O)(CH₂)₁₁NH—, or —NHC(═O)(CH₂)₁₂NH—. In some embodiments, the linker has the structure —(CH₂)₀₋₁₂NH(CH₂)₂₋₁₂C(═O)NH—. In some embodiments, the linker has the structure —NH(CH₂)₂C(═O)NH—, —NH(CH₂)₃C(═O)NH—, —NH(CH₂)₄C(═O)NH—, —NH(CH₂)₅C(═O)NH—, —NH(CH₂)₆C(═O)NH—, —NH(CH₂)₇C(═O)NH—, —NH(CH₂)₈C(═O)NH—, —NH(CH₂)₉C(═O)NH—, —NH(CH₂)₁₀C(═O)NH—, —NH(CH₂)₁₁C(═O)NH—, or —NH(CH₂)₁₂(═O)NH—. In some embodiments, the linker has the structure —(CH₂)₀₋₁₂C(═O)NH(CH₂)₂₋₁₂C(═O)NH—, In some embodiments, the linker has the structure —C(═O)NH(CH₂)₂C(═O)NH—, —C(═O)NH(CH₂)₃C(═O)NH—, —C(═O)NH(CH₂)₄C(═O)NH—, —C(═O)NH(CH₂)₅C(═O)NH—, —C(═O)NH(CH₂)₆C(═O)NH—, —C(═O)NH(CH₂)₇C(═O)NH—, —C(═O)NH(CH₂)₈C(═O)NH—, —C(═O)NH(CH₂)₉C(═O)NH—, —C(═O)NH(CH₂)₁₀C(═O)NH—, —C(═O)NH(CH₂)₁₁C(═O)NH—, or —C(═O)NH(CH₂)₁₂(═O)NH—. In some embodiments, the linker has the structure —(CH₂)C(═O)NH(CH₂)₂C(═O)NH—, —(CH₂)C(═O)NH(CH₂)₃C(═O)NH—, —(CH₂)C(═O)NH(CH₂)₄C(═O)NH—, —(CH₂)C(═O)NH(CH₂)₅C(═O)NH—, —(CH₂)C(═O)NH(CH₂)₆C(═O)NH—, —(CH₂)C(═O)NH(CH₂)₇C(═O)NH—, —(CH₂)C(═O)NH(CH₂)₈C(═O)NH—, —(CH₂)C(═O)NH(CH₂)₉C(═O)NH—, —(CH₂)C(═O)NH(CH₂)₁₀C(═O)NH—, —(CH₂)C(═O)NH(CH₂)₁₁C(═O)NH—, or —(CH₂)C(═O)NH(CH₂)₁₂(═O)NH—. In some embodiments, the linker has the structure —(CH₂)₂C(═O)NH(CH₂)₂C(═O)NH—, —(CH₂)₂C(═O)NH(CH₂)₃C(═O)NH—, —(CH₂)₂C(═O)NH(CH₂)₄C(═O)NH—, —(CH₂)₂C(═O)NH(CH₂)₅C(═O)NH—, —(CH₂)₂C(═O)NH(CH₂)₆C(═O)NH—, —(CH₂)₂C(═O)NH(CH₂)₇C(═O)NH—, —(CH₂)₂C(═O)NH(CH₂)₈C(═O)NH—, —(CH₂)₂C(═O)NH(CH₂)₉C(═O)NH—, —(CH₂)₂C(═O)NH(CH₂)₁₀C(═O)NH—, —(CH₂)₂C(═O)NH(CH₂)₁₁C(═O)NH—, or —(CH₂)₂C(═O)NH(CH₂)₁₂(═O)NH—. In some embodiments, the linker has the structure —(CH₂)₃C(═O)NH(CH₂)₂C(═O)NH—, —(CH₂)₃C(═O)NH(CH₂)₃C(═O)NH—, —(CH₂)₃C(═O)NH(CH₂)₄C(═O)NH—, —(CH₂)₃C(═O)NH(CH₂)₅C(═O)NH—, —(CH₂)₃C(═O)NH(CH₂)₆C(═O)NH—, —(CH₂)₃C(═O)NH(CH₂)₇C(═O)NH—, —(CH₂)₃C(═O)NH(CH₂)₈C(═O)NH—, —(CH₂)₃C(═O)NH(CH₂)₉C(═O)NH—, —(CH₂)₃C(═O)NH(CH₂)₁₀C(═O)NH—, —(CH₂)₃C(═O)NH(CH₂)₁₁C(═O)NH—, or —(CH₂)₃C(═O)NH(CH₂)₁₂(═O)NH—.

In some embodiments, the linker L¹ has the structure —(CH₂)₀₋₁₂NH(CH₂CH₂O)₁₋₁₂(CH₂)₂NH—. In some embodiments, the linker has the structure —NH(CH₂CH₂O)(CH₂)₂NH—, —NH(CH₂CH₂O)₂(CH₂)₂NH—, —NH(CH₂CH₂O)₃(CH₂)₂NH—, —NH(CH₂CH₂O)₄(CH₂)₂NH—, —NH(CH₂CH₂O)₅(CH₂)₂NH—, —NH(CH₂CH₂O)₆(CH₂)₂NH—, —NH(CH₂CH₂O)₇(CH₂)₂NH—, —NH(CH₂CH₂O)₈(CH₂)₂NH—, —NH(CH₂CH₂O)₉(CH₂)₂NH—, —NH(CH₂CH₂O)₁₀(CH₂)₂NH—, —NH(CH₂CH₂O)₁₁(CH₂)₂NH—, or —NH(CH₂CH₂O)₁₂(CH₂)₂NH—. In some embodiments, the linker has the structure —(CH₂)₀₋₁₂NHC(═O)(CH₂CH₂O)₁₋₁₂(CH₂)₂NH—. In some embodiments, the linker has the structure —(CH₂)₀₋₁₂NH(CH₂CH₂O)₁₋₁₂(CH₂)₂C(═O)NH—. In some embodiments, the linker has the structure —NH(CH₂CH₂O)(CH₂)₂C(═O)NH—, —NH(CH₂CH₂O)₂(CH₂)₂C(═O)NH—, —NH(CH₂CH₂O)₃(CH₂)₂C(═O)NH—, —NH(CH₂CH₂O)₄(CH₂)₂C(═O)NH—, —NH(CH₂CH₂O)₅(CH₂)₂C(═O)NH—, —NH(CH₂CH₂O)₆(CH₂)₂C(═O)NH—, —NH(CH₂CH₂O)₇(CH₂)₂C(═O)NH—, —NH(CH₂CH₂O)₈(CH₂)₂C(═O)NH—, —NH(CH₂CH₂O)₉(CH₂)₂C(═O)NH—, —NH(CH₂CH₂O)₁₀(CH₂)₂C(═O)NH—, —NH(CH₂CH₂O)₁₁(CH₂)₂C(═O)NH—, or —NH(CH₂CH₂O)₁₂(CH₂)₂C(═O)NH—. In some embodiments, the linker has the structure —(CH₂)₀₋₁₂C(═O)NH(CH₂CH₂O)₁₋₁₂(CH₂)₂C(═O)NH—. In some embodiments, the linker has the structure —C(═O)NH(CH₂CH₂O)(CH₂)₂C(═O)NH—, —C(═O)NH(CH₂CH₂O)₂(CH₂)₂C(═O)NH—, —C(═O)NH(CH₂CH₂O)₃(CH₂)₂C(═O)NH—, —C(═O)NH(CH₂CH₂O)₄(CH₂)₂C(═O)NH—, —C(═O)NH(CH₂CH₂O)₅(CH₂)₂C(═O)NH—, —C(═O)NH(CH₂CH₂O)₆(CH₂)₂C(═O)NH—, —C(═O)NH(CH₂CH₂O)₇(CH₂)₂C(═O)NH—, —C(═O)NH(CH₂CH₂O)₈(CH₂)₂C(═O)NH—, —C(═O)NH(CH₂CH₂O)₉(CH₂)₂C(═O)NH—, —C(═O)NH(CH₂CH₂O)₁₀(CH₂)₂C(═O)NH—, —C(═O)NH(CH₂CH₂O)₁₁(CH₂)₂C(═O)NH—, or —C(═O)NH(CH₂CH₂O)₁₂(CH₂)₂C(═O)NH—. In some embodiments, the linker has the structure —(CH₂)C(═O)NH(CH₂CH₂O)(CH₂)₂C(═O)NH—, —(CH₂)C(═O)NH(CH₂CH₂O)₂(CH₂)₂C(═O)NH—, —(CH₂)C(═O)NH(CH₂CH₂O)₃(CH₂)₂C(═O)NH—, —(CH₂)C(═O)NH(CH₂CH₂O)₄(CH₂)₂C(═O)NH—, —(CH₂)C(═O)NH(CH₂CH₂O)₅(CH₂)₂C(═O)NH—, —(CH₂)C(═O)NH(CH₂CH₂O)₆(CH₂)₂C(═O)NH—, —(CH₂)C(═O)NH(CH₂CH₂O)₇(CH₂)₂C(═O)NH—, —(CH₂)C(═O)NH(CH₂CH₂O)₈(CH₂)₂C(═O)NH—, —(CH₂)C(═O)NH(CH₂CH₂O)₉(CH₂)₂C(═O)NH—, —(CH₂)C(═O)NH(CH₂CH₂O)₁₀(CH₂)₂C(═O)NH—, —(CH₂)C(═O)NH(CH₂CH₂O)₁₁(CH₂)₂C(═O)NH—, or —(CH₂)C(═O)NH(CH₂CH₂O)₁₂(CH₂)₂C(═O)NH—. In some embodiments, the linker has the structure —(CH₂)₂C(═O)NH(CH₂CH₂O)(CH₂)₂C(═O)NH—, —(CH₂)₂C(═O)NH(CH₂CH₂O)₂(CH₂)₂C(═O)NH—, —(CH₂)₂C(═O)NH(CH₂CH₂O)₃(CH₂)₂C(═O)NH—, —(CH₂)₂C(═O)NH(CH₂CH₂O)₄(CH₂)₂C(═O)NH—, —(CH₂)₂C(═O)NH(CH₂CH₂O)₅(CH₂)₂C(═O)NH—, —(CH₂)₂C(═O)NH(CH₂CH₂O)₆(CH₂)₂C(═O)NH—, —(CH₂)₂C(═O)NH(CH₂CH₂O)₇(CH₂)₂C(═O)NH—, —(CH₂)₂C(═O)NH(CH₂CH₂O)₈(CH₂)₂C(═O)NH—, —(CH₂)₂C(═O)NH(CH₂CH₂O)₉(CH₂)₂C(═O)NH—, —(CH₂)₂C(═O)NH(CH₂CH₂O)₁₀(CH₂)₂C(═O)NH—, —(CH₂)₂C(═O)NH(CH₂CH₂O)₁₁(CH₂)₂C(═O)NH—, or —(CH₂)₂C(═O)NH(CH₂CH₂O)₁₂(CH₂)₂C(═O)NH—. In some embodiments, the linker has the structure —(CH₂)₃C(═O)NH(CH₂CH₂O)(CH₂)₂C(═O)NH—, —(CH₂)₃C(═O)NH(CH₂CH₂O)₂(CH₂)₂C(═O)NH—, —(CH₂)₃C(═O)NH(CH₂CH₂O)₃(CH₂)₂C(═O)NH—, —(CH₂)₃C(═O)NH(CH₂CH₂O)₄(CH₂)₂C(═O)NH—, —(CH₂)₃C(═O)NH(CH₂CH₂O)₅(CH₂)₂C(═O)NH—, —(CH₂)₃C(═O)NH(CH₂CH₂O)₆(CH₂)₂C(═O)NH—, —(CH₂)₃C(═O)NH(CH₂CH₂O)₇(CH₂)₂C(═O)NH—, —(CH₂)₃C(═O)NH(CH₂CH₂O)₈(CH₂)₂C(═O)NH—, —(CH₂)₃C(═O)NH(CH₂CH₂O)₉(CH₂)₂C(═O)NH—, —(CH₂)₃C(═O)NH(CH₂CH₂O)₁₀(CH₂)₂C(═O)NH—, —(CH₂)₃C(═O)NH(CH₂CH₂O)₁₁(CH₂)₂C(═O)NH—, or —(CH₂)₃C(═O)NH(CH₂CH₂O)₁₂(CH₂)₂C(═O)NH—.

In some embodiments, representative DDB1 binding moieties with a linker component are described in Table 2.

TABLE 2
Representative compound fragments comprising a DDB1 binding moiety and a linker

| Cpd. | | |
|---|---|---|
| No. | Structure | Chemical Name |
| BL1- 1 | 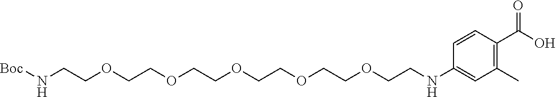 [SMILES: Cc1cc(NCCOCCOCCOCCOCCOCCNC(=O)OC(C)(C)C)ccc1C(=O)O] | 4-((2,2-dimethyl-4-oxo- 3,8,11,14,17,20-hexaoxa-5- azadocosan-22-yl)amino)-2- methylbenzoic acid |
| BL1- 2 | 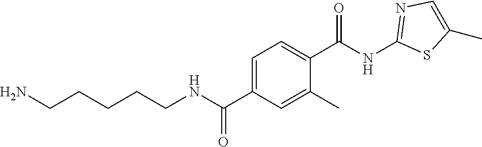 [SMILES: Cc1cnc(NC(=O)c2ccc(C(=O)NCCCCCN)cc2C)s1] | N⁴-(5-aminopentyl)-2-methyl- N¹-(5-methylthiazol-2- yl)terephthalamide |
| BL1- 3 | 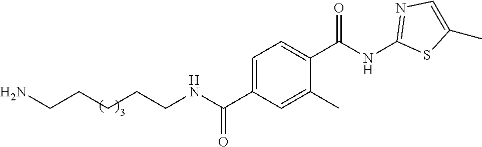 | N⁴-(7-aminoheptyl)-2-methyl- N¹-(5-methylthiazol-2- yl)terephthalamide |
| BL1- 4 | 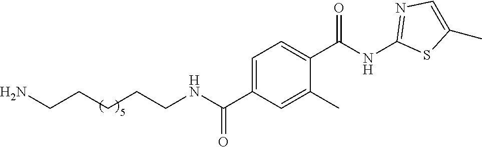 | N⁴-(9-aminononyl)-2-methyl- N¹-(5-methylthiazol-2- yl)terephthalamide |
| BL1- 5 | 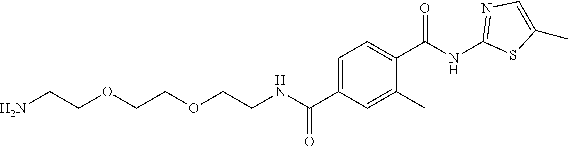 [SMILES: Cc1cnc(NC(=O)c2ccc(C(=O)NCCOCCOCCN)cc2C)s1] | N⁴-(2-(2-(2- aminoethoxy)ethoxy)ethyl)-2- methyl-N¹-(5-methylthiazol-2- yl)terephthalamide |
| BL1- 6 | 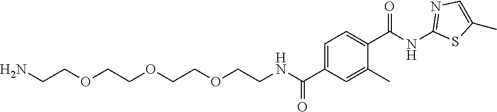 [SMILES: Cc1cnc(NC(=O)c2ccc(C(=O)NCCOCCOCCOCCN)cc2C)s1] | N⁴-(2-(2-(2-(2- aminoethoxy)ethoxy)ethoxy) ethyl)-2-methyl-N¹-(5- methylthiazol-2- yl)terephthalamide |
| BL1- 7 | 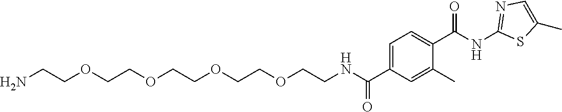 [SMILES: Cc1cnc(NC(=O)c2ccc(C(=O)NCCOCCOCCOCCOCCN)cc2C)s1] | N⁴-(14-amino-3,6,9,12- tetraoxatetradecyl)-2-methyl- N¹-(5-methylthiazol-2- yl)terephthalamide |
| BL1- 8 | 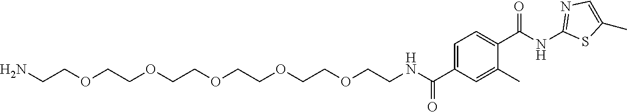 [SMILES: Cc1cnc(NC(=O)c2ccc(C(=O)NCCOCCOCCOCCOCCOCCN)cc2C)s1] | N⁴-(17-amino-3,6,9,12,15- pentaoxaheptadecyl)- 2-methyl- N¹-(5-methylthiazol-2- yl)terephthalamide |
| BL1- 9 | 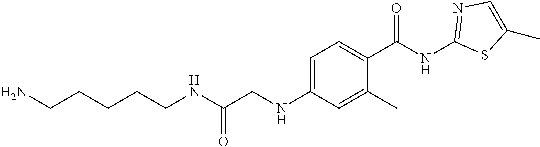 [SMILES: Cc1cnc(NC(=O)c2ccc(NCC(=O)NCCCCCN)cc2C)s1] | 4-((2-((5-aminopentyl) amino)-2-oxoethyl) amino)-2-methyl- N-(5-methylthiazol-2- yl)benzamide |
| BL1- 10 | 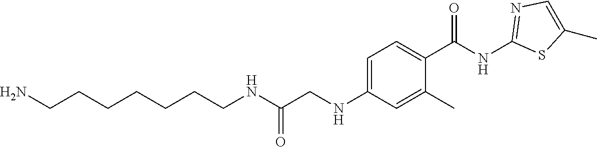 [SMILES: Cc1cnc(NC(=O)c2ccc(NCC(=O)NCCCCCCCN)cc2C)s1] | 4-((2-((7-aminoheptyl) amino)-2-oxoethyl) amino)-2-methyl- N-(5-methylthiazol-2- yl)benzamide |
| BL1- 11 | 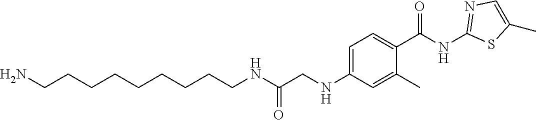 [SMILES: Cc1cnc(NC(=O)c2ccc(NCC(=O)NCCCCCCCCCN)cc2C)s1] | 4-((2-((9-aminononyl)amino)- 2-oxoethyl)amino)-2-methyl- N-(5-methylthiazol-2- yl)benzamide |
| BL1- 12 | 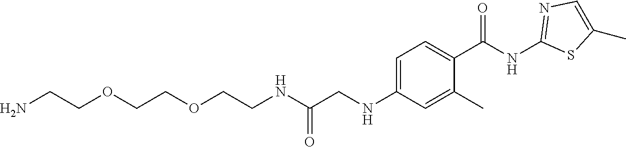 [SMILES: Cc1cnc(NC(=O)c2ccc(NCC(=O)NCCOCCOCCN)cc2C)s1] | 4-((2-((2-(2-(2- aminoethoxy)ethoxy)ethyl) amino)-2-oxoethyl)amino)-2- methyl-N-(5-methylthiazol-2- yl)benzamide |
| BL1- 13 | 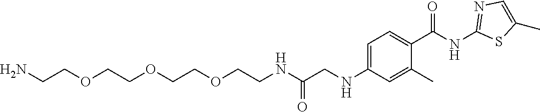 [SMILES: Cc1cnc(NC(=O)c2ccc(NCC(=O)NCCOCCOCCOCCN)cc2C)s1] | 4-((14-amino-2-oxo-6,9,12- trioxa-3-azatetradecyl)amino)- 2-methyl-N-(5-methylthiazol- 2-yl)benzamide |
| BL1- 14 | 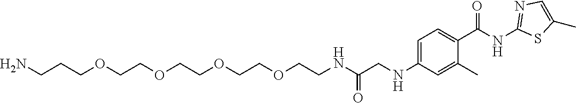 [SMILES: Cc1cnc(NC(=O)c2ccc(NCC(=O)NCCOCCOCCOCCOCCCN)cc2C)s1] | 4-((18-amino-2-oxo- 6,9,12,15-tetraoxa-3- azaoctadecyl)amino)- 2-methyl- N-(5-methylthiazol-2- yl)benzamide |
| BL1- 15 | 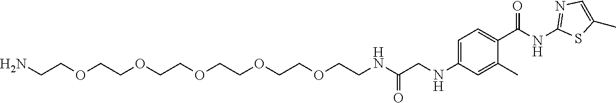 [SMILES: Cc1cnc(NC(=O)c2ccc(NCC(=O)NCCOCCOCCOCCOCCOCCN)cc2C)s1] | 4-((20-amino-2-oxo- 6,9,12,15,18-pentaoxa-3- azaicosyl)amino)-2-methyl- N-(5-methylthiazol-2- yl)benzamide |
| BL1- 16 | 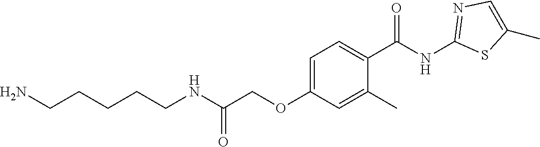 [SMILES: Cc1cnc(NC(=O)c2ccc(OCC(=O)NCCCCCN)cc2C)s1] | 4-(2-((5-aminopentyl)amino)- 2-oxoethoxy)-2-methyl-N-(5- methylthiazol-2-yl)benzamide |
| BL1- 17 | 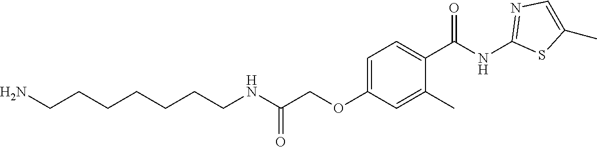 [SMILES: Cc1cnc(NC(=O)c2ccc(OCC(=O)NCCCCCCCN)cc2C)s1] | 4-(2-((7-aminoheptyl)amino)- 2-oxoethoxy)-2-methyl-N-(5- methylthiazol-2-yl)benzamide |
| BL1- 18 | 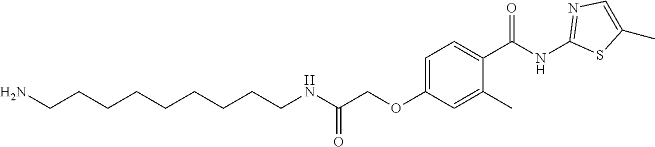 [SMILES: Cc1cnc(NC(=O)c2ccc(OCC(=O)NCCCCCCCCCN)cc2C)s1] | 4-(2-((9-aminononyl) amino)-2-oxoethoxy)- 2-methyl-N-(5- methylthiazol-2-yl)benzamide |
| BL1- 19 | 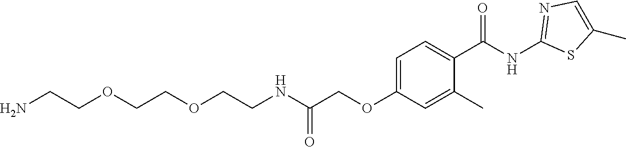 [SMILES: Cc1cnc(NC(=O)c2ccc(OCC(=O)NCCOCCOCCN)cc2C)s1] | 4-(2-((2-(2-(2- aminoethoxy)ethoxy)ethyl) amino)-2-oxoethoxy)-2- methyl-N-(5-methylthiazol-2- yl)benzamide |
| BL1- 20 | 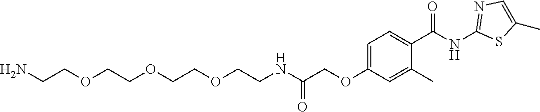 [SMILES: Cc1cnc(NC(=O)c2ccc(OCC(=O)NCCOCCOCCOCCN)cc2C)s1] | 4-((14-amino-2-oxo-6,9,12- trioxa-3-azatetradecyl)oxy)-2- methyl-N-(5-methylthiazol-2- yl)benzamide |
| BL1- 21 | 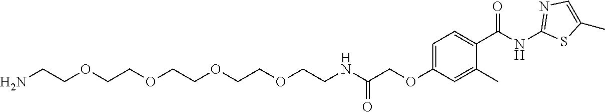 [SMILES: Cc1cnc(NC(=O)c2ccc(OCC(=O)NCCOCCOCCOCCOCCN)cc2C)s1] | 4-((17-amino-2-oxo- 6,9,12,15-tetraoxa- 3-azaheptadecyl)oxy)- 2-methyl-N-(5- methylthiazol- 2-yl)benzamide |
| BL1- 22 | 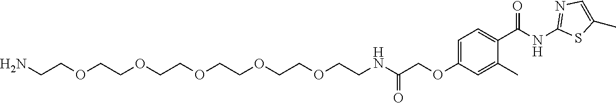 [SMILES: Cc1cnc(NC(=O)c2ccc(OCC(=O)NCCOCCOCCOCCOCCOCCN)cc2C)s1] | 4-((20-amino-2-oxo- 6,9,12,15,18-pentaoxa-3- azaicosyl)oxy)-2-methyl- N-(5-methylthiazol- 2-yl)benzamide |
| BL1- 23 | 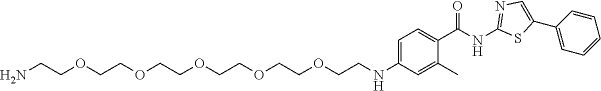 [SMILES: Cc1cc(NCCOCCOCCOCCOCCOCCN)ccc1C(=O)Nc1ncc(-c2ccccc2)s1] | 4-((17-amino-3,6,9,12,15- pentaoxaheptadecyl) amino)-2-methyl-N- (5-phenylthiazol-2- yl)benzamide |
| BL1- 24 | 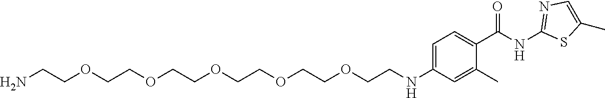 [SMILES: Cc1cnc(NC(=O)c2ccc(NCCOCCOCCOCCOCCOCCN)cc2C)s1] | 4-((17-amino-3,6,9,12,15- pentaoxaheptadecyl) amino)-2-methyl-N- (5-methylthiazol-2- yl)benzamide |
| BL1- 25 | 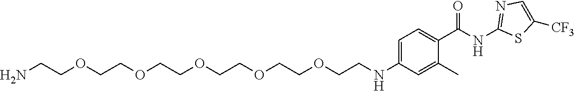 [SMILES: Cc1cc(NCCOCCOCCOCCOCCOCCN)ccc1C(=O)Nc1ncc(C(F)(F)F)s1] | 4-((17-amino-3,6,9,12,15- pentaoxaheptadecyl)amino)- 2-methyl-N-(5- (trifluoromethyl)thiazol-2- yl)benzamide |
| BL1- 26 | 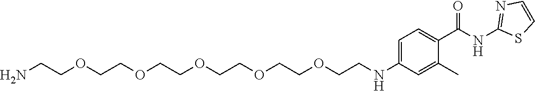 [SMILES: Cc1cc(NCCOCCOCCOCCOCCOCCN)ccc1C(=O)Nc1nccs1] | 4-((17-amino-3,6,9,12,15- pentaoxaheptadecyl)amino)- 2-methyl-N-(thiazol-2- yl)benzamide |
| BL1- 27 | 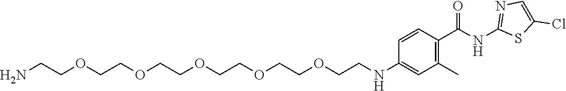 [SMILES: Cc1cc(NCCOCCOCCOCCOCCOCCN)ccc1C(=O)Nc1ncc(Cl)s1] | 4-((17-amino-3,6,9,12,15- pentaoxaheptadecyl)amino)- N-(5-chlorothiazol-2-yl)-2- methylbenzamide |
| BL1- 28 | 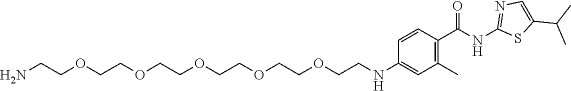 [SMILES: Cc1cc(NCCOCCOCCOCCOCCOCCN)ccc1C(=O)Nc1ncc(C(C)C)s1] | 4-((17-amino-3,6,9,12,15- pentaoxaheptadecyl) amino)-N-(5- isopropylthiazol-2-yl)-2- methylbenzamide |
| BL1- 29 | 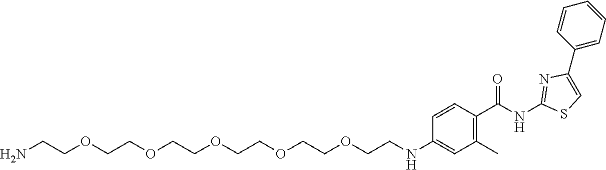 [SMILES: Cc1cc(NCCOCCOCCOCCOCCOCCN)ccc1C(=O)Nc1nc(-c2ccccc2)cs1] | 4-((17-amino-3,6,9,12,15- pentaoxaheptadecyl) amino)-2-methyl-N- (4-phenylthiazol-2- yl)benzamide |
| BL1- 30 | 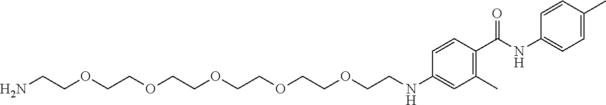 [SMILES: Cc1ccc(NC(=O)c2ccc(NCCOCCOCCOCCOCCOCCN)cc2C)cc1] | 4-((17-amino-3,6,9,12,15- pentaoxaheptadecyl) amino)-2-methyl-N- (p-tolyl)benzamide |
| BL1- 31 | 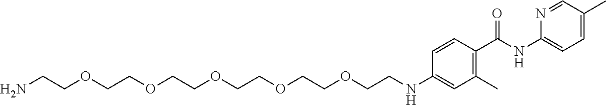 [SMILES: Cc1ccc(NC(=O)c2ccc(NCCOCCOCCOCCOCCOCCN)cc2C)nc1] | 4-((17-amino-3,6,9,12,15- pentaoxaheptadecyl) amino)-2-methyl-N- (5-methylpyridin-2- yl)benzamide |
| BL1- 32 | 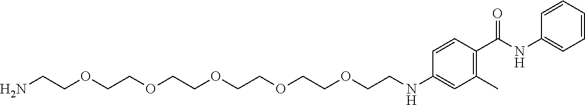 [SMILES: Cc1cc(NCCOCCOCCOCCOCCOCCN)ccc1C(=O)Nc1ccccc1] | 4-((17-amino-3,6,9,12,15- pentaoxaheptadecyl) amino)-2-methyl-N- phenylbenzamide |
| BL1- 33 | 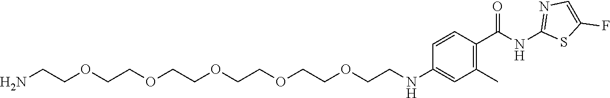 [SMILES: Cc1cc(NCCOCCOCCOCCOCCOCCN)ccc1C(=O)Nc1ncc(F)s1] | 4-((17-amino-3,6,9,12,15- pentaoxaheptadecyl) amino)-N-(5- fluorothiazol-2-yl)-2- methylbenzamide |
| BL1- 34 | 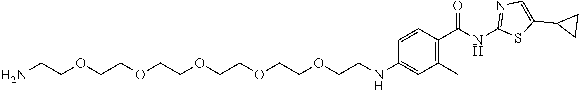 [SMILES: Cc1cc(NCCOCCOCCOCCOCCOCCN)ccc1C(=O)Nc1ncc(C2CC2)s1] | 4-((17-amino-3,6,9,12,15- pentaoxaheptadecyl) amino)-N-(5- cyclopropylthiazol-2-yl)- 2-methylbenzamide |
| BL1- 35 | 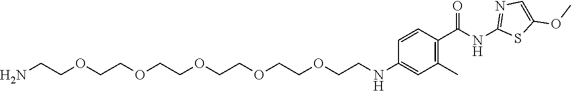 [SMILES: COc1cnc(NC(=O)c2ccc(NCCOCCOCCOCCOCCOCCN)cc2C)s1] | 4-((17-amino-3,6,9,12,15- pentaoxaheptadecyl) amino)-N-(5- methoxythiazol-2-yl)-2- methylbenzamide |
| BL1- 36 | 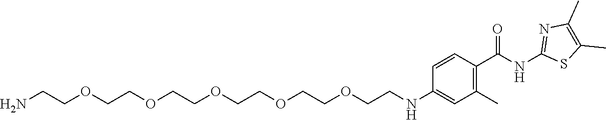 [SMILES: Cc1cc(NCCOCCOCCOCCOCCOCCN)ccc1C(=O)Nc1nc(C)c(C)s1] | 4-((17-amino-3,6,9,12,15- pentaoxaheptadecyl) amino)-N-(4,5- dimethylthiazol-2-yl)-2- methylbenzamide |
| BL1- 37 | 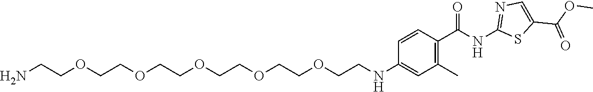 [SMILES: COC(=O)c1cnc(NC(=O)c2ccc(NCCOCCOCCOCCOCCOCCN)cc2C)s1] | methyl 2-(4-((17-amino- 3,6,9,12,15- pentaoxaheptadecyl) amino)-2- methylbenzamido)thiazole- 5-carboxylate |
| BL1- 38 | 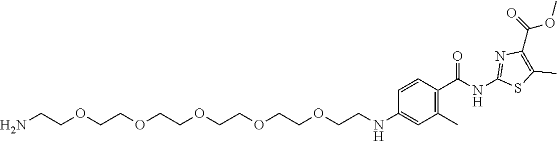 [SMILES: COC(=O)c1nc(NC(=O)c2ccc(NCCOCCOCCOCCOCCOCCN)cc2C)sc1C] | methyl 2-(4-((17-amino- 3,6,9,12,15- pentaoxaheptadecyl) amino)-2- methylbenzamido)-5- methylthiazole-4- carboxylate |
| BL1- 39 | 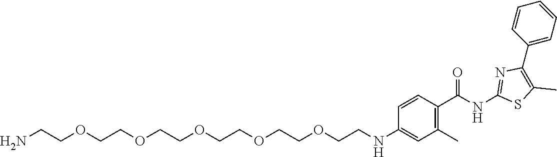 [SMILES: Cc1cc(NCCOCCOCCOCCOCCOCCN)ccc1C(=O)Nc1nc(-c2ccccc2)c(C)s1] | 4-((17-amino-3,6,9,12,15- pentaoxaheptadecyl) amino)-2-methyl-N- (5-methyl-4- phenylthiazol-2-yl) benzamide |
| BL1- 40 | 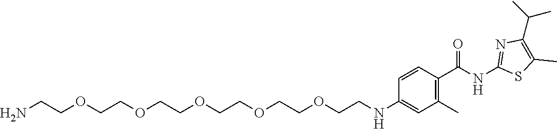 [SMILES: Cc1cc(NCCOCCOCCOCCOCCOCCN)ccc1C(=O)Nc1nc(C(C)C)c(C)s1] | 4-((17-amino-3,6,9,12,15- pentaoxaheptadecyl) amino)-N-(4-isopropyl- 5-methylthiazol-2- yl)-2-methylbenzamide |
| BL1- 41 | 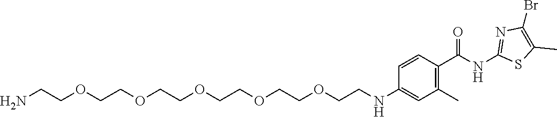 [SMILES: Cc1cc(NCCOCCOCCOCCOCCOCCN)ccc1C(=O)Nc1nc(Br)c(C)s1] | 4-((17-amino-3,6,9,12,15- pentaoxaheptadecyl)amino)- N-(4-bromo-5- methylthiazol-2- yl)-2-methylbenzamide |
| BL1- 42 | 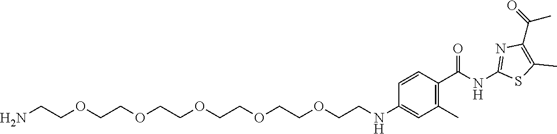 [SMILES: CC(=O)c1nc(NC(=O)c2ccc(NCCOCCOCCOCCOCCOCCN)cc2C)sc1C] | N-(4-acetyl-5- methylthiazol-2- yl)-4-((17-amino- 3,6,9,12,15- pentaoxaheptadecyl)amino)- 2-methylbenzamide |
| BL1- 43 | 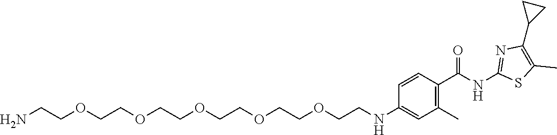 [SMILES: Cc1cc(NCCOCCOCCOCCOCCOCCN)ccc1C(=O)Nc1nc(C2CC2)c(C)s1] | 4-((17-amino-3,6,9,12,15- pentaoxaheptadecyl)amino)- N-(4-cyclopropyl-5- methylthiazol-2-yl)-2- methylbenzamide |
| BL1- 44 | 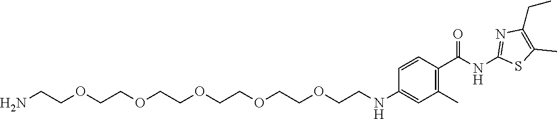 [SMILES: CCc1nc(NC(=O)c2ccc(NCCOCCOCCOCCOCCOCCN)cc2C)sc1C] | 4-((17-amino-3,6,9,12,15- pentaoxaheptadecyl) amino)-N-(4-ethyl-5- methylthiazol-2-yl)- 2-methylbenzamide |
| BL1- 45 | 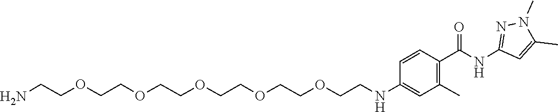 [SMILES: Cc1cc(NCCOCCOCCOCCOCCOCCN)ccc1C(=O)Nc1cc(C)n(C)n1] | 4-((17-amino-3,6,9,12,15- pentaoxaheptadecyl) amino)-N-(1,5-dimethyl- 1H-pyrazol-3- yl)-2-methylbenzamide |
| BL1- 46 | 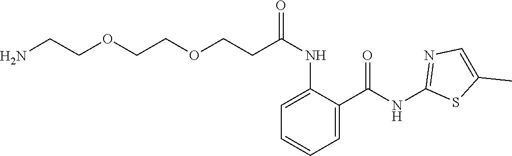 [SMILES: Cc1cnc(NC(=O)c2ccccc2NC(=O)CCOCCOCCN)s1] | 2-(3-(2-(2- aminoethoxy)ethoxy)propan- amido)-N-(5-methylthiazol-2- yl)benzamide |
| BL1- 47 | 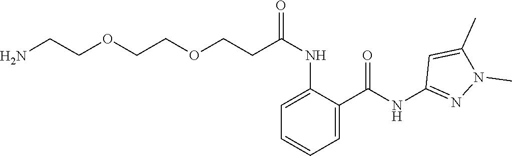 [SMILES: Cc1cc(NC(=O)c2ccccc2NC(=O)CCOCCOCCN)nn1C] | 2-(3-(2-(2- aminoethoxy)ethoxy)propan- amido)-N-(1,5-dimethyl-1H- pyrazol-3-yl)benzamide |
| BL1- 48 | 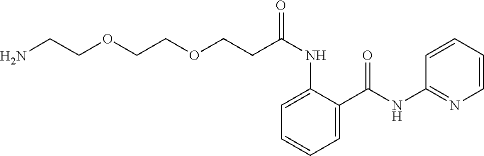 [SMILES: NCCOCCOCCC(=O)Nc1ccccc1C(=O)Nc1ccccn1] | 2-(3-(2-(2- aminoethoxy)ethoxy)propan- amido)-N-(72yridine-2- yl)benzamide |
| BL1- 49 | 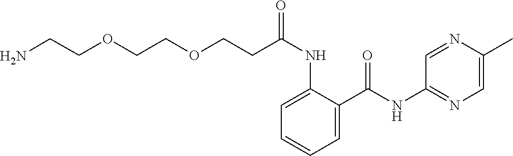 [SMILES: Cc1cnc(NC(=O)c2ccccc2NC(=O)CCOCCOCCN)cn1] | 2-(3-(2-(2- aminoethoxy)ethoxy)propan- amido)-N-(5-methylpyrazin- 2-yl)benzamide |
| BL1- 50 | 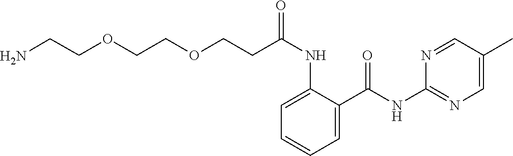 [SMILES: Cc1cnc(NC(=O)c2ccccc2NC(=O)CCOCCOCCN)nc1] | 2-(3-(2-(2- aminoethoxy)ethoxy) propanamido)-N-(5- methylpyrimidin- 2-yl)benzamide |
| BL1- 51 | 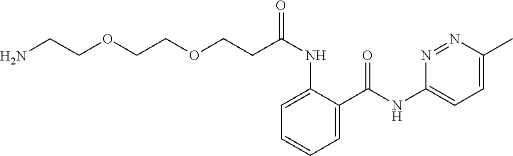 [SMILES: Cc1ccc(NC(=O)c2ccccc2NC(=O)CCOCCOCCN)nn1] | 2-(3-(2-(2- aminoethoxy)ethoxy) propanamido)-N-(6- methylpyridazin- 3-yl)benzamide |
| BL1- 52 | 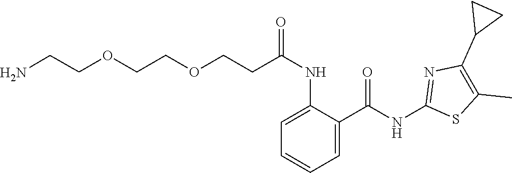 [SMILES: Cc1sc(NC(=O)c2ccccc2NC(=O)CCOCCOCCN)nc1C1CC1] | 2-(3-(2-(2- aminoethoxy)ethoxy)propan- amido)-N-(4-cyclopropyl-5- methylthiazol-2-yl)benzamide |
| BL1- 53 | 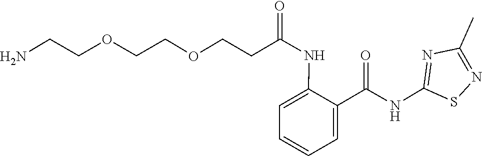 [SMILES: Cc1nsc(NC(=O)c2ccccc2NC(=O)CCOCCOCCN)n1] | 2-(3-(2-(2- aminoethoxy)ethoxy)propan- amido)-N-(3-methyl-1,2,4- thiadiazol-5-yl)benzamide |
| BL1- 54 | 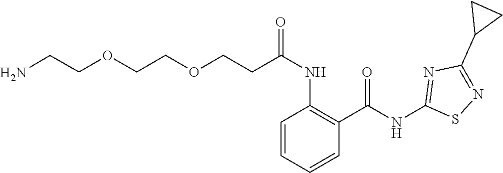 [SMILES: NCCOCCOCCC(=O)Nc1ccccc1C(=O)Nc1nc(C2CC2)ns1] | 2-(3-(2-(2- aminoethoxy)ethoxy)propan- amido)-N-(3- cyclopropyl-1,2,4- thiadiazol-5-yl)benzamide |
| BL1- 55 | 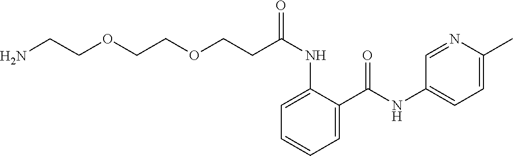 [SMILES: Cc1ccc(NC(=O)c2ccccc2NC(=O)CCOCCOCCN)cn1] | 2-(3-(2-(2- aminoethoxy)ethoxy)propan- amido)-N-(6-methylpyridin- 3-yl)benzamide |
| BL1- 56 | 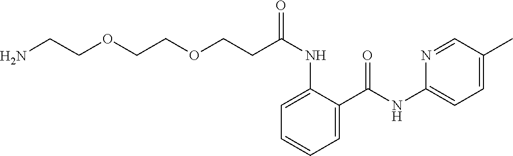 [SMILES: Cc1ccc(NC(=O)c2ccccc2NC(=O)CCOCCOCCN)nc1] | 2-(3-(2-(2- aminoethoxy)ethoxy)propan- amido)-N-(5-methylpyridin- 2-yl)benzamide |
| BL1- 57 | 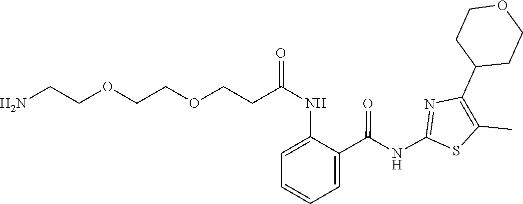 [SMILES: Cc1sc(NC(=O)c2ccccc2NC(=O)CCOCCOCCN)nc1C1CCOCC1] | 2-(3-(2-(2- aminoethoxy)ethoxy)propan- amido)-N-(5-methyl-4- (tetrahydro- 2H-pyran-4-yl)thiazol-2- yl)benzamide |
| BL1- 58 | 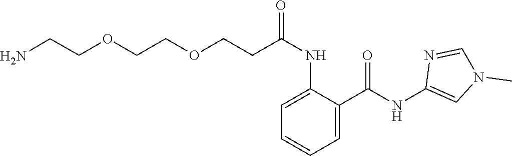 [SMILES: Cn1cnc(NC(=O)c2ccccc2NC(=O)CCOCCOCCN)c1] | 2-(3-(2-(2- aminoethoxy)ethoxy)propan- amido)-N-(1-methyl-1H- imidazol- 4-yl)benzamide |
| BL1- 59 | 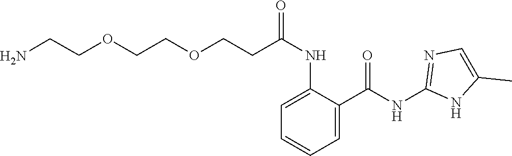 [SMILES: Cc1cnc(NC(=O)c2ccccc2NC(=O)CCOCCOCCN)[nH]1] | 2-(3-(2-(2- aminoethoxy)ethoxy)propan- amido)-N-(5-methyl-1H- imidazol- 2-yl)benzamide |
| BL1- 60 | 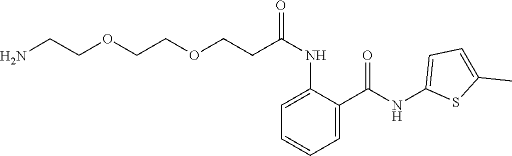 [SMILES: Cc1ccc(NC(=O)c2ccccc2NC(=O)CCOCCOCCN)s1] | 2-(3-(2-(2- aminoethoxy)ethoxy)propan- amido)-N-(5- methylthiophen-2- yl)benzamide |
| BL1- 61 | 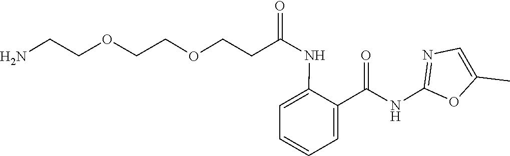 [SMILES: Cc1cnc(NC(=O)c2ccccc2NC(=O)CCOCCOCCN)o1] | 2-(3-(2-(2- aminoethoxy)ethoxy)propan- amido)-N-(5-methyloxazol-2- yl)benzamide |
| BL1- 62 | 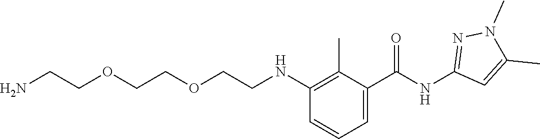 [SMILES: Cc1c(NCCOCCOCCN)cccc1C(=O)Nc1cc(C)n(C)n1] | 3-((2-(2-(2- aminoethoxy)ethoxy)ethyl) amino)-N-(1,5-dimethyl-1H- pyrazol-3-yl)-2- methylbenzamide |
| BL1- 63 | 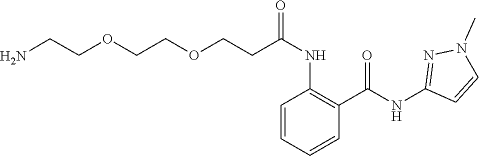 [SMILES: Cn1ccc(NC(=O)c2ccccc2NC(=O)CCOCCOCCN)n1] | 2-(3-(2-(2- aminoethoxy)ethoxy)propan- amido)-N-(1-methyl- 1H-pyrazol-3- yl)benzamide |
| BL1- 64 | 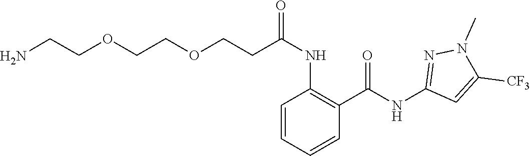 [SMILES: Cn1nc(NC(=O)c2ccccc2NC(=O)CCOCCOCCN)cc1C(F)(F)F] | 2-(3-(2-(2- aminoethoxy)ethoxy) propanamido)-N-(1-methyl-5- (trifluoromethyl)-1H-pyrazol- 3-yl)benzamide |
| BL1- 65 | 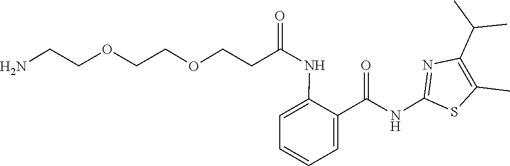 [SMILES: Cc1sc(NC(=O)c2ccccc2NC(=O)CCOCCOCCN)nc1C(C)C] | 2-(3-(2-(2- aminoethoxy)ethoxy)propan- amido)-N-(4-isopropyl-5- methylthiazol-2-yl)benzamide |
| BL1- 66 | 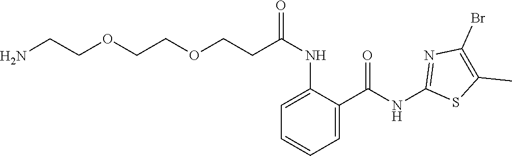 [SMILES: Cc1sc(NC(=O)c2ccccc2NC(=O)CCOCCOCCN)nc1Br] | 2-(3-(2-(2- aminoethoxy)ethoxy)propan- amido)-N-(4-bromo-5- methylthiazol-2-yl)benzamide |
| BL1- 67 | 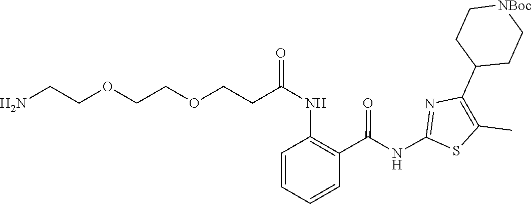 [SMILES: Cc1sc(NC(=O)c2ccccc2NC(=O)CCOCCOCCN)nc1C1CCN(C(=O)OC(C)(C)C)CC1] | 2-(3-(2-(2- aminoethoxy)ethoxy) propanamido)- N-(5-methyl-4-(piperidin-4- yl)thiazol-2-yl)benzamide |
| BL1- 68 | 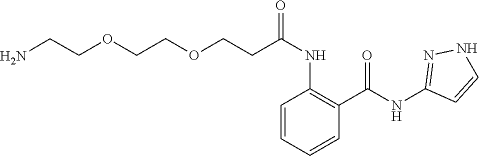 [SMILES: NCCOCCOCCC(=O)Nc1ccccc1C(=O)Nc1cc[nH]n1] | 2-(3-(2-(2- aminoethoxy)ethoxy)propan- amido)-N-(1H-pyrazol-3- yl)benzamide |
| BL1- 69 | 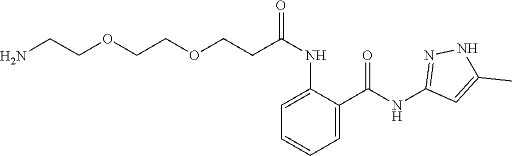 [SMILES: Cc1cc(NC(=O)c2ccccc2NC(=O)CCOCCOCCN)n[nH]1] | 2-(3-(2-(2- aminoethoxy)ethoxy)propan- amido)-N-(5-methyl-1H- pyrazol-3- yl)benzamide |
| BL1- 70 | 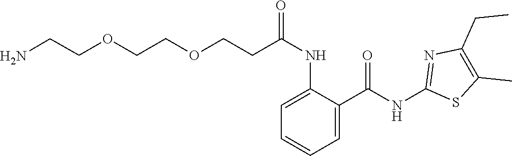 [SMILES: CCc1nc(NC(=O)c2ccccc2NC(=O)CCOCCOCCN)sc1C] | 2-(3-(2-(2- aminoethoxy)ethoxy)propan- amido)-N-(4-ethyl-5- methylthiazol- 2-yl)benzamide |
| BL1- 71 | 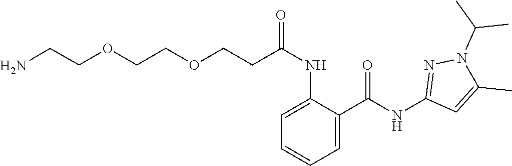 [SMILES: Cc1cc(NC(=O)c2ccccc2NC(=O)CCOCCOCCN)nn1C(C)C] | 2-(3-(2-(2- aminoethoxy)ethoxy)propan- amido)-N-(1-isopropyl- 5-methyl- 1H-pyrazol-3-yl)benzamide |
| BL1- 72 | 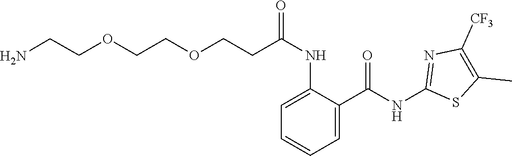 [SMILES: Cc1sc(NC(=O)c2ccccc2NC(=O)CCOCCOCCN)nc1C(F)(F)F] | 2-(3-(2-(2- aminoethoxy)ethoxy)propan- amido)-N-(5-methyl-4- (trifluoromethyl)thiazol-2- yl)benzamide |
| BL1- 73 | 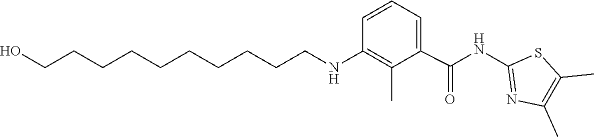 [SMILES: Cc1nc(NC(=O)c2cccc(NCCCCCCCCCCO)c2C)sc1C] | N-(4,5-dimethylthiazol- 2-yl)-3-((10- hydroxydecyl)amino)-2- methylbenzamide |
| BL1- 74 | 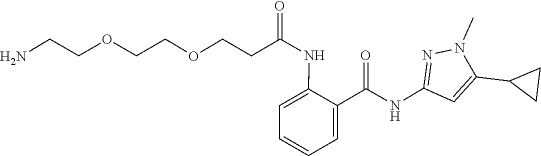 [SMILES: Cn1nc(NC(=O)c2ccccc2NC(=O)CCOCCOCCN)cc1C1CC1] | 2-(3-(2-(2- aminoethoxy)ethoxy)propan- amido)-N-(5-cyclopropyl- 1-methyl- 1H-pyrazol-3-yl)benzamide |
| BL1- 75 | 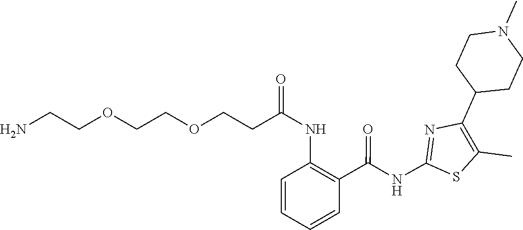 [SMILES: Cc1sc(NC(=O)c2ccccc2NC(=O)CCOCCOCCN)nc1C1CCN(C)CC1] | 2-(3-(2-(2- aminoethoxy)ethoxy)propan- amido)-N-(5-methyl-4-(1- methylpiperidin-4-yl)thiazol- 2-yl)benzamide |
| BL1- 76 | 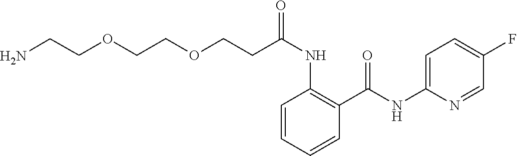 [SMILES: NCCOCCOCCC(=O)Nc1ccccc1C(=O)Nc1ccc(F)cn1] | 2-(3-(2-(2- aminoethoxy)ethoxy)propan- amido)-N-(5-fluoropyridin-2- yl)benzamide |
| BL1- 77 | 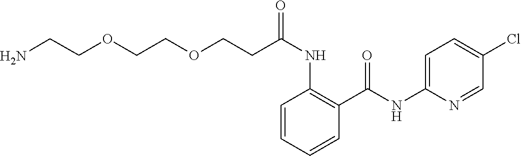 [SMILES: NCCOCCOCCC(=O)Nc1ccccc1C(=O)Nc1ccc(Cl)cn1] | 2-(3-(2-(2- aminoethoxy)ethoxy)propan- amido)-N-(5-chloropyridin-2- yl)benzamide |
| BL1- 78 | 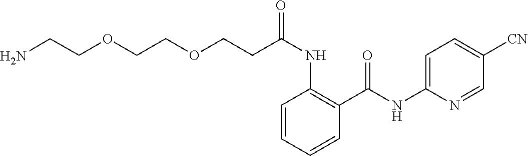 [SMILES: N#Cc1ccc(NC(=O)c2ccccc2NC(=O)CCOCCOCCN)nc1] | 2-(3-(2-(2- aminoethoxy)ethoxy)propan- amido)-N-(5-cyanopyridin-2- yl)benzamide |
| BL1- 79 | 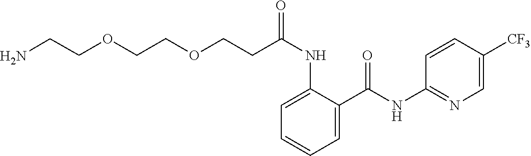 [SMILES: NCCOCCOCCC(=O)Nc1ccccc1C(=O)Nc1ccc(C(F)(F)F)cn1] | 2-(3-(2-(2- aminoethoxy)ethoxy)propan- amido)-N-(5- (trifluoromethyl)74yridine-2- yl)benzamide |
| BL1- 80 | 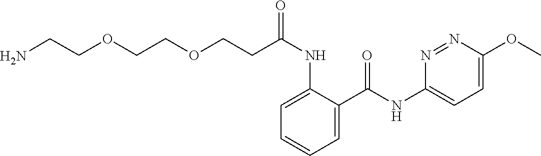 [SMILES: COc1ccc(NC(=O)c2ccccc2NC(=O)CCOCCOCCN)nn1] | 2-(3-(2-(2- aminoethoxy)ethoxy)propan- amido)-N-(6- methoxypyridazin-3- yl)benzamide |
| BL1- 81 | 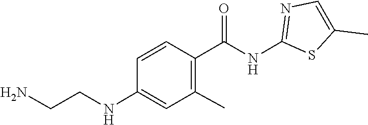 [SMILES: Cc1cnc(NC(=O)c2ccc(NCCN)cc2C)s1] | 4-((2-aminoethyl)amino)-2- methyl-N-(5-methylthiazol-2- yl)benzamide |
| BL1- 82 | 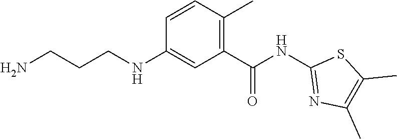 [SMILES: Cc1ccc(NCCCN)cc1C(=O)Nc1nc(C)c(C)s1] | 5-((3-aminopropyl)amino)-N- (4,5-dimethylthiazol-2-yl)-2- methylbenzamide |
| BL1- 83 | 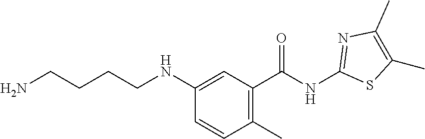 [SMILES: Cc1ccc(NCCCCN)cc1C(=O)Nc1nc(C)c(C)s1] | 5-((4-aminobutyl)amino)-N- (4,5-dimethylthiazol-2-yl)-2- methylbenzamide |
| BL1- 84 | 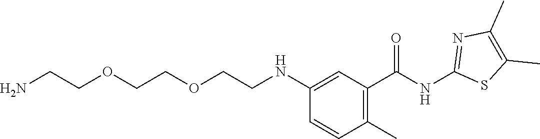 [SMILES: Cc1ccc(NCCOCCOCCN)cc1C(=O)Nc1nc(C)c(C)s1] | 5-((2-(2-(2- aminoethoxy)ethoxy)ethyl) amino)-N-(4,5- dimethylthiazol-2- yl)-2-methylbenzamide |
| BL1- 85 | 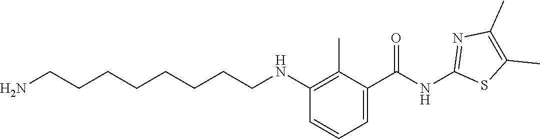 [SMILES: Cc1nc(NC(=O)c2cccc(NCCCCCCCCN)c2C)sc1C] | 3-((8-aminooctyl)amino)-N- (4,5-dimethylthiazol-2-yl)-2- methylbenzamide |
| BL1- 86 | 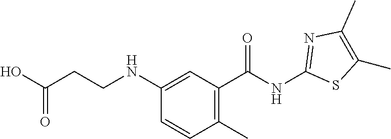 [SMILES: Cc1ccc(NCCC(=O)O)cc1C(=O)Nc1nc(C)c(C)s1] | 3-((3-((4,5-dimethylthiazol-2- yl)carbamoyl)-4- methylphenyl)amino) propanoic acid |
| BL1- 87 | 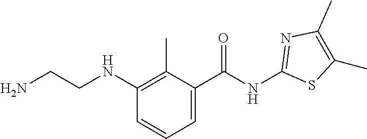 [SMILES: Cc1nc(NC(=O)c2cccc(NCCN)c2C)sc1C] | 3-((2-aminoethyl)amino)-N- (4,5-dimethylthiazol-2-yl)-2- methylbenzamide |
| BL1- 88 | 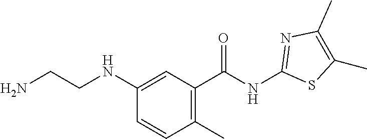 [SMILES: Cc1ccc(NCCN)cc1C(=O)Nc1nc(C)c(C)s1] | 5-((2-aminoethyl)amino)-N- (4,5-dimethylthiazol-2-yl)-2- methylbenzamide |
| BL1- 89 | 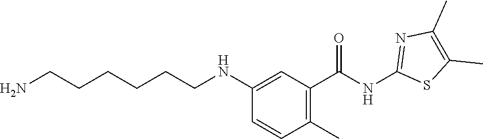 [SMILES: Cc1ccc(NCCCCCCN)cc1C(=O)Nc1nc(C)c(C)s1] | 5-((6-aminohexyl)amino)-N- (4,5-dimethylthiazol-2-yl)-2- methylbenzamide |
| BL1- 90 | 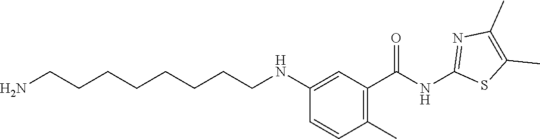 [SMILES: Cc1ccc(NCCCCCCCCN)cc1C(=O)Nc1nc(C)c(C)s1] | 5-((8-aminooctyl)amino)-N- (4,5-dimethylthiazol-2-yl)-2- methylbenzamide |
| BL1- 91 | 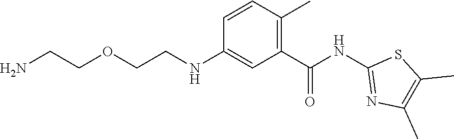 [SMILES: Cc1ccc(NCCOCCN)cc1C(=O)Nc1nc(C)c(C)s1] | 5-((2-(2- aminoethoxy)ethyl)amino)-N- (4,5-dimethylthiazol-2-yl)-2- methylbenzamide |
| BL1- 92 | 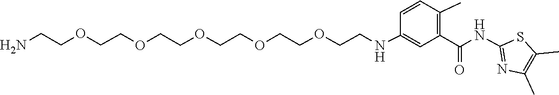 [SMILES: Cc1ccc(NCCOCCOCCOCCOCCOCCN)cc1C(=O)Nc1nc(C)c(C)s1] | 5-((17-amino-3,6,9,12,15- pentaoxaheptadecyl) amino)-N-(4,5- dimethylthiazol-2-yl)-2- methylbenzamide |
| BL1- 93 | 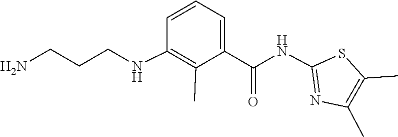 [SMILES: Cc1nc(NC(=O)c2cccc(NCCCN)c2C)sc1C] | 3-((3-aminopropyl)amino)-N- (4,5-dimethylthiazol-2-yl)-2- methylbenzamide |
| BL1- 94 | 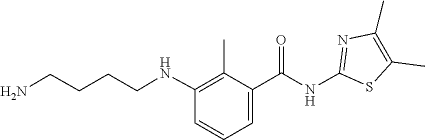 [SMILES: Cc1nc(NC(=O)c2cccc(NCCCCN)c2C)sc1C] | 3-((4-aminobutyl)amino)-N- (4,5-dimethylthiazol-2-yl)-2- methylbenzamide |
| BL1- 95 | 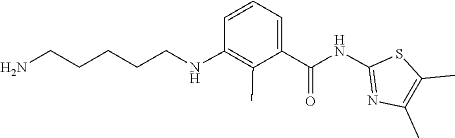 [SMILES: Cc1nc(NC(=O)c2cccc(NCCCCCN)c2C)sc1C] | 3-((5-aminopentyl)amino)-N- (4,5-dimethylthiazol-2-yl)-2- methylbenzamide |
| BL1- 96 | 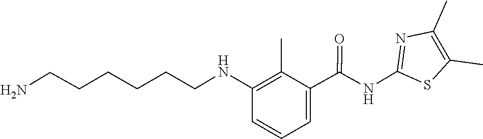 [SMILES: Cc1nc(NC(=O)c2cccc(NCCCCCCN)c2C)sc1C] | 3-((6-aminohexyl)amino)-N- (4,5-dimethylthiazol-2-yl)-2- methylbenzamide |
| BL1- 97 | 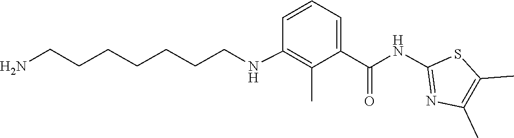 [SMILES: Cc1nc(NC(=O)c2cccc(NCCCCCCCN)c2C)sc1C] | 3-((7-aminoheptyl)amino)-N- (4,5-dimethylthiazol-2-yl)-2- methylbenzamide |
| BL1- 98 | 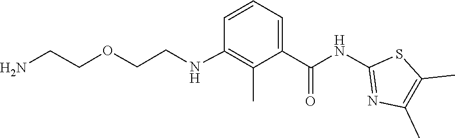 [SMILES: Cc1nc(NC(=O)c2cccc(NCCOCCN)c2C)sc1C] | 3-((2-(2- aminoethoxy)ethyl)amino)-N- (4,5-dimethylthiazol-2-yl)-2- methylbenzamide |
| BL1- 99 | 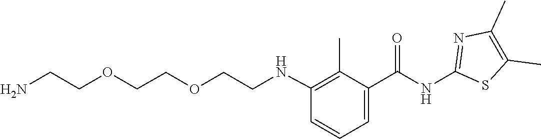 [SMILES: Cc1nc(NC(=O)c2cccc(NCCOCCOCCN)c2C)sc1C] | 3-((2-(2-(2- aminoethoxy)ethoxy)ethyl) amino)-N-(4,5- dimethylthiazol-2- yl)-2-methylbenzamide |
| BL1- 100 | 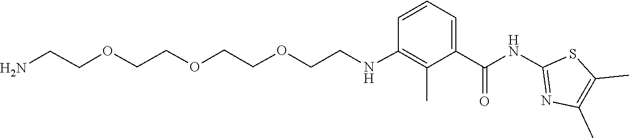 [SMILES: Cc1nc(NC(=O)c2cccc(NCCOCCOCCOCCN)c2C)sc1C] | 3-((2-(2-(2-(2- aminoethoxy)ethoxy)ethoxy) ethyl)amino)-N-(4,5- dimethylthiazol-2-yl)-2- methylbenzamide |
| BL1- 101 | 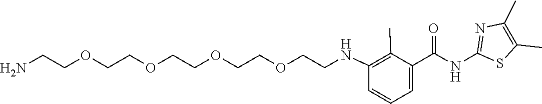 [SMILES: Cc1nc(NC(=O)c2cccc(NCCOCCOCCOCCOCCN)c2C)sc1C] | 3-((14-amino-3,6,9,12- tetraoxatetradecyl)amino)-N- (4,5-dimethylthiazol-2-yl)-2- methylbenzamide |
| BL1- 102 | 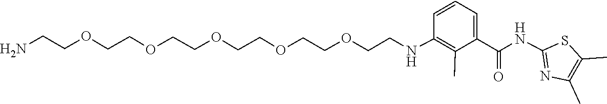 [SMILES: Cc1nc(NC(=O)c2cccc(NCCOCCOCCOCCOCCOCCN)c2C)sc1C] | 3-((17-amino-3,6,9,12,15- pentaoxaheptadecyl) amino)-N-(4,5- dimethylthiazol-2-yl)-2- methylbenzamide |
| BL1- 103 | 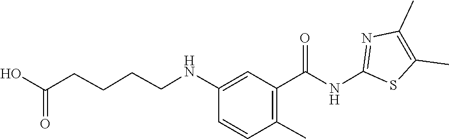 [SMILES: Cc1ccc(NCCCCC(=O)O)cc1C(=O)Nc1nc(C)c(C)s1] | 5-((3-((4,5-dimethylthiazol-2- yl)carbamoyl)-4- methylphenyl)amino) pentanoic acid |
| BL1- 104 | 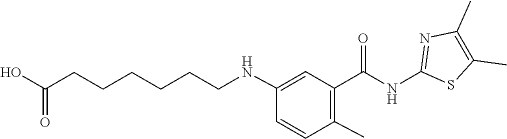 [SMILES: Cc1ccc(NCCCCCCC(=O)O)cc1C(=O)Nc1nc(C)c(C)s1] | 7-((3-((4,5-dimethylthiazol-2- yl)carbamoyl)-4- methylphenyl)amino) heptanoic acid |
| BL1- 105 | 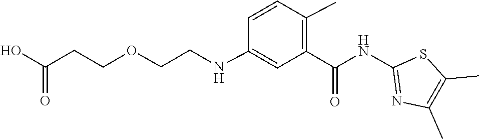 [SMILES: Cc1ccc(NCCOCCC(=O)O)cc1C(=O)Nc1nc(C)c(C)s1] | 3-(2-((3-((4,5- dimethylthiazol- 2-yl)carbamoyl)-4- methylphenyl)amino)ethoxy) propanoic acid |
| BL1- 106 | 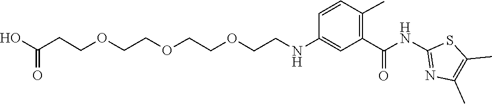 [SMILES: Cc1ccc(NCCOCCOCCOCCC(=O)O)cc1C(=O)Nc1nc(C)c(C)s1] | 3-(2-(2-(2-((3-((4,5- dimethylthiazol-2- yl)carbamoyl)-4- methylphenyl)amino)ethoxy) ethoxy)ethoxy)propanoic acid |
| BL1- 107 | 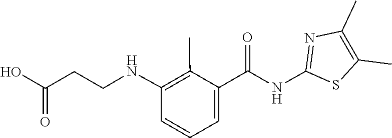 [SMILES: Cc1nc(NC(=O)c2cccc(NCCC(=O)O)c2C)sc1C] | 3-((3-((4,5-dimethylthiazol-2- yl)carbamoyl)-2- methylphenyl)amino) propanoic acid |
| BL1- 108 | 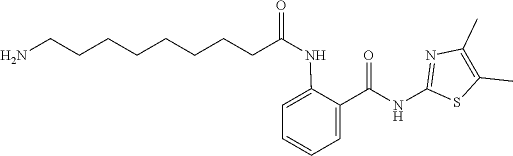 [SMILES: Cc1nc(NC(=O)c2ccccc2NC(=O)CCCCCCCCN)sc1C] | 2-(9-aminononanamido)-N- (4,5-dimethylthiazol-2- yl)benzamide |
| BL1- 109 | 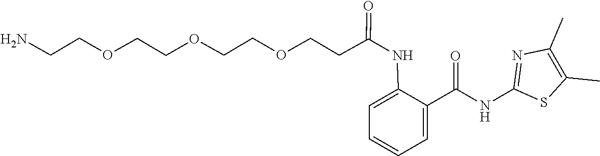 [SMILES: Cc1nc(NC(=O)c2ccccc2NC(=O)CCOCCOCCOCCN)sc1C] | 2-(3-(2-(2-(2- aminoethoxy)ethoxy)ethoxy) propanamido)-N-(4,5- dimethylthiazol-2-yl) benzamide |
| BL1- 110 | 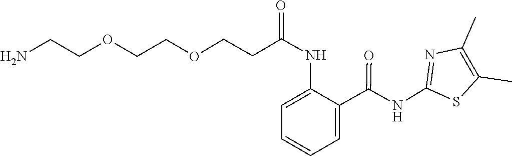 [SMILES: Cc1nc(NC(=O)c2ccccc2NC(=O)CCOCCOCCN)sc1C] | 2-(3-(2-(2- aminoethoxy)ethoxy)propan- amido)-N-(4,5- dimethylthiazol-2- yl)benzamide |
| BL1- 111 | 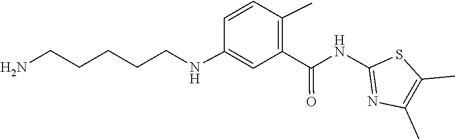 [SMILES: Cc1ccc(NCCCCCN)cc1C(=O)Nc1nc(C)c(C)s1] | 5-((5-aminopentyl)amino)-N- (4,5-dimethylthiazol-2-yl)-2- methylbenzamide |
| BL1- 112 | 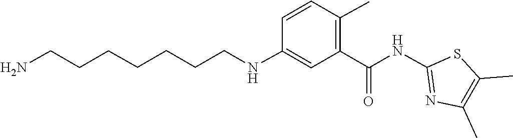 [SMILES: Cc1ccc(NCCCCCCCN)cc1C(=O)Nc1nc(C)c(C)s1] | 5-((7-aminoheptyl)amino)-N- (4,5-dimethylthiazol-2-yl)-2- methylbenzamide |
| BL1- 113 | 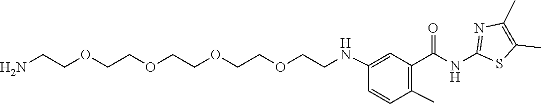 [SMILES: Cc1ccc(NCCOCCOCCOCCOCCN)cc1C(=O)Nc1nc(C)c(C)s1] | 5-((14-amino-3,6,9,12- tetraoxatetradecyl)amino)-N- (4,5-dimethylthiazol-2-yl)-2- methylbenzamide |
| BL1- 114 | 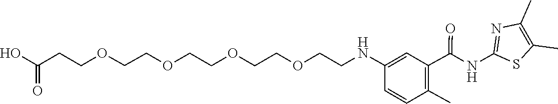 [SMILES: Cc1ccc(NCCOCCOCCOCCOCCC(=O)O)cc1C(=O)Nc1nc(C)c(C)s1] | 1-((3-((4,5-dimethylthiazol-2- yl)carbamoyl)-4- methylphenyl)amino)- 3,6,9,12- tetraoxapentadecan- 15-oic acid |
| BL1- 115 | 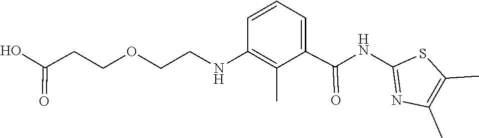 [SMILES: Cc1nc(NC(=O)c2cccc(NCCOCCC(=O)O)c2C)sc1C] | 3-(2-((3-((4,5- dimethylthiazol- 2-yl)carbamoyl)-2- methylphenyl)amino)ethoxy) propanoic acid |
| BL1- 116 | 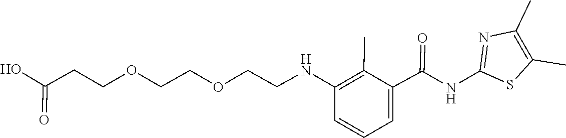 [SMILES: Cc1nc(NC(=O)c2cccc(NCCOCCOCCC(=O)O)c2C)sc1C] | 3-(2-(2-((3-((4,5- dimethylthiazol-2- yl)carbamoyl)-2- methylphenyl)amino)ethoxy) ethoxy)propanoic acid |
| BL1- 117 | 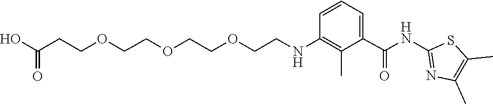 [SMILES: Cc1nc(NC(=O)c2cccc(NCCOCCOCCOCCC(=O)O)c2C)sc1C] | 3-(2-(2-(2-((3-((4,5- dimethylthiazol-2- yl)carbamoyl)-2- methylphenyl)amino) ethoxy)ethoxy)ethoxy) propanoic acid |
| BL1- 118 | 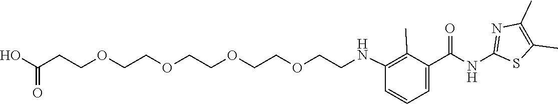 [SMILES: Cc1nc(NC(=O)c2cccc(NCCOCCOCCOCCOCCC(=O)O)c2C)sc1C] | 1-((3-((4,5-dimethylthiazol- 2-yl)carbamoyl)-2- methylphenyl)amino)- 3,6,9,12- tetraoxapentadecan- 15-oic acid |
| BL1- 119 | 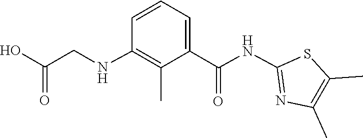 [SMILES: Cc1nc(NC(=O)c2cccc(NCC(=O)O)c2C)sc1C] | (3-((4,5-dimethylthiazol-2- yl)carbamoyl)-2- methylphenyl)glycine |
| BL1- 120 | 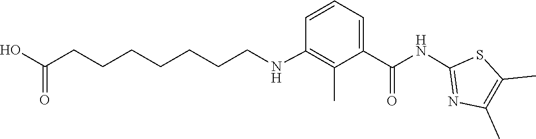 [SMILES: Cc1nc(NC(=O)c2cccc(NCCCCCCCC(=O)O)c2C)sc1C] | 8-((3-((4,5-dimethylthiazol- 2-yl)carbamoyl)-2- methylphenyl)amino) octanoic acid |
| BL1- 121 | 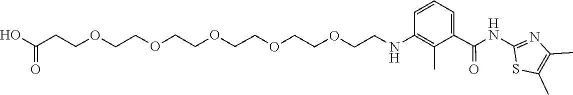 [SMILES: Cc1nc(NC(=O)c2cccc(NCCOCCOCCOCCOCCOCCC(=O)O)c2C)sc1C] | 1-((3-((4,5-dimethylthiazol- 2-yl)carbamoyl)-2- methylphenyl)amino)- 3,6,9,12,15- pentaoxaoctadecan- 18-oic acid |
| BL1- 122 | 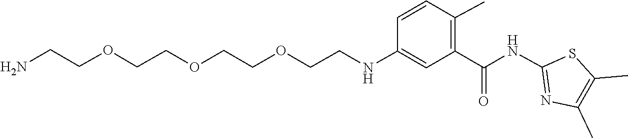 [SMILES: Cc1ccc(NCCOCCOCCOCCN)cc1C(=O)Nc1nc(C)c(C)s1] | 5-((2-(2-(2-(2- aminoethoxy)ethoxy)ethoxy) ethyl)amino)-N-(4,5- dimethylthiazol-2-yl)-2- methylbenzamide |
| BL1- 123 | 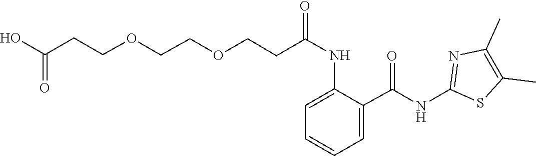 [SMILES: Cc1nc(NC(=O)c2ccccc2NC(=O)CCOCCOCCC(=O)O)sc1C] | 3-(2-(3-((2-((4,5- dimethylthiazol-2-yl) carbamoyl)phenyl)amino)-3- oxopropoxy)ethoxy) propanoic acid |
| BL1- 124 | 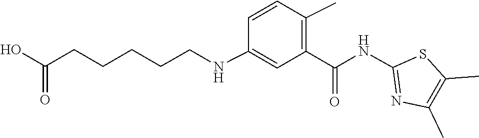 [SMILES: Cc1ccc(NCCCCCC(=O)O)cc1C(=O)Nc1nc(C)c(C)s1] | 6-((3-((4,5-dimethylthiazol- 2-yl)carbamoyl)-4- methylphenyl)amino) hexanoic acid |
| BL1- 125 | 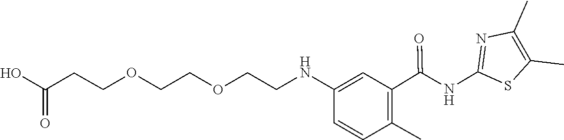 [SMILES: Cc1ccc(NCCOCCOCCC(=O)O)cc1C(=O)Nc1nc(C)c(C)s1] | 3-(2-(2-((3-((4,5- dimethylthiazol-2- yl)carbamoyl)-4- methylphenyl)amino)ethoxy) ethoxy)propanoic acid |
| BL1- 126 | 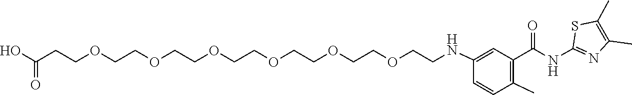 [SMILES: Cc1ccc(NCCOCCOCCOCCOCCOCCOCCC(=O)O)cc1C(=O)Nc1nc(C)c(C)s1] | 1-((3-((4,5-dimethylthiazol-2- yl)carbamoyl)-4- methylphenyl)amino)- 3,6,9,12,15,18- hexaoxahenicosan-21- oic acid |
| BL1- 127 | 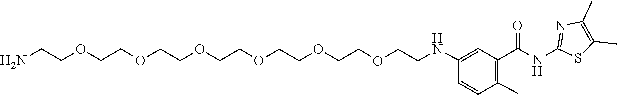 [SMILES: Cc1ccc(NCCOCCOCCOCCOCCOCCOCCN)cc1C(=O)Nc1nc(C)c(C)s1] | 5-((20-amino-3,6,9,12,15,18- hexaoxaicosyl)amino)- N-(4,5- dimethylthiazol-2-yl)-2- methylbenzamide |
| BL1- 128 | 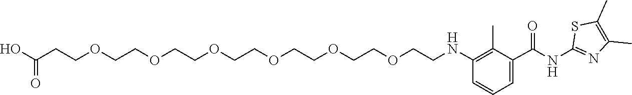 [SMILES: Cc1nc(NC(=O)c2cccc(NCCOCCOCCOCCOCCOCCOCCC(=O)O)c2C)sc1C] | 1-((3-((4,5-dimethylthiazol- 2-yl)carbamoyl)-2- methylphenyl)amino)- 3,6,9,12,15,18- hexaoxahenicosan-21- oic acid |
| BL1- 129 | 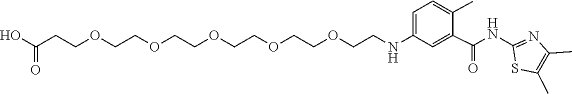 [SMILES: Cc1ccc(NCCOCCOCCOCCOCCOCCC(=O)O)cc1C(=O)Nc1nc(C)c(C)s1] | 1-((3-((4,5-dimethylthiazol- 2-yl)carbamoyl)-4- methylphenyl)amino)- 3,6,9,12,15- pentaoxaoctadecan- 18-oic acid |
| BL1- 130 | 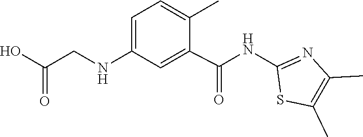 [SMILES: Cc1ccc(NCC(=O)O)cc1C(=O)Nc1nc(C)c(C)s1] | (3-((4,5-dimethylthiazol-2- yl)carbamoyl)-4- methylphenyl)glycine |
| BL1- 131 | 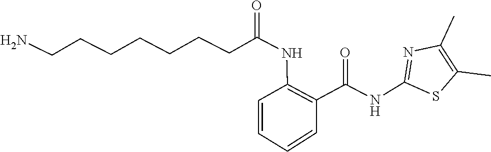 [SMILES: Cc1nc(NC(=O)c2ccccc2NC(=O)CCCCCCCN)sc1C] | 2-(8-aminooctanamido)- N-(4,5-dimethylthiazol- 2-yl)benzamide |
| BL1- 132 | 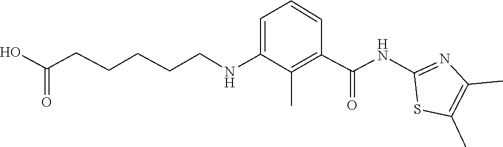 [SMILES: Cc1nc(NC(=O)c2cccc(NCCCCCC(=O)O)c2C)sc1C] | 6-((3-((4,5-dimethylthiazol- 2-yl)carbamoyl)-2- methylphenyl)amino) hexanoic acid |
| BL1- 133 | 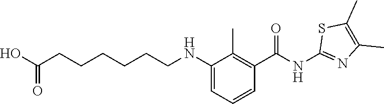 [SMILES: Cc1nc(NC(=O)c2cccc(NCCCCCCC(=O)O)c2C)sc1C] | 7-((3-((4,5-dimethylthiazol- 2-yl)carbamoyl)-2- methylphenyl)amino) heptanoic acid |
| BL1- 134 | 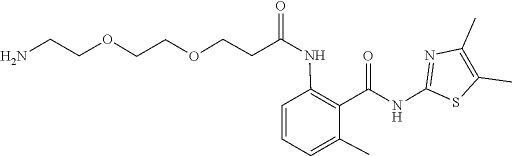 [SMILES: Cc1cccc(NC(=O)CCOCCOCCN)c1C(=O)Nc1nc(C)c(C)s1] | 2-(3-(2-(2-aminoethoxy) ethoxy)propanamido)- N-(4,5-dimethylthiazol-2- yl)-6-methylbenzamide |
| BL1- 135 | 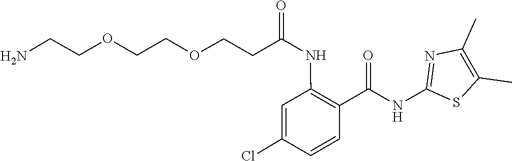 [SMILES: Cc1nc(NC(=O)c2ccc(Cl)cc2NC(=O)CCOCCOCCN)sc1C] | 2-(3-(2-(2-aminoethoxy) ethoxy)propanamido)- 4-chloro-N-(4,5- dimethylthiazol-2-yl) benzamide |
| BL1- 136 | 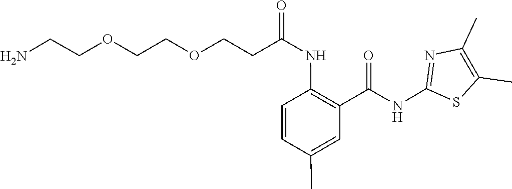 [SMILES: Cc1ccc(NC(=O)CCOCCOCCN)c(C(=O)Nc2nc(C)c(C)s2)c1] | 2-(3-(2-(2-aminoethoxy) ethoxy)propanamido)- N-(4,5-dimethylthiazol-2- yl)-5-methylbenzamide |
| BL1- 137 | 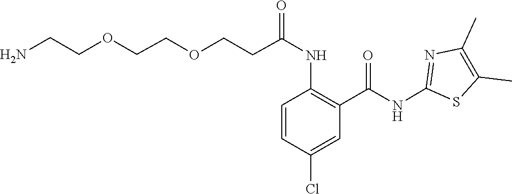 [SMILES: Cc1nc(NC(=O)c2cc(Cl)ccc2NC(=O)CCOCCOCCN)sc1C] | 2-(3-(2-(2-aminoethoxy) ethoxy)propanamido)- 5-chloro-N-(4,5- dimethylthiazol-2-yl) benzamide |
| BL1- 138 | 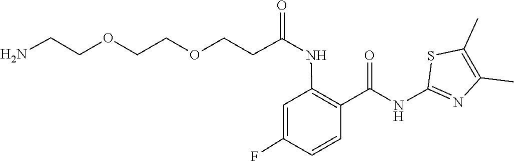 [SMILES: Cc1nc(NC(=O)c2ccc(F)cc2NC(=O)CCOCCOCCN)sc1C] | 2-(3-(2-(2-aminoethoxy) ethoxy)propanamido)- N-(4,5-dimethylthiazol-2- yl)-4-fluorobenzamide |
| BL1- 139 | 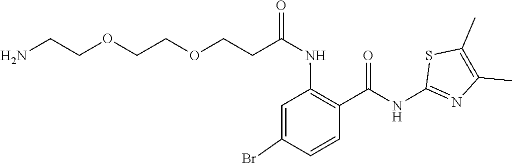 [SMILES: Cc1nc(NC(=O)c2ccc(Br)cc2NC(=O)CCOCCOCCN)sc1C] | 2-(3-(2-(2-aminoethoxy) ethoxy)propanamido)- 4-bromo-N-(4,5- dimethylthiazol-2-yl) benzamide |
| BL1- 140 | 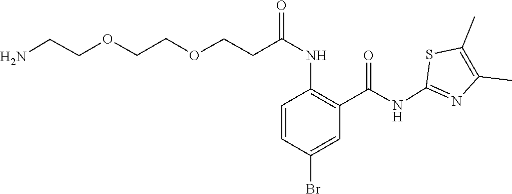 [SMILES: Cc1nc(NC(=O)c2cc(Br)ccc2NC(=O)CCOCCOCCN)sc1C] | 2-(3-(2-(2-aminoethoxy) ethoxy)propanamido)- 5-bromo-N-(4,5- dimethylthiazol-2-yl) benzamide |
| BL1- 141 | 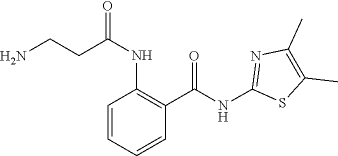 [SMILES: Cc1nc(NC(=O)c2ccccc2NC(=O)CCN)sc1C] | 2-(3-aminopropanamido)-N- (4,5-dimethylthiazol-2- yl)benzamide |
| BL1- 142 | 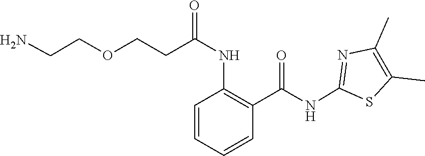 [SMILES: Cc1nc(NC(=O)c2ccccc2NC(=O)CCOCCN)sc1C] | 2-(3-(2-aminoethoxy) propanamido)-N- (4,5-dimethylthiazol-2- yl)benzamide |
| BL1- 143 | 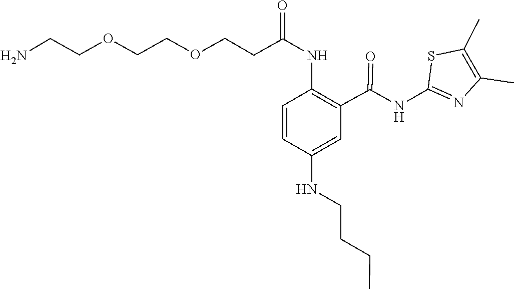 [SMILES: CCCCNc1ccc(NC(=O)CCOCCOCCN)c(C(=O)Nc2nc(C)c(C)s2)c1] | 2-(3-(2-(2-aminoethoxy) ethoxy)propanamido)- 5-(butylamino)-N-(4,5- dimethylthiazol-2-yl) benzamide |
| BL1- 144 | 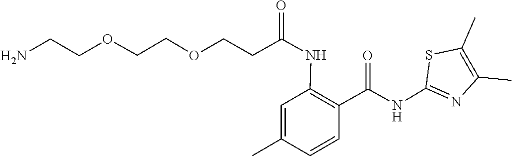 [SMILES: Cc1ccc(C(=O)Nc2nc(C)c(C)s2)c(NC(=O)CCOCCOCCN)c1] | 2-(3-(2-(2-aminoethoxy) ethoxy)propanamido)- N-(4,5-dimethylthiazol-2- yl)-4-methylbenzamide |
| BL1- 145 | 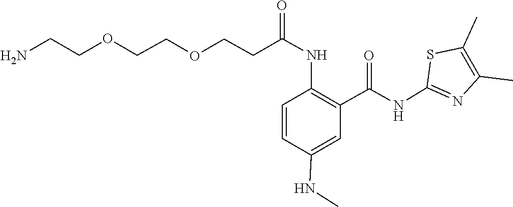 [SMILES: CNc1ccc(NC(=O)CCOCCOCCN)c(C(=O)Nc2nc(C)c(C)s2)c1] | 2-(3-(2-(2-aminoethoxy) ethoxy)propanamido)- N-(4,5-dimethylthiazol-2- yl)-5-(methylamino) benzamide |
| BL1- 146 | 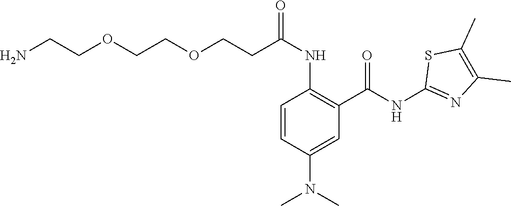 [SMILES: Cc1nc(NC(=O)c2cc(N(C)C)ccc2NC(=O)CCOCCOCCN)sc1C] | 2-(3-(2-(2-aminoethoxy) ethoxy)propanamido)- 5-(dimethylamino)-N-(4,5- dimethylthiazol-2-yl) benzamide |
| BL1- 147 | 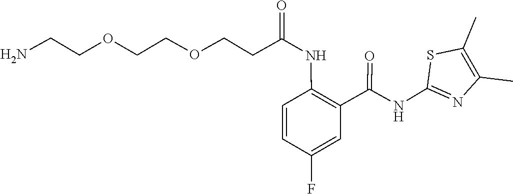 [SMILES: Cc1nc(NC(=O)c2cc(F)ccc2NC(=O)CCOCCOCCN)sc1C] | 2-(3-(2-(2-aminoethoxy) ethoxy)propanamido)- N-(4,5-dimethylthiazol-2- yl)-5-fluorobenzamide |
| BL1- 148 | 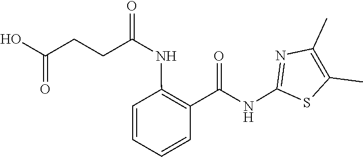 [SMILES: Cc1nc(NC(=O)c2ccccc2NC(=O)CCC(=O)O)sc1C] | 4-((2-((4,5- dimethylthiazol-2- yl)carbamoyl)phenyl) amino)-4-oxobutanoic acid |
| BL1- 149 | 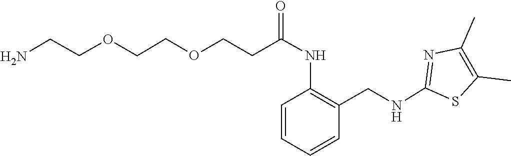 [SMILES: Cc1nc(NCc2ccccc2NC(=O)CCOCCOCCN)sc1C] | 3-(2-(2-aminoethoxy) ethoxy)-N-(2-(((4,5- dimethylthiazol-2- yl)amino)methyl)phenyl) propanamide |
| BL1- 150 | 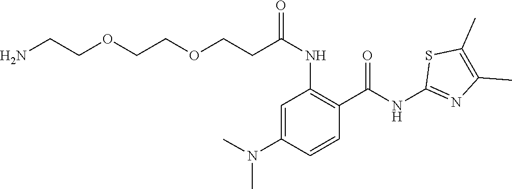 [SMILES: Cc1nc(NC(=O)c2ccc(N(C)C)cc2NC(=O)CCOCCOCCN)sc1C] | 2-(3-(2-(2-aminoethoxy) ethoxy)propanamido)- 4-(dimethylamino)-N-(4,5- dimethylthiazol-2-yl) benzamide |
| BL1- 151 | 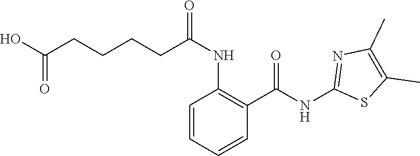 [SMILES: Cc1nc(NC(=O)c2ccccc2NC(=O)CCCCC(=O)O)sc1C] | 6-((2-((4,5- dimethylthiazol-2- yl)carbamoyl)phenyl) amino)-6-oxohexanoic acid |
| BL1- 152 | 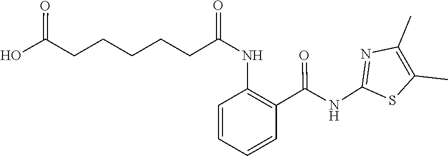 [SMILES: Cc1nc(NC(=O)c2ccccc2NC(=O)CCCCCC(=O)O)sc1C] | 7-((2-((4,5- dimethylthiazol-2- yl)carbamoyl)phenyl) amino)-7-oxoheptanoic acid |
| BL1- 153 | 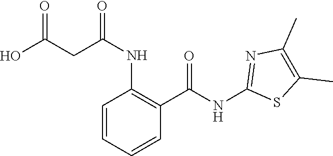 [SMILES: Cc1nc(NC(=O)c2ccccc2NC(=O)CC(=O)O)sc1C] | 3-((2-((4,5- dimethylthiazol-2- yl)carbamoyl)phenyl) amino)-3-oxopropanoic acid |
| BL1- 154 | 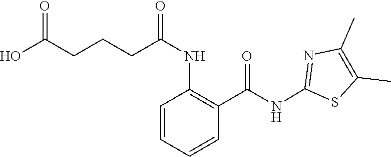 [SMILES: Cc1nc(NC(=O)c2ccccc2NC(=O)CCCC(=O)O)sc1C] | 5-((2-((4,5- dimethylthiazol-2- yl)carbamoyl)phenyl) amino)-5-oxopentanoic acid |
| BL1- 155 | 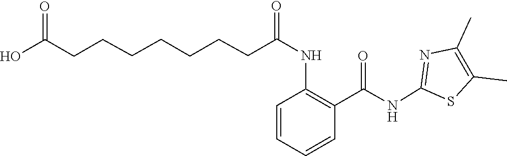 [SMILES: Cc1nc(NC(=O)c2ccccc2NC(=O)CCCCCCCC(=O)O)sc1C] | 9-((2-((4,5- dimethylthiazol-2- yl)carbamoyl)phenyl) amino)-9-oxononanoic acid |
| BL1- 156 | 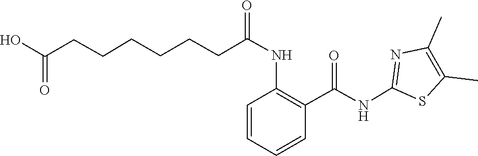 [SMILES: Cc1nc(NC(=O)c2ccccc2NC(=O)CCCCCCC(=O)O)sc1C] | 8-((2-((4,5- dimethylthiazol-2- yl)carbamoyl)phenyl) amino)-8-oxooctanoic acid |
| BL1- 157 | 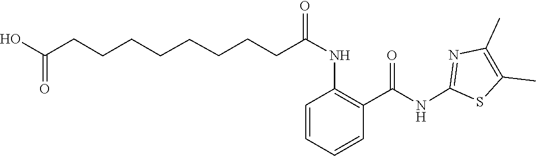 [SMILES: Cc1nc(NC(=O)c2ccccc2NC(=O)CCCCCCCCC(=O)O)sc1C] | 10-((2-((4,5- dimethylthiazol-2- yl)carbamoyl) phenyl)amino)- 10-oxodecanoic acid |
| BL1- 158 | 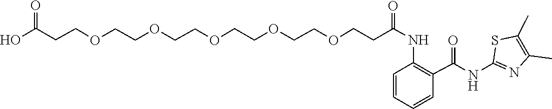 [SMILES: Cc1nc(NC(=O)c2ccccc2NC(=O)CCOCCOCCOCCOCCOCCC(=O)O)sc1C] | 19-((2-((4,5- dimethylthiazol-2- yl)carbamoyl)phenyl) amino)-19-oxo- 4,7,10,13,16- pentaoxanonadecanoic acid |
| BL1- 159 | 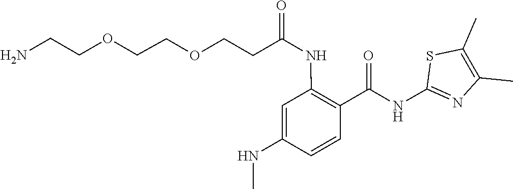 [SMILES: CNc1ccc(C(=O)Nc2nc(C)c(C)s2)c(NC(=O)CCOCCOCCN)c1] | 2-(3-(2-(2-aminoethoxy) ethoxy)propanamido)- N-(4,5-dimethylthiazol-2- yl)-4-(methylamino) benzamide |
| BL1- 160 | 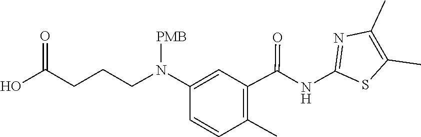 | 4-((3-((4,5- dimethylthiazol-2- yl)carbamoyl)-4- methylphenyl)(4- methoxybenzyl)amino) butanoic acid |
| BL1- 161 | 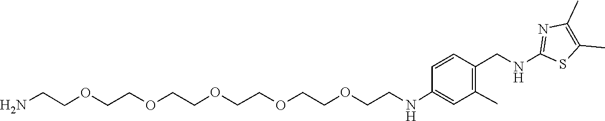 [SMILES: Cc1cc(NCCOCCOCCOCCOCCOCCN)ccc1CNc1nc(C)c(C)s1] | N¹-(4-(((4,5- dimethylthiazol-2- yl)amino)methyl)-3- methylphenyl)-3,6,9,12,15- pentaoxaheptadecane-1,17- diamine |
| BL1- 162 | 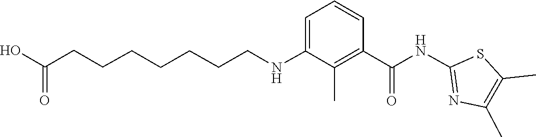 [SMILES: Cc1nc(NC(=O)c2cccc(NCCCCCCCC(=O)O)c2C)sc1C] | 8-((3-((4,5-dimethylthiazol- 2-yl)carbamoyl)-2- methylphenyl)amino) octanoic acid |
| BL1- 163 | 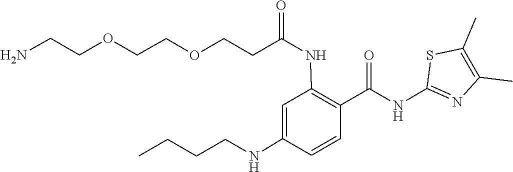 [SMILES: CCCCNc1ccc(C(=O)Nc2nc(C)c(C)s2)c(NC(=O)CCOCCOCCN)c1] | 2-(3-(2-(2-aminoethoxy) ethoxy)propanamido)- 4-(butylamino)-N-(4,5- dimethylthiazol-2-yl) benzamide |
| BL1- 164 | 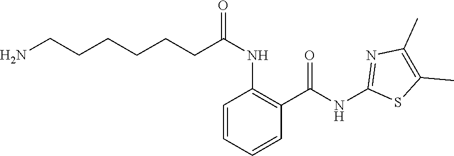 [SMILES: Cc1nc(NC(=O)c2ccccc2NC(=O)CCCCCCN)sc1C] | 2-(7-aminoheptanamido)-N- (4,5-dimethylthiazol-2- yl)benzamide |
| BL1- 165 | 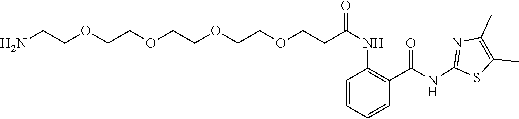 [SMILES: Cc1nc(NC(=O)c2ccccc2NC(=O)CCOCCOCCOCCOCCN)sc1C] | 1-amino-N-(2-((4,5- dimethylthiazol-2- yl)carbamoyl)phenyl)- 3,6,9,12-tetraoxapentadecan- 15-amide |
| BL1- 166 | 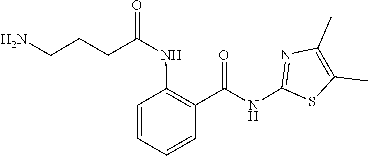 [SMILES: Cc1nc(NC(=O)c2ccccc2NC(=O)CCCN)sc1C] | 2-(4-aminobutanamido)- N-(4,5-dimethylthiazol- 2-yl)benzamide |
| BL1- 167 | 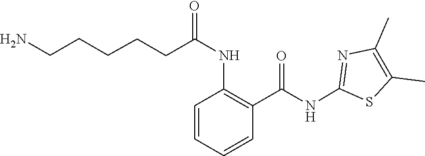 [SMILES: Cc1nc(NC(=O)c2ccccc2NC(=O)CCCCCN)sc1C] | 2-(6-aminohexanamido)-N- (4,5-dimethylthiazol-2- yl)benzamide |
| BL1- 168 | 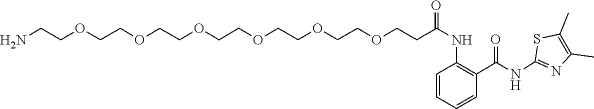 [SMILES: Cc1nc(NC(=O)c2ccccc2NC(=O)CCOCCOCCOCCOCCOCCOCCN)sc1C] | 1-amino-N-(2-((4,5- dimethylthiazol-2- yl)carbamoyl)phenyl)- 3,6,9,12,15,18- hexaoxahenicosan-21-amide |
| BL1- 169 | 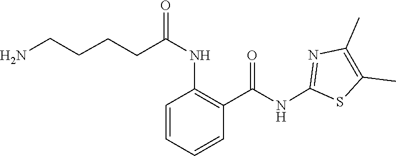 [SMILES: Cc1nc(NC(=O)c2ccccc2NC(=O)CCCCN)sc1C] | 2-(5-aminopentanamido)-N- (4,5-dimethylthiazol-2- yl)benzamide |
| BL1- 170 | 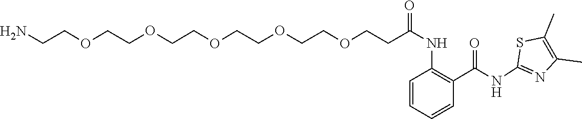 [SMILES: Cc1nc(NC(=O)c2ccccc2NC(=O)CCOCCOCCOCCOCCOCCN)sc1C] | 1-amino-N-(2-((4,5- dimethylthiazol-2- yl)carbamoyl)phenyl)- 3,6,9,12,15- pentaoxaoctadecan- 18-amide |
| BL1- 171 | 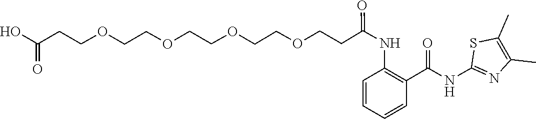 [SMILES: Cc1nc(NC(=O)c2ccccc2NC(=O)CCOCCOCCOCCOCCC(=O)O)sc1C] | 16-((2-((4,5- dimethylthiazol-2- yl)carbamoyl)phenyl)amino)- 16-oxo-4,7,10,13- tetraoxahexadecanoic acid |
| BL1- 172 | 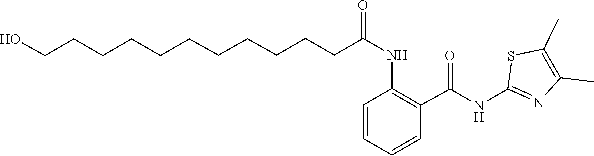 [SMILES: Cc1nc(NC(=O)c2ccccc2NC(=O)CCCCCCCCCCCO)sc1C] | N-(4,5-dimethylthiazol-2-yl)- 2-(12-hydroxy- dodecanamido)benzamide |
| BL1- 173 | 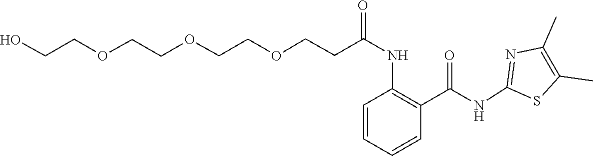 [SMILES: Cc1nc(NC(=O)c2ccccc2NC(=O)CCOCCOCCOCCO)sc1C] | N-(4,5-dimethylthiazol-2-yl)- 2-(3-(2-(2-(2- hydroxyethoxy) ethoxy)ethoxy) propanamido)benzamide |
| BL1- 174 | 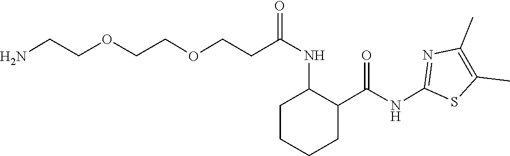 [SMILES: Cc1nc(NC(=O)C2CCCCC2NC(=O)CCOCCOCCN)sc1C] | 2-(3-(2-(2-aminoethoxy) ethoxy)propanamido)- N-(4,5-dimethylthiazol-2- yl)cyclohexane-1- carboxamide |
| BL1- 175 | 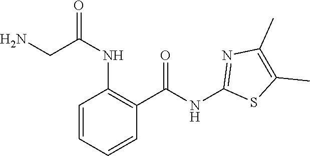 [SMILES: Cc1nc(NC(=O)c2ccccc2NC(=O)CN)sc1C] | 2-(2-aminoacetamido)- N-(4,5-dimethylthiazol- 2-yl)benzamide |
| BL1- 176 | 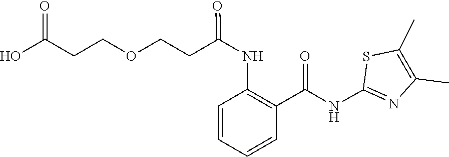 [SMILES: Cc1nc(NC(=O)c2ccccc2NC(=O)CCOCCC(=O)O)sc1C] | 3-(3-((2-((4,5- dimethylthiazol-2-yl) carbamoyl)phenyl)amino)- 3-oxopropoxy)propanoic acid |
| BL1- 177 | 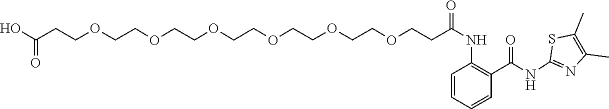 [SMILES: Cc1nc(NC(=O)c2ccccc2NC(=O)CCOCCOCCOCCOCCOCCOCCC(=O)O)sc1C] | 22-((2-((4,5- dimethylthiazol-2-yl) carbamoyl)phenyl)amino)- 22-oxo-4,7,10,13,16,19- hexaoxadocosanoic acid |
| BL1- 178 | 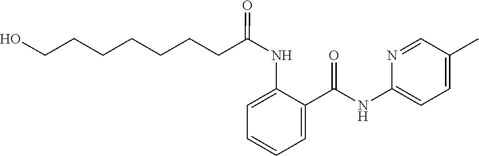 [SMILES: Cc1ccc(NC(=O)c2ccccc2NC(=O)CCCCCCCO)nc1] | 2-(8-hydroxyoctanamido)- N-(5-methylpyridin- 2-yl)benzamide |
| BL1- 179 | 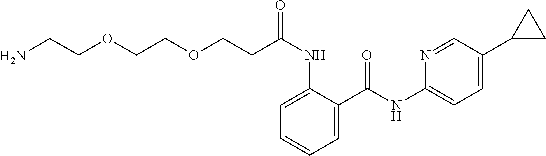 [SMILES: NCCOCCOCCC(=O)Nc1ccccc1C(=O)Nc1ccc(C2CC2)cn1] | 2-(3-(2-(2-aminoethoxy) ethoxy)propanamido)- N-(5-cyclopropylpyridin-2- yl)benzamide |
| BL1- 180 | 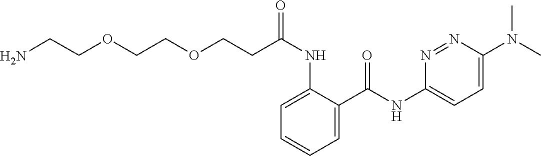 [SMILES: CN(C)c1ccc(NC(=O)c2ccccc2NC(=O)CCOCCOCCN)nn1] | 2-(3-(2-(2- aminoethoxy)ethoxy) propanamido)-N-(6- (dimethylamino)pyridazin-3- yl)benzamide |
| BL1- 181 | 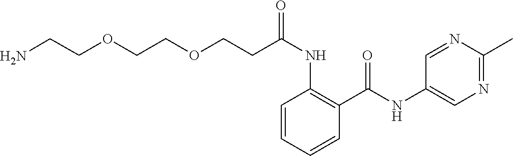 [SMILES: Cc1ncc(NC(=O)c2ccccc2NC(=O)CCOCCOCCN)cn1] | 2-(3-(2-(2-aminoethoxy) ethoxy)propanamido)- N-(2-methylpyrimidin-5- yl)benzamide |
| BL1- 182 | 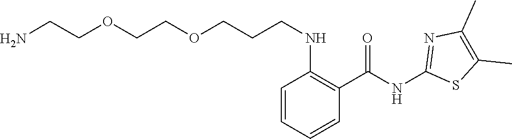 [SMILES: Cc1nc(NC(=O)c2ccccc2NCCCOCCOCCN)sc1C] | 2-((3-(2-(2- aminoethoxy)ethoxy)propyl) amino)-N-(4,5- dimethylthiazol-2- yl)benzamide |
| BL1- 183 | 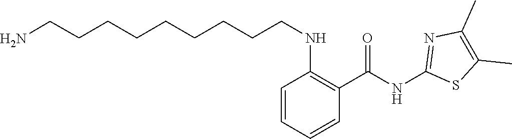 [SMILES: Cc1nc(NC(=O)c2ccccc2NCCCCCCCCCN)sc1C] | 2-((9-aminononyl)amino)-N- (4,5-dimethylthiazol-2- yl)benzamide |
| BL1- 184 | 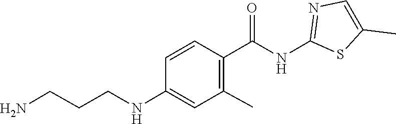 [SMILES: Cc1cnc(NC(=O)c2ccc(NCCCN)cc2C)s1] | 4-((3-aminopropyl)amino)-2- methyl-N-(5-methylthiazol-2- yl)benzamide |
| BL1- 185 | 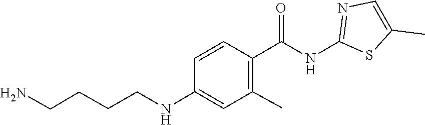 [SMILES: Cc1cnc(NC(=O)c2ccc(NCCCCN)cc2C)s1] | 4-((4-aminobutyl)amino)-2- methyl-N-(5-methylthiazol-2- yl)benzamide |
| BL1- 186 | 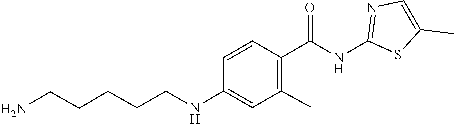 [SMILES: Cc1cnc(NC(=O)c2ccc(NCCCCCN)cc2C)s1] | 4-((5-aminopentyl)amino)-2- methyl-N-(5-methylthiazol-2- yl)benzamide |
| BL1- 187 | 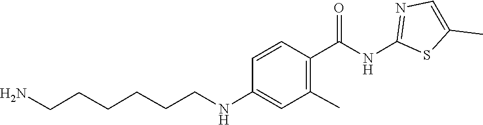 [SMILES: Cc1cnc(NC(=O)c2ccc(NCCCCCCN)cc2C)s1] | 4-((6-aminohexyl)amino)-2- methyl-N-(5-methylthiazol-2- yl)benzamide |
| BL1- 188 | 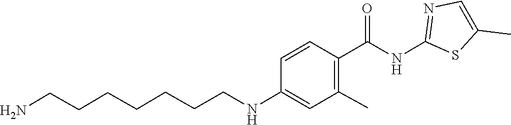 [SMILES: Cc1cnc(NC(=O)c2ccc(NCCCCCCCN)cc2C)s1] | 4-((7-aminoheptyl)amino)-2- methyl-N-(5-methylthiazol-2- yl)benzamide |
| BL1- 189 | 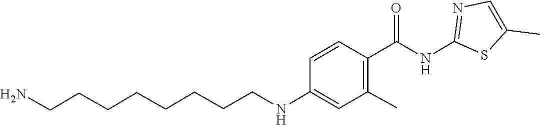 [SMILES: Cc1cnc(NC(=O)c2ccc(NCCCCCCCCN)cc2C)s1] | 4-((8-aminooctyl)amino)-2- methyl-N-(5-methylthiazol-2- yl)benzamide |
| BL1- 190 | 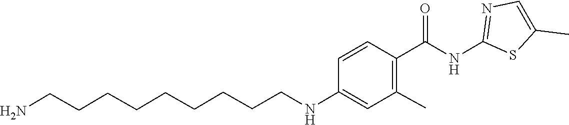 [SMILES: Cc1cnc(NC(=O)c2ccc(NCCCCCCCCCN)cc2C)s1] | 4-((9-aminononyl)amino)-2- methyl-N-(5-methylthiazol-2- yl)benzamide |
| BL1- 191 | 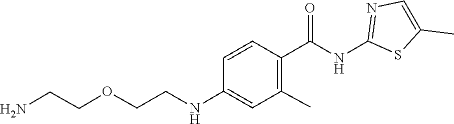 [SMILES: Cc1cnc(NC(=O)c2ccc(NCCOCCN)cc2C)s1] | 4-((2-(2- aminoethoxy)ethyl)amino)-2- methyl-N-(5-methylthiazol-2- yl)benzamide |
| BL1- 192 | 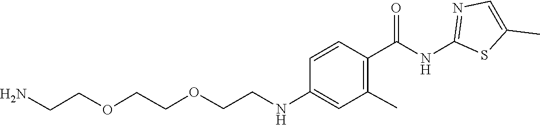 [SMILES: Cc1cnc(NC(=O)c2ccc(NCCOCCOCCN)cc2C)s1] | 4-((2-(2-(2- aminoethoxy)ethoxy)ethyl) amino)-2-methyl-N-(5- methylthiazol-2-yl) benzamide |
| BL1- 193 | 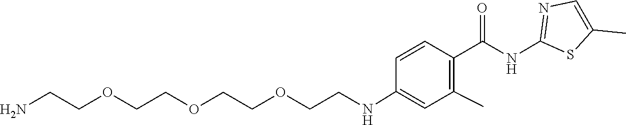 [SMILES: Cc1cnc(NC(=O)c2ccc(NCCOCCOCCOCCN)cc2C)s1] | 4-((2-(2-(2-(2- aminoethoxy)ethoxy)ethoxy) ethyl)amino)-2-methyl-N-(5- methylthiazol-2-yl) benzamide |
| BL1- 194 | 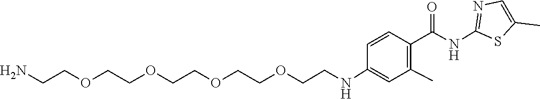 [SMILES: Cc1cnc(NC(=O)c2ccc(NCCOCCOCCOCCOCCN)cc2C)s1] | 4-((14-amino-3,6,9,12- tetraoxatetradecyl) amino)-2-methyl-N- (5-methylthiazol-2- yl)benzamide |
| BL1- 195 | 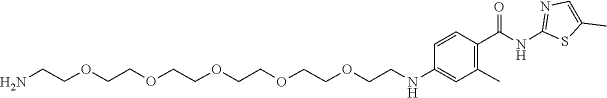 [SMILES: Cc1cnc(NC(=O)c2ccc(NCCOCCOCCOCCOCCOCCN)cc2C)s1] | 4-((17-amino-3,6,9,12,15- pentaoxaheptadecyl) amino)-2-methyl-N- (5-methylthiazol-2- yl)benzamide |
| BL1- 196 | 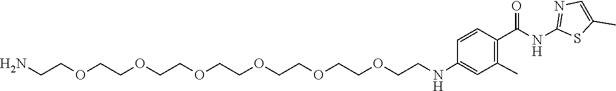 [SMILES: Cc1cnc(NC(=O)c2ccc(NCCOCCOCCOCCOCCOCCOCCN)cc2C)s1] | 4-((20-amino-3,6,9,12,15,18- hexaoxaicosyl)amino)-2- methyl-N-(5-methylthiazol- 2-yl)benzamide |
| BL1- 197 | 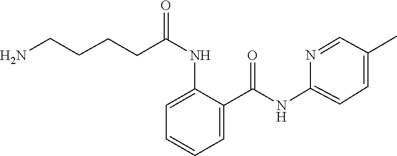 [SMILES: Cc1ccc(NC(=O)c2ccccc2NC(=O)CCCCN)nc1] | 2-(5-aminopentanamido)- N-(5-methylpyridin- 2-yl)benzamide |
| BL1- 198 | 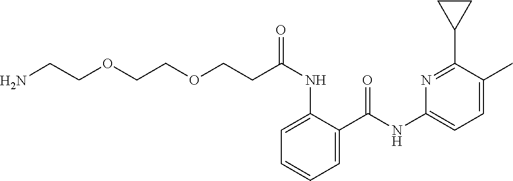 [SMILES: Cc1ccc(NC(=O)c2ccccc2NC(=O)CCOCCOCCN)nc1C1CC1] | 2-(3-(2-(2-aminoethoxy) ethoxy)propanamido)- N-(6-cyclopropyl-5- methylpyridin-2-yl) benzamide |
| BL1- 199 | 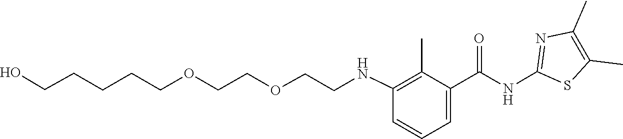 [SMILES: Cc1nc(NC(=O)c2cccc(NCCOCCOCCCCCO)c2C)sc1C] | N-(4,5-dimethylthiazol-2-yl)- 3-((2-(2-((5-hydroxypentyl) oxy)ethoxy)ethyl) amino)-2-methylbenzamide |
| BL1- 200 | 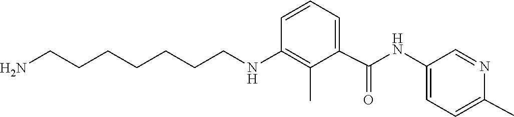 [SMILES: Cc1ccc(NC(=O)c2cccc(NCCCCCCCN)c2C)cn1] | 3-((7-aminoheptyl)amino)-2- methyl-N-(6-methylpyridin- 3-yl)benzamide |
| BL1- 201 | 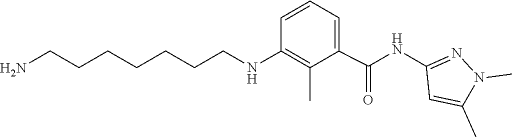 [SMILES: Cc1c(NCCCCCCCN)cccc1C(=O)Nc1cc(C)n(C)n1] | 3-((7-aminoheptyl)amino)-N- (1,5-dimethyl-1H-pyrazol-3- yl)-2-methylbenzamide |
| BL1- 202 | 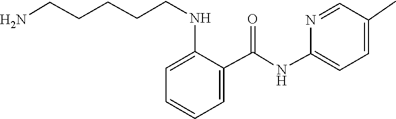 [SMILES: Cc1ccc(NC(=O)c2ccccc2NCCCCCN)nc1] | 2-((5-aminopentyl)amino)-N- (5-methylpyridin-2- yl)benzamide |
| BL1- 203 | 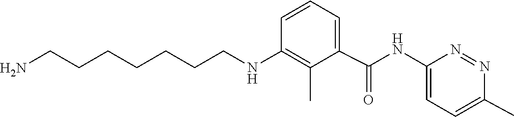 [SMILES: Cc1ccc(NC(=O)c2cccc(NCCCCCCCN)c2C)nn1] | 3-((7-aminoheptyl)amino)-2- methyl-N-(6-methylpyridazin- 3-yl)benzamide |
| BL1- 204 | 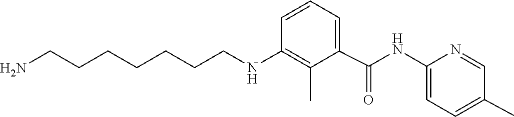 [SMILES: Cc1ccc(NC(=O)c2cccc(NCCCCCCCN)c2C)nc1] | 3-((7-aminoheptyl)amino)-2- methyl-N-(5-methylpyridin-2- yl)benzamide |
| BL1- 205 | 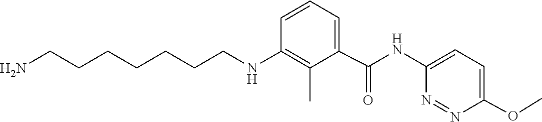 [SMILES: COc1ccc(NC(=O)c2cccc(NCCCCCCCN)c2C)nn1] | 3-((7-aminoheptyl)amino)-N- (6-methoxypyridazin-3-yl)-2- methylbenzamide |
| BL1- 206 | 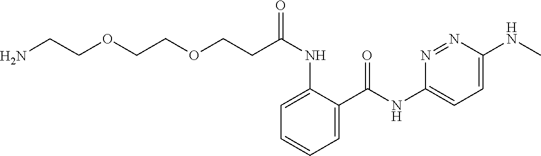 [SMILES: CNc1ccc(NC(=O)c2ccccc2NC(=O)CCOCCOCCN)nn1] | 2-(3-(2-(2- aminoethoxy)ethoxy) propanamido)-N-(6- (methylamino)pyridazin-3- yl)benzamide |
| BL1- 207 | 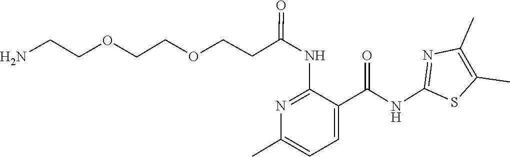 [SMILES: Cc1ccc(C(=O)Nc2nc(C)c(C)s2)c(NC(=O)CCOCCOCCN)n1] | 2-(3-(2-(2-aminoethoxy) ethoxy)propanamido)- N-(4,5-dimethylthiazol-2- yl)-6-methylnicotinamide |
| BL1- 208 | 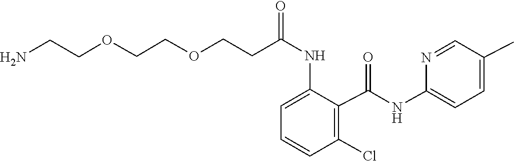 [SMILES: Cc1ccc(NC(=O)c2c(Cl)cccc2NC(=O)CCOCCOCCN)nc1] | 2-(3-(2-(2-aminoethoxy) ethoxy)propanamido)- 6-chloro-N-(5- methylpyridin-2-yl) benzamide |
| BL1- 209 | 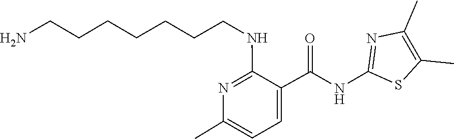 [SMILES: Cc1ccc(C(=O)Nc2nc(C)c(C)s2)c(NCCCCCCCN)n1] | 2-((7-aminoheptyl)amino)-N- (4,5-dimethylthiazol-2-yl)-6- methylnicotinamide |
| BL1- 210 | 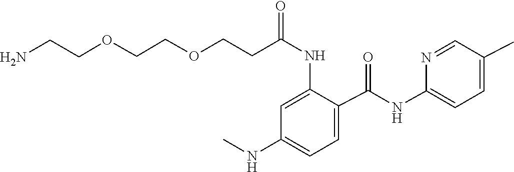 [SMILES: CNc1ccc(C(=O)Nc2ccc(C)cn2)c(NC(=O)CCOCCOCCN)c1] | 2-(3-(2-(2-aminoethoxy) ethoxy)propanamido)- 4-(methylamino)-N-(5- methylpyridin-2-yl) benzamide |
| BL1- 211 | 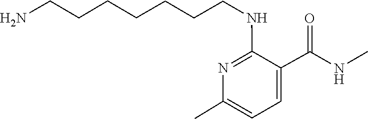 [SMILES: CNC(=O)c1ccc(C)nc1NCCCCCCCN] | 2-((7-aminoheptyl)amino)- N,6-dimethylnicotinamide |
| BL1- 212 | 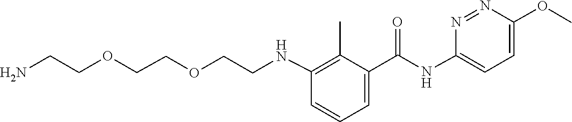 [SMILES: COc1ccc(NC(=O)c2cccc(NCCOCCOCCN)c2C)nn1] | 3-((2-(2-(2- aminoethoxy)ethoxy)ethyl) amino)-N-(6- methoxypyridazin- 3-yl)-2-methylbenzamide |
| BL1- 213 | 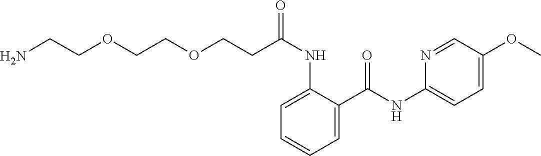 [SMILES: COc1ccc(NC(=O)c2ccccc2NC(=O)CCOCCOCCN)nc1] | 2-(3-(2-(2- aminoethoxy)ethoxy) propanamido)- N-(5-methoxypyridin-2- yl)benzamide |
| BL1- 214 | 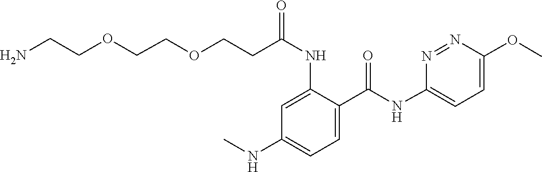 [SMILES: CNc1ccc(C(=O)Nc2ccc(OC)nn2)c(NC(=O)CCOCCOCCN)c1] | 2-(3-(2-(2- aminoethoxy)ethoxy) propanamido)- N-(6-methoxypyridazin-3- yl)-4-(methylamino) benzamide |
| BL1- 215 | 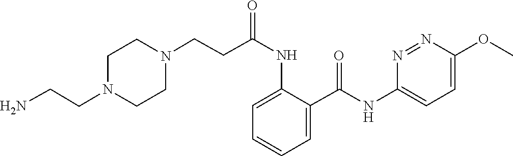 [SMILES: COc1ccc(NC(=O)c2ccccc2NC(=O)CCN2CCN(CCN)CC2)nn1] | 2-(3-(4-(2- aminoethyl)piperazin-1- yl)propanamido)-N-(6- methoxypyridazin-3- yl)benzamide |
| BL1- 216 | 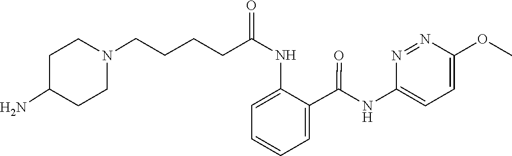 [SMILES: COc1ccc(NC(=O)c2ccccc2NC(=O)CCCCN2CCC(N)CC2)nn1] | 2-(5-(4-aminopiperidin-1- yl)pentanamido)-N-(6- methoxypyridazin-3- yl)benzamide |
| BL1- 217 | 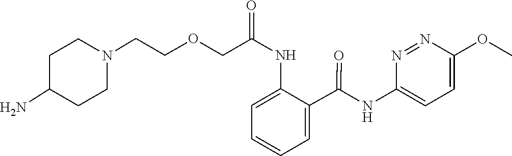 [SMILES: COc1ccc(NC(=O)c2ccccc2NC(=O)COCCN2CCC(N)CC2)nn1] | 2-(2-(2-(4-aminopiperidin-1- yl)ethoxy)acetamido)-N-(6- methoxypyridazin-3- yl)benzamide |
| BL1- 218 | 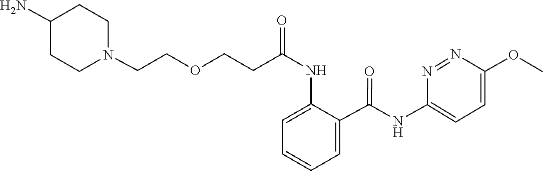 [SMILES: COc1ccc(NC(=O)c2ccccc2NC(=O)CCOCCN2CCC(N)CC2)nn1] | 2-(3-(2-(4-aminopiperidin-1- yl)ethoxy)propanamido)-N- (6-methoxypyridazin-3- yl)benzamide |
| BL1- 219 | 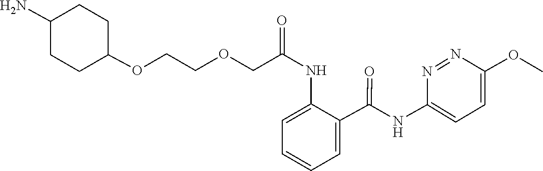 [SMILES: COc1ccc(NC(=O)c2ccccc2NC(=O)COCCOC2CCC(N)CC2)nn1] | 2-(2-(2-((4- aminocyclohexyl)oxy)ethoxy) acetamido)-N-(6- methoxypyridazin-3- yl)benzamide |
| BL1- 220 | 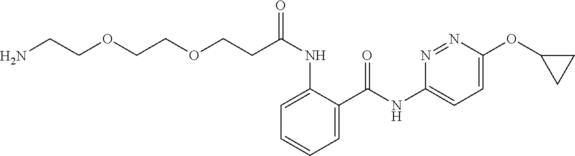 [SMILES: NCCOCCOCCC(=O)Nc1ccccc1C(=O)Nc1ccc(OC2CC2)nn1] | 2-(3-(2-(2- aminoethoxy)ethoxy) propanamido)-N-(6- cyclopropoxypyridazin-3- yl)benzamide |
| BL1- 221 | 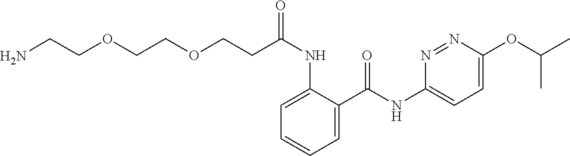 [SMILES: CC(C)Oc1ccc(NC(=O)c2ccccc2NC(=O)CCOCCOCCN)nn1] | 2-(3-(2-(2- aminoethoxy)ethoxy) propanamido)- N-(6-isopropoxypyridazin- 3-yl)benzamide |
| BL1- 222 | 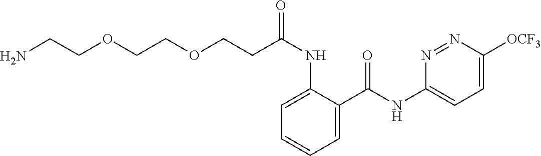 [SMILES: NCCOCCOCCC(=O)Nc1ccccc1C(=O)Nc1ccc(OC(F)(F)F)nn1] | 2-(3-(2-(2- aminoethoxy)ethoxy) propanamido)-N-(6- (trifluoromethoxy)pyridazin- 3-yl)benzamide |
| BL1- 223 | 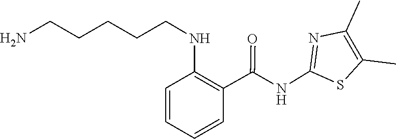 [SMILES: Cc1nc(NC(=O)c2ccccc2NCCCCCN)sc1C] | 2-((5-aminopentyl)amino)-N- (4,5-dimethylthiazol-2- yl)benzamide |
| BL1- 224 | 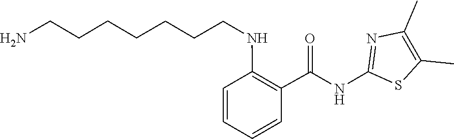 [SMILES: Cc1nc(NC(=O)c2ccccc2NCCCCCCCN)sc1C] | 2-((7-aminoheptyl)amino)-N- (4,5-dimethylthiazol-2- yl)benzamide |
| BL1- 225 | 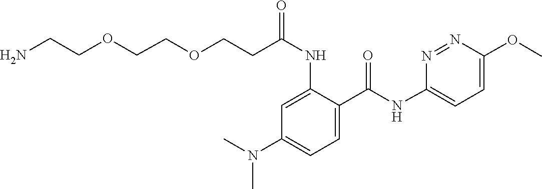 [SMILES: COc1ccc(NC(=O)c2ccc(N(C)C)cc2NC(=O)CCOCCOCCN)nn1] | 2-(3-(2-(2- aminoethoxy)ethoxy) propanamido)- 4-(dimethylamino)-N-(6- methoxypyridazin-3- yl)benzamide |
| BL1- 226 | 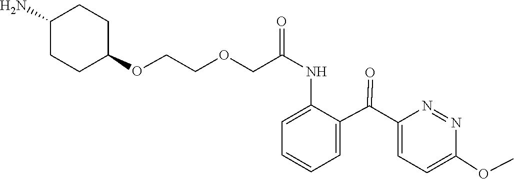 [SMILES: COc1ccc(C(=O)c2ccccc2NC(=O)COCCO[C@H]2CC[C@H](N)CC2)nn1] | 2-(2-((trans-4- aminocyclohexyl) oxy)ethoxy)-N-(2-(6- methoxypyridazine-3- carbonyl)phenyl)acetamide |
| BL1- 227 | 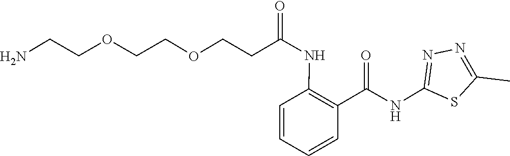 [SMILES: Cc1nnc(NC(=O)c2ccccc2NC(=O)CCOCCOCCN)s1] | 2-(3-(2-(2- aminoethoxy)ethoxy) propanamido)- N-(5-methyl-1,3,4- thiadiazol-2-yl)benzamide |
| BL1- 228 | 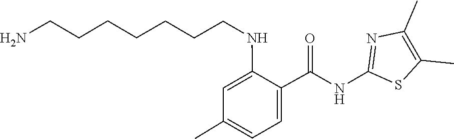 [SMILES: Cc1ccc(C(=O)Nc2nc(C)c(C)s2)c(NCCCCCCCN)c1] | 2-((7-aminoheptyl)amino)-N- (4,5-dimethylthiazol-2-yl)-4- methylbenzamide |
| BL1- 229 | 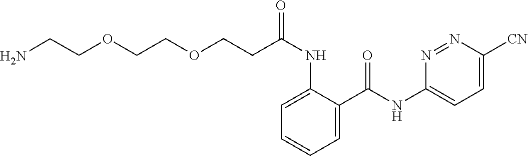 [SMILES: N#Cc1ccc(NC(=O)c2ccccc2NC(=O)CCOCCOCCN)nn1] | 2-(3-(2-(2- aminoethoxy)ethoxy) propanamido)- N-(6-cyanopyridazin-3- yl)benzamide |
| BL1- 230 | 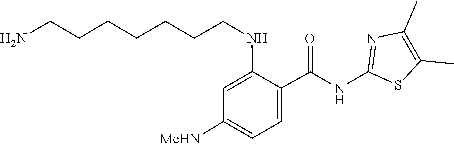 [SMILES: CNc1ccc(C(=O)Nc2nc(C)c(C)s2)c(NCCCCCCCN)c1] | 2-((7-aminoheptyl)amino)-N- (4,5-dimethylthiazol-2-yl)-4- (methylamino)benzamide |
| BL1- 231 | 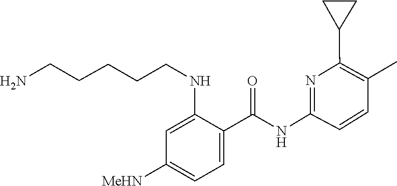 [SMILES: CNc1ccc(C(=O)Nc2ccc(C)c(C3CC3)n2)c(NCCCCCN)c1] | 2-((5-aminopentyl)amino)-N- (6-cyclopropyl-5- methylpyridin-2-yl)-4- (methylamino)benzamide |
| BL1- 232 | 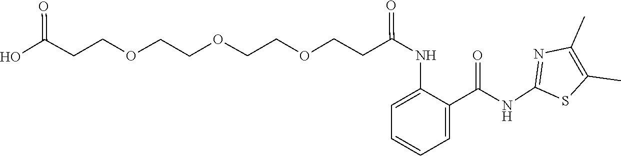 [SMILES: Cc1nc(NC(=O)c2ccccc2NC(=O)CCOCCOCCOCCC(=O)O)sc1C] | 3-(2-(2-(3-((2-((4,5- dimethylthiazol-2- yl)carbamoyl)phenyl) amino)-3-oxopropoxy) ethoxy)ethoxy) propanoic acid |
| BL1- 233 | 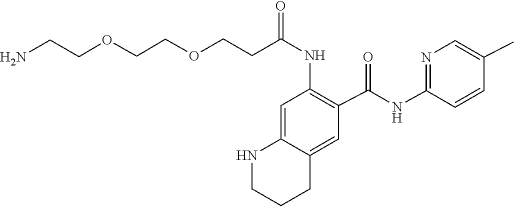 [SMILES: Cc1ccc(NC(=O)c2cc3c(cc2NC(=O)CCOCCOCCN)NCCC3)nc1] | 7-(3-(2-(2- aminoethoxy)ethoxy) propanamido)- N-(5-methylpyridin-2-yl)- 1,2,3,4-tetrahydroquinoline- 6-carboxamide |
| BL1- 234 | 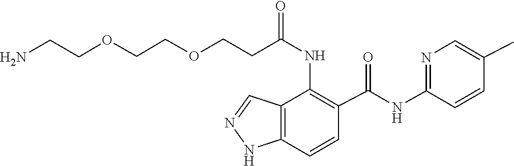 [SMILES: Cc1ccc(NC(=O)c2ccc3[nH]ncc3c2NC(=O)CCOCCOCCN)nc1] | 4-(3-(2-(2- aminoethoxy)ethoxy) propanamido)- N-(5-methylpyridin-2-yl)- 1H-indazole-5-carboxamide |
| BL1- 235 | 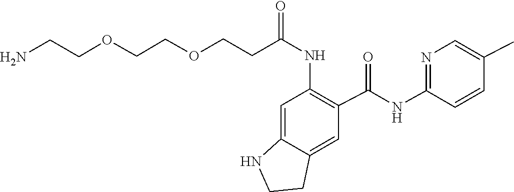 [SMILES: Cc1ccc(NC(=O)c2cc3c(cc2NC(=O)CCOCCOCCN)NCC3)nc1] | 6-(3-(2-(2- aminoethoxy)ethoxy) propanamido)-N-(5- methylpyridin-2- yl)indoline-5-carboxamide |
| BL1- 236 | 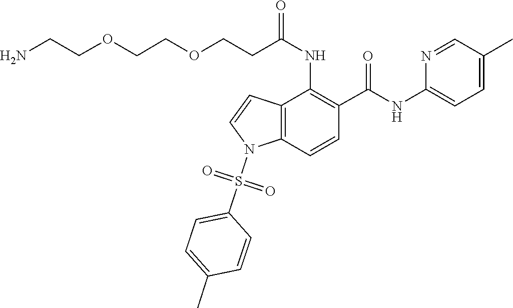 [SMILES: Cc1ccc(S(=O)(=O)n2ccc3c(NC(=O)CCOCCOCCN)c(C(=O)Nc4ccc(C)cn4)ccc32)cc1] | 4-(3-(2-(2- aminoethoxy)ethoxy)propan- amido)-N-(5-methylpyridin- 2-yl)-1-tosyl-1H-indole-5- carboxamide |
| BL1- 237 | 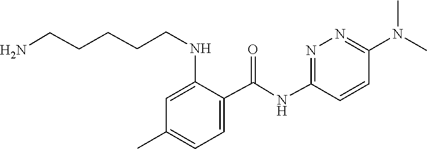 [SMILES: Cc1ccc(C(=O)Nc2ccc(N(C)C)nn2)c(NCCCCCN)c1] | 2-((5-aminopentyl)amino)- N-(6-(dimethylamino) pyridazin-3- yl)-4-methylbenzamide |
| BL1- 238 | 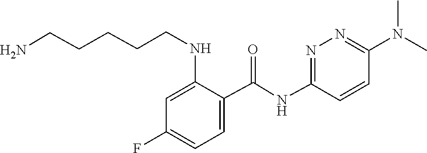 [SMILES: CN(C)c1ccc(NC(=O)c2ccc(F)cc2NCCCCCN)nn1] | 2-((5-aminopentyl)amino)- N-(6-(dimethylamino) pyridazin-3- yl)-4-fluorobenzamide |
| BL1- 239 | 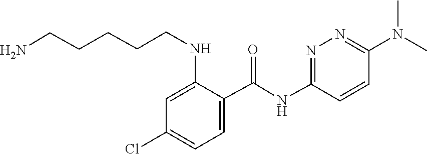 [SMILES: CN(C)c1ccc(NC(=O)c2ccc(Cl)cc2NCCCCCN)nn1] | 2-((5-aminopentyl)amino)-4- chloro-N-(6- (dimethylamino)pyridazin-3- yl)benzamide |
| BL1- 240 | 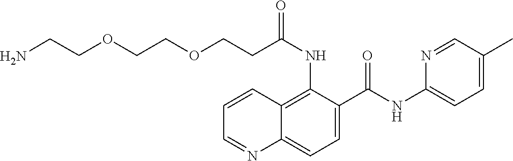 [SMILES: Cc1ccc(NC(=O)c2ccc3ncccc3c2NC(=O)CCOCCOCCN)nc1] | 5-(3-(2-(2- aminoethoxy)ethoxy) propanamido)-N-(5- methylpyridin-2- yl)quinoline-6-carboxamide |
| BL1- 241 | 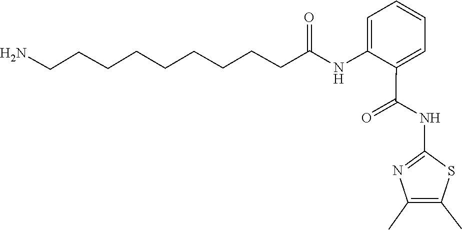 [SMILES: Cc1nc(NC(=O)c2ccccc2NC(=O)CCCCCCCCCN)sc1C] | 2-(10-aminodecanamido)-N- (4,5-dimethylthiazol-2- yl)benzamide |

### Target Protein Binding Moieties

Disclosed herein, in some embodiments, are compounds comprising a target protein binding moiety. The compound may comprise a heterobifunctional molecule comprising the target protein binding moiety.

Disclosed herein, in some embodiments, are target proteins. In some embodiments, a target protein comprises a kinase. In some embodiments, a target protein comprises a cyclin-dependent kinase. In some embodiments, a target protein comprises a cyclin-dependent kinase (CDK). In some embodiments, a target protein comprises cyclin-dependent kinase 4 (CDK4) or cyclin-dependent kinase 6 (CDK6). In some embodiments, a target protein comprises CDK4. In some embodiments, a target protein comprises CDK6. In some embodiments, a target protein comprises CDK9. In some embodiments, a target protein comprises CDK, CDK1, CDK2, CDK3, CDK4, CDK6, CDK7, CDK8, CDK9, CDK10, CDK11, CDK12, or CDK13.

In some embodiments, A is a target protein binding moiety comprising a cyclin-dependent kinase 4 (CDK4) binding moiety or a cyclin-dependent kinase 6 (CDK6) binding moiety.

In some embodiments, A is a target protein binding moiety comprising a CBP and/or p300 binding moiety or a BRD4 binding moiety. In some embodiments, A is a target protein binding moiety comprising a CBP and/or p300 binding moiety. In some embodiments, A is a target protein binding moiety comprising a BRD4 binding moiety.

In some embodiments, A is a target protein binding moiety having the structure of Formula (A), or a pharmaceutically acceptable salt or solvate thereof:

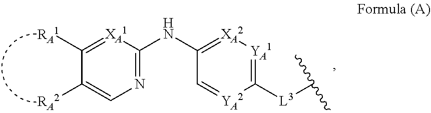

wherein,
X_{A}¹, X_{A}², Y_{A}¹, and Y_{A}² are each independently CR_{A}⁴ or N;
R_{A}¹ is NR_{A}⁵R_{A}⁶, N(R_{A}⁵)C(═O)R_{A}⁶, aryl, or heteroaryl;
R_{A}² is hydrogen, halogen, CN, NO₂, C₁-C₈ alkyl, C₁-C₈ haloalkyl, C₁-C₈ alkoxy, C₁-C₈ heteroalkyl, C₃-C₈ cycloalkyl, or C₂-C₈ heterocyclyl, or
R_{A}¹ and R_{A}², together with the atom(s) to which they are attached optionally form an optionally substituted carbocyclyl, heterocyclyl, aryl or heteroaryl;
L³ is a divalent group selected from —R_{A}^{3A}—R_{A}^{3B}—, wherein R_{A}^{3A} and R_{A}^{3B} are each independently a bond, —O—, —S—, —NR_{A}⁷—, —C(═O)—, —C(═O)NR_{A}⁷—, —S(═O)—, —S(═O)NR_{A}⁷—, —S(═O)₂—, —S(═O)₂NR_{A}⁷—, C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₃ cycloalkylene, C₂-C₁₂ heterocyclene, arylene, or heteroarylene;
each R_{A}⁴ is independently selected from hydrogen, halogen, CN, NO₂, NR_{A}⁸R_{A}⁹, —C(═O)R_{A}¹⁰, —C(═O)OR_{A}¹⁰, —C(═O)NR_{A}⁸R_{A}⁹, —NR_{A}⁸C(═O)R_{A}¹⁰, C₁-C₈ alkyl, C₁-C₈ haloalkyl, C₁-C₈ alkoxy, C₁-C₈ alkoxyalkyl, C₁-C₈ heteroalkyl, C₃-C₈ cycloalkyl, C₂-C₈ heterocyclyl, aryl, or heteroaryl; R_{A}⁵ and R_{A}⁶ are independently selected from hydrogen, C₁-C₈ alkyl, C₁-C₈ haloalkyl, C₁-C₈ alkoxyalkyl, C₁-C₈ heteroalkyl, C₃-C₈ cycloalkyl, C₂-C₈ heterocyclyl, aryl, or heteroaryl, or R_{A}⁵ and R_{A}⁶ together with the atom(s) to which they are connected optionally form a 3-20 membered heterocyclyl ring; and
R_{A}⁷, R_{A}⁸, R_{A}⁹ and R_{A}¹⁰ are each independently selected from hydrogen, C₁-C₈ alkyl, C₁-C₈ haloalkyl, C₁-C₈ alkoxyalkyl, C₁-C₈ heteroalkyl, C₃-C₈ cycloalkyl, C₂-C₈ hetero, aryl, or heteroaryl, or
R_{A}⁸ and R_{A}⁹ together with the atom(s) to which they are connected optionally form a 3-20 membered heterocyclyl ring.

In some embodiments, R_{A}¹ and R_{A}² together with the atom(s) to which they are connected, form an optionally substituted heterocyclyl or heteroaryl.

In some embodiments, the target protein binding moiety of Formula (A) has the structure of Formula (A1), (A2), or (A3), or a pharmaceutically acceptable salt or solvate thereof:

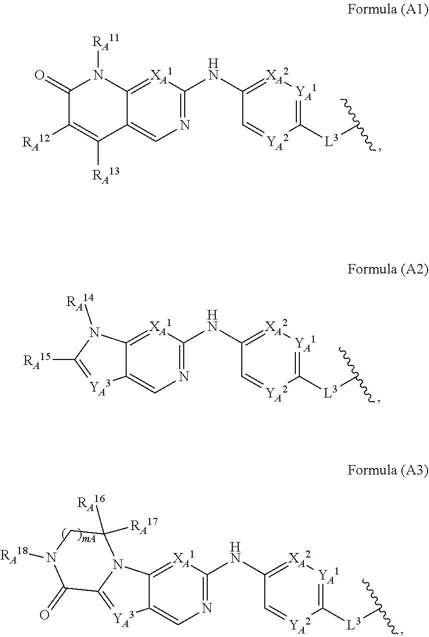

wherein
Y_{A}³ is CR_{A}⁹ or N;
R_{A}¹¹, R_{A}¹⁴ and R_{A}¹⁸ are each independently selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, or C₂-C₈ heterocyclyl, aryl, or heteroaryl;
R_{A}¹² and R_{A}¹⁵ are each independently selected from R_{A}²⁰, COR_{A}²⁰, CO₂R_{A}²⁰, or CONR_{A}²⁰R_{A}²¹, wherein R_{A}²⁰ and R_{A}²¹ are independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or C₂-C₈ heterocyclyl, or R_{A}²⁰ and R_{A}²¹, together with the atom(s) to which they are connected optionally form a 3-20 membered heterocyclyl ring;
R_{A}¹³ is selected from hydrogen, halogen, C₁-C₈ alkyl, C₁-C₈ haloalkyl, C₁-C₈ alkoxy, C₁-C₈ alkylamino, C₁-C₈ heteroalkyl, C₃-C₈ cycloalkyl, or C₂-C₈ heterocyclyl;
R_{A}¹⁶ and R_{A}¹⁷ are each independently selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, or C₂-C₈ heterocyclyl, aryl, or heteroaryl, or
R_{A}¹⁶ and R_{A}¹⁷, together with the atom(s) to which they are connected optionally form 3-8 membered cycloalkyl, or 3-8 membered heterocyclyl; and
R_{A}¹⁹ are independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or C₂-C₈ heterocyclyl; and
m_{A} is 0, 1, or 2.

In some embodiments, the target protein binding moiety of Formula (A) has the structure of Formula (A1), or a pharmaceutically acceptable salt or solvate thereof.

In some embodiments, the target protein binding moiety of Formula (A) has the structure of Formula (A2), or a pharmaceutically acceptable salt or solvate thereof.

In some embodiments, the target protein binding moiety of Formula (A) has the structure of Formula (A3), or a pharmaceutically acceptable salt or solvate thereof.

In some embodiments, m_{A} is 1.

In some embodiments, R_{A}¹ is aryl, or heteroaryl.

In some embodiments, the target protein binding moiety of Formula (A) has the structure of Formula (A4), or a pharmaceutically acceptable salt or solvate thereof:

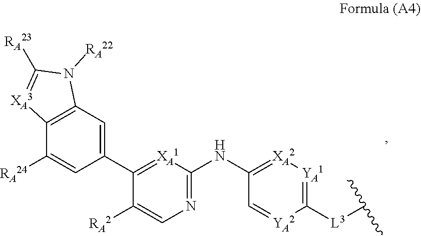

wherein
X_{A}³ is CR_{A}²⁵ or N;
R_{A}²² is selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, C₂-C₈ heterocyclyl, aryl, or heteroaryl; and
R_{A}²³, R_{A}²⁴ and R_{A}²⁵ are each independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or C₂-C₈ heterocyclyl.

In some embodiments, X_{A}¹, X_{A}², and X_{A}³ are each N. In some embodiments, X_{A}¹ is N. In some embodiments, X_{A}² is N. In some embodiments, X_{A3} is N.

In some embodiments, X_{A}¹ is CR_{A}⁴. In some embodiments, X_{A}² is CR_{A}⁴. In some embodiments, X_{A}³ is CR_{A}⁴. In some embodiments, X_{A}¹ is CH. In some embodiments, X_{A}² is CH. In some embodiments, X_{A}³ is CH.

In some embodiments, Y_{A1}, Y_{A}², and Y_{A}³ are each N. In some embodiments, Y_{A}¹ is N. In some embodiments, Y_{A}² is N. In some embodiments, Y_{A}³ is N

In some embodiments, Y_{A}¹ is CR_{A}⁴. In some embodiments, Y_{A}² is CR_{A}⁴. In some embodiments, Y_{A}³ is CR_{A}⁴. In some embodiments, Y_{A}¹, Y_{A}², and Y_{A}³ are each CH.

In some embodiments, R_{A}², R_{A}⁴, R_{A}¹³, R_{A}¹⁹, R_{A}²³, and R_{A}²⁴ are each independently selected from hydrogen, halogen, C₁-C₃ alkyl, or C₃-C₆ cycloalkyl. In some embodiments, R_{A}², R_{A}⁴, R_{A}¹³, R_{A}¹⁹, R_{A}²³, and R_{A}²⁴ are each independently selected from hydrogen, F, Cl, CH₃, CH₂CH₃, CH(CH₃)₂, CF₃, CHF₂, cyclopropyl, or cyclobutyl.

In some embodiments, R_{A}¹¹ and R_{A}¹⁴ are each independently selected from hydrogen, C₁-C₈ alkyl, C₃-C₈ cycloalkyl, or C₂-C₈ heterocyclyl. In some embodiments, R_{A}¹¹ and R_{A}¹⁴ are each independently selected from C₁-C₈ alkyl, or C₃-C₈ cycloalkyl. In some embodiments, R_{A}¹¹ and R_{A}¹⁴ are each independently selected from C₁-C₈ alkyl. In some embodiments, R_{A}¹¹ and R_{A}¹⁴ are each independently selected from C₃-C₈ cycloalkyl.

In some embodiments, R_{A}¹² and R_{A}¹⁵ are each independently selected from R_{A}²⁰, COR_{A}²⁰, or CONR_{A}²⁰R_{A}²¹, wherein R_{A}²⁰ and R_{A}²¹ are each independently selected from C₁-C₈ alkyl, C₃-C₈ cycloalkyl, or C₂-C₈ heterocyclyl. In some embodiments, R_{A}¹² and R_{A}¹⁵ are each independently selected from COR_{A}²⁰, or CONR_{A}²⁰R_{A}²¹, wherein R_{A}²⁰ and R_{A}²¹ are each independently selected from C₁-C₈ alkyl.

In some embodiments, R_{A}¹⁶ and R_{A}¹⁷ are each independently selected from hydrogen, C₁-C₈ alkyl, C₃-C₈ cycloalkyl, or C₂-C₈ heterocyclyl. In some embodiments, R_{A}¹⁶ and R_{A}¹⁷ are each independently selected from C₁-C₈ alkyl. In some embodiments, R_{A}¹⁶ and R_{A}¹⁷ are each independently selected from C₃-C₈ cycloalkyl. In some embodiments, R_{A}¹⁶ and R_{A}¹⁷ are each independently selected from C₂-C₈ heterocyclyl.

In some embodiments, R_{A}¹⁶ and R_{A}¹⁷ together with the atom(s) to which they are connected optionally form a 3-6 membered cycloalkyl or 3-6 membered heterocyclyl ring. In some embodiments, R_{A}¹⁶ and R_{A}¹⁷ together with the atom(s) to which they are connected optionally form a 3-6 membered cycloalkyl. In some embodiments, R_{A}¹⁶ and R_{A}¹⁷ together with the atom(s) to which they are connected optionally form a 3-6 membered heterocyclyl ring. In some embodiments, R_{A}¹⁸ and R_{A}²² are each independently selected from hydrogen, C₁-C₈ alkyl, C₃-C₈ cycloalkyl, or C₂-C₈ heterocyclyl. In some embodiments, R_{A}¹⁸ and R_{A}²² are each independently selected from H, CH₃, CH₂CH₃, CH(CH₃)₂, CF₃, CHF₂, cyclopropyl, or cyclobutyl.

In some embodiments, L³ is a divalent group selected from —R_{A}^{3A}—R_{A}^{3B}-, wherein R_{A}^{3A} and R_{A}^{3B} are each independently a bond, —O—, —S—, —NR_{A}⁷—, —C(═O)—, —C(═O)NR_{A}⁷—, —S(═O)—, —S(═O)NR_{A}⁷—, —S(═O)₂—, —S(═O)₂NR_{A}⁷—, C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₃ cycloalkylene, C₂-C₁₂ heterocyclene, arylene, or heteroarylene. In some embodiments, R_{A}^{3A} and R_{A}^{3B} are each independently a bond, —O—, —S—, —NR_{A}⁷—, —C(═O)—, —C(═O)NR_{A}⁷—, —S(═O)—, —S(═O)NR_{A}⁷—, —S(═O)₂—, —S(═O)₂NR_{A}⁷—. In some embodiments, R_{A}^{3A} and R_{A}^{3B} are each independently C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₃ cycloalkylene, C₃-C₁₃ heterocyclene, arylene, or heteroarylene.

In some embodiments, R_{A}^{3A} is selected from a bond, —O—, —S—, —NR_{A}⁷—, —C(═O)—, —C(═O)NR_{A}⁷—, —S(═O)—, —S(═O)NR_{A}⁷—, —S(═O)₂—, —S(═O)₂NR_{A}⁷—; and R_{A}^{3B} is selected from C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₃ cycloalkylene, C₃-C₁₃ heterocyclene, arylene, or heteroarylene. In some embodiments, R_{A}^{3B} is selected from a bond, —O—, —S—, —NR_{A}⁷—, —C(═O)—, —C(═O)NR_{A}⁷—, —S(═O)—, —S(═O)NR_{A}⁷—, —S(═O)₂—, —S(═O)₂NR_{A}⁷—; and R_{A}^{3A} is selected from C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₃ cycloalkylene, C₃-C₁₃ heterocyclene, aryl, or heteroarylene.

In some embodiments, L³ is a bond, C₁-C₃ alkylene, C₃-C₈ cycloalkylene, C₂-C₈ heteroalkylene, C₂-C₈ heterocyclene, —(C₁-C₃ alkylene)-(C₃-C₈ cycloalkylene)-, —(C₁-C₃ alkylene)-(C₂-C₈ heterocyclene)-, or —(C₁-C₃ alkylene)-(C₂-C₈ heteroalkylene).

In some embodiments, L³ is a bond. In some embodiments, L³ is C₁-C₃ alkylene. In some embodiments, L³ is C₃-C₈ cycloalkylene. In some embodiments, L³ is C₂-C₈ heteroalkylene. In some embodiments, L³ is C₂-C₈ heterocyclene. In some embodiments, L³ is —(C₁-C₃ alkylene)-(C₃-C₈ cycloalkylene)-. In some embodiments, L³ is —(C₁-C₃ alkylene)-(C₂-C₈ heterocyclene)-. In some embodiments, L³ is —(C₁-C₃ alkylene)-(C₂-C₈ heteroalkylene).

In some embodiments, L³ is a bond,

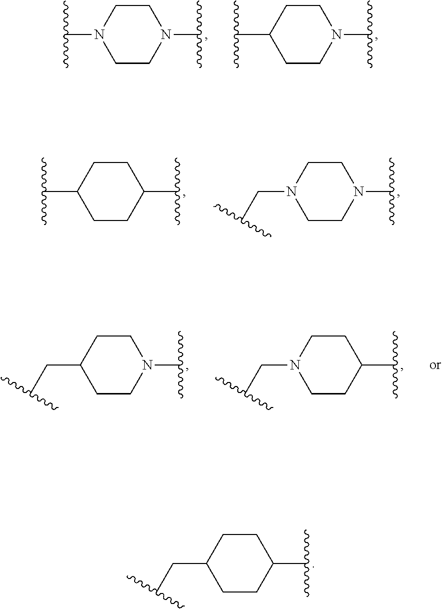

In some embodiments, L³ is

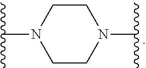 [SMILES: *N1CCN(*)CC1]

In some embodiments, L³ is

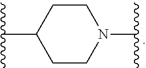 [SMILES: *C1CCN(*)CC1]

In some embodiments, L³ is

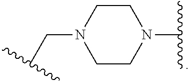 [SMILES: *CN1CCN(*)CC1]

In some embodiments, L³ is

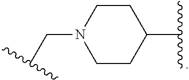 [SMILES: *CN1CCC(*)CC1]

In some embodiments, the target protein binding moiety of Formula (A) is selected from:

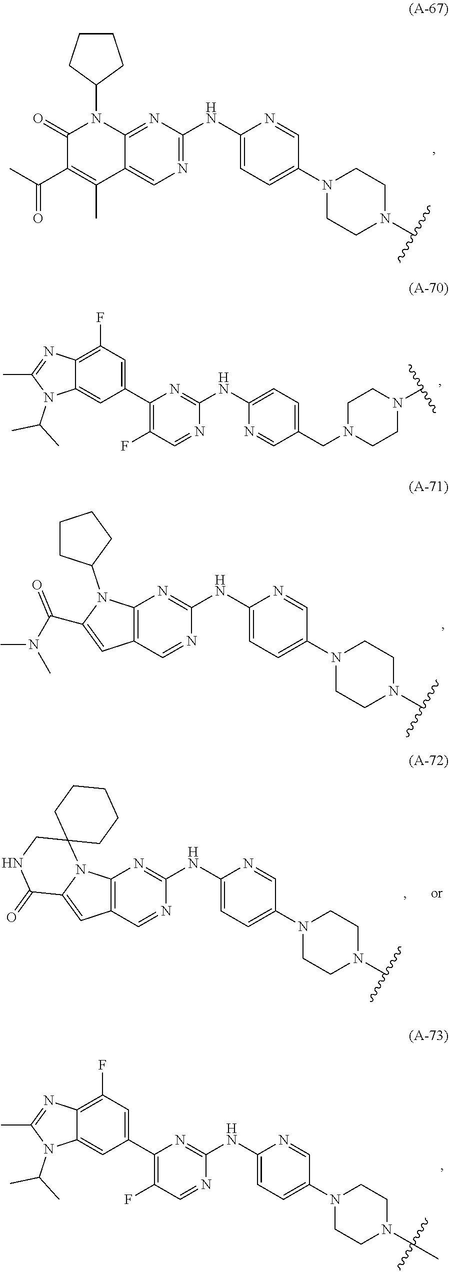 [SMILES: *N1CCN(c2ccc(Nc3ncc4c(C)c(C(C)=O)c(=O)n(C5CCCC5)c4n3)nc2)CC1 ; *N1CCN(Cc2ccc(Nc3ncc(F)c(-c4cc(F)c5nc(C)n(C(C)C)c5c4)n3)nc2)CC1 ; *N1CCN(c2ccc(Nc3ncc4cc(C(=O)N(C)C)n(C5CCCC5)c4n3)nc2)CC1 ; *N1CCN(c2ccc(Nc3ncc4cc5n(c4n3)C3(CCCCC3)CNC5=O)nc2)CC1 ; *N1CCN(c2ccc(Nc3ncc(F)c(-c4cc(F)c5nc(C)n(C(C)C)c5c4)n3)nc2)CC1]

or a pharmaceutically acceptable salt or solvate thereof.

In some embodiments, A is a target protein binding moiety having the structure of Formula (B-1), or a pharmaceutically acceptable salt or solvate thereof:

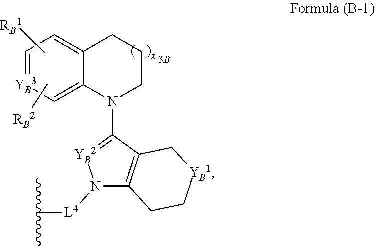

wherein,
Y_{B}¹ is CHR_{B}⁴ or NR_{B}⁴;
Y_{B}² is CH or N;
Y_{B}³ is CR_{B}² or N;
R_{B}¹ is a an optionally substituted 5-6 membered heteroaryl;
each R_{B}² is independently hydrogen, halogen, CN, NO₂, C₁-C₈ alkyl, C₁-C₈ haloalkyl, C₁-C₈ alkoxy, C₁-C₈ heteroalkyl, C₃-C₈ cycloalkyl, or C₂-C₈ heterocyclyl;
R_{B}⁴ is —C(═O)R_{B}⁸, —C(═O)OR_{B}⁸, —C(═O)NR_{B}⁶R_{B}⁷, or —NR_{B}⁶C(═O)R_{B}⁸;
L⁴ is a divalent group selected from —R_{B}^{3A}—R_{B}^{3B}—, wherein
R_{B}^{3A} and R_{B}^{3B} are each independently absent, a bond, —O—, —S—, —NR_{B}⁵—, —C(═O)—, —C(═O)NR_{B}⁵—, —S(═O)—, —S(═O)NR_{B}⁵—, —S(═O)₂—, —S(═O)₂NR_{B}⁵—, C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₃ cycloalkylene, C₂-C₁₂ heterocyclene, arylene, or heteroarylene;
R_{B}⁵, R_{B}⁶, R_{B}⁷ and R_{B}⁸ are each independently selected from C₁-C₈ alkyl, C₁-C₈ haloalkyl, C₁-C₈ alkoxyalkyl, C₁-C₈ heteroalkyl, C₃-C₈ cycloalkyl, C₂-C₈ heterocyclyl, aryl, or heteroaryl, or
R_{B}⁶ and R_{B}⁷ together with the atom(s) to which they are connected optionally form a 3-20 membered heterocyclyl ring; and
X_{3B} is 0, 1, or 2.

In some embodiments, Y_{B}² is CH. In some embodiments, Y_{B}² is N.

In some embodiments, x_{3B} is 1 or 2. In some embodiments, x_{3B} is 0. In some embodiments, x_{3B} is 1. In some embodiments, x_{3B} is 2.

In some embodiments, Y_{B}² is N; and X_{3B} is 1.

In some embodiments, Y_{B}¹ is C(R_{B}⁴)₂. In some embodiments, Y_{B}¹ is NR_{B}⁴.

In some embodiments, Y_{B}³ is CR_{B}². In some embodiments, Y_{B}² is N.

In some embodiments, A is a target protein binding moiety having the structure of Formula (B-2), or a pharmaceutically acceptable salt or solvate thereof:

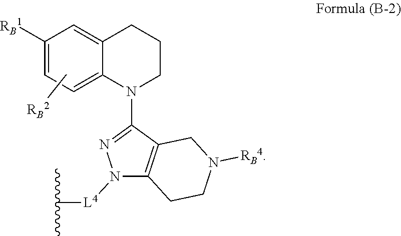

In some embodiments, R_{B}⁴ is —C(═O)R_{B}⁸ or —C(═O)OR_{B}⁸, or —C(═O)NR_{B}⁶R_{B}⁷.

In some embodiments, R_{B}⁴ is —C(═O)R_{B}⁸, wherein R_{B}⁸ is C₁-C₈ alkyl.

In some embodiments, R_{B}⁴ is —C(═O)NHR_{B}⁸ wherein R_{B}⁸ is C₁-C₈ alkyl.

In some embodiments, R_{B}² is halogen, CN, NO₂, C₁-C₈ alkyl, C₁-C₈ haloalkyl, or C₁-C₈ alkoxy. In some embodiments, R_{B}² is halogen, C₁-C₈ alkyl, or C₁-C₈ haloalkyl. In some embodiments, R_{B}² is Cl, F, Br, CH₃, CF₃, or CHF₂.

In some embodiments, R_{B}¹ is a an optionally substituted 5-membered heteroaryl selected from pyrrolyl, furanyl, imidazolyl, pyrazolyl, oxazolyl, isoxazolyl, thienyl, thiazolyl, isothiazolyl, triazolyl, oxadiazolyl, thiadiazolyl, or tetrazolyl. In some embodiments, R_{B}¹ is imidazolyl, pyrazolyl, oxazolyl, isoxazolyl, triazolyl, or tetrazolyl. In some embodiments, R_{B}¹ is an optionally substituted pyrazolyl. In some embodiments, R_{B}¹ is a methyl substituted pyrazolyl.

In some embodiments, L⁴ is a bond, C₁-C₃ alkylene, C₃-C₈ cycloalkylene, C₂-C₈ heteroalkylene, C₂-C₈ heterocyclene, —(C₁-C₃ alkylene)-(C₃-C₈ cycloalkylene)-, —(C₁-C₃ alkylene)-(C₂-C₈ heterocyclene)-, or —(C₁-C₃ alkylene)-(C₂-C₈ heteroalkylene)-.

In some embodiments, L⁴ is a bond,

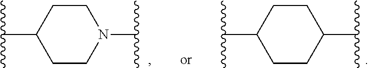 [SMILES: *C1CCN(*)CC1 ; *C1CCC(*)CC1]

In some embodiments, L⁴ is

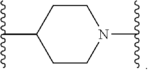 [SMILES: *C1CCN(*)CC1]

In some embodiments, L⁴ is a bond.

In some embodiments, the target protein binding moiety is:

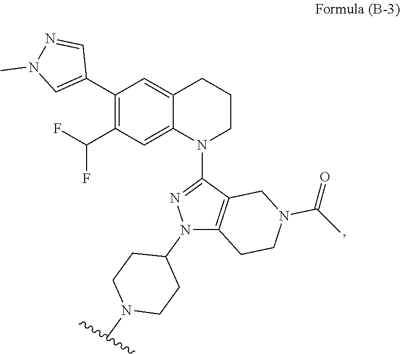 [SMILES: *N1CCC(n2nc(N3CCCc4cc(-c5cnn(C)c5)c(C(F)F)cc43)c3c2CCN(C(C)=O)C3)CC1]

or a pharmaceutically acceptable salt or solvate thereof.

In some embodiments, the target protein binding moiety is:

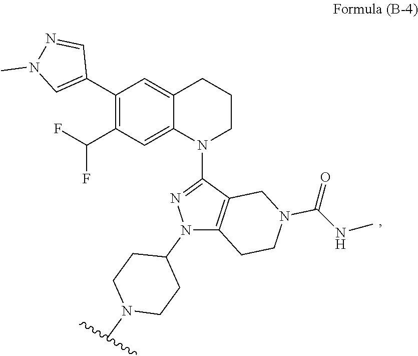

or a pharmaceutically acceptable salt or solvate thereof

In some embodiments, A is a target protein binding moiety having the structure of Formula (C-1), (C-2), (C-3), (C-4), (C-5), (C-6), or a pharmaceutically acceptable salt or solvate thereof:

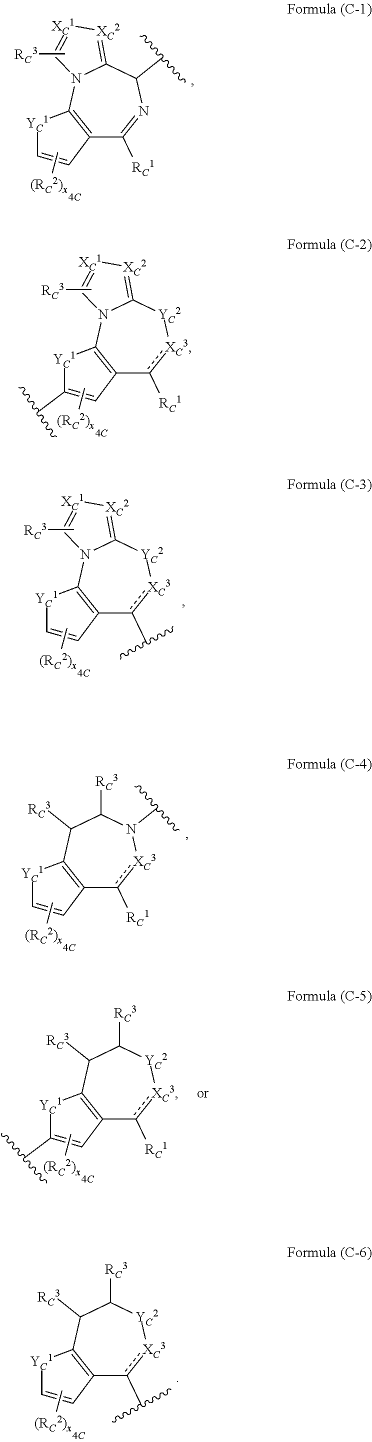

wherein,

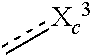

is

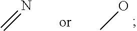

X_{C}¹ and X_{C}² are each independently CR_{C}³ or N;
Y_{C}¹ is O, S, or —C(R_{C}²)═C(R_{C}²)—;
Y_{C}² is C(R_{C}⁷)₂, or NR_{C}⁷;
R_{C}¹ is hydrogen or optionally substituted C₆-C₁₀ aryl or 5 to 10 membered heteroaryl;
each R_{C}² is independently hydrogen, halogen, CN, NO₂, NR_{C}⁴R_{C}⁵, —C(═O)R_{C}⁶, —C(═O)OR_{C}⁴, —C(═O)NR_{C}⁴R_{C}⁵, —OC(═O)R_{C}⁶, —N(R_{C}⁴)C(═O)R_{C}⁶, C₁-C₈ alkyl, C₁-C₈ heteroalkyl, C₂-C₈ alkynyl, C₁-C₈ haloalkyl, C₁-C₈ alkoxy, C₁-C₈ alkoxyalkyl, or C₁-C₈ alkylaryl;
each R_{C}³ is independently hydrogen, halogen, CN, NO₂, NR_{C}⁴R_{C}⁵, C₁-C₈ alkyl, C₁-C₈ haloalkyl, C₁-C₈ alkoxy, C₁-C₈ alkoxyalkyl, aryl, or heteroaryl;
R_{C}⁴, R_{C}⁵ and R_{C}⁶ are each independently selected from hydrogen, C₁-C₈ alkyl, C₁-C₈ haloalkyl, C₁-C₈ alkoxyalkyl, C₁-C₈ heteroalkyl, C₃-C₈ cycloalkyl, C₂-C₈ heterocyclyl, aryl, or heteroaryl, or
R_{C}⁴ and R_{C}⁵ together with the atom(s) to which they are connected optionally form a 3-20 membered heterocyclyl ring;
each R_{C}⁷ is independently hydrogen, NR_{C}⁴R_{C}⁵, OR_{C}⁴, —C(═O)R_{C}⁶, —C(═O)OR_{C}⁶, —C(═O)NR_{C}⁴R_{C}⁵, —(C₁-C₈ alkyl)—C(═O)NR_{C}⁴R_{C}⁵, —OC(═O)R_{C}⁶, —N(R_{C}⁸)C(═O)R_{C}⁶, C₁-C₈ alkyl, C₁-C₈ haloalkyl, C₁-C₈ heteroalkyl, C₃-C₈ cycloalkyl, C₂-C₈ heterocyclyl, or
two of R_{C}⁷, together with the atom(s) they are connected, optionally form a C₃-C₈ cycloalkyl, or C₂-C₈ heterocyclyl; and
x_{4C} is 1, 2, or 3.

In some embodiments,

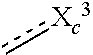

is

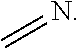

In some embodiments,

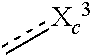

is

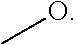

In some embodiments, X_{C}¹ and X_{C}² are each independently N. In some embodiments, X_{C}¹ and X_{C}² are each independently CR_{C}³. In some embodiments, X_{C}¹ is N and X_{C}² is CR_{C}³. In some embodiments, X_{C}² is N and X_{C}¹ is CR_{C}³.

In some embodiments, Y_{C}¹ is S. In some embodiments, Y_{C}¹ is O. In some embodiments, Y_{C}¹ is —C═C—. In some embodiments, Y_{C}¹ is —C(R_{C}²)═C(R_{C}²)—. In some embodiments, Y_{C}² is C(R_{C}⁷)₂, In some embodiments, Y_{C}² is NR_{C}⁷. In some embodiments, R_{C}³ is hydrogen, halogen, C₁-C₈ alkyl, C₁-C₈ haloalkyl, C₁-C₈ alkoxy, or C₁-C₈ alkoxyalkyl. In some embodiments, each R_{C}² is independently hydrogen, halogen, C₁-C₈ alkyl, C₂-C₈ alkynyl, C₁-C₈ haloalkyl, C₁-C₈ alkoxy, C₁-C₈ alkoxyalkyl, aryl, or heteroaryl. In some embodiments, R_{C}¹ is H. In some embodiments, R_{C}¹ is optionally substituted C₆-C₁₀ aryl, optionally substituted with 1-4 halogen, CN, NO₂, NR_{C}⁴R_{C}⁵, —C(═O)R_{C}⁶, —C(═O)OR_{C}⁶, —C(═O)NR_{C}⁴R_{C}⁵, C₁-C₈ alkyl, C₁-C₈ haloalkyl, C₁-C₈ alkoxy, or C₁-C₈ alkoxyalkyl. In some embodiments, x_{4C} is 2; and each R_{C}² is independently C₁-C₈ alkyl. In some embodiments, x_{4C} is 2; and each R_{C}² is independently C₁-C₈ alkoxy.

In some embodiments, each R_{C}² is independently halogen, C₁-C₈ alkyl, C₂-C₈ alkynyl, C₁-C₈ haloalkyl, C₁-C₈ alkoxy, C₁-C₈ alkoxyalkyl, aryl, or heteroaryl. In some embodiments, each R_{C}² is independently halogen, C₁-C₈ alkyl, C₁-C₈ haloalkyl, C₁-C₈ alkoxy, or C₁-C₈ alkoxyalkyl. In some embodiments, each R_{C}² is independently halogen. In some embodiments, each R_{C}² is independently CH₃, CH₂CH₃, CH(CH₃)₂, C(CH₃)₃, CH(CH₂)₂, CH₂Ph. In some embodiments, each R_{C}² is independently C₁-C₈ alkoxy. In some embodiments, each R_{C}² is independently OCH₃, OCH₂CH₃, OCH(CH₃)₂, OC(CH₃)₃, OCH(CH₂)₂. In some embodiments, each R_{C}² is independently C₂-C₈ alkynyl.

In some embodiments, each R_{C}² is independently —C≡C—, or

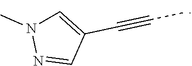

In some embodiments, each R_{C}² is independently heteroaryl. In some embodiments, each R_{C}² is independently 5-membered heteroaryl. In some embodiments, each R_{C}² is independently pyrrolyl, furanyl, imidazolyl, pyrazolyl, oxazolyl, isoxazolyl, thienyl, thiazolyl, isothiazolyl, triazolyl, oxadiazolyl, thiadiazolyl, or tetrazolyl. In some embodiments, each R_{C}² is independently 6-membered heteroaryl. In some embodiments, each R_{C}² is independently pyridinyl, pyridazinyl, pyrazinyl, pyrimidinyl, or triazinyl. In some embodiments, x₄ is 2; and each R_{C}² is independently C₁-C₈ alkyl. In some embodiments, x₄ is 2; and each R_{C}² is independently C₁-C₈ alkoxy. In some embodiments, each R_{C}² is independently C₁-C₈ alkyl. In some embodiments, each R_{C}² is independently CH₃, CH₂CH₃, CH(CH₃)₂, C(CH₃)₃.

In some embodiments, R_{C}³ is halogen, C₁-C₈ alkyl, C₁-C₈ haloalkyl, C₁-C₈ alkoxy, or C₁-C₈ alkoxyalkyl. In some embodiments, each R_{C}³ is independently halogen. In some embodiments, each R_{C}³ is independently C₁-C₈ alkyl. In some embodiments, each R_{C}³ is independently CH₃, CH₂CH₃, CH(CH₃)₂, C(CH₃)₃.

In some embodiments, R_{C}¹ is H. In some embodiments, R_{C}¹ is optionally substituted C₆-C₁₀ aryl, optionally substituted with 1-4 halogen, CN, NO₂, NR_{C}⁴R_{C}⁵, —C(═O)R_{C}⁶, —C(═O)OR_{C}⁶, —C(═O)NR_{C}⁴R_{C}⁵, C₁-C₈ alkyl, C₁-C₈ haloalkyl, C₁-C₈ alkoxy, or C₁-C₈ alkoxyalkyl. In some embodiments, R_{C}¹ is optionally substituted C₆ aryl, optionally substituted with 1-4 halogen, CN, NO₂, NR_{C}⁴R_{C}⁵, C₁-C₈ alkyl, C₁-C₈ haloalkyl, C₁-C₈ alkoxy, or C₁-C₈ alkoxyalkyl.

In some embodiments, R_{C}¹ is optionally substituted 5 to 10 membered heteroaryl optionally substituted with 1-4 halogen, CN, NO₂, NR_{C}⁴R_{C}⁵, C₁-C₈ alkyl, C₁-C₈ haloalkyl, C₁-C₈ alkoxy, or C₁-C₈ alkoxyalkyl.

In some embodiments, the target protein binding moiety is

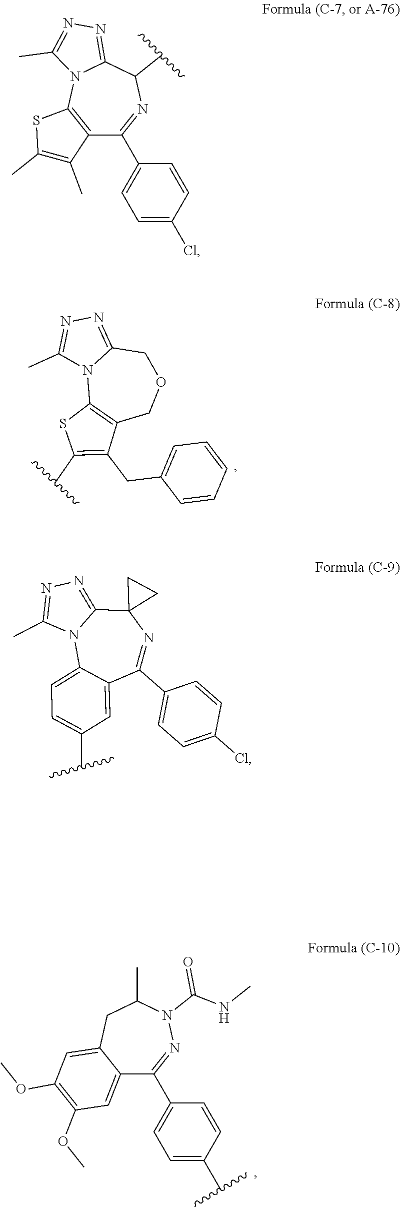

or a pharmaceutically acceptable salt or solvate thereof.

In some embodiments, the target protein binding moiety is

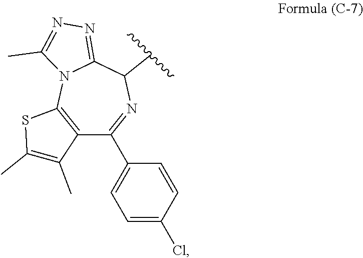 [SMILES: *C1N=C(c2ccc(Cl)cc2)c2c(sc(C)c2C)-n2c(C)nnc21]

or a pharmaceutically acceptable salt or solvate thereof.

In some embodiments, the target protein is described in WO2020173440A1, which is herein incorporated by reference in its entirety.

In some embodiments, the target protein comprises a cyclin D. In some embodiments, the target protein is cyclin D1. In some embodiments, the target protein is cyclin D2. In some embodiments, the target protein is cyclin D3.

In some embodiments, the target protein comprises a retinoblastoma (RB) protein. In some embodiments, the target protein is RB1. In some embodiments, the target protein is p107 (RBL1). In some embodiments, the target protein is p130 (RBL2).

Additional examples of target protein binding moieties may include haloalkane halogenase inhibitors, Hsp90 inhibitors, kinase inhibitors, MDM2 inhibitors, compounds targeting Human BET Bromodomain-containing proteins, HDAC inhibitors, human lysine methyltransferase inhibitors, angiogenesis inhibitors, immunosuppressive compounds, and compounds targeting the aryl hydrocarbon receptor (AHR). Some compounds include a small molecule target protein binding moiety. Such small molecule target protein binding moieties also include pharmaceutically acceptable salts, enantiomers, solvates and polymorphs of these compositions, as well as other small molecules that may target a protein of interest.

In some embodiments, the target protein binding moiety includes a heat shock protein (HSP; e.g. HSP90) binder or inhibitor. HSP90 inhibitors as used herein include, but are not limited to: N-[4-(3H-imidazo[4,5-C]pyridin-2-yl)-9H-fluoren-9-yl]-succinamide, 8-[(2,4-dimethylphenyl)sulfanyl]-3-pent-4-yn-1-yl-3H-purin-6-amine, 5-[2,4-dihydroxy-5-(1-methylethyl)phenyl]-N-ethyl-4-[4-(morpholin-4-ylmethyl)phenyl]isoxazole-3-carboxamide, PU3, or (4E,6Z,8S,9S,10E,12S,13R,14S,16R)-13-hydroxy-8,14,19-trimethoxy-4,10,12,16-tetramethyl-3,20,22-trioxo-2-azabicyclo[16.3.1] or any of its derivatives (e.g. 17-alkylamino-17-desmethoxygeldanamycin).

In some embodiments, N-[4-(3H-imidazo[4,5-C]pyridin-2-yl)-9H-fluoren-9-yl]-succinamide is attached via its terminal amide group to a linker described herein. In some embodiments, 8-[(2,4-dimethylphenyl)sulfanyl]-3-pent-4-yn-1-yl-3H-purin-6-amine is attached via its terminal acetylene group to a linker described herein. In some embodiments, 5-[2,4-dihydroxy-5-(1-methylethyl)phenyl]-N-ethyl-4-[4-(morpholin-4-ylmethyl)phenyl]isoxazole-3-carboxamide is attached via its amide group (e.g. at the amine or at the alkyl group on the amine) to a linker described herein. In some embodiments, PU3 is attached via its butyl group to a linker described herein. In some embodiments, (4E,6Z,8S,9S,10E,12S,13R,14S,16R)-13-hydroxy-8,14,19-trimethoxy-4,10,12,16-tetramethyl-3,20,22-trioxo-2-azabicyclo[16.3.1] or any of its derivatives are attached by an amide group to a linker described herein.

In some embodiments, the target protein binding moiety includes a kinase inhibitor or a phosphatase inhibitor. In some embodiments, the target protein binding moiety includes a kinase inhibitor. In some embodiments, the kinase inhibitor is a tyrosine kinase inhibitor. In some embodiments, the kinase inhibitor is a VEGFR3 inhibitor. In some embodiments, the kinase inhibitor is an aurora kinase inhibitor. In some embodiments, the kinase inhibitor is an ALK inhibitor. In some embodiments, the kinase inhibitor is a JAK2 inhibitor. In some embodiments, the kinase inhibitor is an Alk inhibitor. In some embodiments, the kinase inhibitor is a Met inhibitor. In some embodiments, the kinase inhibitor is an Abl inhibitor. In some embodiments, the kinase inhibitor is a B-Raf/Mek inhibitor.

Non-limiting examples of kinase inhibitors include any one of erlotinib, sunitinib, sorafenib, dasatinib, lapatinib, U09-CX-5279, Y1W, Y1X, 1-ethyl-3-(2-{[3-(1-methylethyl)[1,2,4]triazolo[4,3-a]pyridin-6-yl]sulfanyl}benzyl)urea, a 2,6-naphthyridine, 07U, YCF, XK9, NXP, N-{4-[(1E)-N—(N-hydroxycarbamimidoyl)ethanehydrazonoyl]phenyl}-7-nitro-1H-indole-2-carboxamide, afatinib, fostamatinib, gefitinib, lenvatinib, vandetanib, vemurafenib, gleevec, pazopanib, AT-9283, TAE684, nilotinib, NVP—BSK805, crizotinib, JNJ FMX, or foretinib.

In some embodiments, erlotinib is attached via its ether group to a linker described herein. In some embodiments, sunitinib is attached via its pyrrole moiety to a linker described herein. In some embodiments, sorafenib is attached via its phenyl moiety to a linker described herein. In some embodiments, dasatinib is attached via its pyrimidine to a linker described herein. In some embodiments, lapatinib is attached via its terminal methyl of its sulfonyl methyl group to a linker described herein. In some embodiments, U09-CX-5279 is attached via its amine (aniline), carboxylic acid or amine alpha to cyclopropyl group, or cyclopropyl group to a linker described herein. In some embodiments, 1-ethyl-3-(2-{[3-(1-methylethyl)[1,2,4]triazolo[4,3-a]pyridin-6-yl]sulfanyl}benzyl)urea is attached via its propyl group to a linker described herein. In some embodiments, Y1W is attached via its propyl or butyl group to a linker described herein. In some embodiments, 6TP is attached via a terminal methyl group bound to an amide moiety to a linker described herein. In some embodiments, 07U is attached via its secondary amine or terminal amino group to a linker described herein. In some embodiments, YCF is attached via either of its terminal hydroxyl groups to a linker described herein. In some embodiments, XK9 is attached via its terminal hydroxyl group to a linker described herein. In some embodiments, NXP is attached via its terminal hydrazone group (NXP) to a linker described herein. In some embodiments, afatinib is attached via its aliphatic amine group to a linker described herein. In some embodiments, fostamatinib is attached via its methoxy group to a linker described herein. In some embodiments, gefitinib is attached via its methoxy group or its ether group to a linker described herein. In some embodiments, lenvatinib is attached via its cyclopropyl group to a linker described herein. In some embodiments, vandetanib is attached via its methoxy group or hydroxyl group to a linker described herein. In some embodiments, vemurafenib is attached via its sulfonyl propyl group to a linker described herein. In some embodiments, gleevec is attached via its amide group or via its aniline amine group to a linker described herein. In some embodiments, pazopanib is attached via its phenyl moiety or via its aniline amine group to a linker described herein. In some embodiments, AT-9283 is attached via its phenyl moiety to a linker described herein. In some embodiments, TAE684 is attached via its phenyl moiety to a linker described herein. In some embodiments, nilotinib is attached via its phenyl moiety or via its aniline amine group to a linker described herein. In some embodiments, crizotinib is attached via its phenyl moiety or diazole group to a linker described herein. In some embodiments, crizotinib is attached via its phenyl moiety or diazole group to a linker described herein. In some embodiments, JNJ FMX is attached via its phenyl moiety to a linker described herein.

In some embodiments, the target protein binding moiety includes a phosphatase inhibitor. In some embodiments, the phosphatase inhibitor is a protein tyrosine phosphatase inhibitor. In some embodiments, the phosphatase inhibitor is an inhibitor of a SHP-2 domain of a tyrosine phosphatase. A non-limiting example of a phosphatase inhibitors includes PTP1B.

In some embodiments, the target protein binding moiety includes an MDM inhibitor. In some embodiments, the MDM inhibitor is an MDM2 inhibitor. Non-limiting examples of MDM2 inhibitors include any one of nutlin-3, nutlin-2, nutlin-1, or trans-4-iodo-4′-boranyl-chalcone. In some embodiments, nutlin-3, nutlin-2, or nutlin-1 is attached via a methoxy group or hydroxyl group to a linker described herein. In some embodiments, trans-4-iodo-4′-boranyl-chalcone is attached via its hydroxyl group to a linker described herein.

In some embodiments, the target protein binding moiety includes a compound that targets a human BET bromodomain-containing protein. In some embodiments, the compound that targets a human BET bromodomain-containing protein is a 3,5-dimethylisoxazole. In some embodiments, the target protein binding moiety includes a compound that inhibits an HDAC. In some embodiments, the target protein binding moiety includes a compound that inhibits a methyltransferase such as a lysine methyltransferase. In some embodiments, the methyltransferase is a human lysine methyltransferase. In some embodiments, the lysine methyltransferase inhibitor is azacytidine. In some embodiments, azacytidine is attached via a hydroxy or amino group to a linker described herein. In some embodiments, the lysine methyltransferase inhibitor is decitabine. In some embodiments, decitabine is attached via a hydroxy or amino group to a linker described herein. In some embodiments, the target protein binding moiety includes an angiogenesis inhibitor. Non-limiting examples of angiogenesis inhibitors include GA-1, estradiol, testosterone, DHT, ovalicin, or fumagillin. In some embodiments, the target protein binding moiety includes an immunosuppressive compound. Non-limiting examples of immunosuppressive compounds include AP21998, a glucocorticoid (e.g., hydrocortisone, prednisone, prednisolone, or methylprednisolone), beclomethasone dipropionate, methotrexate, ciclosporin, tacrolimus, rapamycin, or actinomycin. In some embodiments, the glucocorticoid is attached via a hydroxyl to a linker described herein. In some embodiments, the beclomethasone dipropionate is attached via a propionate to a linker described herein. In some embodiments, methotrexate is attached via either of its terminal hydroxyls to a linker described herein. In some embodiments, ciclosporin is attached via a butyl group to a linker described herein. In some embodiments, tacrolimus is attached via a methoxy group to a linker described herein. In some embodiments, rapamycin is attached via a methoxy group to a linker described herein. In some embodiments, actinomycin is attached via an isopropyl group to a linker described herein. In some embodiments, the target protein binding moiety includes a compound that targets an aryl hydrocarbon receptor (AHR). Non-limiting examples of compounds that target an AHR include apigenin, SRI, or LGC006. In some embodiments, the target protein binding moiety includes a compound that targets a RAF receptor. In some embodiments, the target protein binding moiety includes a compound that targets FKBP. In some embodiments, the target protein binding moiety includes a compound that targets an androgen receptor. Non-limiting examples of compounds that target an androgen receptor include any one of RU59063, SARM, DHT, MDV3100, ARN-509, a hexahydrobenzisoxazole, or a tetramethylcyclobutane. In some embodiments, the target protein binding moiety includes a compound that targets an estrogen receptor. In some embodiments, the target protein binding moiety includes a compound that targets a thyroid hormone receptor. In some embodiments, the target protein binding moiety includes a compound that inhibits an HIV. In some embodiments, the target protein binding moiety includes a compound that inhibits an HIV integrase. In some embodiments, the target protein binding moiety includes a compound that targets an HCV protease. In some embodiments, the target protein binding moiety includes a compound that targets acyl-protein thioesterase-1 and/or -2. Some examples of target protein binding moieties are shown in Table 3. In the table, “R” or a wavy line indicates an optional point of attachment to a linker or other molecule such as a DDB1 binding moiety.

TABLE 3
Target protein binding moieties

| | | Notes (e.g. what |
|---|---|---|
| | | target protein it may |
| Compound | Structure | bind to) |
| A-1 | 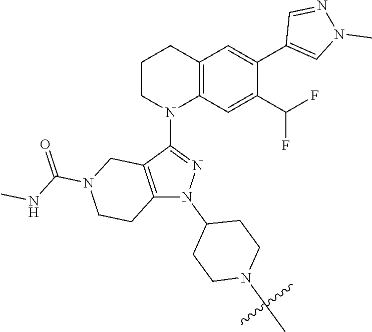 | Binds CBP and/or p300 |
| A-2 | 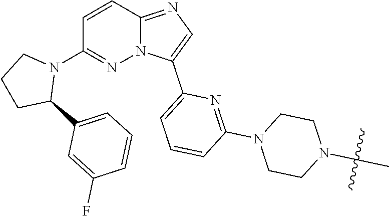 [SMILES: *N1CCN(c2cccc(-c3cnc4ccc(N5CCC[C@@H]5c5cccc(F)c5)nn34)n2)CC1] | Binds TrkA, TrkB, TrkC |
| A-3 | 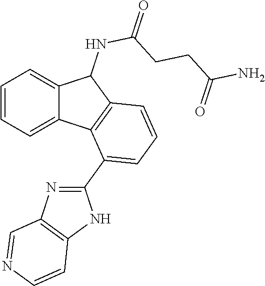 [SMILES: NC(=O)CCC(=O)NC1c2ccccc2-c2c(-c3nc4cnccc4[nH]3)cccc21] | Binds HSP90 |
| A-4 | 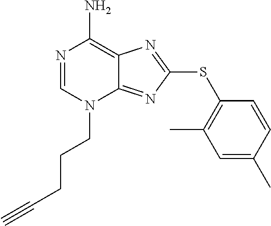 [SMILES: C#CCCCn1cnc(N)c2nc(Sc3ccc(C)cc3C)nc1-2] | Binds HSP90 |
| A-5 | 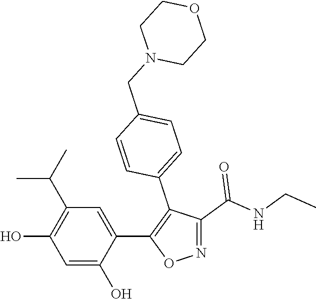 [SMILES: CCNC(=O)c1noc(-c2cc(C(C)C)c(O)cc2O)c1-c1ccc(CN2CCOCC2)cc1] | Binds HSP90 |
| A-6 | 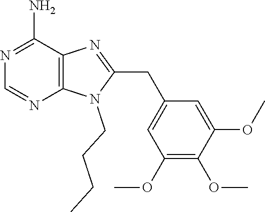 [SMILES: CCCCn1c(Cc2cc(OC)c(OC)c(OC)c2)nc2c(N)ncnc21] | Binds HSP90 |
| A-7 | 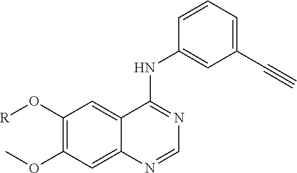 | Binds a tyrosine kinase |
| A-8 | 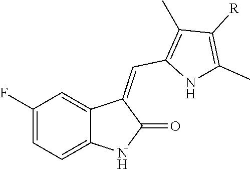 | Binds a kinase |
| A-9 | 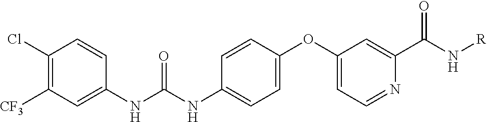 | Binds a kinase |
| A-10 | 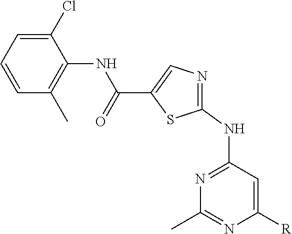 | Binds a kinase |
| A-11 | 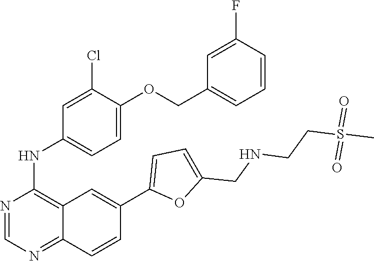 [SMILES: CS(=O)(=O)CCNCc1ccc(-c2ccc3ncnc(Nc4ccc(OCc5cccc(F)c5)c(Cl)c4)c3c2)o1] | Binds a kinase |
| A-12 | 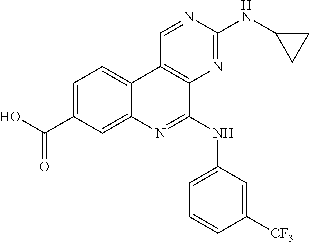 [SMILES: O=C(O)c1ccc2c(c1)nc(Nc1cccc(C(F)(F)F)c1)c1nc(NC3CC3)ncc12] | Binds a kinase |
| A-13 | 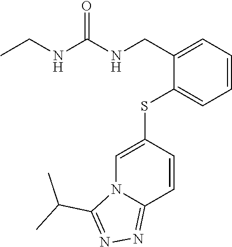 [SMILES: CCNC(=O)NCc1ccccc1Sc1ccc2nnc(C(C)C)n2c1] | Binds a kinase |
| A-14 | 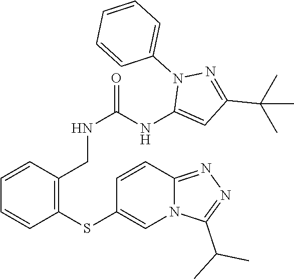 [SMILES: CC(C)c1nnc2ccc(Sc3ccccc3CNC(=O)Nc3cc(C(C)(C)C)nn3-c3ccccc3)cn12] | Binds a kinase |
| A-15 | 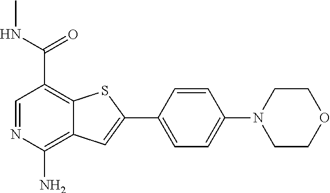 [SMILES: CNC(=O)c1cnc(N)c2cc(-c3ccc(N4CCOCC4)cc3)sc12] | Binds a kinase |
| A-16 | 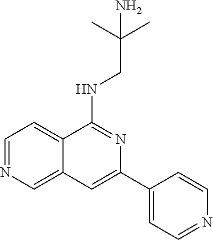 [SMILES: CC(C)(N)CNc1nc(-c2ccncc2)cc2cnccc12] | Binds a kinase |
| A-17 | 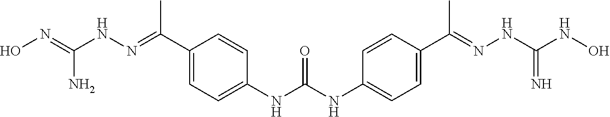 [SMILES: C/C(=N\NC(=N)NO)c1ccc(NC(=O)Nc2ccc(/C(C)=N/N/C(N)=N/O)cc2)cc1] | Binds a kinase |
| A-18 | 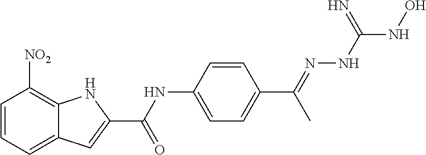 [SMILES: C/C(=N\NC(=N)NO)c1ccc(NC(=O)c2cc3cccc([N+](=O)[O-])c3[nH]2)cc1] | Binds a kinase |
| A-19 | 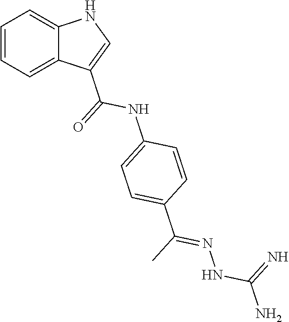 [SMILES: C/C(=N\NC(=N)N)c1ccc(NC(=O)c2c[nH]c3ccccc23)cc1] | Binds a kinase |
| A-20 | 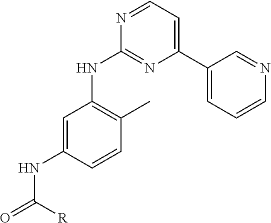 | Binds a kinase |
| A-21 | 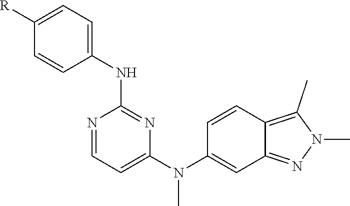 | Binds VEGFR3 |
| A-22 | 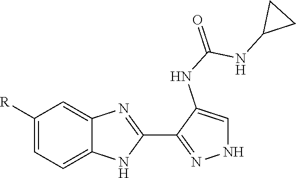 | Binds aurora kinase |
| A-23 | 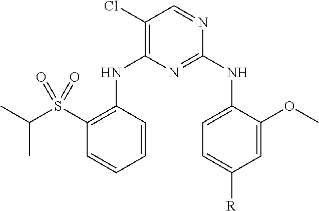 | Binds ALK |
| A-24 | 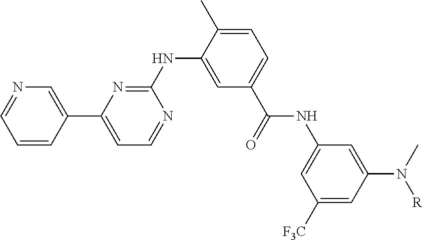 | Binds Abl |
| A-25 | 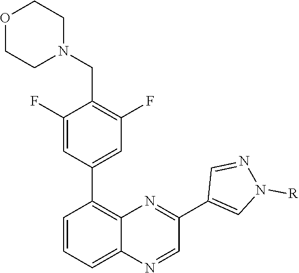 | Binds JAK2 |
| A-26 | 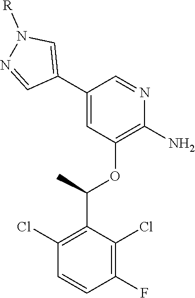 | Binds Alk |
| A-27 | 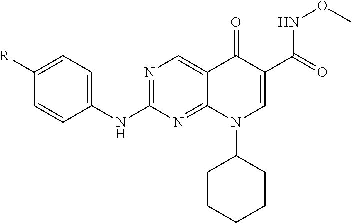 | Binds a kinase |
| A-28 | 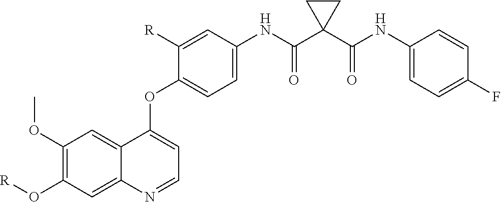 | Binds Met |
| A-29 | 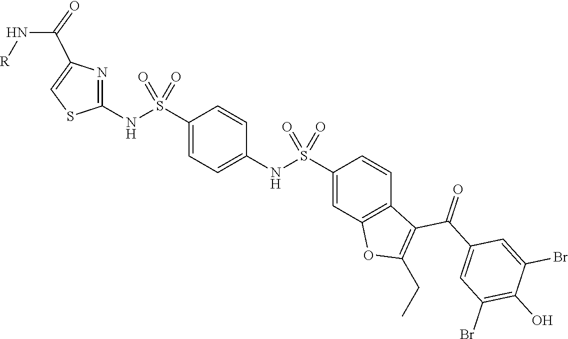 | Binds a tyrosine phosphatase |
| A-30 | 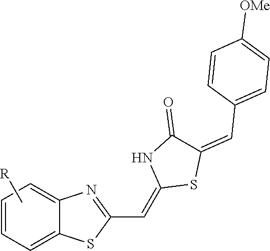 | Binds a tyrosine phosphatase and/or an SHP-2 domain |
| A-31 | 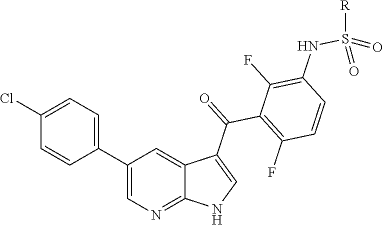 | Binds B-Raf and/or Mek |
| A-32 | 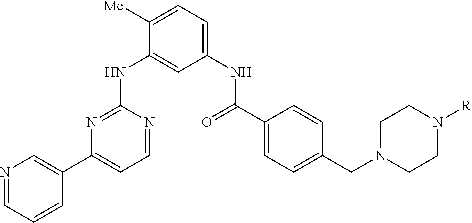 | Binds ABL |
| A-33 | 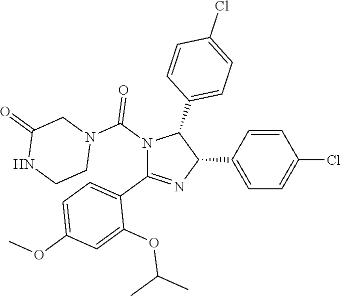 [SMILES: COc1ccc(C2=N[C@@H](c3ccc(Cl)cc3)[C@@H](c3ccc(Cl)cc3)N2C(=O)N2CCNC(=O)C2)c(OC(C)C)c1] | Binds MDM2 |
| A-34 | 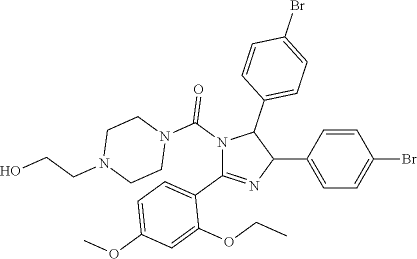 [SMILES: CCOc1cc(OC)ccc1C1=NC(c2ccc(Br)cc2)C(c2ccc(Br)cc2)N1C(=O)N1CCN(CCO)CC1] | Binds MDM2 |
| A-35 | 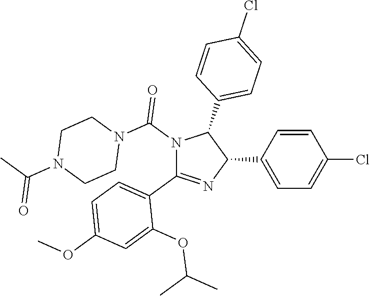 [SMILES: COc1ccc(C2=N[C@@H](c3ccc(Cl)cc3)[C@@H](c3ccc(Cl)cc3)N2C(=O)N2CCN(C(C)=O)CC2)c(OC(C)C)c1] | Binds MDM2 |
| A-36 | 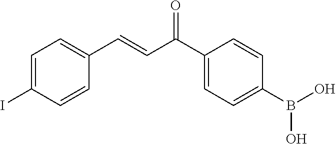 [SMILES: O=C(/C=C/c1ccc(I)cc1)c1ccc(B(O)O)cc1] | Binds MDM2 |
| A-37 | 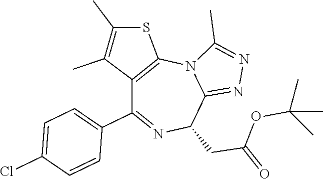 [SMILES: Cc1sc2c(c1C)C(c1ccc(Cl)cc1)=N[C@@H](CC(=O)OC(C)(C)C)c1nnc(C)n1-2] | Binds a human BET bromodomain- containing protein |
| A-38 | 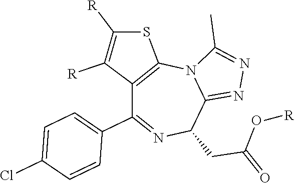 | Binds a human BET bromodomain- containing protein |
| A-39 | 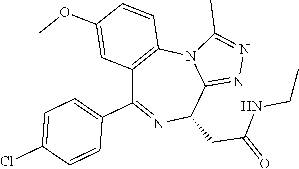 [SMILES: CCNC(=O)C[C@@H]1N=C(c2ccc(Cl)cc2)c2cc(OC)ccc2-n2c(C)nnc21] | Binds a human BET bromodomain- containing protein |
| A-40 | 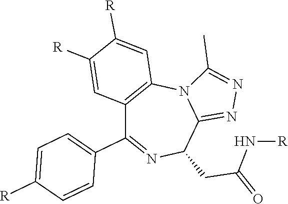 | Binds a human BET bromodomain- containing protein |
| A-41 | 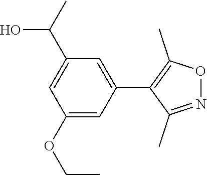 [SMILES: CCOc1cc(-c2c(C)noc2C)cc(C(C)O)c1] | Binds a human BET bromodomain- containing protein |
| A-42 | 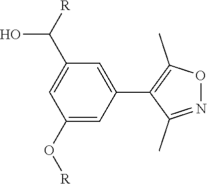 | Binds a human BET bromodomain- containing protein |
| A-43 | 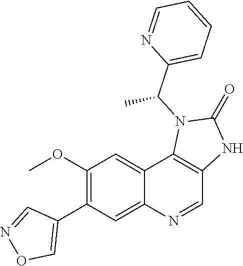 [SMILES: COc1cc2c(cc1-c1cnoc1)ncc1[nH]c(=O)n([C@H](C)c3ccccn3)c12] | Binds a human BET bromodomain- containing protein |
| A-44 | 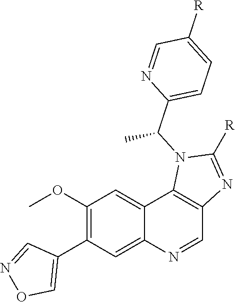 | Binds a human BET bromodomain- containing protein |
| A-45 | 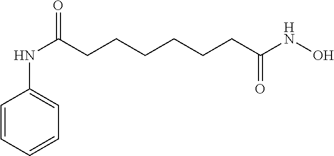 [SMILES: O=C(CCCCCCC(=O)Nc1ccccc1)NO] | Binds an HDAC |
| A-46 | 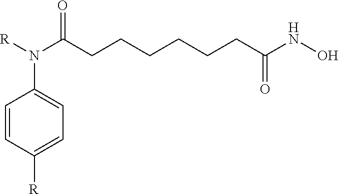 | Binds an HDAC |
| A-47 | 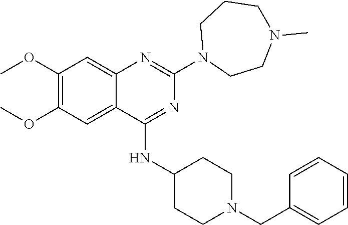 [SMILES: COc1cc2nc(N3CCCN(C)CC3)nc(NC3CCN(Cc4ccccc4)CC3)c2cc1OC] | Binds a lysine methyltransferase |
| A-48 | 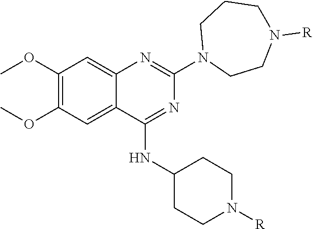 | Binds a lysine methyltransferase |
| A-49 | 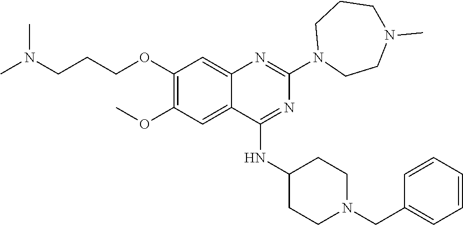 [SMILES: COc1cc2c(NC3CCN(Cc4ccccc4)CC3)nc(N3CCCN(C)CC3)nc2cc1OCCCN(C)C] | Binds a lysine methyltransferase |
| A-50 | 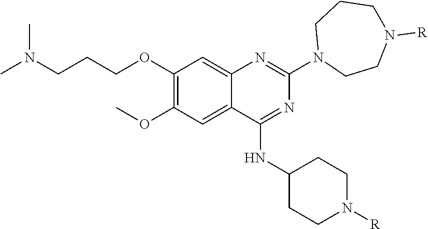 | Binds a lysine methyltransferase |
| A-51 | 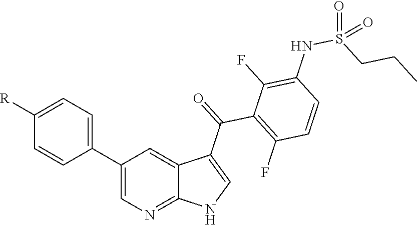 | Binds RAF receptor |
| A-52 | 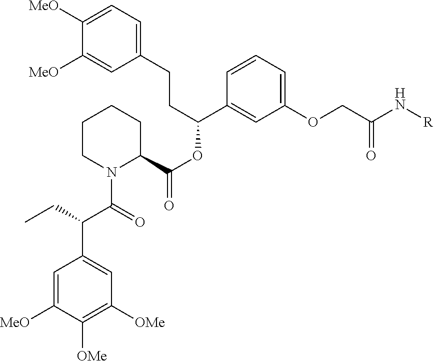 | Binds FKBP |
| A-53 | 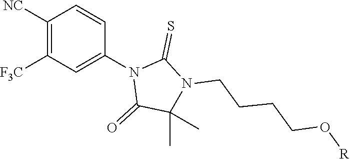 | Binds androgen receptor |
| A-54 | 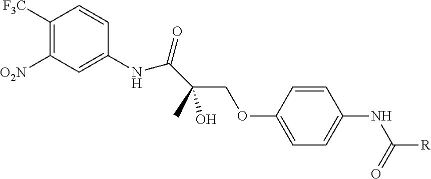 | Binds androgen receptor |
| A-55 | 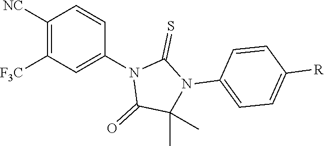 | Binds androgen receptor |
| A-56 | 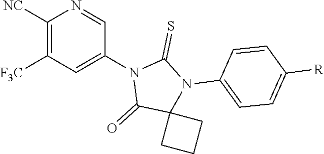 | Binds androgen receptor |
| A-57 | 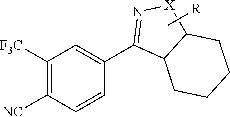 | Binds androgen receptor |
| A-58 | 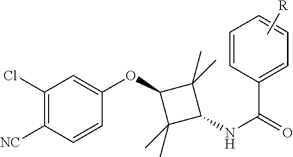 | Binds androgen receptor |
| A-59 | 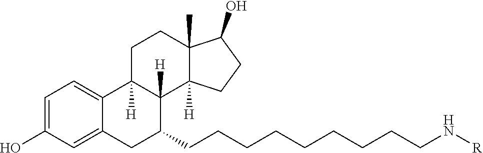 | Binds estrogen receptor |
| A-60 | 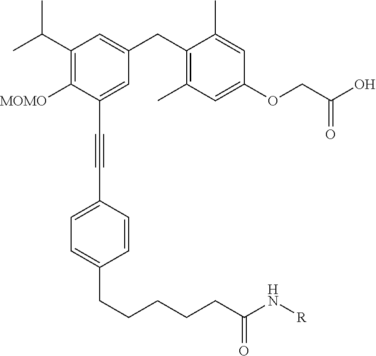 | Binds thyroid hormone receptor; MOMO indicates a methoxymethoxy group |
| A-61 | 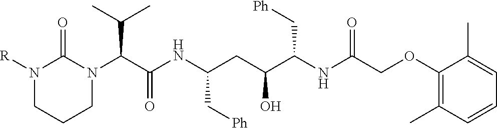 | Binds HIV protease |
| A-62 | 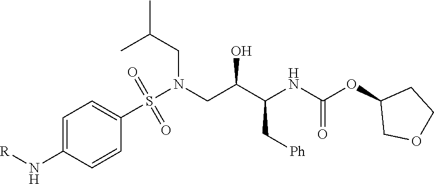 | Binds HIV protease |
| A-63 | 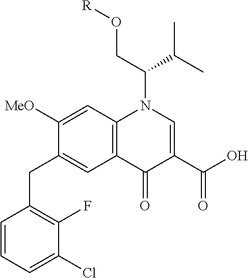 | Binds HIV integrase |
| A-64 | 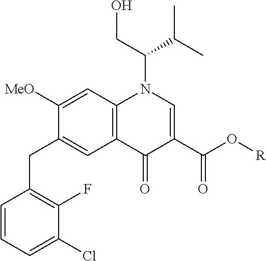 | Binds HIV integrase |
| A-65 | 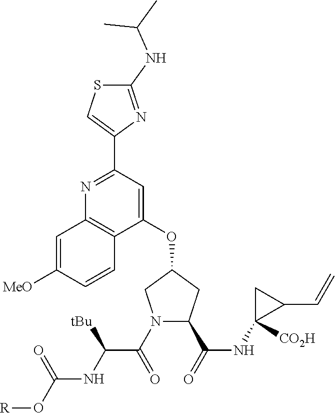 | Binds HCV protease |
| A-66 | 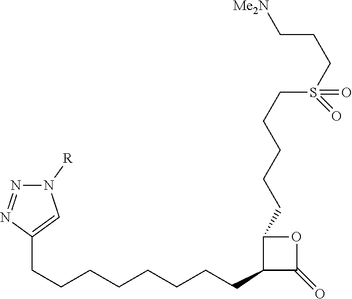 | Binds acyl-protein thioesterase-1 or -2 |
| A-67 | 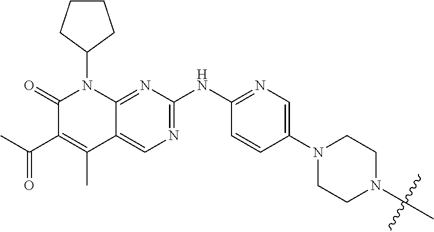 [SMILES: *N1CCN(c2ccc(Nc3ncc4c(C)c(C(C)=O)c(=O)n(C5CCCC5)c4n3)nc2)CC1] | Binds CDK4/6 |
| A-68 | 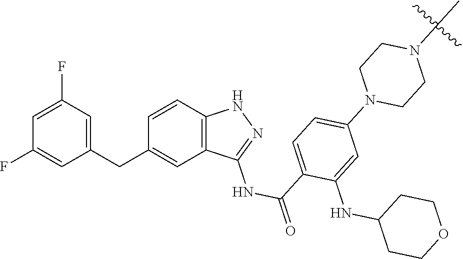 [SMILES: *N1CCN(c2ccc(C(=O)Nc3n[nH]c4ccc(Cc5cc(F)cc(F)c5)cc34)c(NC3CCOCC3)c2)CC1] | Binds TrkA, TrkB, and/or TrkC |
| A-69 | 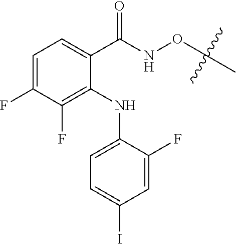 [SMILES: *ONC(=O)c1ccc(F)c(F)c1Nc1ccc(I)cc1F] | Binds Binds MEK1 and/or MEK2 |
| A-70 | 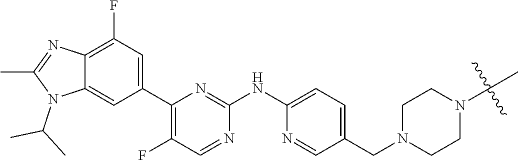 [SMILES: *N1CCN(Cc2ccc(Nc3ncc(F)c(-c4cc(F)c5nc(C)n(C(C)C)c5c4)n3)nc2)CC1] | Binds CDK4/6 |
| A-71 | 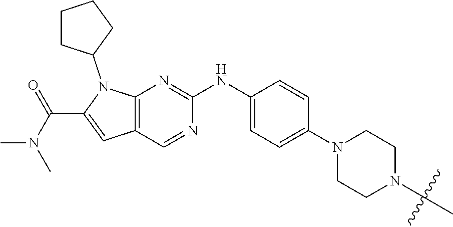 [SMILES: *N1CCN(c2ccc(Nc3ncc4cc(C(=O)N(C)C)n(C5CCCC5)c4n3)cc2)CC1] | Binds CDK4/6 |
| A-72 | 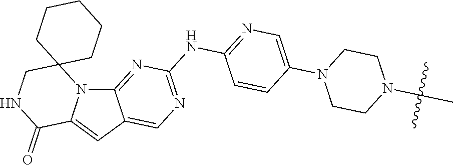 [SMILES: *N1CCN(c2ccc(Nc3ncc4cc5n(c4n3)C3(CCCCC3)CNC5=O)nc2)CC1] | Binds CDK4/6 |
| A-73 | 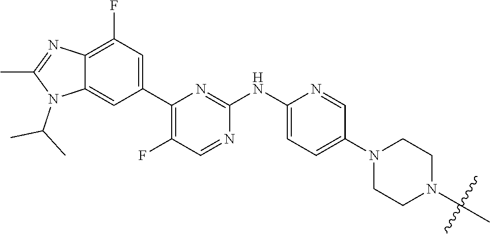 [SMILES: *N1CCN(c2ccc(Nc3ncc(F)c(-c4cc(F)c5nc(C)n(C(C)C)c5c4)n3)nc2)CC1] | Binds CDK4/6 |
| A-74 | 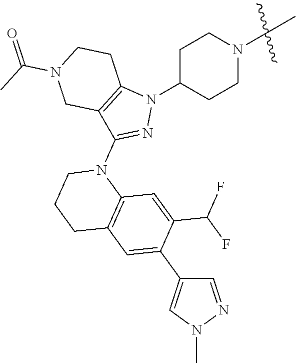 [SMILES: *N1CCC(n2nc(N3CCCc4cc(-c5cnn(C)c5)c(C(F)F)cc43)c3c2CCN(C(C)=O)C3)CC1] | Binds p300 and/or CBP |
| A-75 | 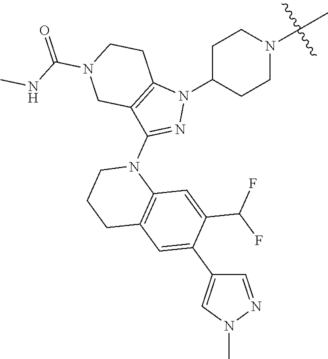 [SMILES: *N1CCC(n2nc(N3CCCc4cc(-c5cnn(C)c5)c(C(F)F)cc43)c3c2CCN(C(=O)NC)C3)CC1] | Binds p300 and/or CBP |
| A-76 | 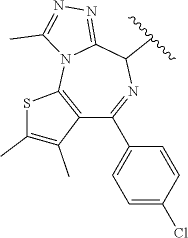 [SMILES: *C1N=C(c2ccc(Cl)cc2)c2c(sc(C)c2C)-n2c(C)nnc21] | Binds BET bromodomain- containing proteins |
| A-77 | 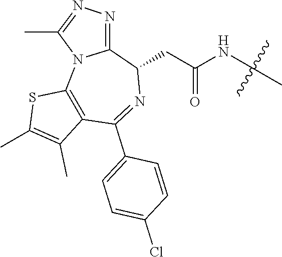 [SMILES: *NC(=O)C[C@@H]1N=C(c2ccc(Cl)cc2)c2c(sc(C)c2C)-n2c(C)nnc21] | Binds BET bromodomain- containing proteins |

### Compounds

In one aspect, provided herein is a heterobifunctional compound of Formula (I), or a pharmaceutically acceptable salt or solvate thereof:

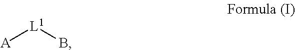

wherein,
A is a target protein binding moiety;
L¹ is a linker; and
B is a DDB1 binding moiety having the structure of Formula (II):

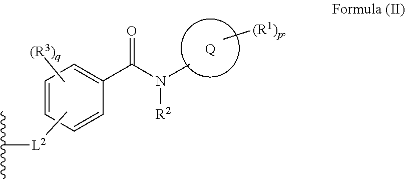

wherein,
   ring Q is phenyl or a 5 or 6-membered monocyclic heteroaryl;
   L² is a bond, —O—, —NR^{4A}—, —NR^{4B}—C(═O)—, —NR^{4B}—C(═O)—(C₁-C₃alkylene)-NR^{4A}—, —NR^{4B}—C(═O)—(C₁-C₃alkylene)-O—, —(C₁-C₃alkylene)-NR^{4B}—C(═O)—, —C(═O)NR^{4A}—, —C₁-C₃alkylene-, —C₂-C₃ alkenylene-, —C₂-C₃alkynylene-, C₃-C₈ cycloalkylene, or C₂-C₈ heterocyclene;
R¹ is hydrogen, halogen, —CN, NO₂, —OR^{4A}, —NR^{4A}R^{4B}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl or heteroaryl, or
two R¹, together with the atom(s) to which they are connected, optionally form C₃-C₁₃ cycloalkyl, C₂-C₁₂ heterocyclyl, aryl, or heteroaryl;
R² is hydrogen, C₁-C₆ alkyl, C₃-C₈ cycloalkyl, OH, or O—C₁-C₄ alkyl;
each R³ is independently hydrogen, halogen, —CN, —NO₂, —OR^{4A}, —NR^{4A}R^{4B}, —C(═O)R^{4A}, —C(═O)OR^{4A}, —C(═O)NR^{4B}R^{4A}, —OC(═O)R^{4A}, —N(R^{4A})C(═O)R^{4B}, C_-C₆ alkyl, C₁-C₆ haloalkyl, C₁-C₆ heteroalkyl, C₃-C₈ cycloalkyl, C₂-C₈ heterocyclyl, aryl, or heteroaryl, or
two R³, together with the atom(s) to which they are connected, optionally form C₃-C₁₃ cycloalkyl, C₂-C₁₂ heterocyclyl, aryl, or heteroaryl;
each R^{4A} and R^{4B} is independently hydrogen, C₁-C₆ alkyl, C₂-C₆ alkenyl, C₂-C₆ alkynyl, C₁-C₆ haloalkyl, C₁-C₆ heteroalkyl, C₃-C₈ cycloalkyl, C₂-C₈ heterocyclyl, aryl, or heteroaryl, or R^{4A} and R^{4B}, together with the atom(s) to which they are connected, optionally form C₂-C₁₂ heterocyclyl;
p is 1, 2 or 3; and
q is 1, 2 or 3.

In some embodiments, the compound comprises a heterobifunctional compound. In some embodiments, the heterobifunctional compound is a compound described in Table 4, or a pharmaceutically acceptable salt or solvate thereof.

TABLE 4
Representative heterobifunctional compounds.

| Cpd. No. | Structure and Chemical Name |
|---|---|
| CPD-001 | 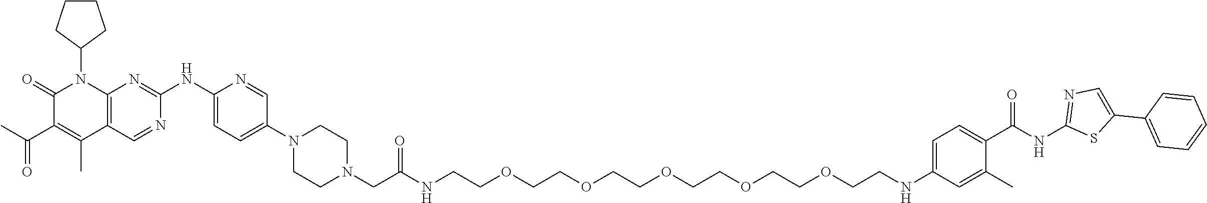 [SMILES: CC(=O)c1c(C)c2cnc(Nc3ccc(N4CCN(CC(=O)NCCOCCOCCOCCOCCOCCNc5ccc(C(=O)Nc6ncc(-c7ccccc7)s6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-2-methyl-N-(5-phenylthiazol-2-yl)benzamide |
| CPD-001 | 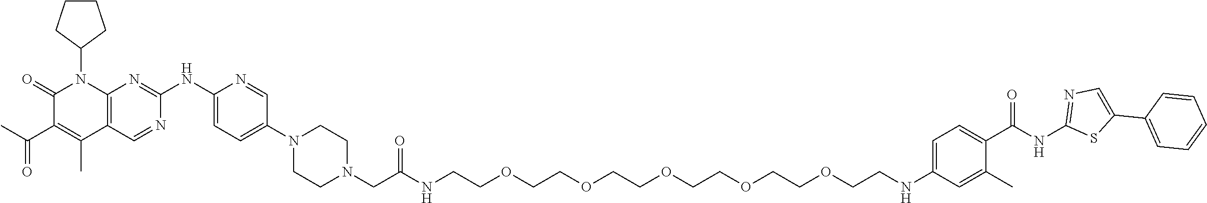 [SMILES: CC(=O)c1c(C)c2cnc(Nc3ccc(N4CCN(CC(=O)NCCOCCOCCOCCOCCOCCNc5ccc(C(=O)Nc6ncc(-c7ccccc7)s6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-2-methyl-N-(5-phenylthiazol-2-yl)benzamide |
| CPD-002 | 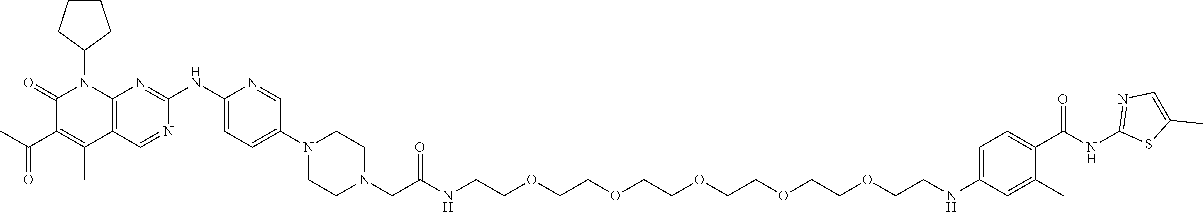 [SMILES: CC(=O)c1c(C)c2cnc(Nc3ccc(N4CCN(CC(=O)NCCOCCOCCOCCOCCOCCNc5ccc(C(=O)Nc6ncc(C)s6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-2-methyl-N-(5-methylthiazol-2-yl)benzamide |
| CPD-003 | 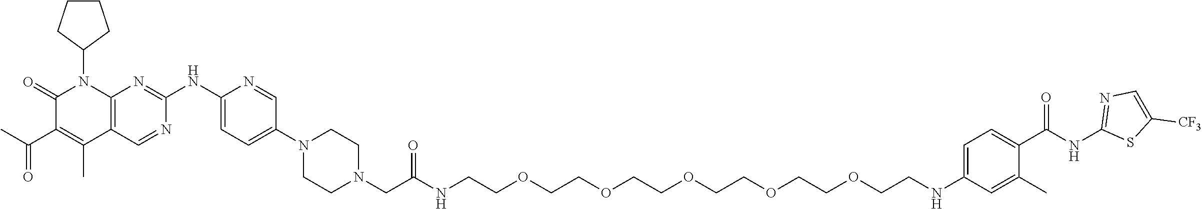 [SMILES: CC(=O)c1c(C)c2cnc(Nc3ccc(N4CCN(CC(=O)NCCOCCOCCOCCOCCOCCNc5ccc(C(=O)Nc6ncc(C(F)(F)F)s6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-2-methyl-N-(5-(trifluoromethyl)thiazol-2-yl)benzamide |
| CPD-004 | 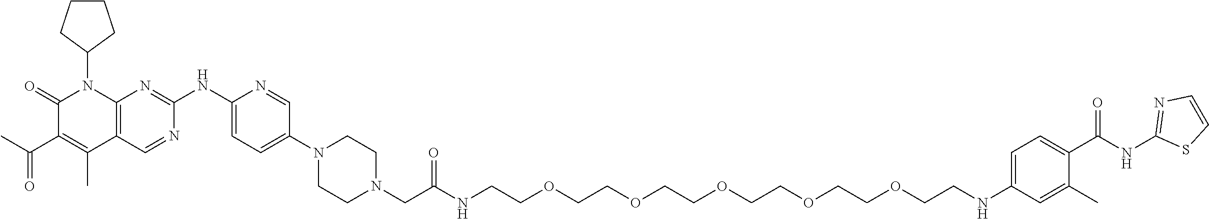 [SMILES: CC(=O)c1c(C)c2cnc(Nc3ccc(N4CCN(CC(=O)NCCOCCOCCOCCOCCOCCNc5ccc(C(=O)Nc6nccs6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-2-methyl-N-(thiazol-2-yl)benzamide |
| CPD-005 | 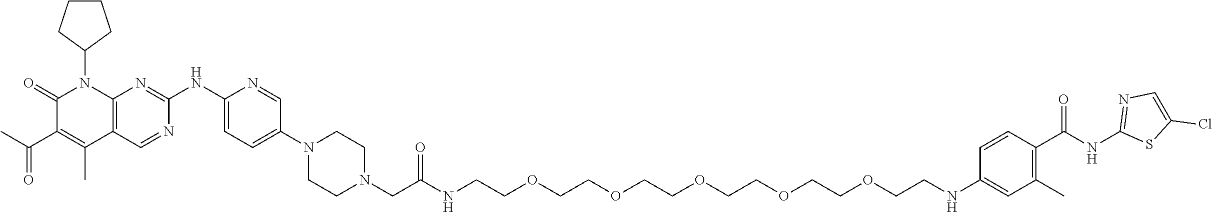 [SMILES: CC(=O)c1c(C)c2cnc(Nc3ccc(N4CCN(CC(=O)NCCOCCOCCOCCOCCOCCNc5ccc(C(=O)Nc6ncc(Cl)s6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-N-(5-chlorothiazol-2-yl)-2-methylbenzamide |
| CPD-006 | 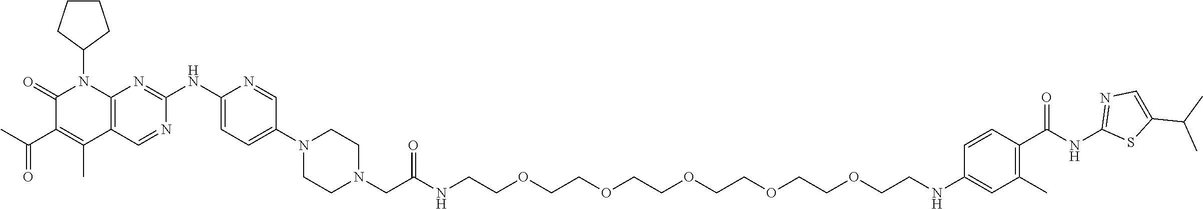 [SMILES: CC(=O)c1c(C)c2cnc(Nc3ccc(N4CCN(CC(=O)NCCOCCOCCOCCOCCOCCNc5ccc(C(=O)Nc6ncc(C(C)C)s6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-N-(5-isopropylthiazol-2-yl)-2-methylbenzamide |
| CPD-007 | 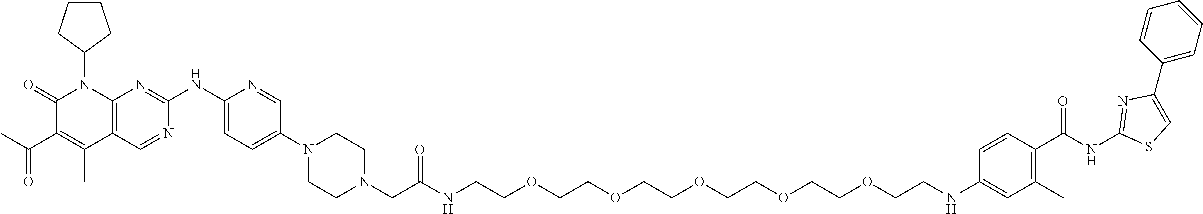 [SMILES: CC(=O)c1c(C)c2cnc(Nc3ccc(N4CCN(CC(=O)NCCOCCOCCOCCOCCOCCNc5ccc(C(=O)Nc6nc(-c7ccccc7)cs6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-2-methyl-N-(4-phenylthiazol-2-yl)benzamide |
| CPD-008 | 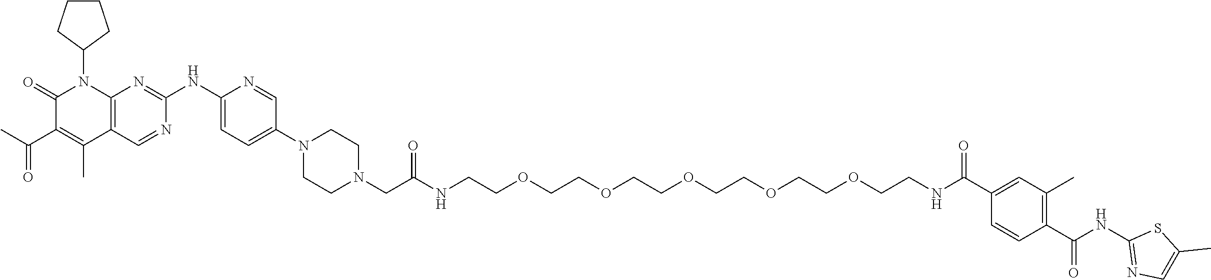 [SMILES: CC(=O)c1c(C)c2cnc(Nc3ccc(N4CCN(CC(=O)NCCOCCOCCOCCOCCOCCNC(=O)c5ccc(C(=O)Nc6ncc(C)s6)c(C)c5)CC4)cn3)nc2n(C2CCCC2)c1=O] N⁴-(1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)-2-methyl-N¹-(5-methylthiazol-2-yl)terephthalamide |
| CPD-009 | 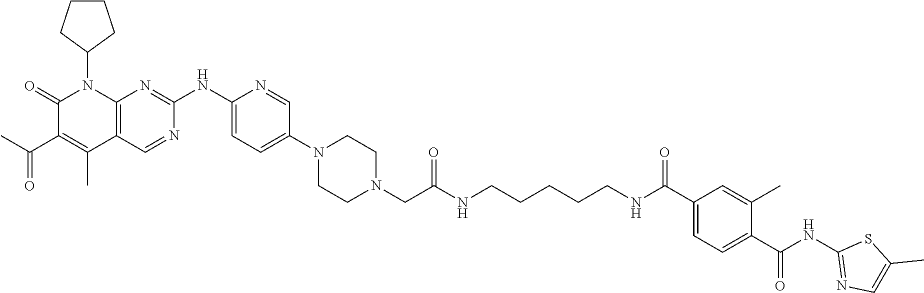 [SMILES: CC(=O)c1c(C)c2cnc(Nc3ccc(N4CCN(CC(=O)NCCCCCNC(=O)c5ccc(C(=O)Nc6ncc(C)s6)c(C)c5)CC4)cn3)nc2n(C2CCCC2)c1=O] N⁴-(5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentyl)-2-methyl-N¹- (5-methylthiazol-2-yl)terephthalamide |
| CPD-010 | 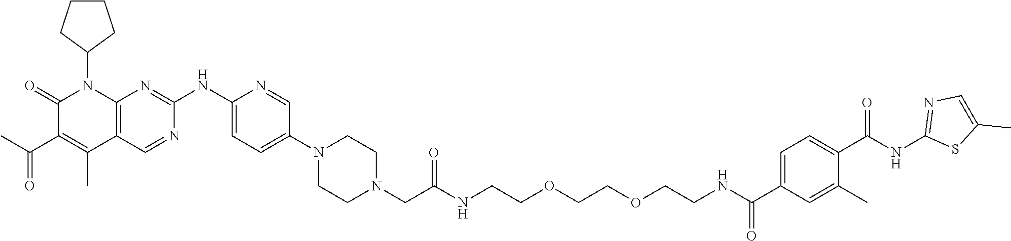 [SMILES: CC(=O)c1c(C)c2cnc(Nc3ccc(N4CCN(CC(=O)NCCOCCOCCNC(=O)c5ccc(C(=O)Nc6ncc(C)s6)c(C)c5)CC4)cn3)nc2n(C2CCCC2)c1=O] N⁴-(2-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)ethoxy)ethoxy)ethyl)- 2-methyl-N¹-(5-methylthiazol-2-yl)terephthalamide |
| CPD-011 | 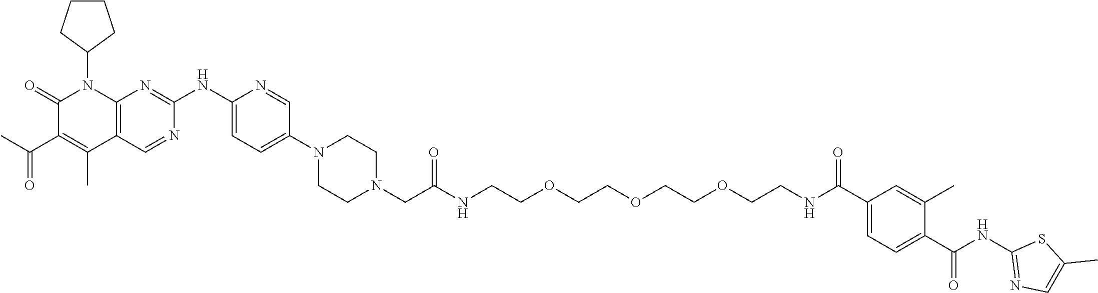 [SMILES: CC(=O)c1c(C)c2cnc(Nc3ccc(N4CCN(CC(=O)NCCOCCOCCOCCNC(=O)c5ccc(C(=O)Nc6ncc(C)s6)c(C)c5)CC4)cn3)nc2n(C2CCCC2)c1=O] N⁴-(1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12-trioxa-3- azatetradecan-14-yl)-2-methyl-N¹-(5-methylthiazol-2-yl)terephthalamide |
| CPD-012 | 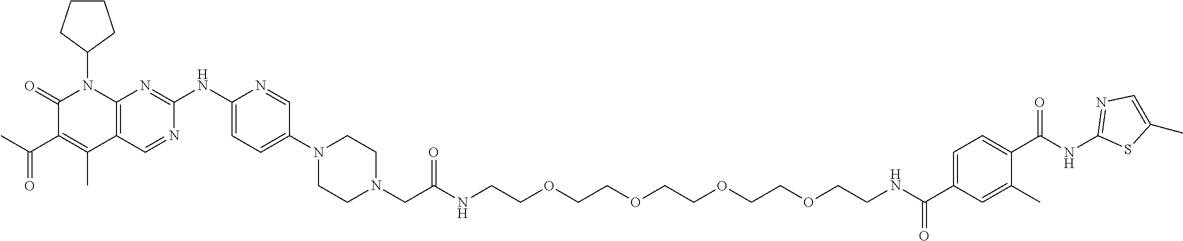 [SMILES: CC(=O)c1c(C)c2cnc(Nc3ccc(N4CCN(CC(=O)NCCOCCOCCOCCOCCNC(=O)c5ccc(C(=O)Nc6ncc(C)s6)c(C)c5)CC4)cn3)nc2n(C2CCCC2)c1=O] N⁴-(1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15-tetraoxa-3- azaheptadecan-17-yl)-2-methyl-N¹⁻(5-methylthiazol-2-yl)terephthalamide |
| CPD-013 | 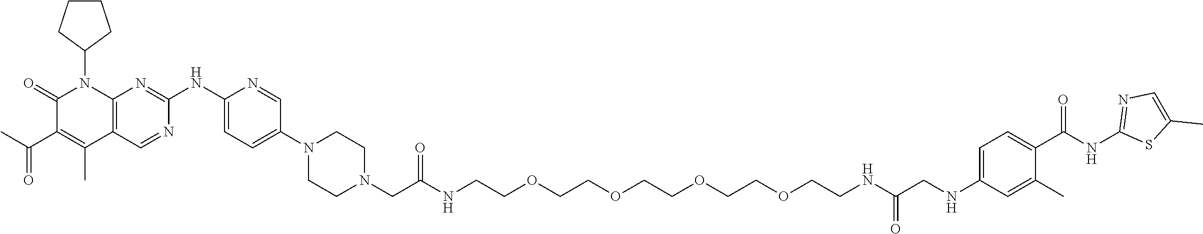 [SMILES: CC(=O)c1c(C)c2cnc(Nc3ccc(N4CCN(CC(=O)NCCOCCOCCOCCOCCNC(=O)CNc5ccc(C(=O)Nc6ncc(C)s6)c(C)c5)CC4)cn3)nc2n(C2CCCC2)c1=O] 4-((20-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2,19-dioxo-6,9,12,15-tetraoxa- 3,18-diazaicosyl)amino)-2-methyl-N-(5-methylthiazol-2-yl)benzamide |
| CPD-014 | 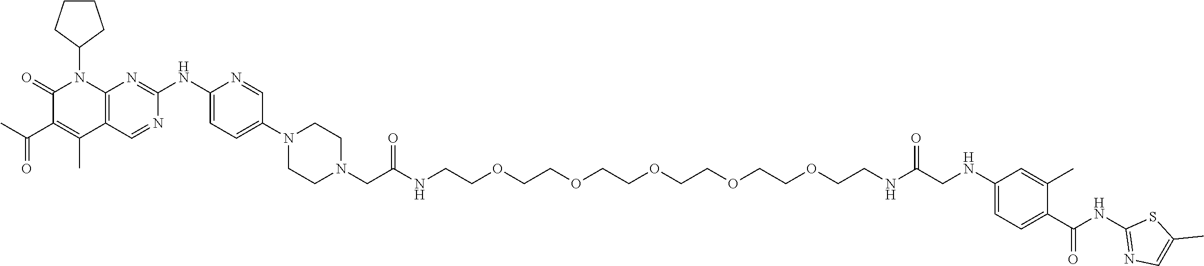 [SMILES: CC(=O)c1c(C)c2cnc(Nc3ccc(N4CCN(CC(=O)NCCOCCOCCOCCOCCOCCNC(=O)CNc5ccc(C(=O)Nc6ncc(C)s6)c(C)c5)CC4)cn3)nc2n(C2CCCC2)c1=O] 4-((23-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2,22-dioxo-6,9,12,15,18- pentaoxa-3,21-diazatricosyl)amino)-2-methyl-N-(5-methylthiazol-2-yl)benzamide |
| CPD-015 | 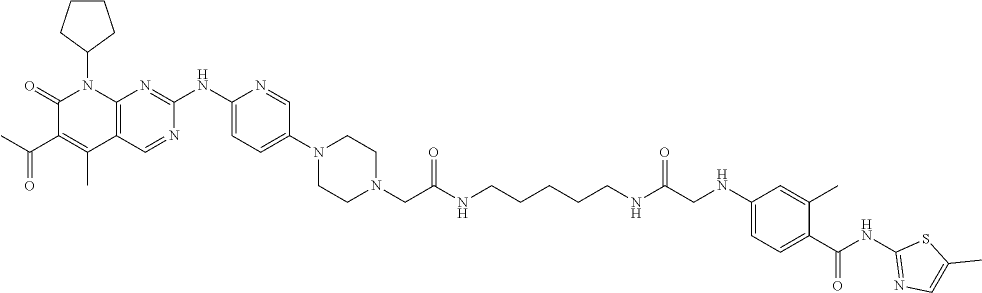 [SMILES: CC(=O)c1c(C)c2cnc(Nc3ccc(N4CCN(CC(=O)NCCCCCNC(=O)CNc5ccc(C(=O)Nc6ncc(C)s6)c(C)c5)CC4)cn3)nc2n(C2CCCC2)c1=O] 4-((2-((5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentyl)amino)-2- oxoethyl)amino)-2-methyl-N-(5-methylthiazol-2-yl)benzamide |
| CPD-016 | 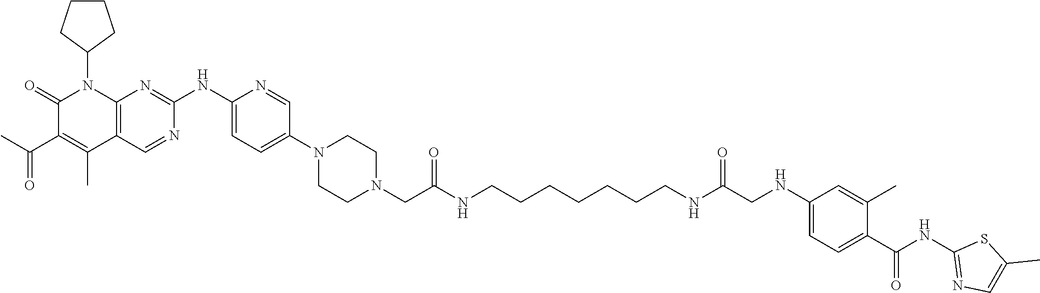 [SMILES: CC(=O)c1c(C)c2cnc(Nc3ccc(N4CCN(CC(=O)NCCCCCCCNC(=O)CNc5ccc(C(=O)Nc6ncc(C)s6)c(C)c5)CC4)cn3)nc2n(C2CCCC2)c1=O] 4-((2-((7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptyl)amino)-2- oxoethyl)amino)-2-methyl-N-(5-methylthiazol-2-yl)benzamide |
| CPD-017 | 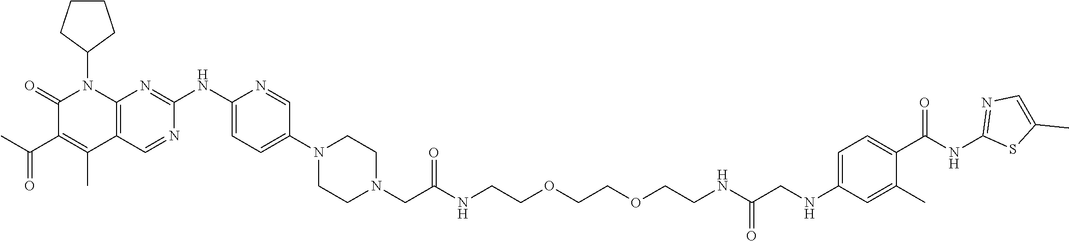 [SMILES: CC(=O)c1c(C)c2cnc(Nc3ccc(N4CCN(CC(=O)NCCOCCOCCNC(=O)CNc5ccc(C(=O)Nc6ncc(C)s6)c(C)c5)CC4)cn3)nc2n(C2CCCC2)c1=O] 4-((14-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2,13-dioxo-6,9-dioxa-3,12- diazatetradecyl)amino)-2-methyl-N-(5-methylthiazol-2-yl)benzamide |
| CPD-018 | 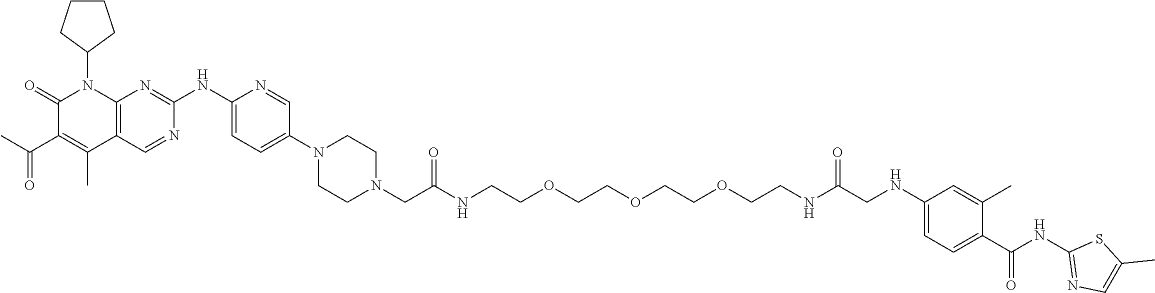 [SMILES: CC(=O)c1c(C)c2cnc(Nc3ccc(N4CCN(CC(=O)NCCOCCOCCOCCNC(=O)CNc5ccc(C(=O)Nc6ncc(C)s6)c(C)c5)CC4)cn3)nc2n(C2CCCC2)c1=O] 4-((17-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2,16-dioxo-6,9,12-trioxa-3,15- diazaheptadecyl)amino)-2-methyl-N-(5-methylthiazol-2-yl)benzamide |
| CPD-019 | 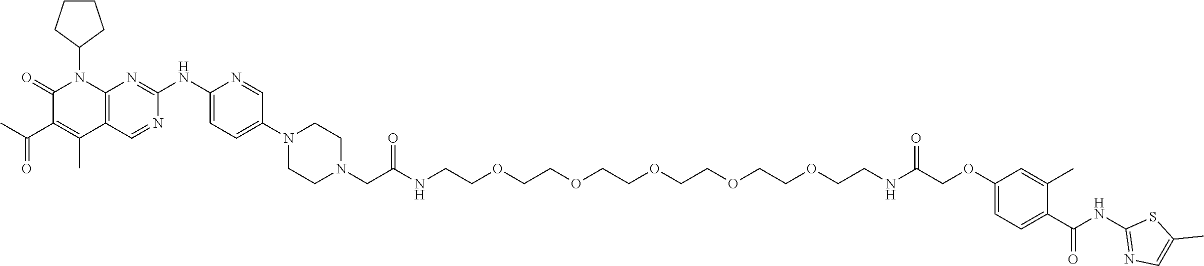 [SMILES: CC(=O)c1c(C)c2cnc(Nc3ccc(N4CCN(CC(=O)NCCOCCOCCOCCOCCOCCNC(=O)COc5ccc(C(=O)Nc6ncc(C)s6)c(C)c5)CC4)cn3)nc2n(C2CCCC2)c1=O] 4-((23-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2,22-dioxo-6,9,12,15,18- pentaoxa-3,21-diazatricosyl)oxy)-2-methyl-N-(5-methylthiazol-2-yl)benzamide |
| CPD-020 | 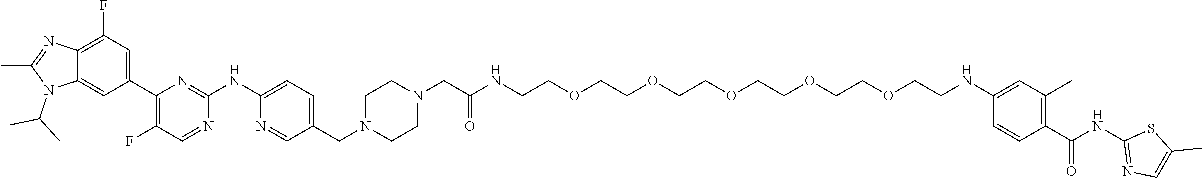 4-((1-(4-((6-((5-fluoro-4-(4-fluoro-1-isopropyl-2-methyl-1H-benzo[d]imidazol-6- yl)pyrimidin-2-yl)amino)pyridin-3-yl)methyl)piperazin-1-yl)-2-oxo-6,9,12,15,18- pentaoxa-3-azaicosan-20-yl)amino)-2-methyl-N-(5-methylthiazol-2-yl)benzamide |
| CPD-021 | 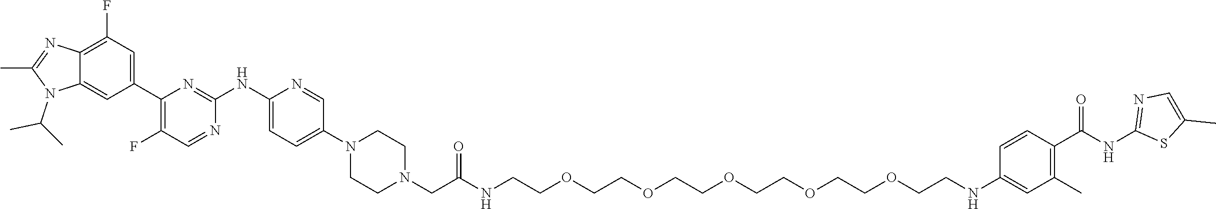 4-((1-(4-(6-((5-fluoro-4-(4-fluoro-1-isopropyl-2-methyl-1H-benzo[d]imidazol-6- yl)pyrimidin-2-yl)amino)pyridin-3-yl)piperazin-1-yl)-2-oxo-6,9,12,15,18-pentaoxa-3- azaicosan-20-yl)amino)-2-methyl-N-(5-methylthiazol-2-yl)benzamide |
| CPD-022 | 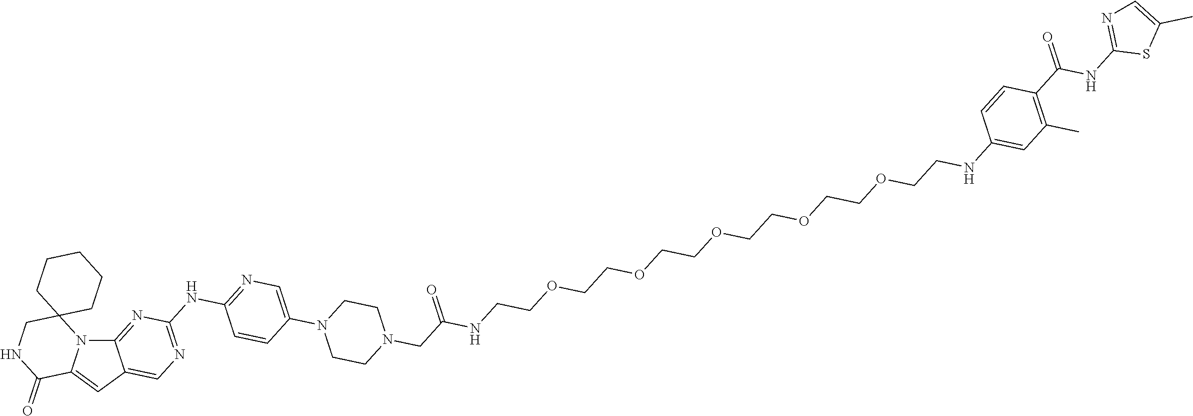 [SMILES: Cc1cnc(NC(=O)c2ccc(NCCOCCOCCOCCOCCOCCNC(=O)CN3CCN(c4ccc(Nc5ncc6cc7n(c6n5)C5(CCCCC5)CNC7=O)nc4)CC3)cc2C)s1] 2-methyl-N-(5-methylthiazol-2-yl)-4-((2-oxo-1-(4-(6-((6′-oxo-7′,8′-dihydro-6′H- spiro[cyclohexane-1,9′-pyrazino[1′,2′:1,5]pyrrolo[2,3-d]pyrimidin]-2′- yl)amino)pyridin-3-yl)piperazin-1-yl)-6,9,12,15,18-pentaoxa-3-azaicosan-20- yl)amino)benzamide |
| CPD-023 | 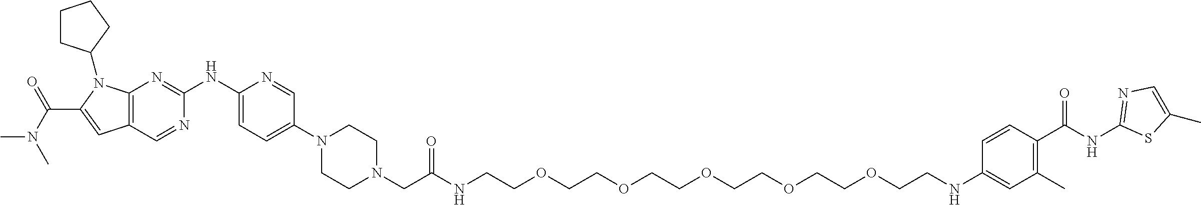 7-cyclopentyl-N,N-dimethyl-2-((5-(4-(20-((3-methyl-4-((5-methylthiazol-2- yl)carbamoyl)phenyl)amino)-2-oxo-6,9,12,15,18-pentaoxa-3-azaicosyl)piperazin-1- yl)pyridin-2-yl)amino)-7H-pyrrolo[2,3-d]pyrimidine-6-carboxamide |
| CPD-024 | 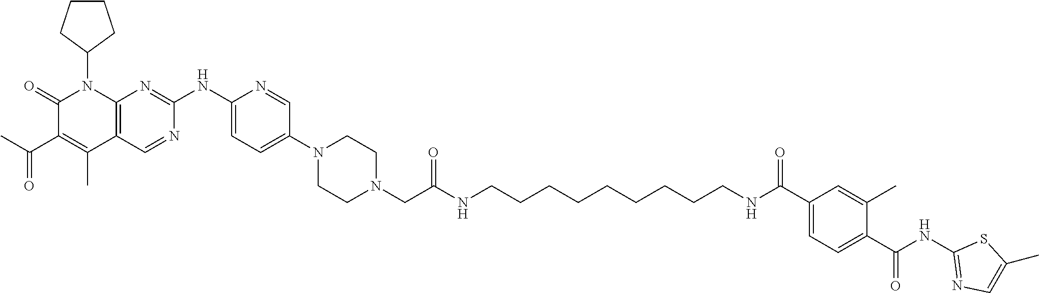 [SMILES: CC(=O)c1c(C)c2cnc(Nc3ccc(N4CCN(CC(=O)NCCCCCCCCCNC(=O)c5ccc(C(=O)Nc6ncc(C)s6)c(C)c5)CC4)cn3)nc2n(C2CCCC2)c1=O] N⁴-(9-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)nonyl)-2-methyl-N¹- (5-methylthiazol-2-yl)terephthalamide |
| CPD-025 | 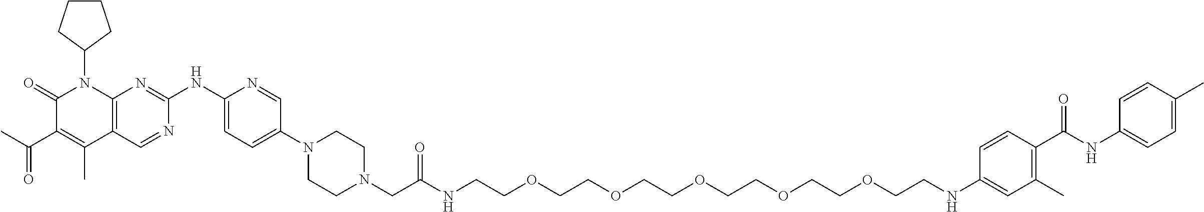 [SMILES: CC(=O)c1c(C)c2cnc(Nc3ccc(N4CCN(CC(=O)NCCOCCOCCOCCOCCOCCNc5ccc(C(=O)Nc6ccc(C)cc6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-2-methyl-N-(p-tolyl)benzamide |
| CPD-026 | 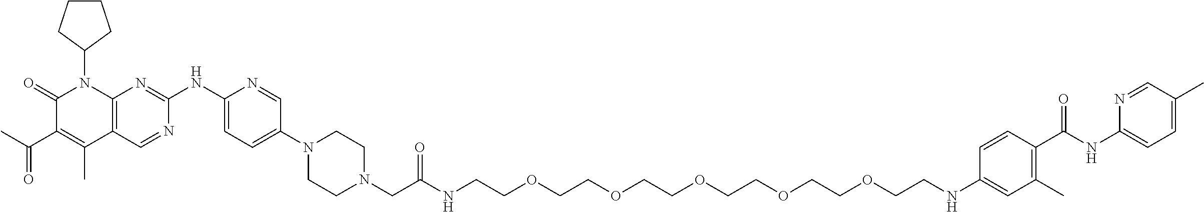 [SMILES: CC(=O)c1c(C)c2cnc(Nc3ccc(N4CCN(CC(=O)NCCOCCOCCOCCOCCOCCNc5ccc(C(=O)Nc6ccc(C)cn6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-2-methyl-N-(5-methylpyridin-2-yl)benzamide |
| CPD-027 | 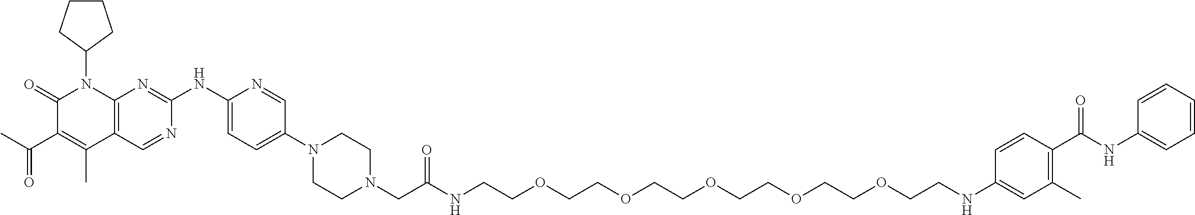 [SMILES: CC(=O)c1c(C)c2cnc(Nc3ccc(N4CCN(CC(=O)NCCOCCOCCOCCOCCOCCNc5ccc(C(=O)Nc6ccccc6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-2-methyl-N-phenylbenzamide |
| CPD-028 | 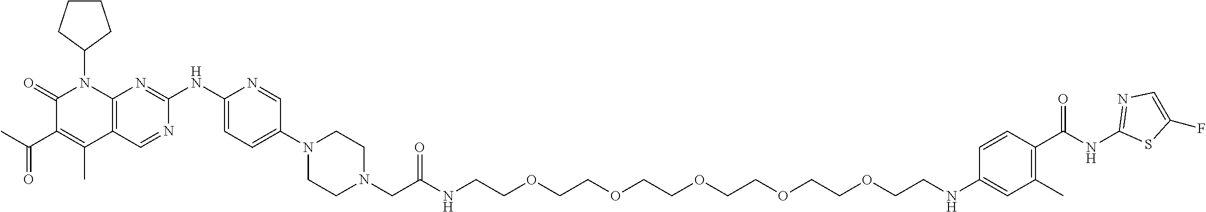 [SMILES: CC(=O)c1c(C)c2cnc(Nc3ccc(N4CCN(CC(=O)NCCOCCOCCOCCOCCOCCNc5ccc(C(=O)Nc6ncc(F)s6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-N-(5-fluorothiazol-2-yl)-2-methylbenzamide |
| CPD-029 | 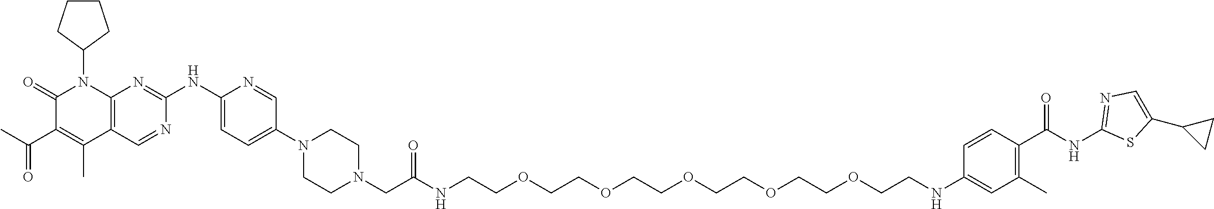 [SMILES: CC(=O)c1c(C)c2cnc(Nc3ccc(N4CCN(CC(=O)NCCOCCOCCOCCOCCOCCNc5ccc(C(=O)Nc6ncc(C7CC7)s6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-N-(5-cyclopropylthiazol-2-yl)-2-methylbenzamide |
| CPD-030 | 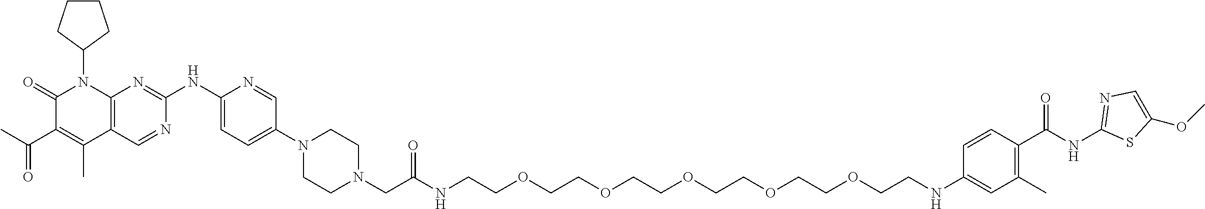 [SMILES: COc1cnc(NC(=O)c2ccc(NCCOCCOCCOCCOCCOCCNC(=O)CN3CCN(c4ccc(Nc5ncc6c(C)c(C(C)=O)c(=O)n(C7CCCC7)c6n5)nc4)CC3)cc2C)s1] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-N-(5-methoxythiazol-2-yl)-2-methylbenzamide |
| CPD-031 | 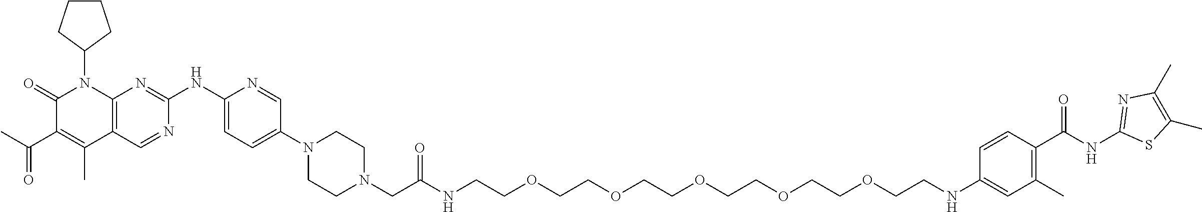 [SMILES: CC(=O)c1c(C)c2cnc(Nc3ccc(N4CCN(CC(=O)NCCOCCOCCOCCOCCOCCNc5ccc(C(=O)Nc6nc(C)c(C)s6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-N-(4,5-dimethylthiazol-2-yl)-2-methylbenzamide |
| CPD-032 | 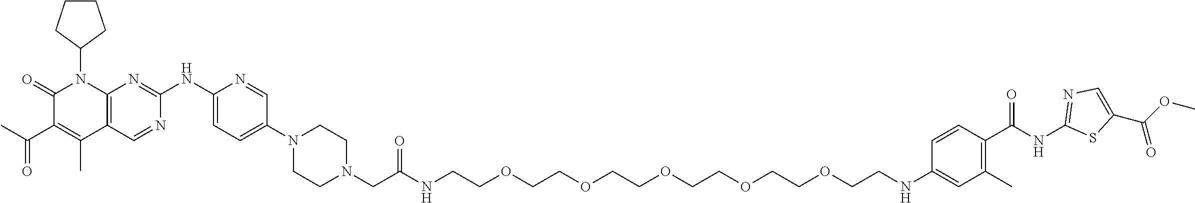 [SMILES: COC(=O)c1cnc(NC(=O)c2ccc(NCCOCCOCCOCCOCCOCCNC(=O)CN3CCN(c4ccc(Nc5ncc6c(C)c(C(C)=O)c(=O)n(C7CCCC7)c6n5)nc4)CC3)cc2C)s1] methyl 2-(4-((1-(4-(6-((6-acetyl-8-cyclopentyl-5-methyl-7-oxo-7,8- dihydropyrido[2,3-d]pyrimidin-2-yl)amino)pyridin-3-yl)piperazin-1-yl)-2-oxo- 6,9,12,15,18-pentaoxa-3-azaicosan-20-yl)amino)-2-methylbenzamido)thiazole-5- carboxylate |
| CPD-033 | 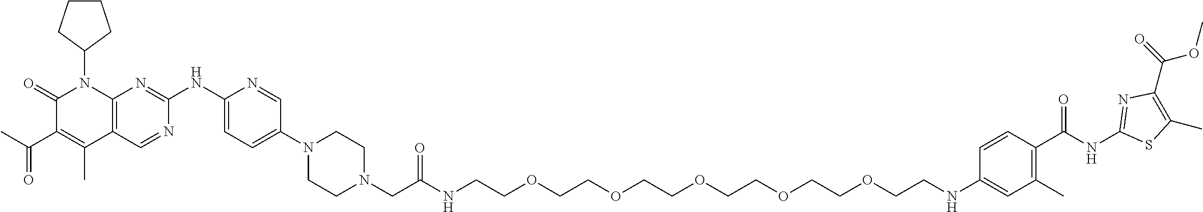 [SMILES: COC(=O)c1nc(NC(=O)c2ccc(NCCOCCOCCOCCOCCOCCNC(=O)CN3CCN(c4ccc(Nc5ncc6c(C)c(C(C)=O)c(=O)n(C7CCCC7)c6n5)nc4)CC3)cc2C)sc1C] methyl 2-(4-((1-(4-(6-((6-acetyl-8-cyclopentyl-5-methyl-7-oxo-7,8- dihydropyrido[2,3-d]pyrimidin-2-yl)amino)pyridin-3-yl)piperazin-1-yl)-2-oxo- 6,9,12,15,18-pentaoxa-3-azaicosan-20-yl)amino)-2-methylbenzamido)-5- methylthiazole-4-carboxylate |
| CPD-034 | 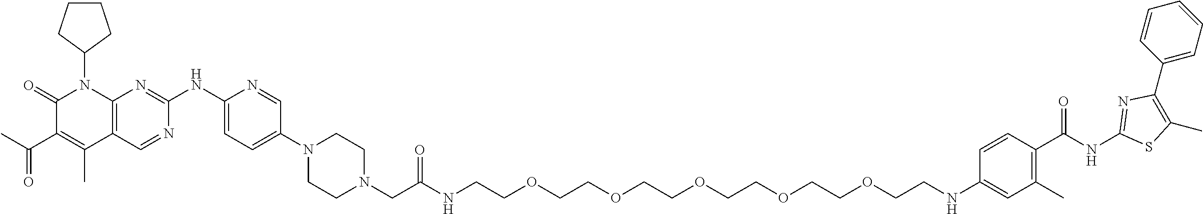 [SMILES: CC(=O)c1c(C)c2cnc(Nc3ccc(N4CCN(CC(=O)NCCOCCOCCOCCOCCOCCNc5ccc(C(=O)Nc6nc(-c7ccccc7)c(C)s6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-2-methyl-N-(5-methyl-4-phenylthiazol-2-yl)benzamide |
| CPD-035 | 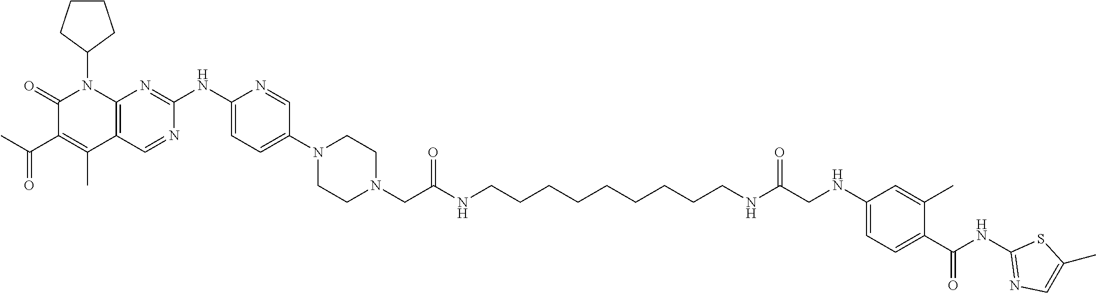 [SMILES: CC(=O)c1c(C)c2cnc(Nc3ccc(N4CCN(CC(=O)NCCCCCCCCCNC(=O)CNc5ccc(C(=O)Nc6ncc(C)s6)c(C)c5)CC4)cn3)nc2n(C2CCCC2)c1=O] 4-((2-((9-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)nonyl)amino)-2- oxoethyl)amino)-2-methyl-N-(5-methylthiazol-2-yl)benzamide |
| CPD-036 | 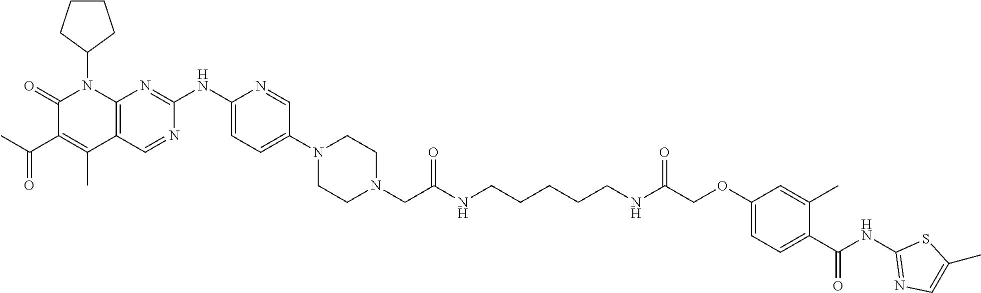 [SMILES: CC(=O)c1c(C)c2cnc(Nc3ccc(N4CCN(CC(=O)NCCCCCNC(=O)COc5ccc(C(=O)Nc6ncc(C)s6)c(C)c5)CC4)cn3)nc2n(C2CCCC2)c1=O] 4-(2-((5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentyl)amino)-2- oxoethoxy)-2-methyl-N-(5-methylthiazol-2-yl)benzamide |
| CPD-037 | 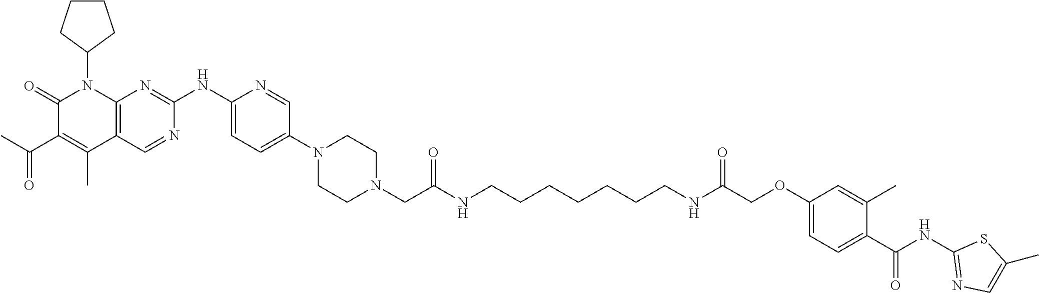 [SMILES: CC(=O)c1c(C)c2cnc(Nc3ccc(N4CCN(CC(=O)NCCCCCCCNC(=O)COc5ccc(C(=O)Nc6ncc(C)s6)c(C)c5)CC4)cn3)nc2n(C2CCCC2)c1=O] 4-(2-((7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptyl)amino)-2- oxoethoxy)-2-methyl-N-(5-methylthiazol-2-yl)benzamide |
| CPD-038 | 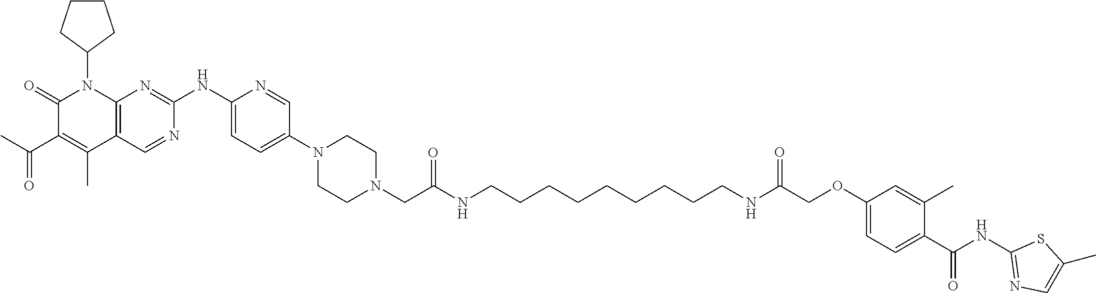 [SMILES: CC(=O)c1c(C)c2cnc(Nc3ccc(N4CCN(CC(=O)NCCCCCCCCCNC(=O)COc5ccc(C(=O)Nc6ncc(C)s6)c(C)c5)CC4)cn3)nc2n(C2CCCC2)c1=O] 4-(2-((9-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)nonyl)amino)-2- oxoethoxy)-2-methyl-N-(5-methylthiazol-2-yl)benzamide |
| CPD-039 | 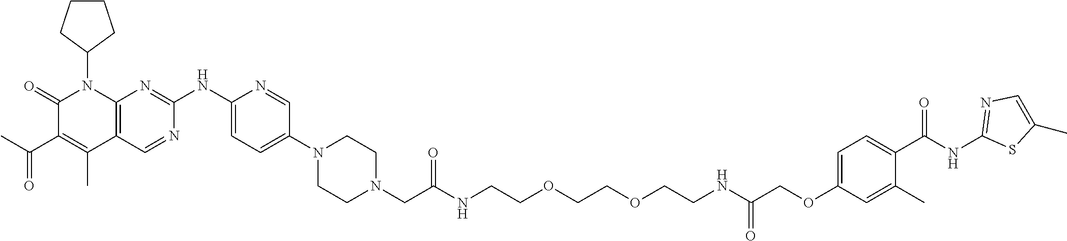 [SMILES: CC(=O)c1c(C)c2cnc(Nc3ccc(N4CCN(CC(=O)NCCOCCOCCNC(=O)COc5ccc(C(=O)Nc6ncc(C)s6)c(C)c5)CC4)cn3)nc2n(C2CCCC2)c1=O] 4-((14-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2,13-dioxo-6,9-dioxa-3,12- diazatetradecyl)oxy)-2-methyl-N-(5-methylthiazol-2-yl)benzamide |
| CPD-040 | 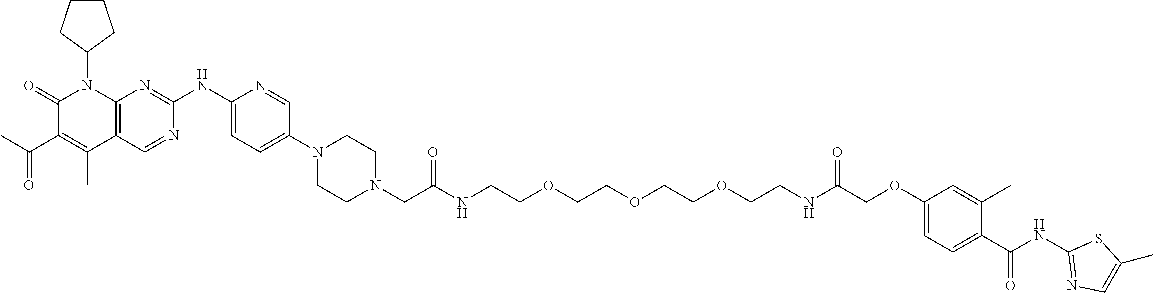 [SMILES: CC(=O)c1c(C)c2cnc(Nc3ccc(N4CCN(CC(=O)NCCOCCOCCOCCNC(=O)COc5ccc(C(=O)Nc6ncc(C)s6)c(C)c5)CC4)cn3)nc2n(C2CCCC2)c1=O] 4-((17-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2,16-dioxo-6,9,12-trioxa-3,15- diazaheptadecyl)oxy)-2-methyl-N-(5-methylthiazol-2-yl)benzamide |
| CPD-041 | 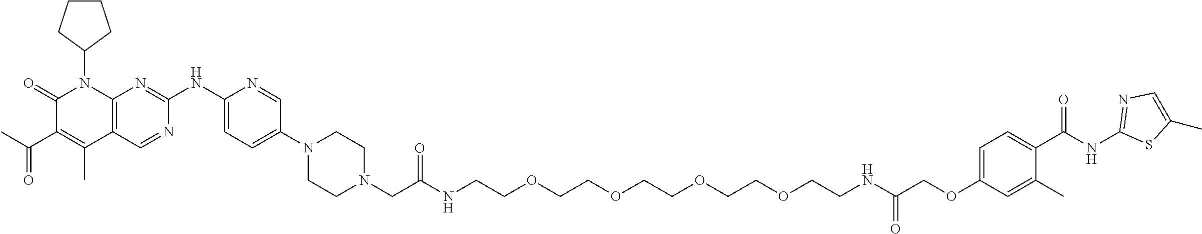 [SMILES: CC(=O)c1c(C)c2cnc(Nc3ccc(N4CCN(CC(=O)NCCOCCOCCOCCOCCNC(=O)COc5ccc(C(=O)Nc6ncc(C)s6)c(C)c5)CC4)cn3)nc2n(C2CCCC2)c1=O] 4-((20-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2,19-dioxo-6,9,12,15-tetraoxa- 3,18-diazaicosyl)oxy)-2-methyl-N-(5-methylthiazol-2-yl)benzamide |
| CPD-042 | 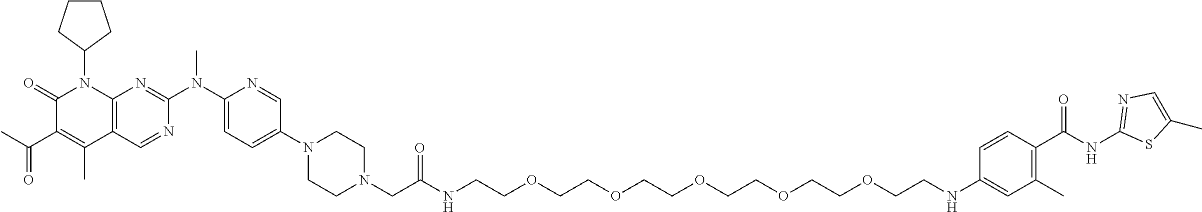 [SMILES: CC(=O)c1c(C)c2cnc(N(C)c3ccc(N4CCN(CC(=O)NCCOCCOCCOCCOCCOCCNc5ccc(C(=O)Nc6ncc(C)s6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)(methyl)amino)pyridin-3-yl)piperazin-1-yl)-2-oxo-6,9,12,15,18- pentaoxa-3-azaicosan-20-yl)amino)-2-methyl-N-(5-methylthiazol-2-yl)benzamide |
| CPD-043 | 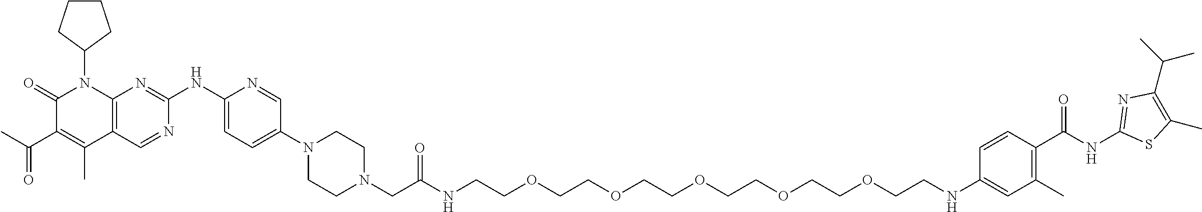 [SMILES: CC(=O)c1c(C)c2cnc(Nc3ccc(N4CCN(CC(=O)NCCOCCOCCOCCOCCOCCNc5ccc(C(=O)Nc6nc(C(C)C)c(C)s6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-N-(4-isopropyl-5-methylthiazol-2-yl)-2-methylbenzamide |
| CPD-044 | 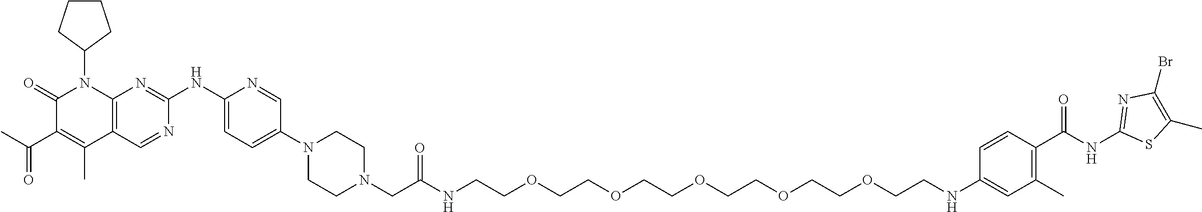 [SMILES: CC(=O)c1c(C)c2cnc(Nc3ccc(N4CCN(CC(=O)NCCOCCOCCOCCOCCOCCNc5ccc(C(=O)Nc6nc(Br)c(C)s6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-N-(4-bromo-5-methylthiazol-2-yl)-2-methylbenzamide |
| CPD-045 | 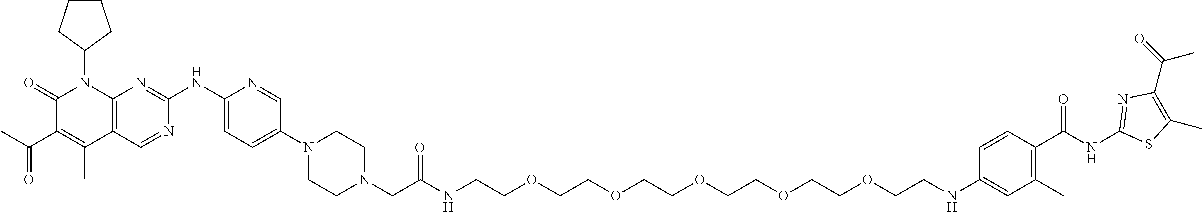 [SMILES: CC(=O)c1nc(NC(=O)c2ccc(NCCOCCOCCOCCOCCOCCNC(=O)CN3CCN(c4ccc(Nc5ncc6c(C)c(C(C)=O)c(=O)n(C7CCCC7)c6n5)nc4)CC3)cc2C)sc1C] N-(4-acetyl-5-methylthiazol-2-yl)-4-((1-(4-(6-((6-acetyl-8-cyclopentyl-5-methyl-7- oxo-7,8-dihydropyrido[2,3-d]pyrimidin-2-yl)amino)pyridin-3-yl)piperazin-1-yl)-2- oxo-6,9,12,15,18-pentaoxa-3-azaicosan-20-yl)amino)-2-methylbenzamide |
| CPD-046 | 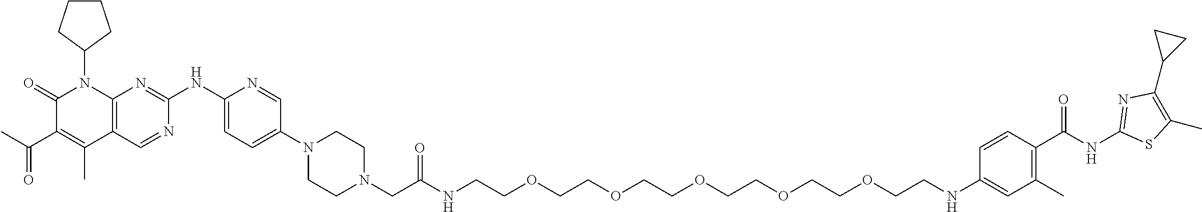 [SMILES: CC(=O)c1c(C)c2cnc(Nc3ccc(N4CCN(CC(=O)NCCOCCOCCOCCOCCOCCNc5ccc(C(=O)Nc6nc(C7CC7)c(C)s6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-N-(4-cyclopropyl-5-methylthiazol-2-yl)-2-methylbenzamide |
| CPD-047 | 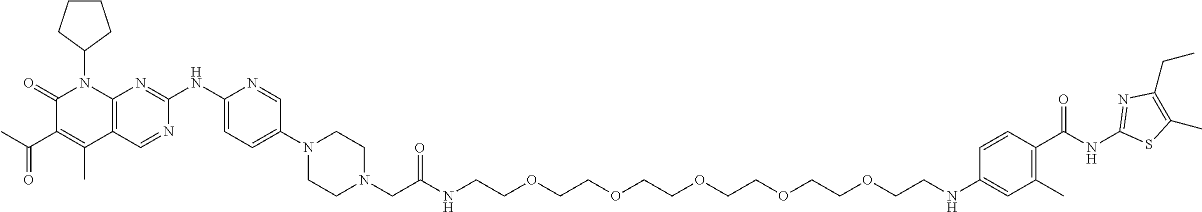 [SMILES: CCc1nc(NC(=O)c2ccc(NCCOCCOCCOCCOCCOCCNC(=O)CN3CCN(c4ccc(Nc5ncc6c(C)c(C(C)=O)c(=O)n(C7CCCC7)c6n5)nc4)CC3)cc2C)sc1C] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-N-(4-ethyl-5-methylthiazol-2-yl)-2-methylbenzamide |
| CPD-048 | 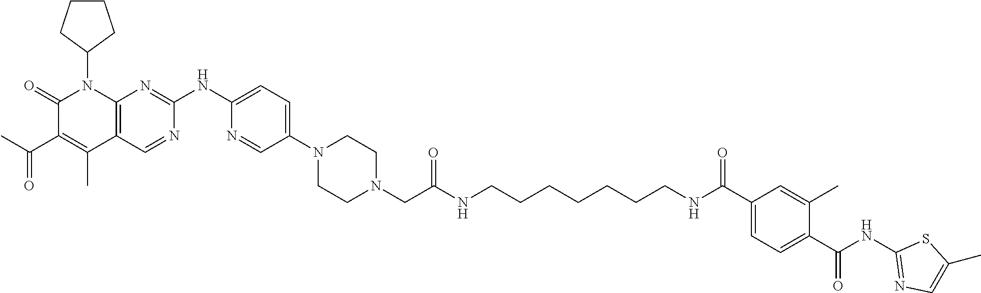 [SMILES: CC(=O)c1c(C)c2cnc(Nc3ccc(N4CCN(CC(=O)NCCCCCCCNC(=O)c5ccc(C(=O)Nc6ncc(C)s6)c(C)c5)CC4)cn3)nc2n(C2CCCC2)c1=O] N⁴-(7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptyl)-2-methyl-N¹- (5-methylthiazol-2-yl)terephthalamide |
| CPD-049 | 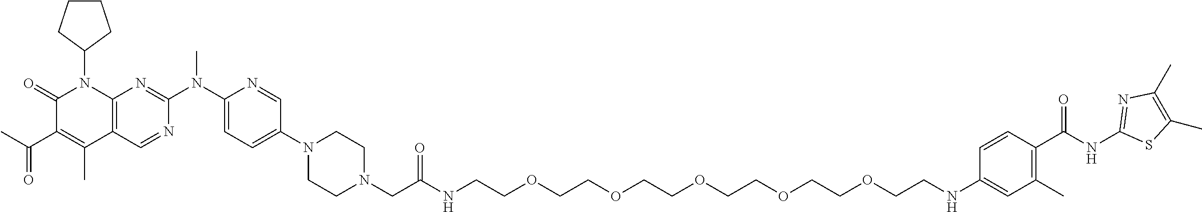 [SMILES: CC(=O)c1c(C)c2cnc(N(C)c3ccc(N4CCN(CC(=O)NCCOCCOCCOCCOCCOCCNc5ccc(C(=O)Nc6nc(C)c(C)s6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)(methyl)amino)pyridin-3-yl)piperazin-1-yl)-2-oxo-6,9,12,15,18- pentaoxa-3-azaicosan-20-yl)amino)-N-(4,5-dimethylthiazol-2-yl)-2-methylbenzamide |
| CPD-050 | 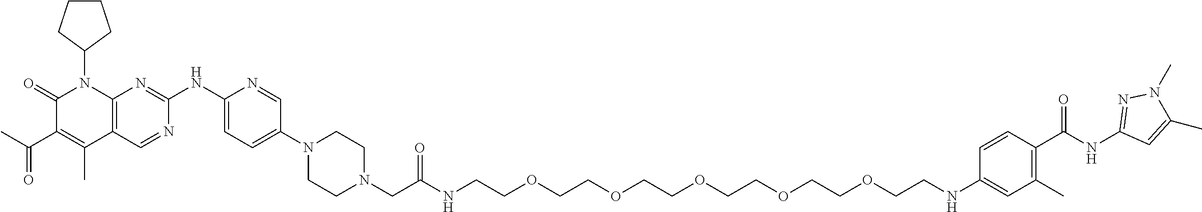 [SMILES: CC(=O)c1c(C)c2cnc(Nc3ccc(N4CCN(CC(=O)NCCOCCOCCOCCOCCOCCNc5ccc(C(=O)Nc6cc(C)n(C)n6)c(C)c5)CC4)cn3)nc2n(C2CCCC2)c1=O] 4-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-N-(1,5-dimethyl-1H-pyrazol-3-yl)-2-methylbenzamide |
| CPD-051 | 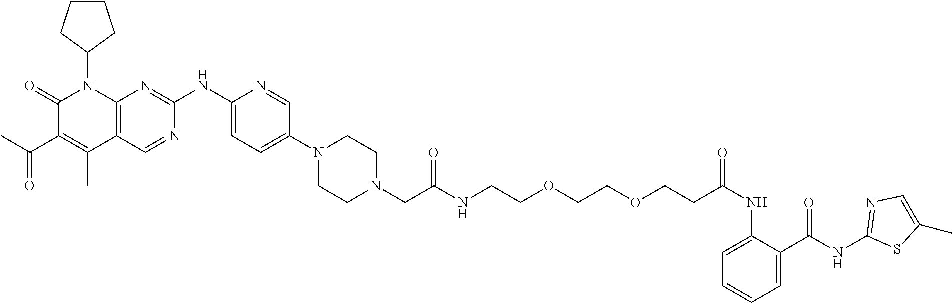 [SMILES: CC(=O)c1c(C)c2cnc(Nc3ccc(N4CCN(CC(=O)NCCOCCOCCC(=O)Nc5ccccc5C(=O)Nc5ncc(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methylthiazol-2-yl)benzamide |
| CPD-052 | 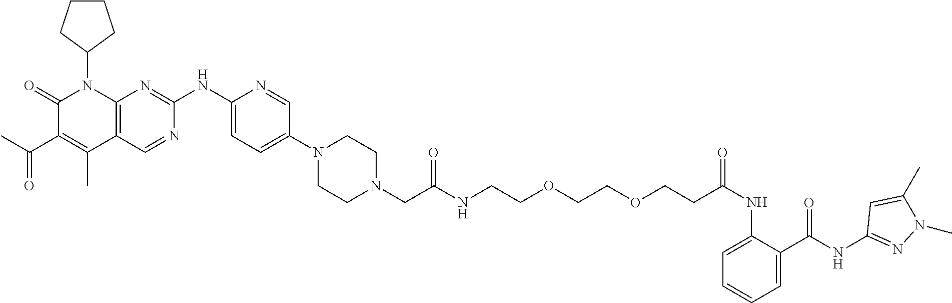 [SMILES: CC(=O)c1c(C)c2cnc(Nc3ccc(N4CCN(CC(=O)NCCOCCOCCC(=O)Nc5ccccc5C(=O)Nc5cc(C)n(C)n5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(1,5-dimethyl-1H-pyrazol-3- yl)benzamide |
| CPD-053 | 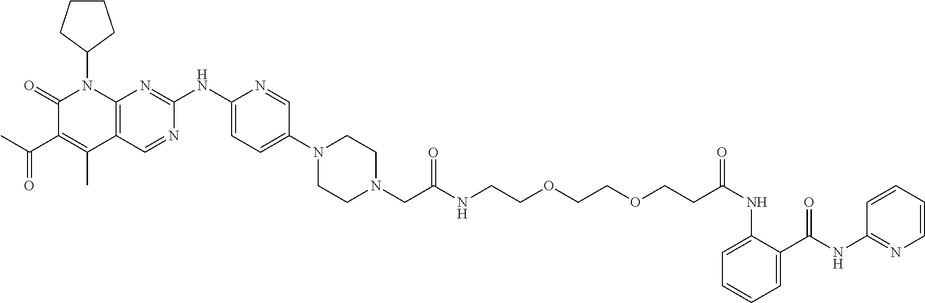 [SMILES: CC(=O)c1c(C)c2cnc(Nc3ccc(N4CCN(CC(=O)NCCOCCOCCC(=O)Nc5ccccc5C(=O)Nc5ccccn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(pyridin-2-yl)benzamide |
| CPD-054 | 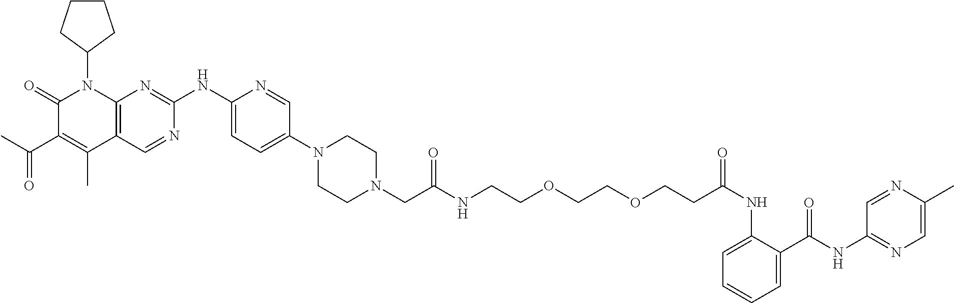 [SMILES: CC(=O)c1c(C)c2cnc(Nc3ccc(N4CCN(CC(=O)NCCOCCOCCC(=O)Nc5ccccc5C(=O)Nc5cnc(C)cn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methylpyrazin-2-yl)benzamide |
| CPD-055 | 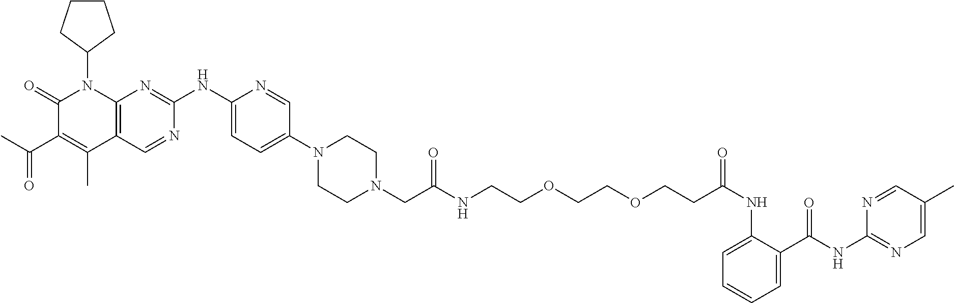 [SMILES: CC(=O)c1c(C)c2cnc(Nc3ccc(N4CCN(CC(=O)NCCOCCOCCC(=O)Nc5ccccc5C(=O)Nc5ncc(C)cn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methylpyrimidin-2-yl)benzamide |
| CPD-056 | 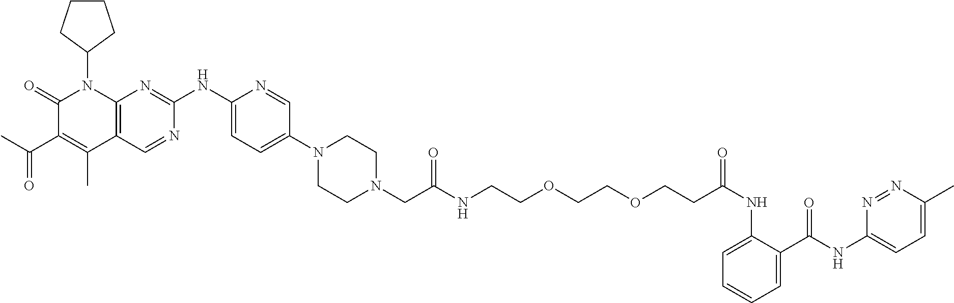 [SMILES: CC(=O)c1c(C)c2cnc(Nc3ccc(N4CCN(CC(=O)NCCOCCOCCC(=O)Nc5ccccc5C(=O)Nc5ccc(C)nn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(6-methylpyridazin-3-yl)benzamide |
| CPD-057 | 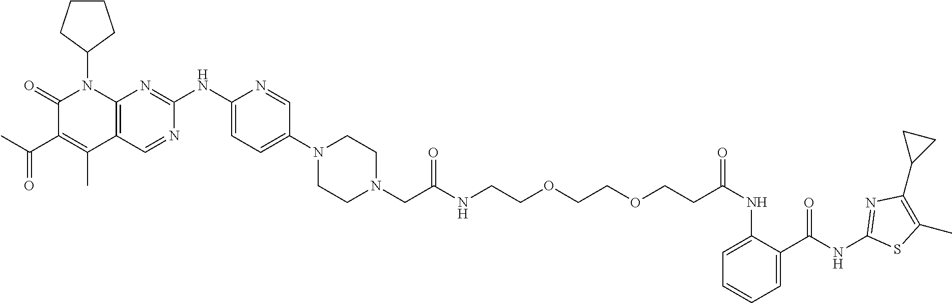 [SMILES: CC(=O)c1c(C)c2cnc(Nc3ccc(N4CCN(CC(=O)NCCOCCOCCC(=O)Nc5ccccc5C(=O)Nc5nc(C6CC6)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(4-cyclopropyl-5-methylthiazol-2- yl)benzamide |
| CPD-058 | 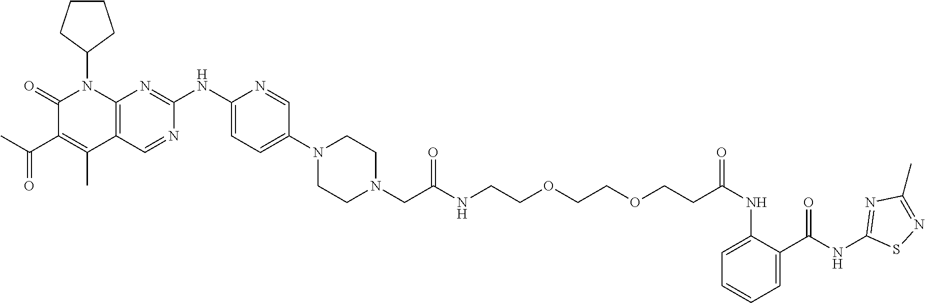 [SMILES: CC(=O)c1c(C)c2cnc(Nc3ccc(N4CCN(CC(=O)NCCOCCOCCC(=O)Nc5ccccc5C(=O)Nc5nc(C)ns5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(3-methyl-1,2,4-thiadiazol-5- yl)benzamide |
| CPD-059 | 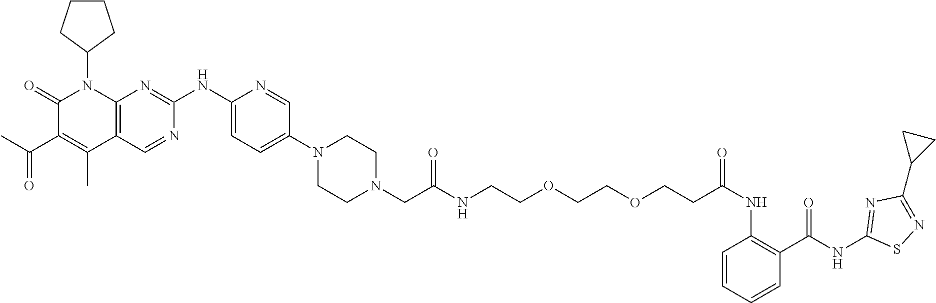 [SMILES: CC(=O)c1c(C)c2cnc(Nc3ccc(N4CCN(CC(=O)NCCOCCOCCC(=O)Nc5ccccc5C(=O)Nc5nc(C6CC6)ns5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(3-cyclopropyl-1,2,4-thiadiazol-5- yl)benzamide |
| CPD-060 | 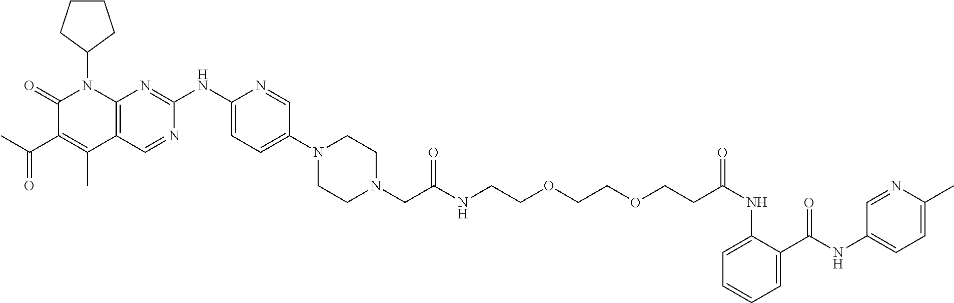 [SMILES: CC(=O)c1c(C)c2cnc(Nc3ccc(N4CCN(CC(=O)NCCOCCOCCC(=O)Nc5ccccc5C(=O)Nc5ccc(C)nc5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(6-methylpyridin-3-yl)benzamide |
| CPD-061 | 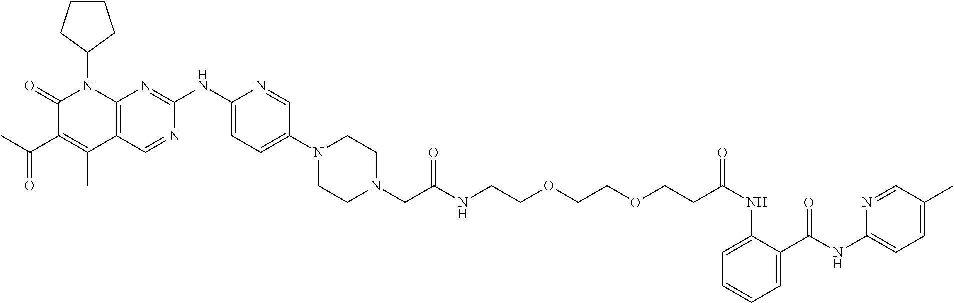 [SMILES: CC(=O)c1c(C)c2cnc(Nc3ccc(N4CCN(CC(=O)NCCOCCOCCC(=O)Nc5ccccc5C(=O)Nc5ccc(C)cn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methylpyridin-2-yl)benzamide |
| CPD-062 | 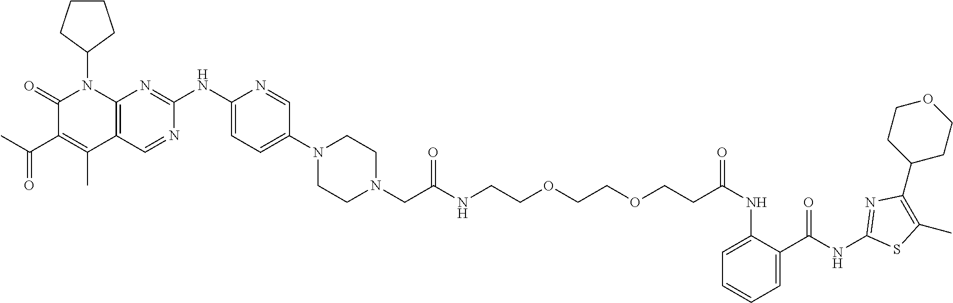 [SMILES: CC(=O)c1c(C)c2cnc(Nc3ccc(N4CCN(CC(=O)NCCOCCOCCC(=O)Nc5ccccc5C(=O)Nc5nc(C6CCOCC6)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methyl-4-(tetrahydro-2H-pyran-4- yl)thiazol-2-yl)benzamide |
| CPD-063 | 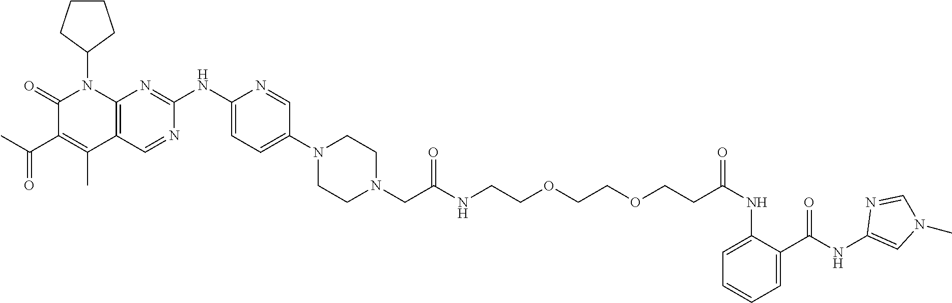 [SMILES: CC(=O)c1c(C)c2cnc(Nc3ccc(N4CCN(CC(=O)NCCOCCOCCC(=O)Nc5ccccc5C(=O)Nc5cn(C)cn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(1-methyl-1H-imidazol-4-yl)benzamide |
| CPD-064 | 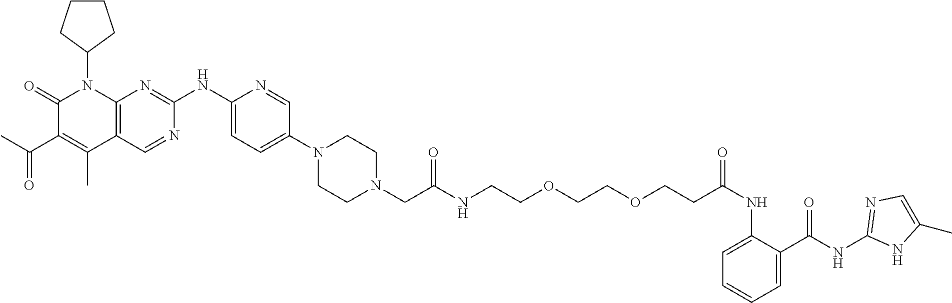 [SMILES: CC(=O)c1c(C)c2cnc(Nc3ccc(N4CCN(CC(=O)NCCOCCOCCC(=O)Nc5ccccc5C(=O)Nc5ncc(C)[nH]5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methyl-1H-imidazol-2-yl)benzamide |
| CPD-065 | 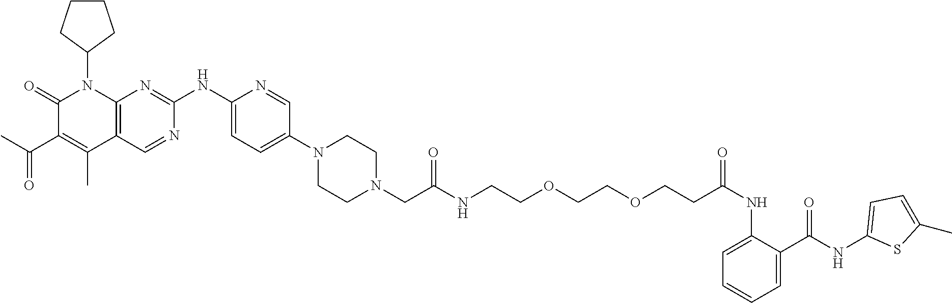 [SMILES: CC(=O)c1c(C)c2cnc(Nc3ccc(N4CCN(CC(=O)NCCOCCOCCC(=O)Nc5ccccc5C(=O)Nc5ccc(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methylthiophen-2-yl)benzamide |
| CPD-066 | 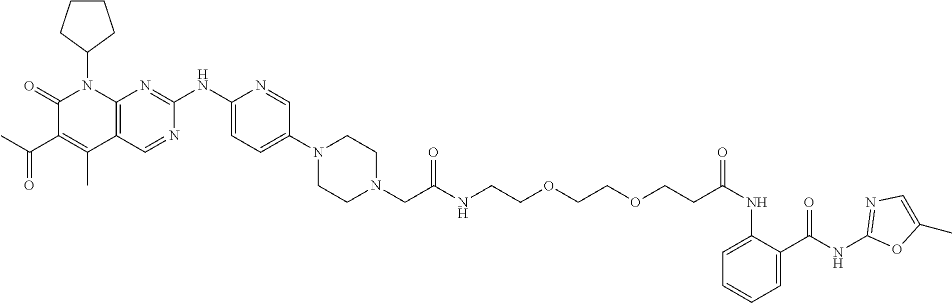 [SMILES: CC(=O)c1c(C)c2cnc(Nc3ccc(N4CCN(CC(=O)NCCOCCOCCC(=O)Nc5ccccc5C(=O)Nc5ncc(C)o5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methyloxazol-2-yl)benzamide |
| CPD-067 | 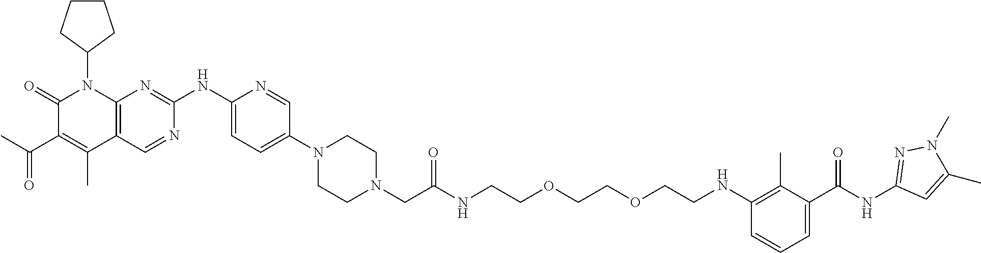 [SMILES: CC(=O)c1c(C)c2cnc(Nc3ccc(N4CCN(CC(=O)NCCOCCOCCNc5cccc(C(=O)Nc6cc(C)n(C)n6)c5C)CC4)cn3)nc2n(C2CCCC2)c1=O] 3-((2-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)ethyl)amino)-N-(1,5-dimethyl-1H-pyrazol-3-yl)-2- methylbenzamide |
| CPD-068 | 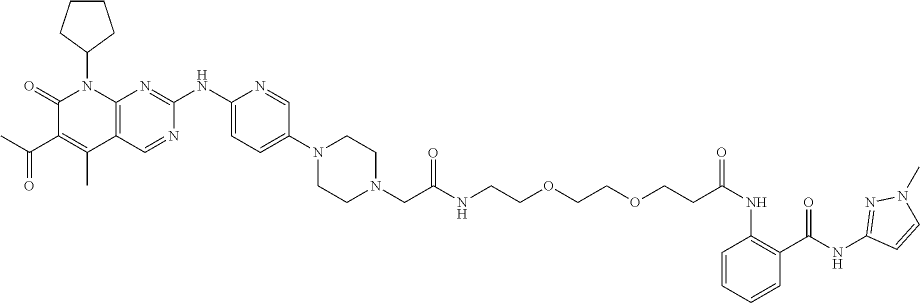 [SMILES: CC(=O)c1c(C)c2cnc(Nc3ccc(N4CCN(CC(=O)NCCOCCOCCC(=O)Nc5ccccc5C(=O)Nc5ccn(C)n5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(1-methyl-1H-pyrazol-3-yl)benzamide |
| CPD-069 | 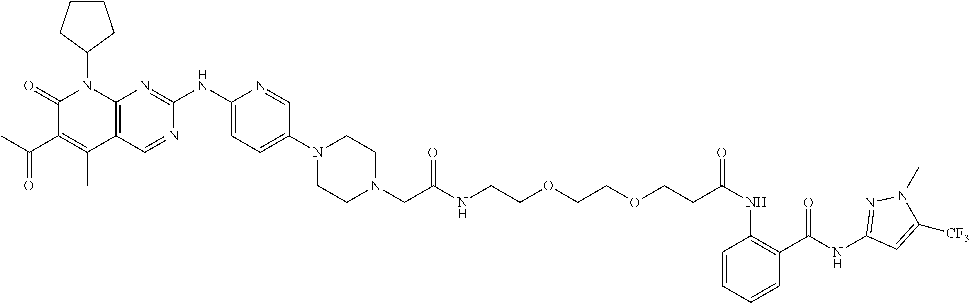 [SMILES: CC(=O)c1c(C)c2cnc(Nc3ccc(N4CCN(CC(=O)NCCOCCOCCC(=O)Nc5ccccc5C(=O)Nc5cc(C(F)(F)F)n(C)n5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(1-methyl-5-(trifluoromethyl)-1H- pyrazol-3-yl)benzamide |
| CPD-070 | 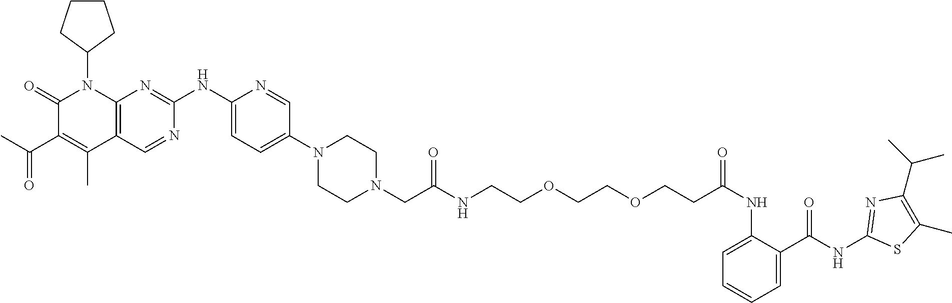 [SMILES: CC(=O)c1c(C)c2cnc(Nc3ccc(N4CCN(CC(=O)NCCOCCOCCC(=O)Nc5ccccc5C(=O)Nc5nc(C(C)C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(4-isopropyl-5-methylthiazol-2- yl)benzamide |
| CPD-071 | 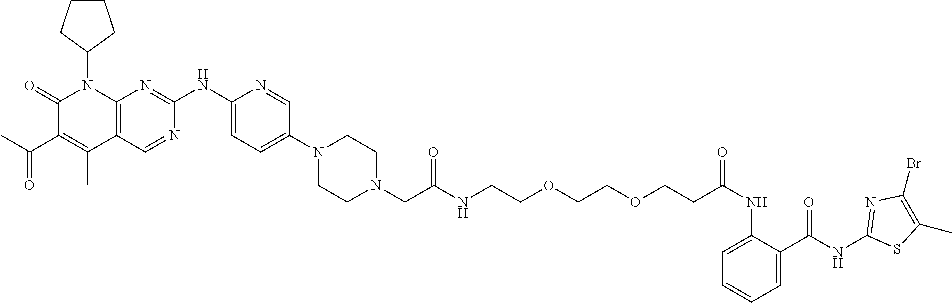 [SMILES: CC(=O)c1c(C)c2cnc(Nc3ccc(N4CCN(CC(=O)NCCOCCOCCC(=O)Nc5ccccc5C(=O)Nc5nc(Br)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(4-bromo-5-methylthiazol-2- yl)benzamide |
| CPD-072 | 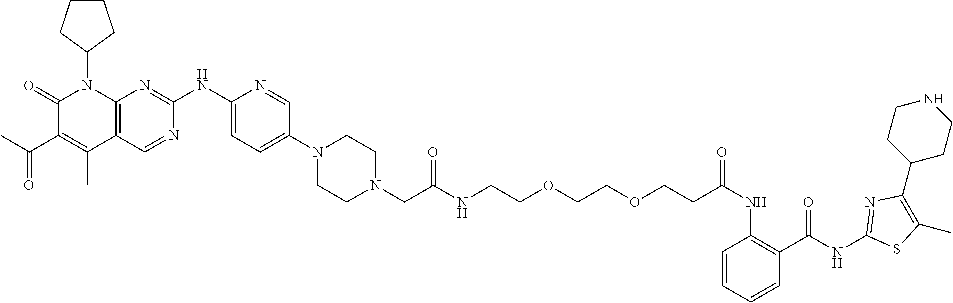 [SMILES: CC(=O)c1c(C)c2cnc(Nc3ccc(N4CCN(CC(=O)NCCOCCOCCC(=O)Nc5ccccc5C(=O)Nc5nc(C6CCNCC6)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methyl-4-(piperidin-4-yl)thiazol-2- yl)benzamide |
| CPD-073 | 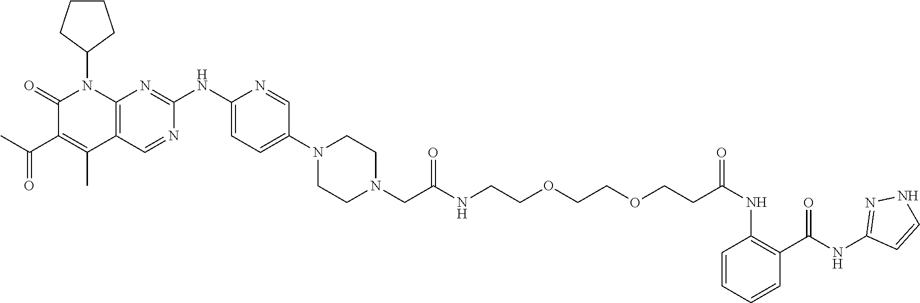 [SMILES: CC(=O)c1c(C)c2cnc(Nc3ccc(N4CCN(CC(=O)NCCOCCOCCC(=O)Nc5ccccc5C(=O)Nc5cc[nH]n5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(1H-pyrazol-3-yl)benzamide |
| CPD-074 | 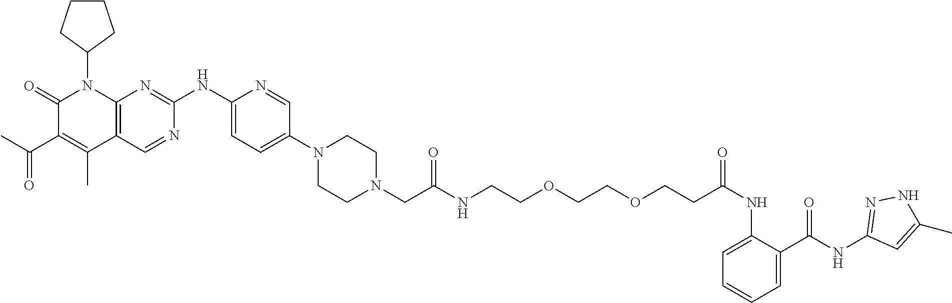 [SMILES: CC(=O)c1c(C)c2cnc(Nc3ccc(N4CCN(CC(=O)NCCOCCOCCC(=O)Nc5ccccc5C(=O)Nc5cc(C)[nH]n5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methyl-1H-pyrazol-3-yl)benzamide |
| CPD-075 | 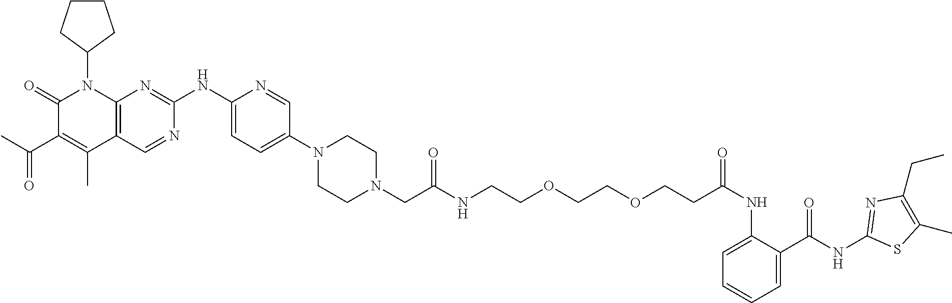 [SMILES: CCc1nc(NC(=O)c2ccccc2NC(=O)CCOCCOCCNC(=O)CN2CCN(c3ccc(Nc4ncc5c(C)c(C(C)=O)c(=O)n(C6CCCC6)c5n4)nc3)CC2)sc1C] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(4-ethyl-5-methylthiazol-2- yl)benzamide |
| CPD-076 | 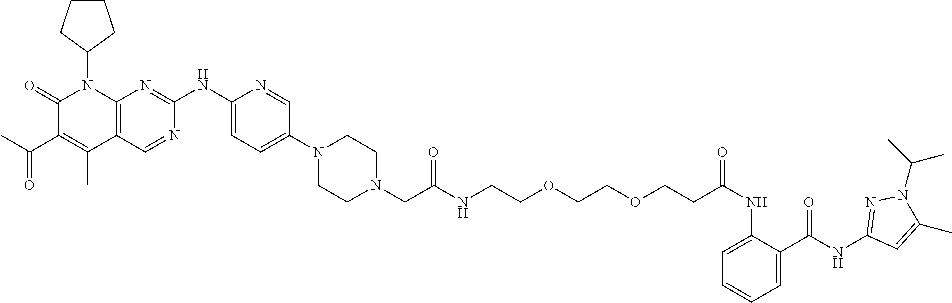 [SMILES: CC(=O)c1c(C)c2cnc(Nc3ccc(N4CCN(CC(=O)NCCOCCOCCC(=O)Nc5ccccc5C(=O)Nc5cc(C)n(C(C)C)n5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(1-isopropyl-5-methyl-1H-pyrazol-3- yl)benzamide |
| CPD-077 | 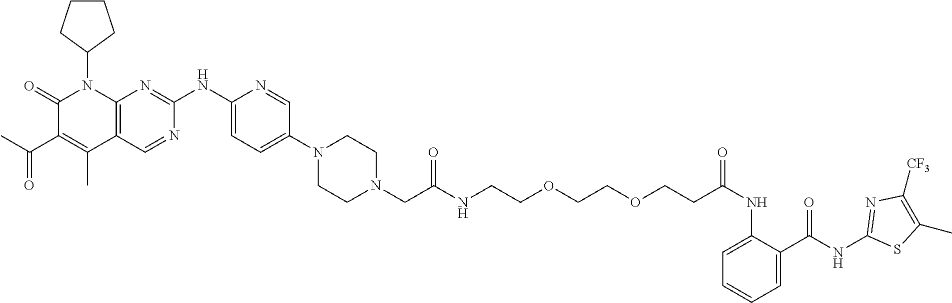 [SMILES: CC(=O)c1c(C)c2cnc(Nc3ccc(N4CCN(CC(=O)NCCOCCOCCC(=O)Nc5ccccc5C(=O)Nc5nc(C(F)(F)F)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methyl-4-(trifluoromethyl)thiazol-2- yl)benzamide |
| CPD-078 | 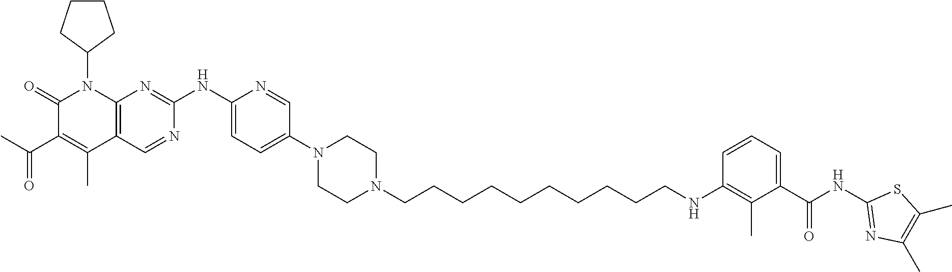 [SMILES: CC(=O)c1c(C)c2cnc(Nc3ccc(N4CCN(CCCCCCCCCCNc5cccc(C(=O)Nc6nc(C)c(C)s6)c5C)CC4)cn3)nc2n(C2CCCC2)c1=O] 3-((10-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)decyl)amino)-N-(4,5- dimethylthiazol-2-yl)-2-methylbenzamide |
| CPD-079 | 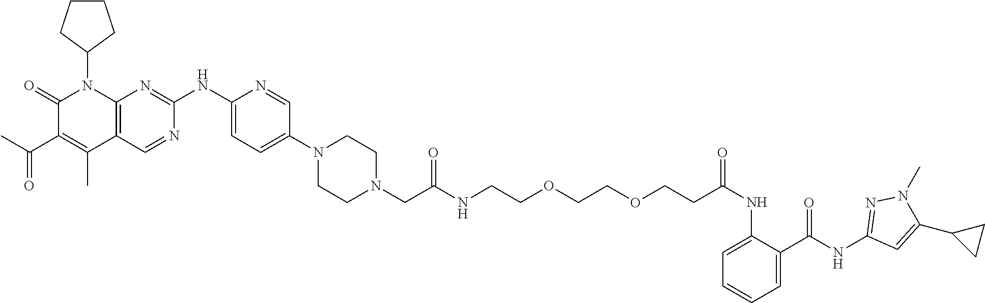 [SMILES: CC(=O)c1c(C)c2cnc(Nc3ccc(N4CCN(CC(=O)NCCOCCOCCC(=O)Nc5ccccc5C(=O)Nc5cc(C6CC6)n(C)n5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-cyclopropyl-1-methyl-1H-pyrazol-3- yl)benzamide |
| CPD-080 | 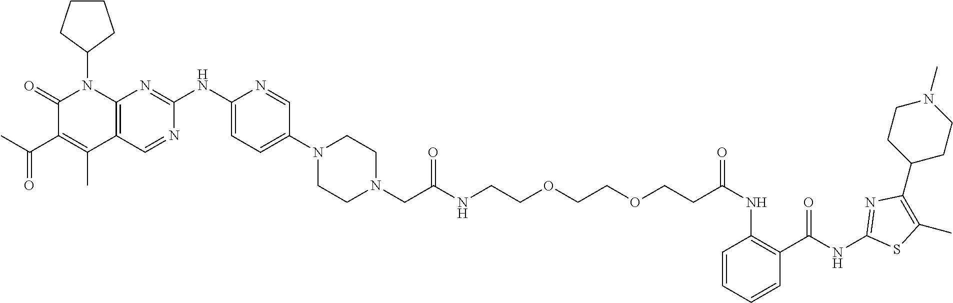 [SMILES: CC(=O)c1c(C)c2cnc(Nc3ccc(N4CCN(CC(=O)NCCOCCOCCC(=O)Nc5ccccc5C(=O)Nc5nc(C6CCN(C)CC6)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methyl-4-(1-methylpiperidin-4- yl)thiazol-2-yl)benzamide |
| CPD-081 | 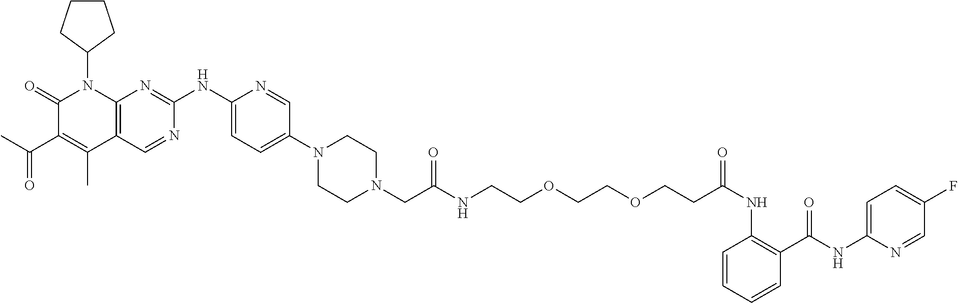 [SMILES: CC(=O)c1c(C)c2cnc(Nc3ccc(N4CCN(CC(=O)NCCOCCOCCC(=O)Nc5ccccc5C(=O)Nc5ccc(F)cn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-fluoropyridin-2-yl)benzamide |
| CPD-082 | 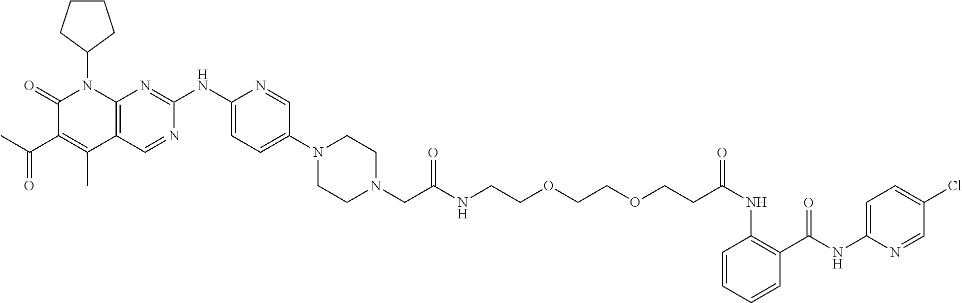 [SMILES: CC(=O)c1c(C)c2cnc(Nc3ccc(N4CCN(CC(=O)NCCOCCOCCC(=O)Nc5ccccc5C(=O)Nc5ccc(Cl)cn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-chloropyridin-2-yl)benzamide |
| CPD-083 | 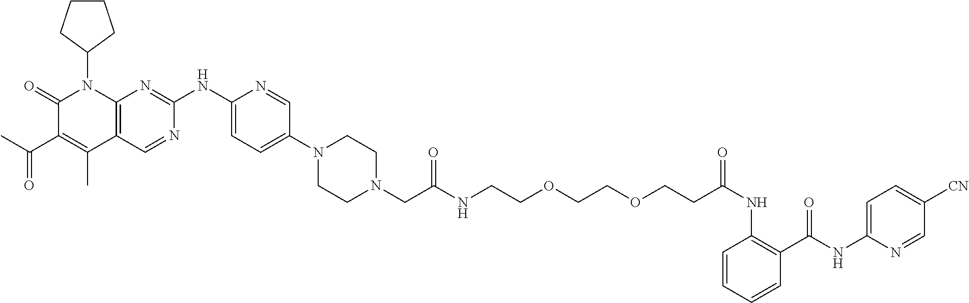 [SMILES: CC(=O)c1c(C)c2cnc(Nc3ccc(N4CCN(CC(=O)NCCOCCOCCC(=O)Nc5ccccc5C(=O)Nc5ccc(C#N)cn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-cyanopyridin-2-yl)benzamide |
| CPD-084 | 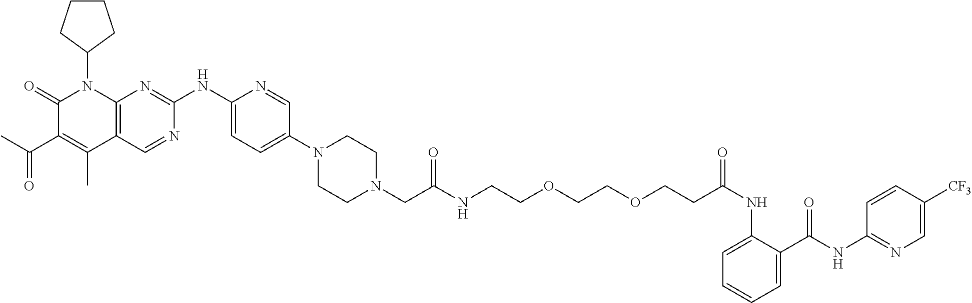 [SMILES: CC(=O)c1c(C)c2cnc(Nc3ccc(N4CCN(CC(=O)NCCOCCOCCC(=O)Nc5ccccc5C(=O)Nc5ccc(C(F)(F)F)cn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-(trifluoromethyl)pyridin-2- yl)benzamide |
| CPD-085 | 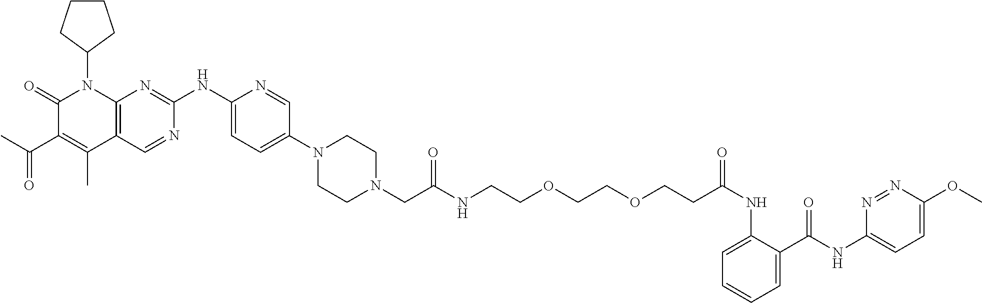 [SMILES: COc1ccc(NC(=O)c2ccccc2NC(=O)CCOCCOCCNC(=O)CN2CCN(c3ccc(Nc4ncc5c(C)c(C(C)=O)c(=O)n(C6CCCC6)c5n4)nc3)CC2)nn1] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(6-methoxypyridazin-3-yl)benzamide |
| CPD-086 | 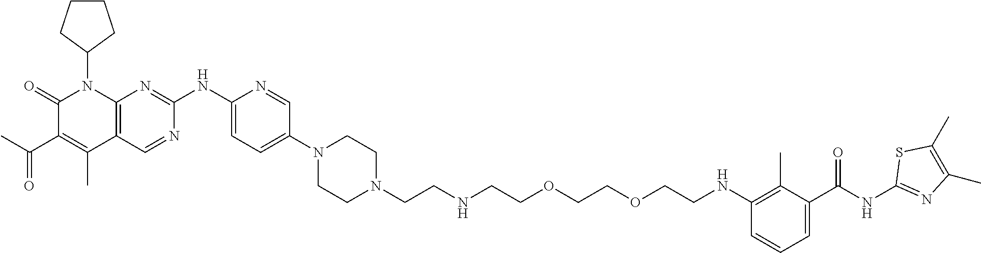 [SMILES: CC(=O)c1c(C)c2cnc(Nc3ccc(N4CCN(CCNCCOCCOCCNc5cccc(C(=O)Nc6nc(C)c(C)s6)c5C)CC4)cn3)nc2n(C2CCCC2)c1=O] 3-((2-(2-(2-((2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)ethyl)amino)ethoxy)ethoxy)ethyl)amino)-N-(4,5-dimethylthiazol-2-yl)-2- methylbenzamide |
| CPD-087 | 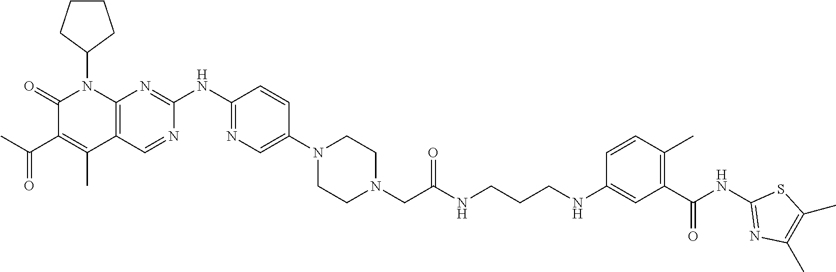 [SMILES: CC(=O)c1c(C)c2cnc(Nc3ccc(N4CCN(CC(=O)NCCCNc5ccc(C)c(C(=O)Nc6nc(C)c(C)s6)c5)CC4)cn3)nc2n(C2CCCC2)c1=O] 5-((3-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)propyl)amino)-N-(4,5- dimethylthiazol-2-yl)-2-methylbenzamide |
| CPD-088 | 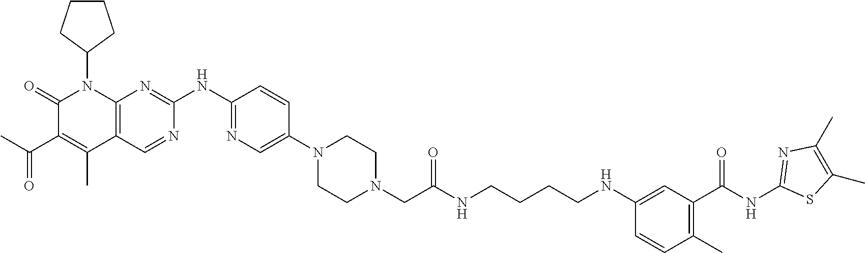 [SMILES: CC(=O)c1c(C)c2cnc(Nc3ccc(N4CCN(CC(=O)NCCCCNc5ccc(C)c(C(=O)Nc6nc(C)c(C)s6)c5)CC4)cn3)nc2n(C2CCCC2)c1=O] 5-((4-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)butyl)amino)-N-(4,5- dimethylthiazol-2-yl)-2-methylbenzamide |
| CPD-089 | 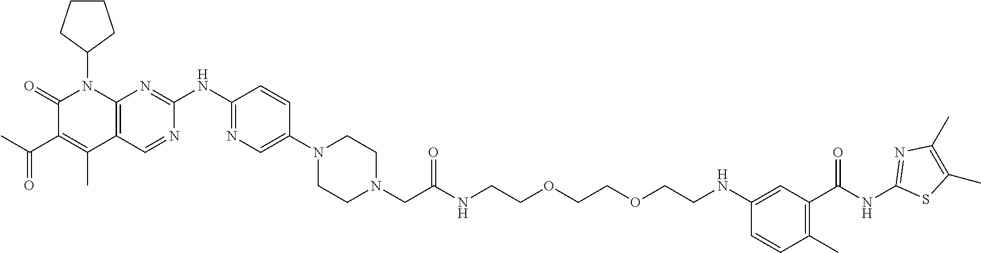 [SMILES: CC(=O)c1c(C)c2cnc(Nc3ccc(N4CCN(CC(=O)NCCOCCOCCNc5ccc(C)c(C(=O)Nc6nc(C)c(C)s6)c5)CC4)cn3)nc2n(C2CCCC2)c1=O] 5-((2-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)ethyl)amino)-N-(4,5-dimethylthiazol-2-yl)-2- methylbenzamide |
| CPD-090 | 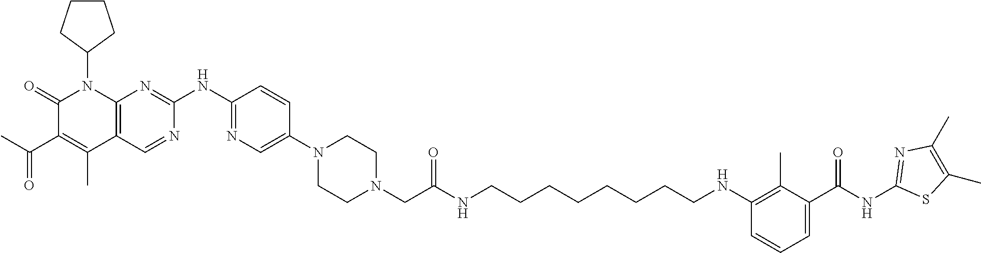 [SMILES: CC(=O)c1c(C)c2cnc(Nc3ccc(N4CCN(CC(=O)NCCCCCCCCNc5cccc(C(=O)Nc6nc(C)c(C)s6)c5C)CC4)cn3)nc2n(C2CCCC2)c1=O] 3-((8-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)octyl)amino)-N-(4,5- dimethylthiazol-2-yl)-2-methylbenzamide |
| CPD-091 | 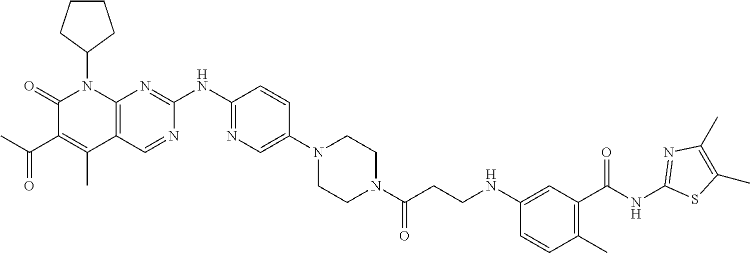 [SMILES: CC(=O)c1c(C)c2cnc(Nc3ccc(N4CCN(C(=O)CCNc5ccc(C)c(C(=O)Nc6nc(C)c(C)s6)c5)CC4)cn3)nc2n(C2CCCC2)c1=O] 5-((3-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-3-oxopropyl)amino)-N-(4,5- dimethylthiazol-2-yl)-2-methylbenzamide |
| CPD-092 | 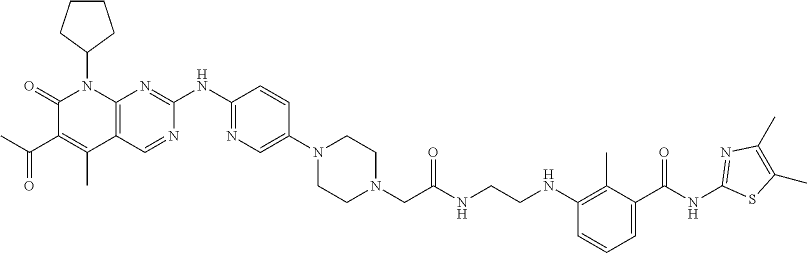 [SMILES: CC(=O)c1c(C)c2cnc(Nc3ccc(N4CCN(CC(=O)NCCNc5cccc(C(=O)Nc6nc(C)c(C)s6)c5C)CC4)cn3)nc2n(C2CCCC2)c1=O] 3-((2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)ethyl)amino)-N-(4,5- dimethylthiazol-2-yl)-2-methylbenzamide |
| CPD-093 | 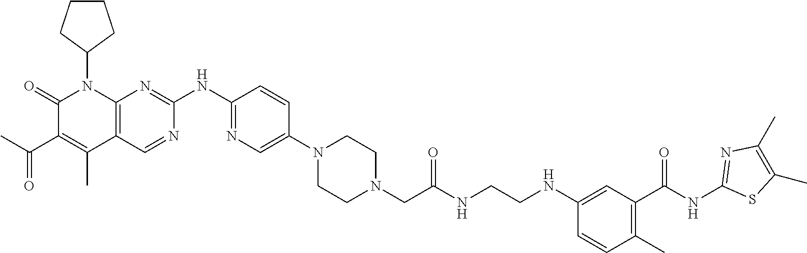 [SMILES: CC(=O)c1c(C)c2cnc(Nc3ccc(N4CCN(CC(=O)NCCNc5ccc(C)c(C(=O)Nc6nc(C)c(C)s6)c5)CC4)cn3)nc2n(C2CCCC2)c1=O] 5-((2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)ethyl)amino)-N-(4,5- dimethylthiazol-2-yl)-2-methylbenzamide |
| CPD-094 | 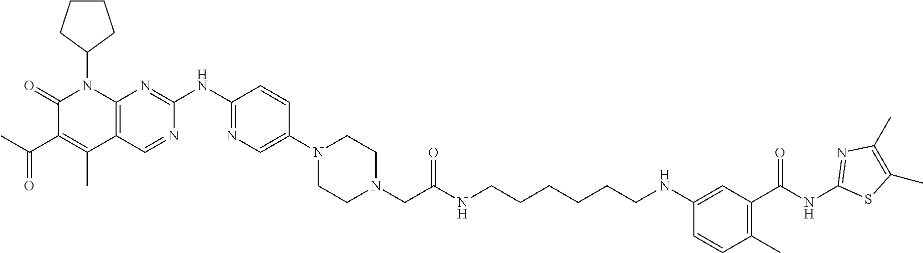 [SMILES: CC(=O)c1c(C)c2cnc(Nc3ccc(N4CCN(CC(=O)NCCCCCCNc5ccc(C)c(C(=O)Nc6nc(C)c(C)s6)c5)CC4)cn3)nc2n(C2CCCC2)c1=O] 5-((6-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)hexyl)amino)-N-(4,5- dimethylthiazol-2-yl)-2-methylbenzamide |
| CPD-095 | 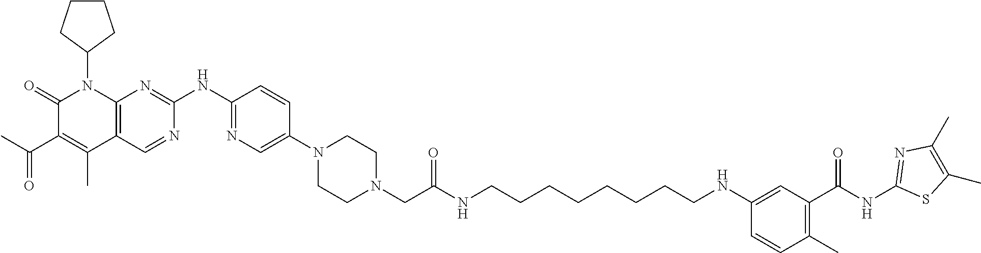 [SMILES: CC(=O)c1c(C)c2cnc(Nc3ccc(N4CCN(CC(=O)NCCCCCCCCNc5ccc(C)c(C(=O)Nc6nc(C)c(C)s6)c5)CC4)cn3)nc2n(C2CCCC2)c1=O] 5-((8-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)octyl)amino)-N-(4,5- dimethylthiazol-2-yl)-2-methylbenzamide |
| CPD-096 | 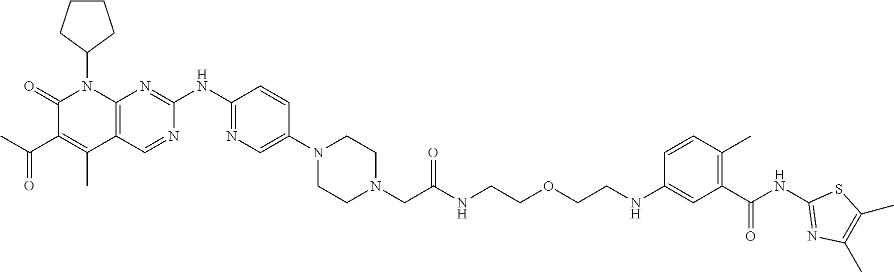 [SMILES: CC(=O)c1c(C)c2cnc(Nc3ccc(N4CCN(CC(=O)NCCOCCNc5ccc(C)c(C(=O)Nc6nc(C)c(C)s6)c5)CC4)cn3)nc2n(C2CCCC2)c1=O] 5-((2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)ethoxy)ethyl)amino)- N-(4,5-dimethylthiazol-2-yl)-2-methylbenzamide |
| CPD-097 | 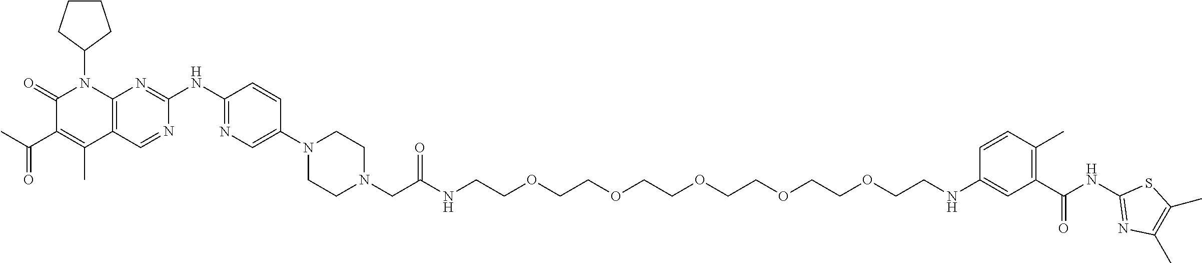 [SMILES: CC(=O)c1c(C)c2cnc(Nc3ccc(N4CCN(CC(=O)NCCOCCOCCOCCOCCOCCNc5ccc(C)c(C(=O)Nc6nc(C)c(C)s6)c5)CC4)cn3)nc2n(C2CCCC2)c1=O] 5-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-N-(4,5-dimethylthiazol-2-yl)-2-methylbenzamide |
| CPD-098 | 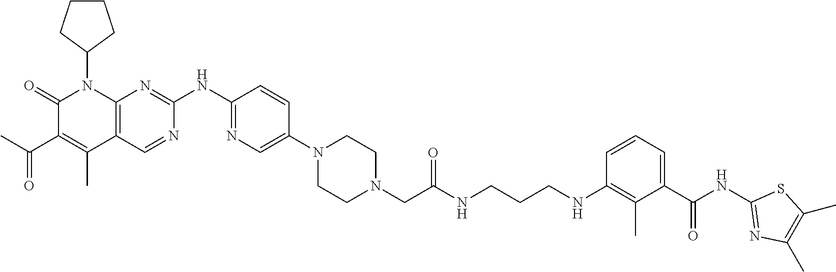 [SMILES: CC(=O)c1c(C)c2cnc(Nc3ccc(N4CCN(CC(=O)NCCCNc5cccc(C(=O)Nc6nc(C)c(C)s6)c5C)CC4)cn3)nc2n(C2CCCC2)c1=O] 3-((3-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)propyl)amino)-N-(4,5- dimethylthiazol-2-yl)-2-methylbenzamide |
| CPD-099 | 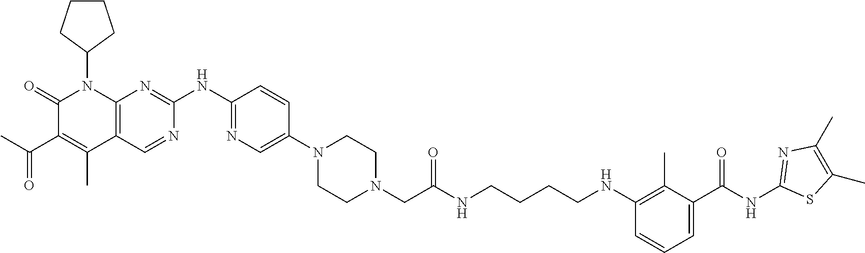 [SMILES: CC(=O)c1c(C)c2cnc(Nc3ccc(N4CCN(CC(=O)NCCCCNc5cccc(C(=O)Nc6nc(C)c(C)s6)c5C)CC4)cn3)nc2n(C2CCCC2)c1=O] 3-((4-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)butyl)amino)-N-(4,5- dimethylthiazol-2-yl)-2-methylbenzamide |
| CPD-100 | 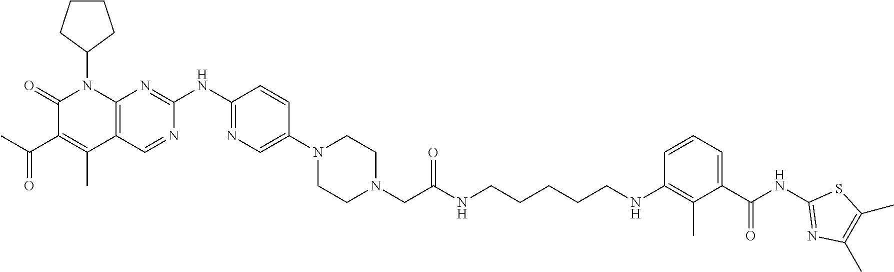 [SMILES: CC(=O)c1c(C)c2cnc(Nc3ccc(N4CCN(CC(=O)NCCCCCNc5cccc(C(=O)Nc6nc(C)c(C)s6)c5C)CC4)cn3)nc2n(C2CCCC2)c1=O] 3-((5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentyl)amino)-N-(4,5- dimethylthiazol-2-yl)-2-methylbenzamide |
| CPD-101 | 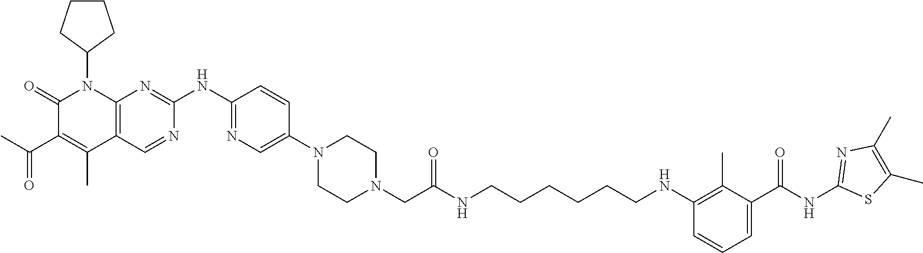 [SMILES: CC(=O)c1c(C)c2cnc(Nc3ccc(N4CCN(CC(=O)NCCCCCCNc5cccc(C(=O)Nc6nc(C)c(C)s6)c5C)CC4)cn3)nc2n(C2CCCC2)c1=O] 3-((6-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)hexyl)amino)-N-(4,5- dimethylthiazol-2-yl)-2-methylbenzamide |
| CPD-102 | 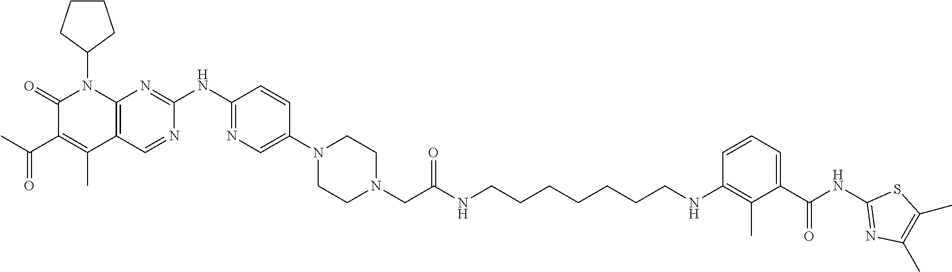 [SMILES: CC(=O)c1c(C)c2cnc(Nc3ccc(N4CCN(CC(=O)NCCCCCCCNc5cccc(C(=O)Nc6nc(C)c(C)s6)c5C)CC4)cn3)nc2n(C2CCCC2)c1=O] 3-((7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptyl)amino)-N-(4,5- dimethylthiazol-2-yl)-2-methylbenzamide |
| CPD-103 | 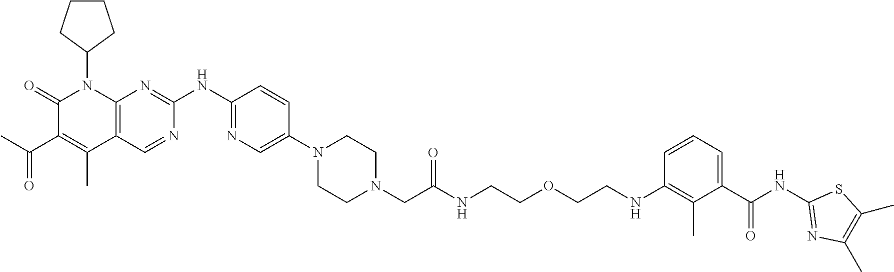 [SMILES: CC(=O)c1c(C)c2cnc(Nc3ccc(N4CCN(CC(=O)NCCOCCNc5cccc(C(=O)Nc6nc(C)c(C)s6)c5C)CC4)cn3)nc2n(C2CCCC2)c1=O] 3-((2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)ethoxy)ethyl)amino)- N-(4,5-dimethylthiazol-2-yl)-2-methylbenzamide |
| CPD-104 | 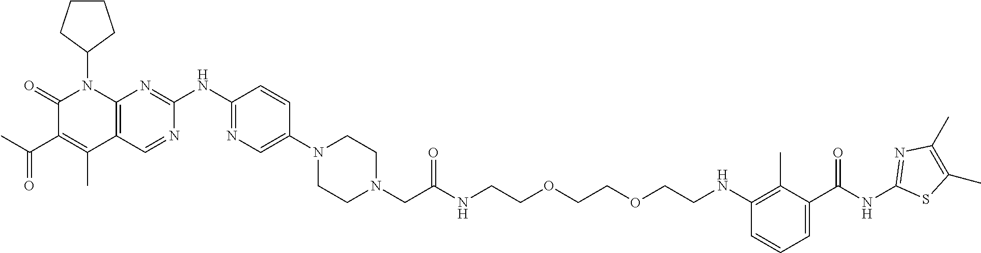 [SMILES: CC(=O)c1c(C)c2cnc(Nc3ccc(N4CCN(CC(=O)NCCOCCOCCNc5cccc(C(=O)Nc6nc(C)c(C)s6)c5C)CC4)cn3)nc2n(C2CCCC2)c1=O] 3-((2-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)ethyl)amino)-N-(4,5-dimethylthiazol-2-yl)-2- methylbenzamide |
| CPD-105 | 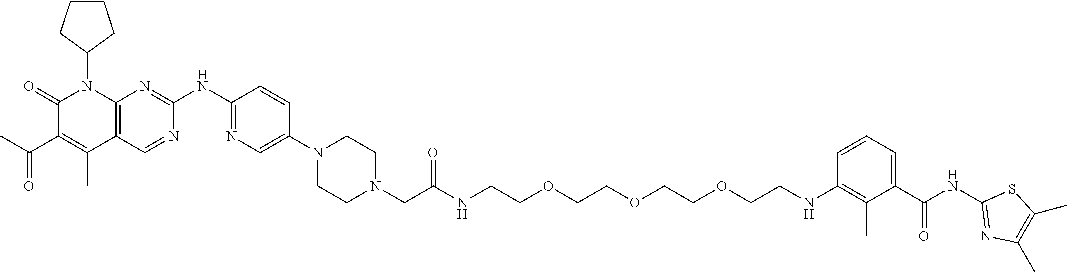 [SMILES: CC(=O)c1c(C)c2cnc(Nc3ccc(N4CCN(CC(=O)NCCOCCOCCOCCNc5cccc(C(=O)Nc6nc(C)c(C)s6)c5C)CC4)cn3)nc2n(C2CCCC2)c1=O] 3-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12-trioxa-3- azatetradecan-14-yl)amino)-N-(4,5-dimethylthiazol-2-yl)-2-methylbenzamide |
| CPD-106 | 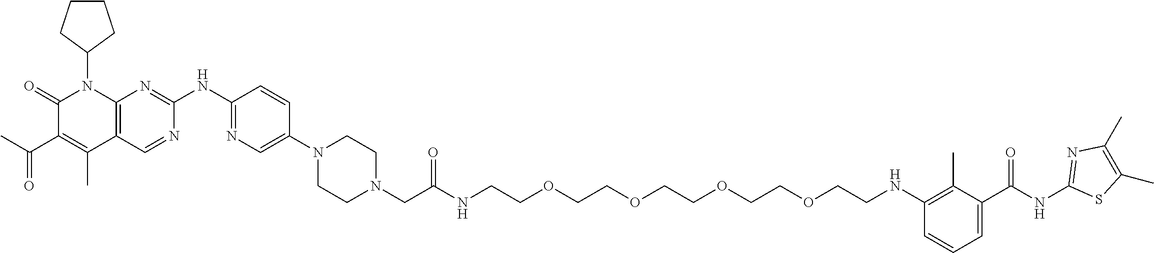 [SMILES: CC(=O)c1c(C)c2cnc(Nc3ccc(N4CCN(CC(=O)NCCOCCOCCOCCOCCNc5cccc(C(=O)Nc6nc(C)c(C)s6)c5C)CC4)cn3)nc2n(C2CCCC2)c1=O] 3-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15-tetraoxa-3- azaheptadecan-17-yl)amino)-N-(4,5-dimethylthiazol-2-yl)-2-methylbenzamide |
| CPD-107 | 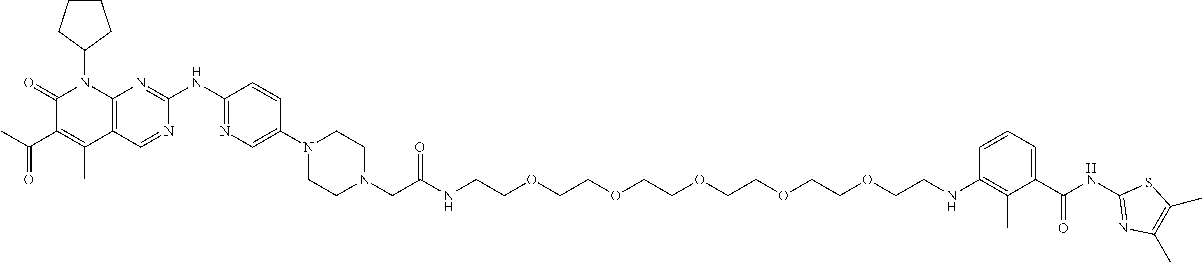 [SMILES: CC(=O)c1c(C)c2cnc(Nc3ccc(N4CCN(CC(=O)NCCOCCOCCOCCOCCOCCNc5cccc(C(=O)Nc6nc(C)c(C)s6)c5C)CC4)cn3)nc2n(C2CCCC2)c1=O] 3-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azaicosan-20-yl)amino)-N-(4,5-dimethylthiazol-2-yl)-2-methylbenzamide |
| CPD-108 | 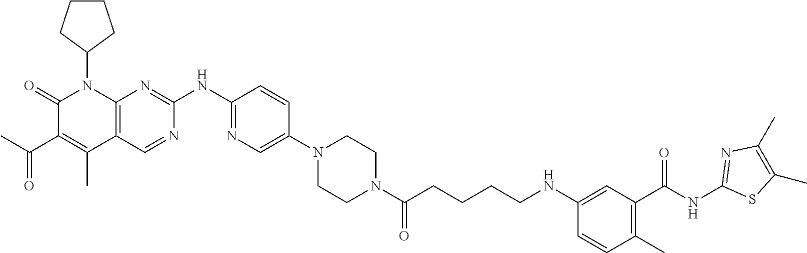 [SMILES: CC(=O)c1c(C)c2cnc(Nc3ccc(N4CCN(C(=O)CCCCNc5ccc(C)c(C(=O)Nc6nc(C)c(C)s6)c5)CC4)cn3)nc2n(C2CCCC2)c1=O] 5-((5-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-5-oxopentyl)amino)-N-(4,5- dimethylthiazol-2-yl)-2-methylbenzamide |
| CPD-109 | 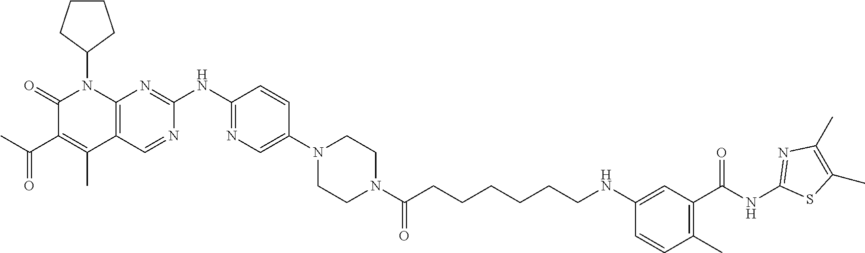 [SMILES: CC(=O)c1c(C)c2cnc(Nc3ccc(N4CCN(C(=O)CCCCCCNc5ccc(C)c(C(=O)Nc6nc(C)c(C)s6)c5)CC4)cn3)nc2n(C2CCCC2)c1=O] 5-((7-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-7-oxoheptyl)amino)-N-(4,5- dimethylthiazol-2-yl)-2-methylbenzamide |
| CPD-110 | 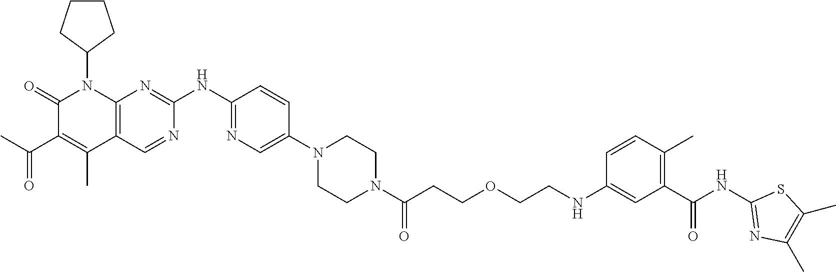 [SMILES: CC(=O)c1c(C)c2cnc(Nc3ccc(N4CCN(C(=O)CCOCCNc5ccc(C)c(C(=O)Nc6nc(C)c(C)s6)c5)CC4)cn3)nc2n(C2CCCC2)c1=O] 5-((2-(3-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-3-oxopropoxy)ethyl)amino)-N- (4,5-dimethylthiazol-2-yl)-2-methylbenzamide |
| CPD-111 | 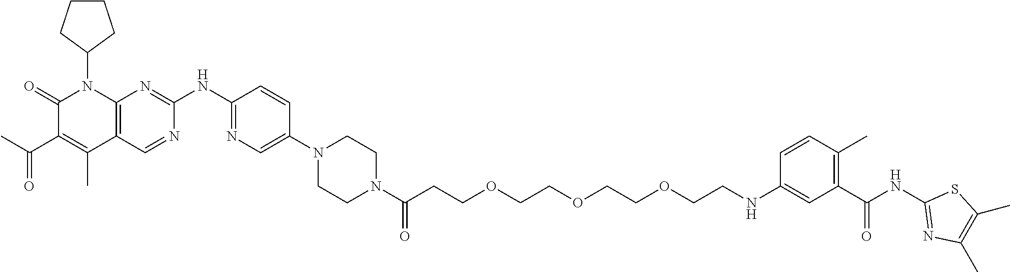 [SMILES: CC(=O)c1c(C)c2cnc(Nc3ccc(N4CCN(C(=O)CCOCCOCCOCCNc5ccc(C)c(C(=O)Nc6nc(C)c(C)s6)c5)CC4)cn3)nc2n(C2CCCC2)c1=O] 5-((2-(2-(2-(3-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-3- oxopropoxy)ethoxy)ethoxy)ethyl)amino)-N-(4,5-dimethylthiazol-2-yl)-2- methylbenzamide |
| CPD-112 | 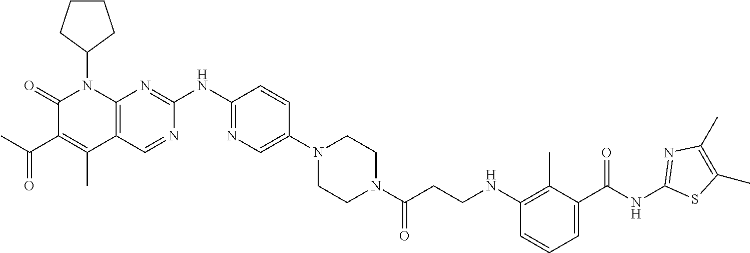 [SMILES: CC(=O)c1c(C)c2cnc(Nc3ccc(N4CCN(C(=O)CCNc5cccc(C(=O)Nc6nc(C)c(C)s6)c5C)CC4)cn3)nc2n(C2CCCC2)c1=O] 3-((3-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-3-oxopropyl)amino)-N-(4,5- dimethylthiazol-2-yl)-2-methylbenzamide |
| CPD-113 | 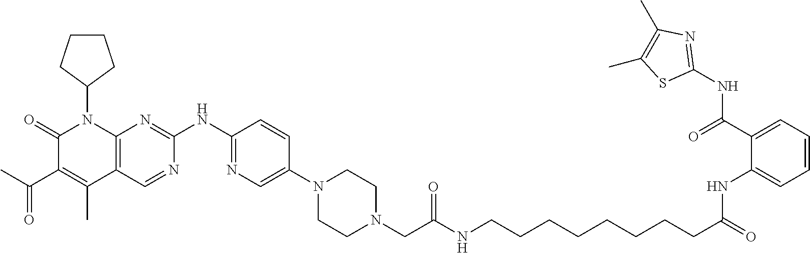 [SMILES: CC(=O)c1c(C)c2cnc(Nc3ccc(N4CCN(CC(=O)NCCCCCCCCC(=O)Nc5ccccc5C(=O)Nc5nc(C)c(C)s5)CC4)cn3)nc2n(C2CCCC2)c1=O] 2-(9-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)nonanamido)-N-(4,5- dimethylthiazol-2-yl)benzamide |
| CPD-114 | 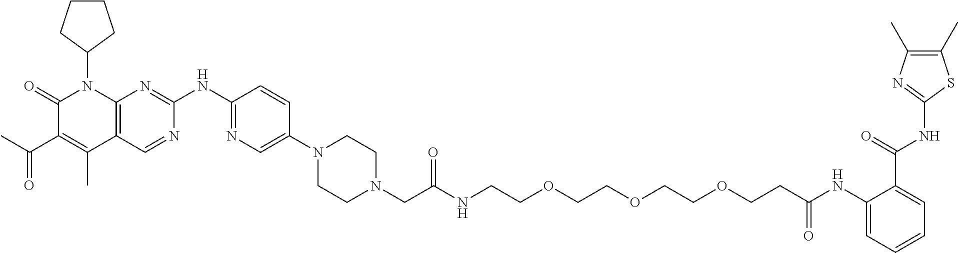 [SMILES: CC(=O)c1c(C)c2cnc(Nc3ccc(N4CCN(CC(=O)NCCOCCOCCOCCC(=O)Nc5ccccc5C(=O)Nc5nc(C)c(C)s5)CC4)cn3)nc2n(C2CCCC2)c1=O] 2-(1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12-trioxa-3- azapentadecan-15-amido)-N-(4,5-dimethylthiazol-2-yl)benzamide |
| CPD-115 | 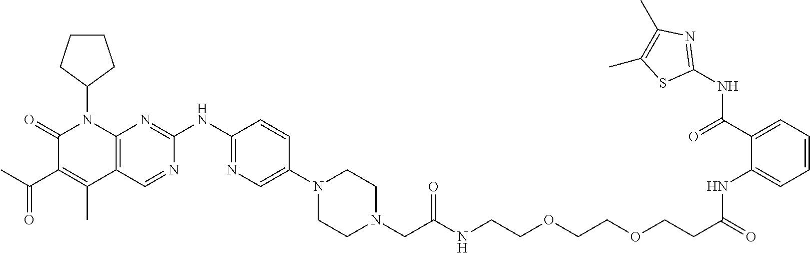 [SMILES: CC(=O)c1c(C)c2cnc(Nc3ccc(N4CCN(CC(=O)NCCOCCOCCC(=O)Nc5ccccc5C(=O)Nc5nc(C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(4,5-dimethylthiazol-2-yl)benzamide |
| CPD-116 | 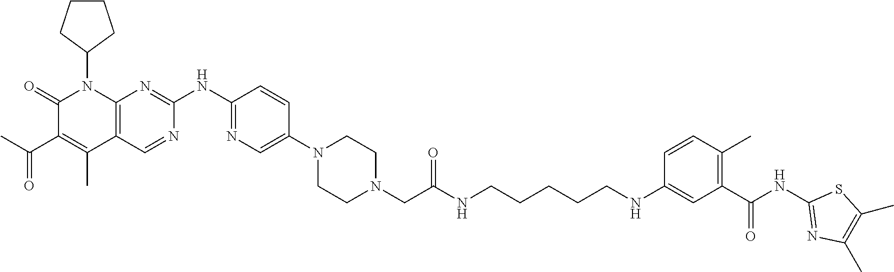 [SMILES: CC(=O)c1c(C)c2cnc(Nc3ccc(N4CCN(CC(=O)NCCCCCNc5ccc(C)c(C(=O)Nc6nc(C)c(C)s6)c5)CC4)cn3)nc2n(C2CCCC2)c1=O] 5-((5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentyl)amino)-N-(4,5- dimethylthiazol-2-yl)-2-methylbenzamide |
| CPD-117 | 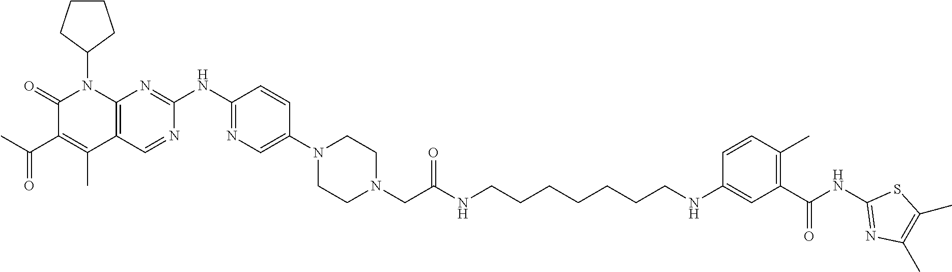 [SMILES: CC(=O)c1c(C)c2cnc(Nc3ccc(N4CCN(CC(=O)NCCCCCCCNc5ccc(C)c(C(=O)Nc6nc(C)c(C)s6)c5)CC4)cn3)nc2n(C2CCCC2)c1=O] 5-((7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptyl)amino)-N-(4,5- dimethylthiazol-2-yl)-2-methylbenzamide |
| CPD-118 | 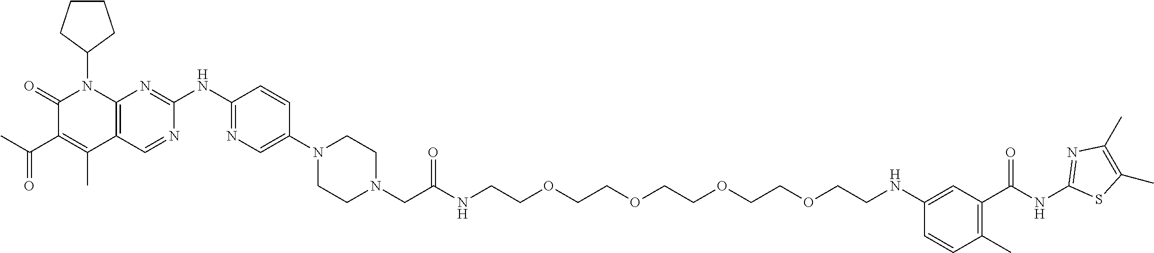 [SMILES: CC(=O)c1c(C)c2cnc(Nc3ccc(N4CCN(CC(=O)NCCOCCOCCOCCOCCNc5ccc(C)c(C(=O)Nc6nc(C)c(C)s6)c5)CC4)cn3)nc2n(C2CCCC2)c1=O] 5-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15-tetraoxa-3- azaheptadecan-17-yl)amino)-N-(4,5-dimethylthiazol-2-yl)-2-methylbenzamide |
| CPD-119 | 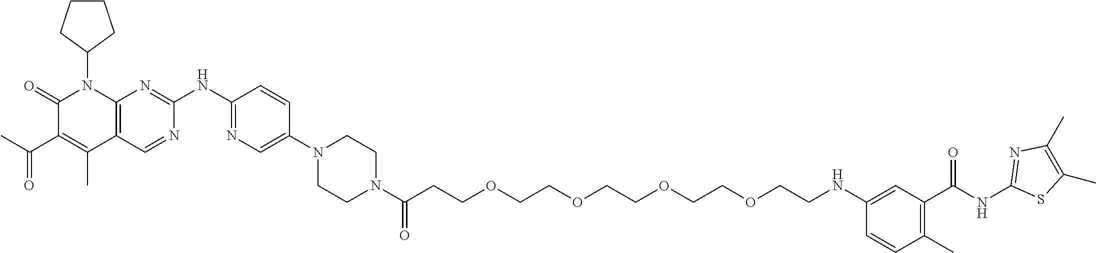 [SMILES: CC(=O)c1c(C)c2cnc(Nc3ccc(N4CCN(C(=O)CCOCCOCCOCCOCCNc5ccc(C)c(C(=O)Nc6nc(C)c(C)s6)c5)CC4)cn3)nc2n(C2CCCC2)c1=O] 5-((15-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-15-oxo-3,6,9,12- tetraoxapentadecyl)amino)-N-(4,5-dimethylthiazol-2-yl)-2-methylbenzamide |
| CPD-120 | 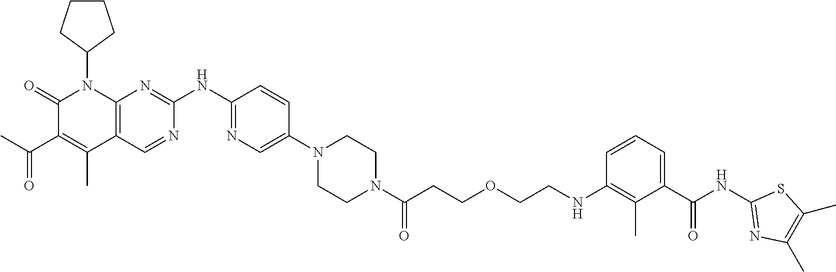 [SMILES: CC(=O)c1c(C)c2cnc(Nc3ccc(N4CCN(C(=O)CCOCCNc5cccc(C(=O)Nc6nc(C)c(C)s6)c5C)CC4)cn3)nc2n(C2CCCC2)c1=O] 3-((2-(3-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-3-oxopropoxy)ethyl)amino)-N- (4,5-dimethylthiazol-2-yl)-2-methylbenzamide |
| CPD-121 | 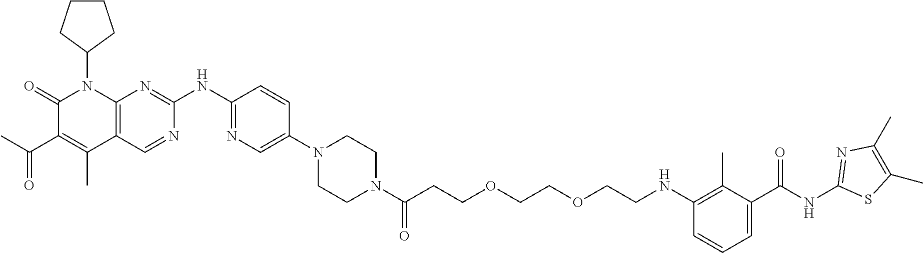 [SMILES: CC(=O)c1c(C)c2cnc(Nc3ccc(N4CCN(C(=O)CCOCCOCCNc5cccc(C(=O)Nc6nc(C)c(C)s6)c5C)CC4)cn3)nc2n(C2CCCC2)c1=O] 3-((2-(2-(3-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-3- oxopropoxy)ethoxy)ethyl)amino)-N-(4,5-dimethylthiazol-2-yl)-2-methylbenzamide |
| CPD-122 | 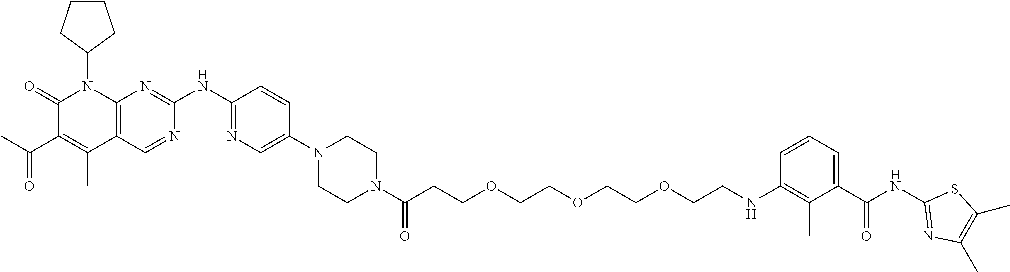 [SMILES: CC(=O)c1c(C)c2cnc(Nc3ccc(N4CCN(C(=O)CCOCCOCCOCCNc5cccc(C(=O)Nc6nc(C)c(C)s6)c5C)CC4)cn3)nc2n(C2CCCC2)c1=O] 3-((2-(2-(2-(3-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-3- oxopropoxy)ethoxy)ethoxy)ethyl)amino)-N-(4,5-dimethylthiazol-2-yl)-2- methylbenzamide |
| CPD-123 | 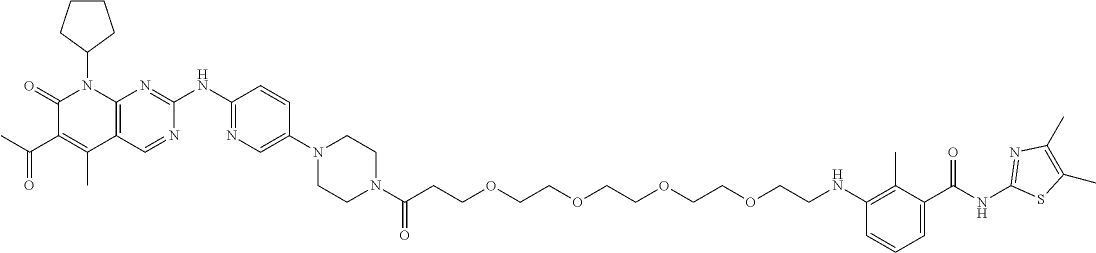 [SMILES: CC(=O)c1c(C)c2cnc(Nc3ccc(N4CCN(C(=O)CCOCCOCCOCCOCCNc5cccc(C(=O)Nc6nc(C)c(C)s6)c5C)CC4)cn3)nc2n(C2CCCC2)c1=O] 3-((15-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-15-oxo-3,6,9,12- tetraoxapentadecyl)amino)-N-(4,5-dimethylthiazol-2-yl)-2-methylbenzamide |
| CPD-124 | 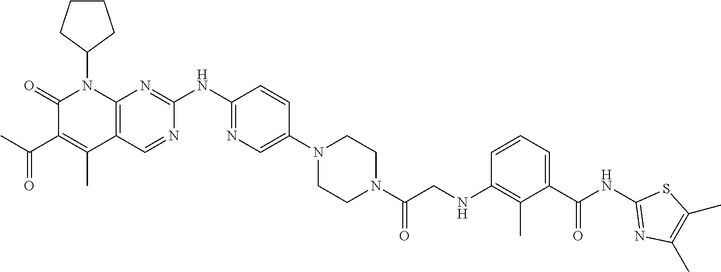 [SMILES: CC(=O)c1c(C)c2cnc(Nc3ccc(N4CCN(C(=O)CNc5cccc(C(=O)Nc6nc(C)c(C)s6)c5C)CC4)cn3)nc2n(C2CCCC2)c1=O] 3-((2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxoethyl)amino)-N-(4,5- dimethylthiazol-2-yl)-2-methylbenzamide |
| CPD-125 | 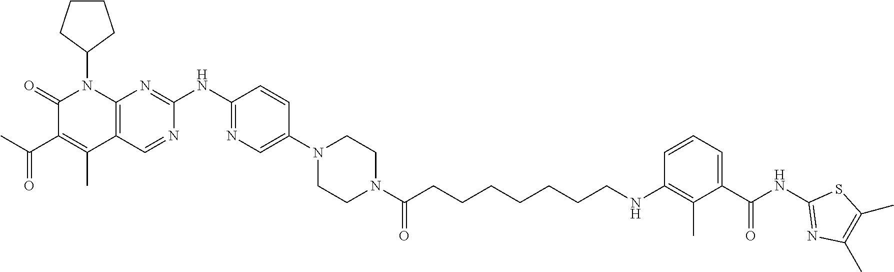 [SMILES: CC(=O)c1c(C)c2cnc(Nc3ccc(N4CCN(C(=O)CCCCCCCNc5cccc(C(=O)Nc6nc(C)c(C)s6)c5C)CC4)cn3)nc2n(C2CCCC2)c1=O] 3-((8-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-8-oxooctyl)amino)-N-(4,5- dimethylthiazol-2-yl)-2-methylbenzamide |
| CPD-126 | 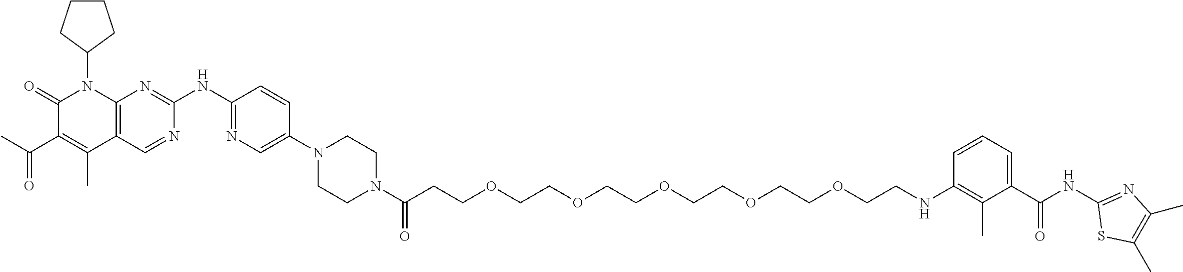 [SMILES: CC(=O)c1c(C)c2cnc(Nc3ccc(N4CCN(C(=O)CCOCCOCCOCCOCCOCCNc5cccc(C(=O)Nc6nc(C)c(C)s6)c5C)CC4)cn3)nc2n(C2CCCC2)c1=O] 3-((18-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-18-oxo-3,6,9,12,15- pentaoxaoctadecyl)amino)-N-(4,5-dimethylthiazol-2-yl)-2-methylbenzamide |
| CPD-127 | 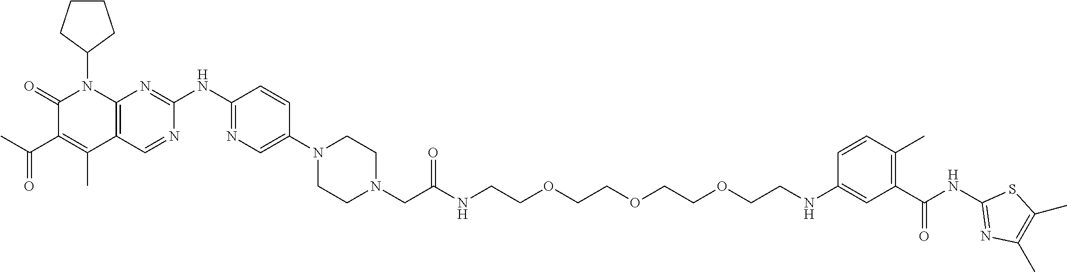 [SMILES: CC(=O)c1c(C)c2cnc(Nc3ccc(N4CCN(CC(=O)NCCOCCOCCOCCNc5ccc(C)c(C(=O)Nc6nc(C)c(C)s6)c5)CC4)cn3)nc2n(C2CCCC2)c1=O] 5-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12-trioxa-3- azatetradecan-14-yl)amino)-N-(4,5-dimethylthiazol-2-yl)-2-methylbenzamide |
| CPD-128 | 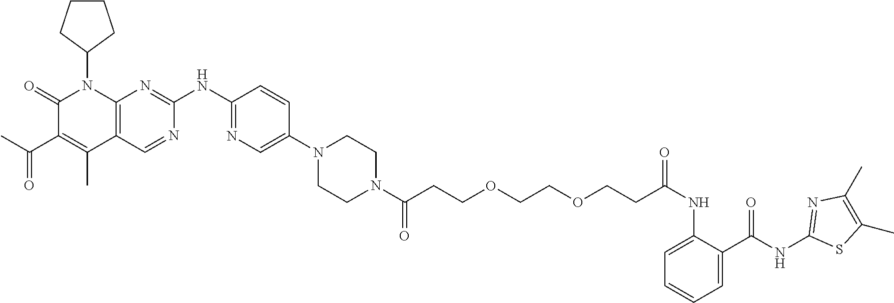 [SMILES: CC(=O)c1c(C)c2cnc(Nc3ccc(N4CCN(C(=O)CCOCCOCCC(=O)Nc5ccccc5C(=O)Nc5nc(C)c(C)s5)CC4)cn3)nc2n(C2CCCC2)c1=O] 2-(3-(2-(3-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-3- oxopropoxy)ethoxy)propanamido)-N-(4,5-dimethylthiazol-2-yl)benzamide |
| CPD-129 | 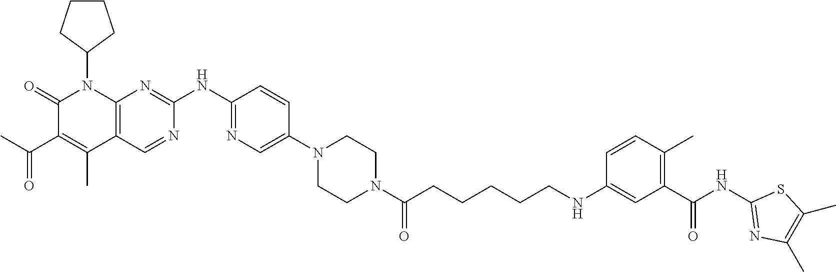 [SMILES: CC(=O)c1c(C)c2cnc(Nc3ccc(N4CCN(C(=O)CCCCCNc5ccc(C)c(C(=O)Nc6nc(C)c(C)s6)c5)CC4)cn3)nc2n(C2CCCC2)c1=O] 5-((6-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-6-oxohexyl)amino)-N-(4,5- dimethylthiazol-2-yl)-2-methylbenzamide |
| CPD-130 | 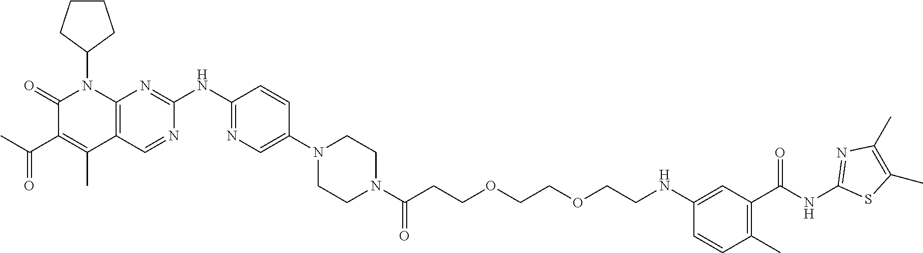 [SMILES: CC(=O)c1c(C)c2cnc(Nc3ccc(N4CCN(C(=O)CCOCCOCCNc5ccc(C)c(C(=O)Nc6nc(C)c(C)s6)c5)CC4)cn3)nc2n(C2CCCC2)c1=O] 5-((2-(2-(3-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-3- oxopropoxy)ethoxy)ethyl)amino)-N-(4,5-dimethylthiazol-2-yl)-2-methylbenzamide |
| CPD-131 | 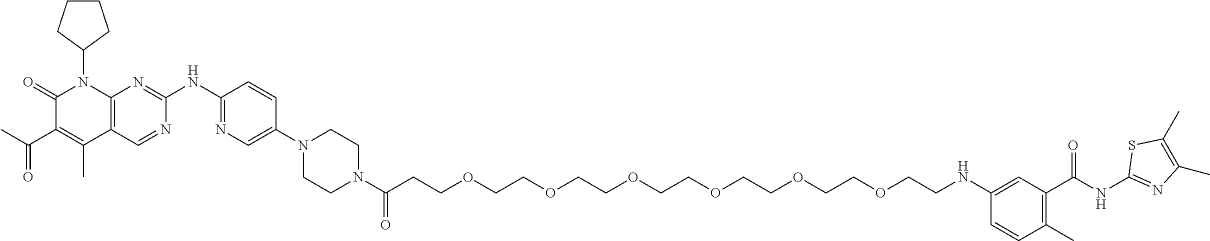 [SMILES: CC(=O)c1c(C)c2cnc(Nc3ccc(N4CCN(C(=O)CCOCCOCCOCCOCCOCCOCCNc5ccc(C)c(C(=O)Nc6nc(C)c(C)s6)c5)CC4)cn3)nc2n(C2CCCC2)c1=O] 5-((21-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-21-oxo-3,6,9,12,15,18- hexaoxahenicosyl)amino)-N-(4,5-dimethylthiazol-2-yl)-2-methylbenzamide |
| CPD-132 | 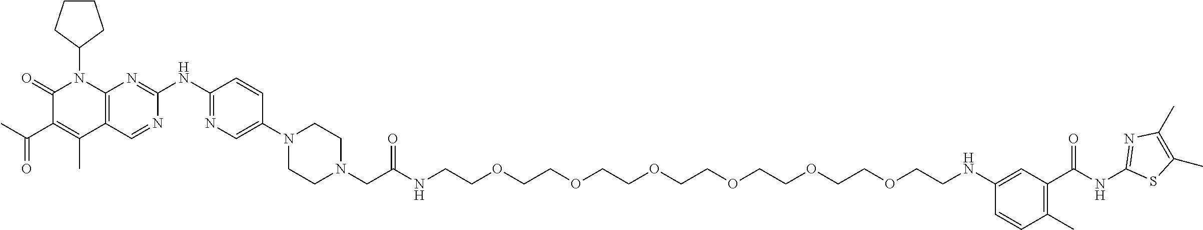 [SMILES: CC(=O)c1c(C)c2cnc(Nc3ccc(N4CCN(CC(=O)NCCOCCOCCOCCOCCOCCOCCNc5ccc(C)c(C(=O)Nc6nc(C)c(C)s6)c5)CC4)cn3)nc2n(C2CCCC2)c1=O] 5-((1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18,21-hexaoxa- 3-azatricosan-23-yl)amino)-N-(4,5-dimethylthiazol-2-yl)-2-methylbenzamide |
| CPD-133 | 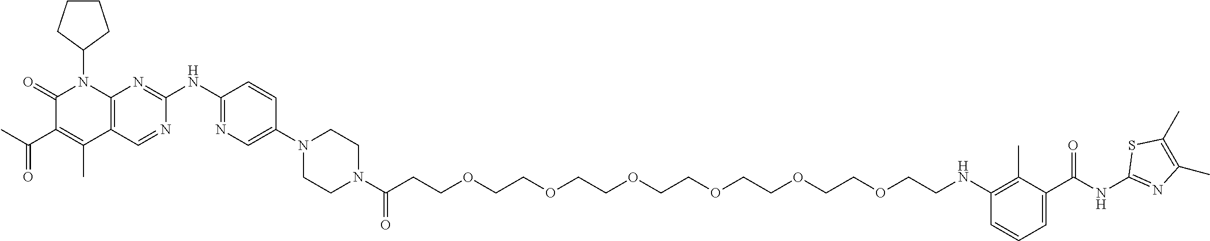 [SMILES: CC(=O)c1c(C)c2cnc(Nc3ccc(N4CCN(C(=O)CCOCCOCCOCCOCCOCCOCCNc5cccc(C(=O)Nc6nc(C)c(C)s6)c5C)CC4)cn3)nc2n(C2CCCC2)c1=O] 3-((21-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-21-oxo-3,6,9,12,15,18- hexaoxahenicosyl)amino)-N-(4,5-dimethylthiazol-2-yl)-2-methylbenzamide |
| CPD-134 | 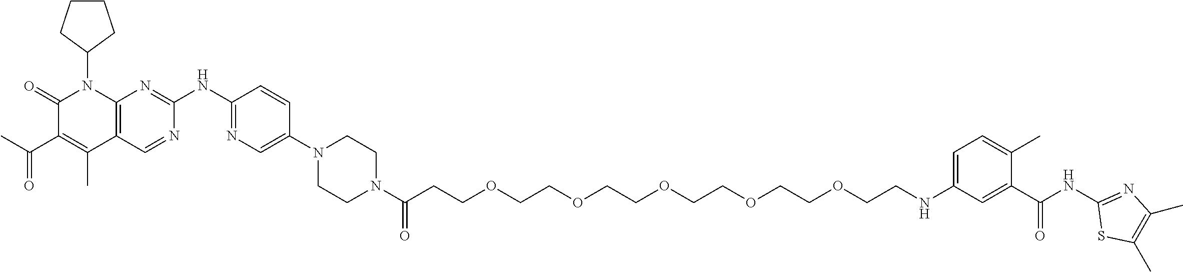 [SMILES: CC(=O)c1c(C)c2cnc(Nc3ccc(N4CCN(C(=O)CCOCCOCCOCCOCCOCCNc5ccc(C)c(C(=O)Nc6nc(C)c(C)s6)c5)CC4)cn3)nc2n(C2CCCC2)c1=O] 5-((18-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-18-oxo-3,6,9,12,15- pentaoxaoctadecyl)amino)-N-(4,5-dimethylthiazol-2-yl)-2-methylbenzamide |
| CPD-135 | 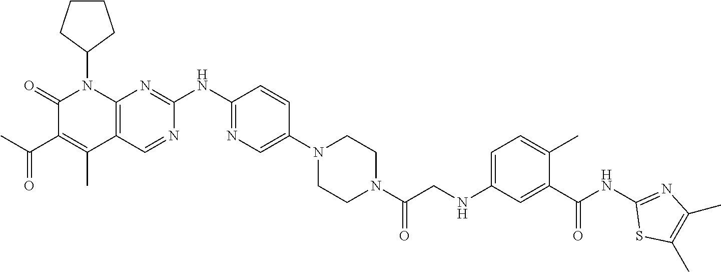 [SMILES: CC(=O)c1c(C)c2cnc(Nc3ccc(N4CCN(C(=O)CNc5ccc(C)c(C(=O)Nc6nc(C)c(C)s6)c5)CC4)cn3)nc2n(C2CCCC2)c1=O] 5-((2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxoethyl)amino)-N-(4,5- dimethylthiazol-2-yl)-2-methylbenzamide |
| CPD-136 | 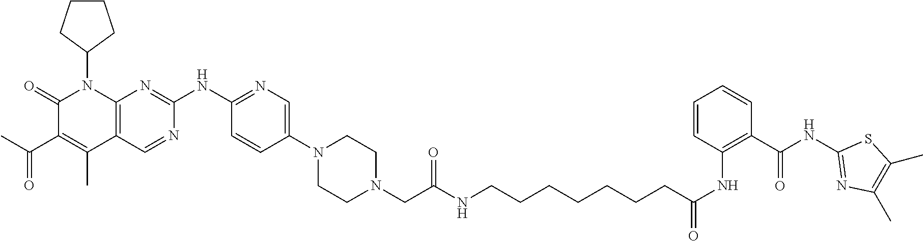 [SMILES: CC(=O)c1c(C)c2cnc(Nc3ccc(N4CCN(CC(=O)NCCCCCCCC(=O)Nc5ccccc5C(=O)Nc5nc(C)c(C)s5)CC4)cn3)nc2n(C2CCCC2)c1=O] 2-(8-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)octanamido)-N-(4,5- dimethylthiazol-2-yl)benzamide |
| CPD-137 | 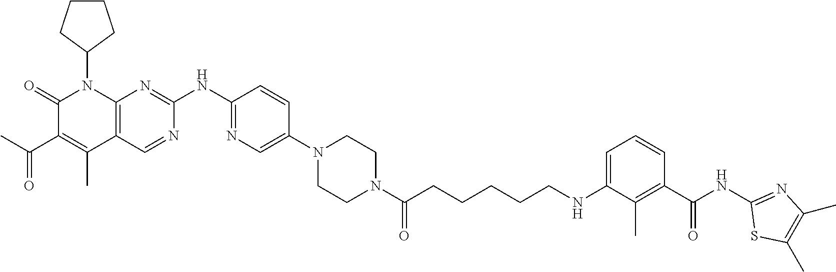 [SMILES: CC(=O)c1c(C)c2cnc(Nc3ccc(N4CCN(C(=O)CCCCCNc5cccc(C(=O)Nc6nc(C)c(C)s6)c5C)CC4)cn3)nc2n(C2CCCC2)c1=O] 3-((6-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-6-oxohexyl)amino)-N-(4,5- dimethylthiazol-2-yl)-2-methylbenzamide |
| CPD-138 | 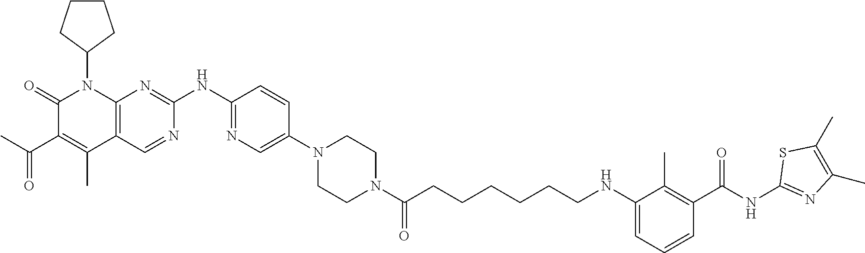 [SMILES: CC(=O)c1c(C)c2cnc(Nc3ccc(N4CCN(C(=O)CCCCCCNc5cccc(C(=O)Nc6nc(C)c(C)s6)c5C)CC4)cn3)nc2n(C2CCCC2)c1=O] 3-((7-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-7-oxoheptyl)amino)-N-(4,5- dimethylthiazol-2-yl)-2-methylbenzamide |
| CPD-139 | 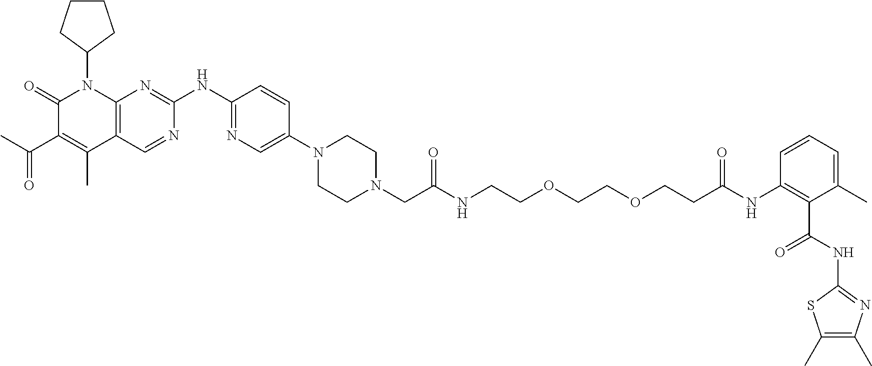 [SMILES: CC(=O)c1c(C)c2cnc(Nc3ccc(N4CCN(CC(=O)NCCOCCOCCC(=O)Nc5cccc(C)c5C(=O)Nc5nc(C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(4,5-dimethylthiazol-2-yl)-6- methylbenzamide |
| CPD-140 | 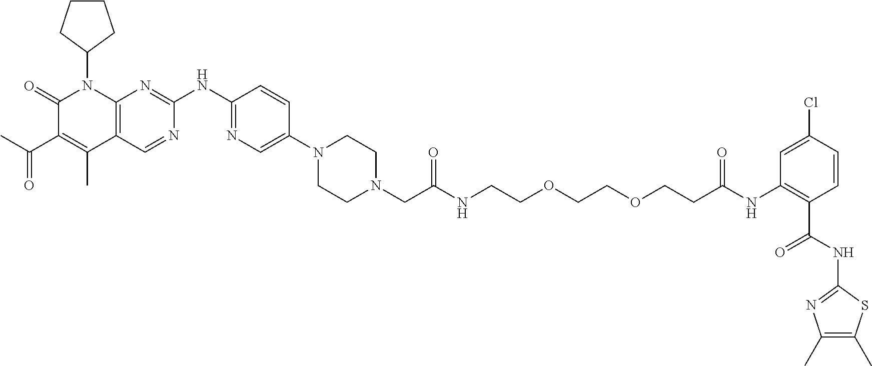 [SMILES: CC(=O)c1c(C)c2cnc(Nc3ccc(N4CCN(CC(=O)NCCOCCOCCC(=O)Nc5cc(Cl)ccc5C(=O)Nc5nc(C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-4-chloro-N-(4,5-dimethylthiazol-2- yl)benzamide |
| CPD-141 | 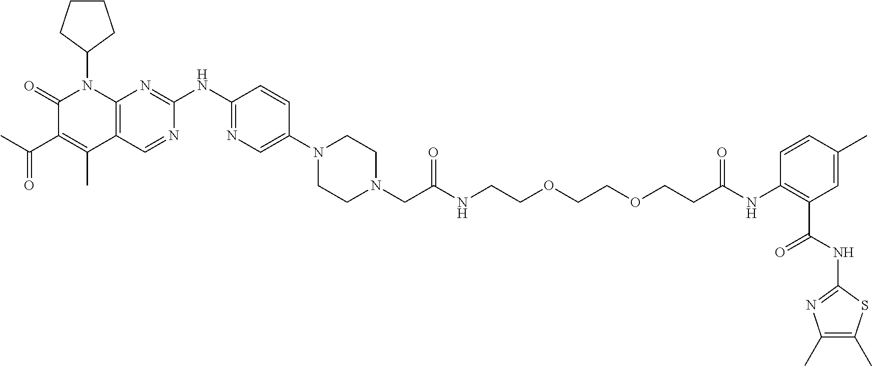 [SMILES: CC(=O)c1c(C)c2cnc(Nc3ccc(N4CCN(CC(=O)NCCOCCOCCC(=O)Nc5ccc(C)cc5C(=O)Nc5nc(C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(4,5-dimethylthiazol-2-yl)-5- methylbenzamide |
| CPD-142 | 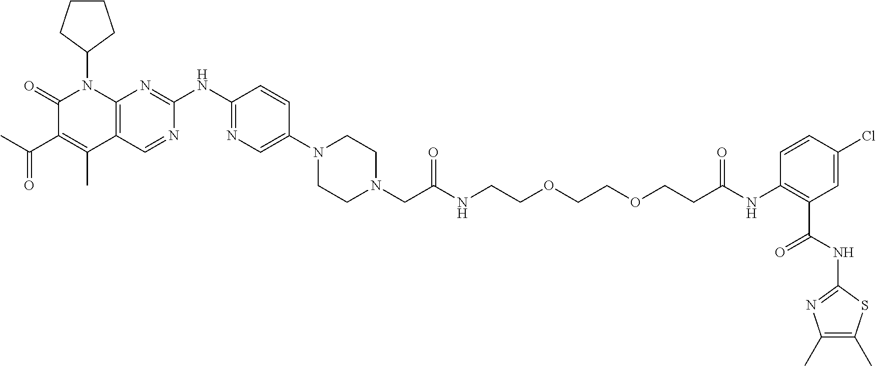 [SMILES: CC(=O)c1c(C)c2cnc(Nc3ccc(N4CCN(CC(=O)NCCOCCOCCC(=O)Nc5ccc(Cl)cc5C(=O)Nc5nc(C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-5-chloro-N-(4,5-dimethylthiazol-2- yl)benzamide |
| CPD-143 | 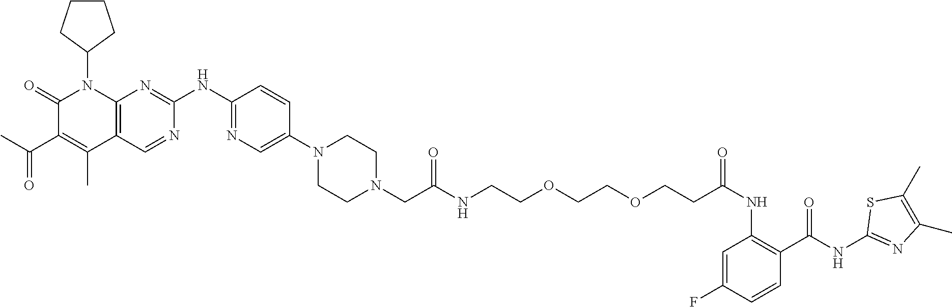 [SMILES: CC(=O)c1c(C)c2cnc(Nc3ccc(N4CCN(CC(=O)NCCOCCOCCC(=O)Nc5cc(F)ccc5C(=O)Nc5nc(C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(4,5-dimethylthiazol-2-yl)-4- fluorobenzamide |
| CPD-144 | 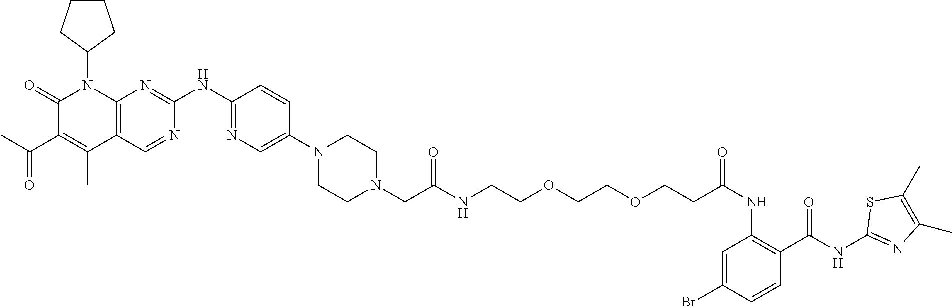 [SMILES: CC(=O)c1c(C)c2cnc(Nc3ccc(N4CCN(CC(=O)NCCOCCOCCC(=O)Nc5cc(Br)ccc5C(=O)Nc5nc(C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-4-bromo-N-(4,5-dimethylthiazol-2- yl)benzamide |
| CPD-145 | 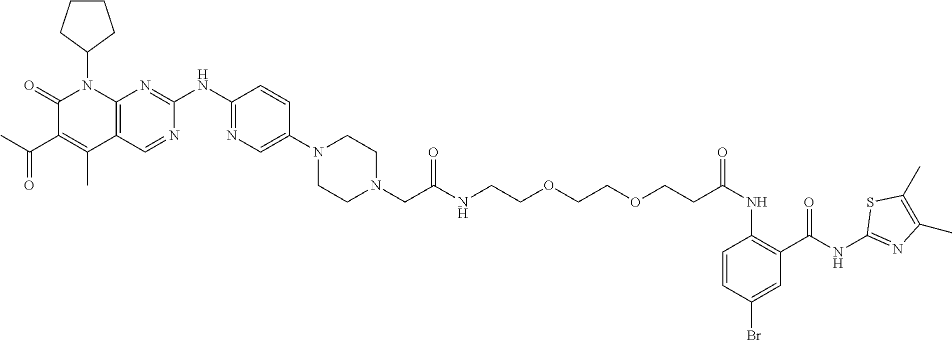 [SMILES: CC(=O)c1c(C)c2cnc(Nc3ccc(N4CCN(CC(=O)NCCOCCOCCC(=O)Nc5ccc(Br)cc5C(=O)Nc5nc(C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-5-bromo-N-(4,5-dimethylthiazol-2- yl)benzamide |
| CPD-146 | 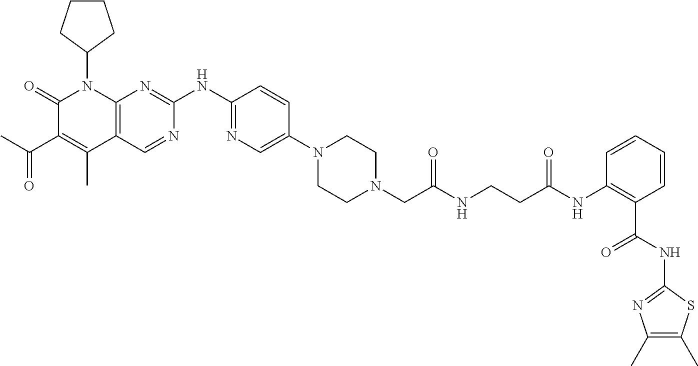 [SMILES: CC(=O)c1c(C)c2cnc(Nc3ccc(N4CCN(CC(=O)NCCC(=O)Nc5ccccc5C(=O)Nc5nc(C)c(C)s5)CC4)cn3)nc2n(C2CCCC2)c1=O] 2-(3-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)propanamido)-N-(4,5- dimethylthiazol-2-yl)benzamide |
| CPD-147 | 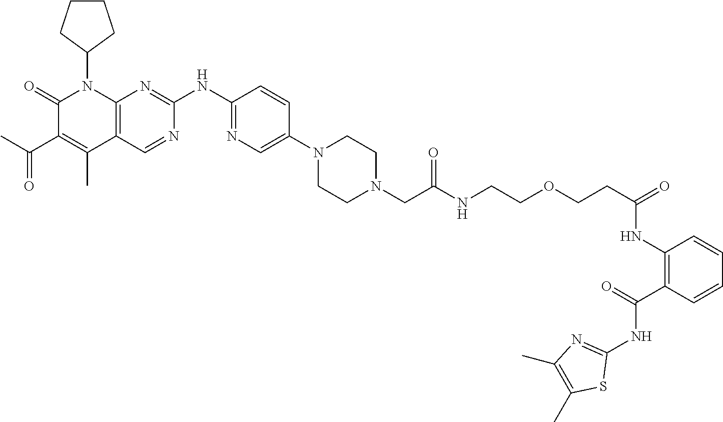 [SMILES: CC(=O)c1c(C)c2cnc(Nc3ccc(N4CCN(CC(=O)NCCOCCC(=O)Nc5ccccc5C(=O)Nc5nc(C)c(C)s5)CC4)cn3)nc2n(C2CCCC2)c1=O] 2-(3-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)ethoxy)propanamido)- N-(4,5-dimethylthiazol-2-yl)benzamide |
| CPD-148 | 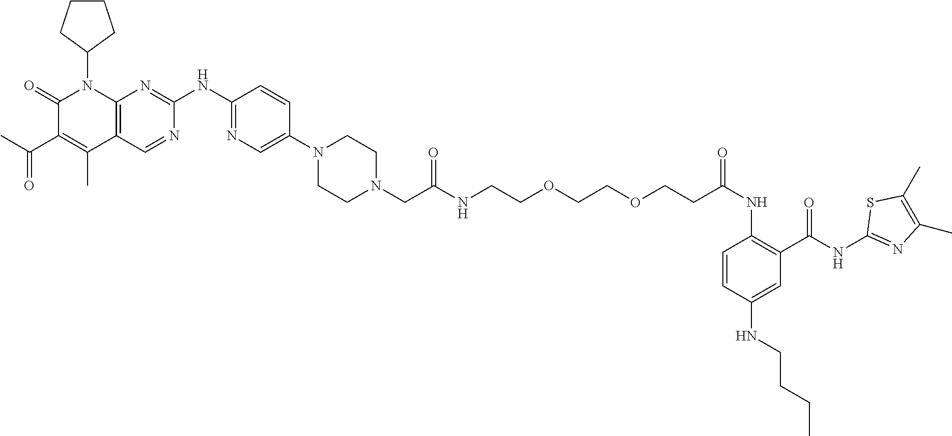 [SMILES: CCCCNc1ccc(NC(=O)CCOCCOCCNC(=O)CN2CCN(c3ccc(Nc4ncc5c(C)c(C(C)=O)c(=O)n(C6CCCC6)c5n4)nc3)CC2)c(C(=O)Nc2nc(C)c(C)s2)c1] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-5-(butylamino)-N-(4,5-dimethylthiazol-2- yl)benzamide |
| CPD-149 | 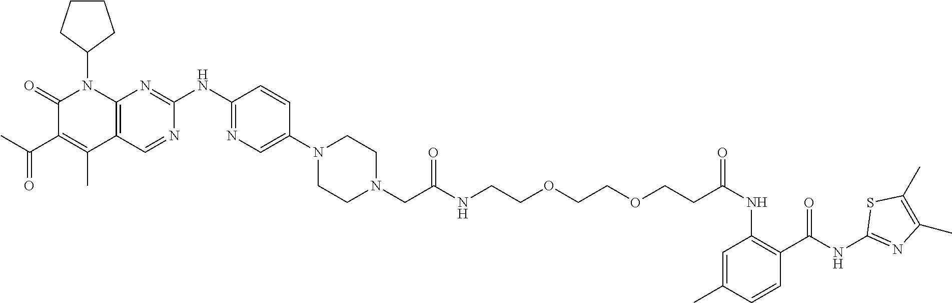 [SMILES: CC(=O)c1c(C)c2cnc(Nc3ccc(N4CCN(CC(=O)NCCOCCOCCC(=O)Nc5cc(C)ccc5C(=O)Nc5nc(C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(4,5-dimethylthiazol-2-yl)-4- methylbenzamide |
| CPD-150 | 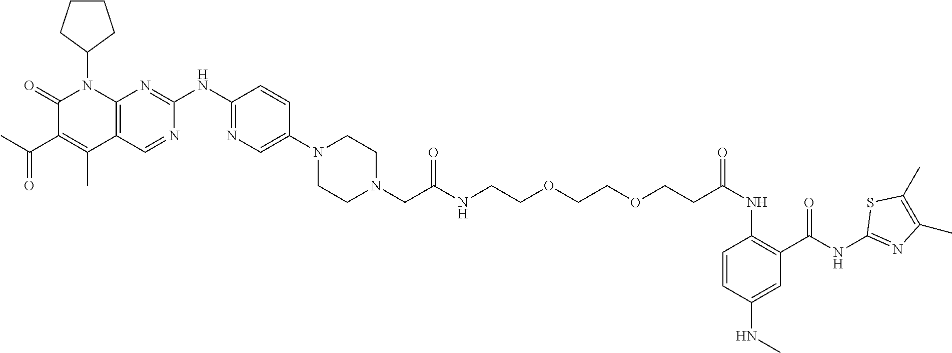 [SMILES: CNc1ccc(NC(=O)CCOCCOCCNC(=O)CN2CCN(c3ccc(Nc4ncc5c(C)c(C(C)=O)c(=O)n(C6CCCC6)c5n4)nc3)CC2)c(C(=O)Nc2nc(C)c(C)s2)c1] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(4,5-dimethylthiazol-2-yl)-5- (methylamino)benzamide |
| CPD-151 | 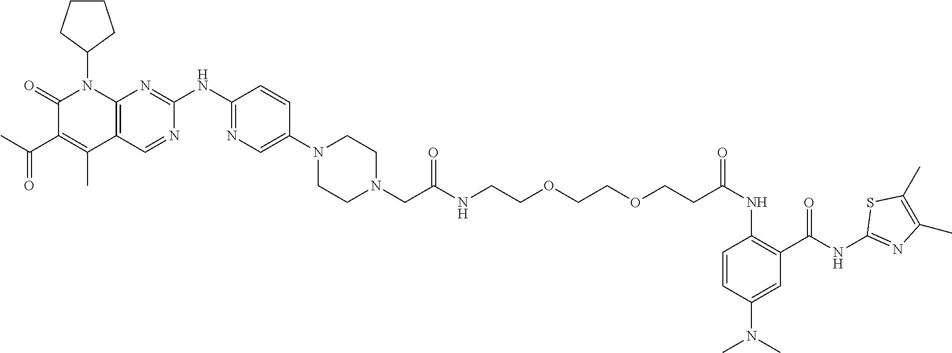 [SMILES: CC(=O)c1c(C)c2cnc(Nc3ccc(N4CCN(CC(=O)NCCOCCOCCC(=O)Nc5ccc(N(C)C)cc5C(=O)Nc5nc(C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-5-(dimethylamino)-N-(4,5- dimethylthiazol-2-yl)benzamide |
| CPD-152 | 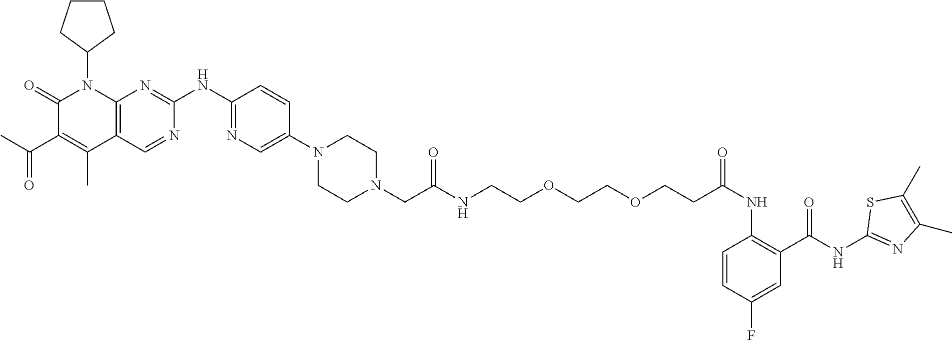 [SMILES: CC(=O)c1c(C)c2cnc(Nc3ccc(N4CCN(CC(=O)NCCOCCOCCC(=O)Nc5ccc(F)cc5C(=O)Nc5nc(C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(4,5-dimethylthiazol-2-yl)-5- fluorobenzamide |
| CPD-153 | 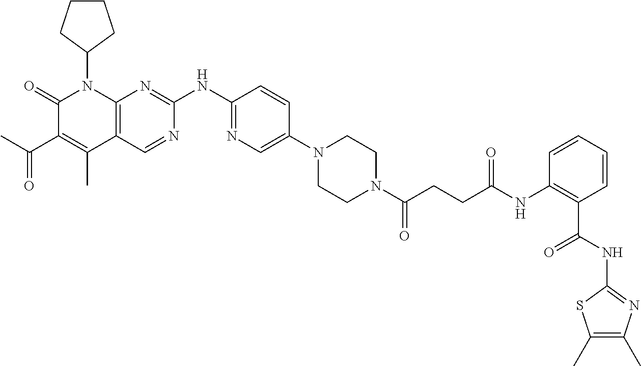 [SMILES: CC(=O)c1c(C)c2cnc(Nc3ccc(N4CCN(C(=O)CCC(=O)Nc5ccccc5C(=O)Nc5nc(C)c(C)s5)CC4)cn3)nc2n(C2CCCC2)c1=O] 2-(4-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-4-oxobutanamido)-N-(4,5- dimethylthiazol-2-yl)benzamide |
| CPD-154 | 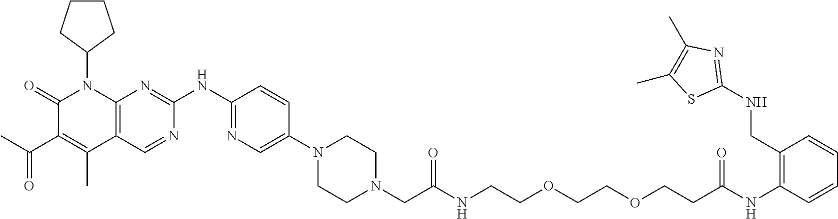 [SMILES: CC(=O)c1c(C)c2cnc(Nc3ccc(N4CCN(CC(=O)NCCOCCOCCC(=O)Nc5ccccc5CNc5nc(C)c(C)s5)CC4)cn3)nc2n(C2CCCC2)c1=O] 3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)ethoxy)ethoxy)-N-(2- (((4,5-dimethylthiazol-2-yl)amino)methyl)phenyl)propanamide |
| CPD-155 | 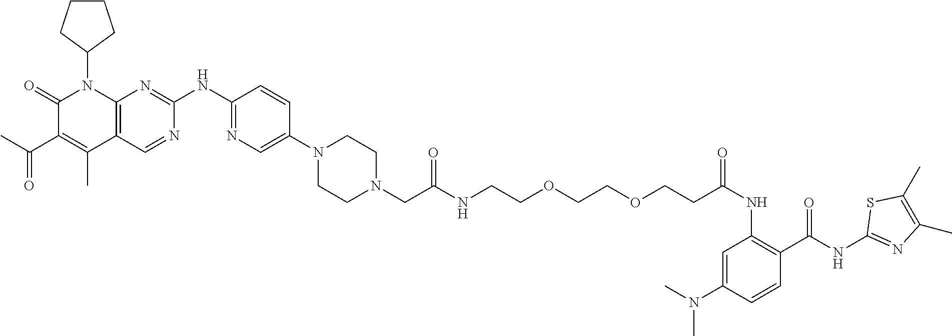 [SMILES: CC(=O)c1c(C)c2cnc(Nc3ccc(N4CCN(CC(=O)NCCOCCOCCC(=O)Nc5cc(N(C)C)ccc5C(=O)Nc5nc(C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-4-(dimethylamino)-N-(4,5- dimethylthiazol-2-yl)benzamide |
| CPD-156 | 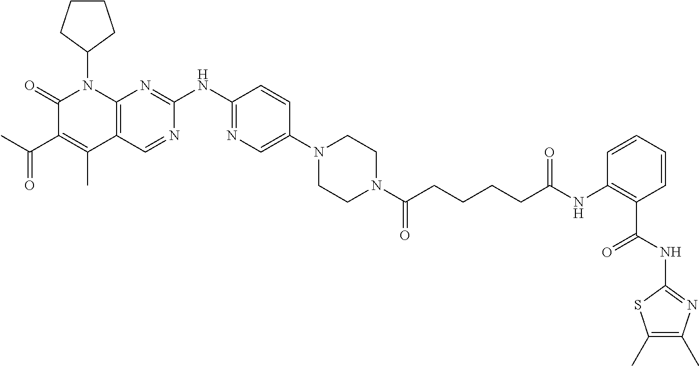 [SMILES: CC(=O)c1c(C)c2cnc(Nc3ccc(N4CCN(C(=O)CCCCC(=O)Nc5ccccc5C(=O)Nc5nc(C)c(C)s5)CC4)cn3)nc2n(C2CCCC2)c1=O] 2-(6-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-6-oxohexanamido)-N-(4,5- dimethylthiazol-2-yl)benzamide |
| CPD-157 | 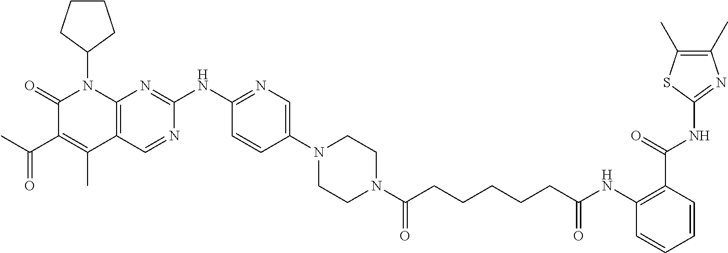 [SMILES: CC(=O)c1c(C)c2cnc(Nc3ccc(N4CCN(C(=O)CCCCCC(=O)Nc5ccccc5C(=O)Nc5nc(C)c(C)s5)CC4)cn3)nc2n(C2CCCC2)c1=O] 2-(7-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-7-oxoheptanamido)-N-(4,5- dimethylthiazol-2-yl)benzamide |
| CPD-158 | 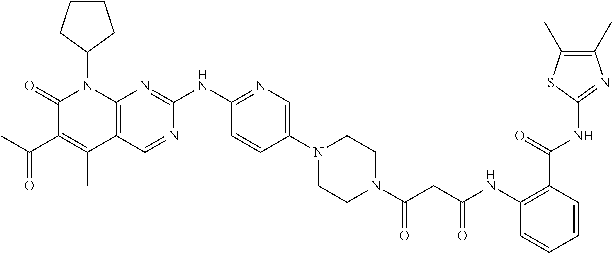 [SMILES: CC(=O)c1c(C)c2cnc(Nc3ccc(N4CCN(C(=O)CC(=O)Nc5ccccc5C(=O)Nc5nc(C)c(C)s5)CC4)cn3)nc2n(C2CCCC2)c1=O] 2-(3-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-3-oxopropanamido)-N-(4,5- dimethylthiazol-2-yl)benzamide |
| CPD-159 | 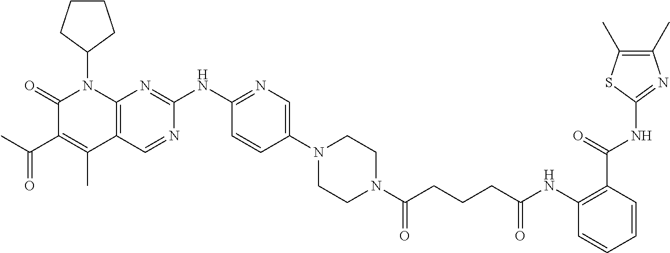 [SMILES: CC(=O)c1c(C)c2cnc(Nc3ccc(N4CCN(C(=O)CCCC(=O)Nc5ccccc5C(=O)Nc5nc(C)c(C)s5)CC4)cn3)nc2n(C2CCCC2)c1=O] 2-(5-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-5-oxopentanamido)-N-(4,5- dimethylthiazol-2-yl)benzamide |
| CPD-160 | 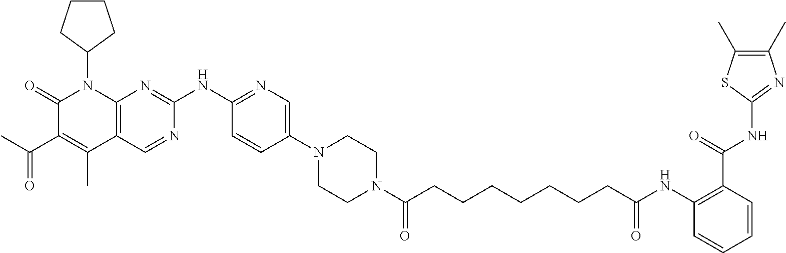 [SMILES: CC(=O)c1c(C)c2cnc(Nc3ccc(N4CCN(C(=O)CCCCCCCC(=O)Nc5ccccc5C(=O)Nc5nc(C)c(C)s5)CC4)cn3)nc2n(C2CCCC2)c1=O] 2-(9-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-9-oxononanamido)-N-(4,5- dimethylthiazol-2-yl)benzamide |
| CPD-161 | 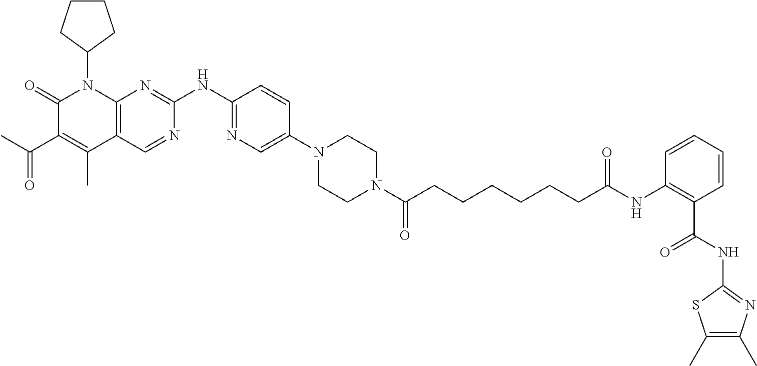 [SMILES: CC(=O)c1c(C)c2cnc(Nc3ccc(N4CCN(C(=O)CCCCCCC(=O)Nc5ccccc5C(=O)Nc5nc(C)c(C)s5)CC4)cn3)nc2n(C2CCCC2)c1=O] 2-(8-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-8-oxooctanamido)-N-(4,5- dimethylthiazol-2-yl)benzamide |
| CPD-162 | 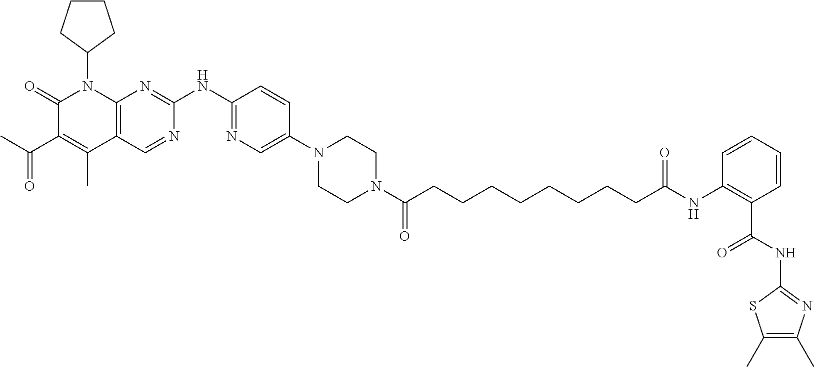 [SMILES: CC(=O)c1c(C)c2cnc(Nc3ccc(N4CCN(C(=O)CCCCCCCCC(=O)Nc5ccccc5C(=O)Nc5nc(C)c(C)s5)CC4)cn3)nc2n(C2CCCC2)c1=O] 2-(10-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-10-oxodecanamido)-N-(4,5- dimethylthiazol-2-yl)benzamide |
| CPD-163 | 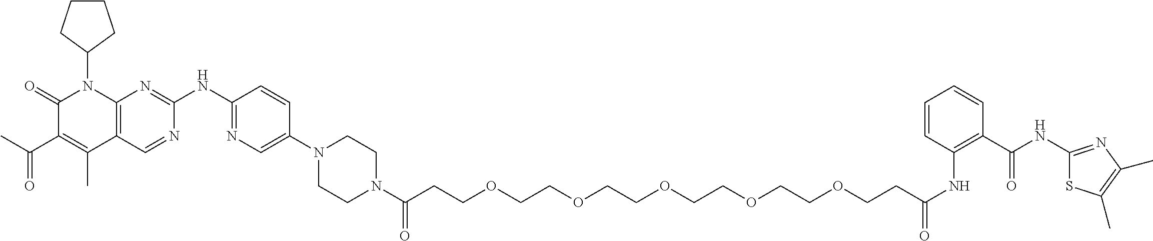 [SMILES: CC(=O)c1c(C)c2cnc(Nc3ccc(N4CCN(C(=O)CCOCCOCCOCCOCCOCCC(=O)Nc5ccccc5C(=O)Nc5nc(C)c(C)s5)CC4)cn3)nc2n(C2CCCC2)c1=O] 19-(4-(6-((6-acetyl-8-cyclopentyl-5-methyl-7-oxo-7,8-dihydropyrido[2,3-d]pyrimidin- 2-yl)amino)pyridin-3-yl)piperazin-1-yl)-N-(2-((4,5-dimethylthiazol-2- yl)carbamoyl)phenyl)-19-oxo-4,7,10,13,16-pentaoxanonadecanamide |
| CPD-164 | 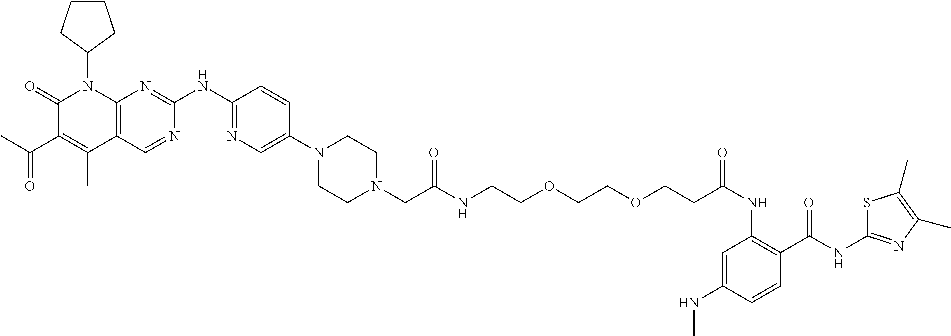 [SMILES: CNc1ccc(C(=O)Nc2nc(C)c(C)s2)c(NC(=O)CCOCCOCCNC(=O)CN2CCN(c3ccc(Nc4ncc5c(C)c(C(C)=O)c(=O)n(C6CCCC6)c5n4)nc3)CC2)c1] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(4,5-dimethylthiazol-2-yl)-4- (methylamino)benzamide |
| CPD-165 | 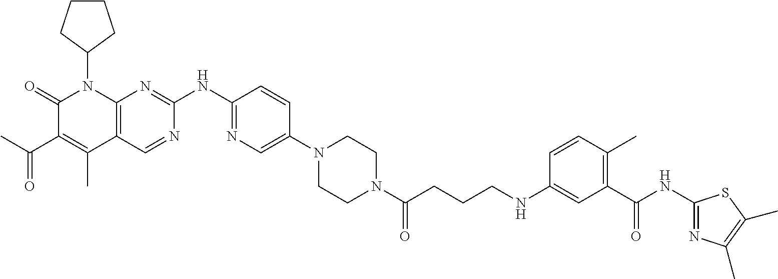 [SMILES: CC(=O)c1c(C)c2cnc(Nc3ccc(N4CCN(C(=O)CCCNc5ccc(C)c(C(=O)Nc6nc(C)c(C)s6)c5)CC4)cn3)nc2n(C2CCCC2)c1=O] 5-((4-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-4-oxobutyl)amino)-N-(4,5- dimethylthiazol-2-yl)-2-methylbenzamide |
| CPD-166 | 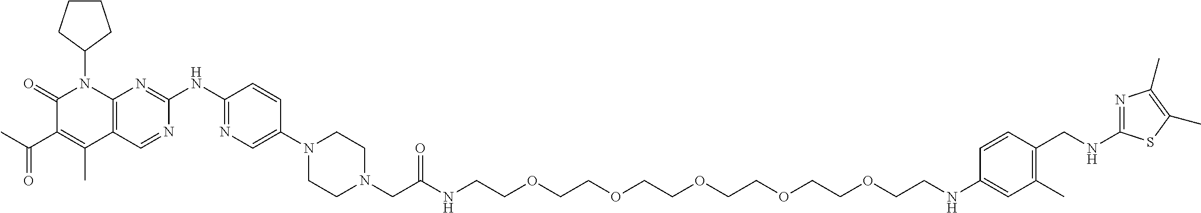 [SMILES: CC(=O)c1c(C)c2cnc(Nc3ccc(N4CCN(CC(=O)NCCOCCOCCOCCOCCOCCNc5ccc(CNc6nc(C)c(C)s6)c(C)c5)CC4)cn3)nc2n(C2CCCC2)c1=O] 2-(4-(6-((6-acetyl-8-cyclopentyl-5-methyl-7-oxo-7,8-dihydropyrido[2,3-d]pyrimidin- 2-yl)amino)pyridin-3-yl)piperazin-1-yl)-N-(17-((4-(((4,5-dimethylthiazol-2- yl)amino)methyl)-3-methylphenyl)amino)-3,6,9,12,15-pentaoxaheptadecyl)acetamide |
| CPD-167 | 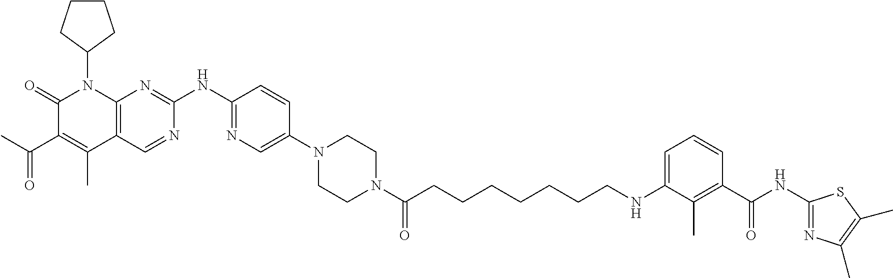 [SMILES: CC(=O)c1c(C)c2cnc(Nc3ccc(N4CCN(C(=O)CCCCCCCNc5cccc(C(=O)Nc6nc(C)c(C)s6)c5C)CC4)cn3)nc2n(C2CCCC2)c1=O] 3-((8-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-8-oxooctyl)amino)-N-(4,5- dimethylthiazol-2-yl)-2-methylbenzamide |
| CPD-168 | 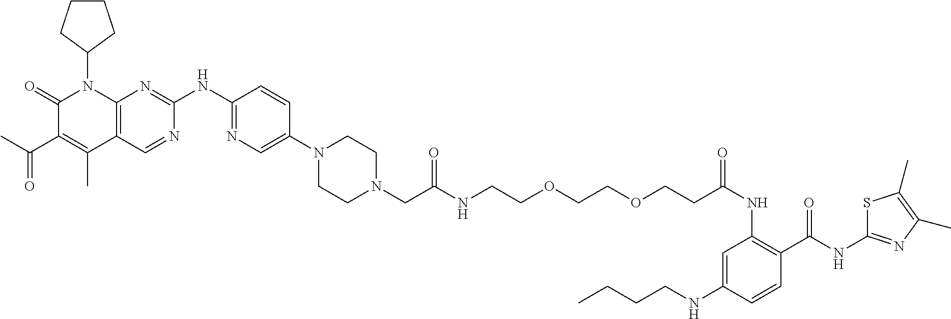 [SMILES: CCCCNc1ccc(C(=O)Nc2nc(C)c(C)s2)c(NC(=O)CCOCCOCCNC(=O)CN2CCN(c3ccc(Nc4ncc5c(C)c(C(C)=O)c(=O)n(C6CCCC6)c5n4)nc3)CC2)c1] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-4-(butylamino)-N-(4,5-dimethylthiazol-2- yl)benzamide |
| CPD-169 | 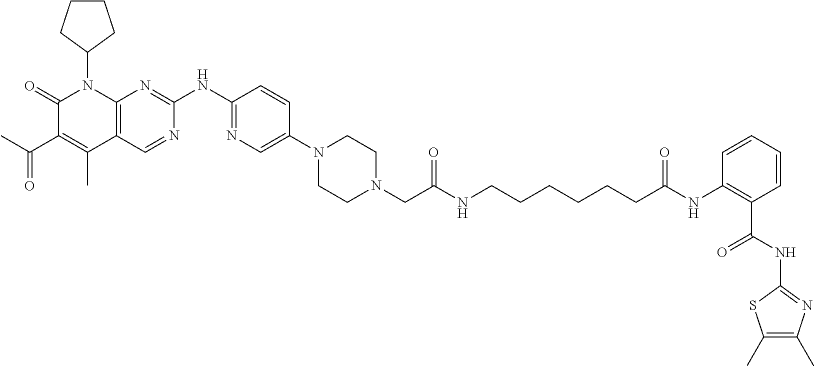 [SMILES: CC(=O)c1c(C)c2cnc(Nc3ccc(N4CCN(CC(=O)NCCCCCCC(=O)Nc5ccccc5C(=O)Nc5nc(C)c(C)s5)CC4)cn3)nc2n(C2CCCC2)c1=O] 2-(7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptanamido)-N-(4,5- dimethylthiazol-2-yl)benzamide |
| CPD-170 | 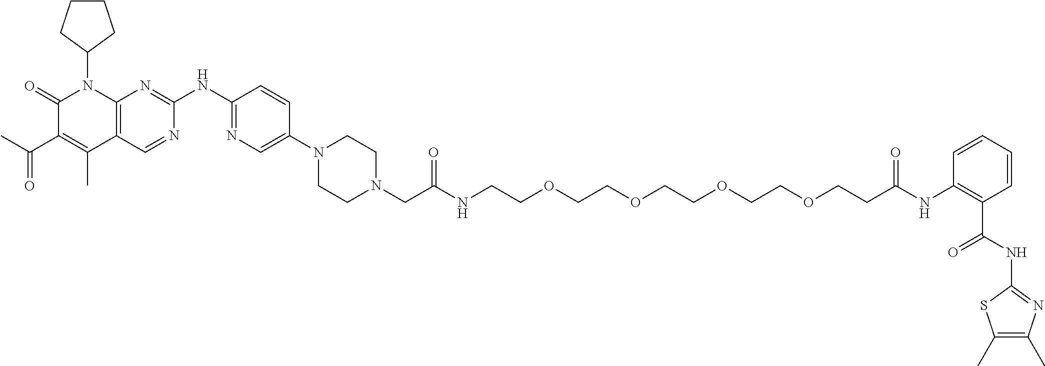 [SMILES: CC(=O)c1c(C)c2cnc(Nc3ccc(N4CCN(CC(=O)NCCOCCOCCOCCOCCC(=O)Nc5ccccc5C(=O)Nc5nc(C)c(C)s5)CC4)cn3)nc2n(C2CCCC2)c1=O] 2-(1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15-tetraoxa-3- azaoctadecan-18-amido)-N-(4,5-dimethylthiazol-2-yl)benzamide |
| CPD-171 | 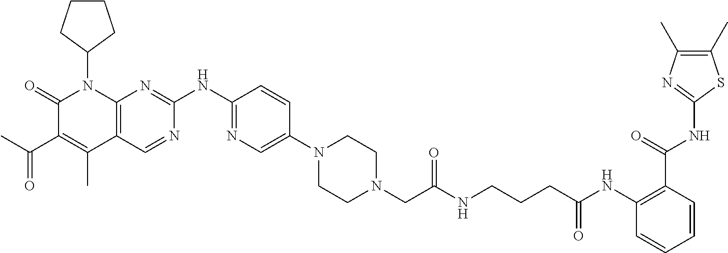 [SMILES: CC(=O)c1c(C)c2cnc(Nc3ccc(N4CCN(CC(=O)NCCCC(=O)Nc5ccccc5C(=O)Nc5nc(C)c(C)s5)CC4)cn3)nc2n(C2CCCC2)c1=O] 2-(4-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)butanamido)-N-(4,5- dimethylthiazol-2-yl)benzamide |
| CPD-172 | 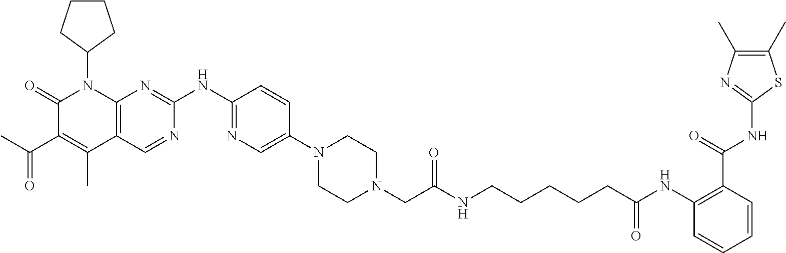 [SMILES: CC(=O)c1c(C)c2cnc(Nc3ccc(N4CCN(CC(=O)NCCCCCC(=O)Nc5ccccc5C(=O)Nc5nc(C)c(C)s5)CC4)cn3)nc2n(C2CCCC2)c1=O] 2-(6-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)hexanamido)-N-(4,5- dimethylthiazol-2-yl)benzamide |
| CPD-173 | 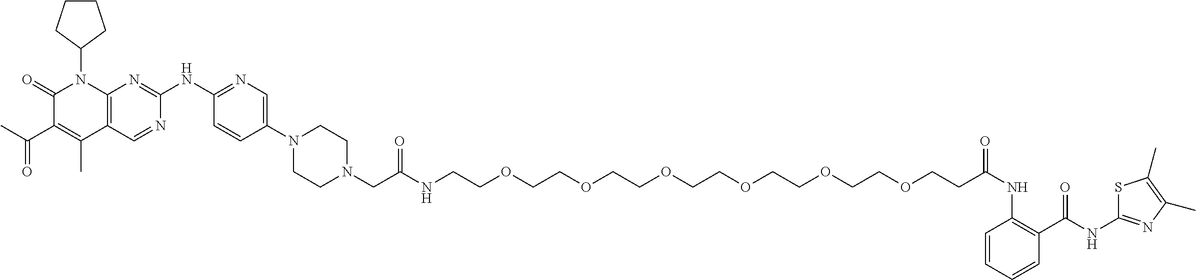 [SMILES: CC(=O)c1c(C)c2cnc(Nc3ccc(N4CCN(CC(=O)NCCOCCOCCOCCOCCOCCOCCC(=O)Nc5ccccc5C(=O)Nc5nc(C)c(C)s5)CC4)cn3)nc2n(C2CCCC2)c1=O] 2-(1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18,21-hexaoxa- 3-azatetracosan-24-amido)-N-(4,5-dimethylthiazol-2-yl)benzamide |
| CPD-174 | 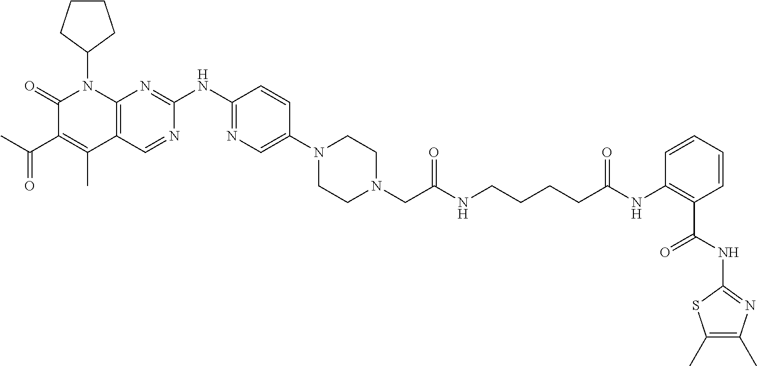 [SMILES: CC(=O)c1c(C)c2cnc(Nc3ccc(N4CCN(CC(=O)NCCCCC(=O)Nc5ccccc5C(=O)Nc5nc(C)c(C)s5)CC4)cn3)nc2n(C2CCCC2)c1=O] 2-(5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentanamido)-N-(4,5- dimethylthiazol-2-yl)benzamide |
| CPD-175 | 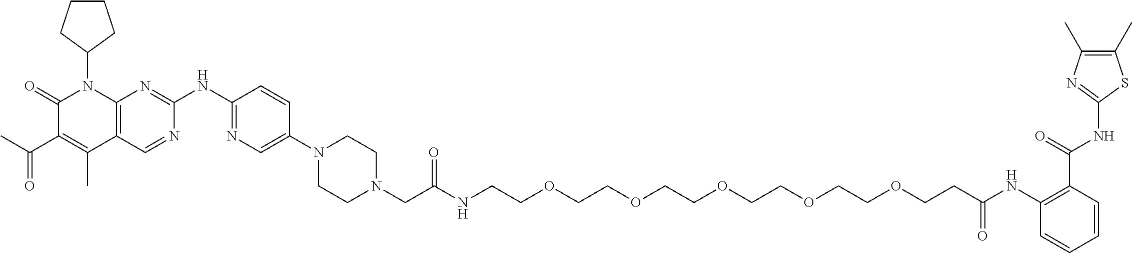 [SMILES: CC(=O)c1c(C)c2cnc(Nc3ccc(N4CCN(CC(=O)NCCOCCOCCOCCOCCOCCC(=O)Nc5ccccc5C(=O)Nc5nc(C)c(C)s5)CC4)cn3)nc2n(C2CCCC2)c1=O] 2-(1-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-2-oxo-6,9,12,15,18-pentaoxa-3- azahenicosan-21-amido)-N-(4,5-dimethylthiazol-2-yl)benzamide |
| CPD-176 | 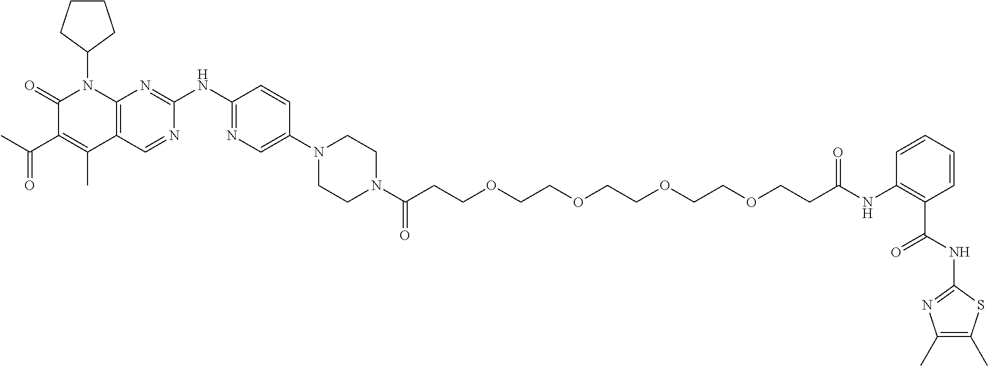 [SMILES: CC(=O)c1c(C)c2cnc(Nc3ccc(N4CCN(C(=O)CCOCCOCCOCCOCCC(=O)Nc5ccccc5C(=O)Nc5nc(C)c(C)s5)CC4)cn3)nc2n(C2CCCC2)c1=O] 16-(4-(6-((6-acetyl-8-cyclopentyl-5-methyl-7-oxo-7,8-dihydropyrido[2,3-d]pyrimidin- 2-yl)amino)pyridin-3-yl)piperazin-1-yl)-N-(2-((4,5-dimethylthiazol-2- yl)carbamoyl)phenyl)-16-oxo-4,7,10,13-tetraoxahexadecanamide |
| CPD-177 | 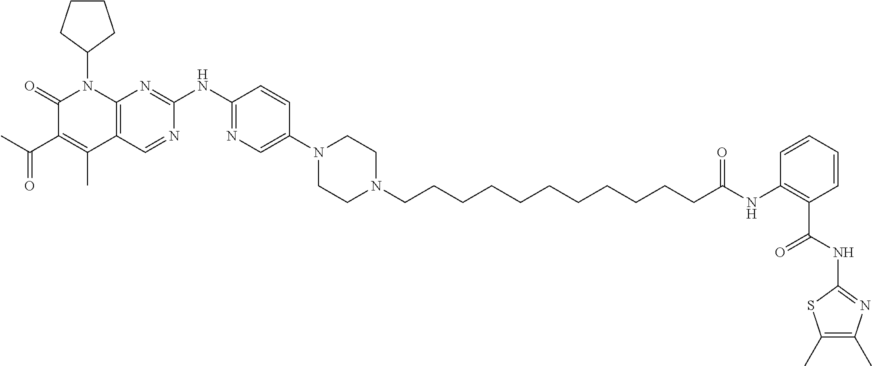 [SMILES: CC(=O)c1c(C)c2cnc(Nc3ccc(N4CCN(CCCCCCCCCCCC(=O)Nc5ccccc5C(=O)Nc5nc(C)c(C)s5)CC4)cn3)nc2n(C2CCCC2)c1=O] 2-(12-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)dodecanamido)-N-(4,5- dimethylthiazol-2-yl)benzamide |
| CPD-178 | 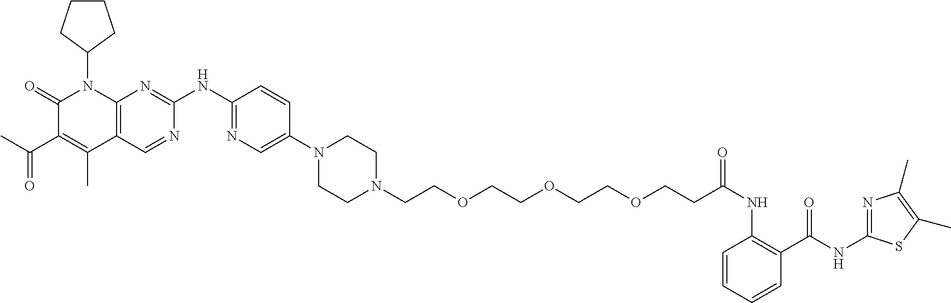 [SMILES: CC(=O)c1c(C)c2cnc(Nc3ccc(N4CCN(CCOCCOCCOCCC(=O)Nc5ccccc5C(=O)Nc5nc(C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)ethoxy)ethoxy)ethoxy)propanamido)-N-(4,5-dimethylthiazol-2-yl)benzamide |
| CPD-179 | 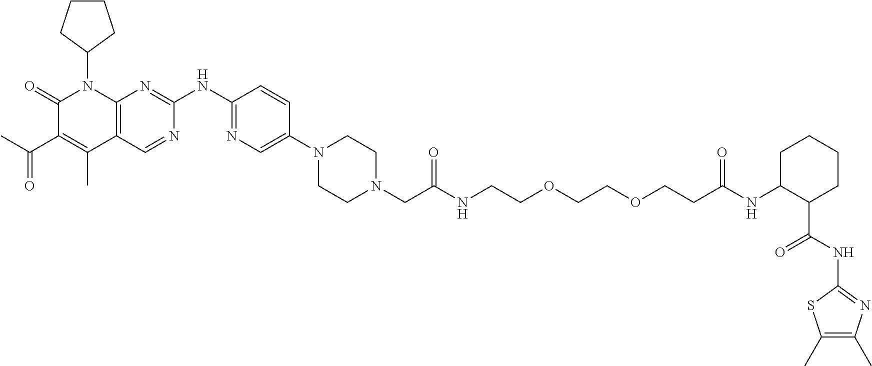 [SMILES: CC(=O)c1c(C)c2cnc(Nc3ccc(N4CCN(CC(=O)NCCOCCOCCC(=O)NC5CCCCC5C(=O)Nc5nc(C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(4,5-dimethylthiazol-2-yl)cyclohexane- 1-carboxamide |
| CPD-180 | 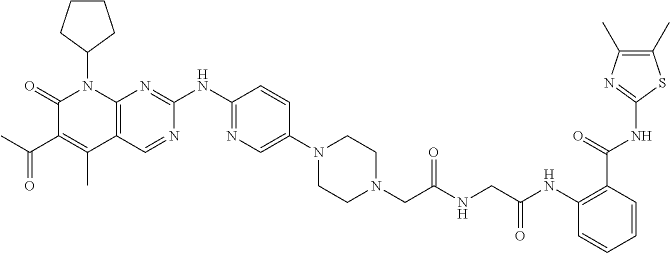 [SMILES: CC(=O)c1c(C)c2cnc(Nc3ccc(N4CCN(CC(=O)NCC(=O)Nc5ccccc5C(=O)Nc5nc(C)c(C)s5)CC4)cn3)nc2n(C2CCCC2)c1=O] 2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)acetamido)-N-(4,5- dimethylthiazol-2-yl)benzamide |
| CPD-181 | 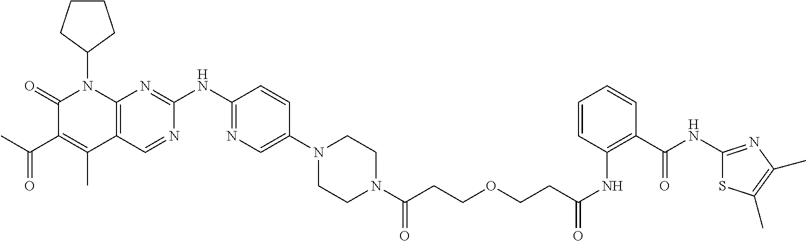 [SMILES: CC(=O)c1c(C)c2cnc(Nc3ccc(N4CCN(C(=O)CCOCCC(=O)Nc5ccccc5C(=O)Nc5nc(C)c(C)s5)CC4)cn3)nc2n(C2CCCC2)c1=O] 2-(3-(3-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-3-oxopropoxy)propanamido)-N- (4,5-dimethylthiazol-2-yl)benzamide |
| CPD-182 | 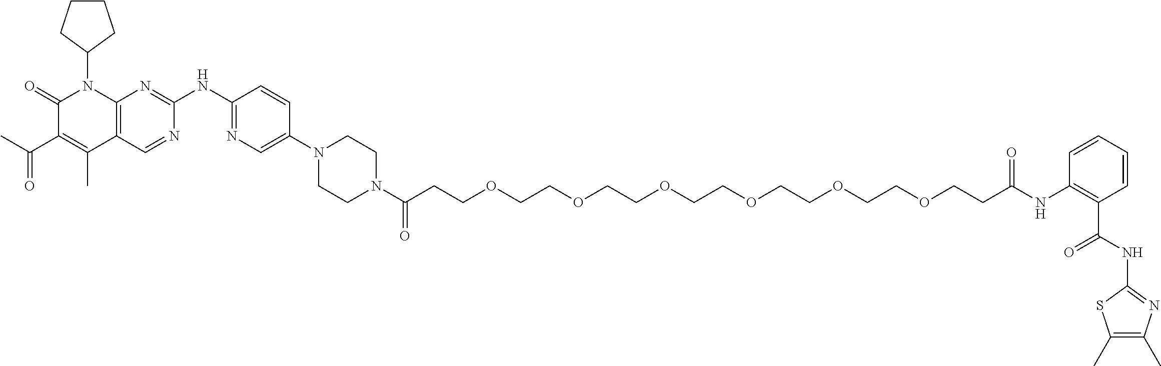 [SMILES: CC(=O)c1c(C)c2cnc(Nc3ccc(N4CCN(C(=O)CCOCCOCCOCCOCCOCCOCCC(=O)Nc5ccccc5C(=O)Nc5nc(C)c(C)s5)CC4)cn3)nc2n(C2CCCC2)c1=O] 22-(4-(6-((6-acetyl-8-cyclopentyl-5-methyl-7-oxo-7,8-dihydropyrido[2,3-d]pyrimidin- 2-yl)amino)pyridin-3-yl)piperazin-1-yl)-N-(2-((4,5-dimethylthiazol-2- yl)carbamoyl)phenyl)-22-oxo-4,7,10,13,16,19-hexaoxadocosanamide |
| CPD-183 | 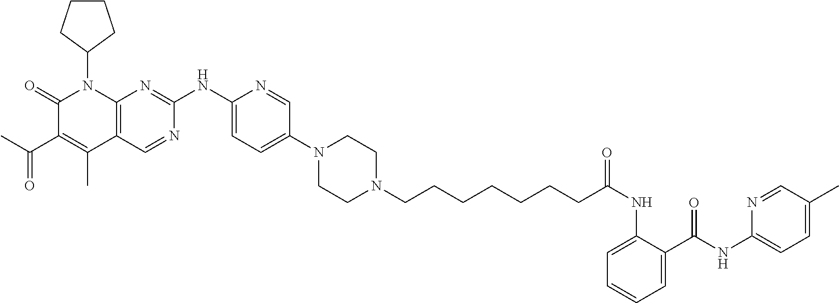 [SMILES: CC(=O)c1c(C)c2cnc(Nc3ccc(N4CCN(CCCCCCCC(=O)Nc5ccccc5C(=O)Nc5ccc(C)cn5)CC4)cn3)nc2n(C2CCCC2)c1=O] 2-(8-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)octanamido)-N-(5-methylpyridin- 2-yl)benzamide |
| CPD-184 | 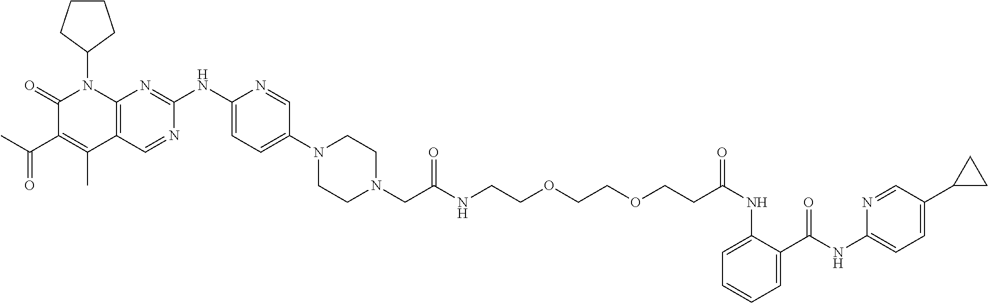 [SMILES: CC(=O)c1c(C)c2cnc(Nc3ccc(N4CCN(CC(=O)NCCOCCOCCC(=O)Nc5ccccc5C(=O)Nc5ccc(C6CC6)cn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-cyclopropylpyridin-2-yl)benzamide |
| CPD-185 | 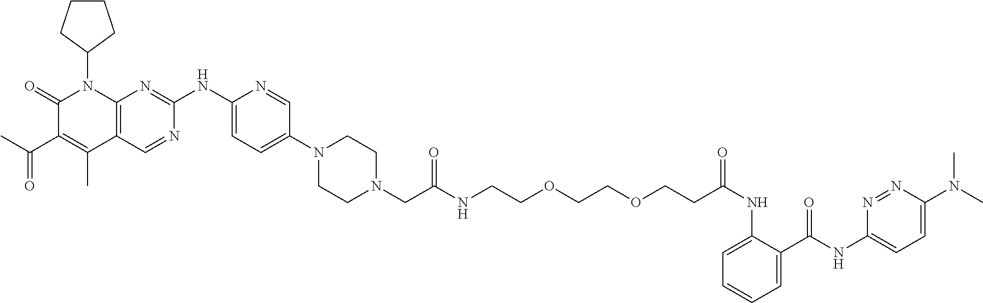 [SMILES: CC(=O)c1c(C)c2cnc(Nc3ccc(N4CCN(CC(=O)NCCOCCOCCC(=O)Nc5ccccc5C(=O)Nc5ccc(N(C)C)nn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(6-(dimethylamino)pyridazin-3- yl)benzamide |
| CPD-186 | 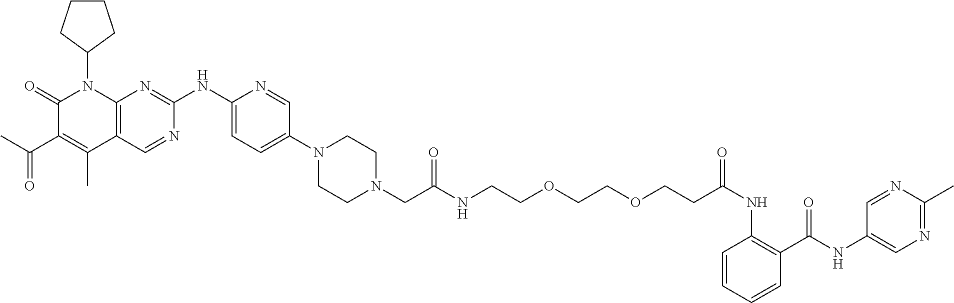 [SMILES: CC(=O)c1c(C)c2cnc(Nc3ccc(N4CCN(CC(=O)NCCOCCOCCC(=O)Nc5ccccc5C(=O)Nc5cnc(C)nc5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(2-methylpyrimidin-5-yl)benzamide |
| CPD-187 | 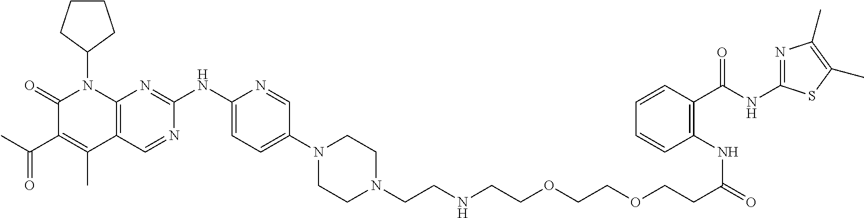 [SMILES: CC(=O)c1c(C)c2cnc(Nc3ccc(N4CCN(CCNCCOCCOCCC(=O)Nc5ccccc5C(=O)Nc5nc(C)c(C)s5)CC4)cn3)nc2n(C2CCCC2)c1=O] 2-(3-(2-(2-((2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)ethyl)amino)ethoxy)ethoxy)propanamido)-N-(4,5-dimethylthiazol-2-yl)benzamide |
| CPD-188 | 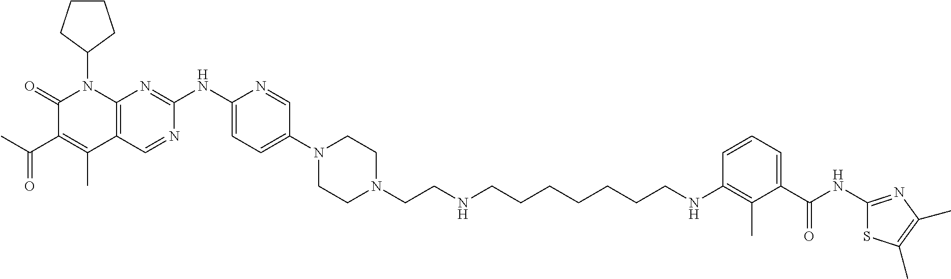 [SMILES: CC(=O)c1c(C)c2cnc(Nc3ccc(N4CCN(CCNCCCCCCCNc5cccc(C(=O)Nc6nc(C)c(C)s6)c5C)CC4)cn3)nc2n(C2CCCC2)c1=O] 3-((7-((2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)ethyl)amino)heptyl)amino)-N- (4,5-dimethylthiazol-2-yl)-2-methylbenzamide |
| CPD-189 | 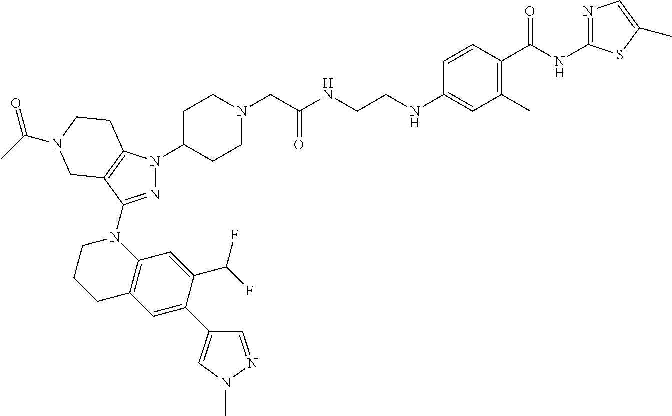 [SMILES: CC(=O)N1CCc2c(c(N3CCCc4cc(-c5cnn(C)c5)c(C(F)F)cc43)nn2C2CCN(CC(=O)NCCNc3ccc(C(=O)Nc4ncc(C)s4)c(C)c3)CC2)C1] 4-((2-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)ethyl)amino)-2-methyl-N-(5-methylthiazol-2- yl)benzamide |
| CPD-190 | 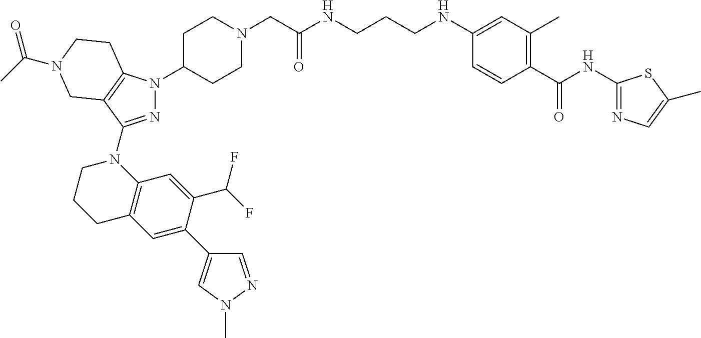 [SMILES: CC(=O)N1CCc2c(c(N3CCCc4cc(-c5cnn(C)c5)c(C(F)F)cc43)nn2C2CCN(CC(=O)NCCCNc3ccc(C(=O)Nc4ncc(C)s4)c(C)c3)CC2)C1] 4-((3-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)propyl)amino)-2-methyl-N-(5-methylthiazol-2- yl)benzamide |
| CPD-191 | 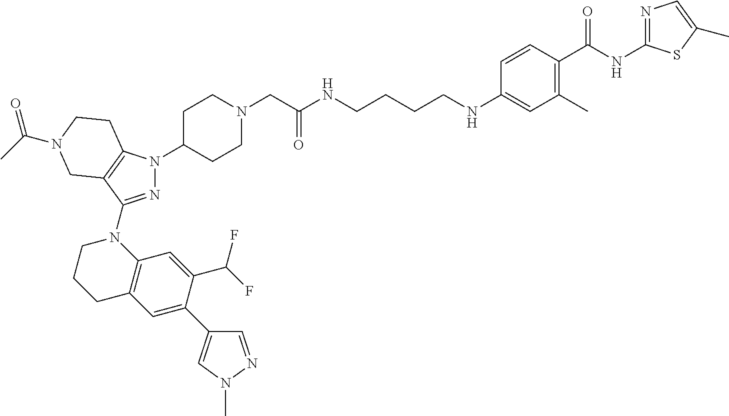 [SMILES: CC(=O)N1CCc2c(c(N3CCCc4cc(-c5cnn(C)c5)c(C(F)F)cc43)nn2C2CCN(CC(=O)NCCCCNc3ccc(C(=O)Nc4ncc(C)s4)c(C)c3)CC2)C1] 4-((4-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)butyl)amino)-2-methyl-N-(5-methylthiazol-2- yl)benzamide |
| CPD-192 | 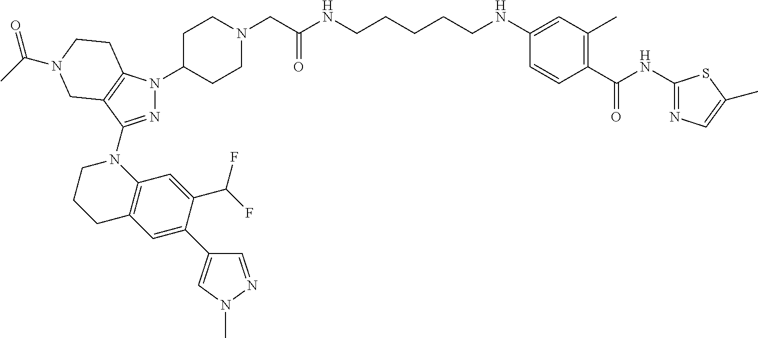 [SMILES: CC(=O)N1CCc2c(c(N3CCCc4cc(-c5cnn(C)c5)c(C(F)F)cc43)nn2C2CCN(CC(=O)NCCCCCNc3ccc(C(=O)Nc4ncc(C)s4)c(C)c3)CC2)C1] 4-((5-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)pentyl)amino)-2-methyl-N-(5-methylthiazol-2- yl)benzamide |
| CPD-193 | 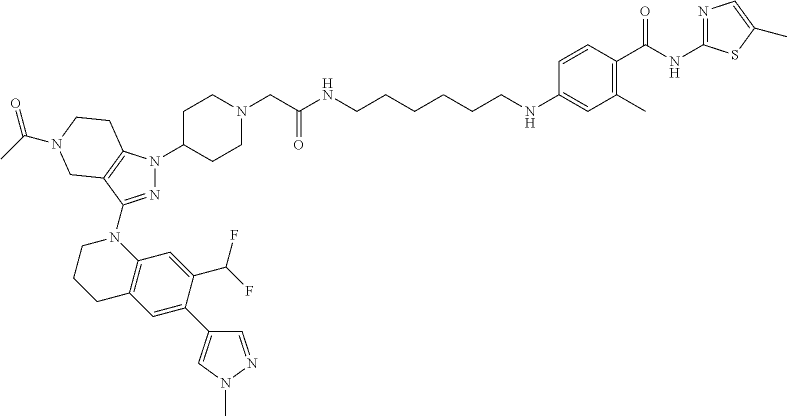 [SMILES: CC(=O)N1CCc2c(c(N3CCCc4cc(-c5cnn(C)c5)c(C(F)F)cc43)nn2C2CCN(CC(=O)NCCCCCCNc3ccc(C(=O)Nc4ncc(C)s4)c(C)c3)CC2)C1] 4-((6-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)hexyl)amino)-2-methyl-N-(5-methylthiazol-2- yl)benzamide |
| CPD-194 | 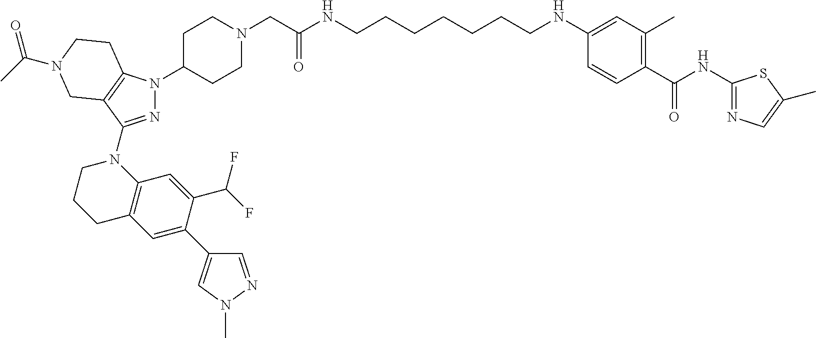 [SMILES: CC(=O)N1CCc2c(c(N3CCCc4cc(-c5cnn(C)c5)c(C(F)F)cc43)nn2C2CCN(CC(=O)NCCCCCCCNc3ccc(C(=O)Nc4ncc(C)s4)c(C)c3)CC2)C1] 4-((7-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)heptyl)amino)-2-methyl-N-(5-methylthiazol-2- yl)benzamide |
| CPD-195 | 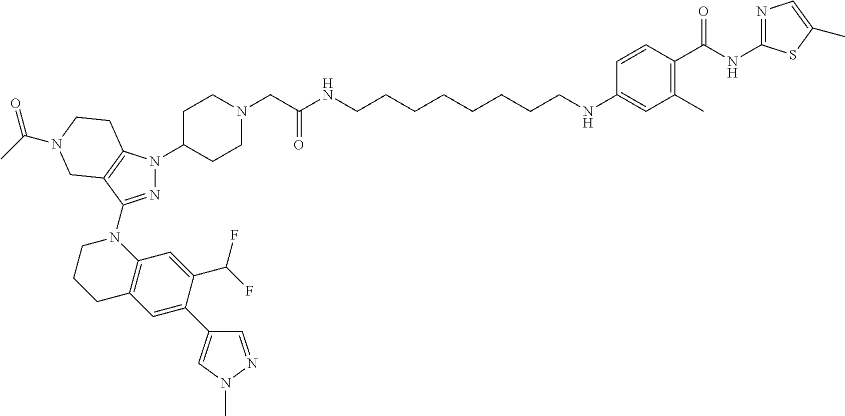 [SMILES: CC(=O)N1CCc2c(c(N3CCCc4cc(-c5cnn(C)c5)c(C(F)F)cc43)nn2C2CCN(CC(=O)NCCCCCCCCNc3ccc(C(=O)Nc4ncc(C)s4)c(C)c3)CC2)C1] 4-((8-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)octyl)amino)-2-methyl-N-(5-methylthiazol-2- yl)benzamide |
| CPD-196 | 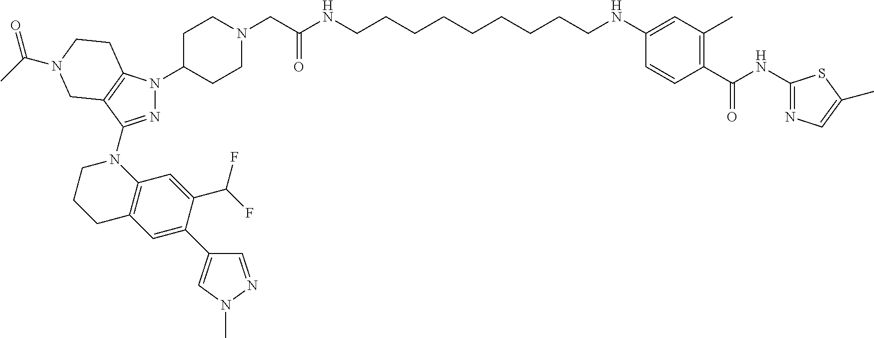 [SMILES: CC(=O)N1CCc2c(c(N3CCCc4cc(-c5cnn(C)c5)c(C(F)F)cc43)nn2C2CCN(CC(=O)NCCCCCCCCCNc3ccc(C(=O)Nc4ncc(C)s4)c(C)c3)CC2)C1] 4-((9-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)nonyl)amino)-2-methyl-N-(5-methylthiazol-2- yl)benzamide |
| CPD-197 | 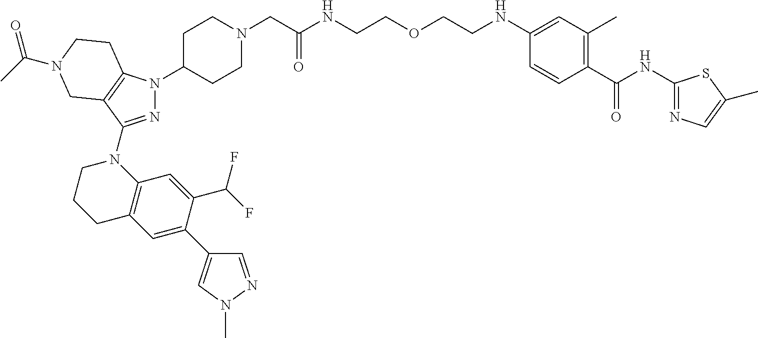 [SMILES: CC(=O)N1CCc2c(c(N3CCCc4cc(-c5cnn(C)c5)c(C(F)F)cc43)nn2C2CCN(CC(=O)NCCOCCNc3ccc(C(=O)Nc4ncc(C)s4)c(C)c3)CC2)C1] 4-((2-(2-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)ethoxy)ethyl)amino)-2-methyl-N-(5-methylthiazol-2- yl)benzamide |
| CPD-198 | 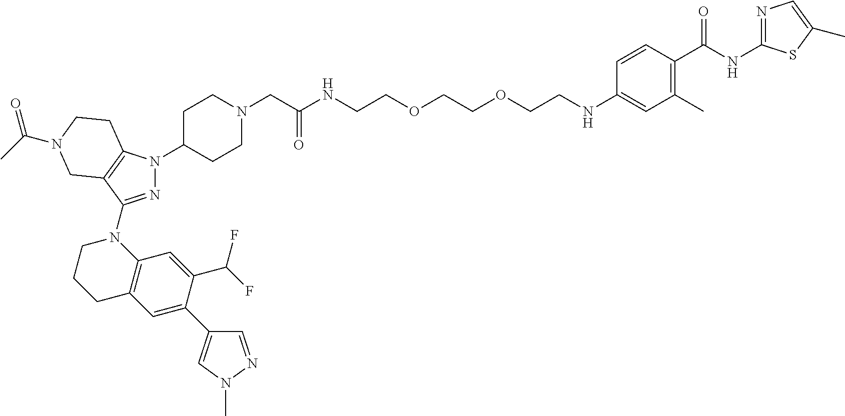 [SMILES: CC(=O)N1CCc2c(c(N3CCCc4cc(-c5cnn(C)c5)c(C(F)F)cc43)nn2C2CCN(CC(=O)NCCOCCOCCNc3ccc(C(=O)Nc4ncc(C)s4)c(C)c3)CC2)C1] 4-((2-(2-(2-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)ethoxy)ethoxy)ethyl)amino)-2-methyl-N-(5- methylthiazol-2-yl)benzamide |
| CPD-199 | 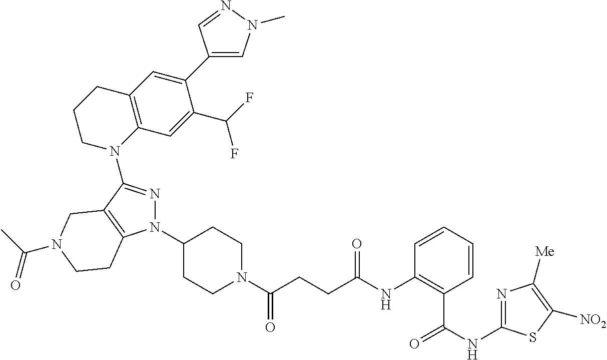 [SMILES: CC(=O)N1CCc2c(c(N3CCCc4cc(-c5cnn(C)c5)c(C(F)F)cc43)nn2C2CCN(C(=O)CCC(=O)Nc3ccccc3C(=O)Nc3nc(C)c([N+](=O)[O-])s3)CC2)C1] 2-(4-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)-4-oxobutanamido)-N-(4-methyl-5-nitrothiazol-2-yl)benzamide |
| CPD-200 | 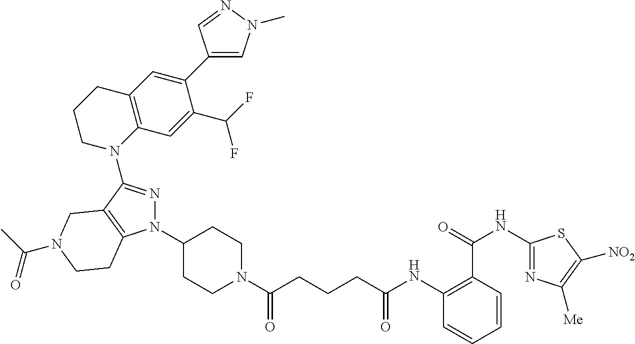 [SMILES: CC(=O)N1CCc2c(c(N3CCCc4cc(-c5cnn(C)c5)c(C(F)F)cc43)nn2C2CCN(C(=O)CCCC(=O)Nc3ccccc3C(=O)Nc3nc(C)c([N+](=O)[O-])s3)CC2)C1] 2-(5-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)-5-oxopentanamido)-N-(4-methyl-5-nitrothiazol-2-yl)benzamide |
| CPD-201 | 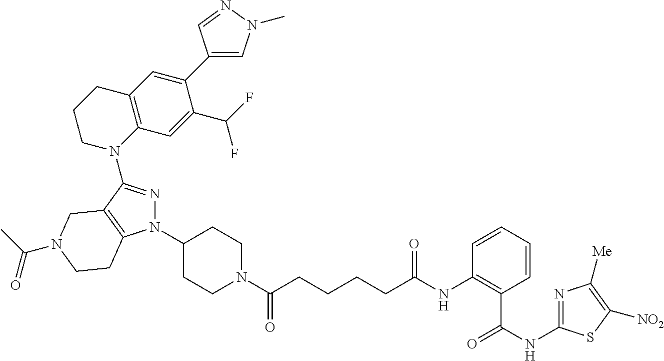 [SMILES: CC(=O)N1CCc2c(c(N3CCCc4cc(-c5cnn(C)c5)c(C(F)F)cc43)nn2C2CCN(C(=O)CCCCC(=O)Nc3ccccc3C(=O)Nc3nc(C)c([N+](=O)[O-])s3)CC2)C1] 2-(6-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)-6-oxohexanamido)-N-(4-methyl-5-nitrothiazol-2-yl)benzamide |
| CPD-202 | 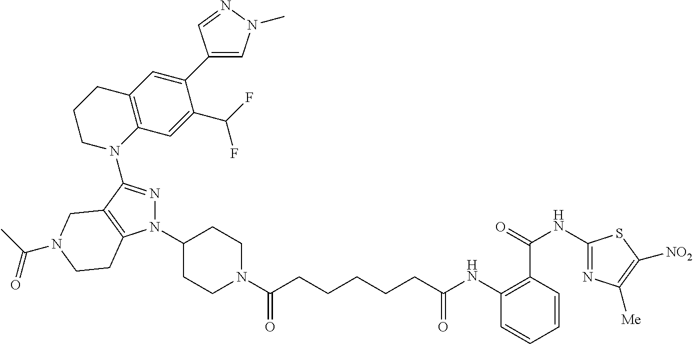 [SMILES: CC(=O)N1CCc2c(c(N3CCCc4cc(-c5cnn(C)c5)c(C(F)F)cc43)nn2C2CCN(C(=O)CCCCCC(=O)Nc3ccccc3C(=O)Nc3nc(C)c([N+](=O)[O-])s3)CC2)C1] 2-(7-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)-7-oxoheptanamido)-N-(4-methyl-5-nitrothiazol-2-yl)benzamide |
| CPD-203 | 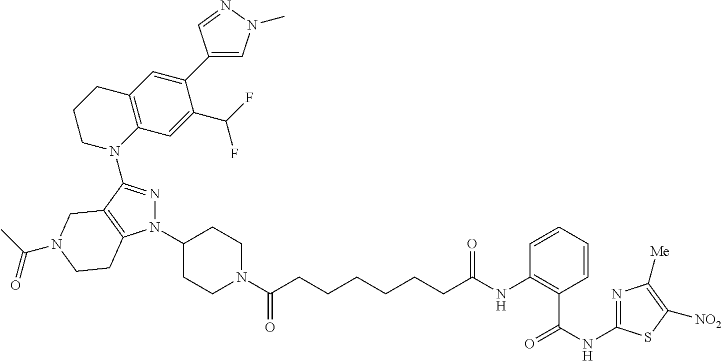 [SMILES: CC(=O)N1CCc2c(c(N3CCCc4cc(-c5cnn(C)c5)c(C(F)F)cc43)nn2C2CCN(C(=O)CCCCCCC(=O)Nc3ccccc3C(=O)Nc3nc(C)c([N+](=O)[O-])s3)CC2)C1] 2-(8-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)-8-oxooctanamido)-N-(4-methyl-5-nitrothiazol-2-yl)benzamide |
| CPD-204 | 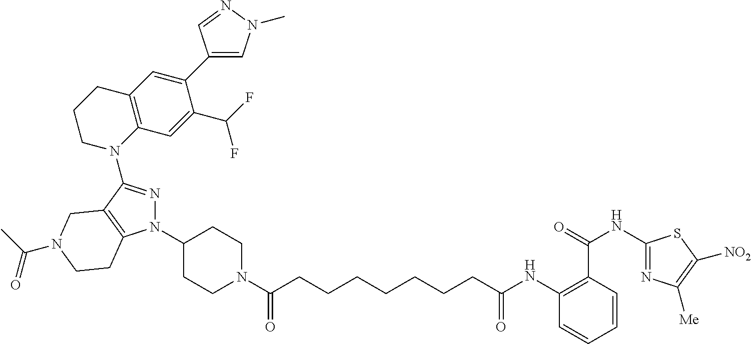 [SMILES: CC(=O)N1CCc2c(c(N3CCCc4cc(-c5cnn(C)c5)c(C(F)F)cc43)nn2C2CCN(C(=O)CCCCCCCC(=O)Nc3ccccc3C(=O)Nc3nc(C)c([N+](=O)[O-])s3)CC2)C1] 2-(9-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)-9-oxononanamido)-N-(4-methyl-5-nitrothiazol-2-yl)benzamide |
| CPD-205 | 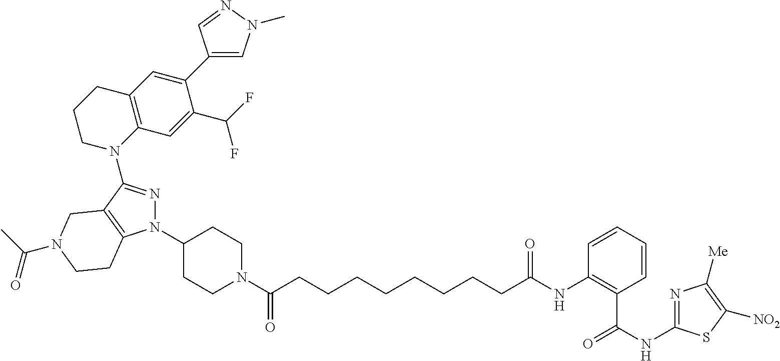 [SMILES: CC(=O)N1CCc2c(c(N3CCCc4cc(-c5cnn(C)c5)c(C(F)F)cc43)nn2C2CCN(C(=O)CCCCCCCCC(=O)Nc3ccccc3C(=O)Nc3nc(C)c([N+](=O)[O-])s3)CC2)C1] 2-(10-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)-10-oxodecanamido)-N-(4-methyl-5-nitrothiazol-2-yl)benzamide |
| CPD-206 | 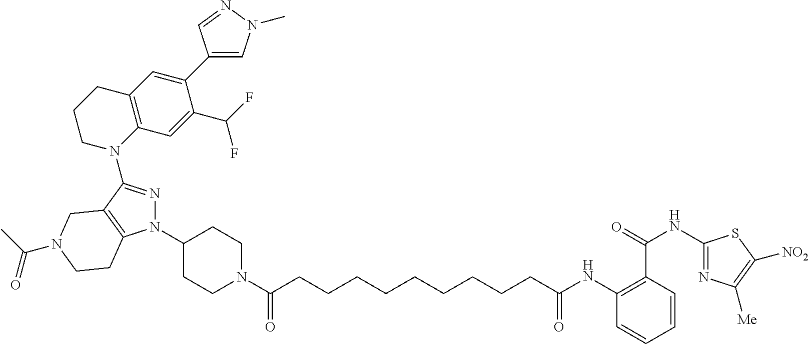 [SMILES: CC(=O)N1CCc2c(c(N3CCCc4cc(-c5cnn(C)c5)c(C(F)F)cc43)nn2C2CCN(C(=O)CCCCCCCCCC(=O)Nc3ccccc3C(=O)Nc3nc(C)c([N+](=O)[O-])s3)CC2)C1] 2-(11-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)-11-oxoundecanamido)-N-(4-methyl-5-nitrothiazol-2-yl)benzamide |
| CPD-207 | 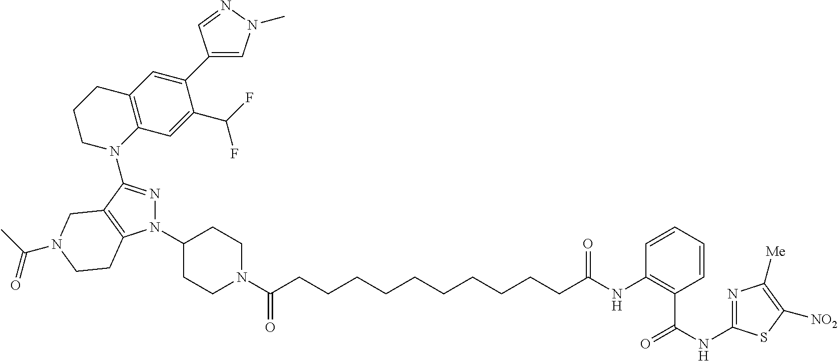 [SMILES: CC(=O)N1CCc2c(c(N3CCCc4cc(-c5cnn(C)c5)c(C(F)F)cc43)nn2C2CCN(C(=O)CCCCCCCCCCC(=O)Nc3ccccc3C(=O)Nc3nc(C)c([N+](=O)[O-])s3)CC2)C1] 2-(12-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)-12-oxododecanamido)-N-(4-methyl-5-nitrothiazol-2-yl)benzamide |
| CPD-208 | 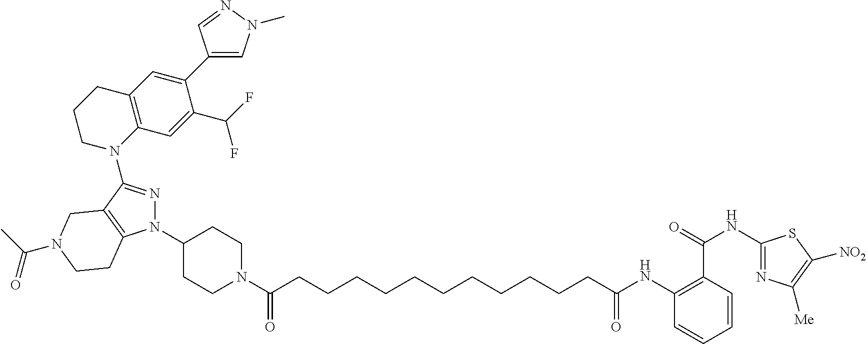 [SMILES: CC(=O)N1CCc2c(c(N3CCCc4cc(-c5cnn(C)c5)c(C(F)F)cc43)nn2C2CCN(C(=O)CCCCCCCCCCCC(=O)Nc3ccccc3C(=O)Nc3nc(C)c([N+](=O)[O-])s3)CC2)C1] 2-(13-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)-13-oxotridecanamido)-N-(4-methyl-5-nitrothiazol-2-yl)benzamide |
| CPD-209 | 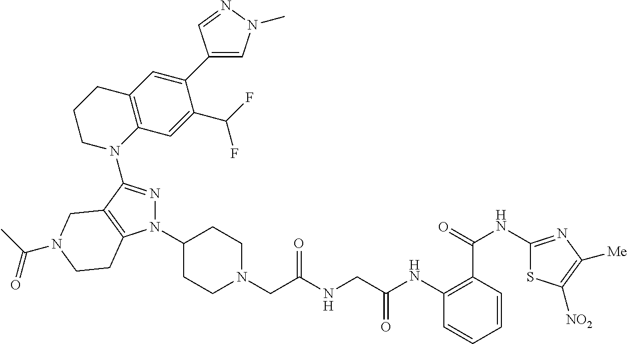 [SMILES: CC(=O)N1CCc2c(c(N3CCCc4cc(-c5cnn(C)c5)c(C(F)F)cc43)nn2C2CCN(CC(=O)NCC(=O)Nc3ccccc3C(=O)Nc3nc(C)c([N+](=O)[O-])s3)CC2)C1] 2-(2-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)acetamido)-N-(4-methyl-5-nitrothiazol-2-yl)benzamide |
| CPD-210 | 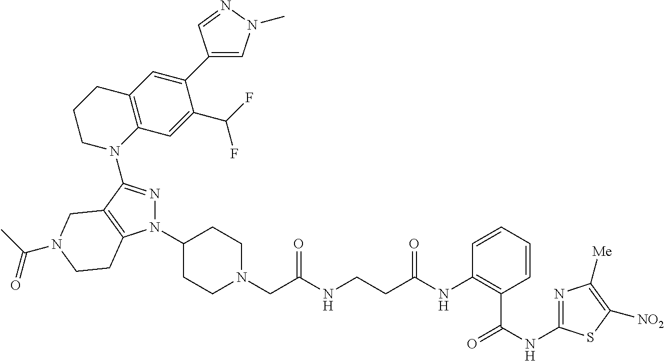 [SMILES: CC(=O)N1CCc2c(c(N3CCCc4cc(-c5cnn(C)c5)c(C(F)F)cc43)nn2C2CCN(CC(=O)NCCC(=O)Nc3ccccc3C(=O)Nc3nc(C)c([N+](=O)[O-])s3)CC2)C1] 2-(3-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)propanamido)-N-(4-methyl-5-nitrothiazol-2- yl)benzamide |
| CPD-211 | 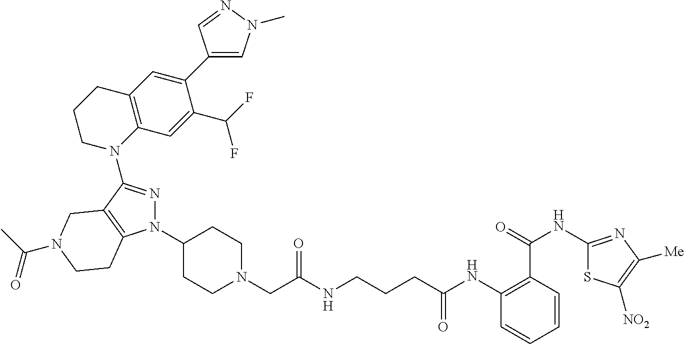 [SMILES: CC(=O)N1CCc2c(c(N3CCCc4cc(-c5cnn(C)c5)c(C(F)F)cc43)nn2C2CCN(CC(=O)NCCCC(=O)Nc3ccccc3C(=O)Nc3nc(C)c([N+](=O)[O-])s3)CC2)C1] 2-(4-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)butanamido)-N-(4-methyl-5-nitrothiazol-2-yl)benzamide |
| CPD-212 | 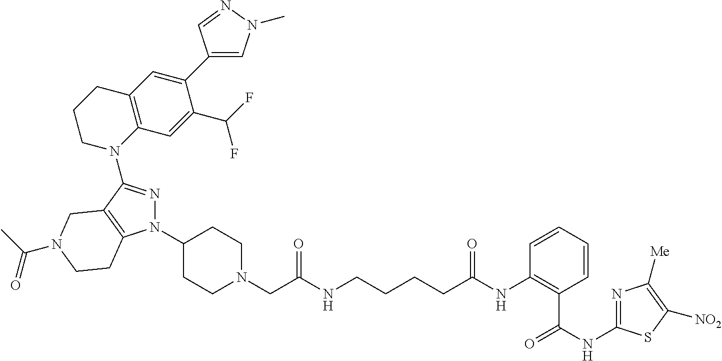 [SMILES: CC(=O)N1CCc2c(c(N3CCCc4cc(-c5cnn(C)c5)c(C(F)F)cc43)nn2C2CCN(CC(=O)NCCCCC(=O)Nc3ccccc3C(=O)Nc3nc(C)c([N+](=O)[O-])s3)CC2)C1] 2-(5-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)pentanamido)-N-(4-methyl-5-nitrothiazol-2-yl)benzamide |
| CPD-213 | 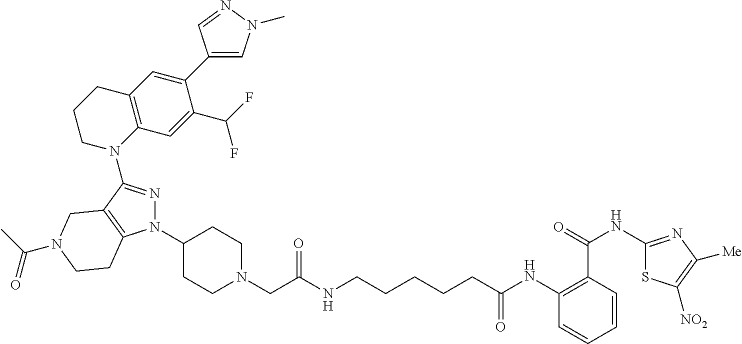 [SMILES: CC(=O)N1CCc2c(c(N3CCCc4cc(-c5cnn(C)c5)c(C(F)F)cc43)nn2C2CCN(CC(=O)NCCCCCC(=O)Nc3ccccc3C(=O)Nc3nc(C)c([N+](=O)[O-])s3)CC2)C1] 2-(6-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)hexanamido)-N-(4-methyl-5-nitrothiazol-2-yl)benzamide |
| CPD-214 | 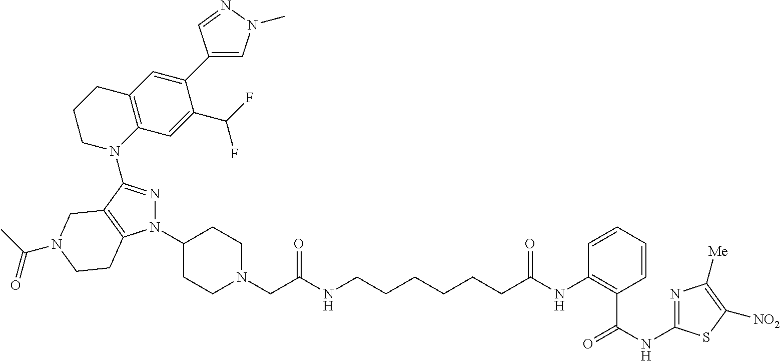 [SMILES: CC(=O)N1CCc2c(c(N3CCCc4cc(-c5cnn(C)c5)c(C(F)F)cc43)nn2C2CCN(CC(=O)NCCCCCCC(=O)Nc3ccccc3C(=O)Nc3nc(C)c([N+](=O)[O-])s3)CC2)C1] 2-(7-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)heptanamido)-N-(4-methyl-5-nitrothiazol-2-yl)benzamide |
| CPD-215 | 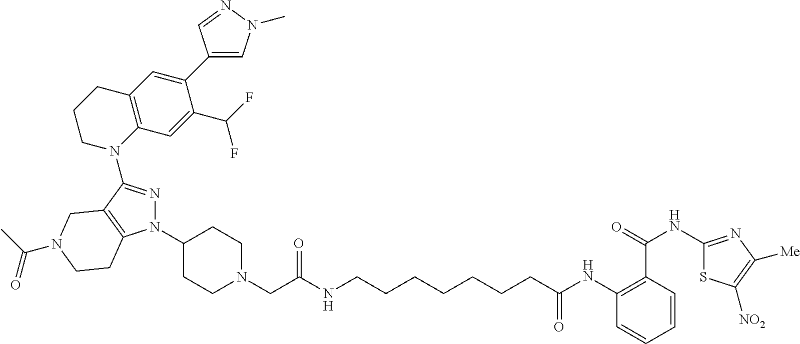 [SMILES: CC(=O)N1CCc2c(c(N3CCCc4cc(-c5cnn(C)c5)c(C(F)F)cc43)nn2C2CCN(CC(=O)NCCCCCCCC(=O)Nc3ccccc3C(=O)Nc3nc(C)c([N+](=O)[O-])s3)CC2)C1] 2-(8-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)octanamido)-N-(4-methyl-5-nitrothiazol-2-yl)benzamide |
| CPD-216 | 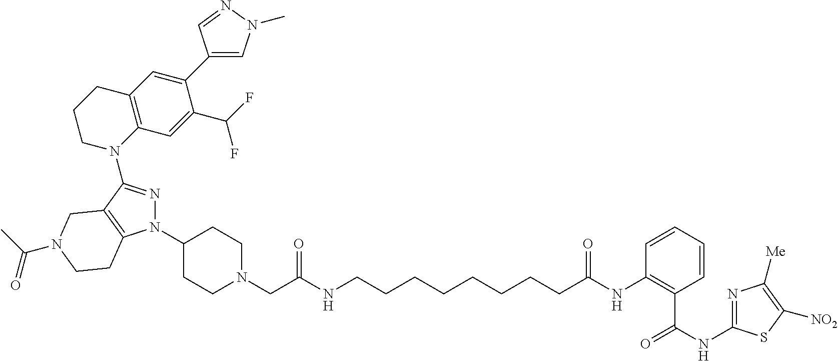 [SMILES: CC(=O)N1CCc2c(c(N3CCCc4cc(-c5cnn(C)c5)c(C(F)F)cc43)nn2C2CCN(CC(=O)NCCCCCCCCC(=O)Nc3ccccc3C(=O)Nc3nc(C)c([N+](=O)[O-])s3)CC2)C1] 2-(9-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)nonanamido)-N-(4-methyl-5-nitrothiazol-2-yl)benzamide |
| CPD-217 | 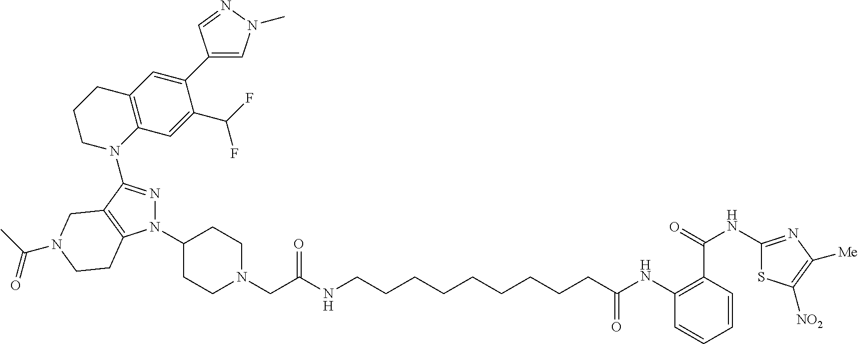 [SMILES: CC(=O)N1CCc2c(c(N3CCCc4cc(-c5cnn(C)c5)c(C(F)F)cc43)nn2C2CCN(CC(=O)NCCCCCCCCCC(=O)Nc3ccccc3C(=O)Nc3nc(C)c([N+](=O)[O-])s3)CC2)C1] 2-(10-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)decanamido)-N-(4-methyl-5-nitrothiazol-2-yl)benzamide |
| CPD-218 | 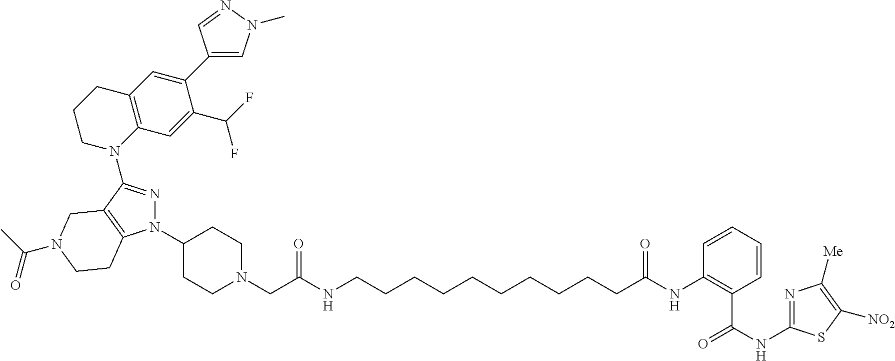 [SMILES: CC(=O)N1CCc2c(c(N3CCCc4cc(-c5cnn(C)c5)c(C(F)F)cc43)nn2C2CCN(CC(=O)NCCCCCCCCCCC(=O)Nc3ccccc3C(=O)Nc3nc(C)c([N+](=O)[O-])s3)CC2)C1] 2-(11-(2-(4-(5-acetyl-3-(7-(difluoromethyl)-6-(1-methyl-1H-pyrazol-4-yl)-3,4- dihydroquinolin-1(2H)-yl)-4,5,6,7-tetrahydro-1H-pyrazolo[4,3-c]pyridin-1- yl)piperidin-1-yl)acetamido)undecanamido)-N-(4-methyl-5-nitrothiazol-2- yl)benzamide |
| CPD-219 | 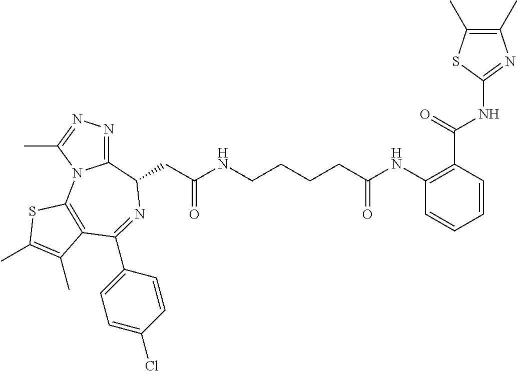 (S)-2-(5-(2-(4-(4-chlorophenyl)-2,3,9-trimethyl-6H-thieno[3,2-f][1,2,4]triazolo[4,3- a][1,4]diazepin-6-yl)acetamido)pentanamido)-N-(4,5-dimethylthiazol-2-yl)benzamide |
| CPD-220 | 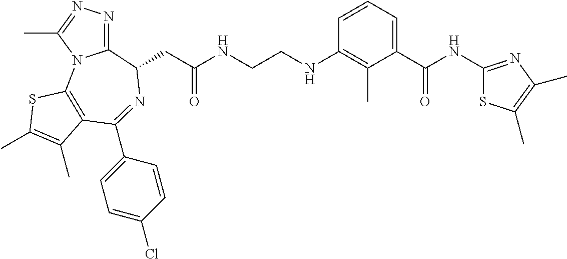 (S)-3-((2-(2-(4-(4-chlorophenyl)-2,3,9-trimethyl-6H-thieno[3,2-f][1,2,4]triazolo[4,3- a][1,4]diazepin-6-yl)acetamido)ethyl)amino)-N-(4,5-dimethylthiazol-2-yl)-2- methylbenzamide |
| CPD-221 | 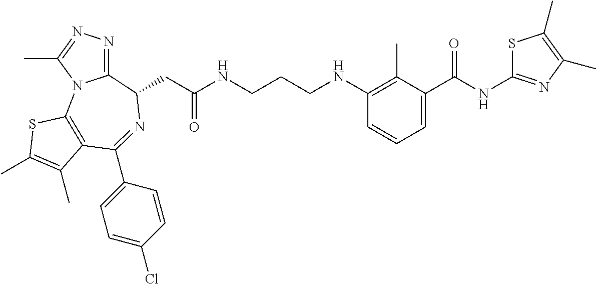 (S)-3-((3-(2-(4-(4-chlorophenyl)-2,3,9-trimethyl-6H-thieno[3,2-f][1,2,4]triazolo[4,3- a][1,4]diazepin-6-yl)acetamido)propyl)amino)-N-(4,5-dimethylthiazol-2-yl)-2- methylbenzamide |
| CPD-222 | 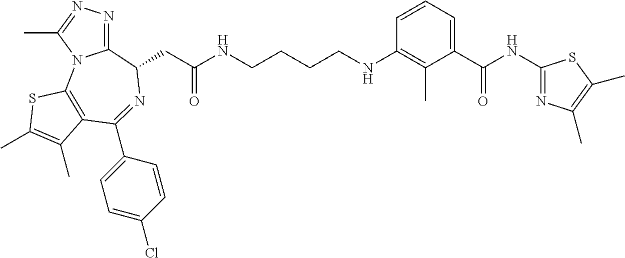 (S)-3-((4-(2-(4-(4-chlorophenyl)-2,3,9-trimethyl-6H-thieno[3,2-f][1,2,4]triazolo[4,3- a][1,4]diazepin-6-yl)acetamido)butyl)amino)-N-(4,5-dimethylthiazol-2-yl)-2- methylbenzamide |
| CPD-223 | 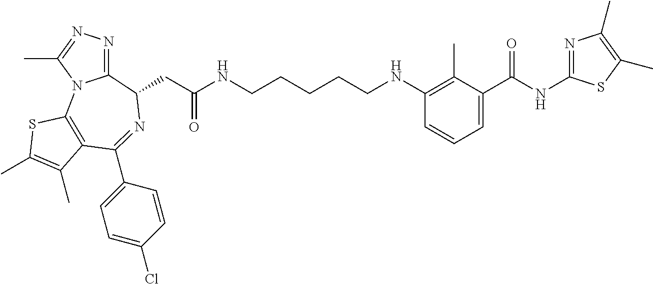 (S)-3-((5-(2-(4-(4-chlorophenyl)-2,3,9-trimethyl-6H-thieno[3,2-f][1,2,4]triazolo[4,3- a][1,4]diazepin-6-yl)acetamido)pentyl)amino)-N-(4,5-dimethylthiazol-2-yl)-2- methylbenzamide |
| CPD-224 | 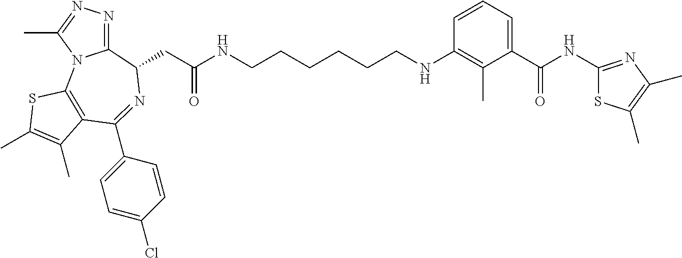 (S)-3-((6-(2-(4-(4-chlorophenyl)-2,3,9-trimethyl-6H-thieno[3,2-f][1,2,4]triazolo[4,3- a][1,4]diazepin-6-yl)acetamido)hexyl)amino)-N-(4,5-dimethylthiazol-2-yl)-2- methylbenzamide |
| CPD-225 | 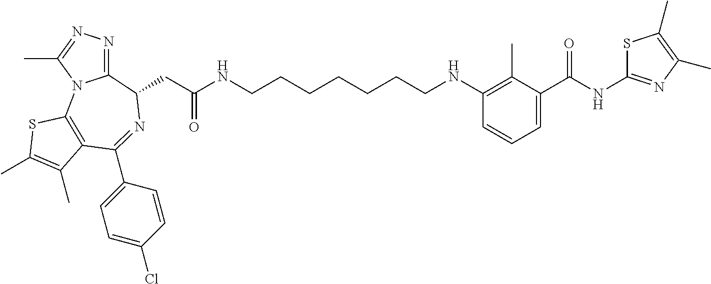 (S)-3-((7-(2-(4-(4-chlorophenyl)-2,3,9-trimethyl-6H-thieno[3,2-f][1,2,4]triazolo[4,3- a][1,4]diazepin-6-yl)acetamido)heptyl)amino)-N-(4,5-dimethylthiazol-2-yl)-2- methylbenzamide |
| CPD-226 | 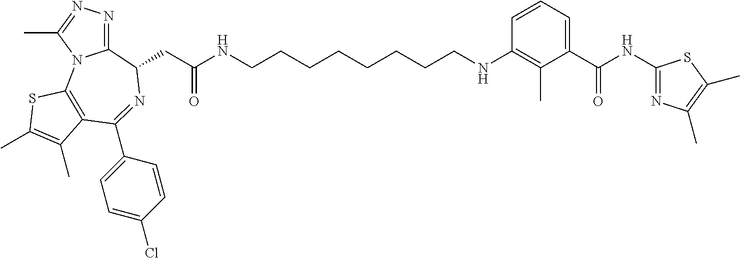 (S)-3-((8-(2-(4-(4-chlorophenyl)-2,3,9-trimethyl-6H-thieno[3,2-f][1,2,4]triazolo[4,3- a][1,4]diazepin-6-yl)acetamido)octyl)amino)-N-(4,5-dimethylthiazol-2-yl)-2- methylbenzamide |
| CPD-227 | 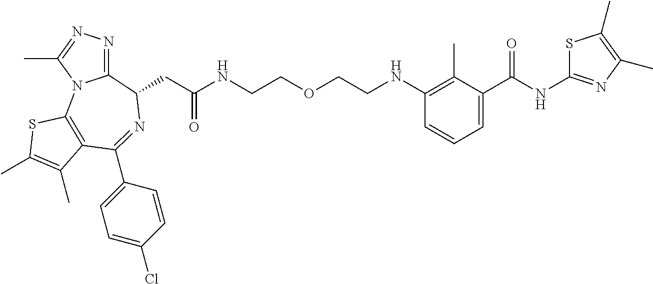 (S)-3-((2-(2-(2-(4-(4-chlorophenyl)-2,3,9-trimethyl-6H-thieno[3,2- f][1,2,4]triazolo[4,3-a][1,4]diazepin-6-yl)acetamido)ethoxy)ethyl)amino)-N-(4,5- dimethylthiazol-2-yl)-2-methylbenzamide |
| CPD-228 | 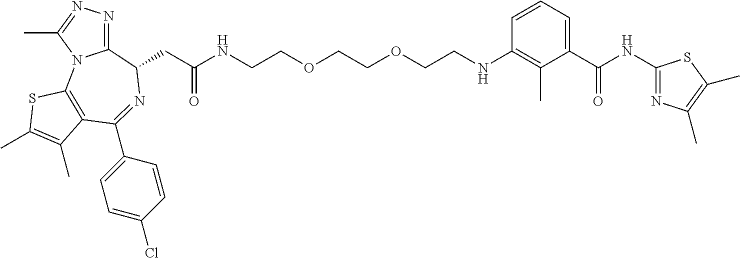 (S)-3-((2-(2-(2-(2-(4-(4-chlorophenyl)-2,3,9-trimethyl-6H-thieno[3,2- f][1,2,4]triazolo[4,3-a][1,4]diazepin-6-yl)acetamido)ethoxy)ethoxy)ethyl)amino)-N- (4,5-dimethylthiazol-2-yl)-2-methylbenzamide |
| CPD-229 | 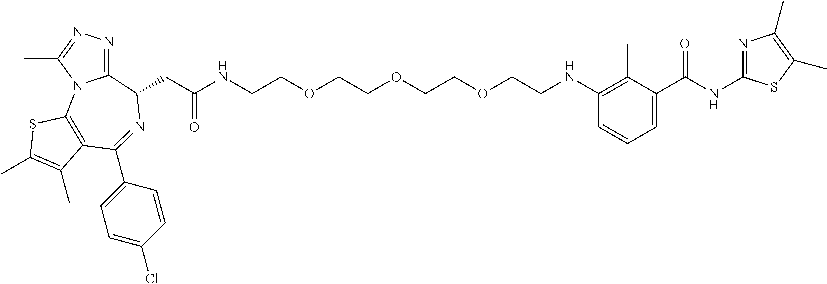 (S)-3-((1-(4-(4-chlorophenyl)-2,3,9-trimethyl-6H-thieno[3,2-f][1,2,4]triazolo[4,3- a][1,4]diazepin-6-yl)-2-oxo-6,9,12-trioxa-3-azatetradecan-14-yl)amino)-N-(4,5- dimethylthiazol-2-yl)-2-methylbenzamide |
| CPD-230 | 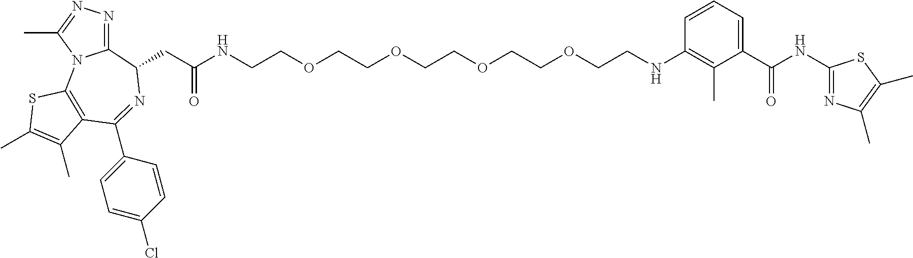 (S)-3-((1-(4-(4-chlorophenyl)-2,3,9-trimethyl-6H-thieno[3,2-f][1,2,4]triazolo[4,3- a][1,4]diazepin-6-yl)-2-oxo-6,9,12,15-tetraoxa-3-azaheptadecan-17-yl)amino)-N-(4,5- dimethylthiazol-2-yl)-2-methylbenzamide |
| CPD-231 | 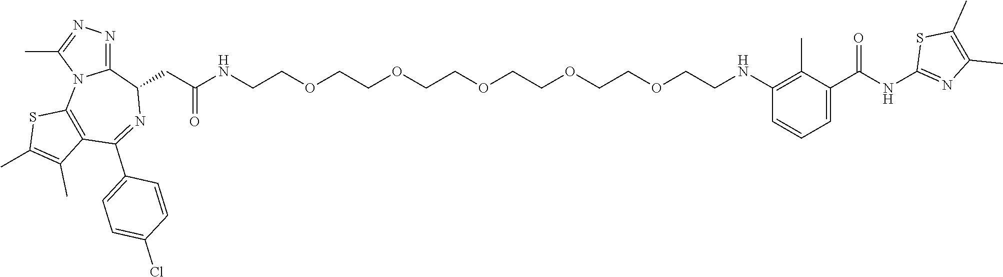 (S)-3-((1-(4-(4-chlorophenyl)-2,3,9-trimethyl-6H-thieno[3,2-f][1,2,4]triazolo[4,3- a][1,4]diazepin-6-yl)-2-oxo-6,9,12,15,18-pentaoxa-3-azaicosan-20-yl)amino)-N-(4,5- dimethylthiazol-2-yl)-2-methylbenzamide |
| CPD-232 | 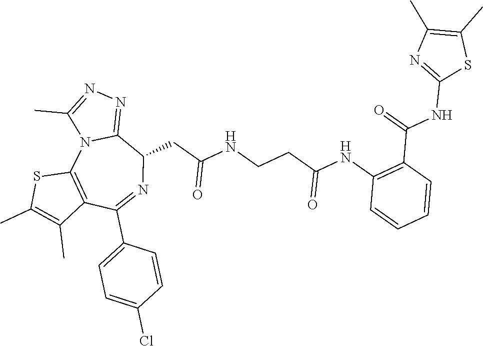 (S)-2-(3-(2-(4-(4-chlorophenyl)-2,3,9-trimethyl-6H-thieno[3,2-f][1,2,4]triazolo[4,3- a][1,4]diazepin-6-yl)acetamido)propanamido)-N-(4,5-dimethylthiazol-2-yl)benzamide |
| CPD-233 | 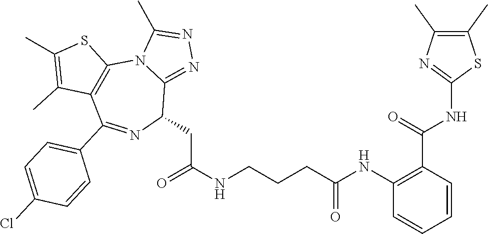 [SMILES: Cc1nc(NC(=O)c2ccccc2NC(=O)CCCNC(=O)C[C@@H]2N=C(c3ccc(Cl)cc3)c3c(sc(C)c3C)-n3c(C)nnc32)sc1C] (S)-2-(4-(2-(4-(4-chlorophenyl)-2,3,9-trimethyl-6H-thieno[3,2-f][1,2,4]triazolo[4,3- a][1,4]diazepin-6-yl)acetamido)butanamido)-N-(4,5-dimethylthiazol-2-yl)benzamide |
| CPD-234 | 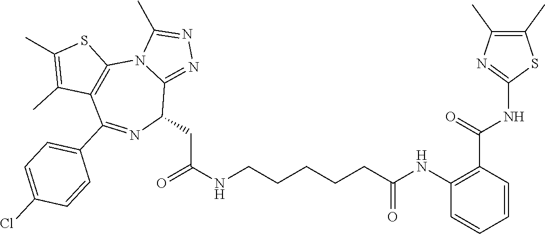 [SMILES: Cc1nc(NC(=O)c2ccccc2NC(=O)CCCCCNC(=O)C[C@@H]2N=C(c3ccc(Cl)cc3)c3c(sc(C)c3C)-n3c(C)nnc32)sc1C] (S)-2-(6-(2-(4-(4-chlorophenyl)-2,3,9-trimethyl-6H-thieno[3,2-f][1,2,4]triazolo[4,3- a][1,4]diazepin-6-yl)acetamido)hexanamido)-N-(4,5-dimethylthiazol-2-yl)benzamide |
| CPD-235 | 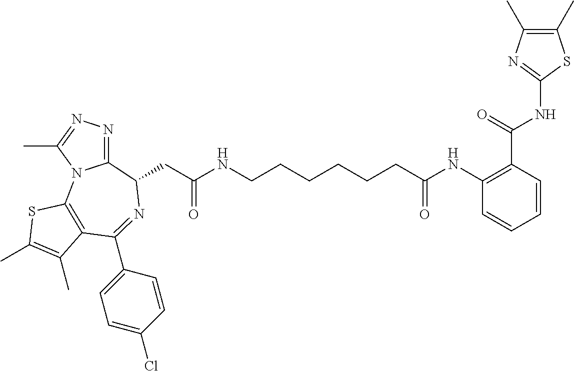 (S)-2-(7-(2-(4-(4-chlorophenyl)-2,3,9-trimethyl-6H-thieno[3,2-f][1,2,4]triazolo[4,3- a][1,4]diazepin-6-yl)acetamido)heptanamido)-N-(4,5-dimethylthiazol-2-yl)benzamide |
| CPD-236 | 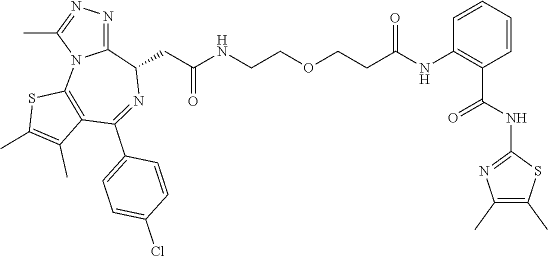 (S)-2-(3-(2-(2-(4-(4-chlorophenyl)-2,3,9-trimethyl-6H-thieno[3,2-f][1,2,4]triazolo[4,3- a][1,4]diazepin-6-yl)acetamido)ethoxy)propanamido)-N-(4,5-dimethylthiazol-2- yl)benzamide |
| CPD-237 | 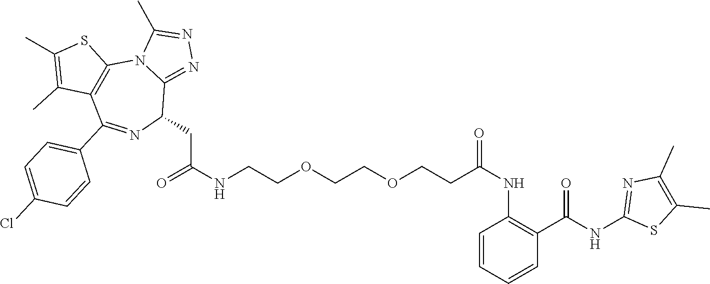 [SMILES: Cc1nc(NC(=O)c2ccccc2NC(=O)CCOCCOCCNC(=O)C[C@@H]2N=C(c3ccc(Cl)cc3)c3c(sc(C)c3C)-n3c(C)nnc32)sc1C] (S)-2-(3-(2-(2-(2-(4-(4-chlorophenyl)-2,3,9-trimethyl-6H-thieno[3,2- f][1,2,4]triazolo[4,3-a][1,4]diazepin-6-yl)acetamido)ethoxy)ethoxy)propanamido)-N- (4,5-dimethylthiazol-2-yl)benzamide |
| CPD-238 | 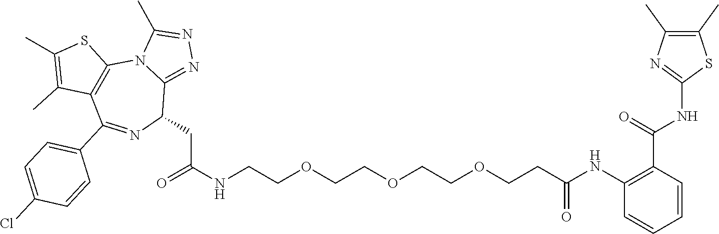 [SMILES: Cc1nc(NC(=O)c2ccccc2NC(=O)CCOCCOCCOCCNC(=O)C[C@@H]2N=C(c3ccc(Cl)cc3)c3c(sc(C)c3C)-n3c(C)nnc32)sc1C] (S)-2-(1-(4-(4-chlorophenyl)-2,3,9-trimethyl-6H-thieno[3,2-f] [1,2,4]triazolo[4,3- a][1,4]diazepin-6-yl)-2-oxo-6,9,12-trioxa-3-azapentadecan-15-amido)-N-(4,5- dimethylthiazol-2-yl)benzamide |
| CPD-239 | 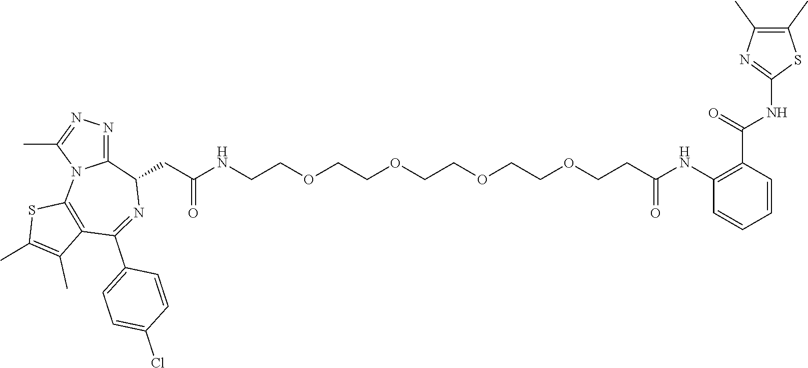 (S)-2-(1-(4-(4-chlorophenyl)-2,3,9-trimethyl-6H-thieno[3,2-f][1,2,4]triazolo[4,3- a][1,4]diazepin-6-yl)-2-oxo-6,9,12,15-tetraoxa-3-azaoctadecan-18-amido)-N-(4,5- dimethylthiazol-2-yl)benzamide |
| CPD-240 | 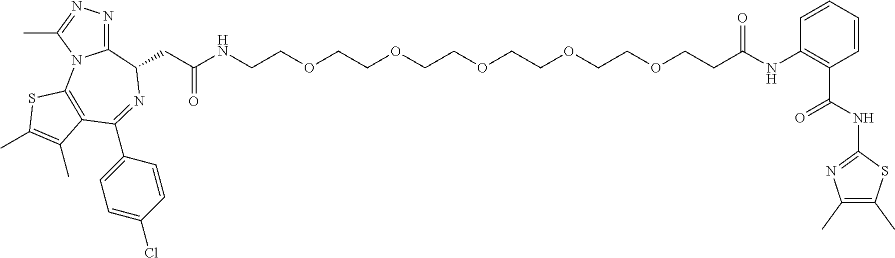 (S)-2-(1-(4-(4-chlorophenyl)-2,3,9-trimethyl-6H-thieno[3,2-f][1,2,4]triazolo[4,3- a][1,4]diazepin-6-yl)-2-oxo-6,9,12,15,18-pentaoxa-3-azahenicosan-21-amido)-N-(4,5- dimethylthiazol-2-yl)benzamide |
| CPD-241 | 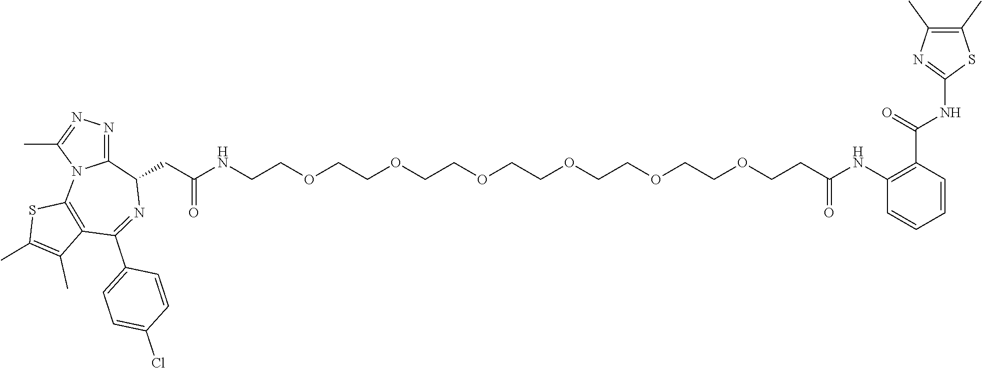 (S)-2-(1-(4-(4-chlorophenyl)-2,3,9-trimethyl-6H-thieno[3,2-f][1,2,4]triazolo[4,3- a][1,4]diazepin-6-yl)-2-oxo-6,9,12,15,18,21-hexaoxa-3-azatetracosan-24-amido)-N- (4,5-dimethylthiazol-2-yl)benzamide |
| CPD-242 | 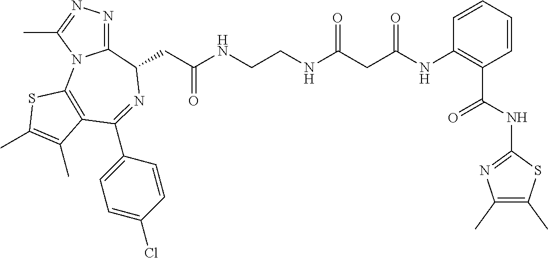 (S)-N¹-(2-(2-(4-(4-chlorophenyl)-2,3,9-trimethyl-6H-thieno[3,2-f][1,2,4]triazolo[4,3- a][1,4]diazepin-6-yl)acetamido)ethyl)-N³-(2-((4,5-dimethylthiazol-2- yl)carbamoyl)phenyl)malonamide |
| CPD-243 | 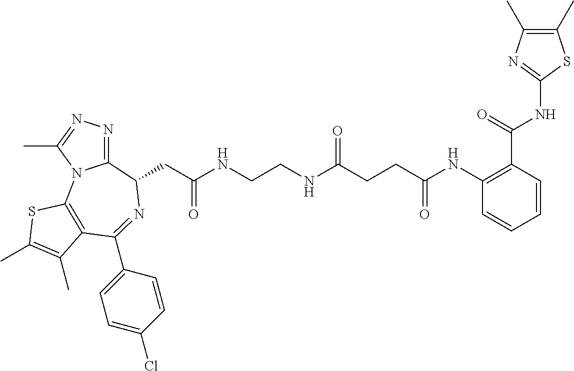 (S)-N¹-(2-(2-(4-(4-chlorophenyl)-2,3,9-trimethyl-6H-thieno[3,2-f][1,2,4]triazolo[4,3- a][1,4]diazepin-6-yl)acetamido)ethyl)-N⁴-(2-((4,5-dimethylthiazol-2- yl)carbamoyl)phenyl)succinamide |
| CPD-244 | 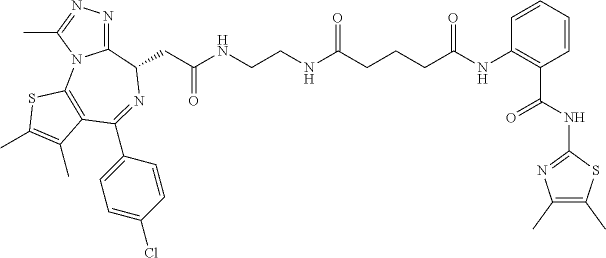 (S)-N¹-(2-(2-(4-(4-chlorophenyl)-2,3,9-trimethyl-6H-thieno[3,2-f][1,2,4]triazolo[4,3- a][1,4]diazepin-6-yl)acetamido)ethyl)-N⁵-(2-((4,5-dimethylthiazol-2- yl)carbamoyl)phenyl)glutaramide |
| CPD-245 | 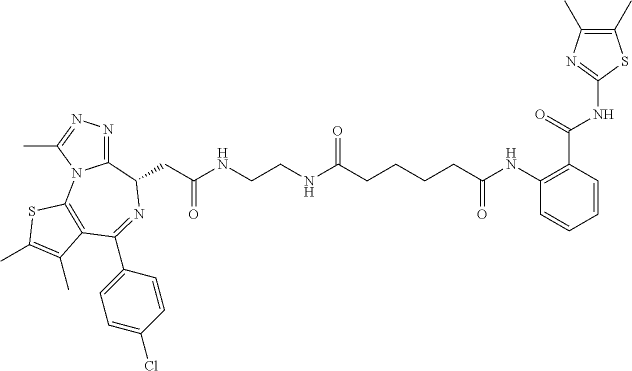 (S)-N¹-(2-(2-(4-(4-chlorophenyl)-2,3,9-trimethyl-6H-thieno[3,2-f][1,2,4]triazolo[4,3- a][1,4]diazepin-6-yl)acetamido)ethyl)-N⁶-(2-((4,5-dimethylthiazol-2- yl)carbamoyl)phenyl)adipamide |
| CPD-246 | 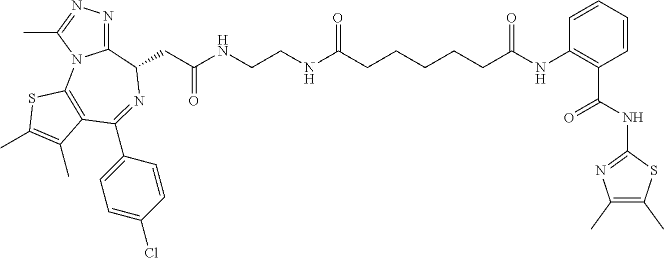 (S)-N¹-(2-(2-(4-(4-chlorophenyl)-2,3,9-trimethyl-6H-thieno[3,2-f][1,2,4]triazolo[4,3- a][1,4]diazepin-6-yl)acetamido)ethyl)-N⁷-(2-((4,5-dimethylthiazol-2- yl)carbamoyl)phenyl)heptanediamide |
| CPD-247 | 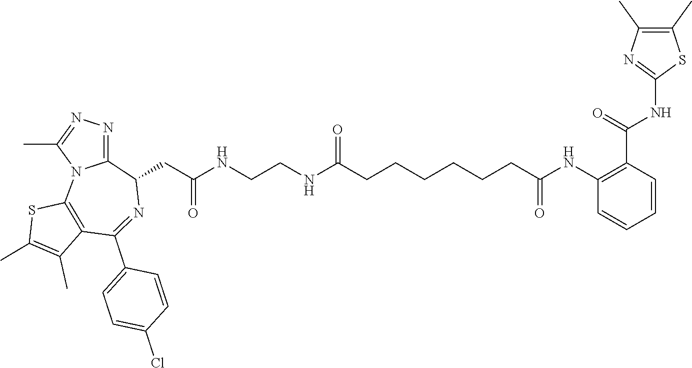 (S)-N¹-(2-(2-(4-(4-chlorophenyl)-2,3,9-trimethyl-6H-thieno[3,2-f][1,2,4]triazolo[4,3- a][1,4]diazepin-6-yl)acetamido)ethyl)-N⁸-(2-((4,5-dimethylthiazol-2- yl)carbamoyl)phenyl)octanediamide |
| CPD-248 | 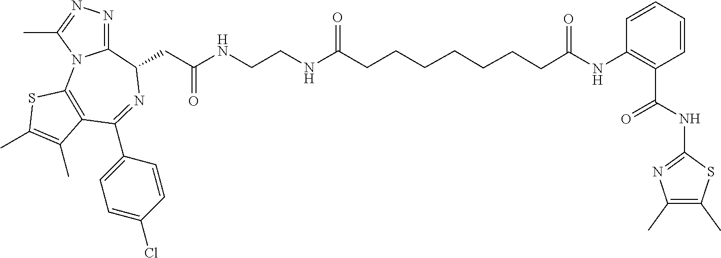 (S)-N¹-(2-(2-(4-(4-chlorophenyl)-2,3,9-trimethyl-6H-thieno[3,2-f][1,2,4]triazolo[4,3- a][1,4]diazepin-6-yl)acetamido)ethyl)-N⁹-(2-((4,5-dimethylthiazol-2- yl)carbamoyl)phenyl)nonanediamide |
| CPD-249 | 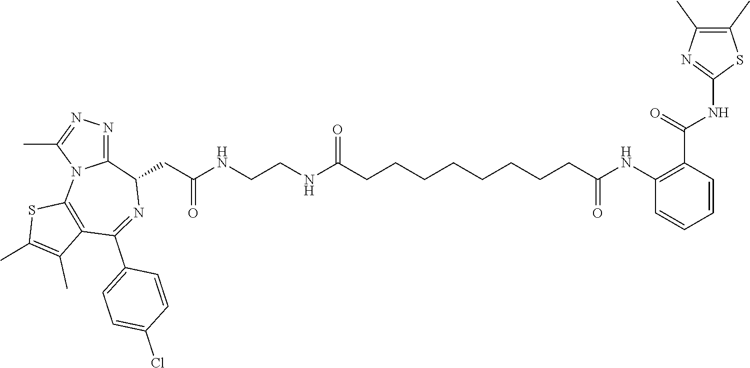 (S)-N¹-(2-(2-(4-(4-chlorophenyl)-2,3,9-trimethyl-6H-thieno[3,2-f][1,2,4]triazolo[4,3- a][1,4]diazepin-6-yl)acetamido)ethyl)-N¹⁰-(2-((4,5-dimethylthiazol-2- yl)carbamoyl)phenyl)decanediamide |
| CPD-250 | 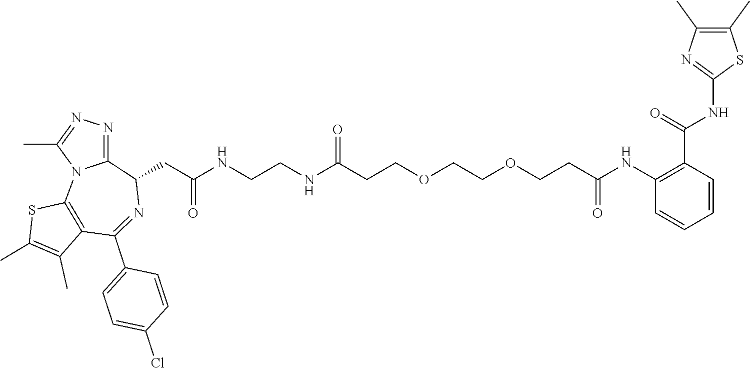 (S)-2-(1-(4-(4-chlorophenyl)-2,3,9-trimethyl-6H-thieno[3,2-f][1,2,4]triazolo[4,3- a][1,4]diazepin-6-yl)-2,7-dioxo-10,13-dioxa-3,6-diazahexadecan-16-amido)-N-(4,5- dimethylthiazol-2-yl)benzamide |
| CPD-251 | 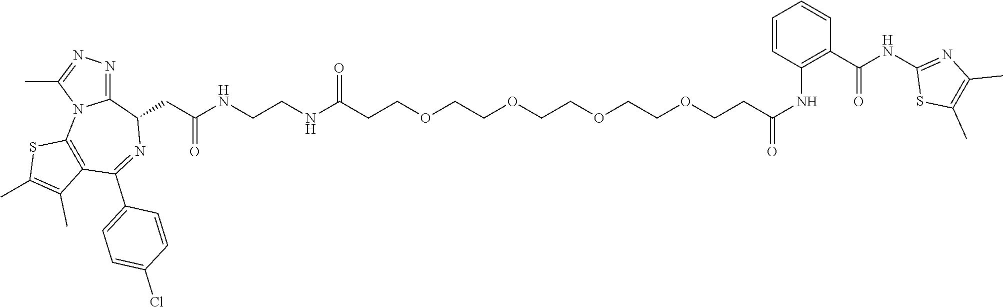 (S)-N¹-(2-(2-(4-(4-chlorophenyl)-2,3,9-trimethyl-6H-thieno[3,2-f][1,2,4]triazolo[4,3- a][1,4]diazepin-6-yl)acetamido)ethyl)-N¹⁶-(2-((4,5-dimethylthiazol-2- yl)carbamoyl)phenyl)-4,7,10,13-tetraoxahexadecanediamide |
| CPD-252 | 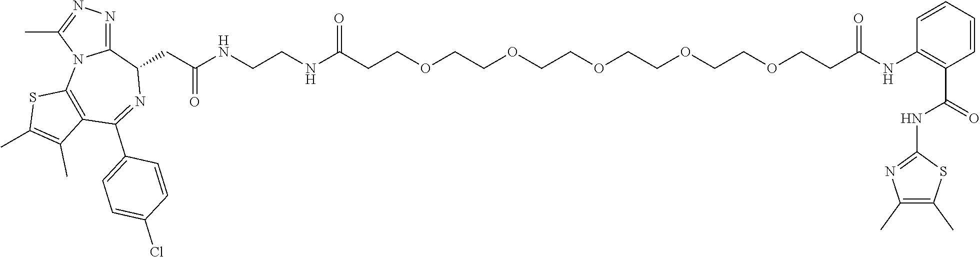 [SMILES: Cc1nc(NC(=O)c2ccccc2NC(=O)CCOCCOCCOCCOCCOCCC(=O)NCCNC(=O)C[C@@H]2N=C(c3ccc(Cl)cc3)c3c(sc(C)c3C)-n3c(C)nnc32)sc1C] (S)-N¹-(2-(2-(4-(4-chlorophenyl)-2,3,9-trimethyl-6H-thieno[3,2-f][1,2,4]triazolo[4,3- a][1,4]diazepin-6-yl)acetamido)ethyl)-N¹⁹-(2-((4,5-dimethylthiazol-2- yl)carbamoyl)phenyl)-4,7,10,13,16-pentaoxanonadecanediamide |
| CPD-253 | 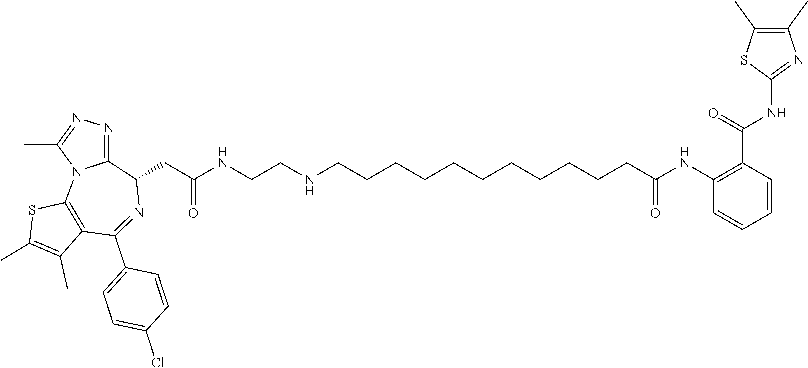 (S)-2-(12-((2-(2-(4-(4-chlorophenyl)-2,3,9-trimethyl-6H-thieno[3,2- f][1,2,4]triazolo[4,3-a][1,4]diazepin-6-yl)acetamido)ethyl)amino)dodecanamido)-N- (4,5-dimethylthiazol-2-yl)benzamide |
| CPD-254 | 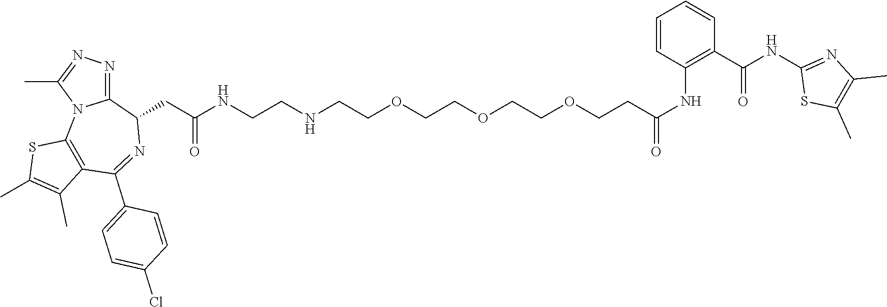 (S)-2-(1-(4-(4-chlorophenyl)-2,3,9-trimethyl-6H-thieno[3,2-f][1,2,4]triazolo[4,3- a][1,4]diazepin-6-yl)-2-oxo-9,12,15-trioxa-3,6-diazaoctadecan-18-amido)-N-(4,5- dimethylthiazol-2-yl)benzamide |
| CPD-255 | 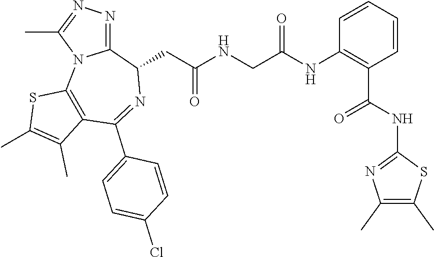 (S)-2-(2-(2-(4-(4-chlorophenyl)-2,3,9-trimethyl-6H-thieno[3,2-f][1,2,4]triazolo[4,3- a][1,4]diazepin-6-yl)acetamido)acetamido)-N-(4,5-dimethylthiazol-2-yl)benzamide |
| CPD-256 | 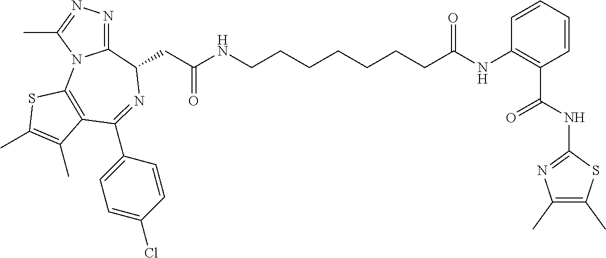 (S)-2-(8-(2-(4-(4-chlorophenyl)-2,3,9-trimethyl-6H-thieno[3,2-f][1,2,4]triazolo[4,3- a][1,4]diazepin-6-yl)acetamido)octanamido)-N-(4,5-dimethylthiazol-2-yl)benzamide |
| CPD-257 | 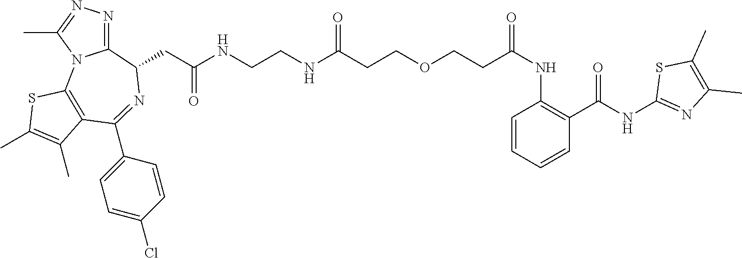 (S)-2-(3-(3-((2-(2-(4-(4-chlorophenyl)-2,3,9-trimethyl-6H-thieno[3,2- f][1,2,4]triazolo[4,3-a][1,4]diazepin-6-yl)acetamido)ethyl)amino)-3- oxopropoxy)propanamido)-N-(4,5-dimethylthiazol-2-yl)benzamide |
| CPD-258 | 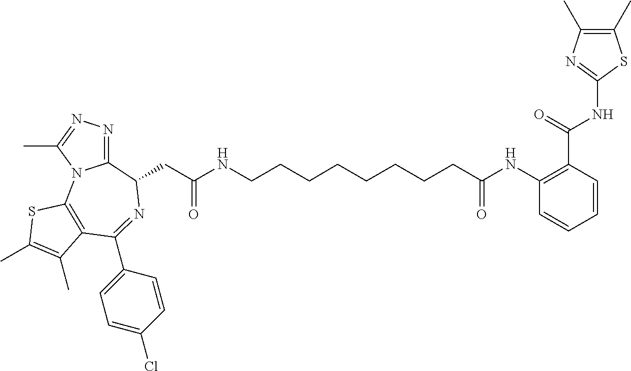 (S)-2-(9-(2-(4-(4-chlorophenyl)-2,3,9-trimethyl-6H-thieno[3,2-f][1,2,4]triazolo[4,3- a][1,4]diazepin-6-yl)acetamido)nonanamido)-N-(4,5-dimethylthiazol-2-yl)benzamide |
| CPD-259 | 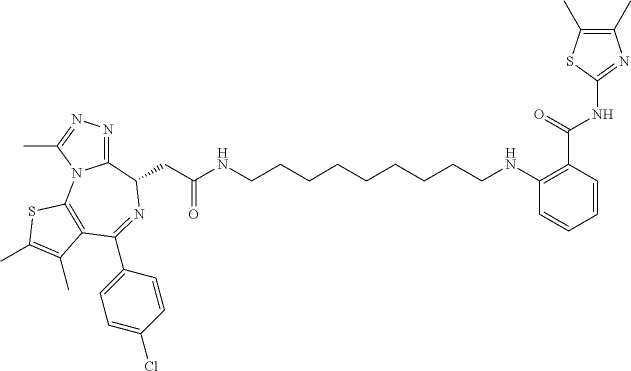 (S)-2-((9-(2-(4-(4-chlorophenyl)-2,3,9-trimethyl-6H-thieno[3,2-f][1,2,4]triazolo[4,3- a][1,4]diazepin-6-yl)acetamido)nonyl)amino)-N-(4,5-dimethylthiazol-2-yl)benzamide |
| CPD-260 | 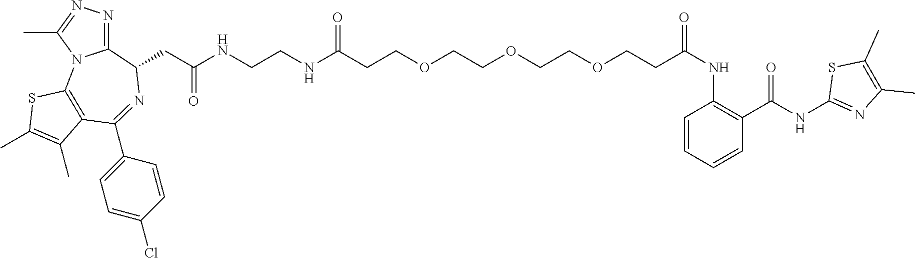 (S)-2-(1-(4-(4-chlorophenyl)-2,3,9-trimethyl-6H-thieno[3,2-f][1,2,4]triazolo[4,3- a][1,4]diazepin-6-yl)-2,7-dioxo-10,13,16-trioxa-3,6-diazanonadecan-19-amido)-N- (4,5-dimethylthiazol-2-yl)benzamide |
| CPD-261 | 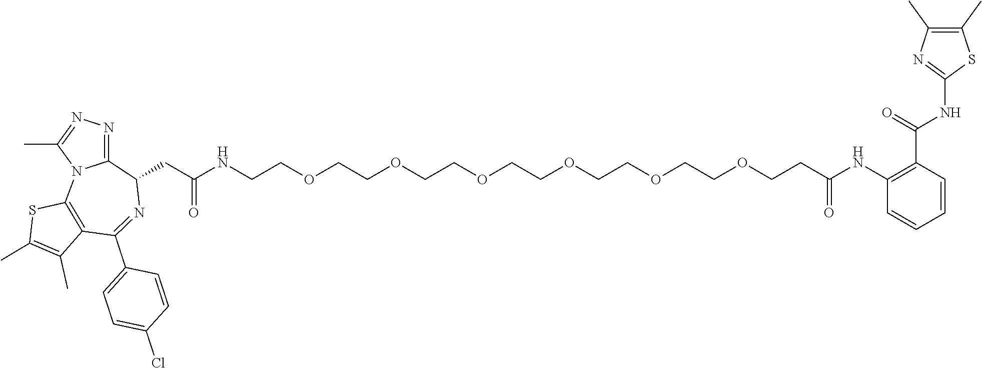 (S)-2-(1-(4-(4-chlorophenyl)-2,3,9-trimethyl-6H-thieno[3,2-f][1,2,4]triazolo[4,3- a][1,4]diazepin-6-yl)-2-oxo-6,9,12,15,18,21-hexaoxa-3-azatetracosan-24-amido)-N- (4,5-dimethylthiazol-2-yl)benzamide |
| CPD-262 | 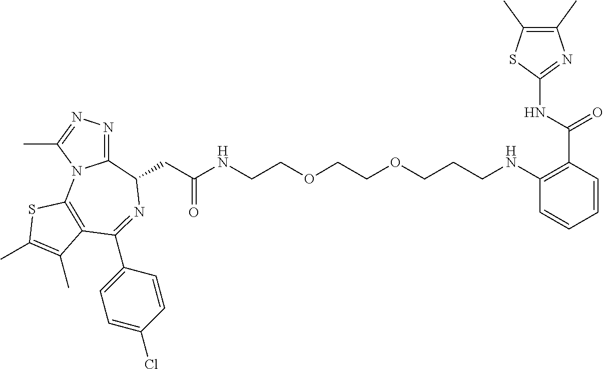 [SMILES: Cc1nc(NC(=O)c2ccccc2NCCCOCCOCCNC(=O)C[C@@H]2N=C(c3ccc(Cl)cc3)c3c(sc(C)c3C)-n3c(C)nnc32)sc1C] (S)-2-((3-(2-(2-(2-(4-(4-chlorophenyl)-2,3,9-trimethyl-6H-thieno[3,2- f][1,2,4]triazolo[4,3-a][1,4]diazepin-6-yl)acetamido)ethoxy)ethoxy)propyl)amino)-N- (4,5-dimethylthiazol-2-yl)benzamide |
| CPD-263 | 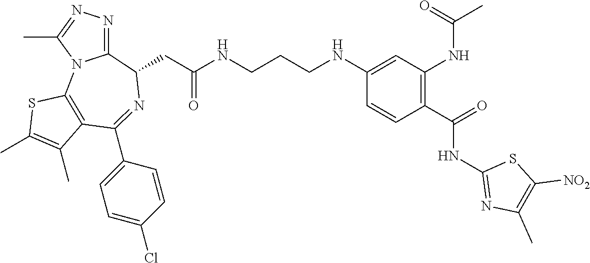 [SMILES: CC(=O)Nc1cc(NCCCNC(=O)C[C@@H]2N=C(c3ccc(Cl)cc3)c3c(sc(C)c3C)-n3c(C)nnc32)ccc1C(=O)Nc1nc(C)c([N+](=O)[O-])s1] (S)-2-acetamido-4-((3-(2-(4-(4-chlorophenyl)-2,3,9-trimethyl-6H-thieno[3,2- f][1,2,4]triazolo[4,3-a][1,4]diazepin-6-yl)acetamido)propyl)amino)-N-(4-methyl-5- nitrothiazol-2-yl)benzamide |
| CPD-264 | 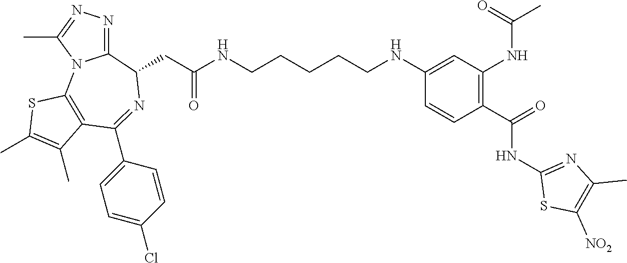 (S)-2-acetamido-4-((5-(2-(4-(4-chlorophenyl)-2,3,9-trimethyl-6H-thieno[3,2- f][1,2,4]triazolo[4,3-a][1,4]diazepin-6-yl)acetamido)pentyl)amino)-N-(4-methyl-5- nitrothiazol-2-yl)benzamide |
| CPD-265 | 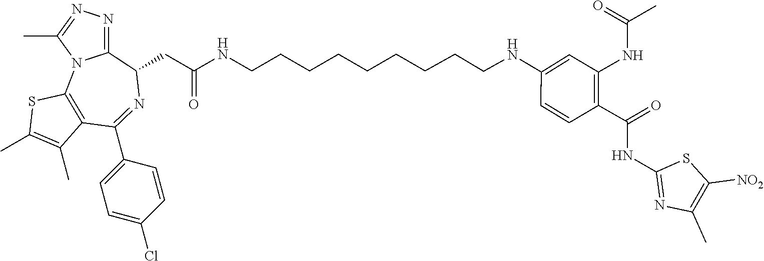 [SMILES: CC(=O)Nc1cc(NCCCCCCCCCNC(=O)C[C@@H]2N=C(c3ccc(Cl)cc3)c3c(sc(C)c3C)-n3c(C)nnc32)ccc1C(=O)Nc1nc(C)c([N+](=O)[O-])s1] (S)-2-acetamido-4-((9-(2-(4-(4-chlorophenyl)-2,3,9-trimethyl-6H-thieno[3,2- f][1,2,4]triazolo[4,3-a][1,4]diazepin-6-yl)acetamido)nonyl)amino)-N-(4-methyl-5- nitrothiazol-2-yl)benzamide |
| CPD-266 | 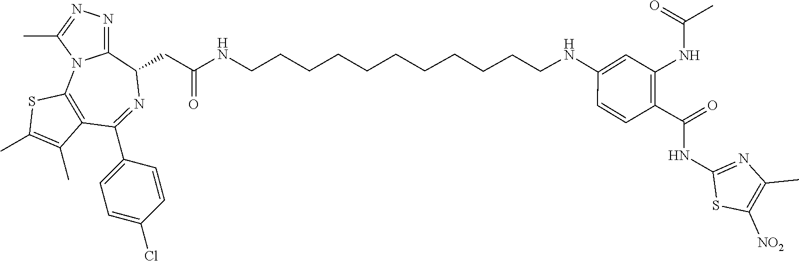 (S)-2-acetamido-4-((11-(2-(4-(4-chlorophenyl)-2,3,9-trimethyl-6H-thieno[3,2- f][1,2,4]triazolo[4,3-a][1,4]diazepin-6-yl)acetamido)undecyl)amino)-N-(4-methyl-5- nitrothiazol-2-yl)benzamide |
| CPD-267 | 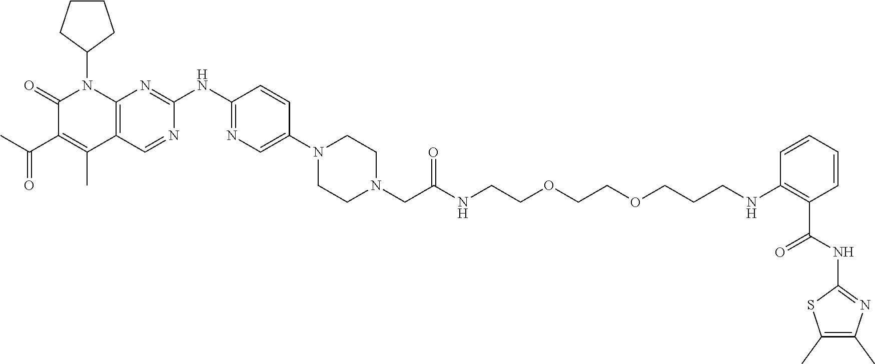 [SMILES: CC(=O)c1c(C)c2cnc(Nc3ccc(N4CCN(CC(=O)NCCOCCOCCCNc5ccccc5C(=O)Nc5nc(C)c(C)s5)CC4)cn3)nc2n(C2CCCC2)c1=O] 2-((3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propyl)amino)-N-(4,5-dimethylthiazol-2-yl)benzamide |
| CPD-268 | 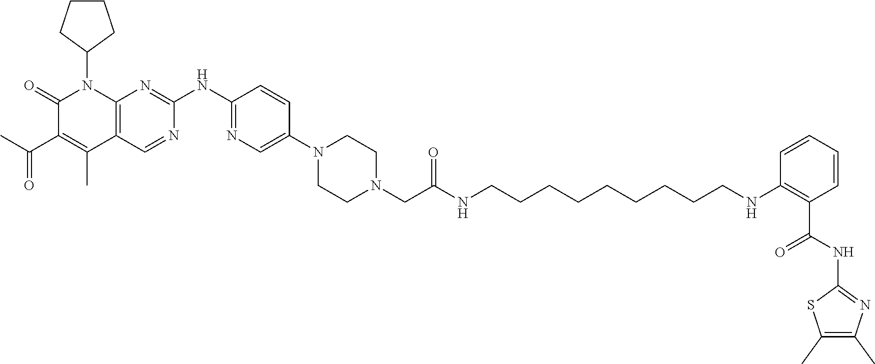 [SMILES: CC(=O)c1c(C)c2cnc(Nc3ccc(N4CCN(CC(=O)NCCCCCCCCCNc5ccccc5C(=O)Nc5nc(C)c(C)s5)CC4)cn3)nc2n(C2CCCC2)c1=O] 2-((9-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)nonyl)amino)-N-(4,5- dimethylthiazol-2-yl)benzamide |
| CPD-269 | 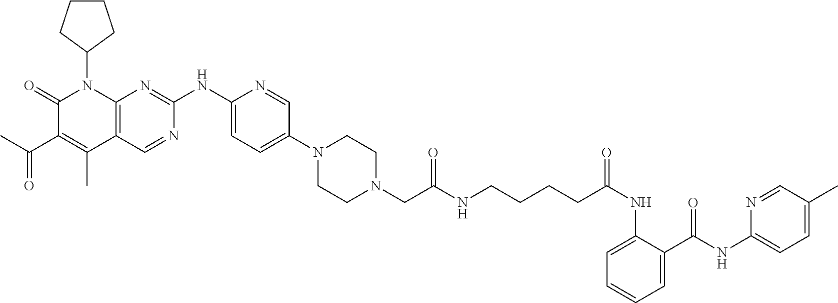 [SMILES: CC(=O)c1c(C)c2cnc(Nc3ccc(N4CCN(CC(=O)NCCCCC(=O)Nc5ccccc5C(=O)Nc5ccc(C)cn5)CC4)cn3)nc2n(C2CCCC2)c1=O] 2-(5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentanamido)-N-(5- methylpyridin-2-yl)benzamide |
| CPD-270 | 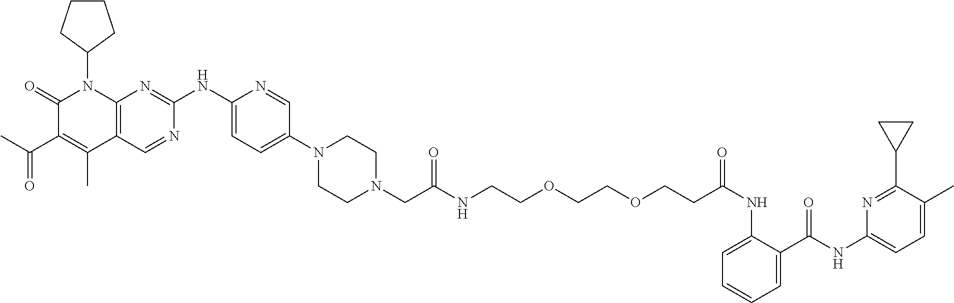 [SMILES: CC(=O)c1c(C)c2cnc(Nc3ccc(N4CCN(CC(=O)NCCOCCOCCC(=O)Nc5ccccc5C(=O)Nc5ccc(C)c(C6CC6)n5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(6-cyclopropyl-5-methylpyridin-2- yl)benzamide |
| CPD-271 | 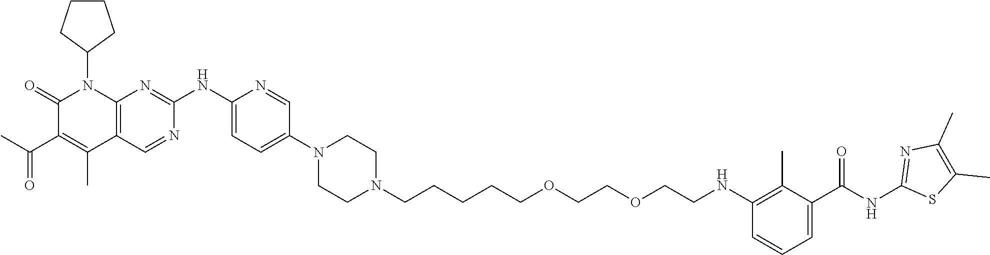 [SMILES: CC(=O)c1c(C)c2cnc(Nc3ccc(N4CCN(CCCCCOCCOCCNc5cccc(C(=O)Nc6nc(C)c(C)s6)c5C)CC4)cn3)nc2n(C2CCCC2)c1=O] 3-((2-(2-((5-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)pentyl)oxy)ethoxy)ethyl)amino)- N-(4,5-dimethylthiazol-2-yl)-2-methylbenzamide |
| CPD-272 | 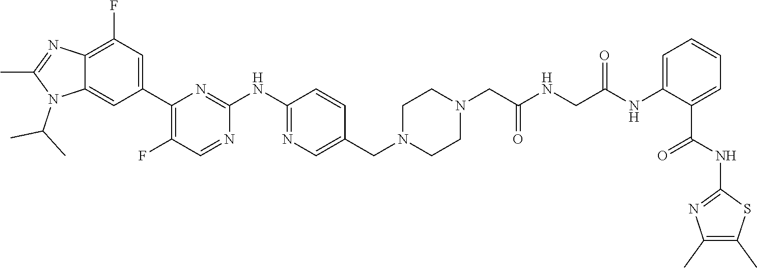 N-(4,5-dimethylthiazol-2-yl)-2-(2-(2-(4-((6-((5-fluoro-4-(4-fluoro-1-isopropyl-2- methyl-1H-benzo[d]imidazol-6-yl)pyrimidin-2-yl)amino)pyridin-3- yl)methyl)piperazin-1-yl)acetamido)acetamido)benzamide |
| CPD-273 | 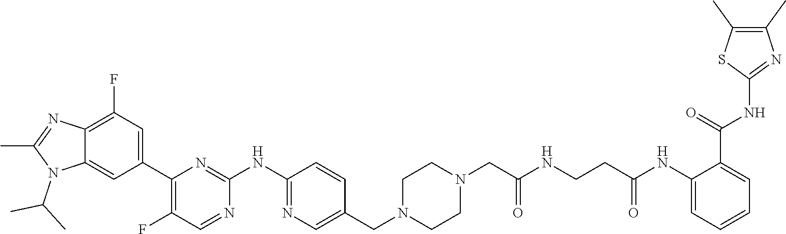 N-(4,5-dimethylthiazol-2-yl)-2-(3-(2-(4-((6-((5-fluoro-4-(4-fluoro-1-isopropyl-2- methyl-1H-benzo[d]imidazol-6-yl)pyrimidin-2-yl)amino)pyridin-3- yl)methyl)piperazin-1-yl)acetamido)propanamido)benzamide |
| CPD-274 | 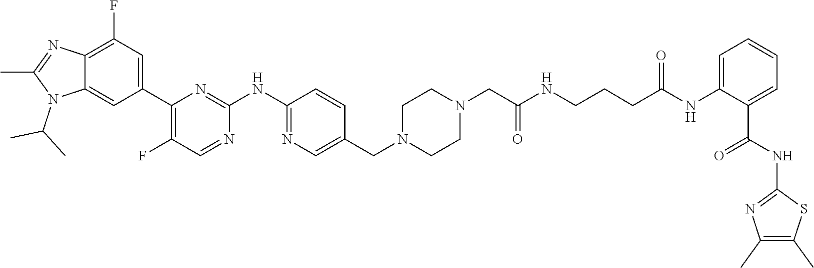 N-(4,5-dimethylthiazol-2-yl)-2-(4-(2-(4-((6-((5-fluoro-4-(4-fluoro-1-isopropyl-2- methyl-1H-benzo[d]imidazol-6-yl)pyrimidin-2-yl)amino)pyridin-3- yl)methyl)piperazin-1-yl)acetamido)butanamido)benzamide |
| CPD-275 | 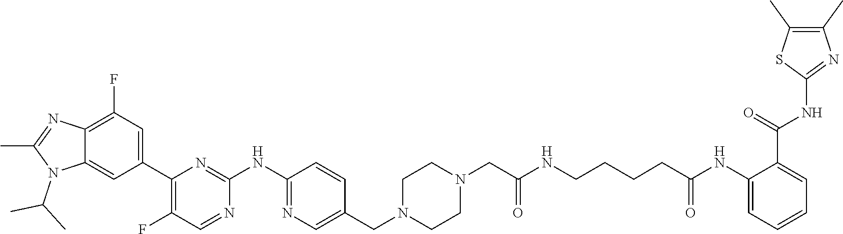 N-(4,5-dimethylthiazol-2-yl)-2-(5-(2-(4-((6-((5-fluoro-4-(4-fluoro-1-isopropyl-2- methyl-1H-benzo[d]imidazol-6-yl)pyrimidin-2-yl)amino)pyridin-3- yl)methyl)piperazin-1-yl)acetamido)pentanamido)benzamide |
| CPD-276 | 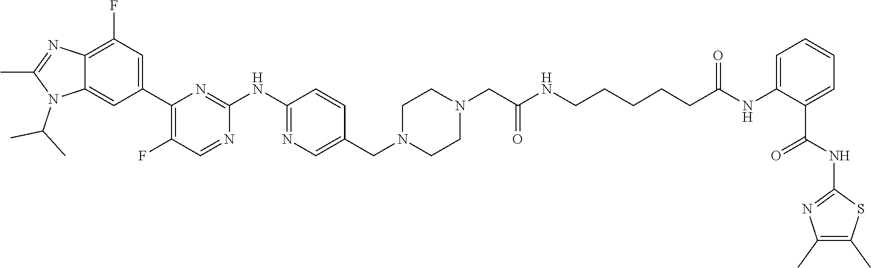 N-(4,5-dimethylthiazol-2-yl)-2-(6-(2-(4-((6-((5-fluoro-4-(4-fluoro-1-isopropyl-2- methyl-1H-benzo[d]imidazol-6-yl)pyrimidin-2-yl)amino)pyridin-3- yl)methyl)piperazin-1-yl)acetamido)hexanamido)benzamide |
| CPD-277 | 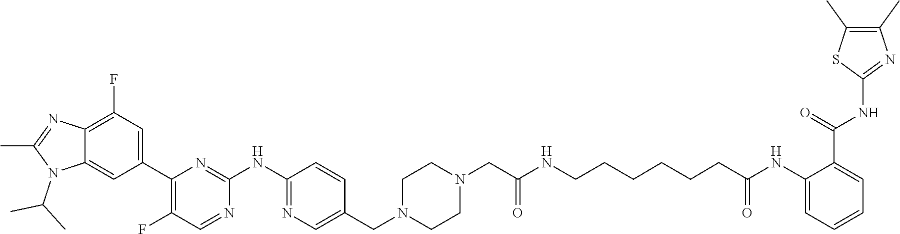 N-(4,5-dimethylthiazol-2-yl)-2-(7-(2-(4-((6-((5-fluoro-4-(4-fluoro-1-isopropyl-2- methyl-1H-benzo[d]imidazol-6-yl)pyrimidin-2-yl)amino)pyridin-3- yl)methyl)piperazin-1-yl)acetamido)heptanamido)benzamide |
| CPD-278 | 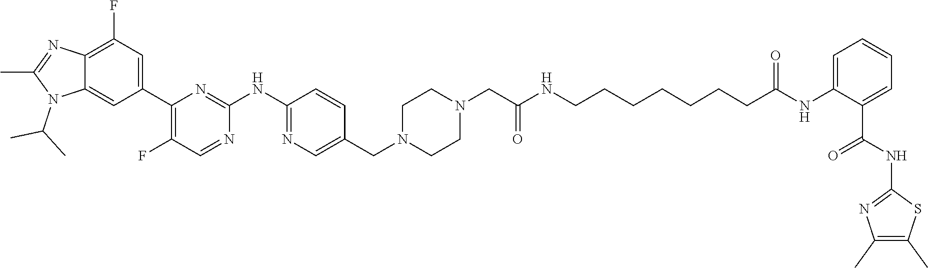 N-(4,5-dimethylthiazol-2-yl)-2-(8-(2-(4-((6-((5-fluoro-4-(4-fluoro-1-isopropyl-2- methyl-1H-benzo[d]imidazol-6-yl)pyrimidin-2-yl)amino)pyridin-3- yl)methyl)piperazin-1-yl)acetamido)octanamido)benzamide |
| CPD-279 | 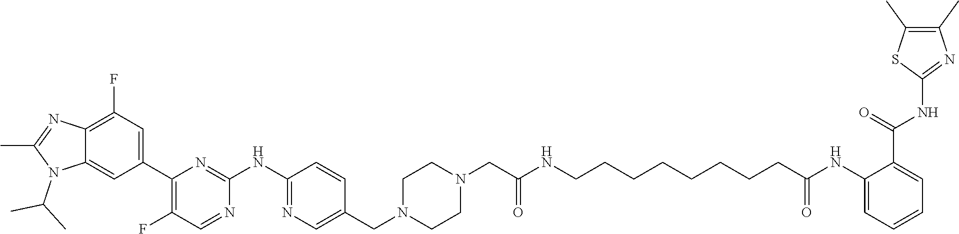 N-(4,5-dimethylthiazol-2-yl)-2-(9-(2-(4-((6-((5-fluoro-4-(4-fluoro-1-isopropyl-2- methyl-1H-benzo[d]imidazol-6-yl)pyrimidin-2-yl)amino)pyridin-3- yl)methyl)piperazin-1-yl)acetamido)nonanamido)benzamide |
| CPD-280 | 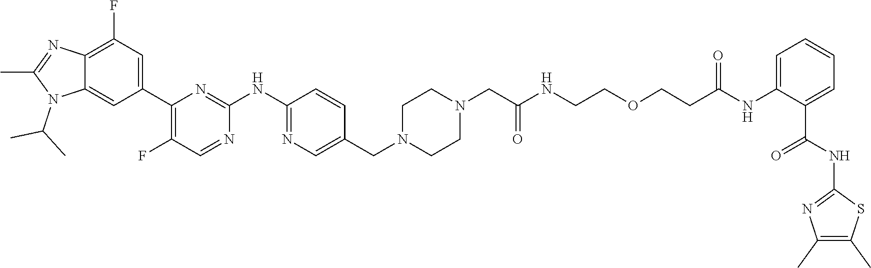 N-(4,5-dimethylthiazol-2-yl)-2-(3-(2-(2-(4-((6-((5-fluoro-4-(4-fluoro-1-isopropyl-2- methyl-1H-benzo[d]imidazol-6-yl)pyrimidin-2-yl)amino)pyridin-3- yl)methyl)piperazin-1-yl)acetamido)ethoxy)propanamido)benzamide |
| CPD-281 | 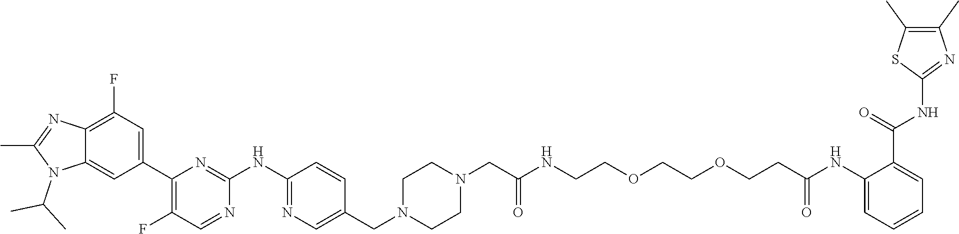 N-(4,5-dimethylthiazol-2-yl)-2-(3-(2-(2-(2-(4-((6-((5-fluoro-4-(4-fluoro-1-isopropyl- 2-methyl-1H-benzo[d]imidazol-6-yl)pyrimidin-2-yl)amino)pyridin-3- yl)methyl)piperazin-1-yl)acetamido)ethoxy)ethoxy)propanamido)benzamide |
| CPD-282 | 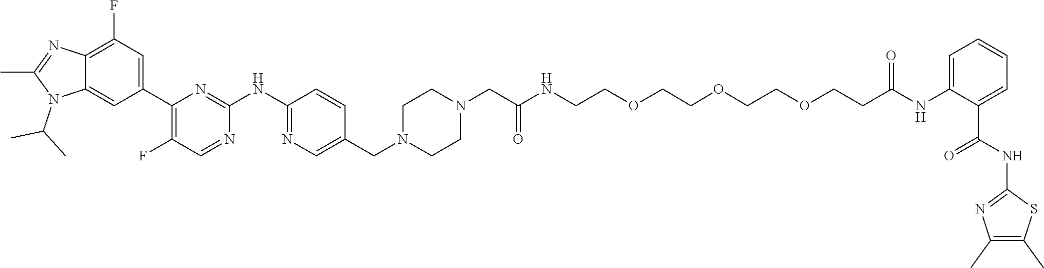 N-(4,5-dimethylthiazol-2-yl)-2-(1-(4-((6-((5-fluoro-4-(4-fluoro-1-isopropyl-2-methyl- 1H-benzo[d]imidazol-6-yl)pyrimidin-2-yl)amino)pyridin-3-yl)methyl)piperazin-1-yl)- 2-oxo-6,9,12-trioxa-3-azapentadecan-15-amido)benzamide |
| CPD-283 | 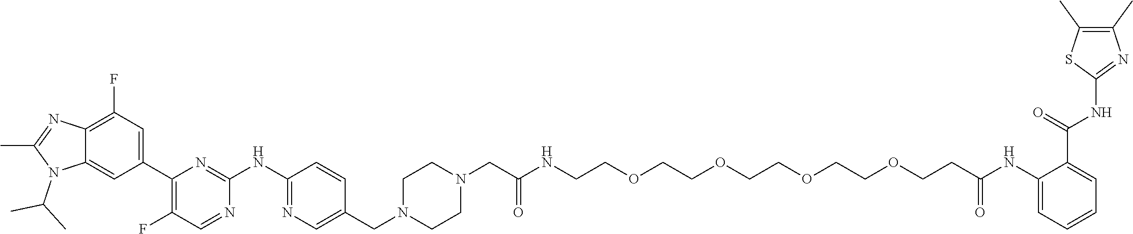 N-(4,5-dimethylthiazol-2-yl)-2-(1-(4-((6-((5-fluoro-4-(4-fluoro-1-isopropyl-2-methyl- 1H-benzo[d]imidazol-6-yl)pyrimidin-2-yl)amino)pyridin-3-yl)methyl)piperazin-1-yl)- 2-oxo-6,9,12,15-tetraoxa-3-azaoctadecan-18-amido)benzamide |
| CPD-284 | 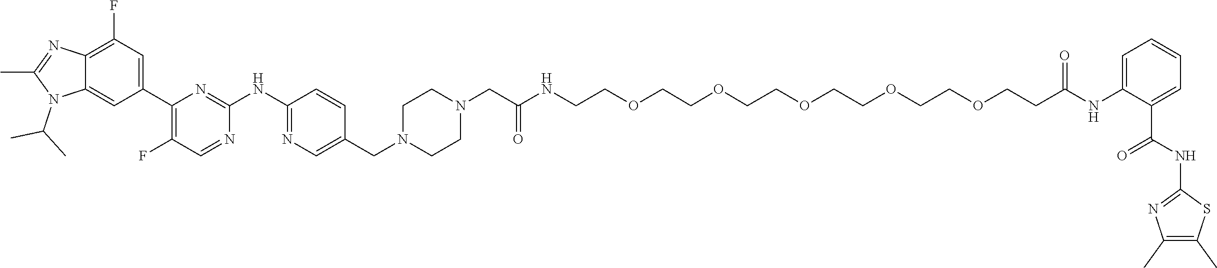 N-(4,5-dimethylthiazol-2-yl)-2-(1-(4-((6-((5-fluoro-4-(4-fluoro-1-isopropyl-2-methyl- 1H-benzo[d]imidazol-6-yl)pyrimidin-2-yl)amino)pyridin-3-yl)methyl)piperazin-1-yl)- 2-oxo-6,9,12,15,18-pentaoxa-3-azahenicosan-21-amido)benzamide |
| CPD-285 | 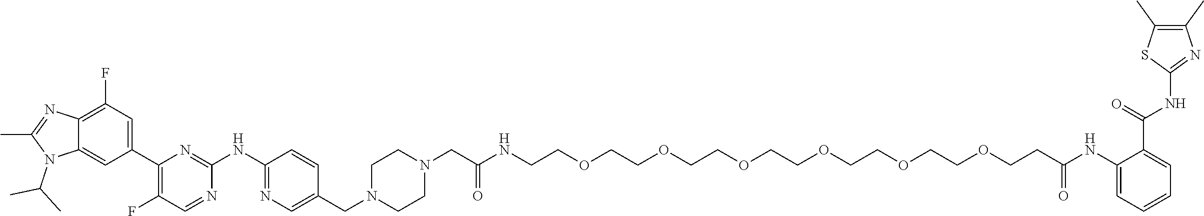 N-(4,5-dimethylthiazol-2-yl)-2-(1-(4-((6-((5-fluoro-4-(4-fluoro-1-isopropyl-2-methyl- 1H-benzo[d]imidazol-6-yl)pyrimidin-2-yl)amino)pyridin-3-yl)methyl)piperazin-1-yl)- 2-oxo-6,9,12,15,18,21-hexaoxa-3-azatetracosan-24-amido)benzamide |
| CPD-286 | 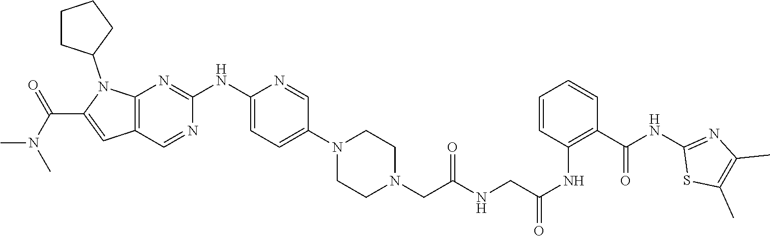 7-cyclopentyl-2-((5-(4-(2-((2-((2-((4,5-dimethylthiazol-2- yl)carbamoyl)phenyl)amino)-2-oxoethyl)amino)-2-oxoethyl)piperazin-1-yl)pyridin-2- yl)amino)-N,N-dimethyl-7H-pyrrolo[2,3-d]pyrimidine-6-carboxamide |
| CPD-287 | 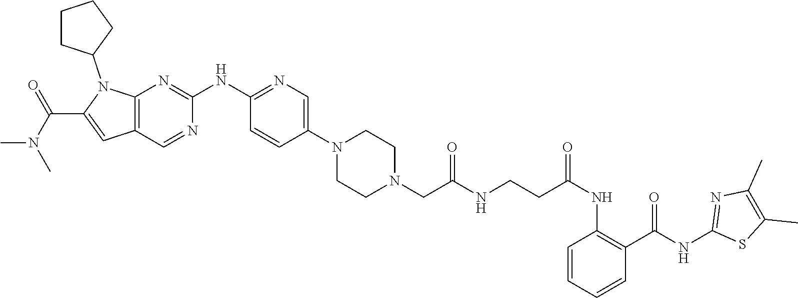 7-cyclopentyl-2-((5-(4-(2-((3-((2-((4,5-dimethylthiazol-2- yl)carbamoyl)phenyl)amino)-3-oxopropyl)amino)-2-oxoethyl)piperazin-1-yl)pyridin- 2-yl)amino)-N,N-dimethyl-7H-pyrrolo[2,3-d]pyrimidine-6-carboxamide |
| CPD-288 | 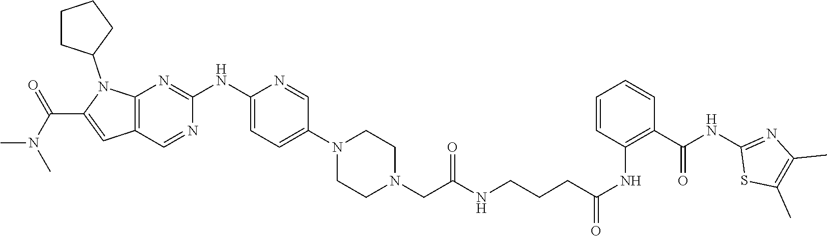 7-cyclopentyl-2-((5-(4-(2-((4-((2-((4,5-dimethylthiazol-2- yl)carbamoyl)phenyl)amino)-4-oxobutyl)amino)-2-oxoethyl)piperazin-1-yl)pyridin-2- yl)amino)-N,N-dimethyl-7H-pyrrolo[2,3-d]pyrimidine-6-carboxamide |
| CPD-289 | 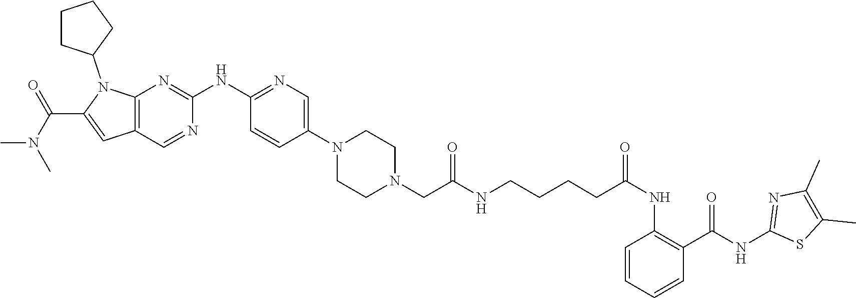 7-cyclopentyl-2-((5-(4-(2-((5-((2-((4,5-dimethylthiazol-2- yl)carbamoyl)phenyl)amino)-5-oxopentyl)amino)-2-oxoethyl)piperazin-1-yl)pyridin- 2-yl)amino)-N,N-dimethyl-7H-pyrrolo[2,3-d]pyrimidine-6-carboxamide |
| CPD-290 | 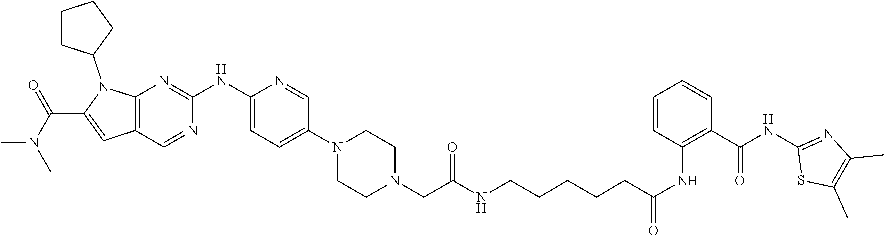 7-cyclopentyl-2-((5-(4-(2-((6-((2-((4,5-dimethylthiazol-2- yl)carbamoyl)phenyl)amino)-6-oxohexyl)amino)-2-oxoethyl)piperazin-1-yl)pyridin-2- yl)amino)-N,N-dimethyl-7H-pyrrolo[2,3-d]pyrimidine-6-carboxamide |
| CPD-291 | 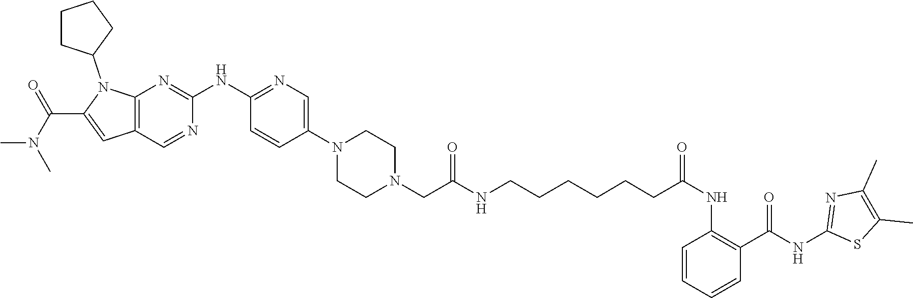 7-cyclopentyl-2-((5-(4-(2-((7-((2-((4,5-dimethylthiazol-2- yl)carbamoyl)phenyl)amino)-7-oxoheptyl)amino)-2-oxoethyl)piperazin-1-yl)pyridin- 2-yl)amino)-N,N-dimethyl-7H-pyrrolo[2,3-d]pyrimidine-6-carboxamide |
| CPD-292 | 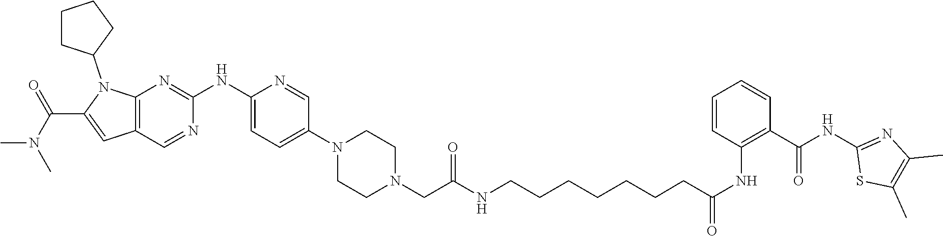 7-cyclopentyl-2-((5-(4-(2-((8-((2-((4,5-dimethylthiazol-2- yl)carbamoyl)phenyl)amino)-8-oxooctyl)amino)-2-oxoethyl)piperazin-1-yl)pyridin-2- yl)amino)-N,N-dimethyl-7H-pyrrolo[2,3-d]pyrimidine-6-carboxamide |
| CPD-293 | 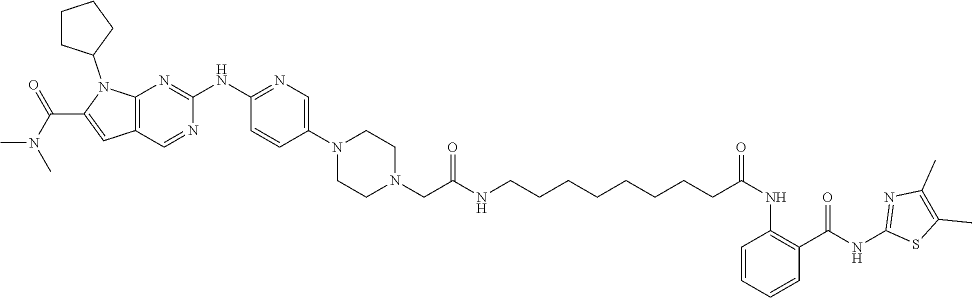 7-cyclopentyl-2-((5-(4-(2-((9-((2-((4,5-dimethylthiazol-2- yl)carbamoyl)phenyl)amino)-9-oxononyl)amino)-2-oxoethyl)piperazin-1-yl)pyridin- 2-yl)amino)-N,N-dimethyl-7H-pyrrolo[2,3-d]pyrimidine-6-carboxamide |
| CPD-294 | 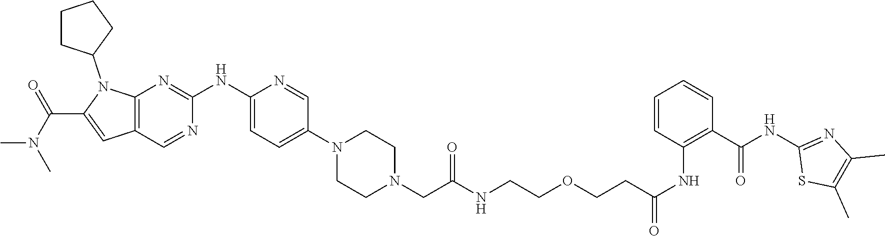 7-cyclopentyl-2-((5-(4-(2-((2-(3-((2-((4,5-dimethylthiazol-2- yl)carbamoyl)phenyl)amino)-3-oxopropoxy)ethyl)amino)-2-oxoethyl)piperazin-1- yl)pyridin-2-yl)amino)-N,N-dimethyl-7H-pyrrolo[2,3-d]pyrimidine-6-carboxamide |
| CPD-295 | 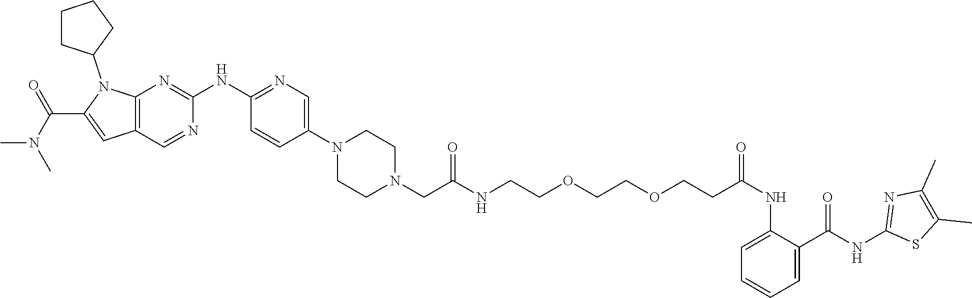 7-cyclopentyl-2-((5-(4-(2-((2-(2-(3-((2-((4,5-dimethylthiazol-2- yl)carbamoyl)phenyl)amino)-3-oxopropoxy)ethoxy)ethyl)amino)-2- oxoethyl)piperazin-1-yl)pyridin-2-yl)amino)-N,N-dimethyl-7H-pyrrolo[2,3- d]pyrimidine-6-carboxamide |
| CPD-296 | 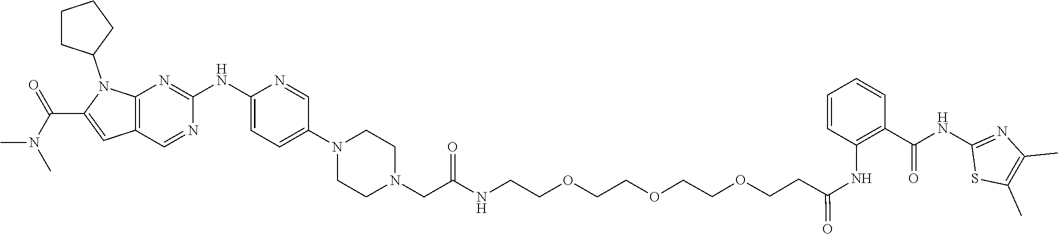 7-cyclopentyl-2-((5-(4-(15-((2-((4,5-dimethylthiazol-2-yl)carbamoyl)phenyl)amino)- 2,15-dioxo-6,9,12-trioxa-3-azapentadecyl)piperazin-1-yl)pyridin-2-yl)amino)-N,N- dimethyl-7H-pyrrolo[2,3-d]pyrimidine-6-carboxamide |
| CPD-297 | 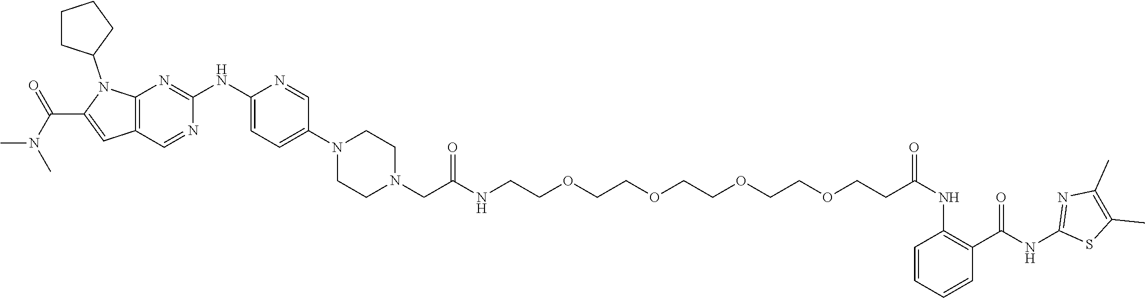 7-cyclopentyl-2-((5-(4-(18-((2-((4,5-dimethylthiazol-2-yl)carbamoyl)phenyl)amino)- 2,18-dioxo-6,9,12,15-tetraoxa-3-azaoctadecyl)piperazin-1-yl)pyridin-2-yl)amino)- N,N-dimethyl-7H-pyrrolo[2,3-d]pyrimidine-6-carboxamide |
| CPD-298 | 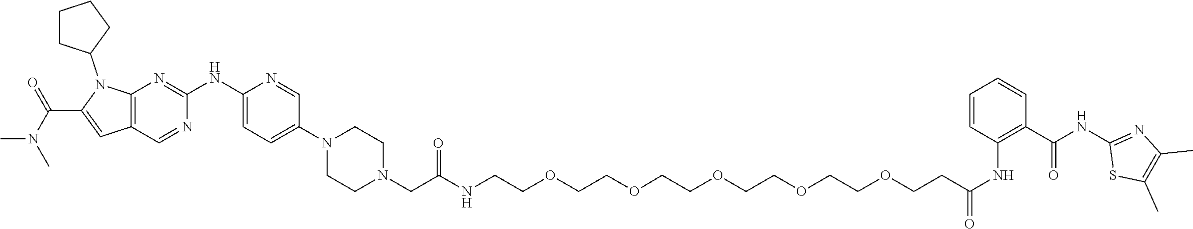 7-cyclopentyl-2-((5-(4-(21-((2-((4,5-dimethylthiazol-2-yl)carbamoyl)phenyl)amino)- 2,21-dioxo-6,9,12,15,18-pentaoxa-3-azahenicosyl)piperazin-1-yl)pyridin-2-yl)amino)- N,N-dimethyl-7H-pyrrolo[2,3-d]pyrimidine-6-carboxamide |
| CPD-299 | 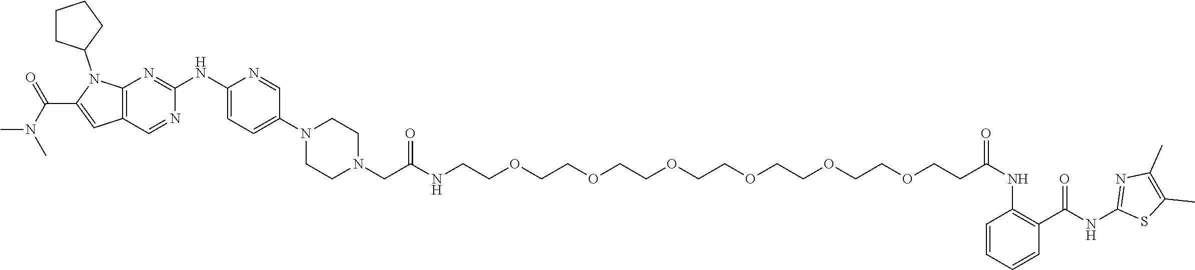 7-cyclopentyl-2-((5-(4-(24-((2-((4,5-dimethylthiazol-2-yl)carbamoyl)phenyl)amino)- 2,24-dioxo-6,9,12,15,18,21-hexaoxa-3-azatetracosyl)piperazin-1-yl)pyridin-2- yl)amino)-N,N-dimethyl-7H-pyrrolo[2,3-d]pyrimidine-6-carboxamide |
| CPD-300 | 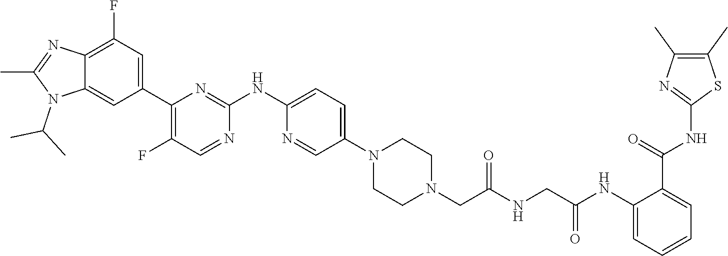 N-(4,5-dimethylthiazol-2-yl)-2-(2-(2-(4-(6-((5-fluoro-4-(4-fluoro-1-isopropyl-2- methyl-1H-benzo[d]imidazol-6-yl)pyrimidin-2-yl)amino)pyridin-3-yl)piperazin-1- yl)acetamido)acetamido)benzamide |
| CPD-301 | 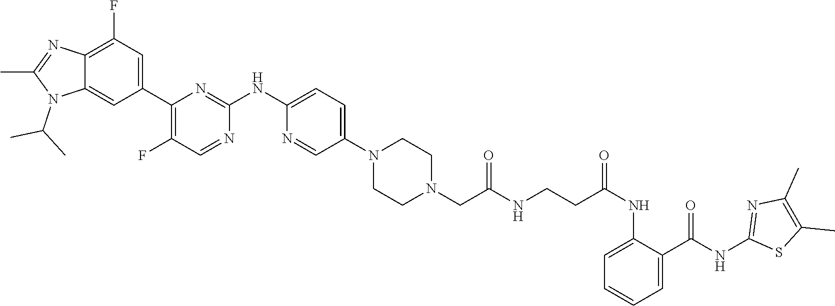 N-(4,5-dimethylthiazol-2-yl)-2-(3-(2-(4-(6-((5-fluoro-4-(4-fluoro-1-isopropyl-2- methyl-1H-benzo[d]imidazol-6-yl)pyrimidin-2-yl)amino)pyridin-3-yl)piperazin-1- yl)acetamido)propanamido)benzamide |
| CPD-302 | 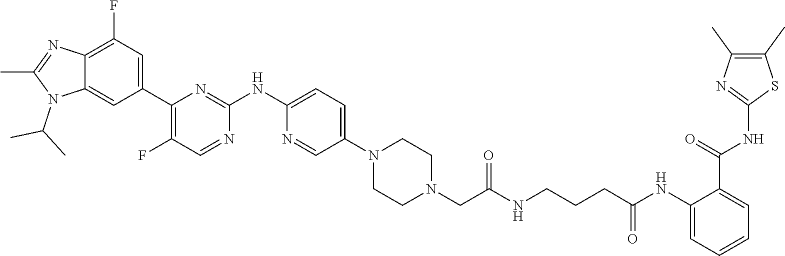 N-(4,5-dimethylthiazol-2-yl)-2-(4-(2-(4-(6-((5-fluoro-4-(4-fluoro-1-isopropyl-2- methyl-1H-benzo[d]imidazol-6-yl)pyrimidin-2-yl)amino)pyridin-3-yl)piperazin-1- yl)acetamido)butanamido)benzamide |
| CPD-303 | 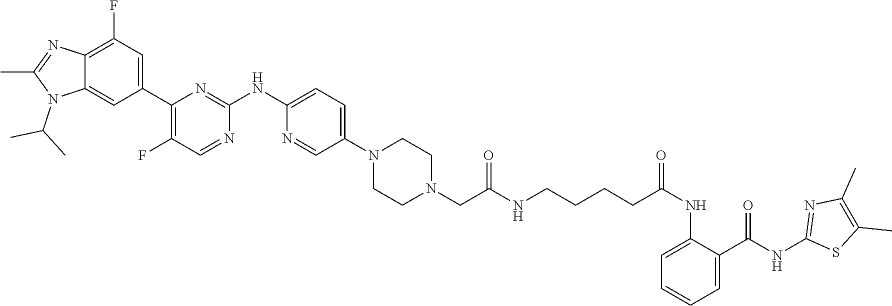 N-(4,5-dimethylthiazol-2-yl)-2-(5-(2-(4-(6-((5-fluoro-4-(4-fluoro-1-isopropyl-2- methyl-1H-benzo[d]imidazol-6-yl)pyrimidin-2-yl)amino)pyridin-3-yl)piperazin-1- yl)acetamido)pentanamido)benzamide |
| CPD-304 | 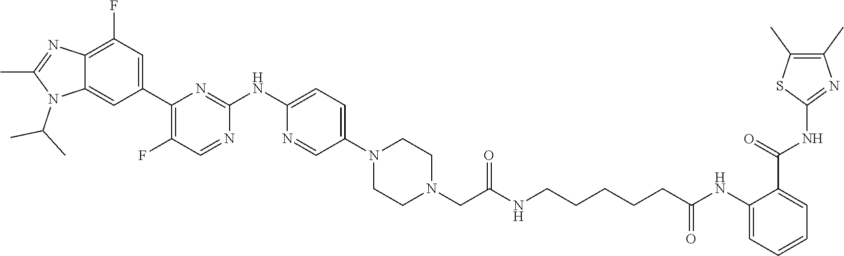 N-(4,5-dimethylthiazol-2-yl)-2-(6-(2-(4-(6-((5-fluoro-4-(4-fluoro-1-isopropyl-2- methyl-1H-benzo[d]imidazol-6-yl)pyrimidin-2-yl)amino)pyridin-3-yl)piperazin-1- yl)acetamido)hexanamido)benzamide |
| CPD-305 | 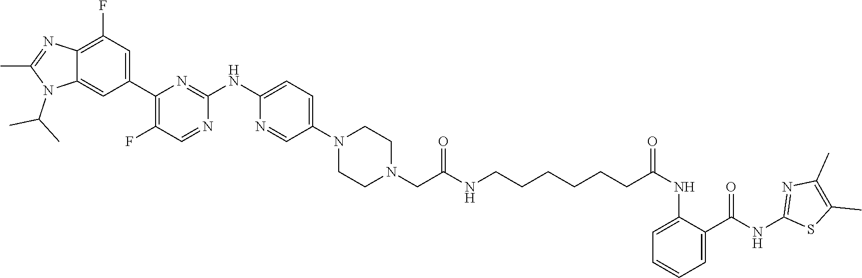 N-(4,5-dimethylthiazol-2-yl)-2-(7-(2-(4-(6-((5-fluoro-4-(4-fluoro-1-isopropyl-2- methyl-1H-benzo[d]imidazol-6-yl)pyrimidin-2-yl)amino)pyridin-3-yl)piperazin-1- yl)acetamido)heptanamido)benzamide |
| CPD-306 | 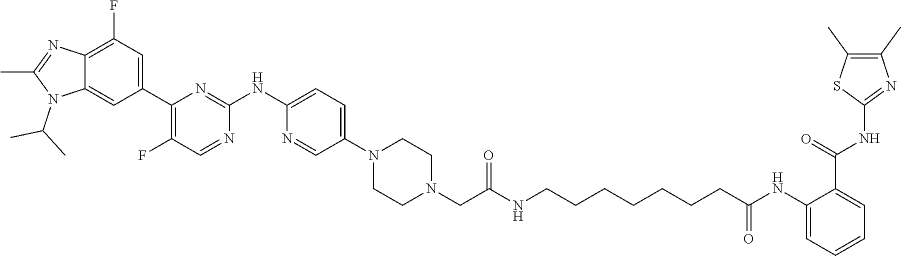 N-(4,5-dimethylthiazol-2-yl)-2-(8-(2-(4-(6-((5-fluoro-4-(4-fluoro-1-isopropyl-2- methyl-1H-benzo[d]imidazol-6-yl)pyrimidin-2-yl)amino)pyridin-3-yl)piperazin-1- yl)acetamido)octanamido)benzamide |
| CPD-307 | 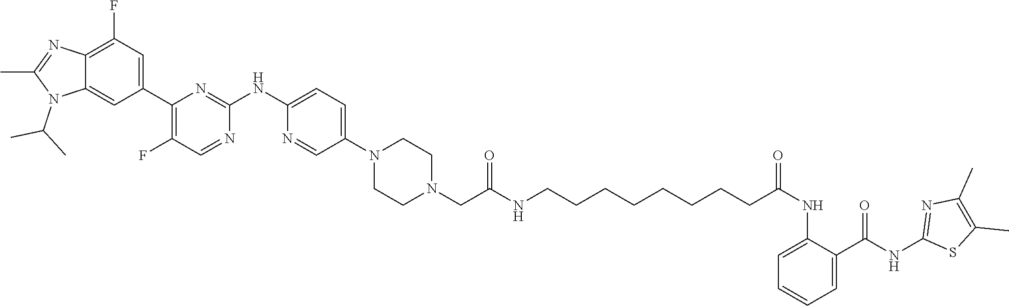 N-(4,5-dimethylthiazol-2-yl)-2-(9-(2-(4-(6-((5-fluoro-4-(4-fluoro-1-isopropyl-2- methyl-1H-benzo[d]imidazol-6-yl)pyrimidin-2-yl)amino)pyridin-3-yl)piperazin-1- yl)acetamido)nonanamido)benzamide |
| CPD-308 | 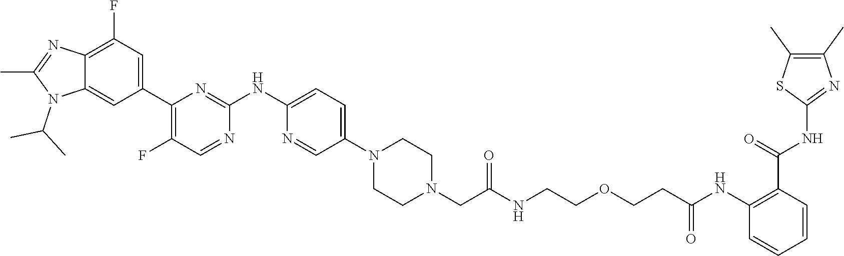 N-(4,5-dimethylthiazol-2-yl)-2-(3-(2-(2-(4-(6-((5-fluoro-4-(4-fluoro-1-isopropyl-2- methyl-1H-benzo[d]imidazol-6-yl)pyrimidin-2-yl)amino)pyridin-3-yl)piperazin-1- yl)acetamido)ethoxy)propanamido)benzamide |
| CPD-309 | 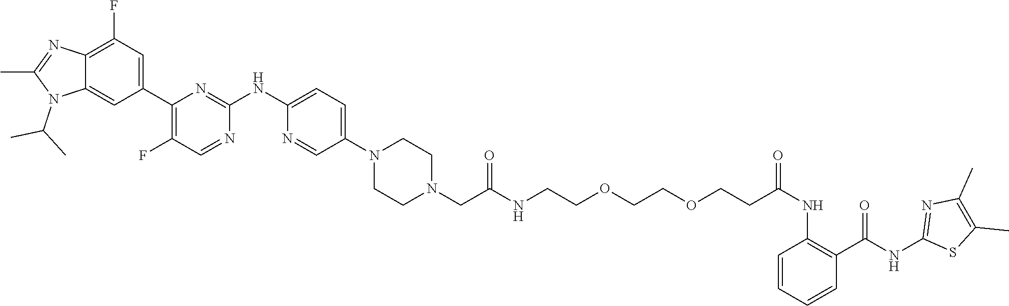 N-(4,5-dimethylthiazol-2-yl)-2-(3-(2-(2-(2-(4-(6-((5-fluoro-4-(4-fluoro-1-isopropyl-2- methyl-1H-benzo[d]imidazol-6-yl)pyrimidin-2-yl)amino)pyridin-3-yl)piperazin-1- yl)acetamido)ethoxy)ethoxy)propanamido)benzamide |
| CPD-310 | 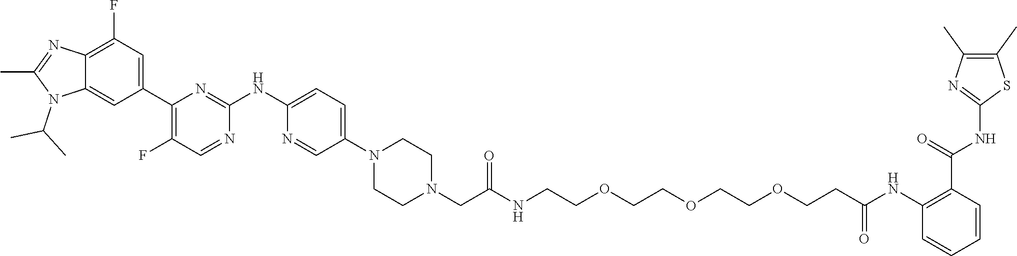 N-(4,5-dimethylthiazol-2-yl)-2-(1-(4-(6-((5-fluoro-4-(4-fluoro-1-isopropyl-2-methyl- 1H-benzo[d]imidazol-6-yl)pyrimidin-2-yl)amino)pyridin-3-yl)piperazin-1-yl)-2-oxo- 6,9,12-trioxa-3-azapentadecan-15-amido)benzamide |
| CPD-311 | 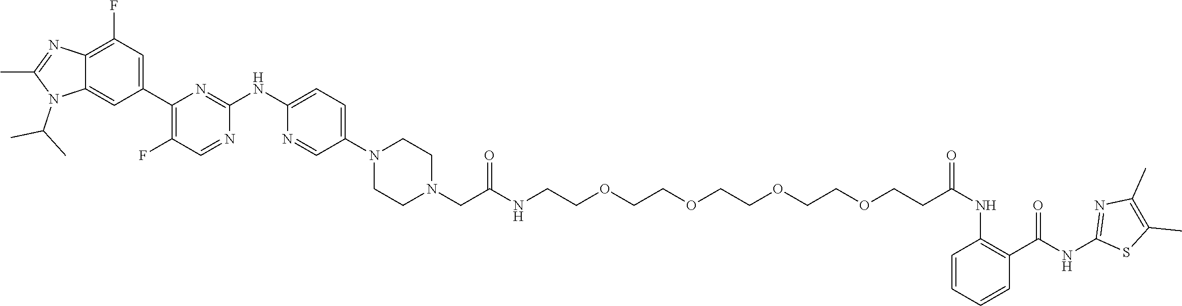 N-(4,5-dimethylthiazol-2-yl)-2-(1-(4-(6-((5-fluoro-4-(4-fluoro-1-isopropyl-2-methyl- 1H-benzo[d]imidazol-6-yl)pyrimidin-2-yl)amino)pyridin-3-yl)piperazin-1-yl)-2-oxo- 6,9,12,15-tetraoxa-3-azaoctadecan-18-amido)benzamide |
| CPD-312 | 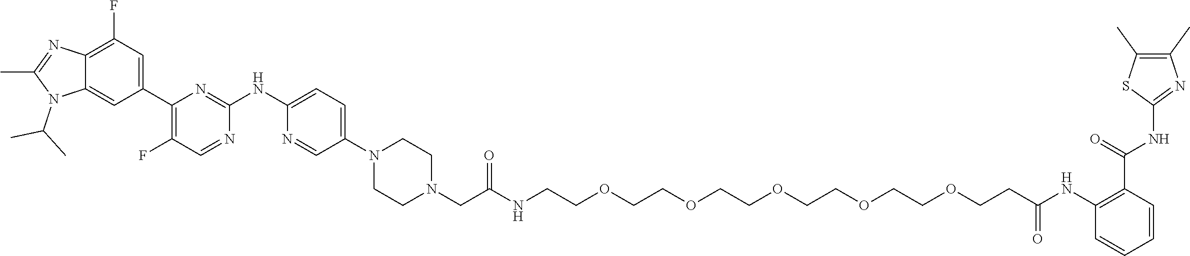 N-(4,5-dimethylthiazol-2-yl)-2-(1-(4-(6-((5-fluoro-4-(4-fluoro-1-isopropyl-2-methyl- 1H-benzo[d]imidazol-6-yl)pyrimidin-2-yl)amino)pyridin-3-yl)piperazin-1-yl)-2-oxo- 6,9,12,15,18-pentaoxa-3-azahenicosan-21-amido)benzamide |
| CPD-313 | 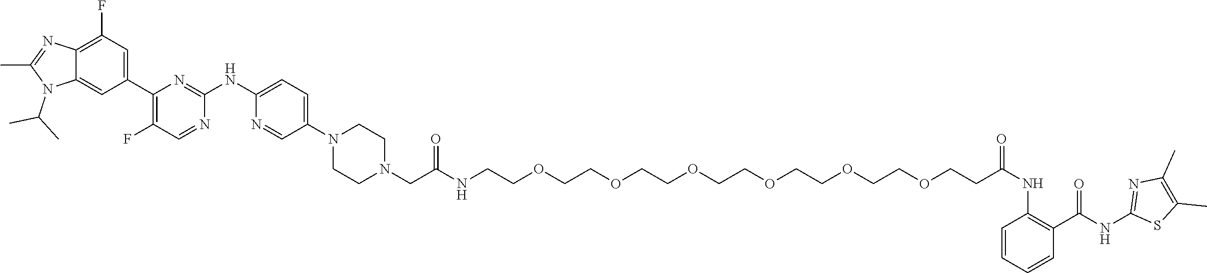 N-(4,5-dimethylthiazol-2-yl)-2-(1-(4-(6-((5-fluoro-4-(4-fluoro-1-isopropyl-2-methyl- 1H-benzo[d]imidazol-6-yl)pyrimidin-2-yl)amino)pyridin-3-yl)piperazin-1-yl)-2-oxo- 6,9,12,15,18,21-hexaoxa-3-azatetracosan-24-amido)benzamide |
| CPD-314 | 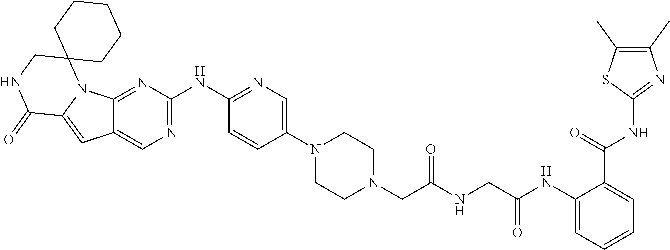 [SMILES: Cc1nc(NC(=O)c2ccccc2NC(=O)CNC(=O)CN2CCN(c3ccc(Nc4ncc5cc6n(c5n4)C4(CCCCC4)CNC6=O)nc3)CC2)sc1C] N-(4,5-dimethylthiazol-2-yl)-2-(2-(2-(4-(6-((6′-oxo-7′,8′-dihydro-6′H- spiro[cyclohexane-1,9′-pyrazino[1′,2′:1,5]pyrrolo[2,3-d]pyrimidin]-2′- yl)amino)pyridin-3-yl)piperazin-1-yl)acetamido)acetamido)benzamide |
| CPD-315 | 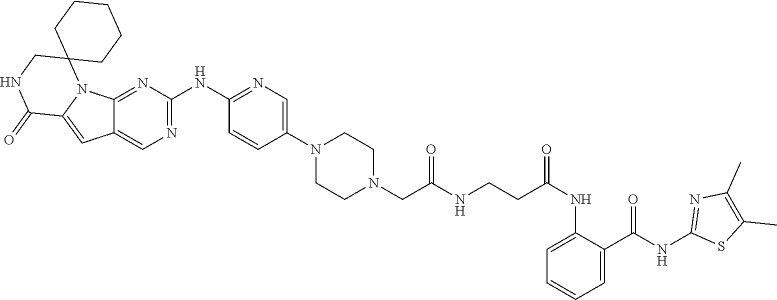 [SMILES: Cc1nc(NC(=O)c2ccccc2NC(=O)CCNC(=O)CN2CCN(c3ccc(Nc4ncc5cc6n(c5n4)C4(CCCCC4)CNC6=O)nc3)CC2)sc1C] N-(4,5-dimethylthiazol-2-yl)-2-(3-(2-(4-(6-((6′-oxo-7′,8′-dihydro-6′H- spiro[cyclohexane-1,9′-pyrazino[1′,2′:1,5]pyrrolo[2,3-d]pyrimidin]-2′- yl)amino)pyridin-3-yl)piperazin-1-yl)acetamido)propanamido)benzamide |
| CPD-316 | 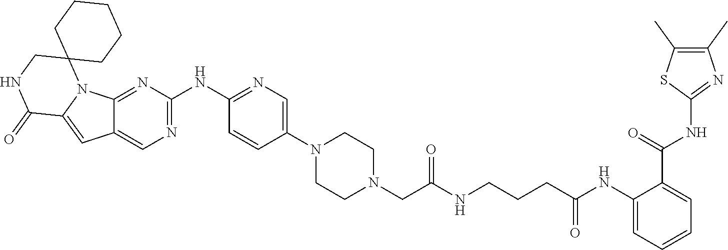 [SMILES: Cc1nc(NC(=O)c2ccccc2NC(=O)CCCNC(=O)CN2CCN(c3ccc(Nc4ncc5cc6n(c5n4)C4(CCCCC4)CNC6=O)nc3)CC2)sc1C] N-(4,5-dimethylthiazol-2-yl)-2-(4-(2-(4-(6-((6′-oxo-7′,8′-dihydro-6′H- spiro[cyclohexane-1,9′-pyrazino[1′,2′:1,5]pyrrolo[2,3-d]pyrimidin]-2′- yl)amino)pyridin-3-yl)piperazin-1-yl)acetamido)butanamido)benzamide |
| CPD-317 | 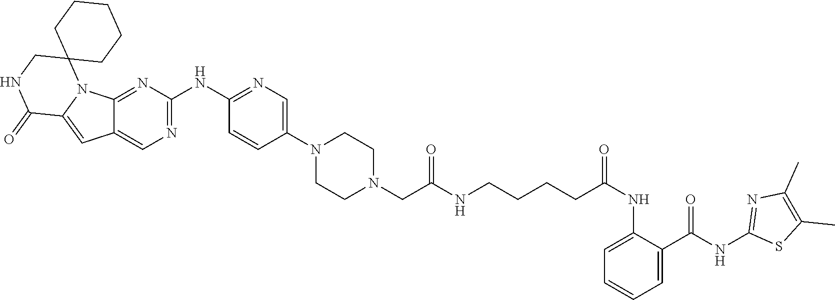 [SMILES: Cc1nc(NC(=O)c2ccccc2NC(=O)CCCCNC(=O)CN2CCN(c3ccc(Nc4ncc5cc6n(c5n4)C4(CCCCC4)CNC6=O)nc3)CC2)sc1C] N-(4,5-dimethylthiazol-2-yl)-2-(5-(2-(4-(6-((6′-oxo-7′,8′-dihydro-6′H- spiro[cyclohexane-1,9′-pyrazino[1′,2′:1,5]pyrrolo[2,3-d]pyrimidin]-2′- yl)amino)pyridin-3-yl)piperazin-1-yl)acetamido)pentanamido)benzamide |
| CPD-318 | 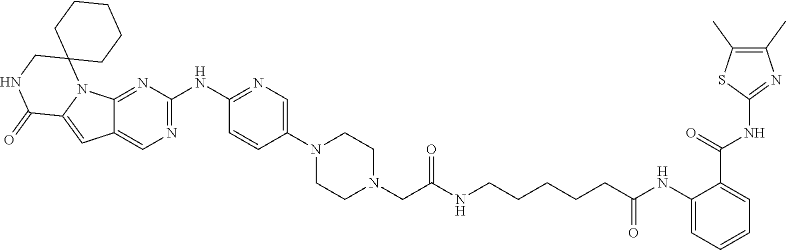 [SMILES: Cc1nc(NC(=O)c2ccccc2NC(=O)CCCCCNC(=O)CN2CCN(c3ccc(Nc4ncc5cc6n(c5n4)C4(CCCCC4)CNC6=O)nc3)CC2)sc1C] N-(4,5-dimethylthiazol-2-yl)-2-(6-(2-(4-(6-((6′-oxo-7′,8′-dihydro-6′H- spiro[cyclohexane-1,9′-pyrazino[1′,2′:1,5]pyrrolo[2,3-d]pyrimidin]-2′- yl)amino)pyridin-3-yl)piperazin-1-yl)acetamido)hexanamido)benzamide |
| CPD-319 | 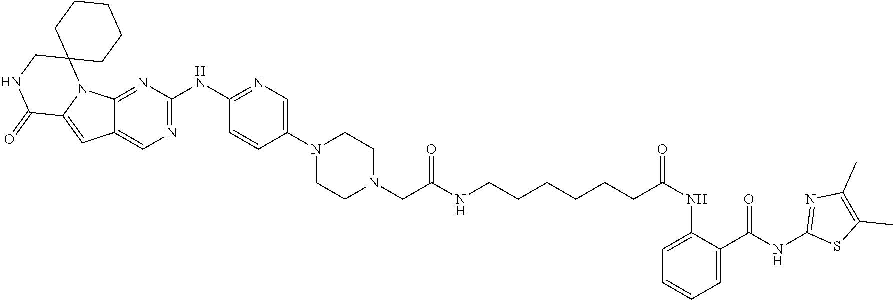 [SMILES: Cc1nc(NC(=O)c2ccccc2NC(=O)CCCCCCNC(=O)CN2CCN(c3ccc(Nc4ncc5cc6n(c5n4)C4(CCCCC4)CNC6=O)nc3)CC2)sc1C] N-(4,5-dimethylthiazol-2-yl)-2-(7-(2-(4-(6-((6′-oxo-7′,8′-dihydro-6′H- spiro[cyclohexane-1,9′-pyrazino[1′,2′:1,5]pyrrolo[2,3-d]pyrimidin]-2′- yl)amino)pyridin-3-yl)piperazin-1-yl)acetamido)heptanamido)benzamide |
| CPD-320 | 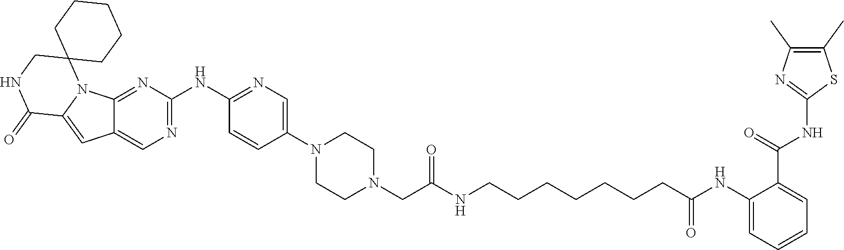 [SMILES: Cc1nc(NC(=O)c2ccccc2NC(=O)CCCCCCCNC(=O)CN2CCN(c3ccc(Nc4ncc5cc6n(c5n4)C4(CCCCC4)CNC6=O)nc3)CC2)sc1C] N-(4,5-dimethylthiazol-2-yl)-2-(8-(2-(4-(6-((6′-oxo-7′,8′-dihydro-6′H- spiro[cyclohexane-1,9′-pyrazino[1′,2′:1,5]pyrrolo[2,3-d]pyrimidin]-2′- yl)amino)pyridin-3-yl)piperazin-1-yl)acetamido)octanamido)benzamide |
| CPD-321 | 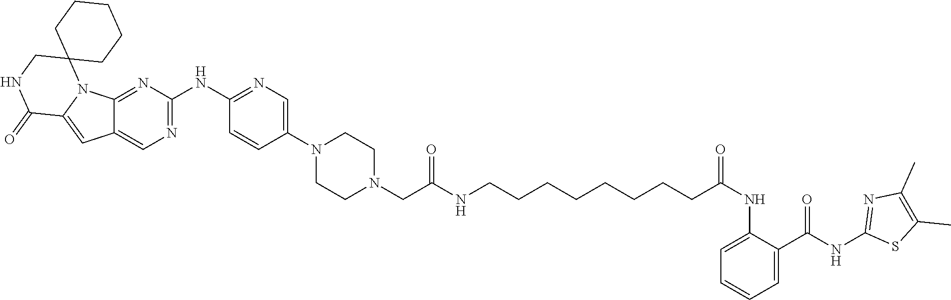 [SMILES: Cc1nc(NC(=O)c2ccccc2NC(=O)CCCCCCCCNC(=O)CN2CCN(c3ccc(Nc4ncc5cc6n(c5n4)C4(CCCCC4)CNC6=O)nc3)CC2)sc1C] N-(4,5-dimethylthiazol-2-yl)-2-(9-(2-(4-(6-((6′-oxo-7′,8′-dihydro-6′H- spiro[cyclohexane-1,9′-pyrazino[1′,2′:1,5]pyrrolo[2,3-d]pyrimidin]-2′- yl)amino)pyridin-3-yl)piperazin-1-yl)acetamido)nonanamido)benzamide |
| CPD-322 | 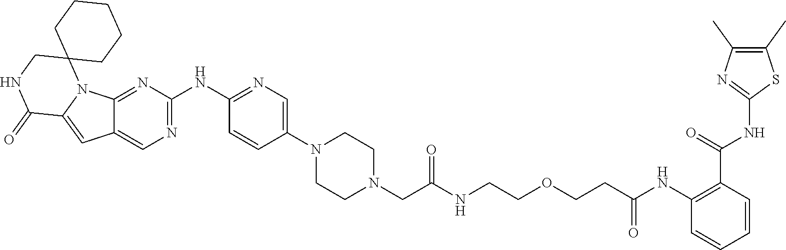 [SMILES: Cc1nc(NC(=O)c2ccccc2NC(=O)CCOCCNC(=O)CN2CCN(c3ccc(Nc4ncc5cc6n(c5n4)C4(CCCCC4)CNC6=O)nc3)CC2)sc1C] N-(4,5-dimethylthiazol-2-yl)-2-(3-(2-(2-(4-(6-((6′-oxo-7′,8′-dihydro-6′H- spiro[cyclohexane-1,9′-pyrazino[1′,2′:1,5]pyrrolo[2,3-d]pyrimidin]-2′- yl)amino)pyridin-3-yl)piperazin-1-yl)acetamido)ethoxy)propanamido)benzamide |
| CPD-323 | 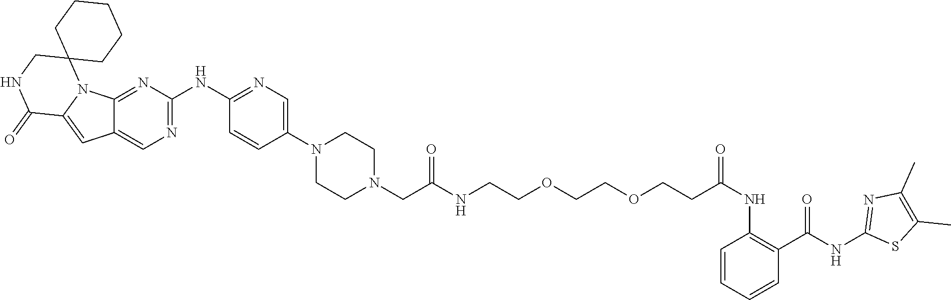 [SMILES: Cc1nc(NC(=O)c2ccccc2NC(=O)CCOCCOCCNC(=O)CN2CCN(c3ccc(Nc4ncc5cc6n(c5n4)C4(CCCCC4)CNC6=O)nc3)CC2)sc1C] N-(4,5-dimethylthiazol-2-yl)-2-(3-(2-(2-(2-(4-(6-((6′-oxo-7′,8′-dihydro-6′H- spiro[cyclohexane-1,9′-pyrazino[1′,2′:1,5]pyrrolo[2,3-d]pyrimidin]-2′- yl)amino)pyridin-3-yl)piperazin-1- yl)acetamido)ethoxy)ethoxy)propanamido)benzamide |
| CPD-324 | 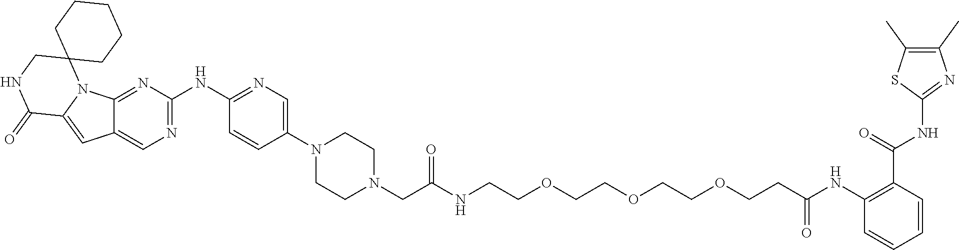 [SMILES: Cc1nc(NC(=O)c2ccccc2NC(=O)CCOCCOCCOCCNC(=O)CN2CCN(c3ccc(Nc4ncc5cc6n(c5n4)C4(CCCCC4)CNC6=O)nc3)CC2)sc1C] N-(4,5-dimethylthiazol-2-yl)-2-(2-oxo-1-(4-(6-((6′-oxo-7′,8′-dihydro-6′H- spiro[cyclohexane-1,9′-pyrazino[1′,2′:1,5]pyrrolo[2,3-d]pyrimidin]-2′- yl)amino)pyridin-3-yl)piperazin-1-yl)-6,9,12-trioxa-3-azapentadecan-15- amido)benzamide |
| CPD-325 | 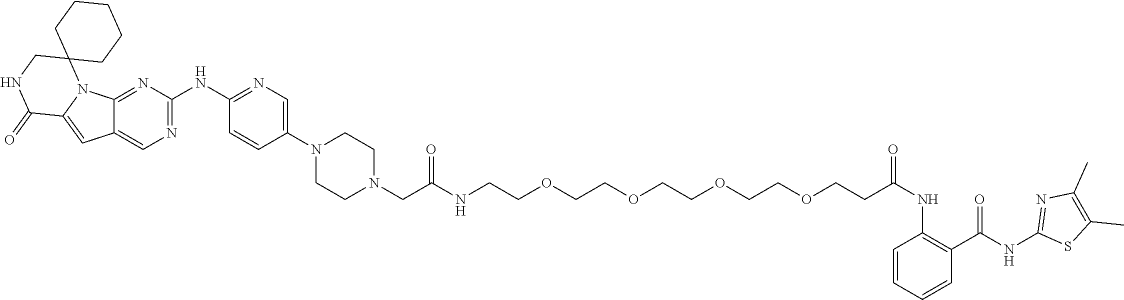 [SMILES: Cc1nc(NC(=O)c2ccccc2NC(=O)CCOCCOCCOCCOCCNC(=O)CN2CCN(c3ccc(Nc4ncc5cc6n(c5n4)C4(CCCCC4)CNC6=O)nc3)CC2)sc1C] N-(4,5-dimethylthiazol-2-yl)-2-(2-oxo-1-(4-(6-((6′-oxo-7′,8′-dihydro-6′H- spiro[cyclohexane-1,9′-pyrazino[1′,2′:1,5]pyrrolo[2,3-d]pyrimidin]-2′- yl)amino)pyridin-3-yl)piperazin-1-yl)-6,9,12,15-tetraoxa-3-azaoctadecan-18- amido)benzamide |
| CPD-326 | 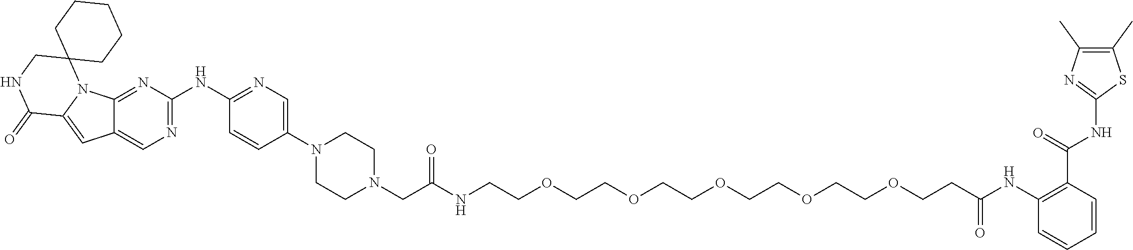 [SMILES: Cc1nc(NC(=O)c2ccccc2NC(=O)CCOCCOCCOCCOCCOCCNC(=O)CN2CCN(c3ccc(Nc4ncc5cc6n(c5n4)C4(CCCCC4)CNC6=O)nc3)CC2)sc1C] N-(4,5-dimethylthiazol-2-yl)-2-(2-oxo-1-(4-(6-((6′-oxo-7′,8′-dihydro-6′H- spiro[cyclohexane-1,9′-pyrazino[1′,2′:1,5]pyrrolo[2,3-d]pyrimidin]-2′- yl)amino)pyridin-3-yl)piperazin-1-yl)-6,9,12,15,18-pentaoxa-3-azahenicosan-21- amido)benzamide |
| CPD-327 | 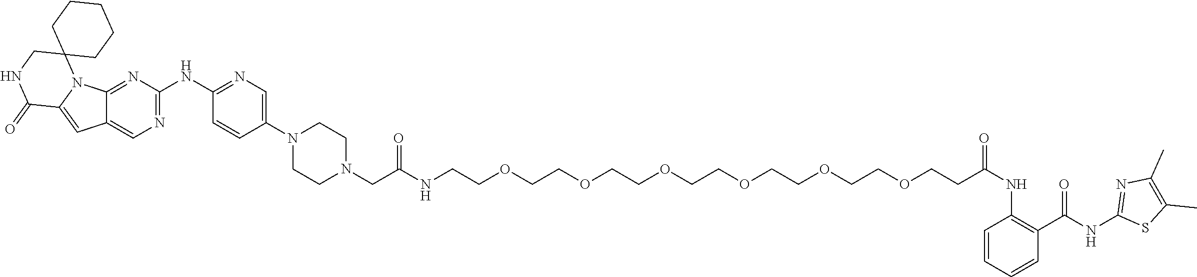 [SMILES: Cc1nc(NC(=O)c2ccccc2NC(=O)CCOCCOCCOCCOCCOCCOCCNC(=O)CN2CCN(c3ccc(Nc4ncc5cc6n(c5n4)C4(CCCCC4)CNC6=O)nc3)CC2)sc1C] N-(4,5-dimethylthiazol-2-yl)-2-(2-oxo-1-(4-(6-((6′-oxo-7′,8′-dihydro-6′H- spiro[cyclohexane-1,9′-pyrazino[1′,2′:1,5]pyrrolo[2,3-d]pyrimidin]-2′- yl)amino)pyridin-3-yl)piperazin-1-yl)-6,9,12,15,18,21-hexaoxa-3-azatetracosan-24- amido)benzamide |
| CPD-328 | 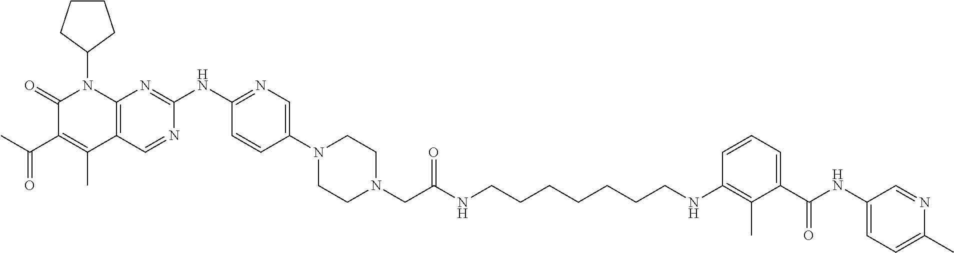 [SMILES: CC(=O)c1c(C)c2cnc(Nc3ccc(N4CCN(CC(=O)NCCCCCCCNc5cccc(C(=O)Nc6ccc(C)nc6)c5C)CC4)cn3)nc2n(C2CCCC2)c1=O] 3-((7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptyl)amino)-2- methyl-N-(6-methylpyridin-3-yl)benzamide |
| CPD-329 | 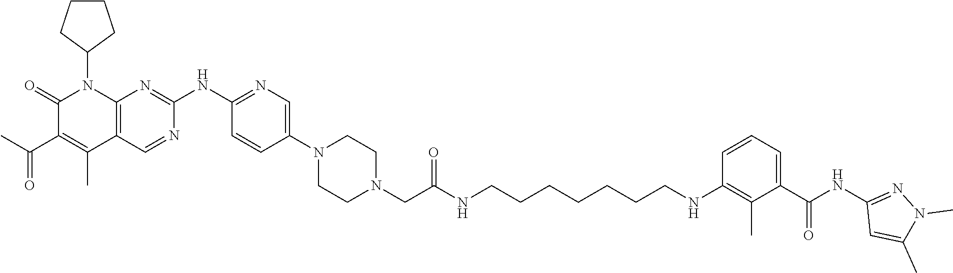 [SMILES: CC(=O)c1c(C)c2cnc(Nc3ccc(N4CCN(CC(=O)NCCCCCCCNc5cccc(C(=O)Nc6cc(C)n(C)n6)c5C)CC4)cn3)nc2n(C2CCCC2)c1=O] 3-((7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptyl)amino)-N-(1,5- dimethyl-1H-pyrazol-3-yl)-2-methylbenzamide |
| CPD-330 | 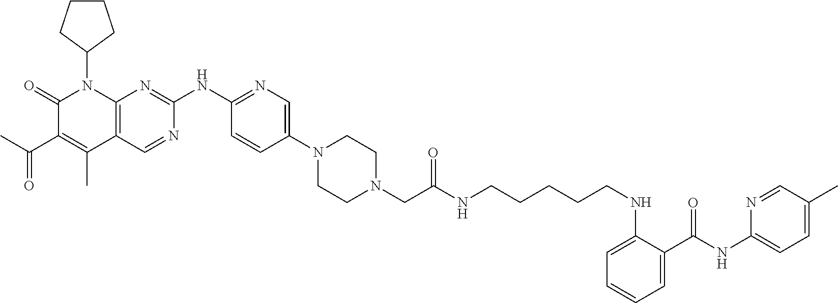 [SMILES: CC(=O)c1c(C)c2cnc(Nc3ccc(N4CCN(CC(=O)NCCCCCNc5ccccc5C(=O)Nc5ccc(C)cn5)CC4)cn3)nc2n(C2CCCC2)c1=O] 2-((5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentyl)amino)-N-(5- methylpyridin-2-yl)benzamide |
| CPD-331 | 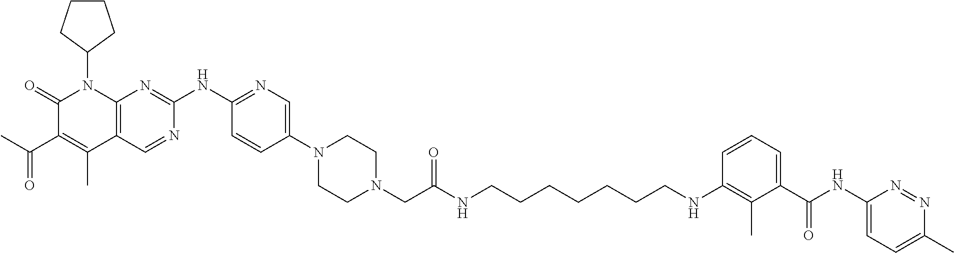 [SMILES: CC(=O)c1c(C)c2cnc(Nc3ccc(N4CCN(CC(=O)NCCCCCCCNc5cccc(C(=O)Nc6ccc(C)nn6)c5C)CC4)cn3)nc2n(C2CCCC2)c1=O] 3-((7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptyl)amino)-2- methyl-N-(6-methylpyridazin-3-yl)benzamide |
| CPD-332 | 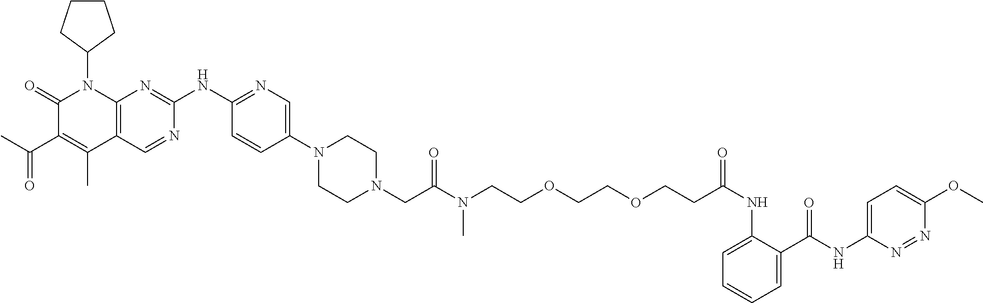 [SMILES: COc1ccc(NC(=O)c2ccccc2NC(=O)CCOCCOCCN(C)C(=O)CN2CCN(c3ccc(Nc4ncc5c(C)c(C(C)=O)c(=O)n(C6CCCC6)c5n4)nc3)CC2)nn1] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-N- methylacetamido)ethoxy)ethoxy)propanamido)-N-(6-methoxypyridazin-3- yl)benzamide |
| CPD-333 | 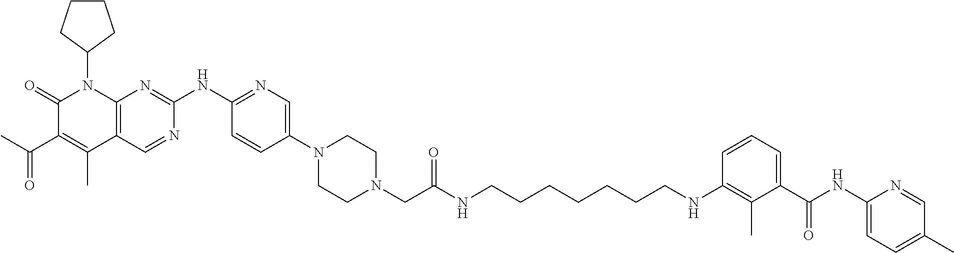 [SMILES: CC(=O)c1c(C)c2cnc(Nc3ccc(N4CCN(CC(=O)NCCCCCCCNc5cccc(C(=O)Nc6ccc(C)cn6)c5C)CC4)cn3)nc2n(C2CCCC2)c1=O] 3-((7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptyl)amino)-2- methyl-N-(5-methylpyridin-2-yl)benzamide |
| CPD-334 | 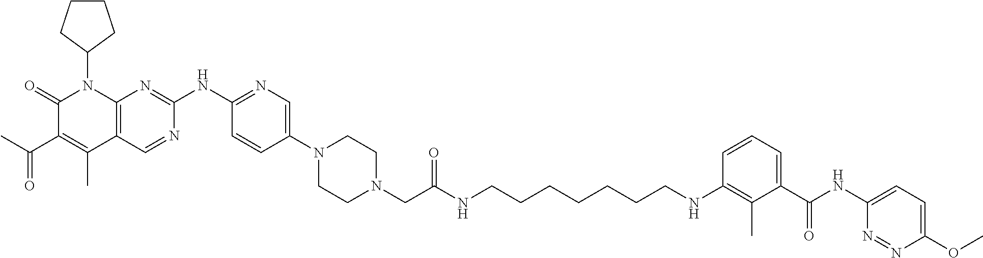 [SMILES: COc1ccc(NC(=O)c2cccc(NCCCCCCCNC(=O)CN3CCN(c4ccc(Nc5ncc6c(C)c(C(C)=O)c(=O)n(C7CCCC7)c6n5)nc4)CC3)c2C)nn1] 3-((7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptyl)amino)-N-(6- methoxypyridazin-3-yl)-2-methylbenzamide |
| CPD-335 | 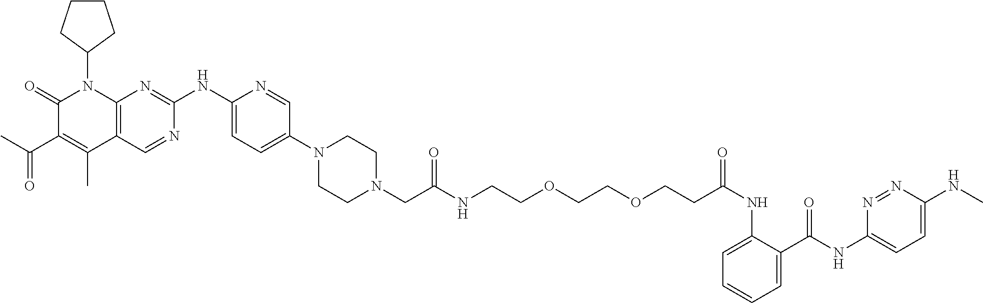 [SMILES: CNc1ccc(NC(=O)c2ccccc2NC(=O)CCOCCOCCNC(=O)CN2CCN(c3ccc(Nc4ncc5c(C)c(C(C)=O)c(=O)n(C6CCCC6)c5n4)nc3)CC2)nn1] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(6-(methylamino)pyridazin-3- yl)benzamide |
| CPD-336 | 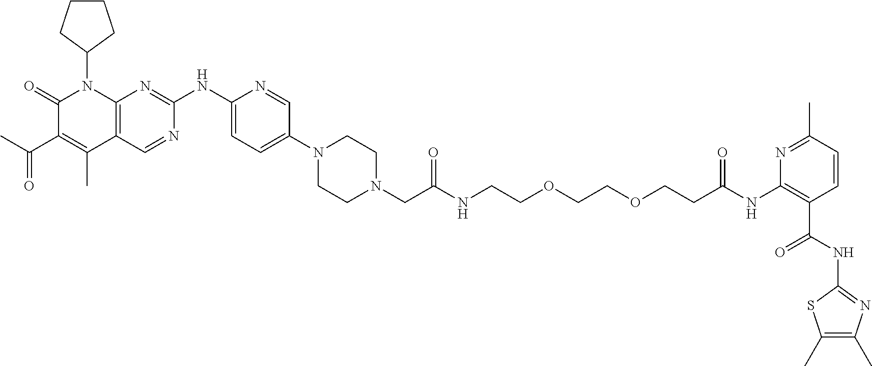 [SMILES: CC(=O)c1c(C)c2cnc(Nc3ccc(N4CCN(CC(=O)NCCOCCOCCC(=O)Nc5nc(C)ccc5C(=O)Nc5nc(C)c(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(4,5-dimethylthiazol-2-yl)-6- methylnicotinamide |
| CPD-337 | 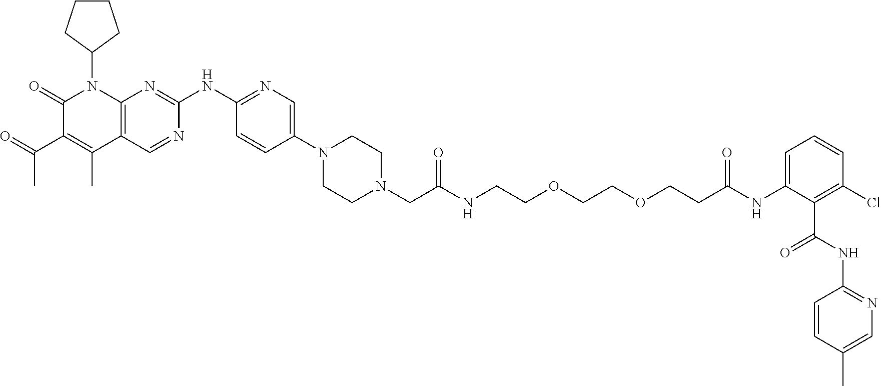 [SMILES: CC(=O)c1c(C)c2cnc(Nc3ccc(N4CCN(CC(=O)NCCOCCOCCC(=O)Nc5cccc(Cl)c5C(=O)Nc5ccc(C)cn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-6-chloro-N-(5-methylpyridin-2- yl)benzamide |
| CPD-338 | 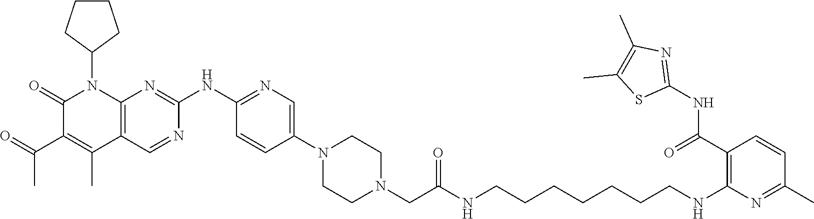 [SMILES: CC(=O)c1c(C)c2cnc(Nc3ccc(N4CCN(CC(=O)NCCCCCCCNc5nc(C)ccc5C(=O)Nc5nc(C)c(C)s5)CC4)cn3)nc2n(C2CCCC2)c1=O] 2-((7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptyl)amino)-N-(4,5- dimethylthiazol-2-yl)-6-methylnicotinamide |
| CPD-339 | 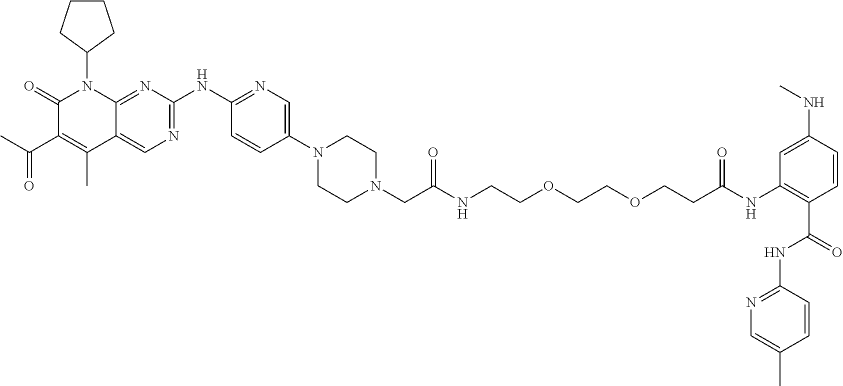 [SMILES: CNc1ccc(C(=O)Nc2ccc(C)cn2)c(NC(=O)CCOCCOCCNC(=O)CN2CCN(c3ccc(Nc4ncc5c(C)c(C(C)=O)c(=O)n(C6CCCC6)c5n4)nc3)CC2)c1] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-4-(methylamino)-N-(5-methylpyridin-2- yl)benzamide |
| CPD-340 | 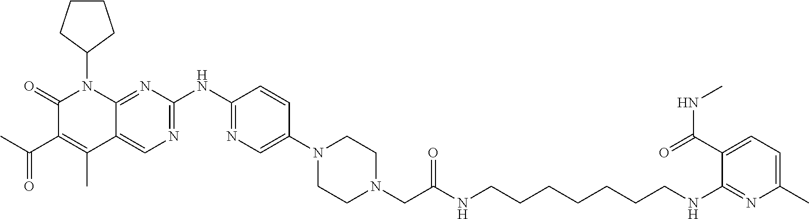 [SMILES: CNC(=O)c1ccc(C)nc1NCCCCCCCNC(=O)CN1CCN(c2ccc(Nc3ncc4c(C)c(C(C)=O)c(=O)n(C5CCCC5)c4n3)nc2)CC1] 2-((7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptyl)amino)-N,6- dimethylnicotinamide |
| CPD-341 | 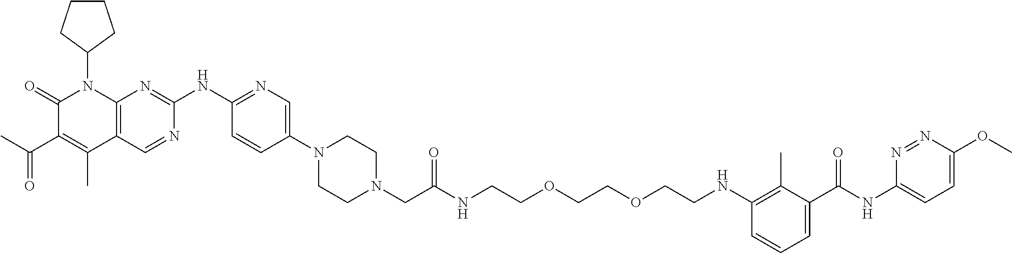 [SMILES: COc1ccc(NC(=O)c2cccc(NCCOCCOCCNC(=O)CN3CCN(c4ccc(Nc5ncc6c(C)c(C(C)=O)c(=O)n(C7CCCC7)c6n5)nc4)CC3)c2C)nn1] 3-((2-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)ethyl)amino)-N-(6-methoxypyridazin-3-yl)-2- methylbenzamide |
| CPD-342 | 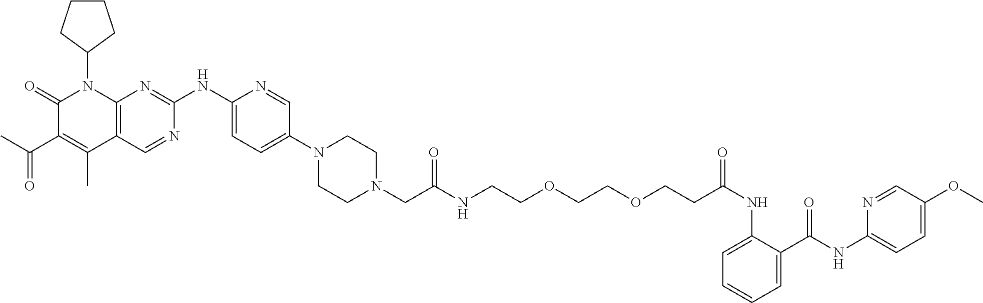 [SMILES: COc1ccc(NC(=O)c2ccccc2NC(=O)CCOCCOCCNC(=O)CN2CCN(c3ccc(Nc4ncc5c(C)c(C(C)=O)c(=O)n(C6CCCC6)c5n4)nc3)CC2)nc1] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methoxypyridin-2-yl)benzamide |
| CPD-343 | 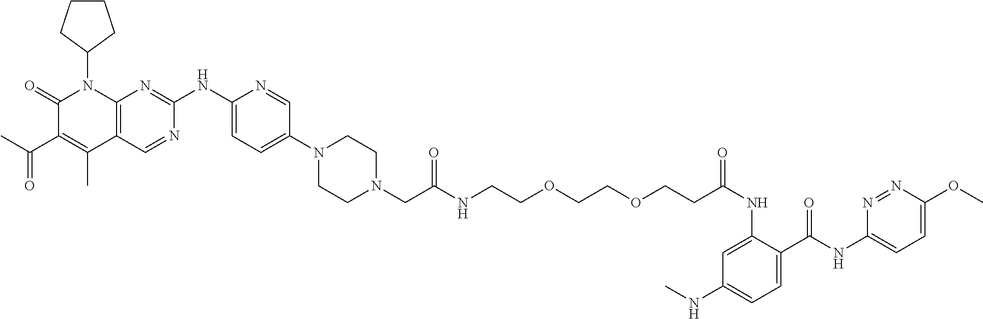 [SMILES: CNc1ccc(C(=O)Nc2ccc(OC)nn2)c(NC(=O)CCOCCOCCNC(=O)CN2CCN(c3ccc(Nc4ncc5c(C)c(C(C)=O)c(=O)n(C6CCCC6)c5n4)nc3)CC2)c1] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(6-methoxypyridazin-3-yl)-4- (methylamino)benzamide |
| CPD-344 | 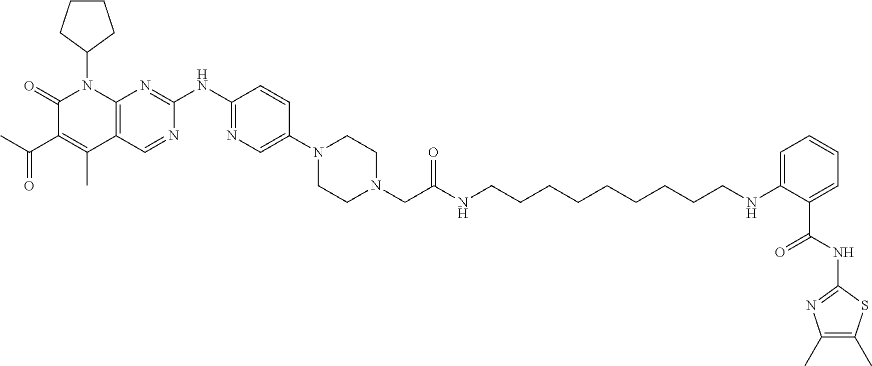 [SMILES: CC(=O)c1c(C)c2cnc(Nc3ccc(N4CCN(CC(=O)NCCCCCCCCCNc5ccccc5C(=O)Nc5nc(C)c(C)s5)CC4)cn3)nc2n(C2CCCC2)c1=O] 2-((9-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)nonyl)amino)-N-(4,5- dimethylthiazol-2-yl)benzamide |
| CPD-345 | 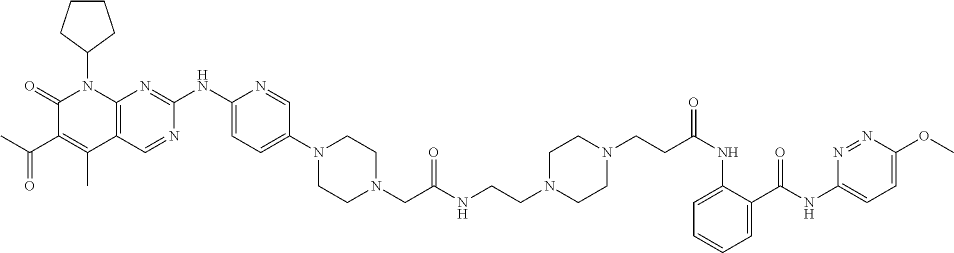 [SMILES: COc1ccc(NC(=O)c2ccccc2NC(=O)CCN2CCN(CCNC(=O)CN3CCN(c4ccc(Nc5ncc6c(C)c(C(C)=O)c(=O)n(C7CCCC7)c6n5)nc4)CC3)CC2)nn1] 2-(3-(4-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)ethyl)piperazin-1- yl)propanamido)-N-(6-methoxypyridazin-3-yl)benzamide |
| CPD-346 | 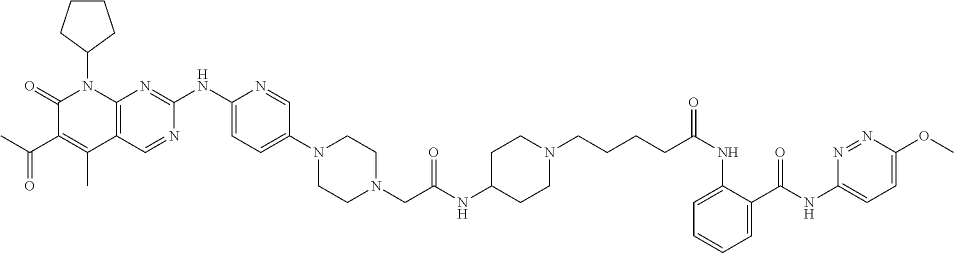 [SMILES: COc1ccc(NC(=O)c2ccccc2NC(=O)CCCCN2CCC(NC(=O)CN3CCN(c4ccc(Nc5ncc6c(C)c(C(C)=O)c(=O)n(C7CCCC7)c6n5)nc4)CC3)CC2)nn1] 2-(5-(4-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)piperidin-1- yl)pentanamido)-N-(6-methoxypyridazin-3-yl)benzamide |
| CPD-347 | 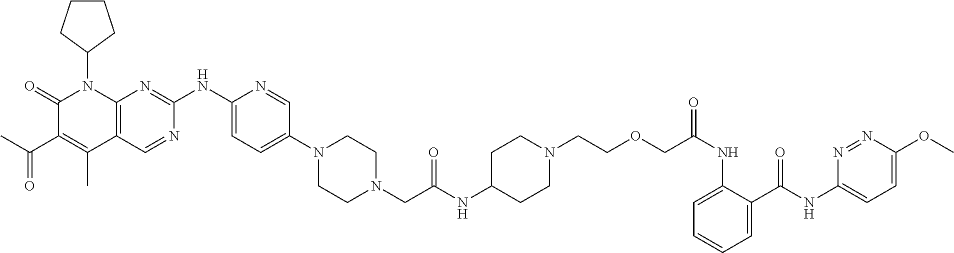 [SMILES: COc1ccc(NC(=O)c2ccccc2NC(=O)COCCN2CCC(NC(=O)CN3CCN(c4ccc(Nc5ncc6c(C)c(C(C)=O)c(=O)n(C7CCCC7)c6n5)nc4)CC3)CC2)nn1] 2-(2-(2-(4-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)piperidin-1- yl)ethoxy)acetamido)-N-(6-methoxypyridazin-3-yl)benzamide |
| CPD-348 | 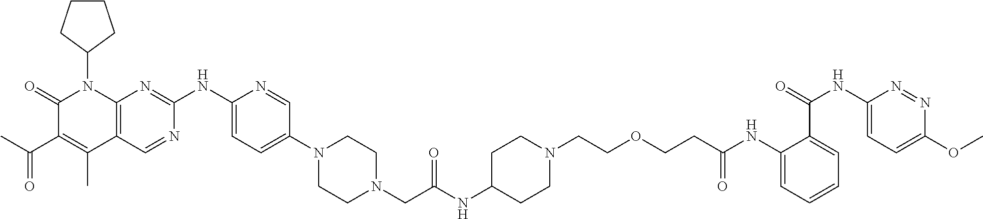 [SMILES: COc1ccc(NC(=O)c2ccccc2NC(=O)CCOCCN2CCC(NC(=O)CN3CCN(c4ccc(Nc5ncc6c(C)c(C(C)=O)c(=O)n(C7CCCC7)c6n5)nc4)CC3)CC2)nn1] 2-(3-(2-(4-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)piperidin-1- yl)ethoxy)propanamido)-N-(6-methoxypyridazin-3-yl)benzamide |
| CPD-349 | 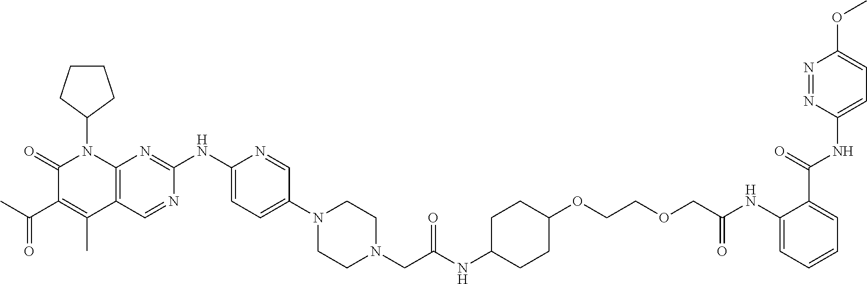 [SMILES: COc1ccc(NC(=O)c2ccccc2NC(=O)COCCOC2CCC(NC(=O)CN3CCN(c4ccc(Nc5ncc6c(C)c(C(C)=O)c(=O)n(C7CCCC7)c6n5)nc4)CC3)CC2)nn1] 2-(2-(2-((4-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)cyclohexyl)oxy)ethoxy)acetamido)-N-(6-methoxypyridazin-3- yl)benzamide |
| CPD-350 | 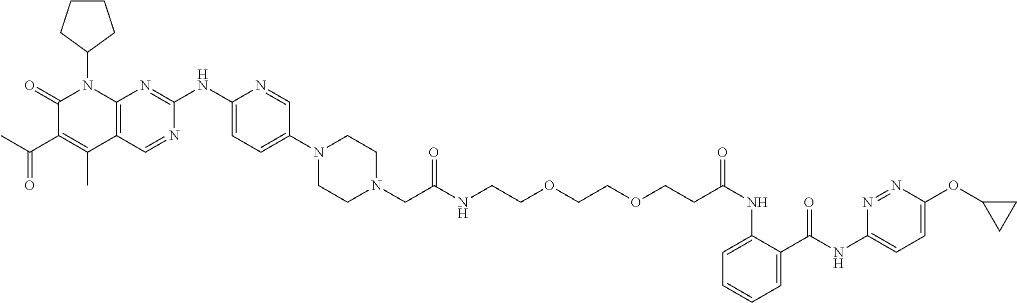 [SMILES: CC(=O)c1c(C)c2cnc(Nc3ccc(N4CCN(CC(=O)NCCOCCOCCC(=O)Nc5ccccc5C(=O)Nc5ccc(OC6CC6)nn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(6-cyclopropoxypyridazin-3- yl)benzamide |
| CPD-351 | 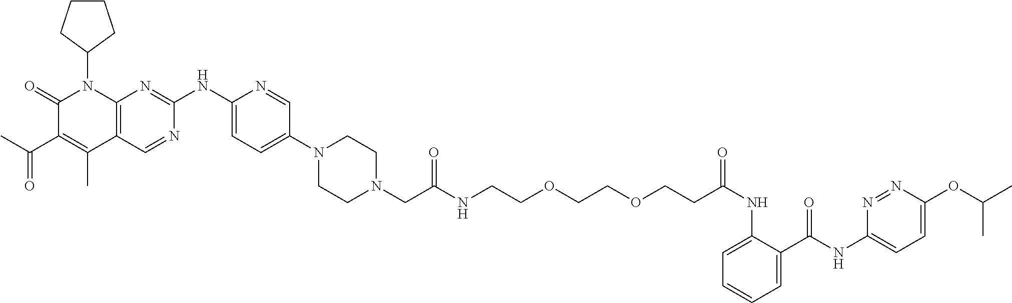 [SMILES: CC(=O)c1c(C)c2cnc(Nc3ccc(N4CCN(CC(=O)NCCOCCOCCC(=O)Nc5ccccc5C(=O)Nc5ccc(OC(C)C)nn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(6-isopropoxypyridazin-3-yl)benzamide |
| CPD-352 | 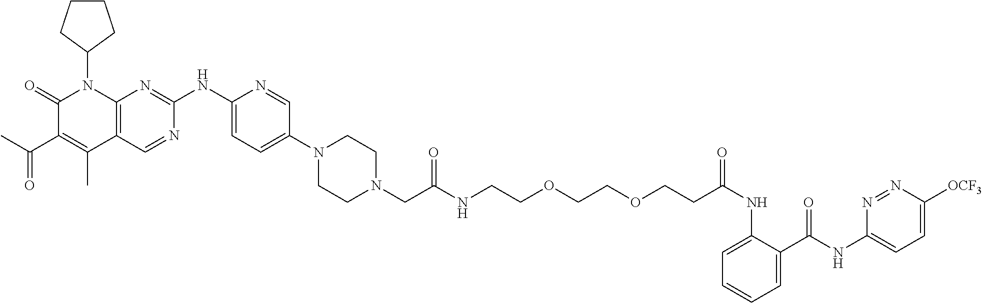 [SMILES: CC(=O)c1c(C)c2cnc(Nc3ccc(N4CCN(CC(=O)NCCOCCOCCC(=O)Nc5ccccc5C(=O)Nc5ccc(OC(F)(F)F)nn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(6-(trifluoromethoxy)pyridazin-3- yl)benzamide |
| CPD-353 | 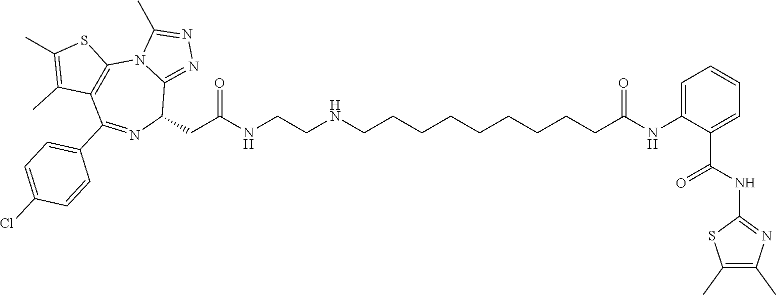 [SMILES: Cc1nc(NC(=O)c2ccccc2NC(=O)CCCCCCCCCNCCNC(=O)C[C@@H]2N=C(c3ccc(Cl)cc3)c3c(sc(C)c3C)-n3c(C)nnc32)sc1C] (S)-2-(10-((2-(2-(4-(4-chlorophenyl)-2,3,9-trimethyl-6H-thieno[3,2- f][1,2,4]triazolo[4,3-a][1,4]diazepin-6-yl)acetamido)ethyl)amino)decanamido)-N- (4,5-dimethylthiazol-2-yl)benzamide |
| CPD-354 | 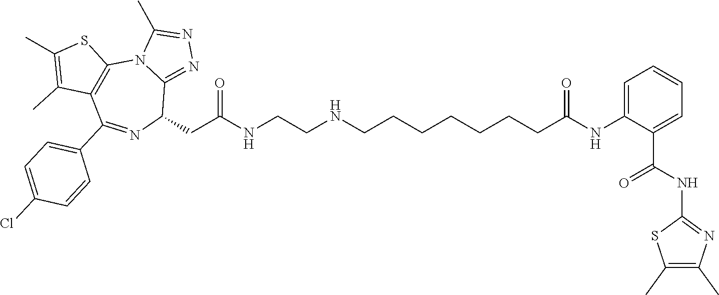 [SMILES: Cc1nc(NC(=O)c2ccccc2NC(=O)CCCCCCCNCCNC(=O)C[C@@H]2N=C(c3ccc(Cl)cc3)c3c(sc(C)c3C)-n3c(C)nnc32)sc1C] (S)-2-(8-((2-(2-(4-(4-chlorophenyl)-2,3,9-trimethyl-6H-thieno[3,2- f][1,2,4]triazolo[4,3-a][1,4]diazepin-6-yl)acetamido)ethyl)amino)octanamido)-N-(4,5- dimethylthiazol-2-yl)benzamide |
| CPD-355 | 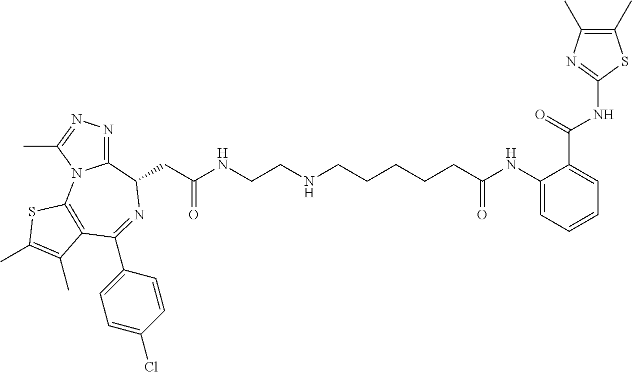 (S)-2-(6-((2-(2-(4-(4-chlorophenyl)-2,3,9-trimethyl-6H-thieno[3,2- f][1,2,4]triazolo[4,3-a][1,4]diazepin-6-yl)acetamido)ethyl)amino)hexanamido)-N- (4,5-dimethylthiazol-2-yl)benzamide |
| CPD-356 | 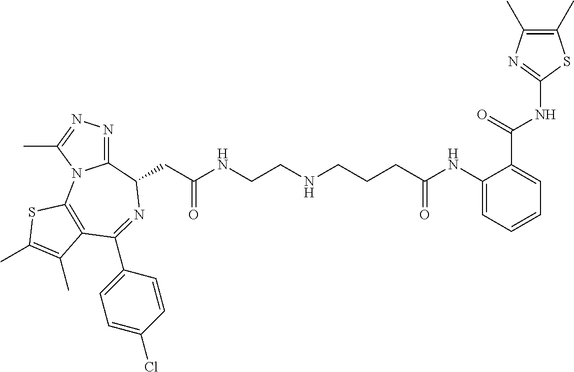 (S)-2-(4-((2-(2-(4-(4-chlorophenyl)-2,3,9-trimethyl-6H-thieno[3,2- f][1,2,4]triazolo[4,3-a][1,4]diazepin-6-yl)acetamido)ethyl)amino)butanamido)-N-(4,5- dimethylthiazol-2-yl)benzamide |
| CPD-357 | 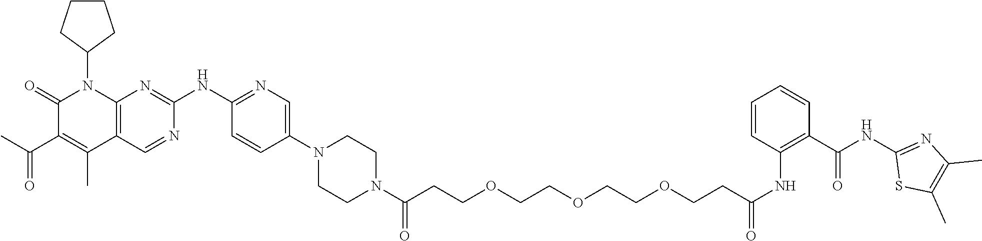 [SMILES: CC(=O)c1c(C)c2cnc(Nc3ccc(N4CCN(C(=O)CCOCCOCCOCCC(=O)Nc5ccccc5C(=O)Nc5nc(C)c(C)s5)CC4)cn3)nc2n(C2CCCC2)c1=O] 2-(3-(2-(2-(3-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)-3- oxopropoxy)ethoxy)ethoxy)propanamido)-N-(4,5-dimethylthiazol-2-yl)benzamide |
| CPD-358 | 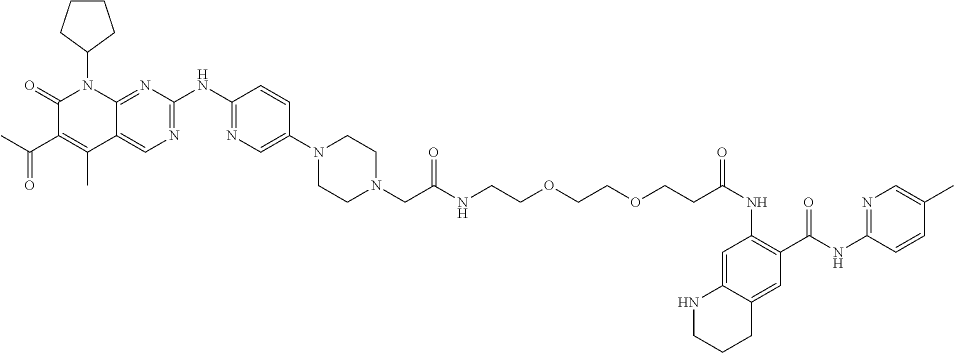 [SMILES: CC(=O)c1c(C)c2cnc(Nc3ccc(N4CCN(CC(=O)NCCOCCOCCC(=O)Nc5cc6c(cc5C(=O)Nc5ccc(C)cn5)CCCN6)CC4)cn3)nc2n(C2CCCC2)c1=O] 7-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methylpyridin-2-yl)-1,2,3,4- tetrahydroquinoline-6-carboxamide |
| CPD-359 | 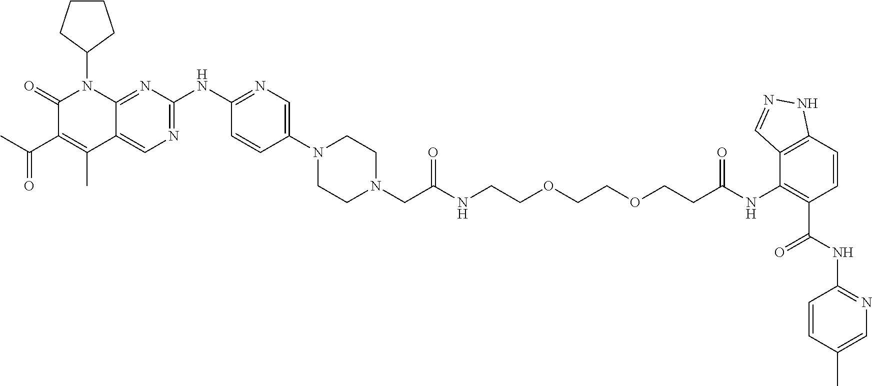 [SMILES: CC(=O)c1c(C)c2cnc(Nc3ccc(N4CCN(CC(=O)NCCOCCOCCC(=O)Nc5c(C(=O)Nc6ccc(C)cn6)ccc6[nH]ncc56)CC4)cn3)nc2n(C2CCCC2)c1=O] 4-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methylpyridin-2-yl)-1H-indazole-5- carboxamide |
| CPD-360 | 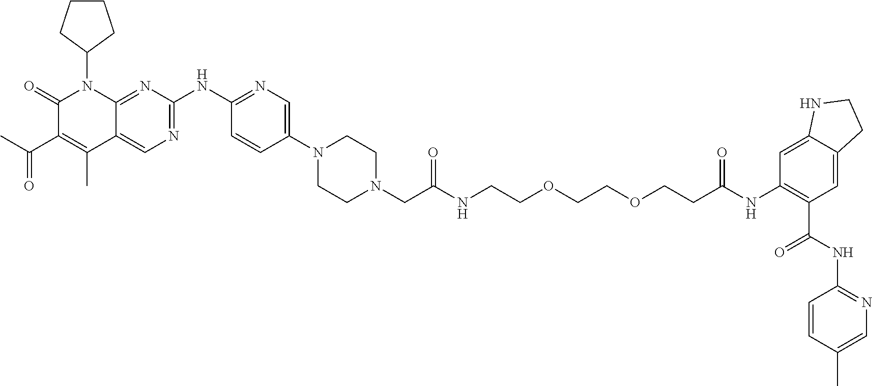 [SMILES: CC(=O)c1c(C)c2cnc(Nc3ccc(N4CCN(CC(=O)NCCOCCOCCC(=O)Nc5cc6c(cc5C(=O)Nc5ccc(C)cn5)CCN6)CC4)cn3)nc2n(C2CCCC2)c1=O] 6-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methylpyridin-2-yl)indoline-5- carboxamide |
| CPD-361 | 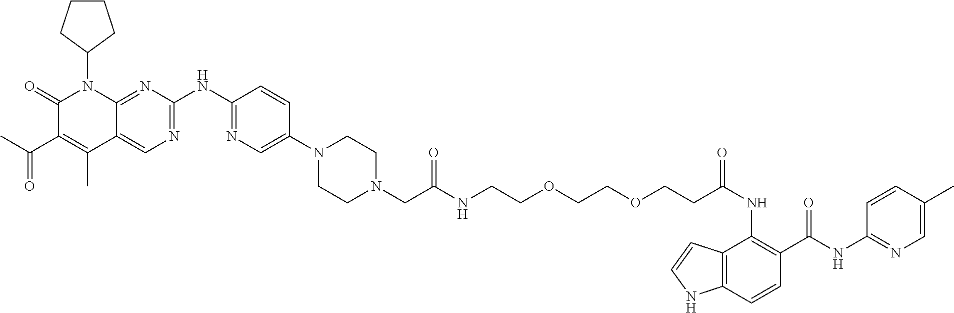 [SMILES: CC(=O)c1c(C)c2cnc(Nc3ccc(N4CCN(CC(=O)NCCOCCOCCC(=O)Nc5c(C(=O)Nc6ccc(C)cn6)ccc6[nH]ccc56)CC4)cn3)nc2n(C2CCCC2)c1=O] 4-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methylpyridin-2-yl)-1H-indole-5- carboxamide |
| CPD-362 | 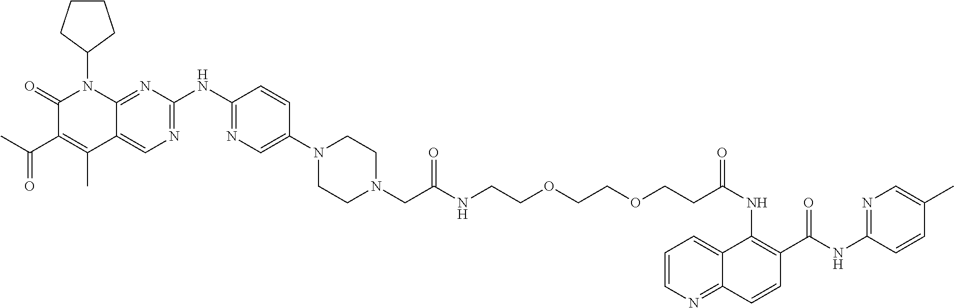 [SMILES: CC(=O)c1c(C)c2cnc(Nc3ccc(N4CCN(CC(=O)NCCOCCOCCC(=O)Nc5c(C(=O)Nc6ccc(C)cn6)ccc6ncccc56)CC4)cn3)nc2n(C2CCCC2)c1=O] 5-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methylpyridin-2-yl)quinoline-6- carboxamide |
| CPD-363 | 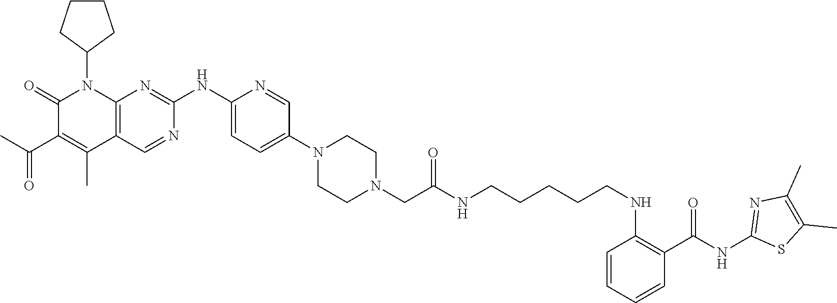 [SMILES: CC(=O)c1c(C)c2cnc(Nc3ccc(N4CCN(CC(=O)NCCCCCNc5ccccc5C(=O)Nc5nc(C)c(C)s5)CC4)cn3)nc2n(C2CCCC2)c1=O] 2-((5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentyl)amino)-N-(4,5- dimethylthiazol-2-yl)benzamide |
| CPD-364 | 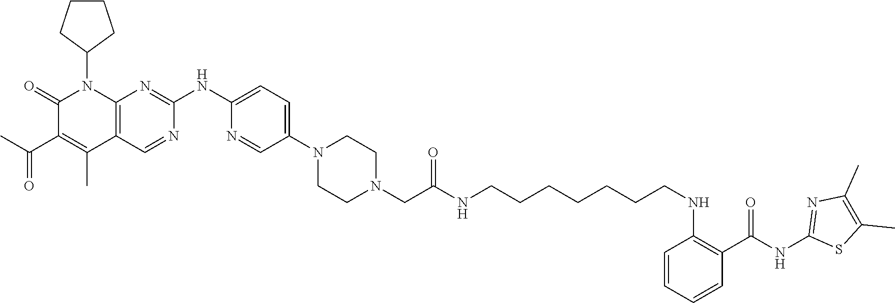 [SMILES: CC(=O)c1c(C)c2cnc(Nc3ccc(N4CCN(CC(=O)NCCCCCCCNc5ccccc5C(=O)Nc5nc(C)c(C)s5)CC4)cn3)nc2n(C2CCCC2)c1=O] 2-((7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptyl)amino)-N-(4,5- dimethylthiazol-2-yl)benzamide |
| CPD-365 | 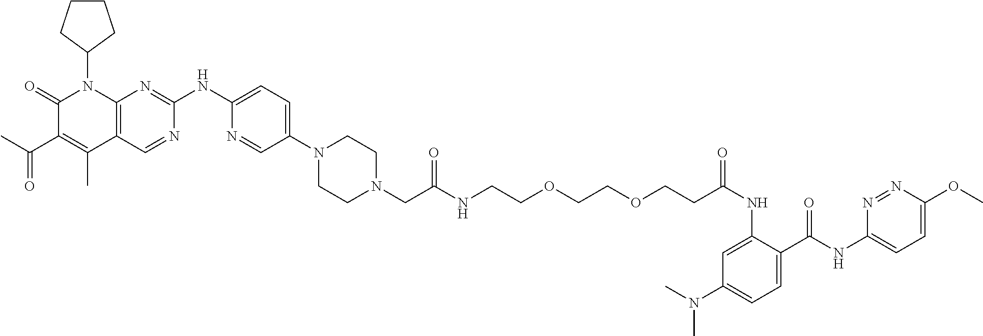 [SMILES: COc1ccc(NC(=O)c2ccc(N(C)C)cc2NC(=O)CCOCCOCCNC(=O)CN2CCN(c3ccc(Nc4ncc5c(C)c(C(C)=O)c(=O)n(C6CCCC6)c5n4)nc3)CC2)nn1] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-4-(dimethylamino)-N-(6- methoxypyridazin-3-yl)benzamide |
| CPD-366 | 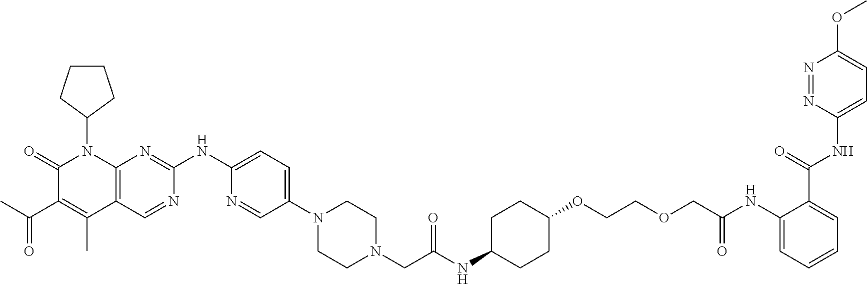 [SMILES: COc1ccc(NC(=O)c2ccccc2NC(=O)COCCO[C@H]2CC[C@H](NC(=O)CN3CCN(c4ccc(Nc5ncc6c(C)c(C(C)=O)c(=O)n(C7CCCC7)c6n5)nc4)CC3)CC2)nn1] 2-(2-(2-((trans-4-(2-(4-(6-((6-acetyl-8-cyclopentyl-5-methyl-7-oxo-7,8- dihydropyrido[2,3-d]pyrimidin-2-yl)amino)pyridin-3-yl)piperazin-1- yl)acetamido)cyclohexyl)oxy)ethoxy)acetamido)-N-(6-methoxypyridazin-3- yl)benzamide |
| CPD-367 | 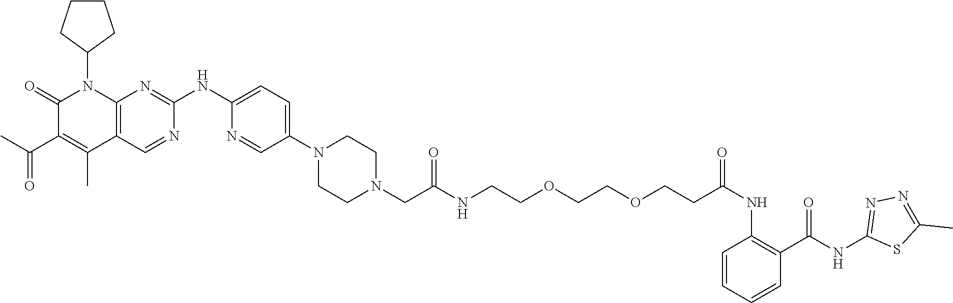 [SMILES: CC(=O)c1c(C)c2cnc(Nc3ccc(N4CCN(CC(=O)NCCOCCOCCC(=O)Nc5ccccc5C(=O)Nc5nnc(C)s5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(5-methyl-1,3,4-thiadiazol-2- yl)benzamide |
| CPD-368 | 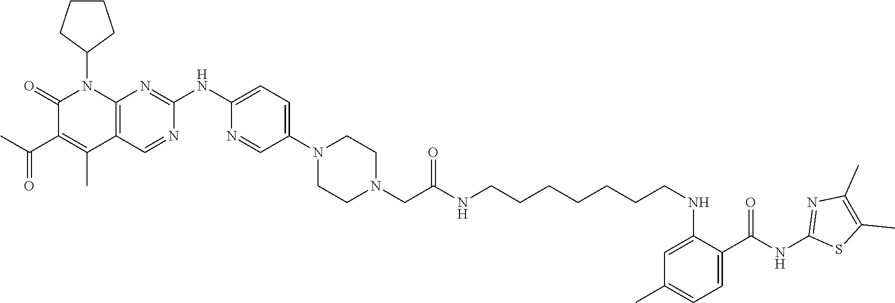 [SMILES: CC(=O)c1c(C)c2cnc(Nc3ccc(N4CCN(CC(=O)NCCCCCCCNc5cc(C)ccc5C(=O)Nc5nc(C)c(C)s5)CC4)cn3)nc2n(C2CCCC2)c1=O] 2-((7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptyl)amino)-N-(4,5- dimethylthiazol-2-yl)-4-methylbenzamide |
| CPD-369 | 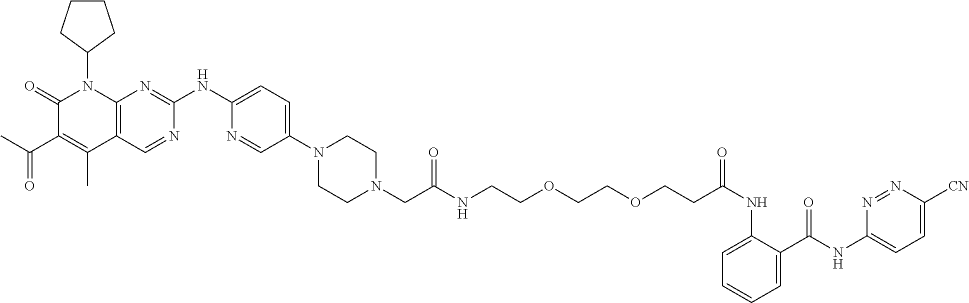 [SMILES: CC(=O)c1c(C)c2cnc(Nc3ccc(N4CCN(CC(=O)NCCOCCOCCC(=O)Nc5ccccc5C(=O)Nc5ccc(C#N)nn5)CC4)cn3)nc2n(C2CCCC2)c1=O] 2-(3-(2-(2-(2-(4-(6-((6-acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)propanamido)-N-(6-cyanopyridazin-3-yl)benzamide |
| CPD-370 | 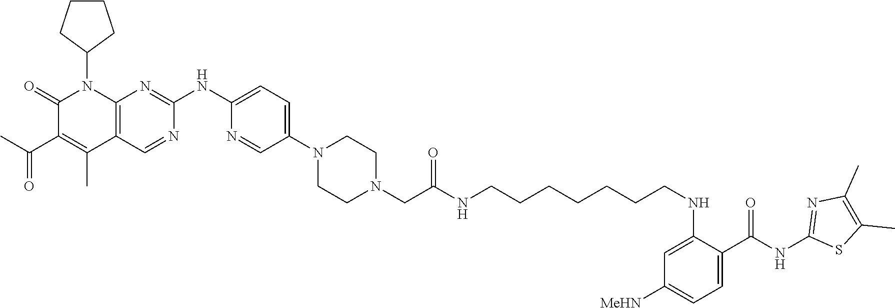 [SMILES: CNc1ccc(C(=O)Nc2nc(C)c(C)s2)c(NCCCCCCCNC(=O)CN2CCN(c3ccc(Nc4ncc5c(C)c(C(C)=O)c(=O)n(C6CCCC6)c5n4)nc3)CC2)c1] 2-((7-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)heptyl)amino)-N-(4,5- dimethylthiazol-2-yl)-4-(methylamino)benzamide |
| CPD-371 | 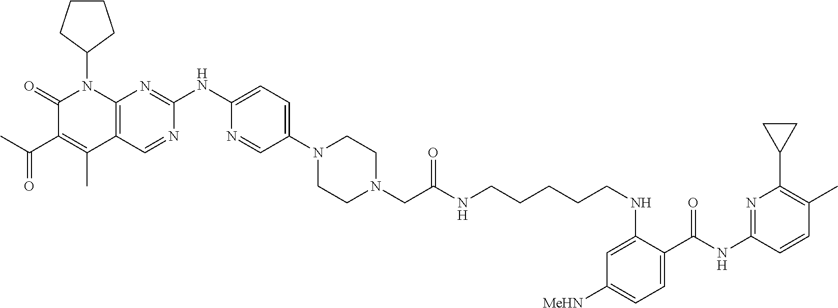 [SMILES: CNc1ccc(C(=O)Nc2ccc(C)c(C3CC3)n2)c(NCCCCCNC(=O)CN2CCN(c3ccc(Nc4ncc5c(C)c(C(C)=O)c(=O)n(C6CCCC6)c5n4)nc3)CC2)c1] 2-((5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentyl)amino)-N-(6- cyclopropyl-5-methylpyridin-2-yl)-4-(methylamino)benzamide |
| CPD-372 | 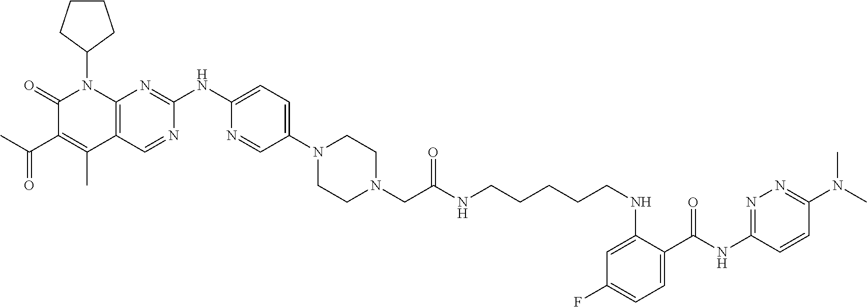 [SMILES: CC(=O)c1c(C)c2cnc(Nc3ccc(N4CCN(CC(=O)NCCCCCNc5cc(F)ccc5C(=O)Nc5ccc(N(C)C)nn5)CC4)cn3)nc2n(C2CCCC2)c1=O] 2-((5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentyl)amino)-N-(6- (dimethylamino)pyridazin-3-yl)-4-fluorobenzamide |
| CPD-373 | 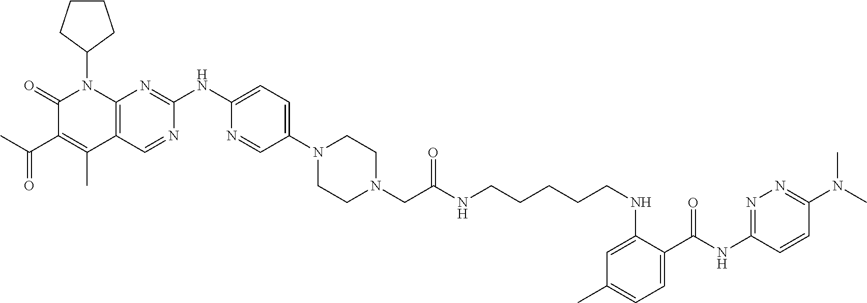 [SMILES: CC(=O)c1c(C)c2cnc(Nc3ccc(N4CCN(CC(=O)NCCCCCNc5cc(C)ccc5C(=O)Nc5ccc(N(C)C)nn5)CC4)cn3)nc2n(C2CCCC2)c1=O] 2-((5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentyl)amino)-N-(6- (dimethylamino)pyridazin-3-yl)-4-methylbenzamide |
| CPD-374 | 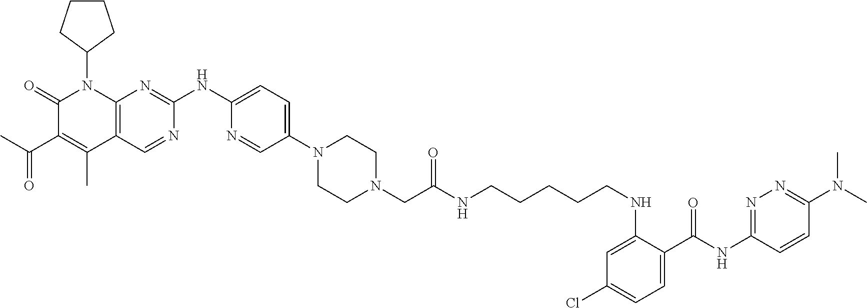 [SMILES: CC(=O)c1c(C)c2cnc(Nc3ccc(N4CCN(CC(=O)NCCCCCNc5cc(Cl)ccc5C(=O)Nc5ccc(N(C)C)nn5)CC4)cn3)nc2n(C2CCCC2)c1=O] 2-((5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentyl)amino)-4- chloro-N-(6-(dimethylamino)pyridazin-3-yl)benzamide |
| CPD-375 | 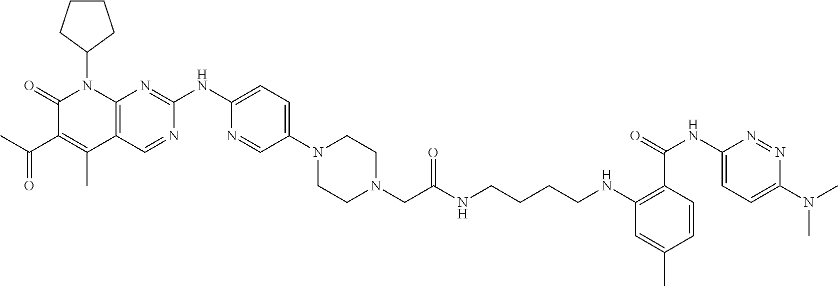 [SMILES: CC(=O)c1c(C)c2cnc(Nc3ccc(N4CCN(CC(=O)NCCCCNc5cc(C)ccc5C(=O)Nc5ccc(N(C)C)nn5)CC4)cn3)nc2n(C2CCCC2)c1=O] 2-((4-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)butyl)amino)-N-(6- (dimethylamino)pyridazin-3-yl)-4-methylbenzamide |
| CPD-376 | 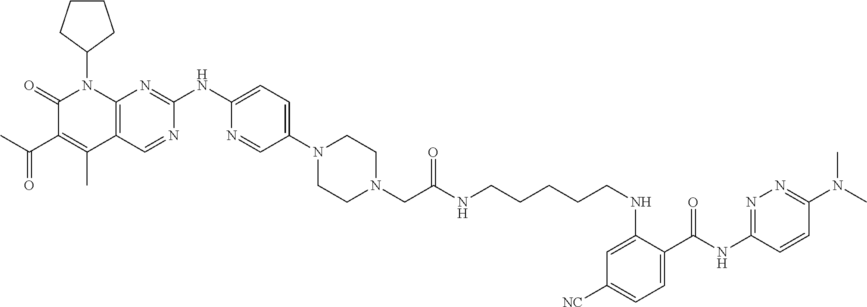 [SMILES: CC(=O)c1c(C)c2cnc(Nc3ccc(N4CCN(CC(=O)NCCCCCNc5cc(C#N)ccc5C(=O)Nc5ccc(N(C)C)nn5)CC4)cn3)nc2n(C2CCCC2)c1=O] 2-((5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentyl)amino)-4- cyano-N-(6-(dimethylamino)pyridazin-3-yl)benzamide |
| CPD-377 | 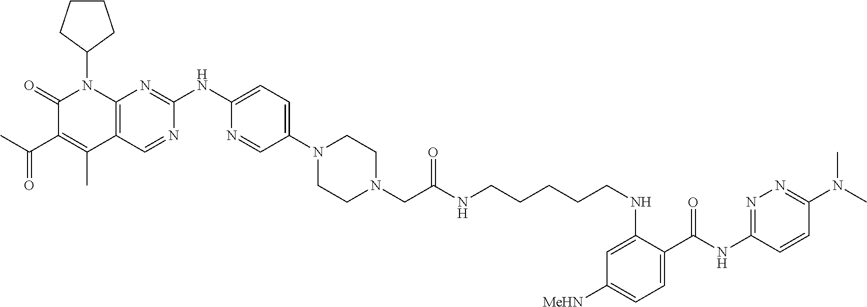 [SMILES: CNc1ccc(C(=O)Nc2ccc(N(C)C)nn2)c(NCCCCCNC(=O)CN2CCN(c3ccc(Nc4ncc5c(C)c(C(C)=O)c(=O)n(C6CCCC6)c5n4)nc3)CC2)c1] 2-((5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentyl)amino)-N-(6- (dimethylamino)pyridazin-3-yl)-4-(methylamino)benzamide |
| CPD-378 | 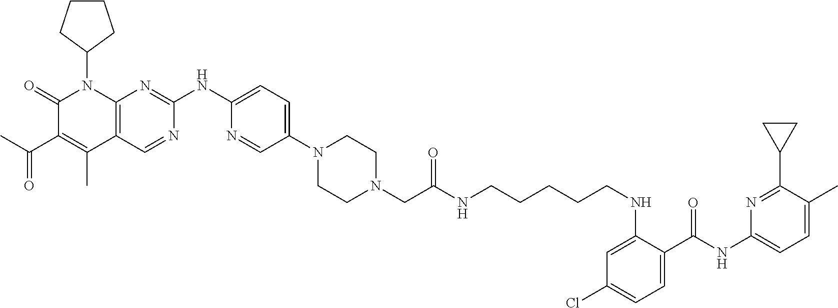 [SMILES: CC(=O)c1c(C)c2cnc(Nc3ccc(N4CCN(CC(=O)NCCCCCNc5cc(Cl)ccc5C(=O)Nc5ccc(C)c(C6CC6)n5)CC4)cn3)nc2n(C2CCCC2)c1=O] 2-((5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)pentyl)amino)-4- chloro-N-(6-cyclopropyl-5-methylpyridin-2-yl)benzamide |
| CPD-379 | 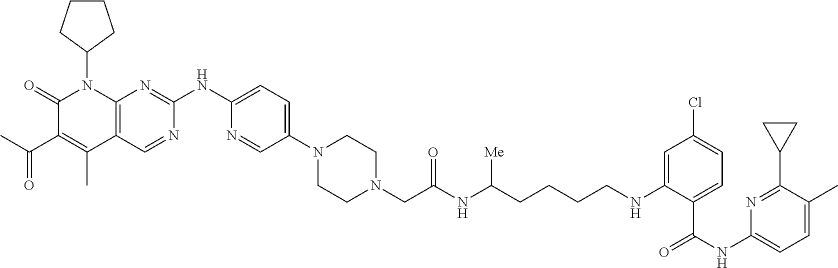 [SMILES: CC(=O)c1c(C)c2cnc(Nc3ccc(N4CCN(CC(=O)NC(C)CCCCNc5cc(Cl)ccc5C(=O)Nc5ccc(C)c(C6CC6)n5)CC4)cn3)nc2n(C2CCCC2)c1=O] 2-((5-(2-(4-(6-((6-acetyl-8-cyclopentyl-5-methyl-7-oxo-7,8-dihydropyrido[2,3- d]pyrimidin-2-yl)amino)pyridin-3-yl)piperazin-1-yl)acetamido)hexyl)amino)-4- chloro-N-(6-cyclopropyl-5-methylpyridin-2-yl)benzamide |
| CPD-380 | 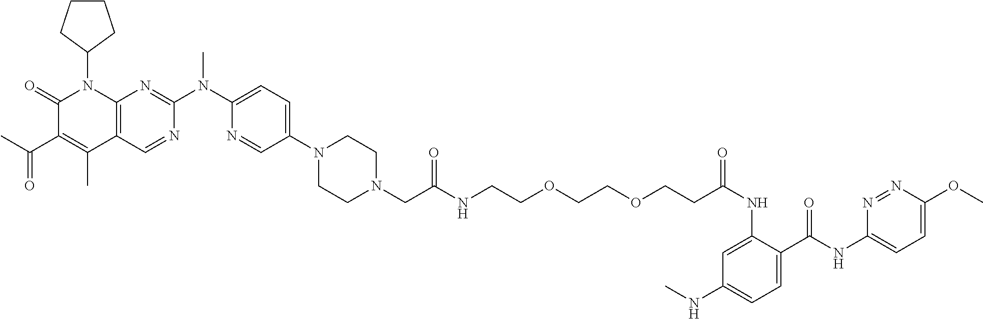 [SMILES: CNc1ccc(C(=O)Nc2ccc(OC)nn2)c(NC(=O)CCOCCOCCNC(=O)CN2CCN(c3ccc(N(C)c4ncc5c(C)c(C(C)=O)c(=O)n(C6CCCC6)c5n4)nc3)CC2)c1] 3-((2-(2-(2-(2-(4-(6-((6-Acetyl-8-cyclopentyl-5-methyl-7-oxo-7,8-dihydropyrido[2,3- d]pyrimidin-2-yl)amino)pyridin-3-yl)piperazin-1- yl)acetamido)ethoxy)ethoxy)ethyl)amino)-N-(6-methoxypyridazin-3-yl)-2- methylbenzamide |

In some embodiments, the heterobifunctional compound of Formula (I), or a pharmaceutically acceptable salt or solvate thereof, binds to a DDB1 protein through the DDB1 binding moiety. In some embodiments, the compound of Formula (I), or a pharmaceutically acceptable salt or solvate thereof, is bound to a DDB1 protein via the DDB1 binding moiety. In some embodiments, the heterobifunctional compound or the DDB1 binding moiety does not inhibit DDB1 function. For example, binding of DDB1 to the DDB1 binding moiety may, in some embodiments, not prevent or reduce associations between DDB1 and a cullin protein such as Cullin 4A or Cullin 4B. In some embodiments, a DDB1 binding moiety is a small molecule.

### Modified or Engineered Proteins

Disclosed herein, in some embodiments, are modified proteins such as in vivo modified proteins. In some embodiments, the in vivo modified protein comprises a DNA damage-binding protein 1 (DDB1) protein. In some embodiments, the DDB1 protein is bound to a ligand. In some embodiments, the ligand is a DDB1 ligand. In some embodiments, the DDB1 protein is directly bound to the ligand. In some embodiments, the binding between the DDB1 protein and the ligand is non-covalent. In some embodiments, the binding between the DDB1 protein and the ligand is covalent. The ligand may be any ligand described herein. In some embodiments, the ligand comprises a compound disclosed herein, or a salt or variant thereof. In some embodiments, the ligand comprises a DDB1 binding moiety such as a DDB1 binding moiety described herein. In some embodiments, the DDB1 ligand is a heterobifunctional compound comprising a DDB1 binding moiety covalently connected through a linker to a target protein binding moiety described herein. In some embodiments, a DDB1 protein is modified in vivo by being bound to a ligand administered to a subject.

A modified protein may include an engineered protein. Disclosed herein, in some embodiments, are engineered DDB1 proteins such as an in vivo engineered DDB1 protein. The engineered DDB1 protein may be bound to a ligand. The engineered DDB1 protein may bind to the ligand in vivo. For example, the ligand may be administered to a subject, and bind to a DDB1 protein or engineered DDB1 protein in vivo.

Disclosed herein, in some embodiments, are in vivo modified proteins. In some embodiments, the in vivo modified protein comprises a DDB1 protein directly bound to a ligand comprising a DDB1 binding moiety. In some embodiments, the in vivo modified protein comprises a DDB1 protein directly bound to a ligand, the ligand comprising a DDB1 binding moiety. In some embodiments, the in vivo modified protein comprises a DDB1 protein directly bound to a heterobifunctional compound, the heterobifunctional compound comprising a DDB1 binding moiety covalently connected through a linker to a target protein binding moiety.

Disclosed herein, in some embodiments, are in vivo modified proteins. In some embodiments, the ligand comprises a DDB1 binding moiety. In some embodiments, the ligand comprises a linker. In some embodiments, the ligand comprises a target protein binding moiety. In some embodiments, the DDB1 binding moiety is covalently connected to a linker. In some embodiments, the linker is further connected to a target protein binding moiety. In some embodiments, the DDB1 binding moiety is covalently connected through a linker to a target protein binding moiety. In some embodiments, the DDB1 binding moiety is covalently connected to a target protein binding moiety without a linker. In some embodiments, target protein binding moiety binds to a target protein such as a target protein described herein. In some embodiments, the ligand comprises a compound described herein. For example, the ligand may comprise a DDB1 binding moiety disclosed herein, or the ligand may comprise a linker disclosed herein, or the ligand may comprise a target protein binding moiety disclosed herein. In some embodiments, a linker is a bond. In some embodiments, the linker is more than just a bond. In some embodiments, the ligand is a small molecule. In some embodiments, the ligand is a heterobifunctional compound comprising a DDB1 binding moiety covalently connected through a linker to a target protein binding moiety.

Disclosed herein, in some embodiments, are in vivo modified proteins. In some embodiments, the DDB1 binding moiety is bound to a binding region on the DDB1 protein. In some embodiments, the binding region on the DDB1 protein comprises a beta propeller domain. In some embodiments, the beta propeller domain comprises a beta propeller C (BPC) domain. In some embodiments, the binding region on the DDB1 protein comprises a BPC domain. In some embodiments, the binding region on the DDB1 protein comprises a top face of the BPC domain. Disclosed herein, in some embodiments, are in vivo modified proteins. In some embodiments, the binding region on the DDB1 protein comprises one or more of the following DDB1 residues: ARG327, LEU328, PRO358, ILE359, VAL360, ASP361, GLY380, ALA381, PHE382, SER720, ARG722, LYS723, SER738, ILE740, GLU787, TYR812, LEU814, SER815, ALA834, VAL836, ALA841, ALA869, TYR871, SER872, MET910, LEU912, TYR913, LEU926, TRP953, SER955, ALA956, ASN970, ALA971, PHE972, PHE1003, ASN1005, VAL1006, or VAL1033. In some embodiments, one or more of the following DDB1 residues are involved in the non-covalent binding between the DDB1 protein and the ligand: ARG327, LEU328, PRO358, ILE359, VAL360, ASP361, GLY380, ALA381, PHE382, SER720, ARG722, LYS723, SER738, ILE740, GLU787, TYR812, LEU814, SER815, ALA834, VAL836, ALA841, ALA869, TYR871, SER872, MET910, LEU912, TYR913, LEU926, TRP953, SER955, ALA956, ASN970, ALA971, PHE972, PHE1003, ASN1005, VAL1006, or VAL1033. An in vivo engineered DDB1 protein may include a DDB1 protein bound to a ligand at any of the aforementioned residues.

Disclosed herein, in some embodiments, are in vivo modified proteins. In some embodiments, the binding region on the DDB1 protein comprises ARG327 of the DDB1 protein. In some embodiments, the binding region on the DDB1 protein comprises LEU328 of the DDB1 protein. In some embodiments, the binding region on the DDB1 protein comprises PRO358 of the DDB1 protein. In some embodiments, the binding region on the DDB1 protein comprises ILE359 of the DDB1 protein. In some embodiments, the binding region on the DDB1 protein comprises VAL360 of the DDB1 protein. In some embodiments, the binding region on the DDB1 protein comprises ASP361 of the DDB1 protein. In some embodiments, the binding region on the DDB1 protein comprises GLY380 of the DDB1 protein. In some embodiments, the binding region on the DDB1 protein comprises ALA381 of the DDB1 protein. In some embodiments, the binding region on the DDB1 protein comprises PHE382 of the DDB1 protein. In some embodiments, the binding region on the DDB1 protein comprises SER720 of the DDB1 protein. In some embodiments, the binding region on the DDB1 protein comprises ARG722 of the DDB1 protein. In some embodiments, the binding region on the DDB1 protein comprises LYS723 of the DDB1 protein. In some embodiments, the binding region on the DDB1 protein comprises SER738 of the DDB1 protein. In some embodiments, the binding region on the DDB1 protein comprises ILE740 of the DDB1 protein. In some embodiments, the binding region on the DDB1 protein comprises GLU787 of the DDB1 protein. In some embodiments, the binding region on the DDB1 protein comprises TYR812 of the DDB1 protein. In some embodiments, the binding region on the DDB1 protein comprises LEU814 of the DDB1 protein. In some embodiments, the binding region on the DDB1 protein comprises SER815 of the DDB1 protein. In some embodiments, the binding region on the DDB1 protein comprises ALA834 of the DDB1 protein. In some embodiments, the binding region on the DDB1 protein comprises VAL836 of the DDB1 protein. In some embodiments, the binding region on the DDB1 protein comprises ALA841 of the DDB1 protein. In some embodiments, the binding region on the DDB1 protein comprises ALA869 of the DDB1 protein. In some embodiments, the binding region on the DDB1 protein comprises TYR871 of the DDB1 protein. In some embodiments, the binding region on the DDB1 protein comprises SER872 of the DDB1 protein. In some embodiments, the binding region on the DDB1 protein comprises MET910 of the DDB1 protein. In some embodiments, the binding region on the DDB1 protein comprises LEU912 of the DDB1 protein. In some embodiments, the binding region on the DDB1 protein comprises TYR913 of the DDB1 protein. In some embodiments, the binding region on the DDB1 protein comprises LEU926 of the DDB1 protein. In some embodiments, the binding region on the DDB1 protein comprises TRP953 of the DDB1 protein. In some embodiments, the binding region on the DDB1 protein comprises SER955 of the DDB1 protein. In some embodiments, the binding region on the DDB1 protein comprises ALA956 of the DDB1 protein. In some embodiments, the binding region on the DDB1 protein comprises ASN970 of the DDB1 protein. In some embodiments, the binding region on the DDB1 protein comprises ALA971 of the DDB1 protein. In some embodiments, the binding region on the DDB1 protein comprises PHE972 of the DDB1 protein. In some embodiments, the binding region on the DDB1 protein comprises PHE1003 of the DDB1 protein. In some embodiments, the binding region on the DDB1 protein comprises ASN1005 of the DDB1 protein. In some embodiments, the binding region on the DDB1 protein comprises VAL1006 of the DDB1 protein. In some embodiments, the binding region on the DDB1 protein comprises VAL1033 of the DDB1 protein.

In some embodiments, the binding between the DDB1 protein and the ligand comprises one or more of a salt-bridge, a Coulombic interaction, a hydrogen bond, a stereoelectronic interaction, and a dispersion contact. In some embodiments, the binding between the DDB1 protein and the ligand comprises a salt-bridge. In some embodiments, the binding between the DDB1 protein and the ligand comprises a Coulombic interaction. In some embodiments, the binding between the DDB1 protein and the ligand comprises one or more hydrogen bonds. In some embodiments, the binding between the DDB1 protein and the ligand comprises a stereoelectronic interaction. In some embodiments, the binding between the DDB1 protein and the ligand comprises dispersion contacts.

In some embodiments, the DDB1 protein comprises a BPC domain comprising a central cavity. In some embodiments, the ligand binds the DDB1 protein in the central cavity of the BPC domain. In some embodiments, the DDB1 protein comprises a WD40-motiff. In some embodiments, the WD40-motiff comprises a center. In some embodiments, the ligand is anchored toward the center of the WD40-motiff. In some embodiments, the ligand is anchored toward the center of the WD40-motiff by a salt-bridge. In some embodiments, the ligand includes a nitro group. In some embodiments, the salt-bridge is between the primary amine of an amino acid of the DDB1 protein and the ligand's nitro group. In some embodiments, the salt-bridge is between the primary amine of a lysine (e.g. LYS723) of the DDB1 protein and the ligand's nitro group.

In some embodiments, the ligand is anchored toward the center of the WD40-motiff by a Coulombic interaction. In some embodiments, the ligand includes an electron deficient nitrogen. In some embodiments, the nitro group includes an electron deficient nitrogen. In some embodiments, the Coulombic interaction is between the electron-deficient nitrogen and a lone-pair of a nearby water. In some embodiments, the nearby water is ordered between a backbone carbonyl oxygen atom of one or more amino acids of the DDB1 protein. In some embodiments, the nearby water is ordered between a backbone carbonyl oxygen atom of an arginine (e.g. ARG722) of the DDB1 protein. In some embodiments, the nearby water is ordered between a backbone carbonyl oxygen atom of a valine (e.g. VAL360) of the DDB1 protein. In some embodiments, the nearby water is ordered between the primary amine of a lysine such as LYS723. In some embodiments, the nearby water is ordered between the backbone carbonyl oxygen atom of the arginine, and the backbone carbonyl oxygen atom of the valine, and/or the primary amine of the lysine. In some embodiments, the nearby water is ordered between the backbone carbonyl oxygen atoms of ARG722 and VAL360 as well as the primary amine of LYS723. In some embodiments, the ligand is anchored toward the center of the WD40-motiff by the Coulombic interaction and the salt-bridge.

In some embodiments, the ligand includes a thiazole. In some embodiments, the ligand includes an amide. In some embodiments, the ligand includes an acetate. In some embodiments, the ligand includes one or more pi-faces. In some embodiments, the ligand includes a pi-face of a thiazole. In some embodiments, the ligand includes a pi-face of an amide. In some embodiments, the pi-faces of the thiazole and the amide rest over an amino acid sidechain. In some embodiments, the pi-faces of the thiazole and the amide rest over a valine (e.g. VAL360) sidechain. In some embodiments, the amide forms an intermolecular hydrogen bond with a sidechain of an amino acid of the DDB1 protein. In some embodiments, the amide forms a hydrogen bond with a sidechain of an asparagine (e.g. ASN1005) of the DDB1 protein. In some embodiments, the amide forms an intramolecular hydrogen bond with the acetate. In some embodiments, the amide forms an intermolecular hydrogen bond with a sidechain of the asparagine and an intramolecular hydrogen bond with the acetate. In some embodiments, the ligand includes thiophene comprising a sulfur. In some embodiments, the sulfur of the thiophene is geometrically stabilized through a stereoelectronic interaction with an amino acid sidechain of the DDB1 protein. In some embodiments, the sulfur of the thiophene is geometrically stabilized through a stereoelectronic interaction with the sidechain of the asparagine (e.g. ASN1005). In some embodiments, the acetate comprises a methyl group that forms a dispersion contact with an ordered water. In some embodiments, the acetate comprises a methyl group that forms a dispersion contact with an amino acid sidechain of the DDB1 protein. In some embodiments, the acetate comprises a methyl group that forms a dispersion contact with an arginine (e.g. ARG722) sidechain of the DDB1 protein. In some embodiments, the acetate comprises a methyl group that forms dispersion contacts with the arginine sidechain of the DDB1 protein and an ordered water. In some embodiments, the ligand includes a benzene ring. In some embodiments, the benzene ring forms dispersion contacts with amino acid sidechains of the DDB1 protein. In some embodiments, the benzene ring forms a dispersion contact with an alanine (e.g. ALA381) sidechain of the DDB1 protein. In some embodiments, the benzene ring forms a dispersion contact with a leucine (e.g. LEU328) sidechain of the DDB1 protein. In some embodiments, the benzene ring forms a dispersion contact with a proline (e.g. PRO358) sidechain of the DDB1 protein. In some embodiments, the benzene ring forms a dispersion contact with a valine (e.g. VAL1033) sidechain of the DDB1 protein. In some embodiments, the benzene ring forms dispersion contacts with the alanine, leucine, proline, and valine sidechains of the DDB1 protein. In some embodiments, the benzene ring forms dispersion contacts with ALA381, LEU328, PRO358 and VAL1033 sidechains of the DDB1 protein.

Disclosed herein, in some embodiments, are in vivo modified proteins. In some embodiments, the binding between the DDB1 protein and the ligand comprises a binding affinity with an equilibrium dissociation constant (Kd) below 100 μM, a Kd below 90 μM, a Kd below 80 μM, a Kd below 70 μM, a Kd below 60 μM, below 50 μM, a Kd below 45 μM, a Kd below 40 μM, a Kd below 35 μM, a Kd below 30 μM, a Kd below 25 μM, a Kd below 20 μM, a Kd below 15 μM, a Kd below 14 μM, a Kd below 13 μM, a Kd below 12 μM, a Kd below 11 μM, a Kd below 10 μM, a Kd below 9 μM, a Kd below 8 μM, a Kd below 7 μM, a Kd below 6 μM, a Kd below 5 μM, a Kd below 4 μM, a Kd below 3 μM, a Kd below 2 μM, or a Kd below 1 μM. In some embodiments, the binding between the DDB1 protein and the ligand comprises a binding affinity with a Kd<20 μM, a Kd from 20-100 μM, or a Kd>100 μM. An in vivo engineered DDB1 protein may include a DDB1 protein bound to a ligand with any of the aforementioned binding affinities.

Disclosed herein, in some embodiments, are in vivo modified proteins. In some embodiments, the binding between the DDB1 binding moiety and the DDB1 protein is non-covalent. The binding may include a non-covalent bond. The binding may include more than one non-covalent bond. Some non-limiting examples of non-covalent bonds include a salt-bridge, a Coulombic interaction, a hydrogen bond, a stereoelectronic interaction, or a dispersion contact. The binding may include a combination of non-covalent bonds. In some embodiments, the binding between the DDB1 binding moiety and the DDB1 protein is covalent.

### Ligand-Protein Complex

Disclosed herein, in some embodiments, are ligand-protein complexes. In some embodiments, the ligand-protein complex comprises a ligand-DNA damage-binding protein 1 (DDB1) complex. In some embodiments, the ligand-DDB1 complex is formed by binding a DDB1 protein to a ligand. In some embodiments, the ligand is a DDB1 ligand. In some embodiments, the binding is directly between the DDB1 protein and the ligand. In some embodiments, the DDB1 protein is directly bound to the ligand. In some embodiments, the binding is non-covalent. In some embodiments, the binding is covalent. In some embodiments, the DDB1 is directly bound to the ligand. In some embodiments, the ligand comprises a compound disclosed herein, or a salt or variant thereof. The ligand may be any ligand described herein. In some embodiments, the ligand comprises a DDB1 binding moiety such as a DDB1 binding moiety described herein. In some embodiments, the DDB1 ligand is a heterobifunctional compound comprising a DDB1 binding moiety covalently connected through a linker to a target protein binding moiety described herein.

Disclosed herein, in some embodiments, are ligand-protein complexes. In some embodiments, the ligand-DDB1 complex is formed by non-covalently binding a DDB1 protein directly to a ligand, the ligand comprising a DDB1 binding moiety. In some embodiments, the ligand-DDB1 complex is formed by covalently binding a DDB1 protein directly to a ligand, the ligand comprising a DDB1 binding moiety. In some embodiments, the ligand-DDB1 complex is formed by non-covalently binding a DDB1 protein directly to a heterobifunctional compound, the heterobifunctional compound comprising a DDB1 binding moiety covalently connected through a linker to a target protein binding moiety. In some embodiments, the ligand-DDB1 complex is formed by covalently binding a DDB1 protein directly to a heterobifunctional compound, the heterobifunctional compound comprising a DDB1 binding moiety covalently connected through a linker to a target protein binding moiety.

Disclosed herein, in some embodiments, are ligand-protein complexes. In some embodiments, the ligand comprises a DDB1 binding moiety. In some embodiments, the ligand comprises a linker. In some embodiments, the ligand comprises a target protein binding moiety. In some embodiments, the DDB1 binding moiety is covalently connected to a linker. In some embodiments, the linker is further connected to a target protein binding moiety. In some embodiments, the DDB1 binding moiety is covalently connected through a linker to a target protein binding moiety. In some embodiments, the DDB1 binding moiety is covalently connected to a target protein binding moiety without a linker. In some embodiments, target protein binding moiety binds to a target protein such as a target protein described herein. In some embodiments, the ligand comprises a compound described herein. For example, the ligand may comprise a DDB1 binding moiety disclosed herein, or the ligand may comprise a linker disclosed herein, or the ligand may comprise a target protein binding moiety disclosed herein. In some embodiments, the ligand is a small molecule. In some embodiments, the ligand is a heterobifunctional compound comprising a DDB1 binding moiety covalently connected through a linker to a target protein binding moiety.

Disclosed herein, in some embodiments, are ligand-protein complexes. In some embodiments, the DDB1 binding moiety is bound to a binding region on the DDB1 protein. In some embodiments, the binding region on the DDB1 protein comprises a beta propeller domain. In some embodiments, the beta propeller domain comprises a beta propeller C (BPC) domain. In some embodiments, the binding region on the DDB1 protein comprises a BPC domain. In some embodiments, the binding region on the DDB1 protein comprises a top face of the BPC domain.

Disclosed herein, in some embodiments, are ligand-protein complexes. In some embodiments, the binding region on the DDB1 protein comprises one or more of the following DDB1 residues: ARG327, LEU328, PRO358, ILE359, VAL360, ASP361, GLY380, ALA381, PHE382, SER720, ARG722, LYS723, SER738, ILE740, GLU787, TYR812, LEU814, SER815, ALA834, VAL836, ALA841, ALA869, TYR871, SER872, MET910, LEU912, TYR913, LEU926, TRP953, SER955, ALA956, ASN970, ALA971, PHE972, PHE1003, ASN1005, VAL1006, or VAL1033. In some embodiments, one or more of the following DDB1 residues are involved in the non-covalent binding between the DDB1 protein and the ligand: ARG327, LEU328, PRO358, ILE359, VAL360, ASP361, GLY380, ALA381, PHE382, SER720, ARG722, LYS723, SER738, ILE740, GLU787, TYR812, LEU814, SER815, ALA834, VAL836, ALA841, ALA869, TYR871, SER872, MET910, LEU912, TYR913, LEU926, TRP953, SER955, ALA956, ASN970, ALA971, PHE972, PHE1003, ASN1005, VAL1006, or VAL1033. In some embodiments, the binding region on the DDB1 protein comprises an amino acid residue described herein, such as in the section titled “Modified Proteins.”

In some embodiments, the binding between the DDB1 protein and the ligand comprises one or more of a salt-bridge, a Coulombic interaction, a hydrogen bond, a stereoelectronic interaction, and a dispersion contact. In some embodiments, the binding between the DDB1 protein and the ligand comprises a salt-bridge. In some embodiments, the binding between the DDB1 protein and the ligand comprises a Coulombic interaction. In some embodiments, the binding between the DDB1 protein and the ligand comprises one or more hydrogen bonds. In some embodiments, the binding between the DDB1 protein and the ligand comprises a stereoelectronic interaction. In some embodiments, the binding between the DDB1 protein and the ligand comprises a dispersion contact.

In some embodiments, the DDB1 protein comprises a BPC domain comprising a central cavity. In some embodiments, the ligand binds the DDB1 protein in the central cavity of the BPC domain. In some embodiments, the DDB1 protein comprises a WD40-motiff. In some embodiments, the WD40-motiff comprises a center. In some embodiments, the ligand is anchored toward the center of the WD40-motiff. In some embodiments, the ligand is anchored toward the center of the WD40-motiff by a salt-bridge. In some embodiments, the ligand includes a nitro group. In some embodiments, the salt-bridge is between the primary amine of an amino acid of the DDB1 protein and the ligand's nitro group. In some embodiments, the salt-bridge is between the primary amine of a lysine (e.g. LYS723) of the DDB1 protein and the ligand's nitro group.

In some embodiments, the ligand is anchored toward the center of the WD40-motiff by a Coulombic interaction. In some embodiments, the ligand includes an electron deficient nitrogen. In some embodiments, the nitro group includes an electron deficient nitrogen. In some embodiments, the Coulombic interaction is between the electron-deficient nitrogen and a lone-pair of a nearby water. In some embodiments, the nearby water is ordered between a backbone carbonyl oxygen atom of one or more amino acids of the DDB1 protein. In some embodiments, the nearby water is ordered between a backbone carbonyl oxygen atom of an arginine (e.g. ARG722) of the DDB1 protein. In some embodiments, the nearby water is ordered between a backbone carbonyl oxygen atom of a valine (e.g. VAL360) of the DDB1 protein. In some embodiments, the nearby water is ordered between the primary amine of a lysine such as LYS723. In some embodiments, the nearby water is ordered between the backbone carbonyl oxygen atom of the arginine, and the backbone carbonyl oxygen atom of the valine, and/or the primary amine of the lysine. In some embodiments, the nearby water is ordered between the backbone carbonyl oxygen atoms of ARG722 and VAL360 as well as the primary amine of LYS723. In some embodiments, the ligand is anchored toward the center of the WD40-motiff by the Coulombic interaction and the salt-bridge.

In some embodiments, the ligand includes a thiazole. In some embodiments, the ligand includes an amide. In some embodiments, the ligand includes an acetate. In some embodiments, the ligand includes one or more pi-faces. In some embodiments, the ligand includes a pi-face of a thiazole. In some embodiments, the ligand includes a pi-face of an amide. In some embodiments, the pi-faces of the thiazole and the amide rest over an amino acid sidechain. In some embodiments, the pi-faces of the thiazole and the amide rest over a valine (e.g. VAL360) sidechain. In some embodiments, the amide forms an intermolecular hydrogen bond with a sidechain of an amino acid of the DDB1 protein. In some embodiments, the amide forms a hydrogen bond with a sidechain of an asparagine (e.g. ASN1005) of the DDB1 protein. In some embodiments, the amide forms an intramolecular hydrogen bond with the acetate. In some embodiments, the amide forms an intermolecular hydrogen bond with a sidechain of the asparagine and an intramolecular hydrogen bond with the acetate. In some embodiments, the ligand includes thiophene comprising a sulfur. In some embodiments, the sulfur of the thiophene is geometrically stabilized through a stereoelectronic interaction with an amino acid sidechain of the DDB1 protein. In some embodiments, the sulfur of the thiophene is geometrically stabilized through a stereoelectronic interaction with the sidechain of the asparagine (e.g. ASN1005). In some embodiments, the acetate comprises a methyl group that forms a dispersion contact with an ordered water. In some embodiments, the acetate comprises a methyl group that forms a dispersion contact with an amino acid sidechain of the DDB1 protein. In some embodiments, the acetate comprises a methyl group that forms a dispersion contact with an arginine (e.g. ARG722) sidechain of the DDB1 protein. In some embodiments, the acetate comprises a methyl group that forms dispersion contacts with the arginine sidechain of the DDB1 protein and an ordered water. In some embodiments, the ligand includes a benzene ring. In some embodiments, the benzene ring forms dispersion contacts with amino acid sidechains of the DDB1 protein. In some embodiments, the benzene ring forms a dispersion contact with an alanine (e.g. ALA381) sidechain of the DDB1 protein. In some embodiments, the benzene ring forms a dispersion contact with a leucine (e.g. LEU328) sidechain of the DDB1 protein. In some embodiments, the benzene ring forms a dispersion contact with a proline (e.g. PRO358) sidechain of the DDB1 protein. In some embodiments, the benzene ring forms a dispersion contact with a valine (e.g. VAL1033) sidechain of the DDB1 protein. In some embodiments, the benzene ring forms dispersion contacts with the alanine, leucine, proline, and valine sidechains of the DDB1 protein. In some embodiments, the benzene ring forms dispersion contacts with ALA381, LEU328, PRO358 and VAL1033 sidechains of the DDB1 protein.

Disclosed herein, in some embodiments, are ligand-protein complexes. In some embodiments, the binding between the DDB1 protein and the ligand comprises a binding affinity with an equilibrium dissociation constant (Kd) below 100 μM, a Kd below 90 μM, a Kd below 80 μM, a Kd below 70 PM, a Kd below 60 μM, a Kd below 50 μM, a Kd below 45 μM, a Kd below 40 μM, a Kd below 35 μM, a Kd below 30 μM, a Kd below 25 μM, a Kd below 20 μM, a Kd below 15 μM, a Kd below 14 μM, a Kd below 13 μM, a Kd below 12 μM, a Kd below 11 μM, a Kd below 10 μM, a Kd below 9 μM, a Kd below 8 μM, a Kd below 7 μM, a Kd below 6 μM, a Kd below 5 μM, a Kd below 4 μM, a Kd below 3 μM, a Kd below 2 μM, or a Kd below 1 μM. In some embodiments, the binding between the DDB1 protein and the ligand comprises a binding affinity with a Kd<20 μM, a Kd from 20-100 μM, or a Kd>100 μM.

Disclosed herein, in some embodiments, are ligand-protein complexes. In some embodiments, the binding between the DDB1 binding moiety and the DDB1 protein is non-covalent. In some embodiments, the binding between the DDB1 binding moiety and the DDB1 protein is covalent.

Disclosed herein, in some embodiments, are ligand-protein complexes. In some embodiments, the complex is formed in vivo. In some embodiments, the complex is formed in vitro.

### IV. Methods of Treatment and Pharmaceutical Compositions

Disclosed herein, in some embodiments, are heterobifunctional compounds (for example, compounds of Formula (I), or a pharmaceutically acceptable salt or solvate thereof) for use in a method such as a method of treatment. Some embodiments include a heterobifunctional compound for use in a method of degrading, inhibiting, or modulating a protein or a target protein (e.g. a cyclin or a cyclin dependent kinase). Some embodiments include a heterobifunctional compound for use in a method of treating a disease or disorder, in particular cancer, mediated by a target protein (e.g. a cyclin or a cyclin dependent kinase (CDK)).

In certain embodiments, the compounds described herein are used to treat a subject. In certain embodiments, the compounds described herein are used to degrade a target protein. Some embodiments include administering a compound described herein to a subject. Some embodiments include administering a pharmaceutical composition comprising a heterobifunctional compound described herein to a subject. Some embodiments include providing a heterobifunctional compound or pharmaceutical composition described herein for administration to a subject.

In one aspect, provided herein is a method for the treatment of abnormal cell growth (e.g., cancer), in a subject in need thereof, comprising administering to the subject a therapeutically effective amount of a heterobifunctional compound as described herein, or a pharmaceutically acceptable salt thereof. The heterobifunctional compound may be administered as a single agent, or in combination with other therapeutic agents, in particular standard of care agents appropriate for the disease or disorder.

In another aspect, provided herein is a heterobifunctional compound as described herein, or a pharmaceutically acceptable salt thereof, for use in the treatment of abnormal cell growth (e.g., cancer). In another aspect, provided herein is the use of a heterobifunctional compound as described herein, or a pharmaceutically acceptable salt thereof, for the treatment of abnormal cell growth (e.g., cancer). In another aspect, provided herein is a heterobifunctional compound as described herein, or a pharmaceutically acceptable salt thereof, for use in the manufacture of a medicament for treatment of abnormal cell growth (e.g., cancer).

In another aspect, provided herein is a method for the treatment of a disorder mediated by cyclin D, in particular cancer, in a subject in need thereof, comprising administering to the subject a therapeutically effective amount of a heterobifunctional compound as described herein, or a pharmaceutically acceptable salt thereof.

In some embodiments, provided herein is a method for the treatment of cancer in a subject in need thereof, comprising administering to the subject a therapeutically effective amount of the heterobifunctional compound as described herein, or a pharmaceutically acceptable salt thereof.

In some embodiments of each of the methods and uses herein, the cancer is selected from the group consisting of breast cancer, ovarian cancer, bladder cancer, endometrial cancer, uterine cancer, prostate cancer, lung cancer (including NSCLC, SCLC, squamous cell carcinoma or adenocarcinoma), esophageal cancer, head and neck cancer, colorectal cancer, kidney cancer (including RCC), liver cancer (including HCC), pancreatic cancer, stomach (i.e., gastric) cancer, thyroid cancer, and melanoma.

In some embodiments, the method for the treatment comprises administering an effective amount of a heterobifunctional compound of Formula (I) to a subject in need thereof, wherein the target protein binding moiety binds to a CDK, preferably CDK4 and/or CDK6. In some such embodiments, the heterobifunctional compound comprises the structure of Formula (A), (A1), (A2), (A3) or (A4). In preferred embodiments, the heterobifunctional compound comprises the structure of Formula (A-67), (A-70), (A-71) or (A72).

In some embodiments of each of the methods and uses herein, the cancer is cancer is a cyclin D mediated cancer. In some such embodiments, the cancer is characterized by amplification or overexpression of cyclin D (CCND), CDK4, and/or CDK6. In some such embodiments, the cancer is characterized by amplification or overexpression of cyclin D (CCND). In some embodiments, the cancer is characterized by amplification or overexpression of CDK4. In some embodiments, the cancer is characterized by amplification or overexpression of CDK6. In some embodiments, the cancer is characterized by amplification or overexpression of both CCND and CDK4.

In some embodiments of each of the methods and uses herein, the cancer is characterized by primary or acquired resistance to treatment with a CDK4 and/or CDK6 inhibitor, or to endocrine therapy. In some embodiments, the cancer is breast cancer demonstrating such primary or acquired resistance. In some such embodiments, the breast cancer is advanced or metastatic breast cancer. In some embodiments, the breast cancer is hormone receptor positive (HR+), HER2-negative breast cancer. In some embodiments, the breast cancer is HR+, HER2-negative advanced or metastatic breast cancer. In some such embodiments, the breast cancer is triple negative breast cancer (TNBC). In some embodiments, the subject's cancer has progressed on prior treatment with CDK4/6 inhibitors and/or endocrine therapy. In some embodiments, the subject's cancer demonstrates primary or acquired resistance to treatment with CDK4/6 inhibitors and/or endocrine therapy.

In some embodiments, of the methods and uses herein, the heterobifunctional compound is administered as first line therapy. In other embodiments, the heterobifunctional compound is administered as second (or later) line therapy. In some embodiments, the heterobifunctional compound is administered as second (or later) line therapy following treatment with an endocrine therapeutic agent and/or a CDK4/6 inhibitor. In some embodiments, the heterobifunctional compound is administered as second (or later) line therapy following treatment with an endocrine therapeutic agent, e.g., an aromatase inhibitor, a SERM or a SERD. In some embodiments, the heterobifunctional compound is administered as second (or later) line therapy following treatment with a CDK4/6 inhibitor. In some embodiments, the heterobifunctional compound is administered as second (or later) line therapy following treatment with one or more chemotherapy regimens, e.g., including taxanes or platinum agents.

An effective dosage can be administered in one or more administrations. For the purposes of this invention, an effective dosage of drug, compound, or pharmaceutical composition is an amount sufficient to accomplish prophylactic or therapeutic treatment either directly or indirectly. As is understood in the clinical context, an effective dosage of drug, compound or pharmaceutical composition may or may not be achieved in conjunction with another drug, compound or pharmaceutical composition.

In frequent embodiments of the compounds, compositions, methods and uses herein, the methods and uses provide result in one or more of the following effects: (1) inhibiting cancer cell proliferation; (2) inhibiting cancer cell invasiveness; (3) inducing apoptosis of cancer cells; (4) inhibiting cancer cell metastasis; or (5) inhibiting angiogenesis.

In some embodiments, a modified protein disclosed herein is formed in vivo upon administration of the heterobifunctional compound or pharmaceutical composition to the subject. In some embodiments, a ligand-protein complex is formed by administration of the heterobifunctional compound or pharmaceutical composition to the subject.

In certain embodiments, the heterobifunctional compound as described herein is administered as a pure chemical. In other embodiments, the heterobifunctional compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)). One embodiment provides a pharmaceutical composition comprising a compound described herein, or a pharmaceutically acceptable salt thereof, and a pharmaceutically acceptable excipient.

Provided herein is a pharmaceutical composition comprising at least one heterobifunctional compound described herein, or a stereoisomer, pharmaceutically acceptable salt, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition. In some embodiments, the excipient comprises a buffer or solution.

In certain embodiments, a heterobifunctional compound described herein is substantially pure, in that it contains less than about 5%, preferably less than about 1%, or more preferably less than about 0.1% of other organic small molecules, such as unreacted intermediates or synthesis by-products that are created, for example, in one or more of the steps of a synthesis method.

Some embodiments include use of a compound such as a ligand described herein, use of a ligand-DDB1 complex, or use of an in vivo modified DDB1 protein. The use may include a use as an antiviral drug. The use may include a use as a molecule glue. The use may include a use as a targeted protein degrader. In some embodiments, the use comprises administration of the compound to a subject. In some embodiments, the use comprises contact of a sample with the compound.

Provided herein, in some embodiments, is a method for degrading a target protein in a subject. Some embodiments include administering, to the subject, a ligand described herein. Some embodiments include administering, to the subject, a ligand comprising a DNA damage-binding protein 1 (DDB1) binding moiety covalently connected through a linker to a target protein binding moiety. In some embodiments, the subject is a subject in need of administration of the ligand or is in need of treatment with the ligand. Some embodiments include a method of modulating a target protein, comprising administering a therapeutically effective amount of a compound described herein (e.g., a heterobifunctional compound), to a subject in need thereof. In some embodiments, the target protein is decreased in the subject, relative to a baseline measurement. Following administration of a heterobifunctional compound described herein to a subject, a target protein measurement may be decreased in a tissue sample or fluid sample from the subject, relative to a baseline target protein measurement in a first tissue sample or fluid sample from the subject. Some embodiments include measuring a decrease in the CDK following the administration.

Some embodiments include a method of activating apoptosis, comprising administering a therapeutically effective amount of a compound described herein (e.g., a heterobifunctional compound), to a subject in need thereof. Some embodiments include activating a caspase such as caspase 3.

Some embodiments include obtaining a baseline measurement of a target protein. The baseline measurement may be obtained in a first sample obtained prior to administration of a compound described herein to a subject. The first sample may comprise a fluid sample. The first sample may comprise a tissue sample. The baseline measurement may be obtained directly in the subject. The baseline measurement may include a concentration. The baseline measurement may be normalized, for example to a sample weight, to a sample volume, to a total sample protein measurement, or to a housekeeping protein measurement.

Some embodiments include obtaining a measurement of a target protein. The measurement may be obtained in a second sample obtained after to administration of a compound described herein to a subject. The measurement may be obtained in a second sample obtained during to administration of a compound described herein to a subject. The second sample may comprise a fluid sample. The second sample may comprise a tissue sample. The measurement may be obtained directly in the subject. The measurement may be normalized, for example to a sample weight, to a sample volume, to a total sample protein measurement, or to a housekeeping protein measurement.

Measurements or baseline measurements of target proteins may include any method known in the art. For example, a measurement or baseline measurements may be obtained using an assay such as an immunoassay, a colorimetric assay, a lateral flow assay, a fluorescence assay, a proteomics assay, or a cell-based assay. The immunoassay may include an immunoblot such as a western blot or a dot blot, an enzyme-linked immunosorbent assay, or immunostaining. The proteomics assay may include mass spectrometry. A measurement or baseline measurements may be obtained using flow cytometry. A measurement or baseline measurements may be obtained using chromatography, for example high performance liquid chromatography.

The target protein may be or include any target protein included herein, as well as other target proteins not named. Some embodiments include a method of degrading a cyclin dependent kinase (CDK). Some embodiments include a method of degrading a target protein comprising a CDK. Some examples of such cyclin dependent kinases include, but are not limited to, CDK4 or CDK6. Some embodiments include a method of modulating a CDK, comprising administering a therapeutically effective amount of a compound described herein (e.g., a heterobifunctional compound), to a subject in need thereof. In some embodiments, the CDK is decreased in the subject, relative to a baseline measurement. Some embodiments include measuring a decrease in the CDK following the administration.

Some embodiments include a method of degrading a cyclin. Some embodiments include a method of degrading a target protein comprising a cyclin. Some examples of such cyclins include a cyclin D such as cyclin D1, or cyclin D2, cyclin D3, or cyclin E. Some embodiments include a method of modulating a cyclin, comprising administering a therapeutically effective amount of a compound described herein (e.g., a heterobifunctional compound), to a subject in need thereof. Some embodiments include a method of modulating Cyclin D, comprising administering a therapeutically effective amount of a compound described herein (e.g., a heterobifunctional compound), to a subject in need thereof. In some embodiments, the cyclin is decreased in the subject, relative to a baseline measurement. Some embodiments include measuring a decrease in the cyclin following the administration.

Some embodiments include a method of degrading a transcription factor. Non-limiting examples of transcription factors include CBP and P300. Some embodiments include a method of degrading a target protein comprising CBP or P300. Some embodiments include a method of degrading a target protein comprising CBP. Some embodiments include a method of degrading a target protein comprising P300. Some embodiments include a method of modulating a transcription factor, comprising administering a therapeutically effective amount of a compound described herein (e.g., a heterobifunctional compound), to a subject in need thereof. In some embodiments, the transcription factor is decreased in the subject, relative to a baseline measurement. Some embodiments include measuring a decrease in the transcription factor following the administration. Additional examples of target proteins are included herein.

Examples of subjects include vertebrates, animals, mammals, dogs, cats, cattle, rodents, mice, rats, primates, monkeys, and humans. In some embodiments, the subject is a mammal. In some embodiments, the subject is a human.

In some embodiments, administering the ligand to the subject comprises administering an effective amount of the ligand sufficient to degrade the target protein. In some embodiments, upon administration of the ligand to the subject, the target protein is ubiquitinated to form a ubiquitinated target protein. In some embodiments, the administration is intravenous. In some embodiments, the administration comprises an injection. In some embodiments, the administration comprises cutaneous administration. In some embodiments, the administration comprises subcutaneous administration. In some embodiments, the administration comprises intraperitoneal administration. In some embodiments, the administration comprises oral administration. In some embodiments, the route of administration is intravenous, oral, subcutaneous, intraperitoneal, ocular, intraocular, intramuscular, interstitial, intraarterial, intracranial, intraventricular, intrasynovial, transepithelial, transdermal, by inhalation, ophthalmic, sublingual, buccal, topical, dermal, rectal, nasal, by insufflation, or by nebulization. In some embodiments, the administration is intramuscular. In some embodiments, the administration is intrathecal. In some embodiments, the administration is subcutaneous. In some embodiments, the administration is oral. In some embodiments, the administration is sublingual. In some embodiments, the administration is buccal. In some embodiments, the administration is rectal. In some embodiments, the administration is vaginal. In some embodiments, the administration is ocular. In some embodiments, the administration is otic. In some embodiments, the administration is nasal. In some embodiments, the administration is inhalation. In some embodiments, the administration is nebulization. In some embodiments, the administration is cutaneous. In some embodiments, the administration is topical. In some embodiments, the administration is transdermal. In some embodiments, the administration is systemic.

Provided herein, in some embodiments, is a method for degrading a target protein in a sample. Some embodiments include contacting a target protein with a ligand described herein. Some embodiments include contacting a target protein with a ligand comprising a DNA damage-binding protein 1 (DDB1) binding moiety covalently connected through a linker to a target protein binding moiety.

In some embodiments, the sample is a biological sample. In some embodiments, the biological sample comprises a tissue, a cell, or a biological fluid. In some embodiments, the contact is in vitro. In some embodiments, the contact is in vivo. In some embodiments, upon being contacted with the ligand, the target protein is ubiquitinated to form a ubiquitinated target protein.

In some embodiments, upon administration or contact, the ubiquitinated target protein is degraded. In some embodiments, the ubiquitinated target protein is degraded. In some embodiments, the degradation of the target protein is specific to the target protein. In some embodiments, the target protein comprises proteasomal degradation. In some embodiments, the target protein is degraded by a proteasome.

In some embodiments, upon administration or contact, the ligand binds to a DDB1 protein to form a ligand-DDB1 complex. In some embodiments, the ligand directly binds to the DDB1 protein through the DDB1 binding moiety of the ligand. In some embodiments, the binding between the DDB1 binding moiety and the DDB1 protein is non-covalent. In some embodiments, the binding between the DDB1 binding moiety and the DDB1 protein is covalent. In some embodiments, the target protein is ubiquitinated by a ubiquitin E3 ligase complex comprising the DDB1 protein. In some embodiments, the ligand (e.g. a DDB1 ligand) recruits the ubiquitin E3 ligase complex to the target protein via the DDB1 binding moiety. In some embodiments, the ligand is a small molecule. In some embodiments, the ligand comprises a targeted protein degrader. In some embodiments, the ligand is synthetic. In some embodiments, the ligand comprises a ligand described herein.

The target protein to degraded using a method described herein may be or include any target protein described herein. In some embodiments, the target protein comprises any one of a transcription factor, CBP, p300, a kinase, a receptor, a TRK, TrkA, TrkB, TrkC, a cyclin dependent kinase, CDK4, CDK6, B7.1, B7, TINFRlm, TNFR2, NADPH oxidase, a partner in an apoptosis pathway, BclIBax, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide synthase, cyclo-oxygenase 1, cyclo-oxygenase 2, a receptor, a 5HT receptor, a dopamine receptor, a G-protein, Gq, a histamine receptor, 5-lipoxygenase, tryptase serine protease, thymidylate synthase, purine nucleoside phosphorylase, GAPDH, a trypanosomal protein, glycogen phosphorylase, carbonic anhydrase, a chemokine receptor, JAK, STAT, RXR, RAR, HIV 1 protease, HIV 1 integrase, influenza, neuramimidase, hepatitis B reverse transcriptase, sodium channel, multi drug resistance, protein P-glycoprotein, MRP, a tyrosine kinase, CD23, CD124, tyrosine kinase p56 lck, CD4, CD5, IL-2 receptor, IL-1 receptor, TNF-alphaR, ICAM1, a Ca+ channel, VCAM, an integrin, a VLA-4 integrin, a selectin, CD40, CD40L, a neurokinin, a neurokinin receptor, inosine monophosphate dehydrogenase, p38 MAP Kinase, Ras, Raf, Mek, Erk, interleukin-1 converting enzyme, a caspase, HCV, NS3 protease, HCV NS3 RNA helicase, glycinamide ribonucleotide formyl transferase, rhinovirus 3C protease, herpes simplex virus-1, a protease, cytomegalovirus protease, poly ADP-ribose polymerase, vascular endothelial growth factor, oxytocin receptor, microsomal transfer protein inhibitor, bile acid transport inhibitor, a 5 alpha reductase inhibitor, angiotensin II, a glycine receptor, a noradrenaline reuptake receptor, an endothelin receptor, neuropeptide Y, a neuropeptide Y receptor, an estrogen receptor, an androgen receptor, an adenosine receptor, an adenosine kinase, AMP deaminase, a purinergic receptor, P2Y1, P2Y2, P2Y4, P2Y6, P2X1-7, a farnesyltransferase, geranylgeranyl transferase, an NGF receptor, beta-amyloid, tyrosine kinase Flk-IIKDR, vitronectin receptor, an integrin receptor, Her2 neu, telomerase inhibition, cytosolic phospholipaseA2, EGF receptor tyrosine kinase, ecdysone 20-monooxygenase, ion channel of the GABA gated chloride channel, acetylcholinesterase, voltage-sensitive sodium channel protein, calcium release channel, a chloride channel, acetyl-CoA carboxylase, adenylosuccinate synthetase, protoporphyrinogen oxidase, or enolpyruvylshikimate-phosphate synthase. Some embodiments include multiple target proteins, such as a combination of any two or more of the target proteins disclosed herein.

A heterobifunctional compound (such as a compound comprising a DDB1 binding moiety) described herein may be useful for several purposes, including but not limited to use: 1) as an antiviral drug; 2) as a DDB1 protein level modulator (e.g. increasing or decreasing DDB1 protein levels); 3) as a DDB1 function modulator (e.g. activating or inhibiting DDB1); 4) as a molecular glue (e.g. increasing a protein-protein interaction between DDB1 and a second protein, such as a target protein); 5) for affecting activity or protein levels of the second protein via the molecule glue function (e.g., by acting as a targeted protein degrader); 6) for decreasing protein levels of the second protein via the molecule glue function; 7) for increasing protein levels of the second protein via the molecule glue function; 8) for decreasing activity of the second protein via the molecule glue function; or 9) for increasing activity of the second protein via the molecule glue function.

In some embodiments, the heterobifunctional compounds described herein may compete for binding to DDB1 with one or more viral proteins or viral-derived peptides. In some embodiments, the heterobifunctional compound competitively binds to the same binding site on DBB1 as a viral protein or a viral-derived peptide. Such competitive binding can be measured with a competition binding assay and used to identify and characterize the residues comprising the DBB1 binding site of the hetero-bifunctional compound.

A heterobifunctional compound described herein may be useful for treating a disease or disorder. For example, the compound may be administered to a subject having the disease or disorder. The administration may reduce the severity of the disease or disorder in the subject, relative to a baseline measurement. The compound may bind a target protein involved in the disease or disorder, resulting in inhibition or degradation of the target protein. The compound may be a heterobifunctional compound and comprise a DDB1 binding moiety and a target protein binding moiety, wherein the target protein is involved in the disease or disorder. The target protein may exacerbate the disease or disorder. The target protein may prevent or decrease inhibition of the disease or disorder.

In some embodiments, a compound described herein is used as an antimicrobial drug. For example, the compound may be administered to a subject having a microbial infection. The administration may reduce the severity of the microbial infection in the subject, relative to a baseline measurement. The compound may bind a target protein involved in the microbial infection, resulting in inhibition or degradation of the target protein. The microbial infection may include a virus infection. The microbial infection may include a bacterial infection. The compound may be a heterobifunctional compound and comprise a DDB1 binding moiety and a target protein binding moiety, wherein the target protein is a microbial protein. The microbial protein may include a viral protein. The microbial protein may include a bacterial protein. The target protein may be a non-microbial protein that exacerbates the microbial infection. The target protein may be a non-microbial protein that prevents or decreases inhibition of the microbial infection. In some embodiments, the compound enters a cell of the subject, binds to a microbial protein in the cell via its target protein binding moiety, binds DDB1 via its DDB1 binding moiety, and induces ubiquitin-mediated degradation of the microbial protein. Such an action may be useful against microbes such as bacteria or viruses that infect or reside within the cell.

A compound described herein may be useful for modulating DDB1 protein levels. For example, the compound may be used to increase or decrease DDB1 protein levels. In some embodiments, a compound comprising a DDB1 binding moiety described herein, is used to increase DDB1 protein levels. For example, the compound may bind to DDB1 and prevent its degradation. In some embodiments, a compound comprising a DDB1 binding moiety described herein, is used to decrease DDB1 protein levels. For example, the compound may bind to DDB1 and increase its degradation. The compound may be a heterobifunctional compound and include a DDB1 binding moiety coupled to (directly or through a linker) a second moiety that increases degradation of the DDB1 protein, or that decreases degradation of the DDB1 protein. The second moiety may accomplish this by binding to a target protein. In some such embodiments, the target protein may include an E3 ubiquitin ligase protein that enhances degradation of the DDB1 protein. In some embodiments, the heterobifunctional compound comprises or consists of a DDB1 binding moiety. In some embodiments, the heterobifunctional compound comprises or consists of the structure of Formula (I), or a pharmaceutically acceptable salt or solvate thereof, a compound provided in Table 4, or pharmaceutically acceptable salt thereof. In some embodiments, the heterobifunctional compound is administered to a subject to increase a DDB1 protein level in the subject. The administration may increase DDB1 activity in the subject, relative to a baseline measurement. In some embodiments, the compound is administered to a subject to decrease a DDB1 protein level in the subject. The administration may decrease DDB1 activity in the subject, relative to a baseline measurement.

A heterobifunctional compound described herein may be useful for modulating DDB1 function. For example, the compound may be used to activate or inhibit DDB1. In some embodiments, a compound comprising a DDB1 binding moiety described herein, is used to increase DDB1 activity. For example, the compound may bind to DDB1 and activate DDB1. The compound may allosterically activate DDB1. The compound may activate DDB1 by binding to a protein binding site on DDB1. In some embodiments, a heterobifunctional compound comprising a DDB1 binding moiety described herein, is used to decrease DDB1 activity. For example, the compound may bind to DDB1 and inhibit DDB1. The compound may allosterically inhibit DDB1. The compound may inhibit DDB1 by binding to an active site of DDB1. The compound may inhibit DDB1 by binding to a protein binding site on DDB1. The compound may be a heterobifunctional compound and include a DDB1 binding moiety coupled to (directly or through a linker) a second moiety that increases activity of the DDB1 protein, or that decreases activity of the DDB1 protein. The second moiety may accomplish this by binding to a target protein. In some embodiments, the compound is administered to a subject to increase DDB1 activity in the subject. The administration may increase DDB1 activity in the subject, relative to a baseline measurement. In some embodiments, the compound is administered to a subject to decrease DDB1 activity in the subject. The administration may decrease DDB1 activity in the subject, relative to a baseline measurement.

A heterobifunctional compound described herein may be useful as a molecular glue. For example, the compound may bind multiple molecules and hold them together. In some embodiments, the molecular glue binds DDB1 and a target protein. The compound may accomplish this as a heterobifunctional compound that comprises a DDB1 binding moiety and a target protein binding moiety. The compound may increase a protein-protein interaction between DDB1 and a target protein. The compound may act as a molecular glue to modulate an activity or amount of the target protein. As a molecular glue, the compound may decrease an amount of the target protein. As a molecular glue, the compound may increase an amount of the target protein. As a molecular glue, the compound may decrease activity of the target protein. As a molecular glue, the compound may increase activity of the target protein.

Disclosed herein, in some embodiments, are methods for degrading a target protein in a cell. The method may include degrading the target protein through direct binding of an intermediate protein (e.g. a first protein) that interacts with the target protein. This may be referred to as bridged degradation. Some embodiments include administering a binding molecule to the cell. The binding molecule may include a ligand or compound disclosed herein. The ligand may be a heterobifunctional compound. The binding molecule may bind a first protein that interacts with the target protein. The target protein may be degraded before the first protein. In some embodiments, the first protein is not degraded. Some embodiments include administering, to the cell, a binding molecule that binds a first protein that interacts with the target protein, thereby degrading target protein, wherein the target protein is degraded before the first protein or wherein the first protein is not degraded. Some embodiments include measuring the target protein in the cell. Some embodiments include measuring the first protein in the cell. In some embodiments, the interaction between the target protein and the first protein is binding. In some embodiments, the interaction between the target protein and the first protein is dimerization. The target protein may include a target protein described herein. The first protein may include another target protein described herein. In some embodiments, the target protein comprises a cyclin. In some embodiments, the target protein comprises Cyclin D. In some embodiments, the Cyclin D comprises Cyclin D1, Cyclin D2, or Cyclin D3. The cyclin D may include Cyclin D1. The cyclin D may include Cyclin D2. The cyclin D may include Cyclin D3. In some embodiments, the first protein comprises a cyclin-dependent kinase (CDK). The CDK may include CDK4. The CDK may include CDK6. In some embodiments, the first protein comprises CDK4 or CDK6. In some embodiments, the binding molecule reduces viability of the cell. In some embodiments, the cell is a eukaryotic cell. In some embodiments, the cell is a mammalian cell. In some embodiments, the cell is a human cell. In some embodiments, the cell is a cancer cell. In some embodiments, administering the binding molecule to the cell comprises administering the binding molecule to a subject comprising the cell. In some embodiments, the binding molecule recruits a ubiquitin E3 ligase that ubiquitinates the target protein. In some embodiments, the E3 ubiquitin ligase comprises DNA damage-binding protein 1 (DDB1). In some embodiments, the binding molecule comprises a heterobifunctional compound comprising an E3 ubiquitin ligase-binding moiety covalently connected through a linker to a first protein binding moiety. The first protein binding moiety may include a target protein binding moiety disclosed herein. In some embodiments, the binding molecule comprises a structure disclosed herein.

Disclosed herein, in some embodiments, are methods (e.g. a bridged degradation method) comprising administering to a cell a binding molecule that binds a cyclin-dependent kinase (CDK), thereby degrading a cyclin that interacts with the CDK. In some embodiments, the cyclin is degraded before the CDK, or wherein the CDK is not degraded. In some embodiments, the cyclin is degraded before the CDK. In some embodiments, the CDK is not degraded.

In some embodiments, the compound of Formula (I) selectively degrades cyclin D relative to CDK4. In some such embodiments, CDK4 is degraded more slowly than cyclin D. In some such embodiments, CDK4 is degraded to a lesser extent than cyclin D. In some embodiments, the compound of Formula (I) degrades cyclin D while CDK4 is not degraded.

Some embodiments include measuring the cyclin in the cell. Some embodiments include measuring the CDK in the cell. In some embodiments, the interaction between the cyclin and the CDK comprises binding or dimerization. The interaction may include binding. The interaction may include dimerization. In some embodiments, the cyclin comprises Cyclin D. In some embodiments, the Cyclin D comprises Cyclin D1, Cyclin D2, or Cyclin D3. The cyclin D may include Cyclin D1. The cyclin D may include Cyclin D2. The cyclin D may include Cyclin D3. In some embodiments, the CDK comprises CDK4 or CDK6. The CDK may include CDK4. The CDK may include CDK6. In some embodiments, the binding molecule reduces viability of the cell. In some embodiments, the cell is a eukaryotic cell. In some embodiments, the cell is a mammalian cell. In some embodiments, the cell is a human cell. In some embodiments, the cell is a cancer cell. In some embodiments, administering the binding molecule to the cell comprises administering the binding molecule to a subject comprising the cell. In some embodiments, the binding molecule recruits a ubiquitin E3 ligase that ubiquitinates the cyclin. In some embodiments, the E3 ubiquitin ligase comprises DNA damage-binding protein 1 (DDB1) In some embodiments, the binding molecule comprises a heterobifunctional compound comprising an E3 ubiquitin ligase-binding moiety covalently connected through a linker to a CDK binding moiety. In some embodiments, the E3 ubiquitin ligase-binding moiety comprises a chemical structure disclosed herein. In some embodiments, the CDK binding moiety comprises a target protein binding moiety disclosed herein. In some embodiments, the binding molecule comprises a ligand disclosed herein.

### Preparation of Compounds

The compounds used in the chemical reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Alternatively, specific and analogous reactants can be identified through the indices of known chemicals and reactions prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compound described herein is P. H. Stahl & C. G. Wermuth “Handbook of Pharmaceutical Salts”, Verlag Helvetica Chimica Acta, Zurich, 2002. The compounds described herein are prepared using the general methods in the art of organic synthesis, as described in the Examples section. Alternative synthetic methods are also used to generate the compounds described herein. Some embodiments include a method of making a heterobifunctional compound disclosed herein.

### Characterization of Examples of Heterobifunctional Compounds

Disclosed herein are heterobifunctional compounds that modulate the protein level of either cyclin D, P300/CBP, or BRD4. These compounds were designed and synthesized by incorporating three moieties: DDB1 ligands, linkers and CDK4/6, P300/CBP, or BRD4 binders.

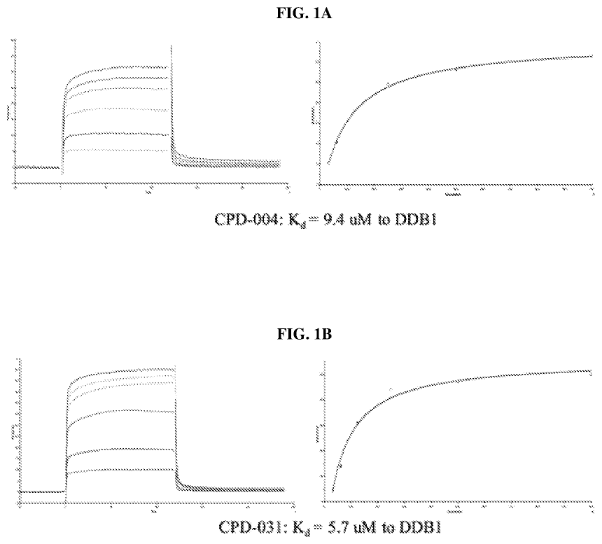

To determine whether the addition of linkers and target binders to the DDB1 ligands affected the binding to DDB1 E3 ligase, the binding affinities of heterobifunctional compounds to DDB1 was evaluated using a surface plasmon resonance (SPR) assay. Purified DDB1ΔBPB proteins were immobilized on a CM5 sensor chip and a dose range of compound solutions were injected in multi-cycle kinetic format. Data was fit to steady state model and gave equivalent dissociation constants (Kd). As illustrated in FIG. 1A-1B, exemplary heterobifunctional compounds, CPD-004 and CPD-031, bound to DDB1 in a concentration-dependent manner, and their binding affinities (Kd) were 9.4 μM and 5.7 μM, respectively (FIG. 1A-1B). Additional exemplary heterobifunctional compounds showed binding affinities (Kd) less than 20 μM, as illustrated in Table 5.

Specific exemplary heterobifunctional compounds were characterized in Calu-1, BT-549 and other cells. Cells that express cyclin D1-3 and CDK4/6 proteins were treated with heterobifunctional compounds disclosed herein at indicated concentrations for 16 hours. Cells were collected, lysed and subject to immunoblotting using an antibody specific to cyclin D1, cyclin D2, cyclin D3, CDK4, CDK6 or phosphorylated R^{b} proteins. Tubulin or GAPDH was used as the loading control. DMSO treatment was used as the negative control. As illustrated in Tables 6A and 6B, following a 16-hour treatment of various heterobifunctional compounds at indicated concentrations, cyclin D1, and CDK4 protein levels in Calu-1 cells were significantly decreased.

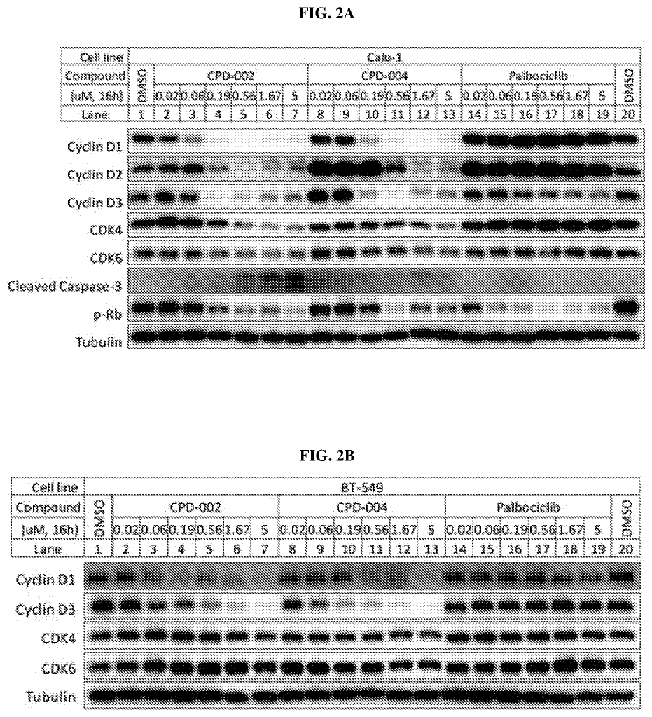

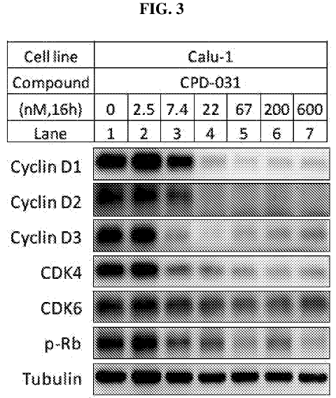

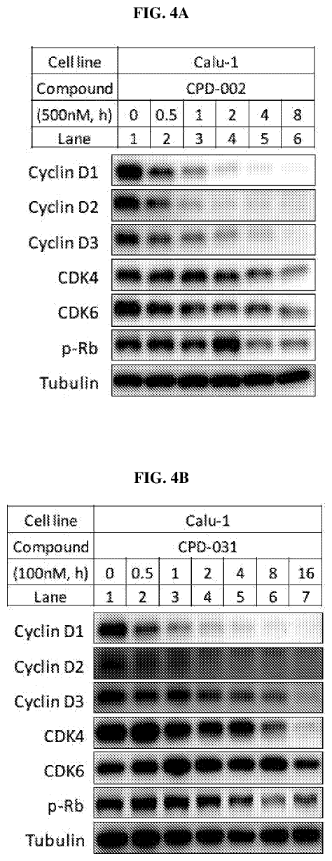

Heterobifunctional compounds, exemplified by CPD-002, CPD-004, and CPD-031, were found to be particularly effective in reducing cyclin D1, cyclin D2, and cyclin D3 protein levels in a concentration-dependent manner (FIG. 2A-2B and FIG. 3; DC₅₀<50 nM for CPD-002, DC₅₀<20 nM for CPD-031). Palbociclib, a CDK4/6 inhibitor, didn't have significant effect on cyclin D and CDK4/6 protein levels (FIG. 2A-2B). Heterobifunctional compounds also inhibited downstream Rb phosphorylation and induced cleaved caspase-3 (cell apoptosis marker) in a concentration-dependent manner in Calu-1 cells (FIG. 2A-2B). In a time-course study, Calu-1 cells were treated with 500 nM CPD-002, or 100 nM CPD-031 for indicated period of time prior to immunoblotting (FIG. 4A-4B). Significant degradation of cyclin D proteins was observed within 0.5 hour, and complete protein degradation was achieved within 2 hours post treatment of CPD-002, while degradation of CDK4 and CDK6 occurred much slower (FIG. 4A). Interestingly, CPD-031 showed slower cyclin D3 degradation compared to cyclin D1 and D2 degradation (FIG. 4B).

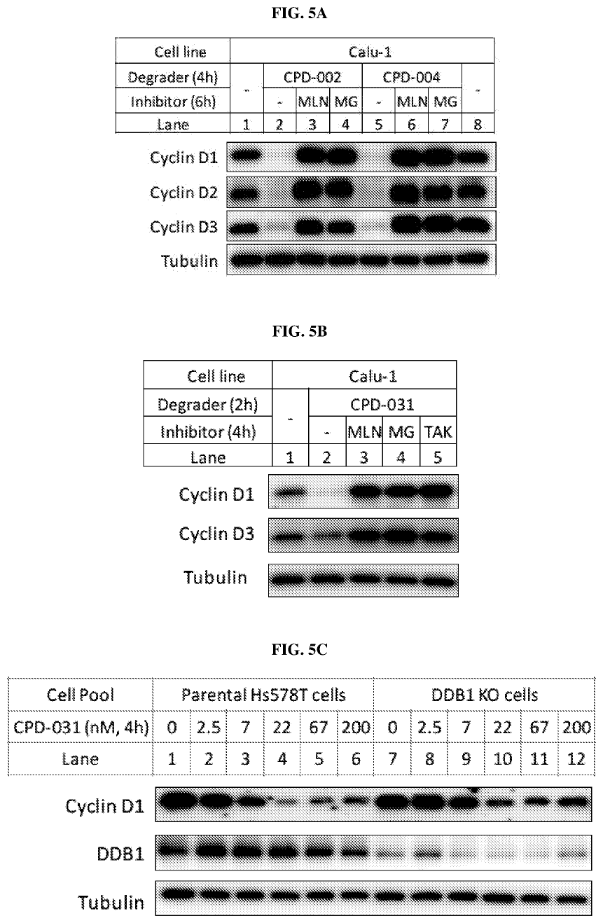

The heterobifunctional compound-mediated degradation was dependent on the ubiquitin-proteasome system and cullin E3 ligase. Pre-treatment of Calu-1 cells with a proteasome inhibitor MG-132, a cullin E3 ligase inhibitor MLN4924, or a ubiquitin activating enzyme (UAE) inhibitor TAK-243, totally diminished cyclin D downregulation effect of CPD-002 or CPD-031 (FIG. 5A-5B). In addition, DDB1 E3 ligase was critical for the degrader-induced cyclin D downregulation. Depletion of DDB1 using CRISPR-Cas9 technology attenuated the cyclin D degradation induced by CPD-031 (FIG. 5C). Taken together, these findings demonstrated that these heterobifunctional compounds downregulated cyclin D proteins via a mechanism mediated by DDB1, cullin E3 ligase, and proteasome.

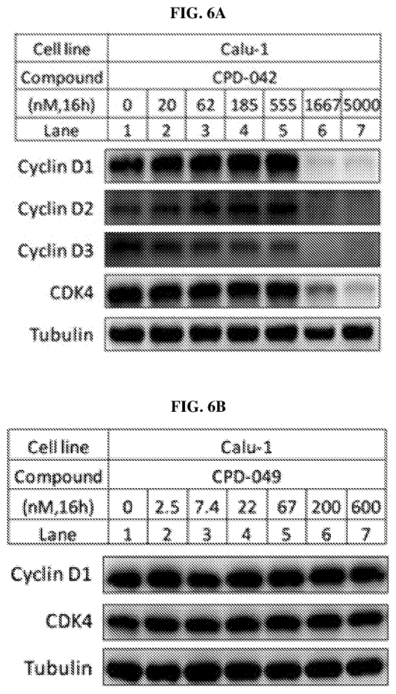

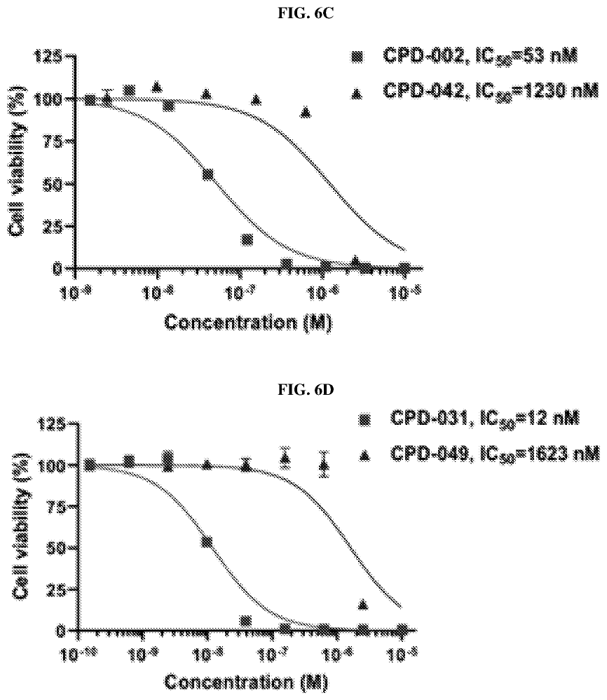

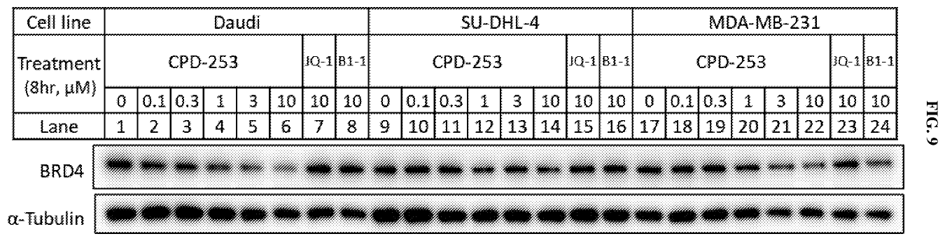

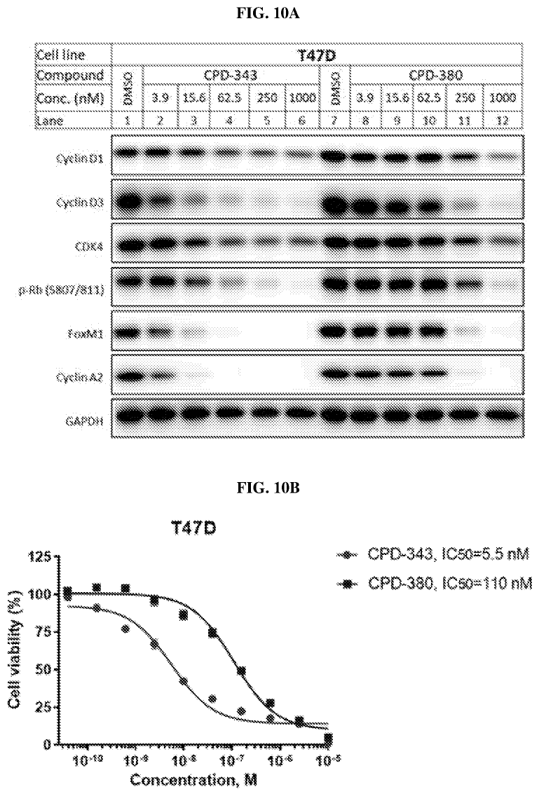

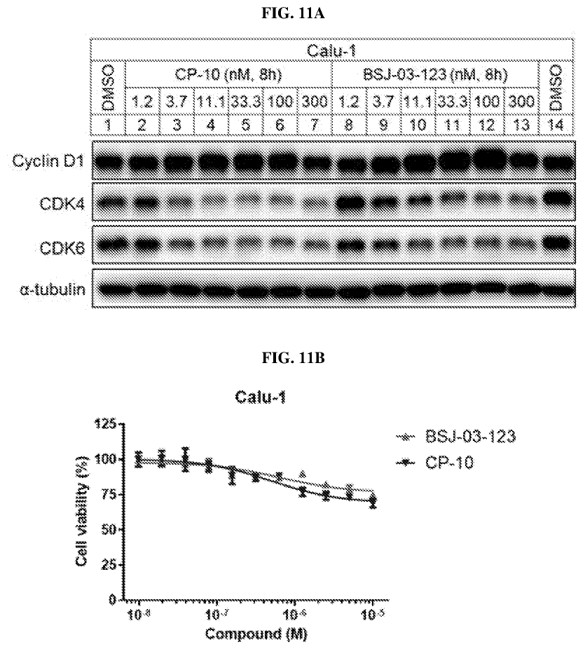

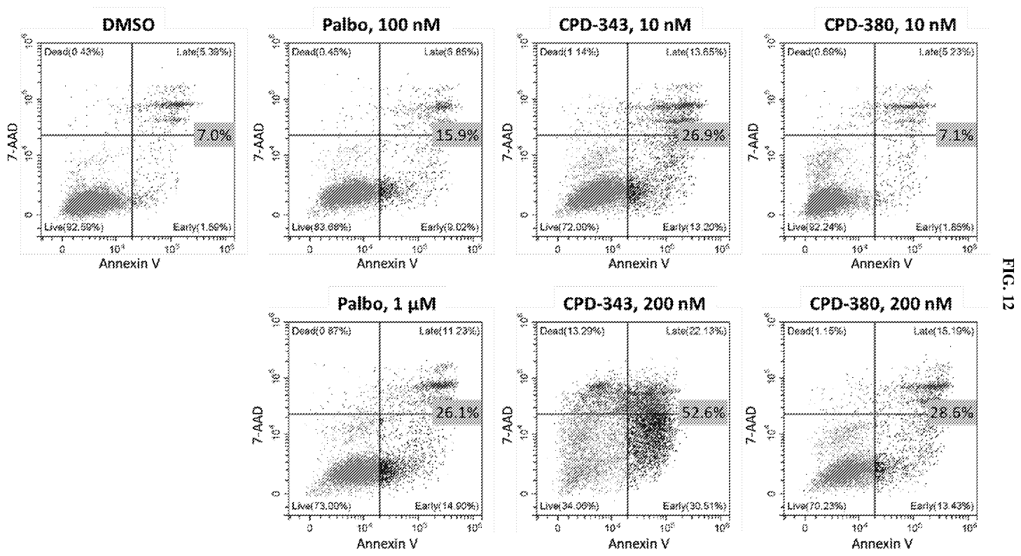

To verify that heterobifunctional compound-mediated degradation is dependent on the binding to CDK4-cyclin D complex, we designed three negative control compounds, CPD-042, CPD-049 and CPD-380, which are derived from CPD-002 CPD-031 and CPD-343, respectively. These three control compounds bear the same DDB1 ligand and linker as their corresponding active heterobifunctional compounds but with modified warheads to impair the binding of the control compounds to CDK4. As illustrated in FIG. 6A-6D and FIG. 10, compared with the corresponding active heterobifunctional compounds, the negative control compounds showed much weaker degradation potencies (>10-fold decrease for CPD-042; >100-fold decrease for CPD-049; >15-fold decrease for CPD-380) and cellular anti-proliferation activities (>20-fold decrease for CPD-042; >100-fold decrease for CPD-049; >20-fold decrease for CPD-380). These results confirm that heterobifunctional compound-mediated cyclin D and CDK4/6 degradation is dependent on their direct binding to CDK4. However, the binding to CDK4 is not sufficient for cyclin D degradation. Two cereblon (CRBN)-recruiting reference heterobifunctional compounds, CP-10 (Su, J Med Chem, 2019; CAS No.: 2366268-80-4) and BSJ-03-123 (Brand, Cell Chem Biol, 2019; CAS No.: 2361493-16-3) were analyzed in Calu-1 cells. In line with reported data, these two reference heterobifunctional compounds significantly reduced CDK4 and CDK6 protein levels but did not affect cyclin D1 protein levels (FIG. 11A) or suppress Calu-1 cell growth (FIG. 11B).

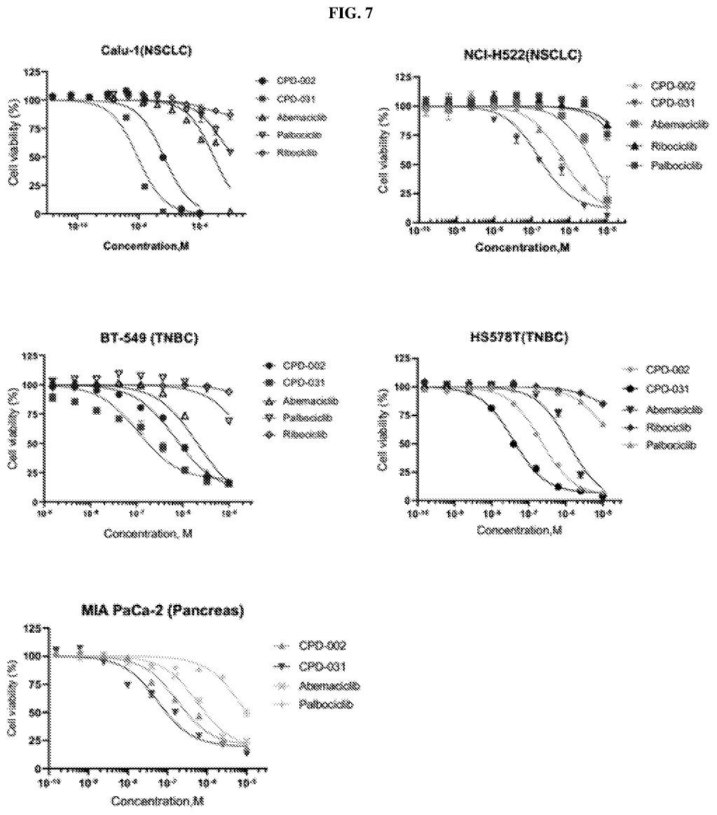

To demonstrate the advantages of our cyclin D degraders over FDA approved CDK4/6 drugs at the inhibition of cancer cell growth, Calu-1, NCI-H522, BT-549, Hs578T, MIA PaCa-2 or other cells were seeded in 96-well plates and treated with CDK4/6 inhibitors palbociclib, ribociclib, or abemaciclib, or heterobifunctional compounds CPD-002, CPD-031, CPD-043, or CPD-044 following a 9-point serial dilution after 3 d treatment. As illustrated in FIG. 7 and Table 7, CPD-002, CPD-031 are significantly more potent than palbociclib, ribociclib, and abemaciclib at the inhibition of multiple cancer cell lines.

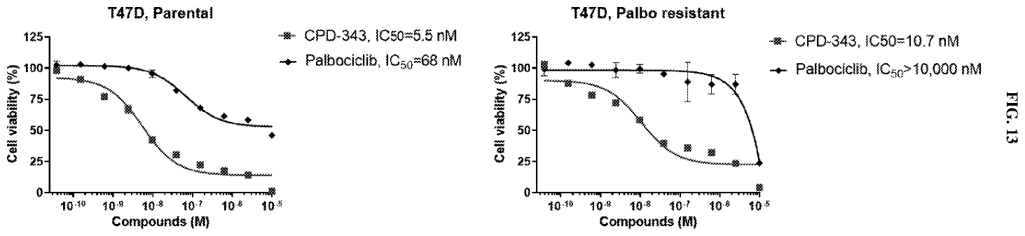

Moreover, flow cytometric analysis of Annexin V/7-AAD stained T47D cells demonstrated that our cyclin D degraders inhibited tumor cell growth by a different MoA (Mechanism of action) from CDK4/6 inhibitors. ER⁺ breast cancer T47D cells were treated with DMSO, palbociclib, heterobifunctional compound CPD-343, or negative control compound CPD-380 for 6 days at doses approximating IC₅₀ and IC₉₀ concentrations determined in FIGS. 10A and 13. Cells were harvested by trypsinization, staining was carried out using the Annexin V Apoptosis Detection Kit. The percentages of early apoptotic (Annexin V⁺7-AAD⁻, lower right quadrant), late apoptotic (Annexin V⁺7-AAD⁺, upper right quadrant) and necrotic cells (Annexin V-7-AAD⁺, upper left quadrant) are indicated on dot plots. As illustrated in FIG. 12, heterobifunctional compound CPD-343 was found to cause significant cell apoptosis at both doses approximating IC₅₀ and IC₉₀ concentrations in T47D cells (Annexin V⁺ population, 26.9% at 10 nM; 52.6% at 200 nM), while CDK4/6 inhibitor palbociclib (“palbo”) or negative control compound CPD-380 showed much less effect on cell apoptosis even at the concentration up to 1 μM (palbo: 15.9% at 100 nM; 26.1% at 1 μM; CPD-380: 7.1% at 10 nM; 28.6% at 200 nM), compared to DMSO treated cells. Furthermore, we developed one ER⁺ breast cancer T47D model with acquired resistance after long period of treatment with 1 μM palbociclib (over IC₉₀). Cells were deemed resistant when growing in the presence of palbo at the same rate as parental cells. Palbo resistance was determined by CellTiter-Lumi cell viability assay. Heterobifunctional compound CPD-343 was found to remain effective in T47D palbo-resistant model compared to parental cells (FIG. 13).

Taken together, these results indicated that degradation of cyclin D proteins could therapeutically target multiple cancer types beyond breast cancer and demonstrate more potent capability than CDK4/6 inhibitors.

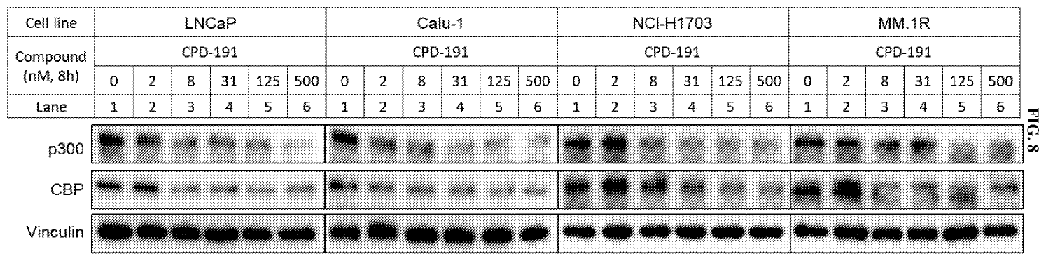

Additional exemplary heterobifunctional compounds were designed to modulate the protein levels of either P300/CBP, or BRD4, and characterized in multiple cell lines. As illustrated in FIG. 8, heterobifunctional compound CPD-191 significantly reduced P300 and CBP protein levels in a concentration-dependent manner in LNCaP, Calu-1, NCI-H1703, or MM.1R cell lines (DC₅₀<10 nM). Furthermore, specific heterobifunctional compound CPD-253 was found to dramatically reduce BRD4 protein levels in Daudi, SU-DHL-4, or MDA-MB-231 cell lines (FIG. 9). Taken together, DDB1 ligands conjugating with different target ligands may modulate the cellular target protein levels of cyclin D, CDK4/6, P300/CBP and BRD4. The data indicate a wide degree of usefulness for DDB1 ligands in targeted protein degradation technology.

### EXAMPLES

The following examples are set forth to illustrate more clearly the principle and practice of instances disclosed herein to those skilled in the art and are not to be construed as limiting the scope of any claimed instances. Unless otherwise stated, all parts and percentages are on a weight basis.

### General Chemistry Methods

All chemicals and reagents were purchased from commercial suppliers and used without further purification. LCMS spectra for all compounds were acquired using a Waters LC-MS AcQuity H UPLC class system. The Waters LC-MS AcQuity H UPLC class system comprising a pump (Quaternary Solvent Manager) with degasser, an autosampler (FTN), a column oven (40° C., unless otherwise indicated), a photo-diode array PDA detector. Chromatography was performed on an AcQuity UPLC BEH C18 (1.7 μm, 2.1×50 mm) with water containing 0.1% formic acid as solvent A and acetonitrile containing 0.1% formic acid as solvent B at a flow rate of 0.6 mL/min. Flow from the column was split to a MS spectrometer. The MS detector was configured with an electrospray ionization source. Nitrogen was used as the nebulizer gas. Data acquisition was performed with a MassLynx data system. Nuclear Magnetic Resonance spectra were recorded on a Bruker Avance 111400 spectrometer. Chemical shifts are expressed in parts per million (ppm) and reported as 6 value (chemical shift 6). Coupling constants are reported in units of hertz (J value, Hz; Integration and splitting patterns: where s=singlet, d=double, t=triplet, q=quartet, brs=broad singlet, m=multiple). The purification of intermediates or final products were performed on Agilent Prep 1260 series with UV detector set to 254 nm or 220 nm. Samples were injected onto a Phenomenex Luna C18 column (5 μm, 30×75 mm) at room temperature. The flow rate was 40 mL/min. A linear gradient was used with either 10% or 50% MeOH in H₂O containing 0.1% TFA as solvent A and 100% of MeOH as solvent B. Alternatively, the products were purified on CombiFlash® NextGen 300 system with UV detector set to 254 nm, 220 nm or 280 nm. The flow rate was 40 mL/min. A linear gradient was used with H₂O containing 0.05% TFA as solvent A and 100% of MeOH containing 0.05% TFA as solvent B. All compounds showed >95% purity using the LCMS methods described above.

The following are non-limiting examples of a synthesis of ligands.

### Example 001. 4-((2,2-Dimethyl-4-oxo-3,8,11,14,17,20-hexaoxa-5-azadocosan-22-yl)amino)-2-methylbenzoic acid (BL1-1)

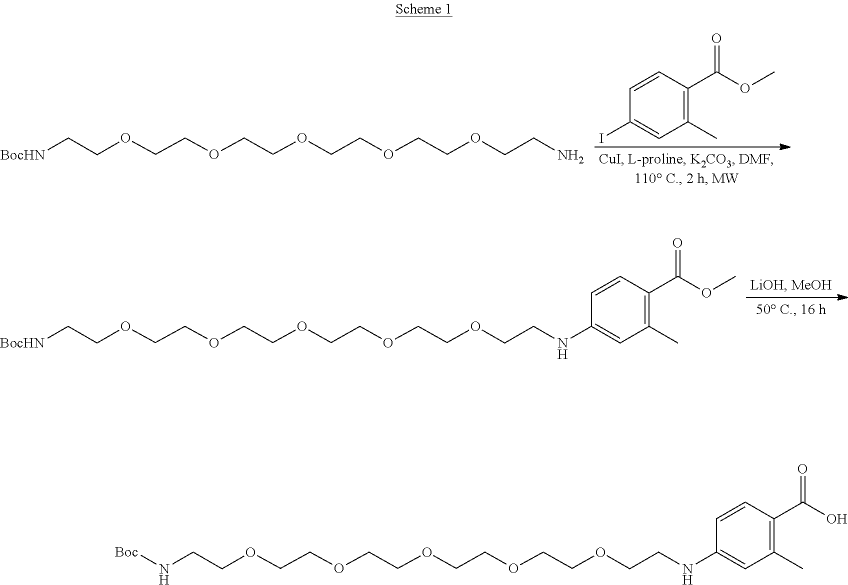

### Step 1. Synthesis of methyl 4-((2,2-dimethyl-4-oxo-3,8,11,14,17,20-hexaoxa-5-azadocosan-22-yl)amino)-2-methylbenzoate

A solution of tert-butyl (17-amino-3,6,9,12,15-pentaoxaheptadecyl)carbamate (2.00 g, 5.26 mmol), L-proline (605 mg, 5.26 mmol), K₂CO₃ (1.45 g, 10.5 mmol), CuI (1.00 g, 5.26 mmol) and methyl 4-iodo-2-methylbenzoate (1.74 g, 6.31 mmol) in DMF (20 mL) was stirred at 110° C. for 2 h under microwave irradiation in argon atmosphere. After cooling down to rt, the mixture was diluted with water (100 mL) and extracted with EtOAc (2×100 mL). The combined organic phase was washed with brine (2×100 mL), dried over Na₂SO₄, filtered and concentrated under reduced pressure. The residue was purified by silica gel column chromatography (petroleum ether/EtOAc=5:1) to provide the desired product (1.20 g, 43% yield) as a colorless oil. MS (ESI) m/z=529.2 [M+H]⁺.

### Step 2. Synthesis of 4-((2,2-dimethyl-4-oxo-3,8,11,14,17,20-hexaoxa-5-azadocosan-22-yl)amino)-2-methylbenzoic acid

A solution of methyl 4-((2,2-dimethyl-4-oxo-3,8,11,14,17,20-hexaoxa-5-azadocosan-22-yl)amino)-2-methylbenzoate (1.20 g, 2.27 mmol) and LiOH·H₂O (477 mg, 11.4 mmol) in MeOH (10 ml) and H₂O (1 ml) was stirred at 50° C. for 16 h. After cooling down to rt, the mixture was diluted with water (50 mL), and adjusted pH to 4 with 1N HCl. The mixture was extracted with EtOAc (2×50 mL). The combined organic phase was washed with brine (2×50 mL), dried over Na₂SO₄, filtered and concentrated under vacuum to provide the crude title compound (1.05 g, 90% yield) as a brown oil. ¹HNMR (400 MHz, DMSO-d₆) δ 11.77 (s, 1H), 7.66 (d, J=8.4 Hz, 1H), 6.74 (t, J=5.2 Hz, 1H), 6.43-6.41 (m, 2H), 6.25 (t, J=5.6 Hz, 1H), 3.56-3.48 (m, 18H), 3.36 (t, J=6.0 Hz, 2H), 3.23 (q, J=5.6 Hz, 2H), 3.05 (q, J=5.6 Hz, 2H), 2.43 (s, 3H), 1.36 (s, 9H). MS (ESI) m/z=515.3 [M+H]⁺.

### Example 002. N⁴-(5-Aminopentyl)-2-methyl-N¹-(5-methylthiazol-2-yl)terephthalamide (BL1-2)

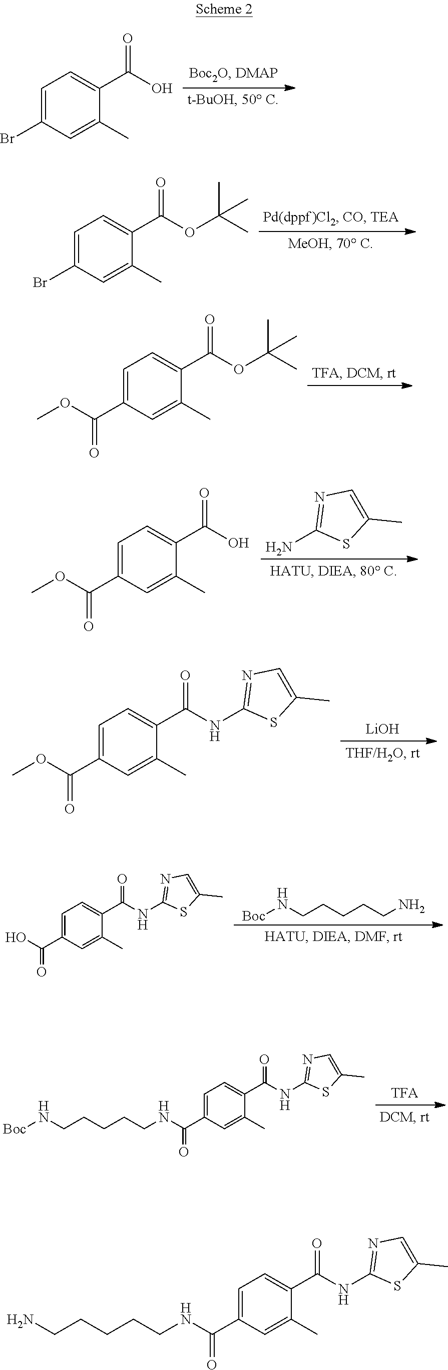

### Step 1. Synthesis of tert-butyl 4-bromo-2-methylbenzoate

A solution of 4-bromo-2-methylbenzoic acid (10 g, 46.5 mmol), DMAP (567 mg, 4.65 mmol) and Boc₂O (15.2 g, 69.8 mmol) in t-BuOH (100 mL) was stirred at 50° C. overnight. After cooling down to rt, the mixture was concentrated and purified by silica gel column chromatography (petroleum ether/EtOAc=10:1) to provide the title compound (8.0 g, 64% yield) as a colorless oil.

### Step 2. Synthesis of 1-(tert-butyl) 4-methyl 2-methylterephthalate

A solution of tert-butyl 4-bromo-2-methylbenzoate (8.00 g, 29.5 mmol), Pd(dppf)Cl₂ (2.16 g, 2.95 mmol) and TEA (5.96 g, 59.0 mmol) in MeOH (80 mL) was heated at 70° C. under carbon monoxide atmosphere (15 psi) overnight. After cooling down to rt, the mixture was concentrated under reduced pressure. The residue was diluted with ethyl acetate (100 mL) and washed with brine (2×30 mL). The organic phase was dried over anhydrous Na₂SO₄, filtered and concentrated. The residue was purified by silica gel column chromatography (petroleum ether/EtOAc=10:1) to provide the desired product (6.0 g, 81% yield) as a colorless oil.

### Step 3. Synthesis of 4-(methoxycarbonyl)-2-methylbenzoic acid

A solution of 1-(tert-butyl) 4-methyl 2-methylterephthalate (6.00 g, 24.0 mmol) in DCM (20 mL) and TFA (20 mL) was stirred at rt overnight. The reaction mixture was concentrated under vacuum and lyophilized to provide the title compound (4.20 g, 90% yield) as a white solid. MS (ESI) m/z=193.0 [M−H]⁻.

### Step 4. Synthesis of methyl 3-methyl-4-((5-methylthiazol-2-yl)carbamoyl)benzoate

A solution of 4-(methoxycarbonyl)-2-methylbenzoic acid (4.20 g, 21.6 mmol), 5-methylthiazol-2-amine (3.69 g, 32.4 mmol), HATU (12.3 g, 32.4 mmol) and DIEA (8.36 g, 64.8 mmol) in DMF (50 mL) was stirred at 80° C. for 2 h. After cooling down to rt, the mixture was diluted with water (200 mL) and acidified with 1N HCl to pH=5. The mixture was filtered and the filter cake was washed with MeOH (100 mL). The solid was dried under high vacuum to provide the title compound (3.00 g, 48% yield) as a pale-yellow solid. MS (ESI) m/z=291.1 [M+H]⁺.

### Step 5. Synthesis of 3-methyl-4-((5-methylthiazol-2-yl)carbamoyl)benzoic acid

A solution of methyl 3-methyl-4-((5-methylthiazol-2-yl)carbamoyl)benzoate (3.00 g, 10.3 mmol) and LiOH·H₂O (2.16 g, 51.5 mmol) in THF (50 mL) and H₂O (20 mL) was stirred at rt overnight. The mixture was concentrated under vacuum to remove THF. The residue was diluted with water (100 mL) and acidified with 1N HCl to pH=2. The mixture was filtered and the filter cake was lyophilized to provide the title compound (2.50 g, 88% yield) as a white solid. ¹HNMR (400 MHz, DMSO-d₆) δ 12.8 (brs, 2H), 7.87 (s, 1H), 7.83 (dd, J=8.0, 0.8 Hz, 1H), 7.61 (d, J=8.0 Hz, 1H), 7.20 (d, J=1.2 Hz, 1H), 2.42 (s, 3H), 2.38 (s, 3H). MS (ESI) m/z=277.0 [M+H]⁺.

### Step 6. Synthesis of tert-butyl (5-(3-methyl-4-((5-methylthiazol-2-yl)carbamoyl)benzamido)pentyl)carbamate

A solution of 3-methyl-4-((5-methylthiazol-2-yl)carbamoyl)benzoic acid (200 mg, 0.725 mmol), tert-butyl (5-aminopentyl)carbamate (184 mg, 0.906 mmol), HATU (413 mg, 1.09 mmol) and DIEA (280 mg, 2.18 mmol) in DMF (8 mL) was stirred at rt overnight. The mixture was diluted with water (50 mL) and extracted with EtOAc (3×50 mL). The combined organic phase was washed with brine (2×100 mL), dried over Na₂SO₄, filtered and concentrated under vacuum. The residue was purified by prep-HPLC to provide the title compound (150 mg, 45% yield) as a yellow oil. MS (ESI) m/z=461.2 [M+H]⁺.

### Step 7. Synthesis of N⁴-(5-aminopentyl)-2-methyl-N¹-(5-methylthiazol-2-yl)terephthalamide

A solution of tert-butyl (5-(3-methyl-4-((5-methylthiazol-2-yl)carbamoyl)benzamido)pentyl)carbamate (150 mg, 0.326 mmol) in DCM (5 mL) and TFA (2 mL) was stirred at rt for 2 h. The mixture was concentrated and lyophilized to provide the title compound (130 mg, TFA salt, 84% yield) as a yellow solid. ¹HNMR (400 MHz, DMSO-d₆) δ 12.39 (brs, 1H), 8.54 (t, J=5.2 Hz, 1H), 7.76-7.67 (m, 4H), 7.59 (d, J=8.0 Hz, 1H), 7.20 (d, J=1.2 Hz, 1H), 3.29-3.25 (m, 2H), 2.81-2.77 (m, 2H), 2.42 (s, 3H), 2.38 (s, 3H), 1.59-1.53 (m, 4H), 1.37-1.33 (m, 2H). MS (ESI) m/z=361.2 [M+H]⁺.

### Example 003. N⁴-(7-Aminoheptyl)-2-methyl-N¹-(5-methylthiazol-2-yl)terephthalamide (BL1-3)

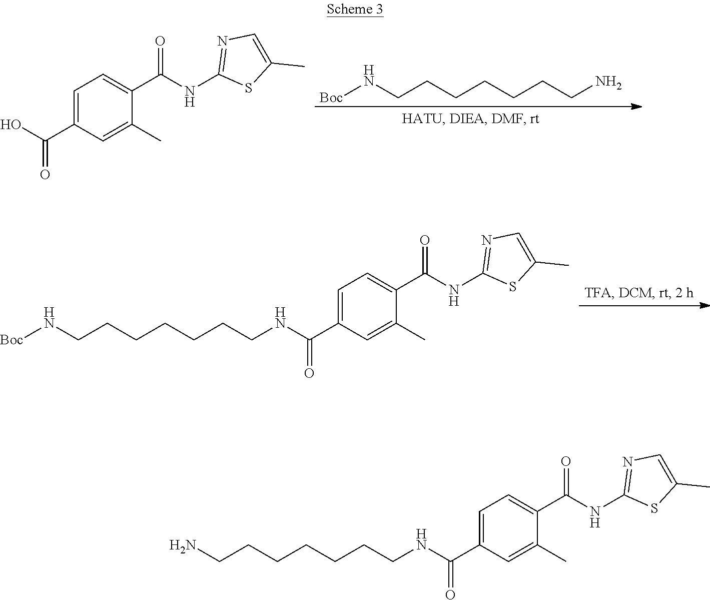 [SMILES: Cc1cnc(NC(=O)c2ccc(C(=O)O)cc2C)s1 ; CC(C)(C)OC(=O)NCCCCCCCN ; Cc1cnc(NC(=O)c2ccc(C(=O)NCCCCCCCNC(=O)OC(C)(C)C)cc2C)s1 ; Cc1cnc(NC(=O)c2ccc(C(=O)NCCCCCCCN)cc2C)s1]

BL1-3 was synthesized following the standard procedures for preparing BL1-2 (120 mg, 36% yield over two steps). ¹HNMR (400 MHz, DMSO-d₆) δ 12.39 (brs, 1H), 8.54 (t, J=5.2 Hz, 1H), 7.76 (s, 1H), 7.71-7.70 (m, 1H), 7.65-7.56 (m, 3H), 7.20 (d, J=1.2 Hz, 1H), 3.27-3.24 (m, 2H), 2.79-2.74 (m, 2H), 2.42 (s, 3H), 2.38 (s, 3H), 1.54-1.51 (m, 4H), 1.31-1.28 (m, 6H). MS (ESI) m/z=389.1 [M+H]⁺.

### Example 004. N⁴-(9-Aminononyl)-2-methyl-N¹-(5-methylthiazol-2-yl)terephthalamide (BL1-4)

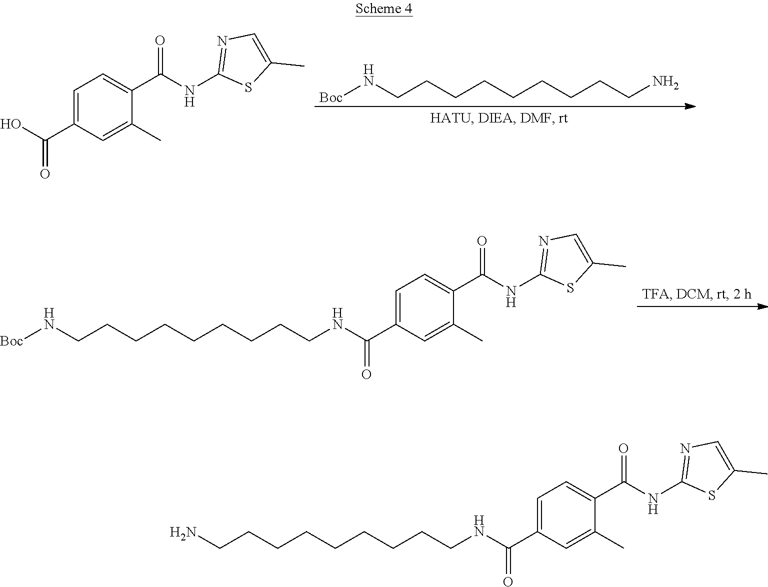 [SMILES: Cc1cnc(NC(=O)c2ccc(C(=O)O)cc2C)s1 ; CC(C)(C)OC(=O)NCCCCCCCCCN ; Cc1cnc(NC(=O)c2ccc(C(=O)NCCCCCCCCCNC(=O)OC(C)(C)C)cc2C)s1 ; Cc1cnc(NC(=O)c2ccc(C(=O)NCCCCCCCCCN)cc2C)s1]

BL1-4 was synthesized following the standard procedures for preparing BL1-2 (150 mg, 41% yield over two steps). ¹HNMR (400 MHz, DMSO-d₆) δ 12.42 (brs, 1H), 8.53 (t, J=5.6 Hz, 1H), 7.75 (s, 1H), 7.72-7.70 (m, 1H), 7.64-7.58 (m, 3H), 7.20 (d, J=1.2 Hz, 1H), 3.28-3.23 (m, 2H), 2.79-2.74 (m, 2H), 2.41 (s, 3H), 2.38 (s, 3H), 1.52-1.49 (m, 4H), 1.28-1.22 (m, 10H). MS (ESI) m/z=417.2 [M+H]⁺.

### Example 005. N⁴-(2-(2-(2-Aminoethoxy)ethoxy)ethyl)-2-methyl-N¹-(5-methylthiazol-2-yl)terephthalamide (BL1-5)

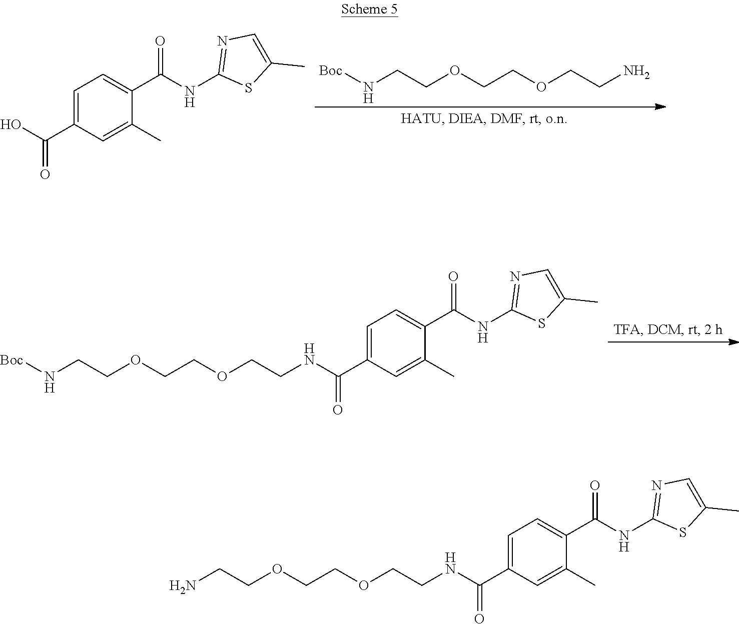

BL1-5 was synthesized following the standard procedures for preparing BL1-2 (65 mg, 23% yield over two steps). ¹HNMR (400 MHz, DMSO-d₆) δ 12.44 (brs, 1H), 8.63 (t, J=5.6 Hz, 1H), 7.79-7.72 (m, 4H), 7.60 (d, J=8.0 Hz, 1H), 7.21 (d, J=1.2 Hz, 1H), 3.60-3.56 (m, 8H), 3.46-3.42 (m, 2H), 2.98-2.95 (m, 2H), 2.42 (s, 3H), 2.38 (s, 3H). MS (ESI) m/z=407.2 [M+H]⁺.

### Example 006. N⁴-(2-(2-(2-(2-Aminoethoxy)ethoxy)ethoxy)ethyl)-2-methyl-N¹-(5-methylthiazol-2-yl)terephthalamide (BL1-6)

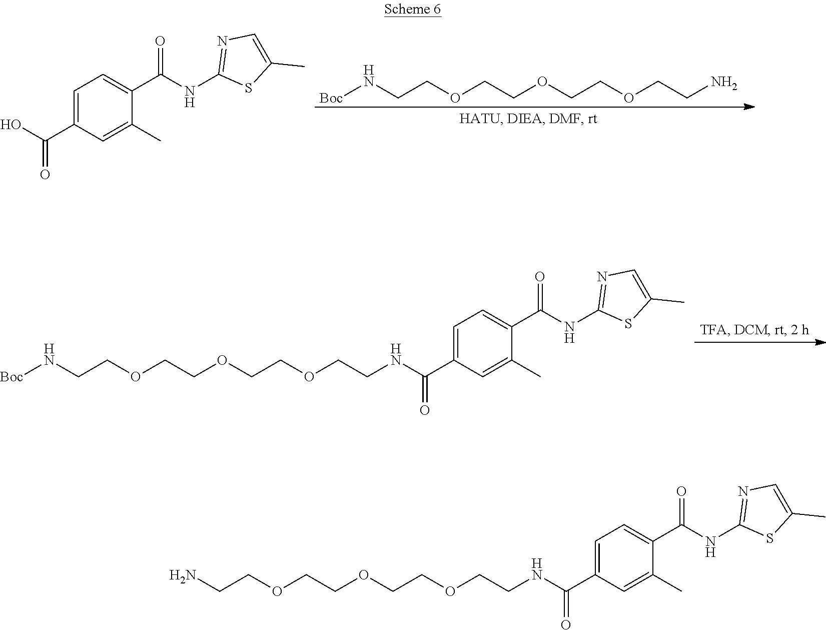 [SMILES: Cc1cnc(NC(=O)c2ccc(C(=O)O)cc2C)s1 ; CC(C)(C)OC(=O)NCCOCCOCCOCCN ; Cc1cnc(NC(=O)c2ccc(C(=O)NCCOCCOCCOCCNC(=O)OC(C)(C)C)cc2C)s1 ; Cc1cnc(NC(=O)c2ccc(C(=O)NCCOCCOCCOCCN)cc2C)s1]

BL1-6 was synthesized following the standard procedures for preparing BL1-2 (140 mg, 34% yield over two steps). ¹HNMR (400 MHz, DMSO-d₆) δ 12.42 (brs, 1H), 8.62 (t, J=5.2 Hz, 1H), 7.70-7.68 (m, 4H), 7.60 (d, J=8.0 Hz, 1H), 7.20 (d, J=1.2 Hz, 1H), 3.59-3.54 (m, 12H), 3.46-3.43 (m, 2H), 2.99-2.95 (m, 2H), 2.42 (s, 3H), 2.38 (s, 3H). MS (ESI) m/z=451.3 [M+H]⁺.

### Example 007. N⁴-(14-Amino-3,6,9,12-tetraoxatetradecyl)-2-methyl-N¹-(5-methylthiazol-2-yl)terephthalamide (BL1-7)

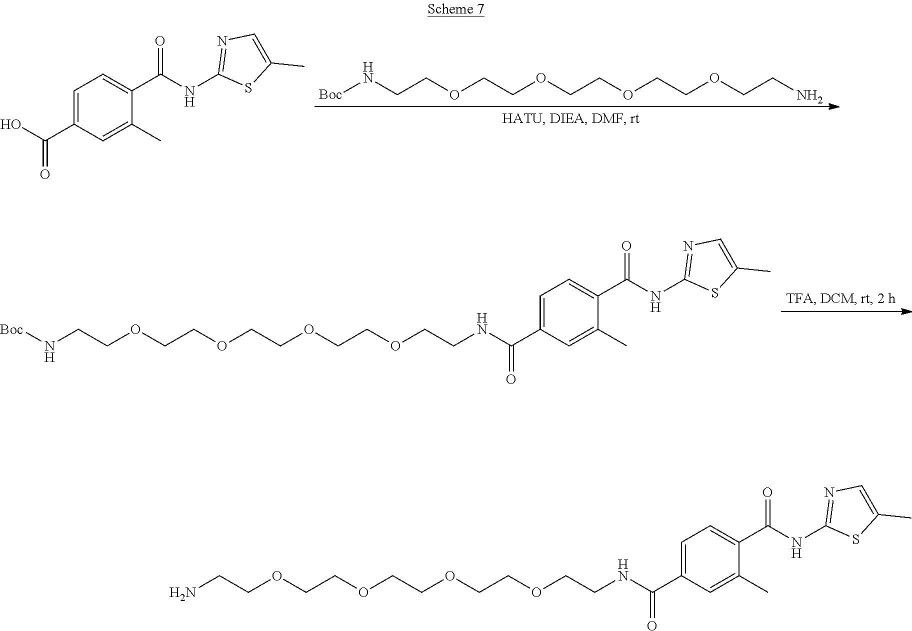

BL1-7 was synthesized following the standard procedures for preparing BL1-2 (91 mg, 21% yield over two steps). ¹HNMR (400 MHz, DMSO-d₆) δ 12.39 (brs, 1H), 8.61 (t, J=5.6 Hz, 1H), 7.77-7.61 (m, 4H), 7.60 (d, J=8.0 Hz, 1H), 7.20 (d, J=1.2 Hz, 1H), 3.60-3.52 (m, 16H), 3.45-3.41 (m, 2H), 2.99-2.96 (m, 2H), 2.42 (s, 3H), 2.38 (s, 3H). MS (ESI) m/z=495.2 [M+H]⁺.

### Example 008. N⁴-(17-Amino-3,6,9,12,15-pentaoxaheptadecyl)-2-methyl-N¹-(5-methylthiazol-2-yl)terephthalamide (BL1-8)

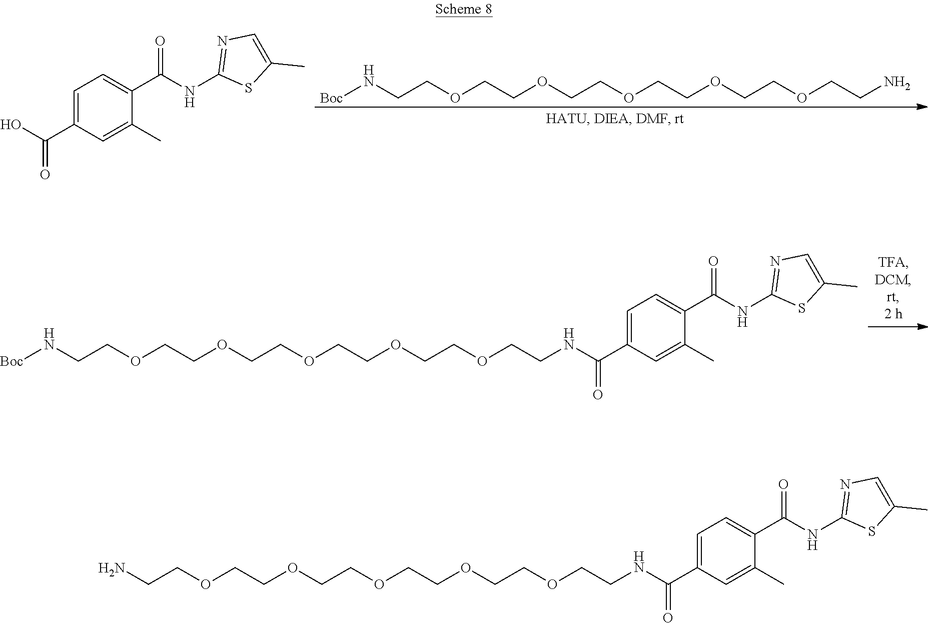 [SMILES: Cc1cnc(NC(=O)c2ccc(C(=O)O)cc2C)s1 ; CC(C)(C)OC(=O)NCCOCCOCCOCCOCCOCCN ; Cc1cnc(NC(=O)c2ccc(C(=O)NCCOCCOCCOCCOCCOCCNC(=O)OC(C)(C)C)cc2C)s1 ; Cc1cnc(NC(=O)c2ccc(C(=O)NCCOCCOCCOCCOCCOCCN)cc2C)s1]

BL1-8 was synthesized following the standard procedures for preparing BL1-2 (240 mg, 51% yield over two steps). ¹HNMR (400 MHz, DMSO-d₆) δ 12.4 (brs, 1H), 8.62 (t, J=5.6 Hz, 1H), 7.78-7.72 (m, 4H), 7.59 (d, J=8.0 Hz, 1H), 7.21 (s, 1H), 3.60-3.50 (m, 20H), 3.46-3.41 (m, 2H), 2.99-2.95 (m, 2H), 2.42 (s, 3H), 2.38 (s, 3H). MS (ESI) m/z=539.3 [M+H]⁺.

### Example 009. 4-((2-((5-Aminopentyl)amino)-2-oxoethyl)amino)-2-methyl-N-(5-methylthiazol-2-yl)benzamide (BL1-9)

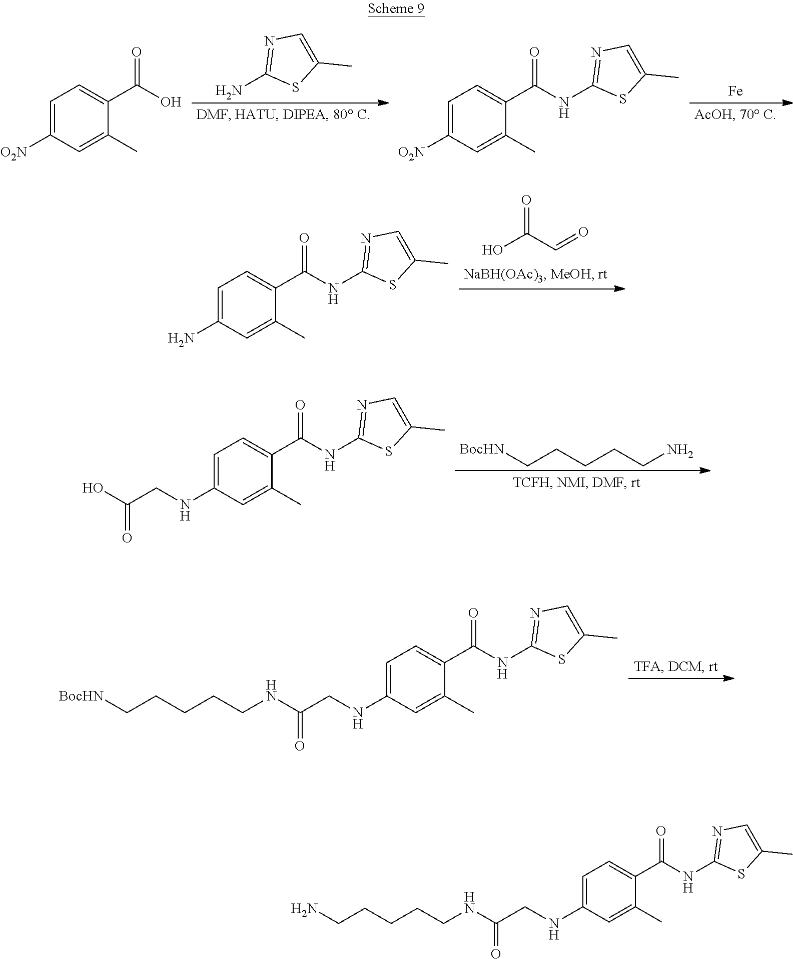

### Step 1. Synthesis of 2-methyl-N-(5-methylthiazol-2-yl)-4-nitrobenzamide

To a solution of 2-methyl-4-nitrobenzoic acid (5.00 g, 27.6 mmol) in DMF (100 mL) were added 5-methylthiazol-2-amine (3.20 g, 28.0 mmol), HATU (11.4 g, 30.0 mmol) and DIPEA (7.74 g, 60.0 mmol). The reaction mixture was stirred at 80° C. for 2 h. After cooling down to rt, the solution was poured into ice-water (500 mL). The solid was collected by filtration, washed with H₂O, and dried over vacuum to afford the title compound (7.0 g, 92% yield) as a yellow solid. MS (ESI) m/z=278.0 [M+H]⁺.

### Step 2. Synthesis of 4-amino-2-methyl-N-(5-methylthiazol-2-yl)benzamide

To a solution of 2-methyl-N-(5-methylthiazol-2-yl)-4-nitrobenzamide (7.00 g, 25.2 mmol) in AcOH (50 mL) was added iron powder (11.2 g, 200 mmol). After stirring at 70° C. for 2 h, the reaction mixture was diluted with H₂O (20 mL), filtered and concentrated under reduced pressure. The residue was adjusted with aq.NaHCO₃ to pH=6. The solid was collected by filtration and washed with H₂O, dried over vacuum to afford the title compound (6.00 g, 96% yield) as a yellow solid. ¹HNMR (400 MHz, DMSO-d₆) δ 11.17 (s, 1H), 7.38-7.36 (m, 1H), 7.14 (s, 1H), 6.40 (s, 2H), 5.62 (s, 2H), 2.36 (s, 6H). MS (ESI) m/z=248.0 [M+H]⁺.

### Step 3. Synthesis of (3-methyl-4-((5-methylthiazol-2-yl)carbamoyl)phenyl)glycine

To a solution of 4-amino-2-methyl-N-(5-methylthiazol-2-yl)benzamide (2.5 g, 7.20 mmol) in MeOH (50 mL) were added NaBH(OAc)₃ (3.04 g, 14.4 mmol) and 2-oxoacetic acid (50%, 2 ml). After stirring at rt overnight, the solid was collected by filtration, washed with MeOH, and dried over vacuum to afford the title compound (1.7 g, 77% yield) as a yellow solid. ¹HNMR (400 MHz, DMSO-d₆) δ 12.58 (brs, 1H), 11.86 (brs, 1H), 7.45-7.44 (m, 1H), 7.14 (s, 1H), 6.45-6.44 (m, 3H), 3.87 (s, 2H), 2.36 (s, 6H). MS (ESI) m/z=306.0 [M+H]⁺.

### Step 4. Synthesis of tert-butyl (5-(2-((3-methyl-4-((5-methylthiazol-2-yl)carbamoyl)phenyl)amino)acetamido)pentyl)carbamate

To a solution of (3-methyl-4-((5-methylthiazol-2-yl)carbamoyl)phenyl)glycine (200 mg, 0.656 mmol) in DMF (2 mL) were added N,N,N′,N′-tetramethylchloroformamidinium hexafluorophosphate (TCFH) (277 mg, 0.984 mmol), N-methylimidazole (81 mg, 0.984 mmol) and tert-butyl (5-aminopentyl)carbamate (74 mg, 0.722 mmol). After the mixture was stirred at rt for 3 h, it was diluted with water (20 mL) and extracted with EtOAc (3×20 mL). The combined organic layers were washed with water and brine, dried over Na₂SO₄, filtered and concentrated under reduced pressure to provide the title compound (310 mg, crude) as a brown oil.

### Step 5. Synthesis of 4-((2-((5-aminopentyl)amino)-2-oxoethyl)amino)-2-methyl-N-(5-methylthiazol-2-yl)benzamide

To a solution of tert-butyl (5-(2-((3-methyl-4-((5-methylthiazol-2-yl)carbamoyl)phenyl)amino)acetamido)pentyl)carbamate (310 mg, crude) in DCM (2 mL) was added TFA (1 mL). After the reaction mixture was stirred at rt for 5 h, it was concentrated and purified by prep-HPLC (0.1% TFA) to provide the title compound (74.8 mg, TFA salt, 29% yield over two step) as a white solid. ¹HNMR (400 MHz, DMSO-d₆) δ 11.87 (brs, 1H), 7.96 (t, J=11.6 Hz, 1H), 7.75 (brs, 3H), 7.45 (d, J=8.4 Hz 1H), 7.14 (d, J=1.2 Hz, 1H), 6.41-6.38 (m, 2H), 3.67 (s, 2H), 3.10-3.05 (m, 2H), 2.76-2.71 (m, 2H), 2.36 (s, 3H), 2.35 (s, 3H), 1.53-1.49 (m, 2H), 1.42-1.37 (m, 2H), 1.30-1.24 (m, 2H). MS (ESI) m/z=390.2 [M+H]⁺.

### Example 010. 4-((2-((7-Aminoheptyl)amino)-2-oxoethyl)amino)-2-methyl-N-(5-methylthiazol-2-yl)benzamide (BL1-10)

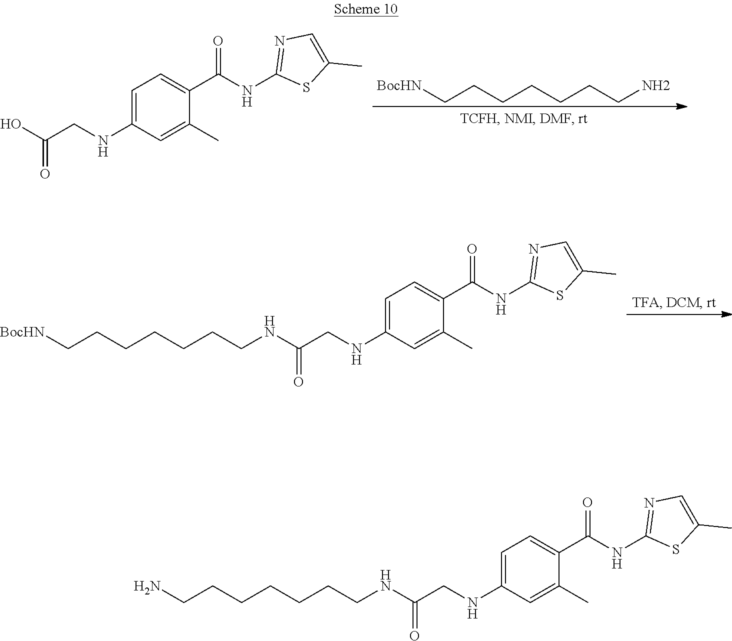 [SMILES: Cc1cnc(NC(=O)c2ccc(NCC(=O)O)cc2C)s1 ; CC(C)(C)OC(=O)NCCCCCCCN ; Cc1cnc(NC(=O)c2ccc(NCC(=O)NCCCCCCCNC(=O)OC(C)(C)C)cc2C)s1 ; "TFA, DCM, rt" ; Cc1cnc(NC(=O)c2ccc(NCC(=O)NCCCCCCCN)cc2C)s1]

BL1-10 was synthesized following the standard procedures for preparing BL1-9 (130 mg, 37% yield over two steps). ¹HNMR (400 MHz, DMSO-d₆) δ 11.85 (brs, 1H), 7.90 (t, J=11.6 Hz, 1H), 7.61 (brs, 3H), 7.44 (d, J=8.4 Hz, 1H), 7.14 (d, J=1.2 Hz, 1H), 6.40-6.38 (m, 2H), 3.86 (s, 2H), 3.09-3.05 (m, 2H), 2.77-2.72 (m, 2H), 2.36 (s, 3H), 2.35 (s, 3H), 1.53-1.49 (m, 2H), 1.42-1.37 (m, 2H), 1.30-1.24 (m, 6H). MS (ESI) m/z=418.2 [M+H]⁺.

### Example 011. 4-((2-((9-Aminononyl)amino)-2-oxoethyl)amino)-2-methyl-N-(5-methylthiazol-2-yl)benzamide (BL1-11)

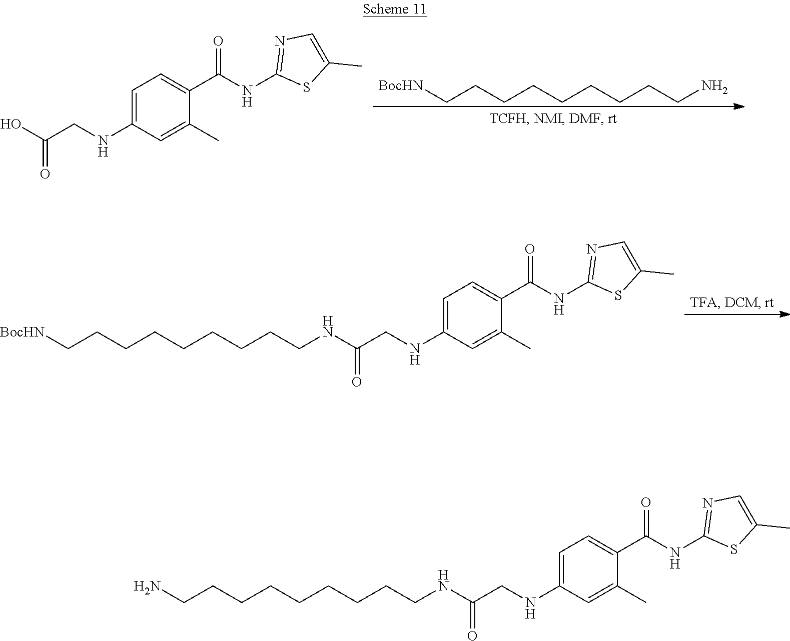 [SMILES: Cc1cnc(NC(=O)c2ccc(NCC(=O)O)cc2C)s1 ; CC(C)(C)OC(=O)NCCCCCCCCCN ; Cc1cnc(NC(=O)c2ccc(NCC(=O)NCCCCCCCCCNC(=O)OC(C)(C)C)cc2C)s1 ; "TFA, DCM, rt" ; Cc1cnc(NC(=O)c2ccc(NCC(=O)NCCCCCCCCCN)cc2C)s1]

BL1-11 was synthesized following the standard procedures for preparing BL1-9 (48 mg, 14% yield over two steps). ¹HNMR (400 MHz, DMSO-d₆) δ 11.85 (brs, 1H), 7.88 (t, J=11.6 Hz, 1H), 7.63 (brs, 3H), 7.44 (d, J=8.4 Hz, 1H), 7.14 (d, J=1.2 Hz, 1H), 6.40-6.38 (m, 2H), 3.66 (s, 2H), 3.09-3.05 (m, 2H), 2.77-2.72 (m, 2H), 2.36 (s, 3H), 2.34 (s, 3H), 1.53-1.47 (m, 2H), 1.42-1.37 (m, 2H), 1.36-1.24 (m, 10H). MS (ESI) m/z=446.2 [M+H]⁺.

### Example 012. 4-((2-((2-(2-(2-Aminoethoxy)ethoxy)ethyl)amino)-2-oxoethyl)amino)-2-methyl-N-(5-methylthiazol-2-yl)benzamide (BL1-12)

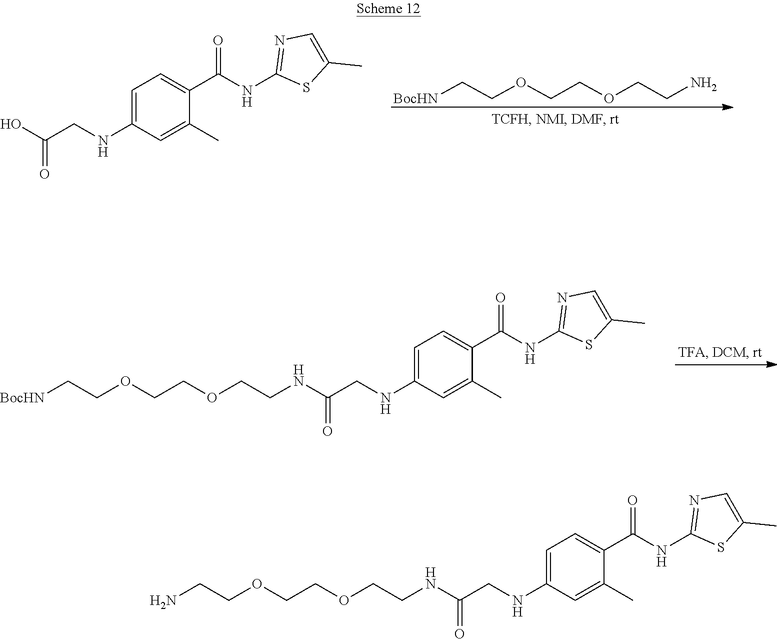 [SMILES: Cc1cnc(NC(=O)c2ccc(NCC(=O)O)cc2C)s1 ; CC(C)(C)OC(=O)NCCOCCOCCN ; Cc1cnc(NC(=O)c2ccc(NCC(=O)NCCOCCOCCNC(=O)OC(C)(C)C)cc2C)s1 ; Cc1cnc(NC(=O)c2ccc(NCC(=O)NCCOCCOCCN)cc2C)s1]

BL1-12 was synthesized following the standard procedures for preparing BL1-9 (168 mg, 59% yield over two steps). ¹HNMR (400 MHz, DMSO-d₆) δ 11.86 (brs, 1H), 7.95 (t, J=5.6 Hz, 1H), 7.77 (brs, 3H), 7.44 (d, J=8.4 Hz, 1H), 7.14 (s, 1H), 6.41-6.37 (m, 2H), 3.69 (s, 2H), 3.58-3.52 (m, 6H), 3.42-3.39 (m, 2H), 3.27-3.22 (m, 2H), 2.98-2.96 (m, 2H), 2.36 (s, 3H), 2.34 (s, 3H). MS (ESI) m/z=480.2 [M+H]⁺.

### Example 013. 4-((14-Amino-2-oxo-6,9,12-trioxa-3-azatetradecyl)amino)-2-methyl-N-(5-methylthiazol-2-yl)benzamide (BL1-13)

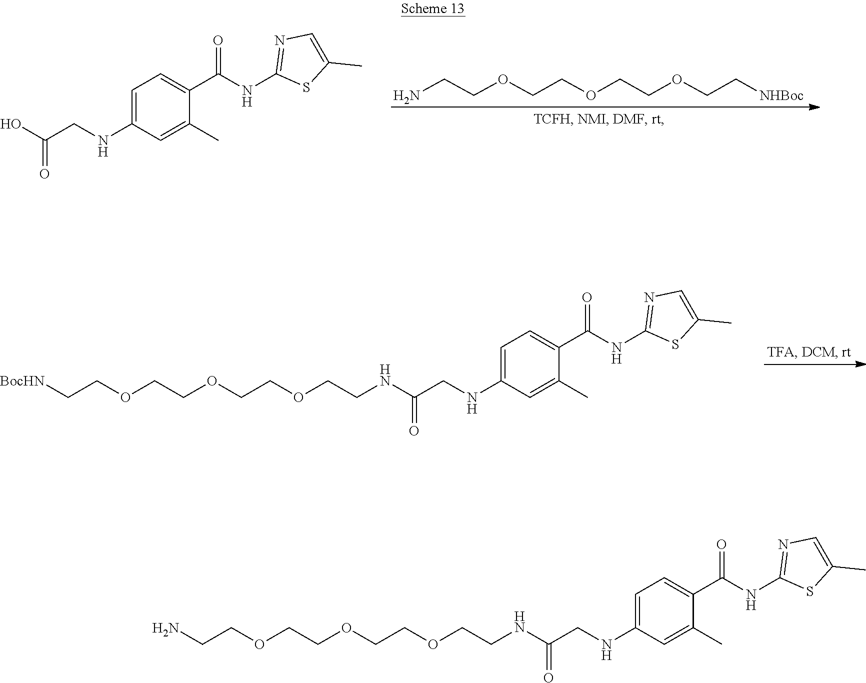 [SMILES: Cc1cnc(NC(=O)c2ccc(NCC(=O)O)cc2C)s1 ; CC(C)(C)OC(=O)NCCOCCOCCOCCN ; Cc1cnc(NC(=O)c2ccc(NCC(=O)NCCOCCOCCOCCNC(=O)OC(C)(C)C)cc2C)s1 ; Cc1cnc(NC(=O)c2ccc(NCC(=O)NCCOCCOCCOCCN)cc2C)s1]

BL1-13 was synthesized following the standard procedures for preparing BL1-9 (130 mg, 44% yield over two steps). ¹HNMR (400 MHz, DMSO-d₆) δ 11.86 (brs, 1H), 7.95 (t, J=5.6 Hz, 1H), 7.77 (brs, 3H), 7.44 (d, J=8.4 Hz, 1H), 7.14 (s, 1H), 6.41-6.37 (m, 2H), 3.69 (s, 2H), 3.58-3.52 (m, 6H), 3.49 (s, 4H), 3.42-3.39 (m, 2H), 3.27-3.22 (m, 2H), 2.98-2.96 (m, 2H), 2.36 (s, 3H), 2.34 (s, 3H). MS (ESI) m/z=480.2 [M+H]⁺.

### Example 014. 4-((18-Amino-2-oxo-6,9,12,15-tetraoxa-3-azaoctadecyl)amino)-2-methyl-N-(5-methylthiazol-2-yl)benzamide (BL1-14)

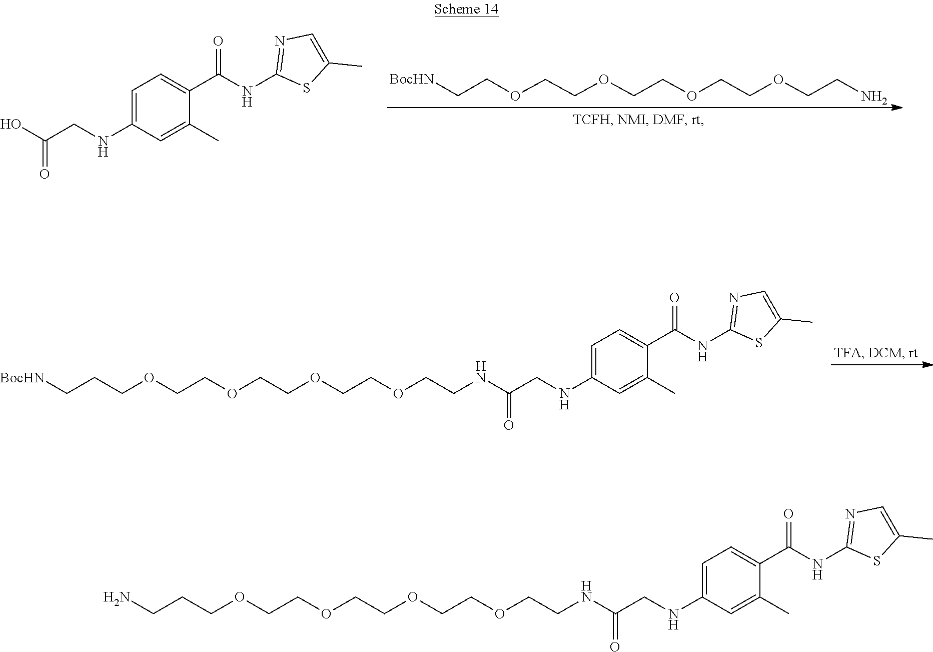 [SMILES: Cc1cnc(NC(=O)c2ccc(NCC(=O)O)cc2C)s1 ; CC(C)(C)OC(=O)NCCOCCOCCOCCOCCN ; Cc1cnc(NC(=O)c2ccc(NCC(=O)NCCOCCOCCOCCOCCCNC(=O)OC(C)(C)C)cc2C)s1 ; Cc1cnc(NC(=O)c2ccc(NCC(=O)NCCOCCOCCOCCOCCCN)cc2C)s1]

BL1-14 was synthesized following the standard procedures for preparing BL1-9 (320 mg, 55% yield over two steps). ¹HNMR (400 MHz, DMSO-d₆) δ 11.88 (brs, 1H), 7.96 (t, J=5.4 Hz, 1H), 7.75 (brs, 3H), 7.46-7.44 (d, 1H), 7.14 (d, J=1.2 Hz, 1H), 6.42-6.38 (m, 2H), 3.70 (s, 2H), 3.59-3.48 (m, 14H), 3.42-3.39 (m, 2H), 3.27-3.22 (m, 2H), 2.99-2.95 (m, 2H), 2.37 (s, 3H), 2.35 (s, 3H). MS (ESI) m/z=524.2 [M+H]⁺.

### Example 015. 4-((20-Amino-2-oxo-6,9,12,15,18-pentaoxa-3-azaicosyl)amino)-2-methyl-N-(5-methylthiazol-2-yl)benzamide (BL1-15)

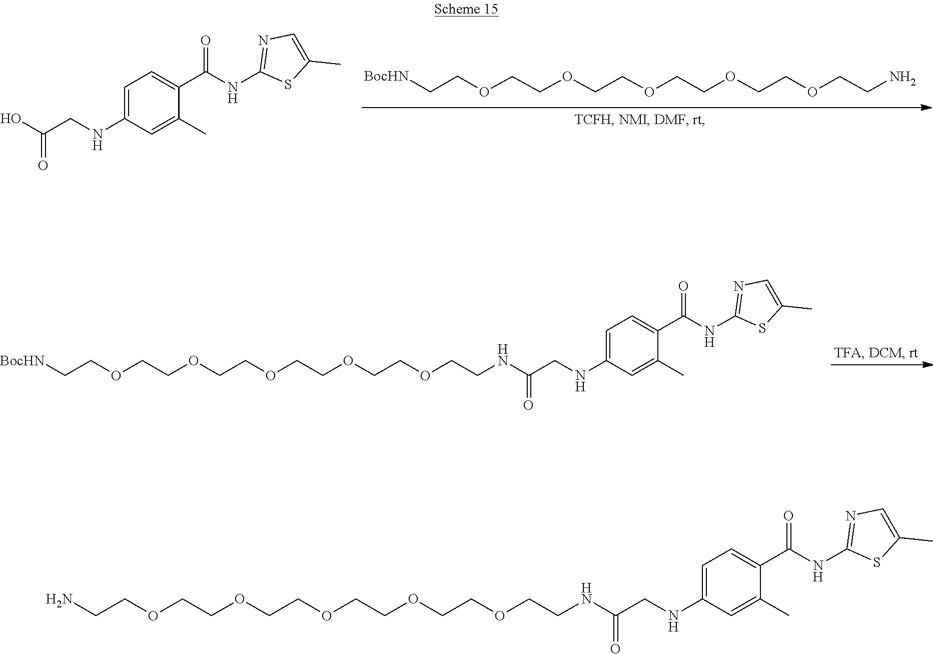 [SMILES: Cc1cnc(NC(=O)c2ccc(NCC(=O)O)cc2C)s1 ; CC(C)(C)OC(=O)NCCOCCOCCOCCOCCOCCN ; Cc1cnc(NC(=O)c2ccc(NCC(=O)NCCOCCOCCOCCOCCOCCNC(=O)OC(C)(C)C)cc2C)s1 ; Cc1cnc(NC(=O)c2ccc(NCC(=O)NCCOCCOCCOCCOCCOCCN)cc2C)s1]

BL1-15 was synthesized following the standard procedures for preparing BL1-9 (248 mg, 55% yield over two steps). ¹HNMR (400 MHz, DMSO-d₆) δ 11.86 (brs, 1H), 7.95 (t, J=5.4 Hz, 1H), 7.75 (brs, 3H), 7.46-7.44 (d, J=8.4 Hz, 1H), 7.14 (d, J=1.2 Hz, 1H), 6.42-6.38 (m, 2H), 3.69 (s, 2H), 3.59-3.48 (m, 18H), 3.42-3.39 (m, 2H), 3.26-3.22 (m, 2H), 2.99-2.95 (m, 2H), 2.36 (s, 3H), 2.34 (s, 3H). MS (ESI) m/z=568.3 [M+H]⁺.

### Example 016. 4-(2-((5-Aminopentyl)amino)-2-oxoethoxy)-2-methyl-N-(5-methylthiazol-2-yl)benzamide (BL1-16)

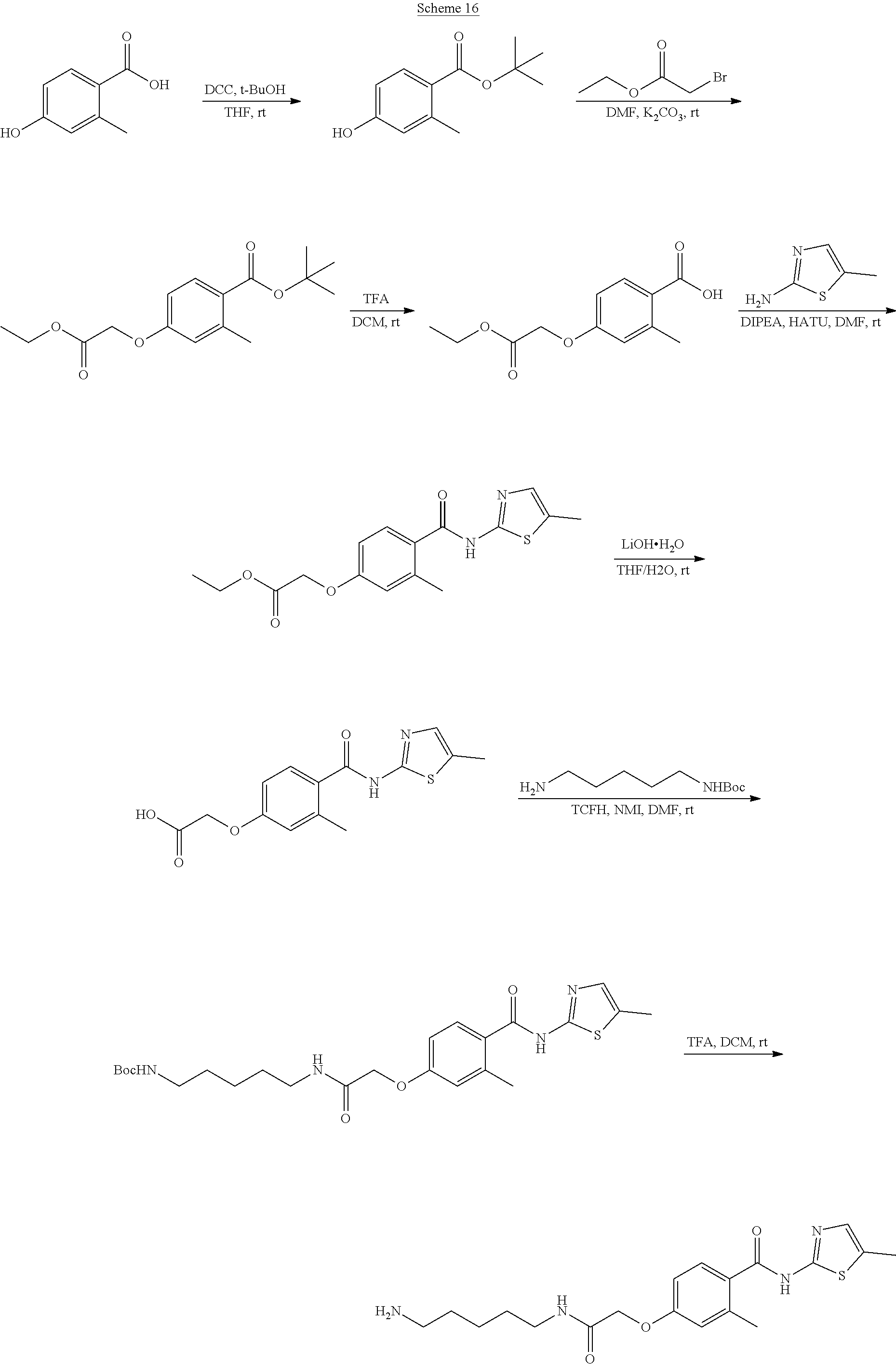

### Step 1. Synthesis of tert-butyl 4-hydroxy-2-methylbenzoate

To a solution of 4-hydroxy-2-methylbenzoic acid (3.00 g, 19.6 mmol) in THF (15 mL) and t-BuOH (15 mL) was added DCC (4.06 g, 19.6 mmol). The reaction mixture was stirred at rt for 12 h. Then the mixture was filtered and filtrate was concentrated under reduced pressure. The residue was purified by silica gel column chromatography (petroleum ether/EtOAc=5:1) to provide the title compound (1.5 g, 37% yield) as a yellow solid.

### Step 2. Synthesis of tert-butyl 4-(2-ethoxy-2-oxoethoxy)-2-methylbenzoate

To a solution of tert-butyl 4-hydroxy-2-methylbenzoate (1.50 g, 7.20 mmol) in DMF (10 mL) were added ethyl 2-bromoacetate (1.20 g, 7.20 mmol) and K₂CO₃ (1.20 g, 7.20 mmol). The reaction mixture was stirred at 25° C. for 12 h. Then the solution was poured into the water (50 mL) and extracted with EtOAc (3×50 mL). The combined organic phase was washed with brine, dried over Na₂SO₄, filtered, concentrated and purified by silica gel column chromatography (petroleum ether/EtOAc=5:1) to provide the title compound (1.00 g, 48% yield) as a white solid. MS (ESI) m/z=295.1 [M+H]⁺.

### Step 3. Synthesis of 4-(2-ethoxy-2-oxoethoxy)-2-methylbenzoic acid

To a solution of 2-ethoxy-6-methylbenzoic acid (1.0 g, 3.3 mmol) in DCM (10 mL) was added TFA (10 mL). The reaction mixture was stirred at rt for 2 h, before it was concentrated under vacuum to provide the title compound (950 mg, crude) as a yellow solid which was used for next step without further purification. MS (ESI) m/z=239.1 [M+H]⁺.

### Step 4. Synthesis of ethyl 2-(3-methyl-4-((5-methylthiazol-2-yl)carbamoyl)phenoxy)acetate

To a solution of 4-(2-ethoxy-2-oxoethoxy)-2-methylbenzoic acid (950 mg, crude) in DMF (10 mL) were added 5-methylthiazol-2-amine (910 mg, 8.00 mmol), HATU (1.52 g, 4.00 mmol) and DIPEA (1.00 g, 8.00 mmol). The reaction mixture was stirred at 25° C. for 16 h, before it was poured into water (50 mL) and extracted with EtOAc (3×50 mL). The combined organic phase was washed with brine, dried over Na₂SO₄, filtered and concentrated under vacuum. The residue was purified by prep-HPLC to provide the title compound (800 mg, 62% yield) as a white solid. MS (ESI) m/z=335.1 [M+H]⁺.

### Step 5. Synthesis of 2-(3-methyl-4-((5-methylthiazol-2-yl)carbamoyl)phenoxy)acetic acid

To a solution of ethyl 2-(3-methyl-4-((5-methylthiazol-2-yl)carbamoyl)phenoxy)acetate (900 mg, 2.69 mmol) in THF (5 mL) and H₂O (5 mL) was added LiOH·H₂O (220.09 mg, 5.39 mmol). The reaction mixture was stirred at 25° C. for 16 h, before it was concentrated under vacuum and acidified to pH=5 with 1N HCl. The solid was collected, washed with MeOH, and dried over vacuum to provide the title compound (760 mg, 92% yield) as a brown solid. ¹HNMR (400 MHz, DMSO-d₆) δ 12.17 (brs, 1H), 7.52 (d, J=8.4 Hz, 1H), 7.16 (s, 1H), 6.88 (s, 1H), 6.86-6.85 (m, 1H), 4.72 (s, 2H), 2.51 (s, 3H), 2.37 (s, 3H). MS (ESI) m/z=307.2 [M+H]⁺.

### Step 6. Synthesis of tert-butyl (5-(2-(3-methyl-4-((5-methylthiazol-2-yl)carbamoyl)phenoxy)acetamido)pentyl)carbamate

A solution of 2-(3-methyl-4-((5-methylthiazol-2-yl)carbamoyl)phenoxy)acetic acid (250 mg, 0.82 mmol), tert-butyl (5-aminopentyl)carbamate (266.4 mg, 1.23 mmol), TCFH (229.6 mg, 0.82 mmol) and N-methylimidazole (100.8 mg, 1.23 mmol) in DMF (10 mL) was stirred at rt for 16 h. The mixture was diluted with H₂O (50 mL) and extracted with EtOAc (3×50 mL). The combined organic phase was washed with brine (3×50 mL), dried over Na₂SO₄, filtered and concentrated under vacuum to provide the title compound as a brown solid (260 mg, crude), which was used for next step without further purification.

### Step 7. Synthesis of 4-(2-((5-aminopentyl)amino)-2-oxoethoxy)-2-methyl-N-(5-methylthiazol-2-yl)benzamide

To a solution of tert-butyl (7-(2-(3-methyl-4-((5-methylthiazol-2-yl)carbamoyl)phenoxy)acetamido)heptyl)carbamate (250 mg, crude) in DCM (10 mL) was added TFA (10 mL). The mixture was stirred at rt for 12 h, before it was concentrated and purified by prep-HPLC to provide the title compound as a brown solid (89.8 mg, 18% yield over two steps). ¹HNMR (400 MHz, DMSO-d₆) δ 12.17 (s, 1H), 8.13 (t, J=5.6 Hz, 1H), 7.73 (brs, 2H), 7.17 (d, J=1.6 Hz, 1H), 7.54 (d, J=8.4 Hz, 1H), 6.90 (d, J=2.4 Hz, 1H), 6.85 (dd, J=8.4, 2.4 Hz, 1H), 4.54 (s, 2H), 3.16-3.10 (m, 2H), 2.79-2.71 (m, 2H), 2.40 (s, 3H), 2.36 (s, 3H), 1.57-1.52 (m, 2H), 1.49-1.41 (m, 2H), 1.30-1.14 (m, 2H). MS (ESI) m/z=391.2 [M+H]⁺.

### Example 017. 4-(2-((7-Aminoheptyl)amino)-2-oxoethoxy)-2-methyl-N-(5-methylthiazol-2-yl)benzamide (BL1-17)

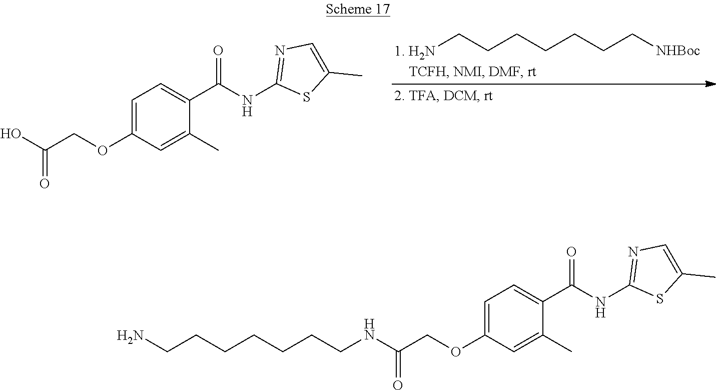

BL1-17 was synthesized following the standard procedures for preparing BL1-16 (26 mg, 8% yield over two steps). ¹HNMR (400 MHz, DMSO-d₆) δ 12.16 (brs, 1H), 8.10 (t, J=6.0 Hz, 1H), 7.60 (brs, 2H), 7.54 (d, J=8.4 Hz, 1H), 7.17 (d, J=1.6 Hz, 1H), 6.89 (d, J=2.0 Hz, 1H), 6.84 (dd, J=8.4, 2.4 Hz, 1H), 4.51 (s, 2H), 3.16-3.19 (m, 2H), 2.80-2.72 (m, 2H), 2.40 (s, 3H), 2.32 (s, 3H), 1.52-1.42 (m, 4H), 1.26-1.19 (m, 6H). MS (ESI) m/z=419.1 [M+H]⁺.

### Example 018. 4-(2-((9-Aminononyl)amino)-2-oxoethoxy)-2-methyl-N-(5-methylthiazol-2-yl)benzamide (BL1-18)

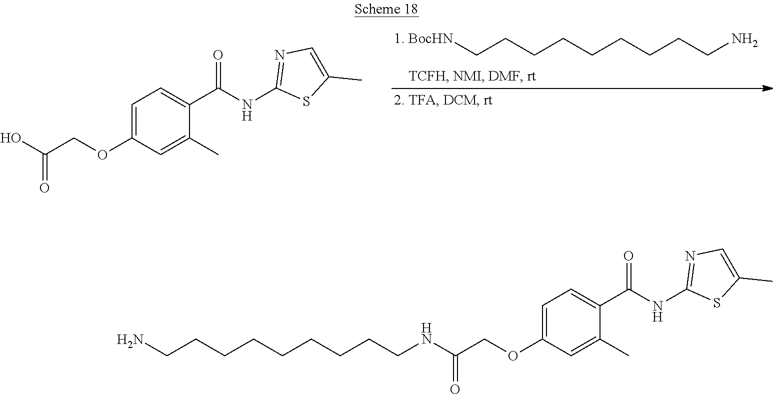

BL1-18 was synthesized following the standard procedures for preparing BL1-16 (90 mg, 40% yield over two steps). ¹HNMR (400 MHz, DMSO-d6) δ 12.20 (brs, 1H), 8.10 (t, J=6.0 Hz, 1H), 7.71 (brs, 2H), 7.58 (d, J=8.4 Hz, 1H), 7.17 (d, J=1.2 Hz, 1H), 6.89 (d, J=2.4 Hz, 1H), 6.84 (dd, J=8.4, 2.4 Hz, 1H), 4.52 (s, 2H), 3.14-3.10 (m, 2H), 3.10-3.08 (m, 2H), 2.73 (s, 3H), 2.71 (s, 3H), 1.52-1.43 (m, 4H), 1.16-1.14 (m, 10H). MS (ESI) m/z=447.5 [M+H]⁺.

### Example 019. 4-(2-((2-(2-(2-Aminoethoxy)ethoxy)ethyl)amino)-2-oxoethoxy)-2-methyl-N-(5-methylthiazol-2-yl)benzamide (BL1-19)

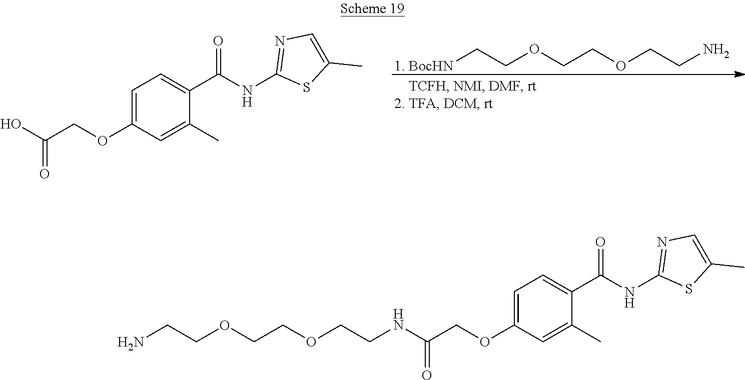

BL1-19 was synthesized following the standard procedures for preparing BL1-16 (50 mg, 23% yield over two steps). ¹HNMR (400 MHz, DMSO-d6) δ 12.20 (brs, 1H), 8.13 (t, J=6.0 Hz, 1H), 7.75 (brs, 2H), 7.55 (d, J=8.4 Hz, 1H), 7.18 (d, J=1.2 Hz, 1H), 6.90 (d, J=2.4 Hz, 1H), 6.85 (dd, J=8.4, 2.4 Hz, 1H), 4.54 (s, 2H), 3.47-3.44 (m, 6H), 3.38-3.36 (m, 2H), 3.32-2.28 (m, 2H), 3.30-2.96 (m, 2H), 2.40 (s, 3H), 2.37 (s, 3H). MS (ESI) m/z=437.2 [M+H]⁺.

### Example 020. 4-((14-Amino-2-oxo-6,9,12-trioxa-3-azatetradecyl)oxy)-2-methyl-N-(5-methylthiazol-2-yl)benzamide (BL1-20)

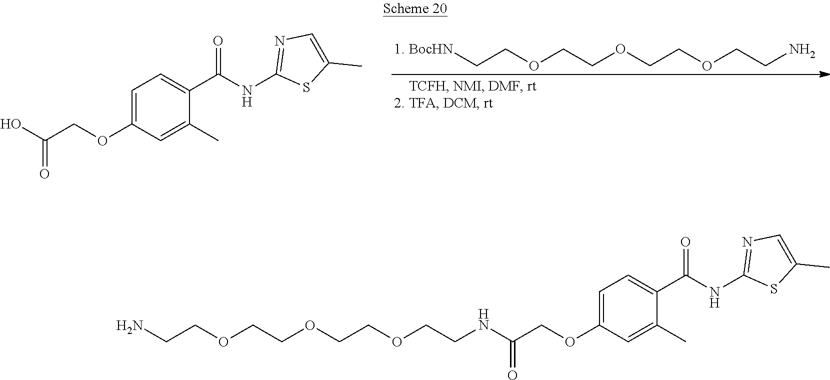

BL1-20 was synthesized following the standard procedures for preparing BL1-16 (138 mg, 57% yield over two steps). ¹HNMR (400 MHz, DMSO-d6) δ 12.18 (brs, 1H), 8.13 (t, J=5.6 Hz, 1H), 7.78 (brs, 2H), 7.56 (d, J=8.8 Hz, 1H), 7.17 (d, J=1.2 Hz, 1H), 6.90 (d, J=2.4 Hz, 1H), 6.85 (dd, J=8.4, 2.4 Hz, 1H), 4.55 (s, 2H), 3.59-3.55 (m, 10H), 3.52-3.49 (m, 2H), 3.32-2.29 (m, 2H), 3.30-2.96 (m, 2H), 2.40 (s, 3H), 2.37 (s, 3H). MS (ESI) m/z=481.2 [M+H]⁺.

### Example 021. 4-((17-Amino-2-oxo-6,9,12,15-tetraoxa-3-azaheptadecyl)oxy)-2-methyl-N-(5-methylthiazol-2-yl)benzamide (BL1-21)

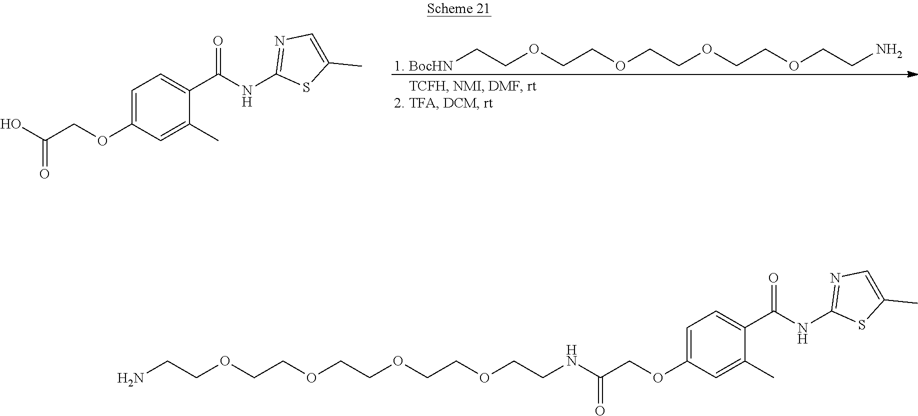

BL1-21 was synthesized following the standard procedures for preparing BL1-16 (61 mg, 28% yield over two steps). ¹HNMR (400 MHz, MeOD) δ 8.21 (s, 1H), 7.56 (d, J=8.8 Hz, 1H), 7.14 (s, 1H), 6.96 (s, 1H), 6.92 (d, J=8.4 Hz, 1H), 4.59 (s, 2H), 3.64-3.61 (m, 16H), 3.48-3.46 (m, 2H), 3.13-3.11 (m, 2H), 2.45 (s, 3H), 2.42 (s, 3H). MS (ESI) m/z=525.3 [M+H]⁺.

### Example 022. 4-((20-Amino-2-oxo-6,9,12,15,18-pentaoxa-3-azaicosyl)oxy)-2-methyl-N-(5-methylthiazol-2-yl)benzamide (BL1-22)

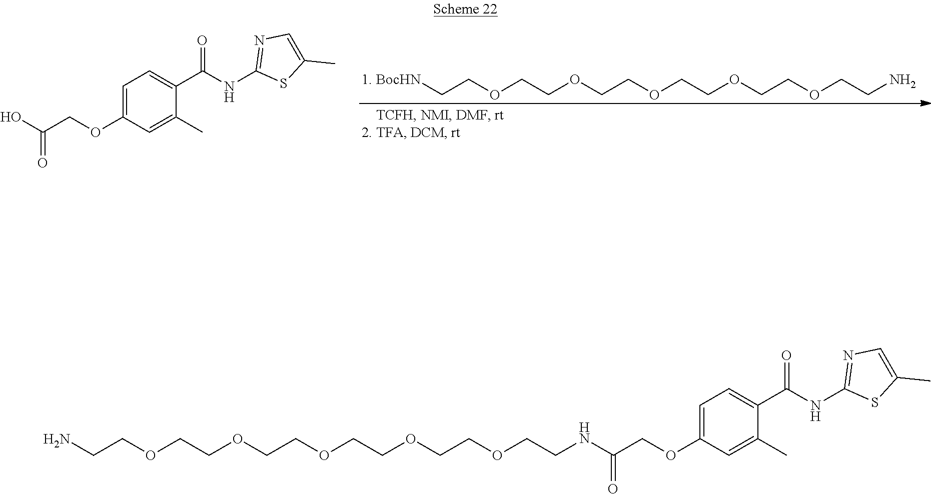

BL1-22 was synthesized following the standard procedures for preparing BL1-16 (98 mg, 35% yield over two steps). ¹HNMR (400 MHz, DMSO-d₆) δ 12.20 (brs, 1H), 8.13 (t, J=6.0 Hz, 1H), 7.75 (brs, 2H), 7.55 (d, J=8.4 Hz, 1H), 7.18 (d, J=1.2 Hz, 1H), 6.90 (d, J=2.4 Hz, 1H), 6.85 (dd, J=8.4, 2.4 Hz, 1H), 4.54 (s, 2H), 3.47-3.44 (m, 16H), 3.38-3.36 (m, 4H), 3.32-2.28 (m, 2H), 3.30-2.96 (m, 2H), 2.40 (s, 3H), 2.37 (s, 3H). MS (ESI) m/z=569.3 [M+H]⁺.

### Example 023. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-2-methyl-N-(5-phenylthiazol-2-yl)benzamide (CPD-001)

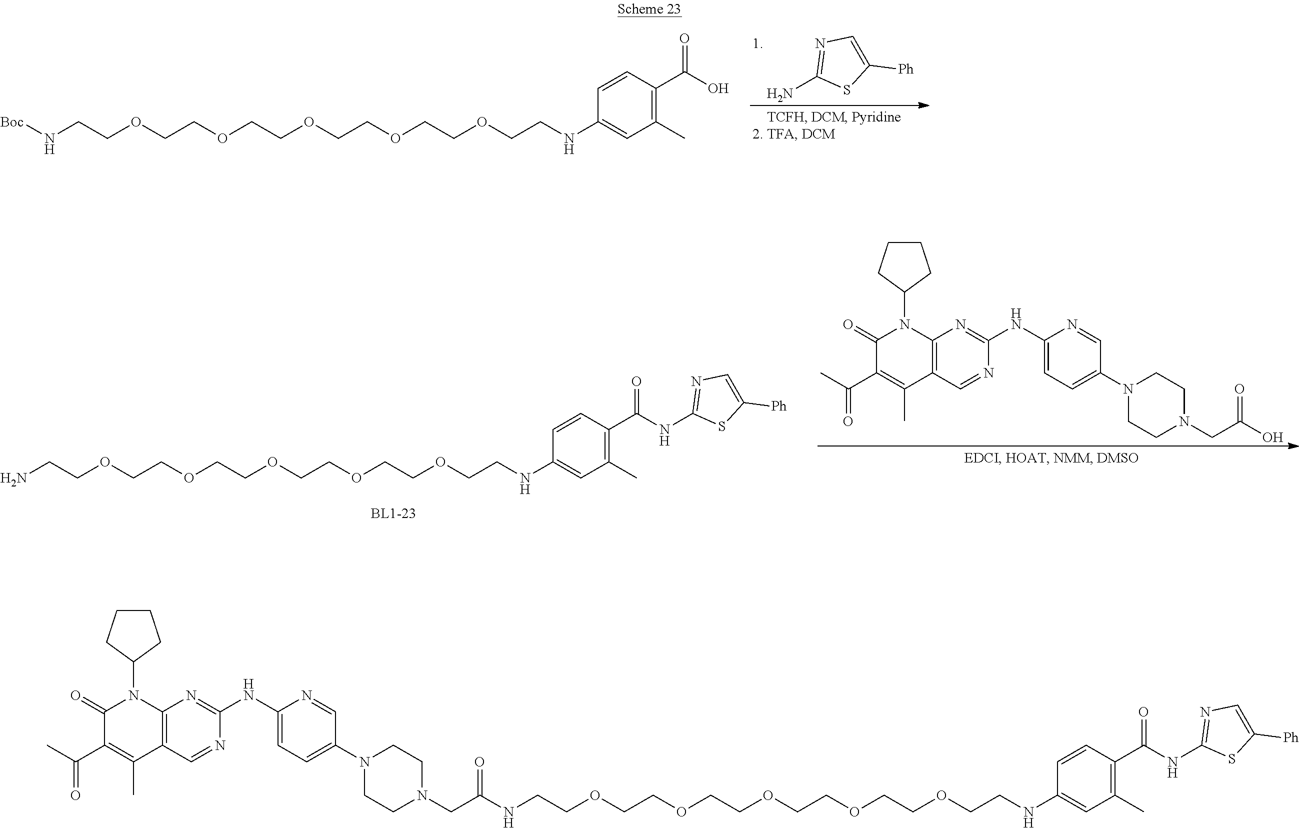 [SMILES: Cc1cc(NCCOCCOCCOCCOCCOCCNC(=O)OC(C)(C)C)ccc1C(=O)O ; Nc1ncc(-c2ccccc2)s1 ; Cc1cc(NCCOCCOCCOCCOCCOCCN)ccc1C(=O)Nc1ncc(-c2ccccc2)s1 ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)NCCOCCOCCOCCOCCOCCNc5ccc(C(=O)Nc6ncc(-c7ccccc7)s6)c(C)c5)CC4)cn3)nc2n(C2CCCC2)c1=O]

### Step 1. Synthesis of tert-butyl (17-((3-methyl-4-((5-phenylthiazol-2-yl)carbamoyl)phenyl)amino)-3,6,9,12,15-pentaoxaheptadecyl)carbamate

To a solution of 4-((2,2-dimethyl-4-oxo-3,8,11,14,17,20-hexaoxa-5-azadocosan-22-yl)amino)-2-methylbenzoic acid (10 mg, 0.02 mmol) in DCM (1 mL) was added a solution of TCFH (11 mg, 0.04 mmol) in DCM (1 mL). After the reaction was stirred at rt for 30 min, to the above mixture were added 5-phenylthiazol-2-amine (3.5 mg, 0.02 mmol) and pyridine (0.1 mL). The reaction mixture was stirred at rt for another 16 h, before it was diluted with DCM (5 mL), washed with 1N HCl (5 mL) and brine (5 mL). The organic layer was dried over anhydrous sodium sulfate, filtered and evaporated under reduced pressure. This residue was used directly in the next step without further purification. MS (ESI) m/z=673.4 [M+H]⁺.

### Step 2. Synthesis of 4-((17-amino-3,6,9,12,15-pentaoxaheptadecyl)amino)-2-methyl-N-(5-phenylthiazol-2-yl)benzamide

A mixture of tert-butyl (17-((3-methyl-4-((5-phenylthiazol-2-yl)carbamoyl)phenyl)amino)-3,6,9,12,15-pentaoxaheptadecyl)carbamate (10 mg, 0.17 mmol) in TFA (1 mL) and DCM (1 mL) was stirred at rt for 1 h. The resulting mixture was concentrated to provide the crude product as a light-yellow oil. This compound was used directly in the next step without further purification. MS (ESI) m/z=573.4 [M+H]⁺.

### Step 3. Synthesis of 4-((1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-2-methyl-N-(5-phenylthiazol-2-yl)benzamide

A solution of 4-((17-amino-3,6,9,12,15-pentaoxaheptadecyl)amino)-2-methyl-N-(5-phenylthiazol-2-yl)benzamide (10 mg, 0.02 mmol), 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid (10 mg, 0.02 mmol), EDCI (5.7 mg, 0.03 mmol), HOAt (4.1 mg, 0.03 mmol) and NMM (10.1 mg, 0.10 mmol) in DMSO (2 mL) was stirred at rt for 16 h. The reaction mixture was purified by reverse-phase chromatography to give the desired product (3.6 mg, 17% yield over 3 steps) as a yellow solid. MS (ESI) m/z=1060.6 [M+H]⁺.

### Example 024. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-2-methyl-N-(5-methylthiazol-2-yl)benzamide (CPD-002)

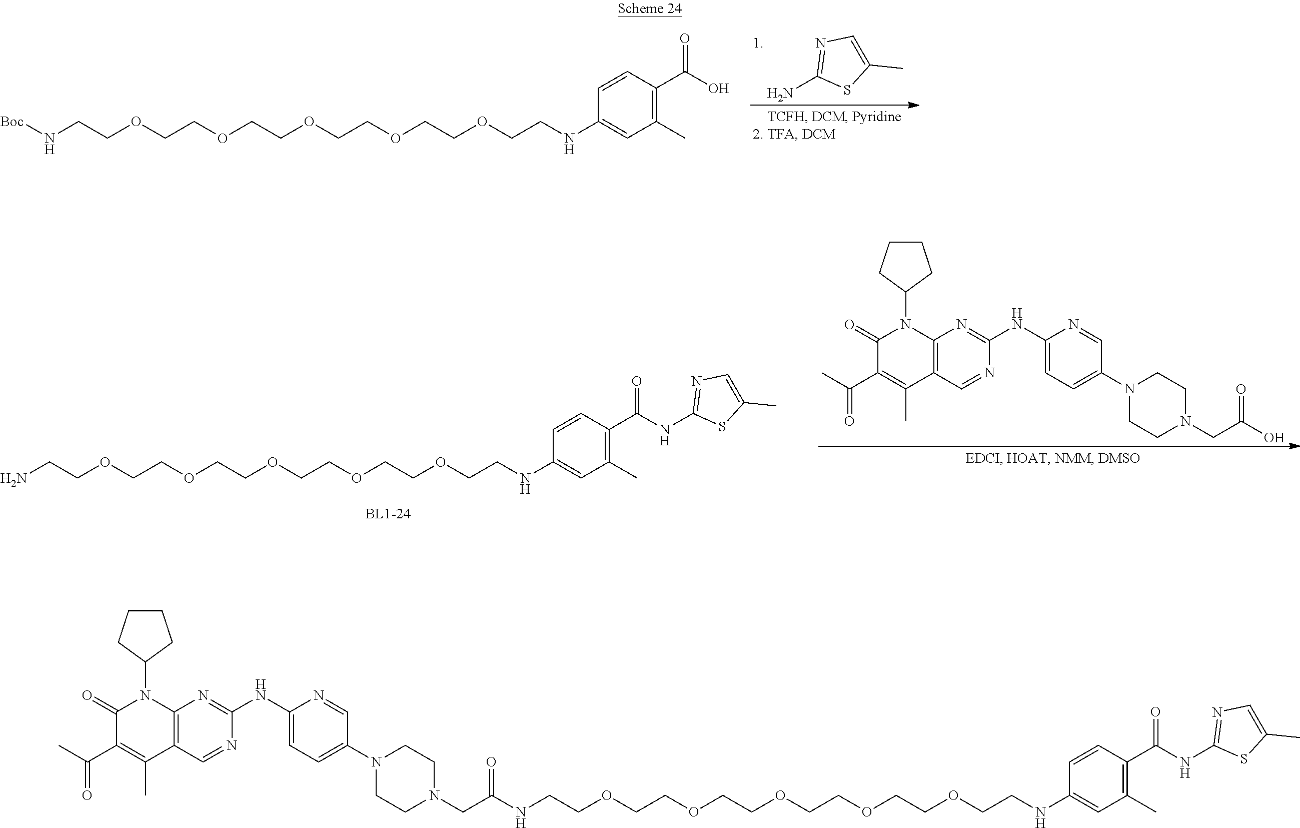

CPD-002 was synthesized following the standard procedures for preparing CPD-001 (3.3 mg, 17% yield over 3 steps). MS (ESI) m/z=998.5 [M+H]⁺.

### Example 025. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-2-methyl-N-(5-(trifluoromethyl)thiazol-2-yl)benzamide (CPD-003)

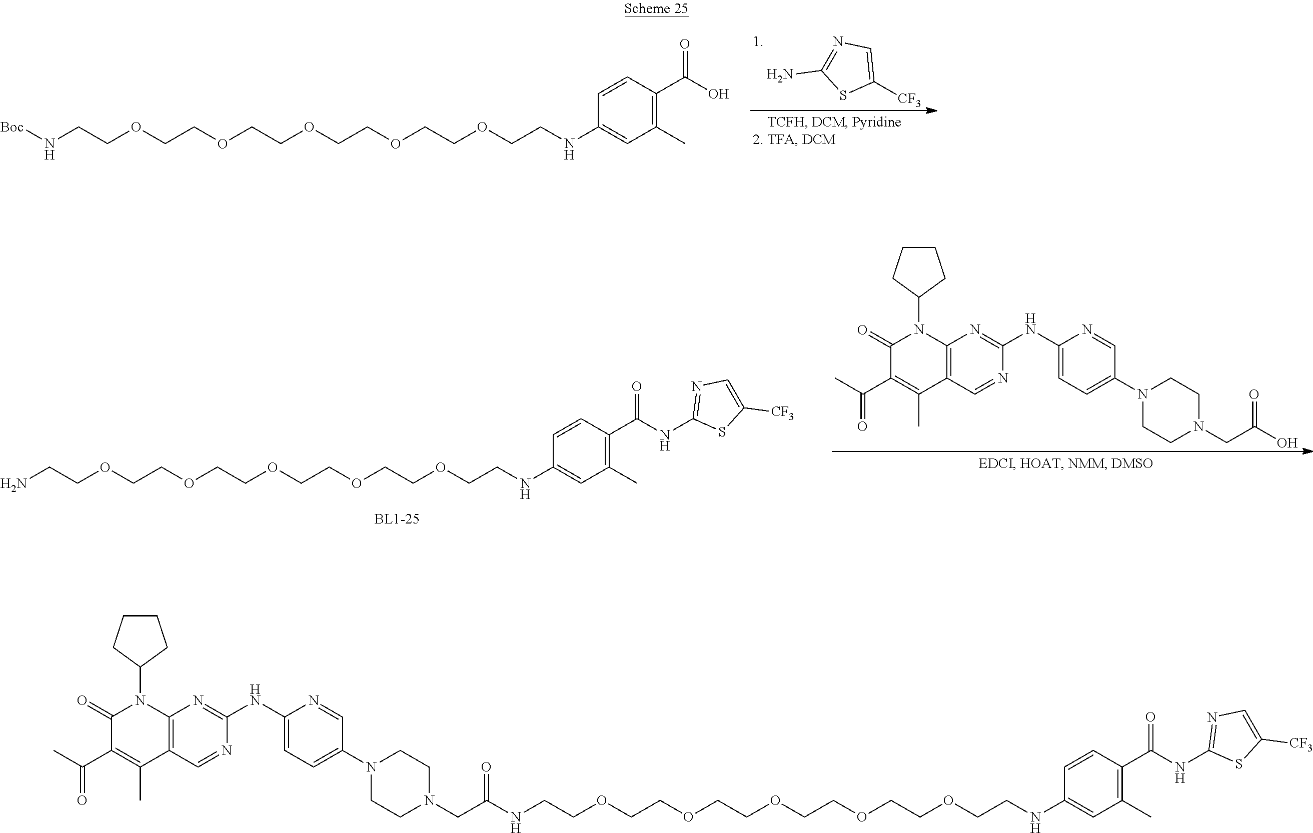 [SMILES: Cc1cc(NCCOCCOCCOCCOCCOCCNC(=O)OC(C)(C)C)ccc1C(=O)O ; Nc1ncc(C(F)(F)F)s1 ; Cc1cc(NCCOCCOCCOCCOCCOCCN)ccc1C(=O)Nc1ncc(C(F)(F)F)s1 ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)NCCOCCOCCOCCOCCOCCNc5ccc(C(=O)Nc6ncc(C(F)(F)F)s6)c(C)c5)CC4)cn3)nc2n(C2CCCC2)c1=O]

CPD-003 was synthesized following the standard procedures for preparing CPD-001 (1.6 mg, 8% yield over 3 steps). MS (ESI) m/z=1052.5 [M+H]⁺.

### Example 026. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-2-methyl-N-(thiazol-2-yl)benzamide (CPD-004)

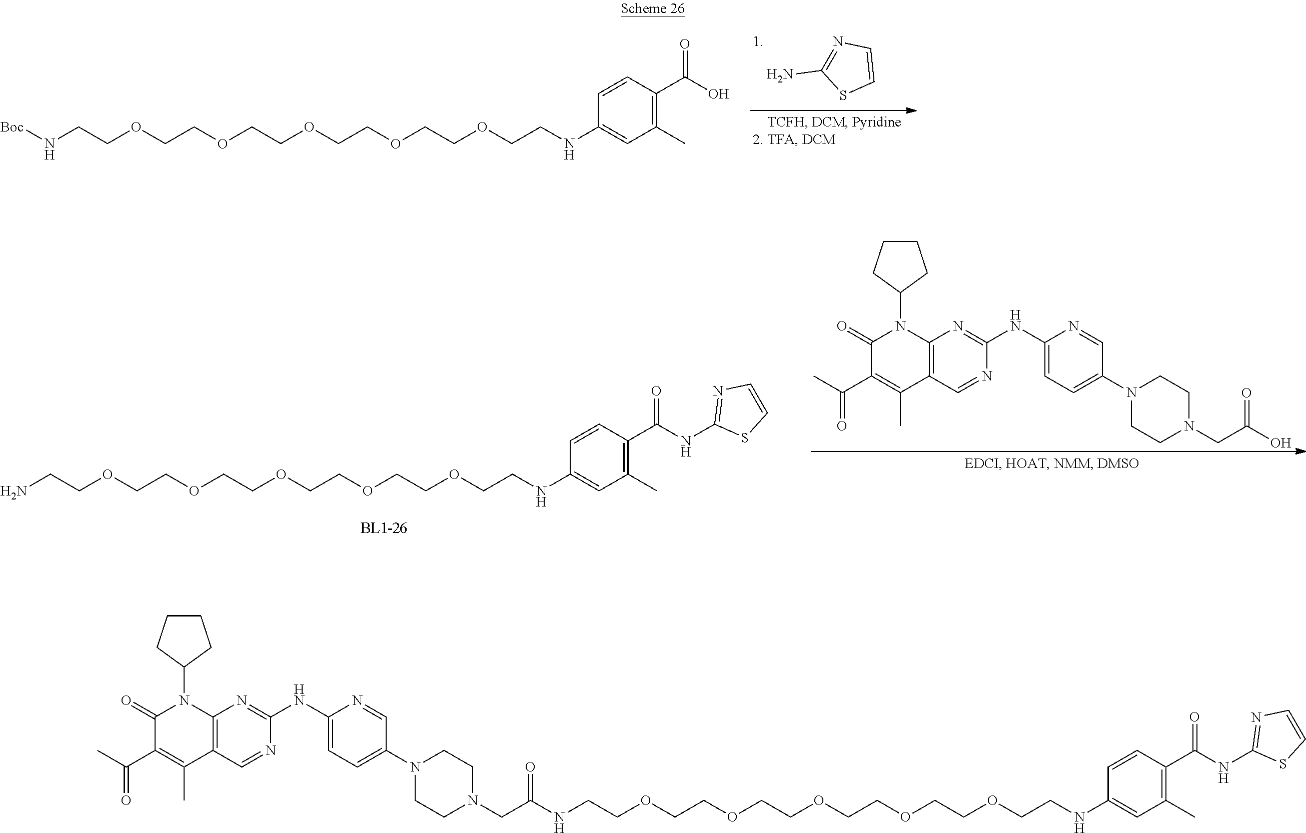 [SMILES: Cc1cc(NCCOCCOCCOCCOCCOCCNC(=O)OC(C)(C)C)ccc1C(=O)O ; Nc1nccs1 ; Cc1cc(NCCOCCOCCOCCOCCOCCN)ccc1C(=O)Nc1nccs1 ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)NCCOCCOCCOCCOCCOCCNc5ccc(C(=O)Nc6nccs6)c(C)c5)CC4)cn3)nc2n(C2CCCC2)c1=O]

CPD-004 was synthesized following the standard procedures for preparing CPD-001 (1.4 mg, 7% yield over 3 steps). MS (ESI) m/z=984.5 [M+H]⁺.

### Example 027. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-N-(5-chlorothiazol-2-yl)-2-methylbenzamide (CPD-005)

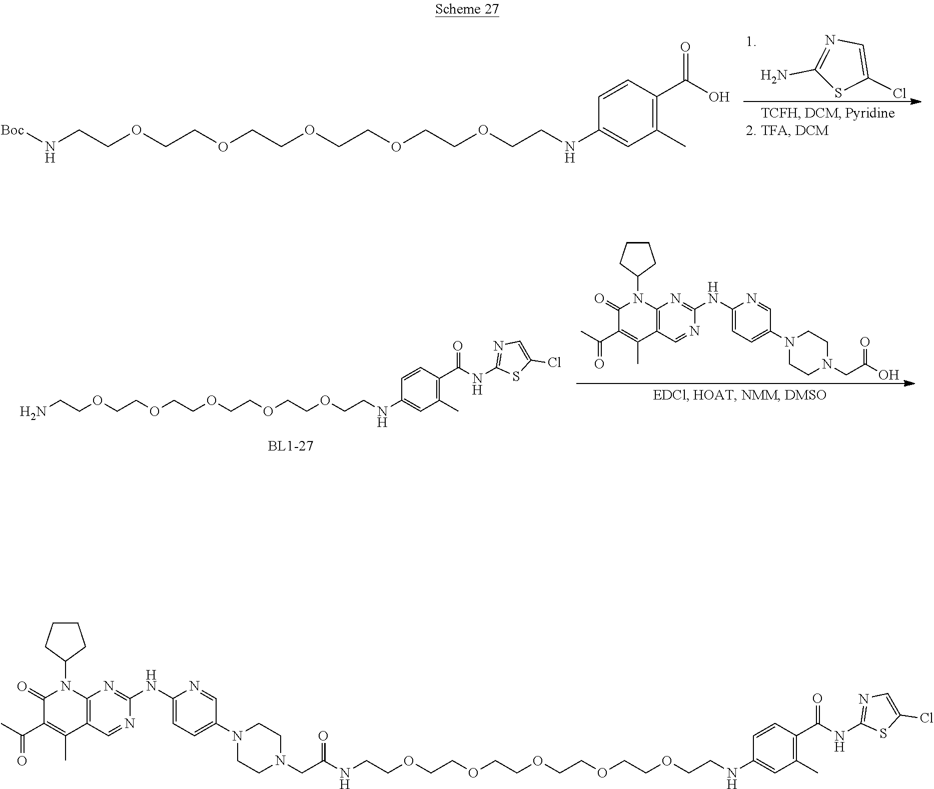 [SMILES: Cc1cc(NCCOCCOCCOCCOCCOCCNC(=O)OC(C)(C)C)ccc1C(=O)O ; Nc1ncc(Cl)s1 ; Cc1cc(NCCOCCOCCOCCOCCOCCN)ccc1C(=O)Nc1ncc(Cl)s1 ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)NCCOCCOCCOCCOCCOCCNc5ccc(C(=O)Nc6ncc(Cl)s6)c(C)c5)CC4)cn3)nc2n(C2CCCC2)c1=O]

CPD-005 was synthesized following the standard procedures for preparing CPD-001 (2.4 mg, 13% yield over 3 steps). MS (ESI) m/z=1018.5 [M+H]⁺.

### Example 028. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-N-(5-isopropylthiazol-2-yl)-2-methylbenzamide (CPD-006)

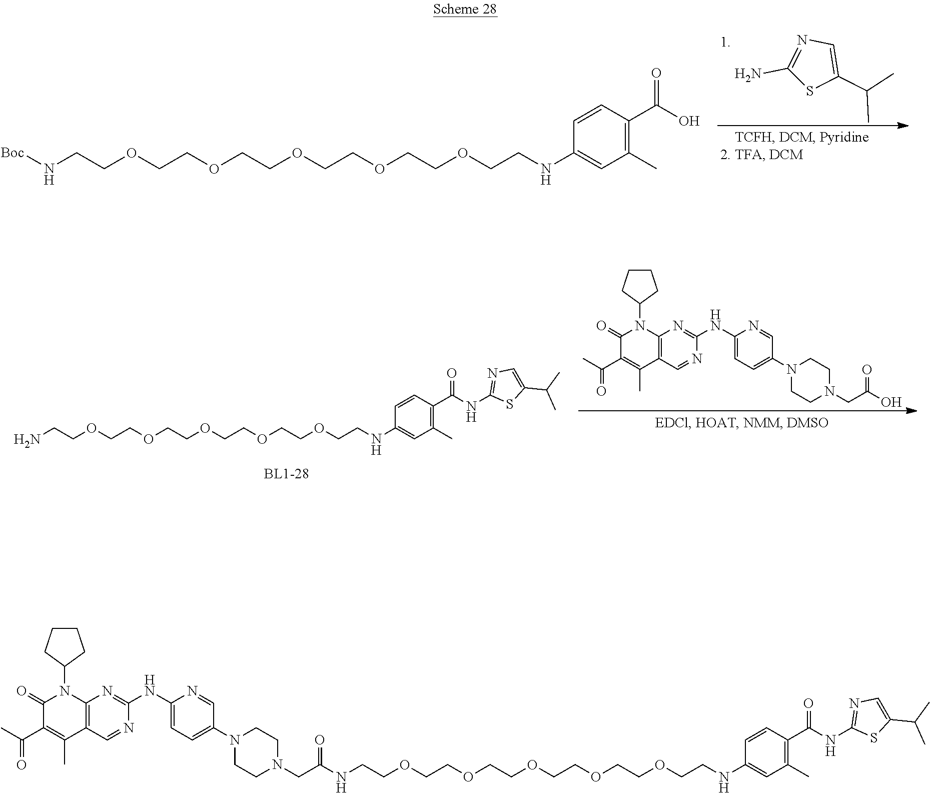

CPD-006 was synthesized following the standard procedures for preparing CPD-001 (7.5 mg, 26% yield over 3 steps). MS (ESI) m/z=1026.6 [M+H]⁺.

### Example 029. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-2-methyl-N-(4-phenylthiazol-2-yl)benzamide (CPD-007)

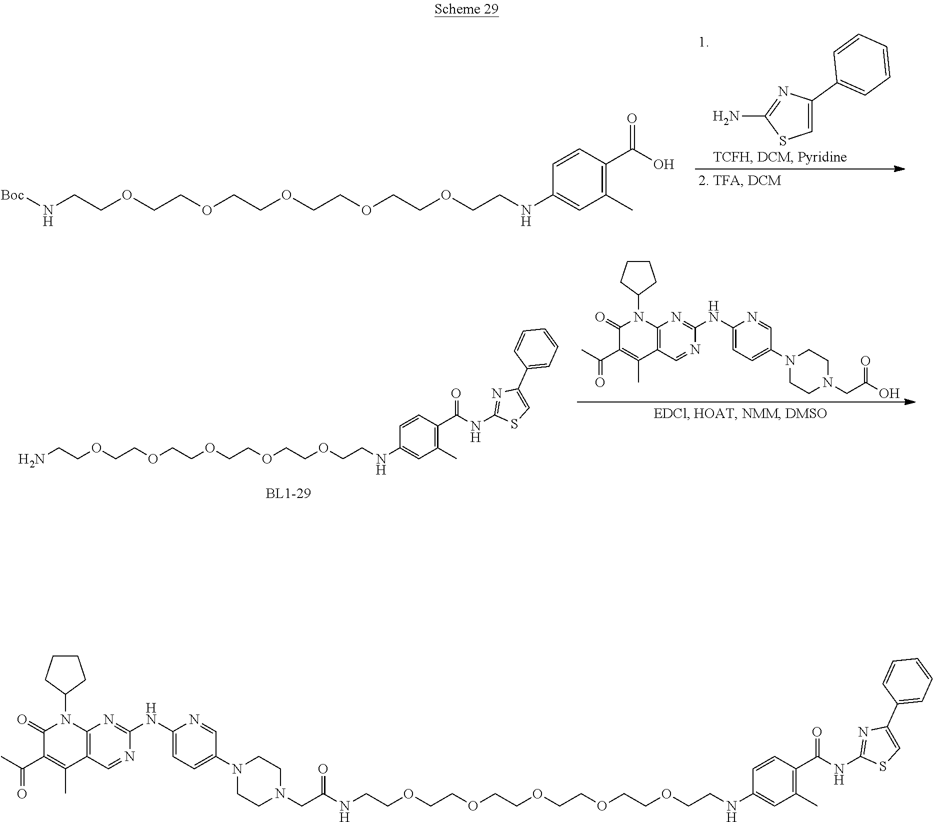

CPD-007 was synthesized following the standard procedures for preparing CPD-001 (6.3 mg, 21% yield over 3 steps). MS (ESI) m/z=1060.6 [M+H]⁺.

### Example 030. N⁴-(1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)-2-methyl-N¹-(5-methylthiazol-2-yl)terephthalamide (CPD-008)

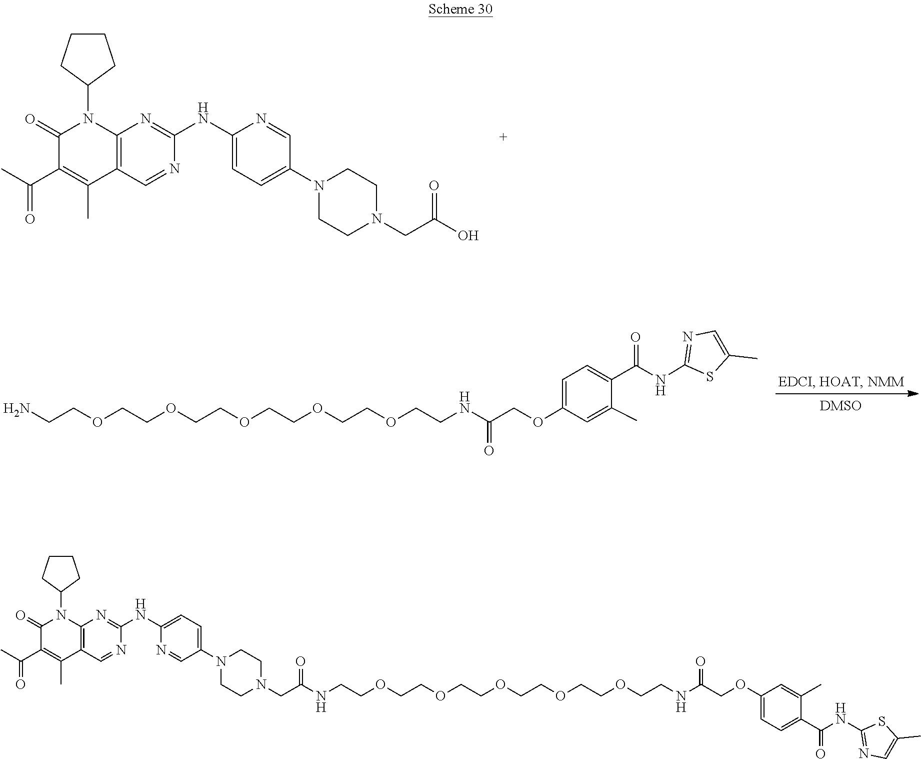

A solution of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid (5.0 mg, 0.009 mmol), N⁴-(17-amino-3,6,9,12,15-pentaoxaheptadecyl)-2-methyl-N¹-(5-methylthiazol-2-yl)terephthalamide (5.8 mg, 0.01 mmol), EDCI (2.9 mg, 0.015 mmol), HOAt (2.1 mg, 0.015 mmol) and NMM (10.1 mg, 0.10 mmol) in DMSO (2 mL) was stirred at rt for 16 h. The reaction mixture was poured into water (10 mL) and extracted with ethyl acetate (3×10 mL). The combined organic layers were washed with saturated brine (20 mL), dried over anhydrous sodium sulfate, filtered and evaporated under reduced pressure. The resulting residue was purified by reverse-phase chromatography to give the desired product (2.2 mg, 23% yield) as a yellow solid. MS (ESI) m/z=1026.5 [M+H]⁺.

### Example 031. N⁴-(5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)-2-methyl-N¹-(5-methylthiazol-2-yl)terephthalamide (CPD-009)

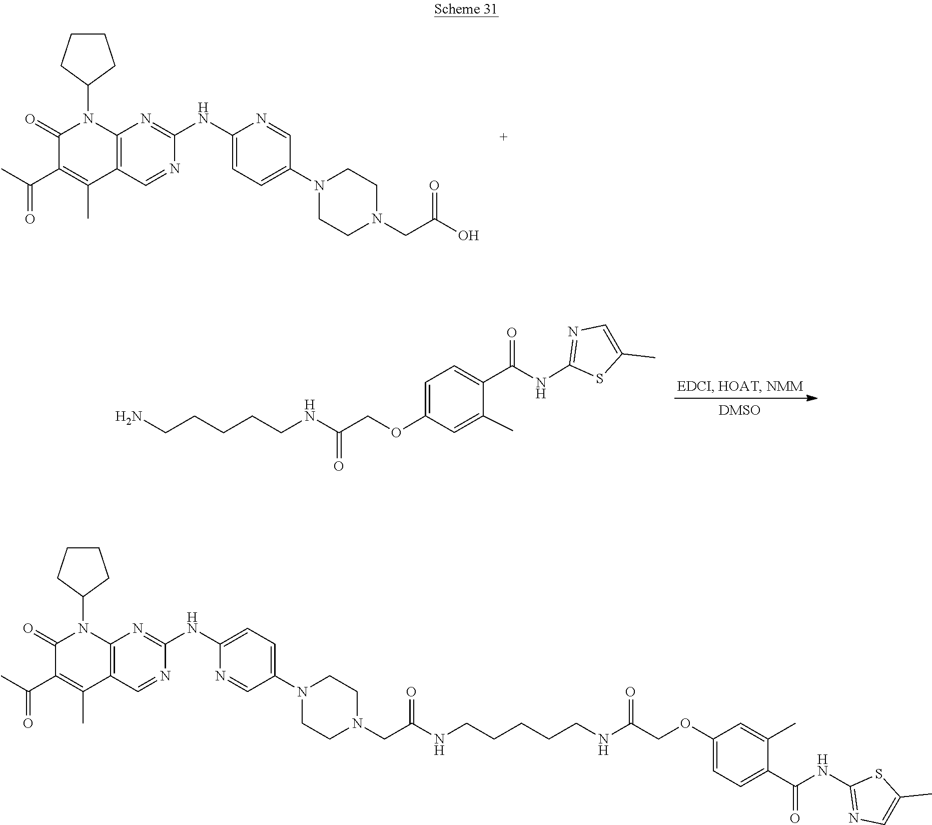 [SMILES: CC(=O)c1c(C)c2cnc(Nc3ccc(N4CCN(CC(=O)O)CC4)cn3)nc2n(C2CCCC2)c1=O ; "+" ; Cc1cnc(NC(=O)c2ccc(OCC(=O)NCCCCCN)cc2C)s1 ; CC(=O)c1c(C)c2cnc(Nc3ccc(N4CCN(CC(=O)NCCCCCNC(=O)COc5ccc(C(=O)Nc6ncc(C)s6)c(C)c5)CC4)cn3)nc2n(C2CCCC2)c1=O]

CPD-009 was synthesized following the standard procedure for preparing CPD-008 (4.7 mg, 56% yield). MS (ESI) m/z=848.5 [M+H]⁺.

### Example 032. N⁴-(2-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)ethyl)-2-methyl-N¹-(5-methylthiazol-2-yl)terephthalamide (CPD-010)

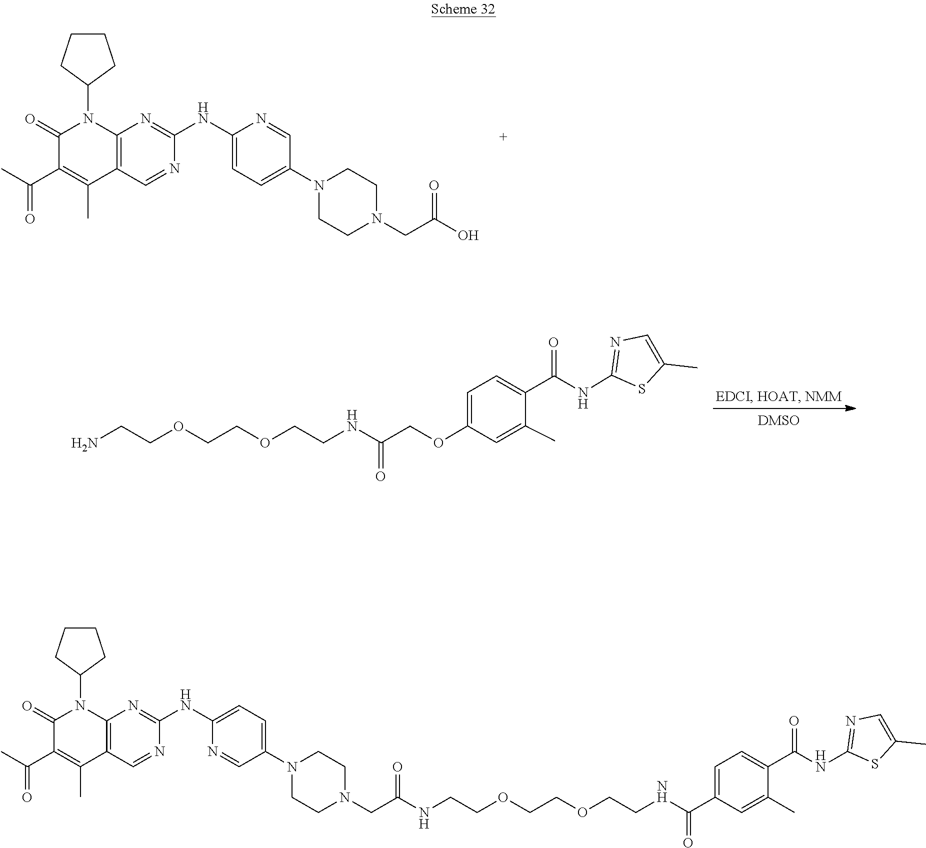

CPD-010 was synthesized following the standard procedure for preparing CPD-008 (3.4 mg, 39% yield). MS (ESI) m/z=894.4 [M+H]⁺.

### Example 033. N⁴-(1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12-trioxa-3-azatetradecan-14-yl)-2-methyl-N¹-(5-methylthiazol-2-yl)terephthalamide (CPD-011)

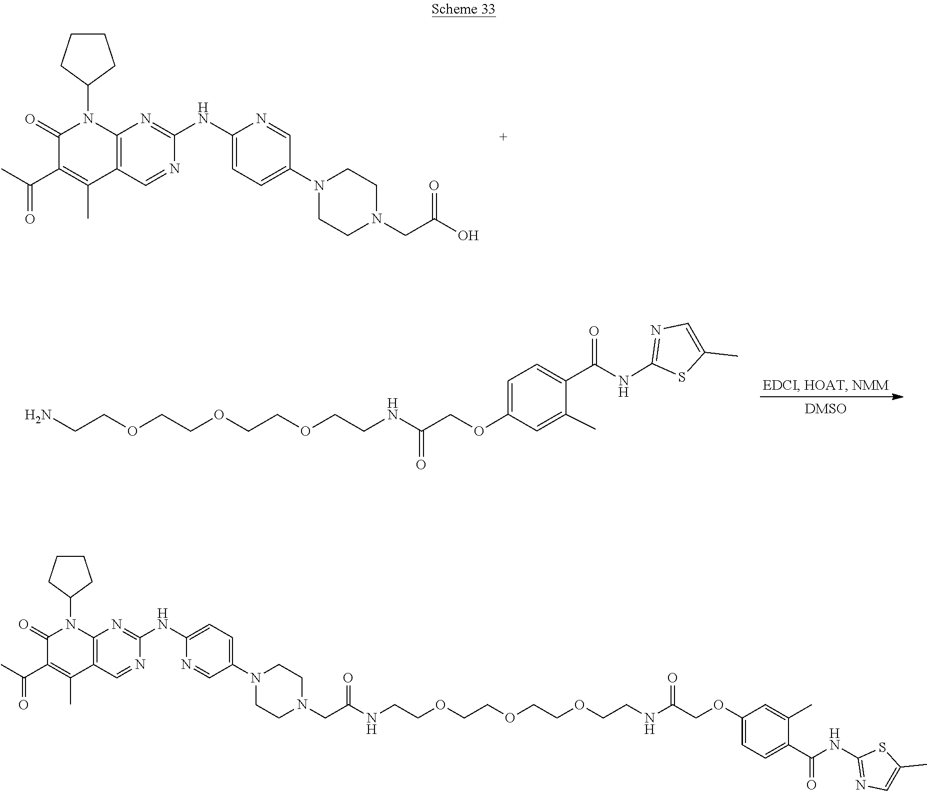

CPD-011 was synthesized following the standard procedure for preparing CPD-008 (5.1 mg, 55% yield). MS (ESI) m/z=938.5 [M+H]⁺.

### Example 034. N⁴-(1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15-tetraoxa-3-azaheptadecan-17-yl)-2-methyl-N¹-(5-methylthiazol-2-yl)terephthalamide (CPD-012)

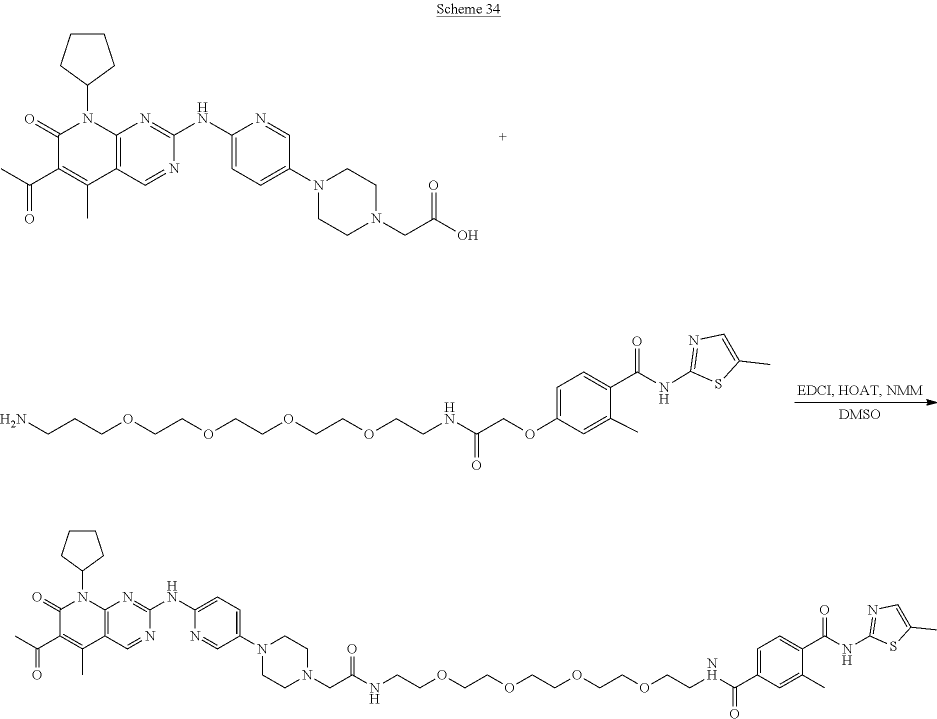

CPD-012 was synthesized following the standard procedure for preparing CPD-008 (5.8 mg, 60% yield). MS (ESI) m/z=982.5 [M+H]⁺.

### Example 035. 4-((20-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2,19-dioxo-6,9,12,15-tetraoxa-3,18-diazaicosyl)amino)-2-methyl-N-(5-methylthiazol-2-yl)benzamide (CPD-013)

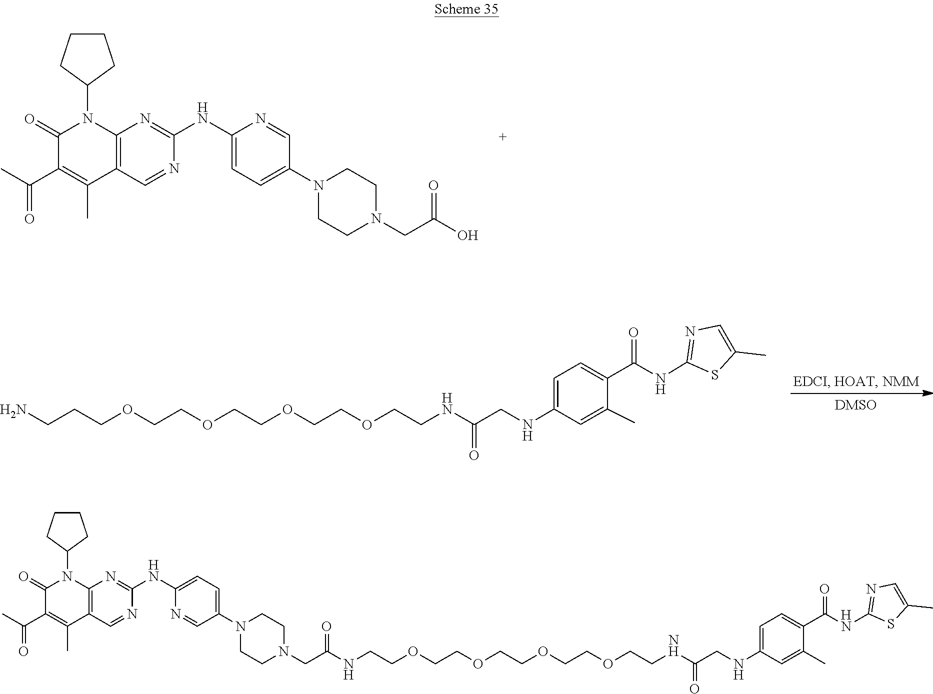

CPD-013 was synthesized following the standard procedure for preparing CPD-008 (4.8 mg, 48% yield). MS (ESI) m/z=1011.5 [M+H]⁺.

### Example 036. 4-((23-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2,22-dioxo-6,9,12,15,18-pentaoxa-3,21-diazatricosyl)amino)-2-methyl-N-(5-methylthiazol-2-yl)benzamide (CPD-014)

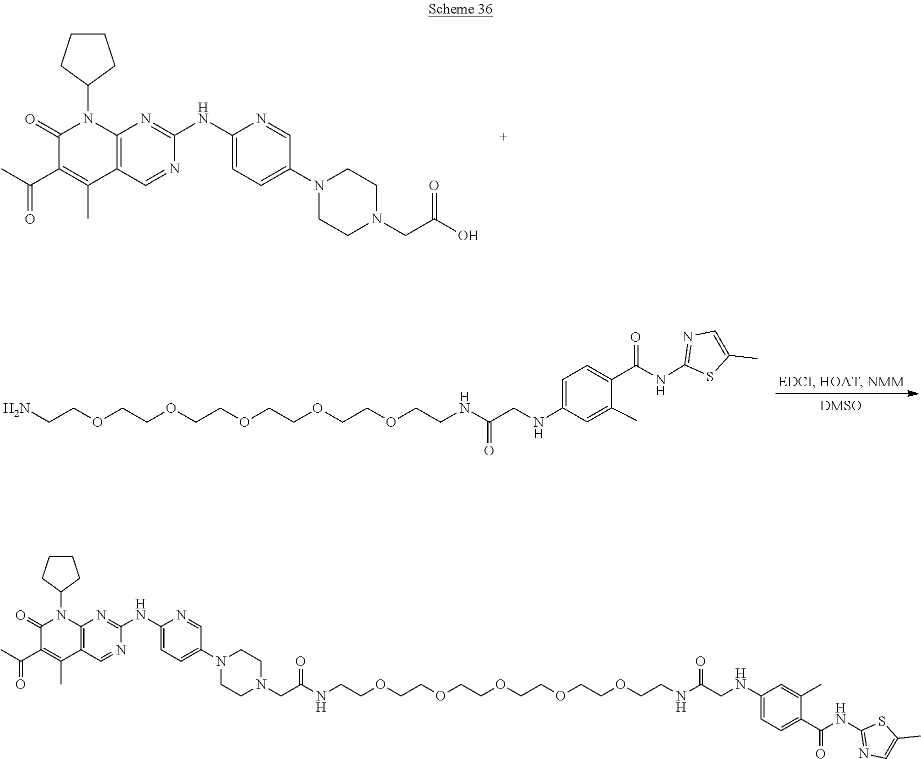

CPD-014 was synthesized following the standard procedure for preparing CPD-008 (4.4 mg, 44% yield). MS (ESI) m/z=1055.6 [M+H]⁺.

### Example 037. 4-((2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-2-oxoethyl)amino)-2-methyl-N-(5-methylthiazol-2-yl)benzamide (CPD-015)

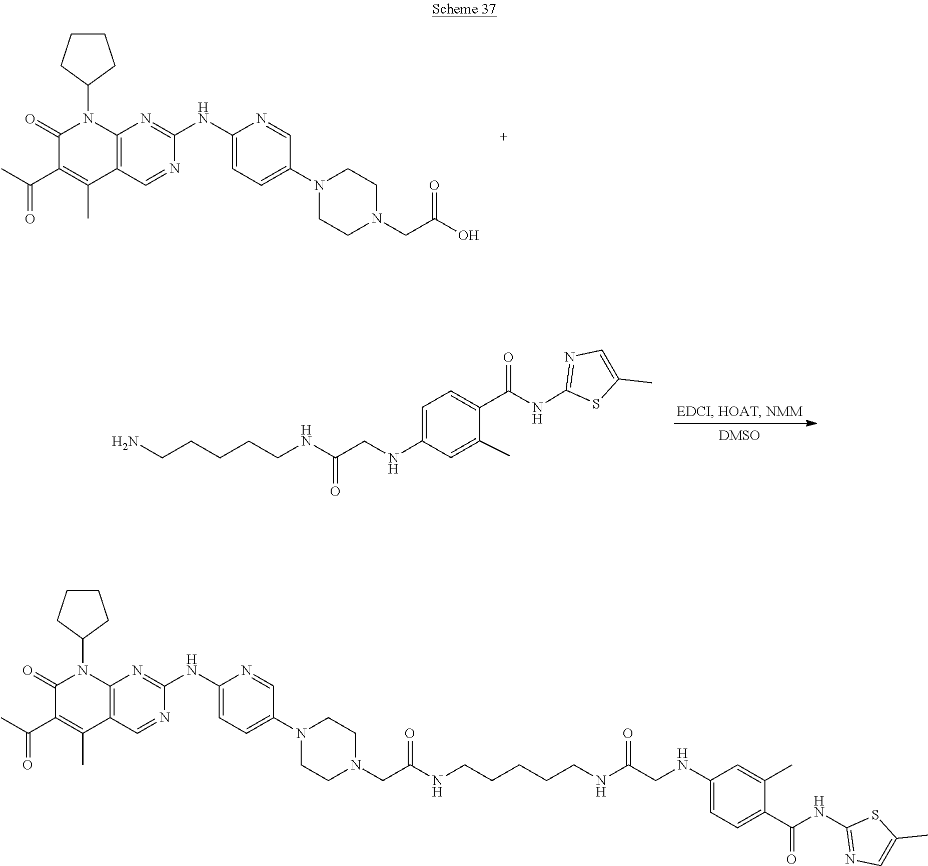 [SMILES: CC(=O)c1c(C)c2cnc(Nc3ccc(N4CCN(CC(=O)O)CC4)cn3)nc2n(C2CCCC2)c1=O ; "+" ; Cc1cnc(NC(=O)c2ccc(NCC(=O)NCCCCCN)cc2C)s1 ; CC(=O)c1c(C)c2cnc(Nc3ccc(N4CCN(CC(=O)NCCCCCNC(=O)CNc5ccc(C(=O)Nc6ncc(C)s6)c(C)c5)CC4)cn3)nc2n(C2CCCC2)c1=O]

CPD-015 was synthesized following the standard procedure for preparing CPD-008 (4.5 mg, 52% yield). MS (ESI) m/z=877.5 [M+H]⁺.

### Example 038. 4-((2-((7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-2-oxoethyl)amino)-2-methyl-N-(5-methylthiazol-2-yl)benzamide (CPD-016)

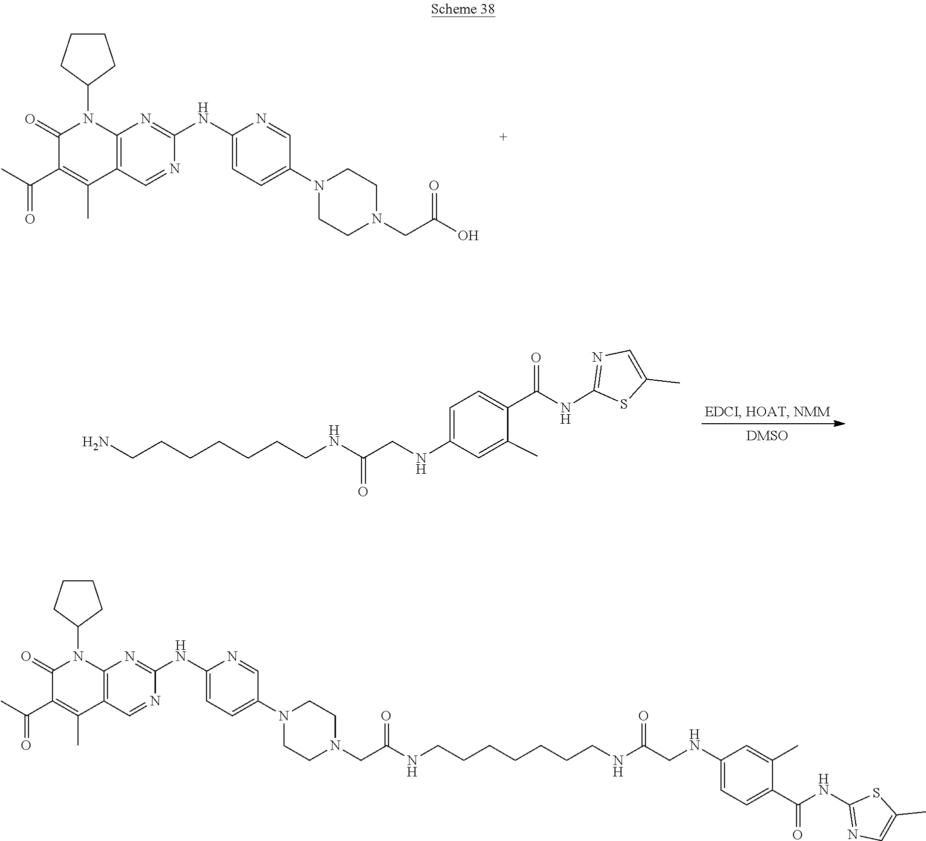

CPD-016 was synthesized following the standard procedure for preparing CPD-008 (4.4 mg, 49% yield). MS (ESI) m/z=905.5 [M+H]⁺.

### Example 039. 4-((14-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2,13-dioxo-6,9-dioxa-3,12-diazatetradecyl)amino)-2-methyl-N-(5-methylthiazol-2-yl)benzamide (CPD-017)

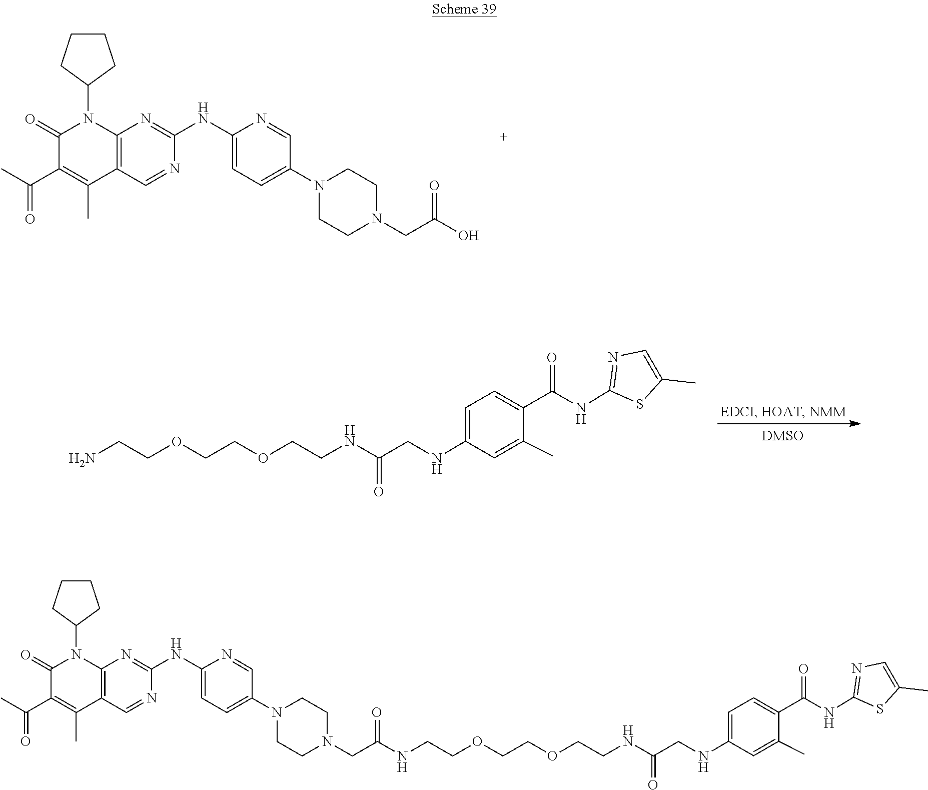

CPD-017 was synthesized following the standard procedure for preparing CPD-008 (3.7 mg, 41% yield). MS (ESI) m/z=923.6 [M+H]⁺.

### Example 040. 4-((17-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2,16-dioxo-6,9,12-trioxa-3,15-diazaheptadecyl)amino)-2-methyl-N-(5-methylthiazol-2-yl)benzamide (CPD-018)

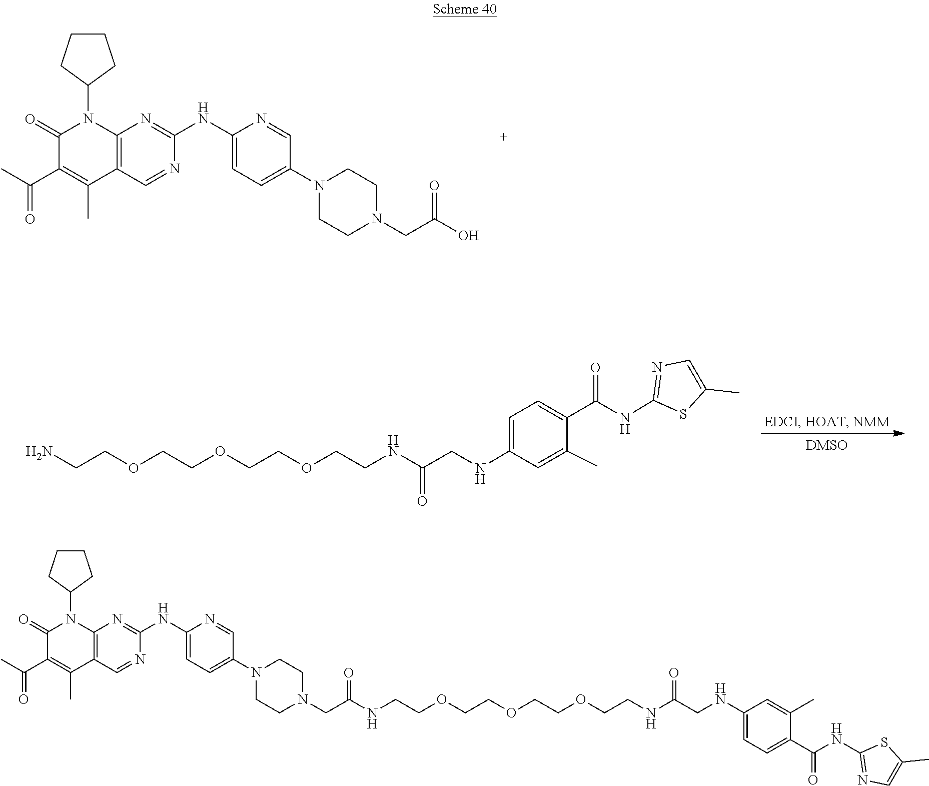

CPD-018 was synthesized following the standard procedure for preparing CPD-008 (5.5 mg, 58% yield). MS (ESI) m/z=967.5 [M+H]⁺.

### Example 041. 4-((23-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2,22-dioxo-6,9,12,15,18-pentaoxa-3,21-diazatricosyl)oxy)-2-methyl-N-(5-methylthiazol-2-yl)benzamide (CPD-019)

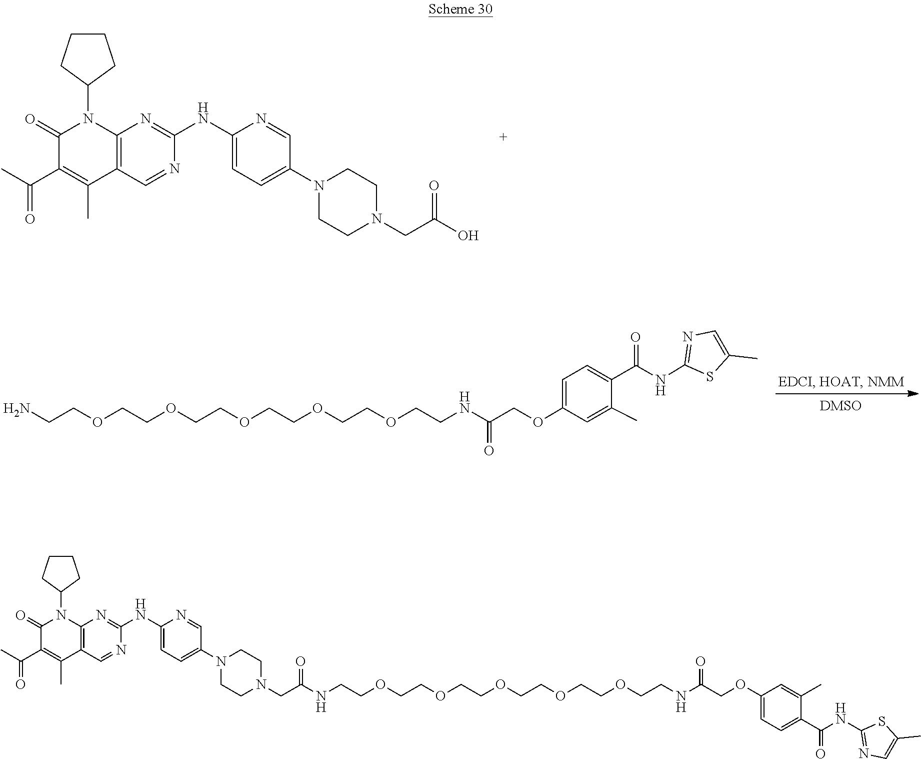

CPD-019 was synthesized following the standard procedure for preparing CPD-008 (4.6 mg, 44% yield). MS (ESI) m/z=1056.6 [M+H]⁺.

### Example 042. 4-((1-(4-((6-((5-Fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxo-6,9,12,15,18-pentaoxa-3-azaicosan-20-yl)amino)-2-methyl-N-(5-methylthiazol-2-yl)benzamide (CPD-020)

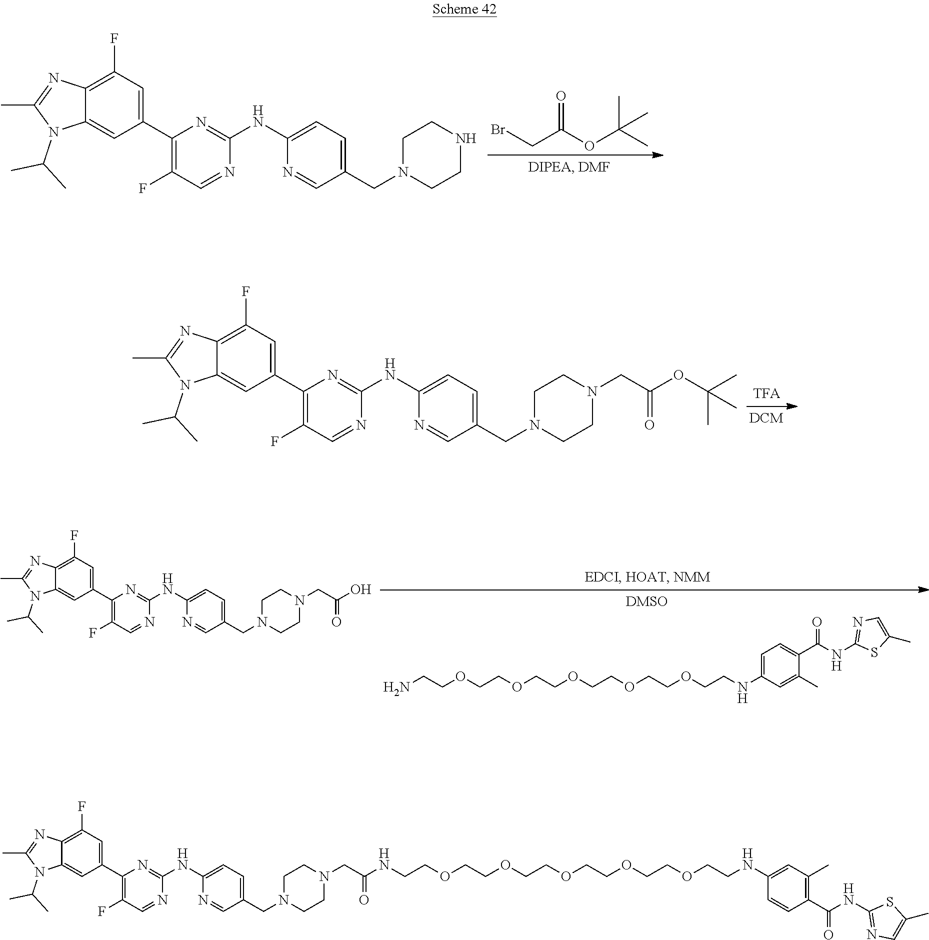

### Step 1. Synthesis of tert-butyl 2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetate

To a solution of 5-fluoro-4-(4-fluoro-1-isopropyl-2-methyl-1H-benzo[d]imidazol-6-yl)-N-(5-(piperazin-1-ylmethyl)pyridin-2-yl)pyrimidin-2-amine (50 mg, 0.1 mmol) and tert-butyl 2-bromoacetate (23 mg, 0.12 mmol) in DMF (2 mL) was added DIPEA (39 mg, 0.3 mmol) at rt. The reaction mixture was stirred at rt for 4 h, before it was purified by reverse-phase chromatography to provide the desired product (45 mg, 76% yield) as a white solid. MS (ESI) m/z=593.4 [M+H]⁺.

### Step 2. Synthesis of 2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetic acid

A mixture of tert-butyl 2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetate (45 mg, 0.07 mmol) in TFA (1 mL) and DCM (1 mL) was stirred at rt for 1 h. The resulting mixture was concentrated and purified by reverse-phase chromatography to provide the desired product (35 mg, 95% yield) as a white solid. MS (ESI) m/z=537.3 [M+H]⁺.

### Step 3. Synthesis of 4-((1-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxo-6,9,12,15,18-pentaoxa-3-azaicosan-20-yl)amino)-2-methyl-N-(5-methylthiazol-2-yl)benzamide

A solution of 2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetic acid (5.0 mg, 0.009 mmol), 4-((17-amino-3,6,9,12,15-pentaoxaheptadecyl)amino)-2-methyl-N-(5-methylthiazol-2-yl)benzamide (5.1 mg, 0.01 mmol), EDCI (2.9 mg, 0.015 mmol), HOAt (2.1 mg, 0.015 mmol) and NMM (10.1 mg, 0.10 mmol) in DMSO (1.5 mL) was stirred at rt for 16 h. The reaction mixture was poured into water (10 mL) and extracted with ethyl acetate (3×10 mL). The combined organic layers were washed with saturated brine (10 mL), dried over anhydrous sodium sulfate, filtered and evaporated under reduced pressure. The resulting residue was purified by reverse-phase chromatography and prep-TLC to give the desired product (1.8 mg, 20% yield) as a white solid. MS (ESI) m/z=1029.6 [M+H]⁺.

### Example 043. 4-((1-(4-(6-((5-Fluoro-4-(4-fluoro-1-isopropyl-2-methyl-1H-benzo[d]imidazol-6-yl)pyrimidin-2-yl)amino)pyridin-3-yl)piperazin-1-yl)-2-oxo-6,9,12,15,18-pentaoxa-3-azaicosan-20-yl)amino)-2-methyl-N-(5-methylthiazol-2-yl)benzamide (CPD-021)

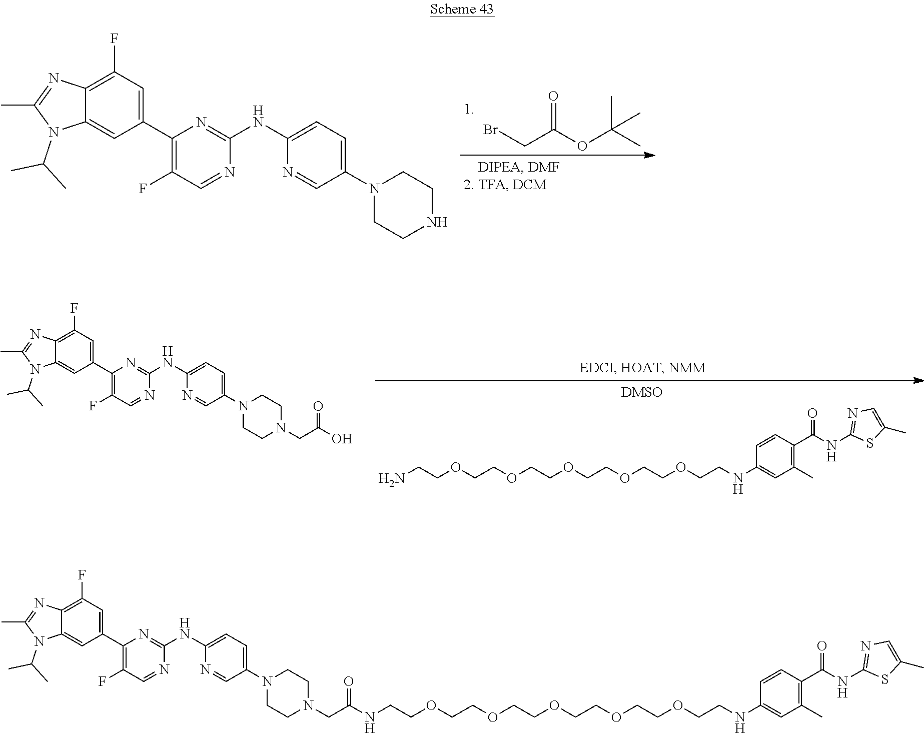

CPD-021 was synthesized following the standard procedures for preparing CPD-020 (2.5 mg, 26% yield over 3 steps). MS (ESI) m/z=1015.5 [M+H]⁺.

### Example 044. 2-Methyl-N-(5-methylthiazol-2-yl)-4-((2-oxo-1-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)-6,9,12,15,18-pentaoxa-3-azaicosan-20-yl)amino)benzamide (CPD-022)

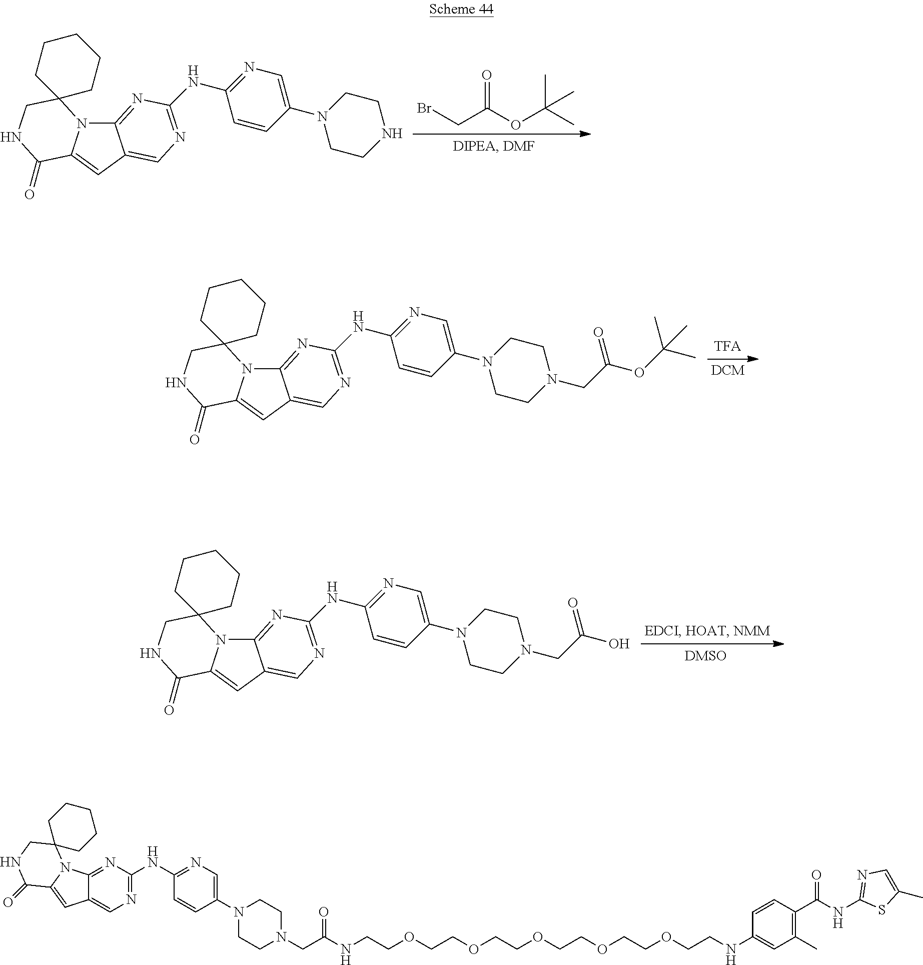

CPD-022 was synthesized following the standard procedures for preparing CPD-020 (2.1 mg, 21% yield over 3 steps). MS (ESI) m/z=983.5 [M+H]⁺.

### Example 045. 7-Cyclopentyl-N,N-dimethyl-2-((5-(4-(20-((3-methyl-4-((5-methylthiazol-2-yl)carbamoyl)phenyl)amino)-2-oxo-6,9,12,15,18-pentaoxa-3-azaicosyl)piperazin-1-yl)pyridin-2-yl)amino)-7H-pyrrolo[2,3-d]pyrimidine-6-carboxamide (CPD-023)

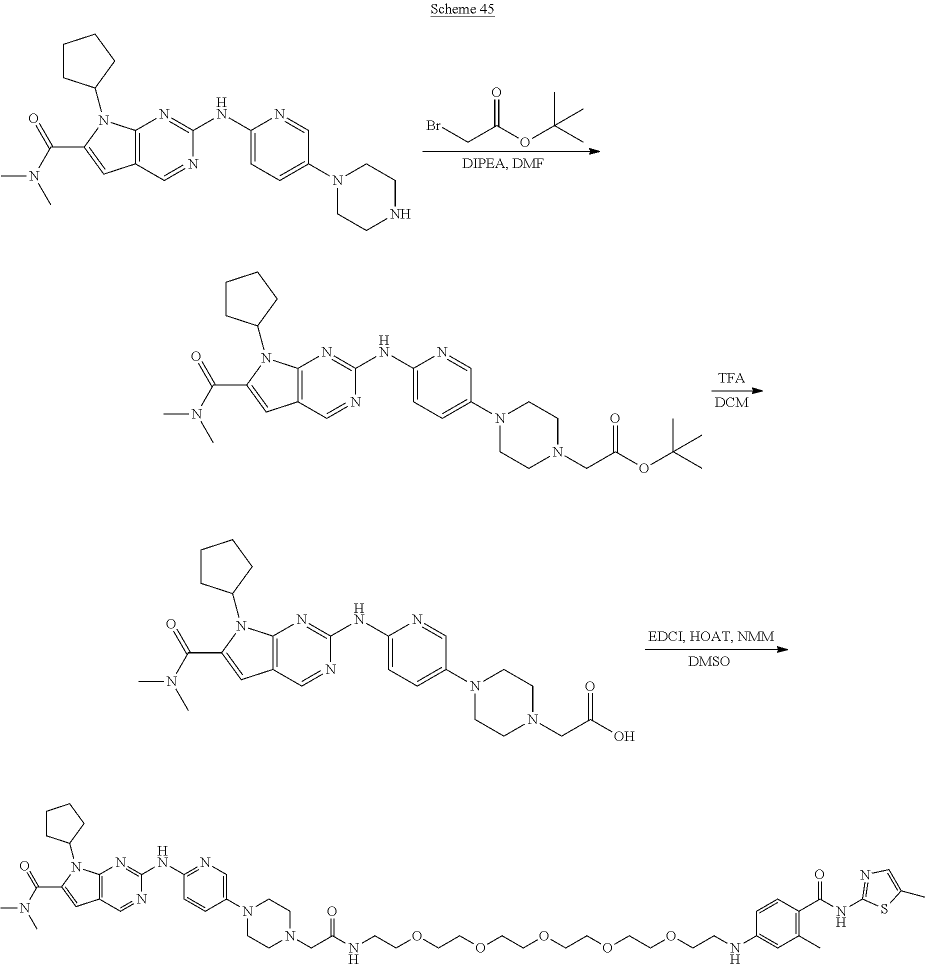

CPD-023 was synthesized following the standard procedures for preparing CPD-020 (2.3 mg, 23% yield over 3 steps). MS (ESI) m/z=985.7 [M+H]⁺.

### Example 046. N⁴-(9-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)nonyl)-2-methyl-N¹-(5-methylthiazol-2-yl)terephthalamide (CPD-024)

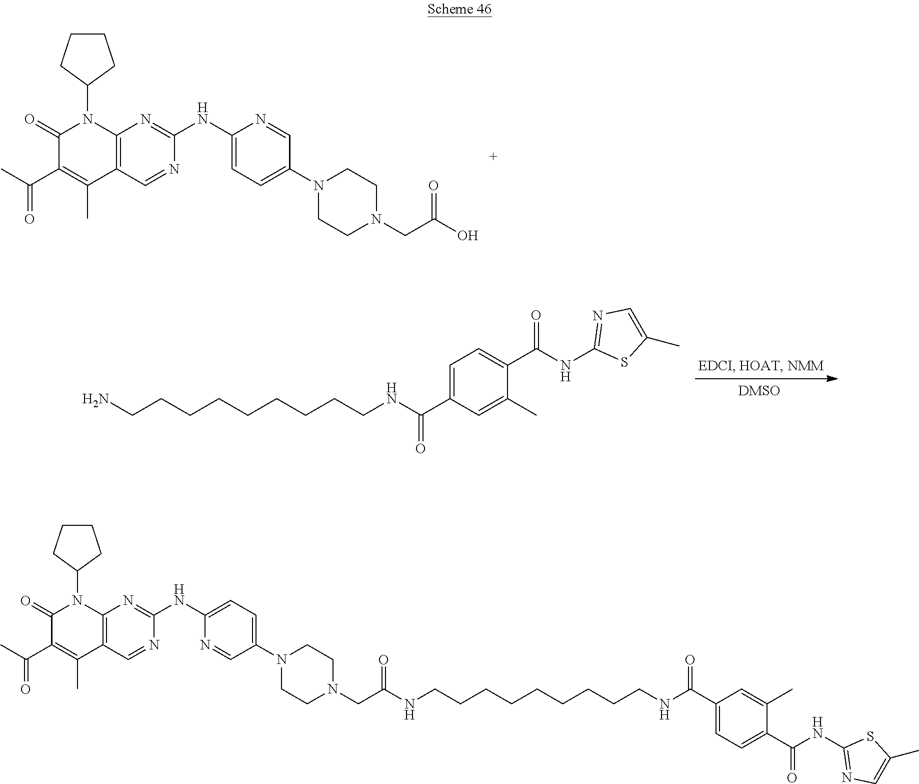

CPD-024 was synthesized following the standard procedure for preparing CPD-008 (1.4 mg, 16% yield). MS (ESI) m/z=904.5 [M+H]⁺.

### Example 047. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-2-methyl-N-(p-tolyl)benzamide (CPD-025)

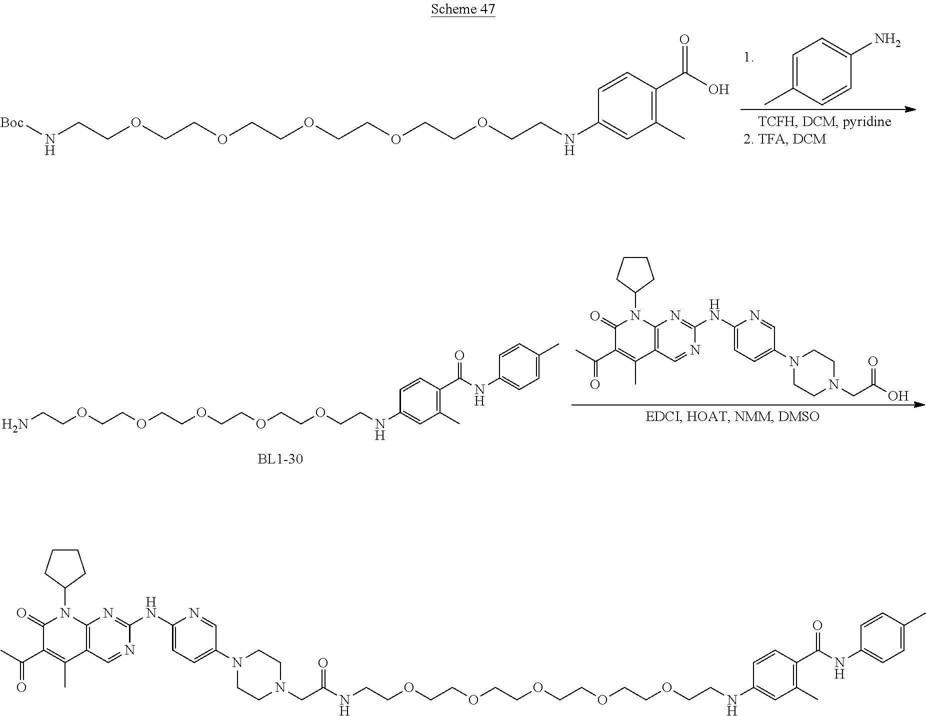

CPD-025 was synthesized following the standard procedures for preparing CPD-001 (1.5 mg, 8% yield over 3 steps). MS (ESI) m/z=991.6 [M+H]⁺.

### Example 048. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-2-methyl-N-(6-methylpyridin-3-yl)benzamide (CPD-026)

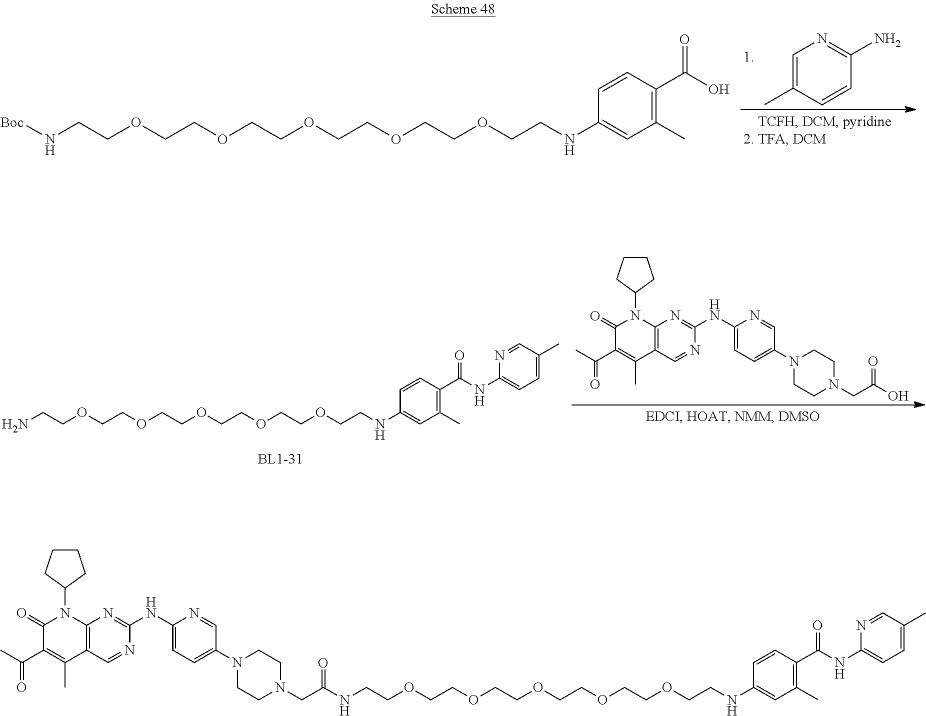

CPD-026 was synthesized following the standard procedures for preparing CPD-001 (1.7 mg, 9% yield over 3 steps). MS (ESI) m/z=992.6 [M+H]⁺.

### Example 049. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-2-methyl-N-phenylbenzamide (CPD-027)

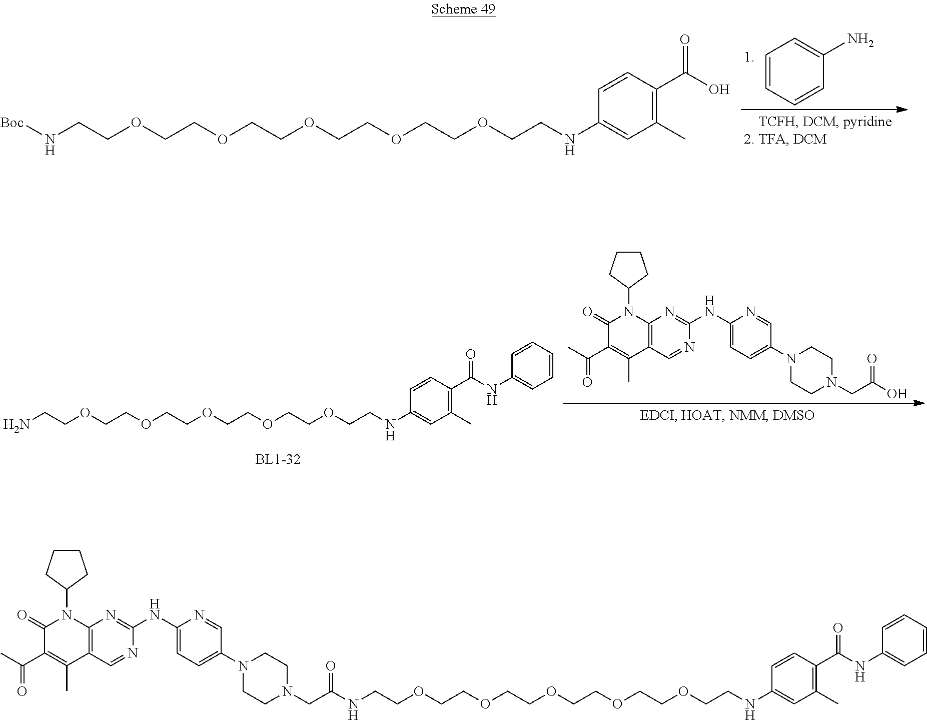

CPD-027 was synthesized following the standard procedures for preparing CPD-001 (1.5 mg, 8% yield over 3 steps). MS (ESI) m/z=977.6 [M+H]⁺.

### Example 050. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-N-(5-fluorothiazol-2-yl)-2-methylbenzamide (CPD-028)

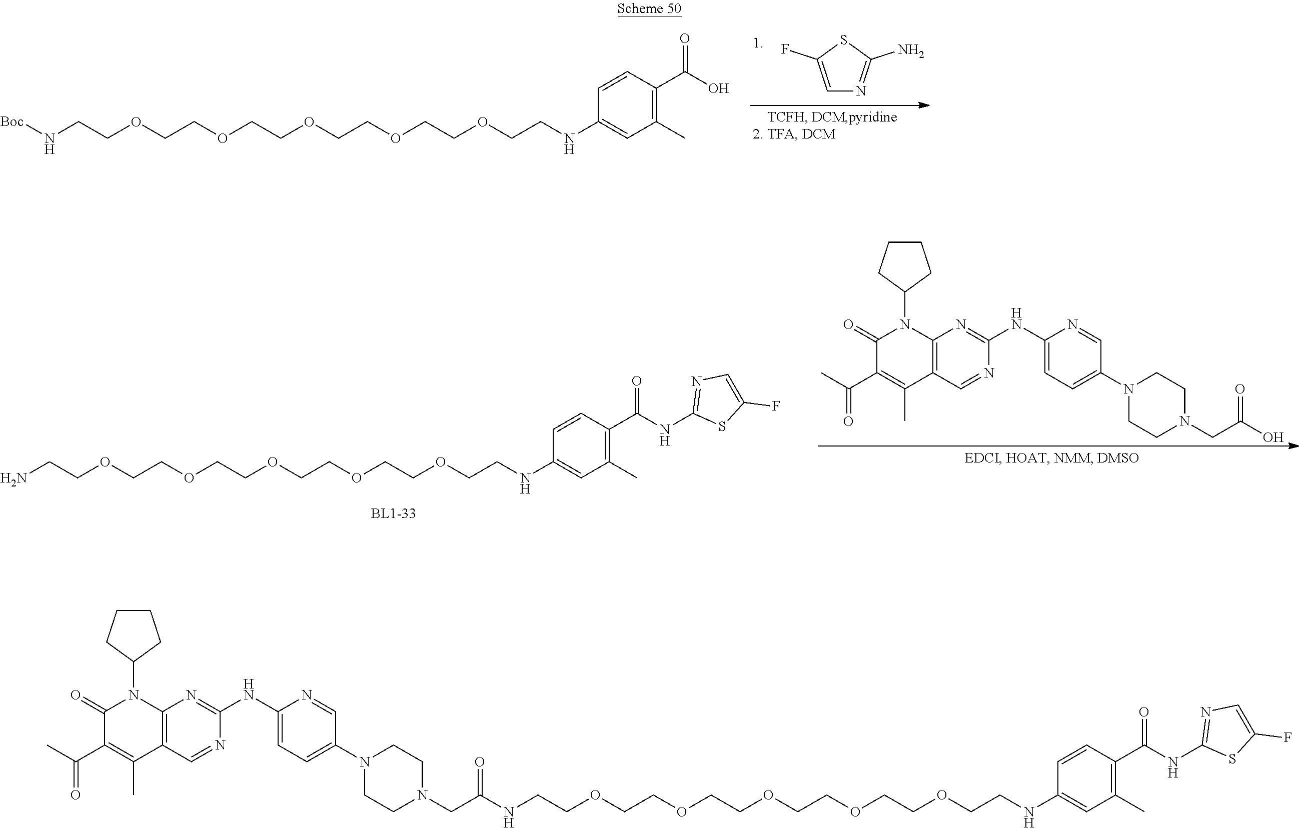 [SMILES: Cc1cc(NCCOCCOCCOCCOCCOCCNC(=O)OC(C)(C)C)ccc1C(=O)O ; Nc1ncc(F)s1 ; Cc1cc(NCCOCCOCCOCCOCCOCCN)ccc1C(=O)Nc1ncc(F)s1 ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)NCCOCCOCCOCCOCCOCCNc5ccc(C(=O)Nc6ncc(F)s6)c(C)c5)CC4)cn3)nc2n(C2CCCC2)c1=O]

CPD-028 was synthesized following the standard procedures for preparing CPD-001 (0.7 mg, 4% yield over 3 steps). MS (ESI) m/z=1002.5 [M+H]⁺.

### Example 051. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-N-(5-cyclopropylthiazol-2-yl)-2-methylbenzamide (CPD-029)

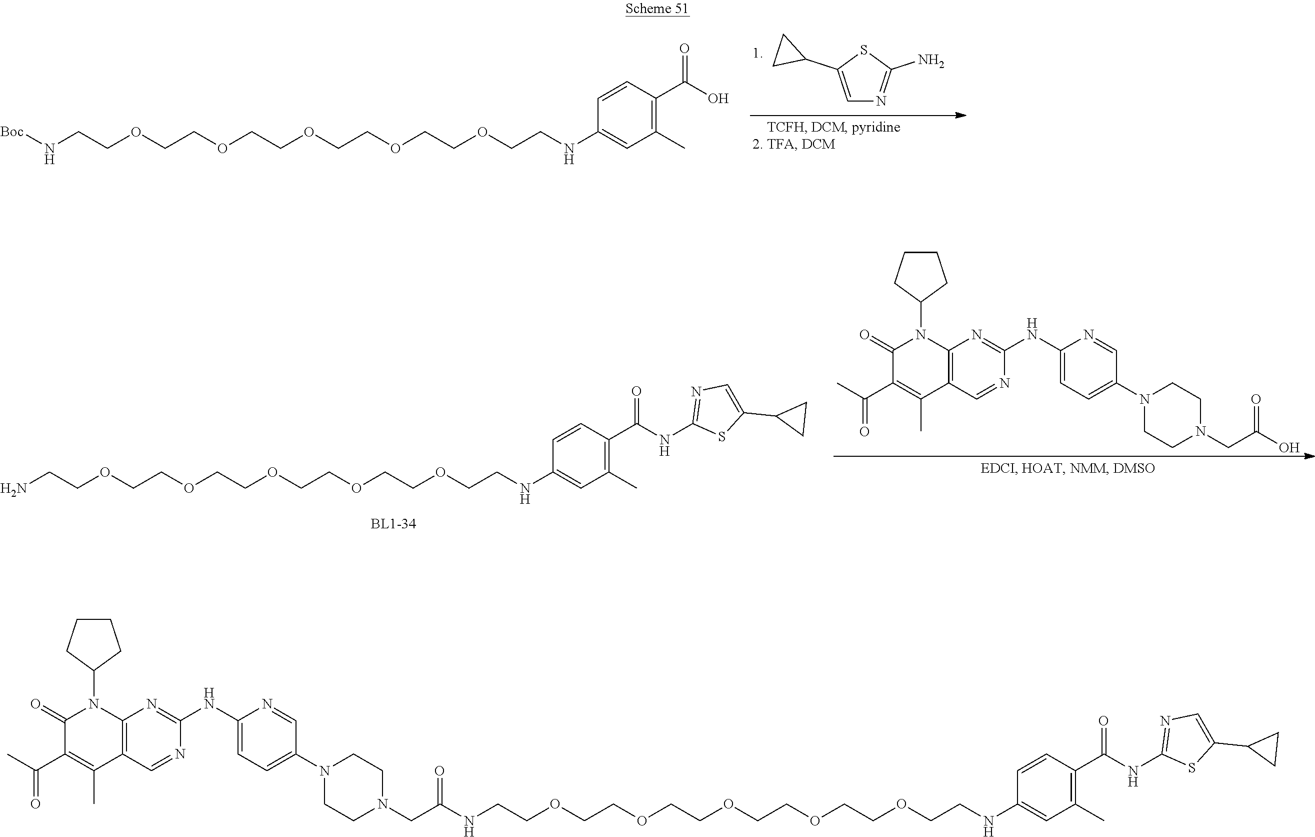

CPD-029 was synthesized following the standard procedures for preparing CPD-001 (2.1 mg, 11% yield over 3 steps). MS (ESI) m/z=1024.6 [M+H]⁺.

### Example 052. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-N-(5-methoxythiazol-2-yl)-2-methylbenzamide (CPD-030)

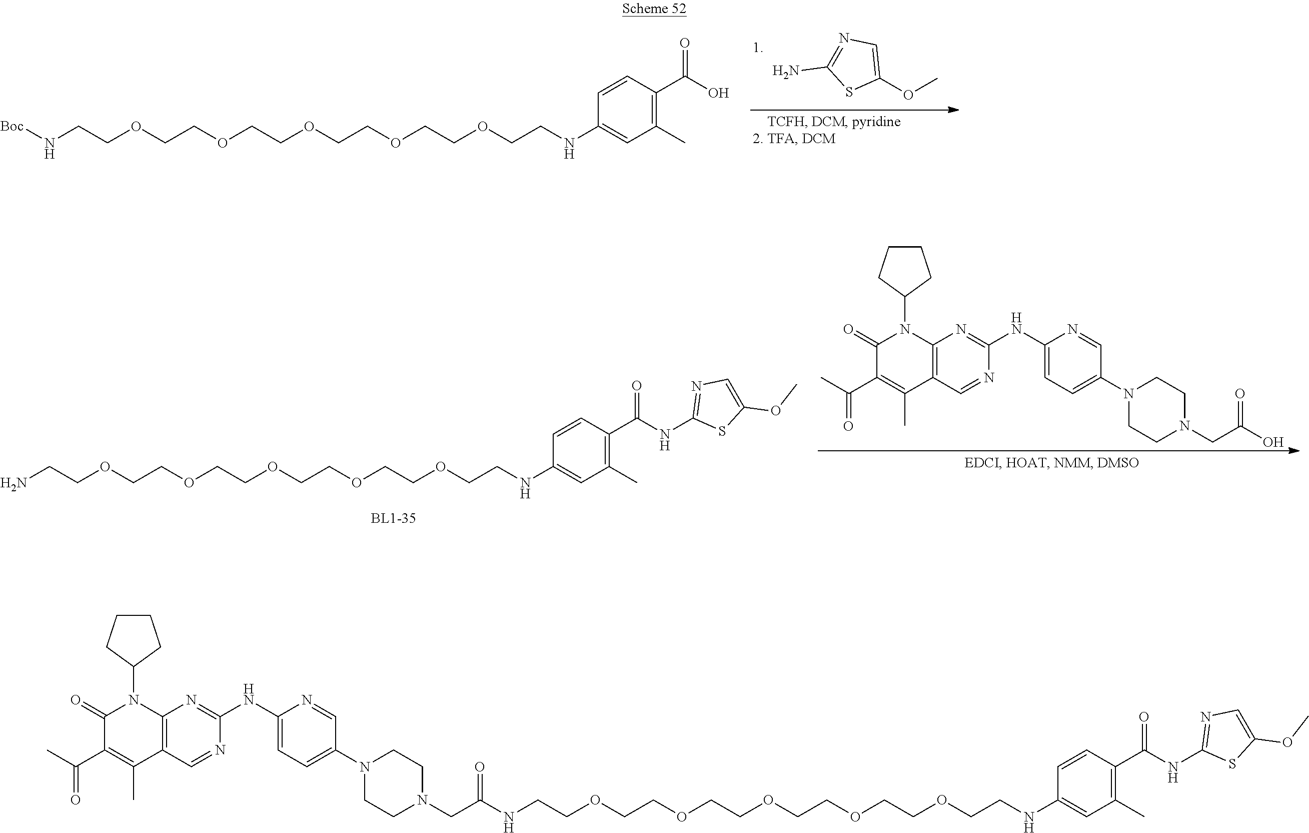

CPD-030 was synthesized following the standard procedures for preparing CPD-001 (5.2 mg, 26% yield over 3 steps). MS (ESI) m/z=1014.5 [M+H]⁺.

### Example 053. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-N-(4,5-dimethylthiazol-2-yl)-2-methylbenzamide (CPD-031)

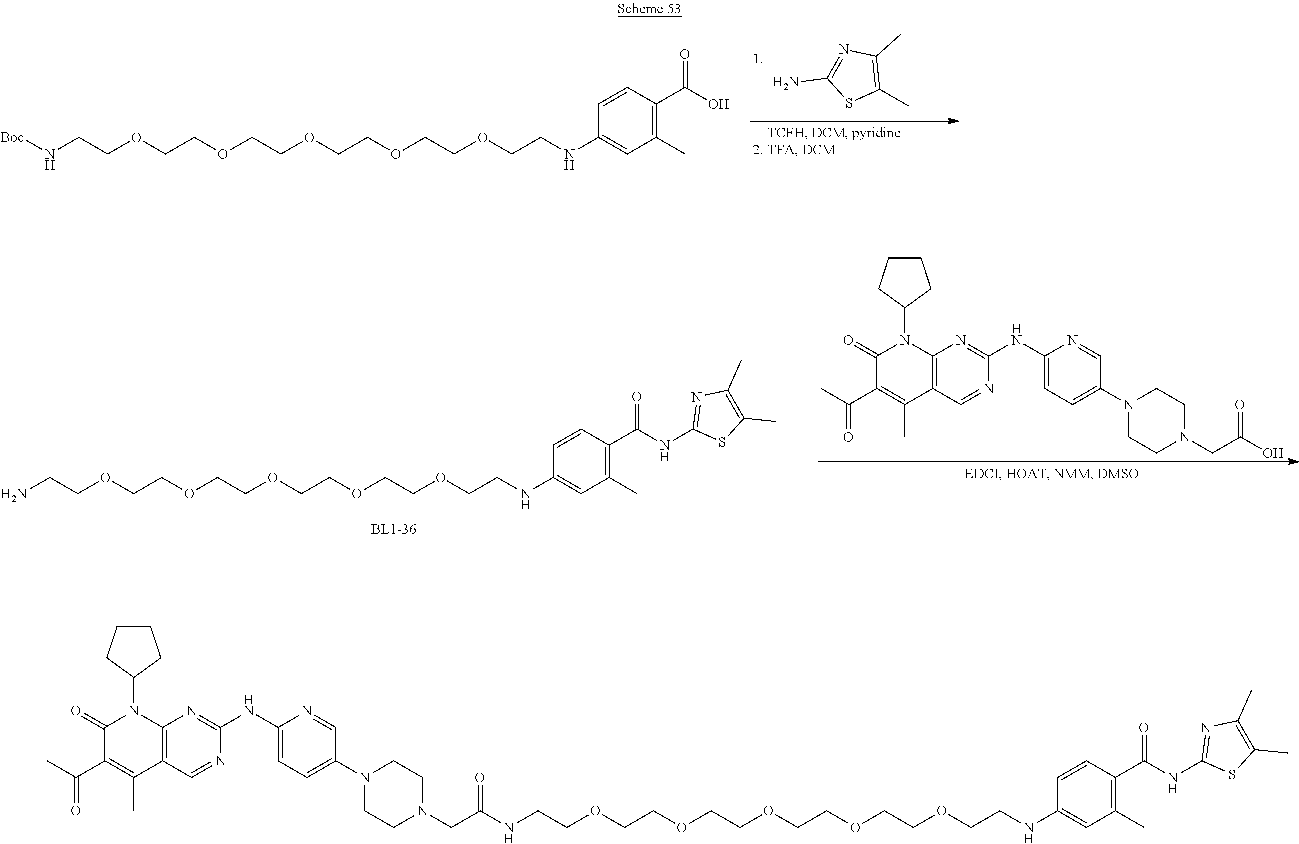

CPD-031 was synthesized following the standard procedures for preparing CPD-001 (1.8 mg, 9% yield over 3 steps). MS (ESI) m/z=1012.6 [M+H]⁺.

### Example 054. Methyl 2-(4-((1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-2-methylbenzamido)thiazole-5-carboxylate (CPD-032)

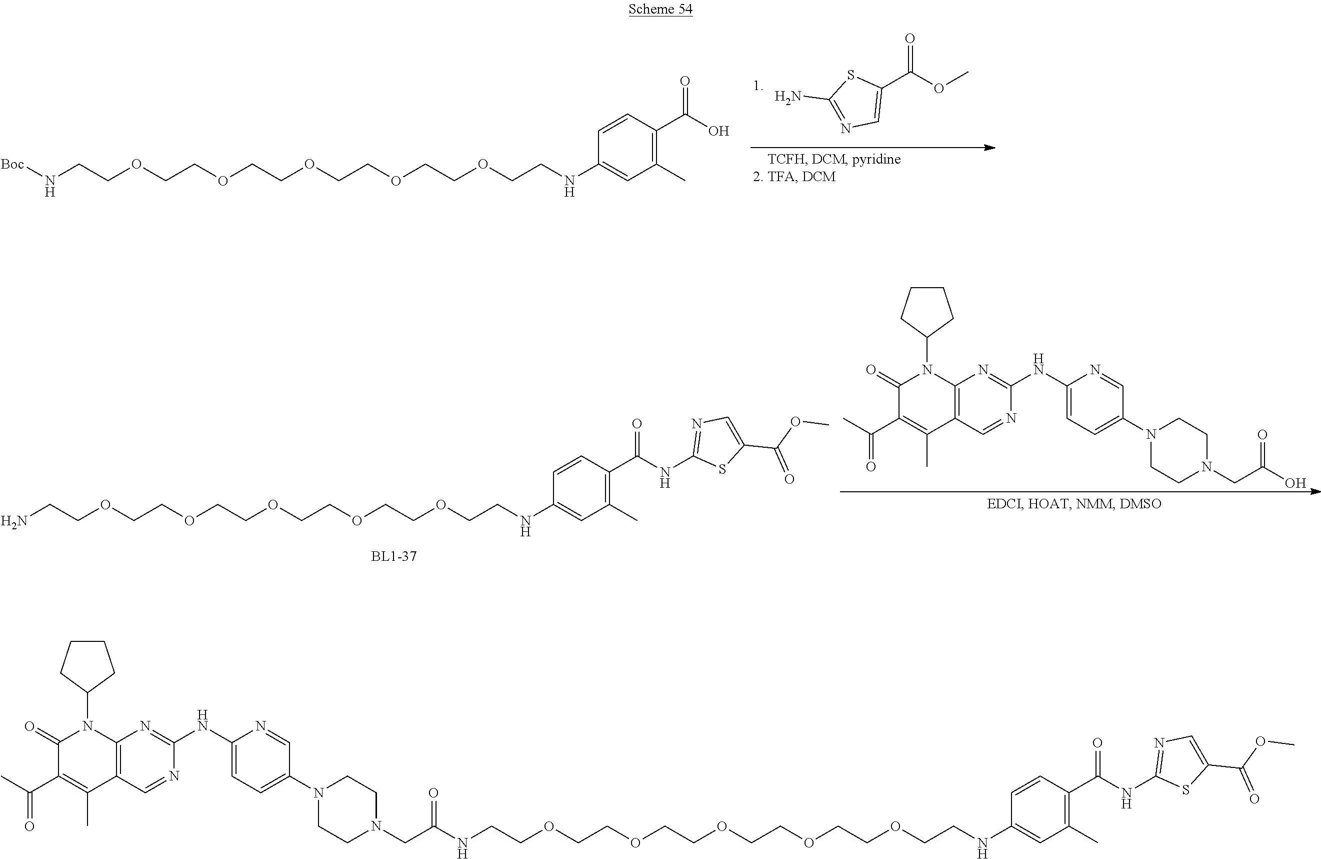

CPD-032 was synthesized following the standard procedures for preparing CPD-001 (3.1 mg, 15% yield over 3 steps). MS (ESI) m/z=1042.5 [M+H]⁺.

### Example 055. Methyl 2-(4-((1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-2-methylbenzamido)-5-methylthiazole-4-carboxylate (CPD-033)

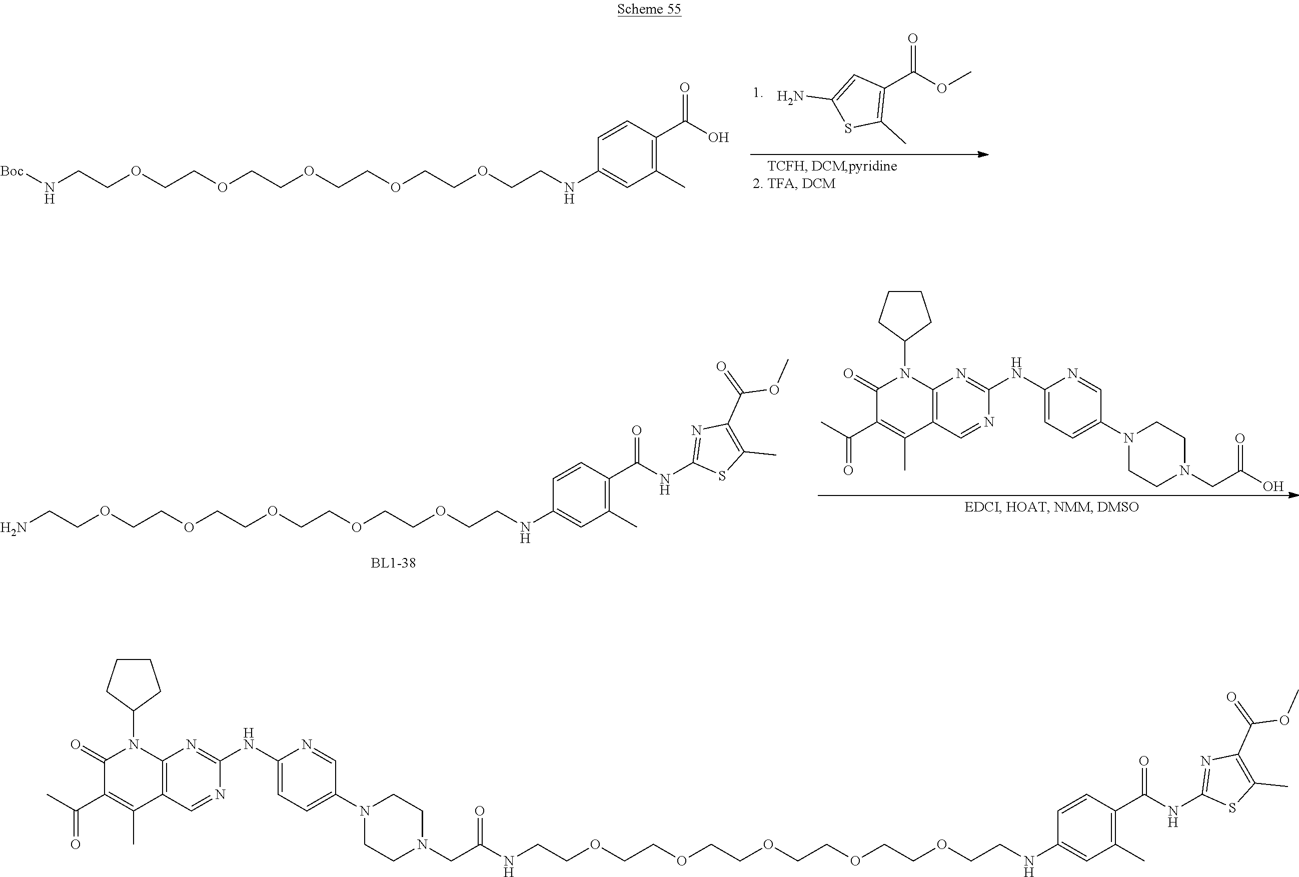

CPD-033 was synthesized following the standard procedures for preparing CPD-001 (3.8 mg, 19% yield over 3 steps). MS (ESI) m/z=1056.5 [M+H]⁺.

### Example 056. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl-2-oxo-6,9,12,15,18-pentaoxa-3-azaicosan-20-yl)amino)-2-methyl-N-(5-methyl-4-phenylthiazol-2-yl)benzamide (CPD-034)

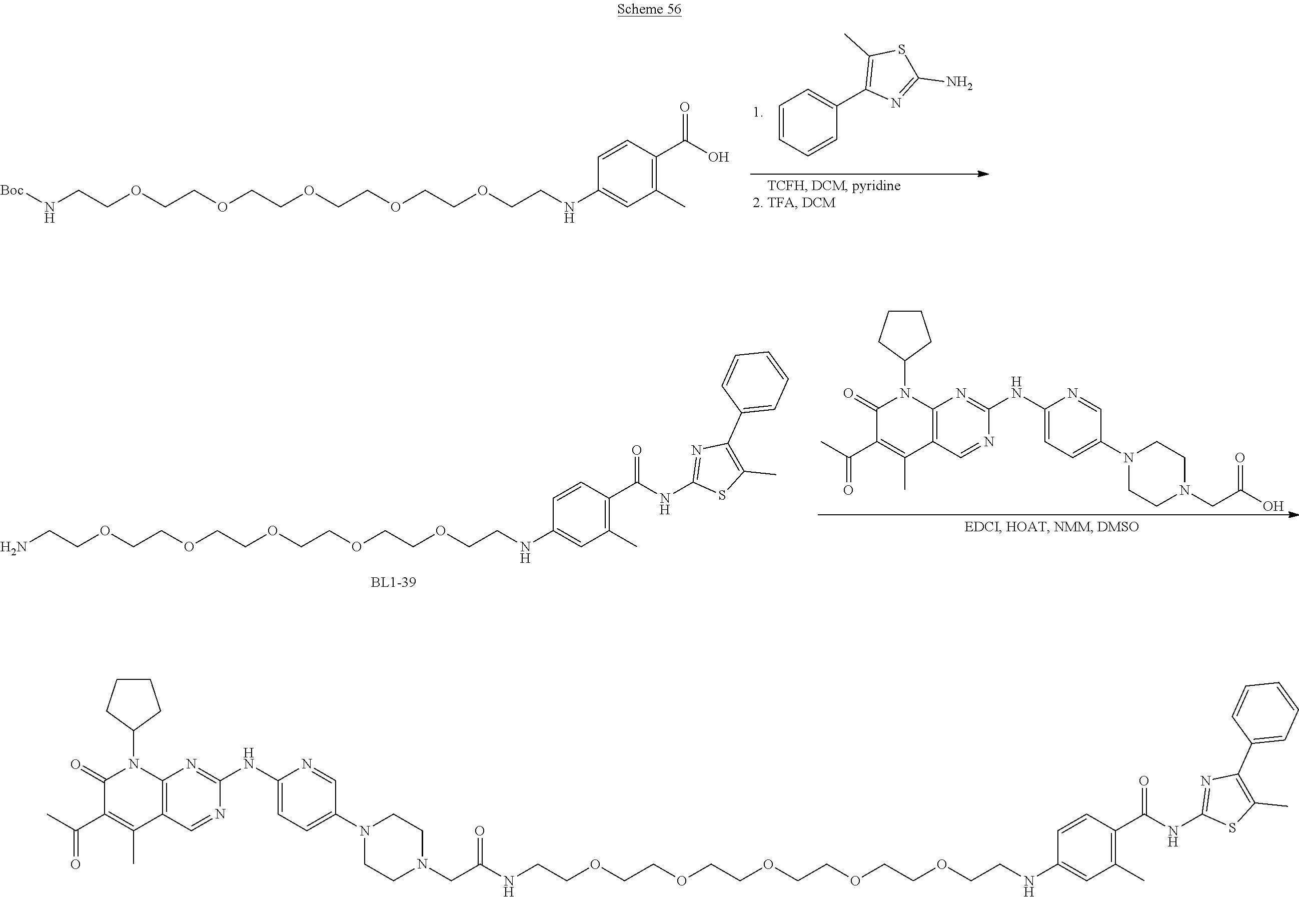 [SMILES: Cc1cc(NCCOCCOCCOCCOCCOCCNC(=O)OC(C)(C)C)ccc1C(=O)O ; Cc1sc(N)nc1-c1ccccc1 ; Cc1cc(NCCOCCOCCOCCOCCOCCN)ccc1C(=O)Nc1nc(-c2ccccc2)c(C)s1 ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)NCCOCCOCCOCCOCCOCCNc5ccc(C(=O)Nc6nc(-c7ccccc7)c(C)s6)c(C)c5)CC4)cn3)nc2n(C2CCCC2)c1=O]

CPD-034 was synthesized following the standard procedures for preparing CPD-001 (4.8 mg, 23% yield over 3 steps). MS (ESI) m/z=1074.6 [M+H]⁺.

### Example 057. 4-((2-((9-(2-(4-(6-((6-Acetyl-8-cyclopentyl-5-methyl-7-oxo-7,8-dihydropyrido[2,3-d]pyrimidin-2-yl)aminopyridin-3-yl)piperazin-1-yl)acetamido)nonyl)amino)-2-oxoethyl)amino)-2-methyl-N-(5-methylthiazol-2-yl)benzamide (CPD-035)

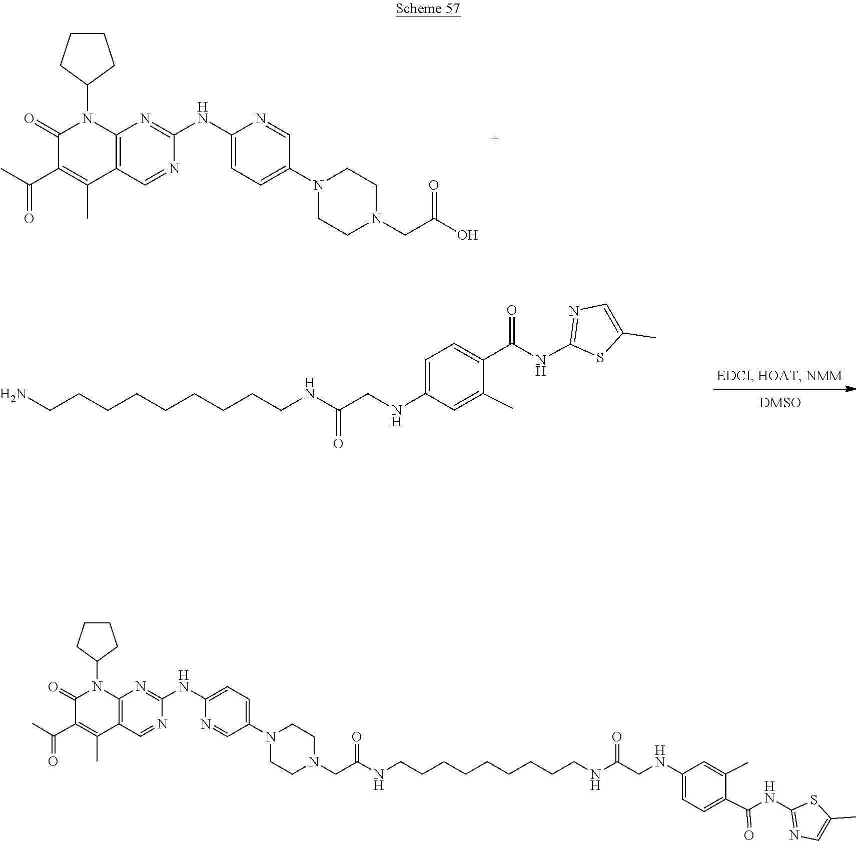

CPD-035 was synthesized following the standard procedure for preparing CPD-008 (4.7 mg, 51% yield). MS (ESI) m/z=933.5 [M+H]⁺.

### Example 058. 4-(2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-2-oxoethoxy)-2-methyl-N-(5-methylthiazol-2-yl)benzamide (CPD-036)

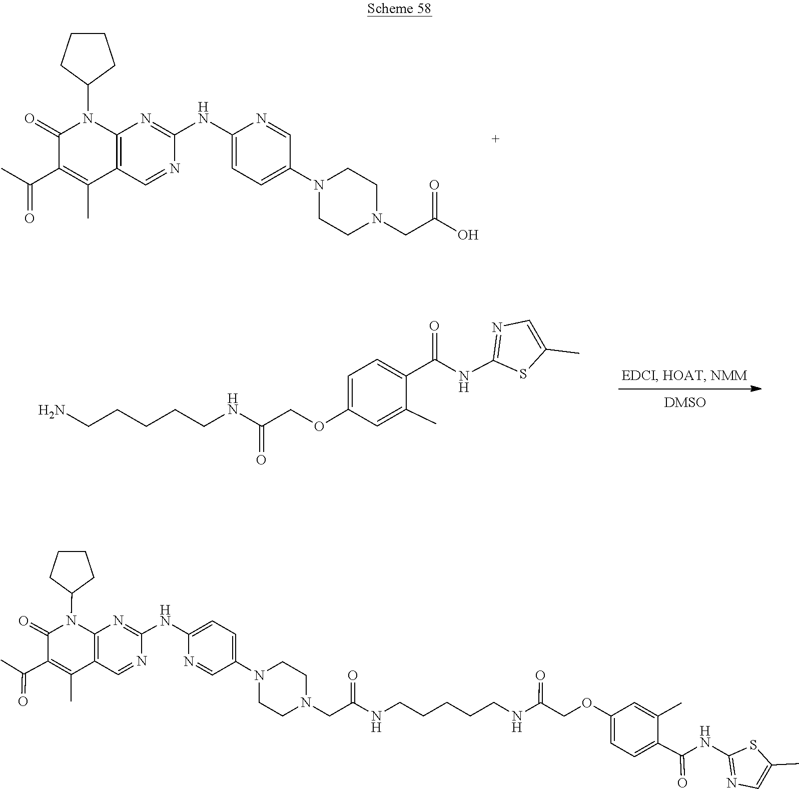

CPD-036 was synthesized following the standard procedure for preparing CPD-008 (3.6 mg, 42% yield). MS (ESI) m/z=878.5 [M+H]⁺.

### Example 059. 4-(2-((7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-2-oxoethoxy)-2-methyl-N-(5-methylthiazol-2-yl)benzamide (CPD-037)

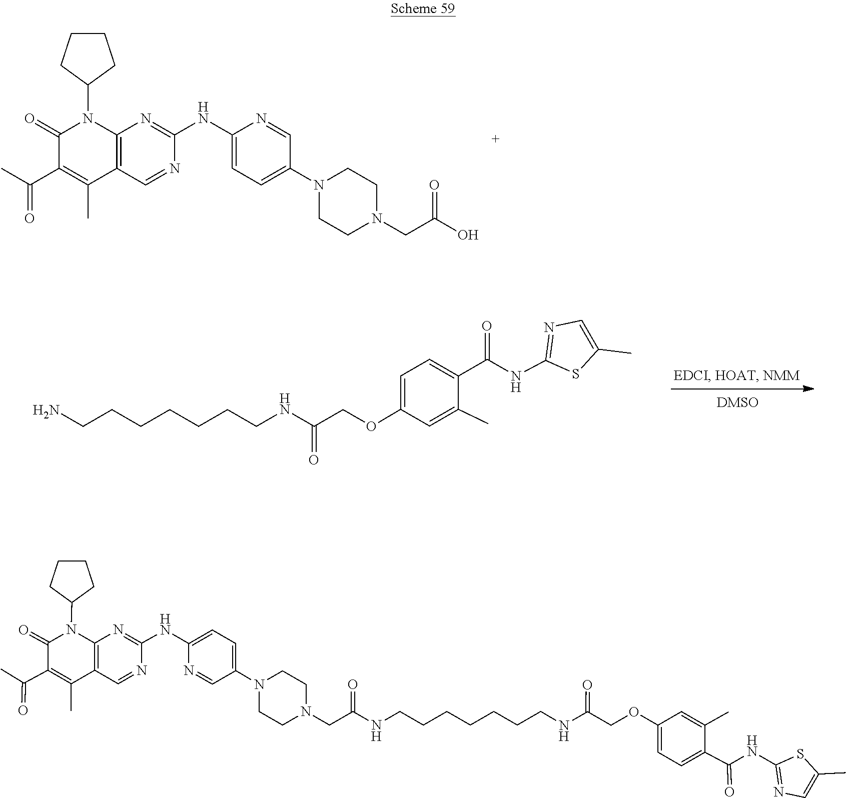

CPD-037 was synthesized following the standard procedure for preparing CPD-008 (3.9 mg, 44% yield). MS (ESI) m/z=906.5 [M+H]⁺.

### Example 060. 4-(2-((9-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)nonyl)amino)-2-oxoethoxy)-2-methyl-N-(5-methylthiazol-2-yl)benzamide (CPD-038)

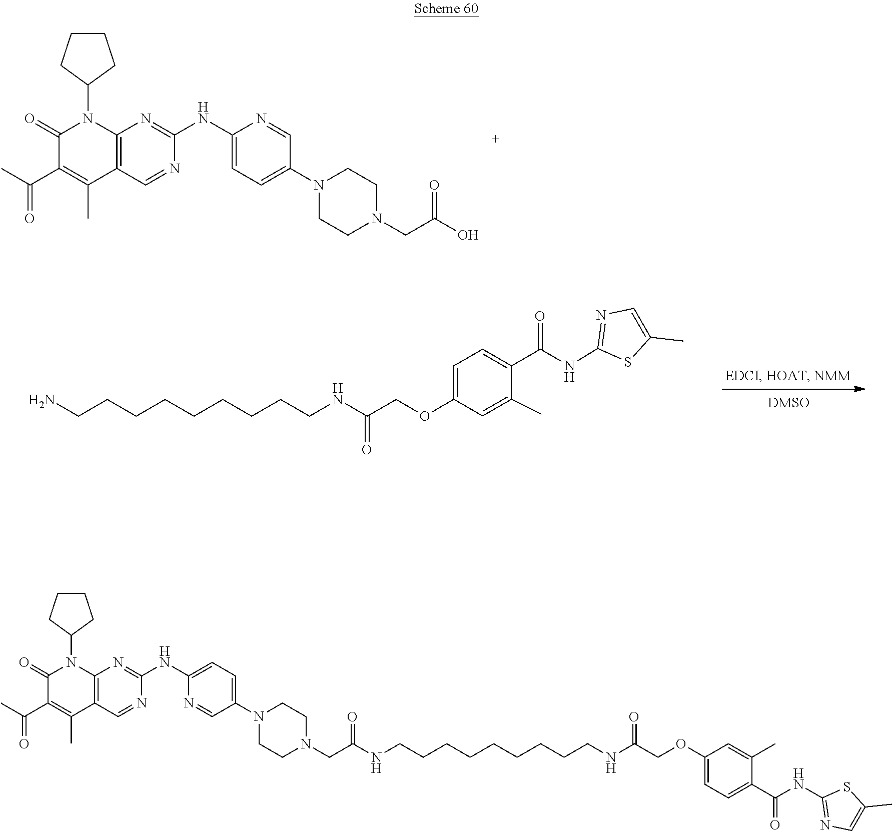

CPD-038 was synthesized following the standard procedure for preparing CPD-008 (3.9 mg, 42% yield). MS (ESI) m/z=934.5 [M+H]⁺.

### Example 061. 4-((14-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2,13-dioxo-6,9-dioxa-3,12-diazatetradecyl)oxy)-2-methyl-N-(5-methylthiazol-2-yl)benzamide (CPD-039)

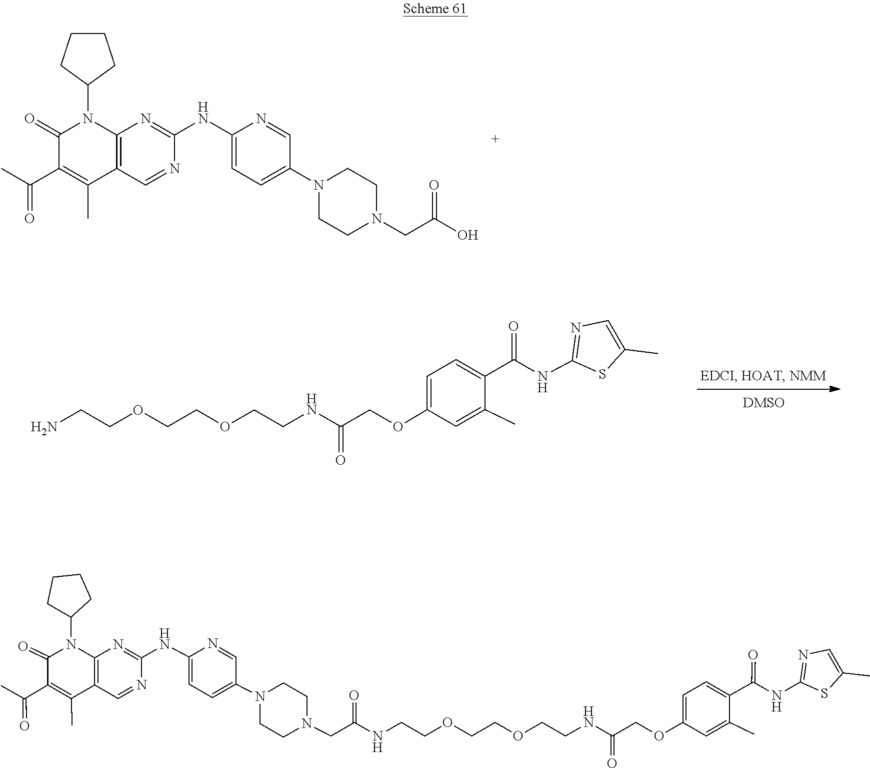

CPD-039 was synthesized following the standard procedure for preparing CPD-008 (5.6 mg, 42% yield). MS (ESI) m/z=924.5 [M+H]⁺.

### Example 062. 4-((17-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2,16-dioxo-6,9,12-trioxa-3,15-diazaheptadecyl)oxy)-2-methyl-N-(5-methylthiazol-2-yl)benzamide (CPD-040)

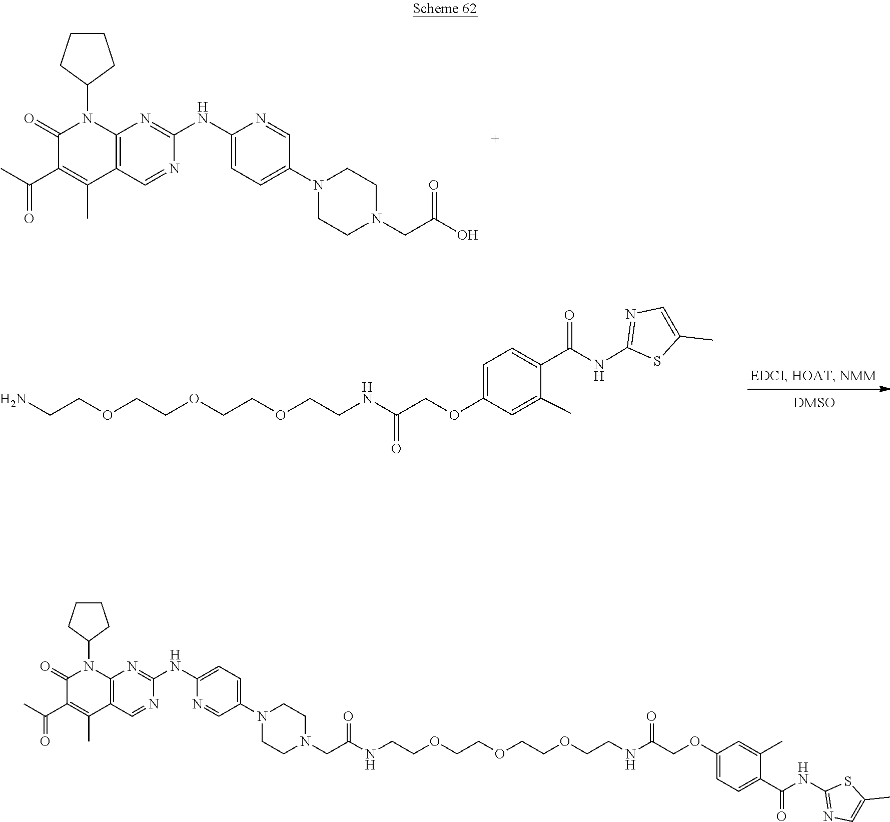

CPD-040 was synthesized following the standard procedure for preparing CPD-008 (4.7 mg, 49% yield). MS (ESI) m/z=968.5 [M+H]⁺.

### Example 063. 4-((20-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2,19-dioxo-6,9,12,15-tetraoxa-3,18-diazaicosyl)oxy)-2-methyl-N-(5-methylthiazol-2-yl)benzamide (CPD-041)

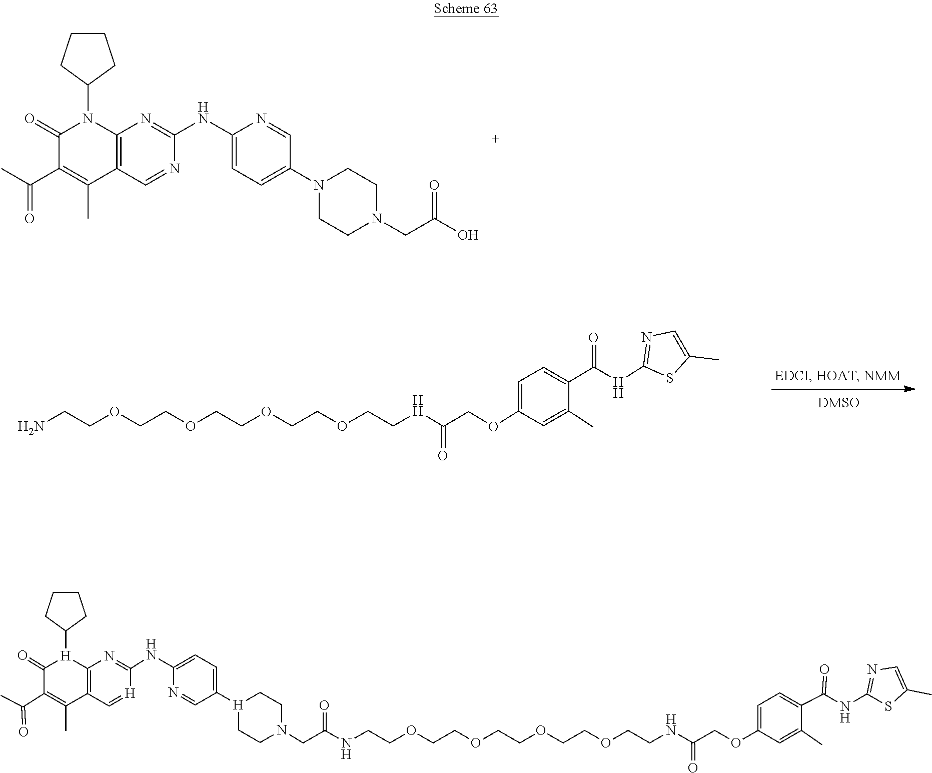

CPD-041 was synthesized following the standard procedure for preparing CPD-008 (4.9 mg, 49% yield). MS (ESI) m/z=1012.5 [M+H]⁺.

### Example 064. 4-((1-(4-(6-((6-Acetyl-8-cyclopentyl-5-methyl-7-oxo-7,8-dihydropyrido[2,3-d]pyrimidin-2-yl)(methyl)amino)pyridin-3-yl)piperazin-1-yl)-2-oxo-6,9,12,15,18-pentaoxa-3-azaicosan-20-yl)amino)-2-methyl-N-(5-methylthiazol-2-yl)benzamide (CPD-042)

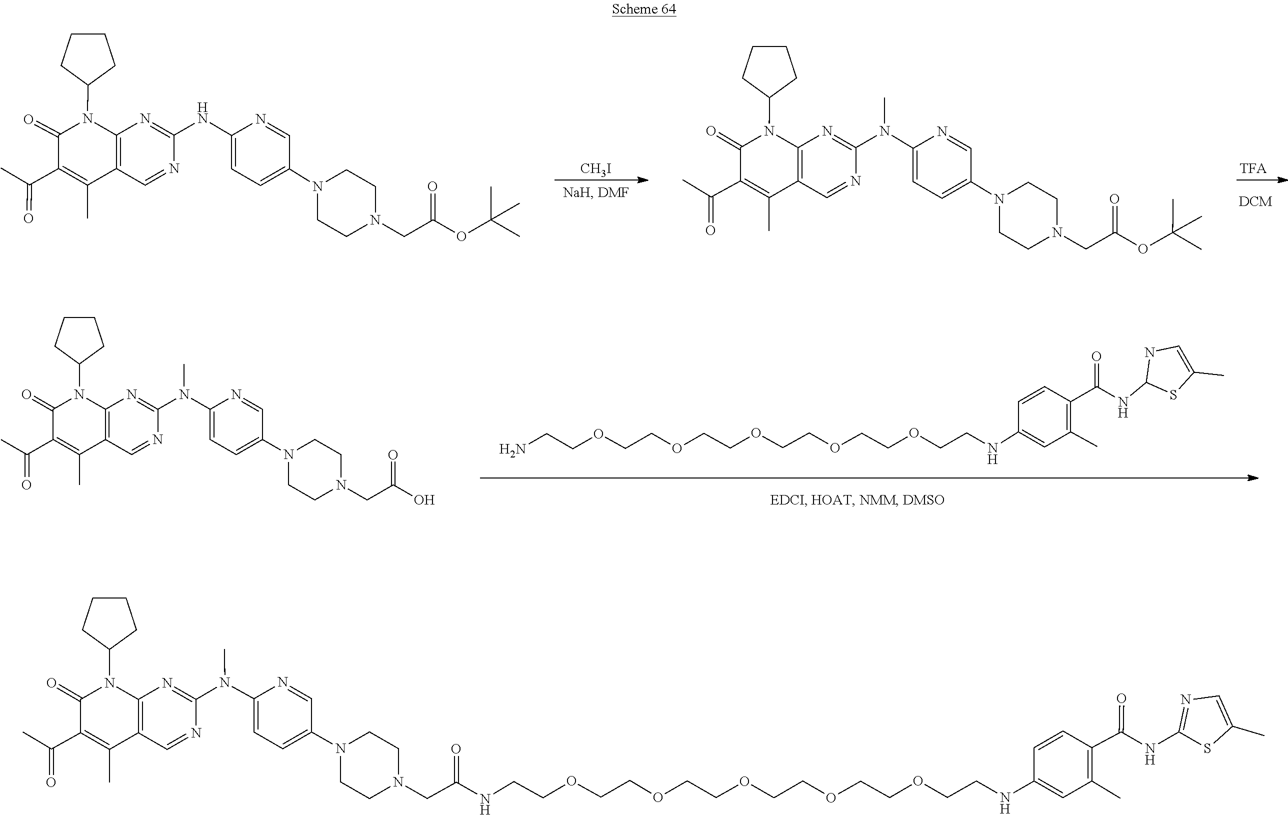

### Step 1. Synthesis of tert-butyl 2-(4-(6-((6-acetyl-8-cyclopentyl-5-methyl-7-oxo-7,8-dihydropyrido[2,3-d]pyrimidin-2-yl)(methyl)amino)pyridin-3-yl)piperazin-1-yl)acetate

To a solution of tert-butyl 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetate (66 mg, 0.12 mmol) in THF (5 mL) was added NaH (7.2 mg, 0.18 mmol) at 0° C. under nitrogen atmosphere. After the reaction was stirred at 0° C. for 0.5 h, CH₃I (34 mg, 0.24 mmol) in THF (2 mL) was added dropwise at 0° C. The reaction mixture was stirred at rt for 16 h, before it was poured into water (20 mL) and extracted with ethyl acetate (3×10 mL). The combined organic layers were washed with saturated brine (20 mL), dried over anhydrous sodium sulfate, filtered and evaporated under reduced pressure. The resulting residue was purified by prep-TLC to provide the desired product (6.7 mg, 10% yield) as a yellow solid. MS (ESI) m/z=576.4 [M+H]⁺.

### Step 2. Synthesis of 2-(4-(6-((6-acetyl-8-cyclopentyl-5-methyl-7-oxo-7,8-dihydropyrido[2,3-d]pyrimidin-2-yl)(methyl)amino)pyridin-3-yl)piperazin-1-yl)acetic acid

To a solution of tert-butyl 2-(4-(6-((6-acetyl-8-cyclopentyl-5-methyl-7-oxo-7,8-dihydropyrido[2,3-d]pyrimidin-2-yl)(methyl)amino)pyridin-3-yl)piperazin-1-yl)acetate (7.6 mg, 0.01 mmol) in DCM (2 mL) was added TFA (1 mL) at 0° C. After the reaction mixture was stirred at rt for 1 h, it was concentrated to provide the crude product (6.5 mg, 100% yield) as a yellow solid. This compound was used directly in the next step without further purification. MS (ESI) m/z=520.3 [M+H]⁺.

### Step 3. Synthesis of 4-((1-(4-(6-((6-acetyl-8-cyclopentyl-5-methyl-7-oxo-7,8-dihydropyrido[2,3-d]pyrimidin-2-yl)(methyl)amino)pyridin-3-yl)piperazin-1-yl)-2-oxo-6,9,12,15,18-pentaoxa-3-azaicosan-20-yl)amino)-2-methyl-N-(5-methylthiazol-2-yl)benzamide

A solution of 2-(4-(6-((6-acetyl-8-cyclopentyl-5-methyl-7-oxo-7,8-dihydropyrido[2,3-d]pyrimidin-2-yl)(methyl)amino)pyridin-3-yl)piperazin-1-yl)acetic acid (6.5 mg, 0.01 mmol), 4-((17-amino-3,6,9,12,15-pentaoxaheptadecyl)amino)-2-methyl-N-(5-methylthiazol-2-yl)benzamide (7.6 mg, 0.02 mmol), EDCI (3.8 mg, 0.02 mmol), HOAt (2.7 mg, 0.02 mmol) and NMM (5.1 mg, 0.05 mmol) in DMSO (2 mL) was stirred at rt for 16 h. The reaction mixture was poured into water (10 mL) and extracted with ethyl acetate (3×10 mL). The combined organic layers were washed with saturated brine (20 mL), dried over anhydrous sodium sulfate, filtered and evaporated under reduced pressure. The resulting residue was purified by reverse-phase chromatography and prep-TLC to provide the desired product (5.4 mg, 53% yield) as a yellow solid. MS (ESI) m/z=1012.6 [M+H]⁺.

### Example 065. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-N-(4-isopropyl-5-methylthiazol-2-yl)-2-methylbenzamide (CPD-043)

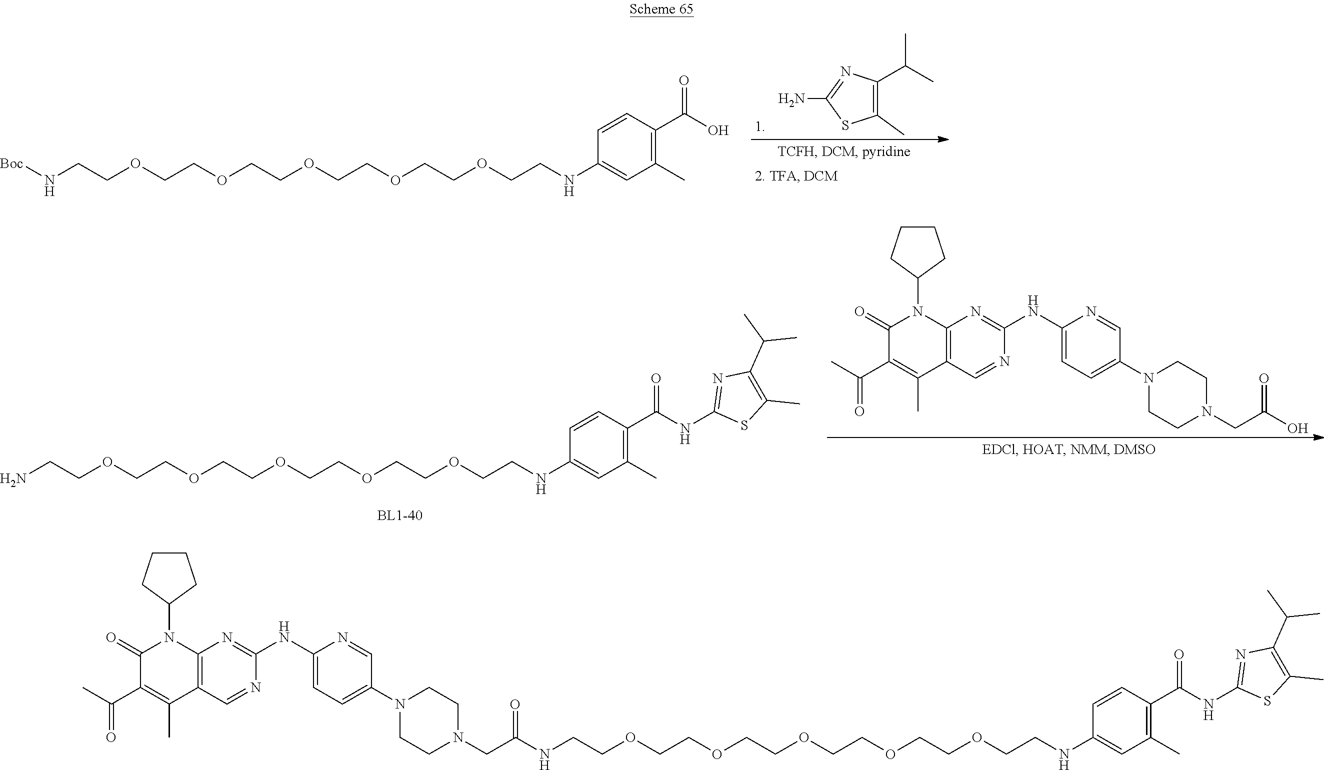

CPD-043 was synthesized following the standard procedures for preparing CPD-001 (2.5 mg, 13% yield over 3 steps). MS (ESI) m/z=1040.6 [M+H]⁺.

### Example 066. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-N-(4-bromo-5-methylthiazol-2-yl)-2-methylbenzamide (CPD-044)

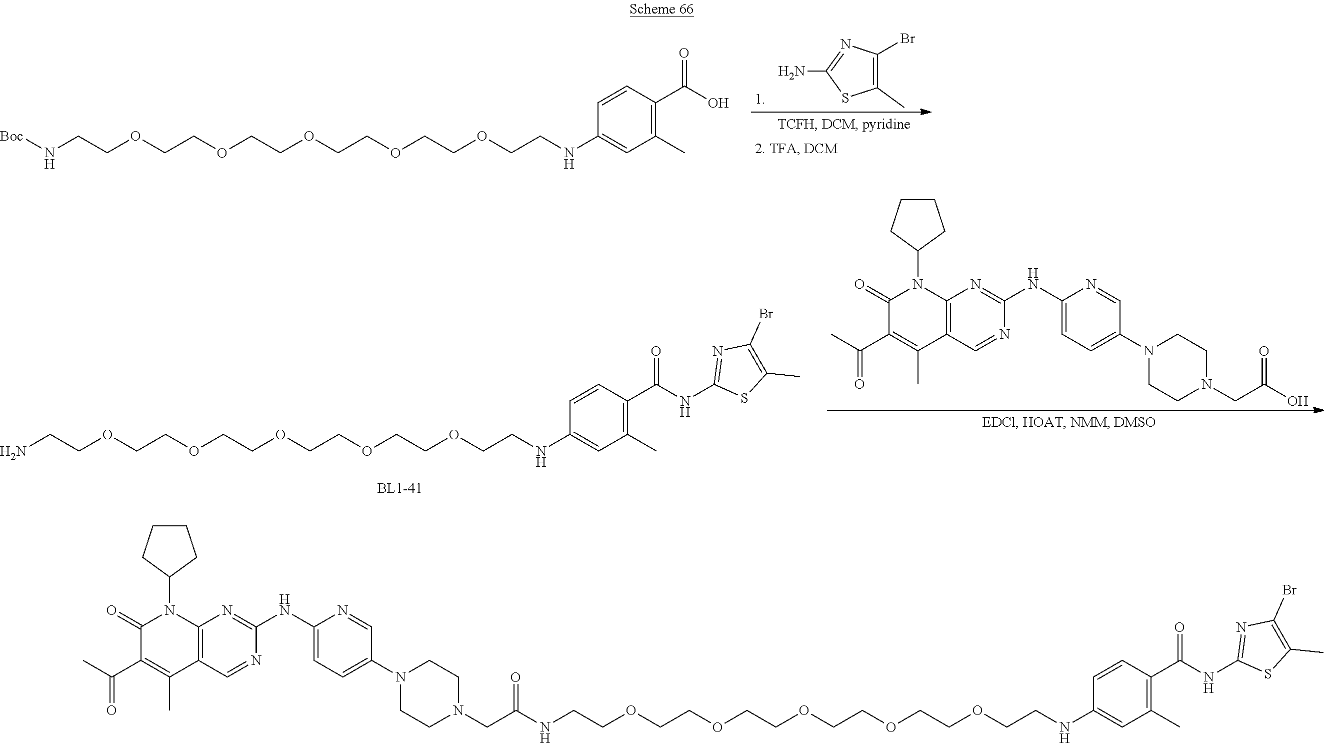

CPD-044 was synthesized following the standard procedures for preparing CPD-001 (3.7 mg, 18% yield over 3 steps). MS (ESI) m/z=1076.4 [M+H]⁺.

### Example 067. N-(4-Acetyl-5-methylthiazol-2-yl)-4-((1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-2-methylbenzamide (CPD-045)

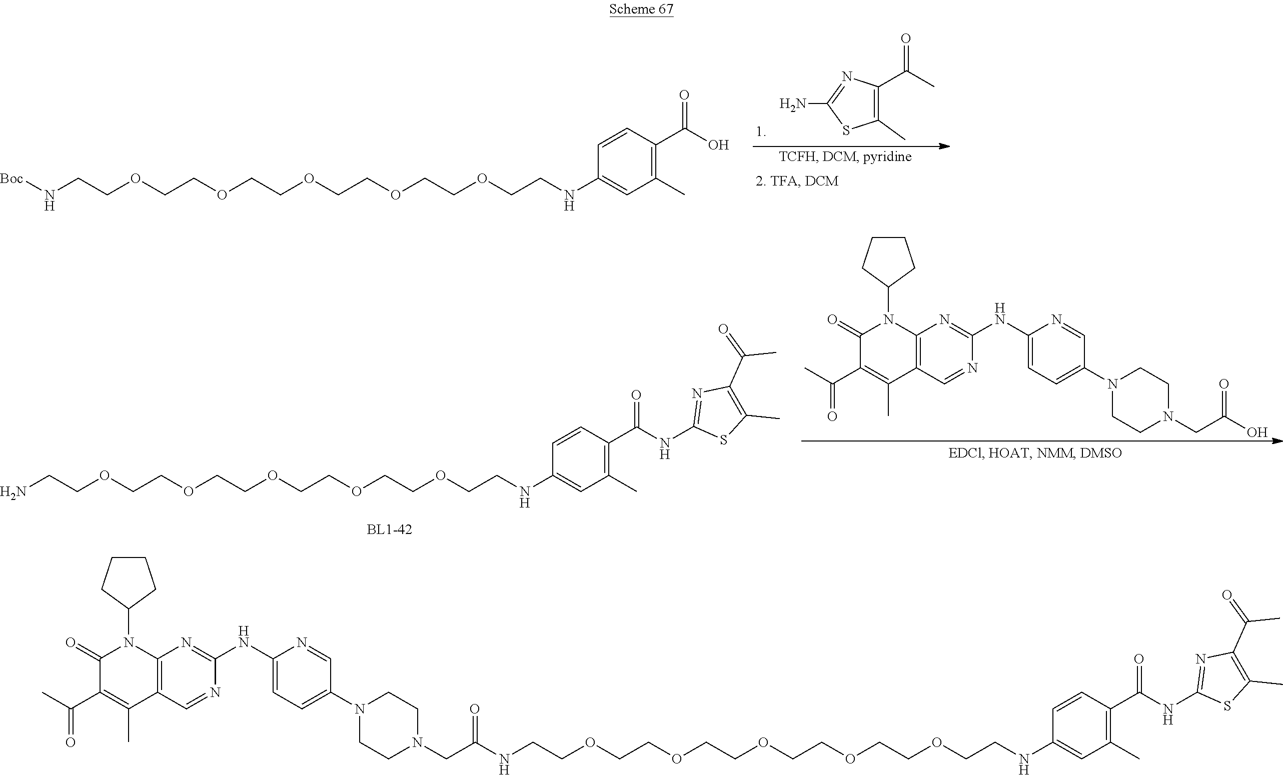

CPD-045 was synthesized following the standard procedures for preparing CPD-001 (2.0 mg, 10% yield over 3 steps). MS (ESI) m/z=1040.6 [M+H]⁺.

### Example 068. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-N-(4-cyclopropyl-5-methylthiazol-2-yl)-2-methylbenzamide (CPD-046)

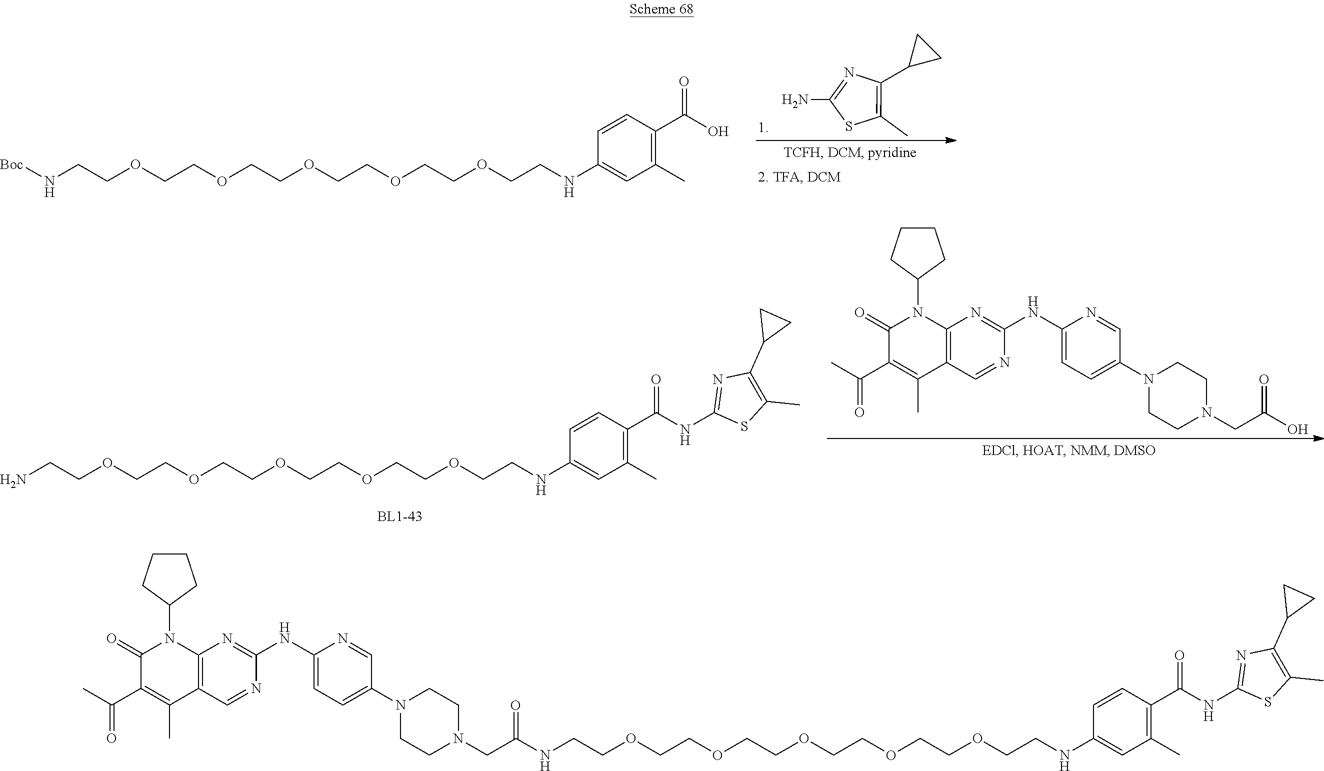

CPD-046 was synthesized following the standard procedures for preparing CPD-001 (1.4 mg, 7% yield over 3 steps). MS (ESI) m/z=1038.6 [M+H]⁺.

### Example 069. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-N-(4-ethyl-5-methylthiazol-2-yl)-2-methylbenzamide (CPD-047)

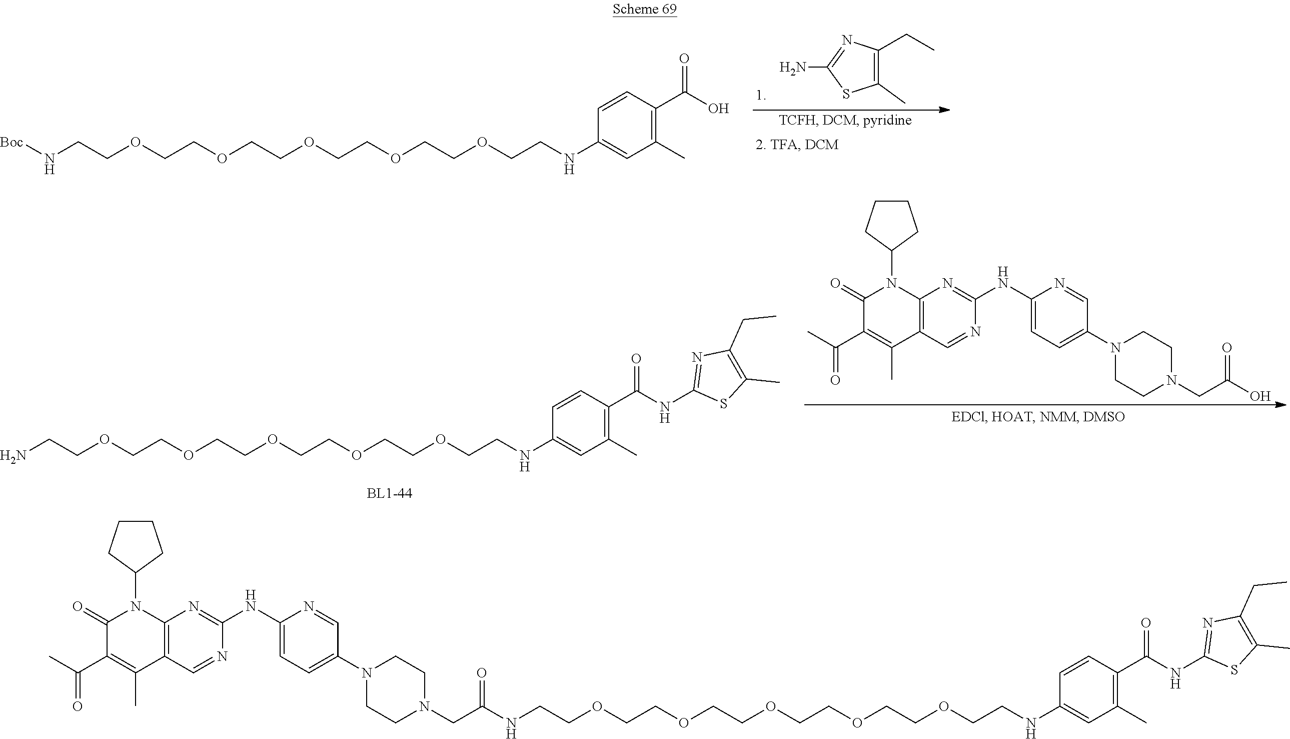

CPD-047 was synthesized following the standard procedures for preparing CPD-001 (2.2 mg, 11% yield over 3 steps). MS (ESI) m/z=1026.6 [M+H]⁺.

### Example 070. N⁴-(7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)-2-methyl-N¹-(5-methylthiazol-2-yl)terephthalamide (CPD-048)

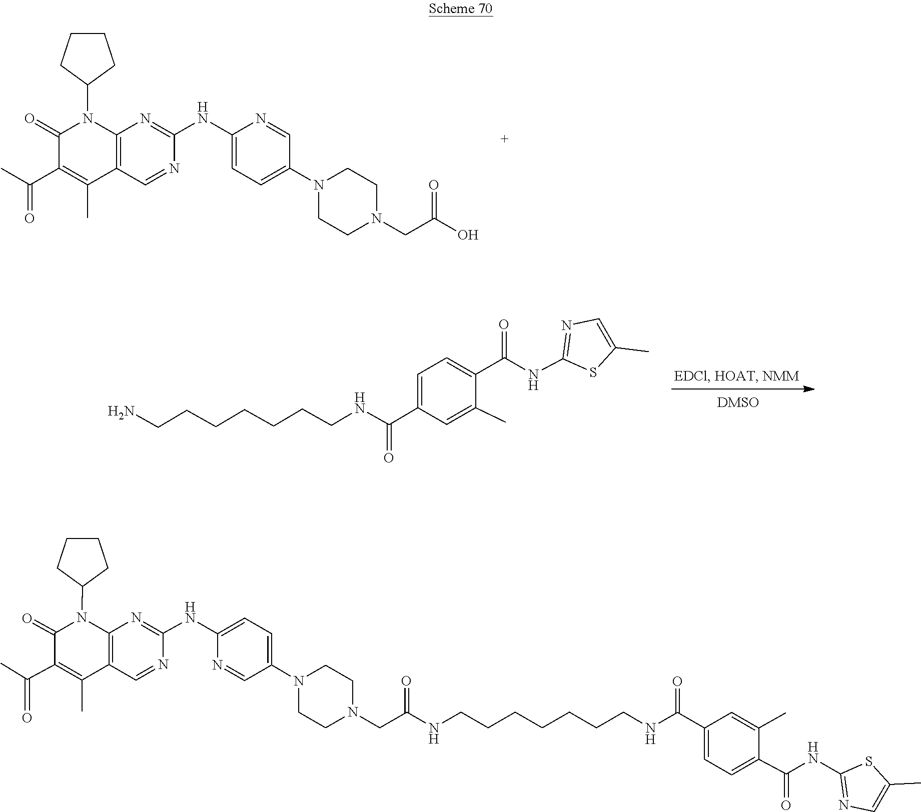 [SMILES: CC(=O)c1c(C)c2cnc(Nc3ccc(N4CCN(CC(=O)O)CC4)cn3)nc2n(C2CCCC2)c1=O ; "+" ; Cc1cnc(NC(=O)c2ccc(C(=O)NCCCCCCCN)cc2C)s1 ; CC(=O)c1c(C)c2cnc(Nc3ccc(N4CCN(CC(=O)NCCCCCCCNC(=O)c5ccc(C(=O)Nc6ncc(C)s6)c(C)c5)CC4)cn3)nc2n(C2CCCC2)c1=O]

CPD-048 was synthesized following the standard procedure for preparing CPD-008 (3.4 mg, 40% yield). MS (ESI) m/z=876.5 [M+H]⁺.

### Example 071. 4-((1-(4-(6-((6-Acetyl-8-cyclopentyl-5-methyl-7-oxo-7,8-dihydropyrido[2,3-d]pyrimidin-2-yl)(methyl)amino)pyridin-3-yl)piperazin-1-yl)-2-oxo-6,9,12,15,18-pentaoxa-3-azaicosan-20-yl)amino)-N-(4,5-dimethylthiazol-2-yl)-2-methylbenzamide (CPD-049)

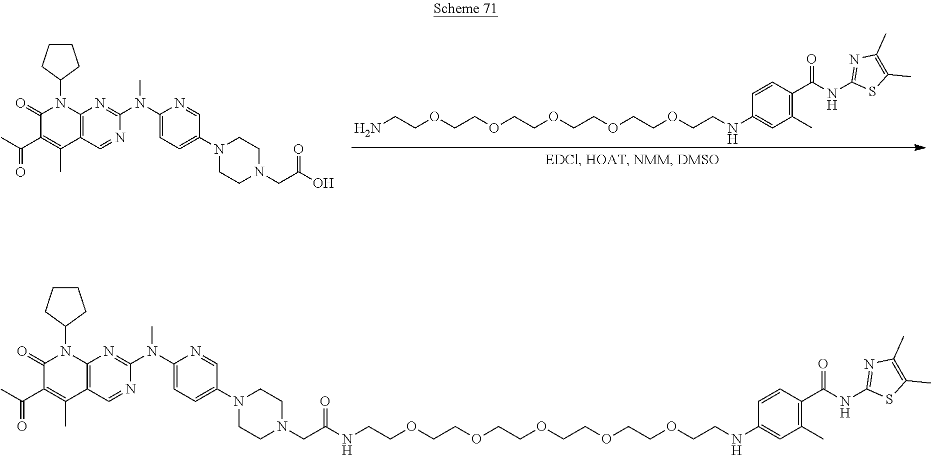 [SMILES: CC(=O)c1c(C)c2cnc(N(C)c3ccc(N4CCN(CC(=O)O)CC4)cn3)nc2n(C2CCCC2)c1=O ; Cc1cc(NCCOCCOCCOCCOCCOCCN)ccc1C(=O)Nc1nc(C)c(C)s1 ; CC(=O)c1c(C)c2cnc(N(C)c3ccc(N4CCN(CC(=O)NCCOCCOCCOCCOCCOCCNc5ccc(C(=O)Nc6nc(C)c(C)s6)c(C)c5)CC4)cn3)nc2n(C2CCCC2)c1=O]

CPD-049 was synthesized following the standard procedure for preparing CPD-042 (2.9 mg, 29% yield). MS (ESI) m/z=1026.6 [M+H]⁺.

### Example 072. 4-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-N-(1,5-dimethyl-1H-pyrazol-3-yl)-2-methylbenzamide (CPD-050)

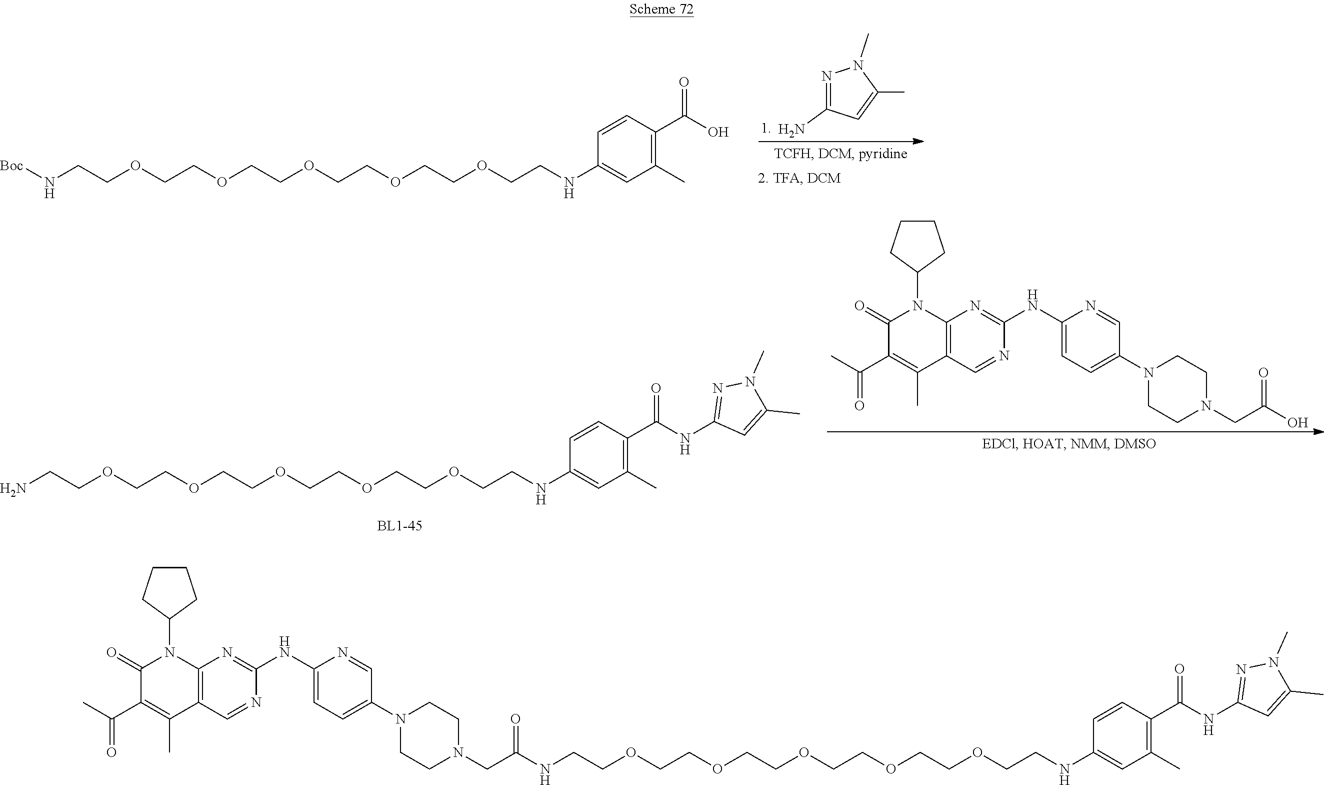

CPD-050 was synthesized following the standard procedures for preparing CPD-001 (5.3 mg, 25% yield over 3 steps). MS (ESI) m/z=995.6 [M+H]⁺.

### Example 073. N-(4,5-Dimethylthiazol-2-yl)-2-methylbenzamide (B1-1)

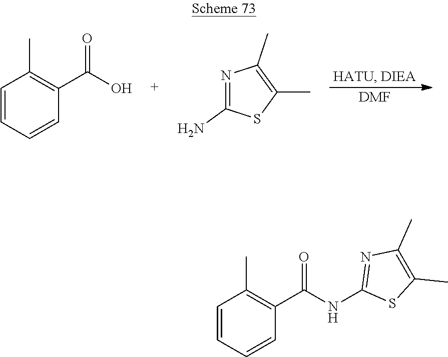

To a solution of 2-methylbenzoic acid (100 mg, 0.735 mmol) and 4,5-dimethylthiazol-2-amine (94 mg, 0.735 mmol) in DMF (3 mL) were added DIEA (190 mg, 1.47 mmol) and HATU (307 mg, 0.808 mmol) at rt. The reaction mixture was stirred at 80° C. for 1 h. After cooling down to rt, the mixture was diluted with water (10 mL) and extracted with ethyl acetate (10 mL×3). The combined organic phase was washed with brine, dried over anhydrous Na₂SO₄, filtered and concentrated under reduced pressure. The residue was purified by silica gel column chromatography to provide the desired product (48 mg, 27% yield) as a yellow solid. ¹HNMR (400 MHz, DMSO-d₆) δ 12.19 (s, 1H), 7.50 (d, J=7.6 Hz, 1H), 7.41 (t, J=7.6 Hz, 1H), 7.31-7.26 (m, 2H), 2.38 (s, 3H), 2.27 (s, 3H), 2.18 (s, 3H). MS (ESI) m/z=247.0 [M+H]⁺.

### Example 074. N-(4-Bromo-5-methylthiazol-2-yl)-2-methylbenzamide (B1-2)

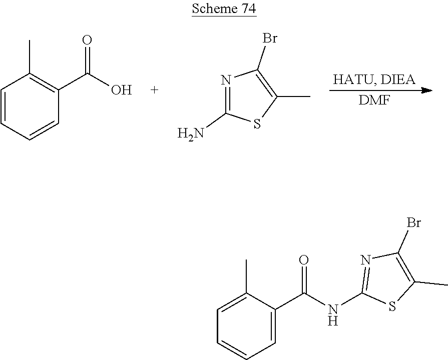

B1-2 was synthesized following the standard procedure for preparing B1-1 (111 mg, 49% yield) as a yellow solid. ¹HNMR (400 MHz, DMSO-d₆) δ 12.68 (s, 1H), 7.53 (d, J=3.6 Hz, 1H), 7.44 (t, J=7.6 Hz, 1H), 7.34-7.29 (m, 2H), 2.39 (s, 3H), 2.26 (s, 3H). MS (ESI) m/z=310.9 [M+H]⁺.

### Example 075. N-(4-Isopropyl-5-methylthiazol-2-yl)-2-methylbenzamide (B1-3)

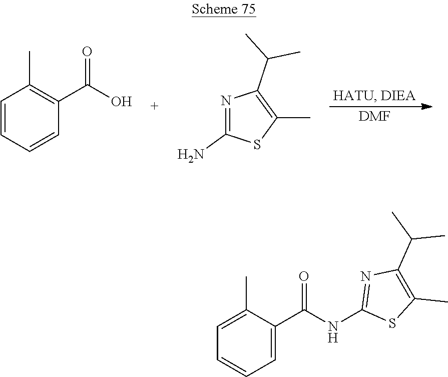

B1-3 was synthesized following the standard procedure for preparing B1-1 (19.2 mg, 32% yield) as a yellow solid. ¹HNMR (400 MHz, DMSO-d₆) δ 12.29 (brs, 1H), 7.50 (d, J=3.6 Hz, 1H), 7.39 (t, J=7.6 Hz, 1H), 7.29-7.24 (m, 2H), 3.08-3.01 (m, 1H), 2.37 (s, 3H), 2.28 (s, 3H), 1.17 (d, J=6.8 Hz, 6H). MS (ESI) m/z=275.0 [M+H]⁺.

### Example 076. Methyl 5-methyl-2-(2-methylbenzamido)thiazole-4-carboxylate (B1-4)

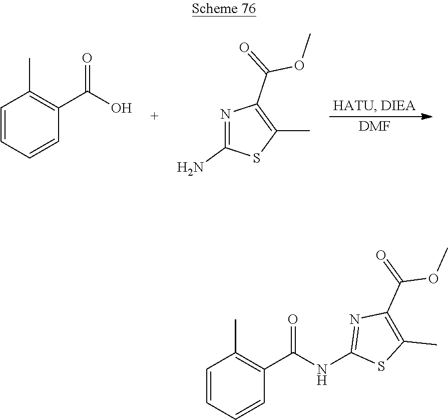

B1-4 was synthesized following the standard procedure for preparing B1-1 (100 mg, 54% yield) as a white solid. ¹HNMR (400 MHz, DMSO-d₆) δ 12.87 (s, 1H), 7.58 (d, J=7.6 Hz, 1H), 7.47-7.43 (m, 1H), 7.35-7.29 (m, 2H), 3.80 (s, 3H), 2.58 (s, 3H), 2.41 (s, 3H). MS (ESI) m/z=290.9 [M+H]⁺.

### Example 077. N-(4-Ethyl-5-methylthiazol-2-yl)-2-methylbenzamide (B1-5)

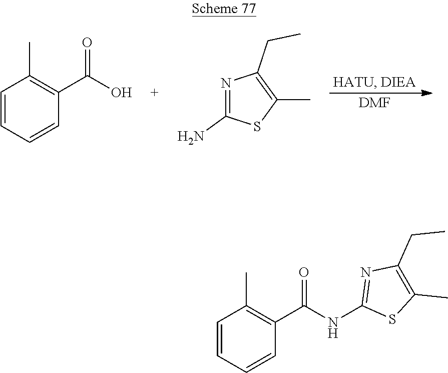

B1-5 was synthesized following the standard procedure for preparing B1-1 (130 mg, 78% yield) as a pale-white solid. ¹HNMR (400 MHz, DMSO-d₆) δ 12.22 (s, 1H), 7.49 (d, J=6.8 Hz, 1H), 7.42-7.38 (m, 1H), 7.31-7.26 (m, 2H), 2.70 (q, J=7.2 Hz, 2H), 2.38 (s, 3H), 2.19 (s, 3H), 1.19 (t, J=7.2 Hz, 3H). MS(ESI) m/z=261.0 [M+H]⁺.

### Example 078. 2-Acetamido-N-(4,5-dimethylthiazol-2-yl)benzamide (B1-6)

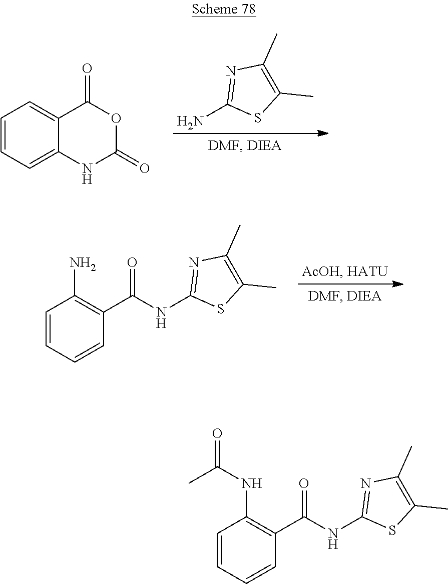

### Step 1. Synthesis of 2-amino-N-(4,5-dimethylthiazol-2-yl)benzamide

To a solution of 2H-benzo[d][1,3]oxazine-2,4(1H)-dione (400 mg, 2.45 mmol) and DIEA (632 mg, 4.90 mmol) in DMF (10 mL) was added 4,5-dimethylthiazol-2-amine (309 mg, 2.45 mmol) at rt. The reaction mixture was stirred at 80° C. for 1 h. After cooling down to rt, the mixture was diluted with water (30 mL) and extracted with ethyl acetate (20 mL×3). The combined organic phase was washed with brine, dried over anhydrous Na₂SO₄, filtered and concentrated under reduced pressure. The residue was purified by silica gel column chromatography to provide the desired product (300 mg, 50% yield) as a yellow solid. MS (ESI) m/z=248.1 [M+H]⁺.

### Step 2. Synthesis of 2-acetamido-N-(4,5-dimethylthiazol-2-yl)benzamide

A solution of 2-methylbenzoic acid (100 mg, 0.405 mmol), acetic acid (24 mg, 0.405 mmol), HATU (154 mg, 0.405 mmol), DIEA (117 mg, 0.910 mmol) in DMF (5 mL) was stirred at rt for 1 h. The reaction mixture was purified by prep-HPLC to provide the desired compound (109 mg, 32% yield) as a yellow solid. ¹HNMR (400 MHz, DMSO-d₆) 12.40 (brs, 1H), 8.14 (brs, 1H), 7.97 (brs, 1H), 7.49 (t, J=7.6 Hz, 1H), 7.15 (t, J=7.6 Hz, 1H), 2.25 (s, 3H), 2.19 (s, 3H), 2.10 (s, 3H). MS (ESI) m/z=290.1 [M+H]⁺.

### Example 079. N-(4-Cyclopropyl-5-methylthiazol-2-yl)-2-methylbenzamide (B1-7)

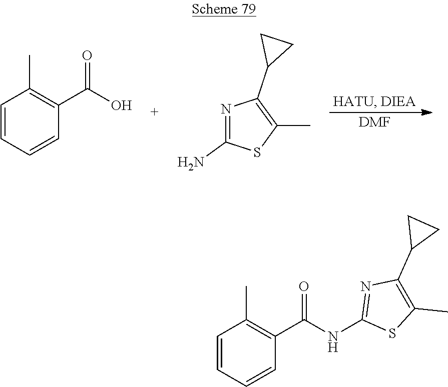

B1-7 was synthesized following the standard procedure for preparing B1-1 (72 mg, yield 46%) as a colorless oil. ¹HNMR (400 MHz, DMSO-d₆) δ 12.17 (brs, 1H), 7.48 (d, J=7.2 Hz, 1H), 7.41-7.37 (m, 1H), 7.29-7.23 (m, 2H), 2.36 (s, 3H), 2.35 (s, 3H), 1.97-1.93 (m, 1H), 0.87-0.83 (m, 2H), 0.79-0.75 (m, 2H). MS(ESI) m/z=273.0 [M+H]⁺.

### Example 080. N-(1,5-Dimethyl-1H-pyrazol-3-yl)-2-methylbenzamide (B1-8)

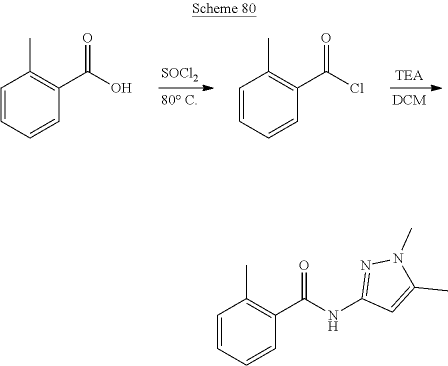

A solution of 2-methylbenzoic acid (200 mg, 1.47 mmol) in SOCl₂ (10 mL) was stirred at 80° C. for 0.5 h. After cooling down to rt, the solvent was removed under reduced pressure. The residue was dissolved in DCM (5 ml), then added to a solution of 1,5-dimethyl-1H-pyrazol-3-amine (163 mg, 1.47 mmol) and TEA (297 mg, 2.94 mmol) in DCM (10 mL) dropwise at rt. After stirring at rt for 2 h, the reaction was quenched with H₂O (5 mL) and extracted with DCM (10 mL×2). The combined organic layers were washed with brine, dried over Na₂SO₄, filtered and concentrated under reduced pressure. The resulting residue was purified by silica gel column chromatography (petroleum ether/ethyl acetate=5:1) to provide the desired product (161 mg, 48% yield) as a white solid. ¹HNMR (400 MHz, DMSO-d₆) δ 10.49 (s, 1H), 7.39-7.23 (m, 4H), 6.40 (s, 1H), 3.63 (s, 3H), 2.35 (s, 3H), 2.24 (s, 3H). MS (ESI) m/z=230.0 [M+H]⁺.

### Example 081. 2-(3-(2-(2-Aminoethoxy)ethoxy)propanamido)-N-(5-methylthiazol-2-yl)benzamide (BL1-46)

### Step 1. Synthesis of 2-nitrobenzoyl chloride

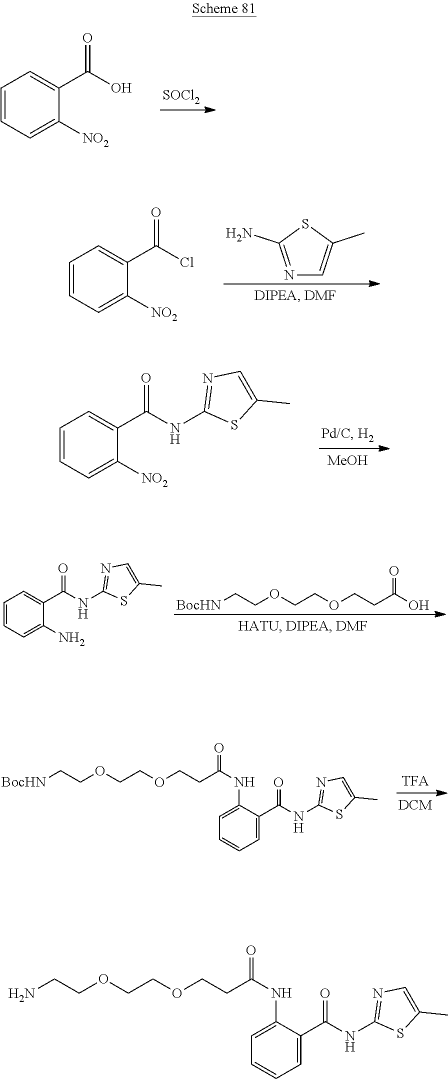

A solution of 2-nitrobenzoic acid (2 g, 0.01 mmol) in SOCl₂ (20 mL) was stirred at reflux for 2 h. The solvent was removed under reduced pressure. The resulting residue was used in the next step directly without further purification.

### Step 2. Synthesis of N-(5-methylthiazol-2-yl)-2-nitrobenzamide

To a mixture of 5-methylthiazol-2-amine (221 mg, 1.94 mmol) and DIPEA (1.04 g, 8.1 mmol) in DMF (5 mL) was added 2-nitrobenzoyl chloride (300 mg, 1.62 mmol) in DMF (5 mL) dropwise at 0° C. After the reaction mixture was stirred at rt for 30 min, it was quenched with water (50 mL) and extracted with DCM (20 mL×3). The combined organic phase was dried over anhydrous Na₂SO₄, filtered and concentrated under reduced pressure. The residue was purified by reverse-phase chromatography to provide the desired product (260 mg, 61% yield) as a colorless oil. MS (ESI) m/z=264.1 [M+H]⁺.

### Step 3. Synthesis of 2-amino-N-(5-methylthiazol-2-yl)benzamide

To a solution of N-(5-methylthiazol-2-yl)-2-nitrobenzamide (100 mg, 0.38 mmol) in MeOH (10 mL) was added 10% Pd/C (40 mg, 0.1 mmol). The reaction mixture was stirred at rt for 16 h under hydrogen balloon. The reaction was filtered through Celite and the filtrate was concentrated under reduced pressure. The resulting residue was used in the next step directly without further purification. MS (ESI) m/z=234.1 [M+H]⁺.

### Step 4. Synthesis of tert-butyl (2-(2-(3-((2-((5-methylthiazol-2-yl)carbamoyl)phenyl)amino)-3-oxopropoxy)ethoxy)ethyl)carbamate

To a mixture of 2-amino-N-(5-methylthiazol-2-yl)benzamide (60 mg, 0.25 mmol) and 2,2-dimethyl-4-oxo-3,8,11-trioxa-5-azatetradecan-14-oic acid (70 mg, 0.25 mmol) in DMF (3 mL) were added HATU (142 mg, 0.38 mmol) and DIPEA (162 mg, 1.25 mmol) at rt. After the reaction mixture was stirred at rt for 16 h, it was purified by reverse-phase chromatography to provide the desired product (45 mg, 35% yield) as a colorless oil. MS (ESI) m/z=493.3 [M+H]⁺.

### Step 5. Synthesis of 2-(3-(2-(2-aminoethoxy)ethoxy)propanamido)-N-(5-methylthiazol-2-yl)benzamide

To a solution of tert-butyl (2-(2-(3-((2-((5-methylthiazol-2-yl)carbamoyl)phenyl)amino)-3-oxopropoxy)ethoxy)ethyl)carbamate (45 mg, 0.09 mmol) in DCM (2 mL) was added TFA (1 mL) at 0° C. The reaction mixture was stirred at rt for 1 h. The solvents were removed under reduced pressure to provide the desired product (38 mg, 85% yield) as TFA salt. MS (ESI) m/z=393.3 [M+H]⁺.

### Example 082. 2-(3-(2-(2-Aminoethoxy)ethoxy)propanamido)-N-(1,5-dimethyl-1H-pyrazol-3-yl)benzamide (BL1-47)

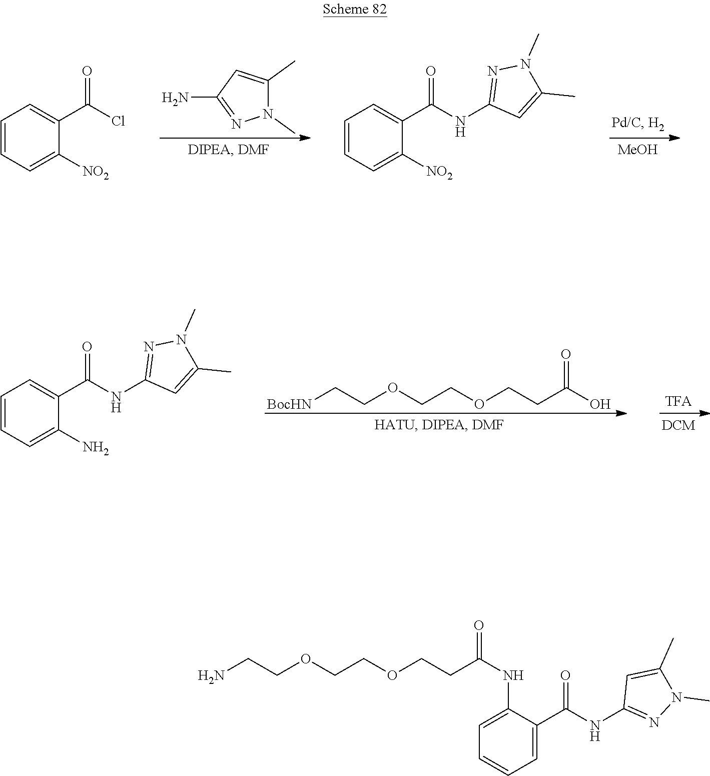

BL1-47 was synthesized following the standard procedures for preparing BL1-46 (12 mg, 36% yield over 4 steps) as TFA salt. MS (ESI) m/z=390.3 [M+H]⁺.

### Example 083. 2-(3-(2-(2-Aminoethoxy)ethoxy)propanamido)-N-(pyridin-2-yl)benzamide (BL1-48)

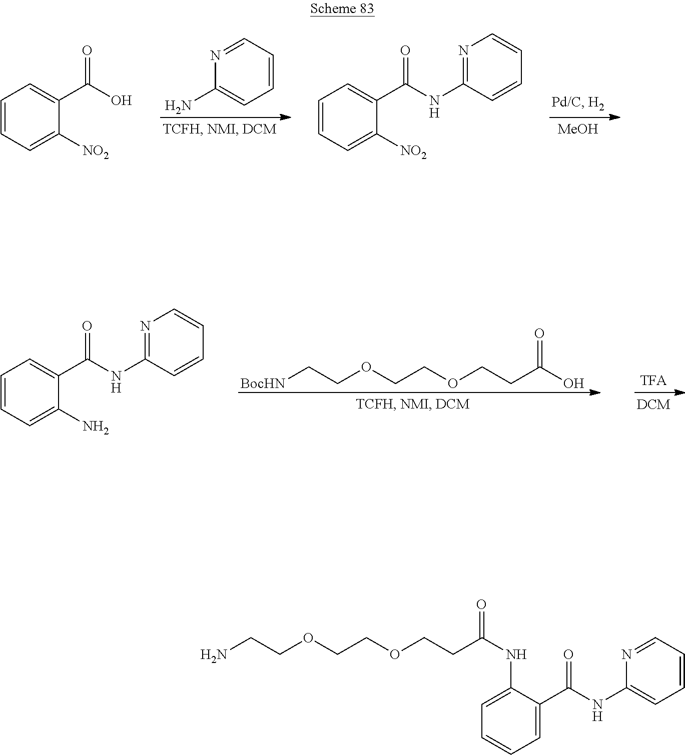

BL1-48 was synthesized following the standard procedures for preparing BL1-55 (5.0 mg, 5% yield over 4 steps) as TFA salt. MS (ESI) m/z=373.3 [M+H]⁺.

### Example 084. 2-(3-(2-(2-Aminoethoxy)ethoxy)propanamido)-N-(5-methylpyrazin-2-yl)benzamide (BL1-49)

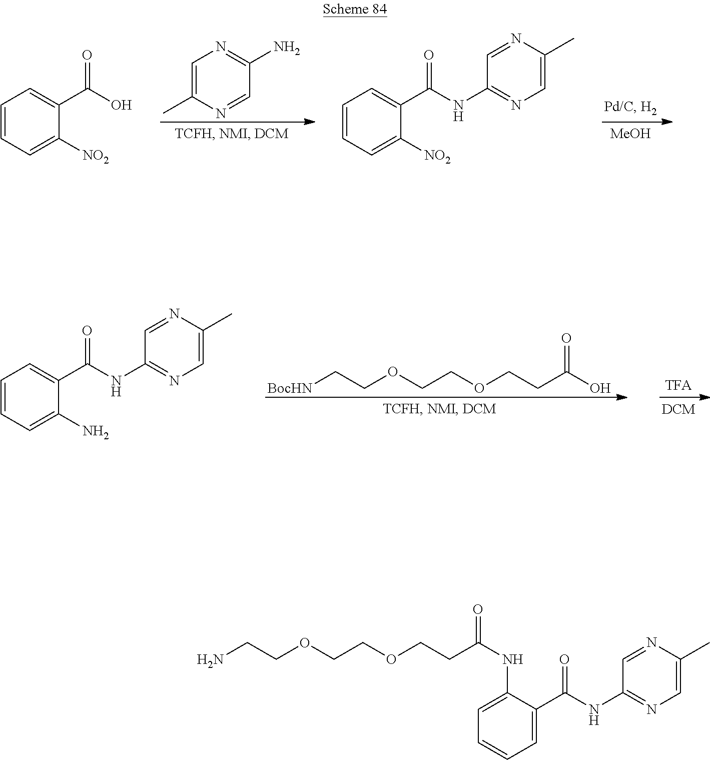

BL1-49 was synthesized following the standard procedures for preparing BL1-55 (25 mg, 12% yield over 4 steps) as TFA salt. MS (ESI) m/z=388.2 [M+H]⁺.

### Example 085. 2-(3-(2-(2-Aminoethoxy)ethoxy)propanamido)-N-(5-methylpyrimidin-2-yl)benzamide (BL1-50)

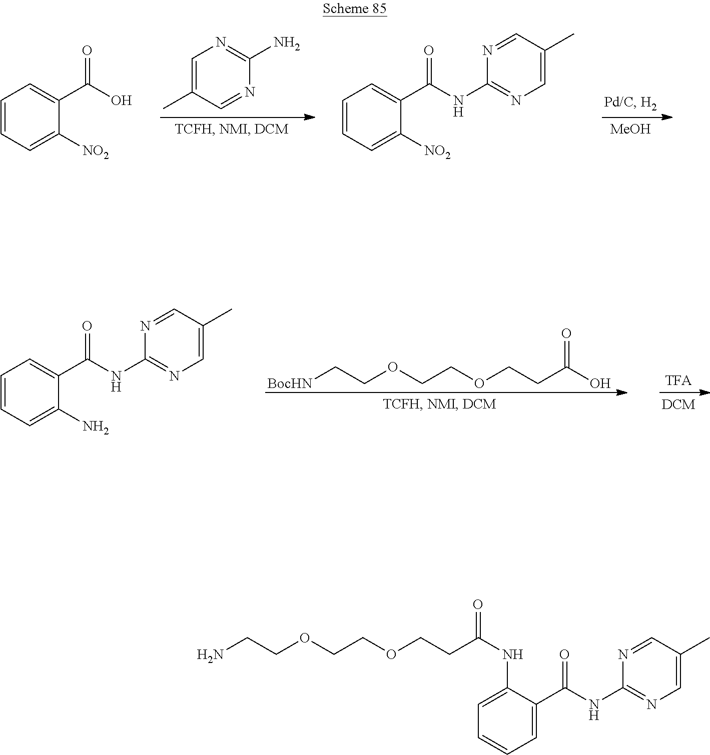

BL1-50 was synthesized following the standard procedures for preparing BL1-55 (30 mg, 17% yield over 4 steps) as TFA salt. MS (ESI) m/z=388.2 [M+H]⁺.

### Example 086. 2-(3-(2-(2-Aminoethoxy)ethoxy)propanamido)-N-(6-methylpyridazin-3-

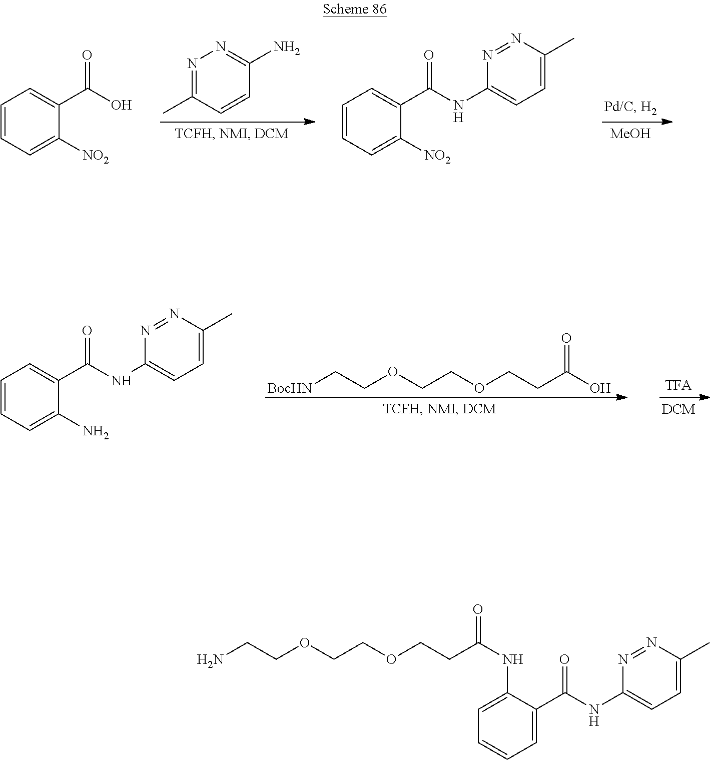

BL1-51 was synthesized following the standard procedures for preparing BL1-55 (30 mg, 24% yield over 4 steps) as TFA salt. MS (ESI) m/z=388.2 [M+H]⁺.

### Example 087. 2-(3-(2-(2-Aminoethoxy)ethoxy)propanamido)-N-(4-cyclopropyl-5-methylthiazol-2-yl)benzamide (BL1-52)

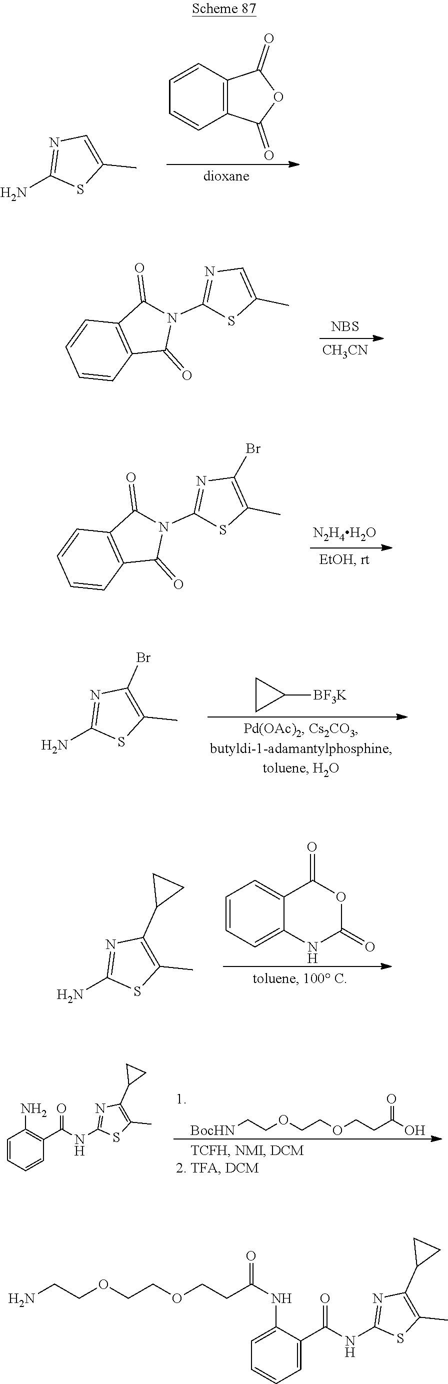

### Step 1. Synthesis of 2-(5-methylthiazol-2-yl)isoindoline-1,3-dione

A mixture of 5-methylthiazol-2-amine (10.0 g, 87.59 mmol) and isobenzofuran-1,3-dione (15.6 g, 105.1 mmol) in 1,4-dioxane (100 mL) was heated to reflux overnight. After cooling down to rt, the mixture was concentrated under reduced pressure. The residue was purified by flash chromatography (petroleum ether/ethyl acetate=10:1) to provide the desired product (9.8 g, 46% yield) as a white solid. MS (ESI) m/z=245.0 [M+H]⁺.

### Step 2. Synthesis of 2-(4-bromo-5-methylthiazol-2-yl)isoindoline-1,3-dione

To a solution of 2-(5-methylthiazol-2-yl)isoindoline-1,3-dione (2.5 g, 10.23 mmol) in CH₃CN (30 mL) was added NBS (2.2 g, 12.28 mmol) at rt. The reaction mixture was heated to 50° C. overnight. After cooling down to rt, the mixture was diluted with water (30 mL) and extracted with ethyl acetate (20 mL×3). The combined organic layers were washed with brine, dried over Na₂SO₄, filtered and concentrated under reduced pressure. The residue was purified by flash chromatography (petroleum ether/ethyl acetate=10:1) to provide the desired product (2.0 g, 61% yield) as a yellow solid. MS (ESI) m/z=323.0 [M+H]⁺.

### Step 3. Synthesis of 4-bromo-5-methylthiazol-2-amine

To a solution of 2-(4-bromo-5-methylthiazol-2-yl)isoindoline-1,3-dione (2.0 g, 6.19 mmol) in EtOH (40 mL) was added N₂H₄·H2O (1.54 g, 30.0 mmol) dropwise. After stirring at rt overnight, the mixture was filtered and concentrated under reduced pressure. The residue was purified by flash chromatography (ethyl acetate) to provide the desired product (1.0 g, 83% yield) as a white solid. MS (ESI) m/z=193.0 [M+H]⁺.

### Step 4. Synthesis of 4-cyclopropyl-5-methylthiazol-2-amine

To a solution of 4-bromo-5-methylthiazol-2-amine (1 g, 5.18 mmol) in toluene (20 mL) and H₂O (10 mL) were added potassium cyclopropyltrifluoroborate (3.83 g, 25.9 mmol), Cs₂CO₃ (5.0 g, 15.5 mmol), butyldi-1-adamantylphosphine (372 mg, 1.04 mmol) and Pd(OAc)₂ (116 mg, 0.52 mmol). The reaction mixture was stirred at 110° C. overnight. After cooling down to rt, the mixture was diluted with water (20 mL) and extracted with EtOAc (30 mL×3). The combined organic layers were dried over Na₂SO₄, filtered and concentrated under reduced pressure. The residue was purified by silica gel column chromatography (DCM/ethyl acetate=50:1) to provide the desired product (300 mg, 38% yield) as a white solid. ¹HNMR (400 MHz, DMSO-d₆) δ 6.53 (s, 2H), 2.16 (s, 3H), 1.77-1.71 (m, 1H), 0.73-0.65 (m, 4H). MS (ESI) m/z=155.1 [M+H]⁺.

### Step 5. Synthesis of 2-amino-N-(4-cyclopropyl-5-methylthiazol-2-yl)benzamide

A mixture of 4-cyclopropyl-5-methylthiazol-2-amine (300 mg, 1.93 mmol) and 1H-benzo[d][1,3]oxazine-2,4-dione (380 mg, 2.32 mmol) in toluene (15 mL) was heated at 100° C. overnight. After cooling down to rt, the reaction was concentrated under reduced pressure. The residue was purified by flash chromatography (petroleum ether/ethyl acetate=5:1) to provide the desired product (170 mg, 32% yield) as a yellow solid. ¹HNMR (400 MHz, DMSO-d₆) δ 11.91 (s, 1H), 7.79 (d, J=7.6 Hz, 1H), 7.20 (t, J=7.0 Hz, 1H), 6.75 (d, J=8.4 Hz, 1H), 6.66-6.42 (m, 3H), 2.34 (s, 3H), 1.98-1.92 (m, 1H), 0.87-0.79 (m, 4H). MS (ESI) m/z=274.0 [M+H]⁺.

### Step 6. Synthesis of tert-butyl (2-(2-(3-((2-((4-cyclopropyl-5-methylthiazol-2-yl)carbamoyl)phenyl)amino)-3-oxopropoxy)ethoxy)ethyl)carbamate

To a mixture of 2-amino-N-(4-cyclopropyl-5-methylthiazol-2-yl)benzamide (15 mg, 0.05 mmol), 2,2-dimethyl-4-oxo-3,8,11-trioxa-5-azatetradecan-14-oic acid (15 mg, 0.05 mmol) and NMI (20 mg, 0.25 mmol) in DCM (20 mL) was added TCFH (28 mg, 0.1 mmol) at 0° C. After the mixture was stirred at rt for 16 h, it was concentrated and purified by silica gel flash chromatography to provide the desired product (21 mg, 71% yield) as a colorless oil. MS (ESI) m/z=533.3 [M+H]⁺.

### Step 7. Synthesis of 2-(3-(2-(2-aminoethoxy)ethoxy)propanamido)-N-(4-cyclopropyl-5-methylthiazol-2-yl)benzamide

To a solution of tert-butyl (2-(2-(3-((2-((4-cyclopropyl-5-methylthiazol-2-yl)carbamoyl)phenyl)amino)-3-oxopropoxy)ethoxy)ethyl)carbamate (21 mg, 0.04 mmol) in DCM (2 mL) was added TFA (1 mL) at 0° C. After stirring at rt for 1 h, the reaction was concentrated under reduced pressure to provide the desired product as TFA salt (8 mg, 38% yield). MS (ESI) m/z=433.3 [M+H]⁺.

### Example 088. 2-(3-(2-(2-Aminoethoxy)ethoxy)propanamido)-N-(3-methyl-1,2,4-thiadiazol-5-yl)benzamide (BL1-53)

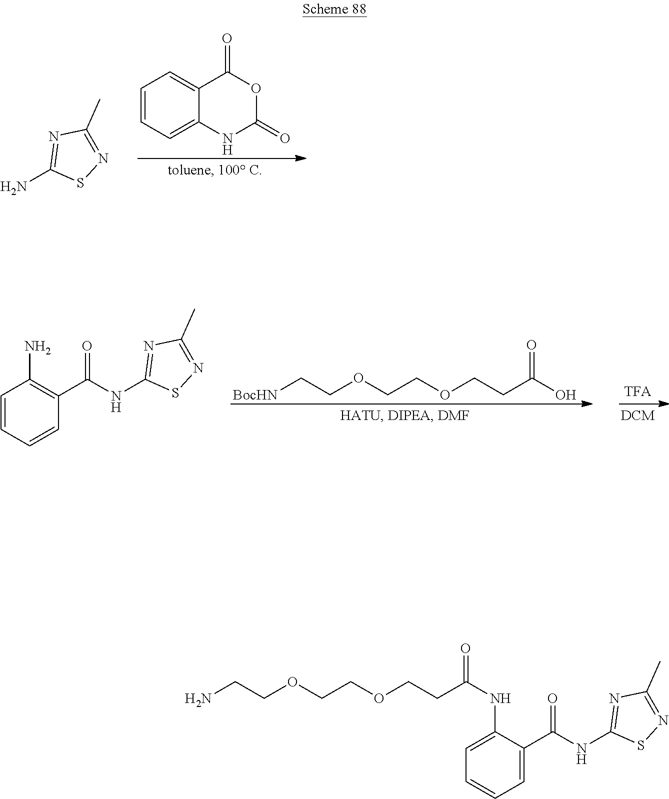

### Step 1. Synthesis of 2-amino-N-(3-methyl-1,2,4-thiadiazol-5-yl)benzamide

To a solution of 3-methyl-1,2,4-thiadiazol-5-amine (500 mg, 4.35 mmol) in toluene (10 mL) was added 1H-benzo[d][1,3]oxazine-2,4-dione (708 mg, 4.35 mmol) at rt. The reaction mixture was stirred at 100° C. overnight. After cooling down to rt, the reaction was concentrated under reduced pressure. The residue was purified by flash chromatography (petroleum ether/ethyl acetate=5:1) to provide the desired product (264 mg, 26% yield) as a white solid. ¹HNMR (400 MHz, DMSO-d₆) δ 8.73 (brs, 2H), 7.91 (dd, J=8.4 Hz, 1.2 Hz, 1H), 7.30-7.26 (m, 1H), 6.81 (dd, J=8.4 Hz, 1.2 Hz, 1H), 6.60-6.56 (m, 1H), 2.48 (s, 3H). MS (ESI) m/z=234.9 [M+H]⁺.

### Step 2. Synthesis of tert-butyl (2-(2-(3-((2-((3-methyl-1,2,4-thiadiazol-5-yl)carbamoyl)phenyl)amino)-3-oxopropoxy)ethoxy)ethyl)carbamate

To a solution of 2-amino-N-(3-methyl-1,2,4-thiadiazol-5-yl)benzamide (15 mg, 0.064 mmol) and 3-[2-[2-(tert-butoxycarbonylamino)ethoxy]ethoxy]propanoic acid (17.8 mg, 0.064 mmol) in DMF (2 mL) were added DIPEA (24.8 mg, 0.2 mmol) and HATU (37.0 mg, 0.1 mmol) at 0° C. After the reaction mixture was stirred at rt for 16 h, it was poured into water (10 mL) and extracted with EA (10 mL×3). The combined organic layers were washed with saturated brine, dried over anhydrous sodium sulfate, filtered and concentrated under reduced pressure. The resulting residue was purified by silica gel flash chromatography to provide the desired product (24 mg, 76% yield) as a yellow oil. MS (ESI) m/z=494.3 [M+H]⁺.

### Step 3. Synthesis of 2-(3-(2-(2-aminoethoxy)ethoxy)propanamido)-N-(3-methyl-1,2,4-thiadiazol-5-yl)benzamide

To a solution of tert-butyl (2-(2-(3-((2-((3-methyl-1,2,4-thiadiazol-5-yl)carbamoyl)phenyl)amino)-3-oxopropoxy)ethoxy)ethyl)carbamate (24 mg, 0.049 mmol) in DCM (2 mL) was added TFA (1 mL) at 0° C. After stirring at rt for 1 h, the reaction was concentrated under reduced pressure to provide the desired product as TFA salt (15 mg, 63% yield). MS (ESI) m/z=394.2 [M+H]⁺.

### Example 089. 2-(3-(2-(2-Aminoethoxy)ethoxy)propanamido)-N-(3-cyclopropyl-1,2,4-thiadiazol-5-yl)benzamide (BL1-54)

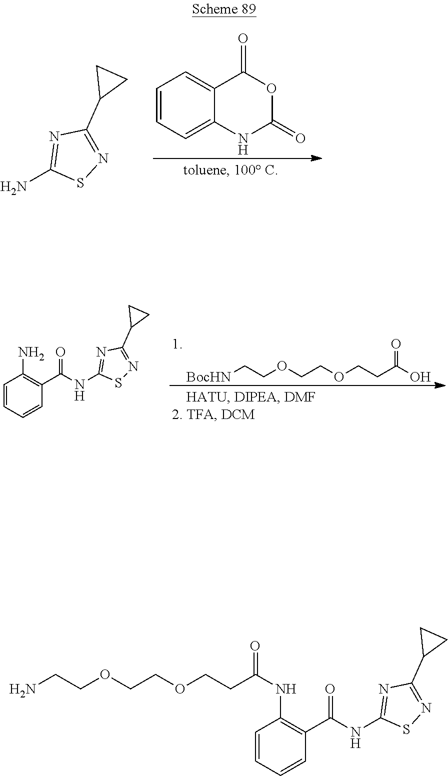

BL1-54 was synthesized following the standard procedures for preparing BL1-53 (15 mg, 62% yield over 2 steps) as TFA salt. MS (ESI) m/z=420.2 [M+H]⁺.

### Example 090. 2-(3-(2-(2-Aminoethoxy)ethoxy)propanamido)-N-(6-methylpyridin-3-yl)benzamide (BL1-55)

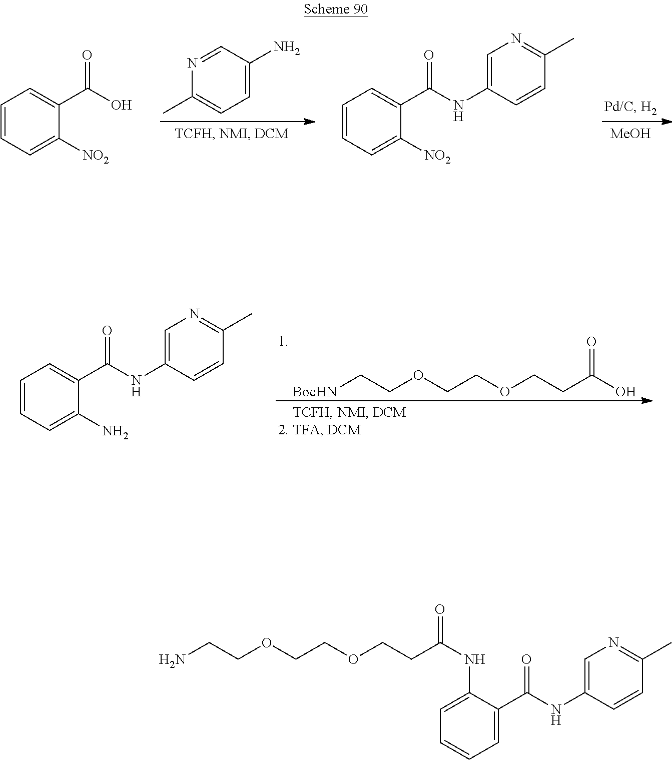

### Step 1. Synthesis of N-(6-methylpyridin-3-yl)-2-nitrobenzamide

To a mixture of 2-nitrobenzoic acid (100 mg, 0.59 mmol), 6-methylpyridin-3-amine (77.65 mg, 0.72 mmol) and NMI (242 mg, 2.95 mmol) in DCM (20 mL) was added TCFH (97 mg, 1.18 mmol) under 0° C. After the mixture was stirred at rt for 16 h, it was quenched with water (10 mL) and extracted with DCM (10 mL×2). The combined organic phase was dried over anhydrous Na₂SO₄, filtered and concentrated under reduced pressure. The residue was purified by silica gel flash chromatography to provide the desired product (132 mg, 85% yield) as a colorless oil. MS (ESI) m/z=258.4 [M+H]⁺.

### Step 2. Synthesis of 2-amino-N-(6-methylpyridin-3-yl)benzamide

To a solution of N-(6-methylpyridin-3-yl)-2-nitrobenzamide (380 mg, 0.9 mmol) in MeOH (10 mL) was added 10% Pd/C (40 mg, 0.1 mmol). The reaction mixture was stirred at rt for 16 h under hydrogen balloon. The reaction was filtered and concentrated under reduced pressure. The resulting residue was used in the next step directly without further purification. MS (ESI) m/z=228.3 [M+H]⁺.

### Step 3. Synthesis of tert-butyl (2-(2-(3-((2-((6-methylpyridin-3-yl)carbamoyl)phenyl)amino)-3-oxopropoxy)ethoxy)ethyl)carbamate

To a mixture of 2-amino-N-(6-methylpyridin-3-yl)benzamide (40 mg, 0.17 mmol), 2,2-dimethyl-4-oxo-3,8,11-trioxa-5-azatetradecan-14-oic acid (48 mg, 0.17 mmol) and NMI (69 mg, 0.85 mmol) in DCM (20 mL) was added TCFH (71 mg, 0.25 mmol) under 0° C. After the mixture was stirred at rt for 16 h, it was quenched with water (10 mL) and extracted with DCM (10 mL×2). The combined organic phase was dried over anhydrous Na₂SO₄, filtered and concentrated under reduced pressure. The residue was purified by silica gel flash chromatography to provide the desired product (55 mg, 64%) as a colorless oil. MS (ESI) m/z=487.7 [M+H]⁺.

### Step 4. Synthesis of 2-(3-(2-(2-aminoethoxy)ethoxy)propanamido)-N-(6-methylpyridin-3-yl)benzamide

To a solution of tert-butyl (2-(2-(3-((2-((6-methylpyridin-3-yl)carbamoyl)phenyl)amino)-3-oxopropoxy)ethoxy)ethyl)carbamate (50 mg, 0.1 mmol) in DCM (2 mL) was added TFA (1 mL) at 0° C. The reaction mixture was stirred at rt for 1 h. The solvents were removed under reduced pressure to provide the desired product (39.7 mg, 78% yield) as TFA salt. MS (ESI) m/z=387.3 [M+H]⁺.

### Example 091. 2-(3-(2-(2-Aminoethoxy)ethoxy)propanamido)-N-(5-methylpyridin-2-yl)benzamide (BL1-56)

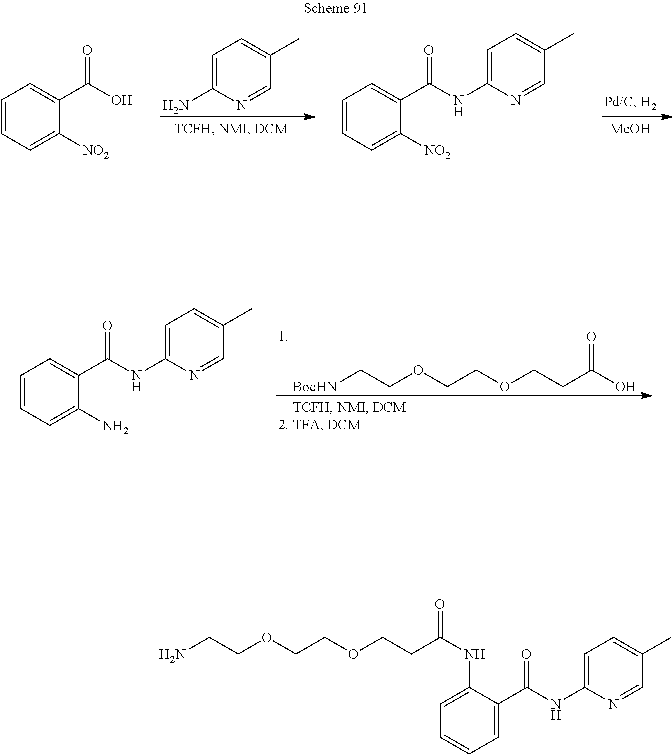

BL1-56 was synthesized following the standard procedures for preparing BL1-55 (15 mg, 18% yield over 4 steps) as TFA salt. MS (ESI) m/z=387.2 [M+H]⁺.

### Example 092. 2-(3-(2-(2-Aminoethoxy)ethoxy)propanamido)-N-(5-methyl-4-(tetrahydro-2H-pyran-4-yl)thiazol-2-yl)benzamide (BL1-57)

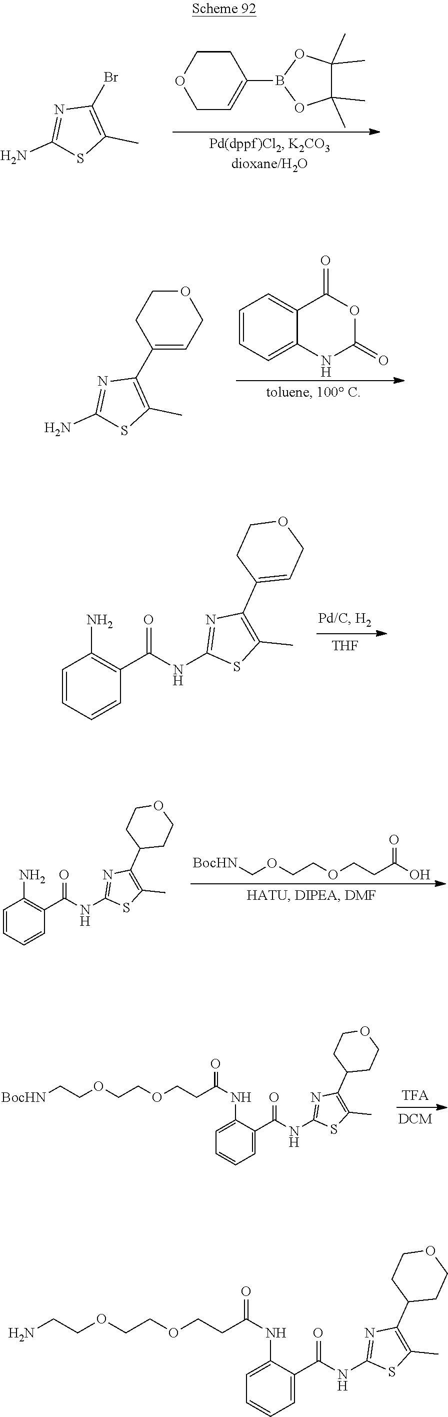

### Step 1. Synthesis of 4-(3,6-dihydro-2H-pyran-4-yl)-5-methylthiazol-2-amine

A mixture of 4-bromo-5-methylthiazol-2-amine (500 mg, 2.6 mmol), 2-(3,6-dihydro-2H-pyran-4-yl)-4,4,5,5-tetramethyl-1,3,2-dioxaborolane (1.1 g, 5.2 mmol), K₂CO₃ (900 mg, 6.5 mmol) and Pd(dppf)Cl₂ (200 mg, 0.3 mmol) in 1,4-dioxane (10 mL) and H₂O (1.0 mL) was refluxing overnight under N₂ atmosphere. After cooling down to rt, the reaction mixture was quenched with water (15 mL) and extracted with DCM (20 mL×3). The combined organic layers were dried over Na₂SO₄, filtered and concentrated under reduced pressure. The residue was purified by flash chromatography to provide the desired product (400 mg, 72% yield) as a brown oil. MS (ESI) m/z=197.0 [M+H]⁺.

### Step 2. Synthesis of 2-amino-N-(4-(3,6-dihydro-2H-pyran-4-yl)-5-methylthiazol-2-yl)benzamide

A mixture of 4-(3,6-dihydro-2H-pyran-4-yl)-5-methylthiazol-2-amine (400 mg, 2.04 mmol) and 1H-benzo[d][1,3]oxazine-2,4-dione (370 mg, 2.2 mmol) in toluene (20 mL) was heated to reflux overnight. After cooling down to rt, the reaction mixture was concentrated under reduced pressure. The residue was purified with flash chromatography (petroleum ether/ethyl acetate=1:1) to provide the desired product (200 mg, 31% yield) as a yellow solid. MS (ESI) m/z=316.1 [M+H]⁺.

### Step 3. Synthesis of 2-amino-N-(5-methyl-4-(tetrahydro-2H-pyran-4-yl)thiazol-2-yl)benzamide

To a solution of 2-amino-N-(4-(3,6-dihydro-2H-pyran-4-yl)-5-methylthiazol-2-yl)benzamide (200 mg, 0.64 mmol) in THF (20 mL) was added Pd/C (50 mg). After stirring at rt for 4 h under H₂ atmosphere, the mixture was filtered and concentrated under reduced pressure. The residue was purified by prep-HPLC to provide the desired product (72 mg, 36% yield) as a white solid. ¹HNMR (400 MHz, DMSO-d₆) δ 11.97 (s, 1H), 7.83 (d, J=7.6 Hz, 1H), 7.23-7.18 (m, 1H), 6.75 (d, J=8.0 Hz, 1H), 6.69-6.36 (m, 3H), 3.94-3.90 (m, 2H), 3.46-3.30 (m, 2H), 2.98-2.90 (m, 1H), 2.30 (s, 3H), 1.92-1.81 (m, 2H), 1.56-1.52 (m, 2H). MS (ESI) m/z=318.0 [M+H]⁺.

The remaining steps were performed according to the procedures for preparing BL1-53 to provide the desired product (10 mg, 45% yield over 2 steps) as TFA salt. MS (ESI) m/z=477.3 [M+H]⁺.

### Example 093. 2-(3-(2-(2-Aminoethoxy)ethoxy)propanamido)-N-(1-methyl-1H-imidazol-4-yl)benzamide (BL1-58)

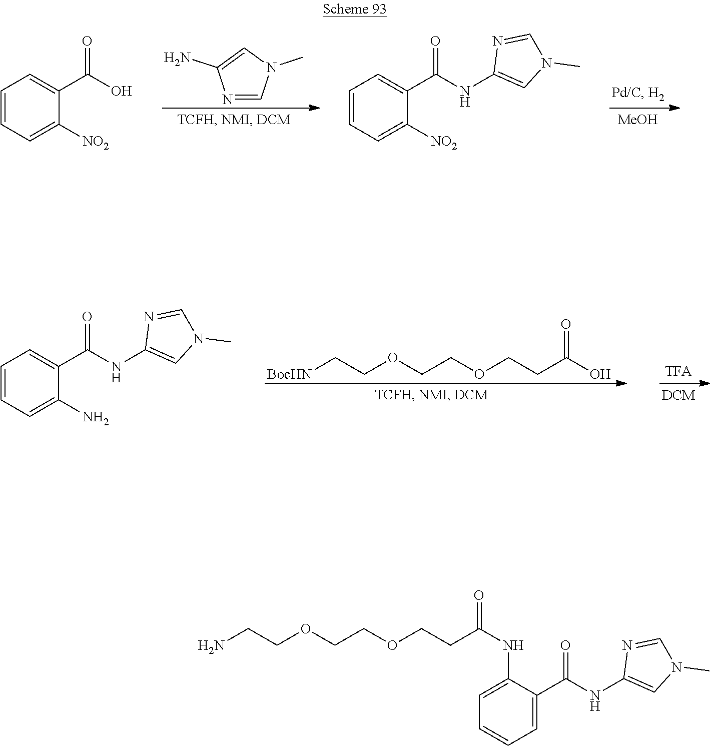

BL1-58 was synthesized following the standard procedures for preparing BL1-55 (7.0 mg, 4.8% yield over 4 steps) as TFA salt. MS (ESI) m/z=376.2 [M+H]⁺.

### Example 094. 2-(3-(2-(2-Aminoethoxy)ethoxy)propanamido)-N-(5-methyl-1H-imidazol-2-yl)benzamide (BL1-59)

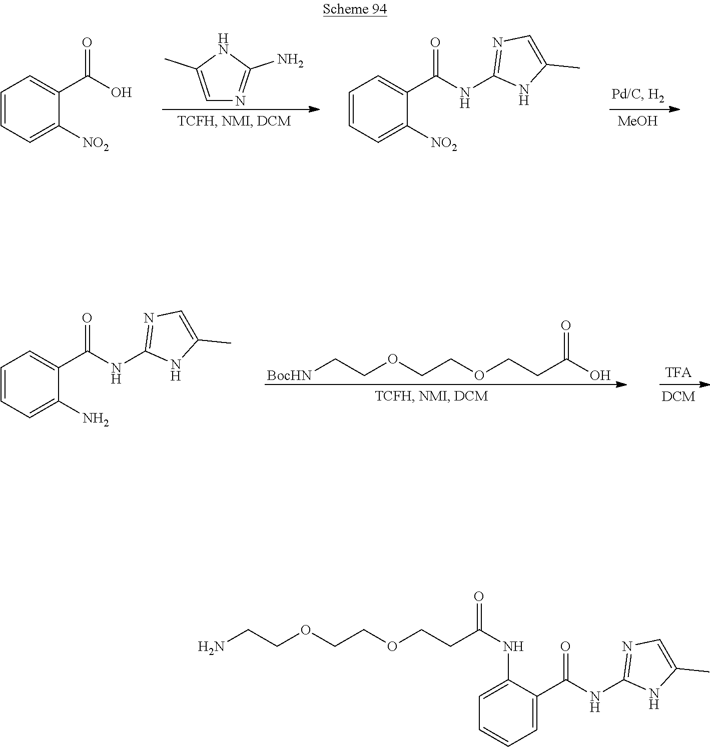

BL1-59 was synthesized following the standard procedures for preparing BL1-55 (12.0 mg, 5.4% yield over 4 steps) as TFA salt. MS (ESI) m/z=376.2 [M+H]⁺.

### Example 095. 2-(3-(2-(2-Aminoethoxy)ethoxy)propanamido)-N-(5-methylthiophen-2-yl)benzamide (BL1-60)

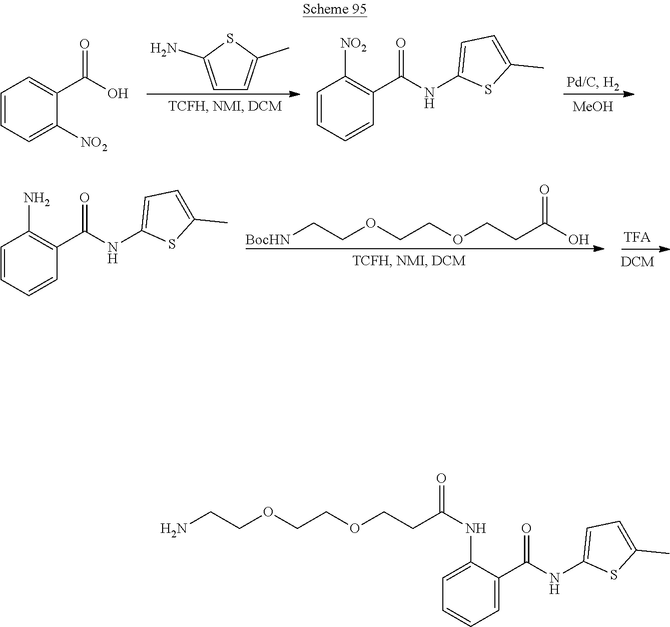

BL1-60 was synthesized following the standard procedures for preparing BL1-55 (12.0 mg, 14% yield over 4 steps) as TFA salt. MS (ESI) m/z=392.2 [M+H]⁺.

### Example 096. 2-(3-(2-(2-Aminoethoxy)ethoxy)propanamido)-N-(5-methyloxazol-2-yl)benzamide (BL1-61)

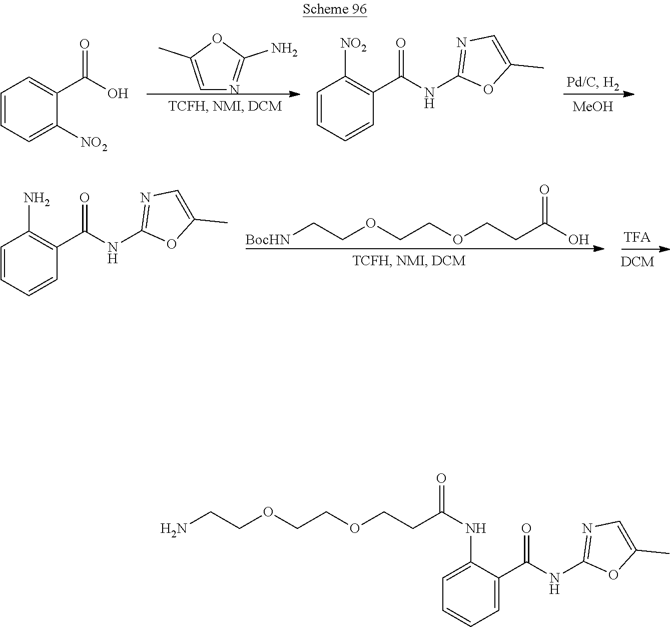

BL1-61 was synthesized following the standard procedures for preparing BL1-55 (15.0 mg, 37% yield over 4 steps) as TFA salt. MS (ESI) m/z=377.2 [M+H]⁺.

### Example 097. 3-((2-(2-(2-Aminoethoxy)ethoxy)ethyl)amino)-N-(1,5-dimethyl-1H-pyrazol-3-yl)-2-methylbenzamide (BL1-62)

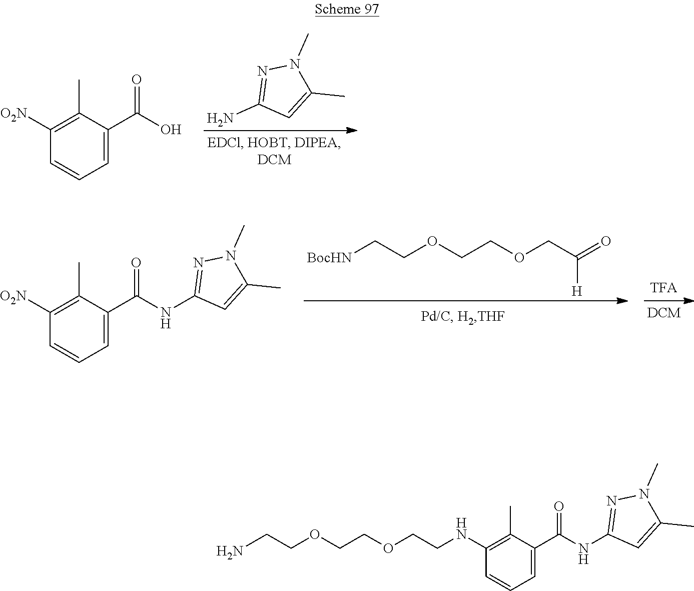

### Step 1. Synthesis of N-(1,5-dimethyl-1H-pyrazol-3-yl)-2-methyl-3-nitrobenzamide

To a solution of 2-methyl-3-nitrobenzoic acid (400 mg, 2.2 mmol) and 1,5-dimethyl-1H-pyrazol-3-amine (269 mg, 2.42 mmol) in DCM (20 mL) were added EDCI (1.26 g, 6.6 mmol), HOBt (446 mg, 3.3 mmol) and DIEA (851 mg, 6.6 mmol) at rt. After stirring at rt overnight, the reaction was quenched with water (10 mL) and extracted with DCM (20 mL×3). The combined organic layers were dried over Na₂SO₄, filtered and concentrated under reduced pressure. The residue was purified by flash chromatography (DCM/ethyl acetate=4:1) to provide the desired product (900 mg, 91% yield) as a white solid. MS (ESI) m/z=275.2 [M+H]⁺.

### Step 2. Synthesis of tert-butyl (2-(2-(2-((3-((1,5-dimethyl-1H-pyrazol-3-yl)carbamoyl)-2-methylphenyl)amino)ethoxy)ethoxy)ethyl)carbamate

To a stirred solution of N-(1,5-dimethyl-1H-pyrazol-3-yl)-2-methyl-3-nitrobenzamide (550 mg, 2.0 mmol) in THF (10 mL) was added tert-butyl (2-(2-(2-oxoethoxy)ethoxy)ethyl)carbamate (744 mg, 3.0 mmol) and Pd/C (110 mg) under N₂. The suspension was degassed under vacuum and purged with H₂ several times. After stirring at rt under hydrogen balloon overnight, the mixture was filtered through a pad of Celite and the filter cake was washed with MeOH. The filtrate was concentrated under reduced pressure. The residue was purified by silica gel column chromatography (DCM/MeOH=20:1) to provide the desired product (100 mg, 11% yield) as a white solid. ¹HNMR (400 MHz, DMSO-d₆) δ 10.34 (s, 1H), 7.06 (t, J=7.6 Hz, 1H), 6.74-6.72 (m, 1H), 6.65 (d, J=8.0 Hz, 1H), 6.60 (d, J=8.0 Hz, 1H), 6.38 (s, 1H), 4.85 (t, J=5.6 Hz, 1H), 3.62-3.59 (m, 5H), 3.53 (d, J=4.4 Hz, 4H), 3.41-3.38 (m, 2H), 3.28 (q, J=5.6 Hz, 2H), 3.07 (q, J=5.6 Hz, 2H), 2.23 (s, 3H), 2.04 (s, 3H), 1.37 (s, 9H). MS (ESI) m/z=476.1 [M+H]⁺.

### Step 3. Synthesis of 3-((2-(2-(2-aminoethoxy)ethoxy)ethyl)amino)-N-(1,5-dimethyl-1H-pyrazol-3-yl)-2-methylbenzamide

To a solution of tert-butyl (2-(2-(2-((3-((1,5-dimethyl-1H-pyrazol-3-yl)carbamoyl)-2-methylphenyl)amino)ethoxy)ethoxy)ethyl)carbamate (10 mg, 0.021 mmol) in DCM (2 mL) was added TFA (1 mL) at rt. After stirring at rt for 1 h, the reaction mixture was concentrated under reduced pressure to provide the desired product (8 mg, 80% yield) as TFA salt. MS (ESI) m/z=376.3 [M+H]⁺.

### Example 098. 2-(3-(2-(2-Aminoethoxy)ethoxy)propanamido)-N-(1-methyl-1H-pyrazol-3-yl)benzamide (BL1-63)

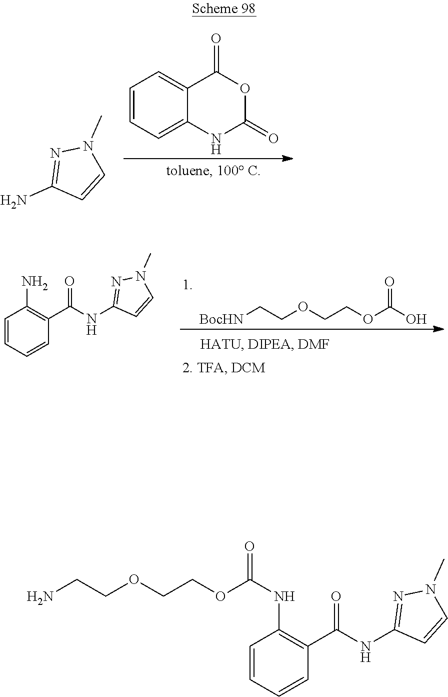

BL1-63 was synthesized following the standard procedures for preparing BL1-53 (8.0 mg, 14% yield over 3 steps) as TFA salt. MS (ESI) m/z=376.2 [M+H]⁺.

### Example 099. 2-(3-(2-(2-Aminoethoxy)ethoxy)propanamido)-N-(1-methyl-5-(trifluoromethyl)-1H-pyrazol-3-yl)benzamide (BL1-64)

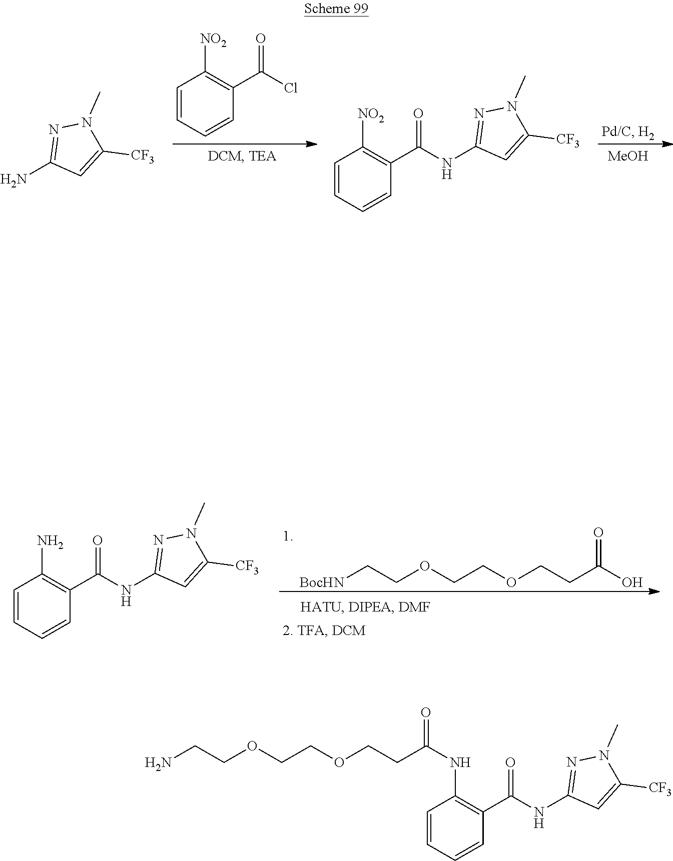

### Step 1. Synthesis of N-(1-methyl-5-(trifluoromethyl)-1H-pyrazol-3-yl)-2-nitrobenzamide

To a solution of 1-methyl-5-(trifluoromethyl)-1H-pyrazol-3-amine (500 mg, 3.0 mmol) in DCM (10 mL) were added TEA (612 mg, 6.0 mmol) and 2-nitrobenzoyl chloride (666 mg, 3.6 mmol). After the mixture was stirred at rt for 2 h, it was quenched with H₂O (10 mL) and extracted with DCM (10 mL×3). The combined organic layers were washed with brine, dried over Na₂SO₄, filtered and concentrated under reduced pressure. The residue was purified by silica gel column chromatography (petroleum ether/ethyl acetate=5:1) to provide the desired product (587 mg, 62% yield) as a white solid. MS (ESI) m/z=315.1 [M+H]⁺.

The remaining steps were performed according to the procedures for preparing BL1-46 to provide the desired product (8.0 mg, 22% yield over 3 steps) as TFA salt. MS (ESI) m/z=444.3 [M+H]⁺.

### Example 100. 2-(3-(2-(2-Aminoethoxy)ethoxy)propanamido)-N-(4-isopropyl-5-methylthiazol-2-yl)benzamide (BL1-65)

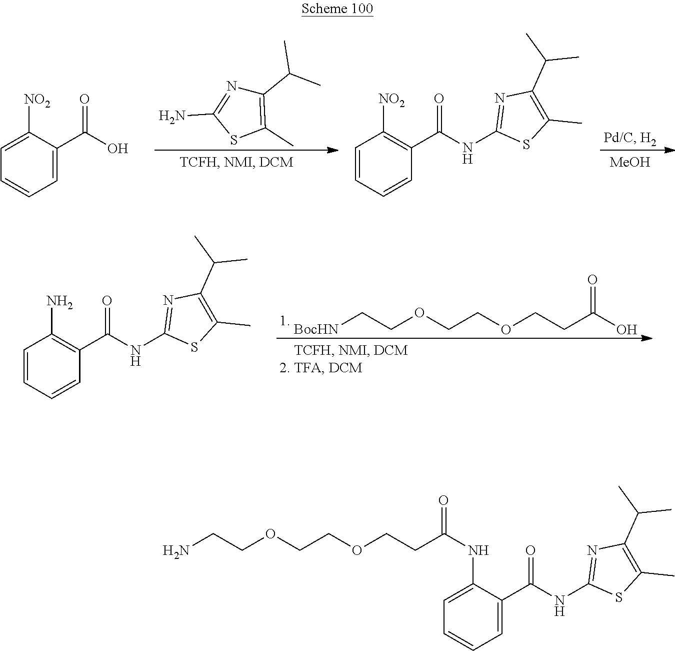

BL1-65 was synthesized following the standard procedures for preparing BL1-55 (30 mg, 23% yield over 4 steps) as TFA salt. MS (ESI) m/z=435.2 [M+H]⁺.

### Example 101. 2-(3-(2-(2-Aminoethoxy)ethoxy)propanamido)-N-(4-bromo-5-methylthiazol-2-yl)benzamide (BL1-66)

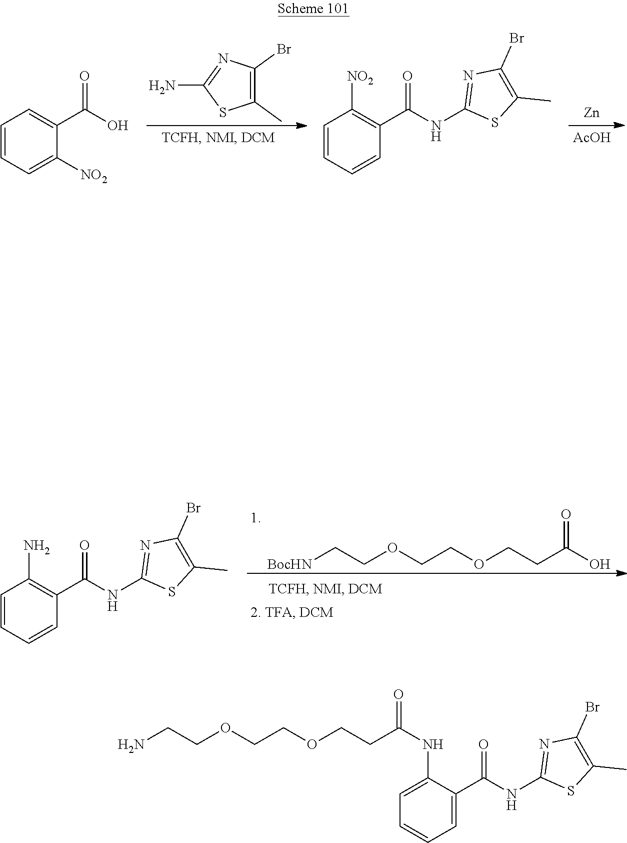

### Step 1. Synthesis of N-(4-bromo-5-methylthiazol-2-yl)-2-nitrobenzamide

To a mixture of 2-nitrobenzoic acid (50 mg, 0.30 mmol) and 4-bromo-5-methyl-thiazol-2-amine (57.7 mg, 0.30 mmol) in DCM (5 mL) were added NMI (122.6 mg, 1.50 mmol) and TCFH (16.1 mg, 0.45 mmol) at 0° C. After the reaction mixture was stirred at rt for 16 h, it was concentrated and purified by reverse-phase chromatography to provide the desired product (65 mg, yield 63%) as a colorless oil. MS (ESI) m/z=342.0 [M+H]⁺.

### Step 2. Synthesis of 2-amino-N-(4-bromo-5-methylthiazol-2-yl)benzamide

To a mixture of N-(4-bromo-5-methyl-thiazol-2-yl)-2-nitro-benzamide (65 mg, 0.19 mmol) in AcOH (10 mL) was added zinc powder (124 mg, 1.90 mmol). The reaction mixture was stirred at 60° C. for 3 h. After cooling down to rt, the mixture was filtered through Celite. The filtrate was concentrated and purified by reverse-phase chromatography to provide the desired product (56 mg, yield 94%) as a white solid. MS (ESI) m/z=312.1 [M+H]⁺.

The remaining steps were performed according to the procedures for preparing BL1-55 to provide the desired product (50 mg, 59% yield over 2 steps) as TFA salt. MS (ESI) m/z=471.1 [M+H]⁺.

### Example 102. Tert-Butyl 4-(2-(2-(3-(2-(2-aminoethoxy)ethoxy)propanamido)benzamido)-5-methylthiazol-4-yl)piperidine-1-carboxylate (BL1-67)

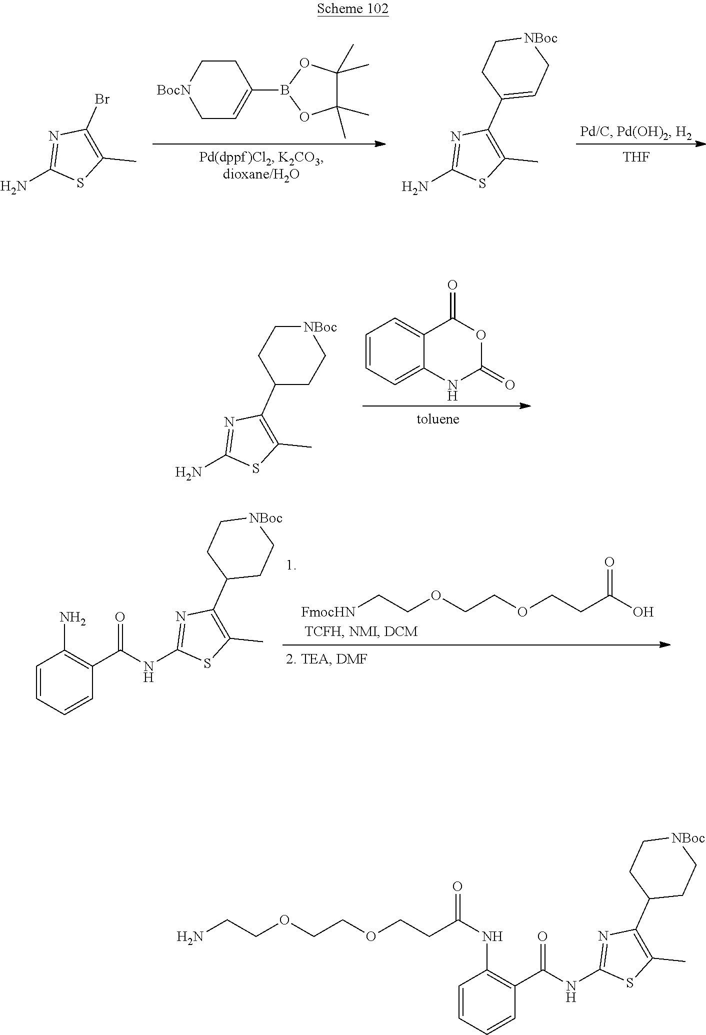

### Step 1. Synthesis of tert-butyl 4-(2-amino-5-methylthiazol-4-yl)-3,6-dihydropyridine-1(2H)-carboxylate

To a solution of 4-bromo-5-methylthiazol-2-amine (600 mg, 3.11 mmol) in 1,4-dioxane (10 mL) and H₂O (1.0 mL) were added tert-butyl 4-(4,4,5,5-tetramethyl-1,3,2-dioxaborolan-2-yl)-5,6-dihydropyridine-1(2H)-carboxylate (2.0 g, 6.22 mmol), K₂CO₃ (1.08 g, 7.7 mmol) and Pd(dppf)Cl₂ (285 mg, 0.4 mmol). The reaction mixture was stirred at reflux overnight. After cooling down to rt, the reaction was quenched with water (10 mL) and extracted with ethyl acetate (10 mL×3). The combined organic layers were washed with brine, dried over Na₂SO₄, filtered and concentrated under reduced pressure. The residue was purified by silica gel column chromatography (petroleum ether/ethyl acetate=5:1) to provide the desired product (600 mg, 65% yield) as a brown oil. MS (ESI) m/z=296.0 [M+H]⁺.

### Step 2. Synthesis of tert-butyl 4-(2-amino-5-methylthiazol-4-yl)piperidine-1-carboxylate

To a solution of tert-butyl 4-(2-amino-5-methylthiazol-4-yl)-5,6-dihydropyridine-1(2H)-carboxylate (300 mg, 1.01 mmol) in THF (20 mL) was added Pd/C (50 mg) and Pd(OH)₂ (50 mg) at rt. After stirring at rt for 4 h under H₂ atmosphere, the reaction mixture was filtered and concentrated to provide the crude product (200 mg) which was used directly in the next step without further purification. MS (ESI) m/z=298.0 [M+H]⁺.

### Step 3. Synthesis of tert-butyl 4-(2-(2-aminobenzamido)-5-methylthiazol-4-yl)piperidine-1-carboxylate

A mixture of tert-butyl 4-(2-amino-5-methylthiazol-4-yl)piperidine-1-carboxylate (200 mg, 0.67 mmol) and 1H-benzo[d][1,3]oxazine-2,4-dione (132 mg, 0.81 mmol) in toluene (20 mL) was stirred at reflux overnight. After cooling down to rt, the reaction mixture was concentrated under reduced pressure. The residue was purified by flash chromatography (petroleum ether/ethyl acetate=5:1) to provide the desired product (70 mg, 25% yield) as a yellow solid. ¹HNMR (400 MHz, DMSO-d₆) δ 11.92 (s, 1H), 7.80 (d, J=8.0 Hz, 1H), 7.20 (t, J=7.2 Hz, 1H), 6.75 (d, J=8.4 Hz, 1H), 6.66-6.45 (m, 3H), 4.05-4.00 (m, 2H), 2.92-2.85 (m, 3H), 2.29 (s, 3H), 1.72-1.59 (m, 4H), 1.41 (s, 9H). MS (ESI) m/z=417.0 [M+H]⁺.

### Step 4. Synthesis of tert-butyl 4-(2-(2-(1-(9H-fluoren-9-yl)-3-oxo-2,7,10-trioxa-4-azatridecan-13-amido)benzamido)-5-methylthiazol-4-yl)piperidine-1-carboxylate

To a mixture of tert-butyl 4-(2-(2-aminobenzamido)-5-methylthiazol-4-yl)piperidine-1-carboxylate (20 mg, 0.05 mmol) and 1-(9H-fluoren-9-yl)-3-oxo-2,7,10-trioxa-4-azatridecan-13-oic acid (19 mg, 0.05 mmol) in DCM (20 mL) were added NMI (12 mg, 0.15 mmol) and TCFH (21 mg, 0.075 mmol) at 0° C. After stirring at rt for 16 h, the reaction mixture was concentrated and purified by silica gel flash chromatography to provide the desired product (32 mg, 73% yield) as a colorless oil. MS (ESI) m/z=798.4 [M+H]⁺.

### Step 5. Synthesis of tert-butyl 4-(2-(2-(3-(2-(2-aminoethoxy)ethoxy)propanamido)benzamido)-5-methylthiazol-4-yl)piperidine-1-carboxylate

To a solution of tert-butyl 4-(2-(2-(1-(9H-fluoren-9-yl)-3-oxo-2,7,10-trioxa-4-azatridecan-13-amido)benzamido)-5-methylthiazol-4-yl)piperidine-1-carboxylate (32 mg, 0.04 mmol) in DMF (3 mL) was added TEA (41 mg, 0.4 mmol) at rt. After stirring at rt for 16 h, the reaction mixture was purified by reverse-phase chromatography to provide the desired product (20 mg, 86% yield) as a white solid. MS (ESI) m/z=576.3 [M+H]⁺.

### Example 103. 2-(3-(2-(2-Aminoethoxy)ethoxy)propanamido)-N-(1H-pyrazol-3-yl)benzamide (BL1-68)

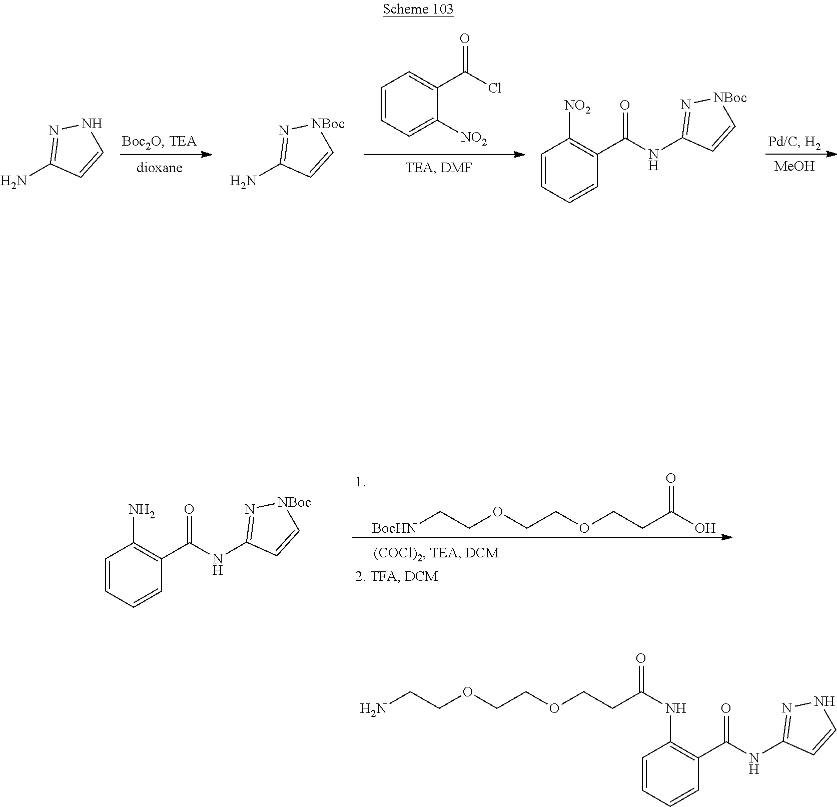

### Step 1. Synthesis of tert-butyl 3-amino-1H-pyrazole-1-carboxylate

To a solution of 1H-pyrazol-3-amine (2.0 g, 24 mmol) in 1,4-dioxane (50 mL) were added Boc₂O (6.4 g, 29 mmol) and TEA (4.3 g, 48 mmol). After the reaction mixture was stirred at rt for 12 h, it was diluted with EtOAc (100 mL) and washed with sat. NH₄Cl (30 mL×3). The organic phase was dried over Na₂SO₄, filtered and concentrated under reduced pressure. The residue was purified by silica gel flash chromatography (petroleum ether/ethyl acetate=2:1) to provide the desired product (1.2 g, 27% yield) as a yellow solid. MS (ESI) m/z=184.1 [M+H]⁺.

### Step 2. Synthesis of tert-butyl 3-(2-nitrobenzamido)-1H-pyrazole-1-carboxylate

The title compound was synthesized following the standard procedure for preparing BL1-46 (380 mg, 21% yield) as a white solid. MS (ESI) m/z=333.1 [M+H]⁺.

### Step 3. Synthesis of tert-butyl 3-(2-aminobenzamido)-1H-pyrazole-1-carboxylate

The title compound was synthesized following the standard procedure for preparing BL1-46 (160 mg, 63% yield) as a white solid. MS (ESI) m/z=302.1 [M+H]⁺.

### Step 4. Synthesis of tert-butyl 3-(2-(2,2-dimethyl-4-oxo-3,8,11-trioxa-5-azatetradecan-14-amido)benzamido)-1H-pyrazole-1-carboxylate

To a solution of 2,2-dimethyl-4-oxo-3,8,11-trioxa-5-azatetradecan-14-oic acid (140 mg, 0.50 mmol) in DCM (5 mL) were added (COCl)₂ (64 mg, 0.50 mmol) dropwise and one drop of DMF at 0° C. After stirring at 0° C. for 2 h, the mixture was added to a solution of tert-butyl 3-(2-aminobenzamido)-1H-pyrazole-1-carboxylate (100 mg, 0.33 mmol) in DCM (5 mL) dropwise at 0° C. The reaction mixture was stirred at rt for another 2 h, before it was quenched with water (5 mL) and extracted with DCM (10 mL×2). The combined organic layers were washed with brine, dried over Na₂SO₄, filtered and concentrated under reduced pressure. The residue was purified by silica gel flash chromatography (petroleum ether/ethyl acetate=1:1) to provide the desired product (65 mg, 39% yield) as a pale-yellow solid. ¹HNMR (400 MHz, DMSO-d) b 11.46 (s, 1H), 10.58 (s, 1H), 8.24 (d, J=2.8 Hz, 1H), 8.20 (d, J=8.4 Hz, 1H), 7.84-7.81 (m, 1H), 7.54-7.49 (m, 1H), 7.19-7.15 (m, 1H), 6.95 (d, J=2.8 Hz, 1H), 6.70 (t, J=5.0 Hz, 1H), 3.68 (t, J=6.2 Hz, 2H), 3.53-3.45 (m, 4H), 3.34-3.31 (m, 2H), 3.05-2.99 (m, 2H), 2.57 (t, J=6.0 Hz, 2H), 1.58 (s, 9H), 1.38 (s, 9H). MS (ESI) m/z=562.4 [M+H]⁺.

### Step 5. Synthesis of 2-(3-(2-(2-aminoethoxy)ethoxy)propanamido)-N-(1H-pyrazol-3-yl)benzamide

The title compound was synthesized following the standard procedure for preparing BL1-46 (6.0 mg, 93% yield) as TFA salt. MS (ESI) m/z=362.2 [M+H]⁺.

### Example 104. 2-(3-(2-(2-Aminoethoxy)ethoxy)propanamido)-N-(5-methyl-1H-pyrazol-3-yl)benzamide (BL1-69)

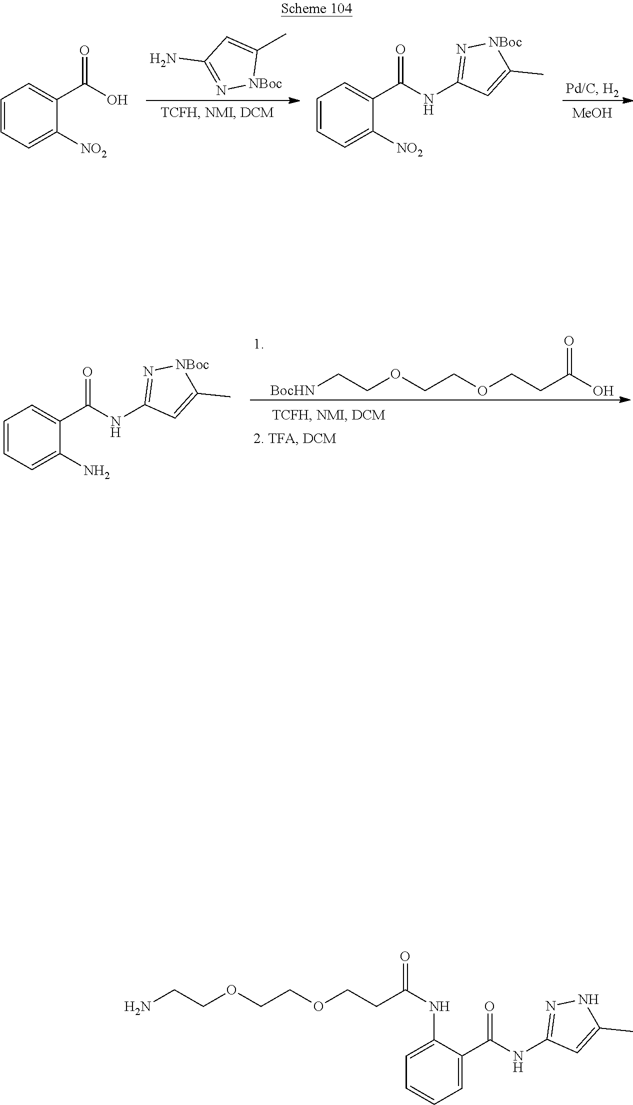

BL1-69 was synthesized following the standard procedures for preparing BL1-55 (20 mg, 45% yield over 4 steps) as TFA salt. MS (ESI) m/z=376.2 [M+H]⁺.

### Example 105. 2-(3-(2-(2-Aminoethoxy)ethoxy)propanamido)-N-(4-ethyl-5-methylthiazol-2-yl)benzamide (BL1-70)

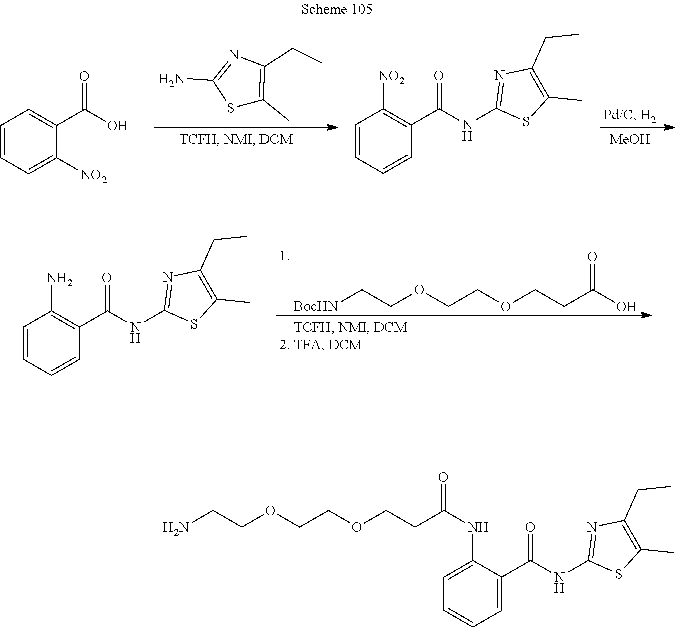

BL1-70 was synthesized following the standard procedures for preparing BL1-55 (30 mg, 49% yield over 4 steps) as TFA salt. MS (ESI) m/z=421.2 [M+H]⁺.

### Example 106. 2-(3-(2-(2-Aminoethoxy)ethoxy)propanamido)-N-(1-isopropyl-5-methyl-1H-pyrazol-3-yl)benzamide (BL1-71)

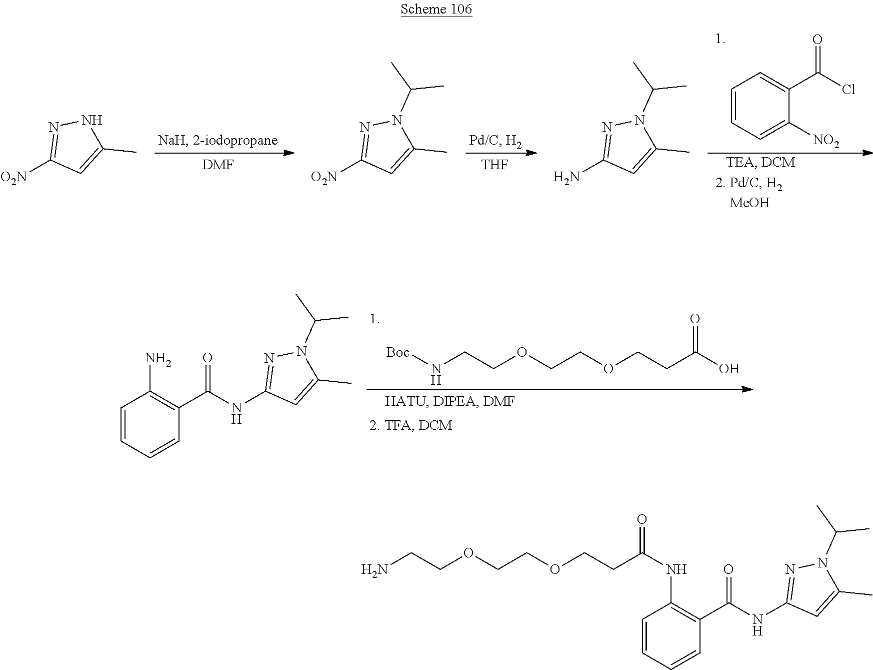

### Step 1. Synthesis of 1-isopropyl-5-methyl-3-nitro-1H-pyrazole

To a solution of 5-methyl-3-nitro-1H-pyrazole (2.0 g, 15.7 mmol) in DMF (20 mL) was added NaH (940 mg, 23.6 mmol) at 0° C. After the mixture was stirred at 0° C. for 1 h, 2-iodopropane (5.36 g, 31.5 mmol) was added dropwise at 0° C. The mixture was warmed to rt and stirred for another 8 h. The mixture was diluted with EtOAc (50 mL), washed with sat. NH₄Cl (20 mL×2) and 1 N LiCi (10 mL×2). The organic layer was concentrated under reduced pressure. The residue was purified by silica gel column chromatography (petroleum/ethyl acetate=10:1) to provide the crude product (1.6 g, 60% yield) as a yellow oil. MS (ESI) m/z=170.1 [M+H]⁺.

### Step 2. Synthesis of 1-isopropyl-5-methyl-1H-pyrazol-3-amine

To a stirred solution of 1-isopropyl-5-methyl-3-nitro-1H-pyrazole (1.6 g, 9.47 mmol) in THF (50 mL) was added Pd/C (320 mg) under N₂. The suspension was degassed under vacuum and purged with hydrogen several times. After stirring at rt overnight under hydrogen balloon, the mixture was filtered through a pad of Celite and the filter cake was washed with MeOH. The filtrate was concentrated under reduced pressure. The residue was purified by silica gel column chromatography (DCM/MeOH=20:1) to provide the desired product (1.6 g, crude) as a white solid. MS (ESI) m/z=140.2 [M+H]⁺.

### Step 3. Synthesis of N-(1-isopropyl-5-methyl-1H-pyrazol-3-yl)-2-nitrobenzamide

To a stirred solution of 1-isopropyl-5-methyl-1H-pyrazol-3-amine (800 mg, 5.7 mmol) and Et₃N (2.3 mL, 17.1 mmol) in DCM (5 mL) was added 2-nitrobenzoyl chloride (1.28 g, 6.9 mmol) at rt. After the mixture was stirred at rt overnight, it was quenched with water (5 mL) and extracted with DCM (10 mL×2). The combined organic layers were dried over anhydrous Na₂SO₄, filtered and concentrated under reduced pressure. The residue was purified by silica gel column chromatography (petroleum ether/ethyl acetate=4:1) to provide the desired product (1.4 g, 85% yield) as a white solid. MS (ESI) m/z=289.1 [M+H]⁺.

### Step 4. Synthesis of 2-amino-N-(1-isopropyl-5-methyl-1H-pyrazol-3-yl)benzamide

To a stirred solution of N-(1-isopropyl-1H-pyrazol-3-yl)-2-nitrobenzamide (1.4 g, 4.86 mmol) in MeOH (50 mL) was added Pd/C (280 mg) under N₂. The suspension was degassed under vacuum and purged with hydrogen several times. After stirring at rt overnight under hydrogen balloon, the mixture was filtered through a pad of Celite and the filter cake was washed with MeOH. The filtrate was concentrated under reduced pressure. The residue was purified by silica gel column chromatography (DCM/MeOH=20:1) to provide the desired product (1.02 g, crude) as a white solid. MS (ESI) m/z=259.2 [M+H]⁺.

### Step 5. Synthesis of tert-butyl (2-(2-(3-((2-((1-isopropyl-5-methyl-1H-pyrazol-3-yl)carbamoyl)phenyl)amino)-3-oxopropoxy)ethoxy)ethyl)carbamate

To a solution of 2,2-dimethyl-4-oxo-3,8,11-trioxa-5-azatetradecan-14-oic acid (318 mg, 1.15 mmol) in DMF (5 mL) were added DIPEA (368 mg, 2.85 mmol), HATU (437 mg, 1.15 mmol) and 2-amino-N-(1-isopropyl-1H-pyrazol-3-yl)benzamide (250 mg, 0.95 mmol) at rt. After stirring at rt overnight, the reaction mixture was quenched with water (20 mL) and extracted with ethyl acetate (15 mL×3). The combined organic layers were washed with brine, dried over anhydrous Na₂SO₄, filtered and concentrated under reduced pressure. The residue was purified by silica gel column chromatography (petroleum ether/ethyl acetate=1:1) to provide the desired product (281 mg, 57% yield) as a white solid. ¹HNMR (400 MHz, DMSO-d₆) δ 10.91 (s, 1H), 10.84 (s, 1H), 8.30 (d, J=8.0 Hz, 1H), 7.83 (d, J=6.8 Hz, 1H), 7.50-7.46 (m, 1H), 7.15-7.11 (m, 1H), 6.71-6.69 (m, 1H), 6.41 (s, 1H), 4.50-4.44 (m, 1H), 3.69 (t, J=6.0 Hz, 2H), 3.53-3.46 (m, 4H), 3.31-3.24 (m, 2H), 3.04-2.99 (m, 2H), 2.56 (t, J=6.0 Hz, 2H), 2.27 (s, 3H), 1.36 (s, 9H), 1.35 (d, J=6.4 Hz, 6H). MS (ESI) m/z=518.4 [M+H]⁺.

### Step 6. Synthesis of 2-(3-(2-(2-aminoethoxy)ethoxy)propanamido)-N-(1-isopropyl-5-methyl-1H-pyrazol-3-yl)benzamide

To a solution of tert-butyl (2-(2-(3-((2-((1-isopropyl-5-methyl-1H-pyrazol-3-yl)carbamoyl)phenyl)amino)-3-oxopropoxy)ethoxy)ethyl)carbamate (10 mg, 0.019 mmol) in DCM (2 mL) was added TFA (1 mL) at 0° C. After stirring at rt for 1 h, the reaction mixture was concentrated under reduced pressure to afford the desired product (8 mg, 80% yield) as TFA salt. MS (ESI) m/z=418.3 [M+H]⁺.

### Example 107. 2-(3-(2-(2-Aminoethoxy)ethoxy)propanamido)-N-(5-methyl-4-(trifluoromethyl)thiazol-2-yl)benzamide (BL1-72)

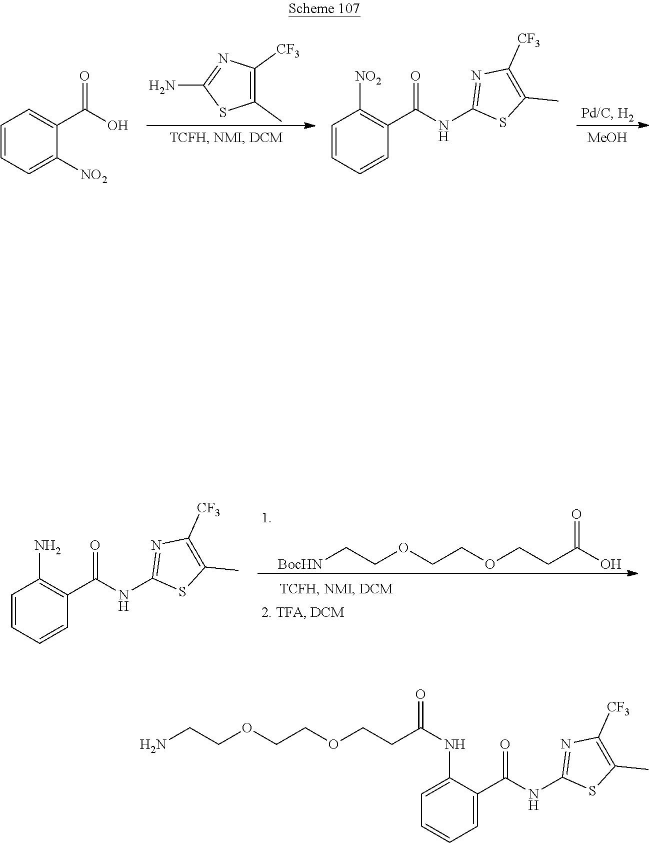

BL1-72 was synthesized following the standard procedures for preparing BL1-55 (9 mg, 22% yield over 4 steps) as TFA salt. MS (ESI) m/z=461.2 [M+H]⁺.

### Example 108. N-(4,5-dimethylthiazol-2-yl)-3-((10-hydroxydecyl)amino)-2-methylbenzamide (BL1-73)

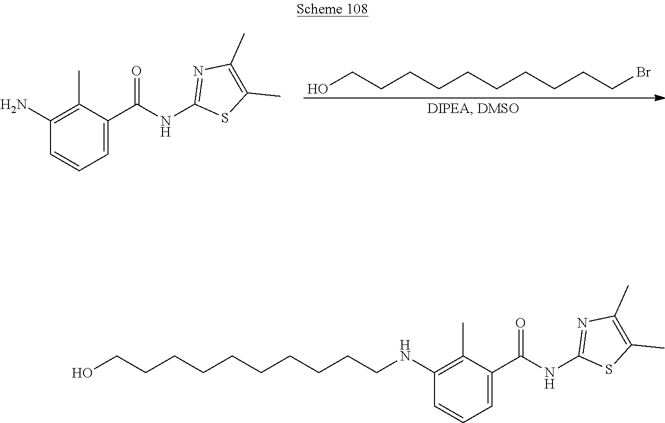

A mixture of 3-amino-N-(4,5-dimethylthiazol-2-yl)-2-methylbenzamide (250 mg, 1.0 mmol), 10-bromodecan-1-ol (300 mg, 1.26 mmol) and DIPEA (387 mg, 3.0 mmol) in DMSO (8.0 mL) was stirred at 80° C. overnight. After cooling down to rt, the reaction mixture was diluted with water (20 mL) and extracted with EtOAc (15 mL×3). The combined organic layers were washed with brine, dried over Na₂SO₄, filtered and concentrated under reduced pressure. The residue was purified by silica gel flash chromatography (petroleum ether/ethyl acetate=1:1) to provide the desired product (90 mg, 25% yield) as a white solid. ¹HNMR (400 MHz, DMSO-d₆) δ 11.96 (s, 1H), 7.01 (t, J=7.8 Hz, 1H), 6.58-6.55 (m, 2H), 4.91 (s, 1H), 3.33-3.23 (m, 2H), 3.02 (t, J=7.0 Hz, 2H), 2.19 (s, 3H), 2.09 (s, 3H), 1.98 (s, 3H), 1.54-1.49 (m, 2H), 1.34-1.16 (m, 14H). MS (ESI) m/z=418.3 [M+H]⁺.

### Example 109. 2-(3-(2-(2-Aminoethoxy)ethoxy)propanamido)-N-(5-cyclopropyl-1-methyl-1H-pyrazol-3-yl)benzamide (BL1-74)

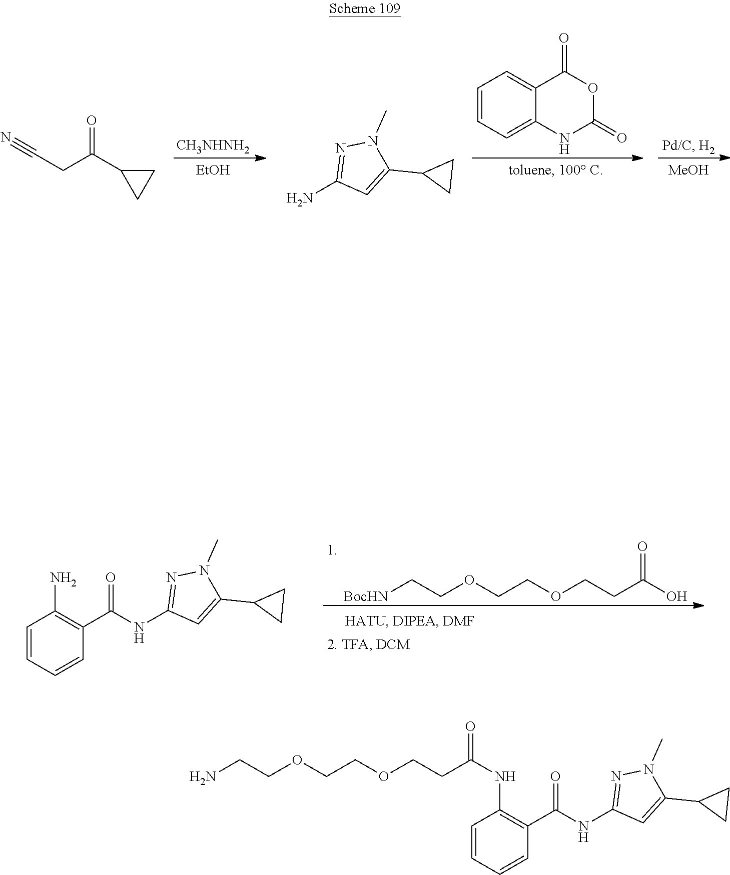

### Step 1. Synthesis of 5-cyclopropyl-1-methyl-1H-pyrazol-3-amine

To a solution of 3-cyclopropyl-3-oxopropanenitrile (1 g, 9.17 mmol) in ethanol (15 mL) was added methyl hydrazine (844 mg, 18.3 mmol) at rt. The reaction mixture was refluxed for 12 h. After cooling down to rt, the reaction was quenched with cold water (10 mL) and extracted with ethyl acetate (15 mL×3). The combined organic layers were washed with brine, dried over Na₂SO₄, filtered and concentrated under reduced pressure. The residue was purified by silica gel column chromatography (DCM/MeOH=20:1) to provide the desired product (1.16 g, 92% yield) as a white solid. MS (ESI) m/z=138.0 [M+H]⁺.

### Step 2. Synthesis of 2-amino-N-(5-cyclopropyl-1-methyl-1H-pyrazol-3-yl)benzamide

The title compound was synthesized following the standard procedure for preparing BL1-53 (693 mg, 32% yield) as a white solid. MS (ESI) m/z=257.1 [M+H]⁺.

### Step 3. Synthesis of tert-butyl (2-(2-(3-((2-((5-cyclopropyl-1-methyl-1H-pyrazol-3-yl)carbamoyl)phenyl)amino)-3-oxopropoxy)ethoxy)ethyl)carbamate

The title compound was synthesized following the standard procedure for preparing BL1-53 (104 mg, 35% yield) as a white solid. ¹HNMR (400 MHz, DMSO-d₆) δ 10.58 (s, 1H), 10.37 (s, 1H), 8.19 (d, J=8.0 Hz, 1H), 7.82 (d, J=8.0 Hz, 1H), 7.57-7.53 (m, 1H), 7.23 (t, J=7.2 Hz, 1H), 6.73-6.72 (m, 1H), 5.97 (s, 1H), 3.68 (t, J=5.6 Hz, 2H), 3.61 (s, 3H), 3.50-3.45 (m, 4H), 3.38-3.35 (m, 2H), 3.04-3.00 (m, 2H), 2.58-2.55 (m, 2H), 1.84-1.78 (m, 1H), 1.36 (s, 9H), 0.85-0.80 (m, 2H), 0.63-0.59 (m, 2H). MS (ESI) m/z=516.1 [M+H]⁺.

### Step 4. Synthesis of 2-(3-(2-(2-aminoethoxy)ethoxy)propanamido)-N-(5-cyclopropyl-1-methyl-1H-pyrazol-3-yl)benzamide

The title compound was synthesized following the standard procedure for preparing BL1-53 (8.0 mg, 80% yield) as TFA salt. MS (ESI) m/z=416.3 [M+H]⁺.

### Example 110. 2-(3-(2-(2-Aminoethoxy)ethoxy)propanamido)-N-(5-methyl-4-(1-methylpiperidin-4-yl)thiazol-2-yl)benzamide (BL1-75)

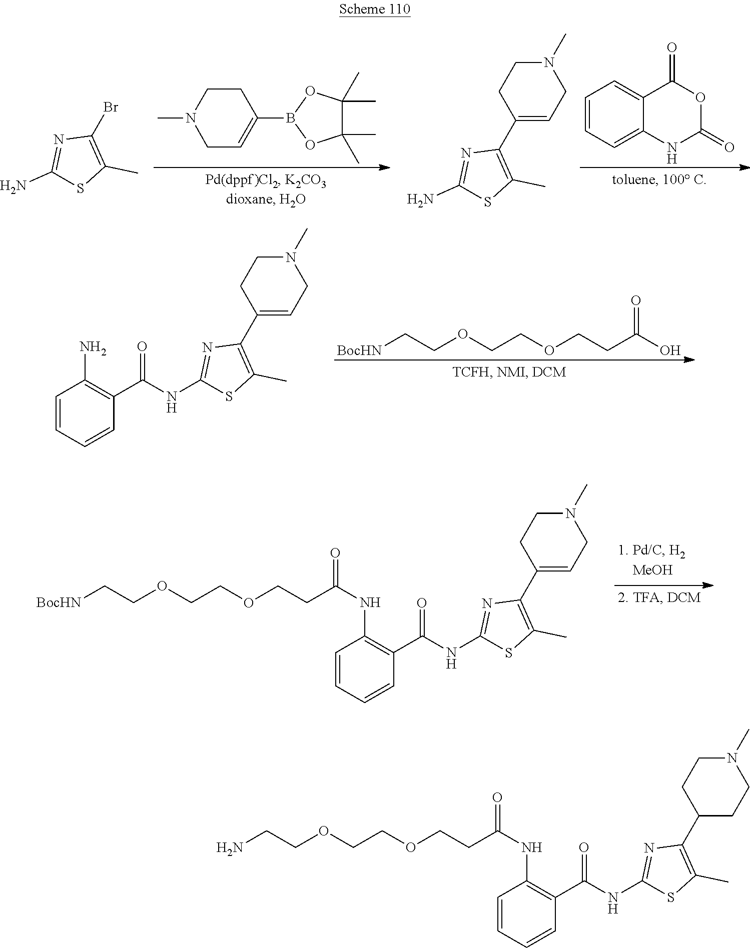

### Step 1. Synthesis of 5-methyl-4-(1-methyl-1,2,3,6-tetrahydropyridin-4-yl)thiazol-2-amine

The title compound was synthesized following the standard procedure for preparing BL1-57 (mg, % yield) as a white solid. MS (ESI) m/z=210.1 [M+H]⁺.

### Step 2. Synthesis of 2-amino-N-(5-methyl-4-(1-methyl-1,2,3,6-tetrahydropyridin-4-yl)thiazol-2-yl)benzamide

The title compound was synthesized following the standard procedure for preparing BL1-57 (mg, % yield) as a white solid. MS (ESI) m/z=329.2 [M+H]⁺.

### Step 3. Synthesis of tert-butyl (2-(2-(3-((2-((5-methyl-4-(1-methyl-1,2,3,6-tetrahydropyridin-4-yl)thiazol-2-yl)carbamoyl)phenyl)amino)-3-oxopropoxy)ethoxy)ethyl)carbamate

The title compound was synthesized following the standard procedure for preparing BL1-55 (mg, % yield) as a white solid. MS (ESI) m/z=588.3 [M+H]⁺.

### Step 4. Synthesis of tert-butyl (2-(2-(3-((2-((5-methyl-4-(1-methylpiperidin-4-yl)thiazol-2-yl)carbamoyl)phenyl)amino)-3-oxopropoxy)ethoxy)ethyl)carbamate

To a solution of tert-butyl (2-(2-(3-((2-((5-methyl-4-(1-methyl-1,2,3,6-tetrahydropyridin-4-yl)thiazol-2-yl)carbamoyl)phenyl)amino)-3-oxopropoxy)ethoxy)ethyl)carbamate (18 mg, 0.03 mmol) in MeOH (10 mL) was added 10% Pd/C (10 mg). The reaction mixture was stirred at rt for 1 h under hydrogen balloon. Then the reaction was filtered through Celite and the filtrate was concentrated under reduced pressure. The residue was purified by reverse-phase chromatography to provide the desired product (15 mg, 83% yield) as a white solid. MS (ESI) m/z: 590.3 [M+H]⁺.

### Step 5. Synthesis of 2-(3-(2-(2-aminoethoxy)ethoxy)propanamido)-N-(5-methyl-4-(1-methylpiperidin-4-yl)thiazol-2-yl)benzamide

The title compound was synthesized following the standard procedure for preparing BL1-55 (10.0 mg, 80% yield) as TFA salt. MS (ESI) m/z=490.3 [M+H]⁺.

### Example 111. 2-(3-(2-(2-aminoethoxy)ethoxy)propanamido)-N-(5-fluoropyridin-2-yl)benzamide (BL1-76)

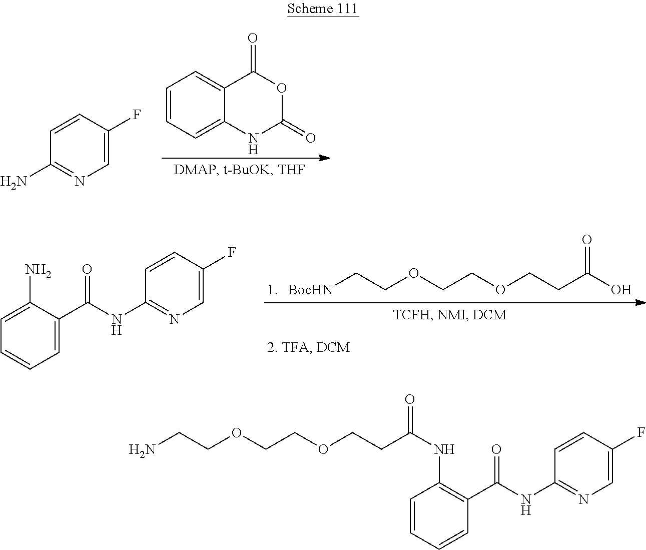

### Step 1. Synthesis of 2-amino-N-(5-fluoropyridin-2-yl)benzamide

To a solution of 1H-benzo[d][1,3]oxazine-2,4-dione (815.5 mg, 5 mmol) in THF (20 ml) were added DMAP (61 mg, 0.5 mmol), t-BuOK (1.234 g, 11 mmol) and 5-fluoropyridin-2-amine (616.6 mg, 5.5 mmol) at rt. After the reaction mixture was stirred at rt overnight, it was quenched with H₂O (20 mL) and extracted with ethyl acetate (15 mL×3). The combined organic layers were washed with brine, dried over sodium sulfate, filtrated and concentrated under reduced pressure. The residue was purified by silica gel column chromatography (petroleum ether/ethyl acetate=10:1) to provide the desired product (330 mg, 28% yield) as a pink solid. ¹HNMR (400 MHz, CDCl₃) δ 8.64 (brs, 1H), 8.34-8.31 (m, 1H), 8.11 (d, J=2.8 Hz, 1H), 7.52-7.44 (m, 2H), 7.28-7.24 (m, 1H), 6.73-6.68 (m, 2H), 5.60 (brs, 2H). ¹⁹FNMR (400 MHz, CDCl₃) δ 132.64. MS (ESI) m/z=232.1 [M+H]⁺.

The remaining steps were performed according to the procedures for preparing BL1-55 to provide the desired product (25 mg, 68% yield over 2 steps) as TFA salt. MS (ESI) m/z=391.2 [M+H]⁺.

### Example 112. 2-(3-(2-(2-Aminoethoxy)ethoxy)propanamido)-N-(5-chloropyridin-2-yl)benzamide (BL1-77)

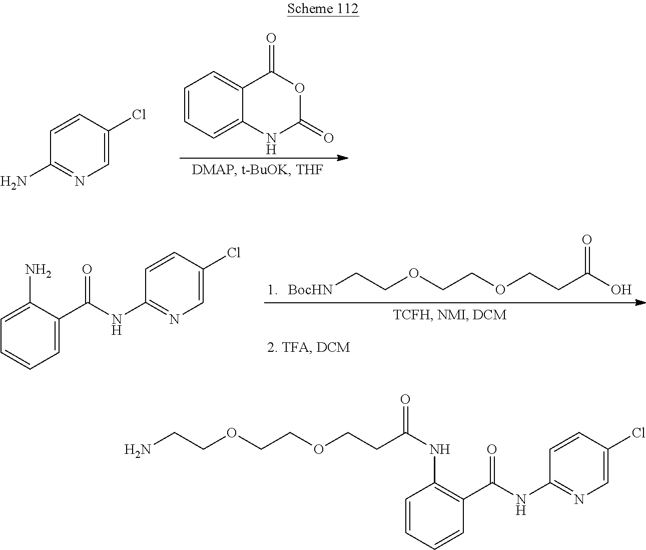 [SMILES: Nc1ccc(Cl)cn1 ; O=c1[nH]c2ccccc2c(=O)o1 ; Nc1ccccc1C(=O)Nc1ccc(Cl)cn1 ; CC(C)(C)OC(=O)NCCOCCOCCC(=O)O ; NCCOCCOCCC(=O)Nc1ccccc1C(=O)Nc1ccc(Cl)cn1]

### Step 1. Synthesis of 2-amino-N-(5-chloropyridin-2-yl)benzamide

The title compound was synthesized following the standard procedure for preparing BL1-76 (490 mg, 41% yield) as a brown solid. ¹HNMR (400 MHz, CDCl₃) δ 8.62 (brs, 1H), 8.30 (d, J=8.8 Hz, 1H), 8.21 (d, J=2.4 Hz, 1H), 7.69 (dd, J=8.8, 2.4 Hz, 1H), 7.52 (dd, J=8.0, 1.2 Hz, 1H), 7.29-7.25 (m, 1H), 6.73-6.68 (m, 2H), 5.62 (brs, 2H). MS (ESI) m/z=248.1 [M+H]⁺.

The remaining steps were performed according to the procedures for preparing BL1-55 to provide the desired product (30 mg, 62% yield over 2 steps) as TFA salt. MS (ESI) m/z=407.2 [M+H]⁺

### Example 113. 2-(3-(2-(2-Aminoethoxy)ethoxy)propanamido)-N-(5-cyanopyridin-2-yl)benzamide (BL1-78)

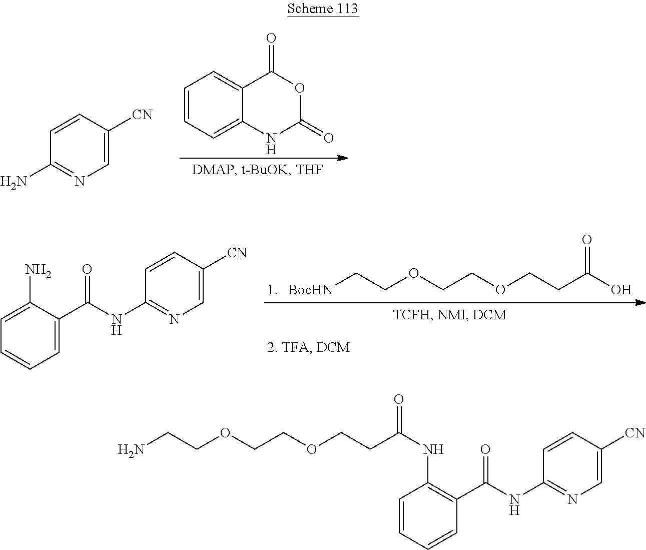 [SMILES: N#Cc1ccc(N)nc1 ; O=c1[nH]c2ccccc2c(=O)o1 ; N#Cc1ccc(NC(=O)c2ccccc2N)nc1 ; CC(C)(C)OC(=O)NCCOCCOCCC(=O)O ; N#Cc1ccc(NC(=O)c2ccccc2NC(=O)CCOCCOCCN)nc1]

### Step 1. Synthesis of 2-amino-N-(5-cyanopyridin-2-yl)benzamide

The title compound was synthesized following the standard procedure for preparing BL1-76 (135 mg, 11% yield) as a yellow solid. ¹HNMR (400 MHz, CDCl₃) δ 8.78 (brs, 2H), 8.55-8.54 (m, 1H), 8.47-8.44 (m, 1H), 7.97-7.95 (m, 1H), 7.53-7.51 (m, 1H), 7.33-7.26 (m, 2H), 5.68 (brs, 2H). MS (ESI) m/z=239.1 [M+H]⁺.

The remaining steps were performed according to the procedures for preparing BL1-55 to provide the desired product (20 mg, 60% yield over 2 steps) as TFA salt. MS (ESI) m/z=398.2 [M+H]⁺.

### Example 114. 2-(3-(2-(2-Aminoethoxy)ethoxy)propanamido)-N-(5-(trifluoromethyl)pyridin-2-yl)benzamide (BL1-79)

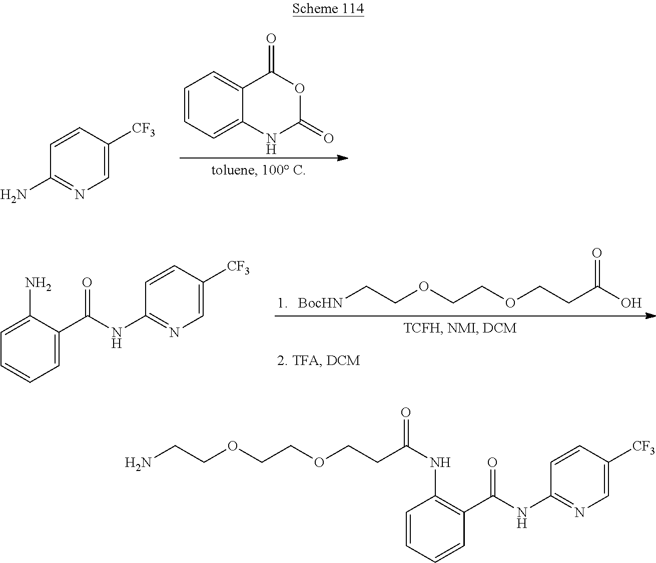 [SMILES: Nc1ccc(C(F)(F)F)cn1 ; O=c1[nH]c2ccccc2c(=O)o1 ; Nc1ccccc1C(=O)Nc1ccc(C(F)(F)F)cn1 ; CC(C)(C)OC(=O)NCCOCCOCCC(=O)O ; NCCOCCOCCC(=O)Nc1ccccc1C(=O)Nc1ccc(C(F)(F)F)cn1]

### Step 1. Synthesis of 2-amino-N-(5-(trifluoromethyl)pyridin-2-yl)benzamide

The title compound was synthesized following the standard procedure for preparing BL1-53 (208 mg, 40% yield) as a white solid. ¹HNMR (400 MHz, DMSO-d₆) δ 10.84 (s, 1H), 8.75 (s, 1H), 8.30 (d, J=8.8 Hz, 1H), 8.22-8.19 (m, 1H), 7.75 (dd, J=8.0, 1.2 Hz, 1H), 7.25-7.21 (m, 1H), 6.77 (d, J=8.0 Hz, 1H), 6.58-6.48 (m, 1H), 6.49 (brs, 2H). MS (ESI) m/z=282.1 [M+H]⁺.

The remaining steps were performed according to the procedures for preparing BL1-55 to provide the desired product (40 mg, 64% yield over 2 steps) as TFA salt. MS (ESI) m/z=441.2 [M+H]⁺.

### Example 115. 2-(3-(2-(2-Aminoethoxy)ethoxy)propanamido)-N-(6-methoxypyridazin-3-yl)benzamide (BL1-80)

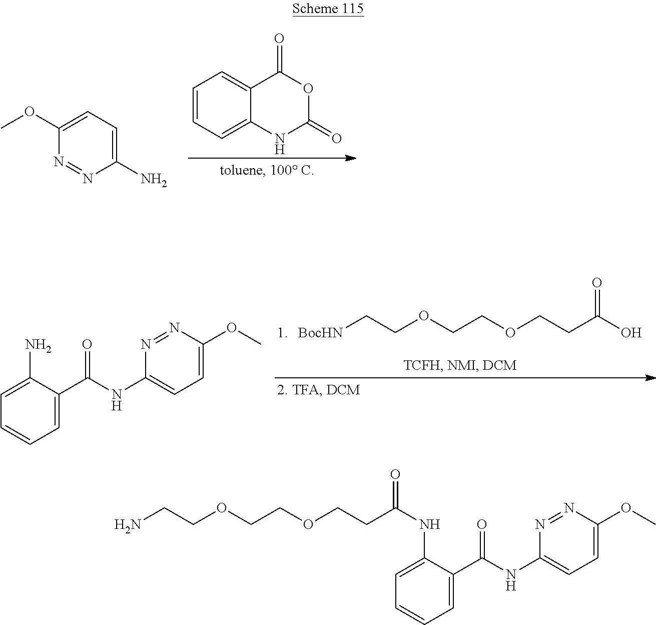

### Step 1. Synthesis of 2-amino-N-(6-methoxypyridazin-3-yl)benzamide

The title compound was synthesized following the standard procedure for preparing BL1-76 (260 mg, 36% yield) as a yellow solid. ¹HNMR (400 MHz, CDCl₃) δ 9.00 (brs, 1H), 8.44 (d, J=9.6 Hz, 1H), 7.61 (dd, J=8.4, 1.2 Hz, 1H), 7.30-7.26 (m, 1H), 7.04 (d, J=9.6 Hz, 1H), 6.74-6.71 (m, 2H), 5.64 (brs, 2H), 7.11 (s, 3H). MS (ESI) m/z=245.1 [M+H]⁺.

The remaining steps were performed according to the procedures for preparing BL1-55 to provide the desired product (50 mg, 75% yield over 2 steps) as TFA salt. MS (ESI) m/z=404.2 [M+H]⁺.

### Example 116. 2-(8-hydroxyoctanamido)-N-(5-methylpyridin-2-yl)benzamide (BL1-178)

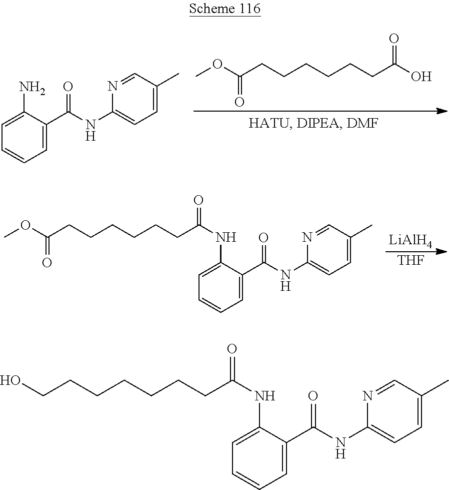

### Step 1. Synthesis of methyl 8-((2-((5-methylpyridin-2-yl)carbamoyl)phenyl)amino)-8-oxooctanoate

To a solution of 2-amino-N-(5-methylpyridin-2-yl)benzamide (94 mg, 0.50 mmol) in DMF (5 mL) were added 8-methoxy-8-oxooctanoic acid (120 mg, 0.53 mmol), HATU (250 mg, 0.66 mmol) and DIPEA (200 mg, 1.5 mmol). After the reaction mixture was stirred at rt overnight, it was quenched with H₂O (20 mL) and extracted with EtOAc (15 mL×3). The combined organic layers were washed with brine, dried over Na₂SO₄, filtered and concentrated under reduced pressure. The residue was purified by silica gel column chromatography (petroleum ether/ethyl acetate=1:1) to provide the desired product (120 mg, 61% yield) as a colorless oil. MS (ESI) m/z=398.2 [M+H]⁺.

### Step 2. Synthesis of 2-(8-hydroxyoctanamido)-N-(5-methylpyridin-2-yl)benzamide

To a solution of methyl 8-((2-((5-methylpyridin-2-yl)carbamoyl)phenyl)amino)-8-oxooctanoate (300 mg, 0.75 mmol) in THF (5.0 mL) was added a solution of LiAlH4 (1M in THF, 1.0 mL, 1.0 mmol) at ° C. After stirring at 0° C. for 5 min, the reaction was quenched with Na₂SO₄·10H₂O. The mixture was filtered and the filtrate was concentrated under reduced pressure. The residue was purified by silica gel column chromatography (petroleum/ethyl acetate=1:1) to provide the desired product (140 mg, 50% yield) as a colorless oil. ¹HNMR (400 MHz, DMSO-d₆) δ 10.66 (s, 1H), 10.40 (s, 1H), 8.21 (s, 1H), 8.07-8.00 (m, 2H), 7.81-7.78 (m, 1H), 7.67-7.64 (m, 1H), 7.53-7.48 (m, 1H), 7.21-7.16 (m, 1H), 4.31 (t, J=5.2 Hz, 2H), 3.37-3.33 (m, 2H), 2.32-2.28 (m, 5H), 1.58-1.51 (m, 2H), 1.39-1.34 (m, 2H), 1.30-1.20 (m, 6H). MS (ESI) m/z=370.3 [M+H]⁺.

### Example 117. 2-(3-(2-(2-Aminoethoxy)ethoxy)propanamido)-N-(5-cyclopropylpyridin-2-yl)benzamide (BL1-179)

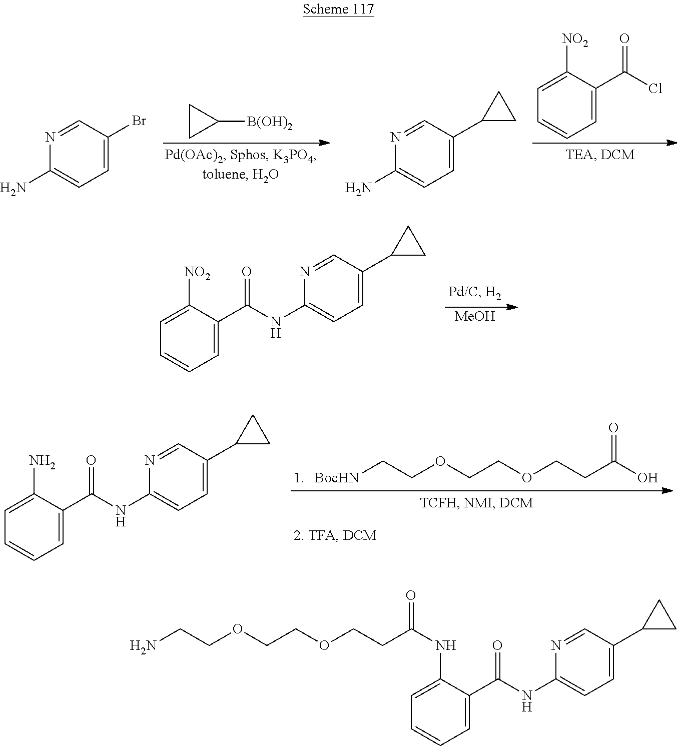

### Step 1. Synthesis of 5-cyclopropylpyridin-2-amine

To a solution of 5-bromopyridin-2-amine (1 g, 5.8 mmol) and cyclopropylboronic acid (749 mg, 8.7 mmol) in toluene (40 mL) and H₂O (4 mL) were added Pd(OAc)₂ (130.5 mg, 0.58 mmol), S-Phos (477 mg, 1.16 mmol) and K₃PO₄ (3.69 g, 17.4 mmol) at rt. The reaction mixture was stirred at 95° C. under nitrogen for 12 h. After cooling down to rt, the reaction mixture was quenched with H₂O (10 mL) and extracted with ethyl acetate (30 mL×3). The combined organic layers were washed with brine, dried over Na₂SO₄, filtered and concentrated under reduced pressure. The residue was purified by silica gel column chromatography (petroleum ether/ethyl acetate=1:1) to provide the desired product (662 mg, 85% yield) as a yellow solid. MS (ESI) m/z=135.2 [M+H]⁺.

### Step 2. Synthesis of N-(5-cyclopropylpyridin-2-yl)-2-nitrobenzamide

The title compound was synthesized following the standard procedure for preparing BL1-64 (750 mg, 71% yield) as a white solid. MS (ESI) m/z=284.1 [M+H]⁺.

### Step 3. Synthesis of 2-amino-N-(5-cyclopropylpyridin-2-yl)benzamide

The title compound was synthesized following the standard procedure for preparing BL1-55 (309 mg, 46% yield) as a whiter solid. ¹HNMR (400 MHz, DMSO-d₆) δ 10.27 (brs, 1H), 8.19 (d, J=2.0 Hz, 1H), 7.96 (d, J=8.4 Hz, 1H), 7.71 (dd, J=8.0, 1.6 Hz, 1H), 7.44 (dd, J=8.8, 2.4 Hz, 1H), 7.21-7.17 (m, 1H), 6.74 (dd, J=8.4, 0.8 Hz, 1H), 6.54 (td, J=8.0, 1.2 Hz, 1H), 6.41 (brs, 2H), 1.97-1.91 (m, 1H), 0.99-0.95 (m, 2H), 0.73-0.69 (m, 2H). MS (ESI) m/z=254.2 [M+H]⁺.

The remaining steps were performed according to the procedures for preparing BL1-55 to provide the desired product (15 mg, 46% yield over 2 steps) as TFA salt. MS (ESI) m/z=413.3 [M+H]⁺.

### Example 118. 2-(3-(2-(2-Aminoethoxy)ethoxy)propanamido)-N-(6-(dimethylamino)pyridazin-3-yl)benzamide (BL1-180)

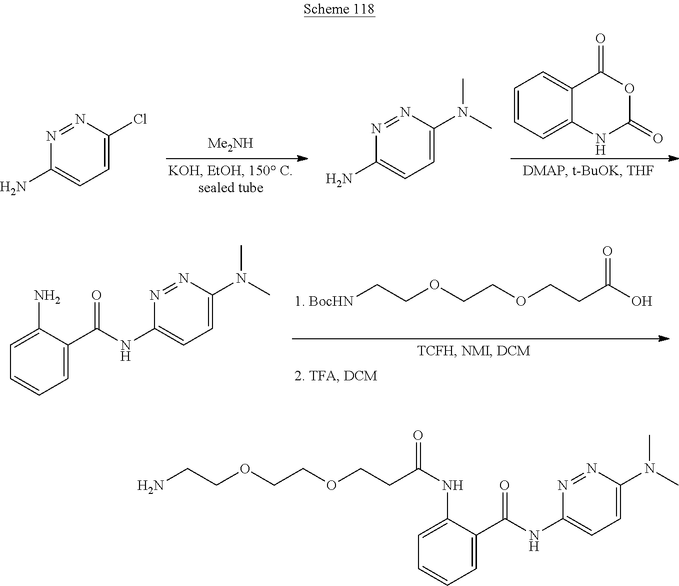

### Step 1. Synthesis of N³,N³-dimethylpyridazine-3,6-diamine

A mixture of 6-chloropyridazin-3-amine (2 g, 15.4 mmol), dimethylamine hydrochloride (6.3 g, 77.0 mmol) and KOH (4.3 g, 77.0 mmol) in ethanol (15 ml) was stirred at 150° C. in the sealed tube for 24 h. After cooling down to rt, the reaction mixture was concentrated under reduced pressure. The residue was diluted with H₂O (15 mL) and extracted with ethyl acetate (20 mL×3). The combined organic layers were washed with brine, dried over sodium sulfate, filtered and concentrated under reduced pressure. The resulting crude product was purified by silica gel column chromatography (DCM/MeOH=30:1 to 10:1) to provide the desired product (950 mg, 45% yield) as a yellow solid. MS (ESI) m/z=139.2 [M+H]⁺.

### Step 2. Synthesis of 2-amino-N-(6-(dimethylamino)pyridazin-3-yl)benzamide

The title compound was synthesized following the standard procedure for preparing BL1-76 (130 mg, 25% yield) as a yellow solid. ¹HNMR (400 MHz, CDCl₃) δ 10.51 (brs, 1H), 7.87 (d, J=9.6 Hz, 1H), 7.75 (dd, J=6.8, 1.2 Hz, 1H), 7.24-7.18 (m, 2H), 6.76 (dd, J=8.4, 0.8 Hz 1H), 6.60-6.56 (m, 1H), 6.44 (brs, 2H), 3.09 (s, 6H). MS (ESI) m/z=258.1 [M+H]⁺.

The remaining steps were performed according to the procedures for preparing BL1-55 to provide the desired product (10 mg, 31% yield over 2 steps) as TFA salt. MS (ESI) m/z=417.3 [M+H]⁺.

### Example 119. 2-(3-(2-(2-Aminoethoxy)ethoxy)propanamido)-N-(2-methylpyrimidin-5-yl)benzamide (BL1-181)

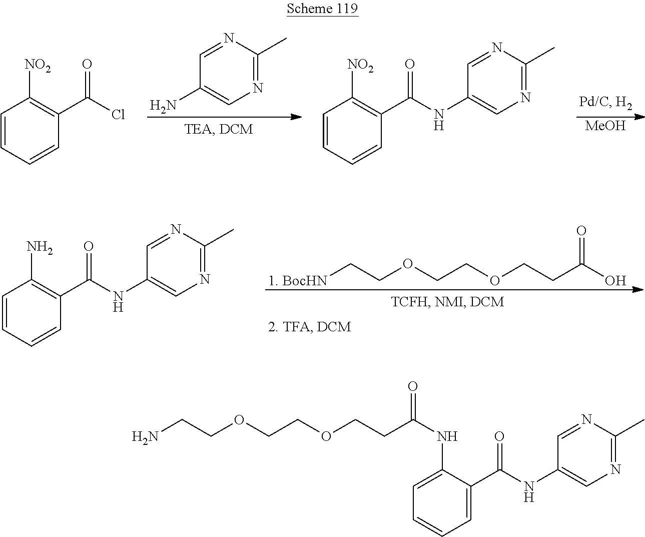

### Step 1. Synthesis of N-(2-methylpyrimidin-5-yl)-2-nitrobenzamide

The title compound was synthesized following the standard procedure for preparing BL1-64 (33 mg, 79% yield) as a white solid. MS (ESI) m/z=259.0 [M+H]⁺.

### Step 2. Synthesis of 2-amino-N-(2-methylpyrimidin-5-yl)benzamide

The title compound was synthesized following the standard procedure for preparing BL1-55 (259 mg, 88% yield) as a white solid. ¹HNMR (400 MHz, DMSO-d₆) δ 10.19 (s, 1H), 8.99 (s, 2H), 7.68 (dd, J=8.0, 1.6 Hz, 1H), 7.25-7.21 (m, 1H), 6.78 (dd, J=8.0, 0.8 Hz, 1H), 6.62-6.58 (m, 1H), 6.46 (s, 2H), 2.56 (s, 3H). MS (ESI) m/z=229.1 [M+H]⁺.

The remaining steps were performed according to the procedures for preparing BL1-55 to provide the desired product (20 mg, 59% yield over 2 steps) as TFA salt. MS (ESI) m/z=388.2 [M+H]⁺.

### Example 120. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methylthiazol-2-yl)benzamide (CPD-051)

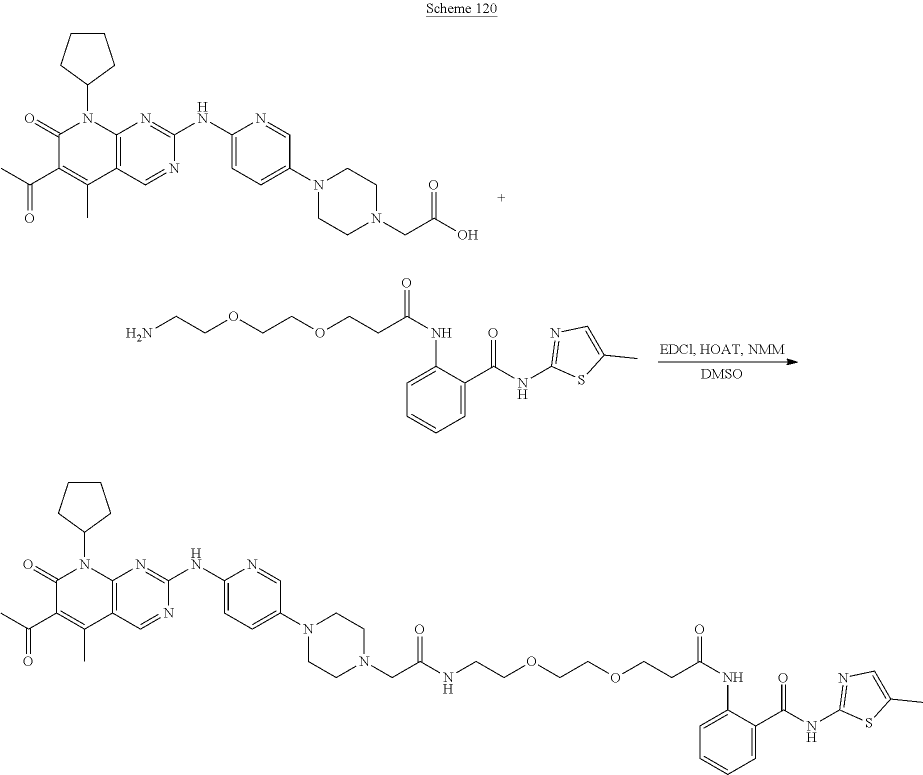 [SMILES: CC(=O)c1c(C)c2cnc(Nc3ccc(N4CCN(CC(=O)O)CC4)cn3)nc2n(C2CCCC2)c1=O ; Cc1cnc(NC(=O)c2ccccc2NC(=O)CCOCCOCCN)s1 ; CC(=O)c1c(C)c2cnc(Nc3ccc(N4CCN(CC(=O)NCCOCCOCCC(=O)Nc5ccccc5C(=O)Nc5ncc(C)s5)CC4)cn3)nc2n(C2CCCC2)c1=O]

CPD-051 was synthesized following the standard procedure for preparing CPD-008 (5.5 mg, 21% yield) as a yellow solid. MS (ESI) m/z=880.4 [M+H]⁺.

### Example 121. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(1,5-dimethyl-1H-pyrazol-3-yl)benzamide (CPD-052)

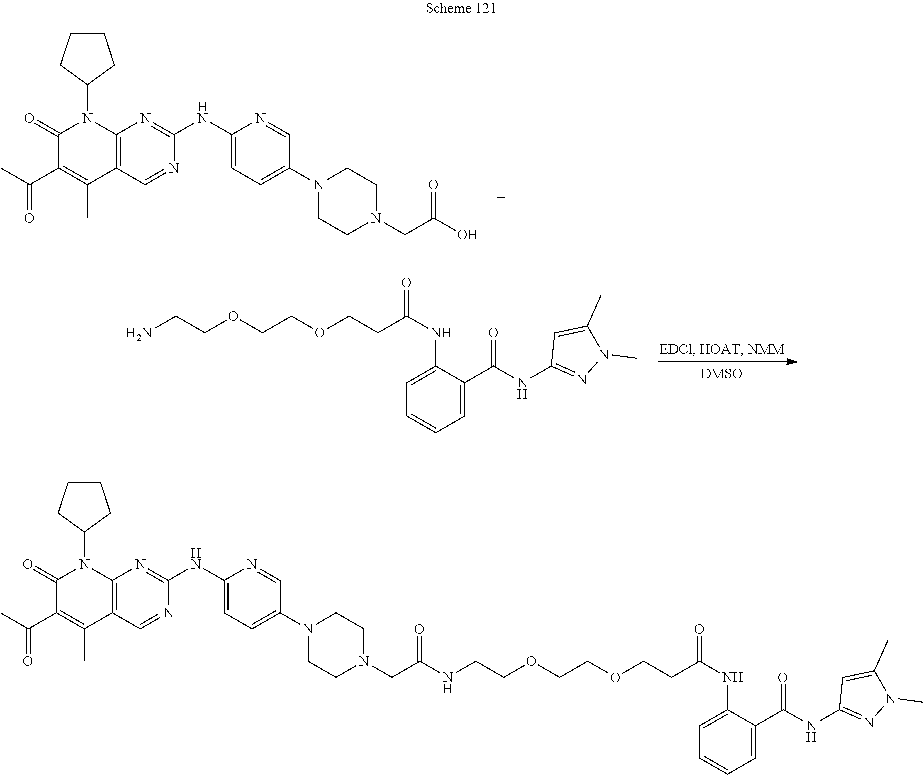 [SMILES: CC(=O)c1c(C)c2cnc(Nc3ccc(N4CCN(CC(=O)O)CC4)cn3)nc2n(C2CCCC2)c1=O ; Cc1cc(NC(=O)c2ccccc2NC(=O)CCOCCOCCN)nn1C ; CC(=O)c1c(C)c2cnc(Nc3ccc(N4CCN(CC(=O)NCCOCCOCCC(=O)Nc5ccccc5C(=O)Nc5cc(C)n(C)n5)CC4)cn3)nc2n(C2CCCC2)c1=O]

CPD-052 was synthesized following the standard procedure for preparing CPD-008 (15.2 mg 34% yield) as a yellow solid. MS (ESI) m/z=877.5 [M+H]⁺.

### Example 122. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(pyridin-2-yl)benzamide (CPD-053)

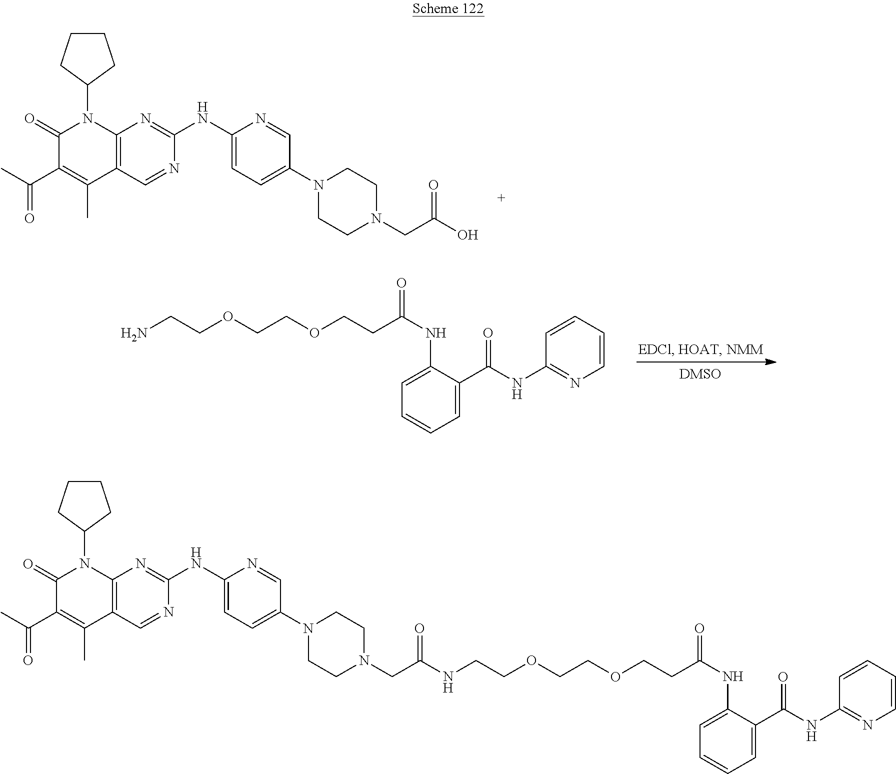 [SMILES: CC(=O)c1c(C)c2cnc(Nc3ccc(N4CCN(CC(=O)O)CC4)cn3)nc2n(C2CCCC2)c1=O ; NCCOCCOCCC(=O)Nc1ccccc1C(=O)Nc1ccccn1 ; CC(=O)c1c(C)c2cnc(Nc3ccc(N4CCN(CC(=O)NCCOCCOCCC(=O)Nc5ccccc5C(=O)Nc5ccccn5)CC4)cn3)nc2n(C2CCCC2)c1=O]

CPD-053 was synthesized following the standard procedure for preparing CPD-008 (1.3 mg, 19% yield) as a yellow solid. MS (ESI) m/z=860.5 [M+H]⁺.

### Example 123. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methylpyrazin-2-yl)benzamide (CPD-054)

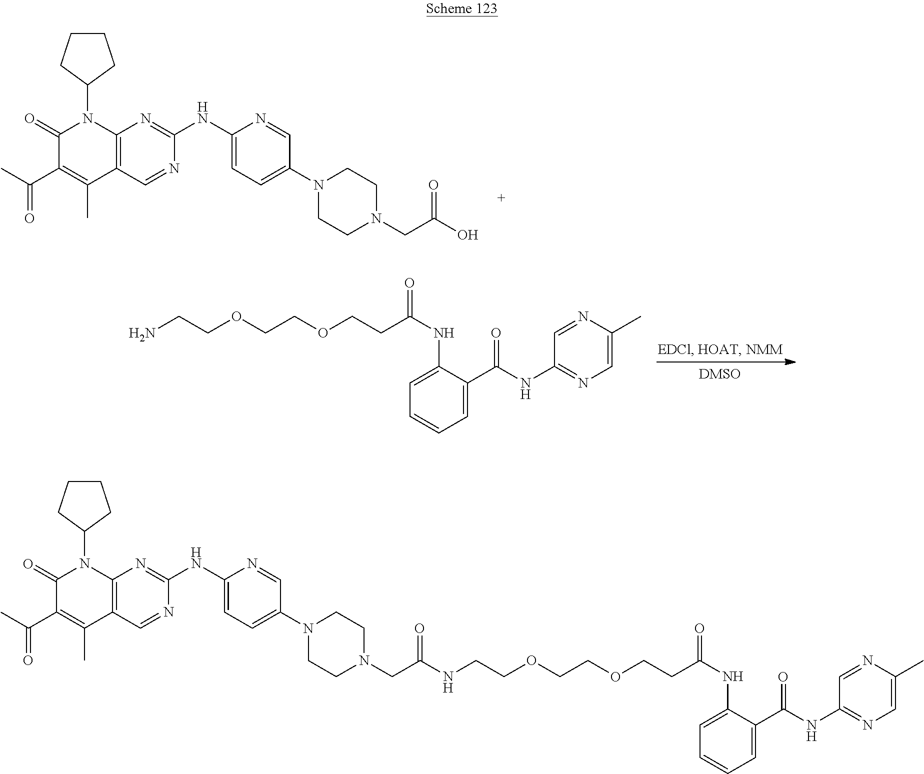 [SMILES: CC(=O)c1c(C)c2cnc(Nc3ccc(N4CCN(CC(=O)O)CC4)cn3)nc2n(C2CCCC2)c1=O ; Cc1cnc(NC(=O)c2ccccc2NC(=O)CCOCCOCCN)cn1 ; CC(=O)c1c(C)c2cnc(Nc3ccc(N4CCN(CC(=O)NCCOCCOCCC(=O)Nc5ccccc5C(=O)Nc5cnc(C)cn5)CC4)cn3)nc2n(C2CCCC2)c1=O]

CPD-054 was synthesized following the standard procedure for preparing CPD-008 (7.0 mg, 50% yield) as a yellow solid. MS (ESI) m/z=875.5 [M+H]⁺.

### Example 124. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methylpyrimidin-2-yl)benzamide (CPD-055)

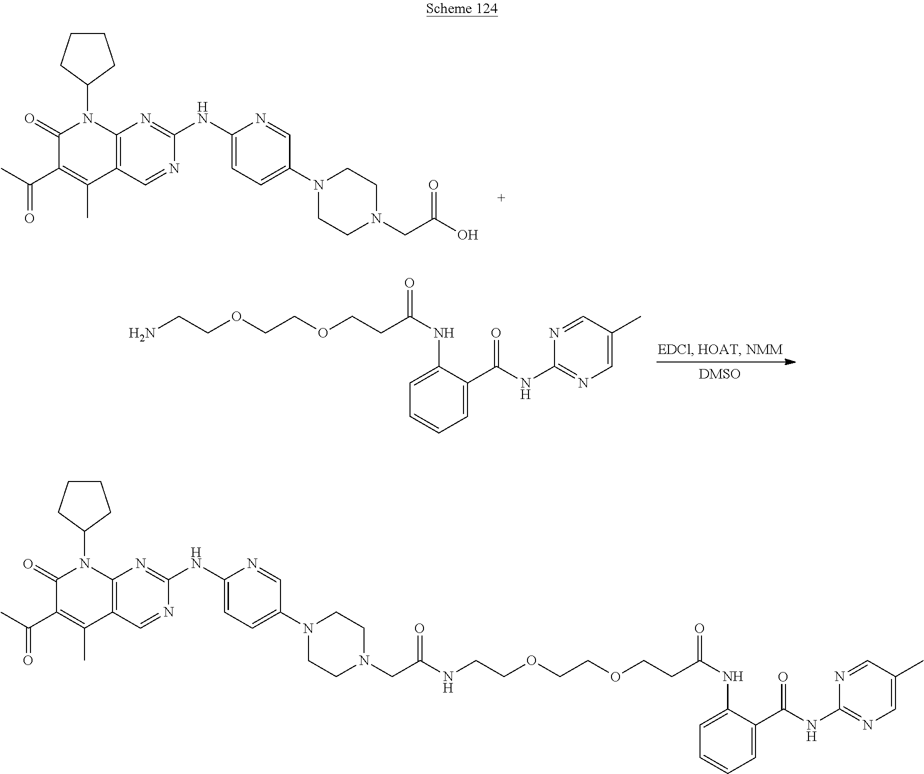 [SMILES: CC(=O)c1c(C)c2cnc(Nc3ccc(N4CCN(CC(=O)O)CC4)cn3)nc2n(C2CCCC2)c1=O ; Cc1cnc(NC(=O)c2ccccc2NC(=O)CCOCCOCCN)nc1 ; CC(=O)c1c(C)c2cnc(Nc3ccc(N4CCN(CC(=O)NCCOCCOCCC(=O)Nc5ccccc5C(=O)Nc5ncc(C)cn5)CC4)cn3)nc2n(C2CCCC2)c1=O]

CPD-055 was synthesized following the standard procedure for preparing CPD-008 (4.0 mg, 24% yield) as a yellow solid. MS (ESI) m/z=875.5 [M+H]⁺.

### Example 125. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(6-methylpyridazin-3-yl)benzamide (CPD-056)

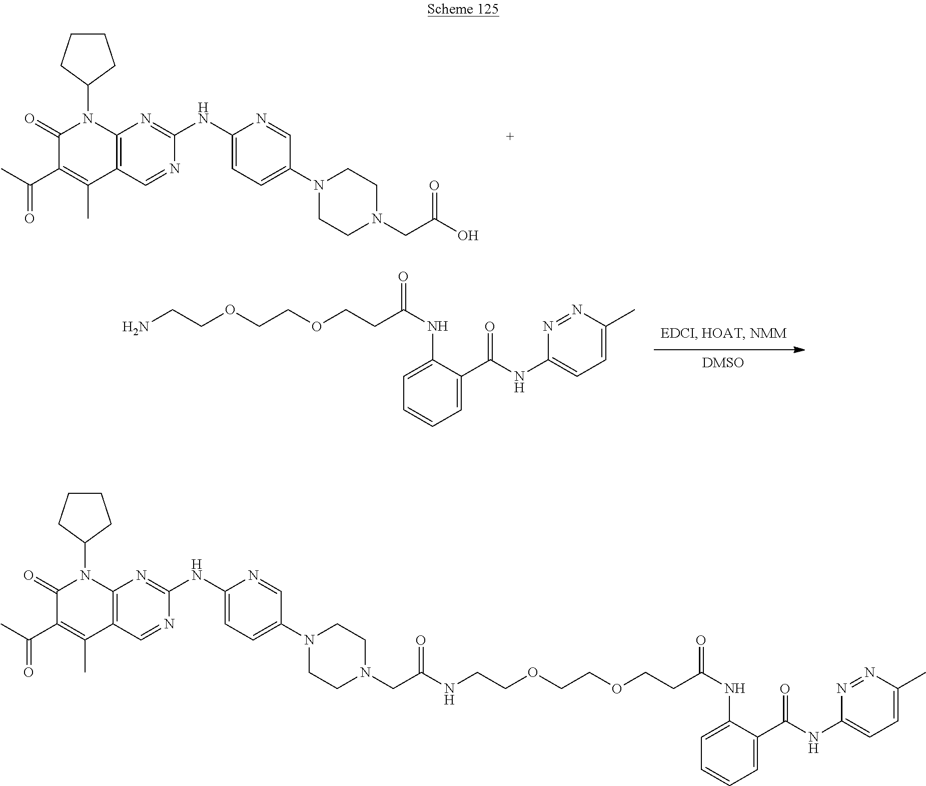

CPD-056 was synthesized following the standard procedure for preparing CPD-008 (9.0 mg, 29% yield) as a yellow solid. MS (ESI) m/z=875.6 [M+H]⁺.

### Example 126. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4-cyclopropyl-5-methylthiazol-2-yl)benzamide (CPD-057)

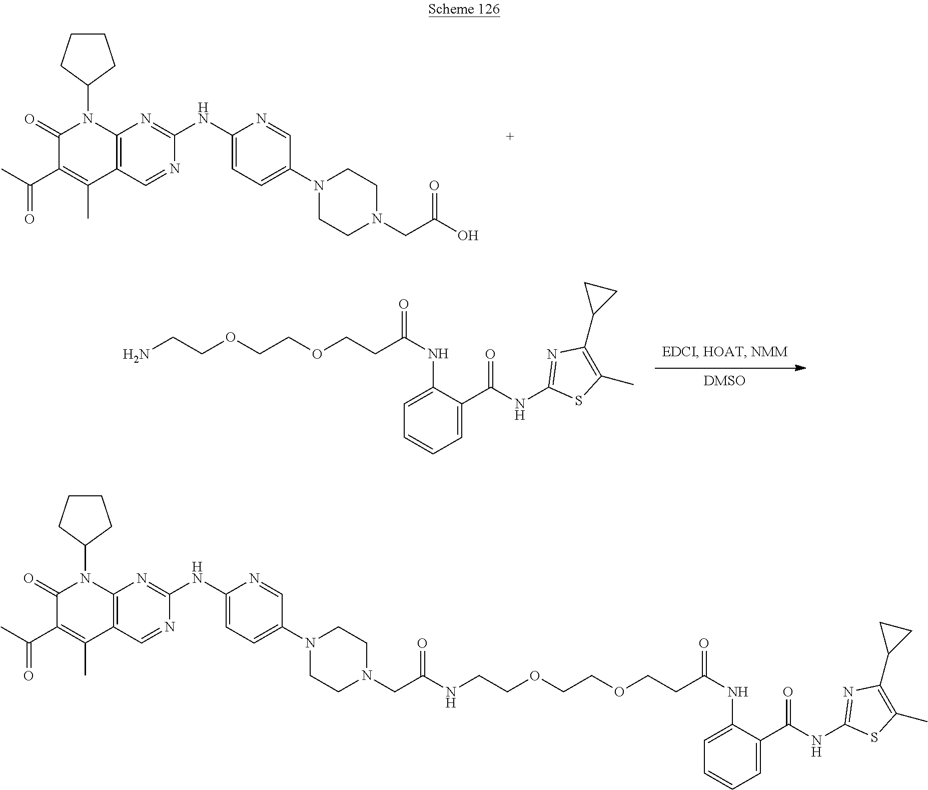

CPD-057 was synthesized following the standard procedure for preparing CPD-008 (2.2 mg, 12% yield) as a yellow solid. MS (ESI) m/z=920.5 [M+H]⁺.

### Example 127. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(3-methyl-1,2,4-thiadiazol-5-yl)benzamide (CPD-058)

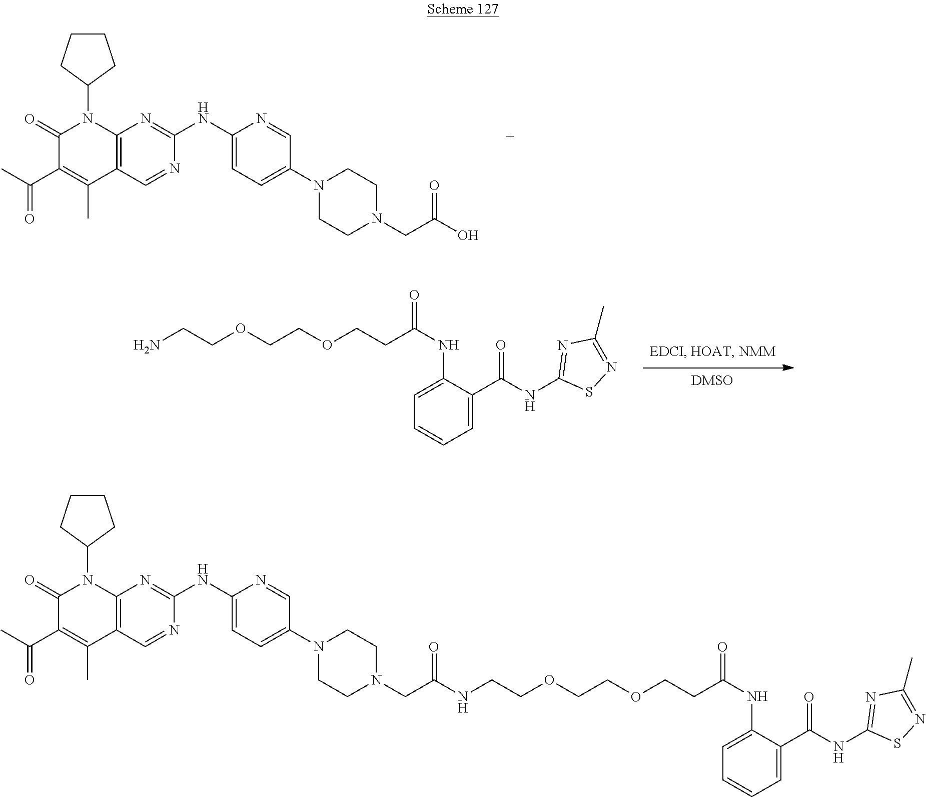

CPD-058 was synthesized following the standard procedure for preparing CPD-008 (6.3 mg, 17% yield) as a yellow solid. MS (ESI) m/z=881.4 [M+H]⁺.

### Example 128. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(3-cyclopropyl-1,2,4-thiadiazol-5-yl)benzamide (CPD-059)

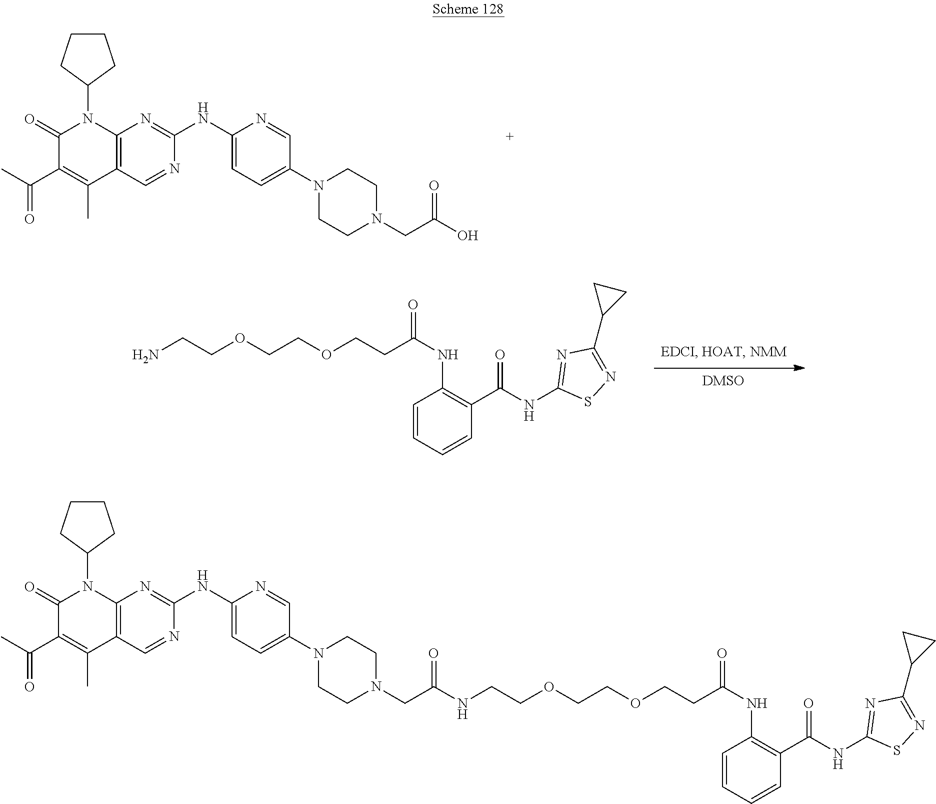

CPD-059 was synthesized following the standard procedure for preparing CPD-008 (6.1 mg, 19% yield) as a yellow solid. MS (ESI) m/z=907.5 [M+H]⁺.

### Example 129. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(6-methylpyridin-3-yl)benzamide (CPD-060)

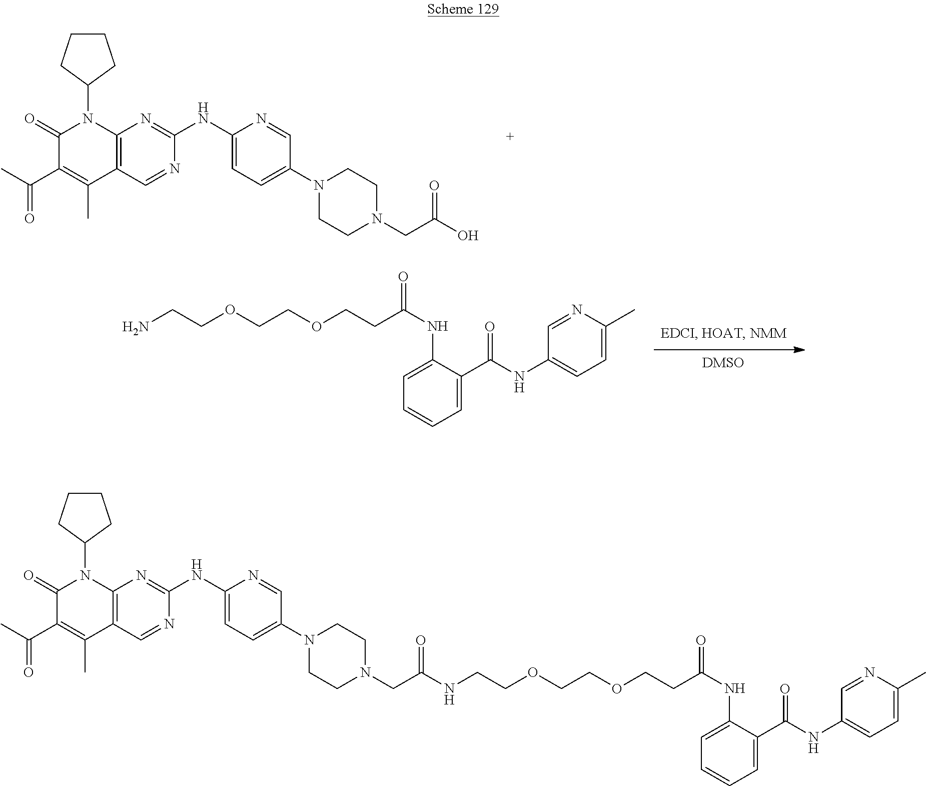

CPD-060 was synthesized following the standard procedure for preparing CPD-008 (24 mg, 36% yield) as a yellow solid. MS (ESI) m/z=874.5 [M+H]⁺.

### Example 130. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methylpyridin-2-yl)benzamide (CPD-061)

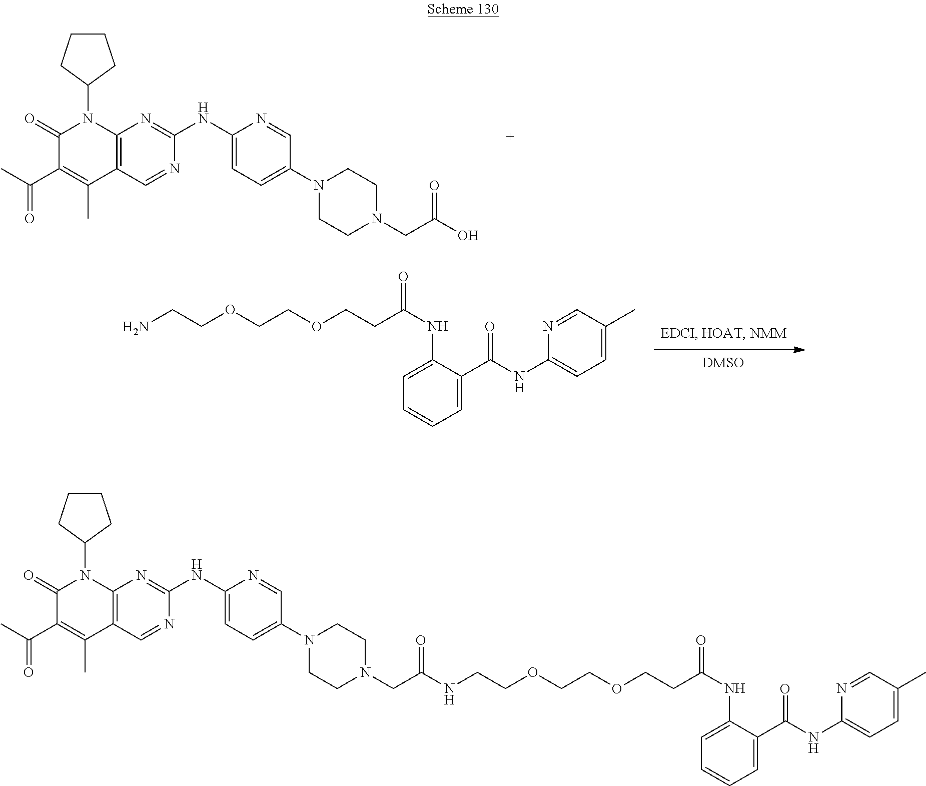

CPD-061 was synthesized following the standard procedure for preparing CPD-008 (7.6 mg, 17% yield) as a yellow solid. MS (ESI) m/z=874.5 [M+H]⁺.

### Example 131. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methyl-4-(tetrahydro-2H-pyran-4-yl)thiazol-2-yl)benzamide (CPD-062)

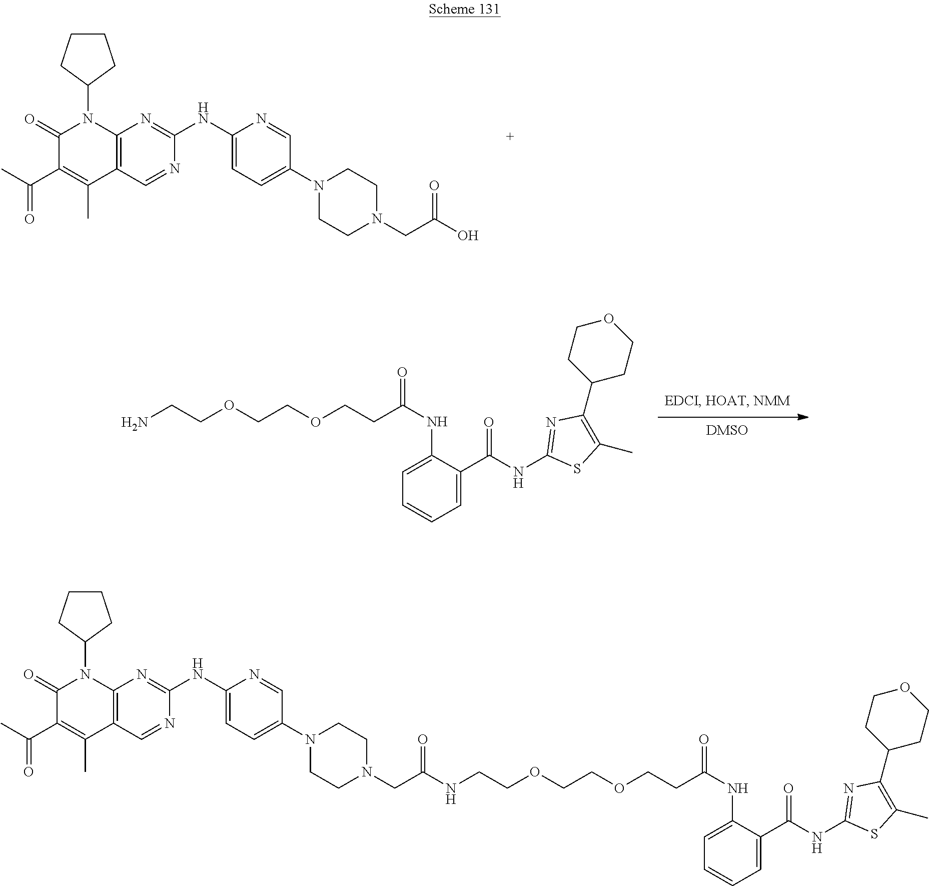

CPD-062 was synthesized following the standard procedure for preparing CPD-008 (5.4 mg, 23% yield) as a yellow solid. MS (ESI) m/z=964.5 [M+H]⁺.

### Example 132. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(1-methyl-1H-imidazol-4-yl)benzamide (CPD-063)

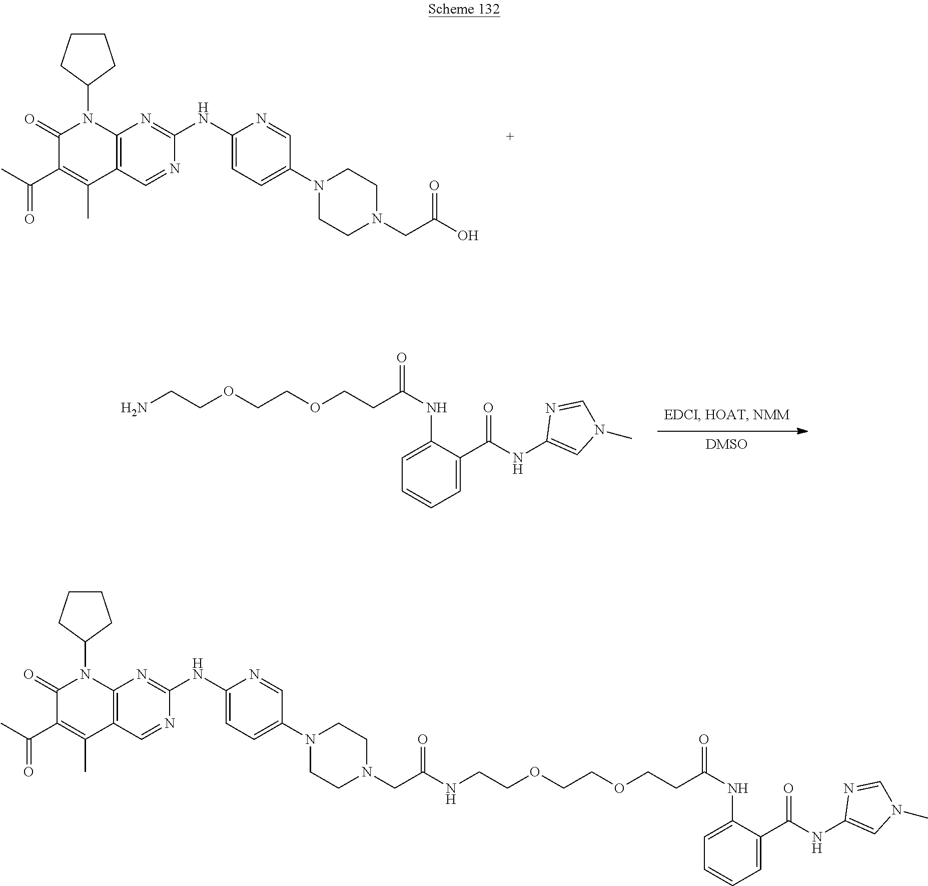

CPD-063 was synthesized following the standard procedure for preparing CPD-008 (6.3 mg, 27% yield) as a yellow solid. MS (ESI) m/z=863.5 [M+H]⁺.

### Example 133. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methyl-1H-imidazol-2-yl)benzamide (CPD-064)

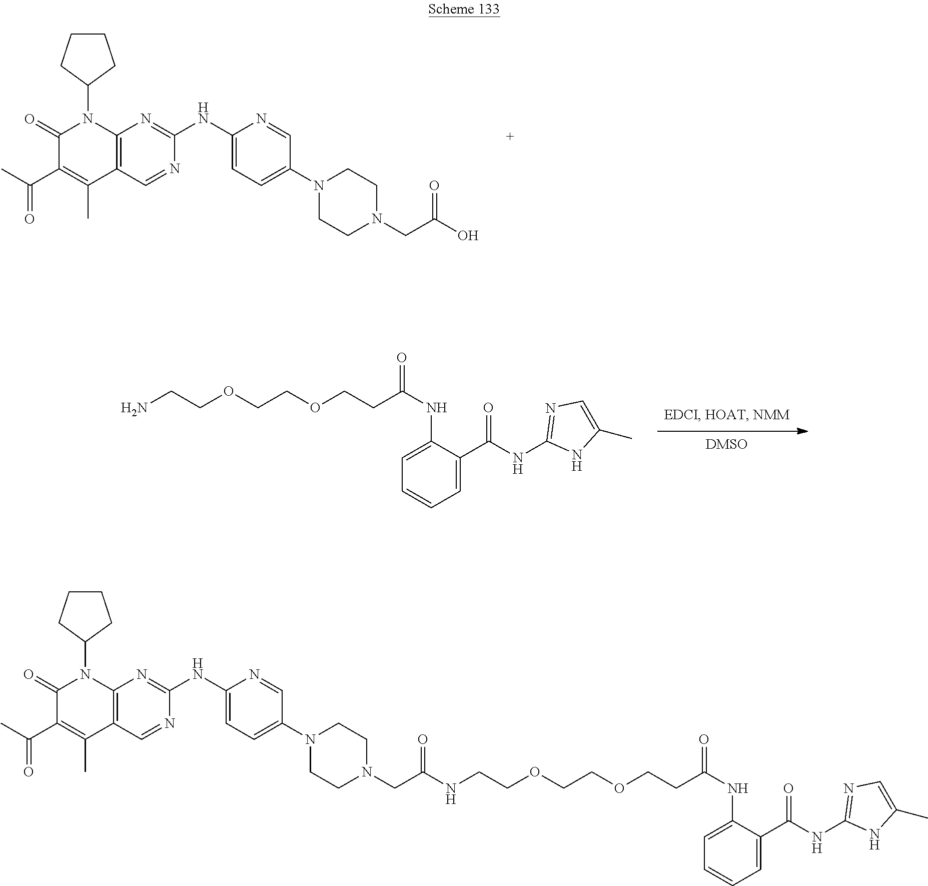

CPD-064 was synthesized following the standard procedure for preparing CPD-008 (7.8 mg, 28% yield) as a yellow solid. MS (ESI) m/z=863.5 [M+H]⁺.

### Example 134. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methylthiophen-2-yl)benzamide (CPD-065)

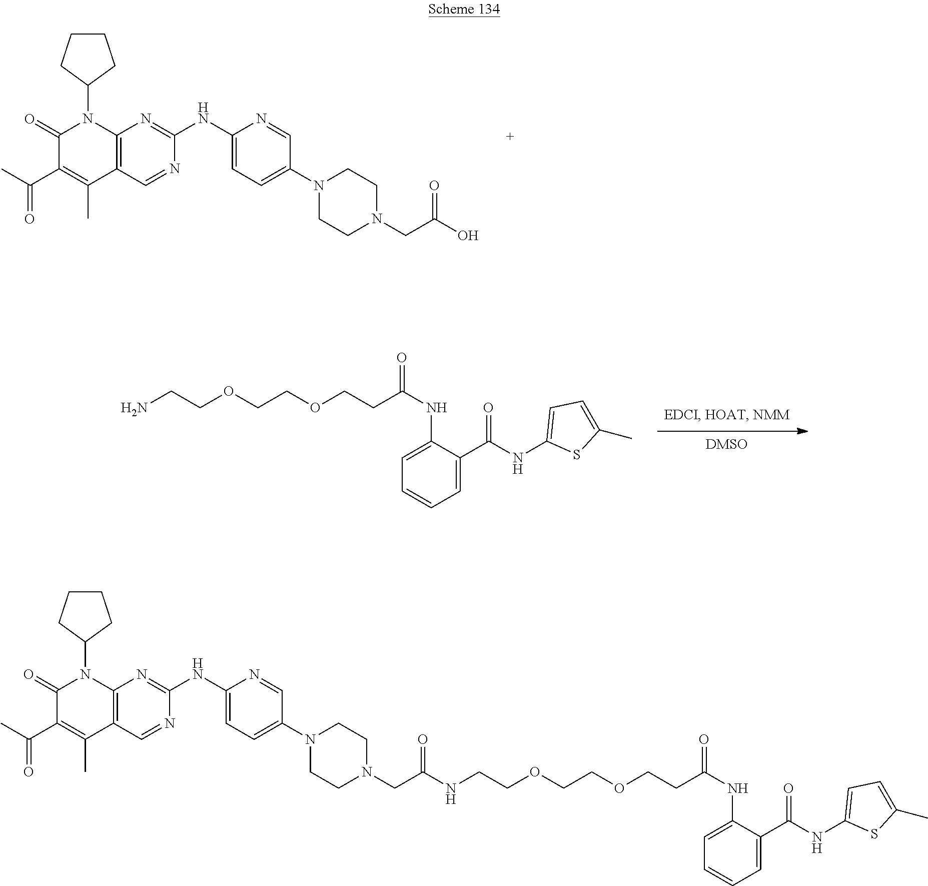

CPD-065 was synthesized following the standard procedure for preparing CPD-008 (14 mg, 27% yield) as a yellow solid. MS (ESI) m/z=879.4 [M+H]⁺.

### Example 135. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methyloxazol-2-yl)benzamide (CPD-066)

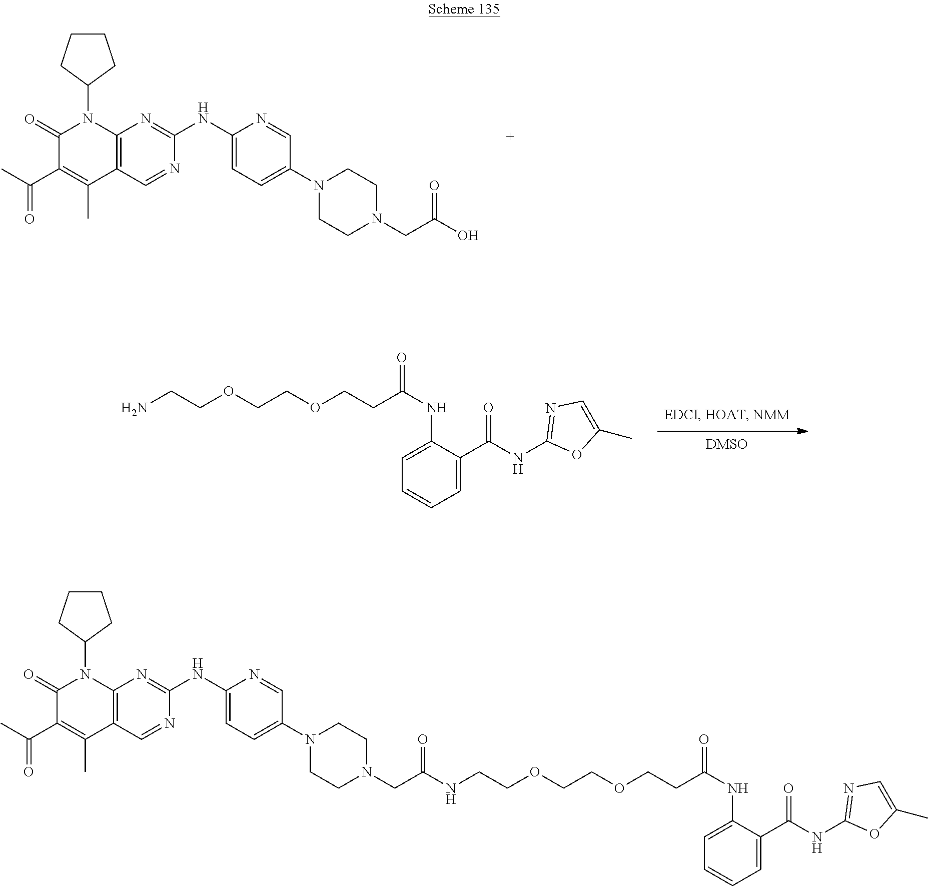

CPD-066 was synthesized following the standard procedure for preparing CPD-008 (6.5 mg, 18% yield) as a yellow solid. MS (ESI) m/z=864.5 [M+H]⁺.

### Example 136. 3-((2-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)ethyl)amino)-N-(1,5-dimethyl-1H-pyrazol-3-yl)-2-methylbenzamide (CPD-067)

### Scheme 136

CPD-067 was synthesized following the standard procedure for preparing CPD-008 (8.7 mg, 47% yield) as a yellow solid. MS (ESI) m/z=863.5 [M+H]⁺.

### Example 137. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(1-methyl-1H-pyrazol-3-yl)benzamide (CPD-068)

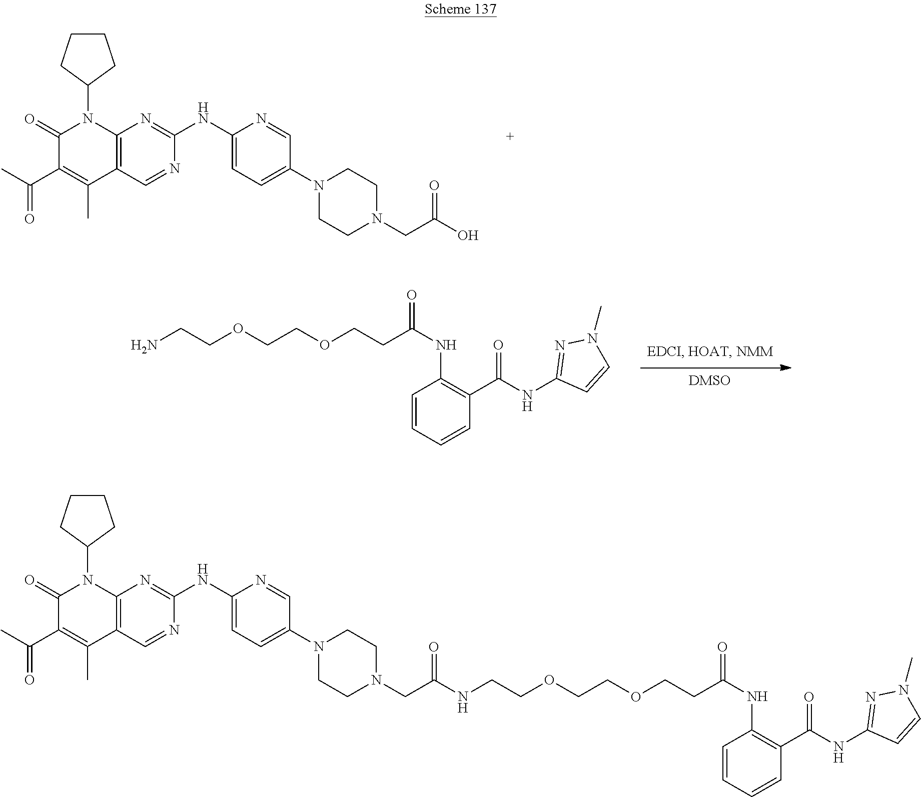

CPD-068 was synthesized following the standard procedure for preparing CPD-008 (8.2 mg, 45% yield) as a yellow solid. MS (ESI) m/z=863.5 [M+H]⁺.

### Example 138. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(1-methyl-5-(trifluoromethyl)-1H-pyrazol-3-yl)benzamide (CPD-069)

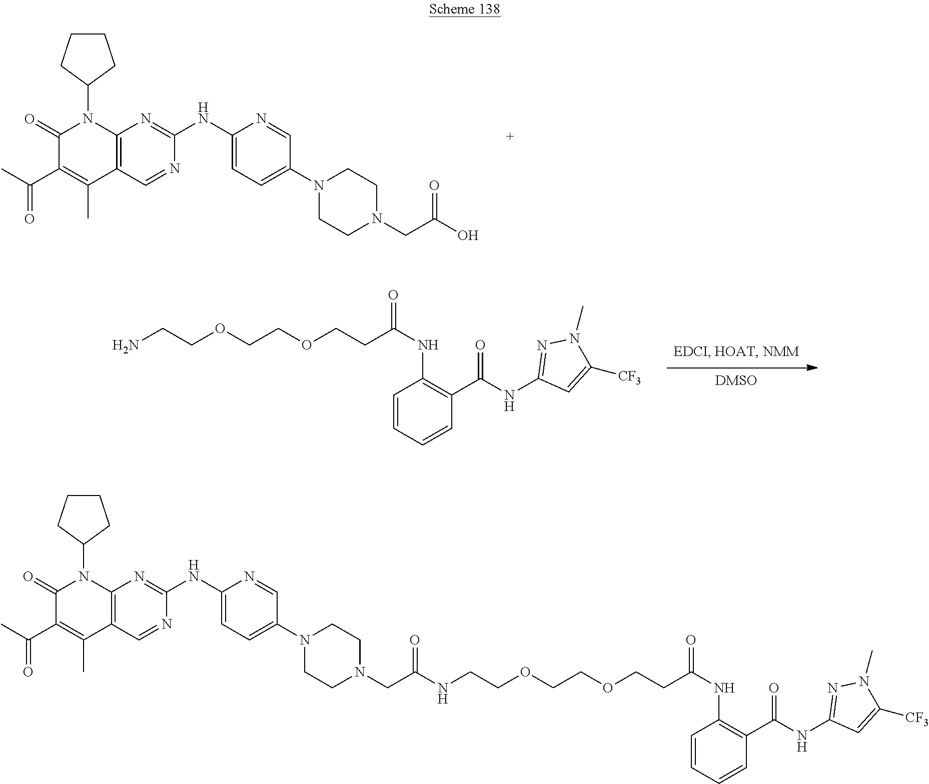

CPD-069 was synthesized following the standard procedure for preparing CPD-008 (6.6 mg, 39% yield) as a yellow solid. MS (ESI) m/z=931.5 [M+H]⁺.

### Example 139. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4-isopropyl-5-methylthiazol-2-yl)benzamide (CPD-070)

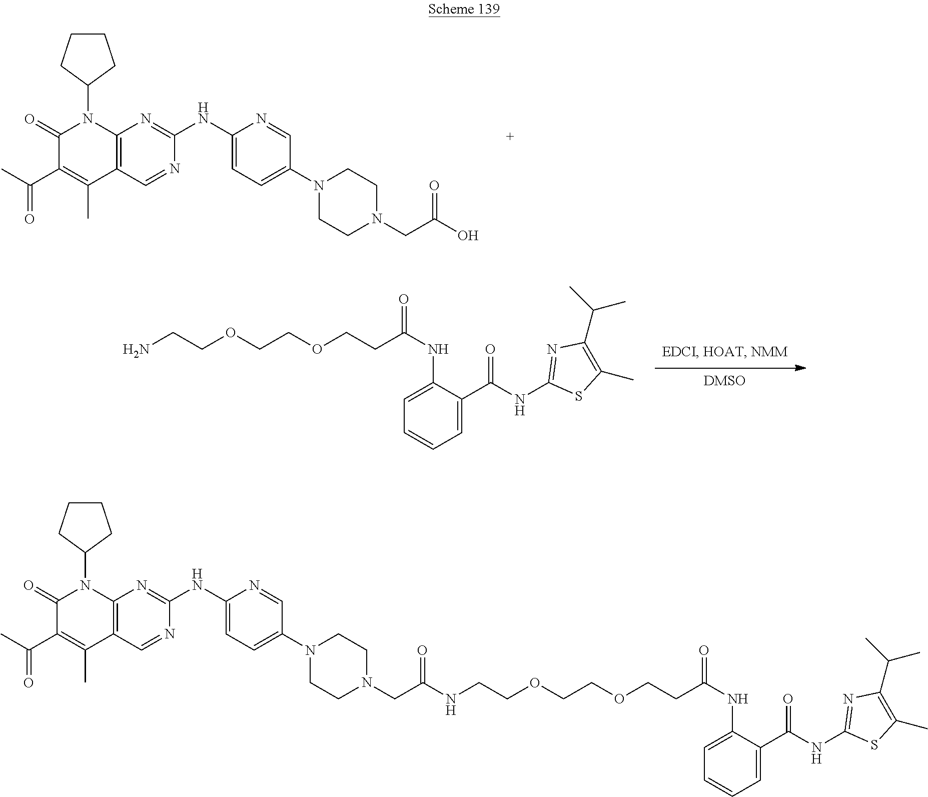

CPD-070 was synthesized following the standard procedure for preparing CPD-008 (2.2 mg, 4% yield) as a yellow solid. MS (ESI) m/z=922.5 [M+H]⁺.

### Example 140. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4-bromo-5-methylthiazol-2-yl)benzamide (CPD-071)

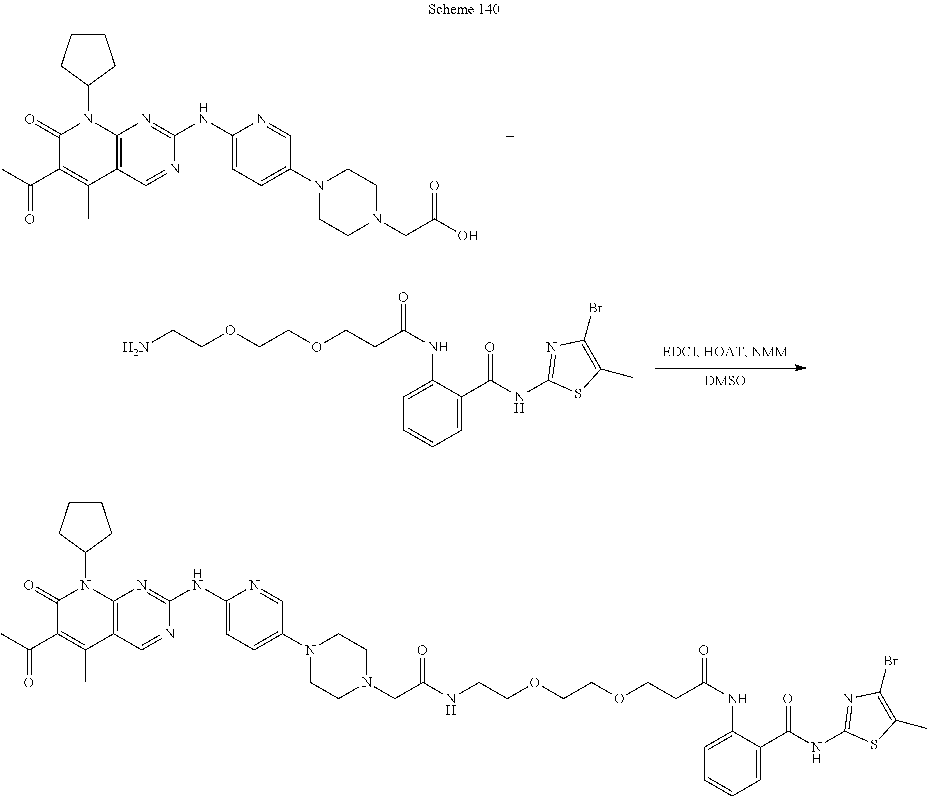

CPD-071 was synthesized following the standard procedure for preparing CPD-008 (38 mg, 37% yield) as a yellow solid. MS (ESI) m/z=958.3 [M+H]⁺.

### Example 141. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methyl-4-(piperidin-4-yl)thiazol-2-yl)benzamide (CPD-072)

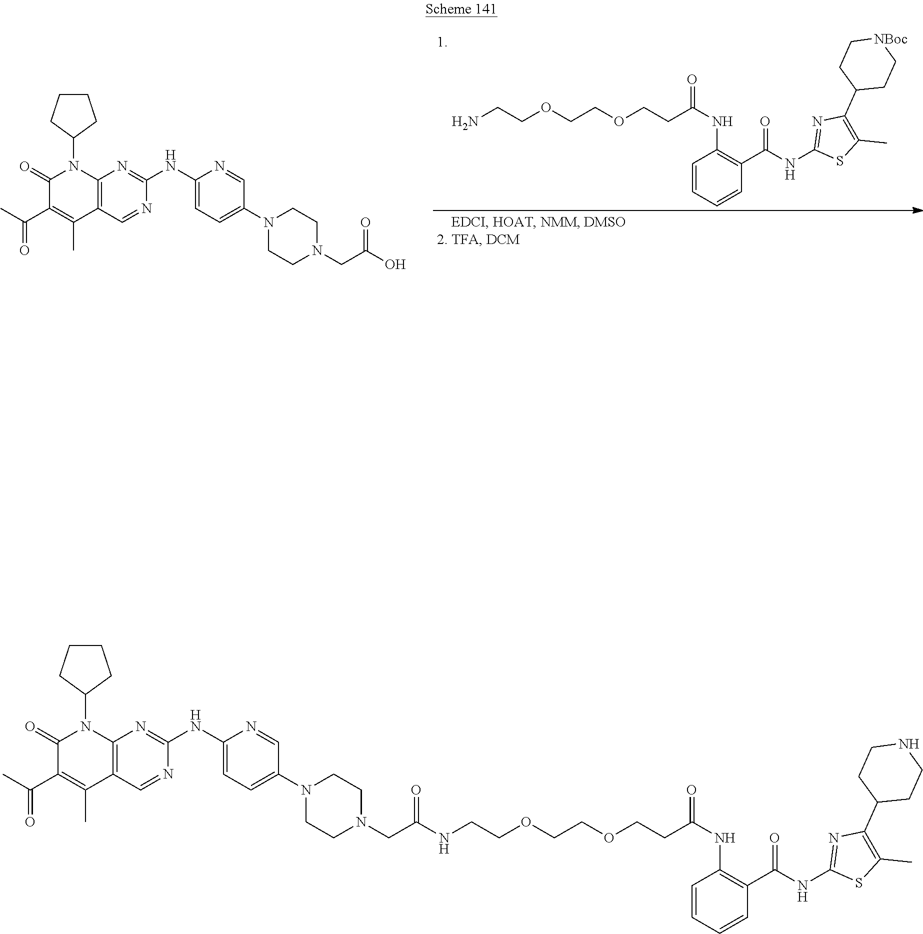 [SMILES: CC(=O)c1c(C)c2cnc(Nc3ccc(N4CCN(CC(=O)O)CC4)cn3)nc2n(C2CCCC2)c1=O ; Cc1sc(NC(=O)c2ccccc2NC(=O)CCOCCOCCN)nc1C1CCN(C(=O)OC(C)(C)C)CC1 ; CC(=O)c1c(C)c2cnc(Nc3ccc(N4CCN(CC(=O)NCCOCCOCCC(=O)Nc5ccccc5C(=O)Nc5nc(C6CCNCC6)c(C)s5)CC4)cn3)nc2n(C2CCCC2)c1=O]

### Step 1. Synthesis of tert-butyl 4-(2-(2-(3-(2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)benzamido)-5-methylthiazol-4-yl)piperidine-1-carboxylate

The title compound was synthesized following the standard procedure for preparing CPD-008 (22 mg, 54% yield) as a yellow solid. MS (ESI) m/z=1063.6 [M+H]⁺.

### Step 2. Synthesis of 2-(3-(2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methyl-4-(piperidin-4-yl)thiazol-2-yl)benzamide

To a solution of tert-butyl 4-(2-(2-(3-(2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)benzamido)-5-methylthiazol-4-yl)piperidine-1-carboxylate (22 mg, 0.021 mmol) in DCM (2 mL) was added TFA (1 mL) at 0° C. After the reaction mixture was stirred at rt for 1 h, it was concentrated under reduced pressure. The residue was purified by reverse-phase chromatography to provide the desired product (5.6 mg, 25% yield) as a yellow solid.

### Example 142. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(1H-pyrazol-3-yl)benzamide (CPD-073)

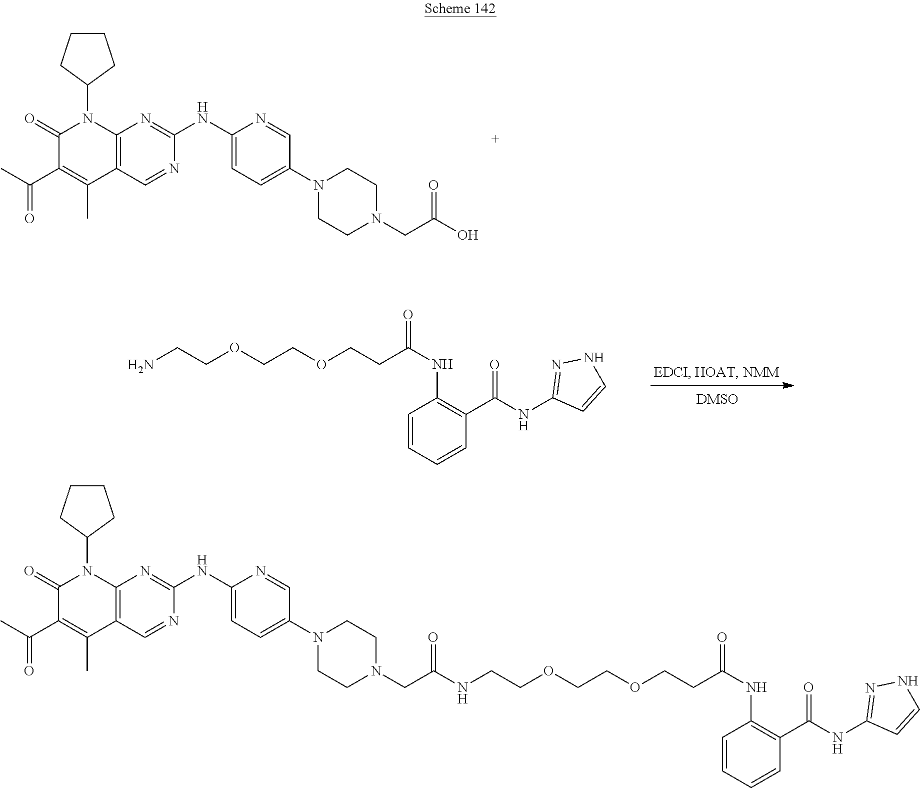 [SMILES: CC(=O)c1c(C)c2cnc(Nc3ccc(N4CCN(CC(=O)O)CC4)cn3)nc2n(C2CCCC2)c1=O ; "+" ; NCCOCCOCCC(=O)Nc1ccccc1C(=O)Nc1cc[nH]n1 ; CC(=O)c1c(C)c2cnc(Nc3ccc(N4CCN(CC(=O)NCCOCCOCCC(=O)Nc5ccccc5C(=O)Nc5cc[nH]n5)CC4)cn3)nc2n(C2CCCC2)c1=O]

CPD-073 was synthesized following the standard procedure for preparing CPD-008 (6.4 mg, 40% yield) as a yellow solid. MS (ESI) m/z=849.5 [M+H]⁺.

### Example 143. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methyl-1H-pyrazol-3-yl)benzamide (CPD-074)

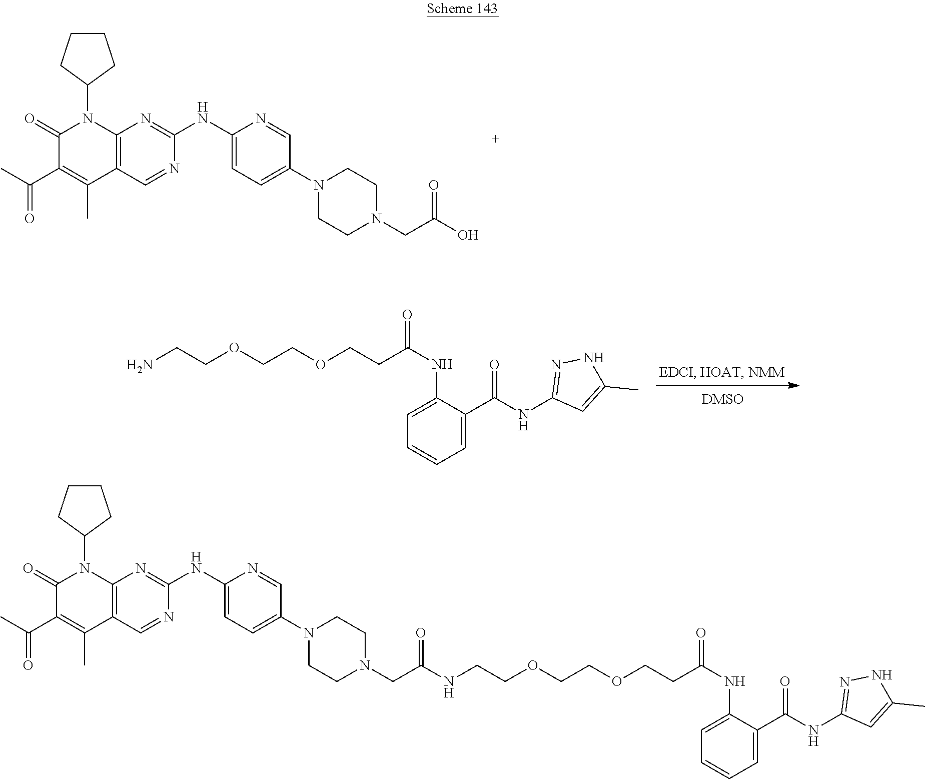 [SMILES: CC(=O)c1c(C)c2cnc(Nc3ccc(N4CCN(CC(=O)O)CC4)cn3)nc2n(C2CCCC2)c1=O ; "+" ; Cc1cc(NC(=O)c2ccccc2NC(=O)CCOCCOCCN)n[nH]1 ; CC(=O)c1c(C)c2cnc(Nc3ccc(N4CCN(CC(=O)NCCOCCOCCC(=O)Nc5ccccc5C(=O)Nc5cc(C)[nH]n5)CC4)cn3)nc2n(C2CCCC2)c1=O]

CPD-074 was synthesized following the standard procedure for preparing CPD-008 (19.7 mg, 34% yield) as a yellow solid. MS (ESI) m/z=863.5 [M+H]⁺.

### Example 144. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4-ethyl-5-methylthiazol-2-yl)benzamide (CPD-075)

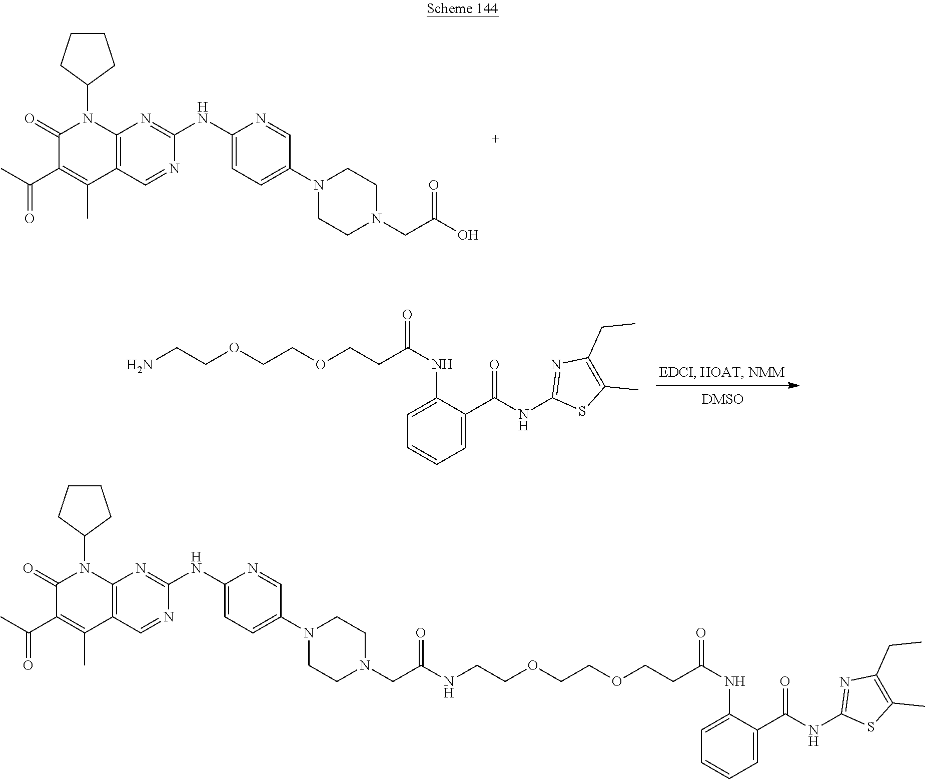 [SMILES: CC(=O)c1c(C)c2cnc(Nc3ccc(N4CCN(CC(=O)O)CC4)cn3)nc2n(C2CCCC2)c1=O ; "+" ; CCc1nc(NC(=O)c2ccccc2NC(=O)CCOCCOCCN)sc1C ; CCc1nc(NC(=O)c2ccccc2NC(=O)CCOCCOCCNC(=O)CN2CCN(c3ccc(Nc4ncc5c(C)c(C(C)=O)c(=O)n(C6CCCC6)c5n4)nc3)CC2)sc1C]

CPD-075 was synthesized following the standard procedure for preparing CPD-008 (30.6 mg, 47% yield) as a yellow solid. MS (ESI) m/z=908.5 [M+H]⁺.

### Example 145. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(1-isopropyl-5-methyl-1H-pyrazol-3-yl)benzamide (CPD-076)

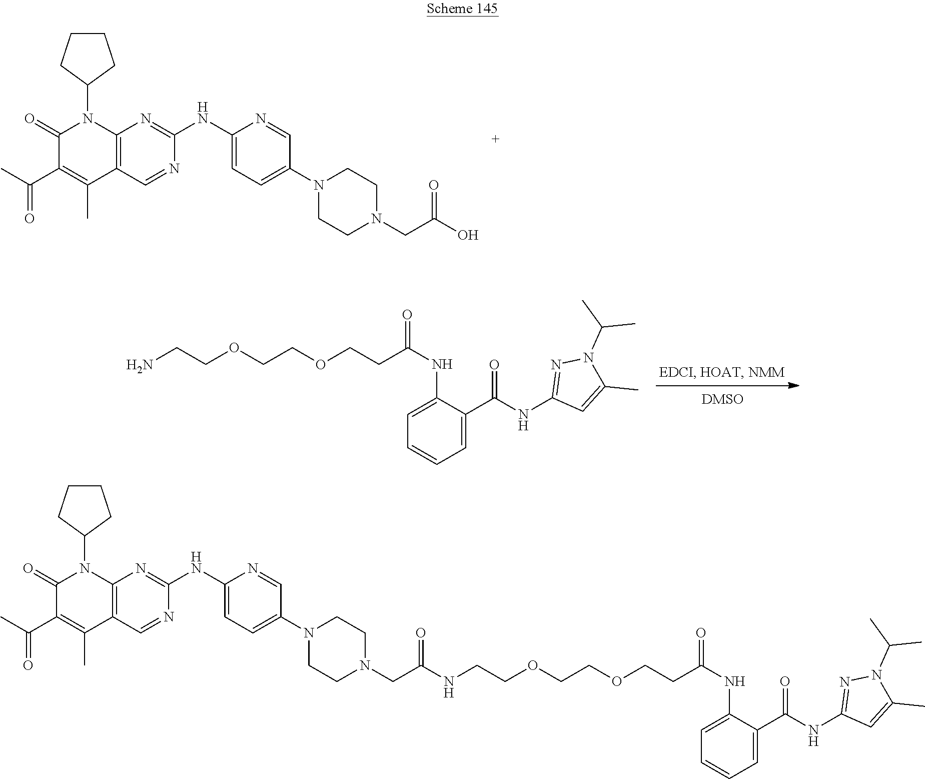 [SMILES: CC(=O)c1c(C)c2cnc(Nc3ccc(N4CCN(CC(=O)O)CC4)cn3)nc2n(C2CCCC2)c1=O ; "+" ; Cc1cc(NC(=O)c2ccccc2NC(=O)CCOCCOCCN)nn1C(C)C ; CC(=O)c1c(C)c2cnc(Nc3ccc(N4CCN(CC(=O)NCCOCCOCCC(=O)Nc5ccccc5C(=O)Nc5cc(C)n(C(C)C)n5)CC4)cn3)nc2n(C2CCCC2)c1=O]

CPD-076 was synthesized following the standard procedure for preparing CPD-008 (13.2 mg, 76% yield) as a yellow solid. MS (ESI) m/z=905.5 [M+H]⁺.

### Example 146. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methyl-4-(trifluoromethyl)thiazol-2-yl)benzamide (CPD-077)

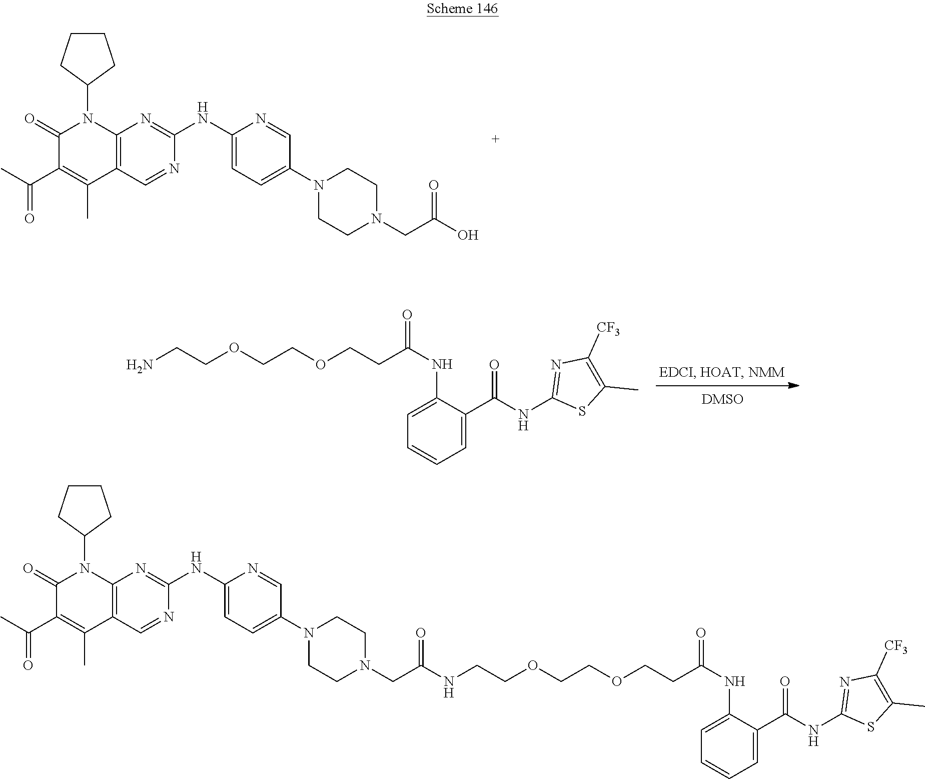 [SMILES: CC(=O)c1c(C)c2cnc(Nc3ccc(N4CCN(CC(=O)O)CC4)cn3)nc2n(C2CCCC2)c1=O ; "+" ; Cc1sc(NC(=O)c2ccccc2NC(=O)CCOCCOCCN)nc1C(F)(F)F ; CC(=O)c1c(C)c2cnc(Nc3ccc(N4CCN(CC(=O)NCCOCCOCCC(=O)Nc5ccccc5C(=O)Nc5nc(C(F)(F)F)c(C)s5)CC4)cn3)nc2n(C2CCCC2)c1=O]

CPD-077 was synthesized following the standard procedure for preparing CPD-008 (16.1 mg, 77% yield) as a yellow solid. MS (ESI) m/z=948.4 [M+H]⁺.

### Example 147. 3-((10-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)decyl)amino)-N-(4,5-dimethylthiazol-2-yl)-2-methylbenzamide (CPD-078)

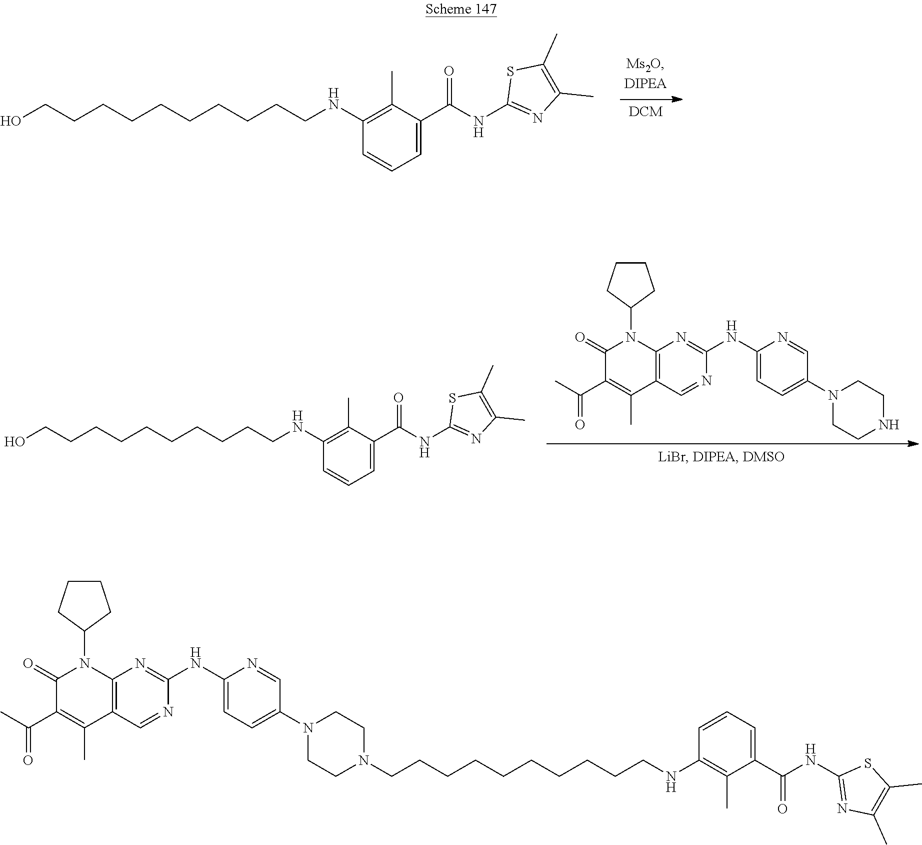

### Step 1. Synthesis of 10-((3-((4,5-dimethylthiazol-2-yl)carbamoyl)-2-methylphenyl)amino)decyl methanesulfonate

To a solution of N-(4,5-dimethylthiazol-2-yl)-3-((10-hydroxydecyl)amino)-2-methylbenzamide (10 mg, 0.02 mmol) and DIPEA (13 mg, 0.1 mmol) in DCM (5 mL) was added Ms₂O (7 mg, 0.04 mmol) at 0° C. After the reaction mixture was stirred at rt for 1 h, it was poured into water (10 mL) and extracted with DCM (3×10 mL). The combined organic layers were washed with brine, dried over anhydrous sodium sulfate, filtered and concentrated under reduced pressure. The resulting residue was used in the next step directly without further purification. MS (ESI) m/z=496.2 [M+H]⁺.

### Step 2. Synthesis of 3-((10-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)decyl)amino)-N-(4,5-dimethylthiazol-2-yl)-2-methylbenzamide

To a solution of 10-((3-((4,5-dimethylthiazol-2-yl)carbamoyl)-2-methylphenyl)amino)decyl methanesulfonate (10 mg, 0.02 mmol) and 6-acetyl-8-cyclopentyl-5-methyl-2-((5-(piperazin-1-yl)pyridin-2-yl)amino)pyrido[2,3-d]pyrimidin-7(8H)-one (8.9 mg, 0.02 mmol) in DMSO (2 mL) were added LiBr (5 mg, 0.04 mmol) and DIPEA (13 mg, 0.1 mmol) at rt. The reaction mixture was stirred at 100° C. for 1 h. After cooling down to rt, the mixture was purified by reverse-phase chromatography and prep-TLC to provide the desired product (3.8 mg, 22% yield) as a yellow solid. MS (ESI) m/z=847.5 [M+H]⁺.

### Example 148. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-cyclopropyl-1-methyl-1H-pyrazol-3-yl)benzamide (CPD-079)

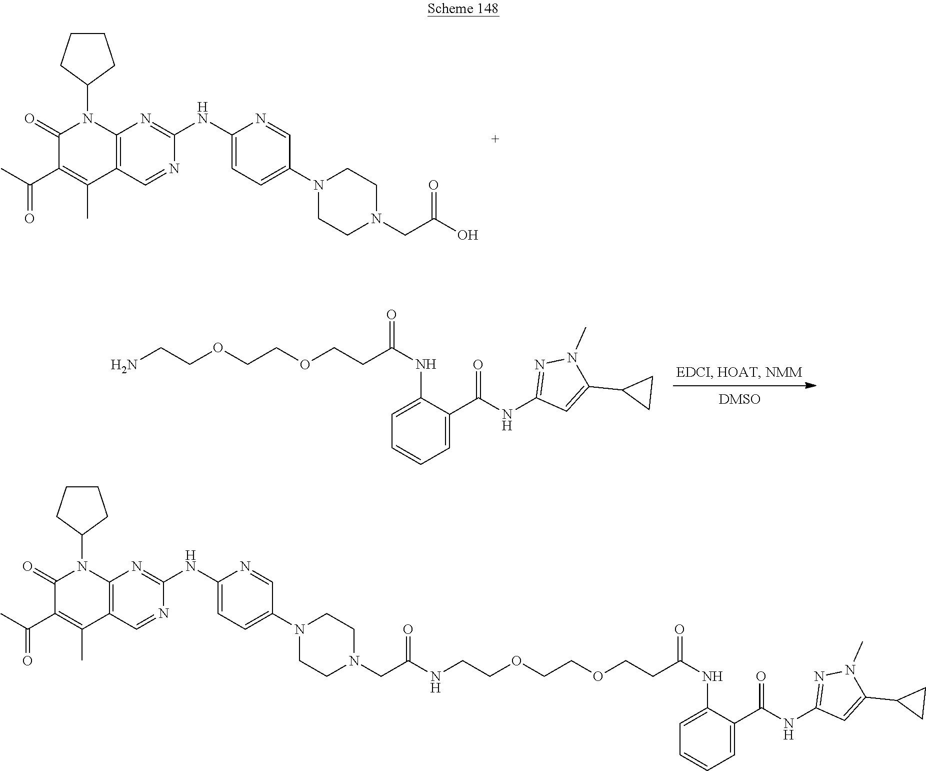 [SMILES: CC(=O)c1c(C)c2cnc(Nc3ccc(N4CCN(CC(=O)O)CC4)cn3)nc2n(C2CCCC2)c1=O ; "+" ; Cn1nc(NC(=O)c2ccccc2NC(=O)CCOCCOCCN)cc1C1CC1 ; CC(=O)c1c(C)c2cnc(Nc3ccc(N4CCN(CC(=O)NCCOCCOCCC(=O)Nc5ccccc5C(=O)Nc5cc(C6CC6)n(C)n5)CC4)cn3)nc2n(C2CCCC2)c1=O]

CPD-079 was synthesized following the standard procedure for preparing CPD-008 (7.5 mg, 38% yield) as a yellow solid. MS (ESI) m/z=903.5 [M+H]⁺.

### Example 149. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methyl-4-(1-methylpiperidin-4-yl)thiazol-2-yl)benzamide (CPD-080)

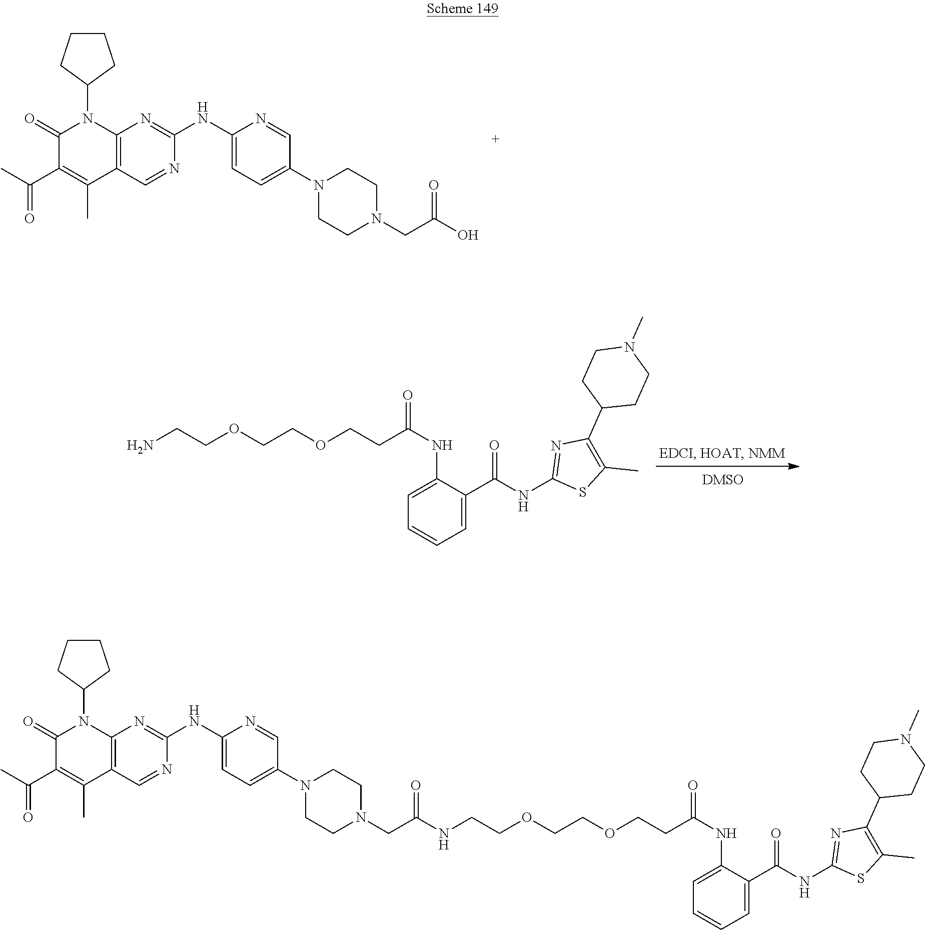 [SMILES: CC(=O)c1c(C)c2cnc(Nc3ccc(N4CCN(CC(=O)O)CC4)cn3)nc2n(C2CCCC2)c1=O ; "+" ; Cc1sc(NC(=O)c2ccccc2NC(=O)CCOCCOCCN)nc1C1CCN(C)CC1 ; CC(=O)c1c(C)c2cnc(Nc3ccc(N4CCN(CC(=O)NCCOCCOCCC(=O)Nc5ccccc5C(=O)Nc5nc(C6CCN(C)CC6)c(C)s5)CC4)cn3)nc2n(C2CCCC2)c1=O]

CPD-080 was synthesized following the standard procedure for preparing CPD-008 (3.3 mg, 16% yield) as a yellow solid. MS (ESI) m/z=977.5 [M+H]⁺.

### Example 150. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-fluoropyridin-2-yl)benzamide (CPD-081)

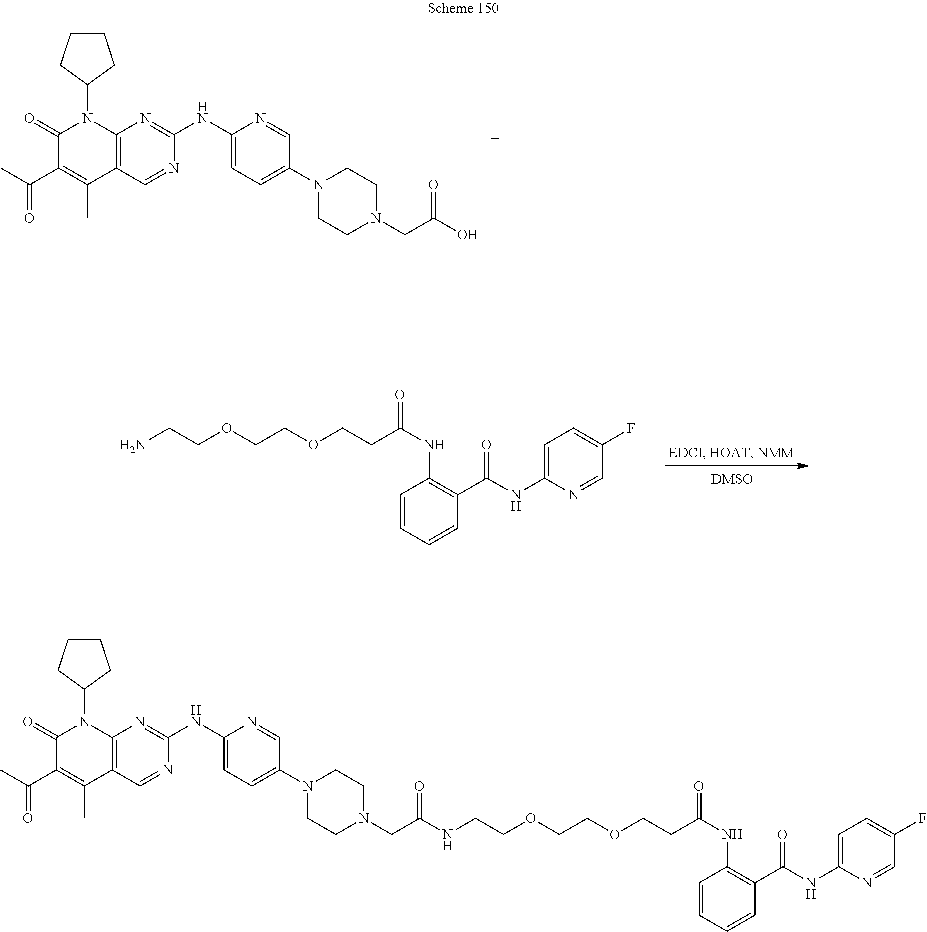 [SMILES: CC(=O)c1c(C)c2cnc(Nc3ccc(N4CCN(CC(=O)O)CC4)cn3)nc2n(C2CCCC2)c1=O ; "+" ; NCCOCCOCCC(=O)Nc1ccccc1C(=O)Nc1ccc(F)cn1 ; CC(=O)c1c(C)c2cnc(Nc3ccc(N4CCN(CC(=O)NCCOCCOCCC(=O)Nc5ccccc5C(=O)Nc5ccc(F)cn5)CC4)cn3)nc2n(C2CCCC2)c1=O]

CPD-081 was synthesized following the standard procedure for preparing CPD-008 (25.3 mg, 39% yield) as a yellow solid. MS (ESI) m/z=878.5 [M+H]⁺.

### Example 151. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-chloropyridin-2-yl)benzamide (CPD-082)

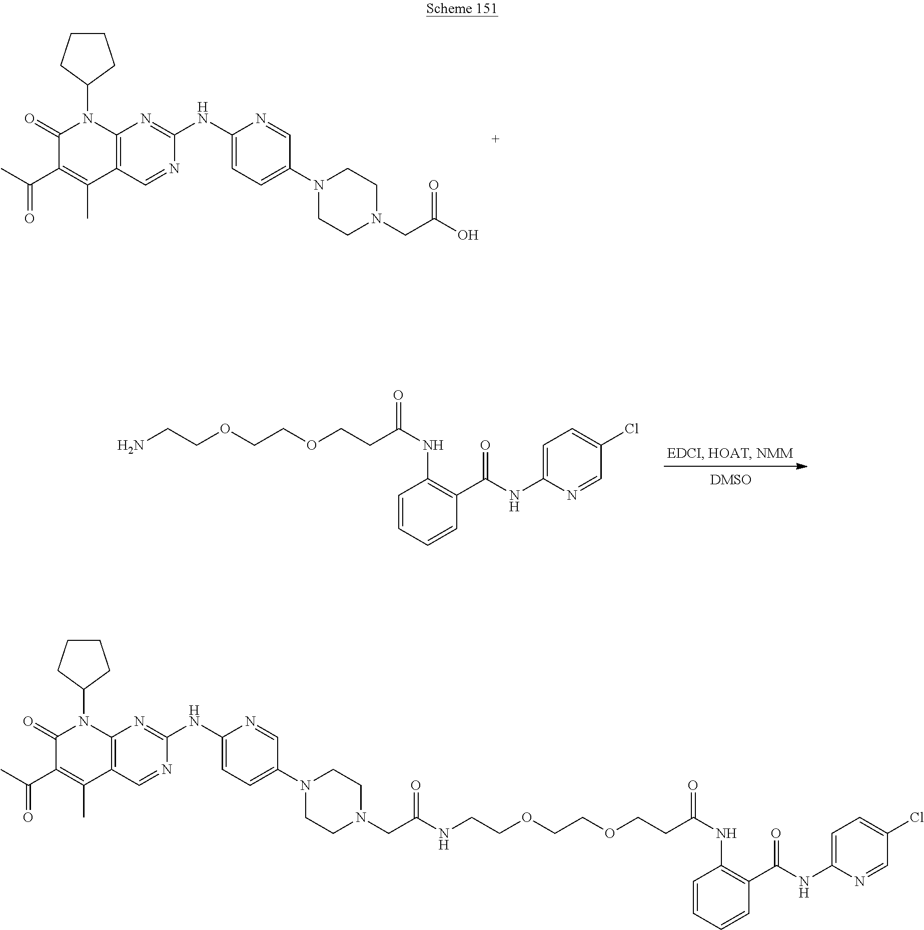 [SMILES: CC(=O)c1c(C)c2cnc(Nc3ccc(N4CCN(CC(=O)O)CC4)cn3)nc2n(C2CCCC2)c1=O ; "+" ; NCCOCCOCCC(=O)Nc1ccccc1C(=O)Nc1ccc(Cl)cn1 ; CC(=O)c1c(C)c2cnc(Nc3ccc(N4CCN(CC(=O)NCCOCCOCCC(=O)Nc5ccccc5C(=O)Nc5ccc(Cl)cn5)CC4)cn3)nc2n(C2CCCC2)c1=O]

CPD-082 was synthesized following the standard procedure for preparing CPD-008 (10.1 mg, 13% yield) as a yellow solid. MS (ESI) m/z=894.5 [M+H]⁺.

### Example 152. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-cyanopyridin-2-yl)benzamide (CPD-083)

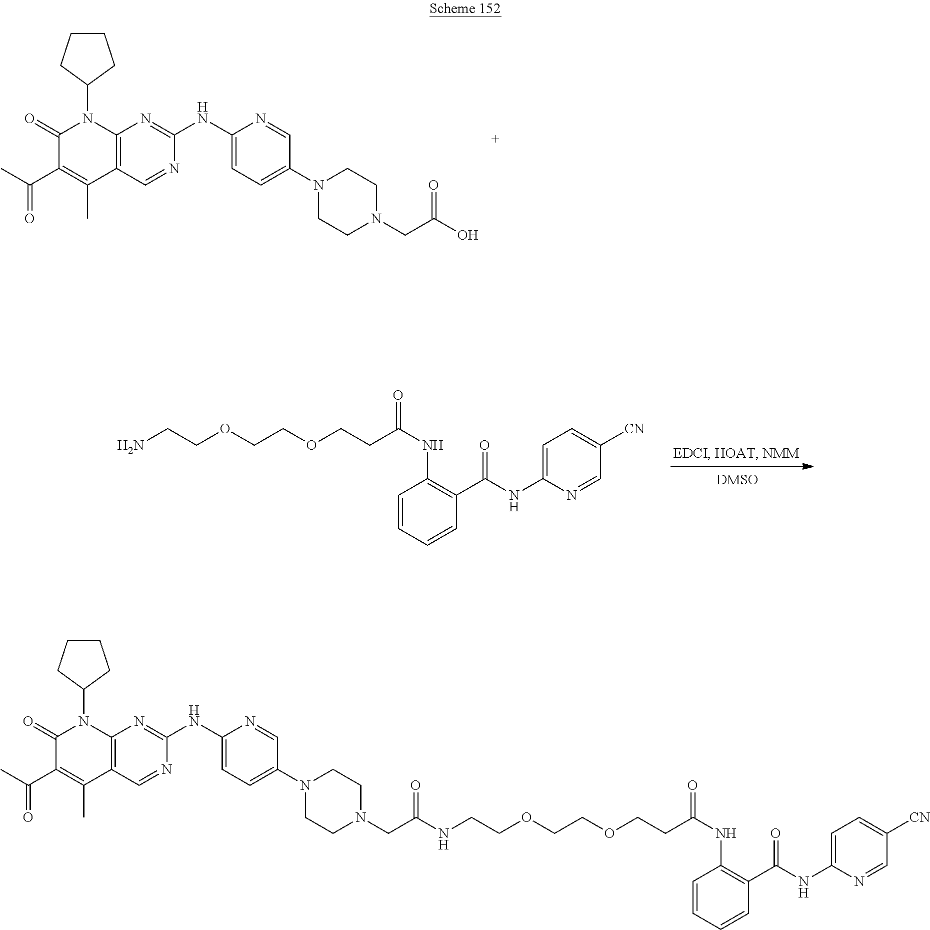 [SMILES: CC(=O)c1c(C)c2cnc(Nc3ccc(N4CCN(CC(=O)O)CC4)cn3)nc2n(C2CCCC2)c1=O ; "+" ; N#Cc1ccc(NC(=O)c2ccccc2NC(=O)CCOCCOCCN)nc1 ; CC(=O)c1c(C)c2cnc(Nc3ccc(N4CCN(CC(=O)NCCOCCOCCC(=O)Nc5ccccc5C(=O)Nc5ccc(C#N)cn5)CC4)cn3)nc2n(C2CCCC2)c1=O]

CPD-083 was synthesized following the standard procedure for preparing CPD-008 (23.5 mg, 46% yield) as a yellow solid. MS (ESI) m/z=885.4 [M+H]⁺.

### Example 153. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-(trifluoromethyl)pyridin-2-yl)benzamide (CPD-084)

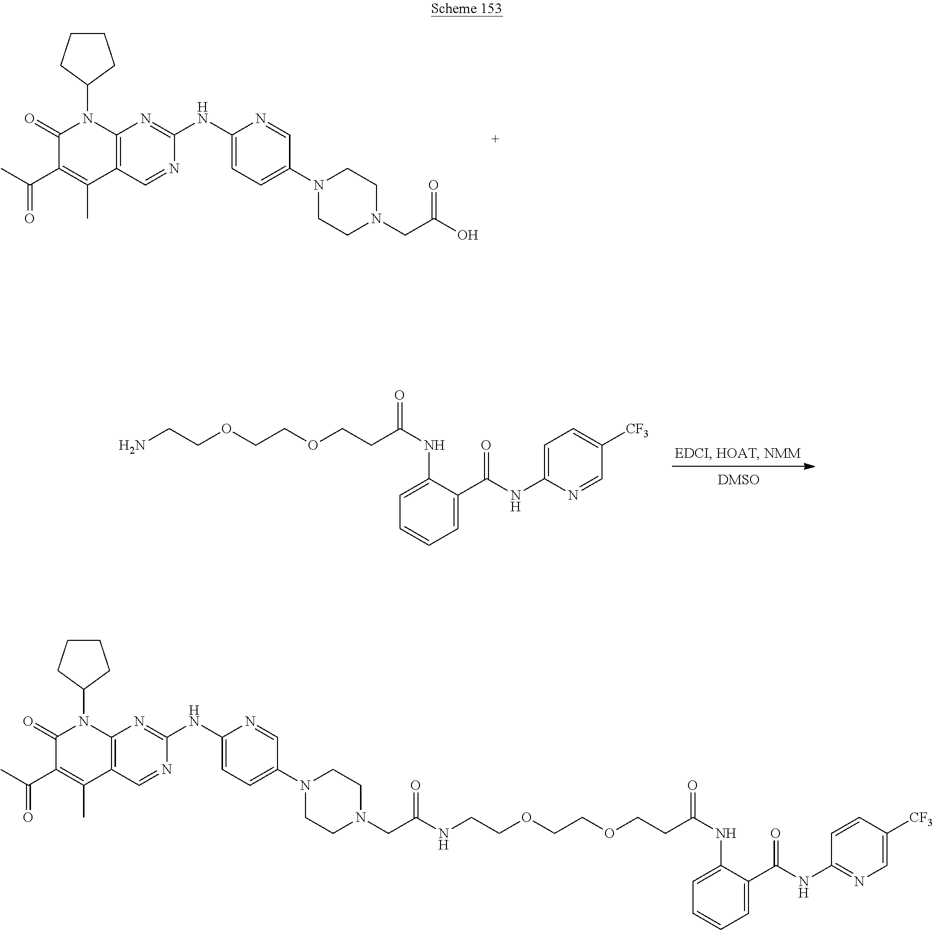 [SMILES: CC(=O)c1c(C)c2cnc(Nc3ccc(N4CCN(CC(=O)O)CC4)cn3)nc2n(C2CCCC2)c1=O ; "+" ; NCCOCCOCCC(=O)Nc1ccccc1C(=O)Nc1ccc(C(F)(F)F)cn1 ; CC(=O)c1c(C)c2cnc(Nc3ccc(N4CCN(CC(=O)NCCOCCOCCC(=O)Nc5ccccc5C(=O)Nc5ccc(C(F)(F)F)cn5)CC4)cn3)nc2n(C2CCCC2)c1=O]

CPD-084 was synthesized following the standard procedure for preparing CPD-008 (14.0 mg, 41% yield) as a yellow solid. MS (ESI) m/z=928.4 [M+H]⁺.

### Example 154. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(6-methoxypyridazin-3-yl)benzamide (CPD-085)

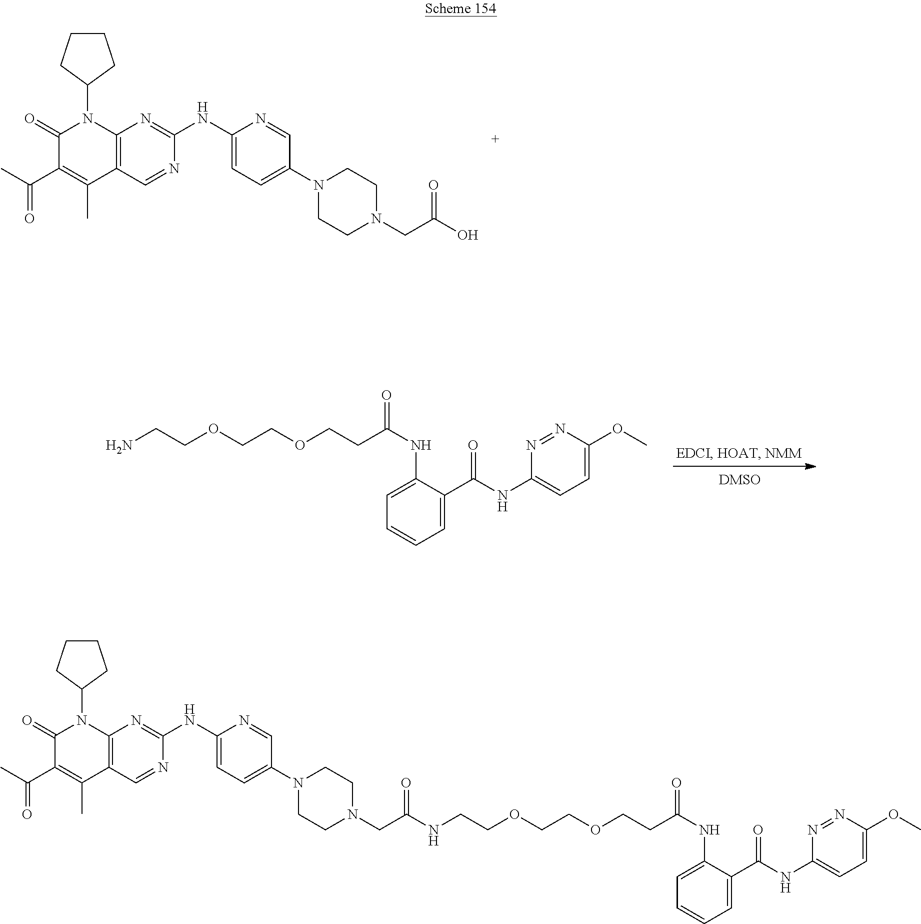 [SMILES: CC(=O)c1c(C)c2cnc(Nc3ccc(N4CCN(CC(=O)O)CC4)cn3)nc2n(C2CCCC2)c1=O ; "+" ; COc1ccc(NC(=O)c2ccccc2NC(=O)CCOCCOCCN)nn1 ; COc1ccc(NC(=O)c2ccccc2NC(=O)CCOCCOCCNC(=O)CN2CCN(c3ccc(Nc4ncc5c(C)c(C(C)=O)c(=O)n(C6CCCC6)c5n4)nc3)CC2)nn1]

CPD-085 was synthesized following the standard procedure for preparing CPD-008 (15.0 mg, 34% yield) as a yellow solid. MS (ESI) m/z=891.5 [M+H]⁺.

### Example 155. 3-((2-(2-(2-((2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)ethyl)amino)ethoxy)ethoxy)ethyl)amino)-N-(4,5-dimethylthiazol-2-yl)-2-methylbenzamide (CPD-086)

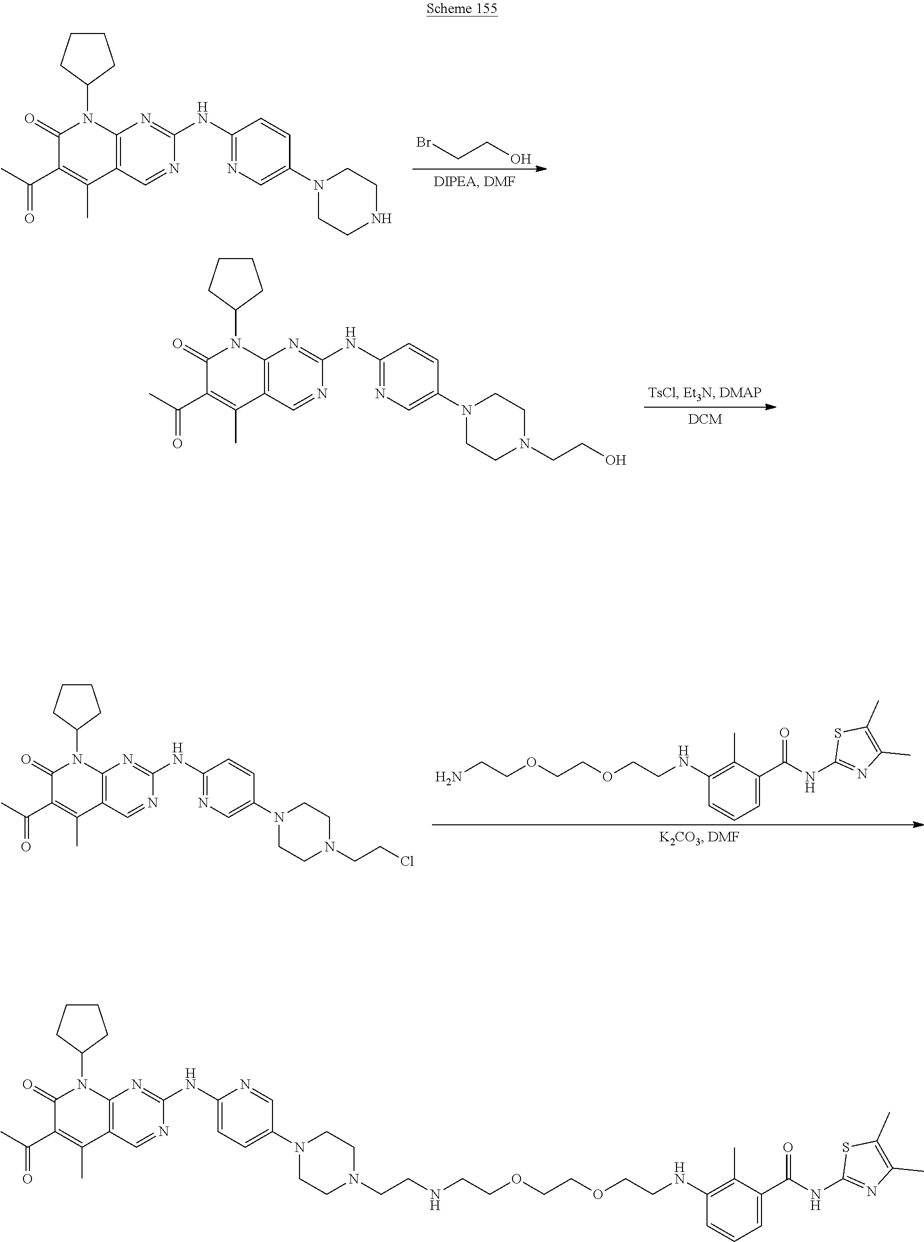

### Step 1. Synthesis of 6-acetyl-8-cyclopentyl-2-((5-(4-(2-hydroxyethyl)piperazin-1-yl)pyridin-2-yl)amino)-5-methylpyrido[2,3-d]pyrimidin-7(8H)-one

To a solution of 6-acetyl-8-cyclopentyl-5-methyl-2-((5-(piperazin-1-yl)pyridin-2-yl)amino)pyrido[2,3-d]pyrimidin-7(8H)-one (400 mg, 0.89 mmol) in DMF (10 mL) were added 2-bromoethan-1-ol (333 mg, 2.68 mmol) and DIEA (176 mg, 0.54 mmol) at rt. The reaction mixture was stirred at 90° C. for 2 h. After cooling down to rt, the reaction mixture was diluted water (50 mL) and extracted with ethyl acetate (3×50 mL). The combined organic layers were washed with brine, dried over anhydrous Na₂SO₄, filtered and concentrated under reduced pressure. The residue was purified by silica gel column chromatography to provide the desired product (400 mg, 91% yield) as a yellow solid. MS (ESI) m/z=492.3 [M+H]⁺.

### Step 2. Synthesis of 6-acetyl-2-((5-(4-(2-chloroethyl)piperazin-1-yl)pyridin-2-yl)amino)-8-cyclopentyl-5-methylpyrido[2,3-d]pyrimidin-7(8H)-one

To a solution of 6-acetyl-8-cyclopentyl-2-((5-(4-(2-hydroxyethyl)piperazin-1-yl)pyridin-2-yl)amino)-5-methylpyrido[2,3-d]pyrimidin-7(8H)-one (100 mg, 0.20 mmol) in DCM (20 mL) were added 4-methylbenzenesulfonyl chloride (40 mg, 0.60 mmol), Et₃N (101 mg, 1.00 mmol) and DMAP (37 mg, 0.30 mmol). After the reaction mixture was stirred at rt for 2 h, it was quenched with water (20 mL) and extracted with DCM (2×30 mL). The combined organic layers were washed with brine, dried over anhydrous Na₂SO₄, filtered and concentrated under reduced pressure. The residue was purified by silica gel column chromatography to provide the desired product (90 mg, 86% yield) as a light-yellow solid. MS (ESI) m/z=510.4 [M+H]⁺.

### Step 3. Synthesis of 3-((2-(2-(2-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)amino)ethoxy)ethoxy)ethyl)amino)-N-(4,5-dimethylthiazol-2-yl)-2-methylbenzamide

To a solution of 6-acetyl-2-((5-(4-(2-chloroethyl)piperazin-1-yl)pyridin-2-yl)amino)-8-cyclopentyl-5-methylpyrido[2,3-d]pyrimidin-7(8H)-one (50 mg, 0.10 mmol) in DMF (5 mL) was added 3-((2-(2-(2-aminoethoxy)ethoxy)ethyl)amino)-N-(4,5-dimethylthiazol-2-yl)-2-methylbenzamide (20 mg, 0.05 mmol) and K₂CO₃ (35 mg, 0.25 mmol) at rt. The reaction mixture was stirred at 90° C. for 4 h. After cooling down to rt, the reaction was diluted with water (20 mL) and extracted with ethyl acetate (3×20 mL). The combined organic layers were washed with brine, dried over anhydrous Na₂SO₄, filtered and concentrated under reduced pressure. The residue was purified by reverse-phase column chromatography to provide the desired product (4 mg, 9% yield) as a yellow solid. MS (ESI) m/z=866.5 [M+H]⁺.

### Example 156. 2-(8-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)octanamido)-N-(5-methylpyridin-2-yl)benzamide (CPD-183)

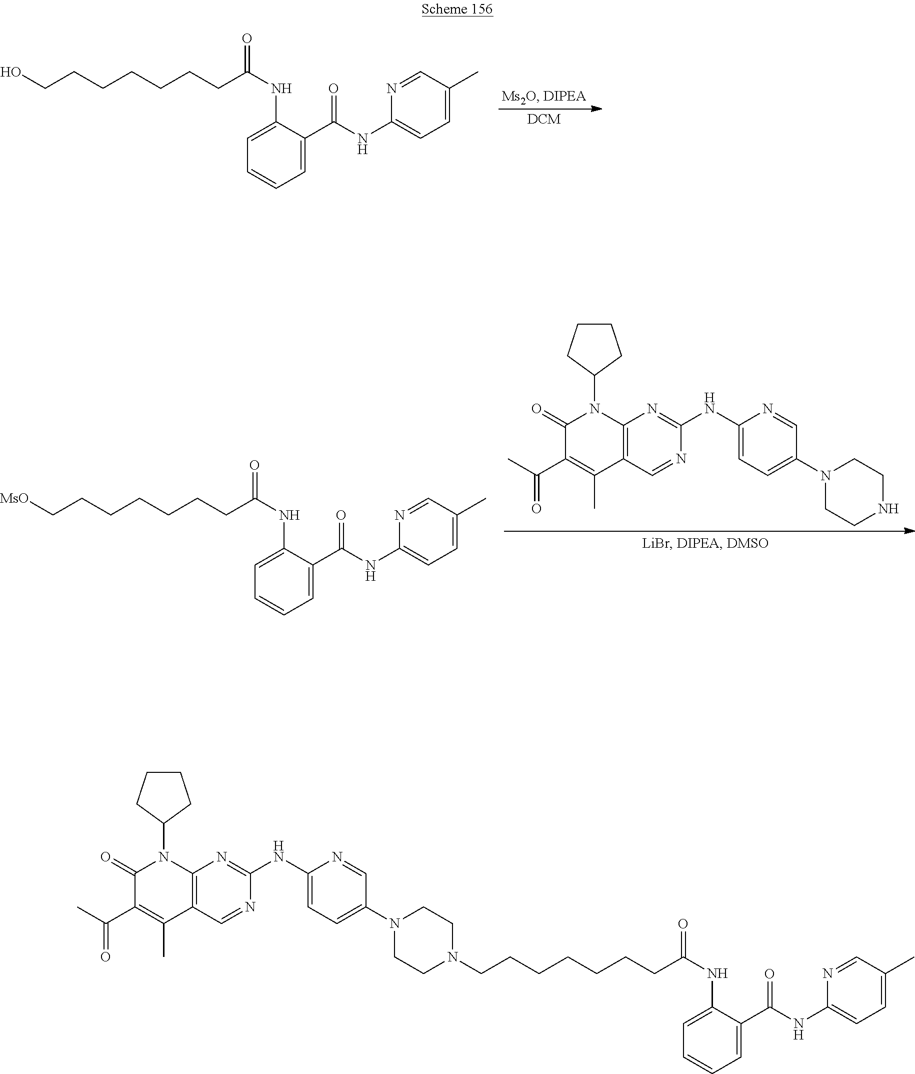

CPD-183 was synthesized following the standard procedure for preparing CPD-078 (13.0 mg, 29% yield over 2 steps) as a yellow solid. MS (ESI) m/z=799.5 [M+H]⁺.

### Example 157. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-cyclopropylpyridin-2-yl)benzamide (CPD-184)

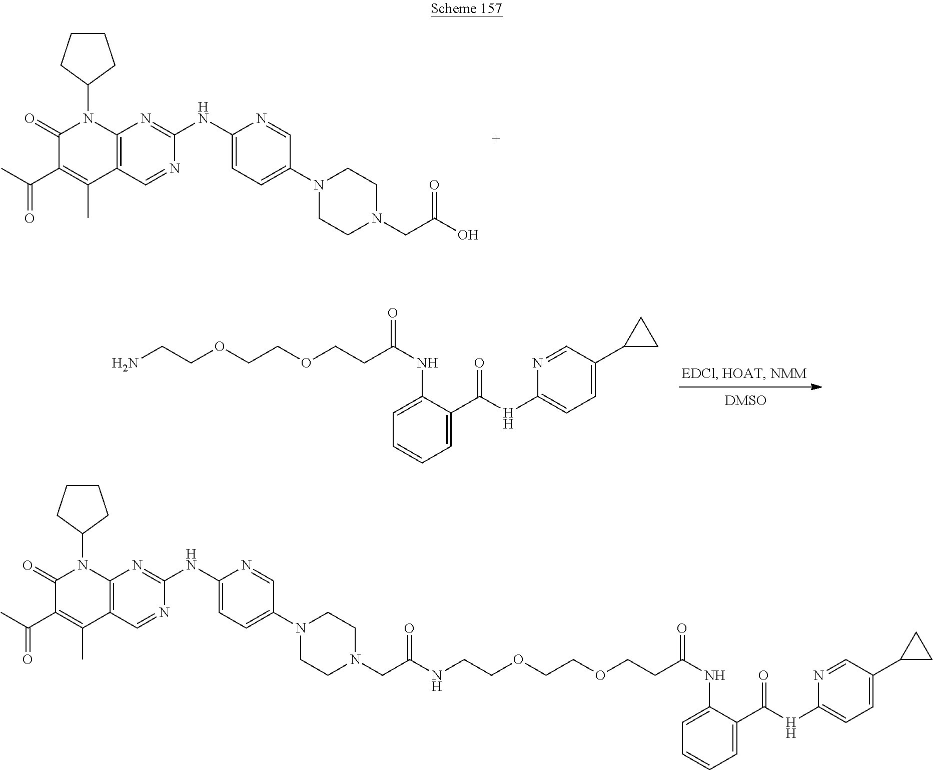

CPD-184 was synthesized following the standard procedure for preparing CPD-008 (17.7 mg, 54% yield) as a yellow solid. MS (ESI) m/z=900.5 [M+H]⁺.

### Example 158. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(6-(dimethylamino)pyridazin-3-yl)benzamide (CPD-185)

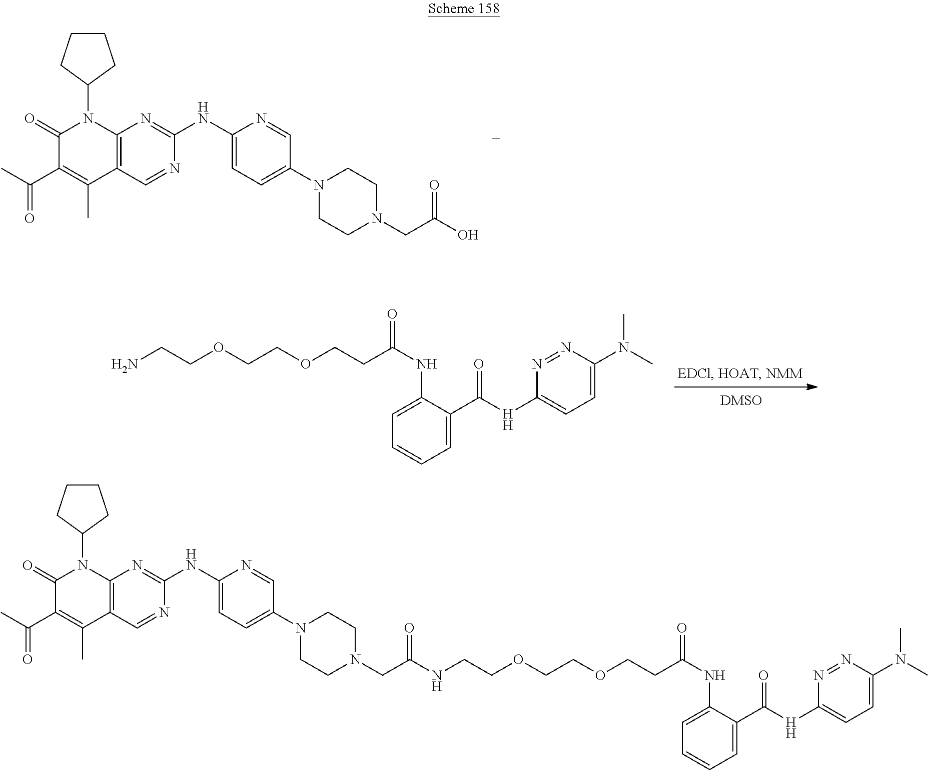

CPD-185 was synthesized following the standard procedure for preparing CPD-008 (3.9 mg, 18% yield) as a yellow solid. MS (ESI) m/z=904.5 [M+H]⁺.

### Example 159. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(2-methylpyrimidin-5-yl)benzamide (CPD-186)

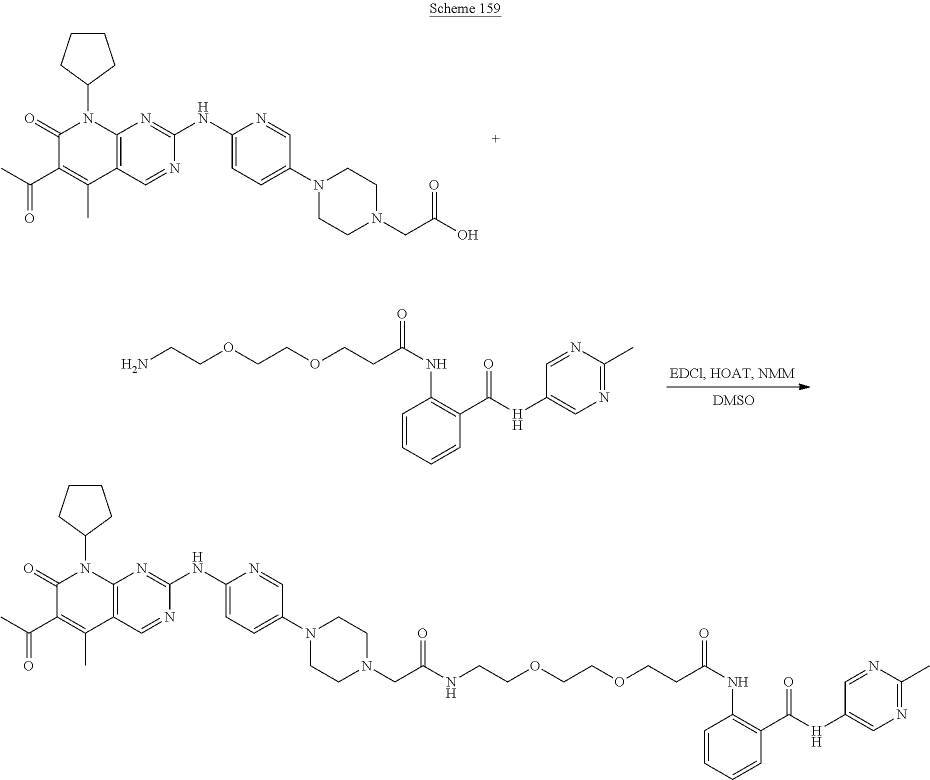

CPD-186 was synthesized following the standard procedure for preparing CPD-008 (22.5 mg, 44% yield) as a yellow solid. MS (ESI) m/z=875.5 [M+H]⁺.

### Example 160. 2-(3-(2-(2-((2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)ethyl)amino)ethoxy)ethoxy)propanamido)-N-(4,5-dimethylthiazol-2-yl)benzamide (CPD-187)

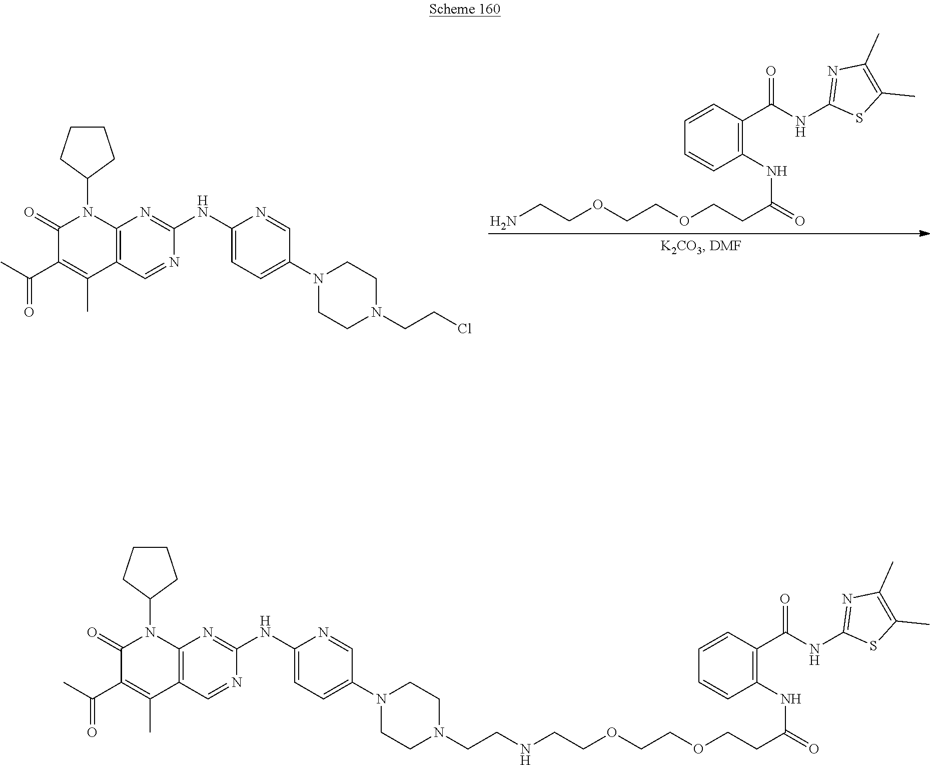 [SMILES: CC(=O)c1c(C)c2cnc(Nc3ccc(N4CCN(CCCl)CC4)cn3)nc2n(C2CCCC2)c1=O ; Cc1nc(NC(=O)c2ccccc2NC(=O)CCOCCOCCN)sc1C ; CC(=O)c1c(C)c2cnc(Nc3ccc(N4CCN(CCNCCOCCOCCC(=O)Nc5ccccc5C(=O)Nc5nc(C)c(C)s5)CC4)cn3)nc2n(C2CCCC2)c1=O]

CPD-187 was synthesized following the standard procedure for preparing CPD-086 (1.6 mg, 7% yield) as a yellow solid. MS (ESI) m/z=880.5 [M+H]⁺.

### Example 161. 3-((7-((2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)ethyl)amino)heptyl)amino)-N-(4,5-dimethylthiazol-2-yl)-2-methylbenzamide (CPD-188)

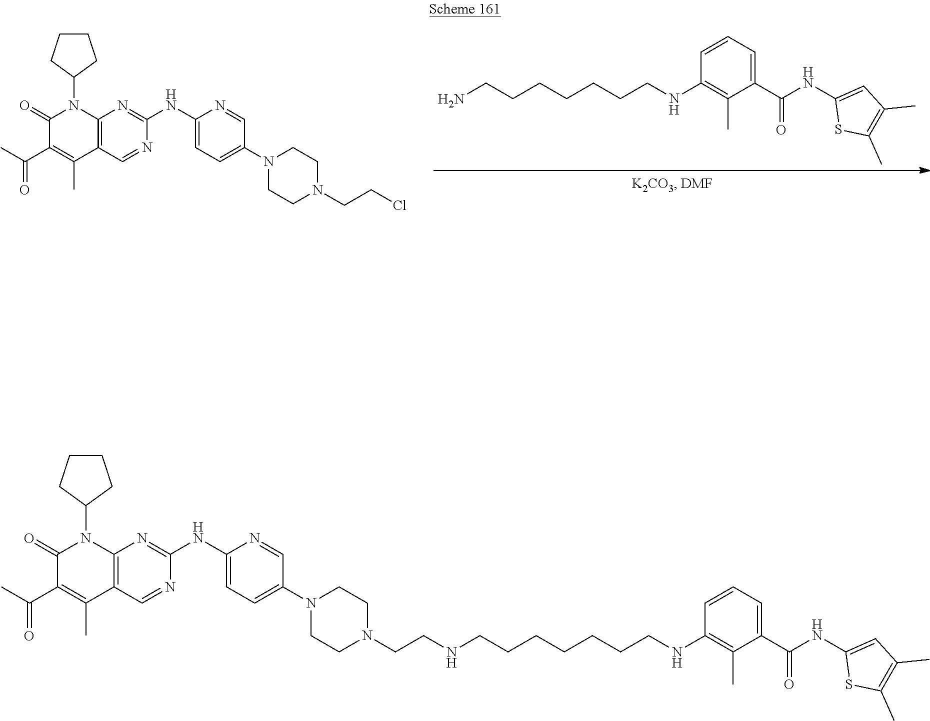 [SMILES: CC(=O)c1c(C)c2cnc(Nc3ccc(N4CCN(CCCl)CC4)cn3)nc2n(C2CCCC2)c1=O ; Cc1cc(NC(=O)c2cccc(NCCCCCCCN)c2C)sc1C ; CC(=O)c1c(C)c2cnc(Nc3ccc(N4CCN(CCNCCCCCCCNc5cccc(C(=O)Nc6cc(C)c(C)s6)c5C)CC4)cn3)nc2n(C2CCCC2)c1=O]

CPD-188 was synthesized following the standard procedure for preparing CPD-086 (2.0 mg, 9% yield) as a yellow solid. MS (ESI) m/z=848.5 [M+H]⁺.

### Example 162. 4-((2-Aminoethyl)amino)-2-methyl-N-(5-methylthiazol-2-yl)benzamide (BL1-81)

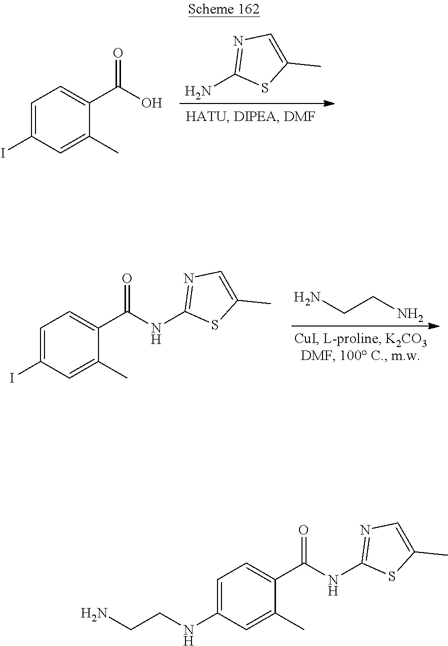

### Step 1. Synthesis of 4-iodo-2-methyl-N-(5-methylthiazol-2-yl)benzamide

To a solution of 4-iodo-2-methylbenzoic acid (6 g, 22.9 mmol) and 5-methylthiazol-2-amine (2.75 g, 24.0 mmol) in DMF (50 ml) were added DIPEA (5.9 g, 45.8 mmol) and HATU (10.4 g, 27.5 mmol) at rt. The reaction mixture was stirred at 80° C. for 2 h. After cooling down to rt, the solution was poured into water (200 mL) and extracted with ethyl acetate (100 mL×3). The combined organic layers were washed with brine, dried over anhydrous Na₂SO₄, filtered and concentrated under reduced pressure. The residue was purified by silica gel column chromatography to provide the desired product (6 g, 73% yield) as a white solid. MS (ESI) m/z=359.0 [M+H]⁺.

### Step 2. Synthesis of 4-((2-aminoethyl)amino)-2-methyl-N-(5-methylthiazol-2-yl)benzamide

A solution of 4-iodo-2-methyl-N-(5-methylthiazol-2-yl)benzamide (200 mg, 0.559 mmol), ethane-1,2-diamine (207 mg, 2.80 mmol), L-proline (64 mg, 0.559 mmol), CuI (106 mg, 0.559 mmol) and K₂CO₃ (155 mg, 1.12 mmol) in DMF (5 mL) were stirred at 100° C. for 1 h by microwave irradiation under argon atmosphere. After cooling down to rt, the mixture was purified by reverse-phase chromatography to provide the desired product (175 mg, 77% yield) as a yellow solid. ¹HNMR (400 MHz, DMSO-d₆) δ 11.88 (brs, 1H), 7.75 (brs, 3H), 7.49 (d, J=9.2 Hz, 1H), 7.15 (s, 1H), 6.48-6.46 (m, 2H), 3.33 (t, J=6.4 Hz, 2H), 2.98-2.93 (m, 2H), 2.39 (s, 3H), 2.35 (s, 3H). MS (ESI) m/z=291.1 [M+H]⁺.

### Example 163. 4-((3-Aminopropyl)amino)-2-methyl-N-(5-methylthiazol-2-yl)benzamide (BL1-184)

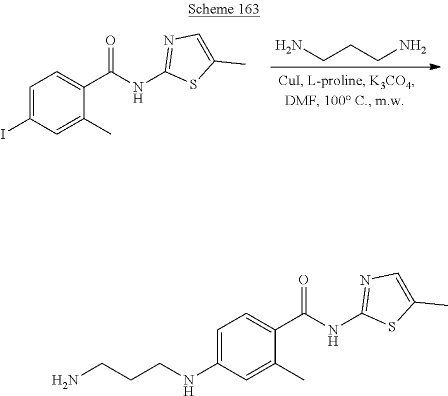 [SMILES: Cc1cnc(NC(=O)c2ccc(I)cc2C)s1 ; NCCCN ; Cc1cnc(NC(=O)c2ccc(NCCCN)cc2C)s1]

BL1-184 was synthesized following the standard procedure for preparing BL1-81 (140 mg, 60% yield) as a white solid. ¹HNMR (400 MHz, DMSO-d₆) δ 11.82 (brs, 1H), 7.82 (brs, 3H), 7.47 (d, J=8.0 Hz, 1H), 7.18 (brs, 1H), 6.44-6.42 (m, 2H), 3.15 (t, J=6.8 Hz, 2H), 2.91-2.86 (m, 2H), 2.38 (s, 3H), 2.35 (s, 3H), 1.84-1.77 (m, 2H). MS (ESI) m/z=305.1 [M+H]⁺.

### Example 164. 4-((4-Aminobutyl)amino)-2-methyl-N-(5-methylthiazol-2-yl)benzamide (BL1-185)

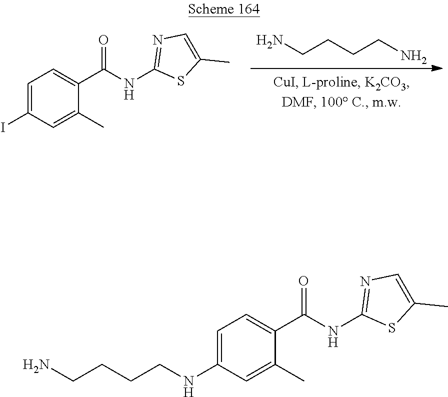 [SMILES: Cc1cnc(NC(=O)c2ccc(I)cc2C)s1 ; NCCCCN ; Cc1cnc(NC(=O)c2ccc(NCCCCN)cc2C)s1]

BL1-185 was synthesized following the standard procedure for preparing BL1-81 (160 mg, 66% yield) as a white solid. ¹HNMR (400 MHz, DMSO-d₆) δ 11.80 (brs, 1H), 7.67 (brs, 3H), 7.45 (d, J=8.4 Hz, 1H), 7.14 (s, 1H), 6.43-6.40 (m, 2H), 3.09 (t, J=6.4 Hz, 1H), 2.84-2.79 (m, 2H), 2.38 (s, 3H), 2.35 (s, 3H), 1.64-1.55 (m, 4H). MS (ESI) m/z=319.1 [M+H]⁺.

### Example 165. 4-((5-Aminopentyl)amino)-2-methyl-N-(5-methylthiazol-2-yl)benzamide (BL1-186)

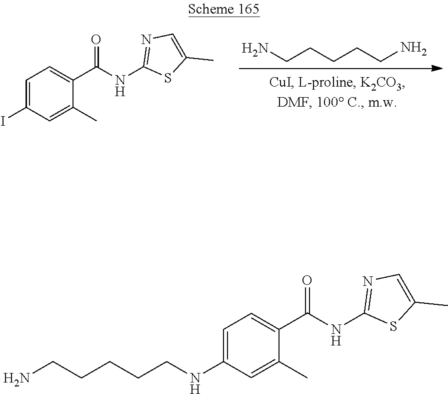 [SMILES: Cc1cnc(NC(=O)c2ccc(I)cc2C)s1 ; NCCCCCN ; Cc1cnc(NC(=O)c2ccc(NCCCCCN)cc2C)s1]

BL1-186 was synthesized following the standard procedure for preparing BL1-81 (120 mg, 61% yield) as a white solid. ¹HNMR (400 MHz, DMSO-d₆) δ 11.83 (brs, 1H), 7.71 (brs, 3H), 7.45 (d, J=8.4 Hz, 1H), 7.15 (s, 1H), 6.42-6.39 (m, 2H), 3.05 (t, J=6.8 Hz, 1H), 2.81-2.74 (m, 2H), 2.37 (s, 3H), 2.34 (s, 3H), 1.60-1.52 (m, 4H), 1.43-1.36 (m, 2H). MS (ESI) m/z=333.1 [M+H]⁺.

### Example 166. 4-((6-Aminohexyl)amino)-2-methyl-N-(5-methylthiazol-2-yl)benzamide (BL1-187)

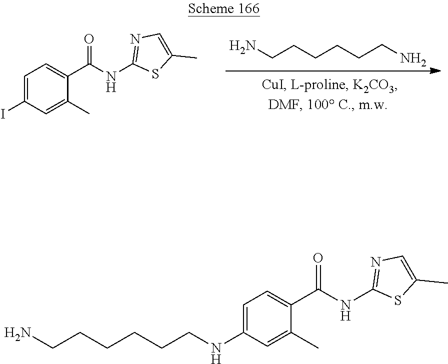 [SMILES: Cc1cnc(NC(=O)c2ccc(I)cc2C)s1 ; NCCCCCCN ; Cc1cnc(NC(=O)c2ccc(NCCCCCCN)cc2C)s1]

BL1-187 was synthesized following the standard procedure for preparing BL1-81 (200 mg, 78% yield) as a white solid. ¹HNMR (400 MHz, DMSO-d₆) δ 11.80 (brs, 1H), 7.66 (brs, 3H), 7.45 (d, J=8.0 Hz, 1H), 7.21 (brs, 1H), 6.42-6.40 (m, 2H), 3.05 (t, J=6.8 Hz, 1H), 2.82-2.73 (m, 2H), 2.37 (s, 3H), 2.35 (s, 3H), 1.57-1.49 (m, 4H), 1.40-1.30 (m, 4H). MS (ESI) m/z=347.1 [M+H]⁺.

### Example 167. 4-((7-Aminoheptyl)amino)-2-methyl-N-(5-methylthiazol-2-yl)benzamide (BL1-188)

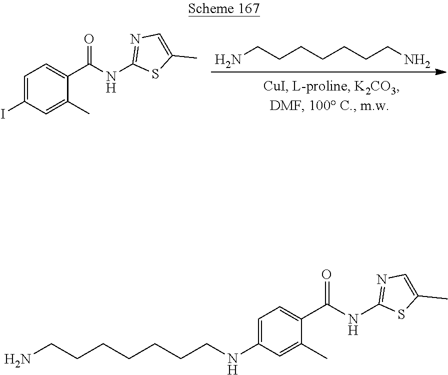 [SMILES: Cc1cnc(NC(=O)c2ccc(I)cc2C)s1 ; NCCCCCCCN ; Cc1cnc(NC(=O)c2ccc(NCCCCCCCN)cc2C)s1]

BL1-188 was synthesized following the standard procedure for preparing BL1-81 (170 mg, 64% yield) as a white solid. ¹HNMR (400 MHz, DMSO-d₆) 11.80 (brs, 1H), 7.69 (brs, 3H), 7.45 (d, J=8.4 Hz, 1H), 7.15 (brs, 1H), 6.42-6.39 (m, 2H), 3.05 (t, J=7.0 Hz, 2H), 2.81-2.73 (m, 2H), 2.37 (s, 3H), 2.34 (s, 3H), 1.54-1.51 (m, 4H), 1.37-1.31 (m, 6H). MS (ESI) m/z=361.2 [M+H]⁺.

### Example 168. 4-((8-Aminooctyl)amino)-2-methyl-N-(5-methylthiazol-2-yl)benzamide (BL1-189)

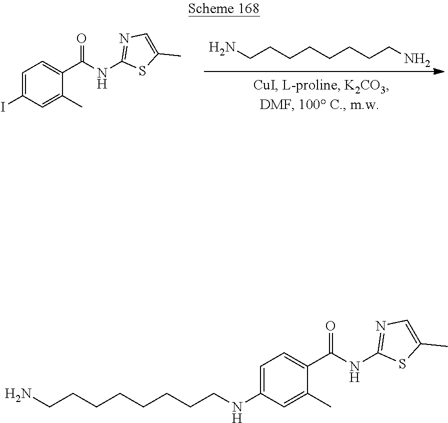 [SMILES: Cc1cnc(NC(=O)c2ccc(I)cc2C)s1 ; NCCCCCCCCN ; Cc1cnc(NC(=O)c2ccc(NCCCCCCCCN)cc2C)s1]

BL1-189 was synthesized following the standard procedure for preparing BL1-81 (160 mg 59% yield) as a white solid. ¹HNMR (400 MHz, DMSO-d₆) δ 11.76 (brs, 1H), 7.63 (brs, 3H), 7.44 (d, J=8.0 Hz, 1H), 7.19 (brs, 1H), 6.41-6.39 (m, 2H), 3.04 (t, J=7.0 Hz, 2H), 2.79-2.73 (m, 2H), 2.37 (s, 3H), 2.35 (s, 3H), 1.57-1.48 (m, 4H), 1.35-1.23 (m, 8H). MS (ESI) m/z=375.2 [M+H]⁺.

### Example 169. 4-((9-Aminononyl)amino)-2-methyl-N-(5-methylthiazol-2-yl)benzamide (BL1-190)

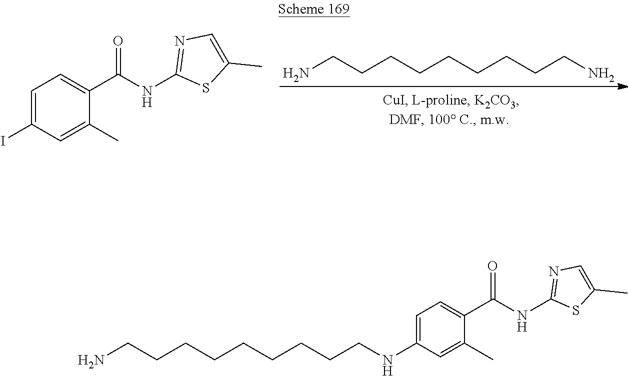

BL1-190 was synthesized following the standard procedure for preparing BL1-81 (70 mg, 25% yield) as a white solid. ¹HNMR (400 MHz, DMSO-d₆) δ 11.86 (brs, 1H), 7.75 (brs, 3H), 7.44 (d, J=8.4 Hz, 1H), 7.15 (brs, 1H), 6.41-6.39 (m, 2H), 3.03 (t, J=7.0 Hz, 2H), 2.94-2.71 (m, 2H), 2.36 (s, 3H), 2.34 (s, 3H), 1.54-1.49 (m, 4H), 1.35-1.26 (m, 10H). MS (ESI) m/z=389.2 [M+H]⁺.

### Example 170. 4-((2-(2-Aminoethoxy)ethyl)amino)-2-methyl-N-(5-methylthiazol-2-yl)benzamide (BL1-191)

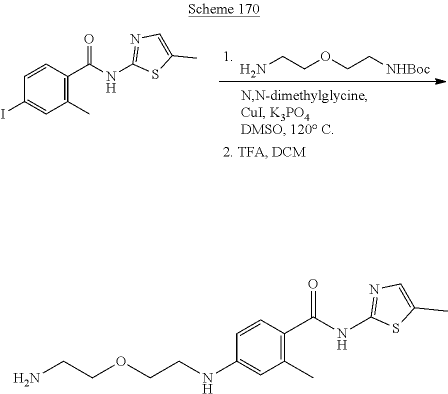 [SMILES: Cc1cnc(NC(=O)c2ccc(I)cc2C)s1 ; CC(C)(C)OC(=O)NCCOCCN ; Cc1cnc(NC(=O)c2ccc(NCCOCCN)cc2C)s1]

### Step 1. Synthesis of tert-butyl (2-(2-((3-methyl-4-((5-methylthiazol-2-yl)carbamoyl)phenyl)amino)ethoxy)ethyl)carbamate

To a solution of 4-iodo-2-methyl-N-(5-methylthiazol-2-yl)benzamide (300 mg, 0.838 mmol) and tert-butyl (2-(2-aminoethoxy)ethyl)carbamate (256 mg, 1.26 mmol) in DMSO (10 mL) were added N,N-dimethylglycine (86 mg, 0.838 mmol), CuI (159 mg, 0.838 mmol) and K₃PO₄ (355 mg, 1.68 mmol) at rt. The reaction mixture was stirred at 120° C. for 4 h under microwave irradiation with argon atmosphere protection. After cooling down to rt, the solution was poured into water (50 mL) and extracted with ethyl acetate (50 mL×3). The combined organic layers were washed with brine, dried over anhydrous Na₂SO₄, filtered and concentrated under reduced pressure. The residue was purified by prep-HPLC to provide the desired product (160 mg, 44% yield) as a yellow solid. MS (ESI) m/z=435.2 [M+H]⁺.

### Step 2. Synthesis of 4-((2-(2-aminoethoxy)ethyl)amino)-2-methyl-N-(5-methylthiazol-2-yl)benzamide

To a solution of tert-butyl (2-(2-((3-methyl-4-((5-methylthiazol-2-yl)carbamoyl)phenyl)amino)ethoxy)ethyl)carbamate (160 mg, 0.369 mmol) in DCM (3 mL) was added TFA (2 mL) at rt. After stirring at rt for 1 h, the reaction mixture was concentrated and purified by prep-HPLC to provide the desired compound (121 mg, 98% yield) as a yellow solid. ¹HNMR (400 MHz, DMSO-d₆) 11.82 (s, 1H), 7.77 (s, 3H), 7.46 (d, J=8.0 Hz, 1H), 7.14 (s, 1H), 6.47-6.44 (m, 2H), 3.62-3.59 (m, 4H), 3.29-3.26 (m, 2H), 3.03-2.99 (m, 2H), 2.38 (s, 3H), 2.35 (s, 3H). MS (ESI) m/z=335.2 [M+H]⁺.

### Example 171. 4-((2-(2-(2-Aminoethoxy)ethoxy)ethyl)amino)-2-methyl-N-(5-methylthiazol-2-yl)benzamide (BL1-192)

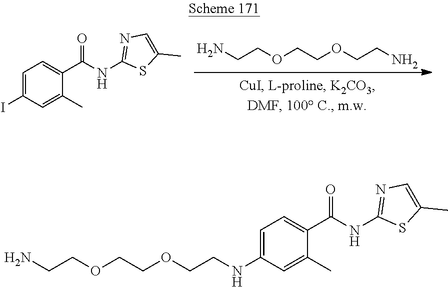 [SMILES: Cc1cnc(NC(=O)c2ccc(I)cc2C)s1 ; NCCOCCOCCN ; Cc1cnc(NC(=O)c2ccc(NCCOCCOCCN)cc2C)s1]

BL1-192 was synthesized following the standard procedure for preparing BL1-81 (110 mg, 40% yield) as a white solid. ¹HNMR (400 MHz, DMSO-d₆) δ 11.85 (brs, 1H), 7.86 (brs, 3H), 7.45 (d, J=8.0 Hz, 1H), 7.16 (brs, 1H), 6.47-6.44 (m, 2H), 3.61-3.55 (m, 8H), 3.26 (t, J=5.8 Hz, 2H), 3.00-2.93 (m, 2H), 2.37 (s, 3H), 2.35 (s, 3H). MS (ESI) m/z=379.1 [M+H]⁺.

### Example 172. 4-((2-(2-(2-(2-Aminoethoxy)ethoxy)ethoxy)ethyl)amino)-2-methyl-N-(5-methylthiazol-2-yl)benzamide (BL1-193)

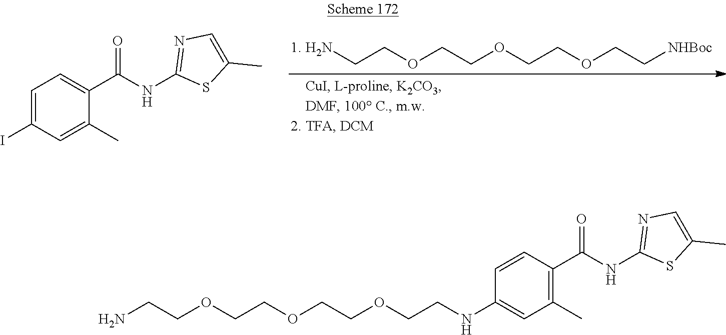

BL1-193 was synthesized following the standard procedure for preparing BL1-81 and BL1-191 (92 mg, 19% yield over 2 steps) as a white solid. ¹HNMR (400 MHz, DMSO-d₆) δ 11.81 (brs, 1H), 7.76 (brs, 3H), 7.45 (d, J=8.0 Hz, 1H), 7.14 (s, 1H), 6.46-6.43 (m, 2H), 3.59-3.55 (m, 12H), 3.25 (t, J=6.4 Hz, 2H), 2.98-2.94 (m, 2H), 2.37 (s, 3H), 2.35 (s, 3H). MS (ESI) m/z=423.2 [M+H]⁺.

### Example 173. 4-((14-Amino-3,6,9,12-tetraoxatetradecyl)amino)-2-methyl-N-(5-methylthiazol-2-yl)benzamide (BL1-194)

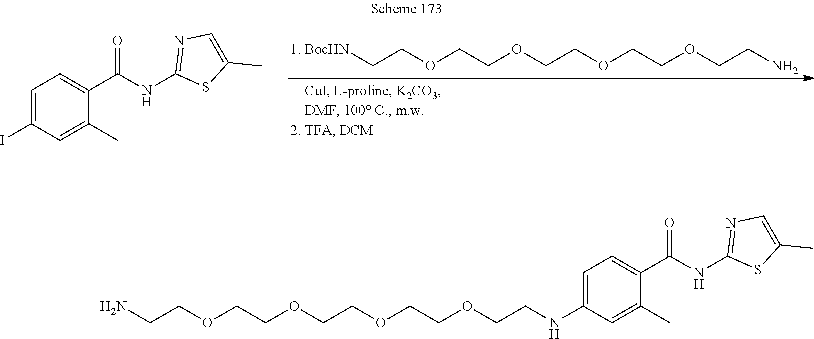

BL1-194 was synthesized following the standard procedure for preparing BL1-81 and BL1-191 (160 mg, 41% yield over 2 steps) as a white solid. ¹HNMR (400 MHz, DMSO-d₆) δ 11.83 (brs, 1H), 7.80 (s, 3H), 7.45 (d, J=8.4 Hz, 1H), 7.15 (s, 1H), 6.46-6.43 (m, 2H), 3.59-3.53 (m, 16H), 3.26-3.23 (m, 2H), 2.98-2.94 (m, 2H), 2.34 (s, 3H), 2.35 (s, 3H). MS (ESI) m/z=467.2 [M+H]⁺.

### Example 174. 4-((17-Amino-3,6,9,12,15-pentaoxaheptadecyl)amino)-2-methyl-N-(5-methylthiazol-2-yl)benzamide (BL1-195)

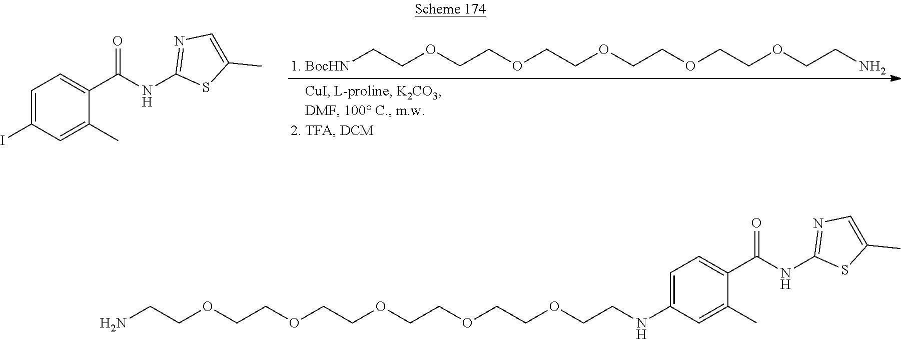

BL1-195 was synthesized following the standard procedure for preparing BL1-81 and BL1-191 (40 mg, 6% yield over 2 steps) as a white solid. ¹HNMR (400 MHz, DMSO-d₆) δ 11.80 (brs, 1H), 7.76 (brs, 3H), 7.47 (d, J=8.4 Hz, 1H), 7.14 (s, 1H), 6.46-6.43 (m, 2H), 3.59-3.51 (m, 20H), 3.25 (t, J=5.6 Hz, 2H), 2.99-2.94 (m, 2H), 2.37 (s, 3H), 2.34 (s, 3H). MS (ESI) m/z=511.2 [M+H]⁺.

### Example 175. 4-((20-Amino-3,6,9,12,15,18-hexaoxaicosyl)amino)-2-methyl-N-(5-methylthiazol-2-yl)benzamide (BL1-196)

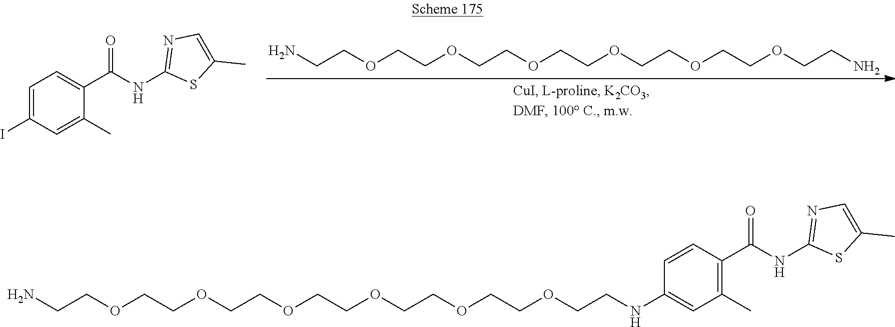 [SMILES: Cc1cnc(NC(=O)c2ccc(I)cc2C)s1 ; NCCOCCOCCOCCOCCOCCOCCN ; Cc1cnc(NC(=O)c2ccc(NCCOCCOCCOCCOCCOCCOCCN)cc2C)s1]

BL1-196 was synthesized following the standard procedure for preparing BL1-81 (85 mg, 17% yield) as a white solid. ¹HNMR (400 MHz, DMSO-d₆) δ 11.79 (brs, 1H), 7.76 (brs, 3H), 7.44 (d, J=8.4 Hz, 1H), 7.15 (brs, 1H), 6.46-6.43 (m, 2H), 3.60-3.50 (m, 20H), 3.25 (t, J=5.6 Hz, 2H), 2.99-2.95 (m, 2H), 2.37 (s, 3H), 2.34 (s, 3H). MS (ESI) m/z=555.3 [M+H]⁺.

### Example 176. 4-((2-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)ethyl)amino)-2-methyl-N-(5-methylthiazol-2-yl)benzamide (CPD-189)

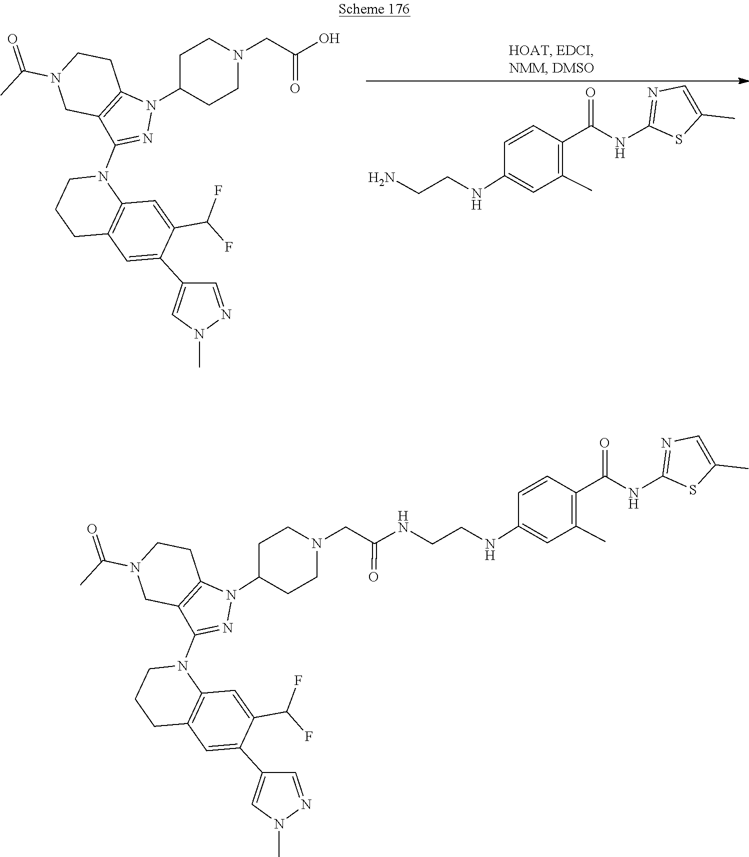 [SMILES: CC(=O)N1CCc2c(c(N3CCCc4cc(-c5cnn(C)c5)c(C(F)F)cc43)nn2C2CCN(CC(=O)O)CC2)C1 ; Cc1cnc(NC(=O)c2ccc(NCCN)cc2C)s1 ; CC(=O)N1CCc2c(c(N3CCCc4cc(-c5cnn(C)c5)c(C(F)F)cc43)nn2C2CCN(CC(=O)NCCNc3ccc(C(=O)Nc4ncc(C)s4)c(C)c3)CC2)C1]

CPD-189 was synthesized following the standard procedure for preparing CPD-008 (4.3 mg, 16% yield). MS (ESI) m/z=840.9 [M+H]⁺.

### Example 177. 4-((3-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)propyl)amino)-2-methyl-N-(5-methylthiazol-2-yl)benzamide (CPD-190)

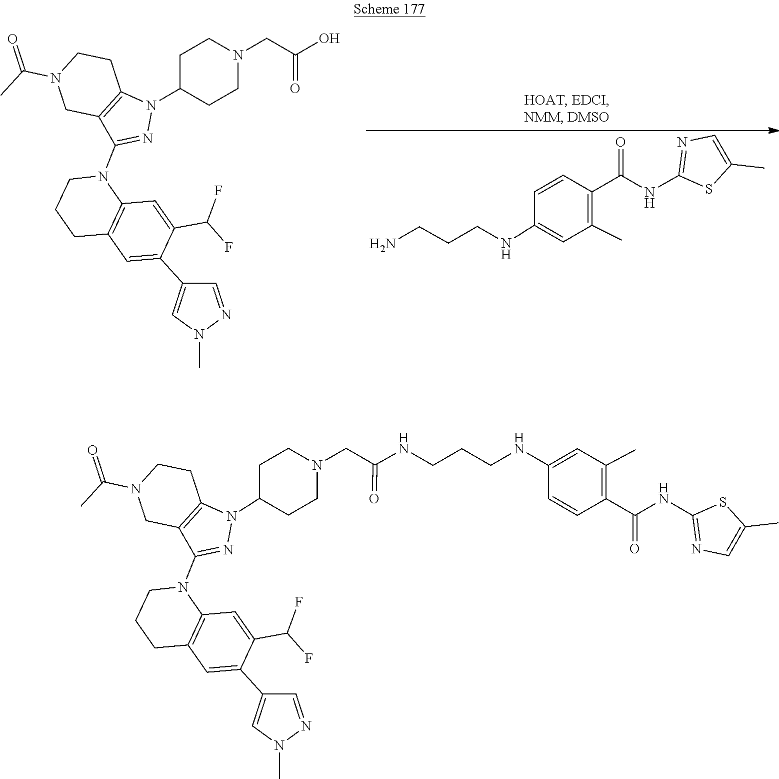 [SMILES: CC(=O)N1CCc2c(c(N3CCCc4cc(-c5cnn(C)c5)c(C(F)F)cc43)nn2C2CCN(CC(=O)O)CC2)C1 ; Cc1cnc(NC(=O)c2ccc(NCCCN)cc2C)s1 ; CC(=O)N1CCc2c(c(N3CCCc4cc(-c5cnn(C)c5)c(C(F)F)cc43)nn2C2CCN(CC(=O)NCCCNc3ccc(C(=O)Nc4ncc(C)s4)c(C)c3)CC2)C1]

CPD-190 was synthesized following the standard procedure for preparing CPD-008 (11.2 mg, 40% yield). MS (ESI) m/z=854.8 [M+H]⁺.

### Example 178. 4-((4-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-d]pyridin-1-yl)piperidin-1-yl)acetamido)butyl)amino)-2-methyl-N-(5-methylthiazol-2-yl)benzamide (CPD-191)

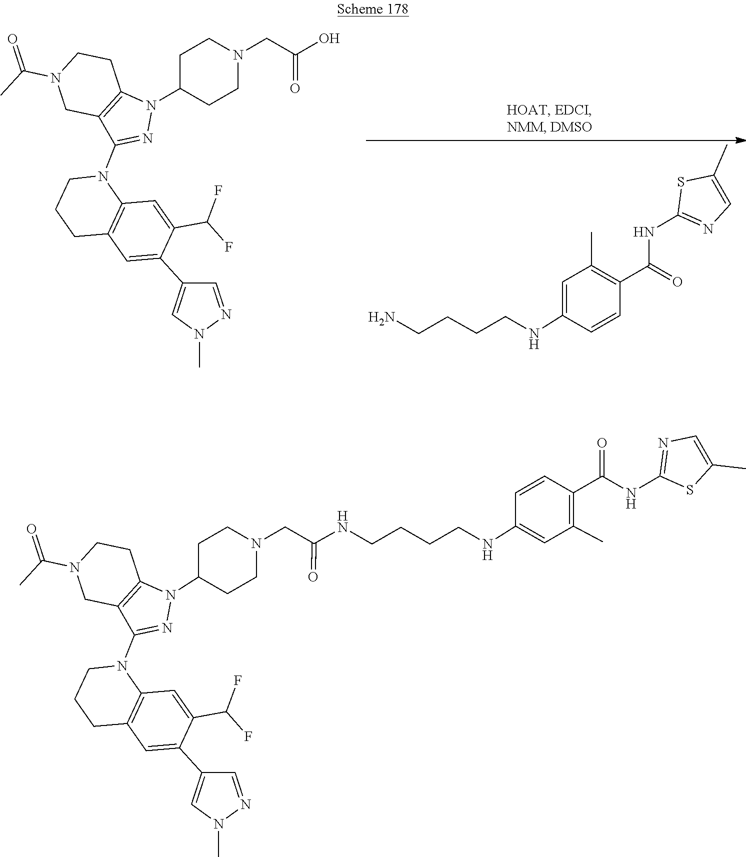 [SMILES: CC(=O)N1CCc2c(c(N3CCCc4cc(-c5cnn(C)c5)c(C(F)F)cc43)nn2C2CCN(CC(=O)O)CC2)C1 ; Cc1cnc(NC(=O)c2ccc(NCCCCN)cc2C)s1 ; CC(=O)N1CCc2c(c(N3CCCc4cc(-c5cnn(C)c5)c(C(F)F)cc43)nn2C2CCN(CC(=O)NCCCCNc3ccc(C(=O)Nc4ncc(C)s4)c(C)c3)CC2)C1]

CPD-191 was synthesized following the standard procedure for preparing CPD-008 (11.7 mg, 38% yield). MS (ESI) m/z=868.7 [M+H]⁺.

### Example 179. 4-((5-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)pentyl)amino)-2-methyl-N-(5-methylthiazol-2-yl)benzamide (CPD-192)

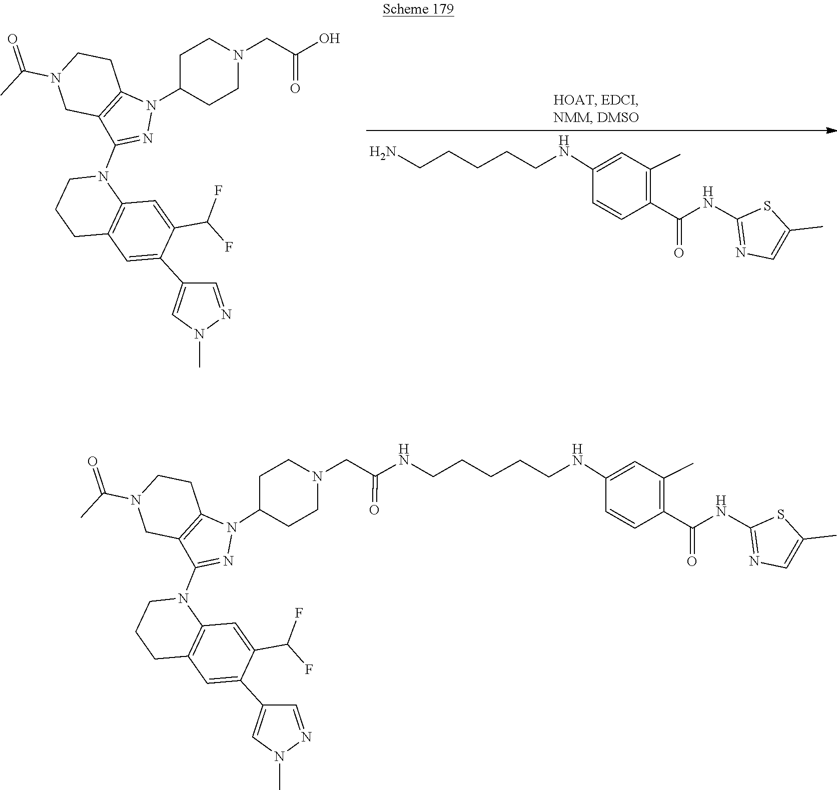 [SMILES: CC(=O)N1CCc2c(c(N3CCCc4cc(-c5cnn(C)c5)c(C(F)F)cc43)nn2C2CCN(CC(=O)O)CC2)C1 ; Cc1cnc(NC(=O)c2ccc(NCCCCCN)cc2C)s1 ; CC(=O)N1CCc2c(c(N3CCCc4cc(-c5cnn(C)c5)c(C(F)F)cc43)nn2C2CCN(CC(=O)NCCCCCNc3ccc(C(=O)Nc4ncc(C)s4)c(C)c3)CC2)C1]

CPD-192 was synthesized following the standard procedure for preparing CPD-008 (10.1 mg, 35% yield). MS (ESI) m/z=882.9 [M+H]⁺.

### Example 180. 4-((6-(2-(4-(A-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)hexyl)amino)-2-methyl-N-(5-methylthiazol-2-yl)benzamide (CPD-193)

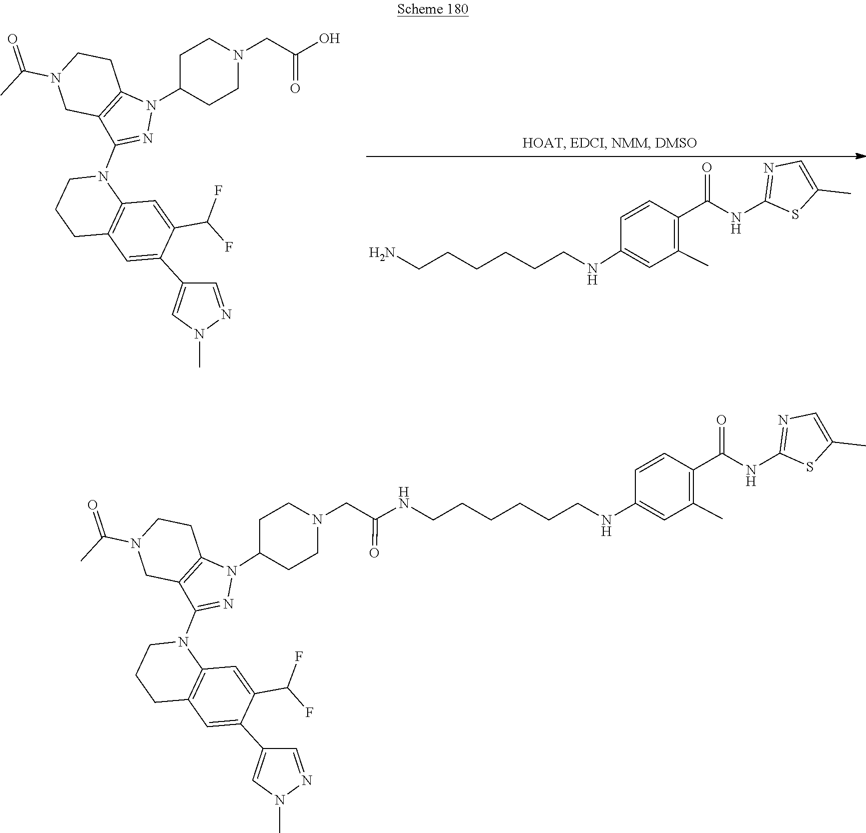 [SMILES: CC(=O)N1CCc2c(c(N3CCCc4cc(-c5cnn(C)c5)c(C(F)F)cc43)nn2C2CCN(CC(=O)O)CC2)C1 ; Cc1cnc(NC(=O)c2ccc(NCCCCCCN)cc2C)s1 ; CC(=O)N1CCc2c(c(N3CCCc4cc(-c5cnn(C)c5)c(C(F)F)cc43)nn2C2CCN(CC(=O)NCCCCCCNc3ccc(C(=O)Nc4ncc(C)s4)c(C)c3)CC2)C1]

CPD-193 was synthesized following the standard procedure for preparing CPD-008 (11.9 mg, 50% yield). MS (ESI) m/z=897.0 [M+H]⁺.

### Example 181. 4-((7-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)heptyl)amino)-2-methyl-N-(5-methylthiazol-2-yl)benzamide (CPD-194)

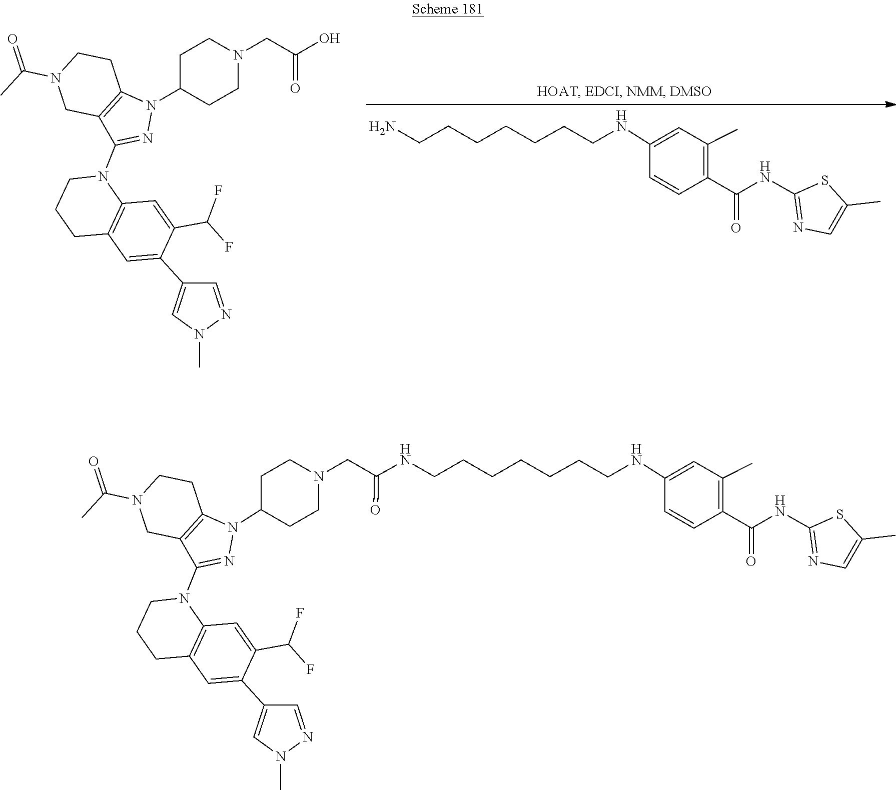 [SMILES: CC(=O)N1CCc2c(c(N3CCCc4cc(-c5cnn(C)c5)c(C(F)F)cc43)nn2C2CCN(CC(=O)O)CC2)C1 ; Cc1cnc(NC(=O)c2ccc(NCCCCCCCN)cc2C)s1 ; CC(=O)N1CCc2c(c(N3CCCc4cc(-c5cnn(C)c5)c(C(F)F)cc43)nn2C2CCN(CC(=O)NCCCCCCCNc3ccc(C(=O)Nc4ncc(C)s4)c(C)c3)CC2)C1]

CPD-194 was synthesized following the standard procedure for preparing CPD-008 (16.5 mg, 51% yield). MS (ESI) m/z=910.9 [M+H]⁺.

### Example 182. 4-((8-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)octyl)amino)-2-methyl-N-(5-methylthiazol-2-yl)benzamide (CPD-195)

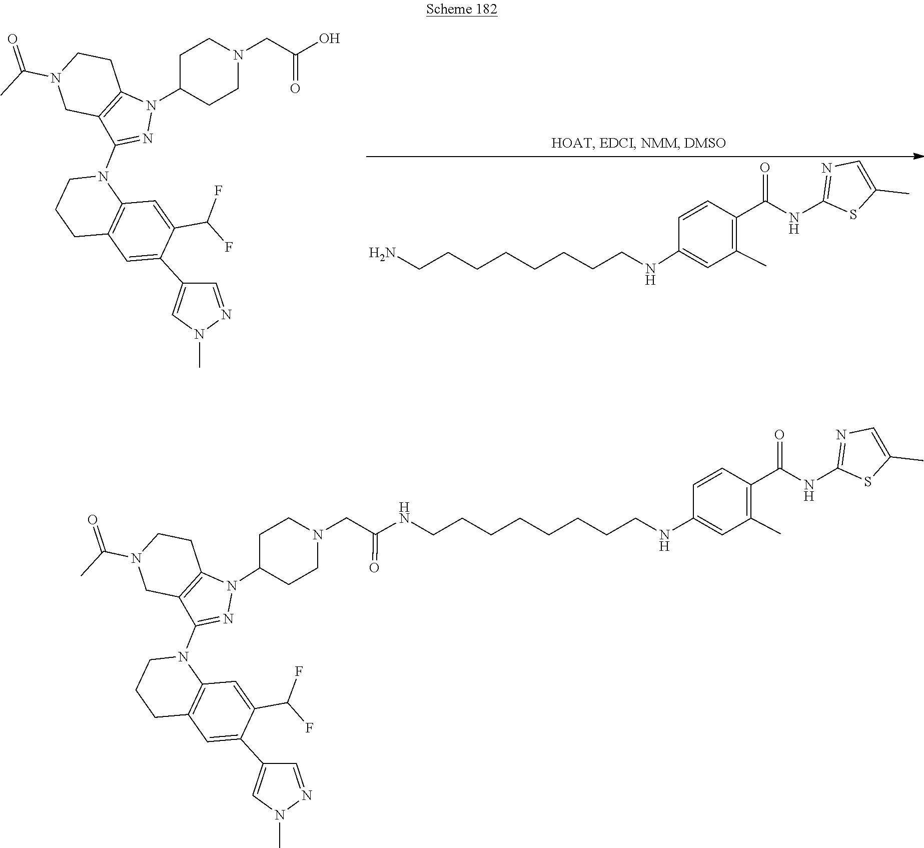 [SMILES: CC(=O)N1CCc2c(c(N3CCCc4cc(-c5cnn(C)c5)c(C(F)F)cc43)nn2C2CCN(CC(=O)O)CC2)C1 ; Cc1cnc(NC(=O)c2ccc(NCCCCCCCCN)cc2C)s1 ; CC(=O)N1CCc2c(c(N3CCCc4cc(-c5cnn(C)c5)c(C(F)F)cc43)nn2C2CCN(CC(=O)NCCCCCCCCNc3ccc(C(=O)Nc4ncc(C)s4)c(C)c3)CC2)C1]

CPD-195 was synthesized following the standard procedure for preparing CPD-008 (16.2 mg, 50% yield). MS (ESI) m/z=924.9 [M+H]⁺.

### Example 183. 4-((9-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)nonyl)amino)-2-methyl-N-(5-methylthiazol-2-yl)benzamide (CPD-196)

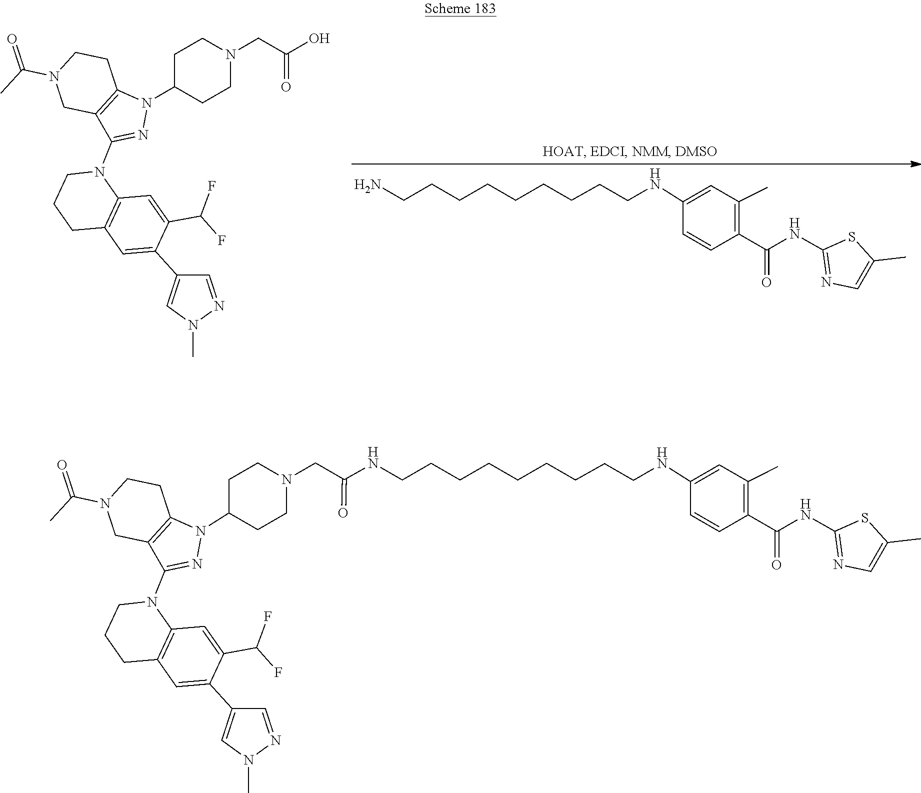 [SMILES: CC(=O)N1CCc2c(c(N3CCCc4cc(-c5cnn(C)c5)c(C(F)F)cc43)nn2C2CCN(CC(=O)O)CC2)C1 ; Cc1cnc(NC(=O)c2ccc(NCCCCCCCCCN)cc2C)s1 ; CC(=O)N1CCc2c(c(N3CCCc4cc(-c5cnn(C)c5)c(C(F)F)cc43)nn2C2CCN(CC(=O)NCCCCCCCCCNc3ccc(C(=O)Nc4ncc(C)s4)c(C)c3)CC2)C1]

CPD-196 was synthesized following the standard procedure for preparing CPD-008 (15.8 mg, 48% yield). MS (ESI) m/z=939.0 [M+H]⁺.

### Example 184. 4-((2-(2-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)ethoxy)ethyl)amino)-2-methyl-N-(5-methylthiazol-2-yl)benzamide (CPD-197)

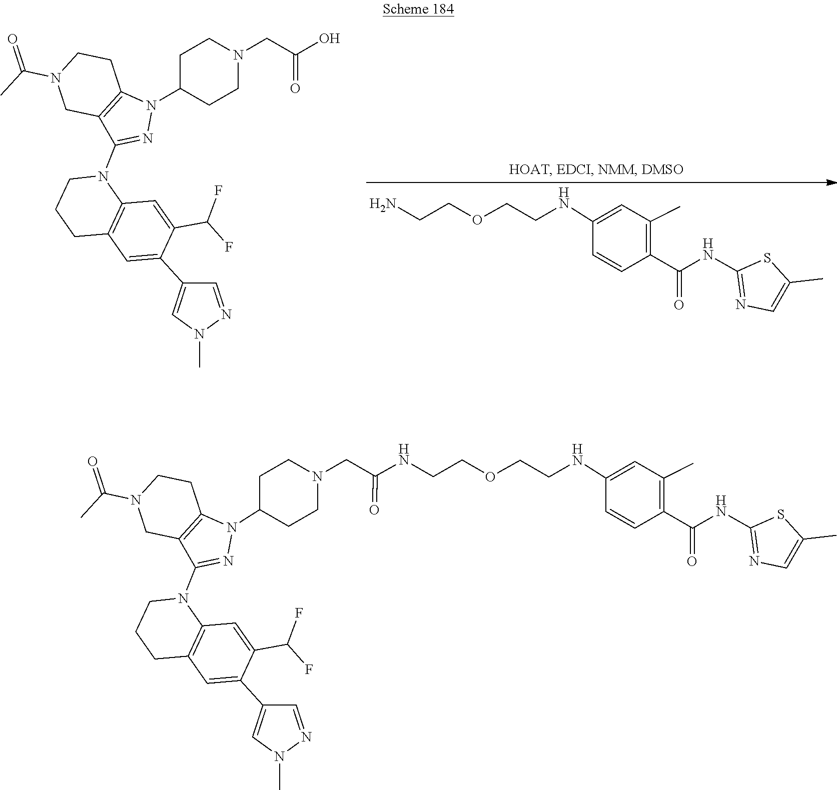 [SMILES: CC(=O)N1CCc2c(c(N3CCCc4cc(-c5cnn(C)c5)c(C(F)F)cc43)nn2C2CCN(CC(=O)O)CC2)C1 ; Cc1cnc(NC(=O)c2ccc(NCCOCCN)cc2C)s1 ; CC(=O)N1CCc2c(c(N3CCCc4cc(-c5cnn(C)c5)c(C(F)F)cc43)nn2C2CCN(CC(=O)NCCOCCNc3ccc(C(=O)Nc4ncc(C)s4)c(C)c3)CC2)C1]

CPD-197 was synthesized following the standard procedure for preparing CPD-008 (23 mg, 65% yield). MS (ESI) m/z=884.7 [M+H]⁺.

### Example 185. 4-((2-(2-(2-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)ethoxy)ethoxy)ethyl)amino)-2-methyl-N-(5-methylthiazol-2-yl)benzamide (CPD-198)

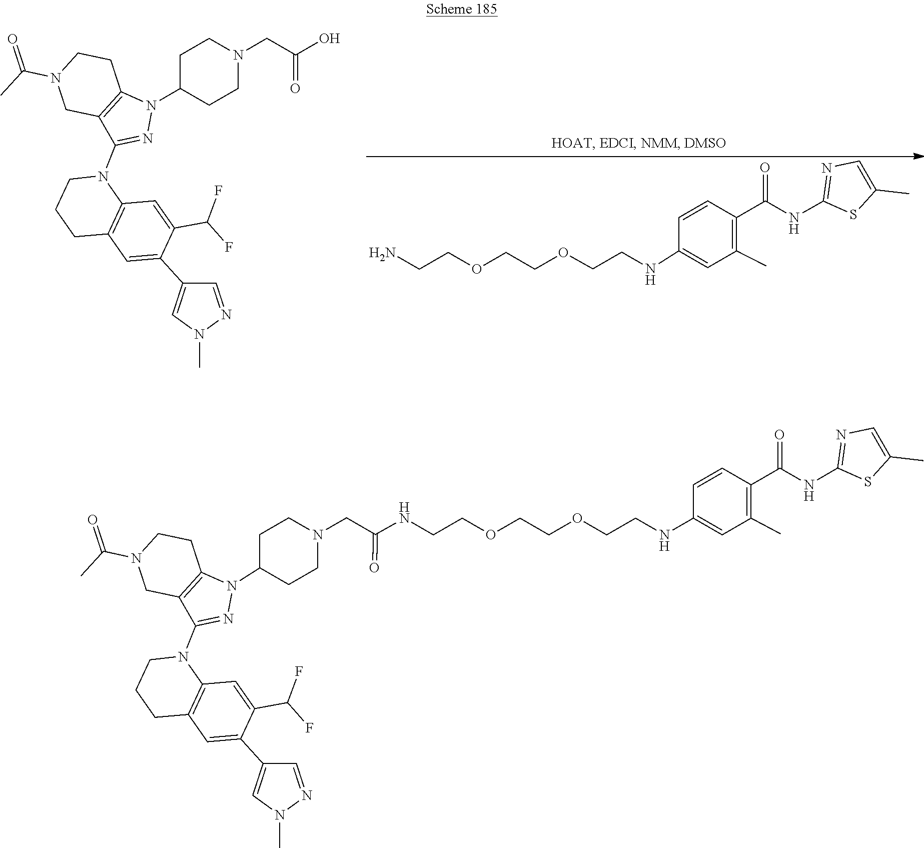 [SMILES: CC(=O)N1CCc2c(c(N3CCCc4cc(-c5cnn(C)c5)c(C(F)F)cc43)nn2C2CCN(CC(=O)O)CC2)C1 ; Cc1cnc(NC(=O)c2ccc(NCCOCCOCCN)cc2C)s1 ; CC(=O)N1CCc2c(c(N3CCCc4cc(-c5cnn(C)c5)c(C(F)F)cc43)nn2C2CCN(CC(=O)NCCOCCOCCNc3ccc(C(=O)Nc4ncc(C)s4)c(C)c3)CC2)C1]

CPD-198 was synthesized following the standard procedure for preparing CPD-008 (18 mg, 49% yield). MS (ESI) m/z=928.8 [M+H]⁺.

### Example 186. 2-(4-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-4-oxobutanamido)-N-(4-methyl-5-nitrothiazol-2-yl)benzamide (CPD-199)

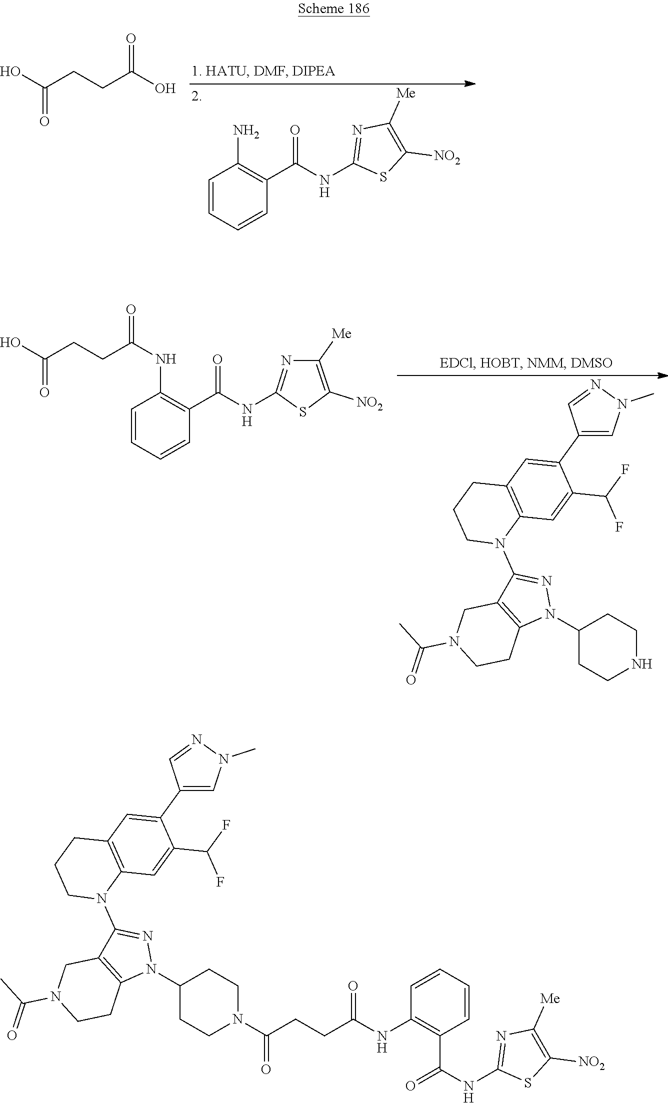 [SMILES: O=C(O)CCC(=O)O ; Cc1nc(NC(=O)c2ccccc2N)sc1[N+](=O)[O-] ; Cc1nc(NC(=O)c2ccccc2NC(=O)CCC(=O)O)sc1[N+](=O)[O-] ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(C(=O)CCC(=O)Nc3ccccc3C(=O)Nc3nc(C)c([N+](=O)[O-])s3)CC2)C1]

### Step 1. Synthesis of 4-((2-((4-methyl-5-nitrothiazol-2-yl)carbamoyl)phenyl)amino)-4-oxobutanoic acid

A solution of succinic acid (700 mg, 5.40 mmol), HATU (615 mg, 1.62 mmol) and DIPEA (418 mg, 3.24 mmol) in DMF (10 mL) was stirred at rt for 30 min. Then 2-amino-N-(4-methyl-5-nitrothiazol-2-yl)benzamide (300 mg, 1.08 mmol) was added at rt. After stirring at rt overnight, the reaction mixture was purified by reverse-phase chromatography to provide the desired product (120 mg, 32% yield) as a white solid. ¹HNMR (400 MHz, DMSO-d₆) δ 13.45 (brs, 1H), 12.24 (brs, 1H), 10.25 (brs, 1H), 7.75-7.71 (m, 2H), 7.57 (dt, J=1.2, 8.0 Hz, 1H), 7.25 (dt, J=1.2, 8.0 Hz, 1H), 2.70 (s, 3H), 2.55-2.51 (m, 2H), 2.49-2.44 (m, 2H). MS (ESI) m/z=379.0 [M+H]⁺.

### Step 2. 2-(4-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-4-oxobutanamido)-N-(4-methyl-5-nitrothiazol-2-yl)benzamide

To a solution of 4-((2-((4-methyl-5-nitrothiazol-2-yl)carbamoyl)phenyl)amino)-4-oxobutanoic acid (6.0 mg, 0.0155 mmol) and 1-(3-(7-(difluoromethyl)-6-(1-methyl-1H-pyrazol-4-yl)-3,4-dihydroquinolin-1(2H)-yl)-1-(piperidin-4-yl)-1,4,6,7-tetrahydro-5H-pyrazolo[4,3-c]pyridin-5-yl)ethan-1-one (8.0 mg, 0.0155 mmol) in DMSO (1 mL) was added EDCI (4.5 mg, 0.023 mmol), HOBT (3.1 mg, 0.023 mmol) and NMM (3.8 mg, 0.047 mmol) at rt. After stirring at rt overnight, the reaction mixture was purified by prep-HPLC to provide the desired product (10 mg, 74% yield) as a white solid. MS (ESI) m/z=870.4 [M+H]⁺.

### Example 187. 2-(5-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-5-oxopentanamido)-N-(4-methyl-5-nitrothiazol-2-yl)benzamide (CPD-200)

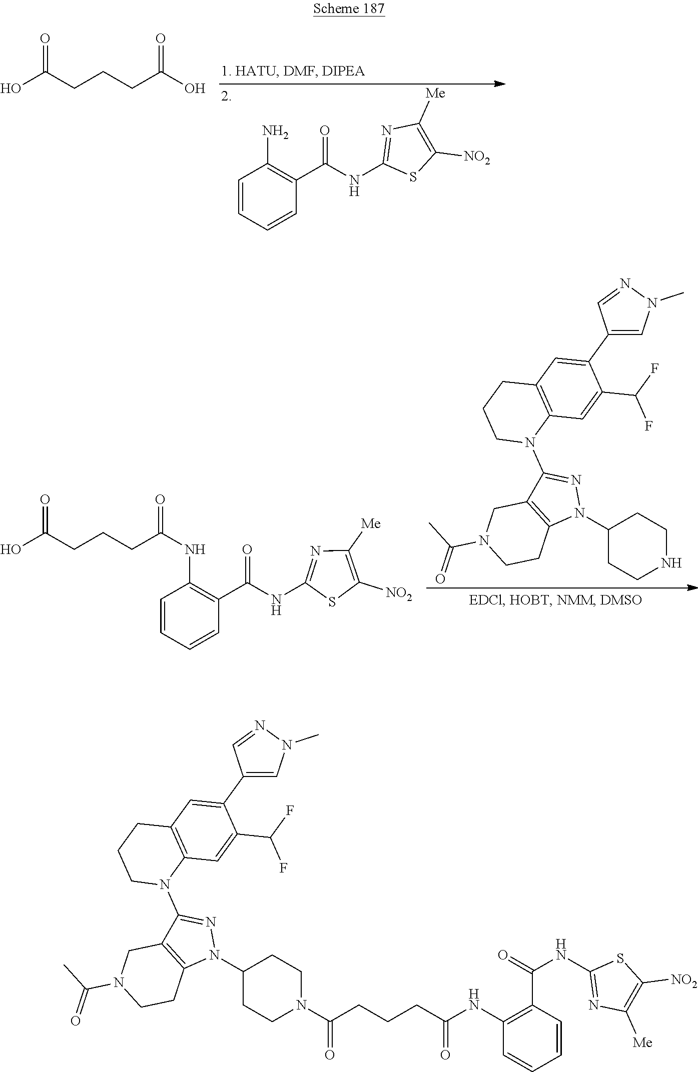 [SMILES: O=C(O)CCCC(=O)O ; Cc1nc(NC(=O)c2ccccc2N)sc1[N+](=O)[O-] ; Cc1nc(NC(=O)c2ccccc2NC(=O)CCCC(=O)O)sc1[N+](=O)[O-] ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(C(=O)CCCC(=O)Nc3ccccc3C(=O)Nc3nc(C)c([N+](=O)[O-])s3)CC2)C1]

CPD-200 was synthesized following the standard procedure for preparing CPD-199 (11.8 mg, 27% yield over 2 steps). MS (ESI) m/z=884.5 [M+H]⁺.

### Example 188. 2-(6-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexanamido)-N-(4-methyl-5-nitrothiazol-2-yl)benzamide (CPD-201)

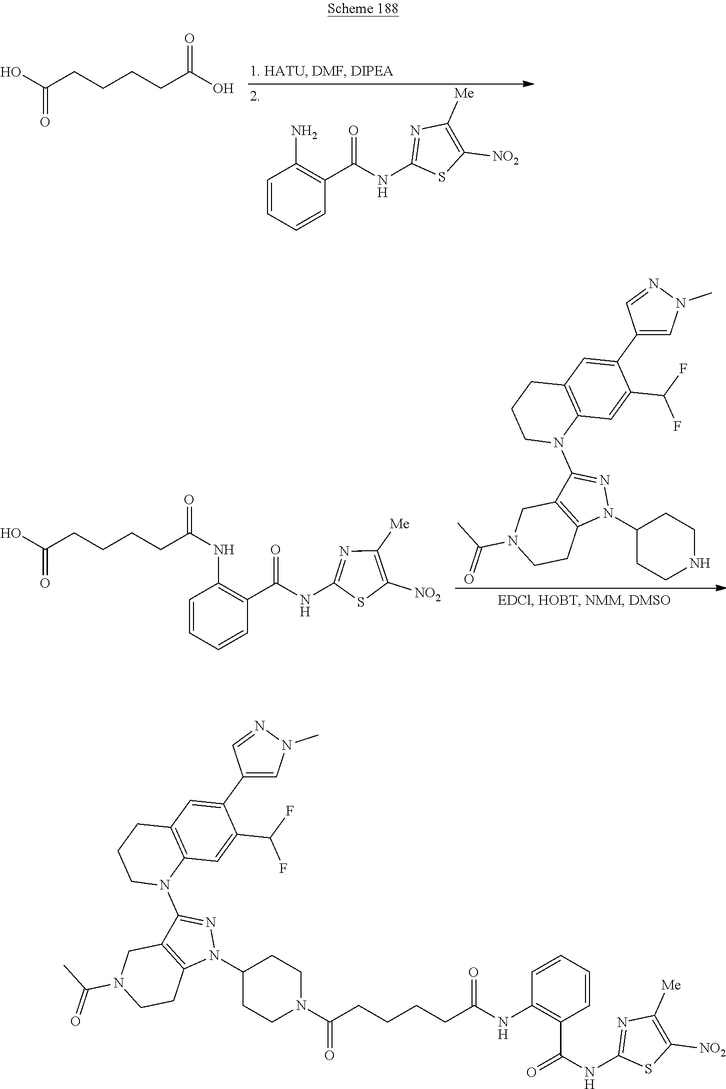 [SMILES: O=C(O)CCCCC(=O)O ; Cc1nc(NC(=O)c2ccccc2N)sc1[N+](=O)[O-] ; Cc1nc(NC(=O)c2ccccc2NC(=O)CCCCC(=O)O)sc1[N+](=O)[O-] ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(C(=O)CCCCC(=O)Nc3ccccc3C(=O)Nc3nc(C)c([N+](=O)[O-])s3)CC2)C1]

CPD-201 was synthesized following the standard procedure for preparing CPD-199 (12.2 mg, 28% yield over 2 steps). MS (ESI) m/z=898.5 [M+H]⁺.

### Example 189. 2-(7-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-7-oxoheptanamido)-N-(4-methyl-5-nitrothiazol-2-yl)benzamide (CPD-202)

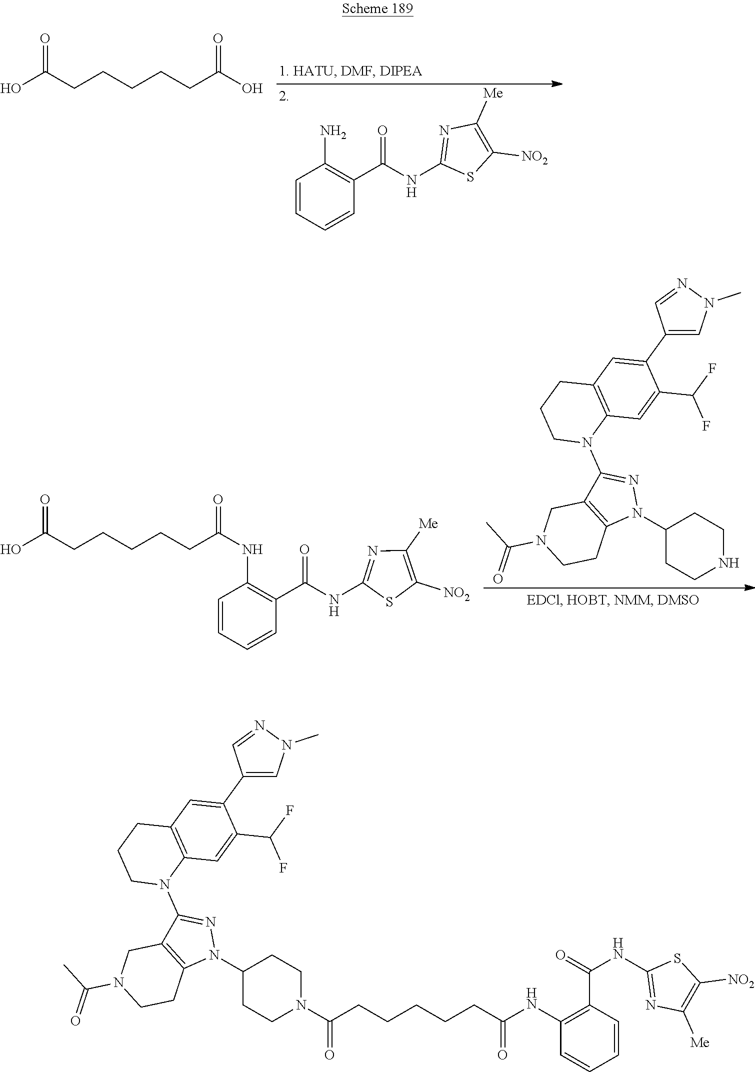 [SMILES: O=C(O)CCCCCC(=O)O ; Cc1nc(NC(=O)c2ccccc2N)sc1[N+](=O)[O-] ; Cc1nc(NC(=O)c2ccccc2NC(=O)CCCCCC(=O)O)sc1[N+](=O)[O-] ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(C(=O)CCCCCC(=O)Nc3ccccc3C(=O)Nc3nc(C)c([N+](=O)[O-])s3)CC2)C1]

CPD-202 was synthesized following the standard procedure for preparing CPD-199 (11.7 mg, 24% yield over 2 steps). MS (ESI) m/z=912.5 [M+H]⁺.

### Example 190. 2-(8-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-8-oxooctanamido)-N-(4-methyl-5-nitrothiazol-2-yl)benzamide (CPD-203)

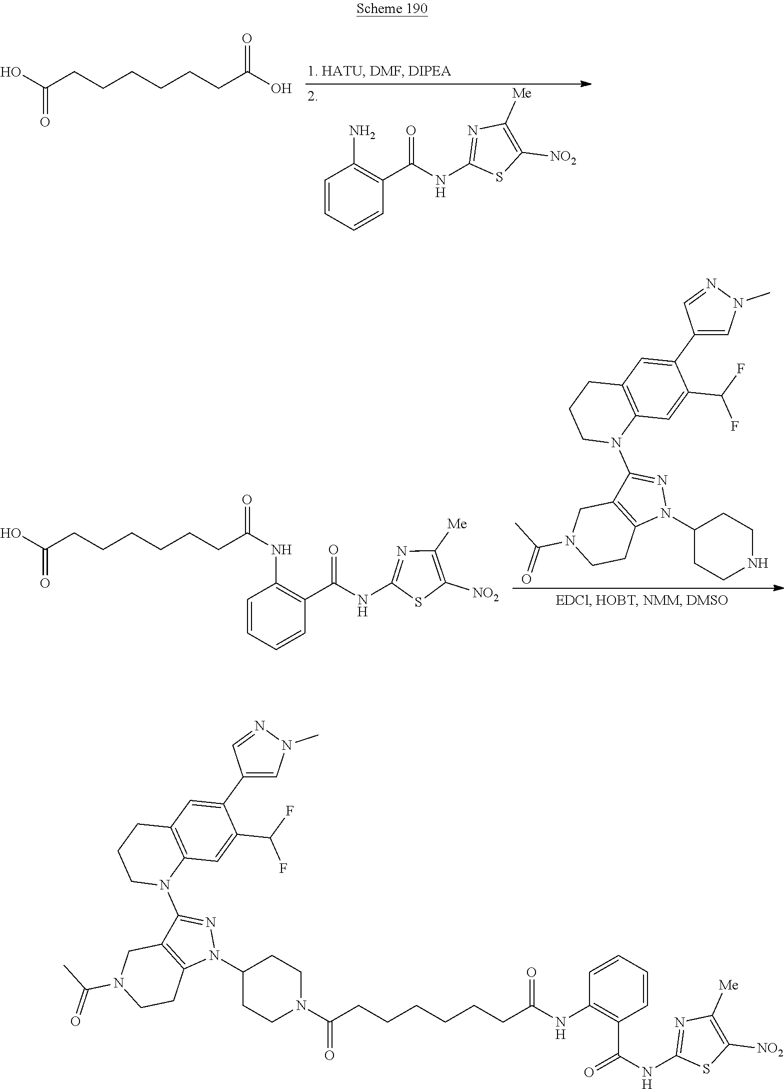 [SMILES: O=C(O)CCCCCCC(=O)O ; Cc1nc(NC(=O)c2ccccc2N)sc1[N+](=O)[O-] ; Cc1nc(NC(=O)c2ccccc2NC(=O)CCCCCCC(=O)O)sc1[N+](=O)[O-] ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(C(=O)CCCCCCC(=O)Nc3ccccc3C(=O)Nc3nc(C)c([N+](=O)[O-])s3)CC2)C1]

CPD-203 was synthesized following the standard procedure for preparing CPD-199 (9.6 mg, 21% yield over 2 steps). MS (ESI) m/z=926.6 [M+H]⁺.

### Example 191. 2-(9-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-9-oxononanamido)-N-(4-methyl-5-nitrothiazol-2-yl)benzamide (CPD-204)

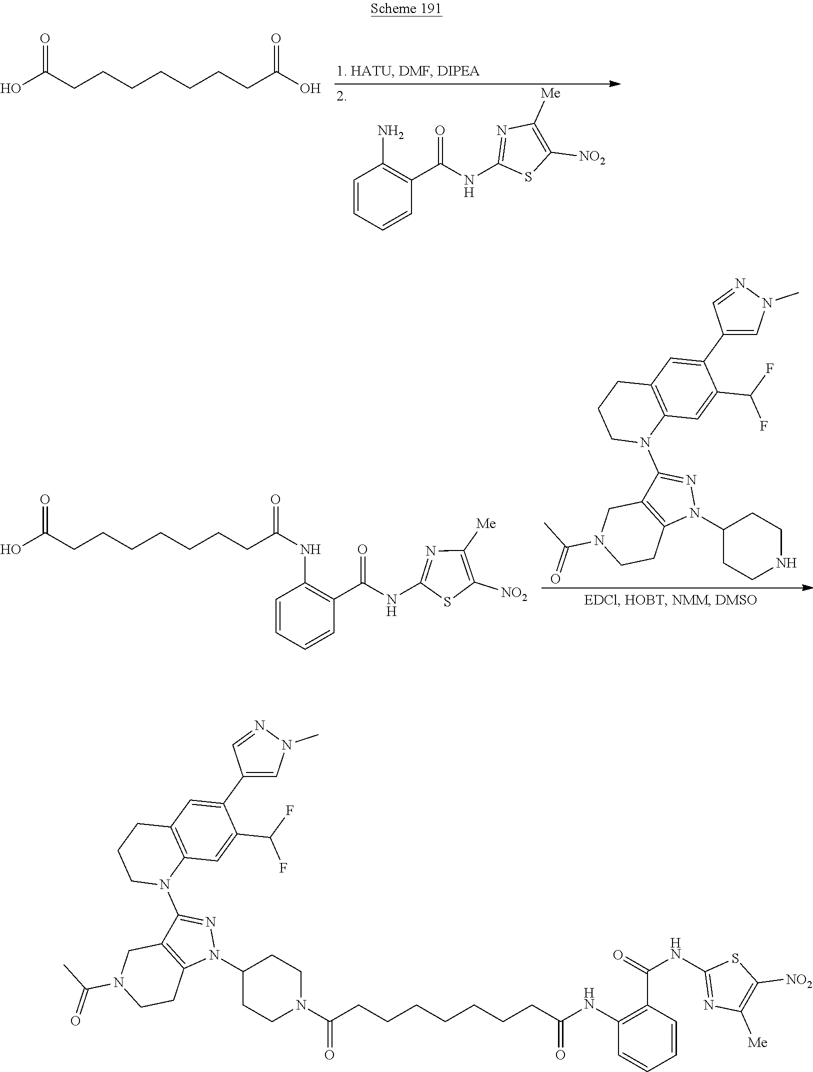 [SMILES: O=C(O)CCCCCCCC(=O)O ; Cc1nc(NC(=O)c2ccccc2N)sc1[N+](=O)[O-] ; Cc1nc(NC(=O)c2ccccc2NC(=O)CCCCCCCC(=O)O)sc1[N+](=O)[O-] ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(C(=O)CCCCCCCC(=O)Nc3ccccc3C(=O)Nc3nc(C)c([N+](=O)[O-])s3)CC2)C1]

CPD-204 was synthesized following the standard procedure for preparing CPD-199 (10.8 mg, 23% yield over 2 steps). MS (ESI) m/z=940.5 [M+H]⁺.

### Example 192. 2-(10-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-10-oxodecanamido)-N-(4-methyl-5-nitrothiazol-2-yl)benzamide (CPD-205)

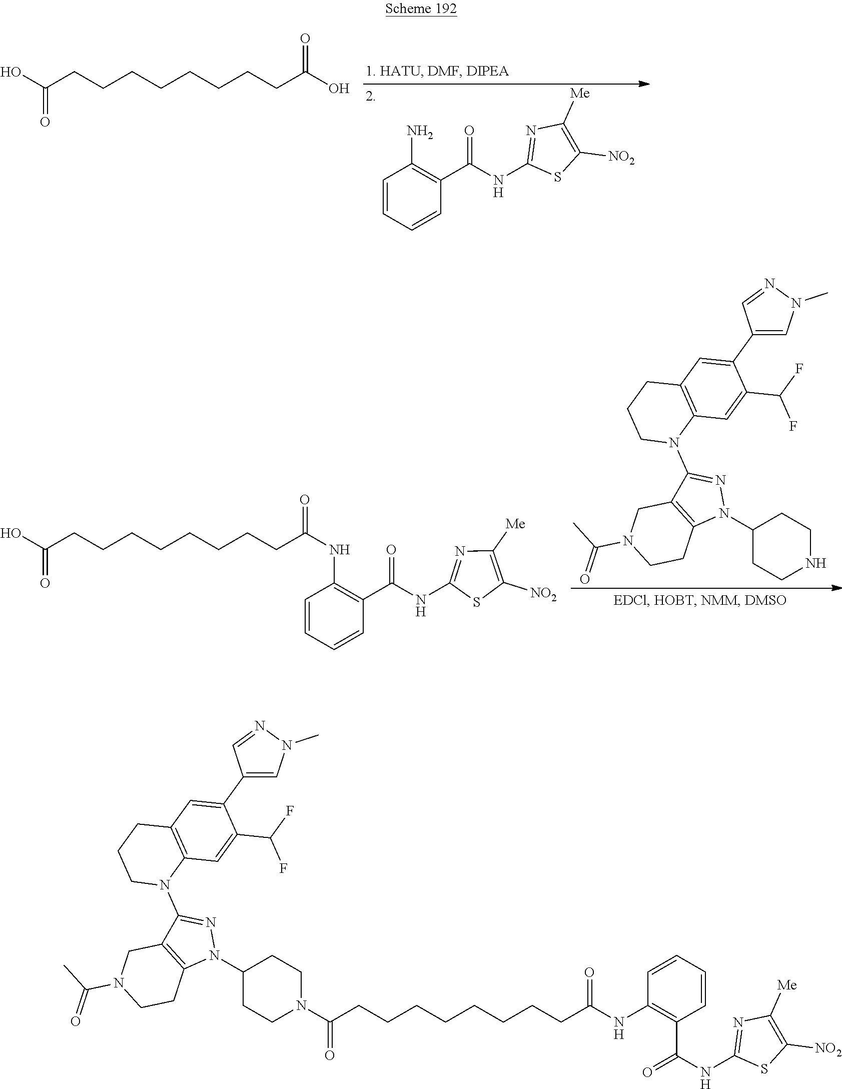 [SMILES: O=C(O)CCCCCCCCC(=O)O ; Cc1nc(NC(=O)c2ccccc2N)sc1[N+](=O)[O-] ; Cc1nc(NC(=O)c2ccccc2NC(=O)CCCCCCCCC(=O)O)sc1[N+](=O)[O-] ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(C(=O)CCCCCCCCC(=O)Nc3ccccc3C(=O)Nc3nc(C)c([N+](=O)[O-])s3)CC2)C1]

CPD-205 was synthesized following the standard procedure for preparing CPD-199 (9.3 mg, 20% yield over 2 steps). MS (ESI) m/z=954.5 [M+H]⁺.

### Example 193. 2-(11-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-11-oxoundecanamido)-N-(4-methyl-5-nitrothiazol-2-yl)benzamide (CPD-206)

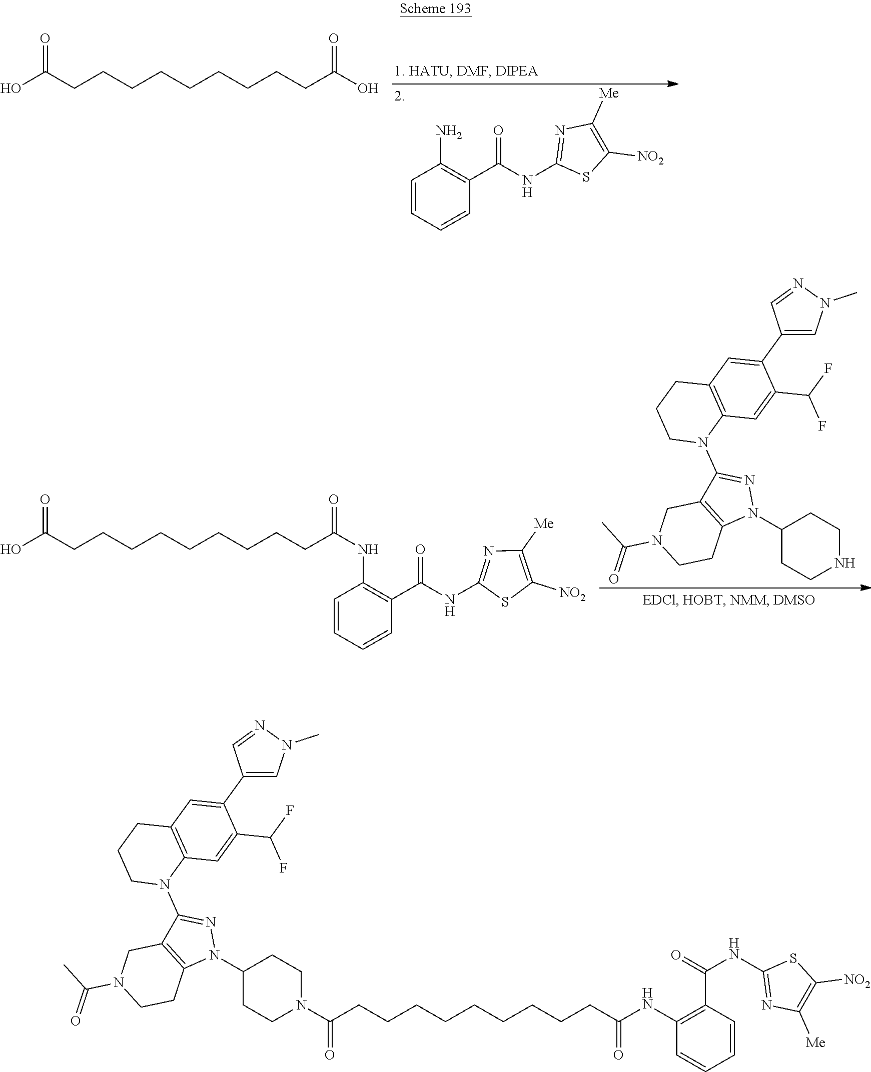 [SMILES: O=C(O)CCCCCCCCCC(=O)O ; Cc1nc(NC(=O)c2ccccc2N)sc1[N+](=O)[O-] ; Cc1nc(NC(=O)c2ccccc2NC(=O)CCCCCCCCCC(=O)O)sc1[N+](=O)[O-] ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(C(=O)CCCCCCCCCC(=O)Nc3ccccc3C(=O)Nc3nc(C)c([N+](=O)[O-])s3)CC2)C1]

CPD-206 was synthesized following the standard procedure for preparing CPD-199 (9.8 mg, 20% yield over 2 steps). MS (ESI) m/z=968.5 [M+H]⁺.

### Example 194. 2-(12-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-12-oxododecanamido)-N-(4-methyl-5-nitrothiazol-2-yl)benzamide (CPD-207)

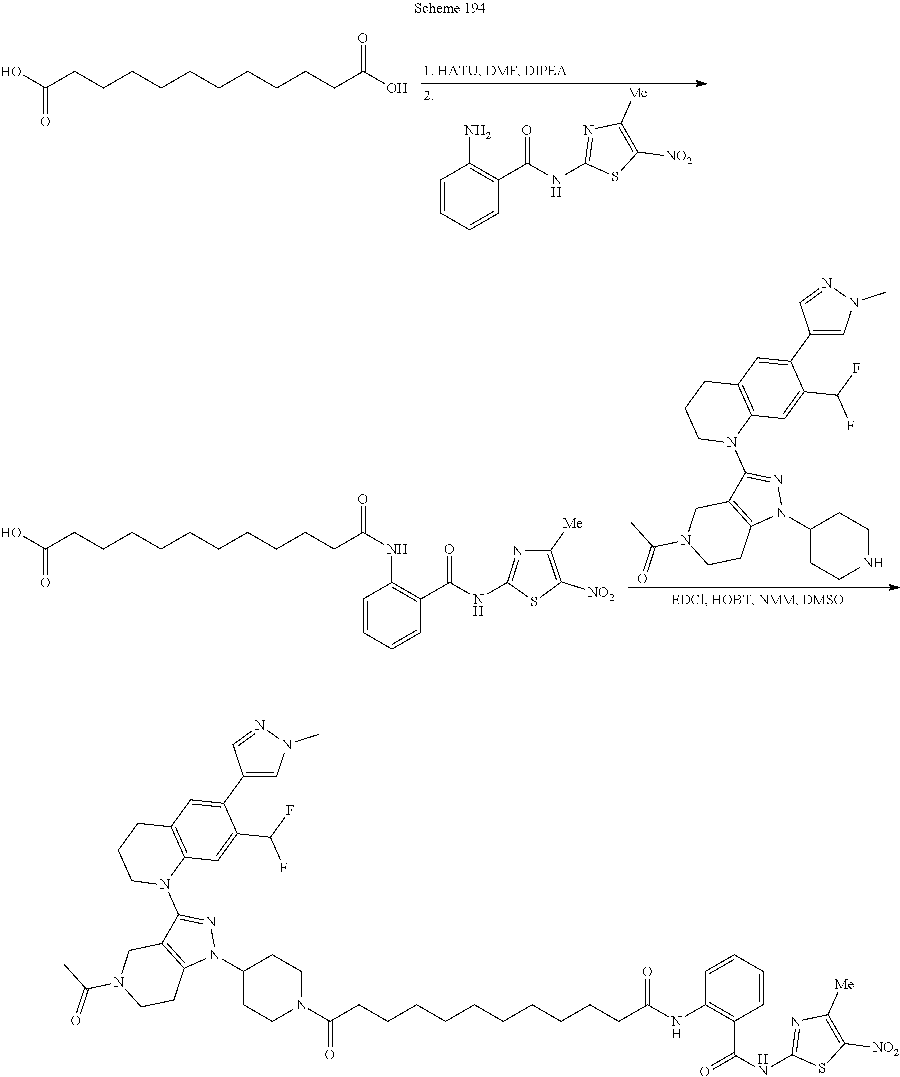 [SMILES: O=C(O)CCCCCCCCCCC(=O)O ; Cc1nc(NC(=O)c2ccccc2N)sc1[N+](=O)[O-] ; Cc1nc(NC(=O)c2ccccc2NC(=O)CCCCCCCCCCC(=O)O)sc1[N+](=O)[O-] ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(C(=O)CCCCCCCCCCC(=O)Nc3ccccc3C(=O)Nc3nc(C)c([N+](=O)[O-])s3)CC2)C1]

CPD-207 was synthesized following the standard procedure for preparing CPD-199 (10.5 mg, 22% yield over 2 steps). MS (ESI) m/z=982.5 [M+H]⁺.

### Example 195. 2-(13-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-13-oxotridecanamido)-N-(4-methyl-5-nitrothiazol-2-yl)benzamide (CPD-208)

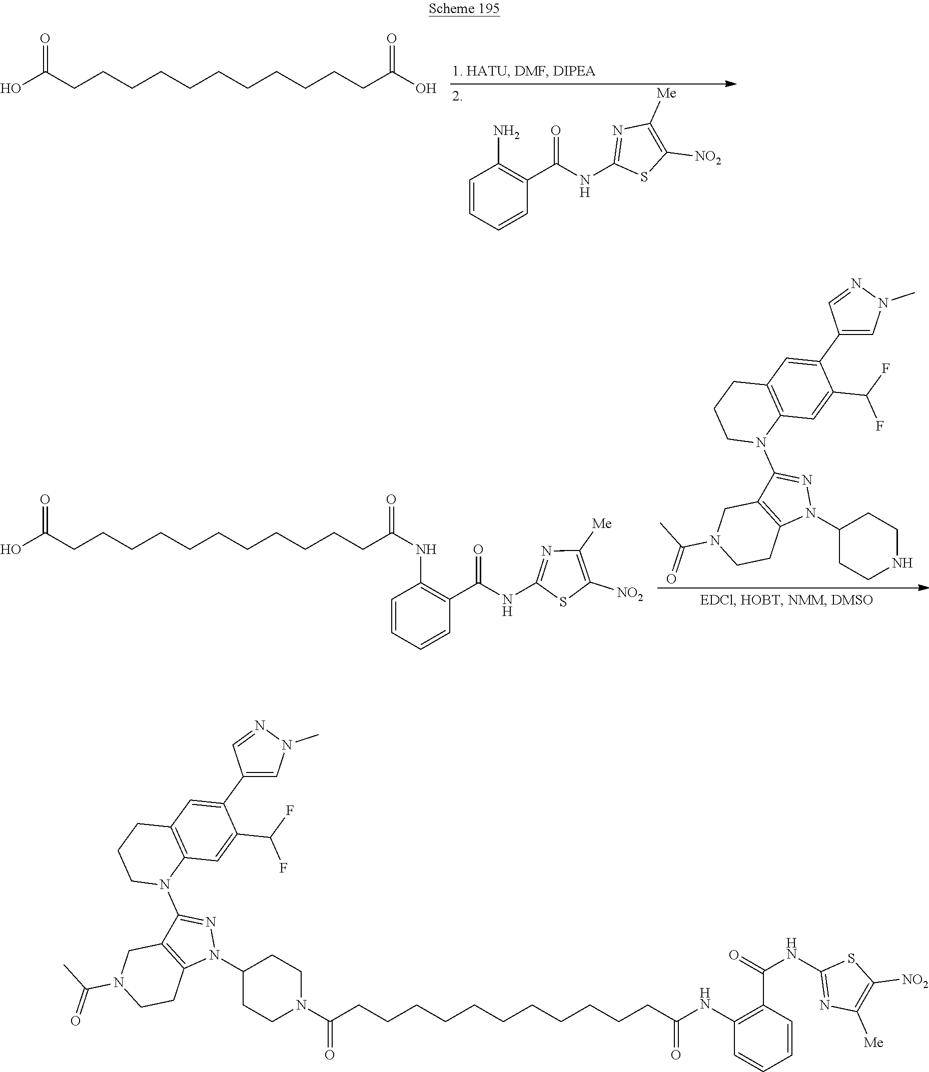 [SMILES: O=C(O)CCCCCCCCCCCC(=O)O ; Cc1nc(NC(=O)c2ccccc2N)sc1[N+](=O)[O-] ; Cc1nc(NC(=O)c2ccccc2NC(=O)CCCCCCCCCCCC(=O)O)sc1[N+](=O)[O-] ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(C(=O)CCCCCCCCCCCC(=O)Nc3ccccc3C(=O)Nc3nc(C)c([N+](=O)[O-])s3)CC2)C1]

CPD-208 was synthesized following the standard procedure for preparing CPD-199 (11.9 mg, 23% yield over 2 steps). MS (ESI) m/z=996.6 [M+H]⁺.

### Example 196. 2-(2-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)acetamido)-N-(4-methyl-5-nitrothiazol-2-yl)benzamide (CPD-209)

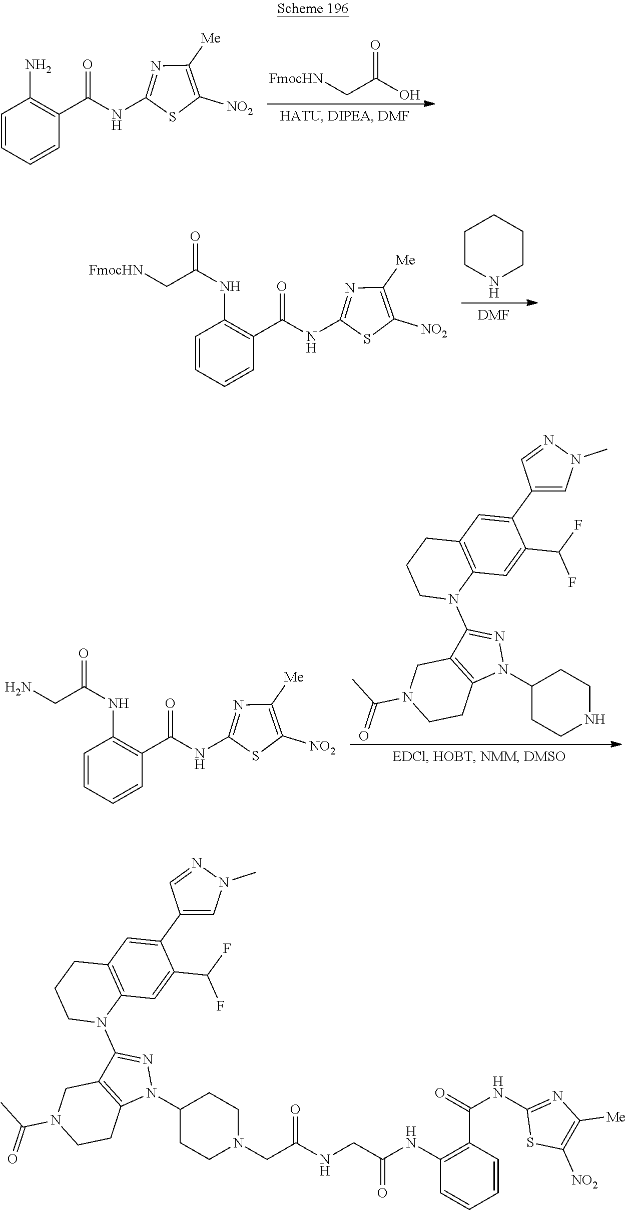

### Step 1. Synthesis of (9H-fluoren-9-yl)methyl (2-((2-((4-methyl-5-nitrothiazol-2-yl)carbamoyl)phenyl)amino)-2-oxoethyl)carbamate

To a solution of (((9H-fluoren-9-yl)methoxy)carbonyl)glycine (427 mg, 1.43 mmol), 2-amino-N-(4-methyl-5-nitrothiazol-2-yl)benzamide (400 mg, 1.43 mmol) in DMF (10 mL) were added HATU (815 mg, 2.15 mmol) and DIPEA (553 mg, 4.29 mmol) at rt. After stirring at rt overnight, the mixture was poured into water (100 mL), acidified to pH=6 by 1N HCl, and extracted with ethyl acetate (50 mL×3). The combined organic layers were washed with brine (100 mL×2), dried over anhydrous Na₂SO₄, filtered and concentrated under reduced pressure to provide the crude desired product (500 mg), which was used directly for next step without further purification. MS (ESI) m/z=558.2 [M+H]⁺.

### Step 2. Synthesis of 2-(2-aminoacetamido)-N-(4-methyl-5-nitrothiazol-2-yl)benzamide

A solution of (9H-fluoren-9-yl)methyl (2-((2-((4-methyl-5-nitrothiazol-2-yl)carbamoyl)phenyl)amino)-2-oxoethyl)carbamate (500 mg, crude) and piperidine (1 mL) in DMF (3 mL) was stirred at rt for 10 min. Then the reaction mixture was purified by prep-HPLC to provide the desired product (100 mg, 21% yield over 2 steps) as a yellow solid. ¹HNMR (400 MHz, DMSO-d₆) δ 14.4 (brs, 1H), 8.67 (brs, 2H), 8.50 (d, J=8.0 Hz, 1H), 8.24 (dd, J=8.0, 1.6 Hz, 1H), 7.50-7.46 (m, 1H), 7.18-7.14 (m, 1H), 3.91 (s, 2H), 2.63 (s, 3H). MS (ESI) m/z=336.1 [M+H]⁺.

### Step 3. Synthesis of 2-(2-(2-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)acetamido)-N-(4-methyl-5-nitrothiazol-2-yl)benzamide

To a solution of 2-(2-aminoacetamido)-N-(4-methyl-5-nitrothiazol-2-yl)benzamide (6.1 mg, 0.018 mmol) and 2-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetic acid (10.3 mg, 0.018 mmol) in DMSO (1 mL) was added EDCI (6.9 mg, 0.036 mmol), HOBT (5.5 mg, 0.036 mmol) and NMM (7.4 mg, 0.09 mmol) at rt. After stirring at rt overnight, the reaction mixture was purified by prep-HPLC to provide the desired product (10 mg, 62% yield) as a white solid. MS (ESI) m/z=885.5 [M+H]⁺.

### Example 197. 2-(3-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)propanamido)-N-(4-methyl-5-nitrothiazol-2-yl)benzamide (CDP-210)

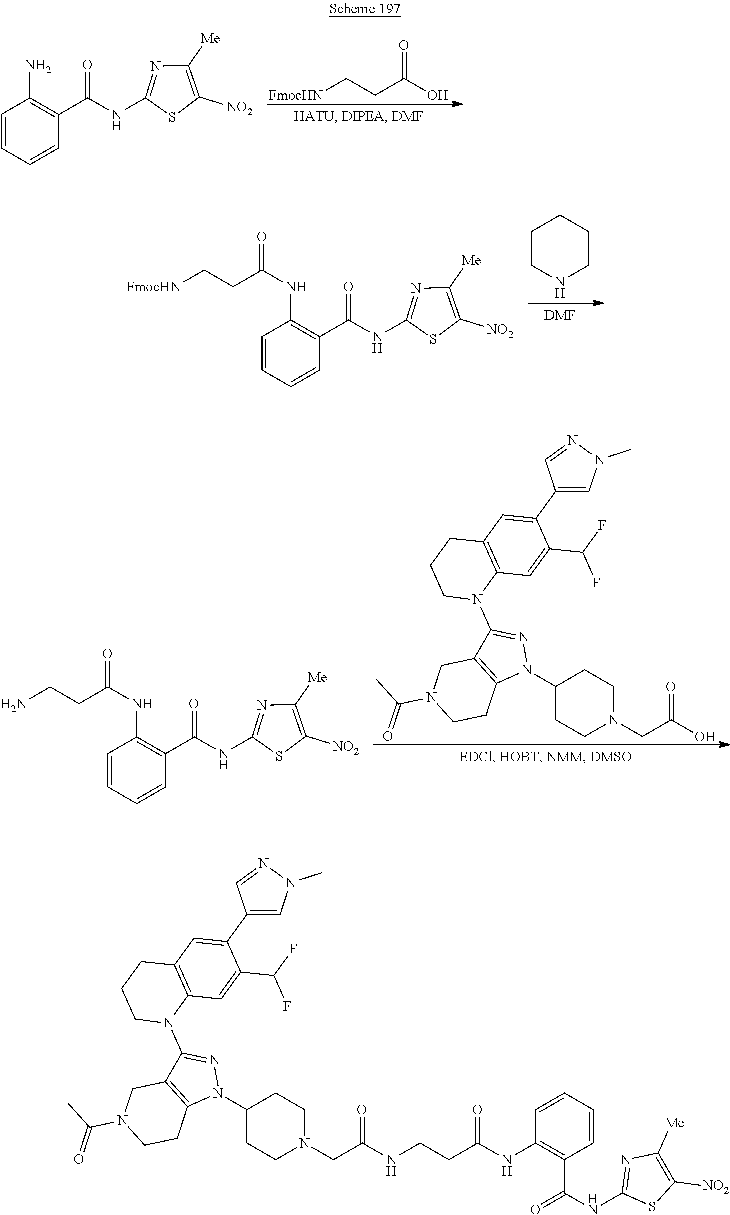

CPD-210 was synthesized following the standard procedure for preparing CPD-209 (6.0 mg, 6% yield over 3 steps). MS (ESI) m/z=899.5 [M+H]⁺.

### Example 198. 2-(4-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)butanamido)-N-(4-methyl-5-nitrothiazol-2-yl)benzamide (CPD-211)

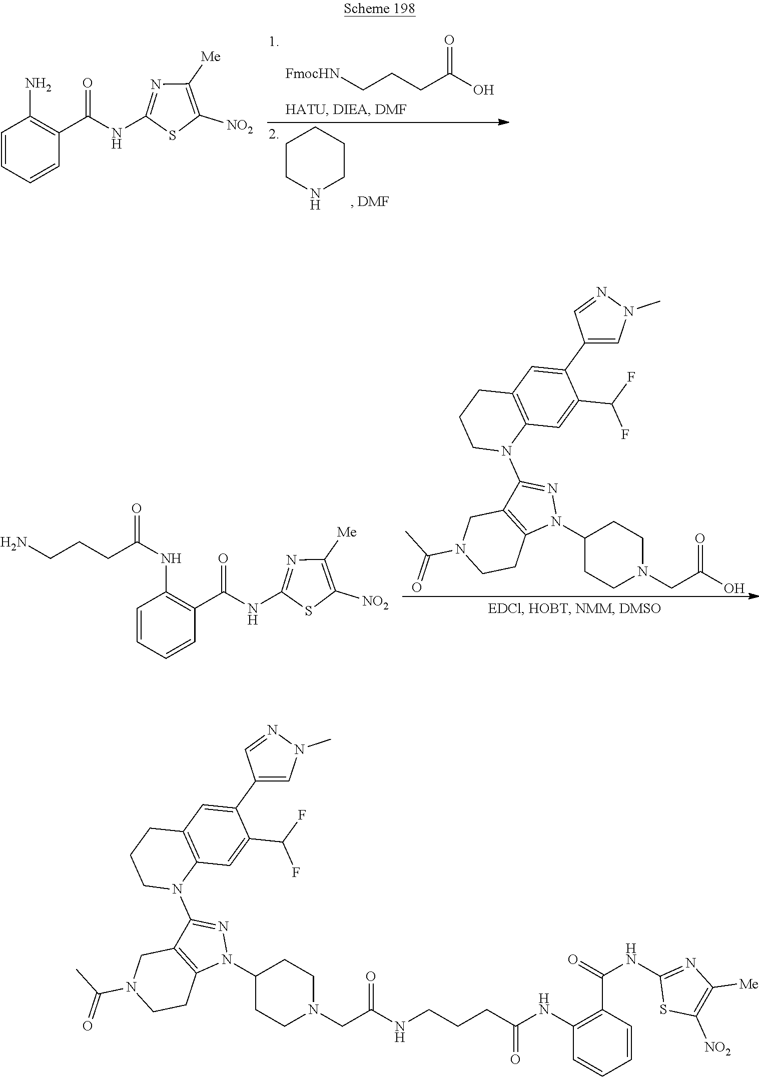

CPD-211 was synthesized following the standard procedure for preparing CPD-209 (11 mg, 28% yield over 3 steps). MS (ESI) m/z=913.5 [M+H]⁺.

### Example 199. 2-(5-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)pentanamido)-N-(4-methyl-5-nitrothiazol-2-yl)benzamide (CPD-212)

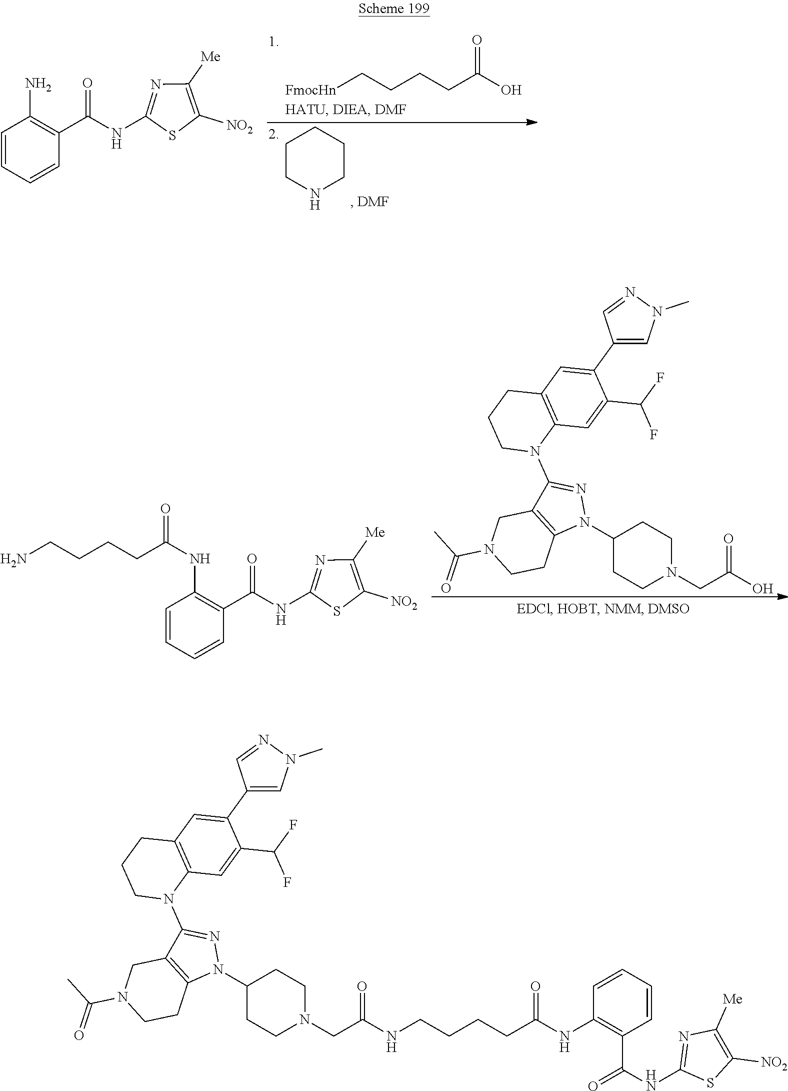

CPD-212 was synthesized following the standard procedure for preparing CPD-209 (9 mg, 4% yield over 3 steps). MS (ESI) m/z=927.6 [M+H]⁺.

### Example 200. 2-(6-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)hexanamido)-N-(4-methyl-5-nitrothiazol-2-yl)benzamide (CDP-213)

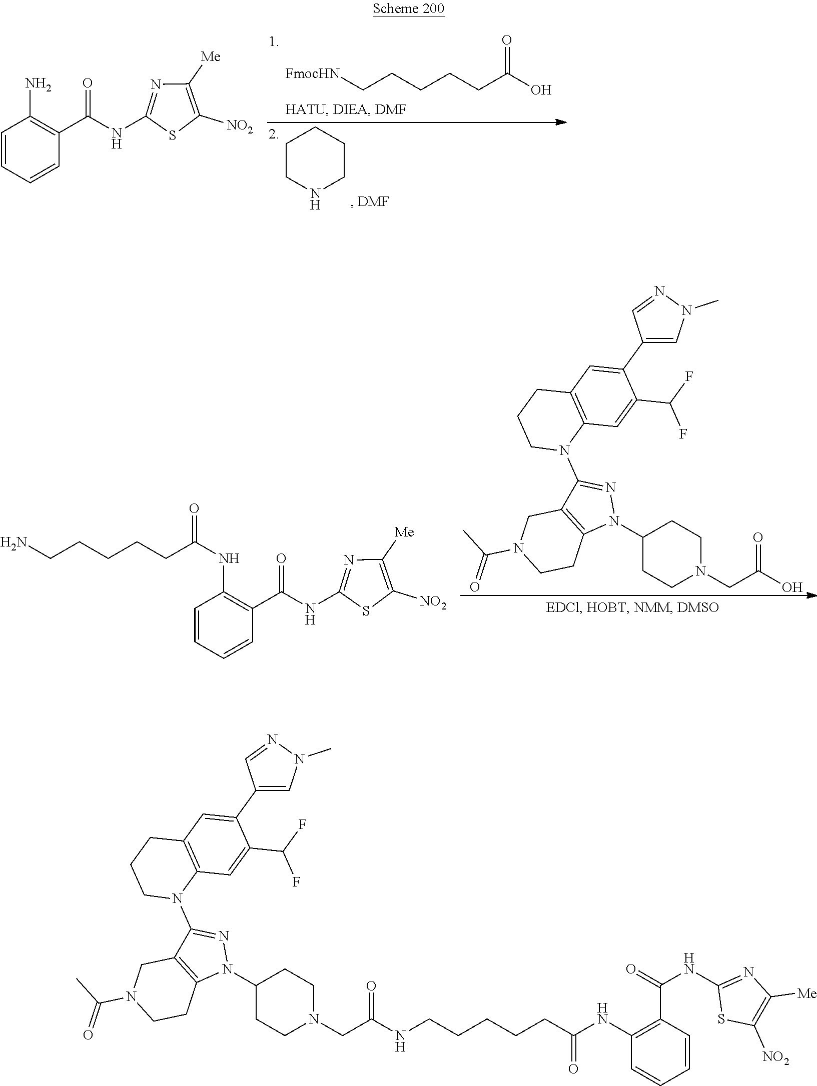

CPD-213 was synthesized following the standard procedure for preparing CPD-209 (11 mg, 68% yield). MS (ESI) m/z=941.6 [M+H]⁺.

### Example 201. 2-(7-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)heptanamido)-N-(4-methyl-5-nitrothiazol-2-yl)benzamide (CPD-214)

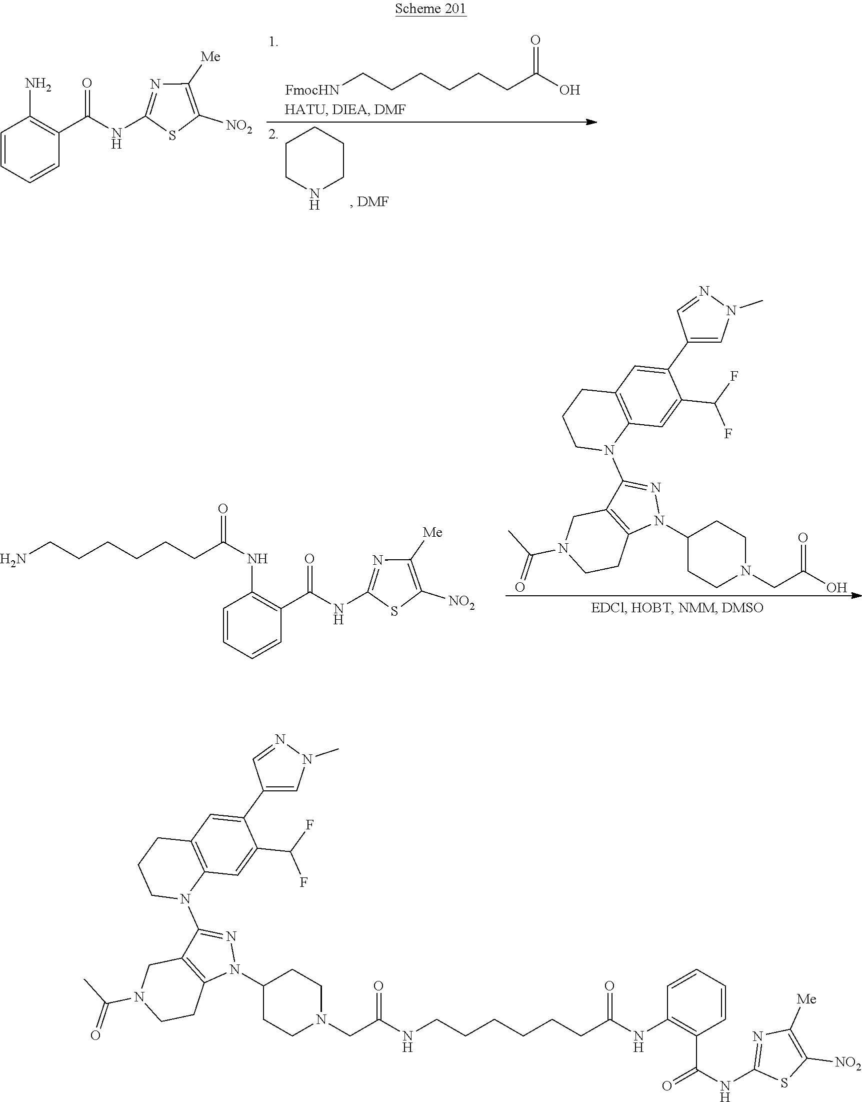

CPD-214 was synthesized following the standard procedure for preparing CPD-209 (12 mg, 70% yield). MS (ESI) m/z=955.6 [M+H]⁺.

### Example 202. 2-(8-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)octanamido)-N-(4-methyl-5-nitrothiazol-2-yl)benzamide (CPD-215)

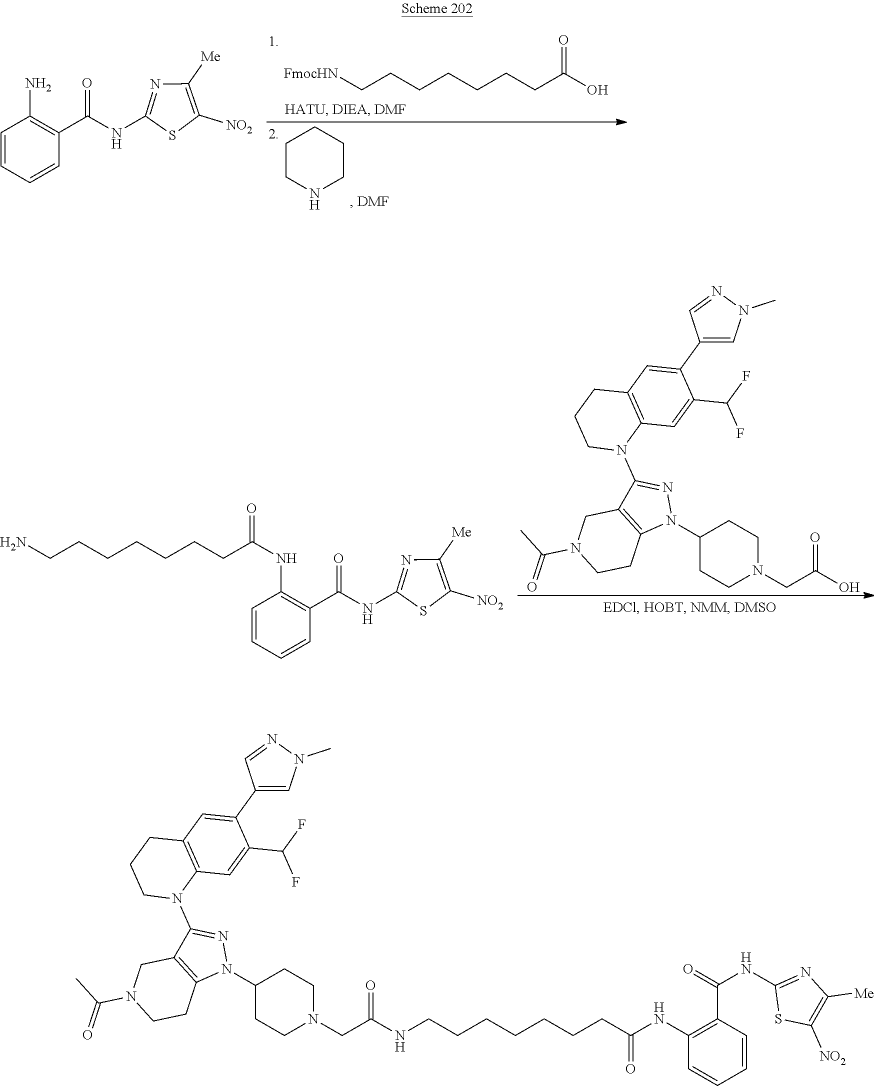

CPD-215 was synthesized following the standard procedure for preparing CPD-209 (15 mg, 85% yield). MS (ESI) m/z=969.6 [M+H]⁺.

### Example 203. 2-(9-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)nonanamido)-N-(4-methyl-5-nitrothiazol-2-yl)benzamide (CPD-216)

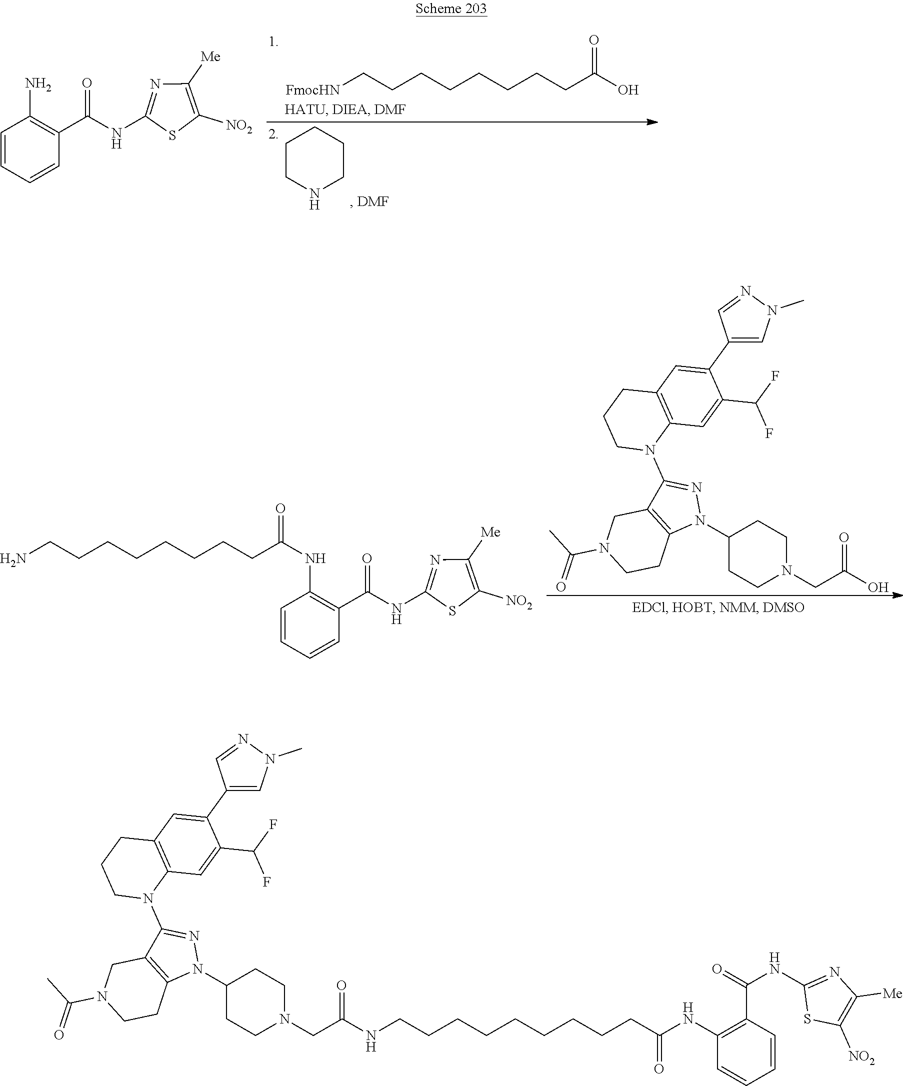

CPD-216 was synthesized following the standard procedure for preparing CPD-209 (13 mg, 77% yield). MS (ESI) m/z=983.6 [M+H]⁺.

### Example 204. 2-(10-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)decanamido)-N-(4-methyl-5-nitrothiazol-2-yl)benzamide (CPD-217)

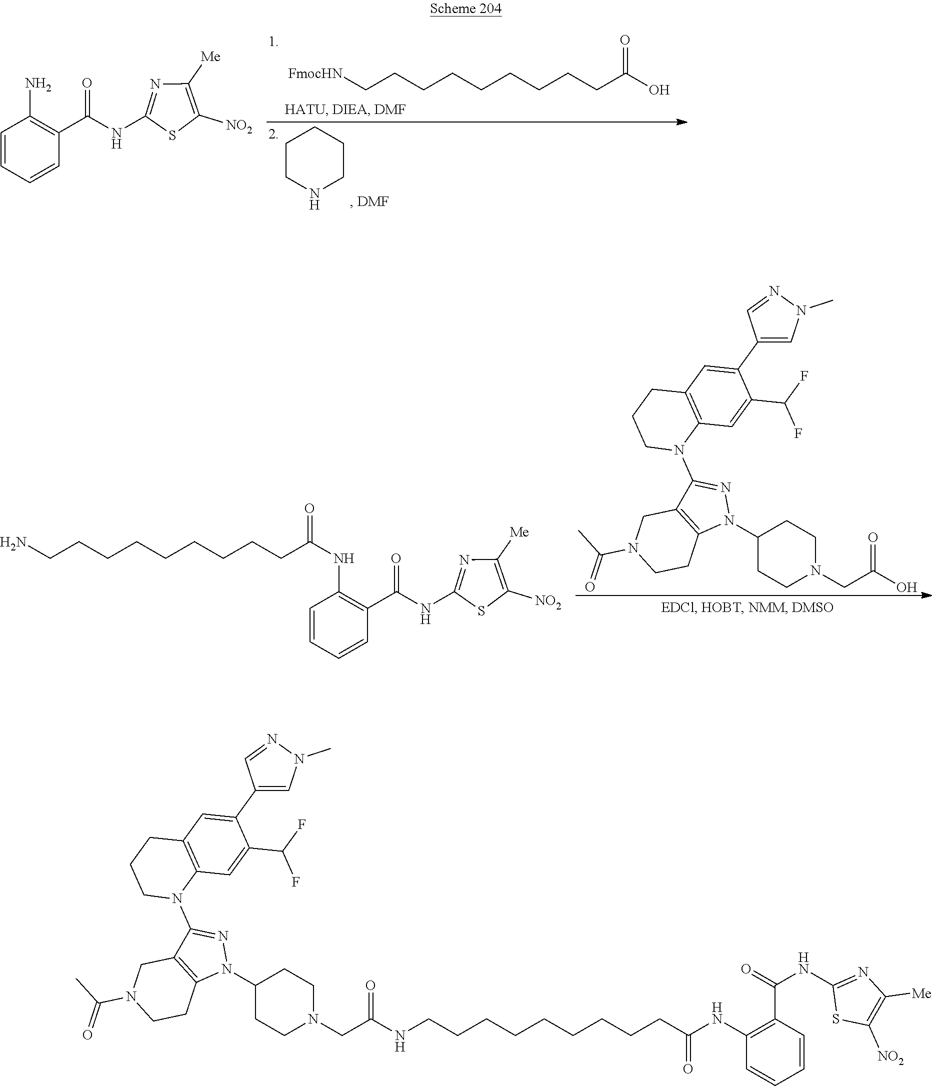

CPD-217 was synthesized following the standard procedure for preparing CPD-209 (10 mg, 57% yield). MS (ESI) m/z=997.6 [M+H]⁺.

### Example 205. 2-(11-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)acetamido)undecanamido)-N-(4-methyl-5-nitrothiazol-2-yl)benzamide (CPD-218)

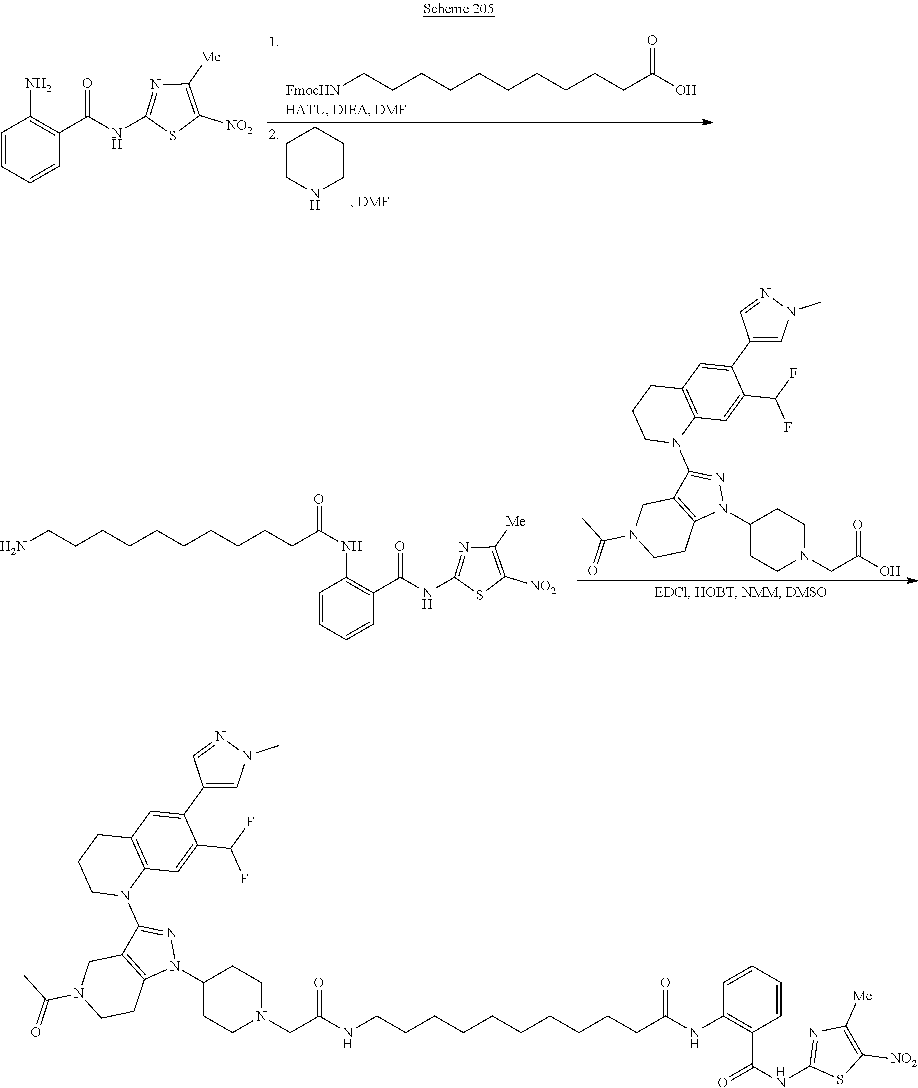

CPD-218 was synthesized following the standard procedure for preparing CPD-209 (14 mg, 70% yield). MS (ESI) m/z=1011.7 [M+H1]⁺.

### Example 206. 5-((3-Aminopropyl)amino)-N-(4,5-dimethylthiazol-2-yl)-2-methylbenzamide (BL1-82)

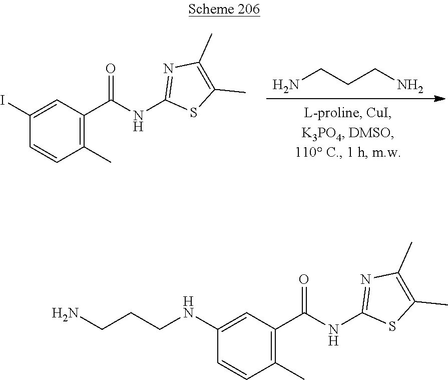 [SMILES: Cc1ccc(I)cc1C(=O)Nc1nc(C)c(C)s1 ; NCCCN ; Cc1ccc(NCCCN)cc1C(=O)Nc1nc(C)c(C)s1]

BL1-82 was synthesized following the standard procedure for preparing BL1-88 (TFA salt, 199 mg, 57% yield) as a yellow solid. ¹HNMR (400 MHz, DMSO-d₆) δ 12.02 (brs, 1H), 7.68 (brs, 3H), 7.00 (d, J=8.4 Hz, 1H), 6.70-6.64 (m, 2H), 3.11 (t, J=6.8 Hz, 2H), 2.90-2.84 (m, 2H), 2.26 (s, 3H), 2.21 (s, 3H), 2.17 (s, 3H), 1.84-1.76 (m 2H). MS (ESI) m/z=319.1 [M+H]⁺.

### Example 207. 5((4-Aminobutyl)amino)-N-(4,5-dimethylthiazol-2-yl)-2-methylbenzamide (BL1-83)

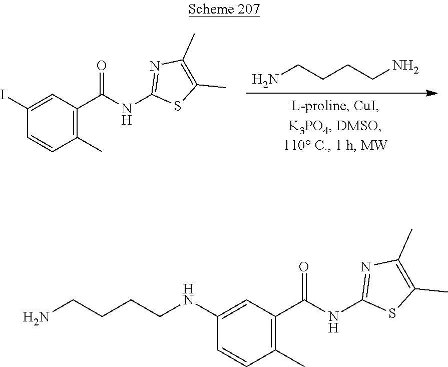 [SMILES: Cc1ccc(I)cc1C(=O)Nc1nc(C)c(C)s1 ; NCCCCN ; Cc1ccc(NCCCCN)cc1C(=O)Nc1nc(C)c(C)s1]

BL1-83 was synthesized following the standard procedure for preparing BL1-88 (TFA salt, 295 mg, 82% yield) as a yellow solid. ¹HNMR (400 MHz, DMSO-d₆) δ 12.07 (brs, 1H), 7.69 (brs, 3H), 7.02 (d, J=8.0 Hz, 1H), 6.74-6.68 (m, 2H), 3.08-3.05 (m, 2H), 2.99-2.95 (m, 2H), 2.26 (s, 3H), 2.22 (s, 3H), 2.17 (s, 3H), 1.63-1.60 (m, 41H). MS (ESI) m/z=333.0 [M+H]⁺.

### Example 208. 5-((2-(2-(2-Aminoethoxy)ethoxy)ethyl)amino)-N-(4,5-dimethylthiazol-2-yl)-2-methylbenzamide (BL1-84)

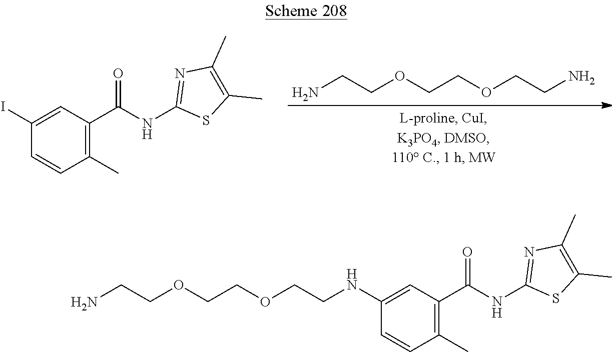 [SMILES: Cc1ccc(I)cc1C(=O)Nc1nc(C)c(C)s1 ; NCCOCCOCCN ; Cc1ccc(NCCOCCOCCN)cc1C(=O)Nc1nc(C)c(C)s1]

BL1-84 was synthesized following the standard procedure for preparing BL1-88 (TFA salt, 350 mg, 65% yield) as a yellow solid. ¹HNMR (400 MHz, DMSO-d₆) δ 12.09 (brs, 1H), 7.77 (brs, 3H), 7.01 (d, J=8.4 Hz, 1H), 6.73 (d, J=2.0 Hz, 1H), 6.70 (dd, J=8.0, 2.4 Hz, 1H), 3.60-3.56 (m, 8H), 3.23 (t, J=6.0 Hz, 2H), 2.98-2.94 (m, 2H), 2.26 (s, 3H), 2.21 (s, 3H), 2.18 (s, 3H). MS (ESI) m/z=393.1 [M+H]⁺.

### Example 209. 3-((8-Aminooctyl)amino)-N-(4,5-dimethylthiazol-2-yl)-2-methylbenzamide (BL1-85)

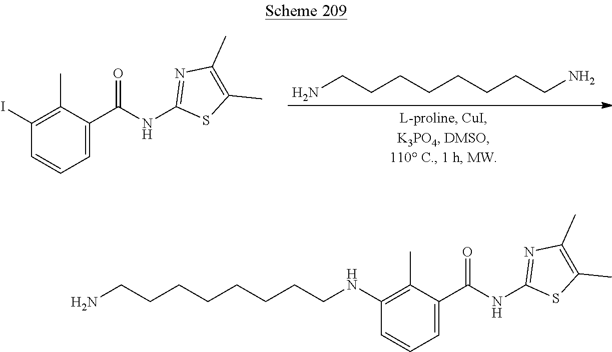 [SMILES: Cc1nc(NC(=O)c2cccc(I)c2C)sc1C ; NCCCCCCCCN ; Cc1nc(NC(=O)c2cccc(NCCCCCCCCN)c2C)sc1C]

BL1-85 was synthesized following the standard procedure for preparing BL1-88 (TFA salt, 95.0 mg, 24% yield) as a yellow solid. ¹HNMR (400 MHz, DMSO-d₆) δ 12.07 (brs, 1H), 7.67 (brs, 3H), 7.10 (t, J=7.8 Hz, 1H), 6.66 (d, J=8.0 Hz, 2H), 3.10 (t, J=6.6 Hz, 2H), 2.79-2.73 (m, 2H), 2.26 (s, 3H), 2.17 (s, 3H), 2.06 (s, 3H), 1.63-1.49 (m, 4H), 1.36-1.34 (m, 8H). MS (ESI) m/z=389.2 [M+H]⁺.

### Example 210. 3-((3-((4,5-Dimethylthiazol-2-yl)carbamoyl)-4-methylphenyl)amino)propanoic acid (BL1-86)

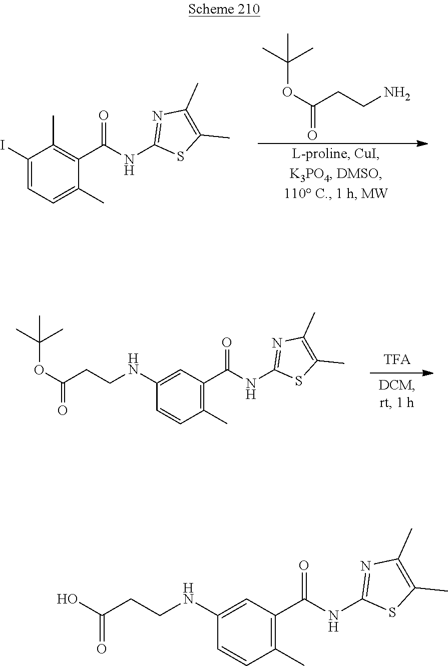

### Step 1. Synthesis of tert-butyl 3-((3-((4,5-dimethylthiazol-2-yl)carbamoyl)-4-methylphenyl)amino)propanoate

A solution of N-(4,5-dimethylthiazol-2-yl)-5-iodo-2-methylbenzamide (400 mg, 1.07 mmol), tert-butyl 3-aminopropanoate (155 mg, 1.07 mmol), L-proline (123 mg, 1.07 mmol), CuI (203 mg, 1.07 mmol) and K₃PO₄ (455 mg, 2.14 mmol) in DMSO (6 mL) was stirred at 110° C. for 1 h in the microwave reactor under inert atmosphere. After cooled to rt, the mixture was purified by reverse-phase HPLC (0.1% TFA) to provide the title compound (300 mg, 71% yield) as a white solid.

### Step 2. Synthesis of 3-((3-((4,5-dimethylthiazol-2-yl)carbamoyl)-4-methylphenyl)amino)propanoic acid

A solution of tert-butyl 3-((3-((4,5-dimethylthiazol-2-yl)carbamoyl)-4-methylphenyl)amino)propanoate (300 mg, 0.771 mmol) in TFA (2 mL) and DCM (2 mL) was stirred at rt for 1 h. Upon completion, the mixture was concentrated. The residue was purified by reverse-phase HPLC (0.1% TFA) to provide the title compound (TFA salt, 260 mg, 76% yield) as a white solid. ¹HNMR (400 MHz, DMSO-d₆) δ 7.05-7.02 (m, 1H), 6.79-6.72 (m, 2H), 3.31-3.28 (m, 2H), 2.54-2.50 (m, 2H), 2.26 (s, 3H), 2.23 (s, 3H), 2.18 (s, 3H). MS (ESI) m/z=334.0 [M+H]⁺.

### Example 211. 3-((2-Aminoethyl)amino)-N-(4,5-dimethylthiazol-2-yl)-2-methylbenzamide (BL1-87)

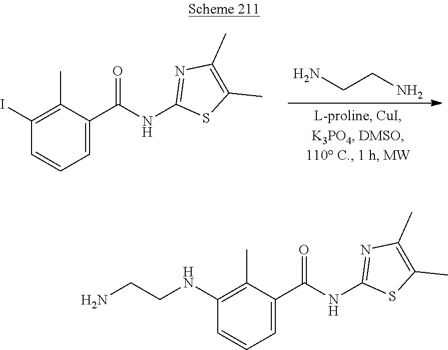

BL1-87 was synthesized following the standard procedure for preparing BL1-88 (TFA salt, 107 mg, 32% yield) as a white solid. ¹HNMR (400 MHz, DMSO-d₆) δ 12.11 (brs, 1H), 7.78 (brs, 3H), 7.13 (t, J=7.6 Hz, 1H), 6.73 (d, J=7.6 Hz, 2H), 3.36 (t, J=6.0 Hz, 2H), 3.04-3.00 (m, 2H), 2.26 (s, 3H), 2.17 (s, 3H), 2.10 (s, 3H). MS (ESI) m/z=305.2 [M+H]⁺.

### Example 212. 5-((2-Aminoethyl)amino)-N-(4,5-dimethylthiazol-2-yl)-2-methylbenzamide (BL1-88)

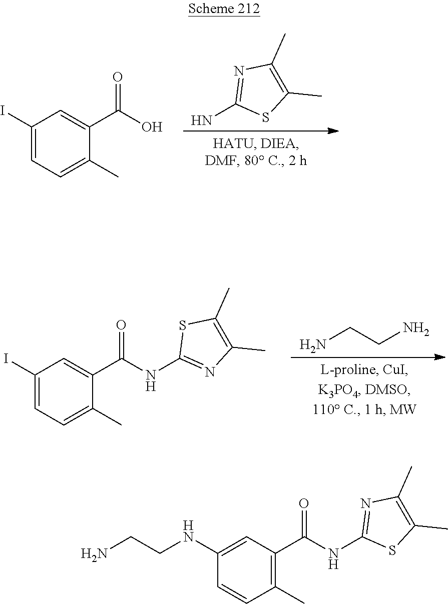

### Step 1. Synthesis of N-(4,5-dimethylthiazol-2-yl)-5-iodo-2-methylbenzamide

A solution of 5-iodo-2-methylbenzoic acid (10.0 g, 38.2 mmol), 4,5-dimethylthiazol-2-amine (7.33 g, 57.3 mmol), HATU (21.8 g, 57.3 mmol) and DIEA (9.86 g, 76.4 mmol) in DMF (100 mL) was stirred at 80° C. for 2 h. After cooled to rt, the mixture was diluted with HCl solution (1 N, 200 mL), and extracted with EtOAc (3×100 mL). The combined organic phase was washed with brine, dried over Na₂SO₄, filtered and concentrated in vacuo. The residue was filtered, and the cake was dried under reduced pressure to provide the title compound (8.56 g, 60% yield) as a white solid. MS (ESI) m/z=372.9 [M+H]⁺.

### Step 2. Synthesis of 5-((2-aminoethyl)amino)-N-(4,5-dimethylthiazol-2-yl)-2-methylbenzamide

A solution of N-(4,5-dimethylthiazol-2-yl)-5-iodo-2-methylbenzamide (300 mg, 0.806 mmol), ethane-1,2-diamine (242 mg, 4.03 mmol), L-proline (93 mg, 0.806 mmol), CuI (153 mg, 0.806 mmol) and K₃PO₄ (342 mg, 1.61 mmol) in DMSO (6 mL) was stirred at 110° C. for 1 h in the microwave reactor under inert atmosphere. After cooled to rt, the mixture was purified by reverse-phase HPLC (0.1% TFA) to provide the title compound (TFA salt, 323 mg, 96% yield) as a yellow solid. ¹HNMR (400 MHz, DMSO-d₆) δ 12.10 (brs, 1H), 7.77 (brs, 3H), 7.03 (d, J=8.4 Hz, 1H), 6.71-6.65 (m, 2H), 3.31-3.27 (m, 2H), 2.99-2.95 (m, 2H), 2.26 (s, 3H), 2.22 (s, 3H), 2.17 (s, 3H). MS (ESI) m/z=305.1 [M+H]⁺.

### Example 213. 5-((6-Aminohexyl)amino)-N-(4,5-dimethylthiazol-2-yl)-2-methylbenzamide (BL1-089)

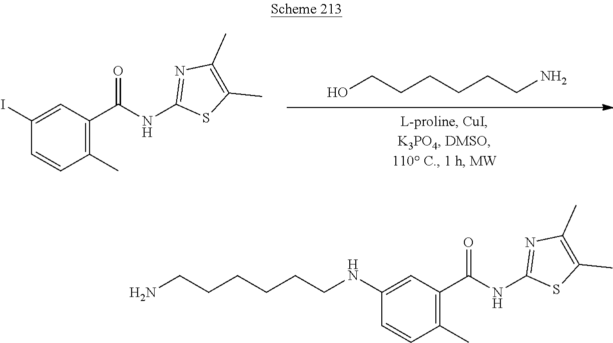 [SMILES: Cc1ccc(I)cc1C(=O)Nc1nc(C)c(C)s1 ; NCCCCCCO ; Cc1ccc(NCCCCCCN)cc1C(=O)Nc1nc(C)c(C)s1]

BL1-89 was synthesized following the standard procedure for preparing BL1-88 (FA salt, 100 mg, 31% yield) as a yellow solid. ¹HNMR (400 MHz, DMSO-d₆) δ 9.12 (brs, 2H), 8.38 (brs, 1H), 6.97 (d, J=8.4 Hz, 1H), 6.65-6.60 (m, 2H), 5.57 (br s, 1H), 3.01-2.00 (m, 2H), 2.74-2.70 (m, 2H), 2.26 (s, 3H), 2.20 (s, 3H), 2.17 (s, 3H), 1.55-1.51 (m, 4H), 1.35-1.34 (m, 4H). MS (ESI) m/z=361.2 [M+H]⁺.

### Example 214. 5-((8-Aminooctyl)amino)-N-(4,5-dimethylthiazol-2-yl)-2-methylbenzamide (BL1-090)

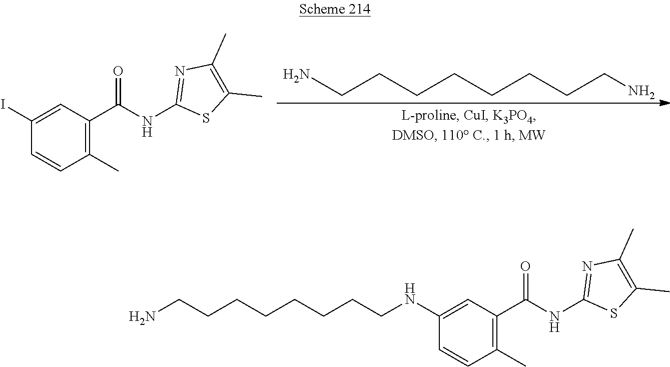 [SMILES: Cc1ccc(I)cc1C(=O)Nc1nc(C)c(C)s1 ; NCCCCCCCCN ; Cc1ccc(NCCCCCCCCN)cc1C(=O)Nc1nc(C)c(C)s1]

BL1-90 was synthesized following the standard procedure for preparing BL1-88 (TFA salt, 400 mg, 78% yield) as a yellow solid. ¹HNMR (400 MHz, DMSO-d₆) δ 12.10 (brs, 1H), 7.68 (brs, 3H), 7.05 (d, J=8.4 Hz, 1H), 6.81-6.75 (m, 2H), 3.05 (d, J=6.0 Hz, 2H), 2.80-2.73 (m, 2H), 2.26 (s, 3H), 2.23 (s, 3H), 2.17 (s, 3H), 1.57-1.48 (m, 4H), 1.35-1.23 (m, 8H). MS (ESI) m/z=389.3 [M+H]⁺.

### Example 215. 5-((2-(2-Aminoethoxy)ethyl)amino)-N-(4,5-dimethylthiazol-2-yl)-2-methylbenzamide (BL1-091)

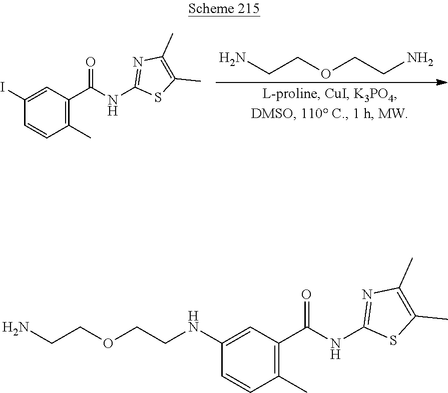 [SMILES: Cc1ccc(I)cc1C(=O)Nc1nc(C)c(C)s1 ; NCCOCCN ; Cc1ccc(NCCOCCN)cc1C(=O)Nc1nc(C)c(C)s1]

BL1-91 was synthesized following the standard procedure for preparing BL1-88 (TFA salt, 460 mg, 93% yield) as a yellow solid. ¹HNMR (400 MHz, DMSO-d₆) δ 12.11 (brs, 1H), 7.85 (brs, 3H), 7.03 (d, J=8.4 Hz, 1H), 6.78-6.72 (m, 2H), 3.62-3.60 (m, 4H), 3.26 (t, J=5.2 Hz, 2H), 3.03-2.99 (m, 2H), 2.26 (s, 3H), 2.23 (s, 3H), 2.18 (s, 3H). MS (ESI) m/z=349.2 [M+H]⁺.

### Example 216. 5-((17-Amino-3,6,9,12,15-pentaoxaheptadecyl)amino)-N-(4,5-dimethylthiazol-2-yl)-2-methylbenzamide (BL1-92)

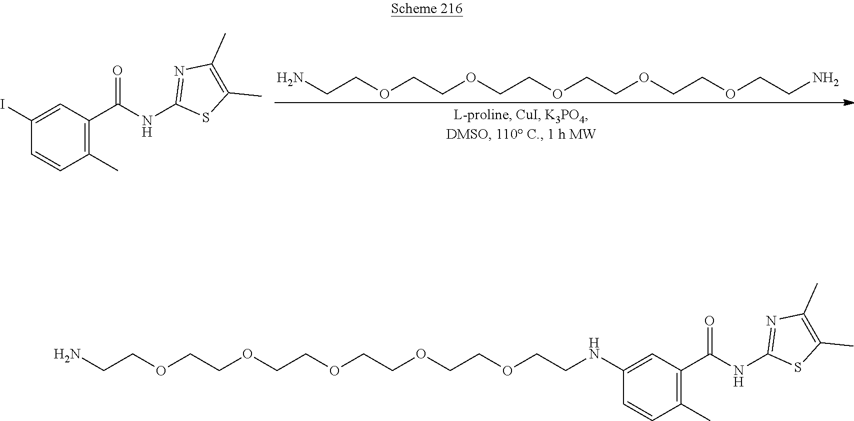

BL1-92 was synthesized following the standard procedure for preparing BL1-88 (TFA salt, 290 mg, 43% yield) as a yellow solid. ¹HNMR (400 MHz, DMSO-d₆) δ 12.08 (brs, 1H), 7.75 (brs, 3H), 7.01 (d, J=8.0 Hz, 1H), 6.77 (d, J=2.4 Hz, 1H), 6.70 (dd, J=8.0, 2.4 Hz, 1H), 3.59-3.50 (m, 20H), 3.23 (t, J=5.6 Hz, 2H), 2.99-2.95 (m, 2H), 2.26 (s, 3H), 2.22 (s, 3H), 2.18 (s, 3H). MS (ESI) m/z=525.2 [M+H]⁺.

### Example 217. 3-((3-Aminopropyl)amino)-N-(4,5-dimethylthiazol-2-yl)-2-methylbenzamide (BL1-93)

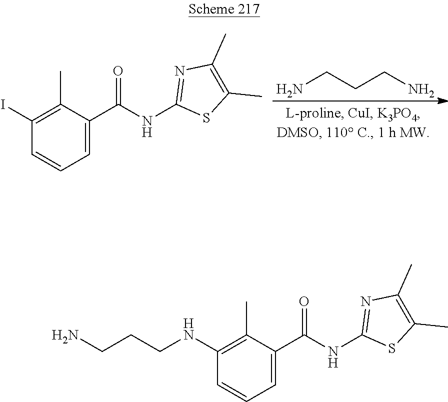

BL1-93 was synthesized following the standard procedure for preparing BL1-88 (TFA salt, 141 mg, 41% yield) as brown oil. ¹HNMR (400 MHz, DMSO-d₆) δ 12.09 (brs, 1H), 7.90 (brs, 1H), 7.77 (brs, 2H), 7.11 (t, J=7.6 Hz, 1H), 6.69 (t, J=9.6 Hz, 2H), 3.21 (t, J=6.8 Hz, 2H), 2.92-2.87 (m, 2H), 2.26 (s, 3H), 2.17 (s, 3H), 2.07 (s, 3H), 1.88-1.81 (m, 2H). MS (ESI) m/z=319.2 [M+H]⁺.

### Example 218. 3-((4-Aminobutyl)amino)-N-(4,5-dimethylthiazol-2-yl)-2-methylbenzamide (BL1-94)

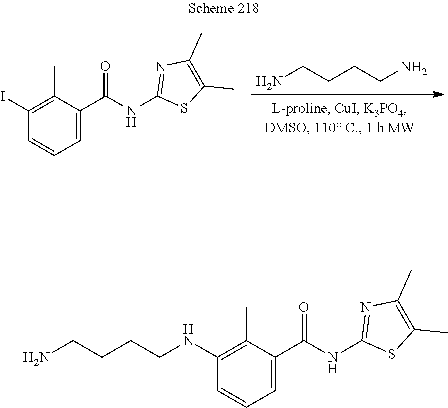

BL1-94 was synthesized following the standard procedure for preparing BL1-88 (TFA salt, 143 mg, 40% yield) as a yellow solid. ¹HNMR (400 MHz, DMSO-d₆) δ 12.11 (brs, 1H), 7.72 (brs, 1H), 7.10 (t, J=7.8 Hz, 1H), 6.69 (t, J=6.8 Hz, 2H), 3.16-3.13 (m, 2H), 2.84-2.82 (m, 2H), 2.27 (s, 3H), 2.17 (s, 3H), 2.07 (s, 3H), 1.65-1.61 (m, 4H). MS (ESI) m/z=333.1 [M+H]⁺.

### Example 219. 3-((5-Aminopentyl)amino)-N-(4,5-dimethylthiazol-2-yl)-2-methylbenzamide (BL1-95)

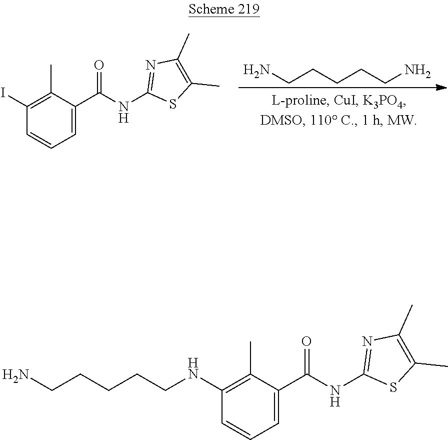 [SMILES: Cc1nc(NC(=O)c2cccc(I)c2C)sc1C ; NCCCCCN ; Cc1nc(NC(=O)c2cccc(NCCCCCN)c2C)sc1C]

BL1-95 was synthesized following the standard procedure for preparing BL1-88 (TFA salt, 123 mg, 33% yield) as a yellow solid. ¹HNMR (400 MHz, DMSO-d₆) δ 12.07 (brs, 1H), 7.69 (brs, 3H), 7.10 (t, J=7.8 Hz, 1H), 6.66 (d, J=8.0 Hz, 2H), 3.11 (t, J=6.8 Hz, 2H), 2.82-2.75 (m, 2H), 2.26 (s, 3H), 2.19 (s, 3H), 2.07 (s, 3H), 1.64-1.54 (m, 4H), 1.44-1.37 (m, 2H). MS (ESI) m/z=347.2 [M+H]⁺.

### Example 220. 3-((6-Aminohexyl)amino)-N-(4,5-dimethylthiazol-2-yl)-2-methylbenzamide (BL1-96)

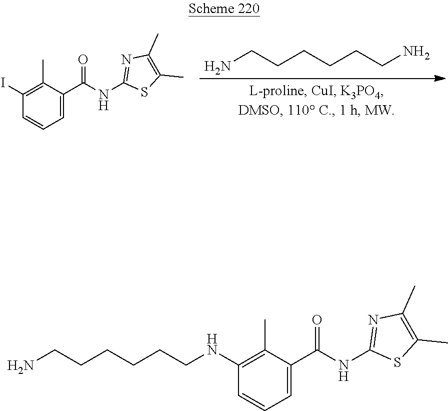 [SMILES: Cc1nc(NC(=O)c2cccc(I)c2C)sc1C ; NCCCCCCN ; Cc1nc(NC(=O)c2cccc(NCCCCCCN)c2C)sc1C]

BL1-96 was synthesized following the standard procedure for preparing BL1-88 (TFA salt, 90 mg, 24% yield) as a yellow solid. ¹HNMR (400 MHz, DMSO-d₆) δ 12.07 (brs, 1H), 7.72 (brs, 3H), 7.10 (t, J=7.8 Hz, 1H), 6.68-6.65 (m, 2H), 3.11 (t, J=7.2 Hz, 2H), 2.80-2.75 (m, 2H), 2.26 (s, 3H), 2.17 (s, 3H), 2.06 (s, 3H), 1.61-1.54 (m, 4H), 1.37-1.35 (m, 4H). MS (ESI) m/z=361.1 [M+H]⁺.

### Example 221. 3-((7-Aminoheptyl)amino)-N-(4,5-dimethylthiazol-2-yl)-2-methylbenzamide (BL1-97)

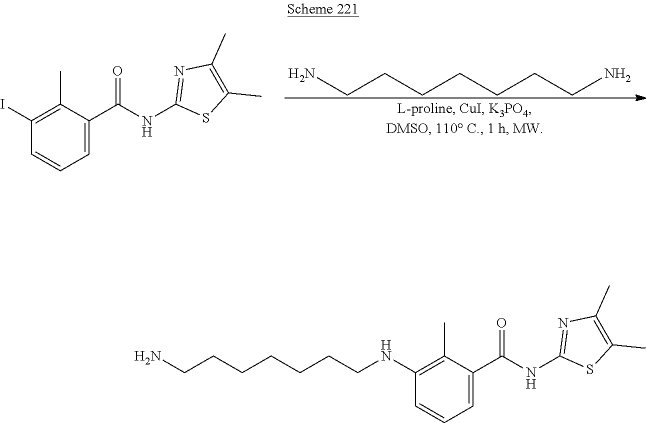 [SMILES: Cc1nc(NC(=O)c2cccc(I)c2C)sc1C ; NCCCCCCCN ; Cc1nc(NC(=O)c2cccc(NCCCCCCCN)c2C)sc1C]

BL1-97 was synthesized following the standard procedure for preparing BL1-88 (TFA salt, 150 mg, 38% yield) as a yellow solid. ¹HNMR (400 MHz, DMSO-d₆) δ 12.07 (brs, 1H), 7.69 (brs, 3H), 7.10 (t, J=7.8 Hz, 1H), 6.71-6.67 (m, 2H), 3.11 (t, J=7.2 Hz, 2H), 2.81-2.75 (m, 2H), 2.26 (s, 3H), 2.17 (s, 3H), 2.06 (s, 3H), 1.61-1.51 (m, 4H), 1.41-1.27 (m, 6H). MS (ESI) m/z=375.2 [M+H]⁺.

### Example 222. 3-((2-(2-Aminoethoxy)ethyl)amino)-N-(4,5-dimethylthiazol-2-yl)-2-methylbenzamide (BL1-98)

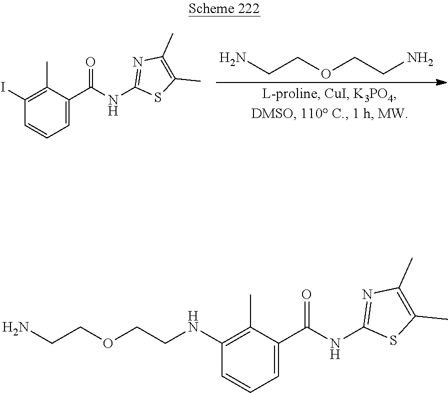 [SMILES: Cc1nc(NC(=O)c2cccc(I)c2C)sc1C ; NCCOCCN ; Cc1nc(NC(=O)c2cccc(NCCOCCN)c2C)sc1C]

BL1-98 was synthesized following the standard procedure for preparing BL1-88 (TFA salt, 100 mg, yield: 27%) as a yellow solid. ¹HNMR (400 MHz, DMSO-d₆) δ 12.09 (brs, 1H), 7.79 (brs, 3H), 7.11 (t, J=7.8 Hz, 1H), 6.74-6.69 (m, 2H), 3.62-3.60 (m, 4H), 3.33 (t, J=5.8 Hz, 2H), 3.03-2.99 (m, 2H), 2.26 (s, 3H), 2.17 (s, 3H), 2.07 (s, 3H). MS (ESI) m/z=349.2 [M+H]⁺.

### Example 223. 3-((2-(2-(2-Aminoethoxy)ethoxy)ethyl)amino)-N-(4,5-dimethylthiazol-2-yl)-2-methylbenzamide (BL1-99)

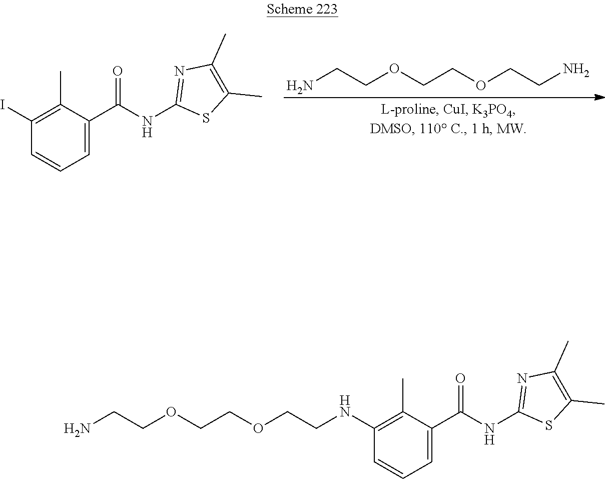 [SMILES: Cc1nc(NC(=O)c2cccc(I)c2C)sc1C ; NCCOCCOCCN ; Cc1nc(NC(=O)c2cccc(NCCOCCOCCN)c2C)sc1C]

BL1-99 was synthesized following the standard procedure for preparing BL1-88 (TFA salt, 110 mg, 27% yield) as a yellow solid. ¹HNMR (400 MHz, DMSO-d₆) δ 12.08 (brs, 1H), 7.79 (brs, 3H), 7.11 (t, J=7.8 Hz, 1H), 6.73-6.78 (m, 2H), 3.64-3.58 (m, 8H), 3.31-3.28 (m, 2H), 2.98-2.95 (m, 2H), 2.26 (s, 3H), 2.17 (s, 3H), 2.06 (s, 3H). MS (ESI) m/z=393.2 [M+H]⁺.

### Example 224. 3-((2-(2-(2-(2-Aminoethoxy)ethoxy)ethoxy)ethyl)amino)-N-(4,5-dimethylthiazol-2-yl)-2-methylbenzamide (BL1-100)

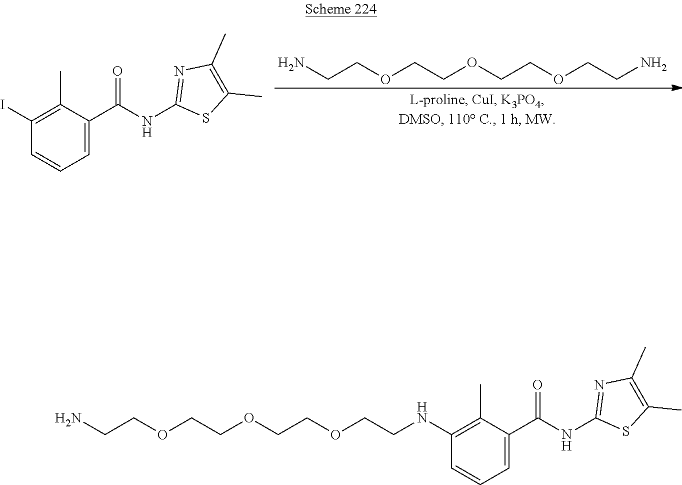 [SMILES: Cc1nc(NC(=O)c2cccc(I)c2C)sc1C ; NCCOCCOCCOCCN ; Cc1nc(NC(=O)c2cccc(NCCOCCOCCOCCN)c2C)sc1C]

BL1-100 was synthesized following the standard procedure for preparing BL1-88 (TFA salt, 110 mg, 23% yield) as a yellow solid. ¹HNMR (400 MHz, DMSO-d₆) δ 12.06 (brs, 1H), 7.75 (brs, 3H), 7.11 (t, J=8.0 Hz, 1H), 6.72-6.67 (m, 2H), 3.63-3.56 (m, 12H), 3.29 (t, J=6.0 Hz, 2H), 2.99-2.95 (m, 2H), 2.26 (s, 3H), 2.17 (s, 3H), 2.06 (s, 3H). MS (ESI) m/z=437.1 [M+H]⁺.

### Example 225. 3-((14-Amino-3,6,9,12-tetraoxatetradecyl)amino)-N-(4,5-dimethylthiazol-2-yl)-2-methylbenzamide (BL1-101)

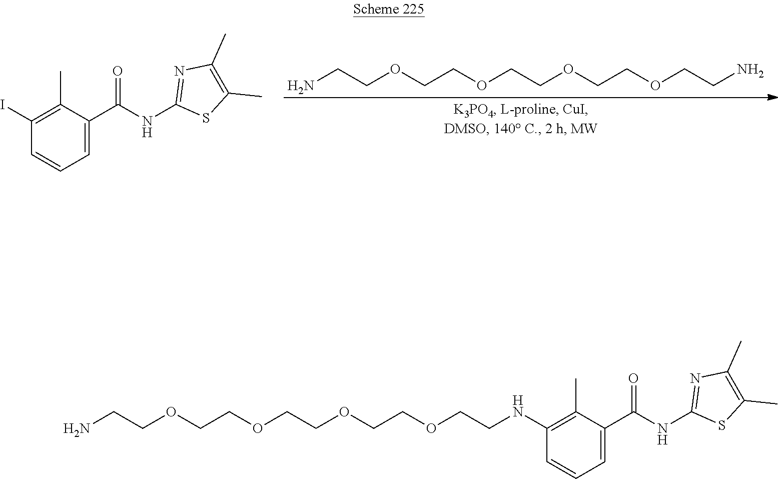 [SMILES: Cc1nc(NC(=O)c2cccc(I)c2C)sc1C ; NCCOCCOCCOCCOCCN ; Cc1nc(NC(=O)c2cccc(NCCOCCOCCOCCOCCN)c2C)sc1C]

BL1-101 was synthesized following the standard procedure for preparing BL1-88 (TFA salt, 90.0 mg, 18% yield) as brown oil. ¹HNMR (400 MHz, DMSO-d₆) δ 12.08 (brs, 1H), 7.73 (brs, 3H), 7.10 (t, J=8.0 Hz, 1H), 6.69 (dd, J=14.4 Hz, 8.4 Hz, 2H), 3.63-3.53 (m, 16H), 3.29 (t, J=6.0 Hz, 2H), 2.99-2.94 (m, 2H), 2.26 (s, 3H), 2.17 (s, 3H), 2.06 (s, 3H). MS (ESI) m/z=481.2 [M+H]⁺.

### Example 226. 3-((17-Amino-3,6,9,12,15-pentaoxaheptadecyl)amino)-N-(4,5-dimethylthiazol-2-yl)-2-methylbenzamide (BL1-102)

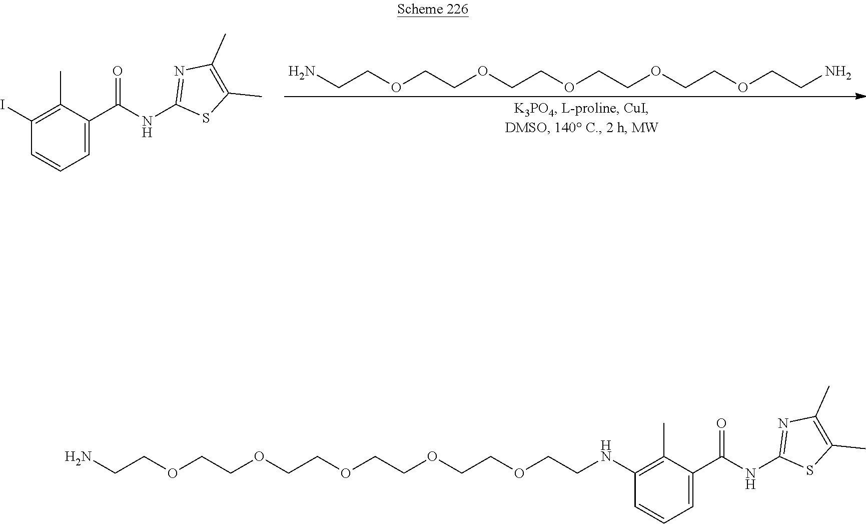 [SMILES: Cc1nc(NC(=O)c2cccc(I)c2C)sc1C ; NCCOCCOCCOCCOCCOCCN ; Cc1nc(NC(=O)c2cccc(NCCOCCOCCOCCOCCOCCN)c2C)sc1C]

BL1-102 was synthesized following the standard procedure for preparing BL1-88 (TFA salt, 140 mg, 27% yield) as brown oil. ¹HNMR (400 MHz, DMSO-d₆) δ 12.07 (brs, 1H), 7.72 (brs, 3H), 7.10 (t, J=7.8 Hz, 1H), 6.69 (dd, J=15.2 Hz, 8.0 Hz, 2H), 3.63-3.53 (m, 20H), 3.29 (t, J=6.0 Hz, 2H), 2.99-2.95 (m, 2H), 2.26 (s, 3H), 2.17 (s, 3H), 2.05 (s, 3H). MS (ESI) m/z=525.3 [M+H]⁺.

### Example 227. 5-((3-((4,5-Dimethylthiazol-2-yl)carbamoyl)-4-methylphenyl)amino)pentanoic acid (BL1-103)

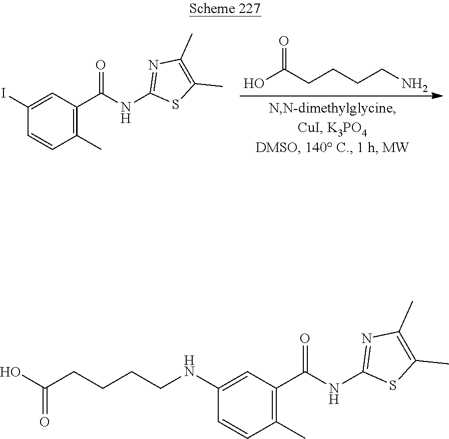

A solution of N-(4,5-dimethylthiazol-2-yl)-5-iodo-2-methylbenzamide (400 mg, 1.07 mmol), 5-aminopentanoic acid (626 mg, 5.35 mmol), N,N-Dimethylglycine (110 mg, 1.07 mmol), CuI (203 mg, 1.07 mmol) and K₃PO₄ (455 mg, 2.14 mmol) in DMSO (6 mL) was stirred at 140° C. for 1 h in the microwave reactor under inert atmosphere. After cooled to rt, the mixture was purified by reverse-phase HPLC (0.1% TFA) to provide the title compound (TFA salt, 100 mg, 20% yield) as a yellow solid. ¹H NMR (400 MHz, DMSO-d₆) δ 7.01 (d, J=8.0 Hz, 1H), 6.75-6.68 (m, 2H), 3.06-3.04 (m, 2H), 2.27-2.19 (m, 11H), 1.58-1.57 (m, 4H). MS (ESI) m/z=362.1 [M+H]⁺.

### Example 228. 7-((3-((4,5-Dimethylthiazol-2-yl)carbamoyl)-4-methylphenyl)amino)heptanoic acid (BL1-104)

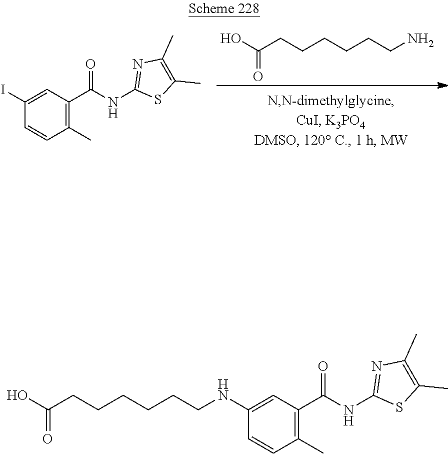 [SMILES: Cc1ccc(I)cc1C(=O)Nc1nc(C)c(C)s1 ; NCCCCCCC(=O)O ; Cc1ccc(NCCCCCCC(=O)O)cc1C(=O)Nc1nc(C)c(C)s1]

BL1-104 was synthesized following the standard procedure for preparing BL1-103 (TFA salt, 148 mg, 28% yield) as a yellow solid. ¹HNMR (400 MHz, DMSO-d₆) δ 12.03 (brs, 1H), 7.10 (d, J=7.6 Hz, 1H), 6.91-6.84 (m, 2H), 3.10-3.07 (m, 2H), 2.26-2.18 (m, 11H), 1.57-1.47 (m, 4H), 1.39-1.27 (m, 4H). MS (ESI) m/z=390.1 [M+H]⁺.

### Example 229. 3-(2-((3-((4,5-Dimethylthiazol-2-yl)carbamoyl)-4-methylphenyl)amino)ethoxy)propanoic acid (BL1-105)

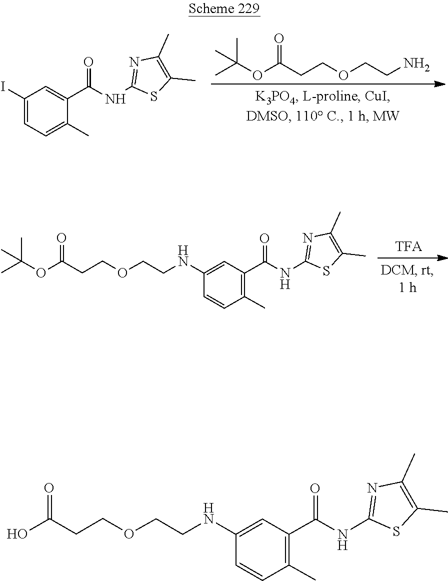

### Step 1. Synthesis of tert-butyl 3-(2-((3-((4,5-dimethylthiazol-2-yl)carbamoyl)-4-methylphenyl)amino)ethoxy)propanoate

A solution of N-(4,5-dimethylthiazol-2-yl)-5-iodo-2-methylbenzamide (400 mg, 1.07 mmol), tert-butyl 3-(2-aminoethoxy)propanoate (405 mg, 2.14 mmol), L-proline (123 mg, 1.07 mmol), CuI (203 mg, 1.07 mmol) and K₃PO₄ (455 mg, 2.14 mmol) in DMSO (6 mL) was stirred at 110° C. for 1 h in the microwave reactor under N₂. After cooled to rt, the mixture was purified by reverse-phase HPLC (0.1% TFA) to provide the title compound (400 mg, 86% yield) as a yellow solid.

### Step 2. Synthesis of 3-(2-((3-((4,5-dimethylthiazol-2-yl)carbamoyl)-4-methylphenyl)amino)ethoxy)propanoic acid

A solution of tert-butyl 3-(2-((3-((4,5-dimethylthiazol-2-yl)carbamoyl)-4-methylphenyl)amino)ethoxy)propanoate (400 mg, 0.923 mmol) in TFA (2 mL) and DCM (2 mL) was stirred at rt for 1 h. Upon completion, the mixture was concentrated. The residue was purified by reverse-phase HPLC (0.1% TFA) to provide the title compound (TFA salt, 300 mg, 66% yield) as a yellow solid. ¹HNMR (400 MHz, DMSO-d₆) δ 7.09 (d, J=8.0 Hz, 1H), 6.92 (s, 1H), 6.86-6.84 (m, 1H), 3.64 (d, J=6.4 Hz, 2H), 3.56 (d, J=6.4 Hz, 2H), 3.37 (d, J=5.6 Hz, 2H), 2.49-2.47 (m, 2H), 2.27 (s, 3H), 2.25 (s, 3H), 2.19 (s, 3H). MS (ESI) m/z=378.1 [M+H]⁺.

### Example 230. 3-(2-(2-(2-((3-((4,5-Dimethylthiazol-2-yl)carbamoyl)-4-methylphenyl)amino)ethoxy)ethoxy)ethoxy)propanoic acid (BL1-106)

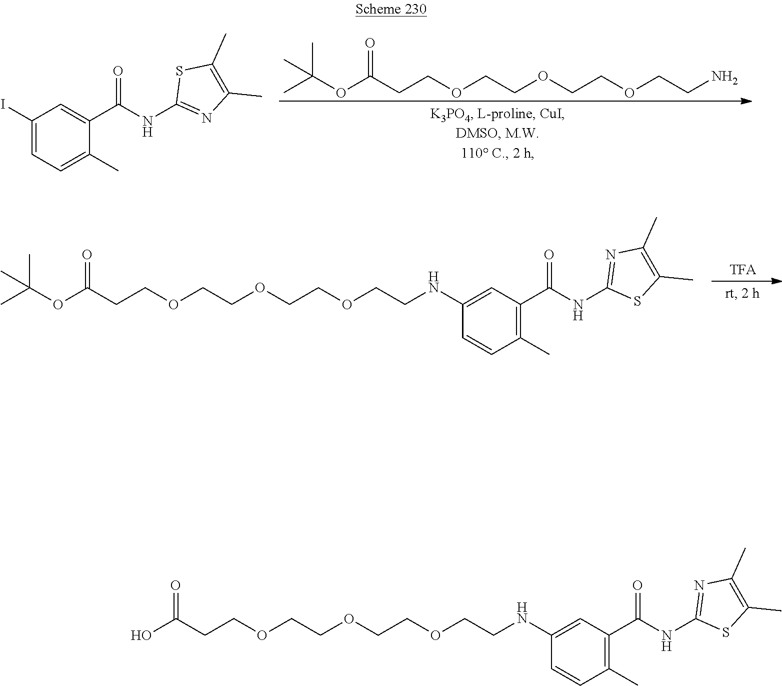

BL1-106 was synthesized following the standard procedure for preparing BL1-105 (TFA salt, 110 mg, 21% yield over 2 steps) as a yellow solid. ¹HNMR (400 MHz, DMSO-d₆) δ 7.04 (d, J=8.4 Hz, 1H), 6.83 (s, 1H), 6.78-6.76 (m, 1H), 3.80-3.47 (m, 12H), 3.25 (t, J=5.8 Hz, 2H), 2.43 (t, J=6.4 Hz, 2H), 2.27 (s, 3H), 2.23 (s, 3H), 2.18 (s, 3H). MS (ESI) m/z=466.2 [M+H]⁺.

### Example 231. 3-((3-((4,5-Dimethylthiazol-2-yl)carbamoyl)-2-methylphenyl)amino)propanoic acid (BL1-107)

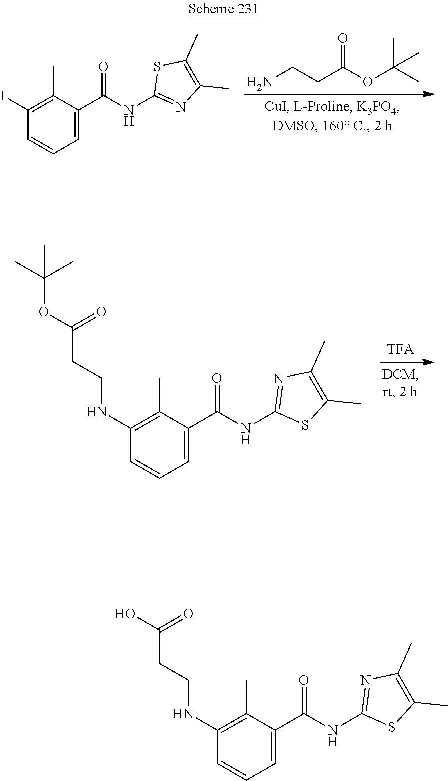

BL1-107 was synthesized following the standard procedure for preparing BL1-105 (110 mg, 13% yield over 2 steps) as a yellow solid. ¹HNMR (400 MHz, DMSO-d₆) δ 7.41 (s, 1H), 7.28 (s, 1H), 7.15-7.10 (m, 2H), 6.73-6.68 (m, 2H), 3.37-3.33 (m, 2H), 2.58-2.55 (m, 2H), 2.26 (s, 3H), 2.16 (s, 3H), 2.05 (s, 3H). MS (ESI) m/z=334.0 [M+H]⁺.

### Example 232. 2-(9-Acetamidononanamido)-N-(4,5-dimethylthiazol-2-yl)benzamide (BL1-108)

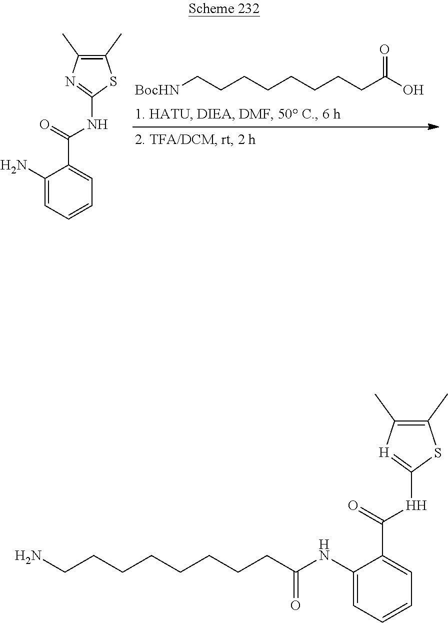

BL1-108 was synthesized following the standard procedure for preparing BL1-144 (736 mg, 84% yield over 2 steps) as a white solid. MS (ESI) m/z=403.2 [M+H]⁺.

### Example 233. 2-(3-(2-(2-(2-Aminoethoxy)ethoxy)ethoxy)propanamido)-N-(4,5-dimethylthiazol-2-yl)benzamide (BL1-109)

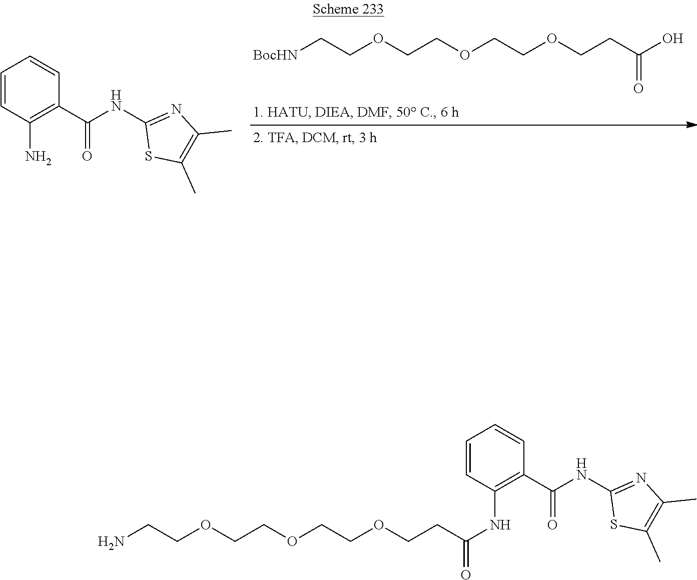 [SMILES: Cc1nc(NC(=O)c2ccccc2N)sc1C ; CC(C)(C)OC(=O)NCCOCCOCCOCCC(=O)O ; Cc1nc(NC(=O)c2ccccc2NC(=O)CCOCCOCCOCCN)sc1C]

BL1-109 was synthesized following the standard procedure for preparing BL1-144 (500 mg, 54% yield over 2 steps) as a yellow solid. MS (ESI) m/z=450.9 [M+H]⁺.

### Example 234. 2-(3-(2-(2-Aminoethoxy)ethoxy)propanamido)-N-(4,5-dimethylthiazol-2-yl)benzamide (BL1-110)

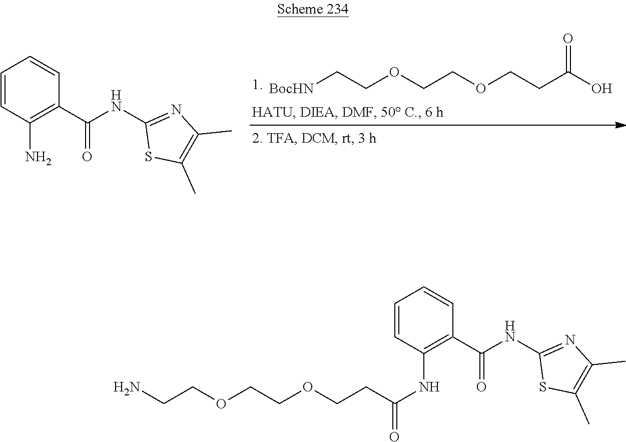 [SMILES: Cc1nc(NC(=O)c2ccccc2N)sc1C ; CC(C)(C)OC(=O)NCCOCCOCCC(=O)O ; Cc1nc(NC(=O)c2ccccc2NC(=O)CCOCCOCCN)sc1C]

BL1-110 was synthesized following the standard procedure for preparing BL1-144 (390 mg, 76% yield over 2 steps) as a yellow solid. MS (ESI) m/z=406.9 [M+H]⁺.

### Example 235. 5-((5-Aminopentyl)amino)-N-(4,5-dimethylthiazol-2-yl)-2-methylbenzamide (BL1-111)

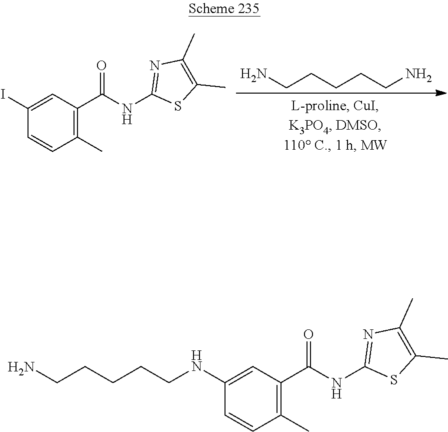

BL1-111 was synthesized following the standard procedure for preparing BL1-88 (TFA salt, 400 mg, 81% yield) as a yellow solid. ¹HNMR (400 MHz, DMSO-d₆) δ 12.03 (brs, 1H), 7.66 (brs, 3H), 7.02 (d, J=8.0 Hz, 1H), 6.73-6.68 (m, 2H), 3.05-3.02 (m, 2H), 2.81-2.76 (m, 2H), 2.26 (s, 3H), 2.22 (s, 3H), 2.18 (s, 3H), 1.63-1.52 (m, 4H), 1.43-1.37 (m, 2H). MS (ESI) m/z=347.2 [M+H]⁺.

### Example 236. 5-((7-Aminoheptyl)amino)-N-(4,5-dimethylthiazol-2-yl)-2-methylbenzamide (BL1-112)

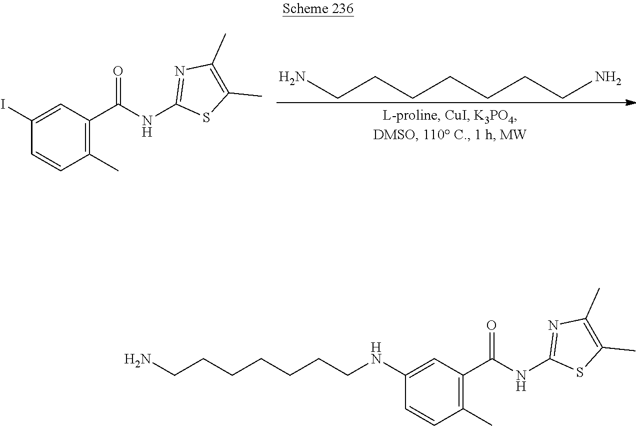

BL1-112 was synthesized following the standard procedure for preparing BL1-88 (TFA salt, 350 mg, 67% yield) as a yellow solid. ¹HNMR (400 MHz, DMSO-d₆) δ 12.05 (brs, 1H), 7.65 (brs, 3H), 7.02 (d, J=8.0 Hz, 1H), 6.75-6.71 (m, 2H), 3.05-3.02 (m, 2H), 2.81-2.73 (m, 2H), 2.26 (s, 3H), 2.22 (s, 3H), 2.17 (s, 3H), 1.56-1.52 (m, 4H), 1.35-1.31 (m, 2H). MS (ESI) m/z=375.2 [M+H]⁺.

### Example 237. 5-((14-Amino-3,6,9,12-tetraoxatetradecyl)amino)-N-(4,5-dimethylthiazol-2-yl)-2-methylbenzamide (BL1-113)

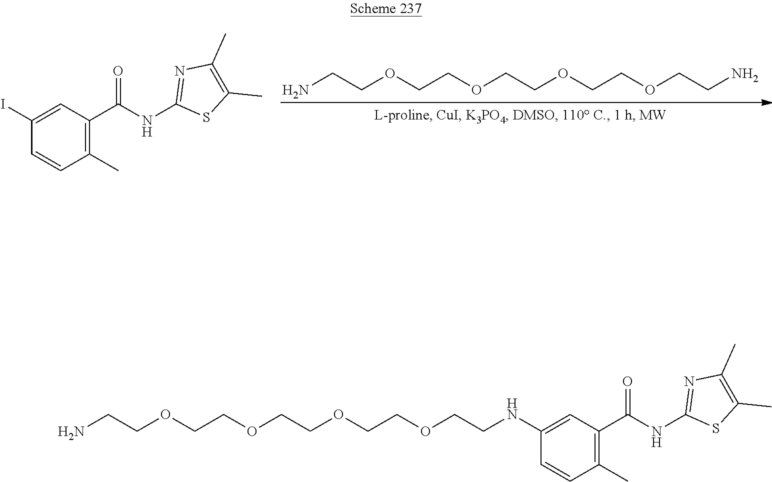

BL1-113 was synthesized following the standard procedure for preparing BL1-88 (TFA salt, 400 mg, 63% yield) as a yellow solid. ¹HNMR (400 MHz, DMSO-d₆) δ 7.77 (brs, 3H), 7.06 (d, J=8.4 Hz, 1H), 6.86 (d, J=2.0 Hz, 1H), 6.70 (dd, J=8.0, 2.0 Hz, 1H), 3.59-3.52 (m, 16H), 3.27 (t, J=5.6 Hz, 2H), 2.99-2.95 (m, 2H), 2.26 (s, 3H), 2.24 (s, 3H), 2.18 (s, 3H). MS (ESI) m/z=481.2 [M+H]⁺.

### Example 238. 1-((3-((4,5-Dimethylthiazol-2-yl)carbamoyl)-4-methylphenyl)amino)-3,6,9,12-tetraoxapentadecan-15-oic acid (BL1-114)

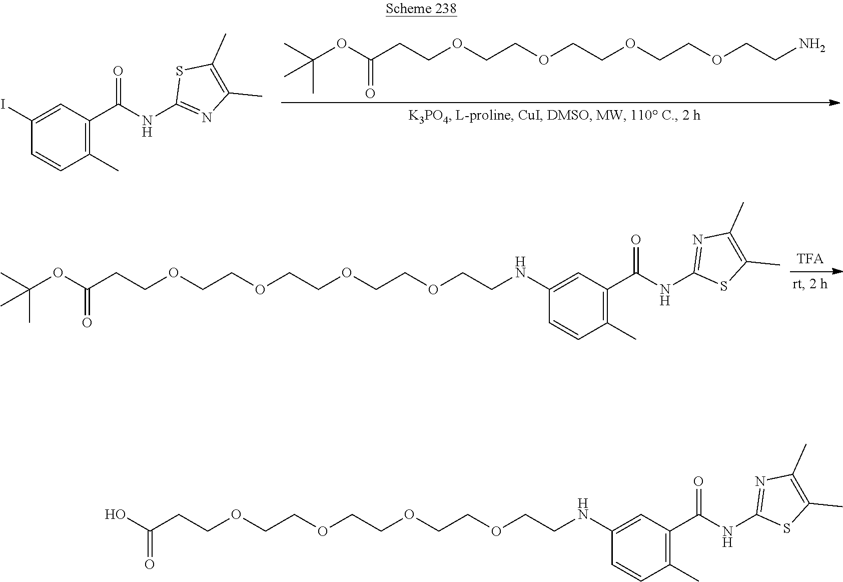

BL1-114 was synthesized following the standard procedure for preparing BL1-105 (TFA salt, 240 mg, 48% yield over 2 steps) as a yellow solid. ¹HNMR (400 MHz, DMSO-d₆) δ 7.10 (d, J=8.4 Hz, 1H), 6.94 (s, 1H), 6.87 (d, J=8.0 Hz, 1H), 3.60-3.47 (m, 16H), 3.30 (t, J=5.6 Hz, 2H), 2.43 (t, J=6.4 Hz, 2H), 2.27 (s, 3H), 2.26 (s, 3H), 2.18 (s, 3H). MS (ESI) m/z=510.2 [M+H]⁺.

### Example 239. 3-(2-((3-((4,5-Dimethylthiazol-2-yl)carbamoyl)-2-methylphenyl)amino)ethoxy)propanoic acid (BL1-115)

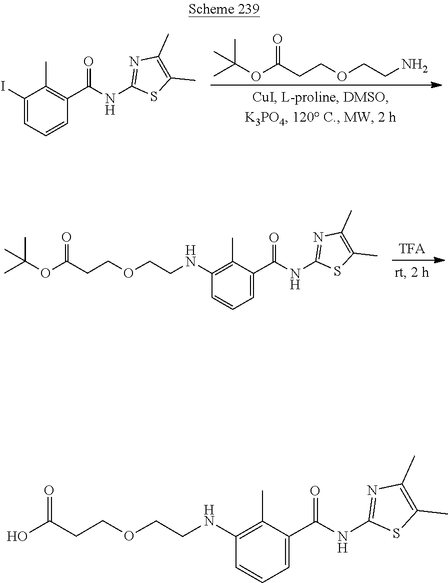

BL1-115 was synthesized following the standard procedure for preparing BL1-105 (TFA salt, 40 mg, 5% yield over 2 steps) as a yellow solid. ¹HNMR (400 MHz, DMSO-d₆) δ 12.09 (brs, 1H), 7.11 (t, J=7.6 Hz, 1H), 6.72-6.67 (m, 2H), 3.65 (t, J=6.2 Hz, 2H), 3.60 (t, J=6.2 Hz, 2H), 3.28 (t, J=6.2 Hz, 2H), 2.48 (t, J=6.2 Hz, 2H), 2.27 (s, 3H), 2.17 (s, 3H), 2.06 (s, 3H). MS (ESI) m/z=378.1 [M+H]⁺.

### Example 240. 3-(2-(2-((3-((4,5-Dimethylthiazol-2-yl)carbamoyl)-2-methylphenyl)amino)ethoxy)ethoxy)propanoic acid (BL1-116)

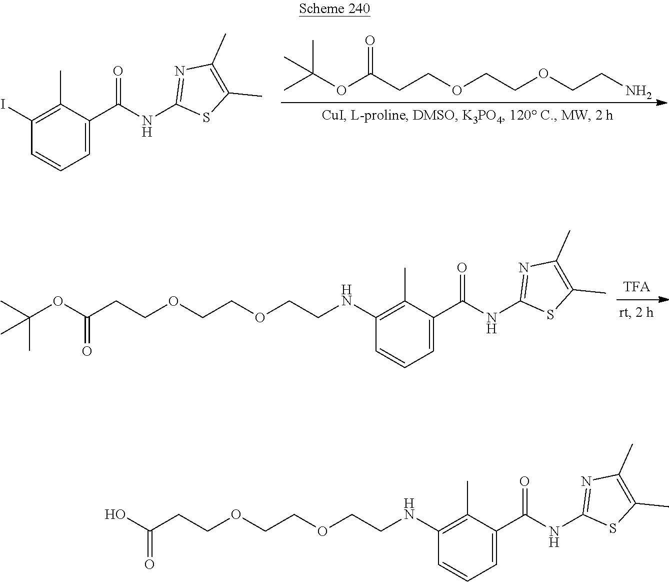

BL1-116 was synthesized following the standard procedure for preparing BL1-105 (TFA salt, 50 mg, 6% yield over 2 steps) as a yellow solid. ¹HNMR (400 MHz, DMSO-d₆) δ 12.10 (brs, 1H), 7.11 (t, J=7.6 Hz, 11H), 6.74-6.67 (m, 2H), 3.64-3.60 (m, 4H), 3.57-3.51 (m, 4H), 3.29 (t, J=5.8 Hz, 2H), 2.45 (t, J=6.2 Hz, 2H), 2.27 (s, 3H), 2.17 (s, 3H), 2.06 (s, 3H). MS (ESI) m/z=422.1 [M+H]⁺.

### Example 241. 3-(2-(2-(2-((3-((4,5-Dimethylthiazol-2-yl)carbamoyl)-2-methylphenyl)amino)ethoxy)ethoxy)ethoxy)propanoic acid (BL1-117)

### Scheme 241

BL1-117 was synthesized following the standard procedure for preparing BL1-105 (TFA salt, 30 mg, 3% yield over 2 steps) as a yellow solid. ¹HNMR (400 MHz, DMSO-d₆) δ 12.07 (brs, 1H), 7.10 (t, J=7.8 Hz, 1H), 6.73-6.67 (m, 2H), 3.62-3.48 (m, 12H), 3.29 (t, J=6.0 Hz, 2H), 2.43 (t, J=6.4 Hz, 2H), 2.26 (s, 3H), 2.17 (s, 3H), 2.05 (s, 3H). MS (ESI) m/z=466.1 [M+H]⁺.

### Example 242. 1-((3-((4,5-Dimethylthiazol-2-yl)carbamoyl)-2-methylphenyl)amino)-3,6,9,12-tetraoxapentadecan-15-oic acid (BL1-118)

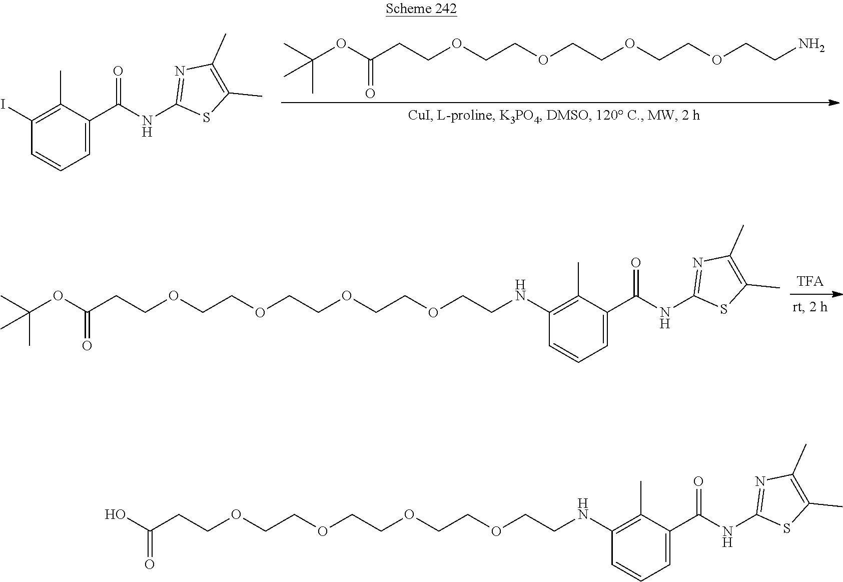

BL1-118 was synthesized following the standard procedure for preparing BL1-105 (TFA salt, 30 mg, 3% yield over 2 steps) as a yellow solid. ¹HNMR (400 MHz, DMSO-d₆) δ 12.09 (brs, 1H), 7.10 (t, J=8.0 Hz, 1H), 6.73-6.67 (m, 2H), 3.62-3.48 (m, 16H), 3.29 (t, J=6.2 Hz, 2H), 2.43 (t, J=6.4 Hz, 2H), 2.26 (s, 3H), 2.16 (s, 3H), 2.05 (s, 3H). MS (ESI) m/z=510.2 [M+H]⁺.

### Example 243. (3-((4,5-Dimethylthiazol-2-yl)carbamoyl)-2-methylphenyl)glycine (BL1-119)

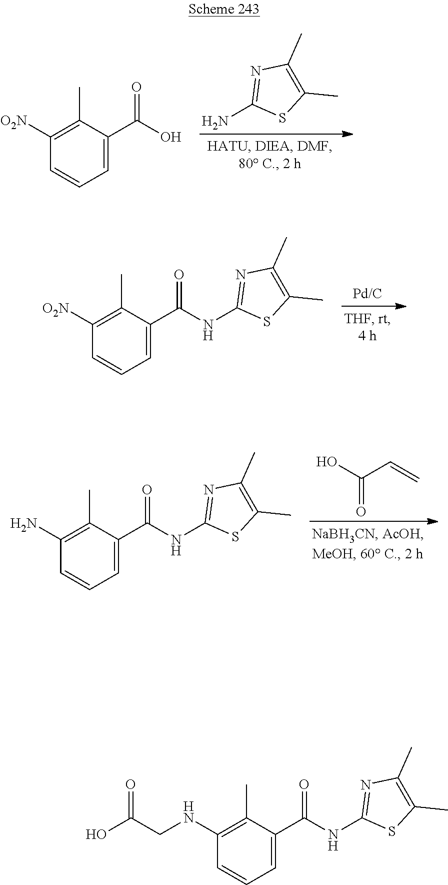

### Step 1. Synthesis of N-(4,5-dimethylthiazol-2-yl)-2-methyl-3-nitrobenzamide

A solution of 2-methyl-3-nitrobenzoic acid (1.00 g, 5.52 mmol), 4,5-dimethylthiazol-2-amine (1.41 g, 11.0 mmol), HATU (3.15 g, 8.28 mmol) and DIEA (1.42 g, 11.0 mmol) in DMF (10 mL) was stirred at 80° C. for 2 h. After cooled to rt, the mixture was diluted with water (50 mL) and extracted with EtOAc (50 mL×2). The combined organic phase was washed with brine (50 mL×2), dried over Na₂SO₄, filtered and concentrated in vacuo. The residue was purified by silica gel chromatography (petroleum ether/EtOAc=5:1) to provide the title compound (800 mg, 50% yield) as a yellow solid. MS (ESI) m/z=292.0 [M+H]⁺.

### Step 2. Synthesis of 3-amino-N-(4,5-dimethylthiazol-2-yl)-2-methylbenzamide

A solution of N-(4,5-dimethylthiazol-2-yl)-2-methyl-3-nitrobenzamide (800 mg, 2.75 mmol) and Pd/C (10%, 100 mg) in THF (8 mL) was stirred at rt for 4 h under H₂ atmosphere. Upon completion, the mixture was filtered, and the filtrate was concentrated in vacuo. The residue was purified by silica gel chromatography (petroleum ether/EtOAc=3:1) to provide the title compound (500 mg, 70% yield) as a white solid. MS (ESI) m/z=262.0 [M+H]⁺.

### Step 3. Synthesis of (3-((4,5-dimethylthiazol-2-yl)carbamoyl)-2-methylphenyl)glycine

A solution of 3-amino-N-(4,5-dimethylthiazol-2-yl)-2-methylbenzamide (300 mg, 1.15 mmol), glyoxylic acid (50 wt. % in water, 851 mg, 5.75 mmol), NaBH₃CN (362 mg, 5.75 mmol) and AcOH (69 mg, 1.15 mmol) in MeOH (3 mL) was stirred at 60° C. for 2 h. After cooled to rt, the mixture was filtered, and the cake was washed with MeOH to provide the title compound (120 mg, 33% yield) as a white solid. ¹HNMR (400 MHz, DMSO-d₆) δ 12.11 (brs, 1H), 7.09 (t, J=8.0 Hz, 1H), 6.69 (d, J=7.2 Hz, 1H), 6.51 (d, J=8.0 Hz, 1H), 3.88 (s, 2H), 2.27 (s, 3H), 2.18 (s, 3H), 2.10 (s, 3H). MS (ESI) m/z=320.0 [M+H]⁺.

### Example 244. 8-((3-((4,5-Dimethylthiazol-2-yl)carbamoyl)-2-methylphenyl)amino)octanoic acid (BL1-120)

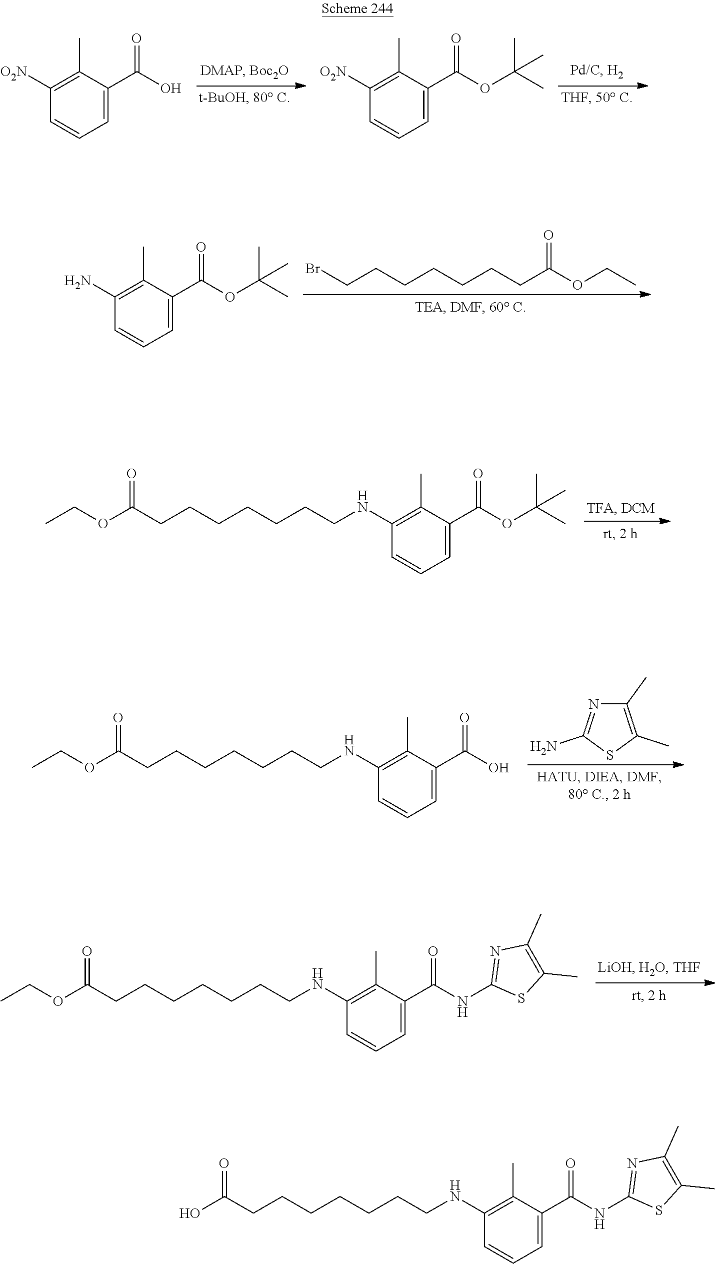

### Step 1. Synthesis of tert-butyl 2-methyl-3-nitrobenzoate

A solution of 2-methyl-3-nitrobenzoic acid (5.0 g, 27.6 mmol), Boc₂O (12.0 g, 55.2 mmol) and DMAP (337 mg, 2.76 mmol) in tert-butanol (30 mL) was stirred at 80° C. for 1 h. Upon completion, the mixture was concentrated in vacuo. The residue was purified by silica gel chromatography (petroleum ether/EtOAc=50:1) to provide the title compound (4.0 g, 61% yield) as a yellow liquid.

### Step 2. Synthesis of tert-butyl 3-amino-2-methylbenzoate

A solution of tert-butyl 2-methyl-3-nitrobenzoate (4.0 g, 16.9 mmol) and Pd/C (10%, 400 mg) in THF (20 mL) was heated at 50° C. overnight under H₂. Upon completion, the mixture was filtered, and the filtrate was concentrated in vacuo. The residue was purified by silica gel chromatography (petroleum ether/EtOAc=20:1) to provide the title compound (3.0 g, 86% yield) as a colorless oil. MS (ESI) m/z=208.2 [M+H]⁺.

### Step 3. Synthesis of tert-butyl 3-((8-ethoxy-8-oxooctyl)amino)-2-methylbenzoate

A solution of tert-butyl 3-amino-2-methylbenzoate (1.00 g, 4.83 mmol), ethyl 8-bromooctanoate (2.42 g, 9.66 mmol), triethylamine (1.47 g, 14.5 mmol) in DMF (10 mL) was stirred at 60° C. overnight. Upon completion, the mixture was diluted with water (100 mL) and extracted with EtOAc (50 mL×3). The combined organic phase was washed with brine (100 mL×2), dried over Na₂SO₄, filtered and concentrated in vacuo. The residue was purified by silica gel chromatography (petroleum ether/EtOAc=3:1) to provide the title compound (700 mg, 39% yield) as a yellow liquid. MS (ESI) m/z=378.2 [M+H]⁺.

### Step 4. Synthesis of 3-((8-ethoxy-8-oxooctyl)amino)-2-methylbenzoic acid

A solution of tert-butyl 3-((8-ethoxy-8-oxooctyl)amino)-2-methylbenzoate (200 mg, 0.531 mmol) in TFA (2 mL) and DCM (2 mL) was stirred at rt for 2 h. Then the mixture was concentrated in vacuo to provide the title compound (150 mg, crude) as a brown oil, which was used for next step directly. MS (ESI) m/z=322.1 [M+H]⁺.

### Step 5. Synthesis of ethyl 8-((3-((4,5-dimethylthiazol-2-yl)carbamoyl)-2-methylphenyl)amino)octanoate

To a solution of 3-((8-ethoxy-8-oxooctyl)amino)-2-methylbenzoic acid (150 mg, crude), 4,5-dimethylthiazol-2-amine (89.7 mg, 0.701 mmol) and HATU (266 mg, 0.701 mmol) in DMF (5 mL) at 80° C. was added DIEA (181 mg, 1.40 mmol). The reaction mixture was stirred at 80° C. for 2 h. Upon completion, the mixture was diluted with water (100 mL) and extracted with EtOAc (100 mL×3). The combined organic phase was washed with brine (100 mL×2), dried over Na₂SO₄, filtered and concentrated in vacuo. The residue was purified by silica gel chromatography (petroleum ether/EtOAc=1:1) to provide the title compound (90 mg, 39% yield) as a white solid. MS (ESI) m/z=432.3 [M+H]⁺.

### Step 6. Synthesis of 8-((3-((4,5-dimethylthiazol-2-yl)carbamoyl)-2-methylphenyl)amino)octanoic acid

A solution of ethyl 8-((3-((4,5-dimethylthiazol-2-yl)carbamoyl)-2-methylphenyl)amino)octanoate (90 mg, 0.209 mmol) and LiOH·H₂O (44 mg, 1.04 mmol) in THF (5 mL) and H₂O (1 mL) was stirred at rt for 2 h. Upon completion, the mixture was diluted with water (20 mL) and acidified to pH=4 with aq. HCl solution (1 M). The mixture was extracted with EtOAc (20 mL×3). The combined organic phase was washed with brine (20 mL×2), dried over Na₂SO₄, filtered and concentrated in vacuo to provide the title compound (35.4 mg, 41% yield) as a white solid. ¹HNMR (400 MHz, DMSO-d₆) δ 12.02 (brs, 1H), 11.9 (brs, 1H), 7.09 (t, J=7.8 Hz, 1H), 6.64 (t, J=7.2 Hz, 2H), 4.99 (t, J=5.6 Hz, 1H)), 3.11-3.08 (m, 2H), 2.27 (s, 3H), 2.21 (t, J=7.2 Hz, 2H), 2.17 (s, 3H), 2.05 (s, 3H), 1.60-1.49 (m, 4H), 1.31-1.26 (m, 6H). MS (ESI) m/z=404.4 [M+H]⁺.

### Example 245. 1-((3-((4,5-Dimethylthiazol-2-yl)carbamoyl)-2-methylphenyl)amino)-3,6,9,12,15-pentaoxaoctadecan-18-oic acid (BL1-121)

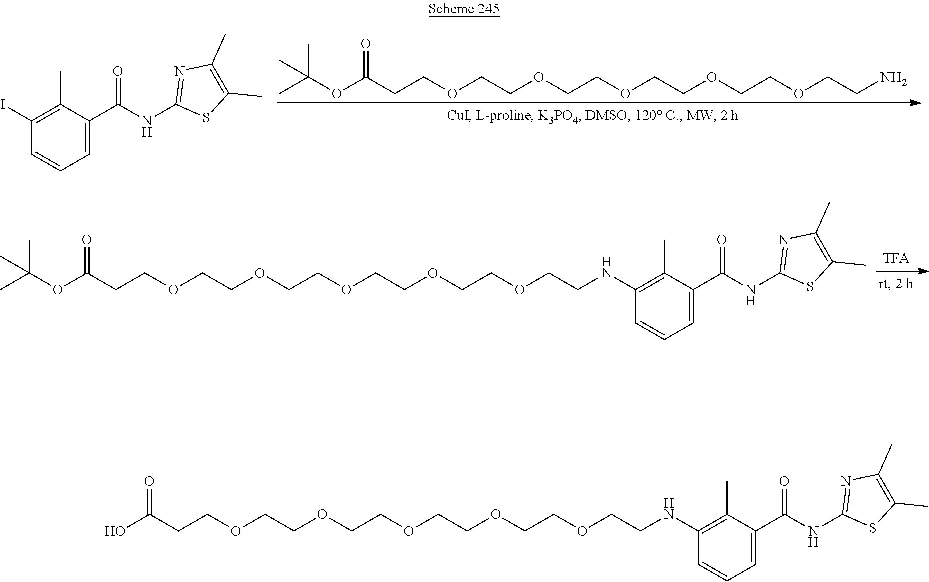

BL1-121 was synthesized following the standard procedure for preparing BL1-105 (TFA salt, 36 mg, 3% yield over 2 steps) as a yellow solid. ¹HNMR (400 MHz, DMSO-d₆) δ 12.09 (brs, 1H), 7.10 (t, J=8.0 Hz, 1H), 6.73-6.67 (m, 2H), 3.63-3.48 (m, 20H), 3.29 (t, J=6.2 Hz, 2H), 2.44 (t, J=6.4 Hz, 2H), 2.27 (s, 3H), 2.17 (s, 3H), 2.06 (s, 3H). MS (ESI) m/z=554.2 [M+H]⁺.

### Example 246. 5-((2-(2-(2-(2-Aminoethoxy)ethoxy)ethoxy)ethyl)amino)-N-(4,5-dimethylthiazol-2-yl)-2-methylbenzamide (BL1-122)

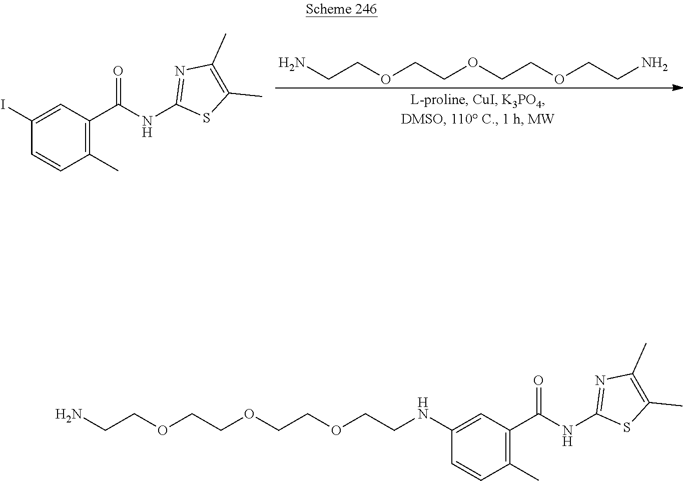 [SMILES: Cc1ccc(I)cc1C(=O)Nc1nc(C)c(C)s1 ; NCCOCCOCCOCCN ; Cc1ccc(NCCOCCOCCOCCN)cc1C(=O)Nc1nc(C)c(C)s1]

BL1-122 was synthesized following the standard procedure for preparing BL1-88 (TFA salt, 340 mg, 58% yield) as a yellow solid. ¹HNMR (400 MHz, DMSO-d₆) δ 12.11 (brs, 1H), 7.79 (brs, 3H), 7.02 (d, J=8.4 Hz, 1H), 6.77 (d, J=2.4 Hz, 1H), 6.70 (dd, J=8.4, 2.4 Hz, 1H), 3.59-3.56 (m, 12H), 3.24 (t, J=5.6 Hz, 2H), 2.98-2.94 (m, 2H), 2.26 (s, 3H), 2.22 (s, 3H), 2.18 (s, 3H). MS (ESI) m/z=437.2 [M+H]⁺.

### Example 247. 3-(2-(3-((2-((4,5-Dimethylthiazol-2-yl)carbamoyl)phenyl)amino)-3-oxopropoxy)ethoxy)propanoic acid (BL1-123)

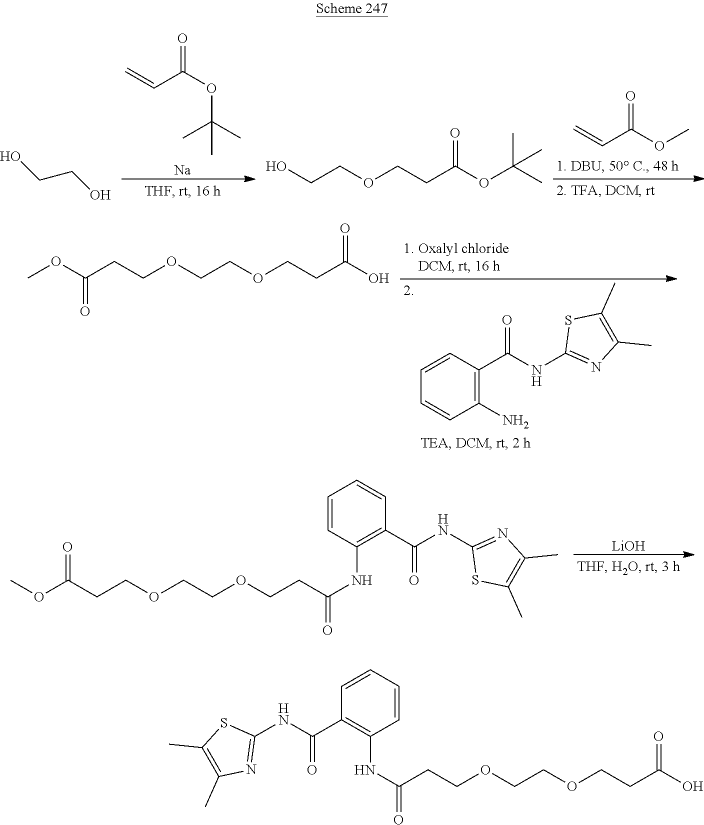

### Step 1. Synthesis of tert-butyl 3-(2-hydroxyethoxy)propanoate

To a solution of ethylene glycol (50 g, 0.8 mol) in THF (300 mL) was added sodium (300 mg, 13 mmol). The mixture was stirred at rt for 2 h. Tert-Butyl acrylate (34.5 g, 0.27 mol) was added. The mixture was stirred at rt for 16 h. Upon completion, the mixture was concentrated, and the residue was purified by silica gel chromatography (EtOAc/petroleum ether=0:1 to 1:1) to provide the title compound (15 g, 29% yield) as a colorless oil.

### Step 2. Synthesis of tert-butyl 3-(2-(3-methoxy-3-oxopropoxy)ethoxy)propanoate

A mixture of tert-butyl 3-(2-hydroxyethoxy)propanoate (8.0 g, 42 mmol), methyl acrylate (16 mL, 177 mmol) and DBU (12.8 g, 84 mmol) was stirred at 50° C. for 48 h. The resulted mixture was concentrated, and the residue was purified by silica gel chromatography (EtOAc/petroleum ether=0:1 to 3:7) to provide the title compound (5.0 g, 52% yield) as a colorless oil.

### Step 3. Synthesis of 3-(2-(3-methoxy-3-oxopropoxy)ethoxy)propanoic acid

To a solution of tert-butyl 3-(2-(3-methoxy-3-oxopropoxy)ethoxy)propanoate (3.0 g, 10.9 mmol) in DCM (10 mL) was added TFA (2 mL). The mixture was stirred at rt for 6 h. TLC showed the reaction was completed. The mixture was concentrated to afford the title compound (2.5 g, 92% yield) as a colorless oil.

### Step 4. Synthesis of methyl 3-(2-(3-chloro-3-oxopropoxy)ethoxy)propanoate

To a solution of 3-(2-(3-methoxy-3-oxopropoxy)ethoxy)propanoic acid (2.0 g, 9.1 mmol) in DCM (5 mL) was added oxalyl chloride (1.38 g, 10.9 mmol) and DMF (2 drops). The mixture was stirred at rt for 16 h. The mixture was concentrated to provide the title compound (2.0 g, 93% yield) as a yellow oil.

### Step 5. Synthesis of methyl 3-(2-(3-((2-((4,5-dimethylthiazol-2-yl)carbamoyl)phenyl)amino)-3-oxopropoxy)ethoxy)propanoate

To a solution of 2-amino-N-(4,5-dimethylthiazol-2-yl)benzamide (400 mg, 1.6 mmol) in DCM (10 mL) was added DIEA (418 mg, 3.2 mmol) and methyl 3-(2-(3-chloro-3-oxopropoxy)ethoxy)propanoate (770 mg, 3.2 mmol). The mixture was stirred at rt for 3 h. Upon completion, the mixture was concentrated, and the residue was purified by reverse-phase chromatography (0.1% TFA in H₂O and ACN) to afford the title compound (300 mg, 41% yield) as a yellow oil. MS (ESI) m/z=450.1 [M+H]⁺.

### Step 6. Synthesis of 3-(2-(3-((2-((4,5-dimethylthiazol-2-yl)carbamoyl)phenyl)amino)-3-oxopropoxy)ethoxy)propanoic acid

To a solution of methyl 3-(2-(3-((2-((4,5-dimethylthiazol-2-yl)carbamoyl)phenyl)amino)-3-oxopropoxy)ethoxy)propanoate (300 mg, 0.57 mmol) in THF (8 mL) and H₂O (2 mL) was added LiOH (140 mg, 3.34 mmol). The mixture was stirred at rt for 3 h. LCMS showed the reaction was completed. The pH was adjusted to 1-2 and extracted with EtOAc. The organic layer was concentrated to provide the title compound (250 mg, 86% yield) as a white solid. MS (ESI) m/z=436.1 [M+H]⁺.

### Example 248. 6-((3-((4,5-Dimethylthiazol-2-yl)carbamoyl)-4-methylphenyl)amino)hexanoic acid (BL1-124)

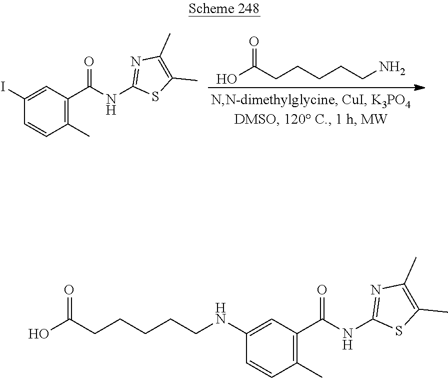

BL1-124 was synthesized following the standard procedure for preparing BL1-103 (TFA salt, 142 mg, 20% yield) as a yellow solid. ¹HNMR (400 MHz, DMSO-d₆) δ 12.17 (brs, 1H), 7.08 (d, J=7.6 Hz, 1H), 6.85-6.80 (m, 2H), 3.07 (d, J=6.4 Hz, 2H), 2.27-2.08 (m, 11H), 1.58-1.51 (m, 4H), 1.41-1.35 (m, 2H). MS (ESI) m/z=376.2 [M+H]⁺.

### Example 249. 3-(2-(2-((3-((4,5-Dimethylthiazol-2-yl)carbamoyl)-4-methylphenyl)amino)ethoxy)ethoxy)propanoic acid (BL1-125)

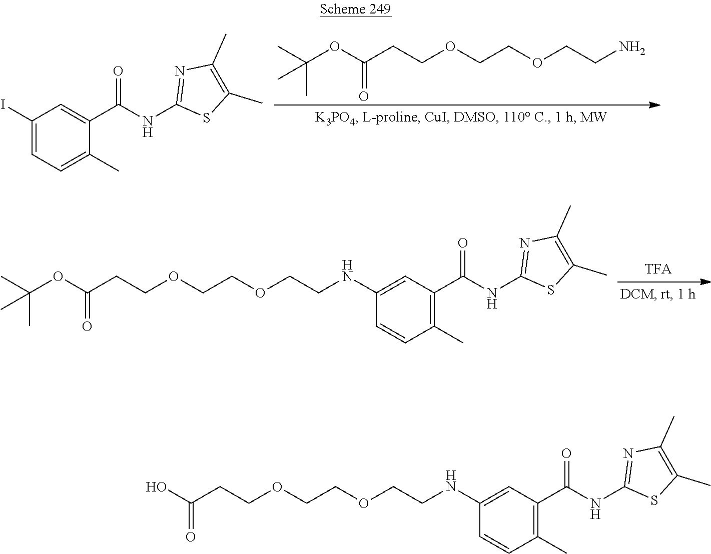

BL1-125 was synthesized following the standard procedure for preparing BL1-105 (TFA salt, 155 mg, 30% yield over 2 steps) as a yellow solid. ¹HNMR (400 MHz, DMSO-d₆) δ 12.31 (brs, 1H), 7.06 (d, J=8.4 Hz, 1H), 6.85 (s, 1H), 6.80-6.78 (m, 1H), 3.63-3.51 (m, 8H), 3.26 (d, J=6.4 Hz, 2H), 2.45 (d, J=6.0 Hz, 2H), 2.27 (s, 3H), 2.25 (s, 3H), 2.19 (s, 3H). MS (ESI) m/z=422.1 [M+H]⁺.

### Example 250. 1-((3-((4,5-Dimethylthiazol-2-yl)carbamoyl)-4-methylphenyl)amino)-3,6,9,12,15,18-hexaoxahenicosan-21-oic acid (BL1-126)

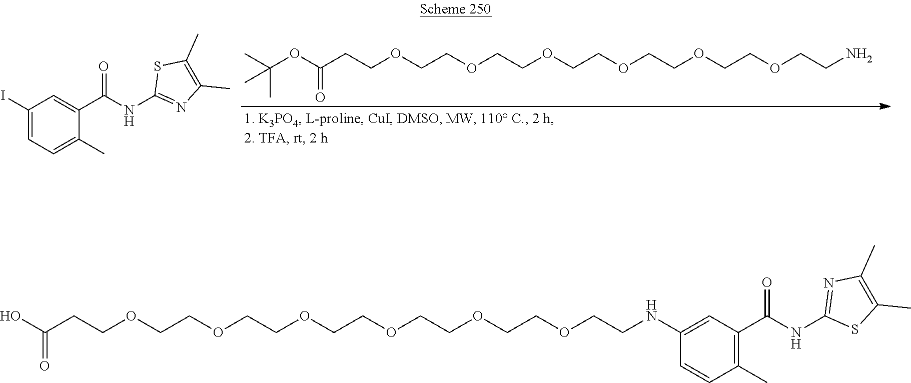

BL1-126 was synthesized following the standard procedure for preparing BL1-105 (TFA salt, 45.0 mg, 8% yield over 2 steps) as a yellow solid. ¹HNMR (400 MHz, DMSO-d₆) δ 12.06 (brs, 1H), 7.02 (d, J=8.4 Hz, 1H), 6.78 (s, 1H), 6.74-6.72 (m, 1H), 3.69-3.51 (m, 24H), 3.25 (t, J=5.8 Hz, 2H), 2.44 (t, J=6.4 Hz, 2H), 2.27 (s, 3H), 2.23 (s, 3H), 2.18 (s, 3H). MS (ESI) m/z=598.2 [M+H]⁺.

### Example 251. 5-((20-Amino-3,6,9,12,15,18-hexaoxaicosyl)amino)-N-(4,5-dimethylthiazol-2-yl)-2-methylbenzamide (BL1-127)

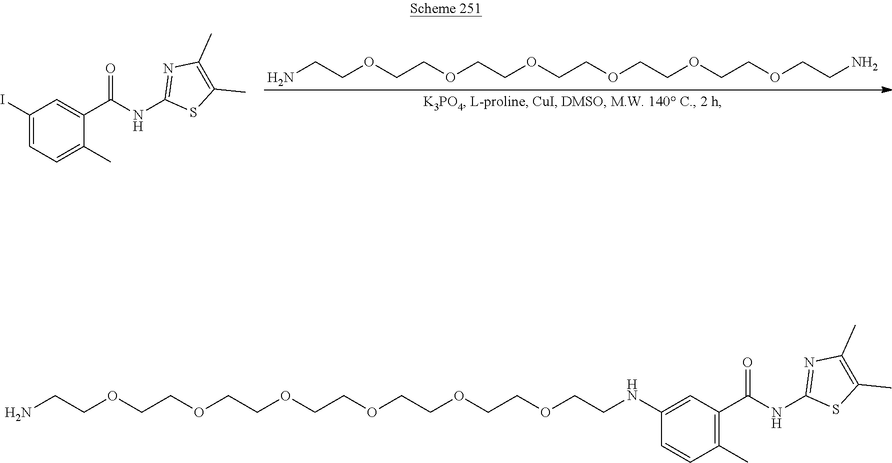 [SMILES: Cc1ccc(I)cc1C(=O)Nc1nc(C)c(C)s1 ; NCCOCCOCCOCCOCCOCCOCCN ; Cc1ccc(NCCOCCOCCOCCOCCOCCOCCN)cc1C(=O)Nc1nc(C)c(C)s1]

BL1-127 was synthesized following the standard procedure for preparing BL1-88 (TFA salt, 110 mg, 40% yield) as a yellow oil. ¹HNMR (400 MHz, DMSO-d₆) δ 7.05 (d, J=8.0 Hz, 1H), 6.78 (d, J=2.4 Hz, 1H), 6.75-6.72 (m, 1H), 3.59-3.51 (m, 24H), 3.24 (t, J=5.6 Hz, 2H), 2.97 (t, J=4.8 Hz, 2H), 2.27 (s, 3H), 2.23 (s, 3H), 2.18 (s, 3H). MS (ESI) m/z=569.3 [M+H]⁺.

### Example 252. 1-((3-((4,5-Dimethylthiazol-2-yl)carbamoyl)-2-methylphenyl)amino)-3,6,9,12,15,18-hexaoxahenicosan-21-oic acid (BL1-128)

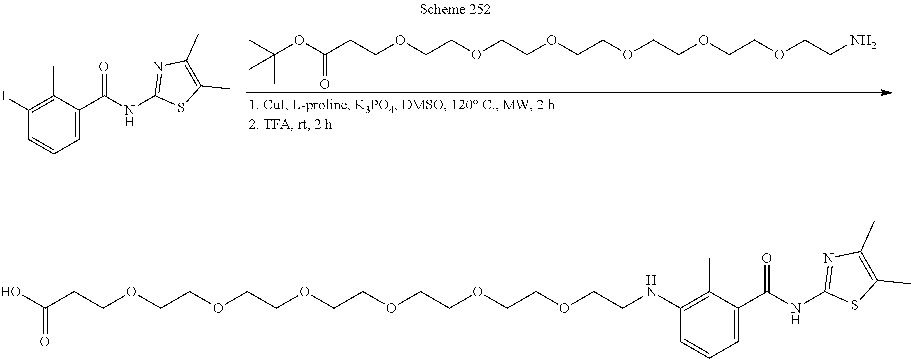

BL1-128 was synthesized following the standard procedure for preparing BL1-105 (26.5 mg, 3% yield over 2 steps) as a yellow solid. ¹HNMR (400 MHz, DMSO-d₆) δ 12.08 (brs, 1H), 7.10 (t, J=7.8 Hz, 1H), 6.73-6.67 (m, 2H), 4.96 (brs, 1H), 3.63-3.49 (m, 24H), 3.27-3.25 (m, 2H), 2.42 (t, J=6.4 Hz, 2H), 2.27 (s, 3H), 2.17 (s, 3H), 2.06 (s, 3H). MS (ESI) m/z=598.2 [M+H]⁺.

### Example 253. 1-((3-((4,5-Dimethylthiazol-2-yl)carbamoyl)-4-methylphenyl)amino)-3,6,9,12,15-pentaoxaoctadecan-18-oic acid (BL1-129)

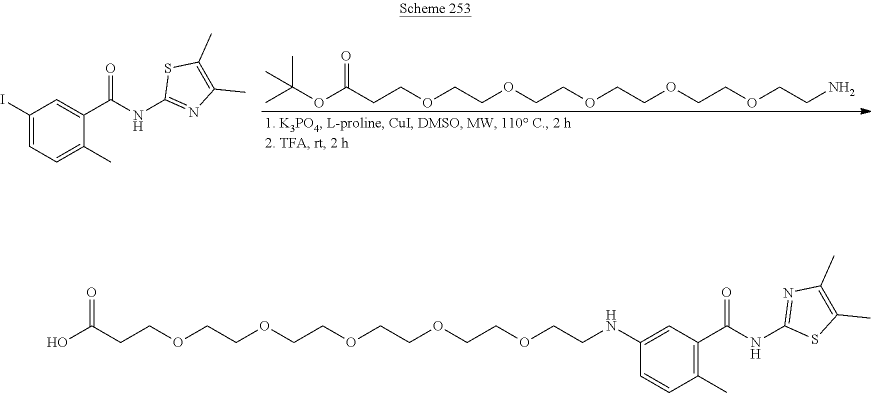

BL1-129 was synthesized following the standard procedure for preparing BL1-105 (TFA salt, 48.8 mg, 9% yield over 2 steps) as a yellow solid. ¹HNMR (400 MHz, DMSO-d₆) δ 12.11 (brs, 1H), 7.04 (d, J=8.4 Hz, 1H), 6.82 (s, 1H), 6.78-6.74 (m, 1H), 3.61-3.49 (m, 20H), 3.26 (t, J=5.6 Hz, 2H), 2.44 (t, J=6.4 Hz, 2H), 2.27 (s, 3H), 2.23 (s, 3H), 2.18 (s, 3H). MS (ESI) m/z=554.2 [M+H]⁺.

### Example 254. (3-((4,5-Dimethylthiazol-2-yl)carbamoyl)-4-methylphenyl)glycine (BL1-130)

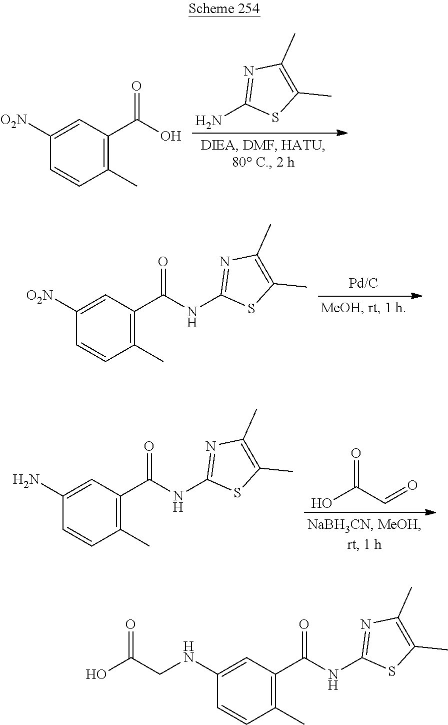

### Step 1. Synthesis of N-(4,5-dimethylthiazol-2-yl)-2-methyl-5-nitrobenzamide

To a solution of 4 2-methyl-5-nitrobenzoic acid (1.00 g, 5.52 mmol), 4,5-dimethylthiazol-2-amine (707 mg, 5.52 mmol) and HATU (2.31 g, 6.07 mmol) in DMF (10 mL) at 80° C. was added DIEA (1.42 g, 11.1 mmol). The mixture was stirred at 80° C. for 2 h. Upon completion, water (50 mL) was added. The mixture was extracted with EtOAc (50 mL×3). The combined organic phase was washed with brine, dried over Na₂SO₄, filtered and concentrated in vacuo. The residue was purified by silica gel chromatography (petroleum ether/EtOAc=1:1) to provide the title compound (500 mg, 31% yield) as a yellow solid. MS (ESI) m/z=292.1 [M+H]⁺.

### Step 2. Synthesis of 5-amino-N-(4,5-dimethylthiazol-2-yl)-2-methylbenzamide

A solution of N-(4,5-dimethylthiazol-2-yl)-2-methyl-5-nitrobenzamide (500 mg. 1.72 mmol) and Pd/C (10%, 100 mg) in MeOH (10 mL) was stirred at rt overnight under H₂. Upon completion, the mixture was filtered, and the filtration was concentrated. The residue was purified by silica gel chromatography (petroleum ether/EtOAc=1:1) to provide the title compound (400 mg, 89% yield) as a white solid, which was used for next step directly. MS (ESI) m/z=262.1 [M+H]⁺.

### Step 3. Synthesis of 3-((4,5-dimethylthiazol-2-yl)carbamoyl)-4-methylphenyl)glycine

A solution of 5-amino-N-(4,5-dimethylthiazol-2-yl)-2-methylbenzamide (400 mg, 1.53 mmol), glyoxylic acid (226 mg, 3.06 mmol) and sodium cyanoborohydride (193 mg, 3.06 mmol) in MeOH (10 mL) was stirred at rt for 1 h. Upon completion, the reaction mixture was filtered. The filter cake was washed with MeOH (20 mL), and dried under reduced pressure to provide the title compound (202 mg, 41% yield) as a white solid. ¹H NMR (400 MHz, DMSO-d₆) δ 12.10 (brs, 1H), 7.00 (d, J=4.4 Hz, 1H), 6.70 (d, J=2.8 Hz, 1H), 6.64 (dd, J=8.0, 2.4 Hz, 1H), 3.84 (s, 2H), 2.26 (s, 3H), 2.22 (s, 3H), 2.17 (s, 3H). MS (ESI) m/z=320.1 [M+H]⁺.

### Example 255. 2-(8-Aminooctanamido)-N-(4,5-dimethylthiazol-2-yl)benzamide (BL1-131)

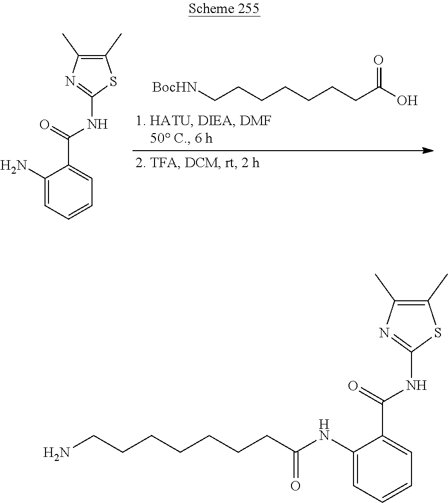 [SMILES: Cc1nc(NC(=O)c2ccccc2N)sc1C ; CC(C)(C)OC(=O)NCCCCCCCC(=O)O ; Cc1nc(NC(=O)c2ccccc2NC(=O)CCCCCCCN)sc1C]

BL1-131 was synthesized following the standard procedure for preparing BL1-144 (204 mg, 83% yield over 2 steps) as a white solid. MS (ESI) m/z=389.0 [M+H]⁺.

### Example 256. 6-((3-((4,5-Dimethylthiazol-2-yl)carbamoyl)-2-methylphenyl)amino)hexanoic acid (BL1-132)

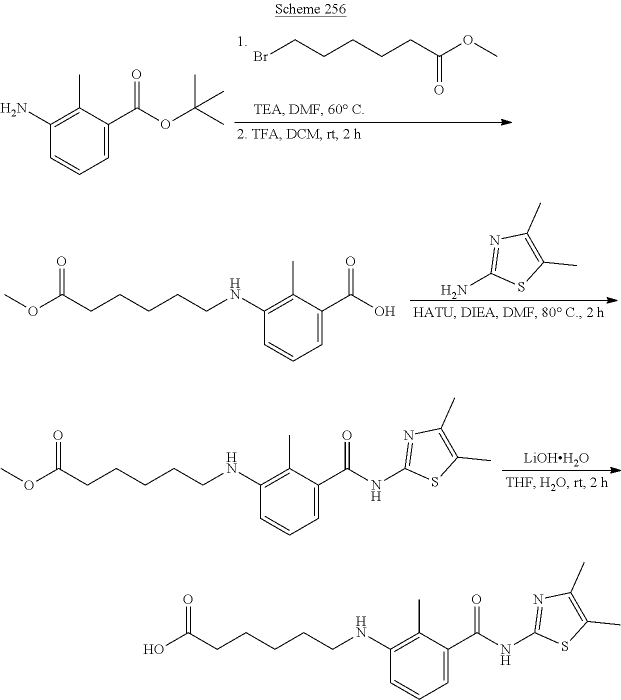

BL1-132 was synthesized following the standard procedure for preparing BL1-120 (100 mg, 16% yield over 4 steps) as a brown solid. ¹HNMR (400 MHz, DMSO-d₆) δ 12.00 (brs, 2H), 7.09 (t, J=7.8 Hz, 1H), 6.64 (t, J=6.8 Hz, 2H), 5.02-5.01 (m, 1H), 3.11-3.08 (m, 2H), 2.27 (s, 3H), 2.23 (t, J=7.2 Hz, 2H), 2.17 (s, 3H), 2.06 (s, 3H), 1.63-1.52 (m, 4H), 1.41-1.34 (m, 2H). MS (ESI) m/z=376.1 [M+H]⁺.

### Example 257. 7-((3-((4,5-Dimethylthiazol-2-yl)carbamoyl)-2-methylphenyl)amino)heptanoic acid (BL1-133)

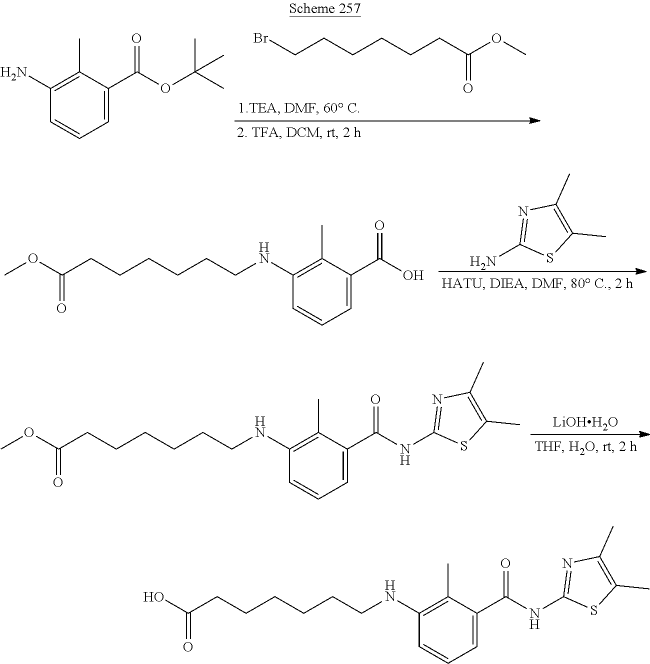

BL1-133 was synthesized following the standard procedure for preparing BL1-120 (109 mg, 6% yield over 4 steps) as a brown solid. ¹HNMR (400 MHz, DMSO-d₆) δ 11.99 (brs, 2H), 7.09 (t, J=7.8 Hz, 1H), 6.64 (t, J=7.0 Hz, 2H), 5.02-5.00 (m, 1H), 3.12-3.07 (m, 2H), 2.27 (s, 3H), 2.22 (t, J=7.2 Hz, 2H), 2.17 (s, 3H), 2.05 (s, 3H), 1.61-1.49 (m, 4H), 1.37-1.32 (m, 4H). MS (ESI) m/z=390.1 [M+H]⁺.

### Example 258. 2-(3-(2-(2-Aminoethoxy)ethoxy)propanamido)-N-(4,5-dimethylthiazol-2-yl)-6-methylbenzamide (BL1-134)

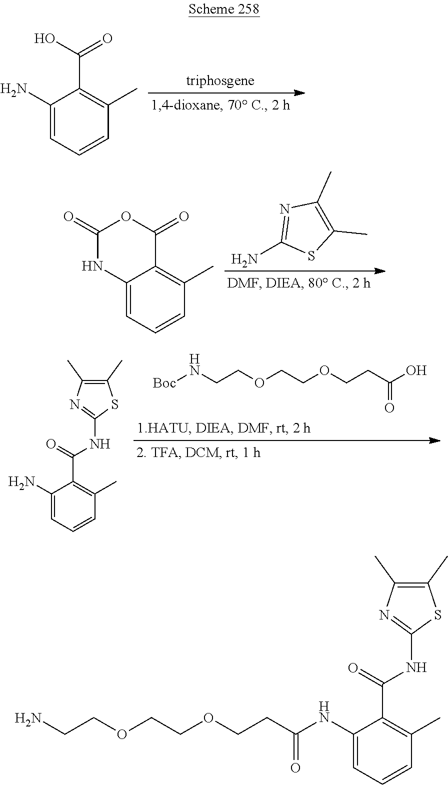

### Step 1. Synthesis of 5-methyl-2H-benzo[d][1,3]oxazine-2,4(1H)-dione

A solution of 2-amino-6-methylbenzoic acid (5.00 g, 33.1 mmol) and triphosgene (3.28 g, 11.3 mmol) in 1,4-dioxane (50 mL) was stirred at 70° C. for 2 h. After cooled to rt, the mixture was filtered, and the cake was dried in vacuo to provide the title compound (3.40 g, 58% yield) as a white solid. MS (ESI) m/z=178.1 [M+H]⁺.

### Step 2. Synthesis of 2-amino-N-(4,5-dimethylthiazol-2-yl)-6-methylbenzamide

A solution of 5-methyl-2H-benzo[d][1,3]oxazine-2,4(1H)-dione (3.40 g, 19.1 mmol), 4,5-dimethylthiazol-2-amine (3.67 g, 28.6 mmol) and DIEA (6.16 g, 47.8 mmol) in DMF (30 mL) was stirred at 80° C. for 2 h. After cooled to rt, the mixture was diluted with water (50 mL) and extracted with EtOAc (50 mL×3). The organic phase was washed with brine, dried over Na₂SO₄, filtered and concentrated. The residue was purified by silica gel chromatography (petroleum ether/EtOAc=2:1) to provide the title compound (1.60 g, 32% yield) as a brown solid. MS (ESI) m/z=262.1 [M+H]⁺.

### Step 3. Synthesis of tert-butyl (2-(2-(3-((2-((4,5-dimethylthiazol-2-yl)carbamoyl)-3-methylphenyl)amino)-3-oxopropoxy)ethoxy)ethyl)carbamate

A solution of 2-amino-N-(4,5-dimethylthiazol-2-yl)-6-methylbenzamide (400 mg, 1.52 mmol), 2,2-dimethyl-4-oxo-3,8,11-trioxa-5-azatetradecan-14-oic acid (422 mg, 1.52 mmol), HATU (870 mg, 2.29 mmol) and DIEA (592 mg, 4.57 mmol) in DMF (3 mL) was stirred at rt for 2 h. The mixture was then diluted with water (50 mL) and extracted with EtOAc (50 mL×3). The organic phase was washed by brine, dried over Na₂SO₄, filtered and concentrated. The residue was purified by silica gel chromatography (petroleum ether/EtOAc=1:1) to provide the title compound (250 mg, 32% yield) as a yellow solid. MS (ESI) m/z=521.1 [M+H]⁺.

### Step 4. Synthesis of 2-(3-(2-(2-aminoethoxy)ethoxy)propanamido)-N-(4,5-dimethylthiazol-2-yl)-6-methylbenzamide

A solution of tert-butyl (2-(2-(3-((2-((4,5-dimethylthiazol-2-yl)carbamoyl)-3-methylphenyl)amino)-3-oxopropoxy)ethoxy)ethyl)carbamate (200 mg, 0.384 mmol) in DCM (2 mL) and TFA (2 mL) was stirred at rt for 1 h. The reaction mixture was concentrated. The residue was purified by reverse-phase HPLC (0.1% TFA) to provide the title compound (TFA salt, 102.2 mg, 50% yield) as a brown solid. ¹HNMR (400 MHz, DMSO-d₆) δ 12.11 (brs, 1H), 9.39 (s, 1H), 7.76 (brs, 3H), 7.42 (d, J=7.6 Hz, 1H), 7.33 (t, J=7.8 Hz, 1H), 7.11 (d, J=7.2 Hz, 1H), 3.54-3.49 (m, 10H), 2.98-2.94 (m, 2H), 2.28 (s, 3H), 2.26 (s, 3H), 2.18 (s, 3H). MS (ESI) m/z=421.1 [M+H]⁺.

### Example 259. 2-(3-(2-(2-Aminoethoxy)ethoxy)propanamido)-4-chloro-N-(4,5-dimethylthiazol-2-yl)benzamide (BL1-135)

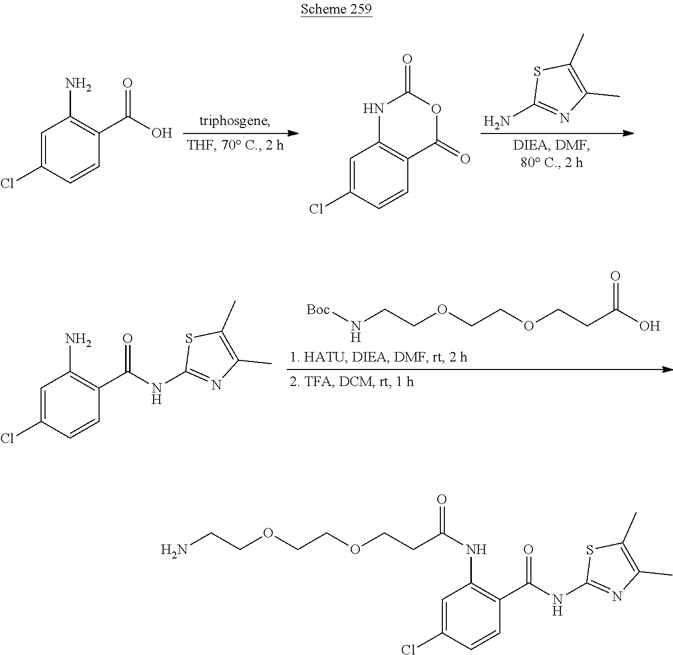

BL1-135 was synthesized following the standard procedure for preparing BL1-134 (TFA salt, 95.2 mg, 3% yield over 4 steps) as a white solid. ¹HNMR (400 MHz, DMSO-d₆) δ 11.73 (brs, 1H), 8.51 (s, 1H), 8.11 (d, J=8.0 Hz, 1H), 7.7 (brs, 3H), 7.23 (dd, J=8.4, 2.0 Hz, 1H), 3.74 (t, J=6.0 Hz, 2H), 3.59-3.53 (m, 6H), 2.94-2.89 (m, 2H), 2.70-2.67 (m, 2H), 2.24 (s, 3H), 2.20 (s, 3H). MS (ESI) m/z=441.13 [M+H]⁺.

### Example 260. 2-(3-(2-(2-Aminoethoxy)ethoxy)propanamido)-N-(4,5-dimethylthiazol-2-yl)-5-methylbenzamide (BL1-136)

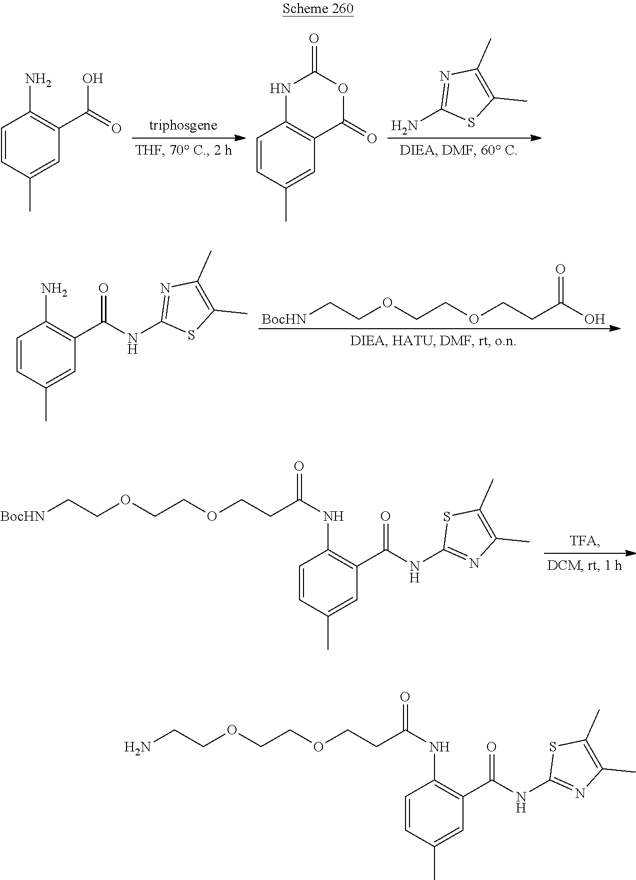

BL1-136 was synthesized following the standard procedure for preparing BL1-134 (TFA salt, 372 mg, 9% yield over 4 steps) as a yellow solid. ¹HNMR (400 MHz, DMSO-d₆) δ 11.03 (brs, 1H), 8.14 (d, J=8.0 Hz, 1H), 7.82-7.78 (m, 4H), 7.32 (dd, J=8.4, 1.6 Hz, 1H), 3.71 (t, J=6.0 Hz, 2H), 3.59-3.54 (m, 6H), 2.94-2.90 (m, 2H), 2.60 (t, J=6.0 Hz, 2H), 2.31 (s, 3H), 2.25 (s, 3H), 2.20 (s, 3H). MS (ESI) m/z=421.1 [M+H]⁺.

### Example 261. 2-(3-(2-(2-Aminoethoxy)ethoxy)propanamido)-5-chloro-N-(4,5-dimethylthiazol-2-yl)benzamide (BL1-137)

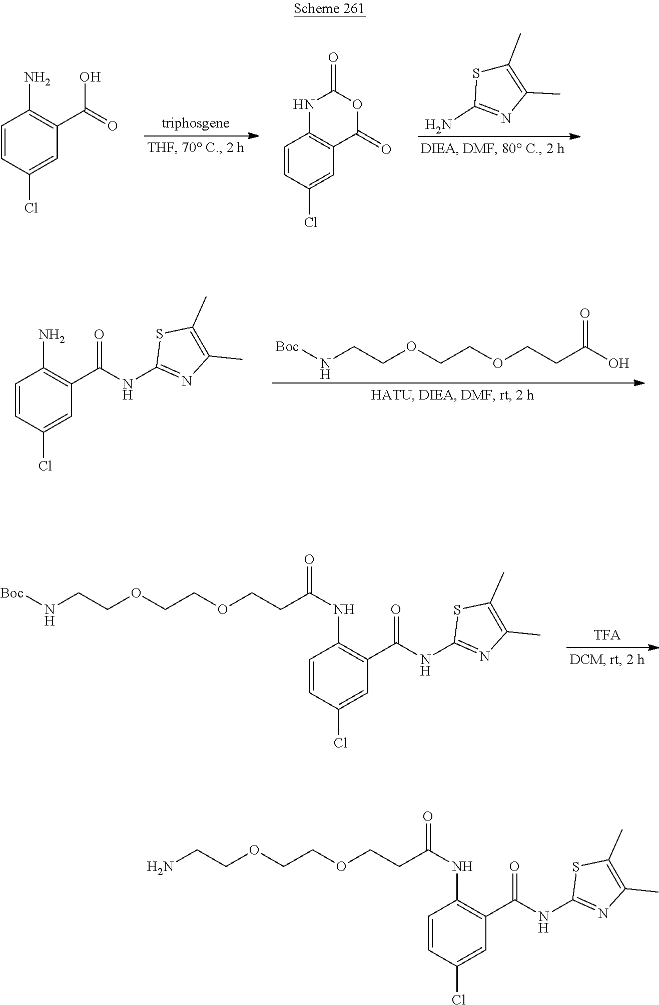

BL1-137 was synthesized following the standard procedure for preparing BL1-134 (TFA salt, 60.1 mg, 5% yield over 4 steps) as a yellow solid. ¹HNMR (400 MHz, DMSO-d₆) δ 8.37 (brs, 1H), 8.11 (brs, 1H), 7.69 (brs, 3H), 7.57-7.54 (m, 1H), 3.74-3.69 (m, 2H), 3.57-3.53 (m, 6H), 2.95-2.90 (m, 2H), 2.67-2.64 (m, 2H), 2.45 (s, 3H), 2.20 (s, 3H). MS (ESI) m/z=441.1 [M+H]⁺.

### Example 262. 2-(3-(2-(2-Aminoethoxy)ethoxy)propanamido)-N-(4,5-dimethylthiazol-2-yl)-4-fluorobenzamide (BL1-138)

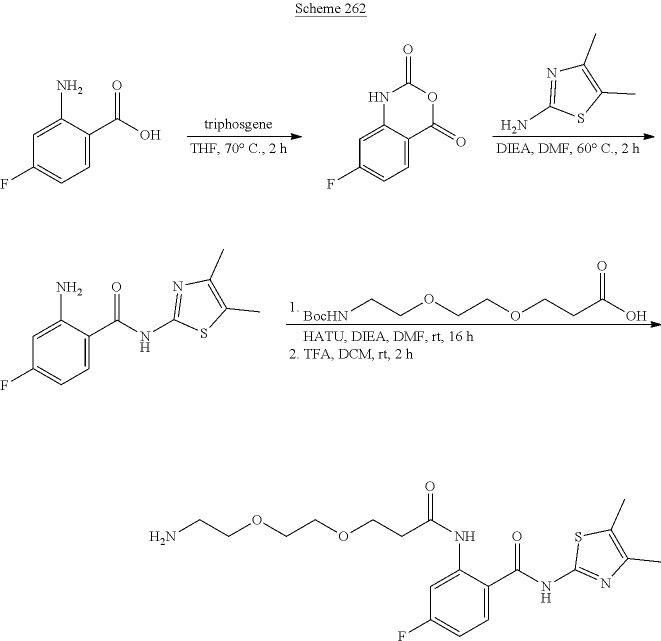

BL1-138 was synthesized following the standard procedure for preparing BL1-134 (TFA salt, 100 mg, 5% yield over 4 steps) as a yellow solid. ¹HNMR (400 MHz, DMSO-d₆) δ 11.8 (s, 1H), 8.27-8.18 (m, 2H), 7.74 (brs, 3H), 7.02-7.01 (m, 1H), 3.76-3.73 (m, 2H), 3.58-3.53 (m, 6H), 2.94-2.90 (m, 2H), 2.70-2.67 (m, 2H), 2.24 (br s, 3H), 2.20 (br s, 3H). MS (ESI) m/z=425.1 [M+H]⁺.

### Example 263. 2-(3-(2-(2-Aminoethoxy)ethoxy)propanamido)-4-bromo-N-(4,5-dimethylthiazol-2-yl)benzamide (BL1-139)

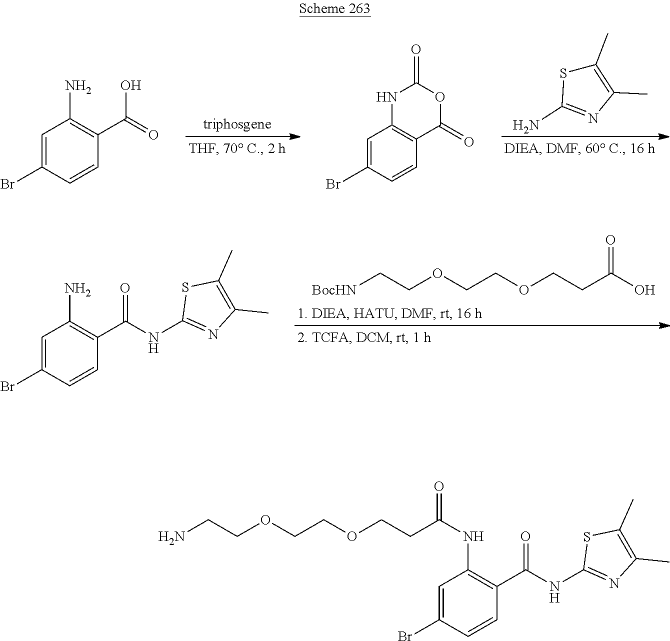

BL1-139 was synthesized following the standard procedure for preparing BL1-134 (TFA salt, 58.6 mg, 2% yield over 4 steps) as a yellow solid. ¹HNMR (400 MHz, DMSO-d₆) δ 8.65 (brs, 1H), 8.04 (brs, 1H), 7.71 (brs, 3H), 7.37 (d, J=8.8 Hz, 1H), 3.74 (t, J=5.6 Hz, 2H), 3.58-3.45 (m, 6H), 2.94-2.92 (m, 2H), 2.69-2.68 (m, 2H), 2.24 (s, 3H), 2.20 (s, 3H). MS (ESI) m/z=485.1/487.1 [M+H]⁺.

### Example 264. 2-(3-(2-(2-Aminoethoxy)ethoxy)propanamido)-5-bromo-N-(4,5-dimethylthiazol-2-yl)benzamide (BL1-140)

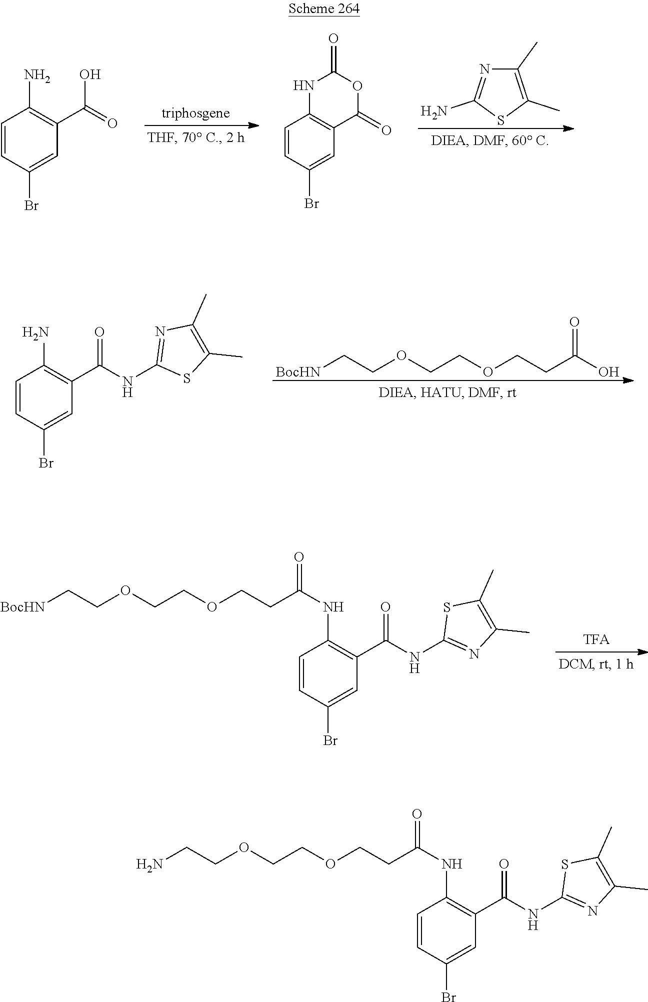

BL1-140 was synthesized following the standard procedure for preparing BL1-134 (TFA salt, 510 mg, 24% yield over 4 steps) as a yellow solid. ¹HNMR (400 MHz, DMSO-d₆) δ 11.61 (brs, 1H), 8.35-8.25 (m, 2H), 7.75 (brs, 3H), 7.69-7.66 (m, 1H), 3.73 (t, J=5.8 Hz, 2H), 3.57-3.53 (m, 6H), 2.94-2.91 (m, 2H), 2.67-2.64 (m, 2H), 2.24 (s, 3H), 2.20 (s, 3H). MS (ESI) m/z=485.0/487.0 [M+H]⁺.

### Example 265. 2-(3-Aminopropanamido)-N-(4,5-dimethylthiazol-2-yl)benzamide (BL1-141)

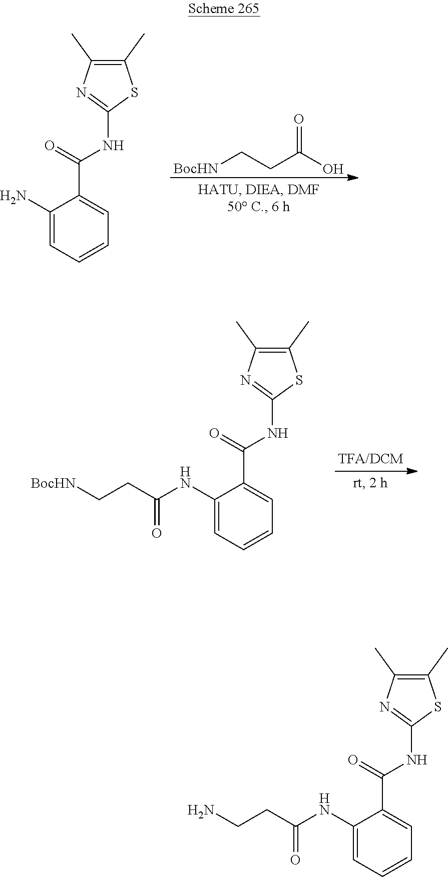 [SMILES: Cc1nc(NC(=O)c2ccccc2N)sc1C ; CC(C)(C)OC(=O)NCCC(=O)O ; Cc1nc(NC(=O)c2ccccc2NC(=O)CCNC(=O)OC(C)(C)C)sc1C ; Cc1nc(NC(=O)c2ccccc2NC(=O)CCN)sc1C]

BL1-141 was synthesized following the standard procedure for preparing BL1-144 (933 mg, 90% yield over 2 steps) as a white solid. MS (ESI) m/z=319.1 [M+H]⁺.

### Example 266. 2-(3-(2-Aminoethoxy)propanamido)-N-(4,5-dimethylthiazol-2-yl)benzamide (BL1-142)

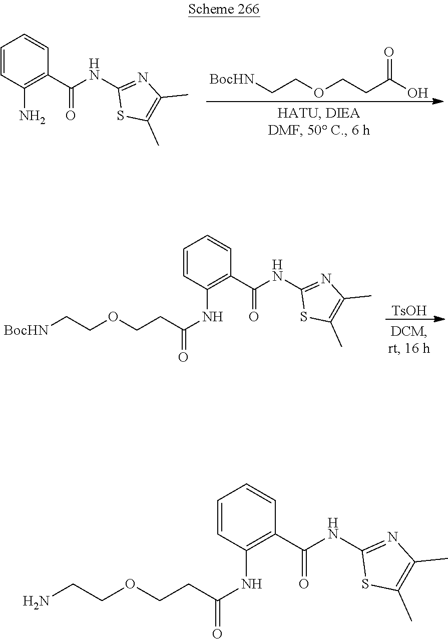 [SMILES: Cc1nc(NC(=O)c2ccccc2N)sc1C ; CC(C)(C)OC(=O)NCCOCCC(=O)O ; Cc1nc(NC(=O)c2ccccc2NC(=O)CCOCCNC(=O)OC(C)(C)C)sc1C ; "TsOH" ; Cc1nc(NC(=O)c2ccccc2NC(=O)CCOCCN)sc1C]

### Step 1. Synthesis of tert-butyl (2-(3-((2-((4,5-dimethylthiazol-2-yl)carbamoyl)phenyl)amino)-3-oxopropoxy)ethyl)carbamate

To a solution of 2-amino-N-(4,5-dimethylthiazol-2-yl)benzamide (400 mg, 1.62 mmol) in DMF (5 mL) were added 3-(2-((tert-butoxycarbonyl)amino)ethoxy)propanoic acid (754 mg, 3.23 mmol), HATU (1.23 g, 3.23 mmol) and DIEA (418 mg, 3.23 mmol). The mixture was stirred at 50° C. for 6 h. Upon completion, the mixture was extracted with EtOAc (20 ml×3). The combined organic layer was washed with brine (30 mL), dried over sodium sulfate, filtered and concentrated to dryness. The residue was purified by reverse-phase chromatography (0.1% TFA in H₂O and ACN) to afford the title compound (550 mg, 74% yield) as a yellow oil. MS (ESI) m/z=463.1 [M+H]⁺.

### Step 2. Synthesis of 2-(3-(2-aminoethoxy)propanamido)-N-(4,5-dimethylthiazol-2-yl)benzamide

To a solution of tert-butyl (2-(3-((2-((4,5-dimethylthiazol-2-yl)carbamoyl)phenyl)amino)-3-oxopropoxy)ethyl)carbamate (550 mg, 1.19 mmol) in DCM (10 mL) was added TsOH (1.02 g, 5.92 mmol). The mixture was stirred at rt for 16 h. Upon completion, the mixture was concentrated and purified by reverse-phase chromatography (0.1% NH₄HCO₃ in H₂O and ACN) to provide the title compound (215 mg, 50% yield) as a yellow solid. MS (ESI) m/z=363.1 [M+H]⁺.

### Example 267. 2-(3-(2-(2-Aminoethoxy)ethoxy)propanamido)-5-(butylamino)-N-(4,5-dimethylthiazol-2-yl)benzamide (BL1-143)

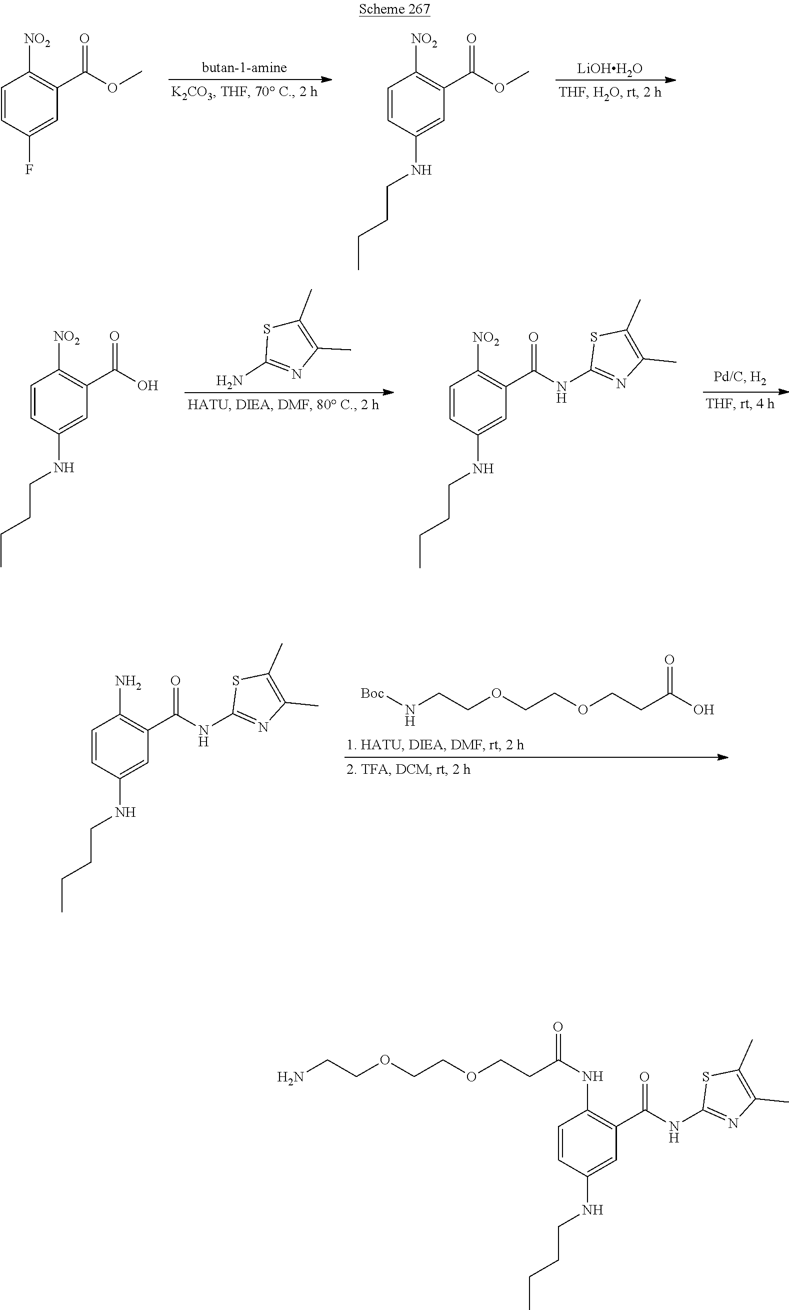

### Step 1. Synthesis of methyl 5-(butylamino)-2-nitrobenzoate

A solution of methyl 5-fluoro-2-nitrobenzoate (2.00 g, 10.1 mmol), butan-1-amine (1.47 g, 20.1 mmol) and K₂CO₃ (2.77 g, 20.1 mmol) in THF (10 mL) was stirred at 70° C. for 2 h. After cooled to rt, the mixture was diluted with water (100 mL) and extracted with EtOAc (50 mL×2). The combined organic phase was washed with brine, dried over Na₂SO₄, filtered and concentrated in vacuo. The residue was purified by silica gel chromatography (petroleum ether/EtOAc=5:1) to provide the title compound (1.60 g, 63% yield) as a yellow solid. MS (ESI) m/z=253.1 [M+H]⁺.

### Step 2. Synthesis of 5-(butylamino)-2-nitrobenzoic acid

A solution of methyl 5-(butylamino)-2-nitrobenzoate (1.60 g, 6.35 mmol) and LiOH·H₂O (1.87 g, 44.6 mmol) in THF and H₂O (20 mL, v/v=4:1) was stirred at rt for 2 h. Water (100 mL) was added. pH of the mixture was adjusted to 4 with HCl (1 M). The obtained mixture was extracted with EtOAc (50 mL×2). The combined organic phase was washed with brine (100 mL×2), dried over Na₂SO₄, filtered and concentrated in vacuo to provide the title compound (1.20 g, 79% yield) as a yellow solid. MS (ESI) m/z=239.1 [M+H]⁺.

### Step 3. Synthesis of 5-(butylamino)-N-(4,5-dimethylthiazol-2-yl)-2-nitrobenzamide

A solution of 5-(butylamino)-2-nitrobenzoic acid (700 mg, 2.94 mmol), 4,5-dimethylthiazol-2-amine (564 mg, 4.41 mmol), HATU (1.34 g, 3.53 mmol) and DIEA (1.14 g, 8.82 mmol) in DMF (10 mL) was stirred at 80° C. for 2 h. After cooled to rt, the mixture was diluted with water (100 mL) and extracted with EtOAc (50 mL×2). The combined organic phase was washed with brine, dried over Na₂SO₄, filtered and concentrated in vacuo. The residue was purified by silica gel chromatography (petroleum ether/EtOAc=3:1) to provide the title compound (600 mg, 59% yield) as a yellow solid. MS (ESI) m/z=349.1 [M+H]⁺.

### Step 4. Synthesis of 2-amino-5-(butylamino)-N-(4,5-dimethylthiazol-2-yl)benzamide

A solution of 5-(butylamino)-N-(4,5-dimethylthiazol-2-yl)-2-nitrobenzamide (600 mg, 1.72 mmol) and Pd/C (10%, 100 mg) in THF (15 mL) was stirred at rt for 4 h under H₂. The mixture was filtered, and the filtrate was concentrated in vacuo. The residue was purified by silica gel chromatography (petroleum ether/EtOAc=2:1) to provide the title compound (500 mg, 91% yield) as colorless oil. MS (ESI) m/z=319.1 [M+H]⁺.

### Step 5. Synthesis of tert-butyl (2-(2-(3-((4-(butylamino)-2-((4,5-dimethylthiazol-2-yl)carbamoyl)phenyl)amino)-3-oxopropoxy)ethoxy)ethyl)carbamate

A solution of 2-amino-5-(butylamino)-N-(4,5-dimethylthiazol-2-yl)benzamide (300 mg, 0.943 mmol), 2,2-dimethyl-4-oxo-3,8,11-trioxa-5-azatetradecan-14-oic acid (287 mg, 1.03 mmol), HATU (783 mg, 2.06 mmol) and DIEA (266 mg, 2.06 mmol) in DMF (3 mL) was stirred at rt for 2 h. The mixture was purified by reverse-phase HPLC (0.1% formic acid in water and ACN) to provide the title compound (150 mg, 28% yield) as a brown solid. MS (ESI) m/z=578.2 [M+H]⁺.

### Step 6. Synthesis of 2-(3-(2-(2-aminoethoxy)ethoxy)propanamido)-5-(butylamino)-N-(4,5-dimethylthiazol-2-yl)benzamide

A solution of tert-butyl (2-(2-(3-((4-(butylamino)-2-((4,5-dimethylthiazol-2-yl)carbamoyl)phenyl)amino)-3-oxopropoxy)ethoxy)ethyl)carbamate (150 mg, 0.260 mmol) in TFA (2 mL) and DCM (2 mL) was stirred at rt for 2 h. Upon completion, the mixture was concentrated in vacuo to provide the title compound (TFA salt, 136 mg, 88% yield) as a yellow oil. ¹HNMR (400 MHz, DMSO-d₆) δ 7.76-7.71 (m, 5H), 7.01 (brs, 1H), 6.83-6.80 (brs, 1H), 3.67 (t, J=4.8 Hz, 2H), 3.57-3.50 (m, 8H), 3.06 (t, J=7.2 Hz, 2H), 2.95-2.91 (m, 2H), 2.26 (s, 3H), 2.19 (s, 3H), 1.59-1.51 (m, 2H), 1.44-1.36 (m, 2H), 0.92 (t, J=7.2 Hz, 3H). MS (ESI) m/z=478.2 [M+H]⁺.

### Example 268. 2-(3-(2-(2-Aminoethoxy)ethoxy)propanamido)-N-(4,5-dimethylthiazol-2-yl)-4-methylbenzamide (BL1-144)

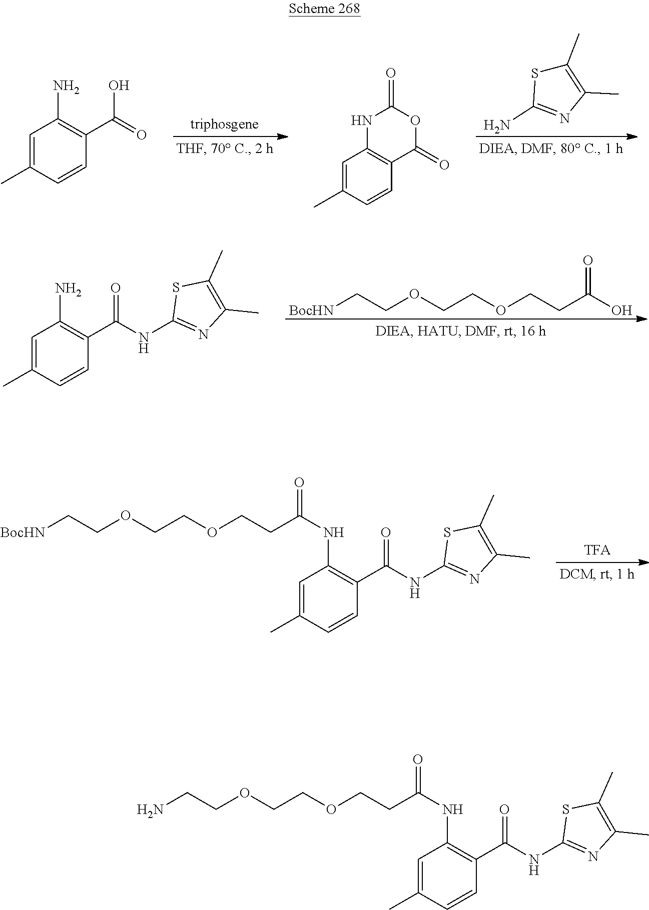

### Step 1. Synthesis of 7-methyl-2H-benzo[d][1,3]oxazine-2,4(1H)-dione

A solution of 2-amino-4-methylbenzoic acid (1.00 g, 6.62 mmol) and triphosgene (647 mg, 2.19 mmol) in THF (10 mL) was heated at 70° C. for 2 h under inert atmosphere. After cooled to rt, the mixture was filtered. The solid was dried under reduced pressure to provide the title compound (1.00 g, 85% yield) as a white solid. MS (ESI) m/z=178.1 [M+H]⁺.

### Step 2. Synthesis of 2-amino-N-(4,5-dimethylthiazol-2-yl)-4-methylbenzamide

A solution of 7-methyl-2H-benzo[d][1,3]oxazine-2,4(1H)-dione (1.00 g, 5.65 mmol), 4,5-dimethylthiazol-2-amine (1.08 g, 8.47 mmol) and DIEA (1.45 g, 11.3 mmol) in DMF (10 mL) was stirred at 80° C. for 1 h. After cooled to rt, the mixture was diluted with EtOAc (50 mL), washed with brine (50 mL×2), dried over Na₂SO₄, filtered and concentrated. The residue was purified by silica gel chromatography (petroleum ether/EtOAc=4:1) to provide the title compound (638 mg, 43% yield) as a yellow solid. MS (ESI) m/z=262.0 [M+H]⁺.

### Step 3. Synthesis of tert-butyl (2-(2-(3-((2-((4,5-dimethylthiazol-2-yl)carbamoyl)-5-methylphenyl)amino)-3-oxopropoxy)ethoxy)ethyl)carbamate

A solution of 2-amino-N-(4,5-dimethylthiazol-2-yl)-4-methylbenzamide (588 mg, 2.25 mmol), 2,2-dimethyl-4-oxo-3,8,11-trioxa-5-azatetradecan-14-oic acid (749 mg, 2.70 mmol), HATU (1.11 g, 2.90 mmol) and DIEA (871 mg, 6.75 mmol) in DMF (6 mL) was stirred at rt for 16 h. Upon completion, the mixture was diluted with water (50 mL) and extracted with EtOAc (50 mL×3). The combined organic phase was washed with brine (50 mL×2), dried over Na₂SO₄, filtered and concentrated in vacuo. The residue was purified by silica gel chromatography (petroleum ether/EtOAc=2:1) to provide the title compound (867 mg, 74% yield) as a yellow solid. MS (ESI) m/z=521.2 [M+H]⁺.

### Step 4. Synthesis of 2-(3-(2-(2-aminoethoxy)ethoxy)propanamido)-N-(4,5-dimethylthiazol-2-yl)-4-methylbenzamide

A solution of (2-(2-(3-((2-((4,5-dimethylthiazol-2-yl)carbamoyl)-5-methylphenyl)amino)-3-oxopropoxy)ethoxy)ethyl)carbamate (867 mg, 1.67 mmol) in TFA (2 mL) and DCM (2 mL) was stirred at rt for 1 h. Upon completion, the mixture was concentrated to provide the title compound (TFA salt, 547 mg, 61% yield) as a yellow solid. ¹HNMR (400 MHz, DMSO-d₆) δ 11.26 (s, 1H), 8.18 (s, 1H), 7.90 (d, J=7.2 Hz, 1H), 7.74 (brs, 3H), 7.90 (dd, J=8.8, 0.8 Hz, 1H), 3.72 (d, J=6.0 Hz, 2H), 3.59-3.54 (m, 6H), 2.92-2.89 (m, 2H), 2.63 (d, J=6.0 Hz, 2H), 2.34 (s, 3H), 2.25 (s, 3H), 2.19 (s, 3H). MS (ESI) m/z=421.1 [M+H]⁺.

### Example 269. 2-(3-(2-(2-Aminoethoxy)ethoxy)propanamido)-N-(4,5-dimethylthiazol-2-yl)-5-(methylamino)benzamide (BL1-145)

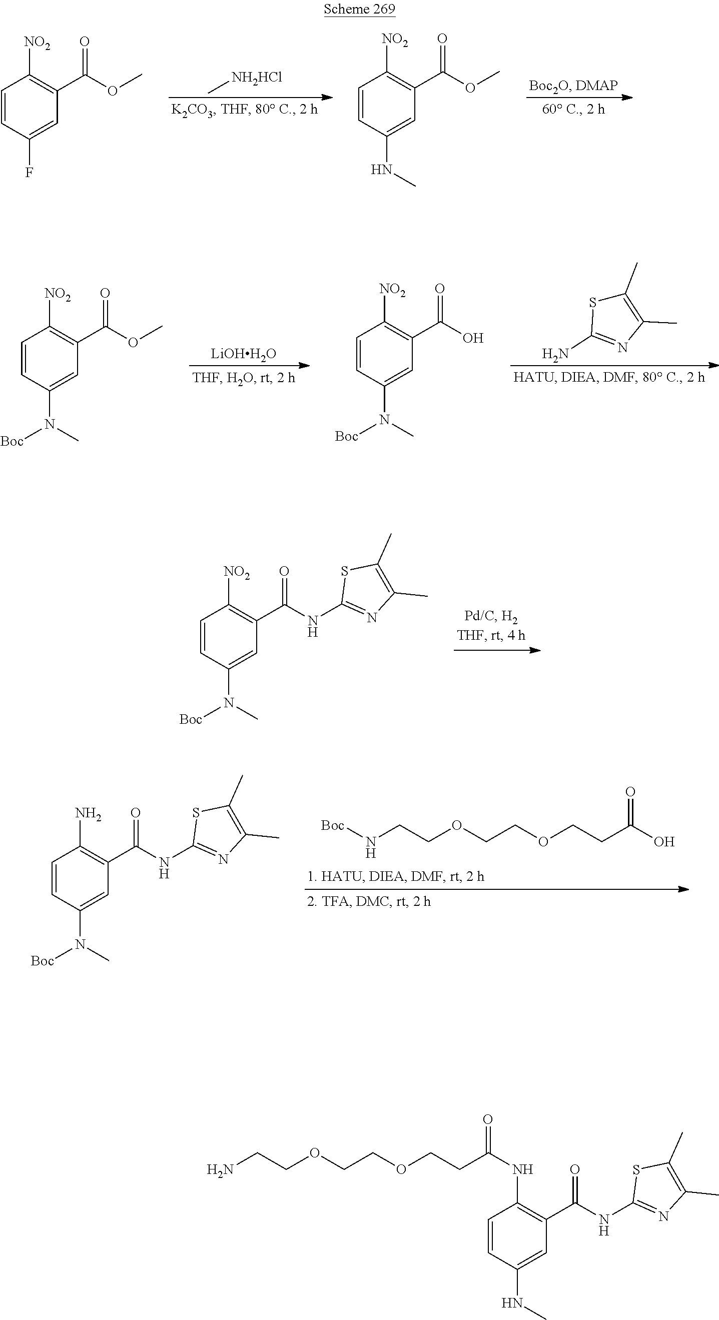

### Step 1. Synthesis of methyl 5-(methylamino)-2-nitrobenzoate

A solution of methyl 5-fluoro-2-nitrobenzoate (2.00 g, 10.1 mmol), methylamine hydrochloride (1.36 g, 20.1 mmol) and K₂CO₃ (2.77 g, 20.1 mmol) in DMF (20 mL) was stirred at 80° C. for 2 h. After cooled to rt, the mixture was diluted with water (100 mL) and extracted with EtOAc (50 mL×2). The combined organic phase was washed with brine, dried over Na₂SO₄, filtered and concentrated in vacuo. The residue was purified by silica gel chromatography (petroleum ether/EtOAc=3:1) to provide the title compound (2.00 g, 94% yield) as a yellow solid. MS (ESI) m/z=211.1 [M+H]⁺.

### Step 2. Synthesis of methyl 5-((tert-butoxycarbonyl)(methyl)amino)-2-nitrobenzoate

A solution of methyl 5-(methylamino)-2-nitrobenzoate (2.00 g, 9.52 mmol) and DMAP (116 mg, 0.952 mmol) in Boc₂O (20 mL) was stirred at 60° C. for 2 h. After cooled to rt, the mixture was concentrated in vacuo. The residue was purified by silica gel chromatography (petroleum ether/EtOAc=10:1) to provide the title compound (1.30 g, 44% yield) as a yellow solid. MS (ESI) m/z=255.0 [M-56+H]⁺.

### Step 3. Synthesis of 5-((tert-butoxycarbonyl)(methyl)amino)-2-nitrobenzoic acid

A solution of methyl 5-((tert-butoxycarbonyl)(methyl)amino)-2-nitrobenzoate (1.30 g, 4.19 mmol) and LiOH—H₂O (881 mg, 21.0 mmol) in THF (10 mL) and H₂O (5 mL) was stirred at rt for 2 h. The mixture was diluted with water (100 mL). pH was adjusted to 4 with aqueous HCl solution (1 M). The mixture was extracted with EtOAc (50 mL×2). The combined organic phase was washed with brine, dried over Na₂SO₄, filtered and concentrated in vacuo to provide the title compound (1.00 g, 81% yield) as a yellow solid. MS (ESI) m/z=295.1 [M−H]—.

### Step 4. Synthesis of tert-butyl (3-((4,5-dimethylthiazol-2-yl)carbamoyl)-4-nitrophenyl)(methyl)carbamate

A solution of 5-((tert-butoxycarbonyl)(methyl)amino)-2-nitrobenzoic acid (1.00 g, 3.38 mmol), 4,5-dimethylthiazol-2-aminein (649 mg, 5.07 mmol), HATU (1.93 g, 5.07 mmol) and DIEA (872 mg, 6.76 mmol) in DMF (10 mL) was stirred at 80° C. for 2 h. After cooled to rt, the mixture was diluted with water (50 mL) and extracted with EtOAc (30 mL×2). The combined organic phase was washed with brine, dried over Na₂SO₄, filtered and concentrated in vacuo. The residue was purified by silica gel chromatography (petroleum ether/EtOAc=5:1) to provide the title compound (600 mg, 44% yield) as a brown solid. MS (ESI) m/z=407.0 [M+H]⁺.

### Step 5. Synthesis of tert-butyl (4-amino-3-((4,5-dimethylthiazol-2-yl)carbamoyl)phenyl)(methyl)carbamate

A solution of tert-butyl (3-((4,5-dimethylthiazol-2-yl)carbamoyl)-4-nitrophenyl)(methyl)carbamate (600 mg, 1.48 mmol) and Pd/C (10%, 100 mg) in THF (6 mL) was stirred at rt for 4 h. Upon completion, the mixture was filtered and the filtrate was concentrated in vacuo. The residue was purified by silica gel chromatography (petroleum ether/EtOAc=5:1) to provide the title compound (500 mg, 90% yield) as a white solid. MS (ESI) m/z=377.1 [M+H]⁺.

### Step 6. Synthesis of tert-butyl (4-(2,2-dimethyl-4-oxo-3,8,11-trioxa-5-azatetradecan-14-amido)-3-((4,5-dimethylthiazol-2-yl)carbamoyl)phenyl)(methyl)carbamate

A solution of tert-butyl (4-amino-3-((4,5-dimethylthiazol-2-yl)carbamoyl)phenyl)(methyl)carbamate (500 mg, 1.33 mmol), 2,2-dimethyl-4-oxo-3,8,11-trioxa-5-azatetradecan-14-oic acid (368 mg, 1.33 mmol), HATU (1.01 g, 2.66 mmol) and DIEA (343 mg, 2.66 mmol) in DMF (5 mL) was stirred at rt for 2 h. The residue was purified by reverse-phase HPLC (0.1% FA in water and ACN) to provide the title compound (200 mg, 24% yield) as a brown solid. MS (ESI) m/z=636.4 [M+H]⁺.

### Step 7. Synthesis of 2-(3-(2-(2-aminoethoxy)ethoxy)propanamido)-N-(4,5-dimethylthiazol-2-yl)-5-(methylamino)benzamide

A solution of tert-butyl (4-(2,2-dimethyl-4-oxo-3,8,11-trioxa-5-azatetradecan-14-amido)-3-((4,5-dimethylthiazol-2-yl)carbamoyl)phenyl)(methyl)carbamate (200 mg, 0.315 mmol) in TFA (2 mL) and DCM (2 mL) was stirred at rt for 2 h. Upon completion, the mixture was concentrated in vacuo to provide the title compound (TFA salt, 150 mg, 87% yield) as yellow oil. ¹HNMR (400 MHz, DMSO-d₆) δ 7.73 (brs, 4H), 7.055 (brs, 1H), 6.80-6.76 (m, 1H), 3.67 (t, J=5.6 Hz, 2H), 3.58-3.52 (m, 6H), 2.95-2.91 (m, 2H), 2.74 (s, 3H), 2.53-2.50 (m, 2H), 2.25 (s, 3H), 2.19 (s, 3H). MS (ESI) m/z=436.1 [M+H]⁺.

### Example 270. 2-(3-(2-(2-Aminoethoxy)ethoxy)propanamido)-5-(dimethylamino)-N-(4,5-dimethylthiazol-2-yl)benzamide (BL1-146)

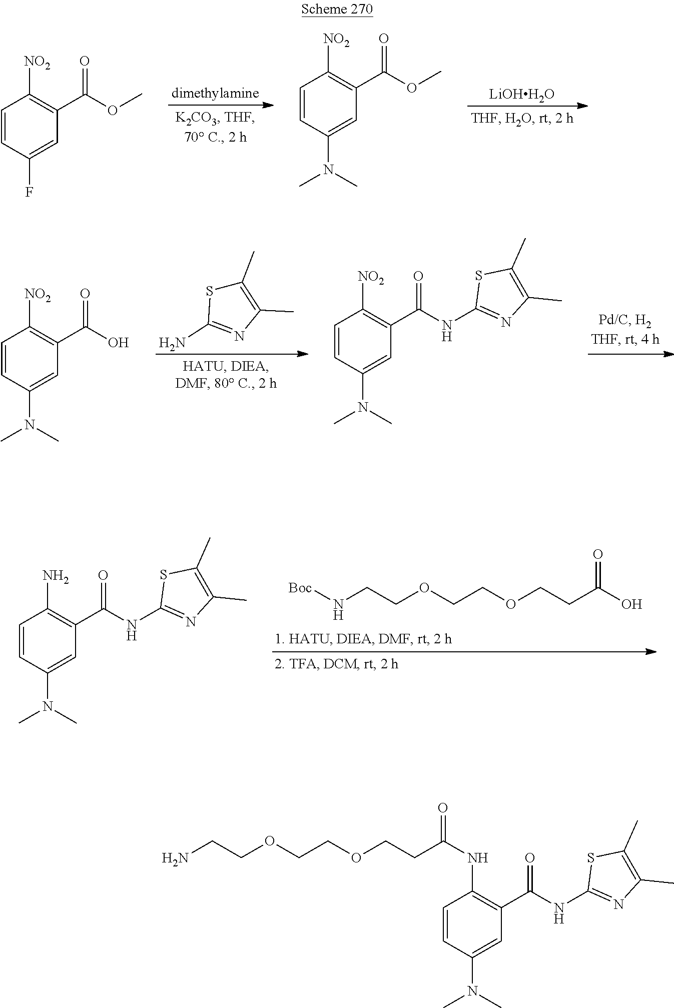

BL1-146 was synthesized following the standard procedure for preparing BL1-143 (TFA salt, 90 mg, 4% yield over 6 steps) as a yellow oil. ¹HNMR (400 MHz, DMSO-d₆) δ 10.36 (brs, 1H), 7.90 (brs, 1H), 7.74 (brs, 3H), 7.21 (brs, 1H), 6.96-6.94 (m, 1H), 3.68 (t, J=6.4 Hz, 2H), 3.56-3.54 (m, 6H), 2.92-2.91 (m, 8H), 2.54 (t, J=5.6 Hz, 2H), 2.26 (s, 3H), 2.20 (s, 3H). MS (ESI) m/z=450.2 [M+H]⁺.

### Example 271. 2-(3-(2-(2-Aminoethoxy)ethoxy)propanamido)-N-(4,5-dimethylthiazol-2-yl)-5-fluorobenzamide (BL1-147)

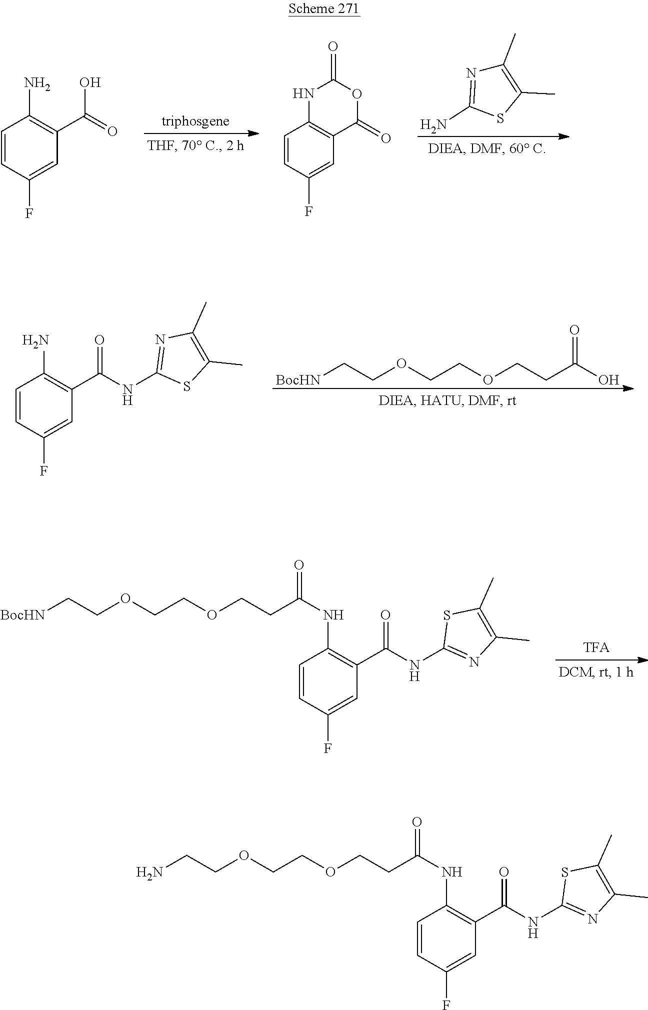

BL1-147 was synthesized following the standard procedure for preparing BL1-144 (372 mg, 13% yield over 4 steps) as a yellow solid. ¹HNMR (400 MHz, DMSO-d₆) δ 11.3 (brs, 1H), 8.30 (s, 1H), 7.79-7.69 (m, 4H), 7.39-7.35 (m, 1H), 3.87-3.85 (m, 2H), 3.73-3.70 (m, 6H), 2.94-2.91 (m, 2H), 2.65-2.62 (m, 2H), 2.25 (s, 3H), 2.20 (s, 3H). MS (ESI) m/z=425.1 [M+H]⁺.

### Example 272. 4-((2-((4,5-Dimethylthiazol-2-yl)carbamoyl)phenyl)amino)-4-oxobutanoic acid (BL1-148)

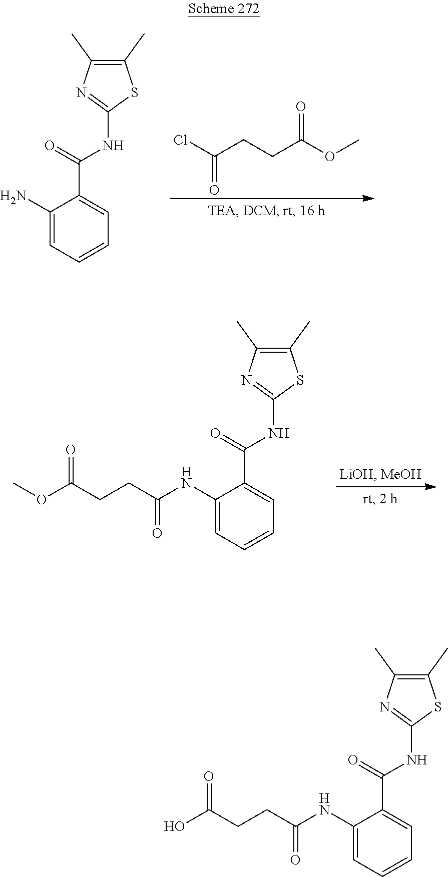

BL1-148 was synthesized following the standard procedure for preparing BL1-123 (202 mg, 36% yield over 2 steps) as a white solid. MS (ESI) m/z=348.0 [M+H]⁺.

### Example 273. 3-(2-(2-Aminoethoxy)ethoxy)-N-(2-(((4,5-dimethylthiazol-2-yl)amino)methyl)phenyl)propanamide (BL1-149)

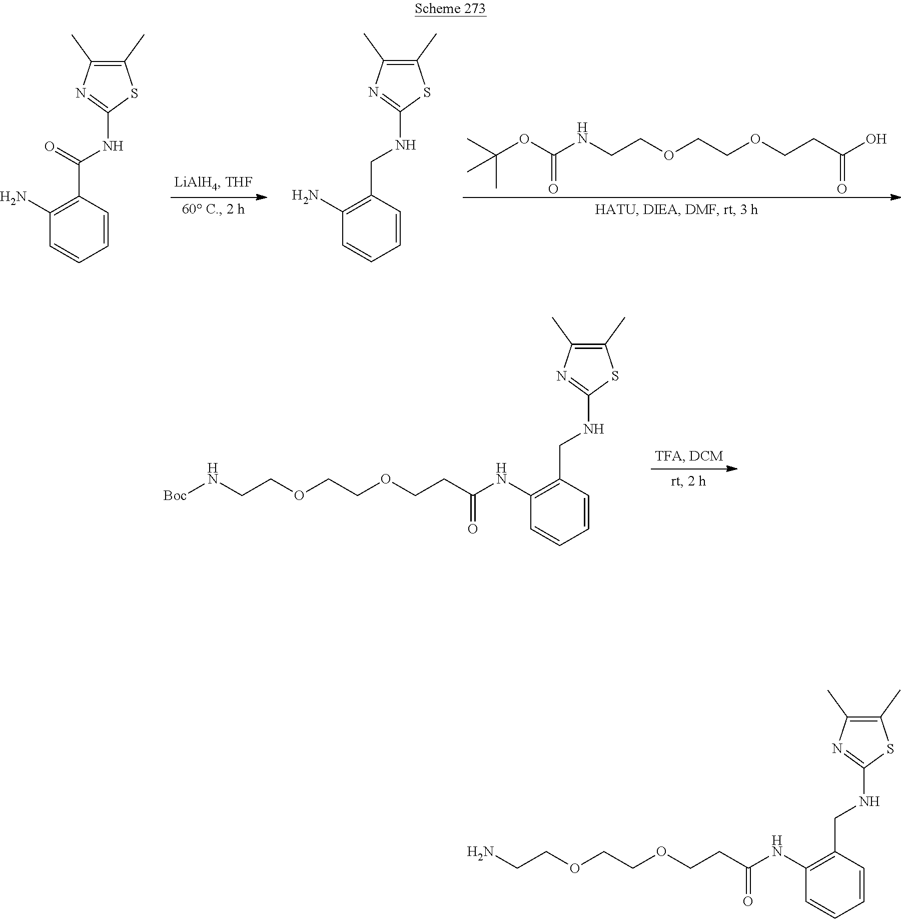 [SMILES: Cc1nc(NC(=O)c2ccccc2N)sc1C ; Cc1nc(NCc2ccccc2N)sc1C ; CC(C)(C)OC(=O)NCCOCCOCCC(=O)O ; Cc1nc(NCc2ccccc2NC(=O)CCOCCOCCNC(=O)OC(C)(C)C)sc1C ; Cc1nc(NCc2ccccc2NC(=O)CCOCCOCCN)sc1C]

### Step 1. Synthesis of N-(2-aminobenzyl)-4,5-dimethylthiazol-2-amine

To a solution of 2-amino-N-(4,5-dimethylthiazol-2-yl)benzamide (600 mg, 2.43 mmol) in THF (10 mL) at 0° C. was added LiAlH₄ (184 mg, 4.86 mmol). The mixture was heated at 60° C. for 2 h. After cooled to rt, the mixture was quenched with aq. NaOH solution (10%, 15 mL) and filtered. The filtrate was extracted with EtOAc (30 mL×3). The organic phases were combined and washed with brine, dried over Na₂SO₄, filtered and concentrated in vacuo to provide the title compound (640 mg, crude) as a yellow solid, which was used for the next step directly. MS (ESI) m/z=234.2 [M+H]⁺.

### Step 2. Synthesis of tert-butyl (2-(2-(3-((2-(((4,5-dimethylthiazol-2-yl)amino)methyl)phenyl)amino)-3-oxopropoxy)ethoxy)ethyl)carbamate

A solution of N-(2-aminobenzyl)-4,5-dimethylthiazol-2-amine (320 mg, crude), 2,2-dimethyl-4-oxo-3,8,11-trioxa-5-azatetradecan-14-oic acid (673 mg, 2.43 mmol), HATU (1.05 g, 2.74 mmol) and DIEA (626 mg, 4.80 mmol) in DMF (5 ml) was stirred at rt for 3 h. The mixture was purified by reverse-phase HPLC (0.1% TFA in water and ACN) to provide the title compound (160 mg, 27% yield over 2 steps) as a brown oil. MS (ESI) m/z=493.3 [M+H]⁺.

### Step 3. Synthesis of 3-(2-(2-aminoethoxy)ethoxy)-N-(2-(((4,5-dimethylthiazol-2-yl)amino)methyl)phenyl)propenamide

A solution of tert-butyl (2-(2-(3-((2-(((4,5-dimethylthiazol-2-yl)amino)methyl)phenyl)amino)-3-oxopropoxy)ethoxy)ethyl)carbamate (160 mg, 0.407 mmol) in TFA (3 mL) and DCM (3 mL) was stirred at rt for 2 h. Upon completion, the mixture was concentrated in vacuo. The residue was purified by reverse-phase HPLC (0.1% TFA in water and ACN) to provide the title compound (83.1 mg, 52% yield) as a brown oil. ¹HNMR (400 MHz, DMSO-d₆) δ 9.91 (brs, 1H), 7.78 (brs, 3H), 7.42 (d, J=8.0 Hz, 1H), 7.35-7.20 (m, 3H), 4.41 (d, J=1.6 Hz, 2H), 3.74 (t, J=6.4 Hz, 2H), 3.60-3.53 (m, 6H), 2.98-2.94 (m, 2H), 2.62 (t, J=6.0 Hz, 2H), 2.13 (s, 3H), 2.09 (s, 3H). MS (ESI) m/z=393.2 [M+H]⁺.

### Example 274. 2-(3-(2-(2-Aminoethoxy)ethoxy)propanamido)-4-(dimethylamino)-N-(4,5-dimethylthiazol-2-yl)benzamide (BL1-150)

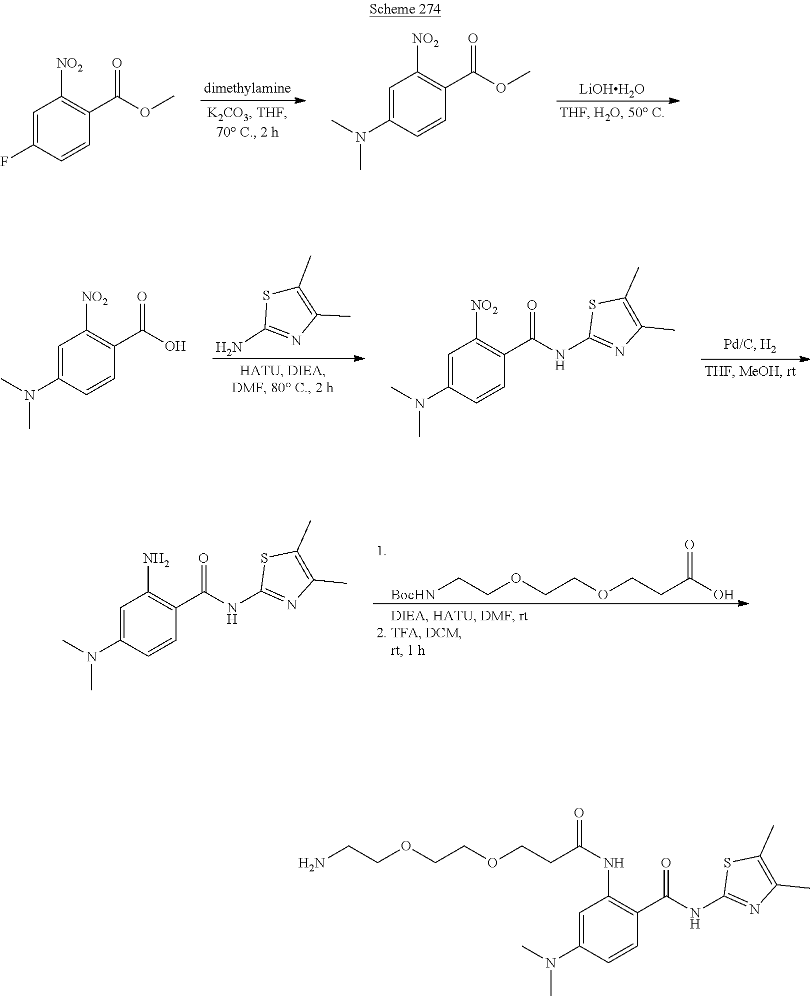

BL1-150 was synthesized following the standard procedure for preparing BL1-143 (TFA salt, 372 mg, 6% yield over 6 steps) as a yellow solid. ¹HNMR (400 MHz, DMSO-d₆) δ 11.72 (brs, 1H), 7.99-7.98 (m, 2H), 7.69 (brs, 3H), 6.44-6.42 (m, 1H), 3.74 (t, J=6.0 Hz, 2H), 3.59-3.54 (m, 6H), 2.99 (s, 6H), 2.92-2.91 (m, 2H), 2.63-2.60 (m, 2H), 2.24 (s, 3H), 2.19 (s, 3H). MS (ESI) m/z=450.0 [M+H]⁺.

### Example 275. 6-((2-((4,5-Dimethylthiazol-2-yl)carbamoyl)phenyl)amino)-6-oxohexanoic acid (BL1-151)

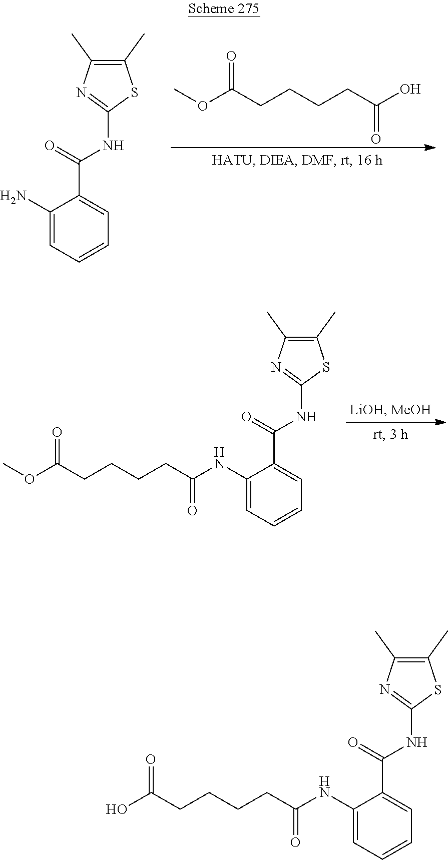

BL1-151 was synthesized following the standard procedure for preparing BL1-171 (302 mg, 50% yield over 2 steps) as a white solid. MS (ESI) m/z=375.9 [M+H]⁺.

### Example 276. 7-((2-((4,5-Dimethylthiazol-2-yl)carbamoyl)phenyl)amino)-7-oxoheptanoic acid (BL1-152)

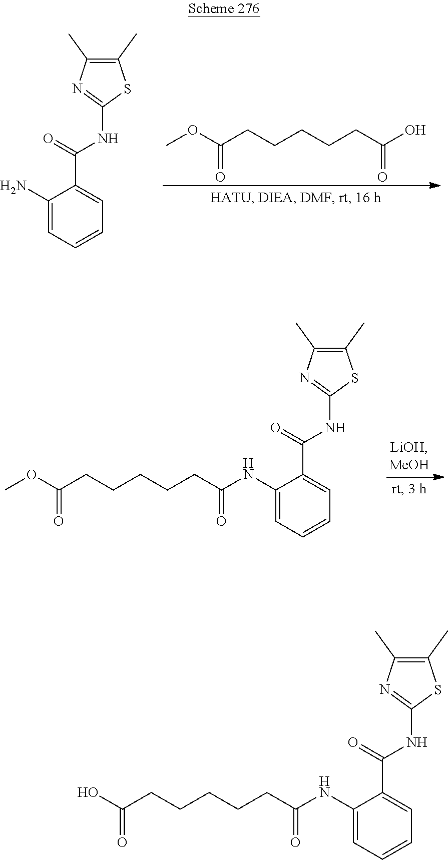

BL1-152 was synthesized following the standard procedure for preparing BL1-171 (178 mg, 28% yield over 2 steps) as a white solid. MS (ESI) m/z=389.9 [M+H]⁺.

### Example 277. 3-((2-((4,5-Dimethylthiazol-2-yl)carbamoyl)phenyl)amino)-3-oxopropanoic acid (BL1-153)

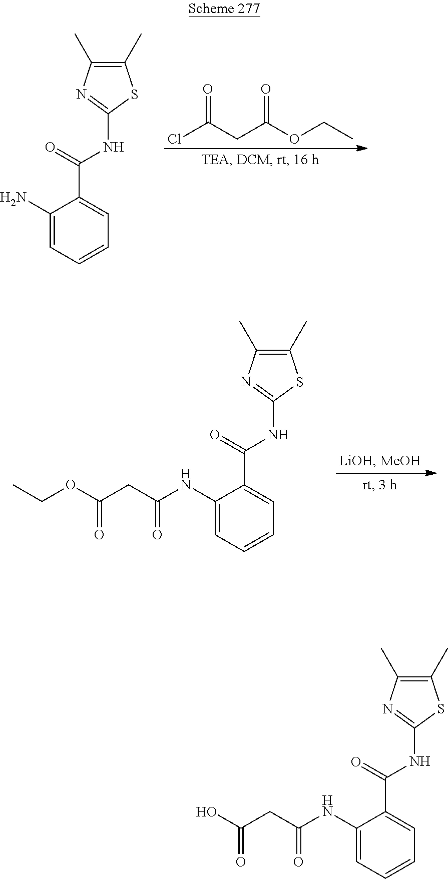

BL1-153 was synthesized following the standard procedure for preparing BL1-123 (172 mg, 32% yield over 2 steps) as a white solid. MS (ESI) m/z=334.0 [M+H]⁺.

### Example 278. 5-((2-((4,5-Dimethylthiazol-2-yl)carbamoyl)phenyl)amino)-5-oxopentanoic acid (BL1-154)

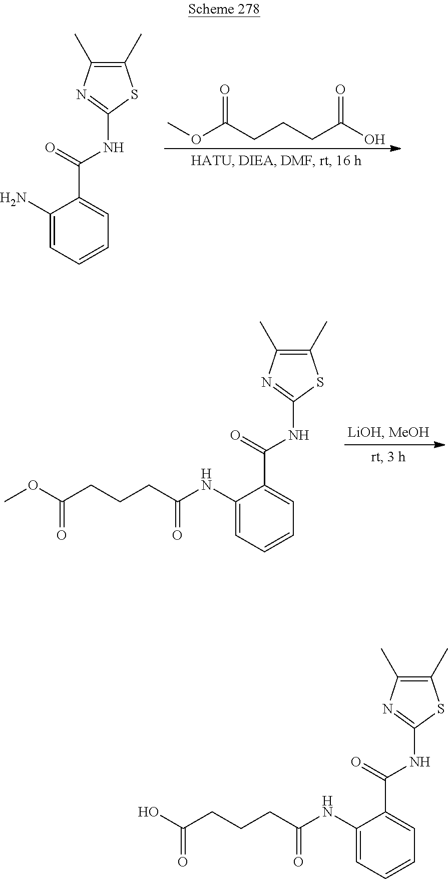

BL1-154 was synthesized following the standard procedure for preparing BL1-171 (178 mg, 40% yield over 2 steps) as a white solid. MS (ESI) m/z=389.9 [M+H]⁺.

### Example 279. 9-((2-((4,5-Dimethylthiazol-2-yl)carbamoyl)phenyl)amino)-9-oxononanoic acid (BL1-155)

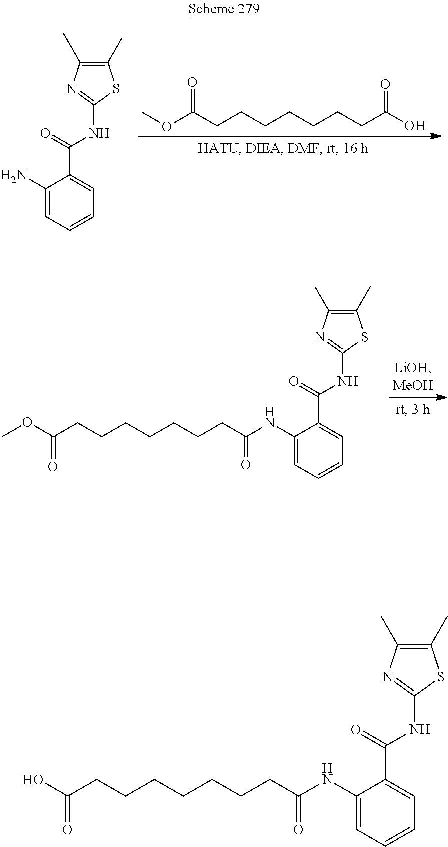

BL1-155 was synthesized following the standard procedure for preparing BL1-171 (318 mg, 47% yield over 2 steps) as a white solid. MS (ESI) m/z=417.9 [M+H]⁺.

### Example 280. 8-((2-((4,5-Dimethylthiazol-2-yl)carbamoyl)phenyl)amino)-8-oxooctanoic acid (BL1-156)

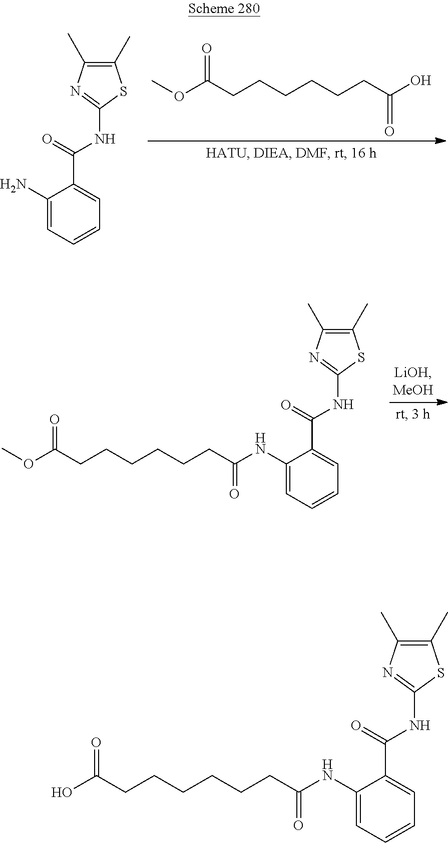

BL1-156 was synthesized following the standard procedure for preparing BL1-171 (402 mg, yield: 62% over 2 steps) as a white solid. MS (ESI) m/z=404.1 [M+H]⁺.

### Example 281. 10-((2-((4,5-Dimethylthiazol-2-yl)carbamoyl)phenyl)amino)-10-oxodecanoic acid (BL1-157)

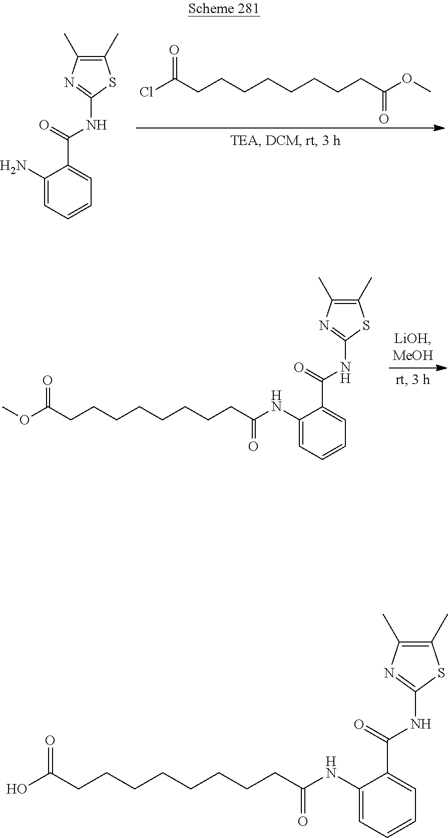

BL1-157 was synthesized following the standard procedure for preparing BL1-123 (203 mg, 29% yield over 2 steps) as a white solid. MS (ESI) m/z=431.9 [M+H]⁺.

### Example 282. 19-((2-((4,5-Dimethylthiazol-2-yl)carbamoyl)phenyl)amino)-19-oxo-4,7,10,13,16-pentaoxanonadecanoic acid (BL1-158)

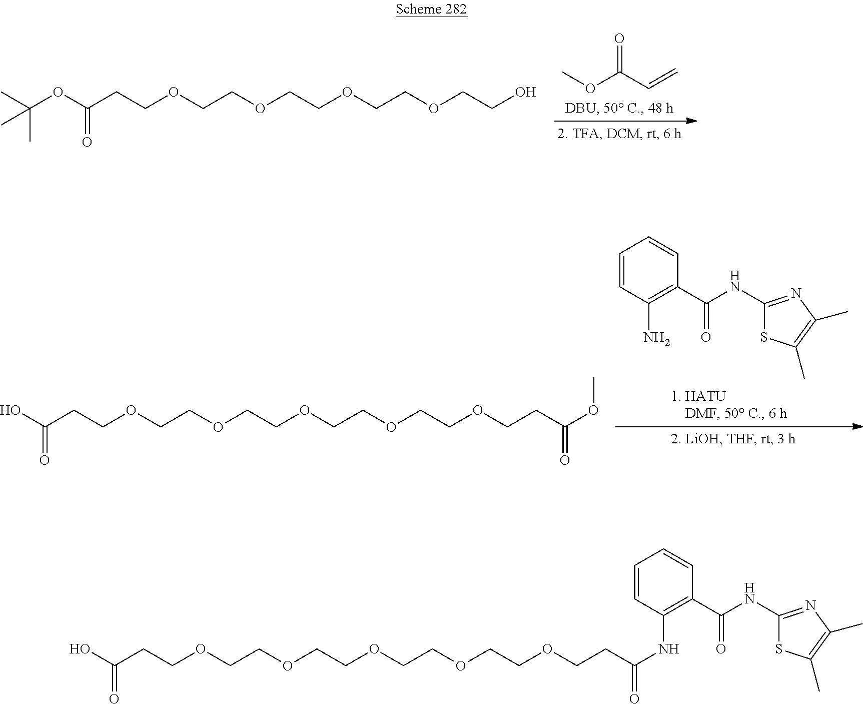

BL1-158 was synthesized following the standard procedure for preparing BL1-171 (270 mg, 16% yield over 4 steps) as a white solid. MS (ESI) m/z=568.2 [M+H]⁺.

### Example 283. 2-(3-(2-(2-Aminoethoxy)ethoxy)propanamido)-N-(4,5-dimethylthiazol-2-yl)-4-(methylamino)benzamide (BL1-159)

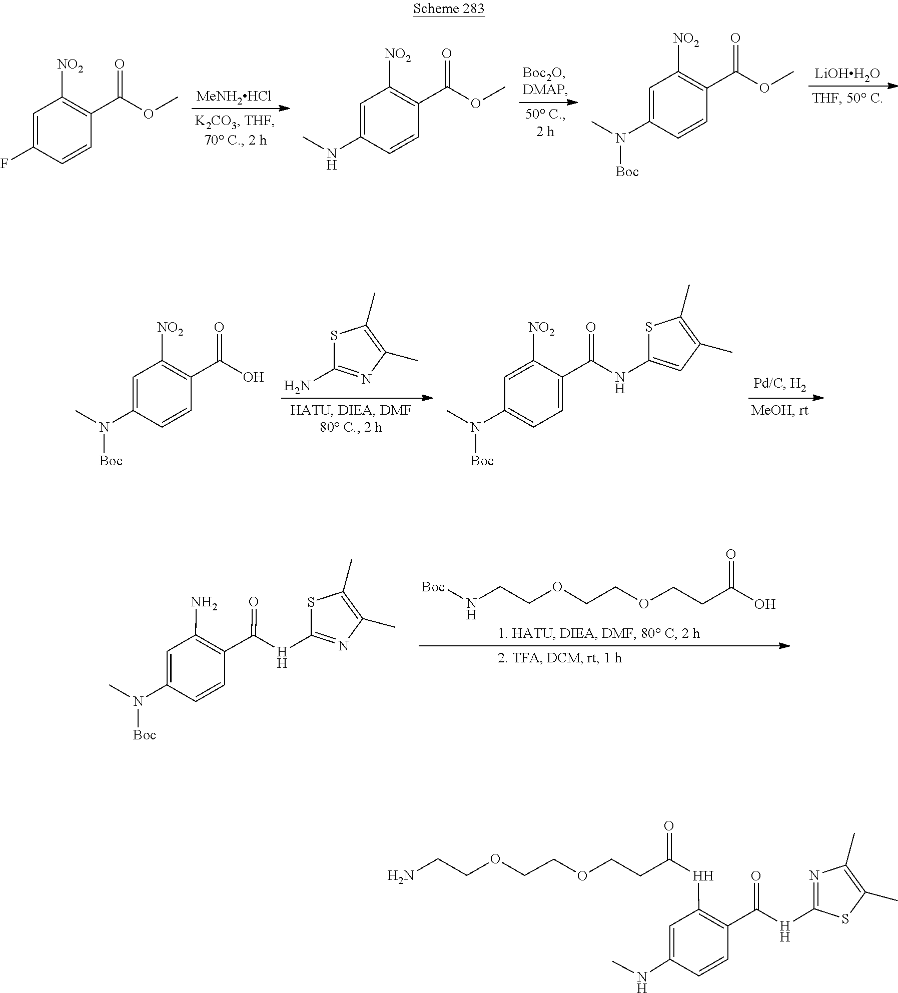

BL1-159 was synthesized following the standard procedure for preparing BL1-145 (TFA salt, 99.1 mg, 16% yield over 7 steps) as a white solid. ¹HNMR (400 MHz, DMSO-d₆) δ 11.75 (brs, 1H), 7.93-7.91 (m, 1H), 7.82-7.81 (m, 1H), 7.74-7.70 (m, 3H), 6.29-6.26 (m, 1H), 3.75 (t, J=6.0 Hz, 2H), 3.62-3.55 (m, 6H), 2.95-2.91 (m, 2H), 2.73 (s, 3H), 2.61 (t, J=6.0 Hz, 2H), 2.25 (s, 3H), 2.20 (s, 3H). MS (ESI) m/z=436.2 [M+H]⁺.

### Example 284. 4-((3-((4,5-Dimethylthiazol-2-yl)carbamoyl)-4-methylphenyl)(4-methoxybenzyl)amino)butanoic acid (BL1-160)

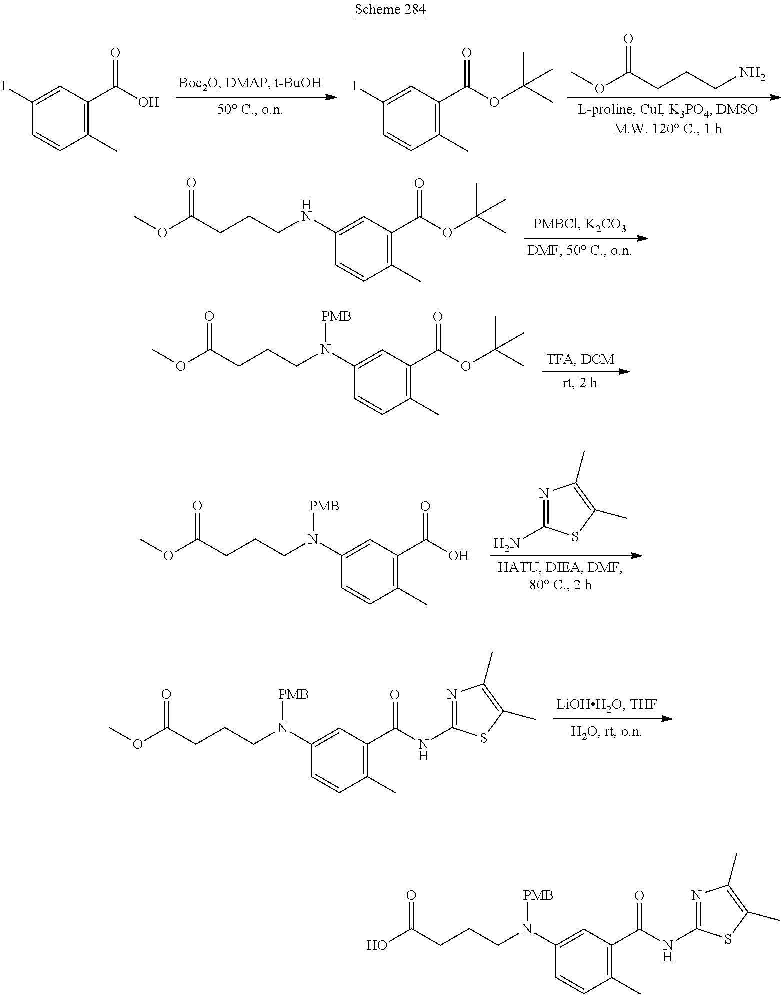

### Step 1. Synthesis of tert-butyl 5-iodo-2-methylbenzoate

A solution of 5-iodo-2-methylbenzoic acid (5.00 g, 19.1 mmol), Boc₂O (8.33 g, 38.2 mmol) and DMAP (233 mg, 1.91 mmol) in t-BuOH (60 mL) was stirred at 50° C. overnight. Upon completion, the mixture was concentrated under reduced pressure. The residue was purified by silica gel chromatography (petroleum ether/EtOAc=10:1) to provide the title compound (5.00 g, 82% yield) as a colorless oil.

### Step 2. Synthesis of tert-butyl 5-((4-methoxy-4-oxobutyl)amino)-2-methylbenzoate

A solution of tert-butyl 5-iodo-2-methylbenzoate (1.50 g, 4.72 mmol), methyl 4-aminobutanoate (1.11 g, 9.44 mmol), CuI (902 mg, 4.72 mmol), L-proline (543 mg, 4.72 mmol) and K₃PO₄ (3.00 g, 14.2 mmol) in DMSO (6 mL) was heated at 120° C. for 1 h in the microwave reactor under inert atmosphere. After cooled to rt, the mixture was purified by reverse-phase HPLC (0.1% TFA in water and ACN) to provide the title compound (650 mg, 45% yield) as a white solid. MS (ESI) m/z=308.2 [M+H]⁺.

### Step 3. Synthesis of tert-butyl 5-((4-methoxy-4-oxobutyl)(4-methoxybenzyl)amino)-2-methylbenzoate

A solution of tert-butyl 5-((4-methoxy-4-oxobutyl)amino)-2-methylbenzoate (550 mg, 1.79 mmol), 1-(chloromethyl)-4-methoxybenzene (838 mg, 5.37 mmol) and K₂CO₃ (741 mg, 5.37 mmol) in DMF (5 mL) was stirred at 50° C. overnight. After cooled to rt, the mixture was diluted with water (50 mL) and extracted with EtOAc (50 mL×3). The combined organic phase was washed with brine (50 mL×2), dried over Na₂SO₄, filtered and concentrated in vacuo. The residue was purified by silica gel chromatography (petroleum ether/EtOAc=8:1) to afford the title compound (300 mg, 39% yield) as a white solid. MS (ESI) m/z=428.2 [M+H]⁺.

### Step 4. Synthesis of 5-((4-methoxy-4-oxobutyl)(4-methoxybenzyl)amino)-2-methylbenzoic acid

A solution of tert-butyl 5-((4-methoxy-4-oxobutyl)(4-methoxybenzyl)amino)-2-methylbenzoate (300 mg, 0.703 mmol) in TFA (2 mL) and DCM (2 mL) was stirred at rt for 2 h. Upon completion, the mixture was concentrated in vacuo to provide the title compound (220 mg, 85% yield) as a brown solid. MS (ESI) m/z=372.1 [M+H]⁺.

### Step 5. Synthesis of methyl 4-((3-((4,5-dimethylthiazol-2-yl)carbamoyl)-4-methylphenyl)(4-methoxybenzyl)amino)butanoate

To a solution of 5-((4-methoxy-4-oxobutyl)(4-methoxybenzyl)amino)-2-methylbenzoic acid (100 mg, 0.270 mmol), 4,5-dimethylthiazol-2-amine (69 mg, 0.540 mmol) and HATU (205 mg, 0.540 mmol) in DMF (4 mL) was added DIEA (140 mg, 1.08 mmol) at 80° C. The mixture was stirred at 80° C. for 2 h. Water (50 mL) was added. The mixture was extracted with EtOAc (50 mL×3). The combined organic phase was washed with brine (50 mL×2), dried over Na₂SO₄, filtered and concentrated in vacuo. The residue was purified by silica gel chromatography (petroleum ether/EtOAc=2:1) to provide the title compound (100 mg, 77% yield) as a yellow oil. MS (ESI) m/z=482.3 [M+H]⁺.

### Step 6. Synthesis of 4-((3-((4,5-dimethylthiazol-2-yl)carbamoyl)-4-methylphenyl)(4-methoxybenzyl)amino)butanoic acid

A solution of methyl 4-((3-((4,5-dimethylthiazol-2-yl)carbamoyl)-4-methylphenyl)(4-methoxybenzyl)amino)butanoate (100 mg, 0.208 mmol), LiOH·H₂O (44 mg, 1.04 mmol) in THF (5 mL) and H₂O (5 mL) was stirred at rt overnight. Upon completion, the mixture was diluted with water (50 mL) and acidified by aq. HCl solution (1 M) to pH=2. The mixture was extracted with EtOAc (50 mL×3). The combined organic phase was washed with brine, dried over Na₂SO₄, filtered and concentrated in vacuo to provide the title compound (70.0 mg, 72% yield) as a white solid. ¹HNMR (400 MHz, DMSO-d₆) δ 12.12 (s, 2H), 7.12 (d, J=8.4 Hz, 2H), 7.02 (d, J=8.4 Hz, 1H), 6.88-6.85 (m, 3H), 6.73 (dd, J=8.8 Hz, 2.8 Hz, 1H), 4.51 (s, 2H), 3.72 (s, 3H), 3.41-3.37 (m, 2H), 2.29-2.27 (m, 5H), 2.22 (s, 3H), 2.18 (s, 3H), 1.84-1.78 (m, 2H). MS (ESI) m/z=468.2 [M+H]⁺.

### Example 285. N¹-(4-(((4,5-Dimethylthiazol-2-yl)amino)methyl)-3-methylphenyl)-3,6,9,12,15-pentaoxaheptadecane-1,17-diamine (BL1-161)

### Step 1. Synthesis of (4-iodo-2-methylphenyl)methanol

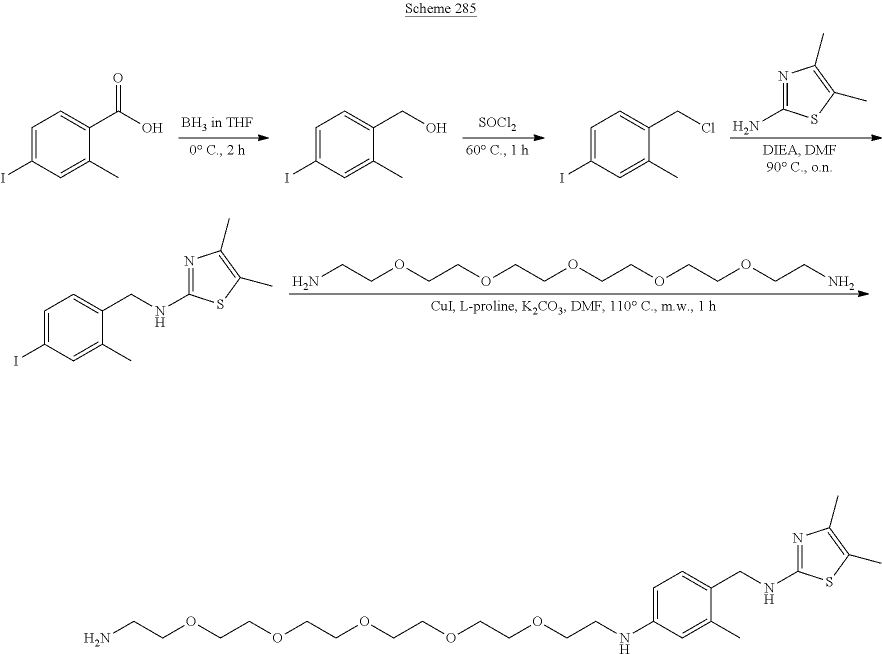

A solution of 4-iodo-2-methylbenzoic acid (2.00 g, 7.63 mmol) in BH₃-THF (1 M in THF, 20 mL) was stirred at 0° C. for 2 h. The mixture was quenched with MeOH (10 mL) and concentrated in vacuo. The residue was purified by silica gel chromatography (petroleum ether/EtOAc=2:1) to provide the tittle compound (1.60 g, 85% yield) as a colorless oil. MS (ESI) m/z=249.1 [M+H]⁺.

### Step 2. Synthesis of 1-(chloromethyl)-4-iodo-2-methylbenzene

A solution of (4-iodo-2-methylphenyl)methanol (1.60 g, 6.45 mmol) in thionyl chloride (10 mL) was stirred at 60° C. for 1 h. The mixture was concentrated in vacuo. The residue was purified by silica gel chromatography (petroleum ether/EtOAc=5:1) to provide the title compound (1.50 g, 87% yield) as a colorless oil.

### Step 3. Synthesis of N-(4-iodo-2-methylbenzyl)-4,5-dimethylthiazol-2-amine

A solution of 1-(chloromethyl)-4-iodo-2-methylbenzene (1.50 g, 5.64 mmol), 4,5-dimethylthiazol-2-amine (866 mg, 6.77 mmol) and DIEA (1.45 g, 11.3 mmol) in DMF (15 mL) was stirred at 90° C. overnight. After cooled to rt, the residue was diluted with water (30 mL) and extracted with EtOAc (30 mL×3). The combined organic phase was washed with brine, dried over Na₂SO₄, filtered and concentrated. The residue was purified by silica gel chromatography (petroleum ether/EtOAc=0:1) to provide the title compound (500 mg, 25% yield) as a yellow solid. MS (ESI) m/z=359.1 [M+H]⁺.

### Step 4. Synthesis of N¹-(4-(((4,5-dimethylthiazol-2-yl)amino)methyl)-3-methylphenyl)-3,6,9,12,15-pentaoxaheptadecane-1,17-diamine

A solution of N-(4-iodo-2-methylbenzyl)-4,5-dimethylthiazol-2-amine (200 mg, 0.558 mmol), 3,6,9,12,15-pentaoxaheptadecane-1,17-diamine (467 mg, 1.67 mmol), L-proline (64 mg, 0.558 mmol), CuI (106 mg, 0.806 mmol) and K₂CO₃ (230 mg, 1.67 mmol) in DMSO (3 mL) was stirred at 110° C. for 1 h in the microwave reactor under N₂. After cooled to rt, the mixture was purified by reverse-phase HPLC (0.1% TFA in water and ACN) to provide the title compound (TFA salt, 60.0 mg, 17% yield) as a yellow oil. ¹HNMR (400 MHz, DMSO-d₆) δ 7.74 (brs, 4H), 7.02 (d, J=8.4 Hz, 1H), 6.50-6.41 (m, 2H), 4.33 (d, J=3.2 Hz, 2H), 3.60-3.52 (m, 20H), 3.19-3.16 (m, 2H), 2.99-2.96 (m, 2H), 2.18 (s, 3H), 2.14 (s, 3H), 2.11 (s, 3H). MS (ESI) m/z=511.3 [M+H]⁺.

### Example 286. 8-((3-((4,5-Dimethylthiazol-2-yl)carbamoyl)-2-methylphenyl)amino)octanoic acid (BL1-162)

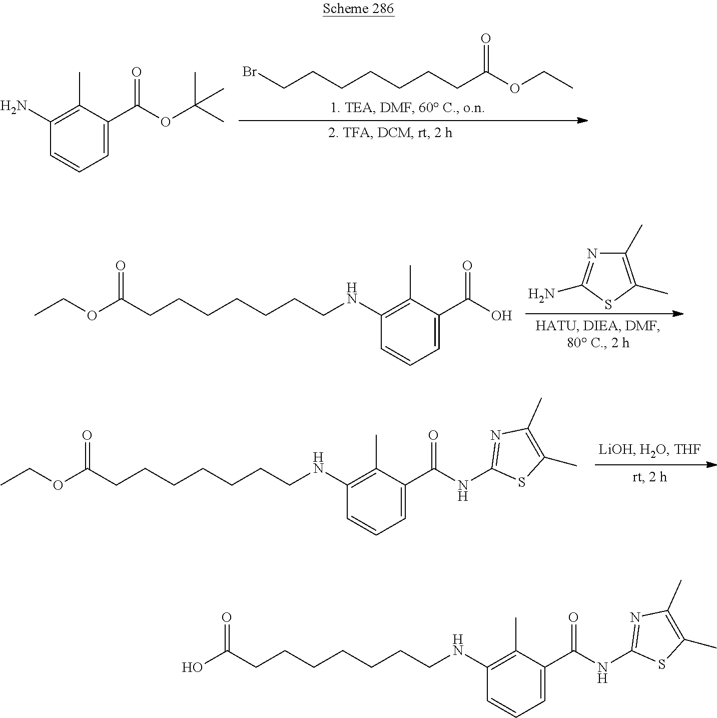

BL1-162 was synthesized following the standard procedure for preparing BL1-120 (35.4 mg, 6% over 4 steps) as a white solid. ¹HNMR (400 MHz, DMSO-d₆) δ 12.02 (brs, 1H), 11.9 (brs, 1H), 7.09 (t, J=7.8 Hz, 1H), 6.64 (t, J=7.2 Hz, 2H), 4.99 (t, J=5.6 Hz, 1H), 3.11-3.08 (m, 2H), 2.27 (s, 3H), 2.21 (t, J=7.2 Hz, 2H), 2.17 (s, 3H), 2.05 (s, 3H), 1.60-1.49 (m, 4H), 1.31-1.26 (m, 6H). MS (ESI) m/z=404.4 [M+H]⁺.

### Example 287. 2-(3-(2-(2-Aminoethoxy)ethoxy)propanamido)-4-(butylamino)-N-(4,5-dimethylthiazol-2-yl)benzamide (BL1-163)

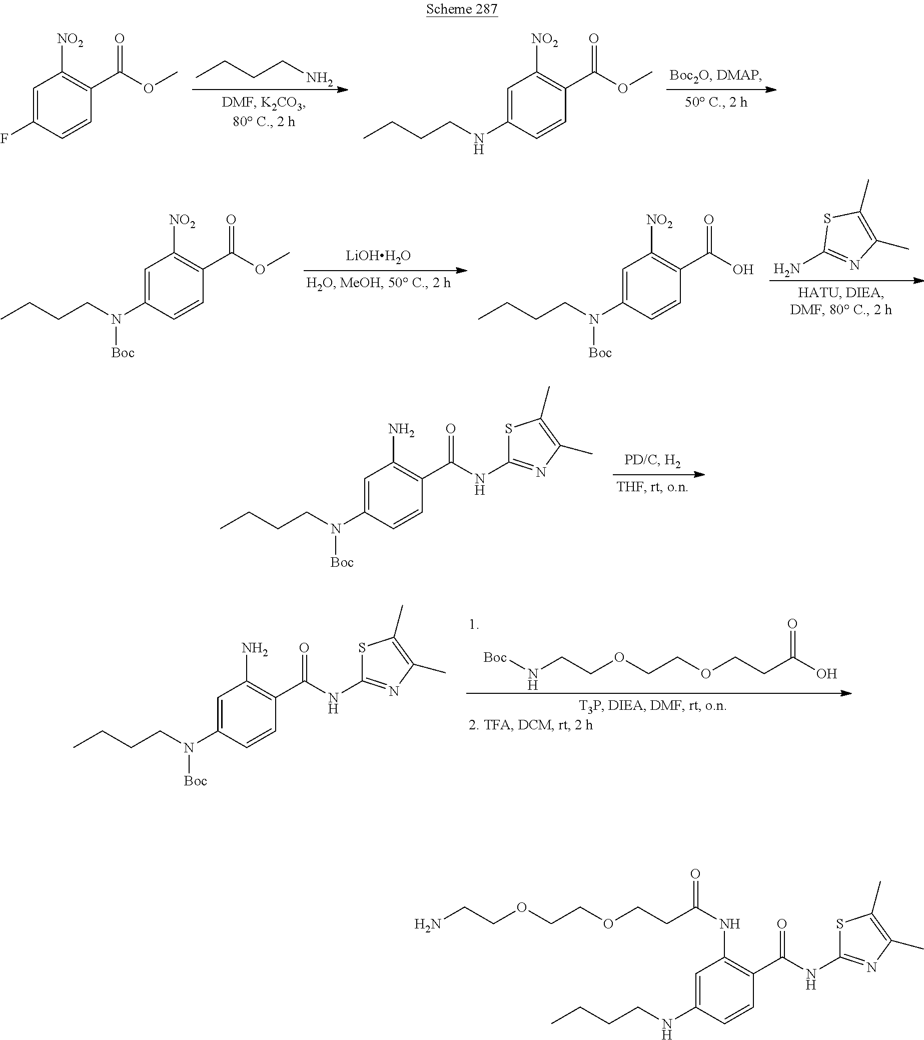

BL1-163 was synthesized following the standard procedure for preparing BL1-145 (TFA salt, 49.0 mg, 5% yield over 7 steps) as yellow oil. ¹HNMR (400 MHz, DMSO-d₆) δ 11.73 (brs, 1H), 7.89 (d, J=9.2 Hz, 1H), 7.84 (d, J=2.0 Hz, 1H), 7.73 (brs, 3H), 6.30 (dd, J=8.8 Hz, 2.4 Hz, 1H), 3.75 (t, J=5.6 Hz, 2H), 3.62-3.56 (m, 6H), 3.06 (t, J=7.2 Hz, 2H), 2.95-2.91 (m, 2H), 2.61 (t, J=6.0 Hz, 2H), 2.25 (s, 3H), 2.20 (s, 3H), 1.57-1.51 (m, 2H), 1.43-1.34 (m, 2H), 0.93 (t, J=7.2 Hz, 3H). MS (ESI) m/z=478.3 [M+H]⁺.

### Example 288. 2-(7-Aminoheptanamido)-N-(4,5-dimethylthiazol-2-yl)benzamide (BL1-164)

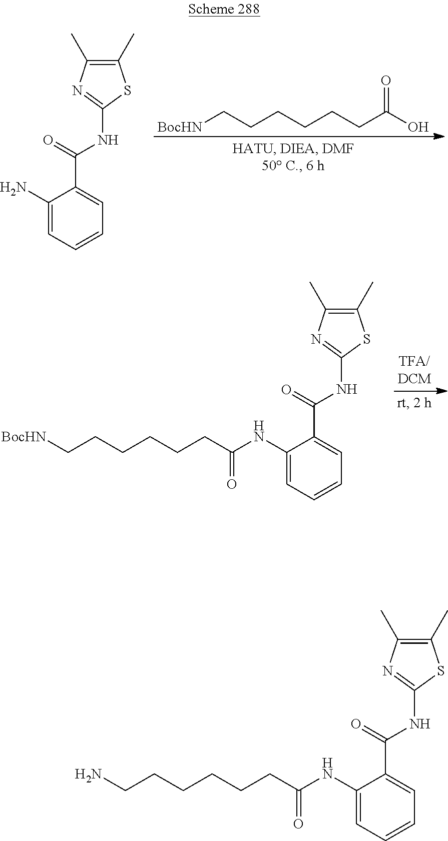 [SMILES: Cc1nc(NC(=O)c2ccccc2N)sc1C ; CC(C)(C)OC(=O)NCCCCCCC(=O)O ; Cc1nc(NC(=O)c2ccccc2NC(=O)CCCCCCNC(=O)OC(C)(C)C)sc1C ; Cc1nc(NC(=O)c2ccccc2NC(=O)CCCCCCN)sc1C]

BL1-164 was synthesized following the standard procedure for preparing BL1-144 (617 mg, 75% yield over 2 steps) as a white solid. MS (ESI) m/z=375.0 [M+H]⁺.

### Example 289. 1-Amino-N-(2-((4,5-dimethylthiazol-2-yl)carbamoyl)phenyl)-3,6,9,12-tetraoxapentadecan-15-amide (BL1-165)

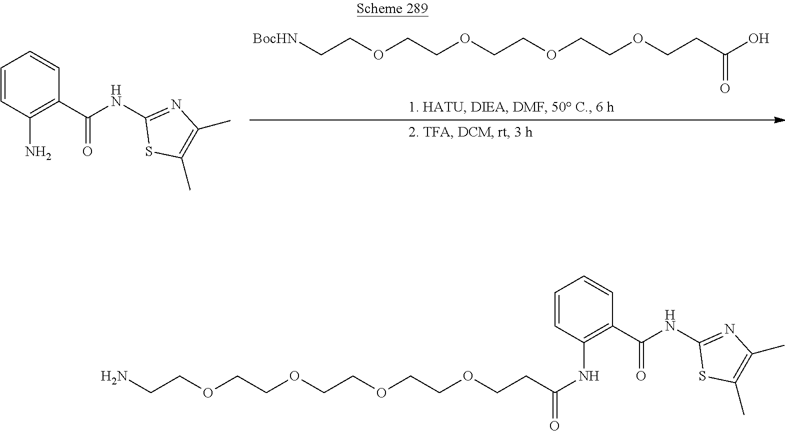 [SMILES: Cc1nc(NC(=O)c2ccccc2N)sc1C ; CC(C)(C)OC(=O)NCCOCCOCCOCCOCCC(=O)O ; Cc1nc(NC(=O)c2ccccc2NC(=O)CCOCCOCCOCCOCCN)sc1C]

BL1-165 was synthesized following the standard procedure for preparing BL1-144 (480 mg, yield: 49% over 2 steps) as a yellow solid. MS (ESI) m/z=494.9 [M+H]⁺.

### Example 290. 2-(4-Aminobutanamido)-N-(4,5-dimethylthiazol-2-yl)benzamide (BL1-166)

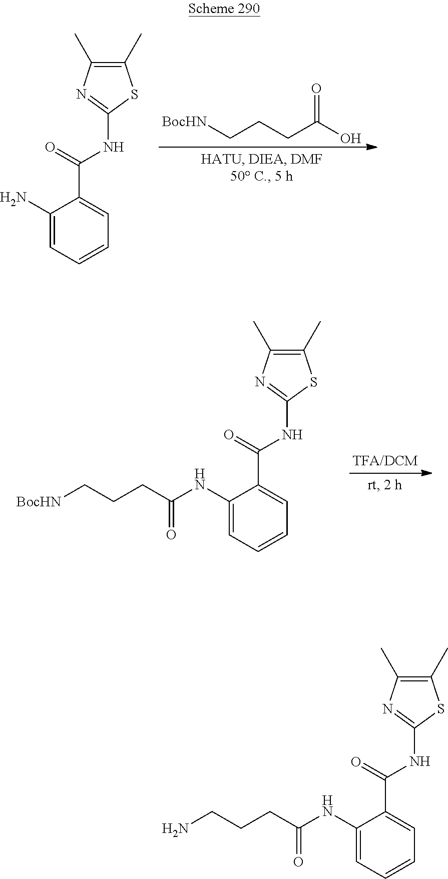 [SMILES: Cc1nc(NC(=O)c2ccccc2N)sc1C ; CC(C)(C)OC(=O)NCCCC(=O)O ; Cc1nc(NC(=O)c2ccccc2NC(=O)CCCNC(=O)OC(C)(C)C)sc1C ; Cc1nc(NC(=O)c2ccccc2NC(=O)CCCN)sc1C]

BL1-166 was synthesized following the standard procedure for preparing BL1-144 (53.2 mg, 35% yield over 2 steps) as a white solid. MS (ESI) m/z=333.0 [M+H]⁺.

### Example 291. 2-(4-Aminobutanamido)-N-(4,5-dimethylthiazol-2-yl)benzamide (BL1-167)

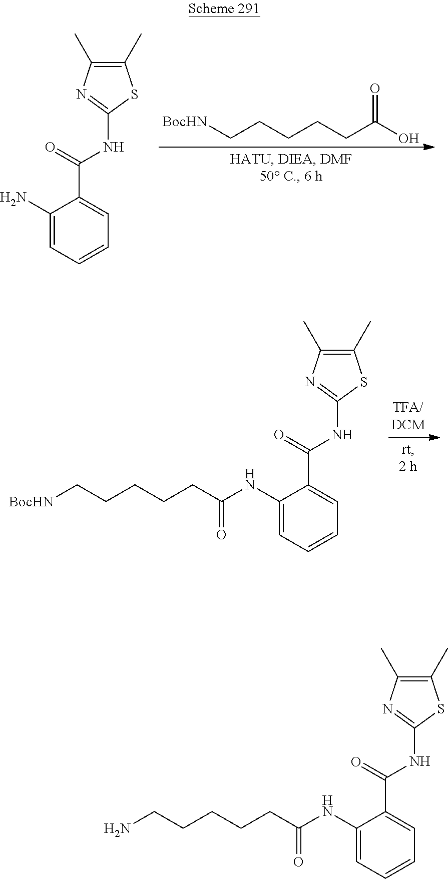 [SMILES: Cc1nc(NC(=O)c2ccccc2N)sc1C ; CC(C)(C)OC(=O)NCCCCCC(=O)O ; Cc1nc(NC(=O)c2ccccc2NC(=O)CCCCCNC(=O)OC(C)(C)C)sc1C ; Cc1nc(NC(=O)c2ccccc2NC(=O)CCCCCN)sc1C]

BL1-167 was synthesized following the standard procedure for preparing BL1-144 (710 mg, 46% yield over 2 steps) as a white solid. MS (ESI) m/z=361.0 [M+H]⁺.

### Example 292. 1-Amino-N-(2-((4,5-dimethylthiazol-2-yl)carbamoyl)phenyl)-3,6,9,12,15,18-hexaoxahenicosan-21-amide (BL1-168)

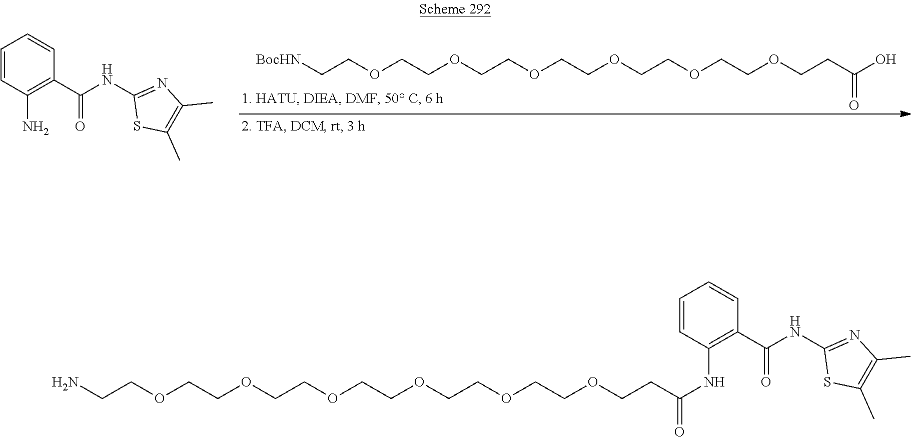 [SMILES: Cc1nc(NC(=O)c2ccccc2N)sc1C ; CC(C)(C)OC(=O)NCCOCCOCCOCCOCCOCCOCCC(=O)O ; Cc1nc(NC(=O)c2ccccc2NC(=O)CCOCCOCCOCCOCCOCCOCCN)sc1C]

BL1-168 was synthesized following the standard procedure for preparing BL1-144 (470 mg, 41% yield over 2 steps) as a yellow solid. MS (ESI) m/z=583.2 [M+H]⁺.

### Example 293. 2-(5-Aminopentanamido)-N-(4,5-dimethylthiazol-2-yl)benzamide (BL1-169)

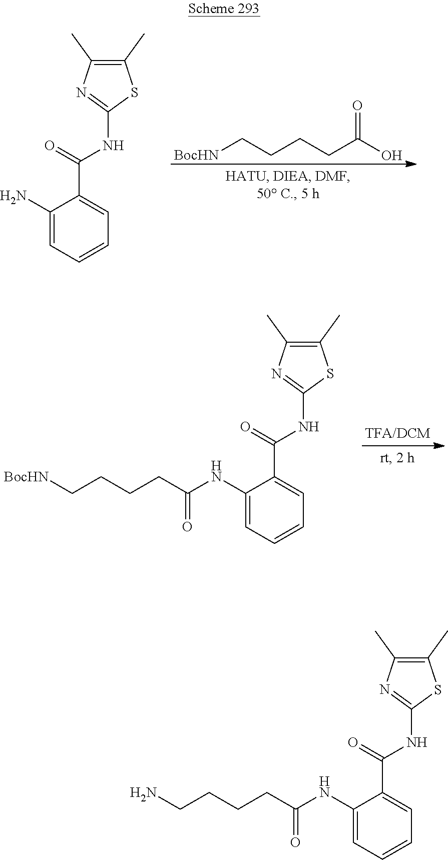 [SMILES: Cc1nc(NC(=O)c2ccccc2N)sc1C ; CC(C)(C)OC(=O)NCCCCC(=O)O ; Cc1nc(NC(=O)c2ccccc2NC(=O)CCCCNC(=O)OC(C)(C)C)sc1C ; Cc1nc(NC(=O)c2ccccc2NC(=O)CCCCN)sc1C]

BL1-169 was synthesized following the standard procedure for preparing BL1-144 (384 mg, 89% yield over 2 steps) as a white solid. MS (ESI) m/z=347.1 [M+H]⁺.

### Example 294. 1-Amino-N-(2-((4,5-dimethylthiazol-2-yl)carbamoyl)phenyl)-3,6,9,12,15-pentaoxaoctadecan-18-amide (BL1-170)

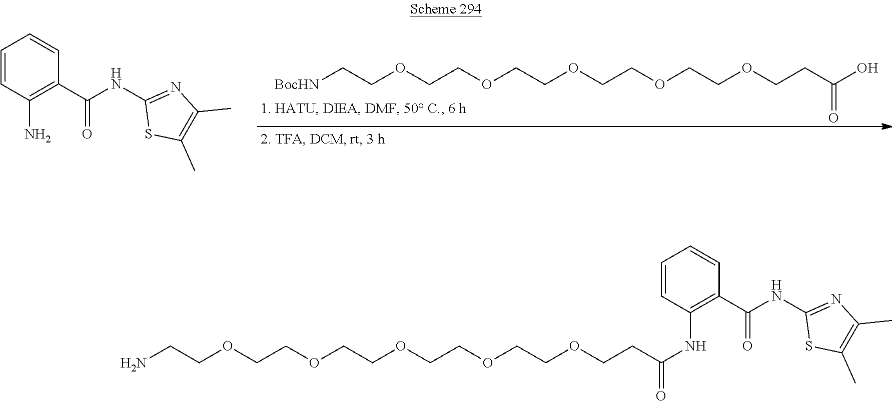 [SMILES: Cc1nc(NC(=O)c2ccccc2N)sc1C ; CC(C)(C)OC(=O)NCCOCCOCCOCCOCCOCCC(=O)O ; Cc1nc(NC(=O)c2ccccc2NC(=O)CCOCCOCCOCCOCCOCCN)sc1C]

BL1-170 was synthesized following the standard procedure for preparing BL1-144 (770 mg, 72% yield over 2 steps) as a yellow solid. MS (ESI) m/z=538.9 [M+H]⁺.

### Example 295. 16-((2-((4,5-Dimethylthiazol-2-yl)carbamoyl)phenyl)amino)-16-oxo-4,7,10,13-tetraoxahexadecanoic acid (BL1-171)

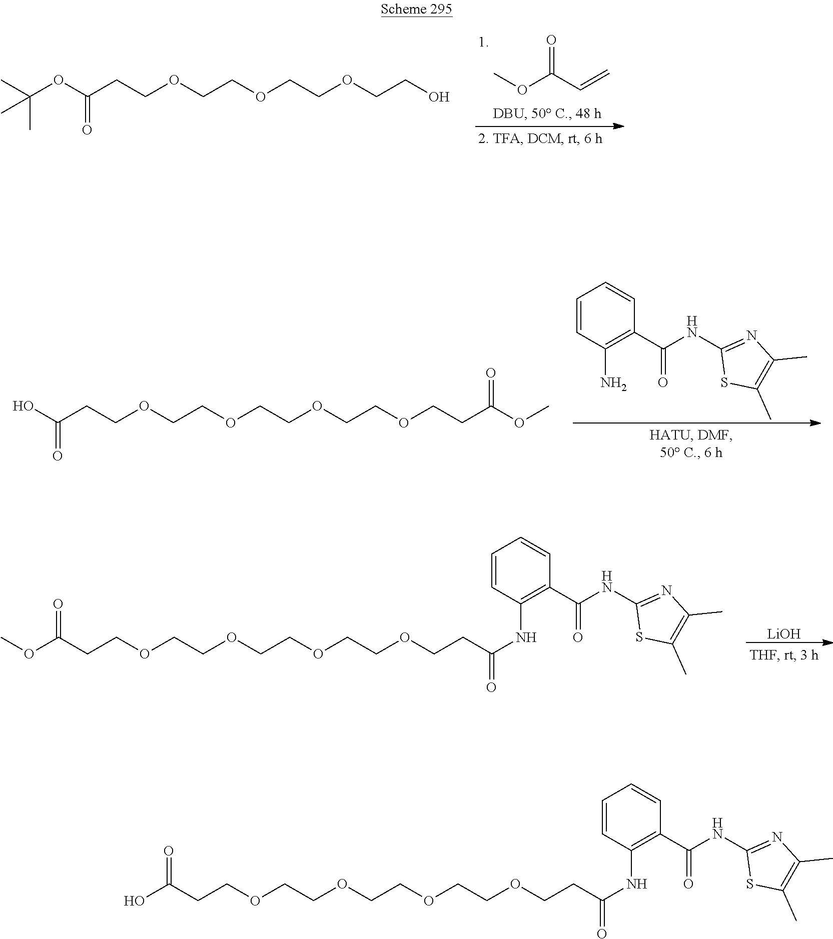

BL1-171 was synthesized following the standard procedure for preparing BL1-177 (410 mg, 13% yield over 4 steps) as a white solid. MS (ESI) m/z=523.8 [M+H]⁺.

### Example 296. 16-((2-((4,5-Dimethylthiazol-2-yl)carbamoyl)phenyl)amino)-16-oxo-4,7,10,13-tetraoxahexadecanoic acid (BL1-172)

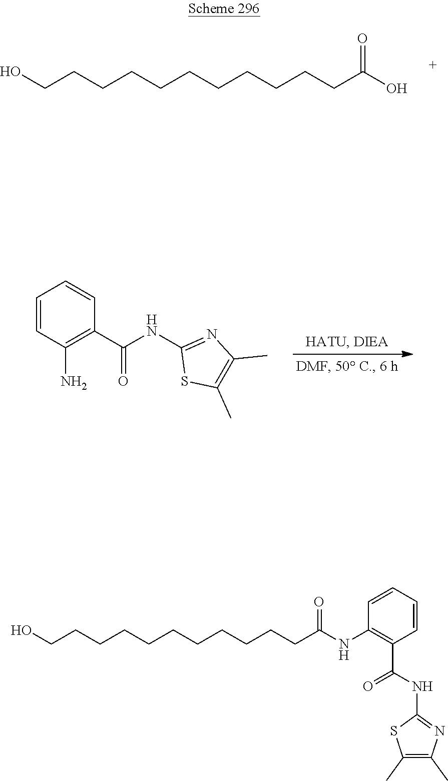 [SMILES: O=C(O)CCCCCCCCCCCO ; Cc1nc(NC(=O)c2ccccc2N)sc1C ; Cc1nc(NC(=O)c2ccccc2NC(=O)CCCCCCCCCCCO)sc1C]

BL1-172 was synthesized following the standard procedure for preparing BL1-144 (250 mg, 56% yield) as a white solid. MS (ESI) m/z=445.9 [M+H]⁺.

### Example 297. 2-(3-(2-(2-Aminoethoxy)ethoxy)propanamido)-N-(4,5-dimethylthiazol-2-yl)cyclohexane-1-carboxamide (BL1-174)

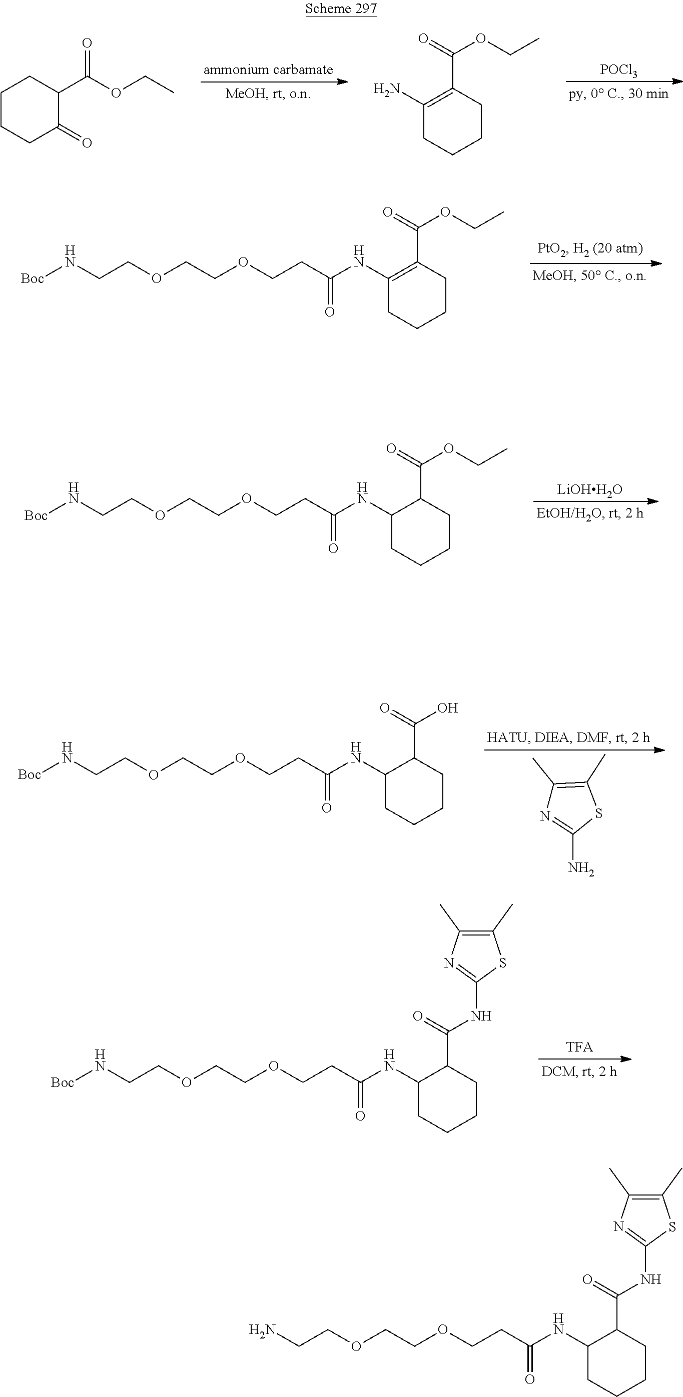

### Step 1. Synthesis of ethyl 2-aminocyclohex-1-ene-1-carboxylate

A solution of ethyl 2-oxocyclohexane-1-carboxylate (5.00 g, 29.4 mmol) and ammonium carbamate (11.5 g, 147 mmol) in MeOH (50 mL) was stirred at rt overnight. The mixture was diluted with water (200 mL) and extracted with EtOAc (100 mL×2). The combined organic phase was washed with brine (200 mL×2), dried over Na₂SO₄, filtered and concentrated in vacuo. The residue was purified by silica gel chromatography (petroleum ether/EtOAc=5:1) to provide the title compound (2.00 g, crude) as a colorless oil, which was used for next step directly. MS (ESI) m/z=170.1 [M+H]⁺.

### Step 2. Synthesis of ethyl 2-(2,2-dimethyl-4-oxo-3,8,11-trioxa-5-azatetradecan-14-amido)cyclohex-1-ene-1-carboxylate

To a solution of ethyl 2-aminocyclohex-1-ene-1-carboxylate (2.00 g, crude) in pyridine (20 mL) at 0° C., was added POCl₃ (1.81 g, 11.8 mmol). The mixture was stirred at 0° C. for 30 min. Upon completion, ice water (100 mL) was added slowly. The mixture was extracted with EtOAc (50 mL×2). The combined organic phase was washed with aqueous HCl solution (1 M, 100 mL) and brine, dried over Na₂SO₄, filtered and concentrated in vacuo. The residue was purified by silica gel chromatography (petroleum ether/EtOAc=5:1) to provide the title compound (500 mg, 4% yield over two steps) as a colorless oil. MS (ESI) m/z=451.2 [M+Na]⁺.

### Step 3. Synthesis of ethyl 2-(2,2-dimethyl-4-oxo-3,8,11-trioxa-5-azatetradecan-14-amido)cyclohexane-1-carboxylate

A solution of ethyl 2-(2,2-dimethyl-4-oxo-3,8,11-trioxa-5-azatetradecan-14-amido)cyclohex-1-ene-1-carboxylate (500 mg, 1.17 mmol) and PtO₂ (796 mg, 3.50 mmol) in MeOH (5 mL) was stirred at 50° C. overnight under H₂ (20 atm). After cooled to rt, the mixture was filtered and concentrated in vacuo. The residue was purified by prep-HPLC (0.1% FA in water and ACN) to provide the title compound (70 mg, 14% yield) as a colorless oil. MS (ESI) m/z=431.6 [M+H]⁺.

### Step 4. Synthesis of 2-(2,2-dimethyl-4-oxo-3,8,11-trioxa-5-azatetradecan-14-amido)cyclohexane-1-carboxylic acid

A solution of ethyl 2-(2,2-dimethyl-4-oxo-3,8,11-trioxa-5-azatetradecan-14-amido)cyclohexane-1-carboxylate (70 mg, 0.163 mmol) and LiOH·H₂O (34 mg, 0.813 mmol) in EtOH (2 mL) and H₂O (1 mL) was stirred at rt for 2 h. Then the mixture was diluted with water (10 mL) and acidified to pH=4 with aq. HCl solution (1 M). The mixture was extracted with EtOAc (20 mL×2). The combined organic phase was washed with brine (20 mL×2), dried over Na₂SO₄, filtered and concentrated in vacuo to provide the title compound (35 mg, 53% yield) as a colorless oil. MS (ESI) m/z=403.2 [M+H]⁺.

### Step 5. Synthesis of tert-butyl (2-(2-(3-((2-((4,5-dimethylthiazol-2-yl)carbamoyl)cyclohexyl)amino)-3-oxopropoxy)ethoxy)ethyl)carbamate

A solution of 2-(2,2-dimethyl-4-oxo-3,8,11-trioxa-5-azatetradecan-14-amido)cyclohexane-1-carboxylic acid (35 mg, 0.0871 mmol), 4,5-dimethylthiazol-2-amine (17 mg, 0.131 mmol), HATU (50 mg, 0.131 mmol) and DIEA (22 mg, 0.174 mmol) in DMF (1 mL) was stirred at rt for 2 h. The mixture was purified by prep-HPLC (0.1% FA in water and ACN) to provide the title compound (40 mg, 90% yield) as a colorless oil. MS (ESI) m/z=513.2 [M+H]⁺.

### Step 6. Synthesis of 2-(3-(2-(2-aminoethoxy)ethoxy)propanamido)-N-(4,5-dimethylthiazol-2-yl)cyclohexane-1-carboxamide

A solution of tert-butyl (2-(2-(3-((2-((4,5-dimethylthiazol-2-yl)carbamoyl)cyclohexyl)amino)-3-oxopropoxy)ethoxy)ethyl)carbamate (40 mg, 0.0781 mmol) in TFA (1 mL) and DCM (1 mL) was stirred at rt for 2 h. The mixture was concentrated in vacuo to provide the title compound (TFA salt, 25 mg, 61% yield) as a brown oil. ¹HNMR (400 MHz, DMSO-d₆) δ 11.69 (brs, 1H), 7.73 (brs, 2H), 7.68 (d, J=8.8 Hz, 1H), 4.30-4.27 (m, 1H), 3.55-3.44 (m, 8H), 2.98-2.91 (m, 2H), 2.77-2.74 (m, 1H), 2.35-2.26 (m, 2H), 2.21 (s, 3H), 2.13 (s, 3H), 1.88-1.77 (m, 2H), 1.57-1.23 (m, 6H). MS (ESI) m/z=413.2 [M+H]⁺.

### Example 298. 2-(2-Aminoacetamido)-N-(4,5-dimethylthiazol-2-yl)benzamide (CLI-yy-C001, BL1-175)

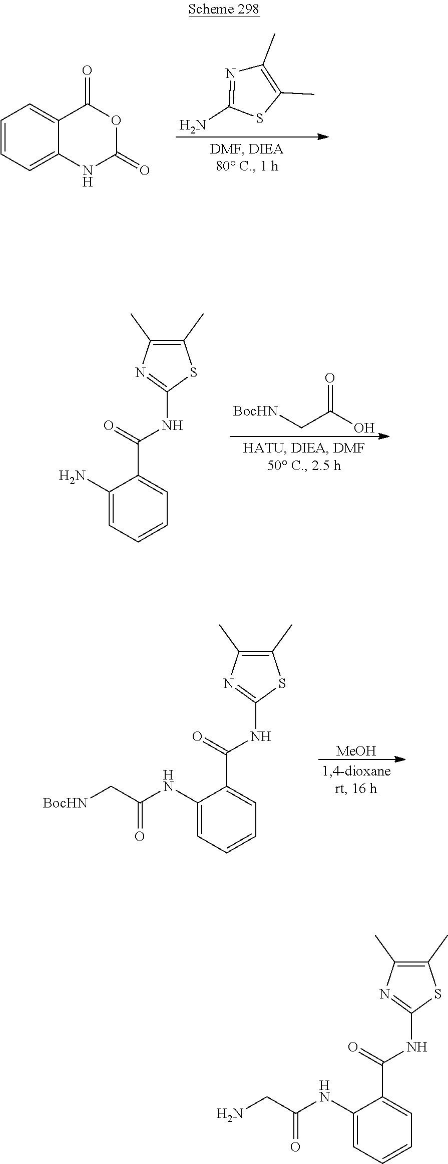

BL1-175 was synthesized following the standard procedure for preparing BL1-144 (140 mg, 19% yield over 3 steps) as a white solid. MS (ESI) m/z=304.9 [M+H]⁺.

### Example 299. 3-(3-((2-((4,5-Dimethylthiazol-2-yl)carbamoyl)phenyl)amino)-3-oxopropoxy)propanoic acid (BL1-176)

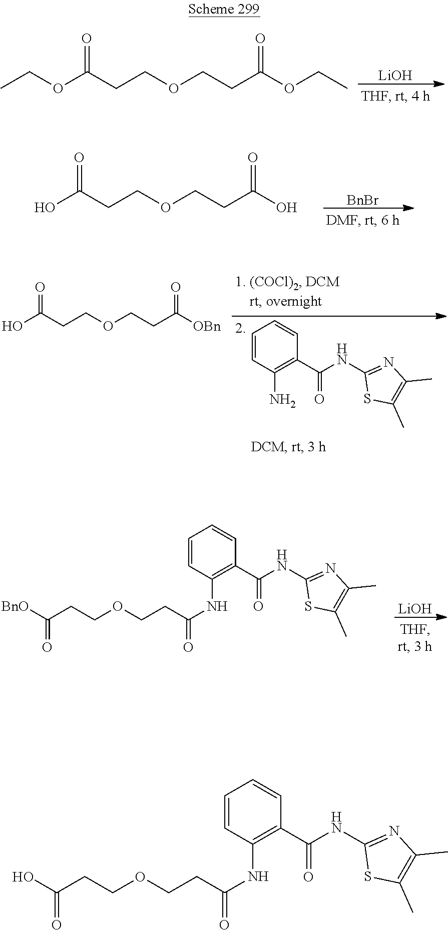

### Step 1. Synthesis of 3,3′-oxydipropionic acid

To a solution of diethyl 3,3′-oxydipropionate (5.0 g, 22 mmol) in THF (40 mL) and H₂O (10 mL) was added LiOH (4.8 g, 114 mmol). The mixture was stirred at rt for 4 h. The reaction was monitored by TLC. Upon completion, the mixture was acidified to pH=1-2, and extracted with EtOAc. The organic layer was concentrated to afford the title compound (2.7 g, 70%) as a yellow solid.

### Step 2. Synthesis of 3-(3-(benzyloxy)-3-oxopropoxy)propanoic acid

To a solution of 3,3′-oxydipropionic acid (2.7 g, 16.7 mmol) in DMF (10 mL) were added DIEA (4.3 g, 33.3 mmol) and BnBr (2.85 g, 16.7 mmol). The mixture was stirred at rt for 16 h, and then acidified to pH=1-2. The mixture was extracted with EtOAc (20 ml×3). The combined organic layers were washed with brine (30 mL), dried over sodium sulfate, filtered and concentrated to afford the title compound (1.0 g, 23% yield) as a yellow oil.

### Step 3. Synthesis of 2-amino-N-(4,5-dimethylthiazol-2-yl)benzamide

To a solution of 3-(3-(benzyloxy)-3-oxopropoxy)propanoic acid (1.0 g, 4 mmol) in DCM (5 mL) were added oxalyl chloride (604 mg, 5 mmol) and DMF (1 drop). The mixture was stirred at rt for 16 h, then concentrated to provide the title compound (1.0 g, 93% yield) as a yellow oil.

### Step 4. Synthesis of diethyl benzyl 3-(3-((2-((4,5-dimethylthiazol-2-yl)carbamoyl)phenyl)amino)-3-oxopropoxy)propanoate

To a solution of 2-amino-N-(4,5-dimethylthiazol-2-yl)benzamide (400 mg, 1.6 mmol) in DCM (10 mL) were added DIEA (418 mg, 3.2 mmol) and benzyl 3-(3-chloro-3-oxopropoxy)propanoate (525 mg, 1.9 mmol). The mixture was stirred at rt for 3 h, then concentrated under reduced pressure. The residue was purified by reverse-phase chromatography (0.1% TFA in H₂O and ACN) to provide the title compound (500 mg, 64% yield) as a yellow oil. MS (ESI) m/z=482.2 [M+H]⁺.

### Step 5. Synthesis of 3-(3-((2-((4,5-dimethylthiazol-2-yl)carbamoyl)phenyl)amino)-3-oxopropoxy)propanoic acid

To a solution of diethyl benzyl 3-(3-((2-((4,5-dimethylthiazol-2-yl)carbamoyl)phenyl)amino)-3-oxopropoxy)propanoate (400 mg, 0.8 mmol) in THF (5 mL) and H₂O (2 mL) was added LiOH (175 mg, 4.2 mmol). The mixture was stirred at rt for 3 h. Upon completion, the mixture was acidified to pH=1-2, and extracted with EtOAc. The organic layer was concentrated to provide the title compound (290 mg, 89% yield) as a white solid. MS (ESI) m/z=391.9 [M+H]⁺.

### Example 300. 22-((2-((4,5-Dimethylthiazol-2-yl)carbamoyl)phenyl)amino)-22-oxo-4,7,10,13,16,19-hexaoxadocosanoic acid (BL1-177)

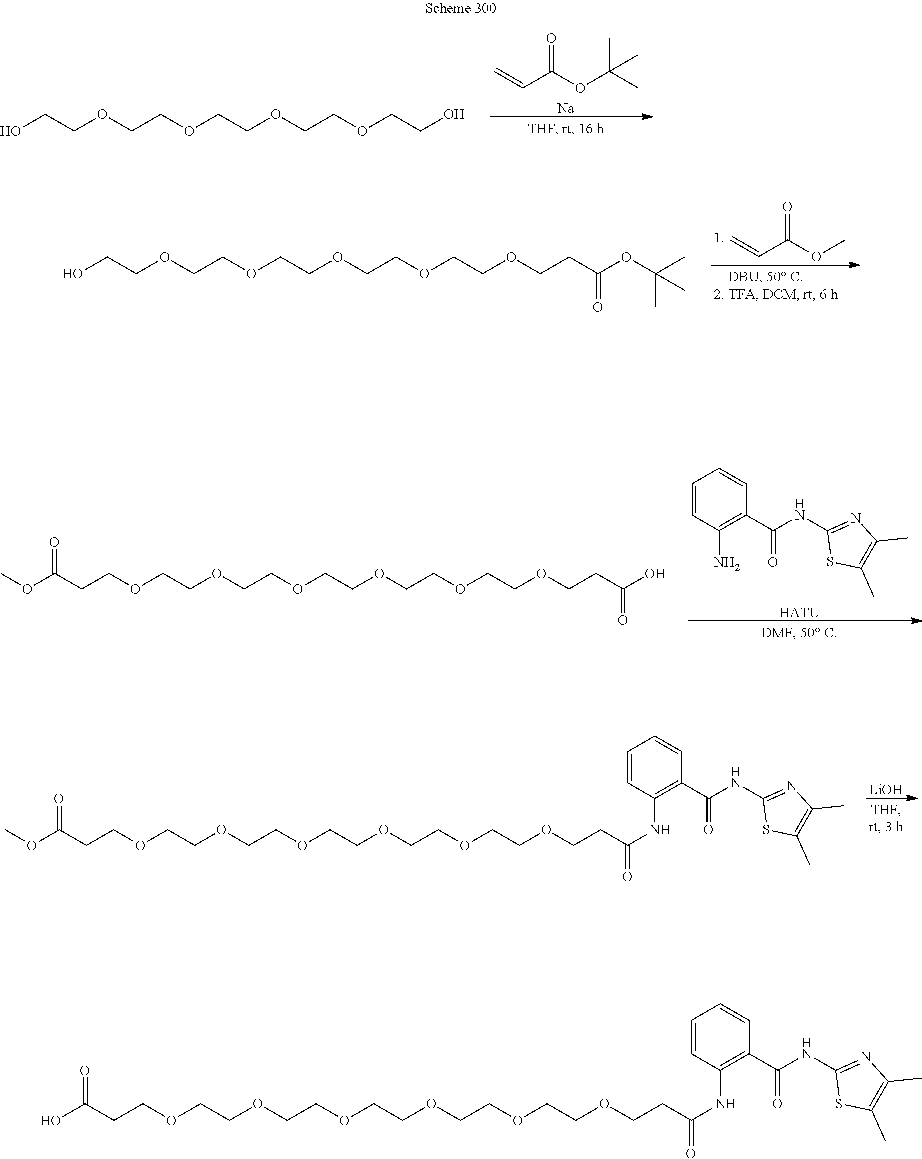

### Step 1. Synthesis of tert-butyl 1-hydroxy-3,6,9,12,15-pentaoxaoctadecan-18-oate

To a solution of pentaethylene glycol (16.7 g, 70 mmol) in THF (50 mL) was added sodium (27 mg, 1.2 mmol). The mixture was stirred at rt for 2 h. Then tert-butyl acrylate (3.0 g, 23 mmol) was added. The mixture was stirred at rt for 16 h. The reaction was monitored by TLC. Upon completion, the mixture was concentrated, and the residue was purified by silica gel chromatography (EtOAc/petroleum ether=0:1 to 1:1) to provide the title compound (1.8 g, 16% yield) as a colorless oil.

### Step 2. Synthesis of 1-(tert-butyl) 22-methyl 4,7,10,13,16,19-hexaoxadocosanedioate

A mixture of tert-butyl 1-hydroxy-3,6,9,12,15-pentaoxaoctadecan-18-oate (1.8 g, 5 mmol), methyl acrylate (5 mL) and DBU (2.2 g, 10 mmol) was stirred at 50° C. for 48 h. The mixture was concentrated, and the residue was purified by silica gel chromatography (EtOAc/petroleum ether=0:1 to 1:1) to provide the title compound (900 mg, 47% yield) as a colorless oil.

### Step 3. Synthesis of 2-amino-N-(4,5-dimethylthiazol-2-yl)benzamide

To a solution of 1-(tert-butyl) 22-methyl 4,7,10,13,16,19-hexaoxadocosanedioate (800 mg, 1.8 mmol) in DCM (10 mL) was added TFA (2 mL). The mixture was stirred at rt for 6 h. The reaction was monitored by TLC. Upon completion, the mixture was concentrated to afford the title compound (600 mg, 95% yield) as a colorless oil.

### Step 4. Synthesis of methyl 22-((2-((4,5-dimethylthiazol-2-yl)carbamoyl)phenyl)amino)-22-oxo-4,7,10,13,16,19-hexaoxadocosanoate

To a solution of 2-amino-N-(4,5-dimethylthiazol-2-yl)benzamide (350 mg, 0.9 mmol) in DMF (5 mL) were added 3-oxo-2,6,9,12,15,18,21-heptaoxatetracosan-24-oic acid (577 mg, 0.9 mmol), HATU (808 mg, 1.3 mmol) and DIEA (313 mg, 1.8 mmol). The mixture was stirred at 50° C. for 6 h. Upon completion, the mixture was extracted with EtOAc (20 mL×3). The combined organic layers were washed with brine (30 mL), dried over sodium sulfate, filtered and concentrated to dryness. The residue was purified by reverse phase chromatography (0.1% TFA in water and ACN) to provide the title compound (300 mg, 57% yield) as a yellow oil. MS (ESI) m/z=625.8 [M+H]⁺.

### Step 5. Synthesis of 22-((2-((4,5-dimethylthiazol-2-yl)carbamoyl)phenyl)amino)-22-oxo-4,7,10,13,16,19-hexaoxadocosanoic acid

To a solution of methyl 22-((2-((4,5-dimethylthiazol-2-yl)carbamoyl)phenyl)amino)-22-oxo-4,7,10,13,16,19-hexaoxadocosanoate (300 mg, 0.5 mmol) in THF (8 mL) and H₂O (2 mL) was added LiOH (101 mg, 26.2 mmol). The mixture was stirred at rt for 3 h. Upon completion, the mixture was acidified to pH=1-2, and extracted with EtOAc. The organic layer was concentrated to provide the title compound (230 mg, 78% yield) as a white solid. MS (ESI) m/z=611.8 [M+H]⁺.

### Example 301. 22-((2-((4,5-Dimethylthiazol-2-yl)carbamoyl)phenyl)amino)-22-oxo-4,7,10,13,16,19-hexaoxadocosanoic acid (B1-79)

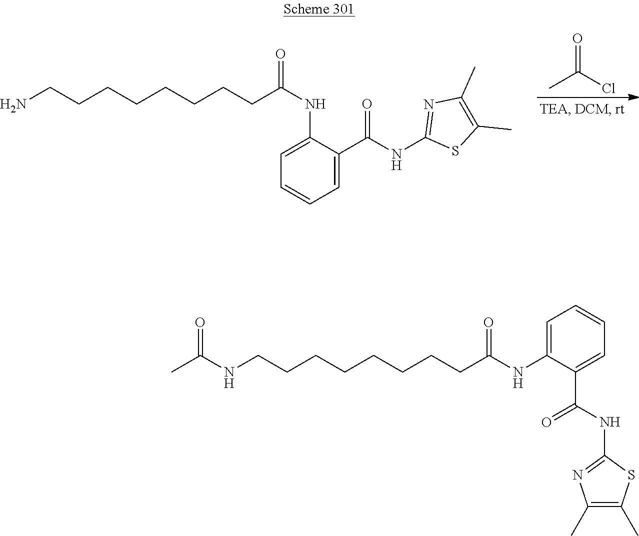

A solution of 2-(9-aminononanamido)-N-(4,5-dimethylthiazol-2-yl)benzamide (5 mg, 12.4 μmol), acetyl chloride (1.5 mg, 18.6 μmol) and TEA (3.8 mg, 37.2 μmol) in DCM (2 mL) was stirred at rt. Upon completion, the mixture was concentrated at rt under reduced pressure. The residue was purified by silica gel chromatography (DCM/MeOH=30:1) to provide the title compound (2.13 mg, 39% yield) as a white solid. MS (ESI) m/z=445.6 [M+H]⁺.

### Example 302. 3-((2-(2-(2-Acetamidoethoxy)ethoxy)ethyl)amino)-N-(4,5-dimethylthiazol-2-yl)-2-methylbenzamide (B1-80)

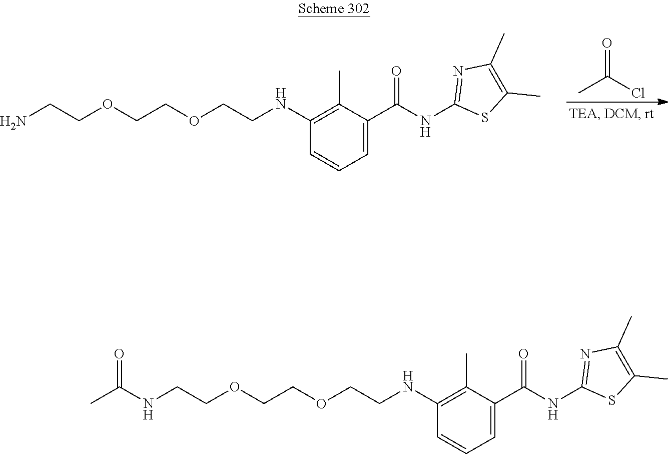

B1-80 was synthesized following the standard procedure for preparing B1-79 (1.01 mg, 18% yield) as a white solid. MS (ESI) m/z=435.6 [M+H]⁺.

### Example 303. 5-((3-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)propyl)amino)-N-(4,5-dimethylthiazol-2-yl)-2-methylbenzamide (CPD-087)

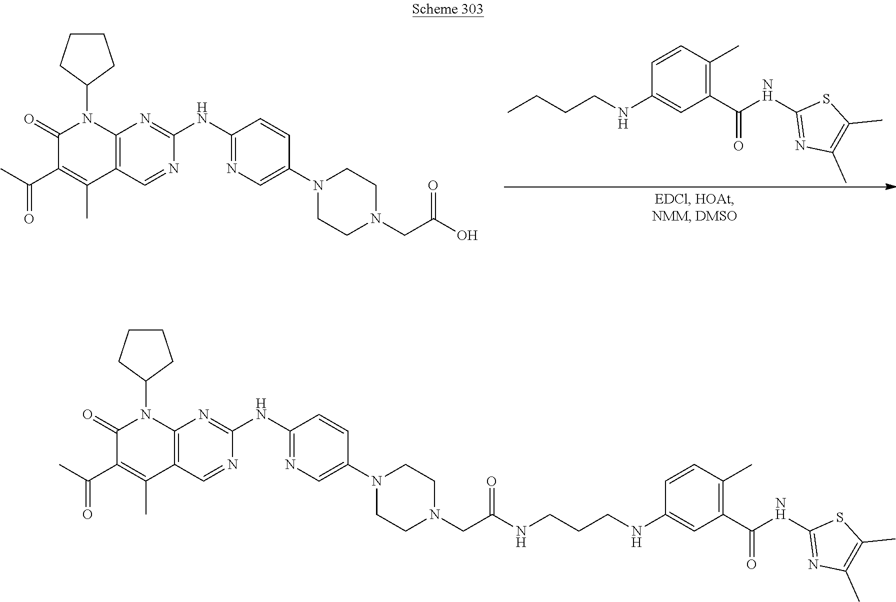 [SMILES: CC(=O)c1c(C)c2cnc(Nc3ccc(N4CCN(CC(=O)O)CC4)cn3)nc2n(C2CCCC2)c1=O ; CCCCNc1ccc(C)c(C(=O)Nc2nc(C)c(C)s2)c1 ; CC(=O)c1c(C)c2cnc(Nc3ccc(N4CCN(CC(=O)NCCCNc5ccc(C)c(C(=O)Nc6nc(C)c(C)s6)c5)CC4)cn3)nc2n(C2CCCC2)c1=O]

CPD-087 was synthesized following the standard procedure for preparing CPD-042 (4.0 mg, 25% yield). MS (ESI) m/z=806.8 [M+H]⁺.

### Example 304. 5-((4-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)butyl)amino)-N-(4,5-dimethylthiazol-2-yl)-2-methylbenzamide (CPD-088)

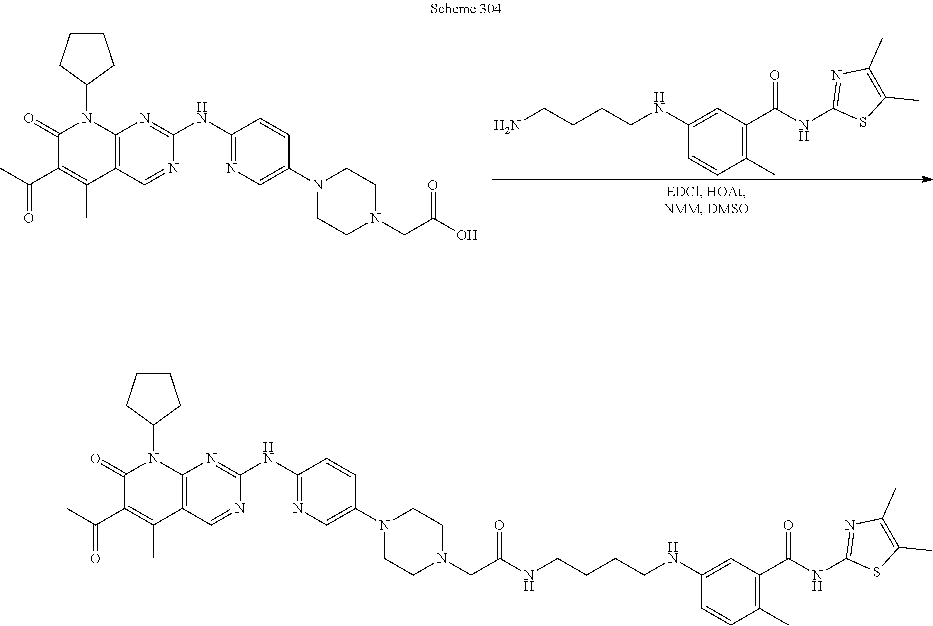 [SMILES: CC(=O)c1c(C)c2cnc(Nc3ccc(N4CCN(CC(=O)O)CC4)cn3)nc2n(C2CCCC2)c1=O ; Cc1ccc(NCCCCN)cc1C(=O)Nc1nc(C)c(C)s1 ; CC(=O)c1c(C)c2cnc(Nc3ccc(N4CCN(CC(=O)NCCCCNc5ccc(C)c(C(=O)Nc6nc(C)c(C)s6)c5)CC4)cn3)nc2n(C2CCCC2)c1=O]

CPD-088 was synthesized following the standard procedure for preparing CPD-042 (3.6 mg, 22% yield). MS (ESI) m/z=820.9 [M+H]⁺.

### Example 305. 5-((2-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)ethyl)amino)-N-(4,5-dimethylthiazol-2-yl)-2-methylbenzamide (CPD-089)

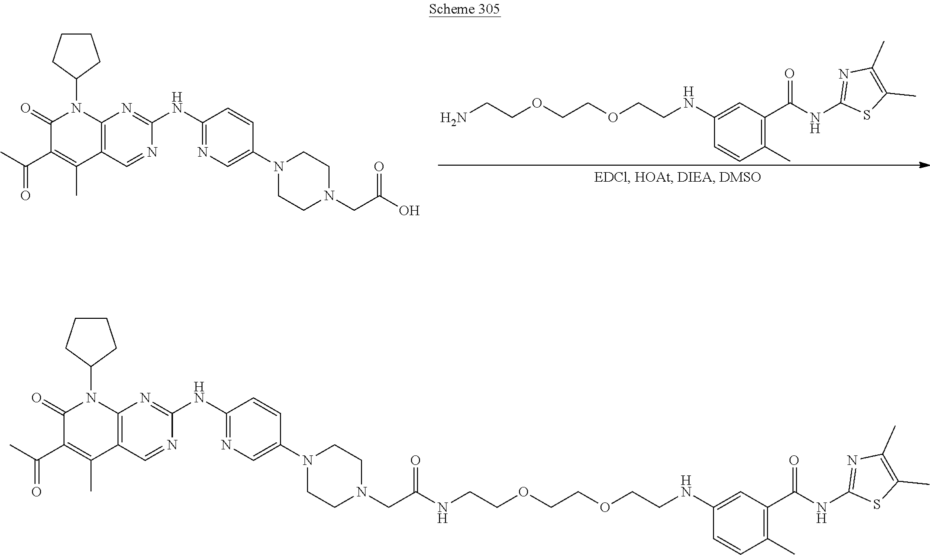 [SMILES: CC(=O)c1c(C)c2cnc(Nc3ccc(N4CCN(CC(=O)O)CC4)cn3)nc2n(C2CCCC2)c1=O ; Cc1ccc(NCCOCCOCCN)cc1C(=O)Nc1nc(C)c(C)s1 ; CC(=O)c1c(C)c2cnc(Nc3ccc(N4CCN(CC(=O)NCCOCCOCCNc5ccc(C)c(C(=O)Nc6nc(C)c(C)s6)c5)CC4)cn3)nc2n(C2CCCC2)c1=O]

CPD-089 was synthesized following the standard procedure for preparing CPD-167 (3.4 mg, 19% yield). MS (ESI) m/z=881.0 [M+H]⁺.

### Example 306. 3-((8-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)octyl)amino)-N-(4,5-dimethylthiazol-2-yl)-2-methylbenzamide (CPD-090)

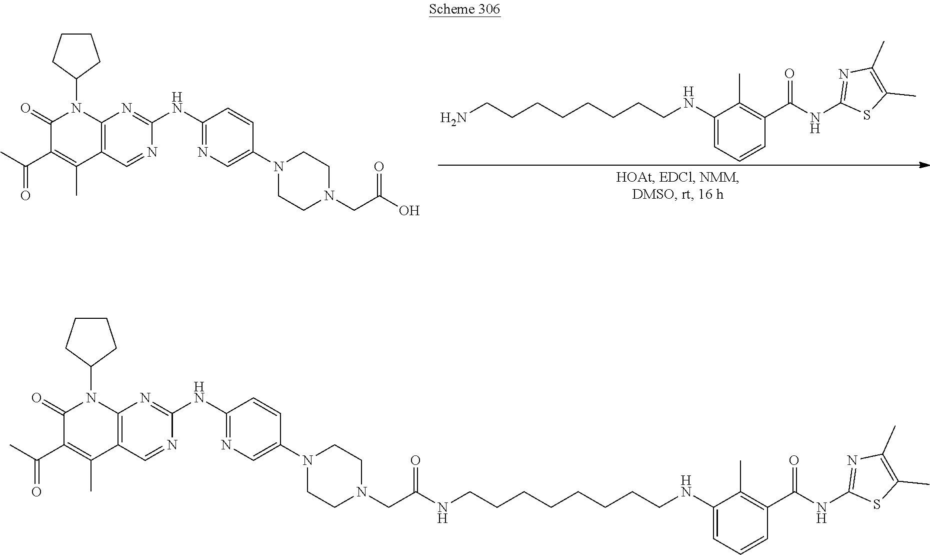 [SMILES: CC(=O)c1c(C)c2cnc(Nc3ccc(N4CCN(CC(=O)O)CC4)cn3)nc2n(C2CCCC2)c1=O ; Cc1nc(NC(=O)c2cccc(NCCCCCCCCN)c2C)sc1C ; CC(=O)c1c(C)c2cnc(Nc3ccc(N4CCN(CC(=O)NCCCCCCCCNc5cccc(C(=O)Nc6nc(C)c(C)s6)c5C)CC4)cn3)nc2n(C2CCCC2)c1=O]

CPD-090 was synthesized following the standard procedure for preparing CPD-042 (TFA salt, 1.3 mg, 1% yield) as yellow solid. MS (ESI) m/z=876.9 [M+H]⁺.

### Example 307. 5-((3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-3-oxopropyl)amino)-N-(4,5-dimethylthiazol-2-yl)-2-methylbenzamide (CPD-091)

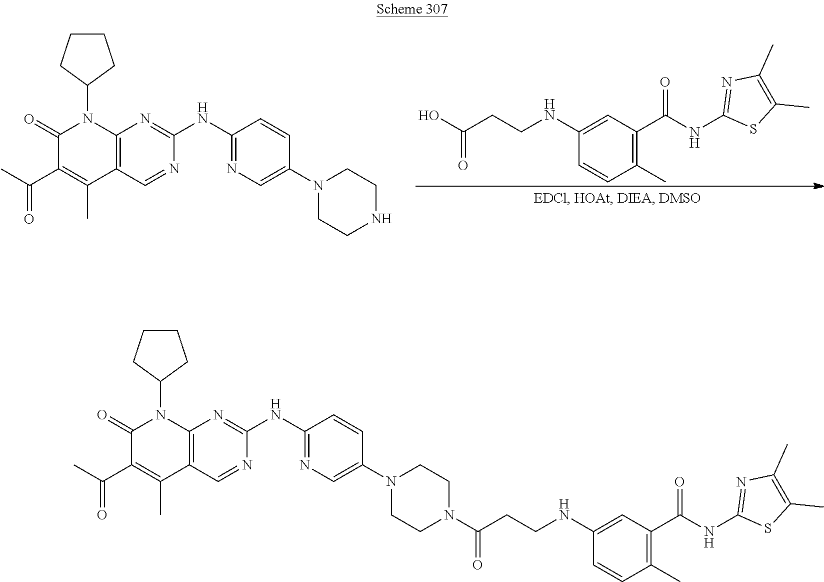 [SMILES: CC(=O)c1c(C)c2cnc(Nc3ccc(N4CCNCC4)cn3)nc2n(C2CCCC2)c1=O ; Cc1ccc(NCCC(=O)O)cc1C(=O)Nc1nc(C)c(C)s1 ; CC(=O)c1c(C)c2cnc(Nc3ccc(N4CCN(C(=O)CCNc5ccc(C)c(C(=O)Nc6nc(C)c(C)s6)c5)CC4)cn3)nc2n(C2CCCC2)c1=O]

CPD-091 was synthesized following the standard procedure for preparing CPD-167 (5.3 mg, 31% yield). MS (ESI) m/z=763.9 [M+H]⁺.

### Example 308. 3-((2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)amino)-N-(4,5-dimethylthiazol-2-yl)-2-methylbenzamide (CPD-092)

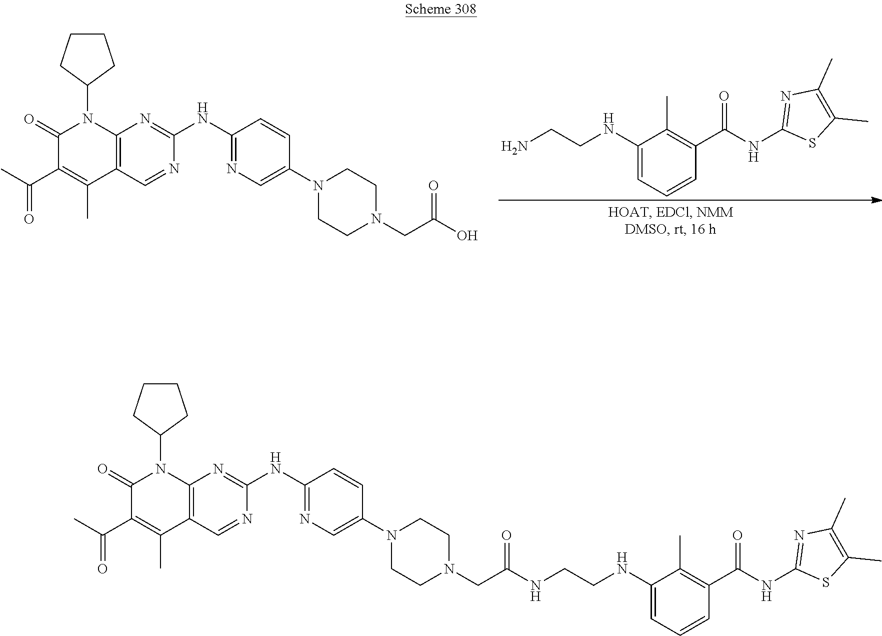 [SMILES: CC(=O)c1c(C)c2cnc(Nc3ccc(N4CCN(CC(=O)O)CC4)cn3)nc2n(C2CCCC2)c1=O ; Cc1nc(NC(=O)c2cccc(NCCN)c2C)sc1C ; CC(=O)c1c(C)c2cnc(Nc3ccc(N4CCN(CC(=O)NCCNc5cccc(C(=O)Nc6nc(C)c(C)s6)c5C)CC4)cn3)nc2n(C2CCCC2)c1=O]

CPD-092 was synthesized following the standard procedure for preparing CPD-042 (TFA salt, 7.55 mg, 7% yield). MS (ESI) m/z=792.9 [M+H]⁺.

### Example 309. 5-((2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)amino)-N-(4,5-dimethylthiazol-2-yl)-2-methylbenzamide (CPD-093)

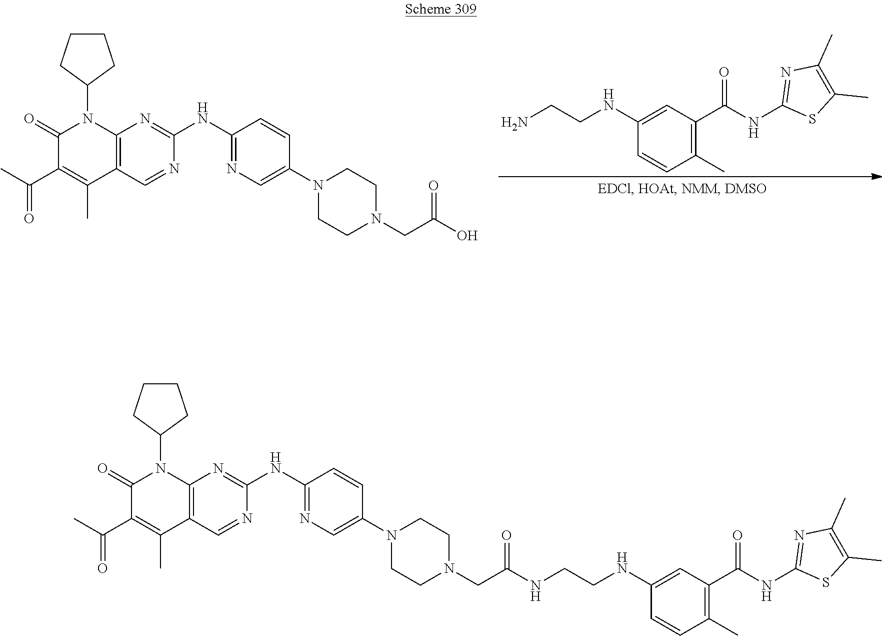 [SMILES: CC(=O)c1c(C)c2cnc(Nc3ccc(N4CCN(CC(=O)O)CC4)cn3)nc2n(C2CCCC2)c1=O ; Cc1ccc(NCCN)cc1C(=O)Nc1nc(C)c(C)s1 ; CC(=O)c1c(C)c2cnc(Nc3ccc(N4CCN(CC(=O)NCCNc5ccc(C)c(C(=O)Nc6nc(C)c(C)s6)c5)CC4)cn3)nc2n(C2CCCC2)c1=O]

CPD-093 was synthesized following the standard procedure for preparing CPD-042 (1.8 mg, 12% yield). MS (ESI) m/z=792.9 [M+H]⁺.

### Example 310. 5-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)hexyl)amino)-N-(4,5-dimethylthiazol-2-yl)-2-methylbenzamide (CPD-094)

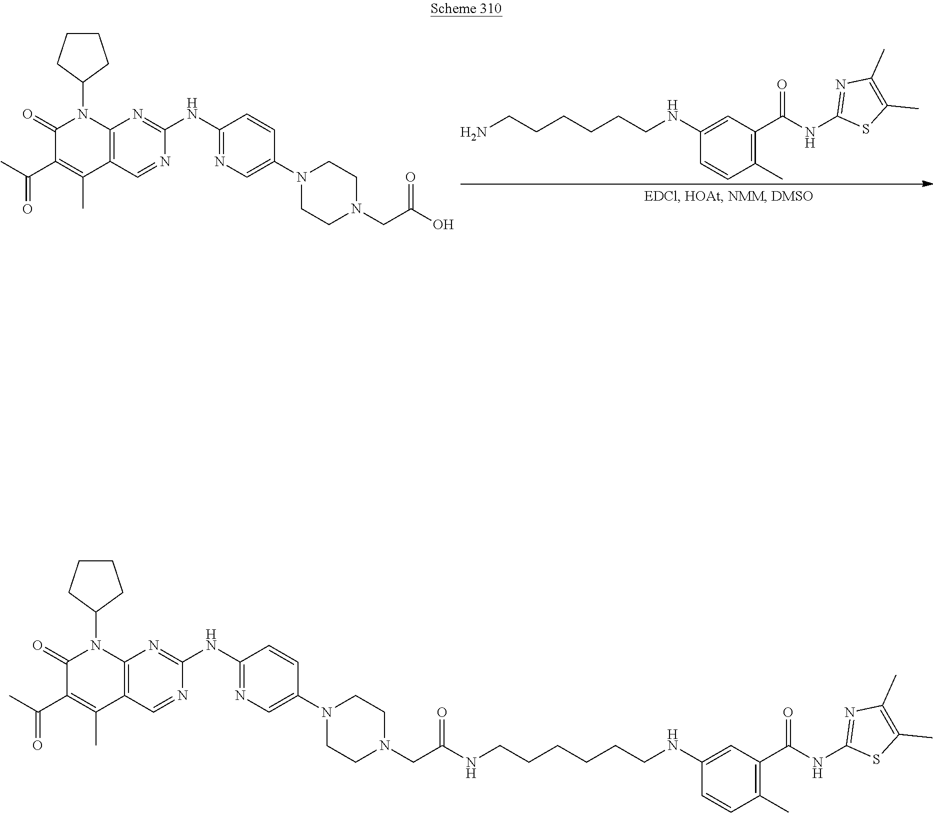 [SMILES: CC(=O)c1c(C)c2cnc(Nc3ccc(N4CCN(CC(=O)O)CC4)cn3)nc2n(C2CCCC2)c1=O ; Cc1ccc(NCCCCCCN)cc1C(=O)Nc1nc(C)c(C)s1 ; CC(=O)c1c(C)c2cnc(Nc3ccc(N4CCN(CC(=O)NCCCCCCNc5ccc(C)c(C(=O)Nc6nc(C)c(C)s6)c5)CC4)cn3)nc2n(C2CCCC2)c1=O]

CPD-094 was synthesized following the standard procedure for preparing CPD-042 (2.6 mg, 16% yield). MS (ESI) m/z=848.9 [M+H]⁺.

### Example 311. 5-((8-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)octyl)amino)-N-(4,5-dimethylthiazol-2-yl)-2-methylbenzamide (CPD-095)

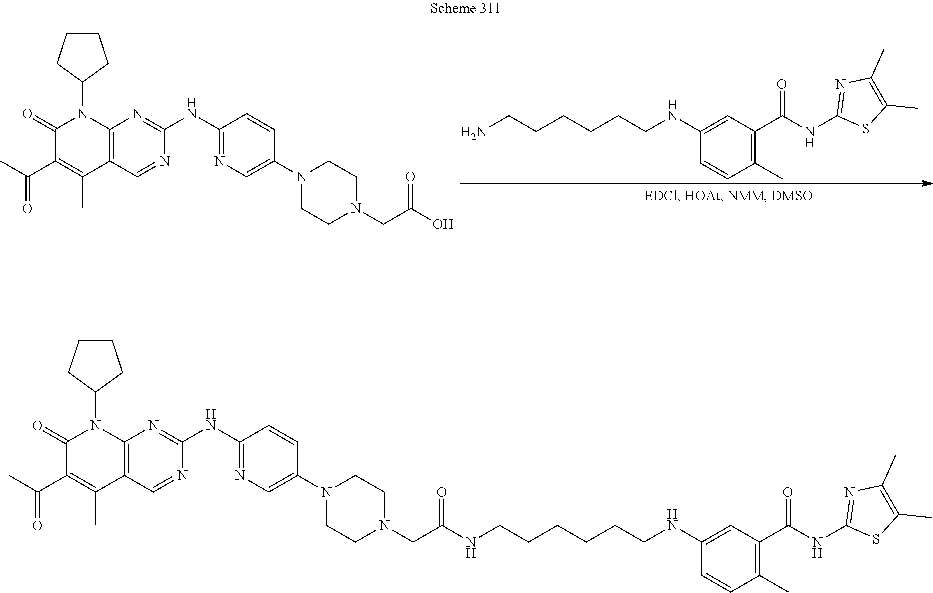 [SMILES: CC(=O)c1c(C)c2cnc(Nc3ccc(N4CCN(CC(=O)O)CC4)cn3)nc2n(C2CCCC2)c1=O ; Cc1ccc(NCCCCCCN)cc1C(=O)Nc1nc(C)c(C)s1 ; CC(=O)c1c(C)c2cnc(Nc3ccc(N4CCN(CC(=O)NCCCCCCNc5ccc(C)c(C(=O)Nc6nc(C)c(C)s6)c5)CC4)cn3)nc2n(C2CCCC2)c1=O]

CPD-095 was synthesized following the standard procedure for preparing CPD-042 (4.8 mg, 28% yield). MS (ESI) m/z=877.0 [M+H]⁺.

### Example 312. 5-((2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethyl)amino)-N-(4,5-dimethylthiazol-2-yl)-2-methylbenzamide (CPD-096)

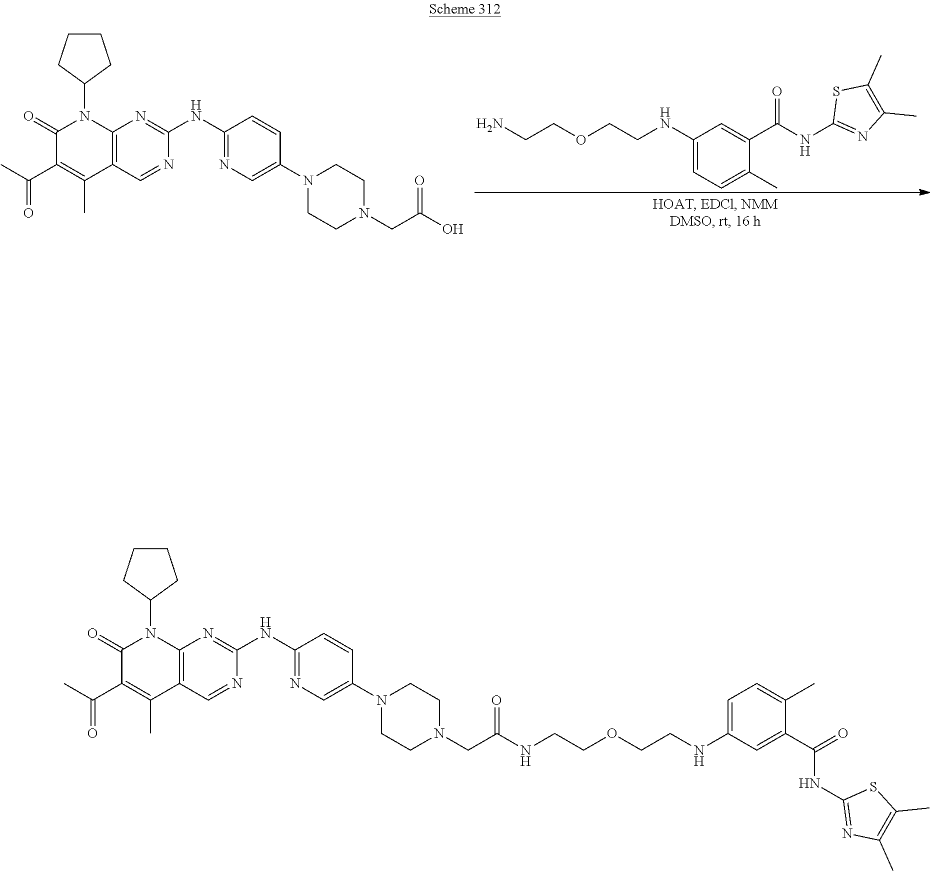 [SMILES: CC(=O)c1c(C)c2cnc(Nc3ccc(N4CCN(CC(=O)O)CC4)cn3)nc2n(C2CCCC2)c1=O ; Cc1ccc(NCCOCCN)cc1C(=O)Nc1nc(C)c(C)s1 ; CC(=O)c1c(C)c2cnc(Nc3ccc(N4CCN(CC(=O)NCCOCCNc5ccc(C)c(C(=O)Nc6nc(C)c(C)s6)c5)CC4)cn3)nc2n(C2CCCC2)c1=O]

CPD-096 was synthesized following the standard procedure for preparing CPD-042 (TFA salt, 3.93 mg, 2% yield). MS (ESI) m/z=837.0 [M+H]⁺.

### Example 313. 5-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-N-(4,5-dimethylthiazol-2-yl)-2-methylbenzamide (CPD-097)

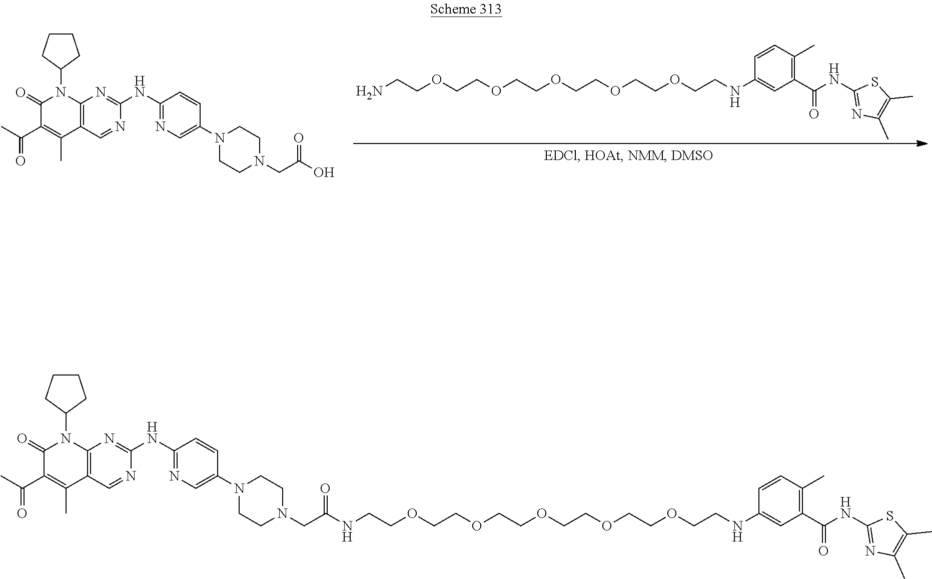 [SMILES: CC(=O)c1c(C)c2cnc(Nc3ccc(N4CCN(CC(=O)O)CC4)cn3)nc2n(C2CCCC2)c1=O ; Cc1ccc(NCCOCCOCCOCCOCCOCCN)cc1C(=O)Nc1nc(C)c(C)s1 ; CC(=O)c1c(C)c2cnc(Nc3ccc(N4CCN(CC(=O)NCCOCCOCCOCCOCCOCCNc5ccc(C)c(C(=O)Nc6nc(C)c(C)s6)c5)CC4)cn3)nc2n(C2CCCC2)c1=O]

CPD-097 was synthesized following the standard procedure for preparing CPD-042 (1.0 mg, 5% yield). MS (ESI) m/z=1013.2 [M+H]⁺.

### Example 314. 3-((3-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)propyl)amino)-N-(4,5-dimethylthiazol-2-yl)-2-methylbenzamide (CPD-098)

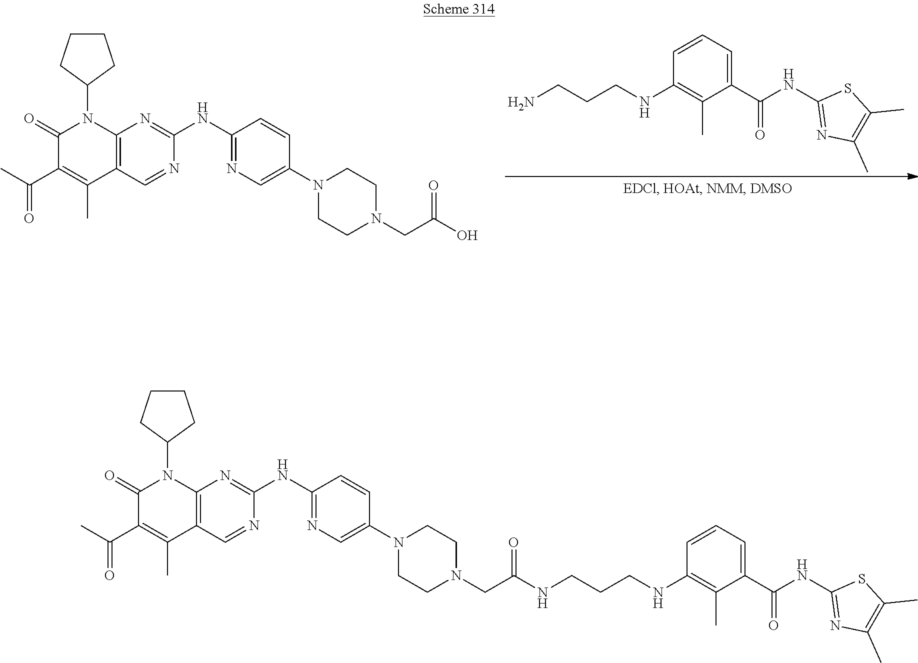 [SMILES: CC(=O)c1c(C)c2cnc(Nc3ccc(N4CCN(CC(=O)O)CC4)cn3)nc2n(C2CCCC2)c1=O ; Cc1nc(NC(=O)c2cccc(NCCCN)c2C)sc1C ; CC(=O)c1c(C)c2cnc(Nc3ccc(N4CCN(CC(=O)NCCCNc5cccc(C(=O)Nc6nc(C)c(C)s6)c5C)CC4)cn3)nc2n(C2CCCC2)c1=O]

CPD-098 was synthesized following the standard procedure for preparing CPD-042 (6.25 mg, 26% yield). MS (ESI) m/z=806.9 [M+H]⁺.

### Example 315. 3-((4-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)butyl)amino)-N-(4,5-dimethylthiazol-2-yl)-2-methylbenzamide (CPD-099)

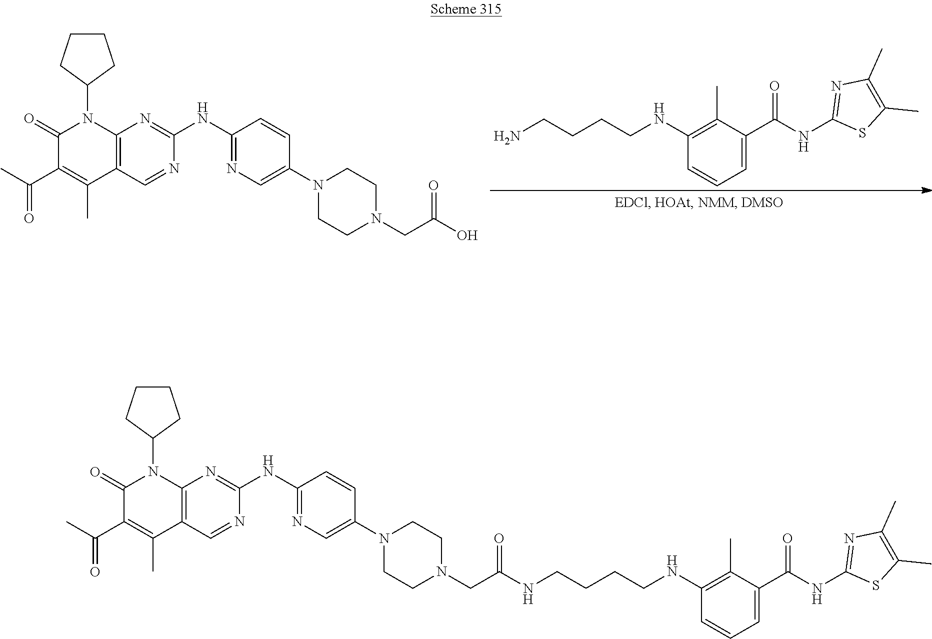 [SMILES: CC(=O)c1c(C)c2cnc(Nc3ccc(N4CCN(CC(=O)O)CC4)cn3)nc2n(C2CCCC2)c1=O ; Cc1nc(NC(=O)c2cccc(NCCCCN)c2C)sc1C ; CC(=O)c1c(C)c2cnc(Nc3ccc(N4CCN(CC(=O)NCCCCNc5cccc(C(=O)Nc6nc(C)c(C)s6)c5C)CC4)cn3)nc2n(C2CCCC2)c1=O]

CPD-099 was synthesized following the standard procedure for preparing CPD-042 (9.11 mg, 37% yield). MS (ESI) m/z=820.9 [M+H]⁺.

### Example 316. 3-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(4,5-dimethylthiazol-2-yl)-2-methylbenzamide (CPD-100)

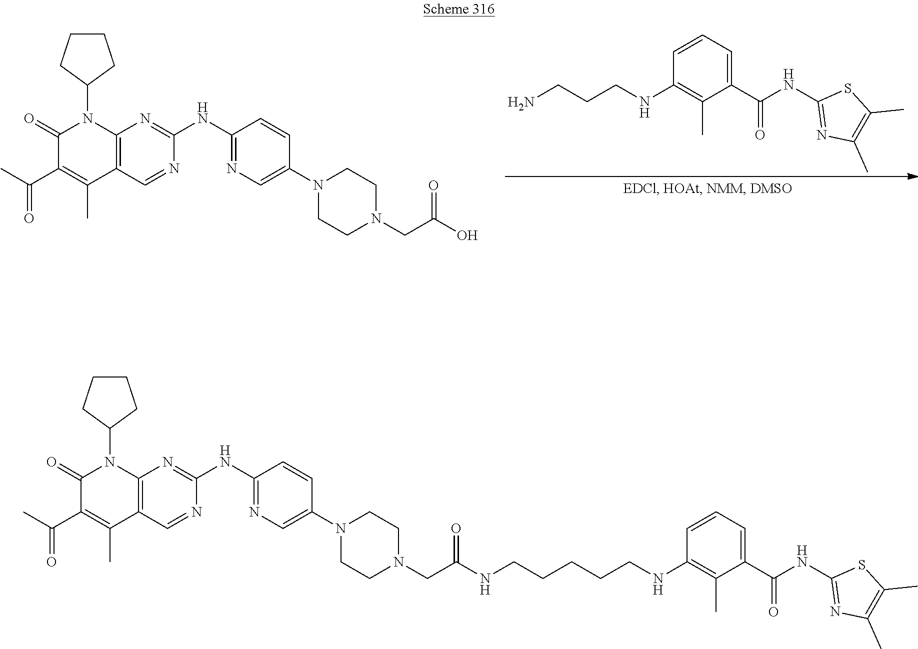 [SMILES: CC(=O)c1c(C)c2cnc(Nc3ccc(N4CCN(CC(=O)O)CC4)cn3)nc2n(C2CCCC2)c1=O ; Cc1nc(NC(=O)c2cccc(NCCCN)c2C)sc1C ; CC(=O)c1c(C)c2cnc(Nc3ccc(N4CCN(CC(=O)NCCCCCNc5cccc(C(=O)Nc6nc(C)c(C)s6)c5C)CC4)cn3)nc2n(C2CCCC2)c1=O]

CPD-100 was synthesized following the standard procedure for preparing CPD-042 (12.23 mg, 49% yield). MS (ESI) m/z=834.8 [M+H]⁺.

### Example 317. 3-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)hexyl)amino)-N-(4,5-dimethylthiazol-2-yl)-2-methylbenzamide (CPD-101)

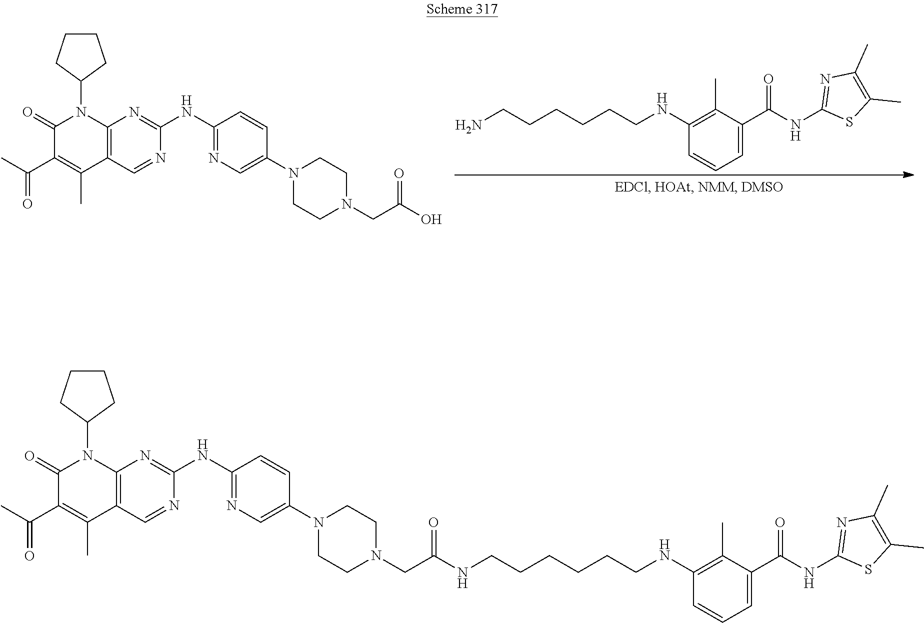 [SMILES: CC(=O)c1c(C)c2cnc(Nc3ccc(N4CCN(CC(=O)O)CC4)cn3)nc2n(C2CCCC2)c1=O ; Cc1nc(NC(=O)c2cccc(NCCCCCCN)c2C)sc1C ; CC(=O)c1c(C)c2cnc(Nc3ccc(N4CCN(CC(=O)NCCCCCCNc5cccc(C(=O)Nc6nc(C)c(C)s6)c5C)CC4)cn3)nc2n(C2CCCC2)c1=O]

CPD-101 was synthesized following the standard procedure for preparing CPD-042 (12.80 mg, 50% yield). MS (ESI) m/z=849.0 [M+H]⁺.

### Example 318. 3-((7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(4,5-dimethylthiazol-2-yl)-2-methylbenzamide (CPD-102)

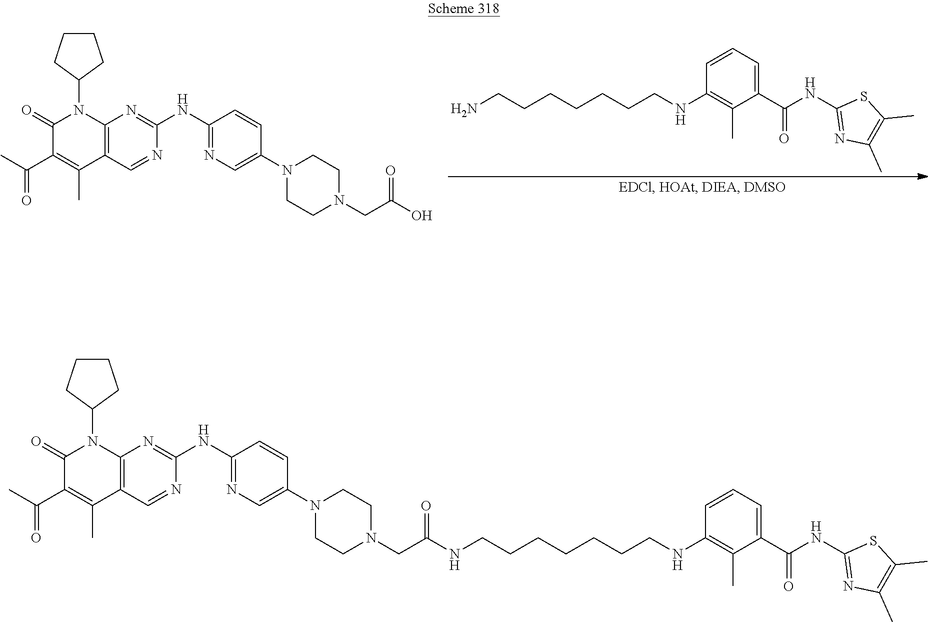 [SMILES: CC(=O)c1c(C)c2cnc(Nc3ccc(N4CCN(CC(=O)O)CC4)cn3)nc2n(C2CCCC2)c1=O ; Cc1nc(NC(=O)c2cccc(NCCCCCCCN)c2C)sc1C ; CC(=O)c1c(C)c2cnc(Nc3ccc(N4CCN(CC(=O)NCCCCCCCNc5cccc(C(=O)Nc6nc(C)c(C)s6)c5C)CC4)cn3)nc2n(C2CCCC2)c1=O]

CPD-102 was synthesized following the standard procedure for preparing CPD-167 (3.7 mg, 21% yield). MS (ESI) m/z=862.9 [M+H]⁺.

### Example 319. 3-((2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethyl)amino)-N-(4,5-dimethylthiazol-2-yl)-2-methylbenzamide (CPD-103)

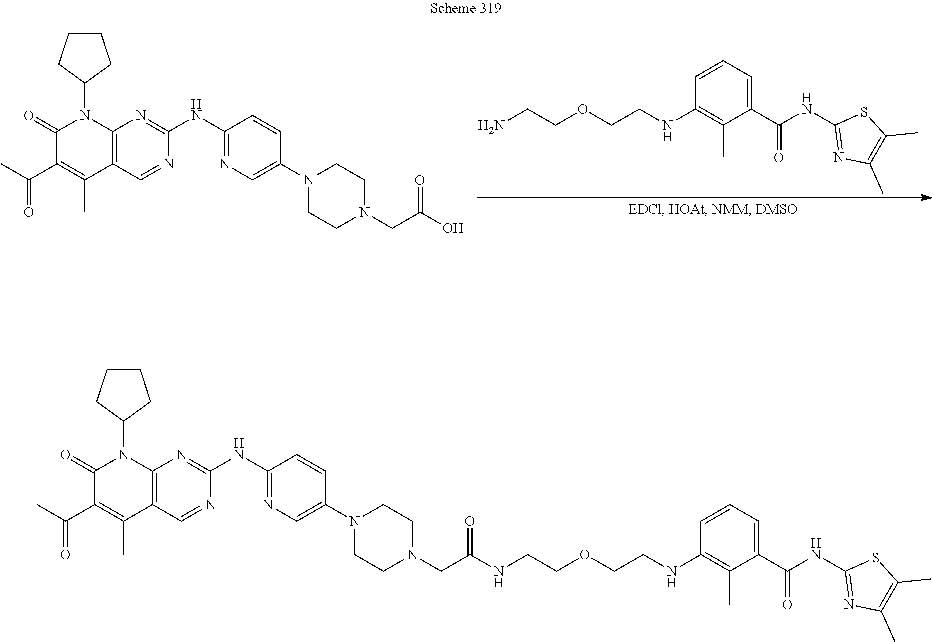 [SMILES: CC(=O)c1c(C)c2cnc(Nc3ccc(N4CCN(CC(=O)O)CC4)cn3)nc2n(C2CCCC2)c1=O ; Cc1nc(NC(=O)c2cccc(NCCOCCN)c2C)sc1C ; CC(=O)c1c(C)c2cnc(Nc3ccc(N4CCN(CC(=O)NCCOCCNc5cccc(C(=O)Nc6nc(C)c(C)s6)c5C)CC4)cn3)nc2n(C2CCCC2)c1=O]

CPD-103 was synthesized following the standard procedure for preparing CPD-042 (3.5 mg, 22% yield). MS (ESI) m/z=836.9 [M+H]⁺.

### Example 320. 3-((2-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)ethyl)amino)-N-(4,5-dimethylthiazol-2-yl)-2-methylbenzamide (CPD-104)

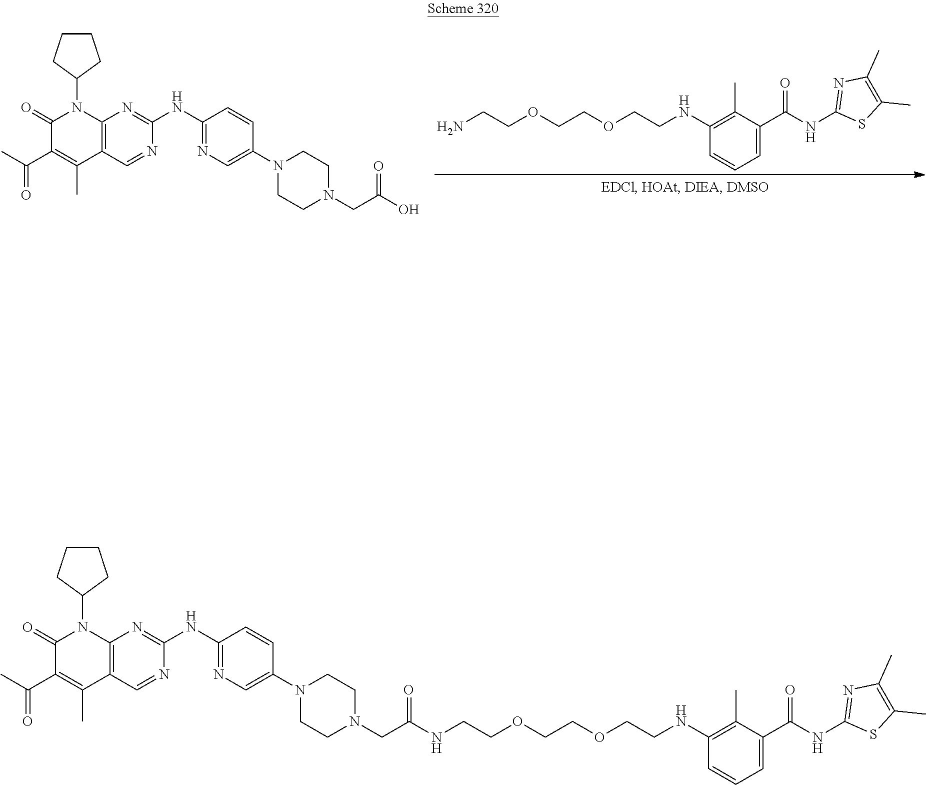 [SMILES: CC(=O)c1c(C)c2cnc(Nc3ccc(N4CCN(CC(=O)O)CC4)cn3)nc2n(C2CCCC2)c1=O ; Cc1nc(NC(=O)c2cccc(NCCOCCOCCN)c2C)sc1C ; CC(=O)c1c(C)c2cnc(Nc3ccc(N4CCN(CC(=O)NCCOCCOCCNc5cccc(C(=O)Nc6nc(C)c(C)s6)c5C)CC4)cn3)nc2n(C2CCCC2)c1=O]

CPD-104 was synthesized following the standard procedure for preparing CPD-167 (3.6 mg, 21% yield). MS (ESI) m/z=881.0 [M+H]⁺.

### Example 321. 3-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12-trioxa-3-azatetradecan-14-yl)amino)-N-(4,5-dimethylthiazol-2-yl)-2-methylbenzamide (CPD-105)

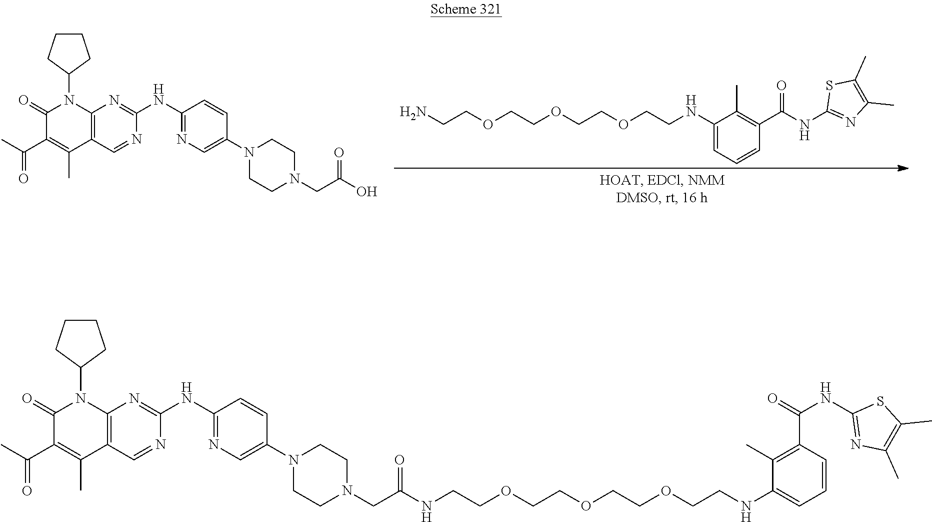 [SMILES: CC(=O)c1c(C)c2cnc(Nc3ccc(N4CCN(CC(=O)O)CC4)cn3)nc2n(C2CCCC2)c1=O ; Cc1nc(NC(=O)c2cccc(NCCOCCOCCOCCN)c2C)sc1C ; CC(=O)c1c(C)c2cnc(Nc3ccc(N4CCN(CC(=O)NCCOCCOCCOCCNc5cccc(C(=O)Nc6nc(C)c(C)s6)c5C)CC4)cn3)nc2n(C2CCCC2)c1=O]

CPD-105 was synthesized following the standard procedure for preparing CPD-042 (TFA salt, 3.51 mg, 2% yield) as a yellow solid. MS (ESI) m/z=925.1 [M+H]⁺.

### Example 322. 3-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15-tetraoxa-3-azaheptadecan-17-yl)amino)-N-(4,5-dimethylthiazol-2-yl)-2-methylbenzamide (CPD-106)

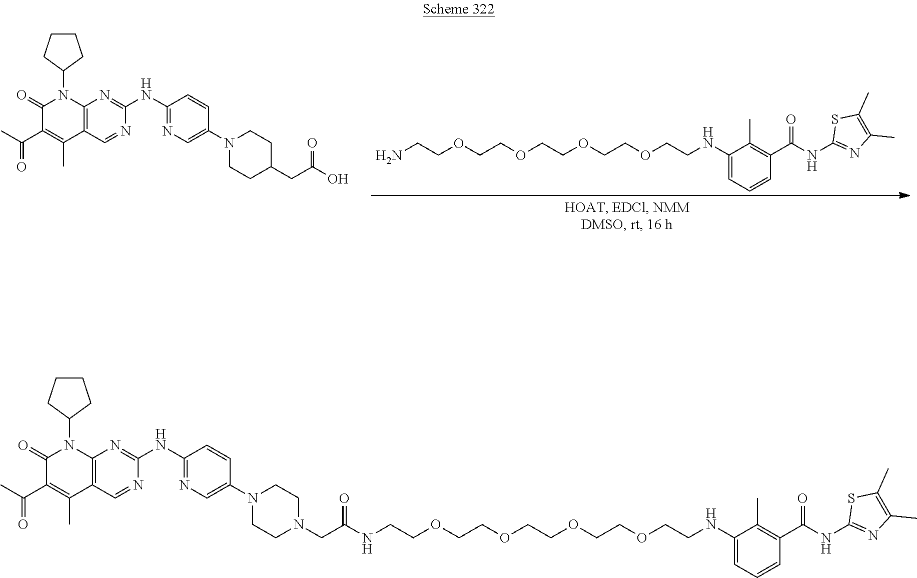 [SMILES: CC(=O)c1c(C)c2cnc(Nc3ccc(N4CCC(CC(=O)O)CC4)cn3)nc2n(C2CCCC2)c1=O ; Cc1nc(NC(=O)c2cccc(NCCOCCOCCOCCOCCN)c2C)sc1C ; CC(=O)c1c(C)c2cnc(Nc3ccc(N4CCN(CC(=O)NCCOCCOCCOCCOCCNc5cccc(C(=O)Nc6nc(C)c(C)s6)c5C)CC4)cn3)nc2n(C2CCCC2)c1=O]

CPD-106 was synthesized following the standard procedure for preparing CPD-042 (TFA salt, 5.49 mg, 3% yield) as a yellow solid. MS (ESI) m/z=969.1 [M+H]⁺.

### Example 323. 3-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azaicosan-20-yl)amino)-N-(4,5-dimethylthiazol-2-yl)-2-methylbenzamide (CPD-107)

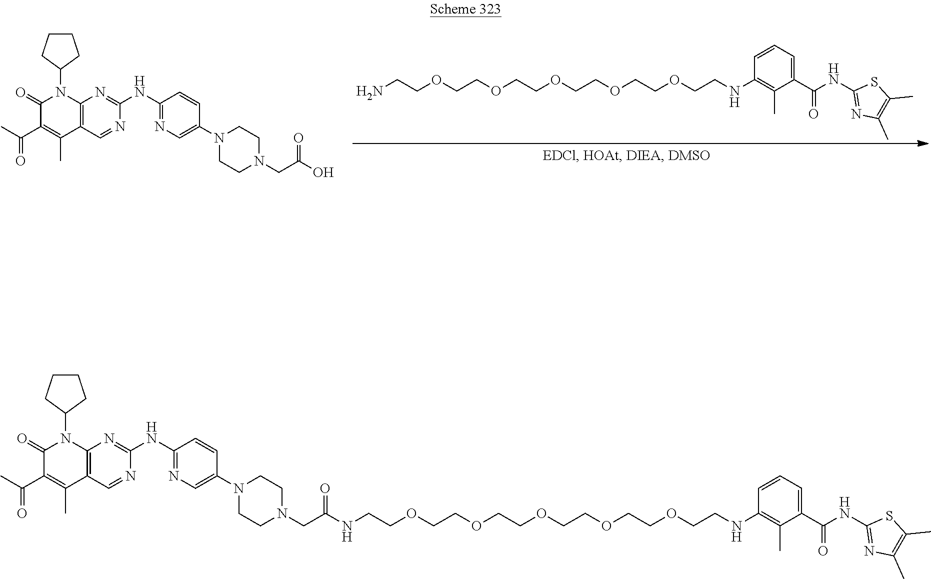 [SMILES: CC(=O)c1c(C)c2cnc(Nc3ccc(N4CCN(CC(=O)O)CC4)cn3)nc2n(C2CCCC2)c1=O ; Cc1nc(NC(=O)c2cccc(NCCOCCOCCOCCOCCOCCN)c2C)sc1C ; CC(=O)c1c(C)c2cnc(Nc3ccc(N4CCN(CC(=O)NCCOCCOCCOCCOCCOCCNc5cccc(C(=O)Nc6nc(C)c(C)s6)c5C)CC4)cn3)nc2n(C2CCCC2)c1=O]

CPD-107 was synthesized following the standard procedure for preparing CPD-167 (2.9 mg, 14% yield). MS (ESI) m/z=1013.1 [M+H]⁺.

### Example 324. 5-((5-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-5-oxopentyl)amino)-N-(4,5-dimethylthiazol-2-yl)-2-methylbenzamide (CPD-108)

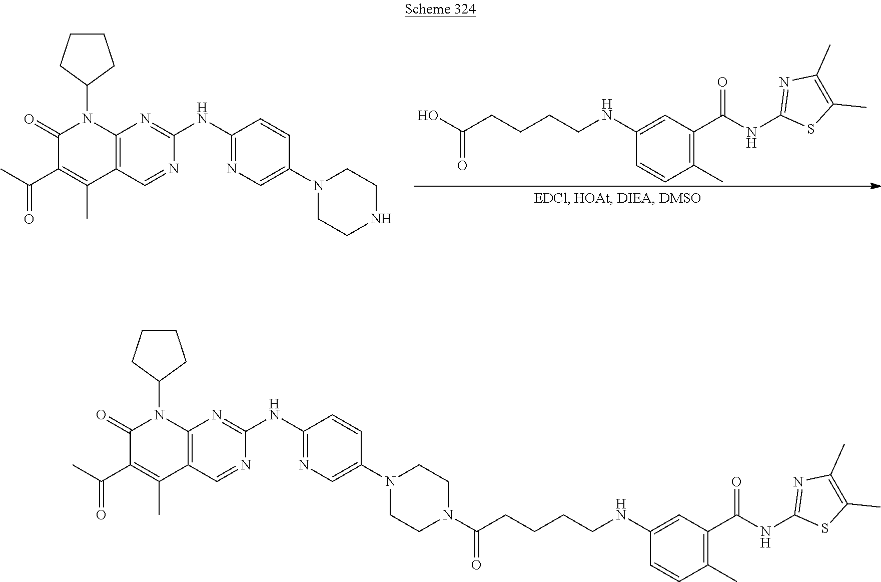 [SMILES: CC(=O)c1c(C)c2cnc(Nc3ccc(N4CCNCC4)cn3)nc2n(C2CCCC2)c1=O ; Cc1ccc(NCCCCC(=O)O)cc1C(=O)Nc1nc(C)c(C)s1 ; CC(=O)c1c(C)c2cnc(Nc3ccc(N4CCN(C(=O)CCCCNc5ccc(C)c(C(=O)Nc6nc(C)c(C)s6)c5)CC4)cn3)nc2n(C2CCCC2)c1=O]

CPD-108 was synthesized following the standard procedure for preparing CPD-167 (0.86 mg, 6% yield). MS (ESI) m/z=791.8 [M+H]⁺.

### Example 325. 5-((7-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-7-oxoheptyl)amino)-N-(4,5-dimethylthiazol-2-yl)-2-methylbenzamide (CPD-109)

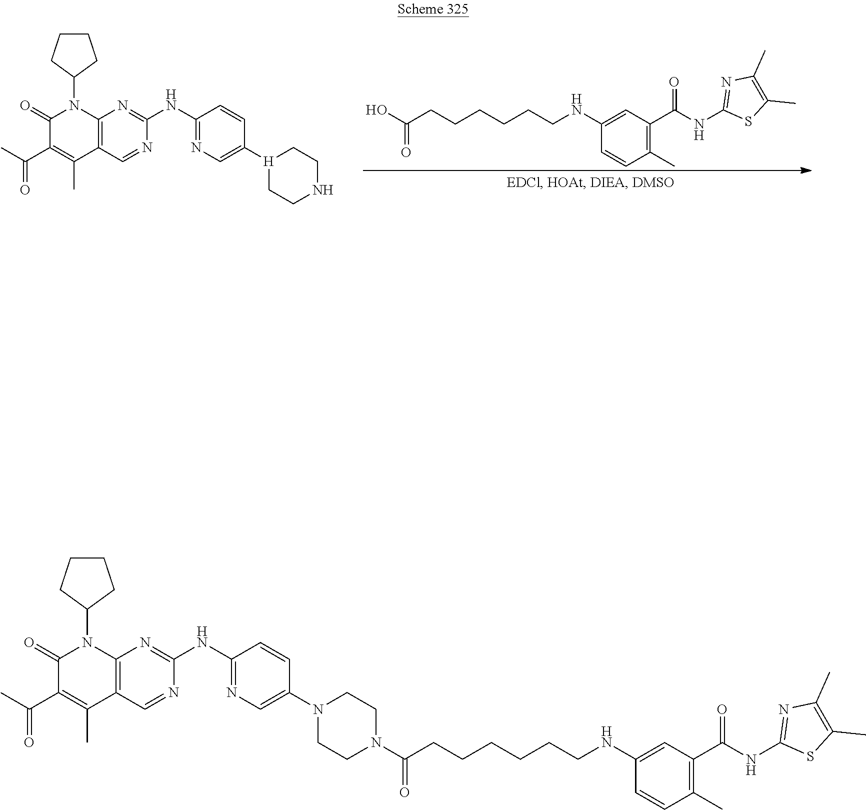

CPD-109 was synthesized following the standard procedure for preparing CPD-167 (1.95 mg, 10% yield). MS (ESI) m/z=819.9 [M+H]⁺.

### Example 326. 5-((2-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-3-oxopropoxy)ethyl)amino)-N-(4,5-dimethylthiazol-2-yl)-2-methylbenzamide (CPD-110)

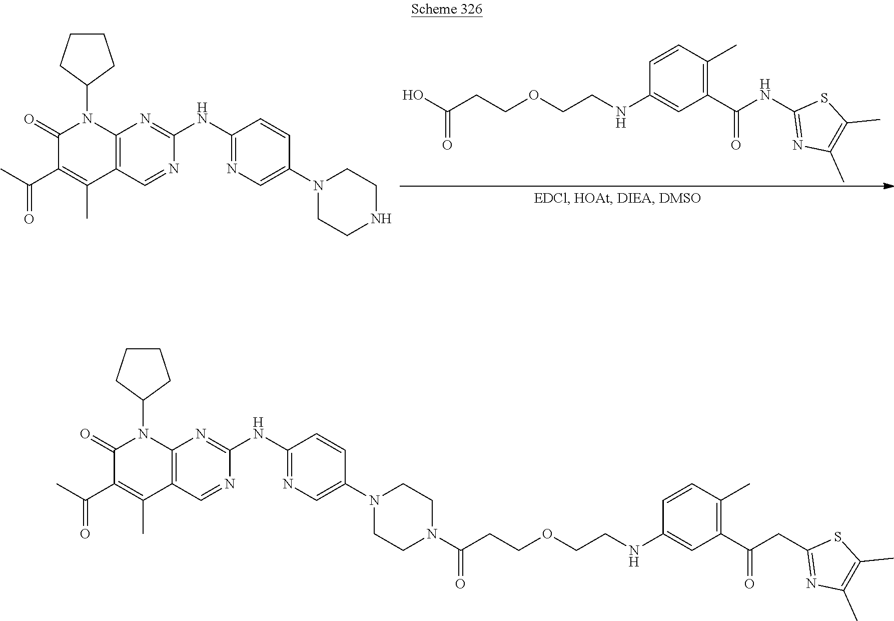 [SMILES: CC(=O)c1c(C)c2cnc(Nc3ccc(N4CCNCC4)cn3)nc2n(C2CCCC2)c1=O ; Cc1ccc(NCCOCCC(=O)O)cc1C(=O)Nc1nc(C)c(C)s1 ; CC(=O)c1c(C)c2cnc(Nc3ccc(N4CCN(C(=O)CCOCCNc5ccc(C)c(C(=O)Cc6nc(C)c(C)s6)c5)CC4)cn3)nc2n(C2CCCC2)c1=O]

CPD-110 was synthesized following the standard procedure for preparing CPD-042 (6.9 mg, 38% yield). MS (ESI) m/z=807.9 [M+H]⁺.

### Example 327. 5-((2-(2-(2-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-3-oxopropoxy)ethoxy)ethoxy)ethyl)amino)-N-(4,5-dimethylthiazol-2-yl)-2-methylbenzamide (CPD-111)

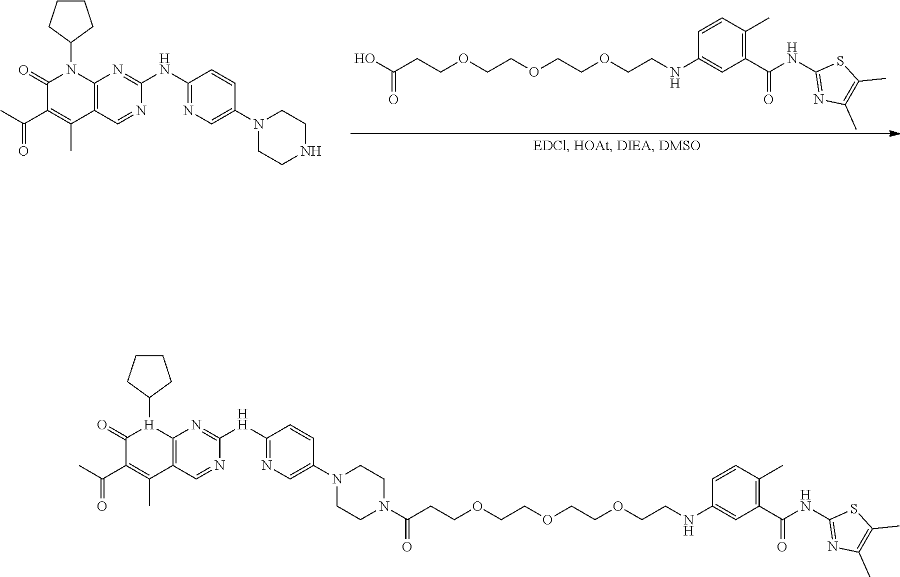

CPD-111 was synthesized following the standard procedure for preparing CPD-167 (6.3 mg, 31% yield). MS (ESI) m/z=895.9 [M+H]⁺.

### Example 328. 3-((3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-3-oxopropyl)amino)-N-(4,5-dimethylthiazol-2-yl)-2-methylbenzamide (CPD-112)

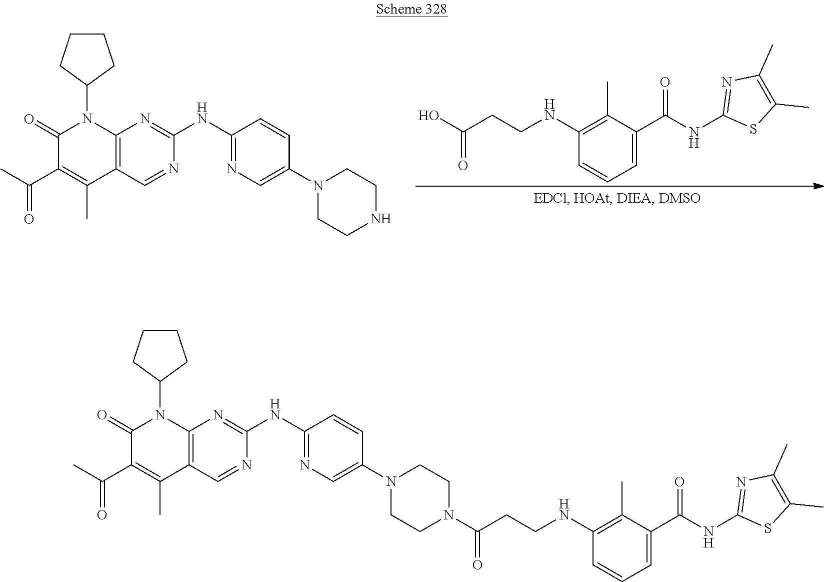 [SMILES: CC(=O)c1c(C)c2cnc(Nc3ccc(N4CCNCC4)cn3)nc2n(C2CCCC2)c1=O ; Cc1nc(NC(=O)c2cccc(NCCC(=O)O)c2C)sc1C ; CC(=O)c1c(C)c2cnc(Nc3ccc(N4CCN(C(=O)CCNc5cccc(C(=O)Nc6nc(C)c(C)s6)c5C)CC4)cn3)nc2n(C2CCCC2)c1=O]

CPD-112 was synthesized following the standard procedure for preparing CPD-042 (2.7 mg, 16% yield). MS (ESI) m/z=763.8 [M+H]⁺.

### Example 329. 2-(9-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)nonanamido)-N-(4,5-dimethylthiazol-2-yl)benzamide (CPD-113)

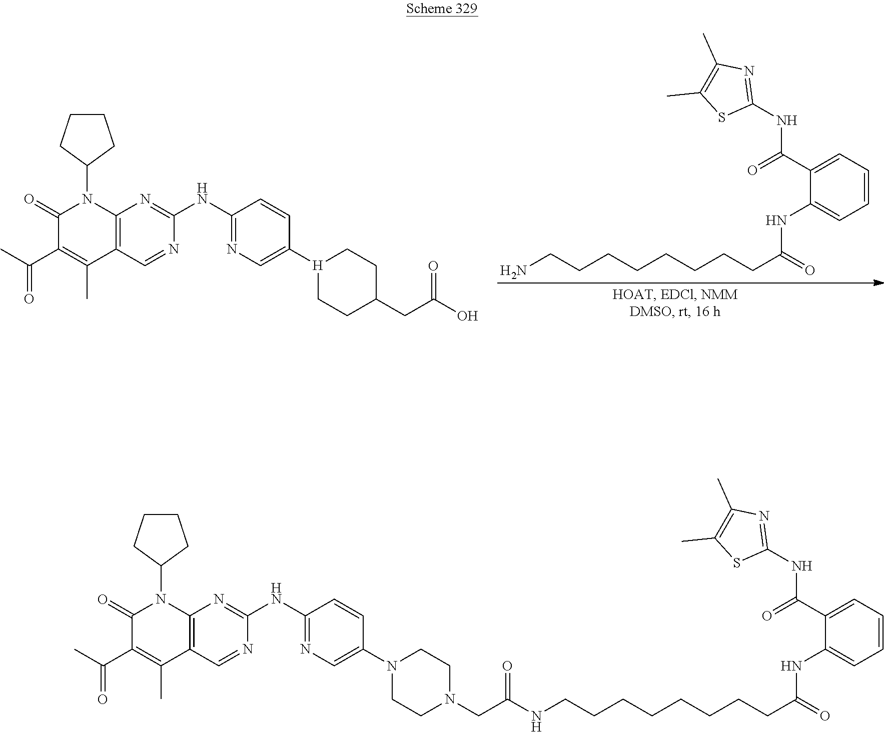

CPD-113 was synthesized following the standard procedure for preparing CPD-042 (TFA salt, 6.62 mg, 4% yield) as a yellow solid. MS (ESI) m/z=890.9 [M+H]⁺.

### Example 330. 2-(1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12-trioxa-3-azapentadecan-15-amido)-N-(4,5-dimethylthiazol-2-yl)benzamide (CPD-114)

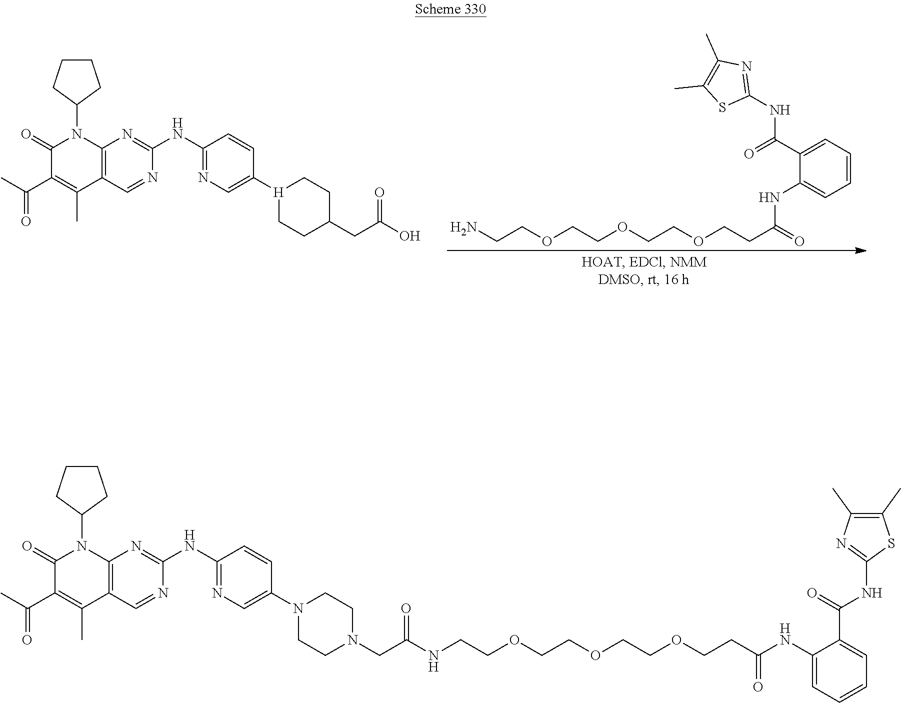

CPD-114 was synthesized following the standard procedure for preparing CPD-042 (TFA salt, 5.65 mg, 3% yield) as a yellow solid. MS (ESI) m/z=939.0 [M+H]⁺.

### Example 331. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4,5-dimethylthiazol-2-yl)benzamide (CPD-115)

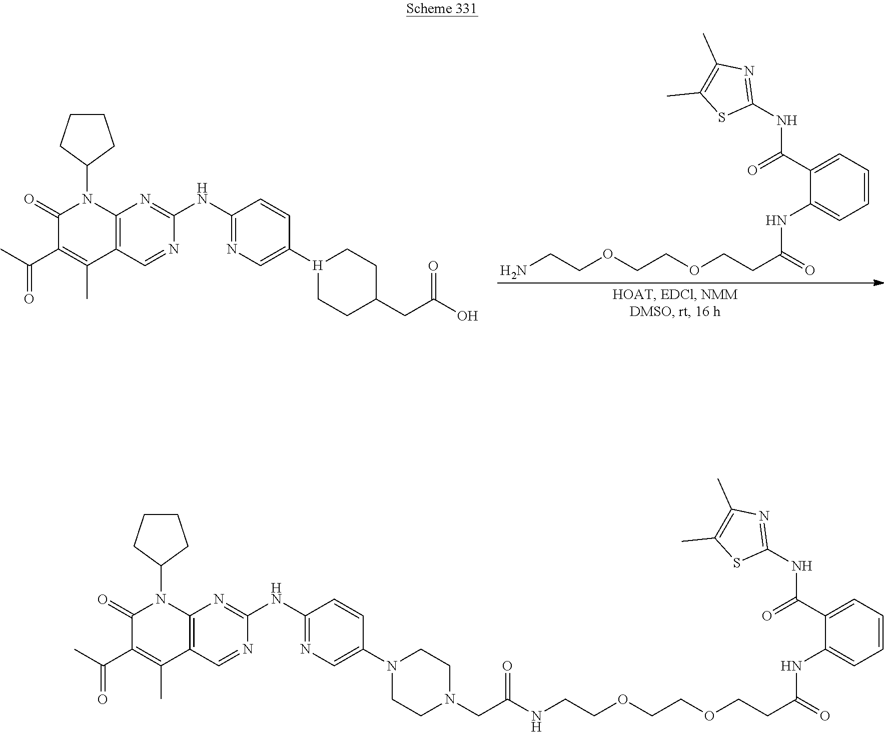

CPD-115 was synthesized following the standard procedure for preparing CPD-042 (TFA salt, 11.74 mg, 7% yield) as a yellow solid. MS (ESI) m/z=895.0 [M+H]⁺.

### Example 332. 5-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(4,5-dimethylthiazol-2-yl)-2-methylbenzamide (CPD-116)

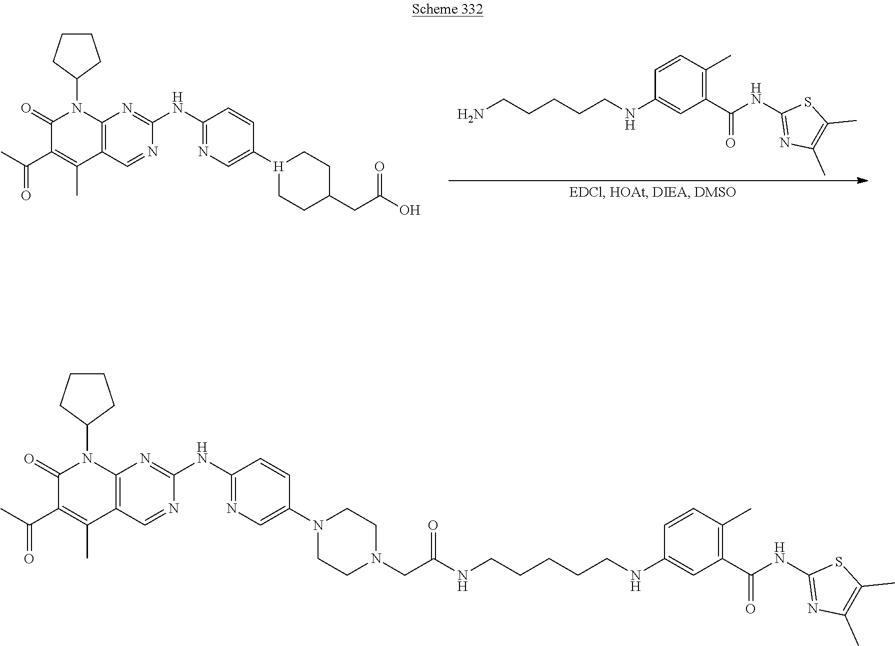

CPD-116 was synthesized following the standard procedure for preparing CPD-167 (3.0 mg, 18% yield). MS (ESI) m/z=834.9 [M+H]⁺.

### Example 333. 5-((7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(4,5-dimethylthiazol-2-yl)-2-methylbenzamide (CPD-117)

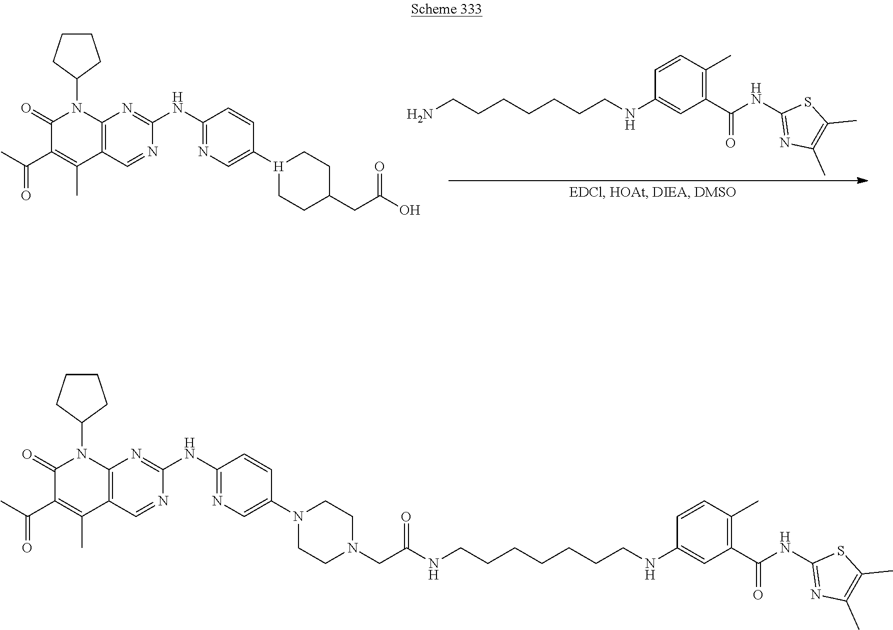

CPD-117 was synthesized following the standard procedure for preparing CPD-167 (2.5 mg, 13% yield). MS (ESI) m/z=862.9 [M+H]⁺.

### Example 334. 5-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15-tetraoxa-3-azaheptadecan-17-yl)amino)-N-(4,5-dimethylthiazol-2-yl)-2-methylbenzamide (CPD-118)

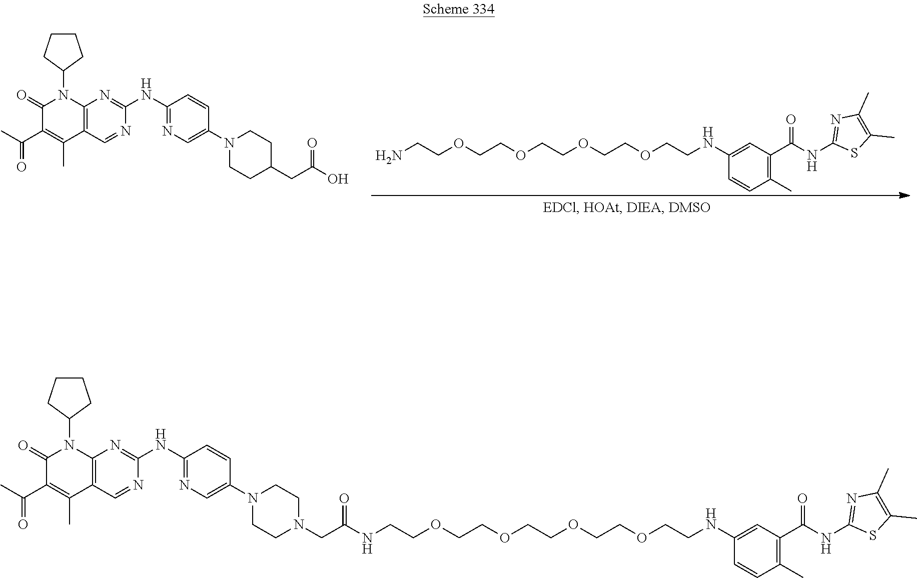 [SMILES: CC(=O)c1c(C)c2cnc(Nc3ccc(N4CCC(CC(=O)O)CC4)cn3)nc2n(C2CCCC2)c1=O ; Cc1ccc(NCCOCCOCCOCCOCCN)cc1C(=O)Nc1nc(C)c(C)s1 ; CC(=O)c1c(C)c2cnc(Nc3ccc(N4CCN(CC(=O)NCCOCCOCCOCCOCCNc5ccc(C)c(C(=O)Nc6nc(C)c(C)s6)c5)CC4)cn3)nc2n(C2CCCC2)c1=O]

CPD-118 was synthesized following the standard procedure for preparing CPD-167 (2.13 mg, 11% yield). MS (ESI) m/z=968.9 [M+H]⁺.

### Example 335. 5-((15-(1-(6-((6-Acetyl-8-cyclopentyl-5-methyl-7-oxo-7,8-dihydropyrido[2,3-d]pyrimidin-2-yl)amino)pyridin-3-yl)piperidin-4-yl)-15-oxo-3,6,9,12-tetraoxapentadecyl)amino)-N-(4,5-dimethylthiazol-2-yl)-2-methylbenzamide (CPD-119)

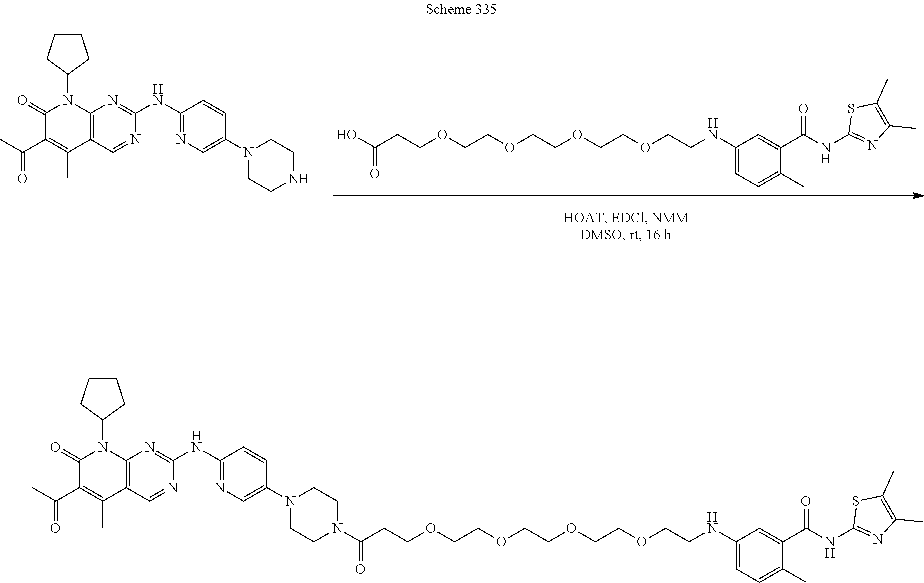

CPD-119 was synthesized following the standard procedure for preparing CPD-042 (TFA salt, 3.0 mg, 2% yield) as a yellow solid. MS (ESI) m/z=940.0 [M+H]⁺.

### Example 336. 3-((2-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-3-oxopropoxy)ethyl)amino)-N-(4,5-dimethylthiazol-2-yl)-2-methylbenzamide (CPD-120)

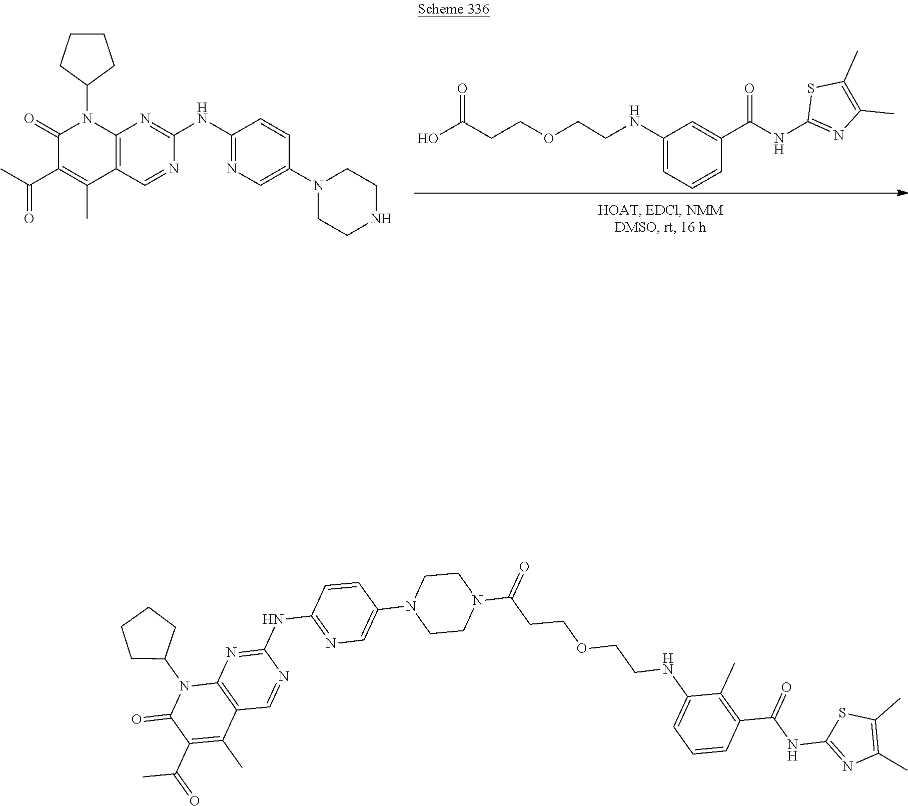 [SMILES: CC(=O)c1c(C)c2cnc(Nc3ccc(N4CCNCC4)cn3)nc2n(C2CCCC2)c1=O ; Cc1nc(NC(=O)c2cccc(NCCOCCC(=O)O)c2)sc1C ; CC(=O)c1c(C)c2cnc(Nc3ccc(N4CCN(C(=O)CCOCCNc5cccc(C(=O)Nc6nc(C)c(C)s6)c5C)CC4)cn3)nc2n(C2CCCC2)c1=O]

CPD-120 was synthesized following the standard procedure for preparing CPD-042 (TFA salt, 5.18 mg, 4% yield) as a yellow solid. MS (ESI) m/z=807.8 [M+H]⁺.

### Example 337. 3-((2-(2-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-3-oxopropoxy)ethoxy)ethyl)amino)-N-(4,5-dimethylthiazol-2-yl)-2-methylbenzamide (CPD-121)

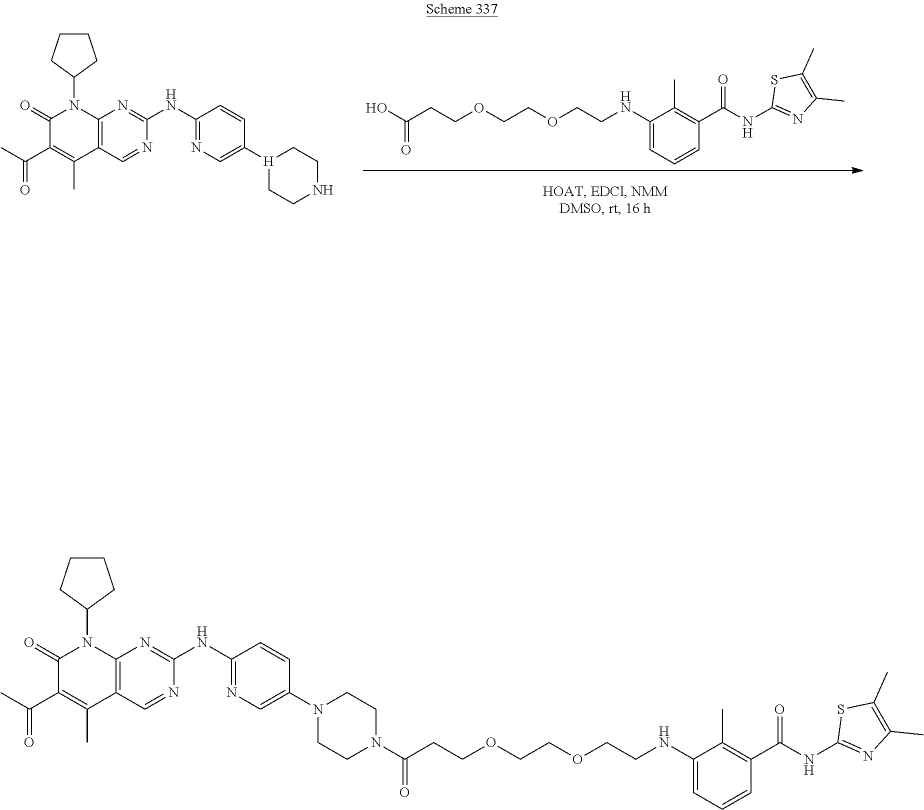

CPD-121 was synthesized following the standard procedure for preparing CPD-042 (TFA salt, 2.53 mg, 2% yield) as a yellow solid. MS (ESI) m/z=851.8 [M+H]⁺.

### Example 338. 3-((2-(2-(2-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-3-oxopropoxy)ethoxy)ethoxy)ethyl)amino)-N-(4,5-dimethylthiazol-2-yl)-2-methylbenzamide (CPD-122)

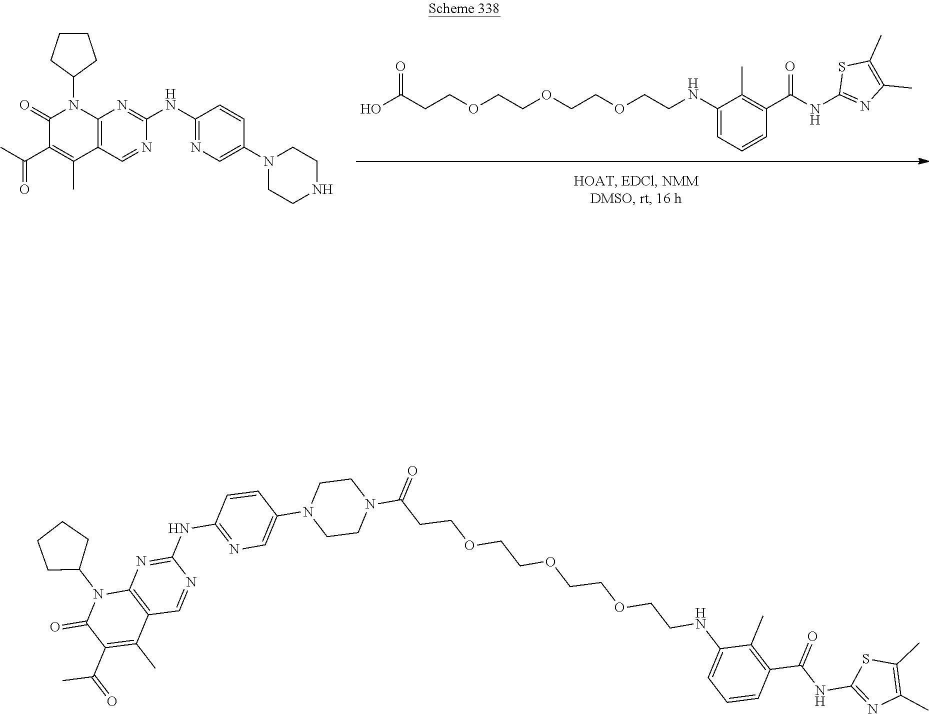 [SMILES: CC(=O)c1c(C)c2cnc(Nc3ccc(N4CCNCC4)cn3)nc2n(C2CCCC2)c1=O ; Cc1nc(NC(=O)c2cccc(NCCOCCOCCOCCC(=O)O)c2C)sc1C ; CC(=O)c1c(C)c2cnc(Nc3ccc(N4CCN(C(=O)CCOCCOCCOCCNc5cccc(C(=O)Nc6nc(C)c(C)s6)c5C)CC4)cn3)nc2n(C2CCCC2)c1=O]

CPD-122 was synthesized following the standard procedure for preparing CPD-042 (TFA salt, 5.26 mg, 4% yield) as a yellow solid. MS (ESI) m/z=895.9 [M+H]⁺.

### Example 339. 3-((15-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-15-oxo-3,6,9,12-tetraoxapentadecyl)amino)-N-(4,5-dimethylthiazol-2-yl)-2-methylbenzamide (CPD-123)

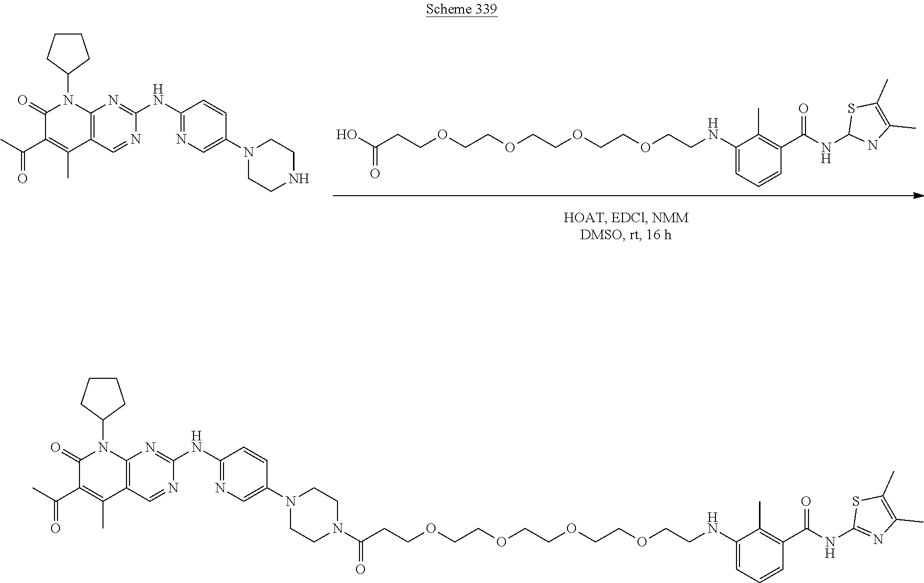

CPD-123 was synthesized following the standard procedure for preparing CPD-042 (TFA salt, 2.22 mg, 2% yield) as a yellow solid. MS (ESI) m/z=940.0 [M+H]⁺.

### Example 340. 3-((2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxoethyl)amino)-N-(4,5-dimethylthiazol-2-yl)-2-methylbenzamide (CPD-124)

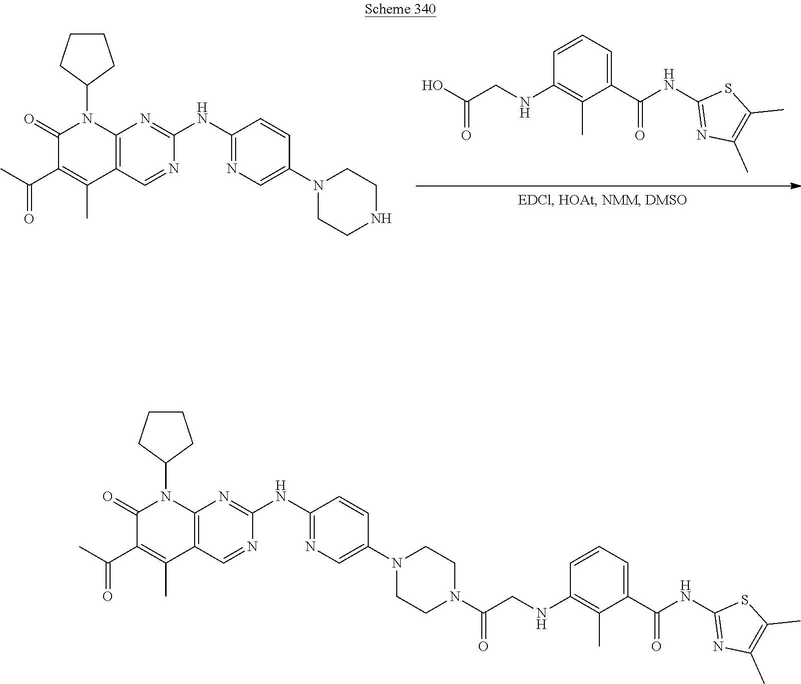 [SMILES: CC(=O)c1c(C)c2cnc(Nc3ccc(N4CCNCC4)cn3)nc2n(C2CCCC2)c1=O ; Cc1nc(NC(=O)c2cccc(NCC(=O)O)c2C)sc1C ; CC(=O)c1c(C)c2cnc(Nc3ccc(N4CCN(C(=O)CNc5cccc(C(=O)Nc6nc(C)c(C)s6)c5C)CC4)cn3)nc2n(C2CCCC2)c1=O]

CPD-124 was synthesized following the standard procedure for preparing CPD-042 (8.3 mg, 61% yield). MS (ESI) m/z=749.8 [M+H]⁺.

### Example 341. 3-((8-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-8-oxooctyl)amino)-N-(4,5-dimethylthiazol-2-yl)-2-methylbenzamide (CPD-125)

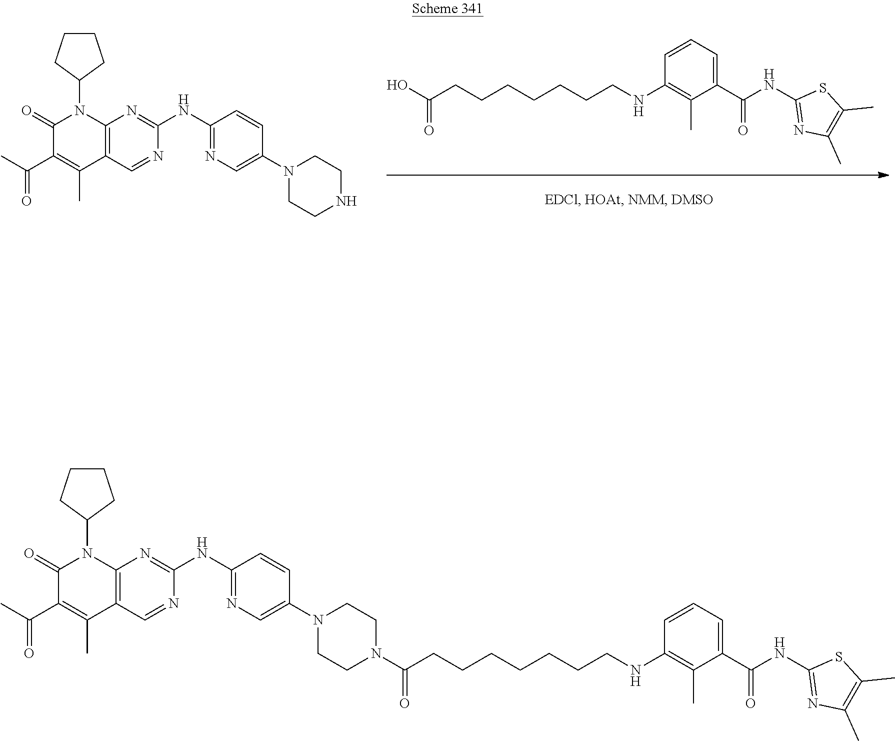 [SMILES: CC(=O)c1c(C)c2cnc(Nc3ccc(N4CCNCC4)cn3)nc2n(C2CCCC2)c1=O ; Cc1nc(NC(=O)c2cccc(NCCCCCCCC(=O)O)c2C)sc1C ; CC(=O)c1c(C)c2cnc(Nc3ccc(N4CCN(C(=O)CCCCCCCNc5cccc(C(=O)Nc6nc(C)c(C)s6)c5C)CC4)cn3)nc2n(C2CCCC2)c1=O]

CPD-125 was synthesized following the standard procedure for preparing CPD-042 (10.2 mg, 68% yield). MS (ESI) m/z=833.8 [M+H]⁺.

### Example 342. 3-((18-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-18-oxo-3,6,9,12,15-pentaoxaoctadecyl)amino)-N-(4,5-dimethylthiazol-2-yl)-2-methylbenzamide (CPD-126)

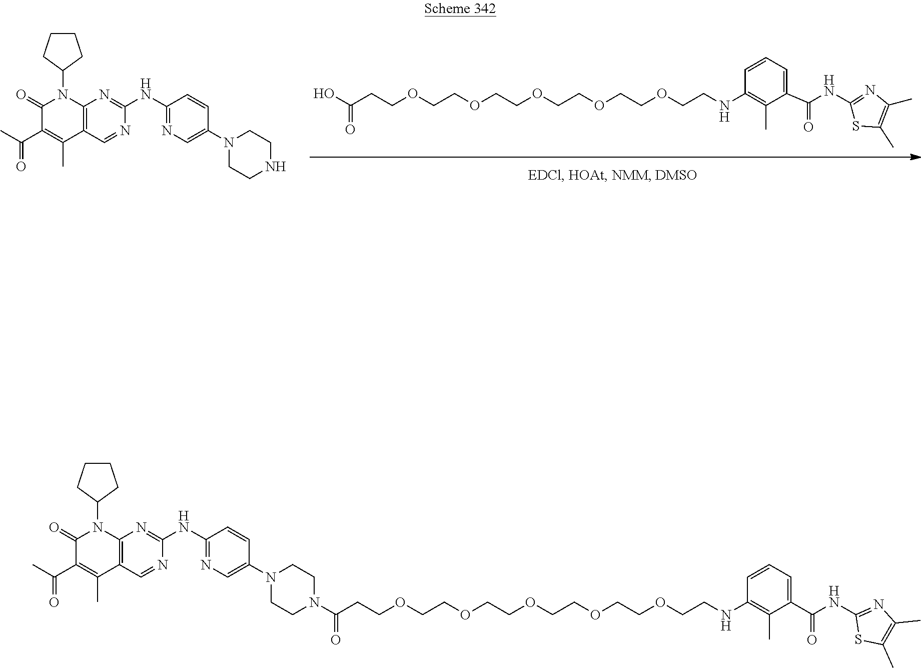

CPD-126 was synthesized following the standard procedure for preparing CPD-042 (10.5 mg, 59% yield). MS (ESI) m/z=984.1 [M+H]⁺.

### Example 343. 5-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12-trioxa-3-azatetradecan-14-yl)amino)-N-(4,5-dimethylthiazol-2-yl)-2-methylbenzamide (CPD-127)

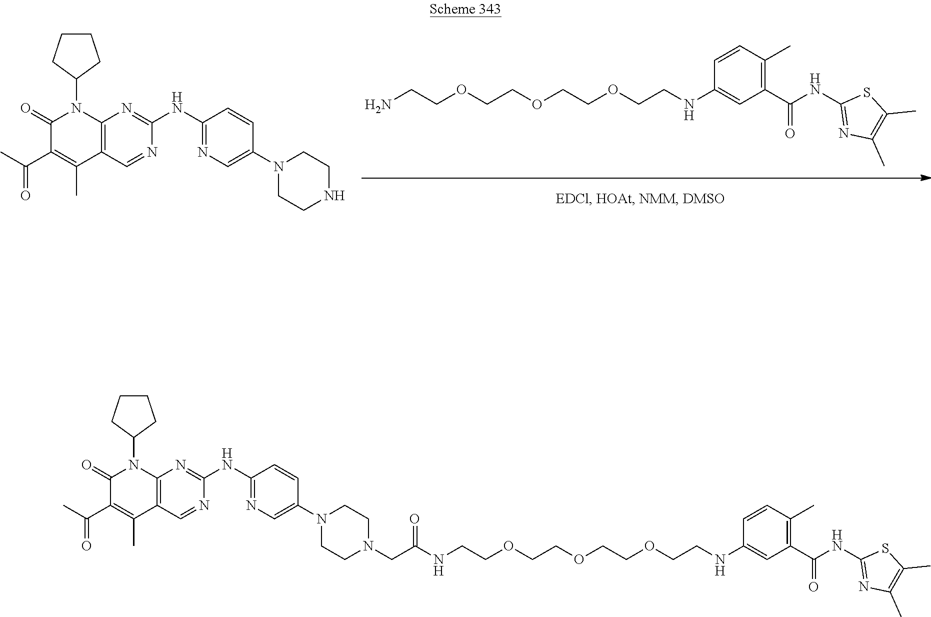 [SMILES: CC(=O)c1c(C)c2cnc(Nc3ccc(N4CCNCC4)cn3)nc2n(C2CCCC2)c1=O ; Cc1ccc(NCCOCCOCCOCCN)cc1C(=O)Nc1nc(C)c(C)s1 ; CC(=O)c1c(C)c2cnc(Nc3ccc(N4CCN(CC(=O)NCCOCCOCCOCCNc5ccc(C)c(C(=O)Nc6nc(C)c(C)s6)c5)CC4)cn3)nc2n(C2CCCC2)c1=O]

CPD-127 was synthesized following the standard procedure for preparing CPD-167 (2.5 mg, 14% yield). MS (ESI) m/z=924.9 [M+H]⁺.

### Example 344. 2-(3-(2-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-3-oxopropoxy)ethoxy)propanamido)-N-(4,5-dimethylthiazol-2-yl)benzamide (CPD-128)

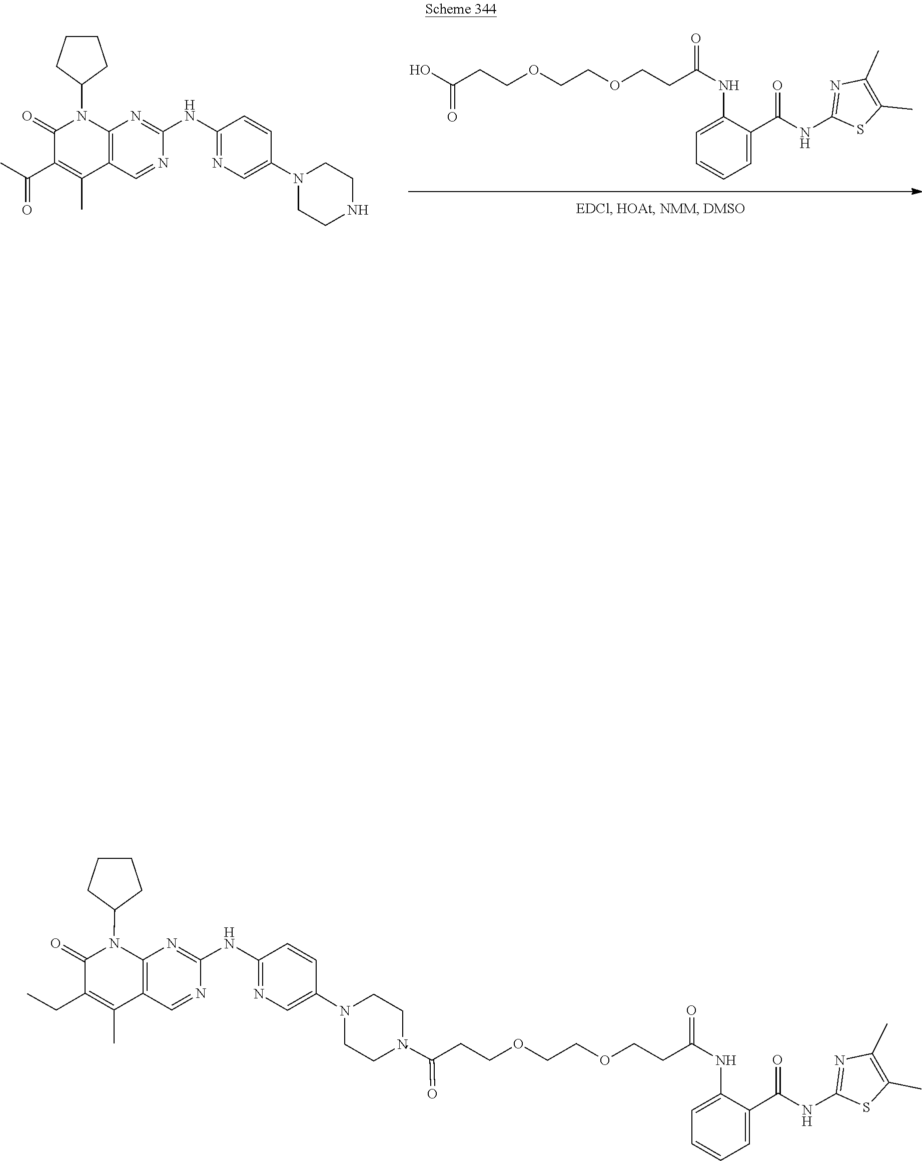

CPD-128 was synthesized following the standard procedure for preparing CPD-042 (7.0 mg, 41% yield). MS (ESI) m/z=865.8 [M+H]⁺.

### Example 345. 5-((6-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-6-oxohexyl)amino)-N-(4,5-dimethylthiazol-2-yl)-2-methylbenzamide (CPD-129)

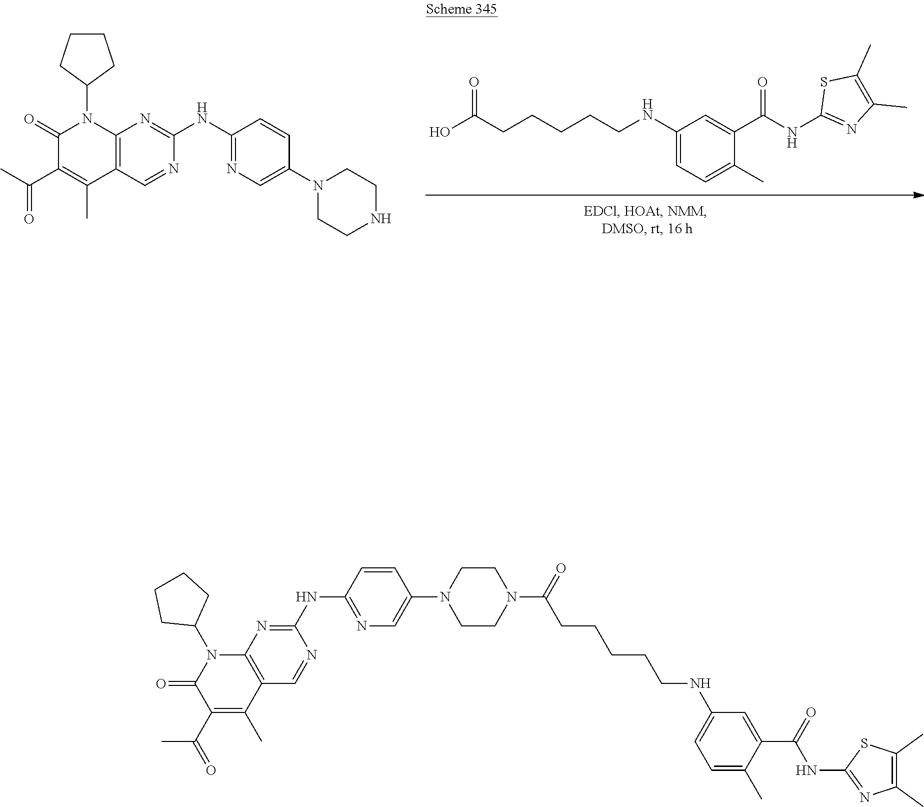 [SMILES: CC(=O)c1c(C)c2cnc(Nc3ccc(N4CCNCC4)cn3)nc2n(C2CCCC2)c1=O ; Cc1ccc(NCCCCCC(=O)O)cc1C(=O)Nc1nc(C)c(C)s1 ; CC(=O)c1c(C)c2cnc(Nc3ccc(N4CCN(C(=O)CCCCCNc5ccc(C)c(C(=O)Nc6nc(C)c(C)s6)c5)CC4)cn3)nc2n(C2CCCC2)c1=O]

CPD-129 was synthesized following the standard procedure for preparing CPD-042 (6.5 mg, 30% yield) as a yellow solid. MS (ESI) m/z=403.65 [M+H]⁺.

### Example 346. 5-((2-(2-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-3-oxopropoxy)ethoxy)ethyl)amino)-N-(4,5-dimethylthiazol-2-yl)-2-methylbenzamide (CPD-130)

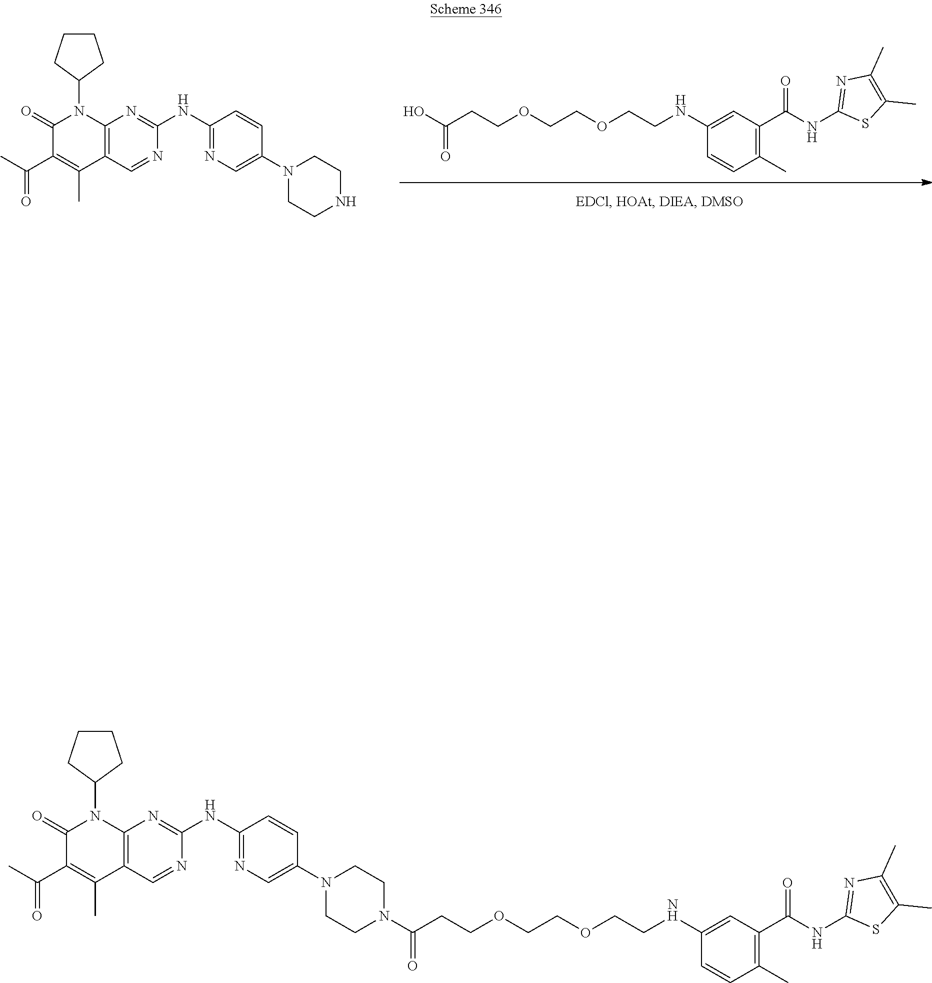 [SMILES: CC(=O)c1c(C)c2cnc(Nc3ccc(N4CCNCC4)cn3)nc2n(C2CCCC2)c1=O ; Cc1ccc(NCCOCCOCCC(=O)O)cc1C(=O)Nc1nc(C)c(C)s1 ; CC(=O)c1c(C)c2cnc(Nc3ccc(N4CCN(C(=O)CCOCCOCCNc5ccc(C)c(C(=O)Nc6nc(C)c(C)s6)c5)CC4)cn3)nc2n(C2CCCC2)c1=O]

CPD-130 was synthesized following the standard procedure for preparing CPD-167 (3.4 mg, 19% yield). MS (ESI) m/z=851.9 [M+H]⁺.

### Example 347. 5-((21-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-21-oxo-3,6,9,12,15,18-hexaoxahenicosyl)amino)-N-(4,5-dimethylthiazol-2-yl)-2-methylbenzamide (CPD-131)

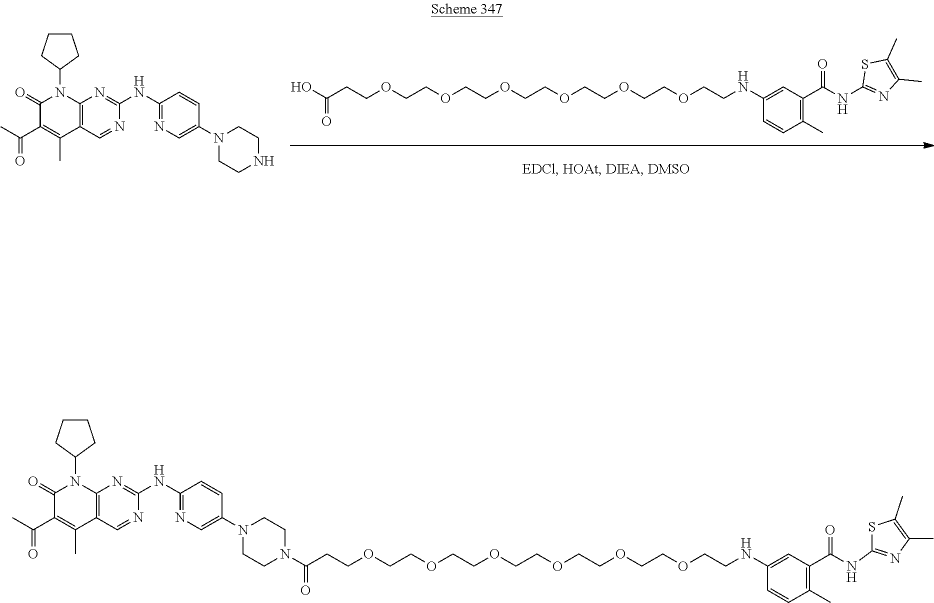

CPD-131 was synthesized following the standard procedure for preparing CPD-001 (2.5 mg, 13% yield). MS (ESI) m/z=1027.9 [M+H]⁺.

### Example 348. 5-((1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18,21-hexaoxa-3-azatricosan-23-yl)amino)-N-(4,5-dimethylthiazol-2-yl)-2-methylbenzamide (CPD-132)

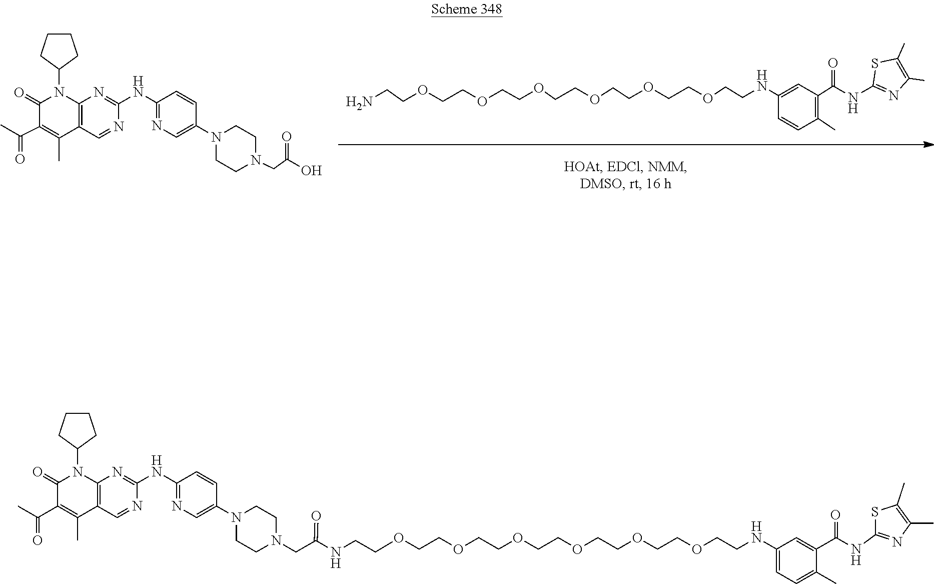 [SMILES: CC(=O)c1c(C)c2cnc(Nc3ccc(N4CCN(CC(=O)O)CC4)cn3)nc2n(C2CCCC2)c1=O ; Cc1ccc(NCCOCCOCCOCCOCCOCCOCCN)cc1C(=O)Nc1nc(C)c(C)s1 ; CC(=O)c1c(C)c2cnc(Nc3ccc(N4CCN(CC(=O)NCCOCCOCCOCCOCCOCCOCCNc5ccc(C)c(C(=O)Nc6nc(C)c(C)s6)c5)CC4)cn3)nc2n(C2CCCC2)c1=O]

CPD-132 was synthesized following the same procedure as CPD-042 (TFA salt, 4.73 mg, 2% yield) as a yellow solid. MS (ESI) m/z=529.08 [M/2+H]⁺.

### Example 349. 3-((21-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-21-oxo-3,6,9,12,15,18-hexaoxahenicosyl)amino)-N-(4,5-dimethylthiazol-2-yl)-2-methylbenzamide (CPD-133)

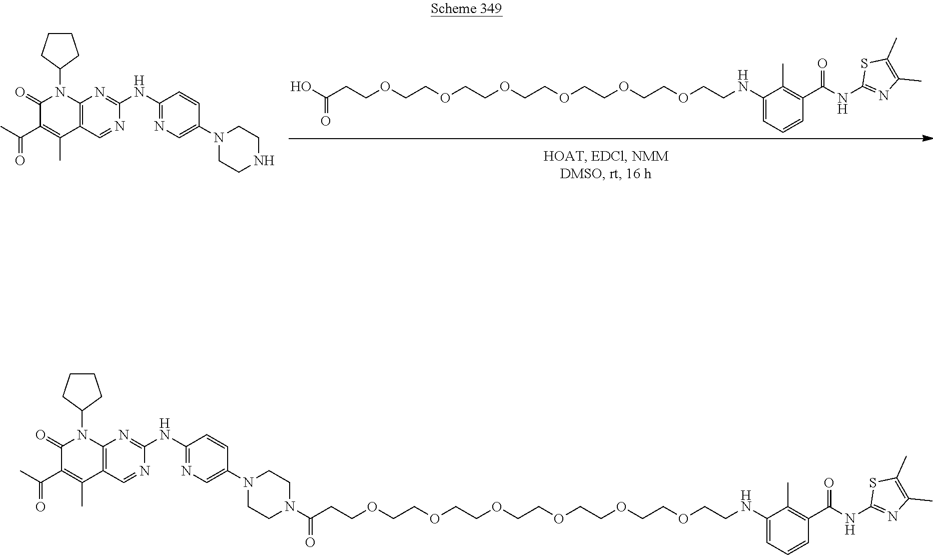

CPD-133 was synthesized following the standard procedure for preparing CPD-042 (8.95 mg, 52% yield) as a yellow solid. MS (ESI) m/z=1028.1 [M+H]⁺.

### Example 350. 5-((18-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-18-oxo-3,6,9,12,15-pentaoxaoctadecyl)amino)-N-(4,5-dimethylthiazol-2-yl)-2-methylbenzamide (CPD-134)

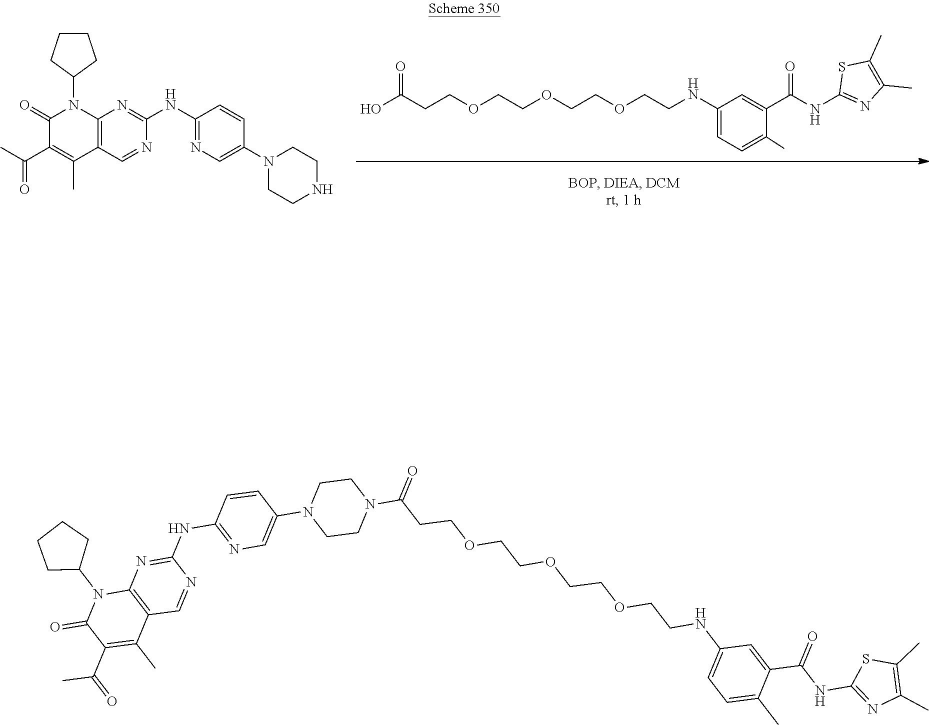 [SMILES: CC(=O)c1c(C)c2cnc(Nc3ccc(N4CCNCC4)cn3)nc2n(C2CCCC2)c1=O ; Cc1ccc(NCCOCCOCCOCCC(=O)O)cc1C(=O)Nc1nc(C)c(C)s1 ; CC(=O)c1c(C)c2cnc(Nc3ccc(N4CCN(C(=O)CCOCCOCCOCCNc5ccc(C)c(C(=O)Nc6nc(C)c(C)s6)c5)CC4)cn3)nc2n(C2CCCC2)c1=O]

To a solution of 6-acetyl-8-cyclopentyl-5-methyl-2-((5-(piperazin-1-yl)pyridin-2-yl)amino)pyrido[2,3-d]pyrimidin-7(8H)-one (8.89 mg, 0.020 mmol) and 1-((3-((4,5-dimethylthiazol-2-yl)carbamoyl)-4-methylphenyl)amino)-3,6,9,12,15-pentaoxaoctadecan-18-oic acid (10 mg, 0.018 mmol) in DCM (1.0 mL) were added BOP (14.75 mg, 0.072 mmol) and DIPEA (11.67 mg, 0.090 mmol, 14.93 μL). The reaction mixture was stirred at rt for 1 h. Upon completion, the reaction mixture was concentrated under reduced pressure. The residue was purified by reverse-phase chromatography to provide the title compound (TFA salt, 15.82 mg, 13% yield) as a yellow solid. MS (ESI) m/z=983.9 [M+H]⁺.

### Example 351. 5-((2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxoethyl)amino)-N-(4,5-dimethylthiazol-2-yl)-2-methylbenzamide (CPD-135)

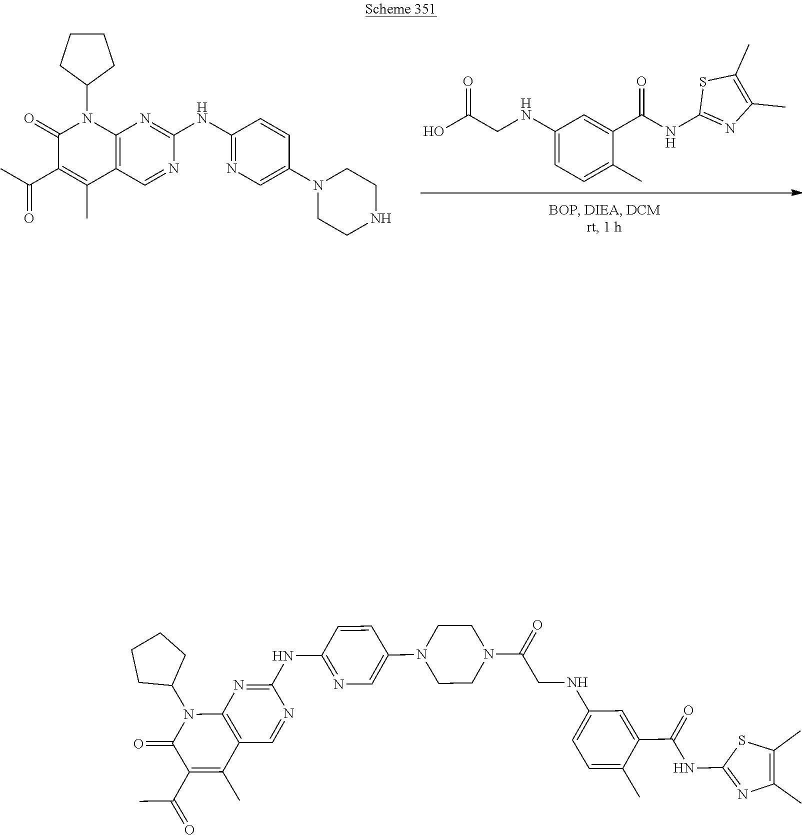 [SMILES: CC(=O)c1c(C)c2cnc(Nc3ccc(N4CCNCC4)cn3)nc2n(C2CCCC2)c1=O ; Cc1ccc(NCC(=O)O)cc1C(=O)Nc1nc(C)c(C)s1 ; CC(=O)c1c(C)c2cnc(Nc3ccc(N4CCN(C(=O)CNc5ccc(C)c(C(=O)Nc6nc(C)c(C)s6)c5)CC4)cn3)nc2n(C2CCCC2)c1=O]

CPD-135 was synthesized following the standard procedure for preparing CPD-134 (TFA salt, 24.06 mg, 15% yield) as a yellow solid. MS (ESI) m/z=749.8 [M+H]⁺.

### Example 352. 2-(8-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)octanamido)-N-(4,5-dimethylthiazol-2-yl)benzamide (CPD-136)

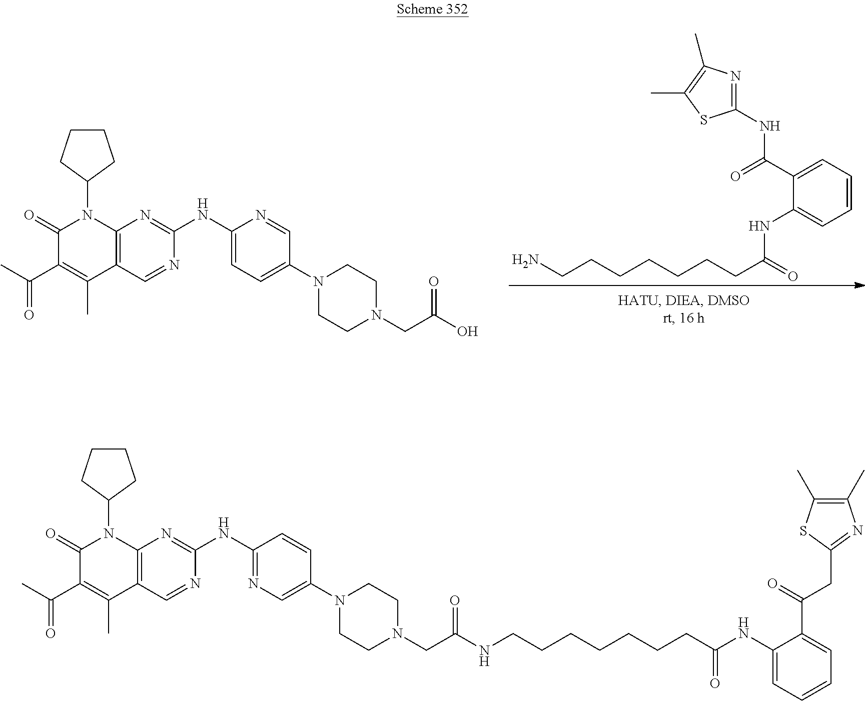 [SMILES: CC(=O)c1c(C)c2cnc(Nc3ccc(N4CCN(CC(=O)O)CC4)cn3)nc2n(C2CCCC2)c1=O ; Cc1nc(NC(=O)c2ccccc2NC(=O)CCCCCCCN)sc1C ; CC(=O)c1c(C)c2cnc(Nc3ccc(N4CCN(CC(=O)NCCCCCCCC(=O)Nc5ccccc5C(=O)Cc5nc(C)c(C)s5)CC4)cn3)nc2n(C2CCCC2)c1=O]

To a solution of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid (11.3 mg, 0.022 mmol) and 2-(8-aminooctanamido)-N-(4,5-dimethylthiazol-2-yl)benzamide (9.55 mg, 0.025 mmol) in DMSO (0.5 mL) were added HATU (12.75 mg, 0.034 mmol) and DIPEA (14.44 mg, 0.112 mmol, 18.47 μL). The reaction mixture was stirred at rt for 16 h. Upon completion, the mixture was purified by reverse-phase chromatography to provide the title compound (TFA salt, 0.94 mg, 1% yield) as a yellow solid. MS (ESI) m/z=439.1 [M/2+H]⁺.

### Example 353. 3-((6-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-6-oxohexyl)amino)-N-(4,5-dimethylthiazol-2-yl)-2-methylbenzamide (CPD-137)

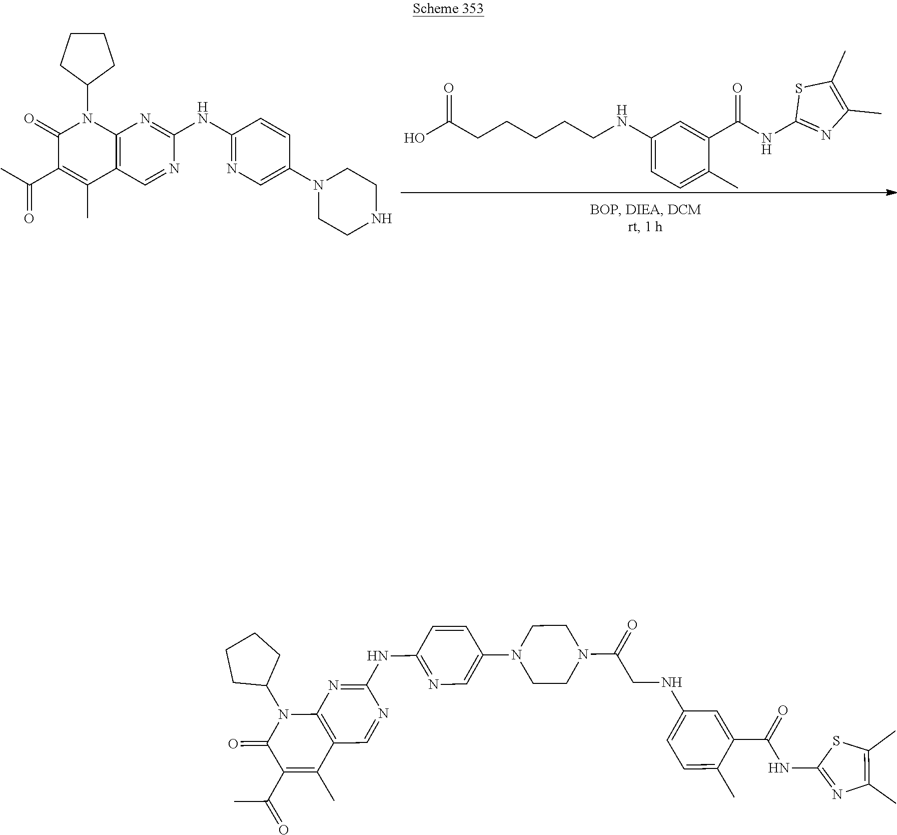 [SMILES: CC(=O)c1c(C)c2cnc(Nc3ccc(N4CCNCC4)cn3)nc2n(C2CCCC2)c1=O ; Cc1ccc(NCCCCCC(=O)O)cc1C(=O)Nc1nc(C)c(C)s1 ; CC(=O)c1c(C)c2cnc(Nc3ccc(N4CCN(C(=O)CNc5ccc(C)c(C(=O)Nc6nc(C)c(C)s6)c5)CC4)cn3)nc2n(C2CCCC2)c1=O]

CPD-137 was synthesized following the standard procedure for preparing CPD-134 (TFA salt, 15.82 mg, 11% yield: 11%) as a yellow solid. MS (ESI) m/z=805.8 [M+H]⁺.

### Example 354. 3-((7-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-7-oxoheptyl)amino)-N-(4,5-dimethylthiazol-2-yl)-2-methylbenzamide (CPD-138)

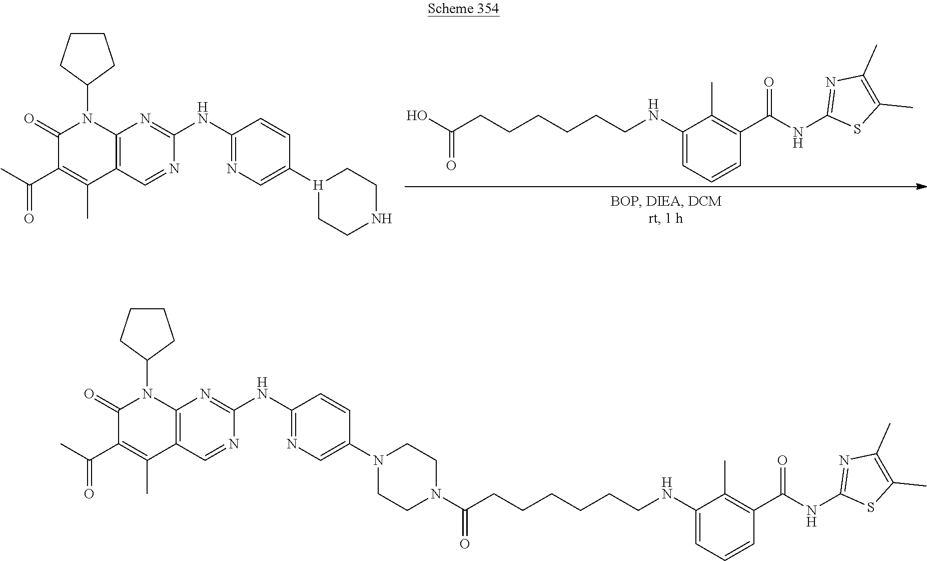

CPD-138 was synthesized following the standard procedure for preparing CPD-134 (TFA salt, 16.08 mg, 11% yield) as yellow solid. MS (ESI) m/z=819.9 [M+H]⁺.

### Example 355. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4,5-dimethylthiazol-2-yl)-6-methylbenzamide (CPD-139)

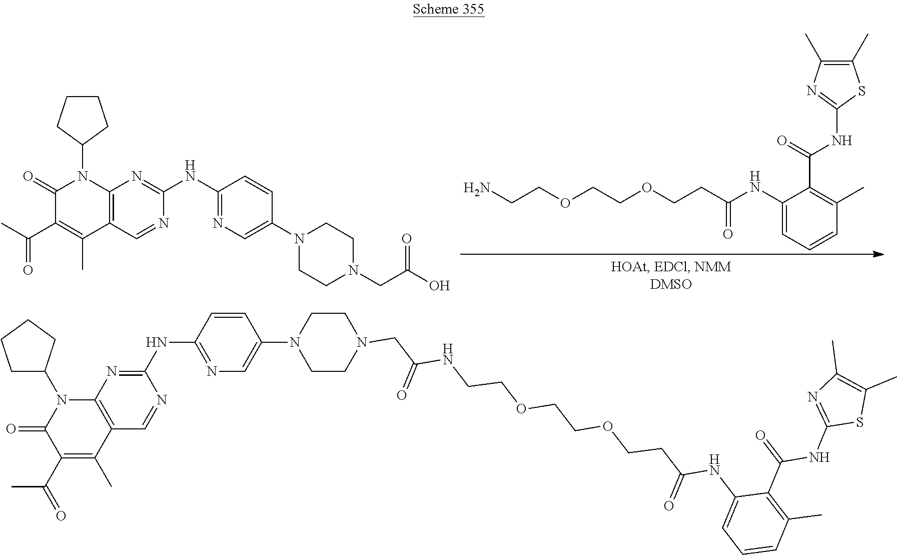

CPD-139 was synthesized following the standard procedure for preparing CPD-042 (TFA salt, 8.0 mg, 40% yield) as a yellow solid. MS (ESI) m/z=909.0 [M+H]⁺.

### Example 356. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-4-chloro-N-(4,5-dimethylthiazol-2-yl)benzamide (CPD-140)

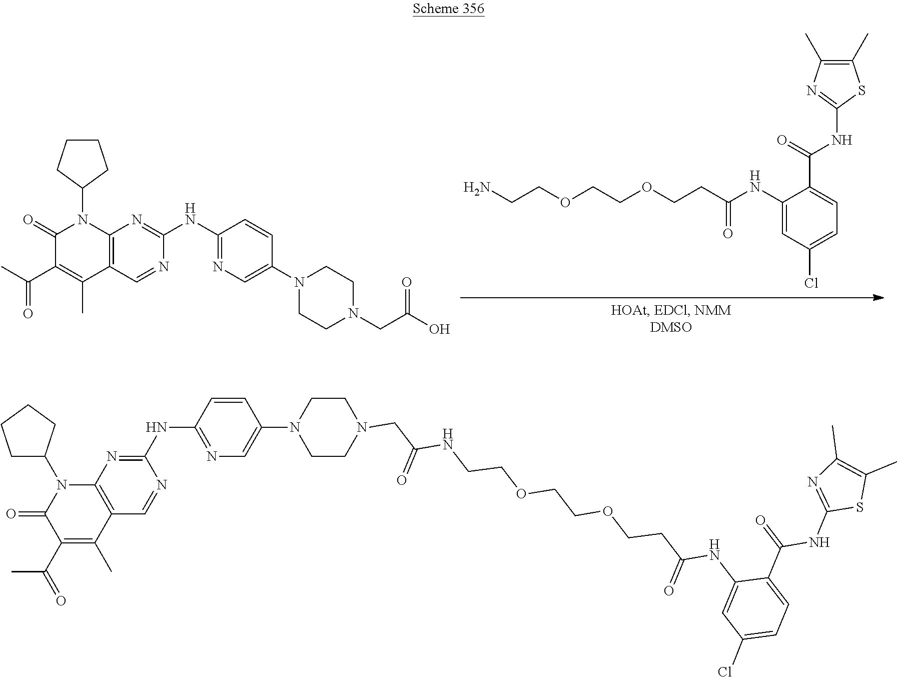 [SMILES: CC(=O)c1c(C)c2cnc(Nc3ccc(N4CCN(CC(=O)O)CC4)cn3)nc2n(C2CCCC2)c1=O ; Cc1nc(NC(=O)c2ccc(Cl)cc2NC(=O)CCOCCOCCN)sc1C ; CC(=O)c1c(C)c2cnc(Nc3ccc(N4CCN(CC(=O)NCCOCCOCCC(=O)Nc5cc(Cl)ccc5C(=O)Nc5nc(C)c(C)s5)CC4)cn3)nc2n(C2CCCC2)c1=O]

CPD-140 was synthesized following the standard procedure for preparing CPD-042 (8.20 mg, 40% yield). MS (ESI) m/z=928.9 [M+H]⁺.

### Example 357. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4,5-dimethylthiazol-2-yl)-5-methylbenzamide (CPD-141)

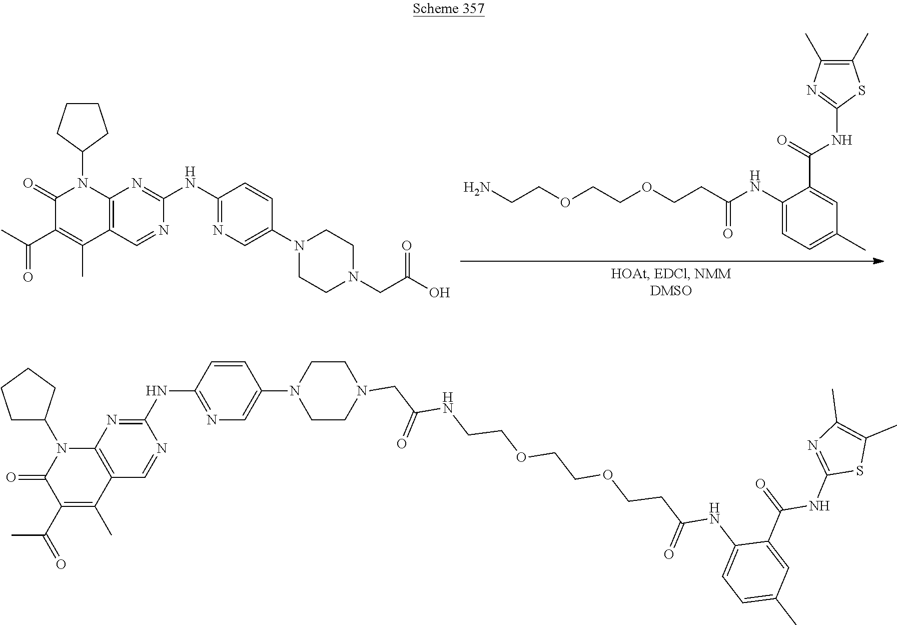 [SMILES: CC(=O)c1c(C)c2cnc(Nc3ccc(N4CCN(CC(=O)O)CC4)cn3)nc2n(C2CCCC2)c1=O ; Cc1ccc(NC(=O)CCOCCOCCN)c(C(=O)Nc2nc(C)c(C)s2)c1 ; CC(=O)c1c(C)c2cnc(Nc3ccc(N4CCN(CC(=O)NCCOCCOCCC(=O)Nc5ccc(C)cc5C(=O)Nc5nc(C)c(C)s5)CC4)cn3)nc2n(C2CCCC2)c1=O]

CPD-141 was synthesized following the standard procedure for preparing CPD-042 (7.10 mg, 35% yield). MS (ESI) m/z=909.0 [M+H]⁺.

### Example 358. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-5-chloro-N-(4,5-dimethylthiazol-2-yl)benzamide (CPD-142)

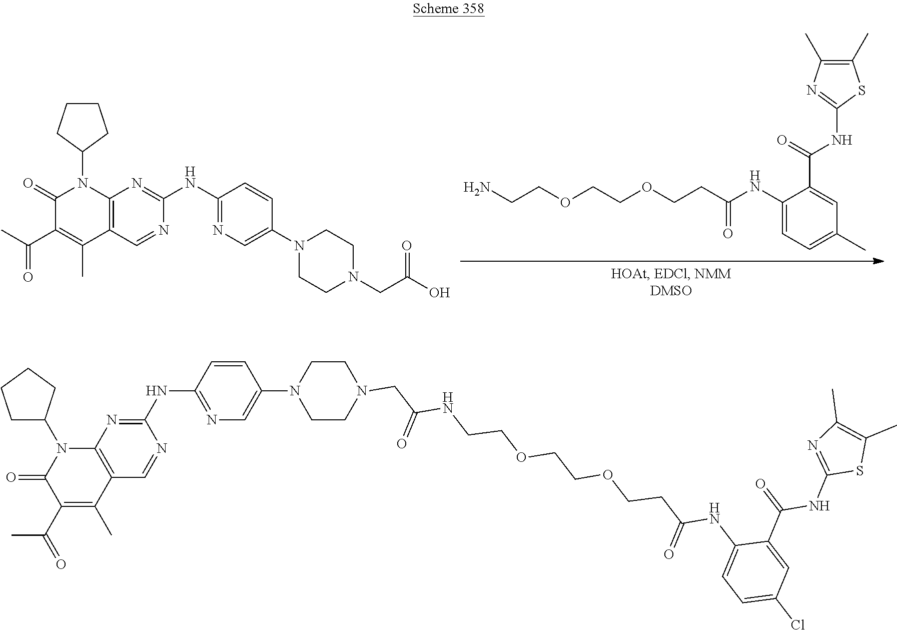 [SMILES: CC(=O)c1c(C)c2cnc(Nc3ccc(N4CCN(CC(=O)O)CC4)cn3)nc2n(C2CCCC2)c1=O ; Cc1ccc(NC(=O)CCOCCOCCN)c(C(=O)Nc2nc(C)c(C)s2)c1 ; CC(=O)c1c(C)c2cnc(Nc3ccc(N4CCN(CC(=O)NCCOCCOCCC(=O)Nc5ccc(Cl)cc5C(=O)Nc5nc(C)c(C)s5)CC4)cn3)nc2n(C2CCCC2)c1=O]

CPD-142 was synthesized following the standard procedure for preparing CPD-042 (3.06 mg, 30% yield) MS (ESI) m/z=910.0 [M+H]⁺.

### Example 359. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4,5-dimethylthiazol-2-yl)-4-fluorobenzamide (CPD-143)

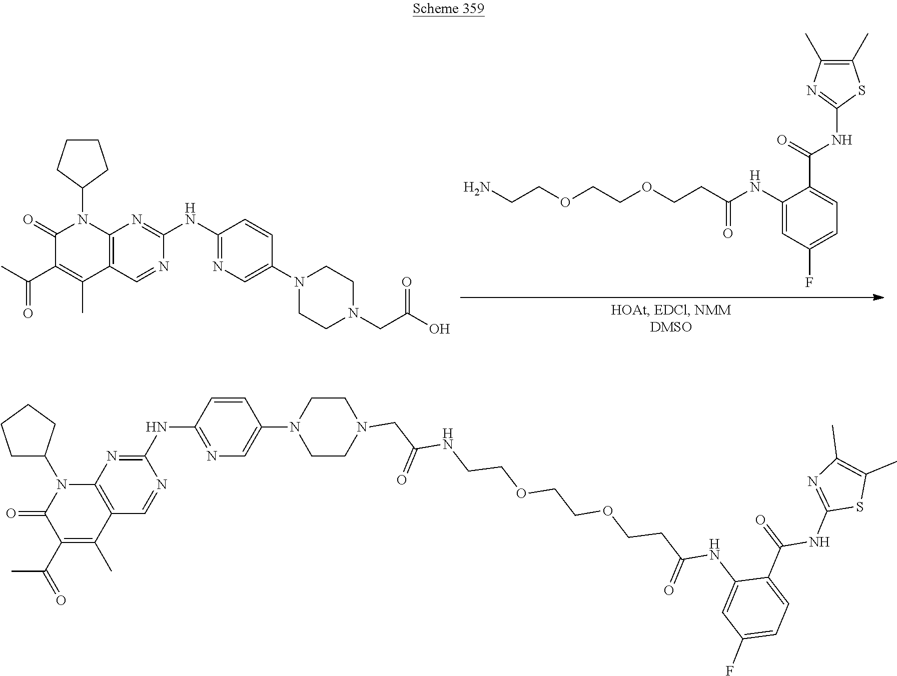 [SMILES: CC(=O)c1c(C)c2cnc(Nc3ccc(N4CCN(CC(=O)O)CC4)cn3)nc2n(C2CCCC2)c1=O ; Cc1nc(NC(=O)c2ccc(F)cc2NC(=O)CCOCCOCCN)sc1C ; CC(=O)c1c(C)c2cnc(Nc3ccc(N4CCN(CC(=O)NCCOCCOCCC(=O)Nc5cc(F)ccc5C(=O)Nc5nc(C)c(C)s5)CC4)cn3)nc2n(C2CCCC2)c1=O]

CPD-143 was synthesized following the standard procedure for preparing CPD-042 (1.44 mg, 14% yield). MS (ESI) m/z=912.8 [M+H]⁺.

### Example 360. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-4-bromo-N-(4,5-dimethylthiazol-2-yl)benzamide (CPD-144)

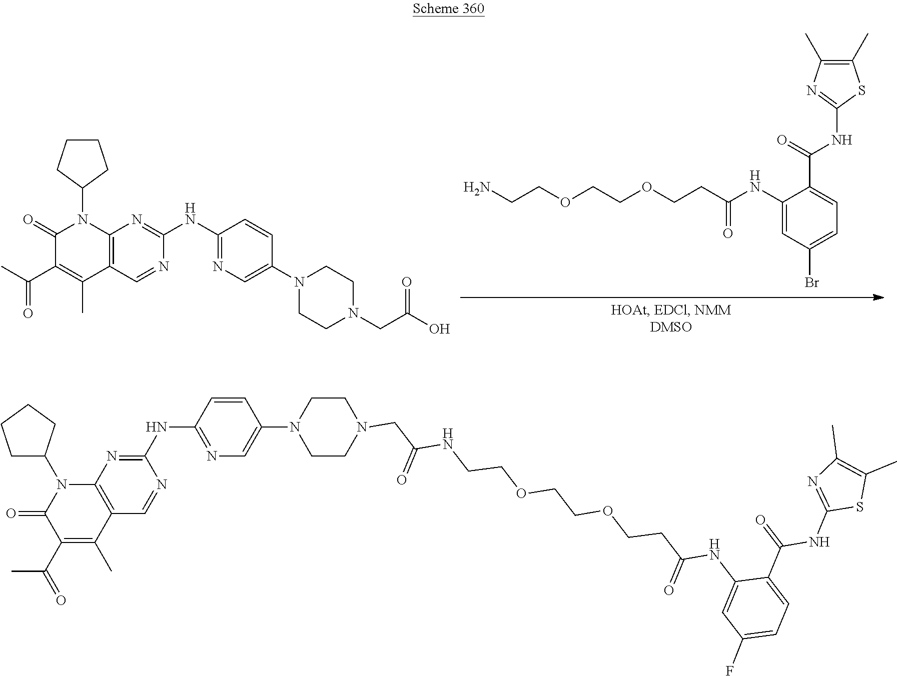 [SMILES: CC(=O)c1c(C)c2cnc(Nc3ccc(N4CCN(CC(=O)O)CC4)cn3)nc2n(C2CCCC2)c1=O ; Cc1nc(NC(=O)c2ccc(Br)cc2NC(=O)CCOCCOCCN)sc1C ; CC(=O)c1c(C)c2cnc(Nc3ccc(N4CCN(CC(=O)NCCOCCOCCC(=O)Nc5cc(F)ccc5C(=O)Nc5nc(C)c(C)s5)CC4)cn3)nc2n(C2CCCC2)c1=O]

CPD-144 was synthesized following the standard procedure for preparing CPD-042 (1.98 mg, 18% yield). MS (ESI) m/z=974.7 [M+H]⁺.

### Example 361. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-5-bromo-N-(4,5-dimethylthiazol-2-yl)benzamide (CPD-145)

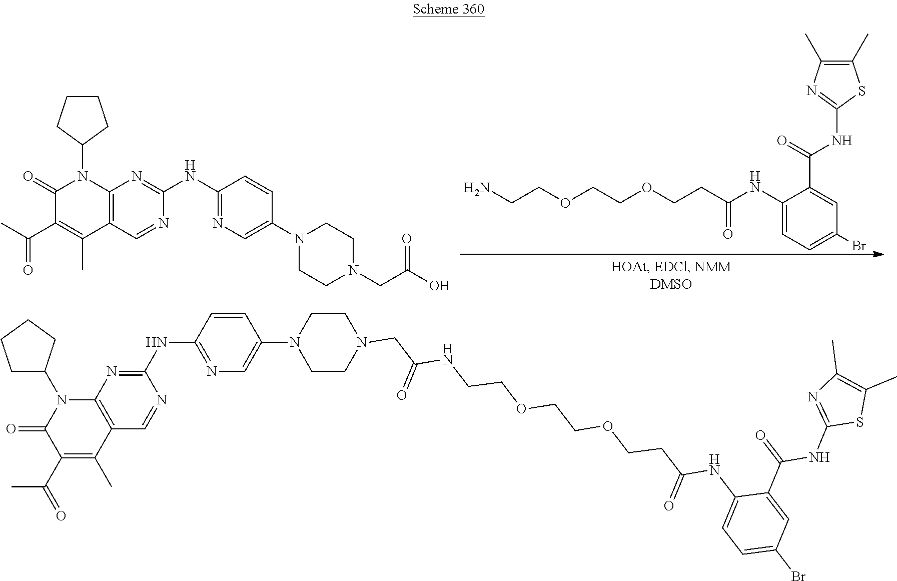

CPD-145 was synthesized following the standard procedure for preparing CPD-042 (1.45 mg, 13% yield). MS (ESI) m/z=974.7 [M+H]⁺.

### Example 362. 2-(3-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)propanamido)-N-(4,5-dimethylthiazol-2-yl)benzamide (CPD-146)

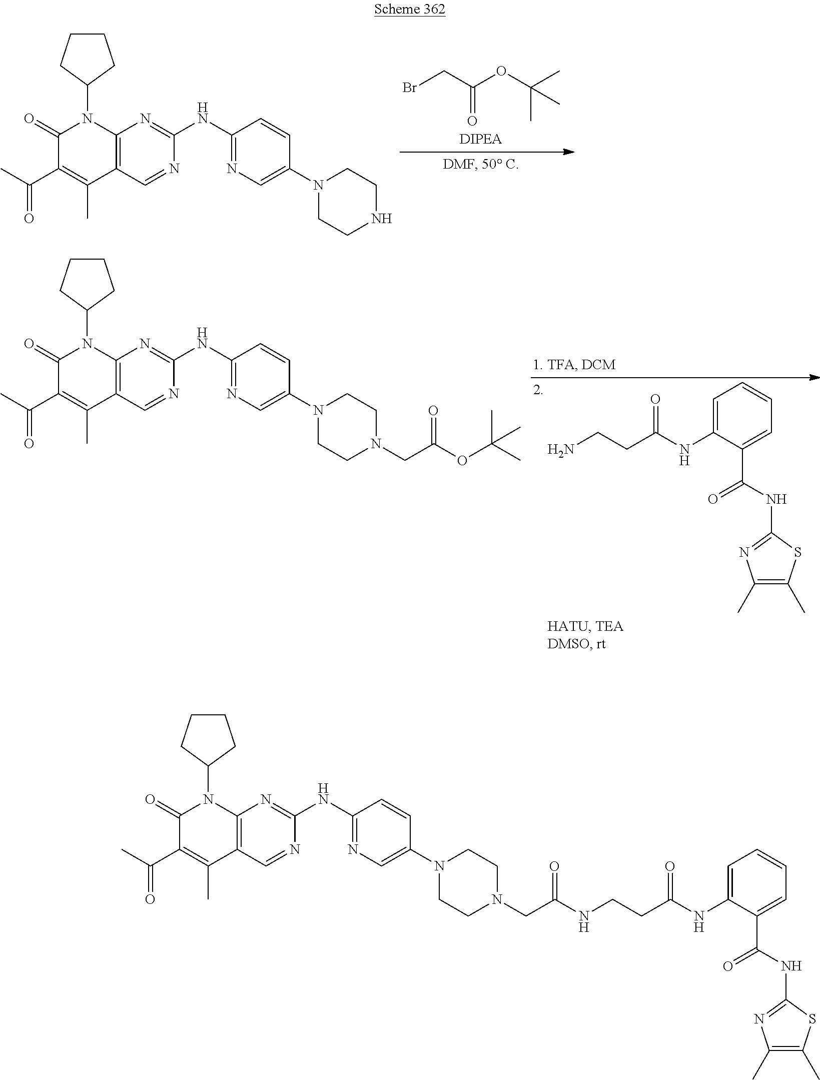

### Step 1. Synthesis of tert-butyl 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetate

To a solution of 6-acetyl-8-cyclopentyl-5-methyl-2-((5-(piperazin-1-yl)pyridin-2-yl)amino)pyrido[2,3-d]pyrimidin-7(8H)-one (100 mg, 223.4 μmol) in DMF (5 mL) were added tert-butyl 2-bromoacetate (87 mg, 446.9 μmol) and DIPEA (57 mg, 446.9 μmol). The mixture was stirred at rt for 4 h, and then purified by reverse-phase chromatography (0.1% TFA in water:MeOH=1:1) to provide the title compound (105 mg, 84% yield) as a yellow solid. MS (ESI) m/z=562.4 [M+H]⁺.

### Step 2. Synthesis of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid

To a solution of tert-butyl 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetate (105 mg, 186.9 μmol) in DCM (1 mL) was added TFA (1 mL). After the mixture was stirred at rt for 30 min, it was concentrated under reduced pressure. The residue was purified by reverse-phase chromatography (water/MeOH=1:1) to provide the title compound (90 mg, 95% yield) as a yellow solid. MS (ESI) m/z=506.3 [M+H]⁺.

### Step 3. Synthesis of 2-(3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)propanamido)-N-(4,5-dimethylthiazol-2-yl)benzamide

To a mixture of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid (10 mg, 19.9 μmol) and 2-(3-aminopropanoylamino)-N-(4,5-dimethylthiazol-2-yl)benzamide (6 mg, 19.8 μmol) in DMSO (0.5 mL) were added HATU (15 mg, 39 μmol) and TEA (7 mg, 59.3 μmol). The reaction mixture was stirred at rt for 1 h, then purified by reverse phase chromatography (0-70% MeCN in H₂O) to provide the title compound (2.4 mg, 15% yield) as a yellow solid. MS (ESI) m/z=806.8 [M+H]⁺.

### Example 363. 2-(3-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)propanamido)-N-(4,5-dimethylthiazol-2-yl)benzamide (CPD-147)

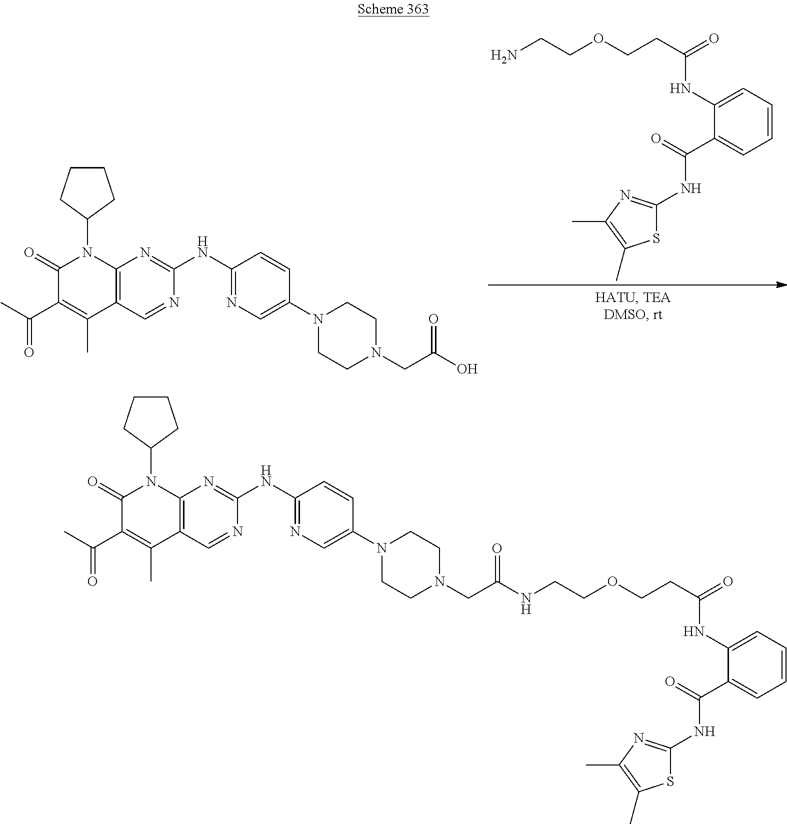 [SMILES: CC(=O)c1c(C)c2cnc(Nc3ccc(N4CCN(CC(=O)O)CC4)cn3)nc2n(C2CCCC2)c1=O ; Cc1nc(NC(=O)c2ccccc2NC(=O)CCOCCN)sc1C ; CC(=O)c1c(C)c2cnc(Nc3ccc(N4CCN(CC(=O)NCCOCCC(=O)Nc5ccccc5C(=O)Nc5nc(C)c(C)s5)CC4)cn3)nc2n(C2CCCC2)c1=O]

CPD-147 was synthesized following the standard procedure for preparing CPD-146 (2.4 mg, 14% yield). MS (ESI) m/z=850.8 [M+H]⁺.

### Example 364. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-5-(butylamino)-N-(4,5-dimethylthiazol-2-yl)benzamide (CPD-148)

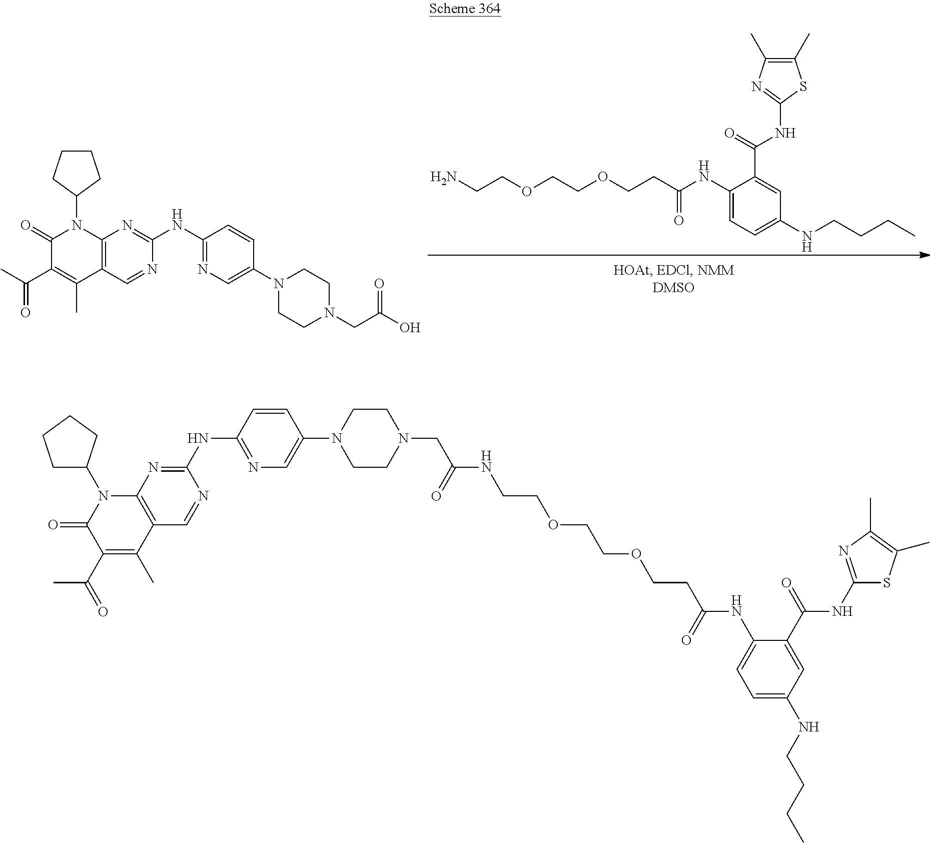 [SMILES: CC(=O)c1c(C)c2cnc(Nc3ccc(N4CCN(CC(=O)O)CC4)cn3)nc2n(C2CCCC2)c1=O ; CCCCNc1ccc(NC(=O)CCOCCOCCN)c(C(=O)Nc2nc(C)c(C)s2)c1 ; CCCCNc1ccc(NC(=O)CCOCCOCCNC(=O)CN2CCN(c3ccc(Nc4ncc5c(C)c(C(C)=O)c(=O)n(C6CCCC6)c5n4)nc3)CC2)c(C(=O)Nc2nc(C)c(C)s2)c1]

CPD-148 was synthesized following the standard procedure for preparing CPD-042 (2.19 mg, 20% yield). MS (ESI) m/z=966.0 [M+H]⁺.

### Example 365. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4,5-dimethylthiazol-2-yl)-4-methylbenzamide (CPD-149)

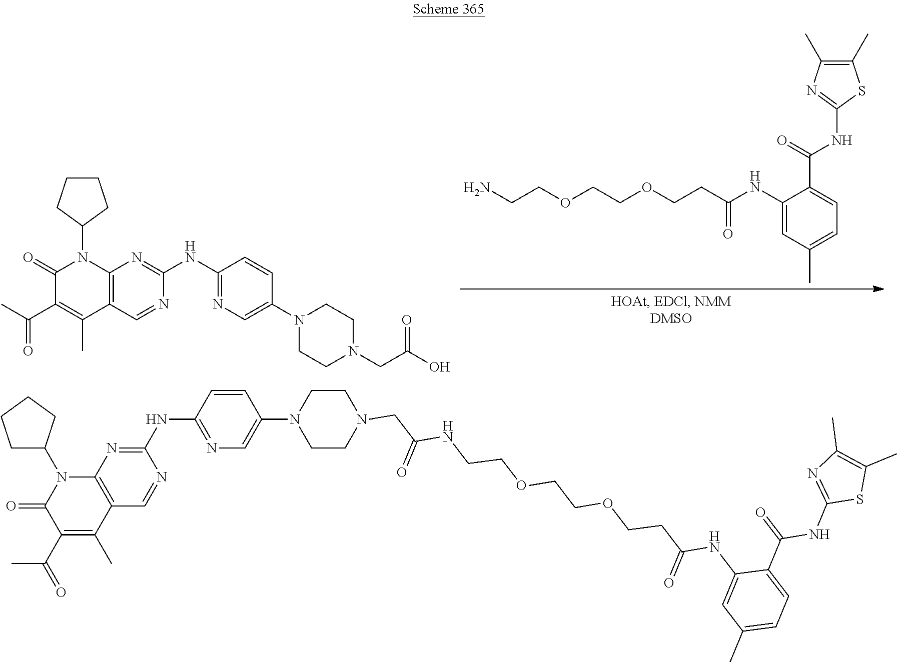

CPD-149 was synthesized following the standard procedure for preparing CPD-042 (1.87 mg, 18% yield). MS (ESI) m/z=908.8 [M+H]⁺.

### Example 366. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4,5-dimethylthiazol-2-yl)-5-(methylamino)benzamide (CPD-150)

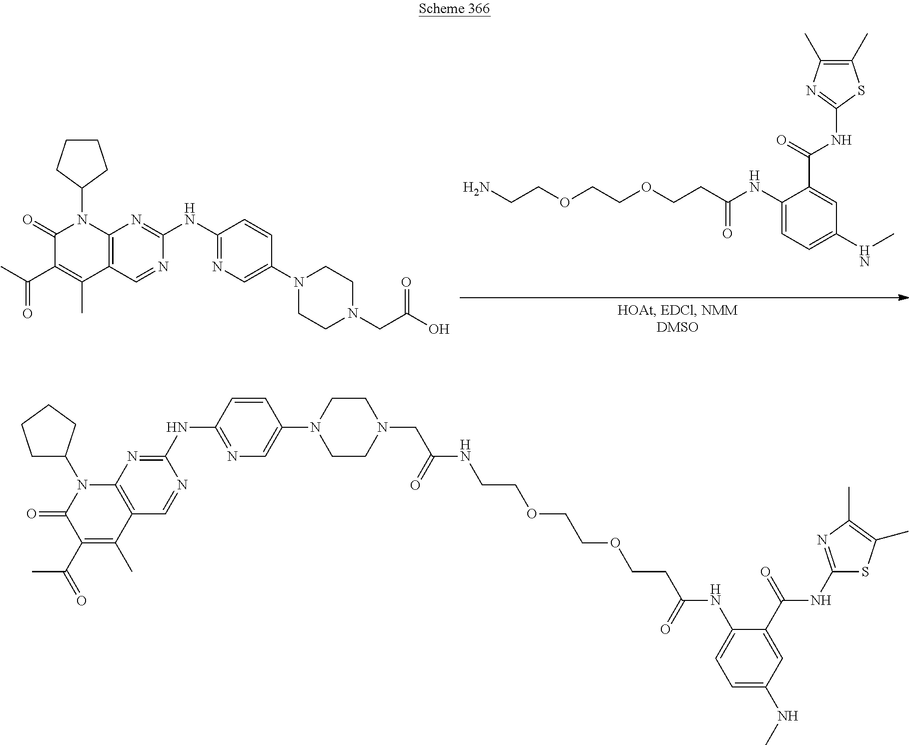 [SMILES: CC(=O)c1c(C)c2cnc(Nc3ccc(N4CCN(CC(=O)O)CC4)cn3)nc2n(C2CCCC2)c1=O ; CNc1ccc(NC(=O)CCOCCOCCN)c(C(=O)Nc2nc(C)c(C)s2)c1 ; CNc1ccc(NC(=O)CCOCCOCCNC(=O)CN2CCN(c3ccc(Nc4ncc5c(C)c(C(C)=O)c(=O)n(C6CCCC6)c5n4)nc3)CC2)c(C(=O)Nc2nc(C)c(C)s2)c1]

CPD-150 was synthesized following the standard procedure for preparing CPD-042 (2.33 mg, 22% yield). MS (ESI) m/z=923.8 [M+H]⁺.

### Example 367. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-5-(dimethylamino)-N-(4,5-dimethylthiazol-2-yl)benzamide (CPD-151)

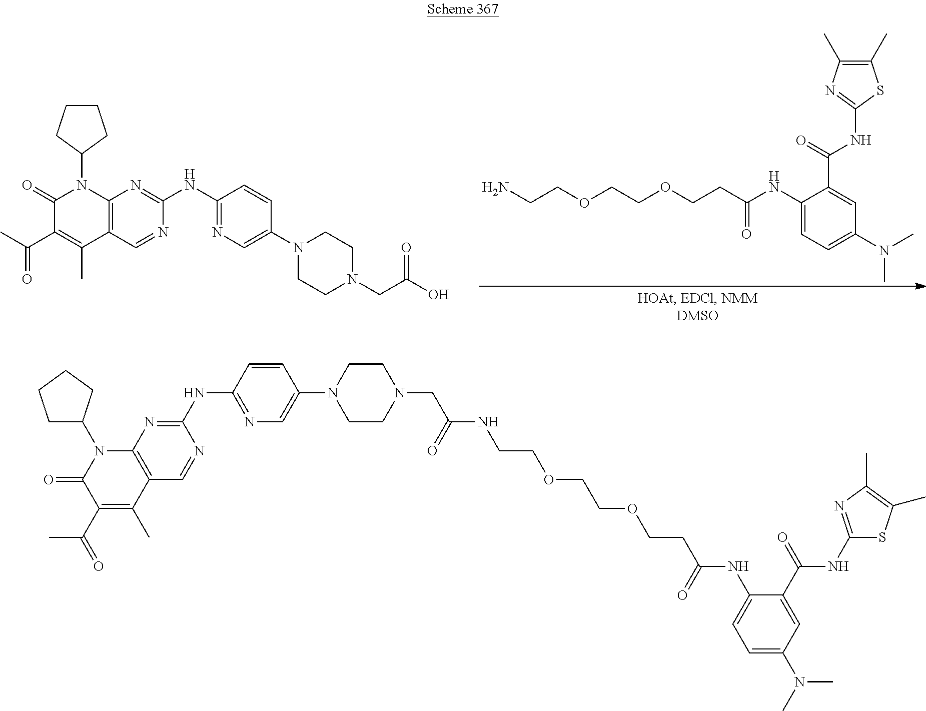 [SMILES: CC(=O)c1c(C)c2cnc(Nc3ccc(N4CCN(CC(=O)O)CC4)cn3)nc2n(C2CCCC2)c1=O ; Cc1nc(NC(=O)c2cc(N(C)C)ccc2NC(=O)CCOCCOCCN)sc1C ; CC(=O)c1c(C)c2cnc(Nc3ccc(N4CCN(CC(=O)NCCOCCOCCC(=O)Nc5ccc(N(C)C)cc5C(=O)Nc5nc(C)c(C)s5)CC4)cn3)nc2n(C2CCCC2)c1=O]

CPD-151 was synthesized following the standard procedure for preparing CPD-042 (2.12 mg, 20% yield). MS (ESI) m/z=937.8 [M+H]⁺.

### Example 368. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4,5-dimethylthiazol-2-yl)-5-fluorobenzamide (CPD-152)

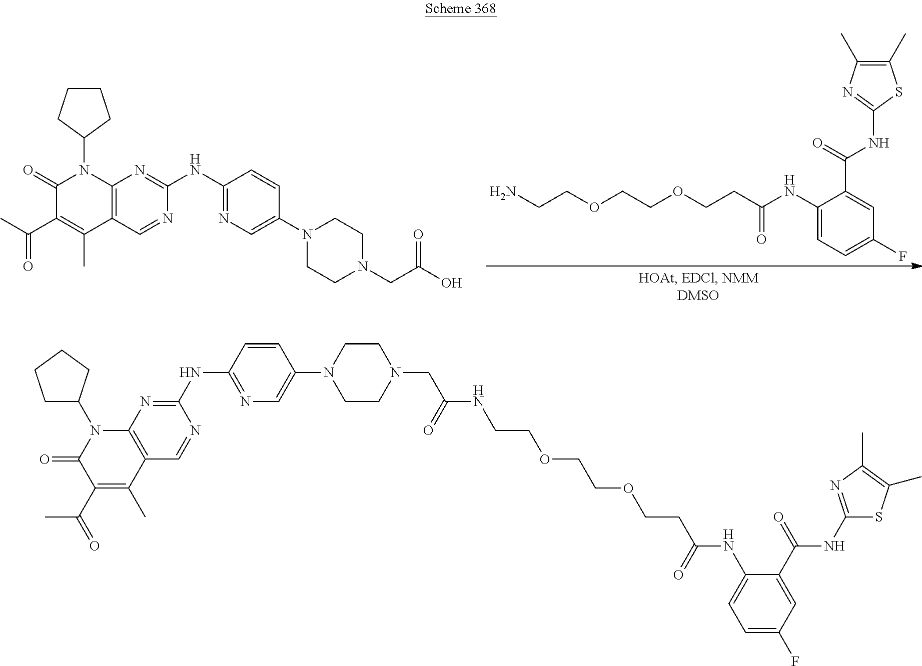 [SMILES: CC(=O)c1c(C)c2cnc(Nc3ccc(N4CCN(CC(=O)O)CC4)cn3)nc2n(C2CCCC2)c1=O ; Cc1nc(NC(=O)c2cc(F)ccc2NC(=O)CCOCCOCCN)sc1C ; CC(=O)c1c(C)c2cnc(Nc3ccc(N4CCN(CC(=O)NCCOCCOCCC(=O)Nc5ccc(F)cc5C(=O)Nc5nc(C)c(C)s5)CC4)cn3)nc2n(C2CCCC2)c1=O]

CPD-152 was synthesized following the standard procedure for preparing CPD-042 (1.18 mg, 12% yield). MS (ESI) m/z=912.8 [M+H]⁺.

### Example 369. 2-(4-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-4-oxobutanamido)-N-(4,5-dimethylthiazol-2-yl)benzamide (CPD-153)

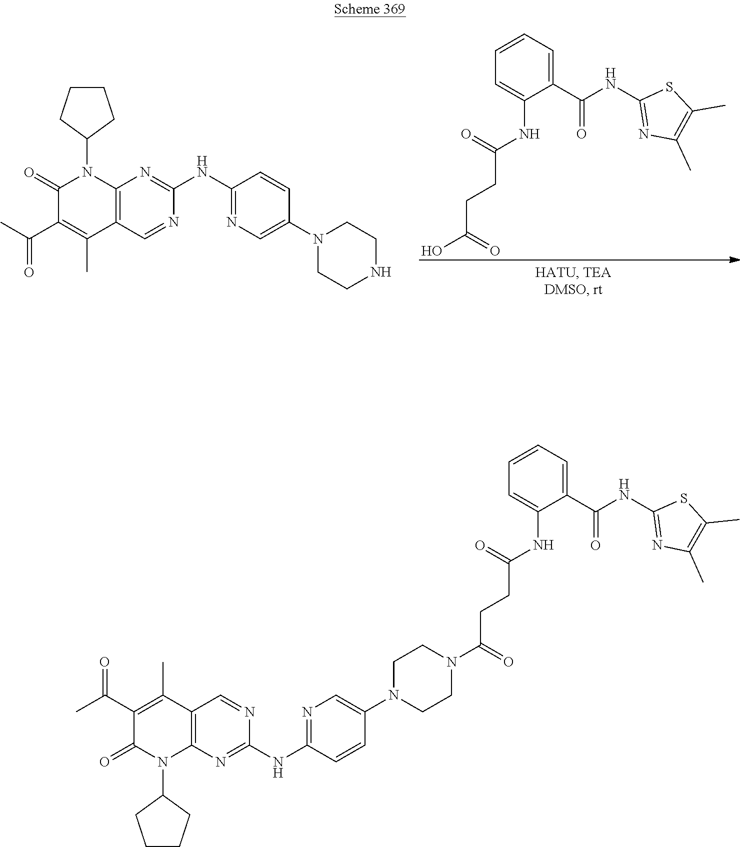 [SMILES: CC(=O)c1c(C)c2cnc(Nc3ccc(N4CCNCC4)cn3)nc2n(C2CCCC2)c1=O ; Cc1nc(NC(=O)c2ccccc2NC(=O)CCC(=O)O)sc1C ; CC(=O)c1c(C)c2cnc(Nc3ccc(N4CCN(C(=O)CCC(=O)Nc5ccccc5C(=O)Nc5nc(C)c(C)s5)CC4)cn3)nc2n(C2CCCC2)c1=O]

CPD-153 was synthesized following the standard procedure for preparing CPD-146 (0.6 mg, 3% yield). MS (ESI) m/z=777.5 [M+H]⁺.

### Example 370. 3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)-N-(2-(((4,5-dimethylthiazol-2-yl)amino)methyl)phenyl)propanamide (CPD-154)

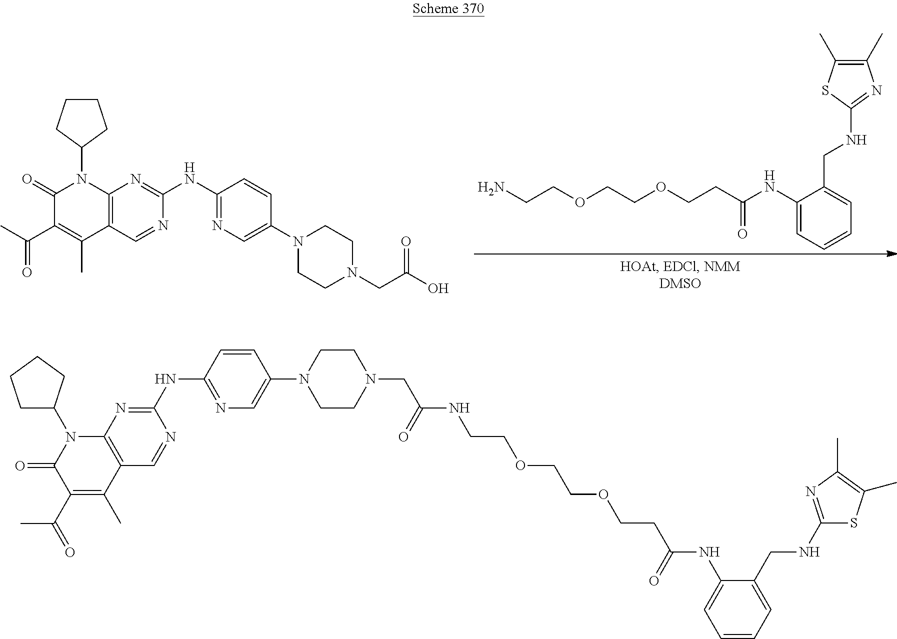 [SMILES: CC(=O)c1c(C)c2cnc(Nc3ccc(N4CCN(CC(=O)O)CC4)cn3)nc2n(C2CCCC2)c1=O ; Cc1nc(NCc2ccccc2NC(=O)CCOCCOCCN)sc1C ; CC(=O)c1c(C)c2cnc(Nc3ccc(N4CCN(CC(=O)NCCOCCOCCC(=O)Nc5ccccc5CNc5nc(C)c(C)s5)CC4)cn3)nc2n(C2CCCC2)c1=O]

CPD-154 was synthesized following the standard procedure for preparing CPD-042 (2.25 mg, 26% yield). MS (ESI) m/z=881.1 [M+H]⁺.

### Example 371. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-4-(dimethylamino)-N-(4,5-dimethylthiazol-2-yl)benzamide (CPD-155)

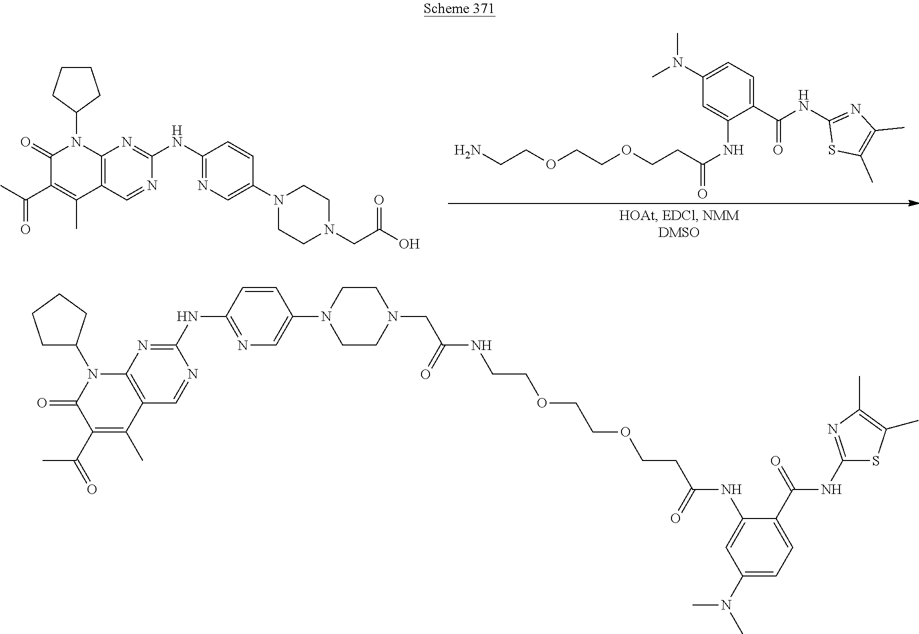 [SMILES: CC(=O)c1c(C)c2cnc(Nc3ccc(N4CCN(CC(=O)O)CC4)cn3)nc2n(C2CCCC2)c1=O ; Cc1nc(NC(=O)c2ccc(N(C)C)cc2NC(=O)CCOCCOCCN)sc1C ; CC(=O)c1c(C)c2cnc(Nc3ccc(N4CCN(CC(=O)NCCOCCOCCC(=O)Nc5cc(N(C)C)ccc5C(=O)Nc5nc(C)c(C)s5)CC4)cn3)nc2n(C2CCCC2)c1=O]

CPD-155 was synthesized following the standard procedure for preparing CPD-042 (2.79 mg, 27% yield). MS (ESI) m/z=938.0 [M+H]⁺.

### Example 372. 2-(6-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-6-oxohexanamido)-N-(4,5-dimethylthiazol-2-yl)benzamide (CPD-156)

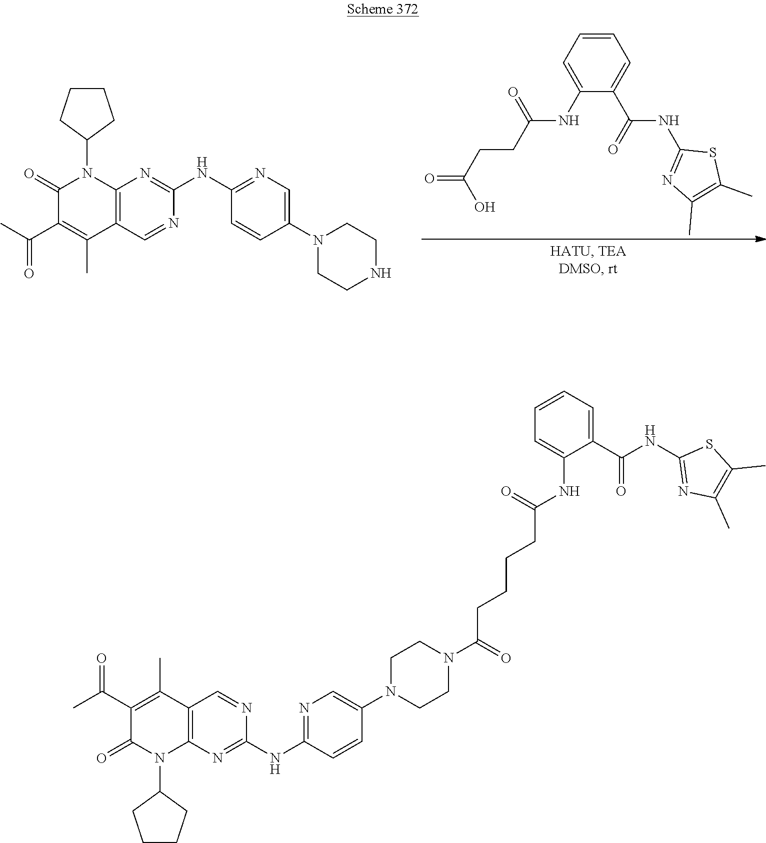 [SMILES: CC(=O)c1c(C)c2cnc(Nc3ccc(N4CCNCC4)cn3)nc2n(C2CCCC2)c1=O ; Cc1nc(NC(=O)c2ccccc2NC(=O)CCC(=O)O)sc1C ; CC(=O)c1c(C)c2cnc(Nc3ccc(N4CCN(C(=O)CCCCC(=O)Nc5ccccc5C(=O)Nc5nc(C)c(C)s5)CC4)cn3)nc2n(C2CCCC2)c1=O]

CPD-156 was synthesized following the standard procedure for preparing CPD-146 (1.0 mg, 5% yield). MS (ESI) m/z=805.8 [M+H]⁺.

### Example 373. 2-(7-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-7-oxoheptanamido)-N-(4,5-dimethylthiazol-2-yl)benzamide (CPD-157)

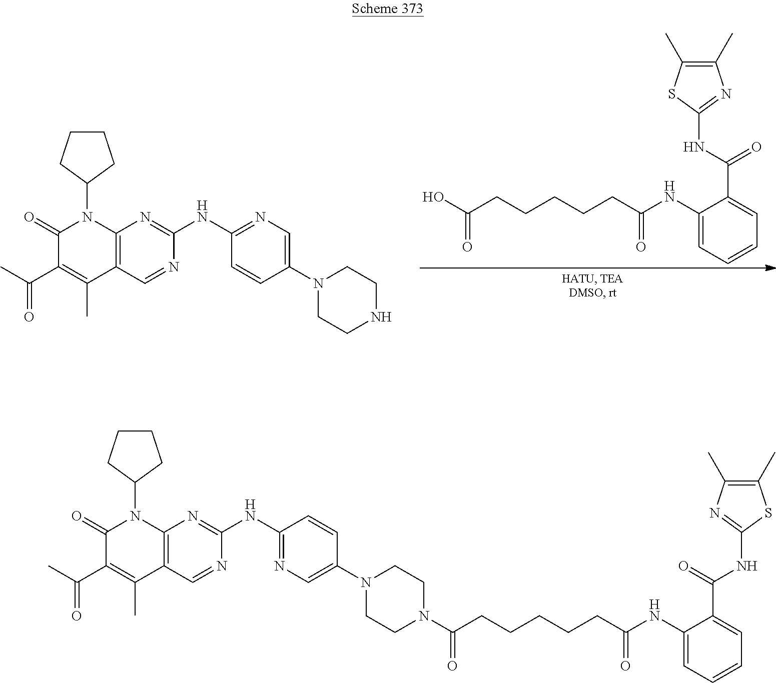 [SMILES: CC(=O)c1c(C)c2cnc(Nc3ccc(N4CCNCC4)cn3)nc2n(C2CCCC2)c1=O ; Cc1nc(NC(=O)c2ccccc2NC(=O)CCCCCC(=O)O)sc1C ; CC(=O)c1c(C)c2cnc(Nc3ccc(N4CCN(C(=O)CCCCCC(=O)Nc5ccccc5C(=O)Nc5nc(C)c(C)s5)CC4)cn3)nc2n(C2CCCC2)c1=O]

CPD-157 was synthesized following the standard procedure for preparing CPD-146 (1.4 mg, yield: 7%). MS (ESI) m/z=819.8 [M+H]⁺.

### Example 374. 2-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-3-oxopropanamido)-N-(4,5-dimethylthiazol-2-yl)benzamide (CPD-158)

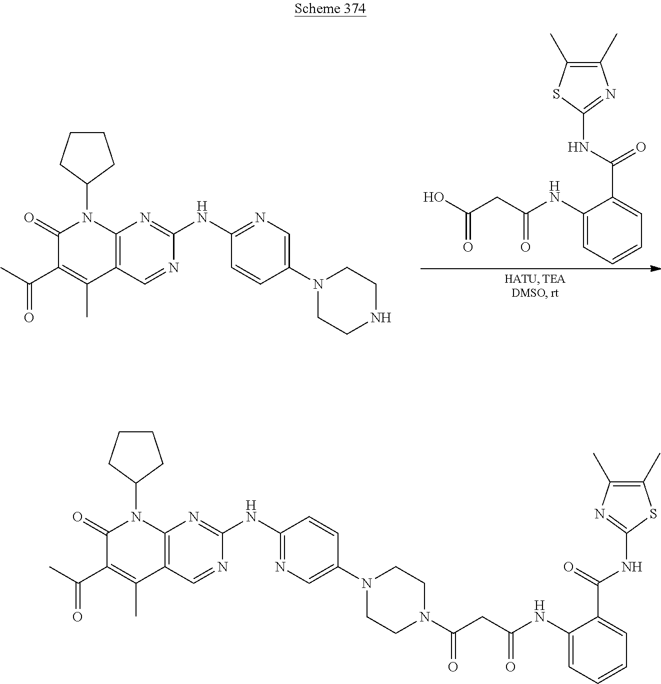 [SMILES: CC(=O)c1c(C)c2cnc(Nc3ccc(N4CCNCC4)cn3)nc2n(C2CCCC2)c1=O ; Cc1nc(NC(=O)c2ccccc2NC(=O)CC(=O)O)sc1C ; CC(=O)c1c(C)c2cnc(Nc3ccc(N4CCN(C(=O)CC(=O)Nc5ccccc5C(=O)Nc5nc(C)c(C)s5)CC4)cn3)nc2n(C2CCCC2)c1=O]

CPD-158 was synthesized following the standard procedure for preparing CPD-146 (0.5 mg, 2% yield). MS (ESI) m/z=763.8 [M+H]⁺.

### Example 375. 2-(5-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-5-oxopentanamido)-N-(4,5-dimethylthiazol-2-yl)benzamide (CPD-159)

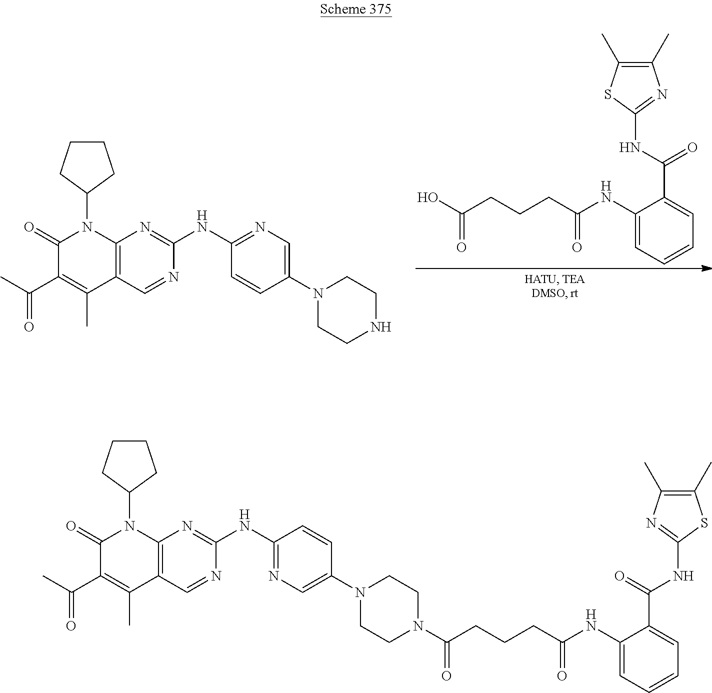 [SMILES: CC(=O)c1c(C)c2cnc(Nc3ccc(N4CCNCC4)cn3)nc2n(C2CCCC2)c1=O ; Cc1nc(NC(=O)c2ccccc2NC(=O)CCCC(=O)O)sc1C ; CC(=O)c1c(C)c2cnc(Nc3ccc(N4CCN(C(=O)CCCC(=O)Nc5ccccc5C(=O)Nc5nc(C)c(C)s5)CC4)cn3)nc2n(C2CCCC2)c1=O]

CPD-159 was synthesized following the standard procedure for preparing CPD-146 (1.0 mg, 5% yield). MS (ESI) m/z=791.8 [M+H]⁺.

### Example 376. 2-(9-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-9-oxononanamido)-N-(4,5-dimethylthiazol-2-yl)benzamide (CPD-160)

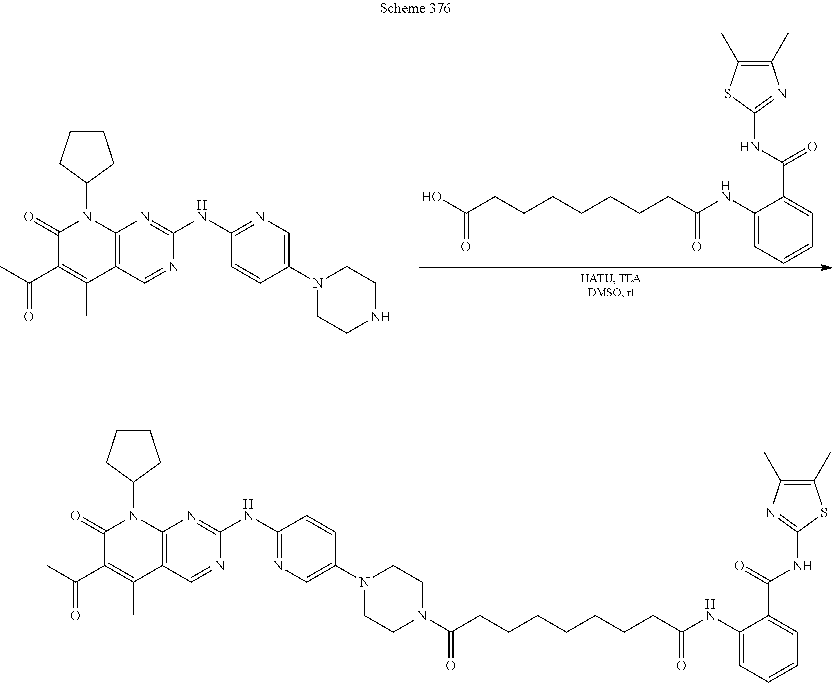 [SMILES: CC(=O)c1c(C)c2cnc(Nc3ccc(N4CCNCC4)cn3)nc2n(C2CCCC2)c1=O ; Cc1nc(NC(=O)c2ccccc2NC(=O)CCCCCCCC(=O)O)sc1C ; CC(=O)c1c(C)c2cnc(Nc3ccc(N4CCN(C(=O)CCCCCCCC(=O)Nc5ccccc5C(=O)Nc5nc(C)c(C)s5)CC4)cn3)nc2n(C2CCCC2)c1=O]

CPD-160 was synthesized following the standard procedure for preparing CPD-146 (4.6 mg, 23% yield) MS (ESI) m/z=847.9 [M+H]⁺.

### Example 377. 2-(8-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-8-oxooctanamido)-N-(4,5-dimethylthiazol-2-yl)benzamide (CPD-161)

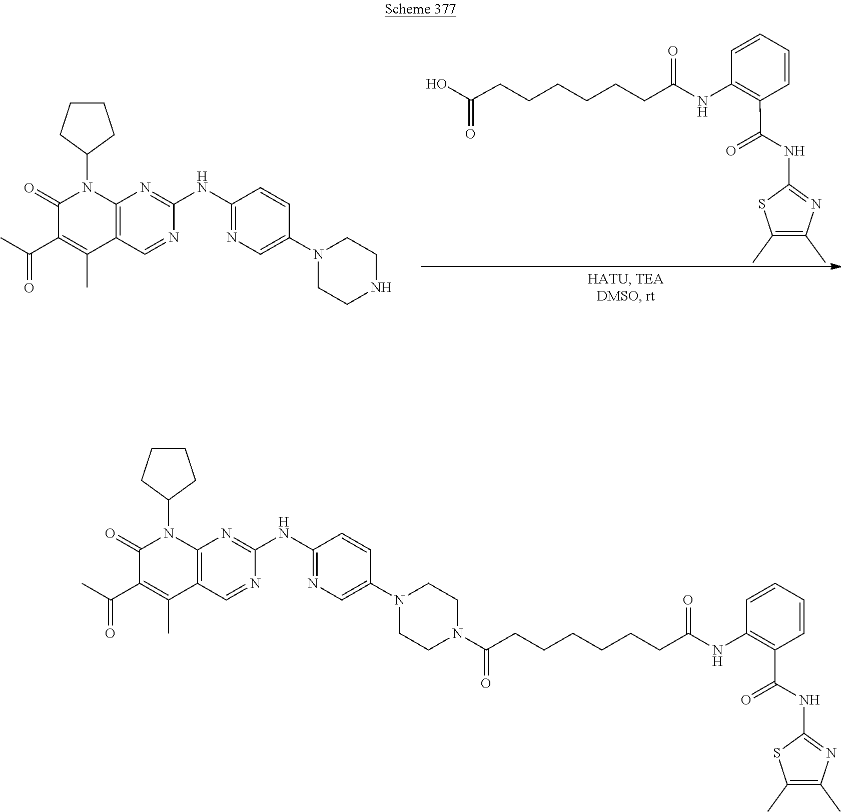 [SMILES: CC(=O)c1c(C)c2cnc(Nc3ccc(N4CCNCC4)cn3)nc2n(C2CCCC2)c1=O ; Cc1nc(NC(=O)c2ccccc2NC(=O)CCCCCCC(=O)O)sc1C ; CC(=O)c1c(C)c2cnc(Nc3ccc(N4CCN(C(=O)CCCCCCC(=O)Nc5ccccc5C(=O)Nc5nc(C)c(C)s5)CC4)cn3)nc2n(C2CCCC2)c1=O]

CPD-161 was synthesized following the standard procedure for preparing CPD-146 (8.8 mg, 43% yield). MS (ESI) m/z=833.8 [M+H]⁺.

### Example 378. 2-(10-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-10-oxodecanamido)-N-(4,5-dimethylthiazol-2-yl)benzamide (CPD-162)

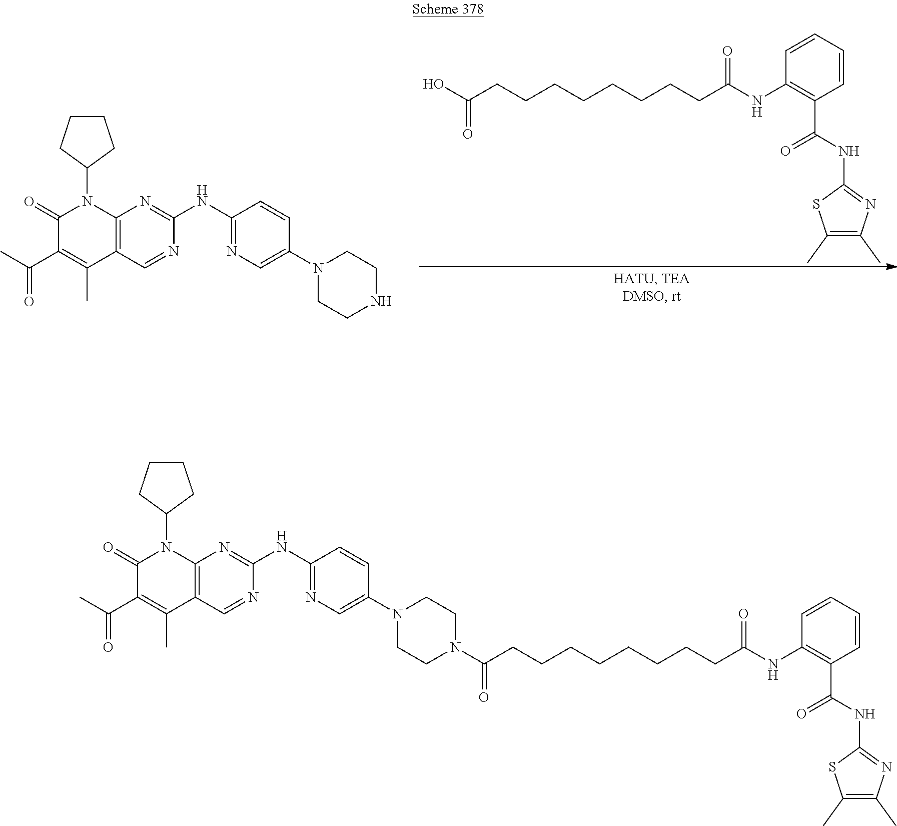 [SMILES: CC(=O)c1c(C)c2cnc(Nc3ccc(N4CCNCC4)cn3)nc2n(C2CCCC2)c1=O ; Cc1nc(NC(=O)c2ccccc2NC(=O)CCCCCCCCC(=O)O)sc1C ; CC(=O)c1c(C)c2cnc(Nc3ccc(N4CCN(C(=O)CCCCCCCCC(=O)Nc5ccccc5C(=O)Nc5nc(C)c(C)s5)CC4)cn3)nc2n(C2CCCC2)c1=O]

CPD-162 was synthesized following the standard procedure for preparing CPD-146 (9.6 mg, 48% yield). MS (ESI) m/z=861.8 [M+H]⁺.

### Example 379. 19-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-N-(2-((4,5-dimethylthiazol-2-yl)carbamoyl)phenyl)-19-oxo-4,7,10,13,16-pentaoxanonadecanamide (CPD-163)

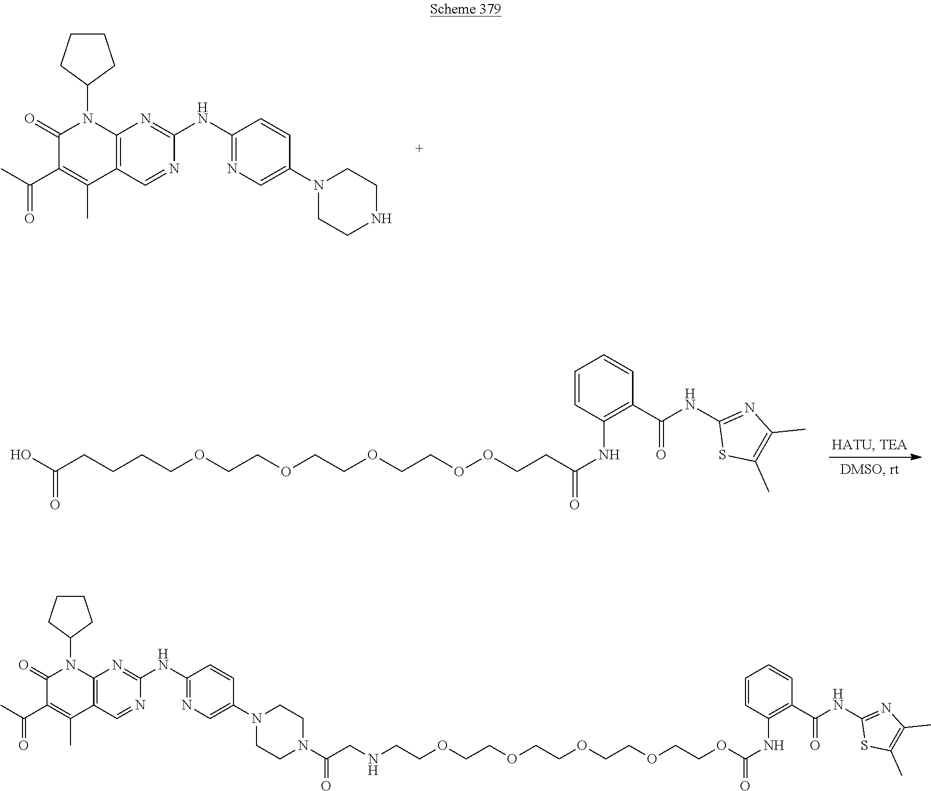

CPD-163 was synthesized following the standard procedure for preparing CPD-146 (6.3 mg, 36% yield). MS (ESI) m/z=997.8 [M+H]⁺.

### Example 380. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4,5-dimethylthiazol-2-yl)-4-(methylamino)benzamide (CPD-164)

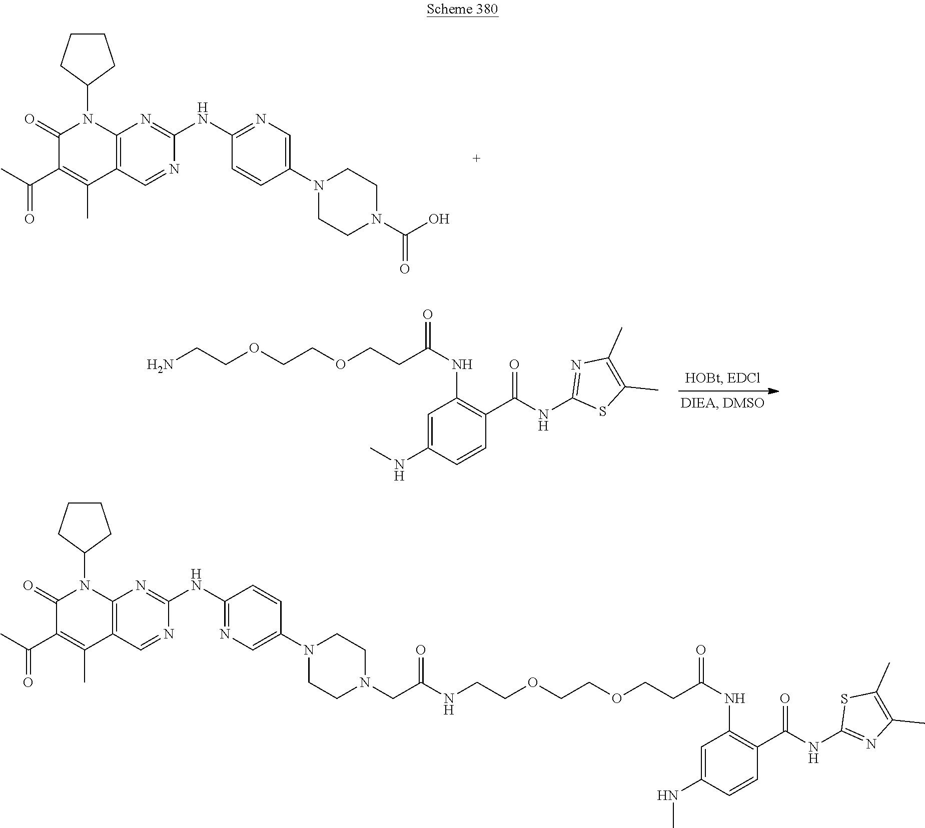 [SMILES: CC(=O)c1c(C)c2cnc(Nc3ccc(N4CCN(C(=O)O)CC4)cn3)nc2n(C2CCCC2)c1=O ; "+" ; CNc1ccc(C(=O)Nc2nc(C)c(C)s2)c(NC(=O)CCOCCOCCN)c1 ; CNc1ccc(C(=O)Nc2nc(C)c(C)s2)c(NC(=O)CCOCCOCCNC(=O)CN2CCN(c3ccc(Nc4ncc5c(C)c(C(C)=O)c(=O)n(C6CCCC6)c5n4)nc3)CC2)c1]

CPD-164 was synthesized following the standard procedure for preparing CPD-167 (10.32 mg, 55% yield). MS (ESI) m/z=924.0 [M+H]⁺.

### Example 381. 5-((4-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-4-oxobutyl)amino)-N-(4,5-dimethylthiazol-2-yl)-2-methylbenzamide (CPD-165)

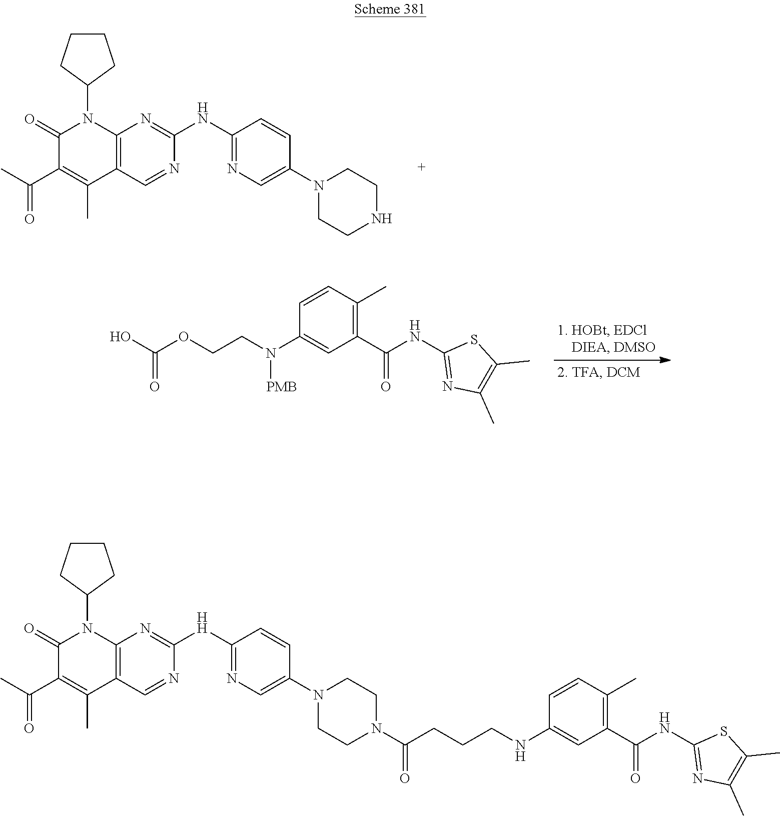

### Step 1. Synthesis of 5-((4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4-oxobutyl)(4-methoxybenzyl)amino)-N-(4,5-dimethylthiazol-2-yl)-2-methylbenzamide

To a solution of 2-(5-(piperazin-1-yl)pyridin-2-ylamino)-6-acetyl-8-cyclopentyl-5-methylpyrido[2,3-d]pyrimidin-7(8H)-one (15 mg, 33.5 μmol) and 4-(N-(3-(4,5-dimethylthiazol-2-ylcarbamoyl)-4-methylphenyl)-N-(4-methoxybenzyl)amino)butanoic acid (15.7 mg, 33.5 μmol) in DMSO (2.5 mL) were added EDCI (12.8 mg, 67.0 mmol), HOBt (9.1 mg, 67.0 mmol) and DIEA (43.0 mg, 335 μmol) at 0° C. The mixture was stirred at rt for 16 h. Upon completion, the reaction mixture was poured into water and extracted with EtOAc. The combined organic layers were concentrated and the resulting residue was purified by silica gel chromatography (DCM/MeOH=20:1) to provide the title compound (11.0 mg, 36% yield). MS (ESI) m/z=898.0 [M+H]⁺.

### Step 2. Synthesis of 5-((4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4-oxobutyl)amino)-N-(4,5-dimethylthiazol-2-yl)-2-methylbenzamide

To a solution of 5-((4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4-oxobutyl)(4-methoxybenzyl)amino)-N-(4,5-dimethylthiazol-2-yl)-2-methylbenzamide (11.0 mg, 12.2 μmol) in DCM (5 mL) was added TFA (2.5 mL). After the mixture was stirred at rt overnight, it was concentrated under reduced pressure. The residue was purified by reverse-phase chromatography to provide the title compound (7.43 mg, 6% yield). MS (ESI) m/z=777.8 [M+H]⁺.

### Example 382. 2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-N-(17-((4-(((4,5-dimethylthiazol-2-yl)amino)methyl)-3-methylphenyl)amino)-3,6,9,12,15-pentaoxaheptadecyl)acetamide (CPD-166)

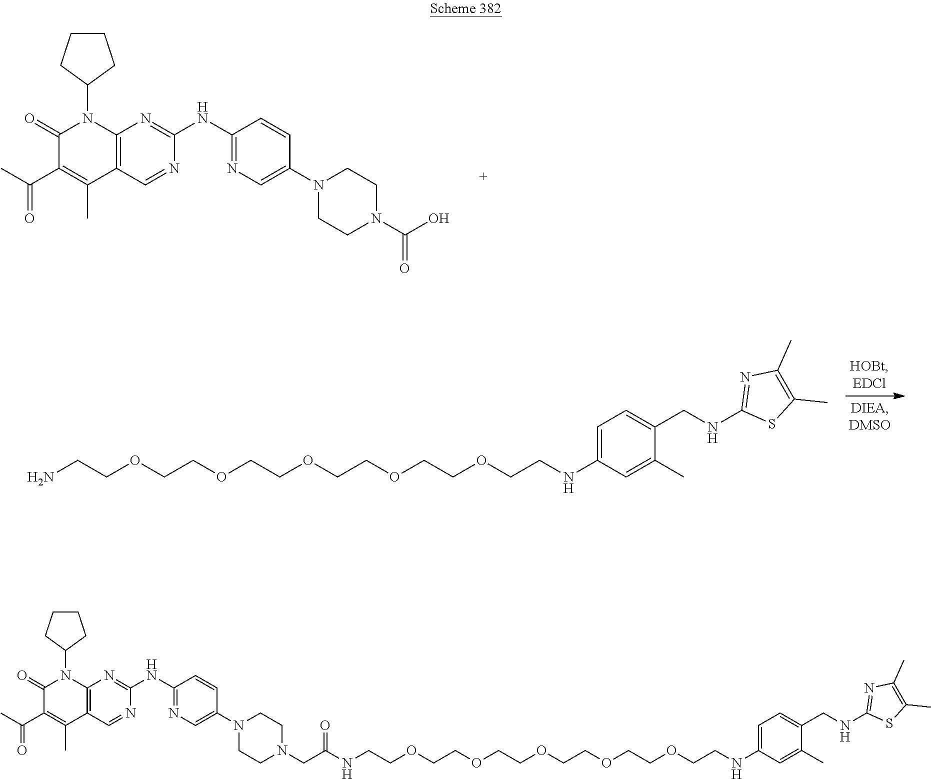

CPD-166 was synthesized following the standard procedure for preparing CPD-167 (1.26 mg, 6% yield). MS (ESI) m/z=999.0 [M+H]⁺.

### Example 383. 3-((8-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-8-oxooctyl)amino)-N-(4,5-dimethylthiazol-2-yl)-2-methylbenzamide (CPD-167)

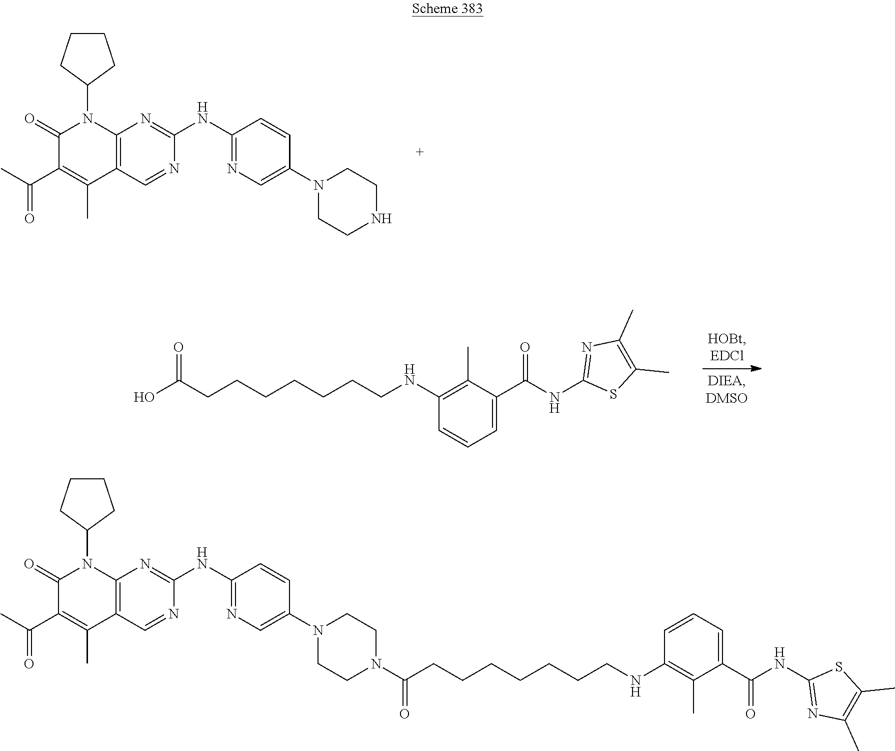 [SMILES: CC(=O)c1c(C)c2cnc(Nc3ccc(N4CCNCC4)cn3)nc2n(C2CCCC2)c1=O ; Cc1nc(NC(=O)c2cccc(NCCCCCCCC(=O)O)c2C)sc1C ; CC(=O)c1c(C)c2cnc(Nc3ccc(N4CCN(C(=O)CCCCCCCNc5cccc(C(=O)Nc6nc(C)c(C)s6)c5C)CC4)cn3)nc2n(C2CCCC2)c1=O]

To a solution of 6-acetyl-8-cyclopentyl-5-methyl-2-((5-(piperazin-1-yl)pyridin-2-yl)amino)pyrido[2,3-d]pyrimidin-7(8H)-one (10 mg, 22.34 μmol), 8-((3-((4,5-dimethylthiazol-2-yl)carbamoyl)-2-methylphenyl)amino)octanoic acid (BL1-120, 9.02 mg, 22.34 μmol) and DIEA (28.82 mg, 223.40 μmol) in DMSO (2.5 mL) was added EDCI (8.57 mg, 44.68 μmol) and HOBt (6.08 mg, 44.68 μmol) at 0° C. The mixture was stirred at rt for 16 h. Upon completion, the reaction mixture was poured into water and extracted with DCM. The combined organic layers were concentrated and the resulting residue was purified by silica gel chromatography (DCM:MeOH=20:1) to provide the title compound (10.35 mg, 56% yield). MS (ESI) m/z=833.9 [M+H]⁺.

### Example 384. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-4-(butylamino)-N-(4,5-dimethylthiazol-2-yl)benzamide (CPD-168)

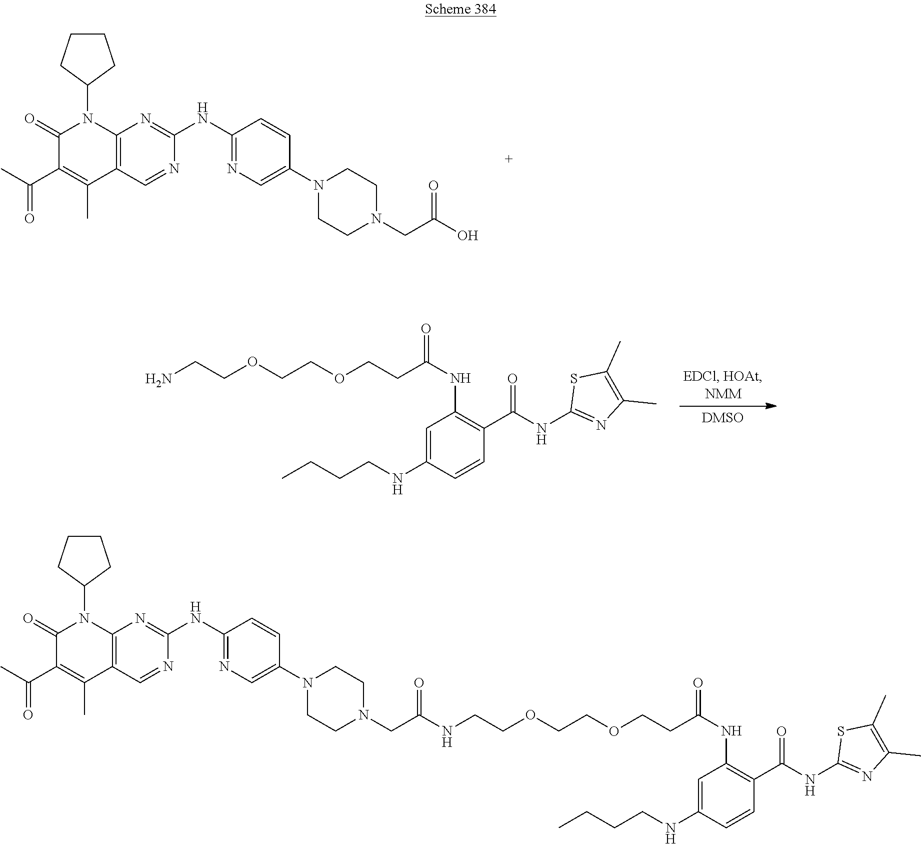 [SMILES: CC(=O)c1c(C)c2cnc(Nc3ccc(N4CCN(CC(=O)O)CC4)cn3)nc2n(C2CCCC2)c1=O ; CCCCNc1ccc(C(=O)Nc2nc(C)c(C)s2)c(NC(=O)CCOCCOCCN)c1 ; CCCCNc1ccc(C(=O)Nc2nc(C)c(C)s2)c(NC(=O)CCOCCOCCNC(=O)CN2CCN(c3ccc(Nc4ncc5c(C)c(C(C)=O)c(=O)n(C6CCCC6)c5n4)nc3)CC2)c1]

CPD-168 was synthesized following the standard procedure for preparing CPD-042 (4.1 mg, 43% yield) as a yellow solid. MS (ESI) m/z=966.0 [M+H]⁺.

### Example 385. 2-(7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptanamido)-N-(4,5-dimethylthiazol-2-yl)benzamide (CPD-169)

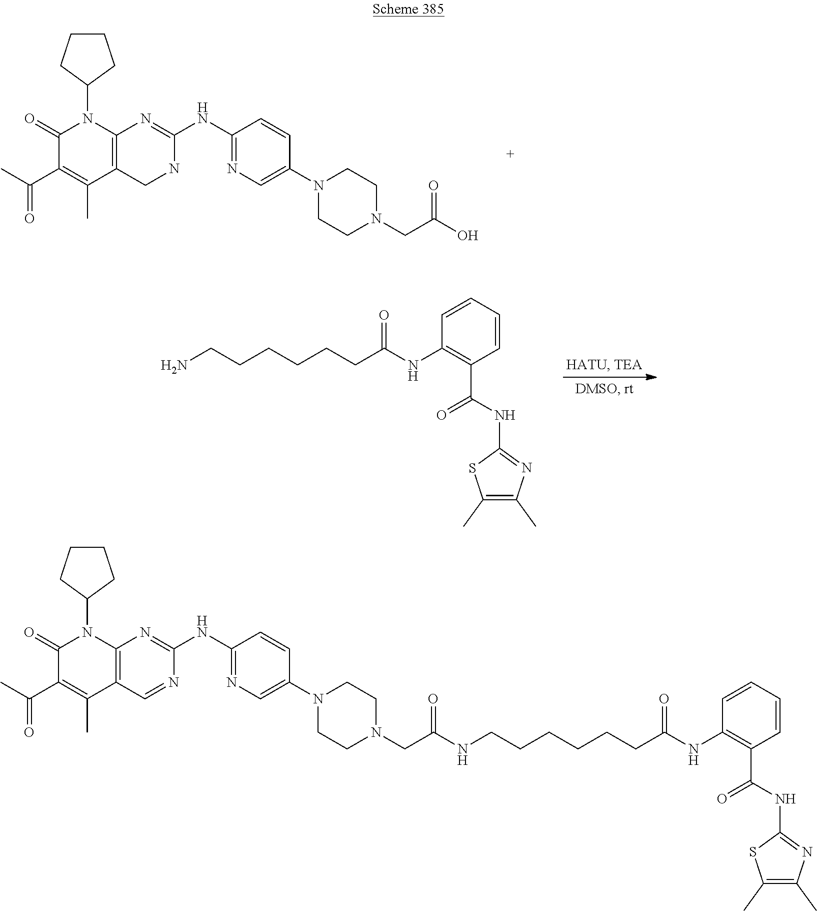 [SMILES: CC(=O)c1c(C)c2c(n(C3CCCC3)c1=O)N=C(Nc1ccc(N3CCN(CC(=O)O)CC3)cn1)NC2 ; Cc1nc(NC(=O)c2ccccc2NC(=O)CCCCCCN)sc1C ; CC(=O)c1c(C)c2cnc(Nc3ccc(N4CCN(CC(=O)NCCCCCCC(=O)Nc5ccccc5C(=O)Nc5nc(C)c(C)s5)CC4)cn3)nc2n(C2CCCC2)c1=O]

CPD-169 was synthesized following the standard procedure for preparing CPD-146 (2.6 mg, 11% yield). MS (ESI) m/z=862.9 [M+H]⁺.

### Example 386. 2-(1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15-tetraoxa-3-azaoctadecan-18-amido)-N-(4,5-dimethylthiazol-2-yl)benzamide (CPD-170)

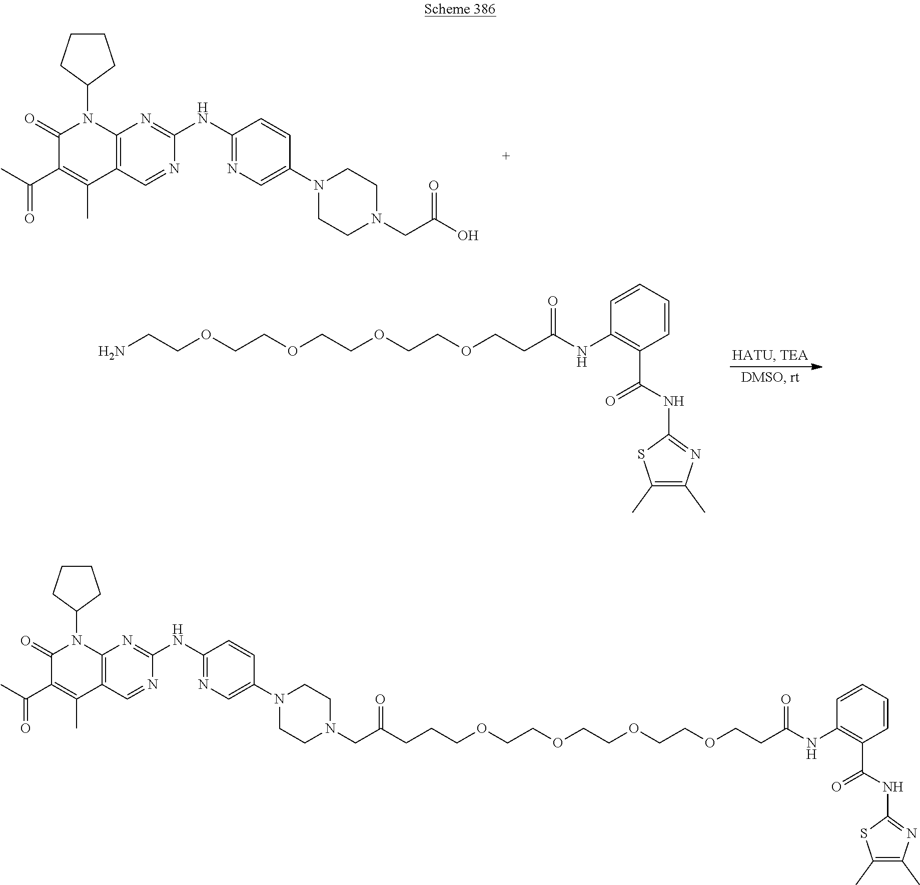

CPD-170 was synthesized following the standard procedure for preparing CPD-146 (2.9 mg, 15% yield). MS (ESI) m/z=982.9 [M+H]⁺.

### Example 387. 2-(4-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)butanamido)-N-(4,5-dimethylthiazol-2-yl)benzamide (CPD-171)

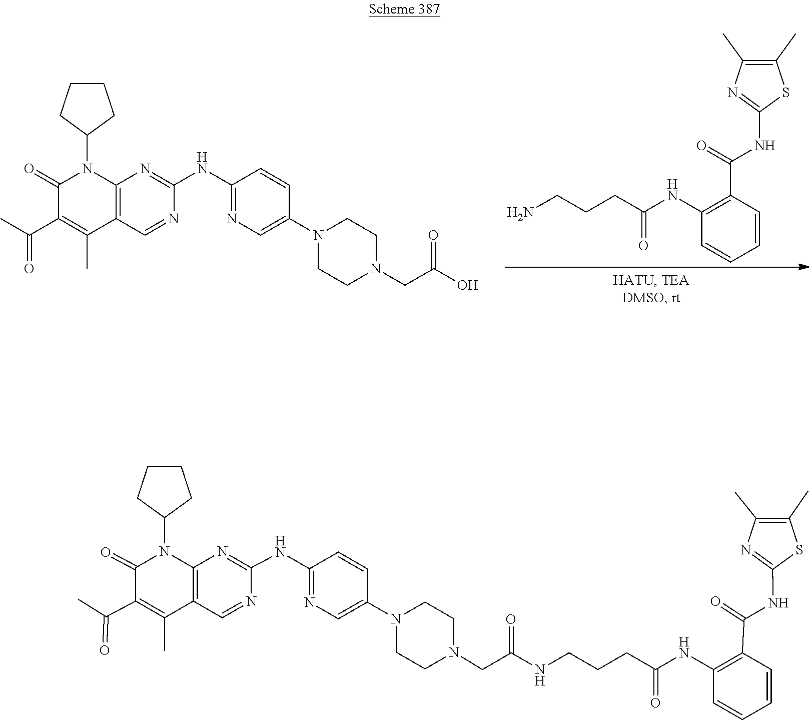 [SMILES: CC(=O)c1c(C)c2cnc(Nc3ccc(N4CCN(CC(=O)O)CC4)cn3)nc2n(C2CCCC2)c1=O ; Cc1nc(NC(=O)c2ccccc2NC(=O)CCCN)sc1C ; CC(=O)c1c(C)c2cnc(Nc3ccc(N4CCN(CC(=O)NCCCC(=O)Nc5ccccc5C(=O)Nc5nc(C)c(C)s5)CC4)cn3)nc2n(C2CCCC2)c1=O]

CPD-171 was synthesized following the standard procedure for preparing CPD-146 (2.8 mg, 11% yield). MS (ESI) m/z=820.8 [M+H]⁺.

### Example 388. 2-(6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)hexanamido)-N-(4,5-dimethylthiazol-2-yl)benzamide (CPD-172)

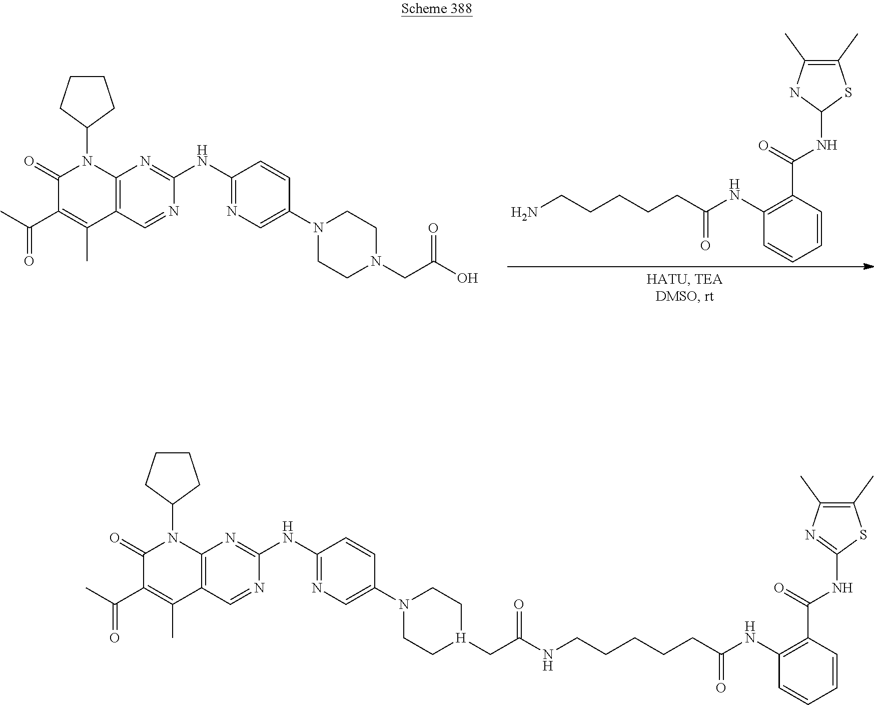

CPD-172 was synthesized following the standard procedure for preparing CPD-146 (2.5 mg, 11% yield). MS (ESI) m/z=848.9 [M+H]⁺.

### Example 389. 2-(1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18,21-hexaoxa-3-azatetracosan-24-amido)-N-(4,5-dimethylthiazol-2-yl)benzamide (CPD-173)

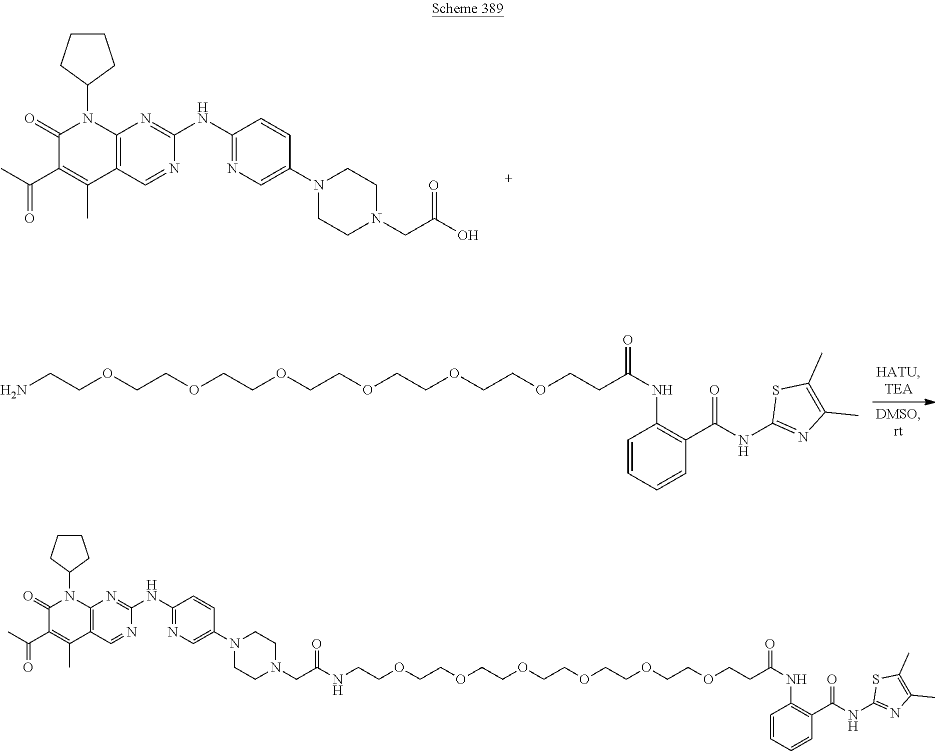

CPD-173 was synthesized following the standard procedure for preparing CPD-146 (2.6 mg, 14% yield). MS (ESI) m/z=1071.0 [M+H]⁺.

### Example 390. 2-(5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentanamido)-N-(4,5-dimethylthiazol-2-yl)benzamide (CPD-174)

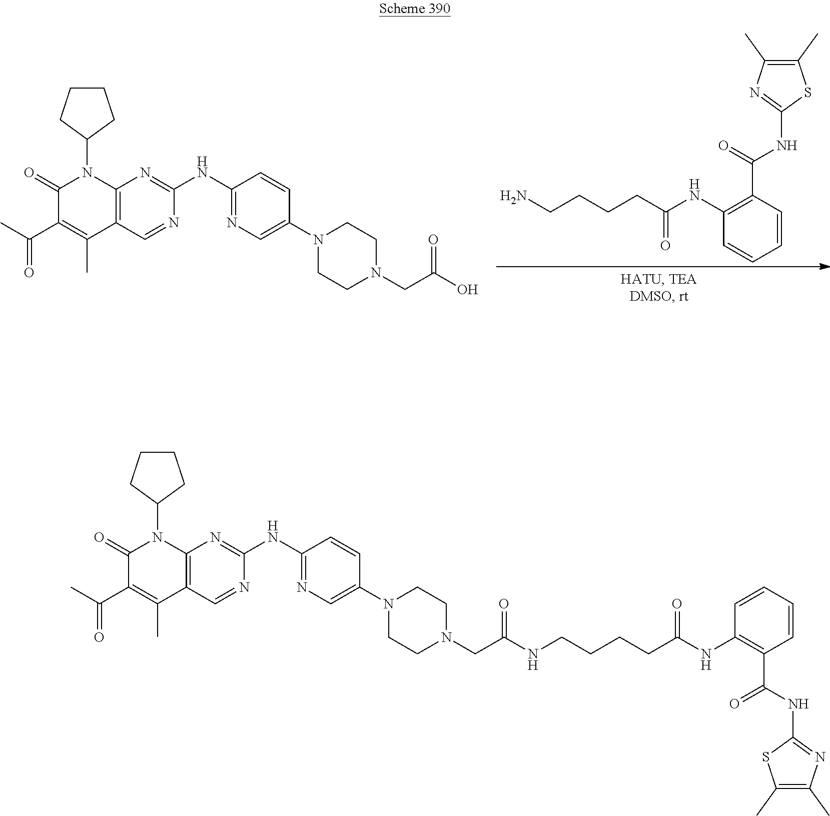 [SMILES: CC(=O)c1c(C)c2cnc(Nc3ccc(N4CCN(CC(=O)O)CC4)cn3)nc2n(C2CCCC2)c1=O ; Cc1nc(NC(=O)c2ccccc2NC(=O)CCCCN)sc1C ; CC(=O)c1c(C)c2cnc(Nc3ccc(N4CCN(CC(=O)NCCCCC(=O)Nc5ccccc5C(=O)Nc5nc(C)c(C)s5)CC4)cn3)nc2n(C2CCCC2)c1=O]

CPD-174 was synthesized following the standard procedure for preparing CPD-146 (1.4 mg, 6% yield). MS (ESI) m/z=834.8 [M+H]⁺.

### Example 391. 2-(1-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxo-6,9,12,15,18-pentaoxa-3-azahenicosan-21-amido)-N-(4,5-dimethylthiazol-2-yl)benzamide (CPD-175)

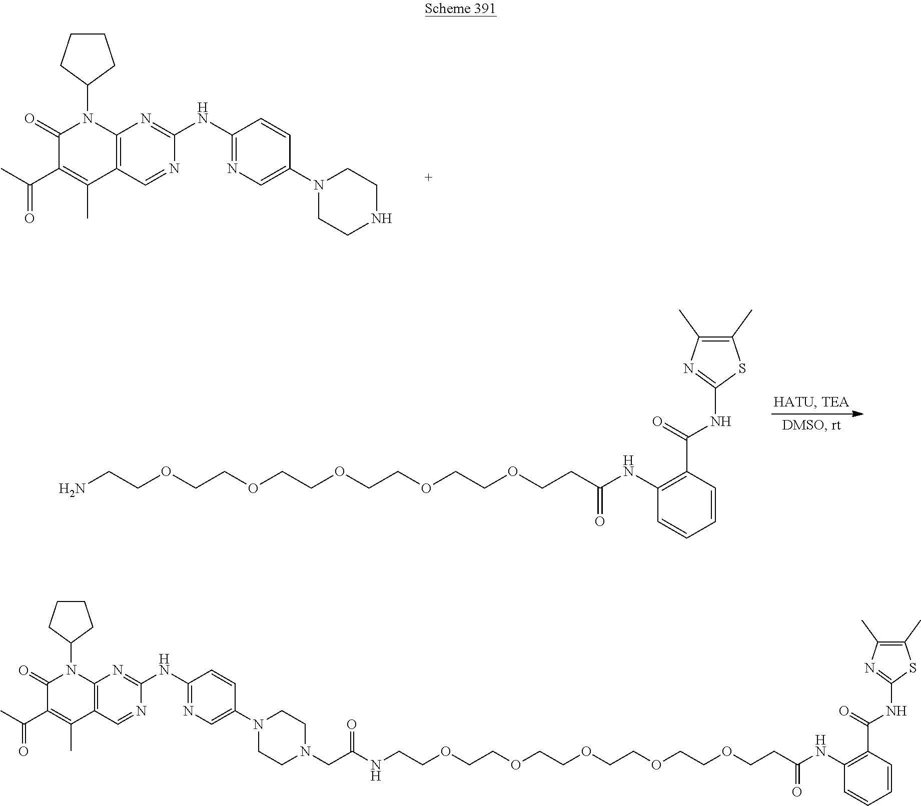

CPD-175 was synthesized following the standard procedure for preparing CPD-146 (2.2 mg, 12% yield). MS (ESI) m/z=1027.1 [M+H]⁺.

### Example 392. 16-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-N-(2-((4,5-dimethylthiazol-2-yl)carbamoyl)phenyl)-16-oxo-4,7,10,13-tetraoxahexadecanamide (CPD-176)

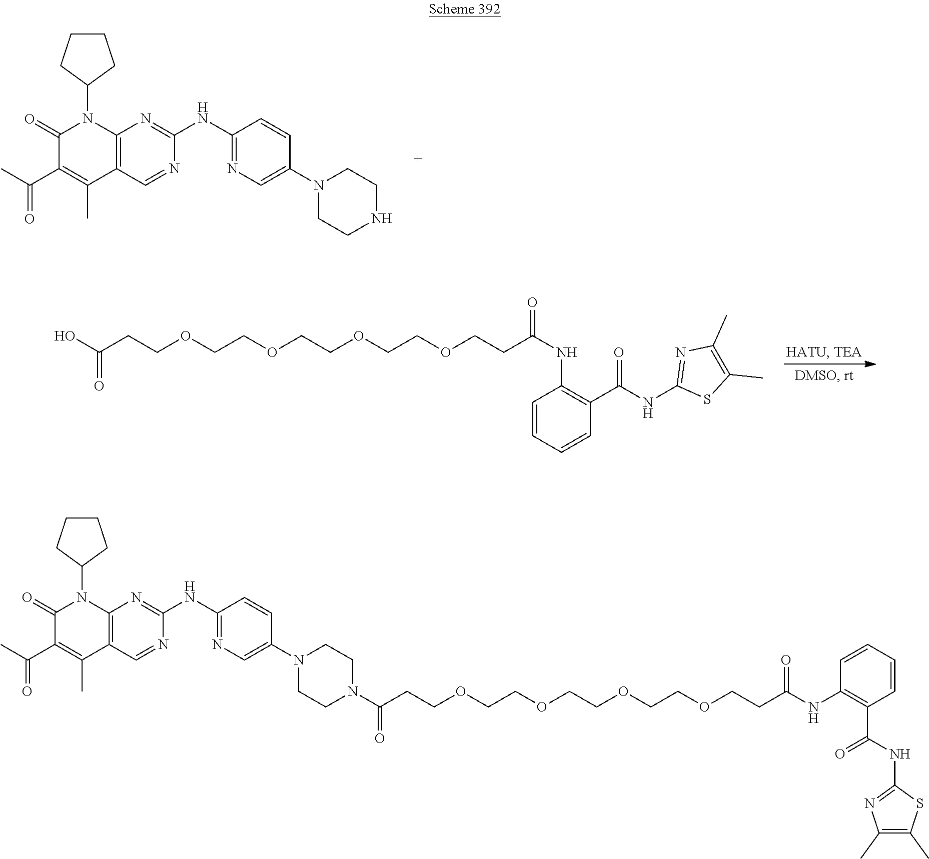

CPD-176 was synthesized following the standard procedure for preparing CPD-146 (2.7 mg, 3% yield). MS (ESI) m/z=953.9 [M+H]⁺.

### Example 393. 2-(12-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)dodecanamido)-N-(4,5-dimethylthiazol-2-yl)benzamide (CPD-177)

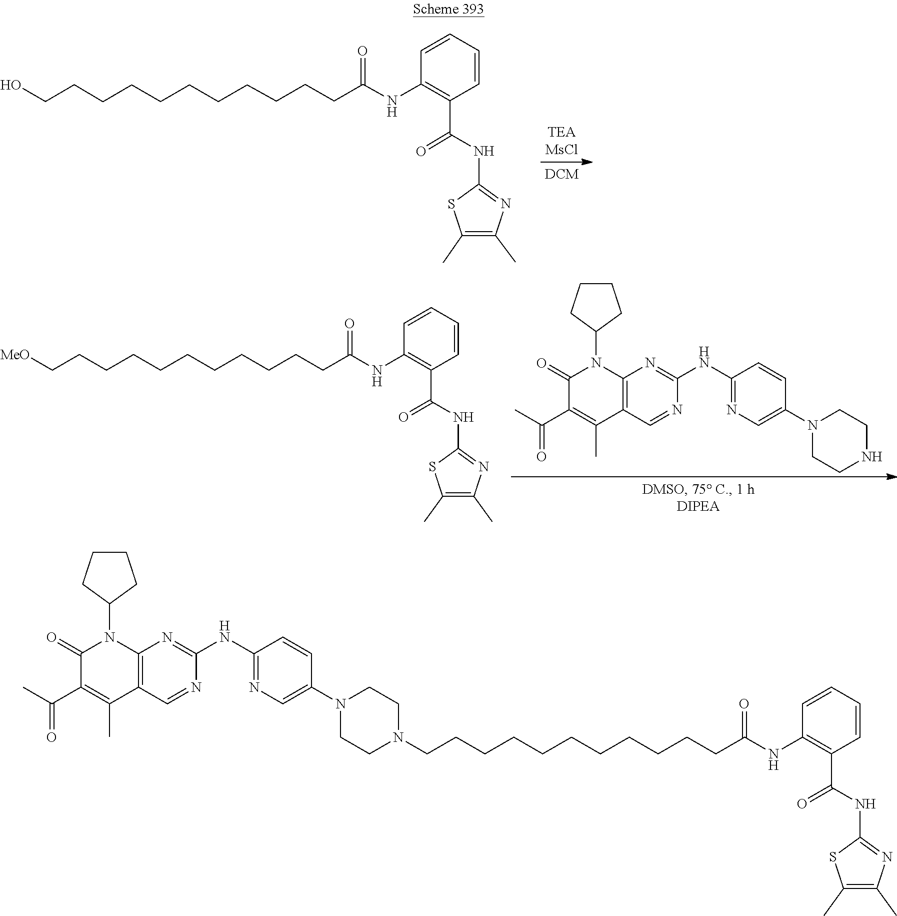

### Step 1. Synthesis of 12-((2-((4,5-dimethylthiazol-2-yl)carbamoyl)phenyl)amino)-12-oxododecyl methanesulfonate

To a stirred mixture of N-(4,5-dimethylthiazol-2-yl)-2-(12-hydroxydodecanoylamino)benzamide (20 mg, 44.9 μmol) and TEA (13 mg, 134.6 μmol) in DCM (0.5 mL) was added MsCl (10 mg, 89.7 μmol). The reaction mixture was stirred at rt for 1 h. Upon completion, the mixture was concentrated, and the residue was purified by prep-TLC (DCM/MeOH=20:1) to provide the title compound (20 mg, 85% yield) as a bright oil.

### Step 2. Synthesis of 2-(12-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)dodecanamido)-N-(4,5-dimethylthiazol-2-yl)benzamide

To a mixture of 12-((2-((4,5-dimethylthiazol-2-yl)carbamoyl)phenyl)amino)-12-oxododecyl methanesulfonate (15 mg, 28.6 μmol) and 6-acetyl-8-cyclopentyl-5-methyl-2-((5-(piperazin-1-yl)pyridin-2-yl)amino)pyrido[2,3-d]pyrimidin-7(8H)-one (15.38 mg, 34.4 μmol) in DMSO (0.5 mL) was added DIPEA (11 mg, 85.9 μmol). The reaction was stirred at 70° C. for 1 h. The solution was purified by reverse phase-chromatography (0-70% MeCN in H₂O) to provide the title compound (1.2 mg, 5% yield) as a yellow solid. MS (ESI) m/z=875.9 [M+H]⁺.

### Example 394. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)ethoxy)ethoxy)ethoxy)propanamido)-N-(4,5-dimethylthiazol-2-yl)benzamide (CPD-178)

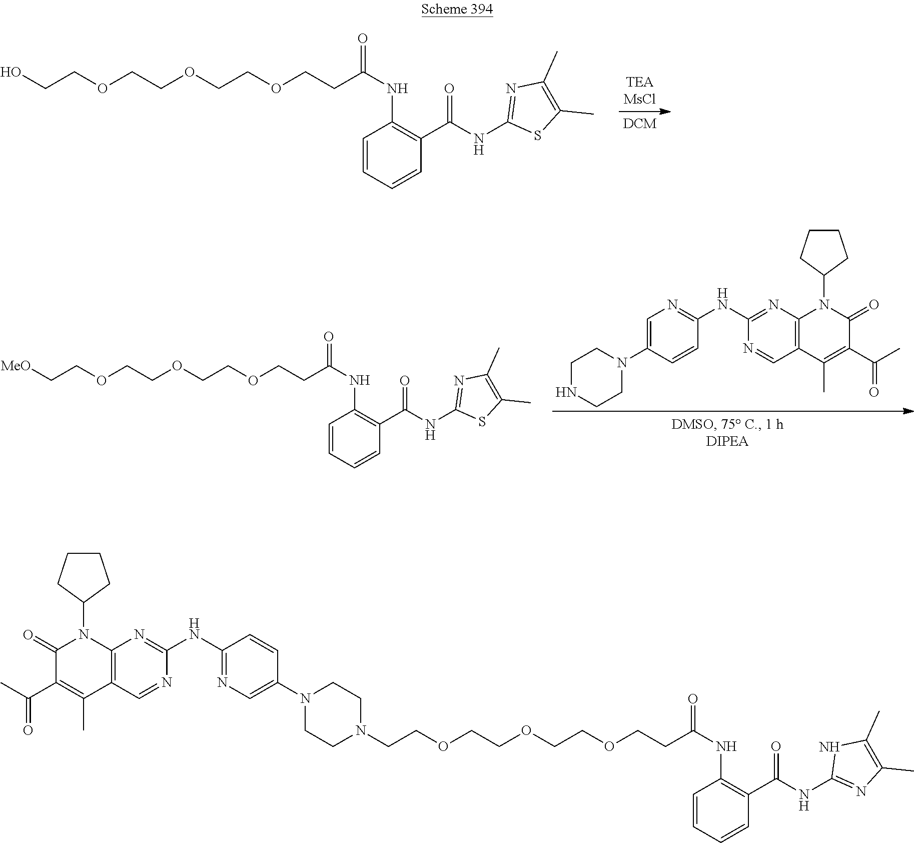

CPD-178 was synthesized following the standard procedure for preparing CPD-177 (2.0 mg, 12% yield). MS (ESI) m/z=881.8 [M+H]⁺.

### Example 395. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4,5-dimethylthiazol-2-yl)cyclohexane-1-carboxamide (CPD-179)

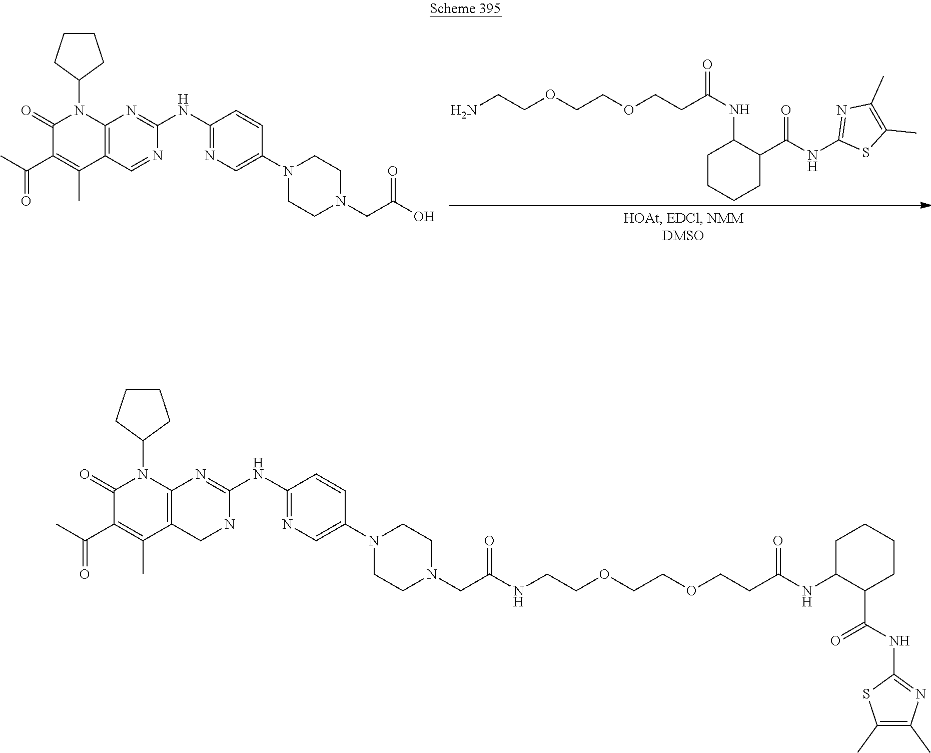 [SMILES: CC(=O)c1c(C)c2cnc(Nc3ccc(N4CCN(CC(=O)O)CC4)cn3)nc2n(C2CCCC2)c1=O ; Cc1nc(NC(=O)C2CCCCC2NC(=O)CCOCCOCCN)sc1C ; CC(=O)c1c(C)c2c(n(C3CCCC3)c1=O)N=C(Nc1ccc(N3CCN(CC(=O)NCCOCCOCCC(=O)NC4CCCCC4C(=O)Nc4nc(C)c(C)s4)CC3)cn1)NC2]

CPD-179 was synthesized following the standard procedure for preparing CPD-042 (3.23 mg, 36% yield). MS (ESI) m/z=901.0 [M+H]⁺.

### Example 396. 2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)acetamido)-N-(4,5-dimethylthiazol-2-yl)benzamide (CPD-180)

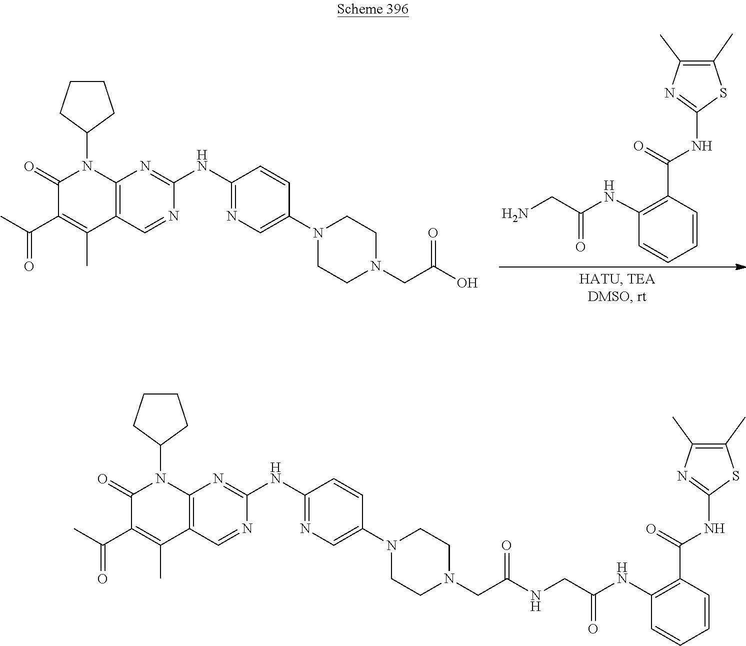 [SMILES: CC(=O)c1c(C)c2cnc(Nc3ccc(N4CCN(CC(=O)O)CC4)cn3)nc2n(C2CCCC2)c1=O ; Cc1nc(NC(=O)c2ccccc2NC(=O)CN)sc1C ; CC(=O)c1c(C)c2cnc(Nc3ccc(N4CCN(CC(=O)NCC(=O)Nc5ccccc5C(=O)Nc5nc(C)c(C)s5)CC4)cn3)nc2n(C2CCCC2)c1=O]

CPD-180 was synthesized following the standard procedure for preparing CPD-146 (3.2 mg, 12% yield). MS (ESI) m/z=792.8 [M+H]⁺.

### Example 397. 2-(3-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-3-oxopropoxy)propanamido)-N-(4,5-dimethylthiazol-2-yl)benzamide (CPD-181)

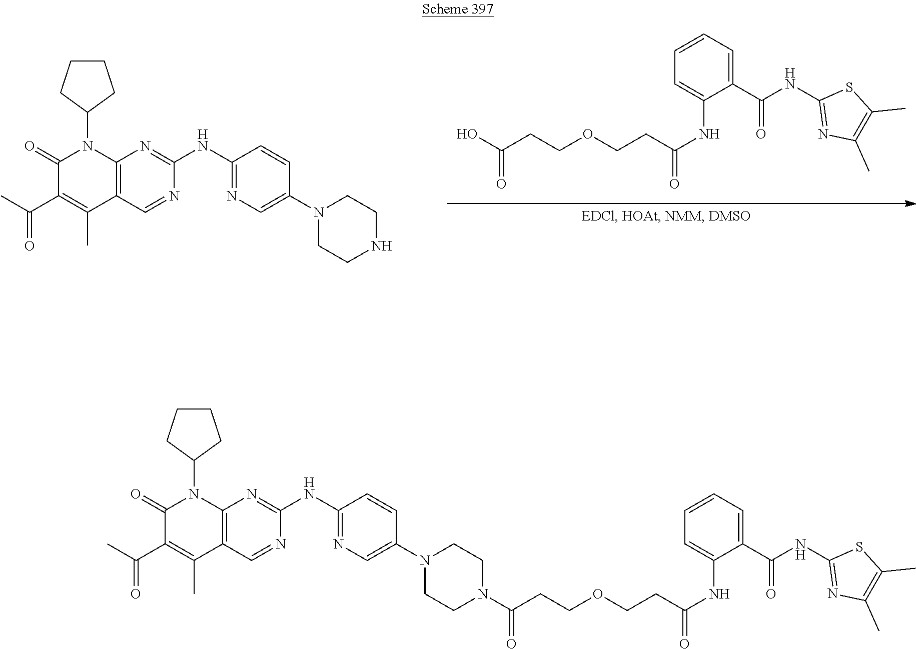 [SMILES: CC(=O)c1c(C)c2cnc(Nc3ccc(N4CCNCC4)cn3)nc2n(C2CCCC2)c1=O ; Cc1nc(NC(=O)c2ccccc2NC(=O)CCOCCC(=O)O)sc1C ; CC(=O)c1c(C)c2cnc(Nc3ccc(N4CCN(C(=O)CCOCCC(=O)Nc5ccccc5C(=O)Nc5nc(C)c(C)s5)CC4)cn3)nc2n(C2CCCC2)c1=O]

CPD-181 was synthesized following the standard procedure for preparing CPD-146 (3.6 mg, 20% yield). MS (ESI) m/z=822.0 [M+H]⁺.

### Example 398. 22-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-N-(2-((4,5-dimethylthiazol-2-yl)carbamoyl)phenyl)-22-oxo-4,7,10,13,16,19-hexaoxadocosanamide (CPD-182)

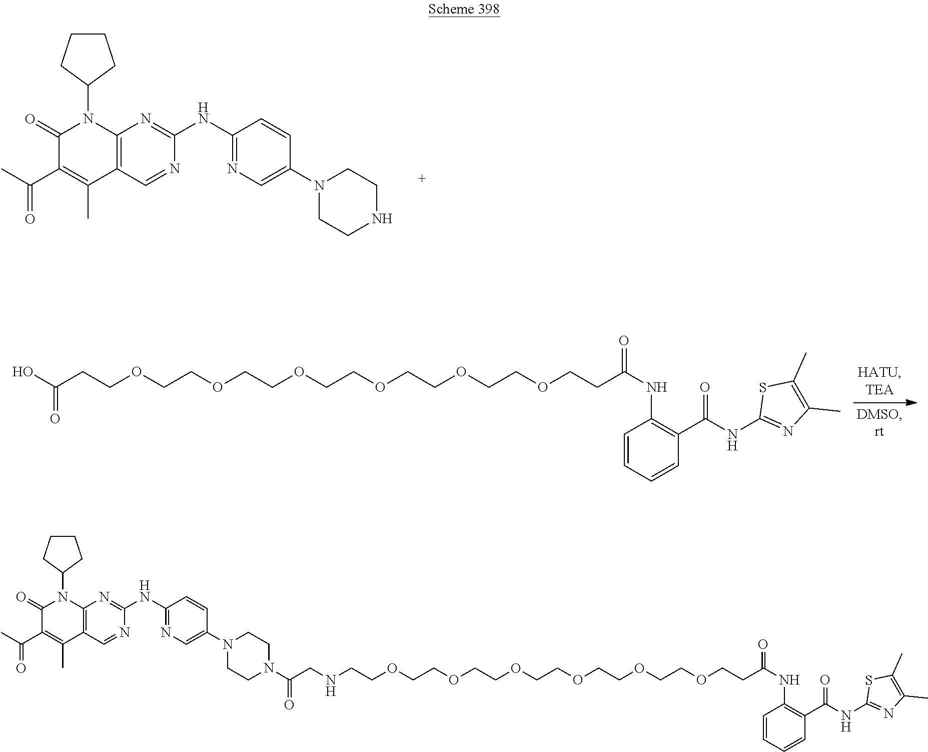

CPD-182 was synthesized following the standard procedure for preparing CPD-146 (7.5 mg, 44% yield). MS (ESI) m/z=1064.1 [M+H]⁺.

### Example 399. 2-((3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propyl)amino)-N-(4,5-dimethylthiazol-2-yl)benzamide (CPD-267)

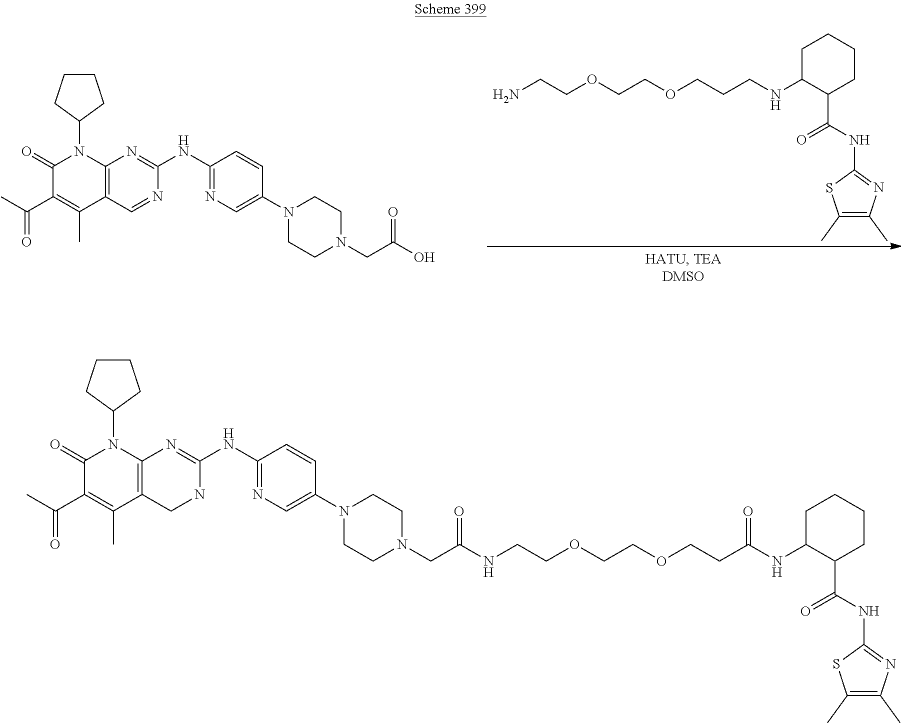 [SMILES: CC(=O)c1c(C)c2cnc(Nc3ccc(N4CCN(CC(=O)O)CC4)cn3)nc2n(C2CCCC2)c1=O ; Cc1nc(NC(=O)C2CCCCC2NCCCOCCOCCN)sc1C ; CC(=O)c1c(C)c2c(n(C3CCCC3)c1=O)N=C(Nc1ccc(N3CCN(CC(=O)NCCOCCOCCC(=O)NC4CCCCC4C(=O)Nc4nc(C)c(C)s4)CC3)cn1)NC2]

CPD-267 was synthesized following the standard procedure for preparing CPD-146 (4.0 mg, 18% yield). MS (ESI) m/z=881.0 [M+H]⁺.

### Example 400. 2-((9-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)nonyl)amino)-N-(4,5-dimethylthiazol-2-yl)benzamide (CPD-268)

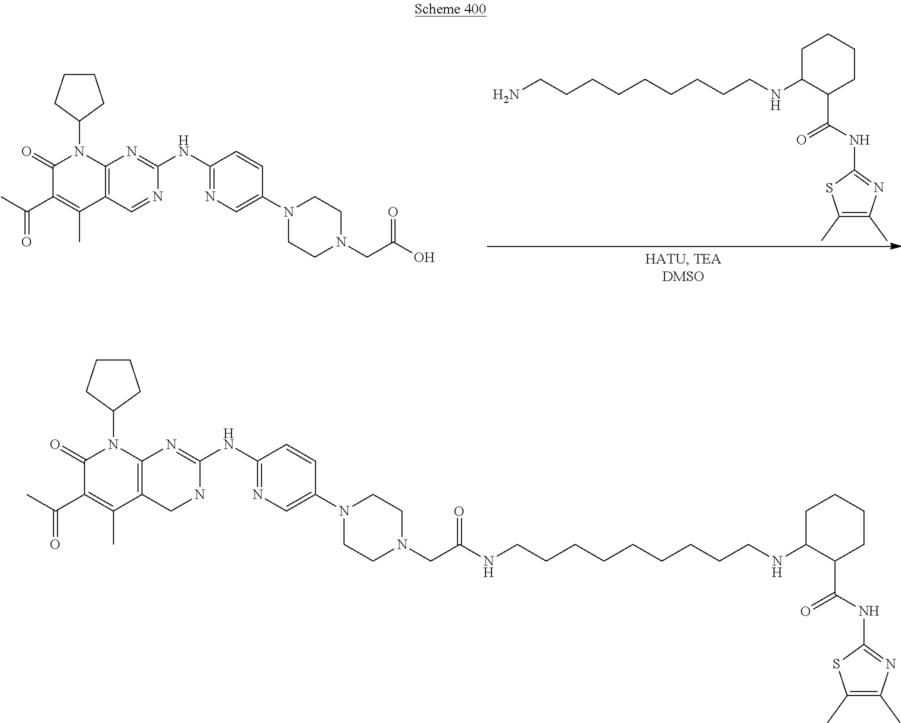 [SMILES: CC(=O)c1c(C)c2cnc(Nc3ccc(N4CCN(CC(=O)O)CC4)cn3)nc2n(C2CCCC2)c1=O ; Cc1nc(NC(=O)C2CCCCC2NCCCCCCCCCN)sc1C ; CC(=O)c1c(C)c2c(n(C3CCCC3)c1=O)N=C(Nc1ccc(N3CCN(CC(=O)NCCCCCCCCCNC4CCCCC4C(=O)Nc4nc(C)c(C)s4)CC3)cn1)NC2]

CPD-268 was synthesized following the standard procedure for preparing CPD-146 (1.4 mg, 2% yield). MS (ESI) m/z=876.9 [M+H]⁺.

### Example 401. (S)-2-(5-(2-(4-(4-Chlorophenyl)-2,3,9-trimethyl-6H-thieno[3,2-f][1,2,4]triazolo[4,3-a][1,4]diazepin-6-yl)acetamido)pentanamido)-N-(4,5-dimethylthiazol-2-yl)benzamide (CPD-219)

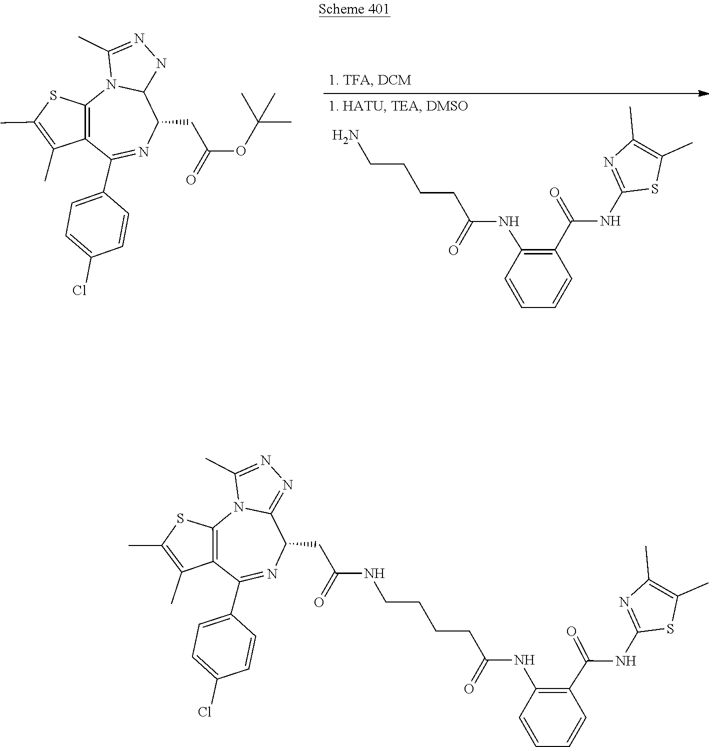

### Step 1. Synthesis of (S)-2-(4-(4-chlorophenyl)-2,3,9-trimethyl-6H-thieno[3,2-f][1,2,4]triazolo[4,3-a][1,4]diazepin-6-yl)acetic acid

To a solution of tert-butyl (S)-2-(4-(4-chlorophenyl)-2,3,9-trimethyl-6H-thieno[3,2-f][1,2,4]triazolo[4,3-a][1,4]diazepin-6-yl)acetate (100 mg, 218 μmol) in DCM (1 mL) was added TFA (1 mL). The mixture was stirred at rt for 1 h, then concentrated to provide the title compound (80 mg, 91% yield) as a yellow solid. MS (ESI) m/z=401.2 [M+H]⁺.

### Step 2. Synthesis of (S)-2-(5-(2-(4-(4-chlorophenyl)-2,3,9-trimethyl-6H-thieno[3,2-f][1,2,4]triazolo[4,3-a][1,4]diazepin-6-yl)acetamido)pentanamido)-N-(4,5-dimethylthiazol-2-yl)benzamide

To a mixture of 2-(5-aminopentanoylamino)-N-(4,5-dimethylthiazol-2-yl)benzamide (BL1-169, 10 mg, 29 μmol) and (S)-2-(4-(4-chlorophenyl)-2,3,9-trimethyl-6H-thieno[3,2-f][1,2,4]triazolo[4,3-a][1,4]diazepin-6-yl)acetic acid (14 mg, 35 μmol) in DMSO (0.5 mL) were added HATU (22 mg, 58 μmol) and TEA (9 mg, 86 μmol). After the reaction mixture was stirred at rt for 30 min, it was purified by reverse-phase chromatography (0-70% MeCN in H₂O) to provide the title compound (1.5 mg, 7% yield) as a yellow solid. MS (ESI) m/z=729.7 [M+H]⁺.

### Example 402. (S)-3-((2-(2-(4-(4-Chlorophenyl)-2,3,9-trimethyl-6H-thieno[3,2-f][1,2,4]triazolo[4,3-a][1,4]diazepin-6-yl)acetamido)ethyl)amino)-N-(4,5-dimethylthiazol-2-yl)-2-methylbenzamide (CPD-220)

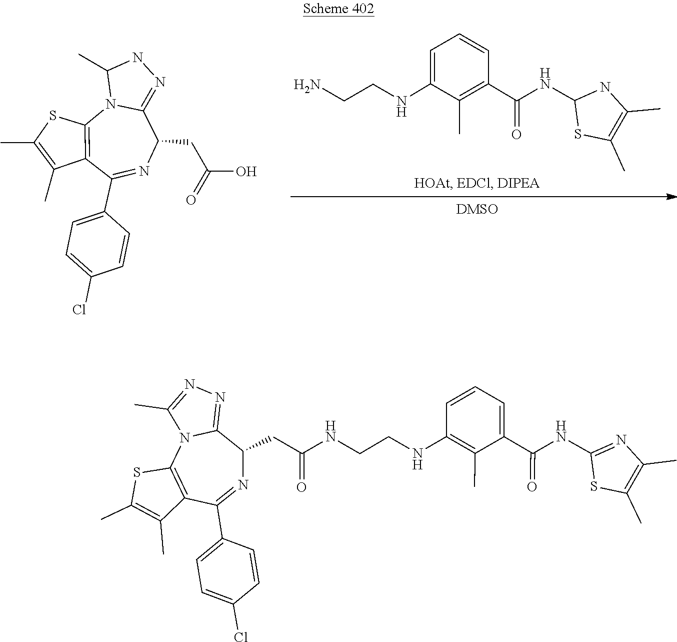

To a mixture of (S)-2-(4-(4-chlorophenyl)-2,3,9-trimethyl-6H-thieno[3,2-f][1,2,4]triazolo[4,3-a][1,4]diazepin-6-yl)acetic acid (5 mg, 12.5 μmol) and 3-((2-aminoethyl)amino)-N-(4,5-dimethylthiazol-2-yl)-2-methylbenzamide (3.7 mg, 12.5 μmol) in DMSO (1 mL) were added HOAt (5.34 mg, 25 μmol), EDCI (7.58 mg, 25 μmol) and DIPEA (8 mg, 62.5 μmol, 9.9 μL). After the resulting mixture was stirred at 25° C. for 1 h, it was purified by reverse-phase chromatography to provide the title compound (2.68 mg, 31% yield) as an off-white solid. MS (ESI) m/z=687.7 [M+H]⁺.

### Example 403. (S)-3-((3-(2-(4-(4-Chlorophenyl)-2,3,9-trimethyl-6H-thieno[3,2-f][1,2,4]triazolo[4,3-a][1,4]diazepin-6-yl)acetamido)propyl)amino)-N-(4,5-dimethylthiazol-2-yl)-2-methylbenzamide (CPD-221)

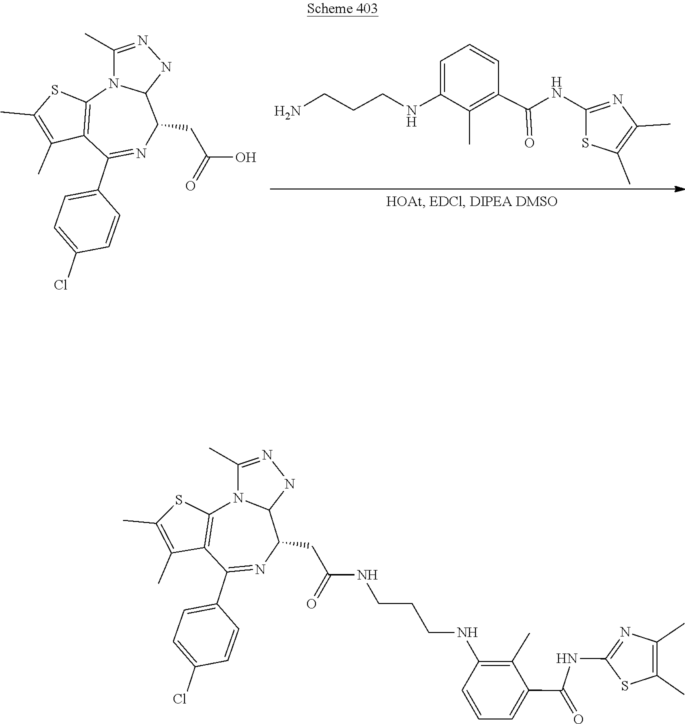

CPD-221 was synthesized following the standard procedure for preparing CPD-220 (2.15 mg, 25% yield). MS (ESI) m/z=701.6 [M+H]⁺.

### Example 404. (S)-3-((4-(2-(4-(4-Chlorophenyl)-2,3,9-trimethyl-6H-thieno[3,2-f][1,2,4]triazolo[4,3-a][1,4]diazepin-6-yl)acetamido)butyl)amino)-N-(4,5-dimethylthiazol-2-yl)-2-methylbenzamide (CPD-222)

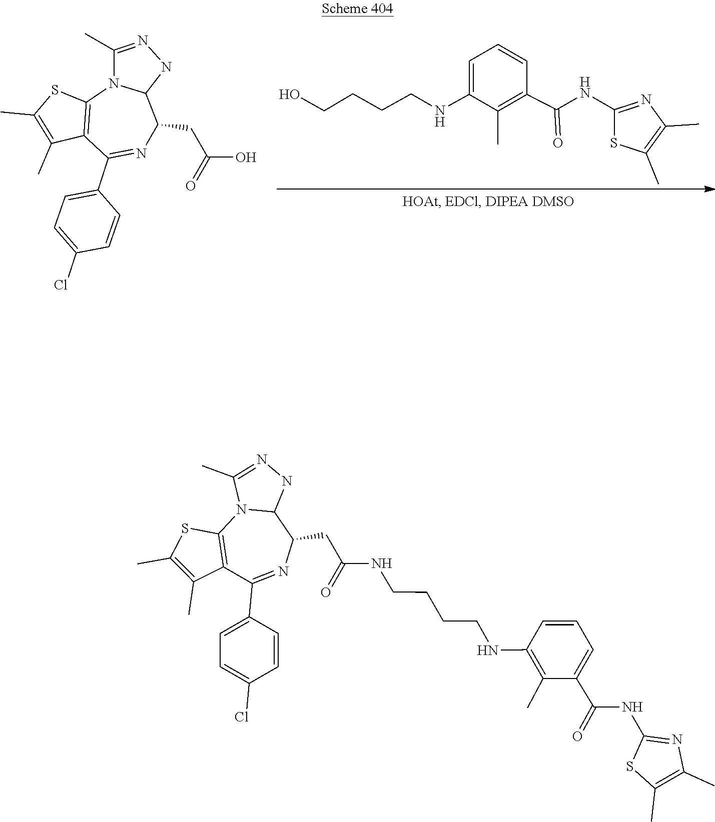

CPD-222 was synthesized following the standard procedure for preparing CPD-220 (3.07 mg, 34% yield). MS (ESI) m/z=715.7 [M+H]⁺.

### Example 405. (S)-3-((5-(2-(4-(4-Chlorophenyl)-2,3,9-trimethyl-6H-thieno[3,2-f][1,2,4]triazolo[4,3-a][1,4]diazepin-6-yl)acetamido)pentyl)amino)-N-(4,5-dimethylthiazol-2-yl)-2-methylbenzamide (CPD-223)

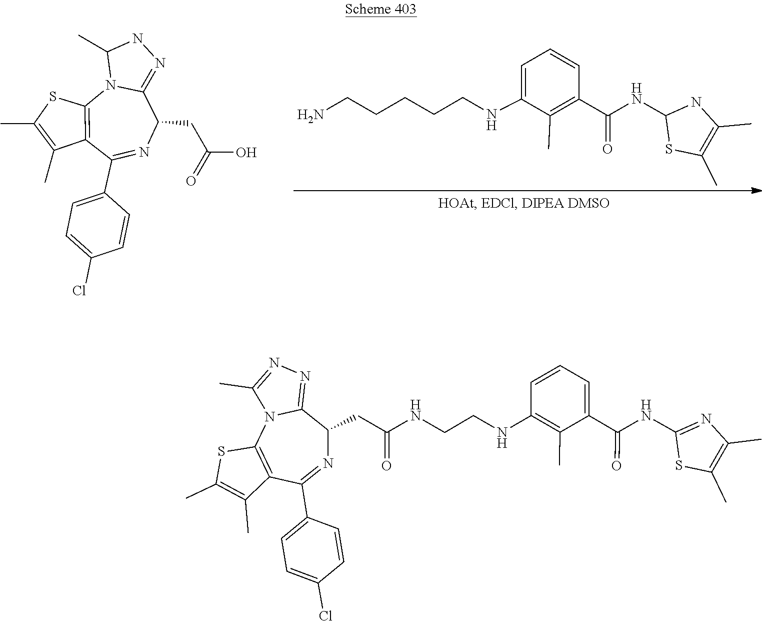

CPD-223 was synthesized following the standard procedure for preparing CPD-220 (2.23 mg, 25% yield). MS (ESI) m/z=729.7 [M+H]⁺.

### Example 406. (S)-3-((6-(2-(4-(4-Chlorophenyl)-2,3,9-trimethyl-6H-thieno[3,2-f][1,2,4]triazolo[4,3-a][1,4]diazepin-6-yl)acetamido)hexyl)amino)-N-(4,5-dimethylthiazol-2-yl)-2-methylbenzamide (CPD-224)

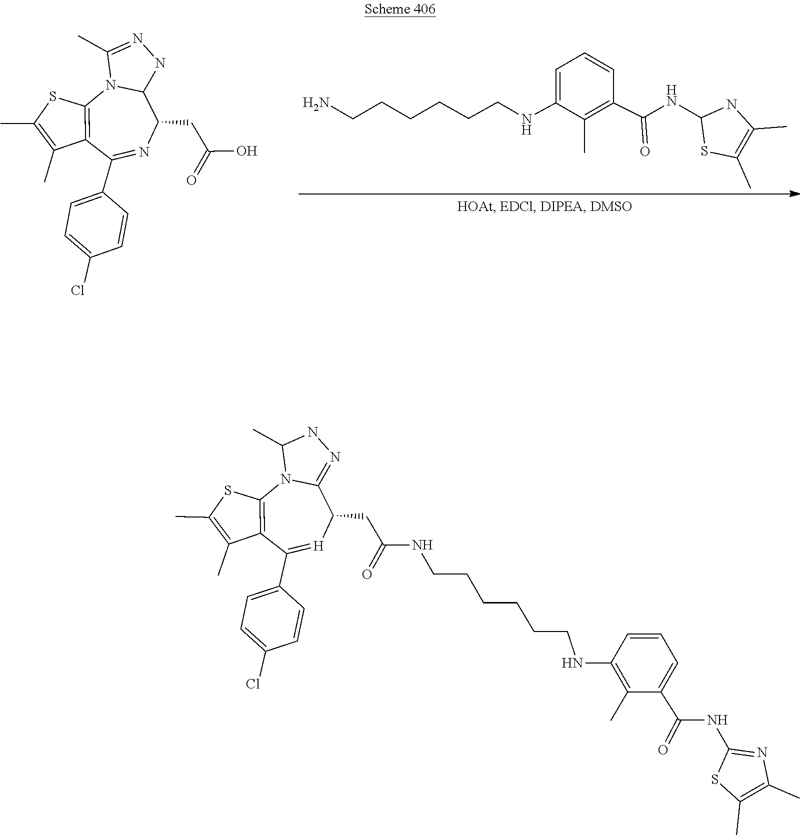

CPD-224 was synthesized following the standard procedure for preparing CPD-220 (2.93 mg, 31% yield). MS (ESI) m/z=743.7 [M+H]⁺.

### Example 407. (S)-3-((7-(2-(4-(4-Chlorophenyl)-2,3,9-trimethyl-6H-thieno[3,2-f][1,2,4]triazolo[4,3-a][1,4]diazepin-6-yl)acetamido)heptyl)amino)-N-(4,5-dimethylthiazol-2-yl)-2-methylbenzamide (CPD-225)

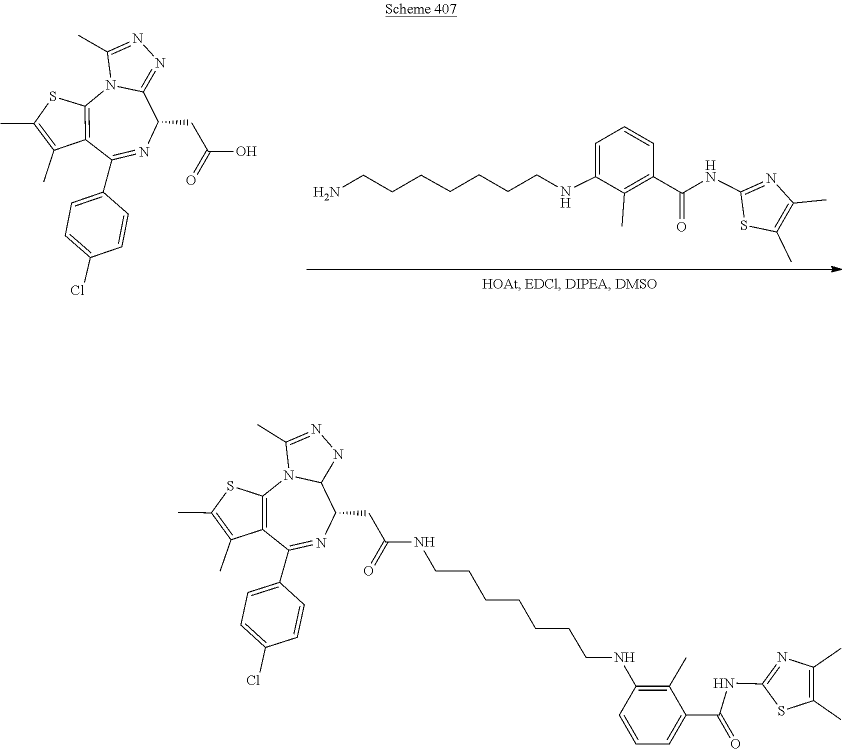

CPD-225 was synthesized following the standard procedure for preparing CPD-220 (3.13 mg, 33% yield). MS (ESI) m/z=757.7 [M+H]⁺.

### Example 408. (S)-3-((8-(2-(4-(4-Chlorophenyl)-2,3,9-trimethyl-6H-thieno[3,2-f][1,2,4]triazolo[4,3-a][1,4]diazepin-6-yl)acetamido)octyl)amino)-N-(4,5-dimethylthiazol-2-yl)-2-methylbenzamide (CPD-226)

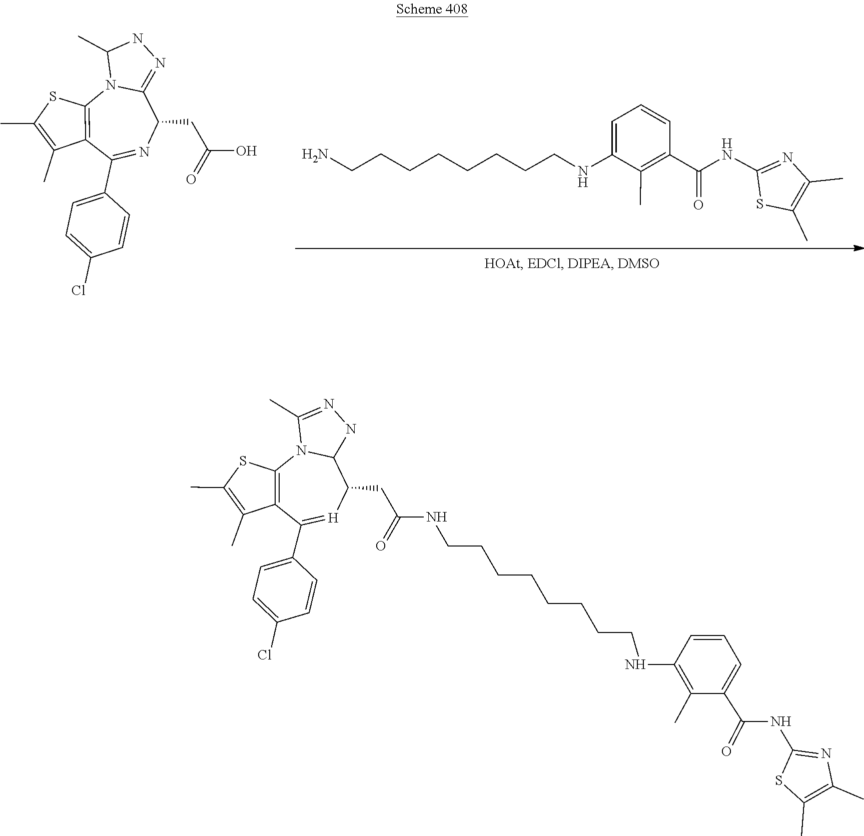

CPD-226 was synthesized following the standard procedure for preparing CPD-220 (2.44 mg, 25% yield). MS (ESI) m/z=771.7 [M+H]⁺.

### Example 409. (S)-3-((2-(2-(2-(4-(4-Chlorophenyl)-2,3,9-trimethyl-6H-thieno[3,2-f][1,2,4]triazolo[4,3-a][1,4]diazepin-6-yl)acetamido)ethoxy)ethyl)amino)-N-(4,5-dimethylthiazol-2-yl)-2-methylbenzamide (CPD-227)

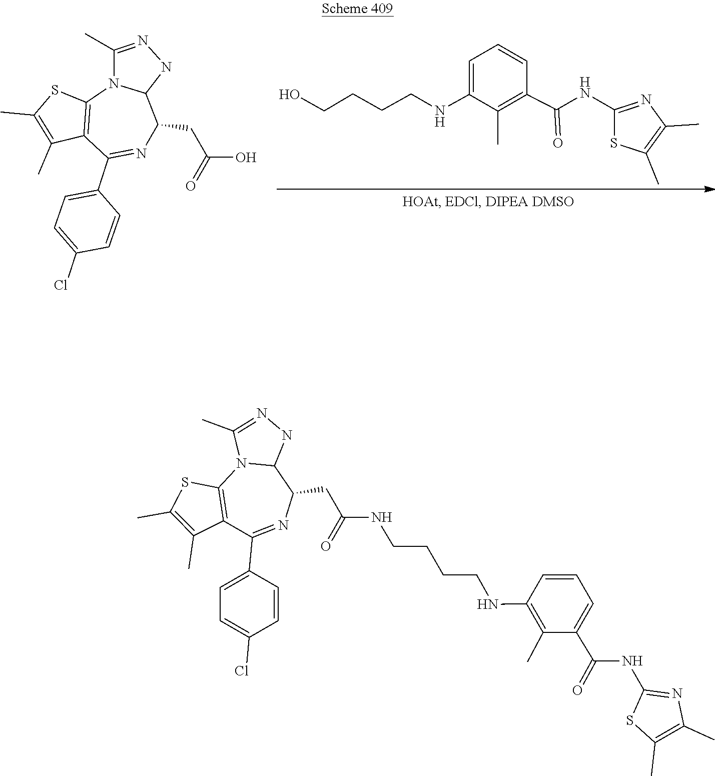

CPD-227 was synthesized following the standard procedure for preparing CPD-220 (2.45 mg, 27% yield). MS (ESI) m/z=731.7 [M+H]⁺.

### Example 410. (S)-3-((2-(2-(2-(2-(4-(4-Chlorophenyl)-2,3,9-trimethyl-6H-thieno[3,2-f][1,2,4]triazolo[4,3-a][1,4]diazepin-6-yl)acetamido)ethoxy)ethoxy)ethyl)amino)-N-(4,5-dimethylthiazol-2-yl)-2-methylbenzamide (CPD-228)

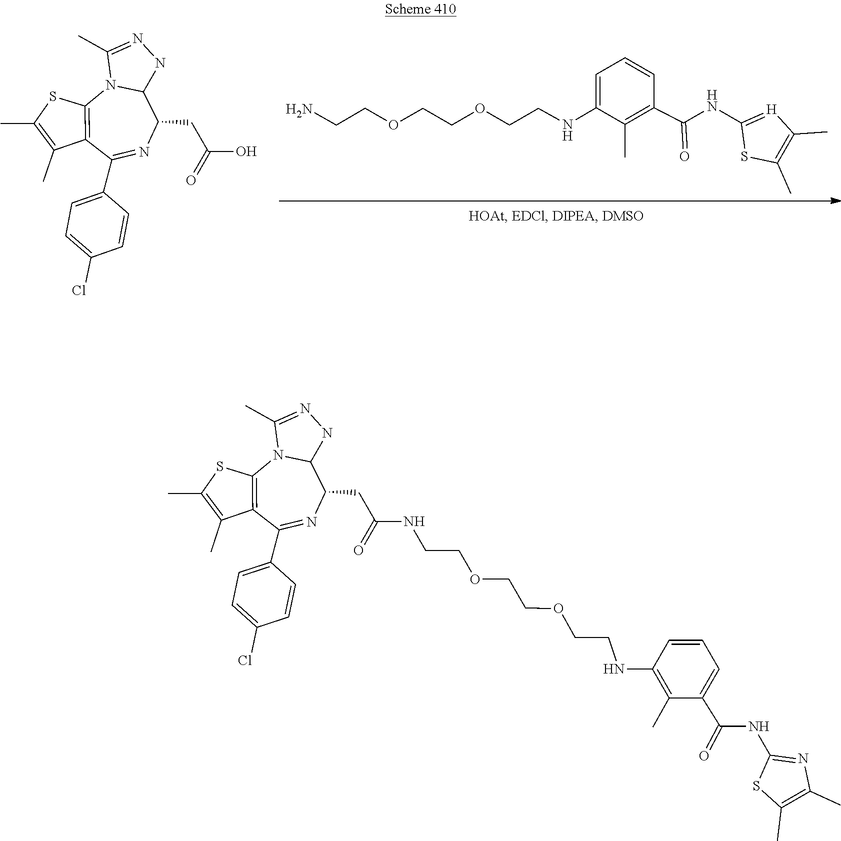

CPD-228 was synthesized following the standard procedure for preparing CPD-220 (2.11 mg, 22% yield). MS (ESI) m/z=775.7 [M+H]⁺.

### Example 411. (S)-3-((1-(4-(4-Chlorophenyl)-2,3,9-trimethyl-6H-thieno[3,2-f][1,2,4]triazolo[4,3-a][1,4]diazepin-6-yl)-2-oxo-6,9,12-trioxa-3-azatetradecan-14-yl)amino)-N-(4,5-dimethylthiazol-2-yl)-2-methylbenzamide (CPD-229)

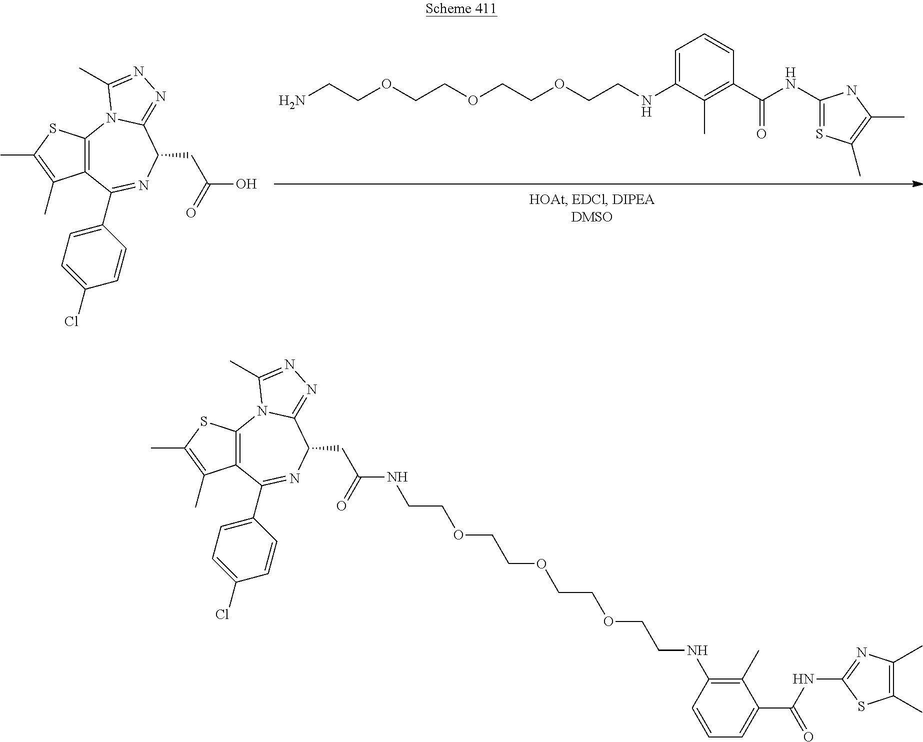

CPD-229 was synthesized following the standard procedure for preparing CPD-220 (2.02 mg, 20% yield). MS (ESI) m/z=819.7 [M+H]⁺.

### Example 412. (S)-3-((1-(4-(4-Chlorophenyl)-2,3,9-trimethyl-6H-thieno[3,2-f][1,2,4]triazolo[4,3-a][1,4]diazepin-6-yl)-2-oxo-6,9,12,15-tetraoxa-3-azaheptadecan-17-yl)amino)-N-(4,5-dimethylthiazol-2-yl)-2-methylbenzamide (CPD-230)

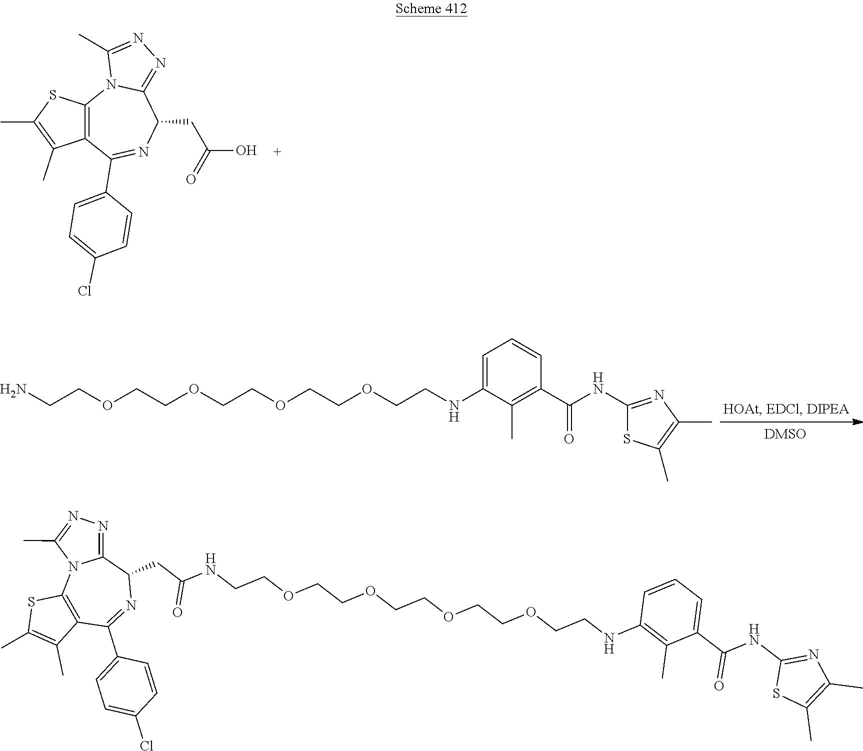

CPD-230 was synthesized following the standard procedure for preparing CPD-220 (3.27 mg, 30% yield). MS (ESI) m/z=863.7 [M+H]⁺.

### Example 413. (S)-3-((1-(4-(4-Chlorophenyl)-2,3,9-trimethyl-6H-thieno[3,2-f][1,2,4]triazolo[4,3-a][1,4]diazepin-6-yl)-2-oxo-6,9,12,15,18-pentaoxa-3-azaicosan-20-yl)amino)-N-(4,5-dimethylthiazol-2-yl)-2-methylbenzamide (CPD-231)

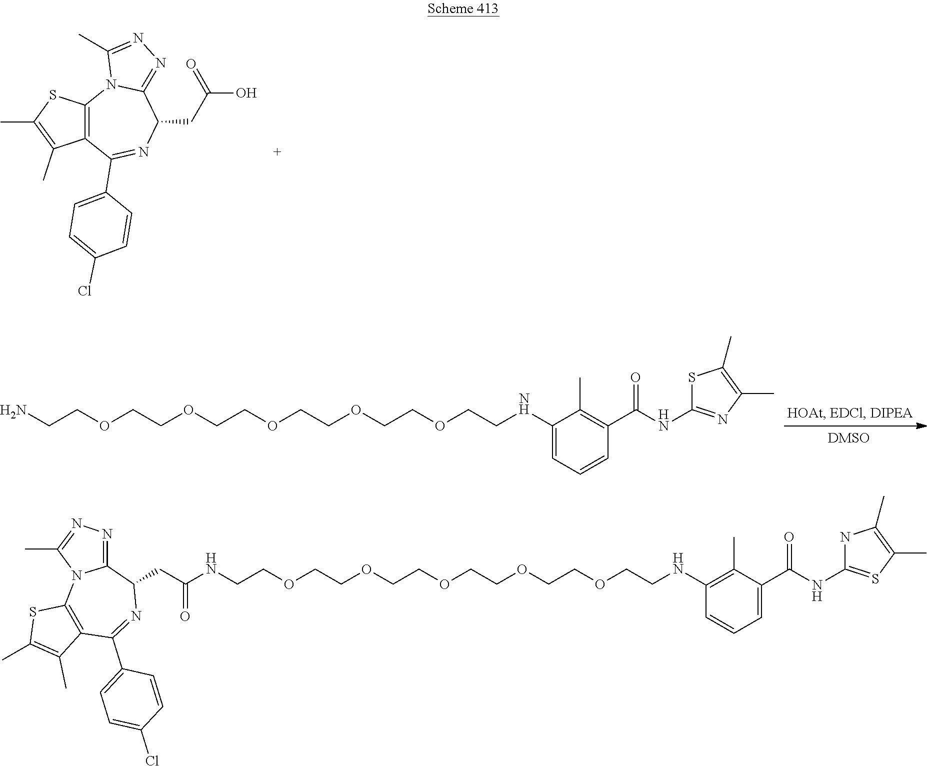

CPD-231 was synthesized following the standard procedure for preparing CPD-220 (1.99 mg, 18% yield). MS (ESI) m/z=907.8 [M+H]⁺.

### Example 414. (S)-2-(3-(2-(4-(4-Chlorophenyl)-2,3,9-trimethyl-6H-thieno[3,2-f][1,2,4]triazolo[4,3-a][1,4]diazepin-6-yl)acetamido)propanamido)-N-(4,5-dimethylthiazol-2-yl)benzamide (CPD-232)

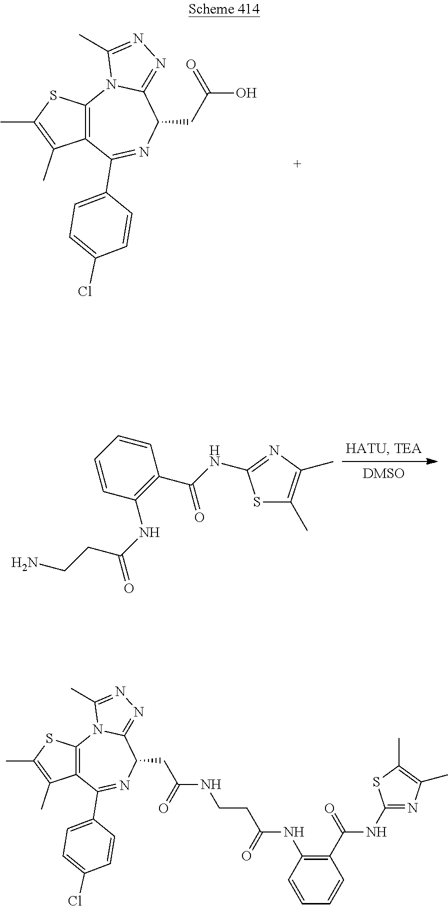

CPD-232 was synthesized following the standard procedure for preparing CPD-219 (5.4 mg, 25% yield). MS (ESI) m/z=701.6 [M+H]⁺.

### Example 415. (S)-2-(4-(2-(4-(4-Chlorophenyl)-2,3,9-trimethyl-6H-thieno[3,2-f][1,2,4]triazolo[4,3-a][1,4]diazepin-6-yl)acetamido)butanamido)-N-(4,5-dimethylthiazol-2-yl)benzamide (CPD-233)

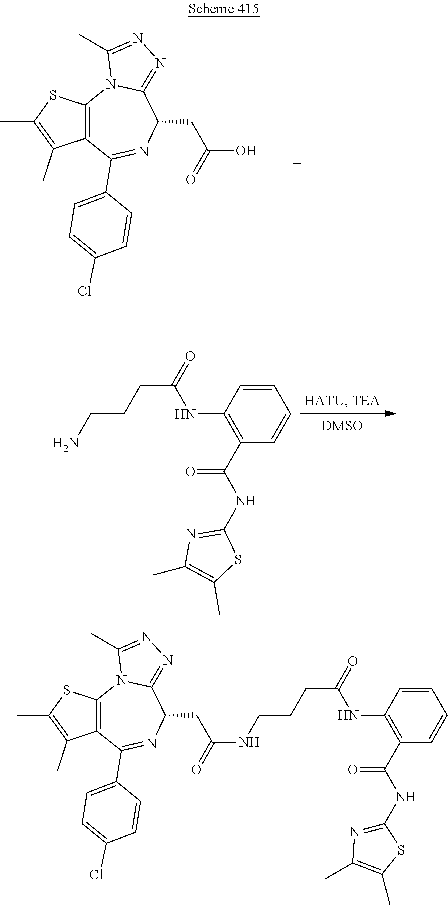 [SMILES: Cc1sc2c(c1C)C(c1ccc(Cl)cc1)=N[C@@H](CC(=O)O)c1nnc(C)n1-2 ; Cc1nc(NC(=O)c2ccccc2NC(=O)CCCN)sc1C ; Cc1nc(NC(=O)c2ccccc2NC(=O)CCCNC(=O)C[C@@H]2N=C(c3ccc(Cl)cc3)c3c(sc(C)c3C)-n3c(C)nnc32)sc1C]

CPD-233 was synthesized following the standard procedure for preparing CPD-219 (7.9 mg, 36% yield). MS (ESI) m/z=715.7 [M+H]⁺.

### Example 416. (S)-2-(6-(2-(4-(4-Chlorophenyl)-2,3,9-trimethyl-6H-thieno[3,2-f][1,2,4]triazolo[4,3-a][1,4]diazepin-6-yl)acetamido)hexanamido)-N-(4,5-dimethylthiazol-2-yl)benzamide (CPD-234)

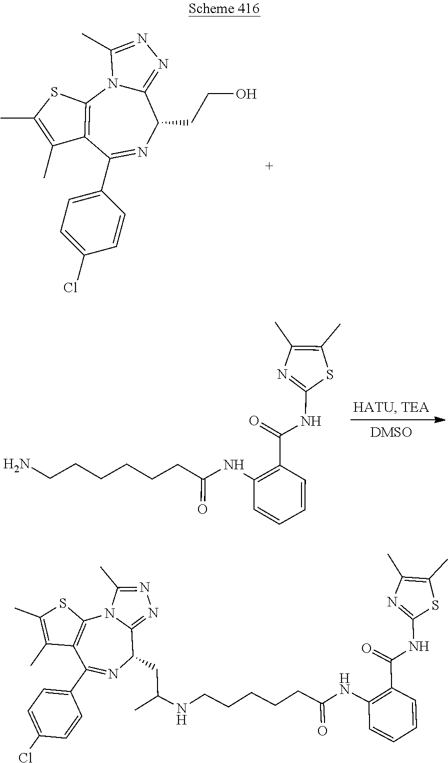

CPD-234 was synthesized following the standard procedure for preparing CPD-219 (3.4 mg, 14% yield). MS (ESI) m/z=743.7 [M+H]⁺.

### Example 417. (S)-2-(7-(2-(4-(4-Chlorophenyl)-2,3,9-trimethyl-6H-thieno[3,2-f][1,2,4]triazolo[4,3-a][1,4]diazepin-6-yl)acetamido)heptanamido)-N-(4,5-dimethylthiazol-2-yl)benzamide (CPD-235)

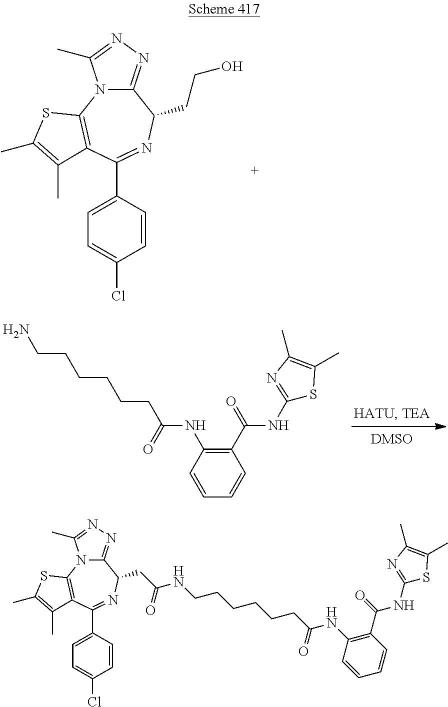

CPD-235 was synthesized following the standard procedure for preparing CPD-219 (2.6 mg, 13% yield). MS (ESI) m/z=757.7 [M+H]⁺.

### Example 418. (S)-2-(3-(2-(2-(4-(4-Chlorophenyl)-2,3,9-trimethyl-6H-thieno[3,2-f][1,2,4]triazolo[4,3-a][1,4]diazepin-6-yl)acetamido)ethoxy)propanamido)-N-(4,5-dimethylthiazol-2-yl)benzamide (CPD-236)

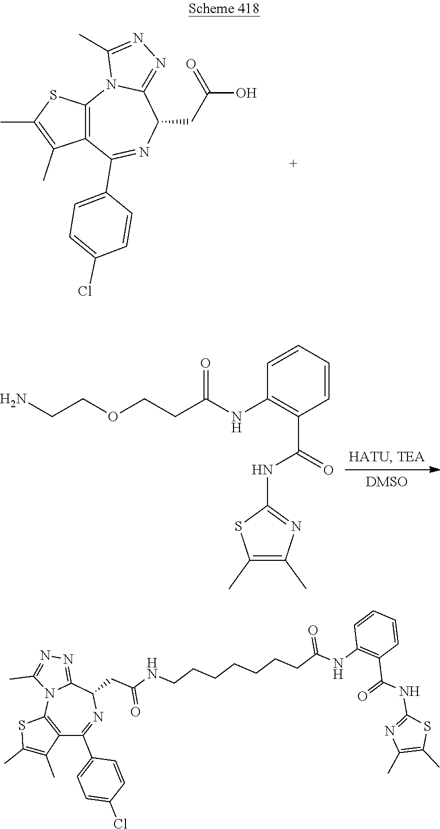

CPD-236 was synthesized following the standard procedure for preparing CPD-219 (8.2 mg, 41% yield). MS (ESI) m/z=745.6 [M+H]⁺.

### Example 419. (S)-2-(3-(2-(2-(2-(4-(4-Chlorophenyl)-2,3,9-trimethyl-6H-thieno[3,2-f][1,2,4]triazolo[4,3-a][1,4]diazepin-6-yl)acetamido)ethoxy)ethoxy)propanamido)-N-(4,5-dimethylthiazol-2-yl)benzamide (CPD-237)

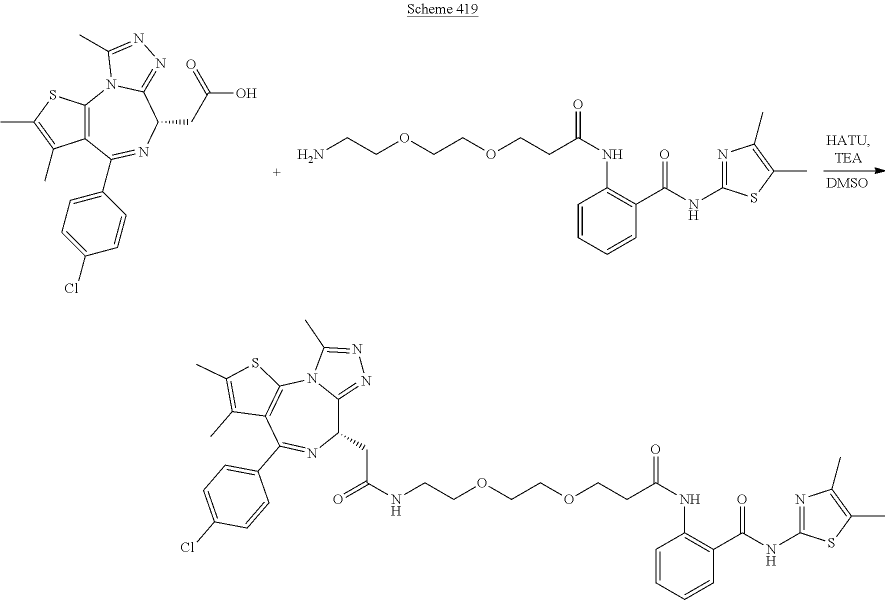

CPD-237 was synthesized following the standard procedure for preparing CPD-219 (4.0 mg, 21% yield). MS (ESI) m/z=789.7 [M+H]⁺.

### Example 420. (S)-2-(1-(4-(4-Chlorophenyl)-2,3,9-trimethyl-6H-thieno[3,2-f][1,2,4]triazolo[4,3-a][1,4]diazepin-6-yl)-2-oxo-6,9,12-trioxa-3-azapentadecan-15-amido)-N-(4,5-dimethylthiazol-2-yl)benzamide (CPD-238)

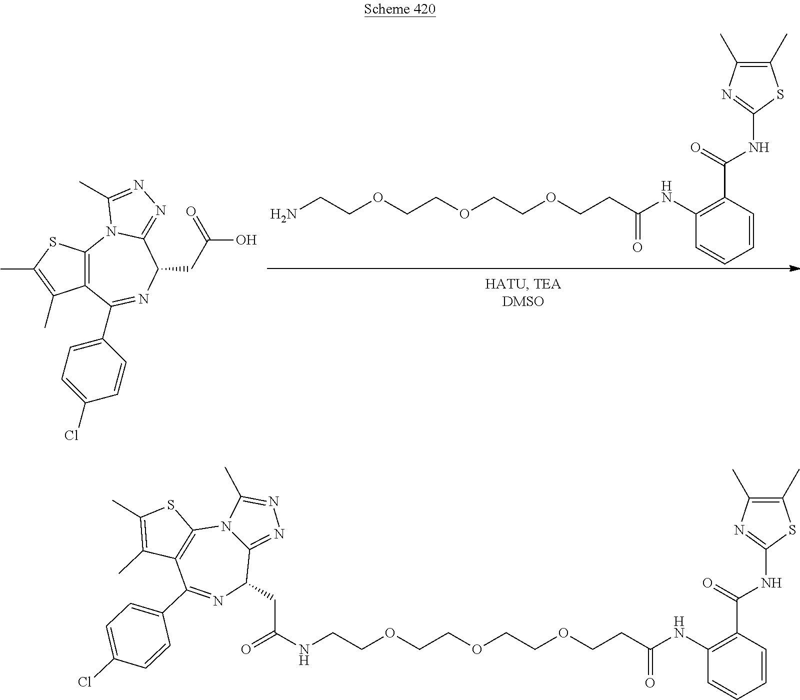

CPD-238 was synthesized following the standard procedure for preparing CPD-219 (5.5 mg, 30% yield). MS (ESI) m/z=833.7 [M+H]⁺.

### Example 421. (S)-2-(1-(4-(4-Chlorophenyl)-2,3,9-trimethyl-6H-thieno[3,2-f][1,2,4]triazolo[4,3-a][1,4]diazepin-6-yl)-2-oxo-6,9,12,15-tetraoxa-3-azaoctadecan-18-amido)-N-(4,5-dimethylthiazol-2-yl)benzamide (CPD-239)

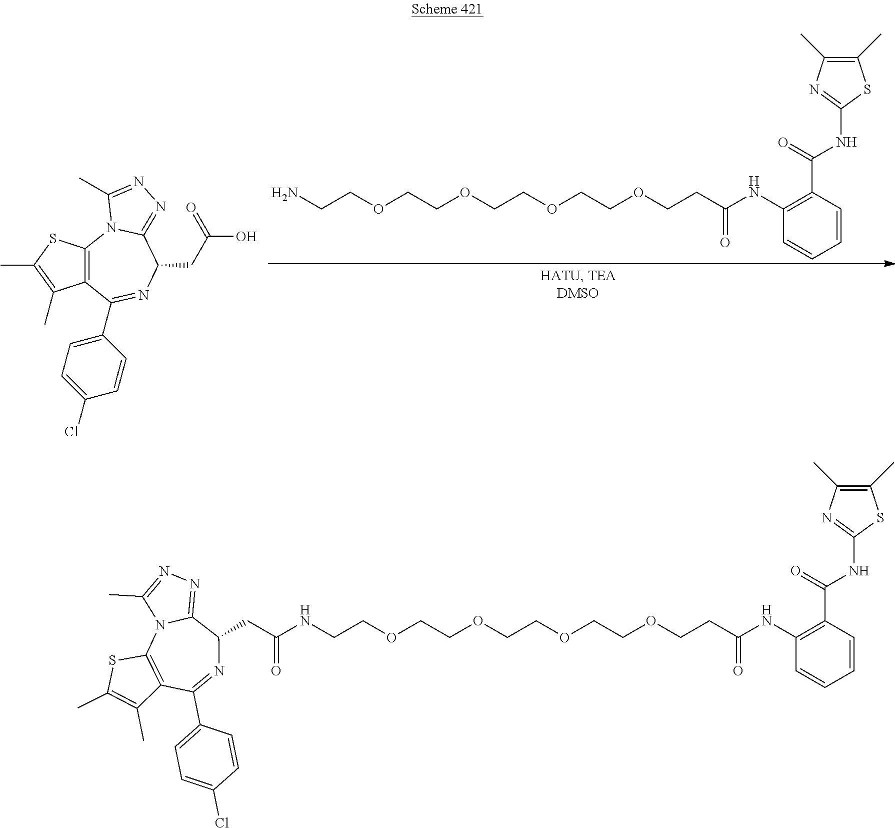

CPD-239 was synthesized following the standard procedure for preparing CPD-219 (6.0 mg, 34% yield). MS (ESI) m/z=877.9 [M+H]⁺.

### Example 422. (S)-2-(1-(4-(4-Chlorophenyl)-2,3,9-trimethyl-6H-thieno[3,2-f][1,2,4]triazolo[4,3-a][1,4]diazepin-6-yl)-2-oxo-6,9,12,15,18-pentaoxa-3-azahenicosan-21-amido)-N-(4,5-dimethylthiazol-2-yl)benzamide (CPD-240)

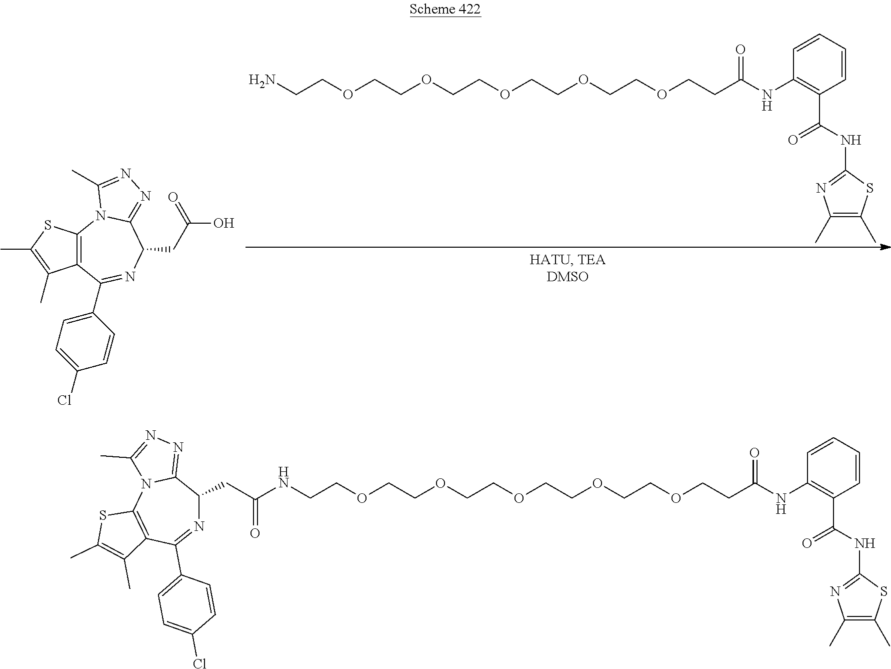

CPD-240 was synthesized following the standard procedure for preparing CPD-219 (6.3 mg, 37% yield). MS (ESI) m/z=922.0 [M+H]⁺.

### Example 423. (S)-2-(1-(4-(4-Chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-amido)-N-(4,5-dimethylthiazol-2-yl)benzamide (CPD-241)

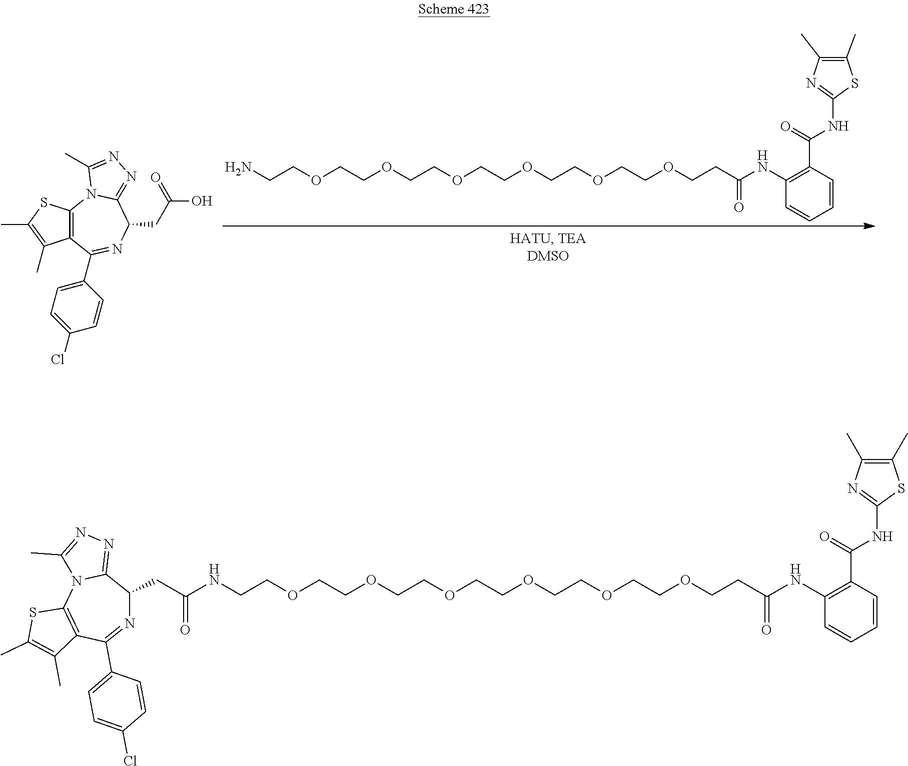

CPD-241 was synthesized following the standard procedure for preparing CPD-219 (3.7 mg, 22% yield). MS (ESI) m/z=965.8 [M+H]⁺.

### Example 424. (S)—N¹-(2-(2-(4-(4-Chlorophenyl)-2,3,9-trimethyl-6H-thieno[3,2-f][1,2,4]triazolo[4,3-a][1,4]diazepin-6-yl)acetamido)ethyl)-N³-(2-((4,5-dimethylthiazol-2-yl)carbamoyl)phenyl)malonamide (CPD-242)

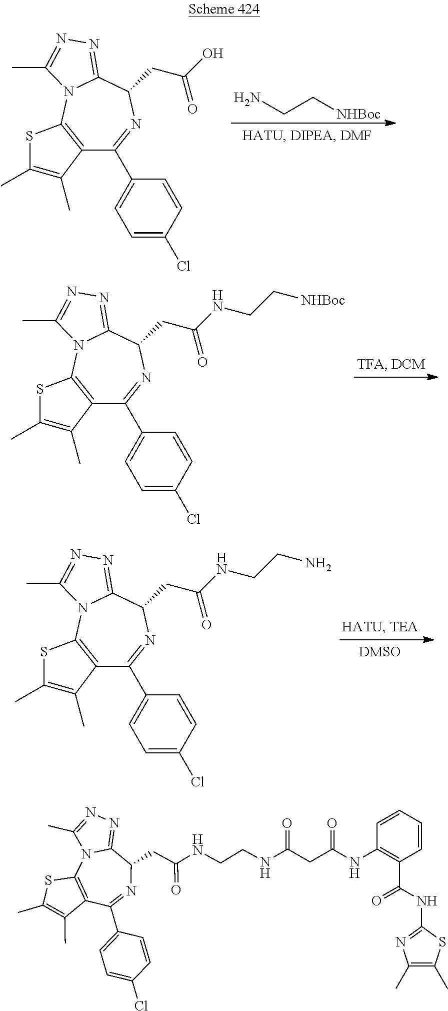

### Step 1. Synthesis of tert-butyl (S)-(2-(2-(4-(4-chlorophenyl)-2,3,9-trimethyl-6H-thieno[3,2-f][1,2,4]triazolo[4,3-a][1,4]diazepin-6-yl)acetamido)ethyl)carbamate

To a solution of (S)-2-(4-(4-chlorophenyl)-2,3,9-trimethyl-6H-thieno[3,2-f][1,2,4]triazolo[4,3-a][1,4]diazepin-6-yl)acetic acid (50 mg, 124 μmol) in DMF (3 mL) were added tert-butyl N-(2-aminoethyl)carbamate (20 mg, 124 μmol), HATU (71 mg, 187 μmol) and DIPEA (48 mg, 374 μmol). After the mixture was stirred at rt for 30 min, it was purified by reverse-phase chromatography (0.1% TFA in water:MeOH=1:1) to provide the title compound (50 mg, 74% yield) as a yellow oil. MS (ESI) m/z=543.2 [M+H]⁺.

### Step 2. Synthesis of (S)—N-(2-aminoethyl)-2-(4-(4-chlorophenyl)-2,3,9-trimethyl-6H-thieno[3,2-f][1,2,4]triazolo[4,3-a][1,4]diazepin-6-yl)acetamide

To a solution of tert-butyl (S)-(2-(2-(4-(4-chlorophenyl)-2,3,9-trimethyl-6H-thieno[3,2-f][1,2,4]triazolo[4,3-a][1,4]diazepin-6-yl)acetamido)ethyl)carbamate (50 mg, 92 μmol) in DCM (2 mL) was added TFA (2 mL). After the mixture was stirred at rt for 30 min, it was purified by reverse-phase chromatography (0.1% TFA in water:MeOH=1:1) to provide the title compound (36 mg, 88% yield) as a yellow solid. MS (ESI) m/z=443.2 [M+H]⁺.

### Step 3. Synthesis of (S)—N¹-(2-(2-(4-(4-chlorophenyl)-2,3,9-trimethyl-6H-thieno[3,2-f][1,2,4]triazolo[4,3-a][1,4]diazepin-6-yl)acetamido)ethyl)-N³-(2-((4,5-dimethylthiazol-2-yl)carbamoyl)phenyl)malonamide

To a mixture of (S)—N-(2-aminoethyl)-2-(4-(4-chlorophenyl)-2,3,9-trimethyl-6H-thieno[3,2-f][1,2,4]triazolo[4,3-a][1,4]diazepin-6-yl)acetamide (16 mg, 36 μmol) in DMSO (0.5 mL) were added HATU (23 mg, 60 μmol) and TEA (9 mg, 90 μmol). The reaction mixture was stirred at rt for 30 min. The solution was purified by reverse-phase chromatography (0-70% MeCN in H₂O) to provide the title compound (3.3 mg, 15% yield) as a white solid. MS (ESI) m/z=758.6 [M+H]⁺.

### Example 425. (S)—N¹-(2-(2-(4-(4-Chlorophenyl)-2,3,9-trimethyl-6H-thieno[3,2-f][1,2,4]triazolo[4,3-a][1,4]diazepin-6-yl)acetamido)ethyl)-N⁴-(2-((4,5-dimethylthiazol-2-yl)carbamoyl)phenyl)succinamide (CPD-243)

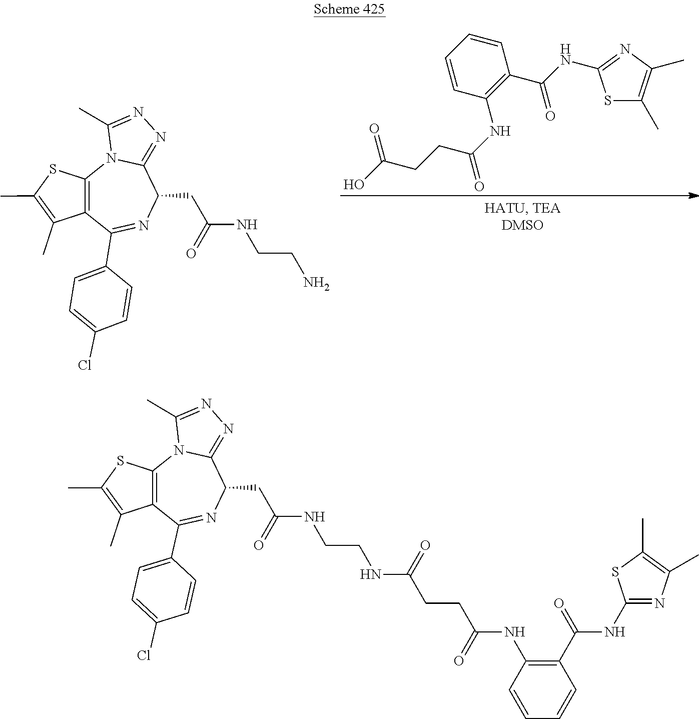

CPD-243 was synthesized following the standard procedure for preparing CPD-242 (2.6 mg, 12% yield). MS (ESI) m/z=772.7 [M+H]⁺.

### Example 426. (S)—N¹-(2-(2-(4-(4-Chlorophenyl)-2,3,9-trimethyl-6H-thieno[3,2-f][1,2,4]triazolo[4,3-a][1,4]diazepin-6-yl)acetamido)ethyl)-N⁵-(2-((4,5-dimethylthiazol-2-yl)carbamoyl)phenyl)glutaramide (CPD-244)

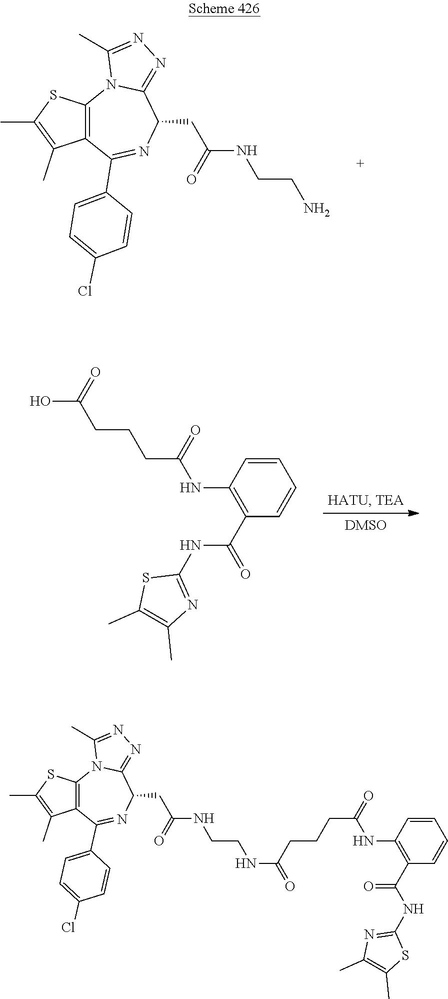 [SMILES: Cc1sc2c(c1C)C(c1ccc(Cl)cc1)=N[C@@H](CC(=O)NCCN)c1nnc(C)n1-2 ; Cc1nc(NC(=O)c2ccccc2NC(=O)CCCC(=O)O)sc1C ; Cc1nc(NC(=O)c2ccccc2NC(=O)CCCC(=O)NCCNC(=O)C[C@@H]2N=C(c3ccc(Cl)cc3)c3c(sc(C)c3C)-n3c(C)nnc32)sc1C]

CPD-244 was synthesized following the standard procedure for preparing CPD-242 (1.6 mg, 7% yield). MS (ESI) m/z=786.7 [M+H]⁺.

### Example 427. (S)—N¹-(2-(2-(4-(4-Chlorophenyl)-2,3,9-trimethyl-6H-thieno[3,2-f][1,2,4]triazolo[4,3-a][1,4]diazepin-6-yl)acetamido)ethyl)-N⁶-(2-((4,5-dimethylthiazol-2-yl)carbamoyl)phenyl)adipamide (CPD-245)

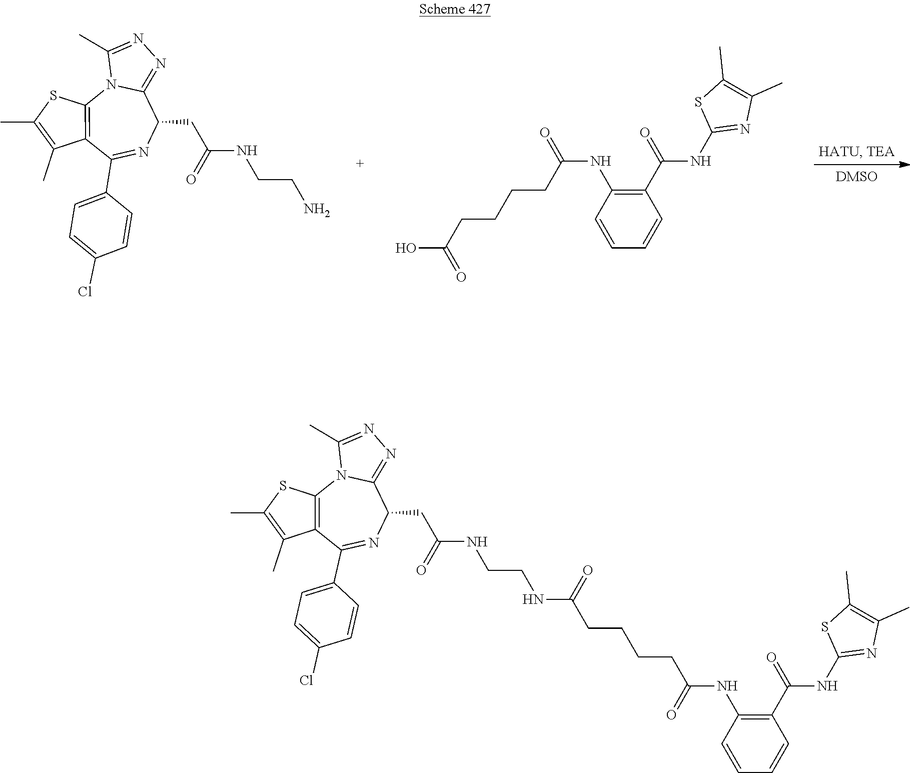

CPD-245 was synthesized following the standard procedure for preparing CPD-242 (2.4 mg, 11% yield). MS (ESI) m/z=800.7 [M+H]⁺.

### Example 428. (S)—N¹-(2-(2-(4-(4-Chlorophenyl)-2,3,9-trimethyl-6H-thieno[3,2-f][1,2,4]triazolo[4,3-a][1,4]diazepin-6-yl)acetamido)ethyl)-N⁷-(2-((4,5-dimethylthiazol-2-yl)carbamoyl)phenyl)heptanediamide (CPD-246)

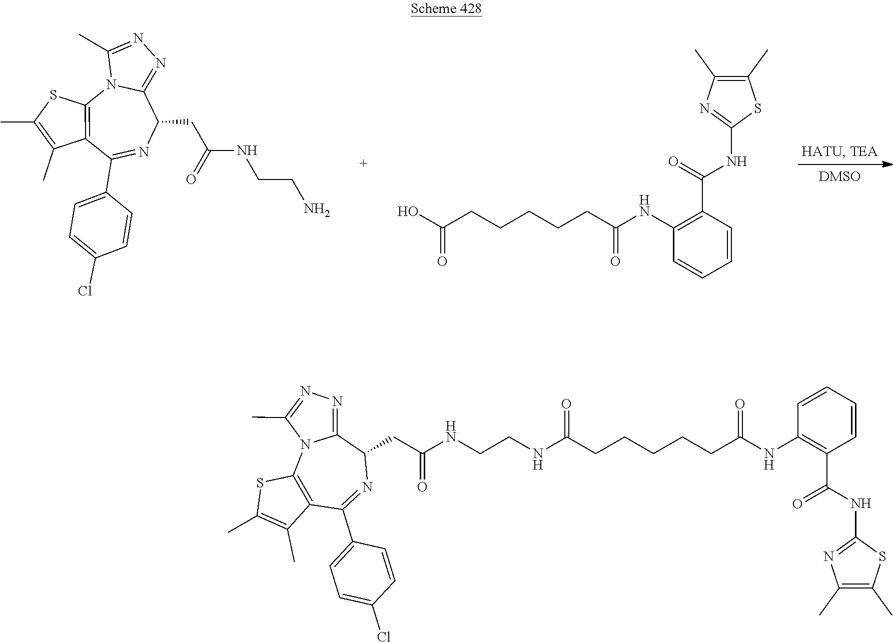

CPD-246 was synthesized following the standard procedure for preparing CPD-242 (2.0 mg, 10% yield). MS (ESI) m/z=814.7 [M+H]⁺.

### Example 429. (S)—N¹-(2-(2-(4-(4-Chlorophenyl)-2,3,9-trimethyl-6H-thieno[3,2-f][1,2,4]triazolo[4,3-a][1,4]diazepin-6-yl)acetamido)ethyl)-N⁸-(2-((4,5-dimethylthiazol-2-yl)carbamoyl)phenyl)octanediamide (CPD-247)

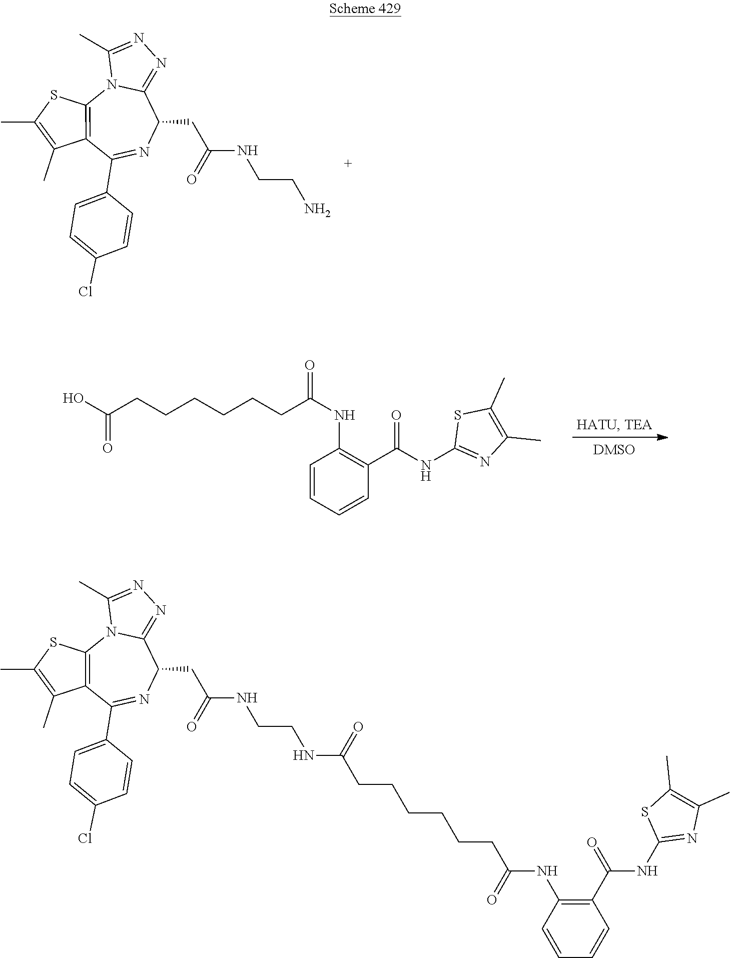

CPD-247 was synthesized following the standard procedure for preparing CPD-242 (2.9 mg, 14% yield). MS (ESI) m/z=828.7 [M+H]⁺.

### Example 430. (S)—N¹-(2-(2-(4-(4-Chlorophenyl)-2,3,9-trimethyl-6H-thieno[3,2-f][1,2,4]triazolo[4,3-a][1,4]diazepin-6-yl)acetamido)ethyl)-N⁹-(2-((4,5-dimethylthiazol-2-yl)carbamoyl)phenyl)nonanediamide (CPD-248)

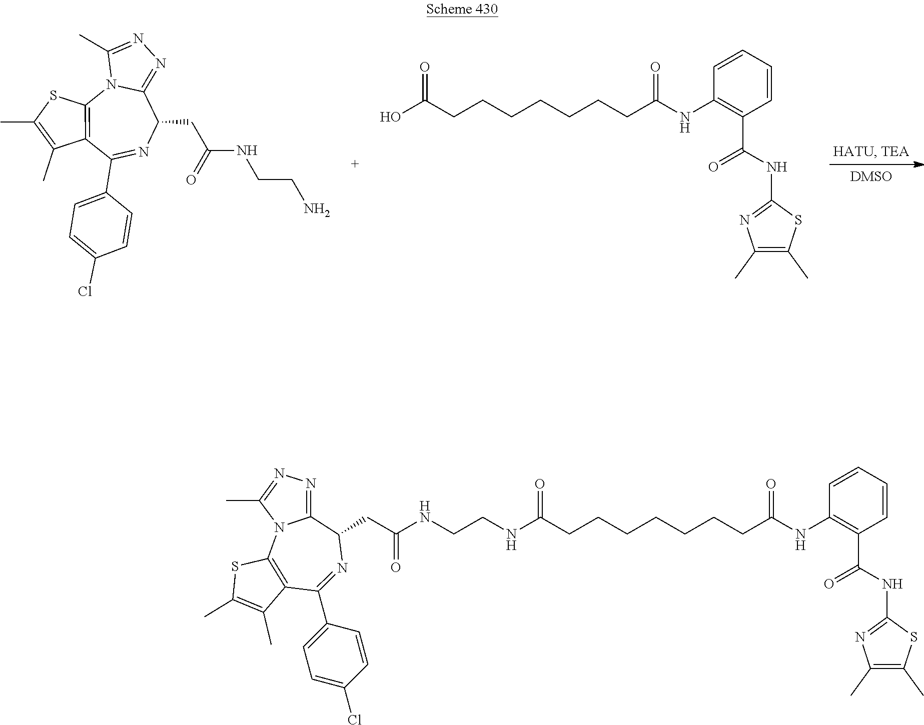

CPD-248 was synthesized following the standard procedure for preparing CPD-242 (3.5 mg, 17% yield). MS (ESI) m/z=842.8 [M+H]⁺.

### Example 431. (S)—N¹-(2-(2-(4-(4-Chlorophenyl)-2,3,9-trimethyl-6H-thieno[3,2-f][1,2,4]triazolo[4,3-a][1,4]diazepin-6-yl)acetamido)ethyl)-N¹⁰-(2-((4,5-dimethylthiazol-2-yl)carbamoyl)phenyl)decanediamide (CPD-249)

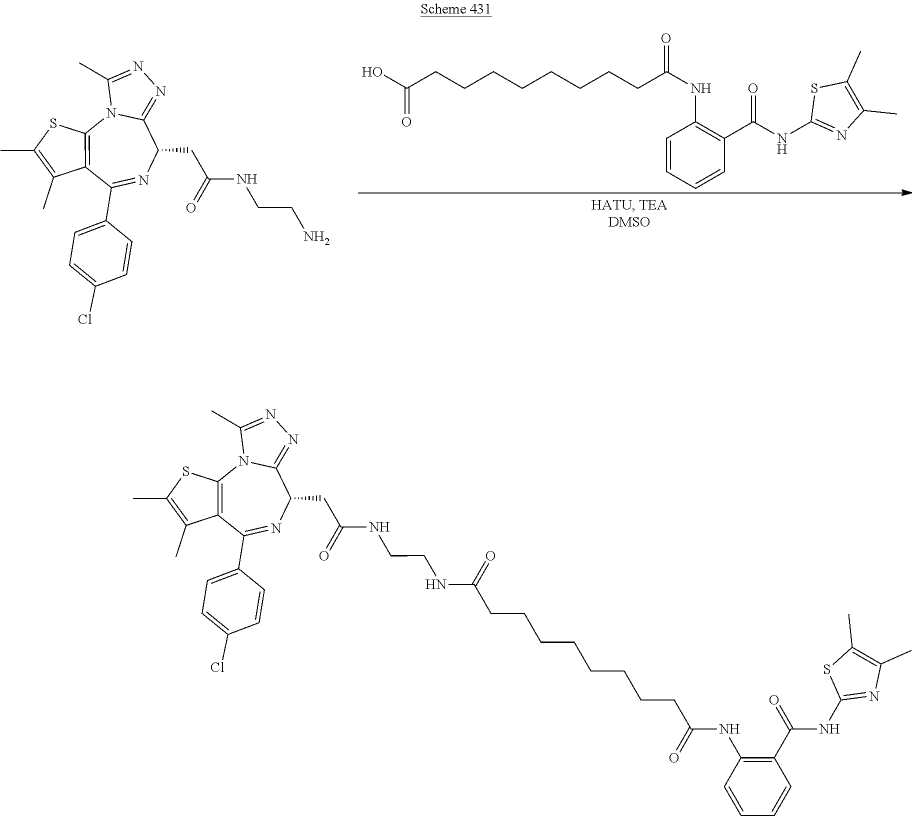

CPD-249 was synthesized following the standard procedure for preparing CPD-242 (3.3 mg, 17% yield). MS (ESI) m/z=856.7 [M+H]⁺.

### Example 432. (S)-2-(1-(4-(4-Chlorophenyl)-2,3,9-trimethyl-6H-thieno[3,2-f][1,2,4]triazolo[4,3-a][1,4]diazepin-6-yl)-2,7-dioxo-10,13-dioxa-3,6-diazahexadecan-16-amido)-N-(4,5-dimethylthiazol-2-yl)benzamide (CPD-250)

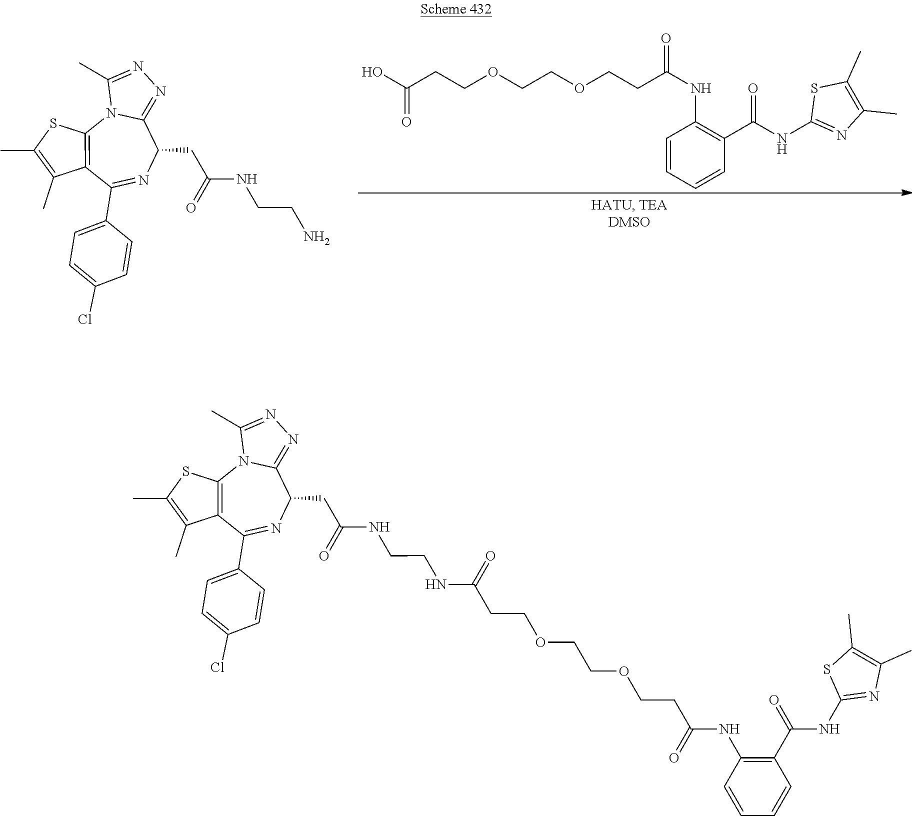

CPD-250 was synthesized following the standard procedure for preparing CPD-242 (3.0 mg, 15% yield). MS (ESI) m/z=860.7 [M+H]⁺.

### Example 433. (S)—N¹-(2-(2-(4-(4-Chlorophenyl)-2,3,9-trimethyl-6H-thieno[3,2-f][1,2,4]triazolo[4,3-a][1,4]diazepin-6-yl)acetamido)ethyl)-N¹⁶-(2-((4,5-dimethylthiazol-2-yl)carbamoyl)phenyl)-4,7,10,13-tetraoxahexadecanediamide (CPD-251)

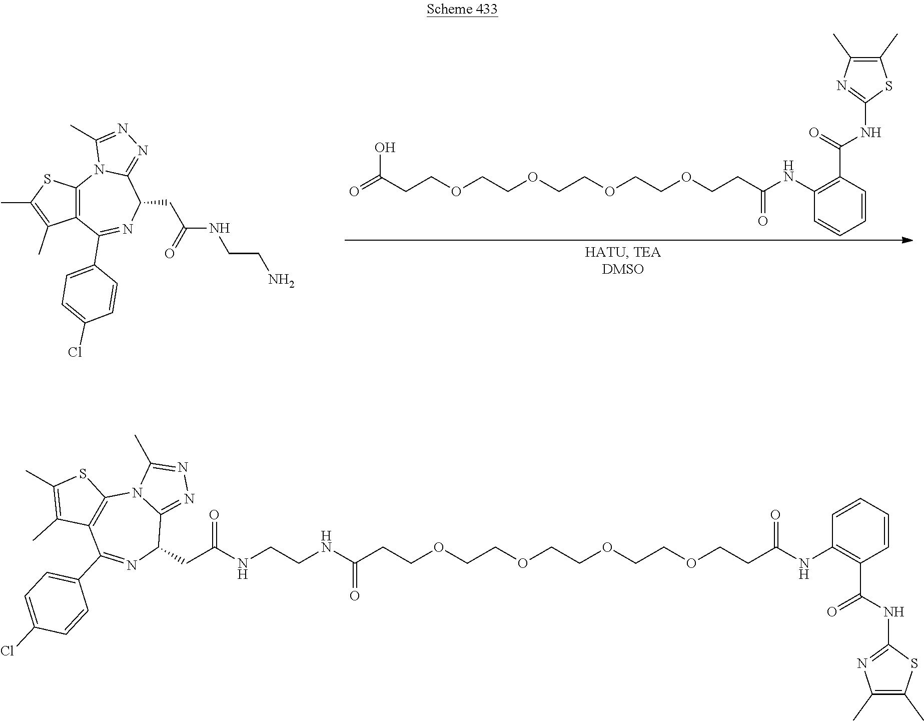 [SMILES: Cc1sc2c(c1C)C(c1ccc(Cl)cc1)=N[C@@H](CC(=O)NCCN)c1nnc(C)n1-2 ; Cc1nc(NC(=O)c2ccccc2NC(=O)CCOCCOCCOCCOCCC(=O)O)sc1C ; Cc1nc(NC(=O)c2ccccc2NC(=O)CCOCCOCCOCCOCCC(=O)NCCNC(=O)C[C@@H]2N=C(c3ccc(Cl)cc3)c3c(sc(C)c3C)-n3c(C)nnc32)sc1C]

CPD-251 was synthesized following the standard procedure for preparing CPD-242 (3.7 mg, 20% yield). MS (ESI) m/z=948.8 [M+H]⁺.

### Example 434. (S)—N¹-(2-(2-(4-(4-Chlorophenyl)-2,3,9-trimethyl-6H-thieno[3,2-f][1,2,4]triazolo[4,3-a][1,4]diazepin-6-yl)acetamido)ethyl)-N¹⁹-(2-((4,5-dimethylthiazol-2-yl)carbamoyl)phenyl)-4,7,10,13,16-pentaoxanonadecanediamide (CPD-252)

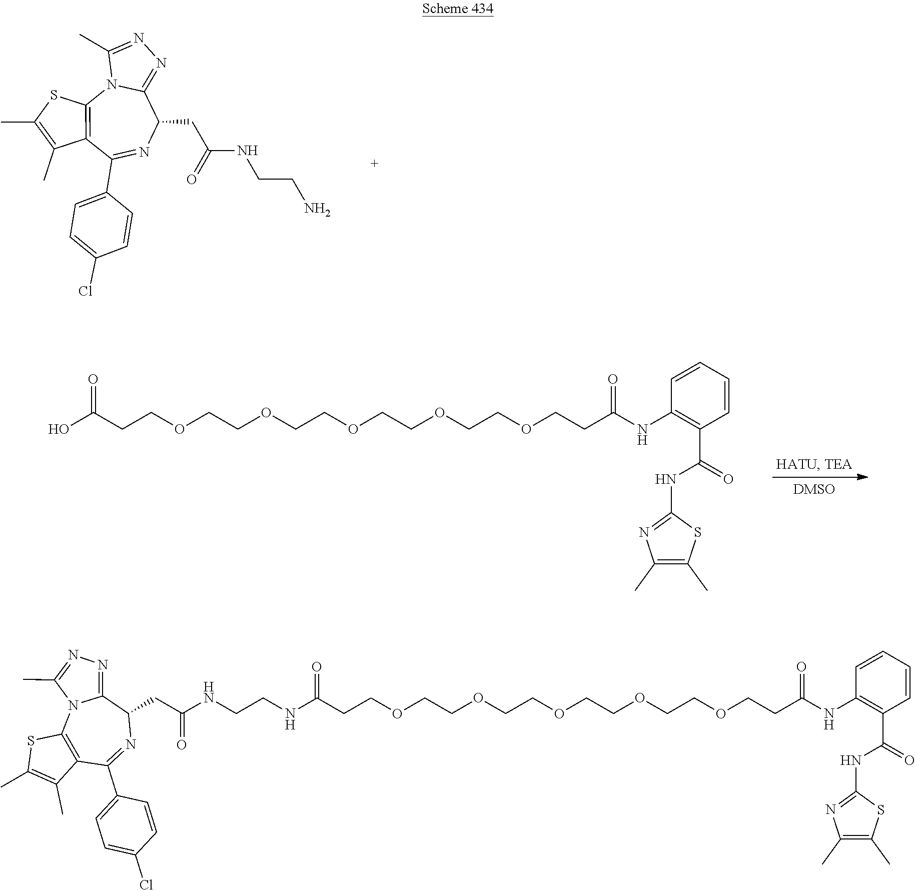 [SMILES: Cc1sc2c(c1C)C(c1ccc(Cl)cc1)=N[C@@H](CC(=O)NCCN)c1nnc(C)n1-2 ; Cc1nc(NC(=O)c2ccccc2NC(=O)CCOCCOCCOCCOCCOCCC(=O)O)sc1C ; Cc1nc(NC(=O)c2ccccc2NC(=O)CCOCCOCCOCCOCCOCCC(=O)NCCNC(=O)C[C@@H]2N=C(c3ccc(Cl)cc3)c3c(sc(C)c3C)-n3c(C)nnc32)sc1C]

CPD-252 was synthesized following the standard procedure for preparing CPD-242 (7.7 mg, 44% yield). MS (ESI) m/z=992.7 [M+H]⁺.

### Example 435. (S)-2-(12-((2-(2-(4-(4-Chlorophenyl)-2,3,9-trimethyl-6H-thieno[3,2-f][1,2,4]triazolo[4,3-a][1,4]diazepin-6-yl)acetamido)ethyl)amino)dodecanamido)-N-(4,5-dimethylthiazol-2-yl)benzamide (CPD-253)

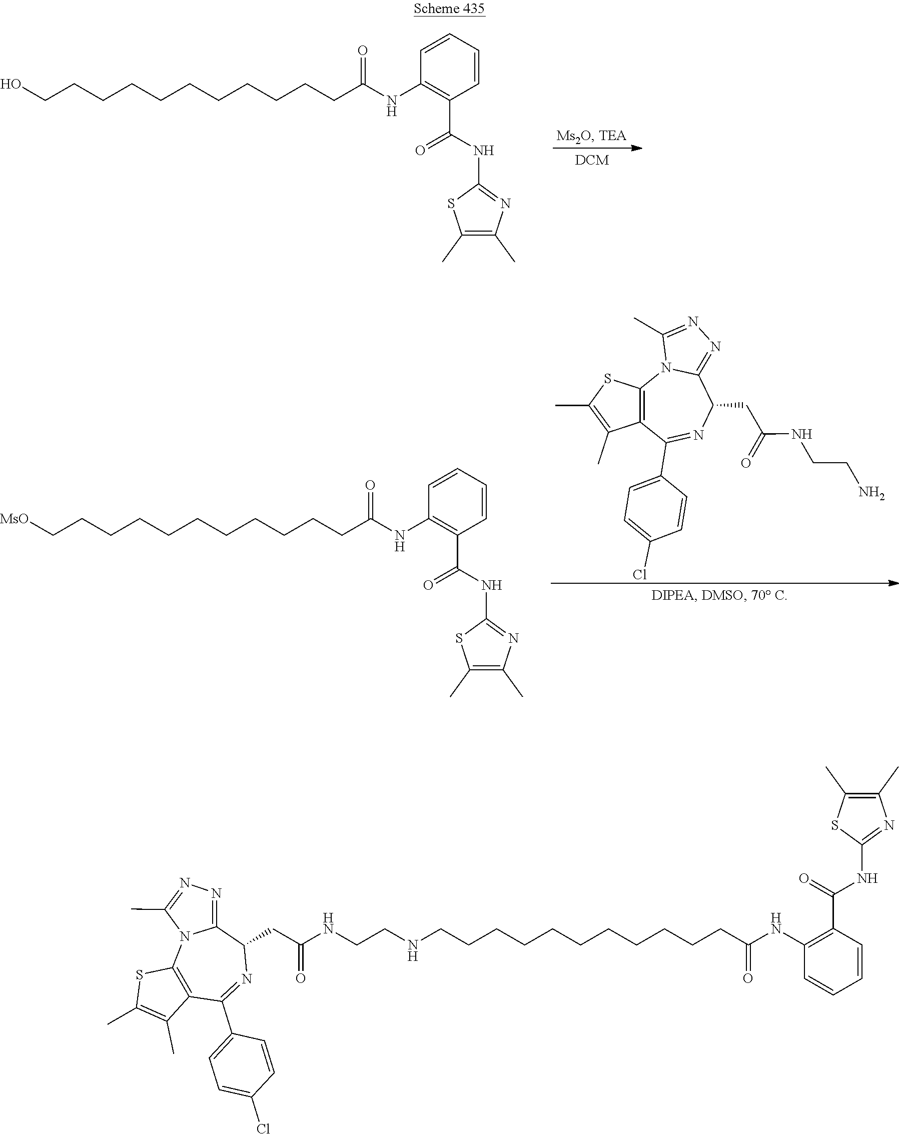

### Step 1. Synthesis of 12-((2-((4,5-dimethylthiazol-2-yl)carbamoyl)phenyl)amino)-12-oxododecyl methanesulfonate

To a stirred mixture of N-(4,5-dimethylthiazol-2-yl)-2-(12-hydroxydodecanoylamino)benzamide (20 mg, 45 μmol) and TEA (14 mg, 134 μmol) in DCM (0.5 mL) was added MsCl (10 mg, 90 μmol). The reaction was stirred at rt for 1 h. The mixture was concentrated and purified by prep-TLC (petroleum ether/EtOAc=1:1) to provide the title compound (20 mg, 85% yield) as a bright oil. MS (ESI) m/z=524.3 [M+H]⁺.

### Step 2. Synthesis of (S)-2-(12-((2-(2-(4-(4-chlorophenyl)-2,3,9-trimethyl-6H-thieno[3,2-f][1,2,4]triazolo[4,3-a][1,4]diazepin-6-yl)acetamido)ethyl)amino)dodecanamido)-N-(4,5-dimethylthiazol-2-yl)benzamide

To a mixture of 12-((2-((4,5-dimethylthiazol-2-yl)carbamoyl)phenyl)amino)-12-oxododecyl methanesulfonate (20 mg, 38 μmol) and (S)—N-(2-aminoethyl)-2-(4-(4-chlorophenyl)-2,3,9-trimethyl-6H-thieno[3,2-f][1,2,4]triazolo[4,3-a][1,4]diazepin-6-yl)acetamide (20 mg, 46 μmol) in DMSO (1 mL) was added DIPEA (15 mg, 114 μmol). The reaction mixture was stirred at 70° C. for 1 h. The solution was purified by reverse-phase chromatography (0-70% MeCN in H₂O) to provide the title compound (5.3 mg, 16% yield) as a yellow solid. MS (ESI) m/z=870.8 [M+H]⁺.

### Example 436. (S)-2-(1-(4-(4-chlorophenyl)-2,3,9-trimethyl-6H-thieno[3,2-f][1,2,4]triazolo[4,3-a][1,4]diazepin-6-yl)-2-oxo-9,12,15-trioxa-3,6-diazaoctadecan-18-amido)-N-(4,5-dimethylthiazol-2-yl)benzamide (CPD-254)

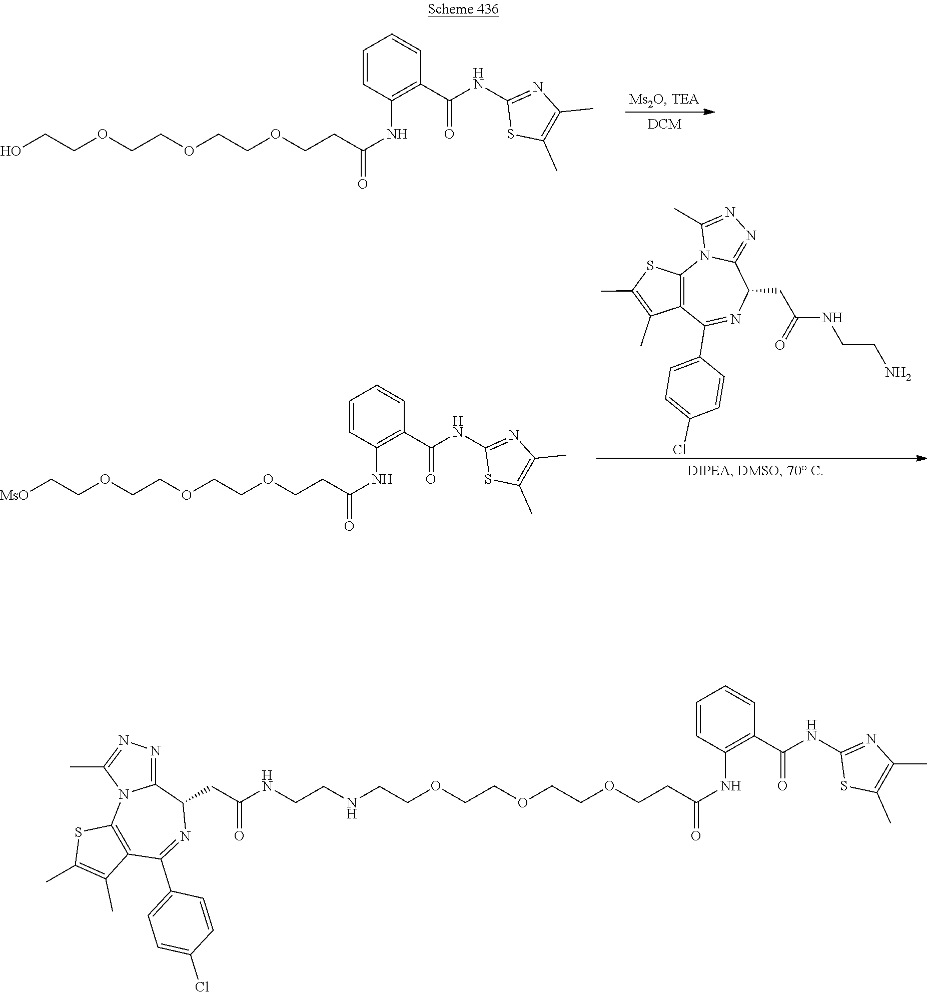

CPD-254 was synthesized following the standard procedure for preparing CPD-253 (5.5 mg, 16% yield). MS (ESI) m/z=856.7 [M+H]⁺.

### Example 437. (S)-2-(2-(2-(4-(4-Chlorophenyl)-2,3,9-trimethyl-6H-thieno[3,2-f][1,2,4]triazolo[4,3-a][1,4]diazepin-6-yl)acetamido)acetamido)-N-(4,5-dimethylthiazol-2-yl)benzamide (CPD-255)

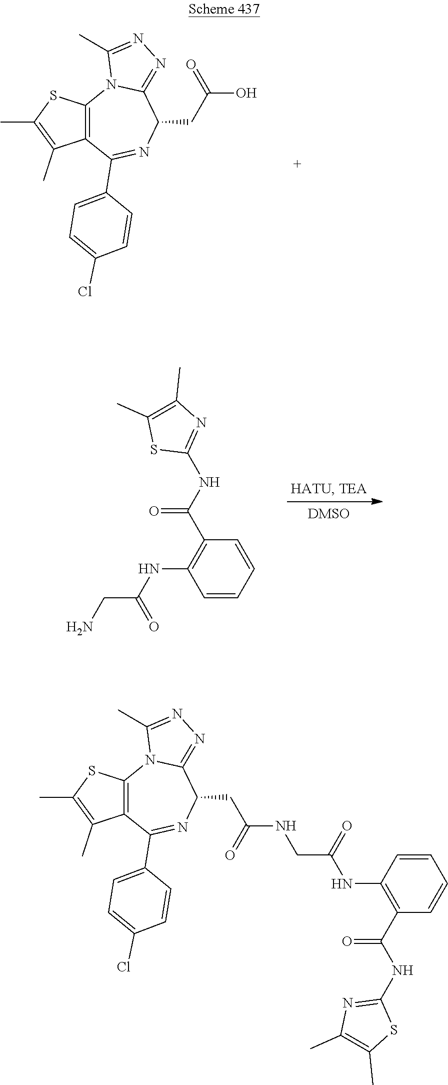 [SMILES: Cc1sc2c(c1C)C(c1ccc(Cl)cc1)=N[C@@H](CC(=O)O)c1nnc(C)n1-2 ; Cc1nc(NC(=O)c2ccccc2NC(=O)CN)sc1C ; Cc1nc(NC(=O)c2ccccc2NC(=O)CNC(=O)C[C@@H]2N=C(c3ccc(Cl)cc3)c3c(sc(C)c3C)-n3c(C)nnc32)sc1C]

CPD-255 was synthesized following the standard procedure for preparing CPD-219 (7.9 mg, 35% yield). MS (ESI) m/z=687.7 [M+H]⁺.

### Example 438. (S)-2-(8-(2-(4-(4-Chlorophenyl)-2,3,9-trimethyl-6H-thieno[3,2-f][1,2,4]triazolo[4,3-a][1,4]diazepin-6-yl)acetamido)octanamido)-N-(4,5-dimethylthiazol-2-yl)benzamide (CPD-256)

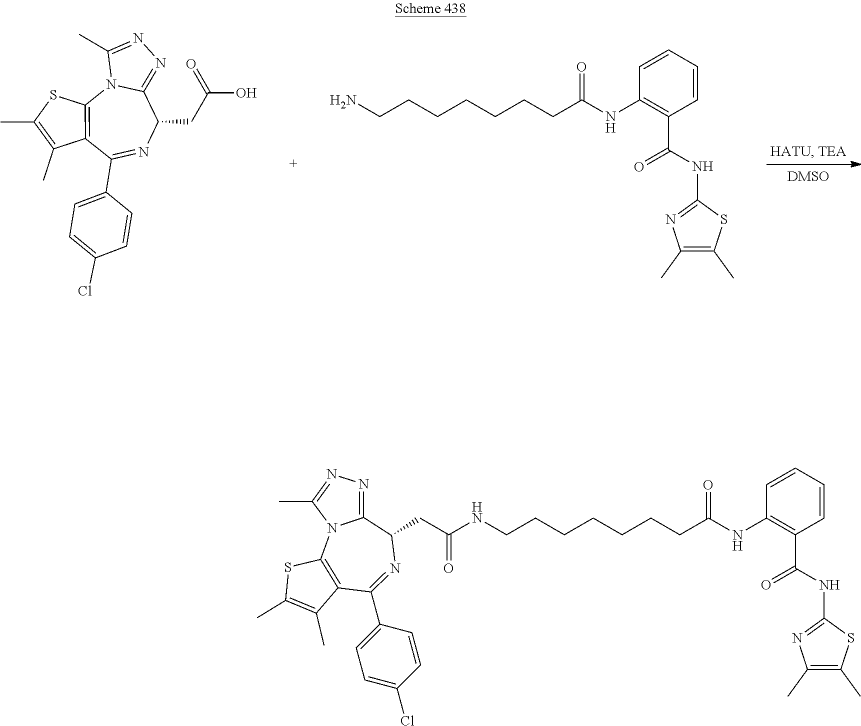

CPD-256 was synthesized following the standard procedure for preparing CPD-219 (5.7 mg, 30% yield). MS (ESI) m/z=771.5 [M+H]⁺.

### Example 439. (S)-2-(3-(3-((2-(2-(4-(4-Chlorophenyl)-2,3,9-trimethyl-6H-thieno[3,2-f][1,2,4]triazolo[4,3-a][1,4]diazepin-6-yl)acetamido)ethyl)amino)-3-oxopropoxy)propanamido)-N-(4,5-dimethylthiazol-2-yl)benzamide (CPD-257)

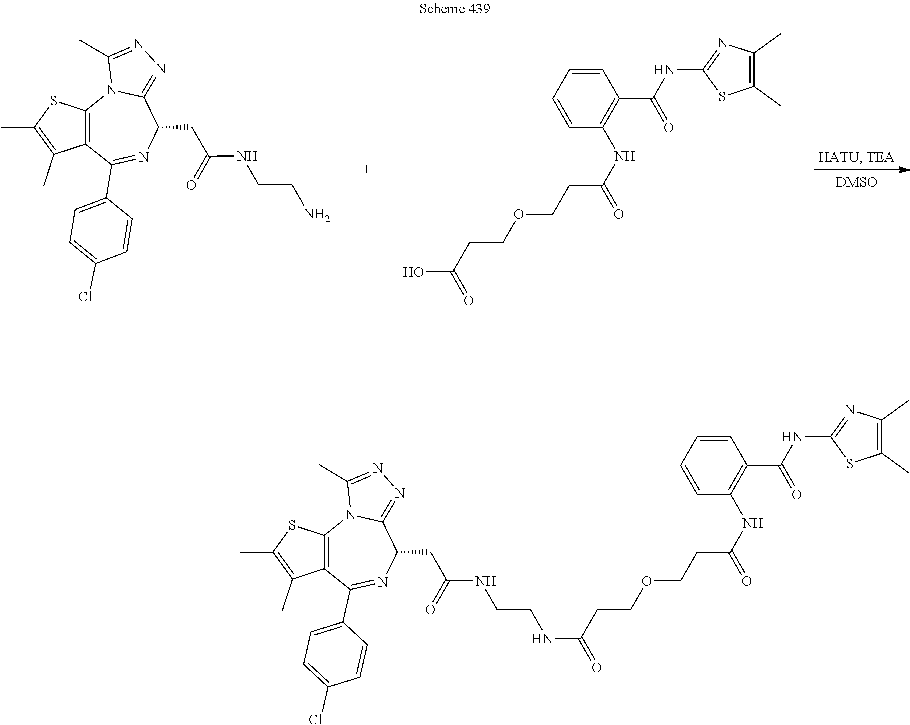

CPD-257 was synthesized following the standard procedure for preparing CPD-242 (5.9 mg, 28% yield). MS (ESI) m/z=816.6 [M+H]⁺.

### Example 440. (S)-2-(9-(2-(4-(4-Chlorophenyl)-2,3,9-trimethyl-6H-thieno[3,2-f][1,2,4]triazolo[4,3-a][1,4]diazepin-6-yl)acetamido)nonanamido)-N-(4,5-dimethylthiazol-2-yl)benzamide (CPD-258)

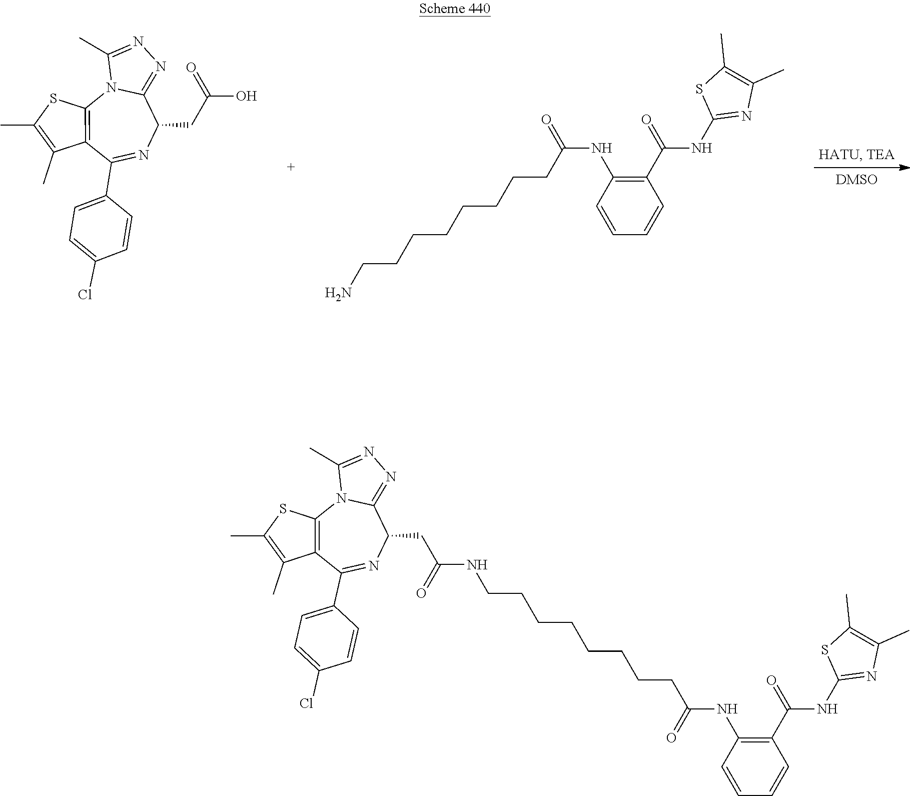

CPD-258 was synthesized following the standard procedure for preparing CPD-219 (2.4 mg, 12% yield). MS (ESI) m/z=785.6[M+H]⁺.

### Example 441. (S)-2-((9-(2-(4-(4-Chlorophenyl)-2,3,9-trimethyl-6H-thieno[3,2-f][1,2,4]triazolo[4,3-a][1,4]diazepin-6-yl)acetamido)nonyl)amino)-N-(4,5-dimethylthiazol-2-yl)benzamide (CPD-259)

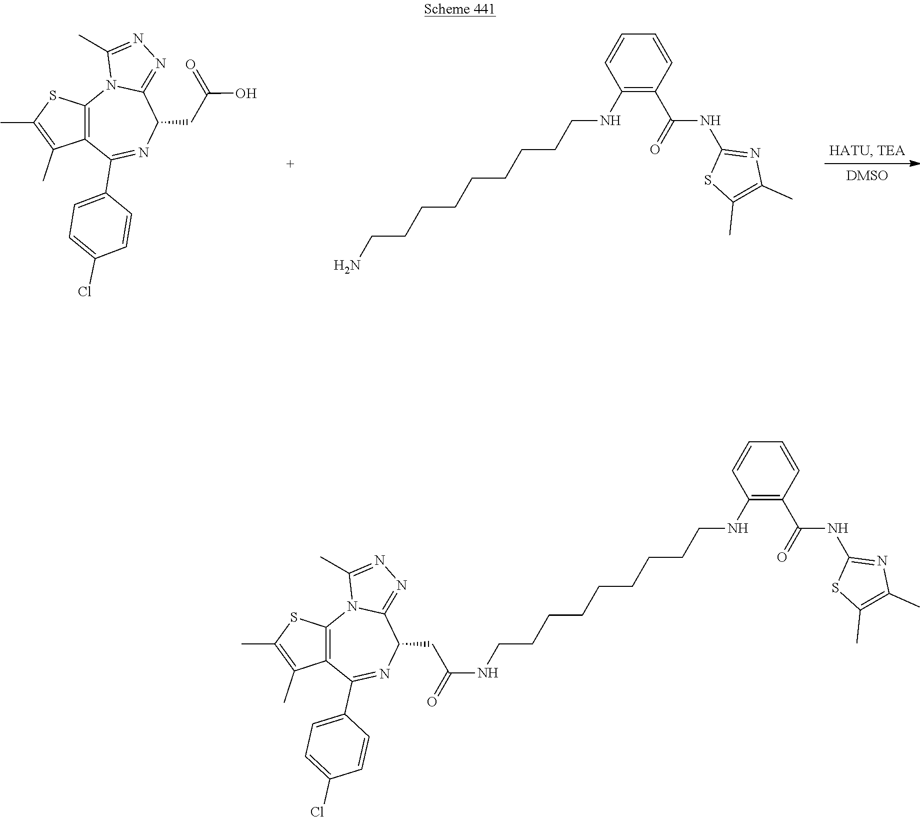

CPD-259 was synthesized following the standard procedure for preparing CPD-219 (1.6 mg, 8% yield). MS (ESI) m/z=771.5 [M+H]⁺.

### Example 442. (S)-2-(1-(4-(4-chlorophenyl)-2,3,9-trimethyl-6H-thieno[3,2-f][1,2,4]triazolo[4,3-a][1,4]diazepin-6-yl)-2,7-dioxo-10,13,16-trioxa-3,6-diazanonadecan-19-amido)-N-(4,5-dimethylthiazol-2-yl)benzamide (CPD-260)

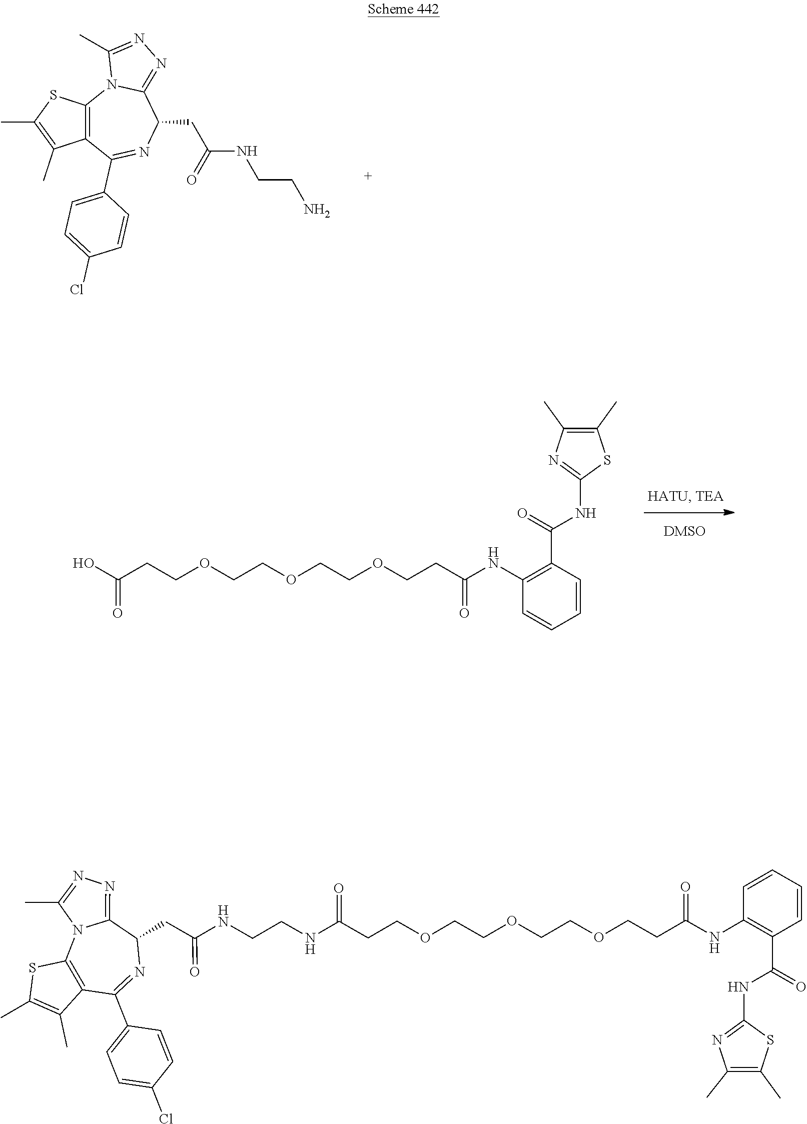

CPD-260 was synthesized following the standard procedure for preparing CPD-242 (2.9 mg, 15% yield). MS (ESI) m/z=904.6 [M+H]⁺.

### Example 443. (S)—N¹-(2-(2-(4-(4-Chlorophenyl)-2,3,9-trimethyl-6H-thieno[3,2-f][1,2,4]triazolo[4,3-a][1,4]diazepin-6-yl)acetamido)ethyl)-N²²-(2-((4,5-dimethylthiazol-2-yl)carbamoyl)phenyl)-4,7,10,13,16,19-hexaoxadocosanediamide (CPD-261)

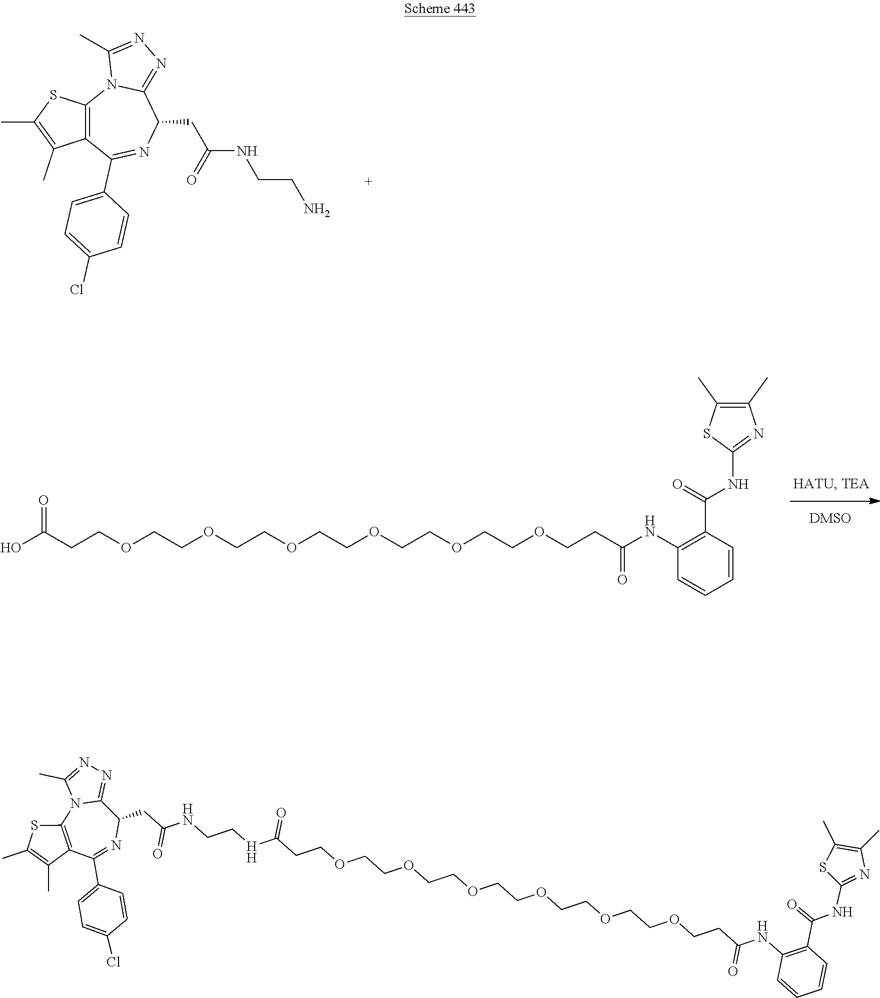

CPD-261 was synthesized following the standard procedure for preparing CPD-242 (7.9 mg, 45% yield). MS (ESI) m/z=1036.6 [M+H]⁺.

### Example 444. (S)-2-((3-(3-(2-(2-(4-(4-Chlorophenyl)-2,3,9-trimethyl-6H-thieno[3,2-f][1,2,4]triazolo[4,3-a][1,4]diazenin-6-yl)acetamido)ethoxy)propoxy)propyl)amino)-N-(4,5-dimethylthiazol-2-yl)benzamide (CPD-262)

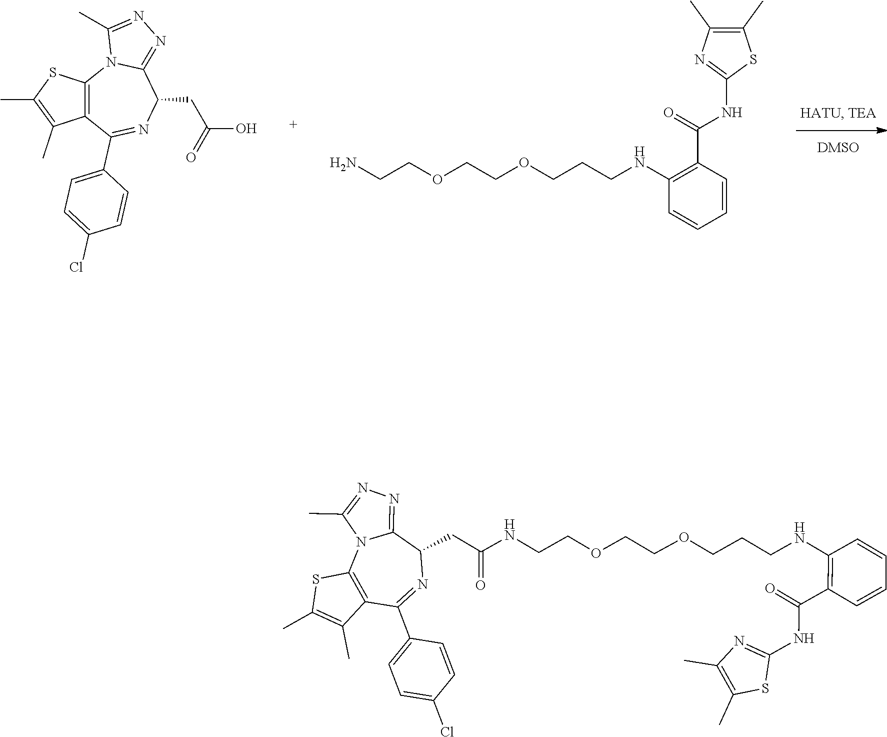 [SMILES: Cc1sc2c(c1C)C(c1ccc(Cl)cc1)=N[C@@H](CC(=O)O)c1nnc(C)n1-2 ; Cc1nc(NC(=O)c2ccccc2NCCCOCCOCCN)sc1C ; Cc1nc(NC(=O)c2ccccc2NCCCOCCOCCNC(=O)C[C@@H]2N=C(c3ccc(Cl)cc3)c3c(sc(C)c3C)-n3c(C)nnc32)sc1C]

CPD-262 was synthesized following the standard procedure for preparing CPD-219 (1.1 mg, 4 yield: 6%). MS (ESI) m/z=775.8 [M+H].

### Example 445. (S)-2-Acetamido-4-((3-(2-(4-(4-chlorophenyl)-2,3,9-trimethyl-6H-thieno[3,2-f][1,2,4]triazolo[4,3-a][1,4]diazepin-6-yl)acetamido)propyl)amino)-N-(4-methyl-5-nitrothiazol-2-yl)benzamide (CPD-263)

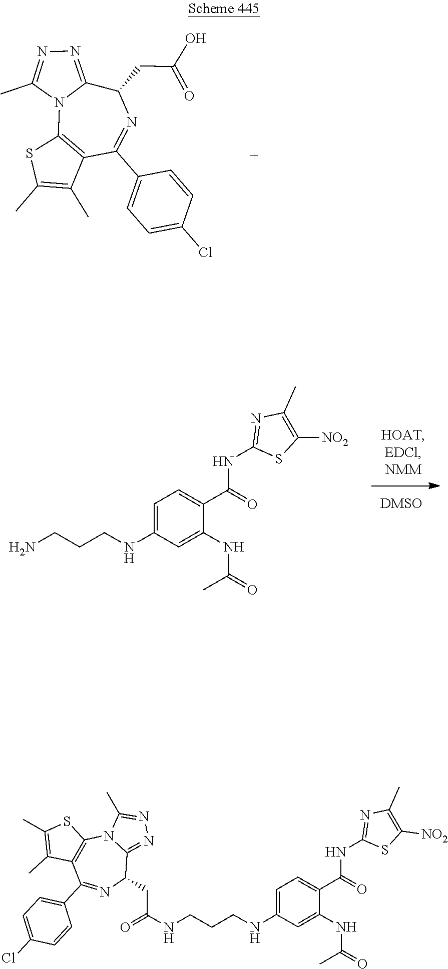 [SMILES: Cc1sc2c(c1C)C(c1ccc(Cl)cc1)=N[C@@H](CC(=O)O)c1nnc(C)n1-2 ; CC(=O)Nc1cc(NCCCN)ccc1C(=O)Nc1nc(C)c([N+](=O)[O-])s1 ; CC(=O)Nc1cc(NCCCNC(=O)C[C@@H]2N=C(c3ccc(Cl)cc3)c3c(sc(C)c3C)-n3c(C)nnc32)ccc1C(=O)Nc1nc(C)c([N+](=O)[O-])s1]

To a mixture of (S)-2-(4-(4-chlorophenyl)-2,3,9-trimethyl-6H-thieno[3,2-f][1,2,4]triazolo[4,3-a][1,4]diazepin-6-yl)acetic acid (4.0 mg, 9.9 μmol) and HOAt (2.01 mg, 14.8 μmol), EDCI (2.82 mg, 14.8 μmol) in DMSO (0.2 mL) were added NMM (2.99 mg, 29.7 μmol) and 2-acetamido-4-((3-aminopropyl)amino)-N-(4-methyl-5-nitrothiazol-2-yl)benzamide (3.92 mg, 9.9 μmol). After the mixture was stirred at 25° C. for 16 h, it was purified by prep-HPLC to provide the title compound (4.96 mg, 65% yield) as a white solid. MS (ESI) m/z=775.5[M+H]⁺.

### Example 446. (S)-2-Acetamido-4-((5-(2-(4-(4-chlorophenyl)-2,3,9-trimethyl-6H-thieno[3,2-f][1,2,4]triazolo[4,3-a][1,4]diazepin-6-yl)acetamido)pentyl)amino)-N-(4-methyl-5-nitrothiazol-2-yl)benzamide (CPD-264)

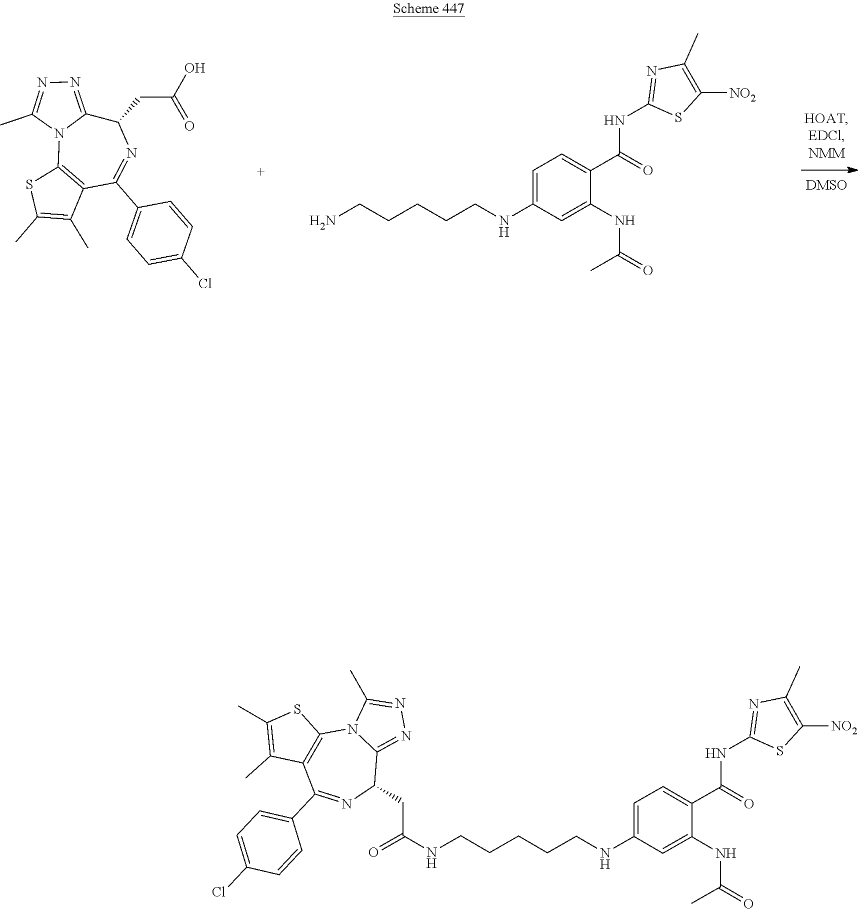 [SMILES: Cc1sc2c(c1C)C(c1ccc(Cl)cc1)=N[C@@H](CC(=O)O)c1nnc(C)n1-2 ; CC(=O)Nc1cc(NCCCCCN)ccc1C(=O)Nc1nc(C)c([N+](=O)[O-])s1 ; CC(=O)Nc1cc(NCCCCCNC(=O)C[C@@H]2N=C(c3ccc(Cl)cc3)c3c(sc(C)c3C)-n3c(C)nnc32)ccc1C(=O)Nc1nc(C)c([N+](=O)[O-])s1]

CPD-264 was synthesized following the standard procedure for preparing CPD-263 (4.19 mg, 53% yield). MS (ESI) m/z=803.6 [M+H]⁺.

### Example 447. (S)-2-Acetamido-4-((9-(2-(4-(4-chlorophenyl)-2,3,9-trimethyl-6H-thieno[3,2-f][1,2,4]triazolo[4,3-a][1,4]diazepin-6-yl)acetamido)nonyl)amino)-N-(4-methyl-5-nitrothiazol-2-yl)benzamide (CPD-265)

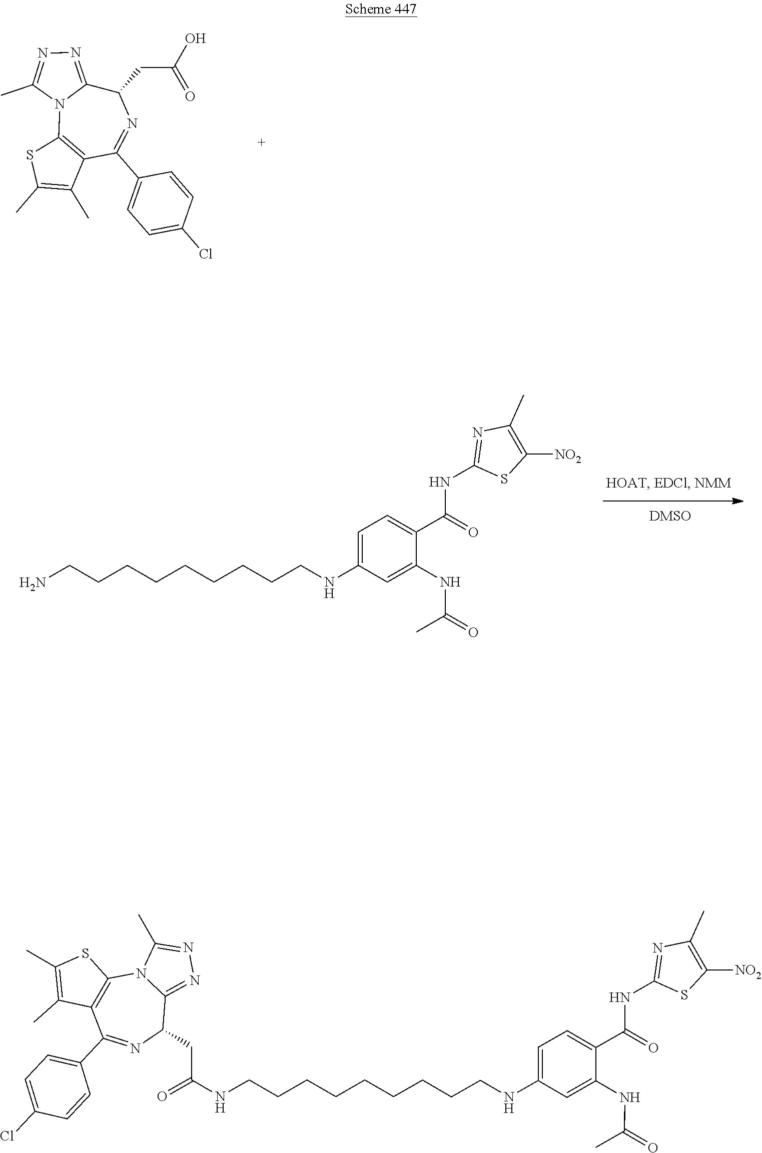 [SMILES: Cc1sc2c(c1C)C(c1ccc(Cl)cc1)=N[C@@H](CC(=O)O)c1nnc(C)n1-2 ; CC(=O)Nc1cc(NCCCCCCCCCN)ccc1C(=O)Nc1nc(C)c([N+](=O)[O-])s1 ; CC(=O)Nc1cc(NCCCCCCCCCNC(=O)C[C@@H]2N=C(c3ccc(Cl)cc3)c3c(sc(C)c3C)-n3c(C)nnc32)ccc1C(=O)Nc1nc(C)c([N+](=O)[O-])s1]

CPD-265 was synthesized following the standard procedure for preparing CPD-263 (5.37 mg, 63% yield). MS (ESI) m/z=859.6 [M+H]⁺.

### Example 448. (S)-2-Acetamido-4-((11-(2-(4-(4-chlorophenyl)-2,3,9-trimethyl-6H-thieno[3,2-f][1,2,4]triazolo[4,3-a][1,4]diazepin-6-yl)acetamido)undecyl)amino)-N-(4-methyl-5-nitrothiazol-2-yl)benzamide (CPD-266)

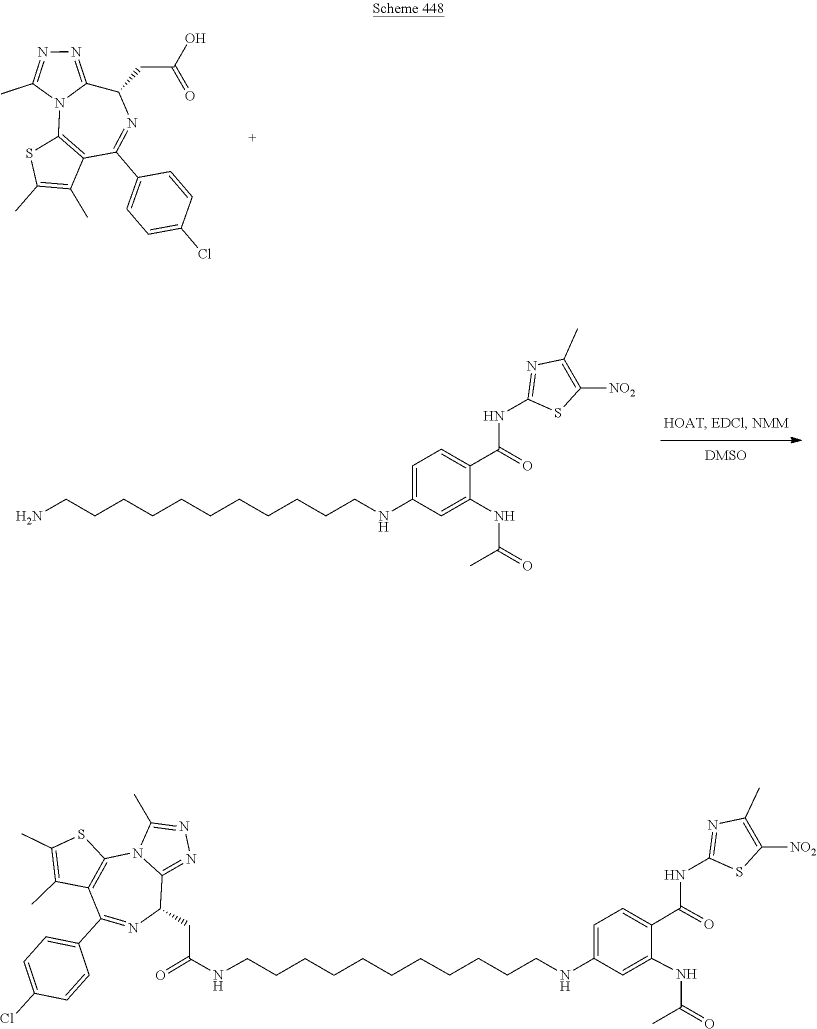

CPD-266 was synthesized following the standard procedure for preparing CPD-263 (5.03 mg, 57% yield). MS (ESI) m/z=887.6 [M+H]⁺.

### Example 449. N-(4,5-dimethylthiazol-2-yl)-2-(3-(2-(2-(2-hydroxyethoxy)ethoxy)ethoxy)propanamido)benzamide (BL1-173)

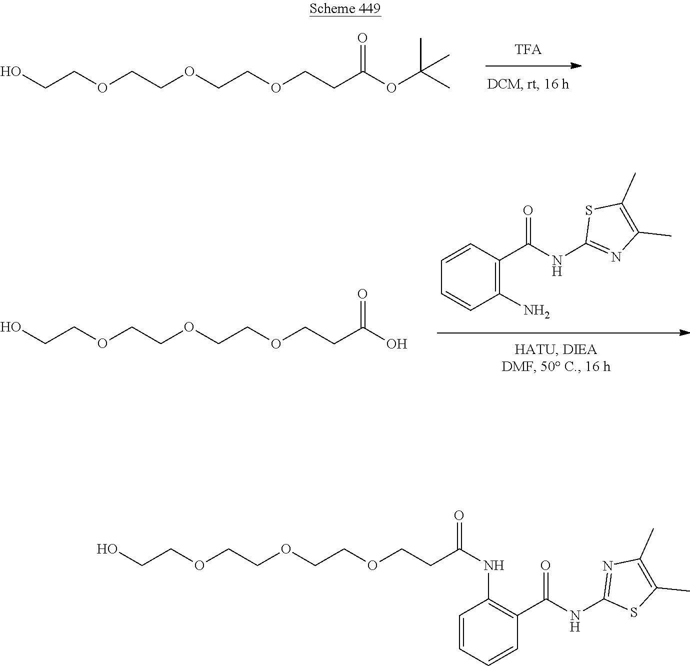 [SMILES: CC(C)(C)OC(=O)CCOCCOCCOCCO ; O=C(O)CCOCCOCCOCCO ; Cc1nc(NC(=O)c2ccccc2N)sc1C ; Cc1nc(NC(=O)c2ccccc2NC(=O)CCOCCOCCOCCO)sc1C]

### Step 1. Synthesis of 3-(2-(2-(2-hydroxyethoxy)ethoxy)ethoxy)propanoic acid

To a solution of tert-butyl 3-(2-(2-(2-hydroxyethoxy)ethoxy)ethoxy)propanoate (800 mg, 2.9 mmol) in DCM (10 mL) was added TFA (3 mL). The mixture was stirred at rt for 6 h. The reaction was monitored by TLC. Upon completion, the mixture was concentrated to provide the title compound (600 mg, 94% yield) as a colorless oil.

### Step 2. Synthesis of N-(4,5-dimethylthiazol-2-yl)-2-(3-(2-(2-(2-hydroxyethoxy)ethoxy)ethoxy)propanamido)benzamide

To a solution of 2-amino-N-(4,5-dimethylthiazol-2-yl)benzamide (400 mg, 1.6 mmol) in DMF (5 mL) were added 3-(2-(2-(2-hydroxyethoxy)ethoxy)ethoxy)propanoic acid (540 mg, 2.4 mmol), HATU (1.23 g, 3.2 mmol) and DIEA (418 mg, 3.2 mmol). The mixture was stirred at 50° C. for 6 h. Upon completion, the mixture was quenched with water and extracted with EtOAc (20 mL×3). The combined organic layers were washed with brine (30 mL), dried over sodium sulfate, filtered and concentrated to dryness. The residue was purified by reverse-phase chromatography (0.1% TFA in H₂O and ACN) to provide the title compound (220 mg, 30% yield) as a white solid. MS (ESI) m/z=451.9 [M+H]⁺.

### Example 450. 2-Acetamido-N-(5-methylpyridin-2-yl)benzamide (B1-31)

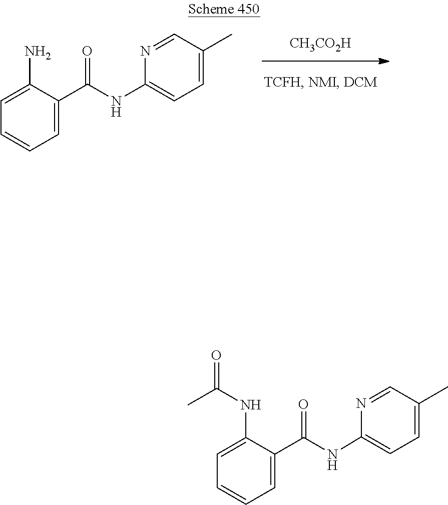

To a mixture of 2-amino-N-(5-methyl-2-pyridyl)benzamide (30 mg, 0.13 mmol) and acetic acid (11.9 mg, 0.19 mmol) in DCM (5 mL) was added NMI (54.1 mg, 0.66 mmol) and TCFH (9.5 mg, 0.26 mmol) at 0° C. After the reaction mixture was warmed to rt and stirred for 2 h, it was concentrated and purified by silica gel flash chromatography and prep-TLC to provide the desired product (9.2 mg, 26% yield) as a white solid. ¹HNMR (400 MHz, DMSO-d₆) δ 10.66 (s, 1H), 10.35 (s, 1H), 8.22 (d, J=0.4 Hz, 1H), 8.04 (d, J=0.8 Hz, 1H), 8.01 (d, J=0.8 Hz, 1H), 7.78 (dd, J=0.4, 0.8 Hz, 1H), 7.67 (dd, J=0.4, 0.8 Hz, 1H), 7.50 (dt, J=0.4, 0.8 Hz, 1H), 7.19 (dt, J=0.4, 0.8 Hz, 1H), 2.29 (s, 3H), 2.05 (s, 3H). MS (ESI) m/z=270.2 [M+H]⁺.

### Example 451. 2-Acetamido-N-(6-methoxypyridazin-3-yl)benzamide (B1-53)

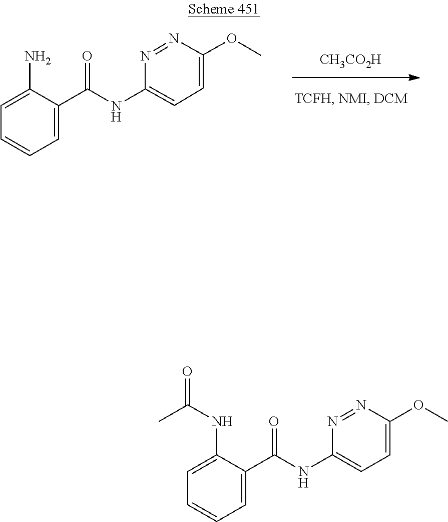

B1-53 was synthesized following the standard procedure for preparing B1-31 (6.5 mg, 28% yield). ¹HNMR (400 MHz, DMSO-d₆) δ 11.22 (s, 1H), 10.28 (s, 1H), 8.20 (d, J=0.8 Hz, 1H), 7.99 (d, J=0.8 Hz, 1H), 7.79 (d, J=0.8 Hz, 1H), 7.52 (t, J=0.8 Hz, 1H), 7.31 (d, J=0.8 Hz, 1H), 7.21 (t, J=0.8 Hz, 1H), 4.02 (s, 3H), 2.04 (s, 3H). MS (ESI) m/z=287.1 [M+H]⁺.

### Example 452. 2-Acetamido-N-(6-(dimethylamino)pyridazin-3-yl)benzamide (B1-71)

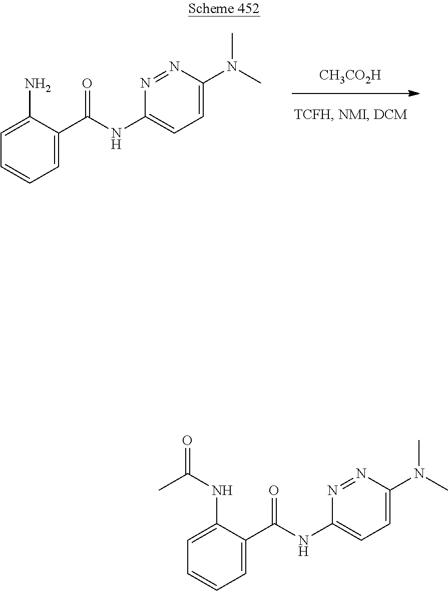 [SMILES: CN(C)c1ccc(NC(=O)c2ccccc2N)nn1 ; "CH3CO2H" ; CC(=O)Nc1ccccc1C(=O)Nc1ccc(N(C)C)nn1]

B1-71 was synthesized following the standard procedure for preparing B1-31 (8.5 mg, 37% yield). ¹HNMR (400 MHz, DMSO-d₆) δ 11.15 (s, 1H), 10.31 (s, 1H), 8.11 (d, J=0.8 Hz, 1H), 7.92 (d, J=0.8 Hz, 1H), 7.75 (d, J=0.8 Hz, 1H), 7.59 (d, J=0.8 Hz, 1H), 7.54 (t, J=0.8 Hz, 1H), 7.23 (t, J=0.8 Hz, 1H), 3.18 (s, 3H), 3.17 (s, 3H), 2.04 (s, 3H). MS (ESI) m/z=300.1 [M+H]⁺.

### Example 453. 2-(5-Aminopentanamido)-N-(5-methylpyridin-2-yl)benzamide (BL1-197)

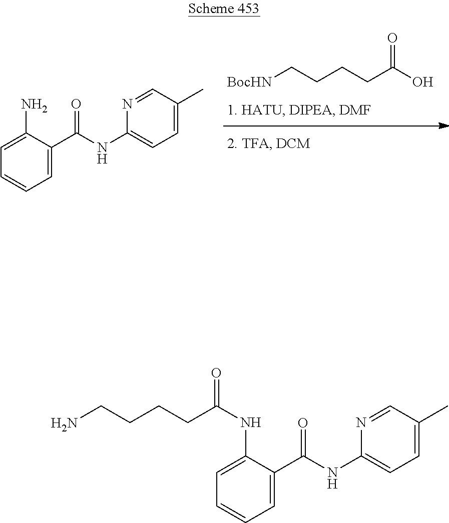 [SMILES: CC(C)(C)OC(=O)NCCCCC(=O)O ; Cc1ccc(NC(=O)c2ccccc2N)nc1 ; Cc1ccc(NC(=O)c2ccccc2NC(=O)CCCCN)nc1]

### Step 1. Synthesis of tert-butyl (5-((2-((5-methylpyridin-2-yl)carbamoyl)phenyl)amino)-5-oxopentyl)carbamate

The title compound was synthesized following the standard procedure for preparing BL1-53 (319 mg, 85% yield). ¹HNMR (400 MHz, DMSO-d₆) δ 10.68 (s, 1H), 10.41 (s, 1H), 8.21 (s, 1H), 8.09 (d, J=8.0 Hz, 1H), 8.00 (d, J=8.4 Hz, 1H), 7.81-7.79 (m, 1H), 7.68-7.65 (m, 1H), 7.52-7.48 (m, 1H), 7.20-7.16 (m, 1H), 6.76-6.74 (m, 1H), 2.92-2.87 (m, 2H), 2.33-2.29 (m, 5H), 1.57-1.50 (m, 2H), 1.43-1.36 (m, 11H). MS (ESI) m/z=427.3 [M+H]⁺.

### Step 2. Synthesis of 2-(5-aminopentanamido)-N-(5-methylpyridin-2-yl)benzamide

The title compound was synthesized following the standard procedure for preparing BL1-53 (11 mg, 71% yield) as TFA salt. MS (ESI) m/z=327.3 [M+H]⁺.

### Example 454. 2-(3-(2-(2-Aminoethoxy)ethoxy)propanamido)-N-(6-cyclopropyl-5-methylpyridin-2-yl)benzamide (BL1-198)

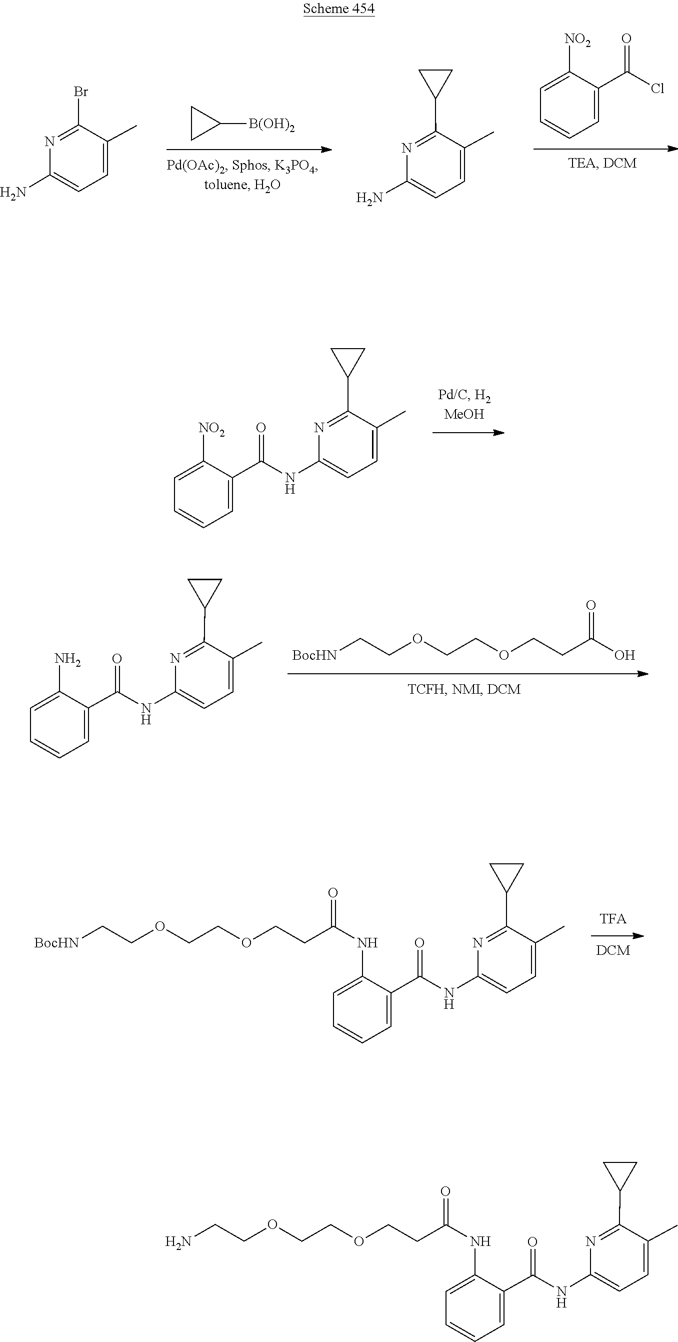

### Step 1. Synthesis of 6-cyclopropyl-5-methylpyridin-2-amine

The title compound was synthesized following the standard procedure for preparing BL1-179 (827 mg, 52% yield) as a yellow solid. MS (ESI) m/z=149.2 [M+H]⁺.

### Step 2. Synthesis of N-(6-cyclopropyl-5-methylpyridin-2-yl)-2-nitrobenzamide

The title compound was synthesized following the standard procedure for preparing BL1-64 (647 mg, 39% yield) as a white solid. MS (ESI) m/z=298.1 [M+H]⁺.

### Step 3. Synthesis of 2-amino-N-(6-cyclopropyl-5-methylpyridin-2-yl)benzamide

The title compound was synthesized following the standard procedure for preparing BL1-55 (201 mg, 34% yield) as a white solid. ¹HNMR (400 MHz, DMSO-d₆) δ 9.86 (brs, 1H), 7.69-7.65 (m, 2H), 7.49 (d, J=8.4 Hz, 1H), 7.22-7.17 (m, 1H), 6.74 (d, J=8.4 Hz, 1H), 6.56 (t, J=8.0 Hz, 1H) 6.35 (brs, 2H), 2.35 (s, 3H), 2.13-2.09 (m, 1H), 1.01-0.97 (m, 2H), 0.91-0.87 (m, 2H). MS (ESI) m/z=268.2 [M+H]⁺.

The remaining steps were performed according to the procedures for preparing BL1-55 to provide the desired product (25 mg, 66% yield over 2 steps) as TFA salt. MS (ESI) m/z=427.3 [M+H]⁺.

### Example 455. N-(4,5-Dimethylthiazol-2-yl)-3-((2-(2-((5-hydroxypentyl)oxy)ethoxy)ethyl)amino)-2-methylbenzamide (BL1-199)

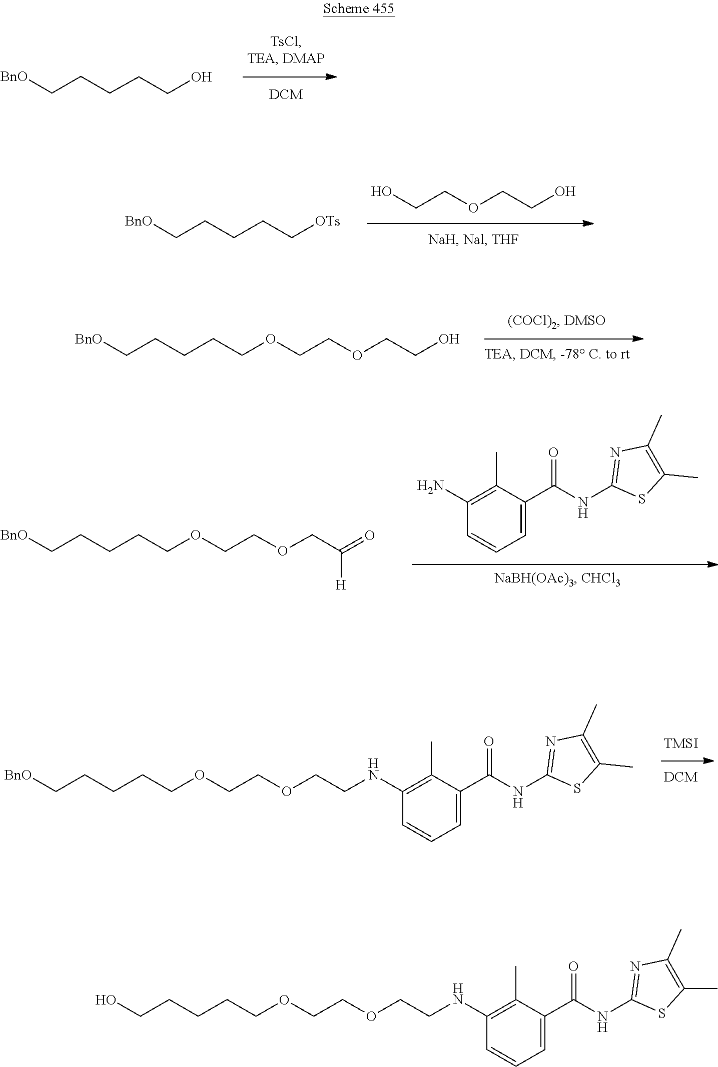

### Step 1. Synthesis of 5-(benzyloxy)pentyl 4-methylbenzenesulfonate

To a solution of 5-(benzyloxy)pentan-1-ol (3.8 g, 19.6 mmol) and Et₃N (3.0 g, 29.4 mmol) in dry DCM (100 mL) were added TsCl (5.6 g, 29.4 mmol) and DMAP (0.2 g, 1.9 mmol) at rt. After stirring at rt overnight, the reaction mixture was diluted with DCM (100 mL), and washed successively with saturated aq. NaHCO₃, water and brine. The organic phase was dried over anhydrous Na₂SO₄, filtered and concentrated under reduced pressure. The residue was purified by silica gel flash chromatography (petroleum ether/ethyl acetate=15:1) to provide the desired product (5.5 g, 81% yield) as a colorless oil.

### Step 2. Synthesis of 2-(2-((5-(benzyloxy)pentyl)oxy)ethoxy)ethan-1-ol

To a stirred solution of 2,2′-oxydiethanol (11.2 g, 105.2 mmol) in THF (300 mL) was added NaH (2.1 g, 52.6 mmol, 60% in mineral oil) portion-wise at 0° C. under nitrogen. The resulting mixture was stirred at 0° C. for 1 h. To the above mixture was added a solution of 5-(benzyloxy)pentyl 4-methylbenzenesulfonate (6.1 g, 17.5 mmol) and NaI (0.3 g, 1.7 mmol) in THF (10 mL) at rt. After stirring at rt overnight, the reaction was quenched with water (50 mL) slowly, then diluted with EtOAc (125 mL) and saturated brine (50 mL). The aqueous layer was separated and further extracted with EtOAc (75 mL). The combined organic layers were dried over anhydrous Na₂SO₄, filtered and concentrated under reduced pressure. The residue was purified by silica gel flash chromatography (petroleum ether/ethyl acetate=3:1) to provide the desired product (3.2 g, 65% yield) as a brown oil.

### Step 3. Synthesis of 2-(2-((5-(benzyloxy)pentyl)oxy)ethoxy)acetaldehyde

To a solution of (COCl)₂ (900 mg, 7.08 mmol) in anhydrous CH₂Cl₂ (20 mL) was added DMSO (830 mg, 10.62 mmol) at −78° C. under nitrogen. After stirring at −78° C. for 50 min, a solution of 2-(2-((5-(benzyloxy)pentyl)oxy)ethoxy)ethanol (1.0 g, 3.54 mmol) in anhydrous CH₂Cl₂ (10 mL) was added dropwise. The mixture was stirred at the same temperature for 50 min, then Et₃N (1.8 g, 17.7 mmol) was added dropwise. After stirring at −78° C. for another 30 min, the reaction mixture was warmed to rt and stirred for 2 h. The reaction mixture was acidified with 1 N HCl solution, then extracted with CH₂Cl₂ (3×400 mL). The combined organic layers were dried over anhydrous Na₂SO₄, filtered and concentrated under reduced pressure to provide the desired product (900 mg, crude) as a colorless oil which was used in the next step directly without further purification.

### Step 4. Synthesis of 3-((2-(2-((5-(benzyloxy)pentyl)oxy)ethoxy)ethyl)amino)-N-(4,5-dimethylthiazol-2-yl)-2-methylbenzamide

To a solution of 3-amino-N-(4,5-dimethylthiazol-2-yl)-2-methylbenzamide (550 mg, 2.1 mmol) and 2-(2-((5-(benzyloxy)pentyl)oxy)ethoxy)acetaldehyde (900 mg, crude) in CHCl₃ (20 mL) was added NaBH(OAc)₃ (900 mg, 4.2 mmol) at rt. After the reaction mixture was stirred at rt overnight, it was quenched with aq. NaHCO₃ (10 mL) and extracted with DCM (10 mL×3). The combined organic layers were washed with brine, dried over Na₂SO₄, filtered and concentrated under reduced pressure. The residue was purified by silica gel flash chromatography (petroleum ether/ethyl acetate=2:1) to provide the desired product (400 mg, 36% yield) as a yellow oil. MS (ESI) m/z=526.1 [M+H]⁺.

### Step 5. Synthesis of N-(4,5-dimethylthiazol-2-yl)-3-((2-(2-((5-hydroxypentyl)oxy)ethoxy)ethyl)amino)-2-methylbenzamide

To a solution of 3-((2-(2-((5-(benzyloxy)pentyl)oxy)ethoxy)ethyl)amino)-N-(4,5-dimethylthiazol-2-yl)-2-methylbenzamide (390 mg, 0.74 mmol) in DCM (20 mL) was added a solution of TMSI (380 mg, 1.85 mmol) in DCM (2 mL) at rt. After the reaction mixture was stirred at rt for 2 h, it was quenched with H₂O (10 mL) and extracted with DCM (10 mL×3). The combined organic layers were washed with Na₂S₂O₃ solution, dried over Na₂SO₄, filtered and concentrated under reduced pressure. The residue was purified by silica gel flash chromatography (ethyl acetate) to provide the desired product (170 mg, 52% yield) as a pale-yellow solid. ¹HNMR (400 MHz, DMSO-d₆) δ 12.05 (s, 1H), 7.10 (t, J=8.0 Hz, 1H), 6.72-6.59 (m, 2H), 4.94 (t, J=5.6 Hz, 1H), 4.32 (t, J=5.2 Hz, 1H), 3.61 (t, J=6.0 MHz, 1H), 3.57-3.54 (m, 3H), 3.50-3.47 (m, 2H), 3.39-3.34 (m, 4H), 3.29-3.26 (m, 2H), 2.26 (s, 3H), 2.17 (s, 3H), 2.05 (s, 3H), 1.54-1.36 (m, 4H), 1.31-1.23 (m, 2H). MS (ESI) m/z=436.3 [M+H]⁺.

### Example 456. 2-(5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentanamido)-N-(5-methylpyridin-2-yl)benzamide (CPD-269)

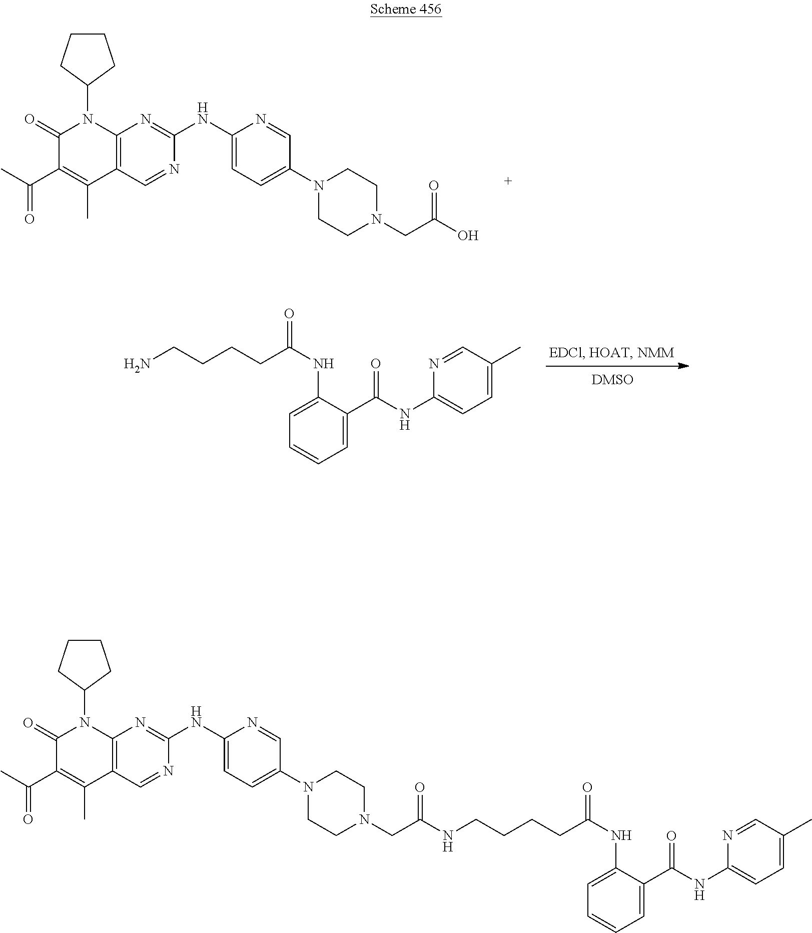 [SMILES: CC(=O)c1c(C)c2cnc(Nc3ccc(N4CCN(CC(=O)O)CC4)cn3)nc2n(C2CCCC2)c1=O ; "+" ; Cc1ccc(NC(=O)c2ccccc2NC(=O)CCCCN)nc1 ; CC(=O)c1c(C)c2cnc(Nc3ccc(N4CCN(CC(=O)NCCCCC(=O)Nc5ccccc5C(=O)Nc5ccc(C)cn5)CC4)cn3)nc2n(C2CCCC2)c1=O]

CPD-269 was synthesized following the standard procedure for preparing CPD-008 (3.4 mg, 15% yield) as a yellow solid. MS (ESI) m/z 814.6 [M+H]⁺.

### Example 457. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(6-cyclopropyl-5-methylpyridin-2-yl)benzamide (CPD-270)

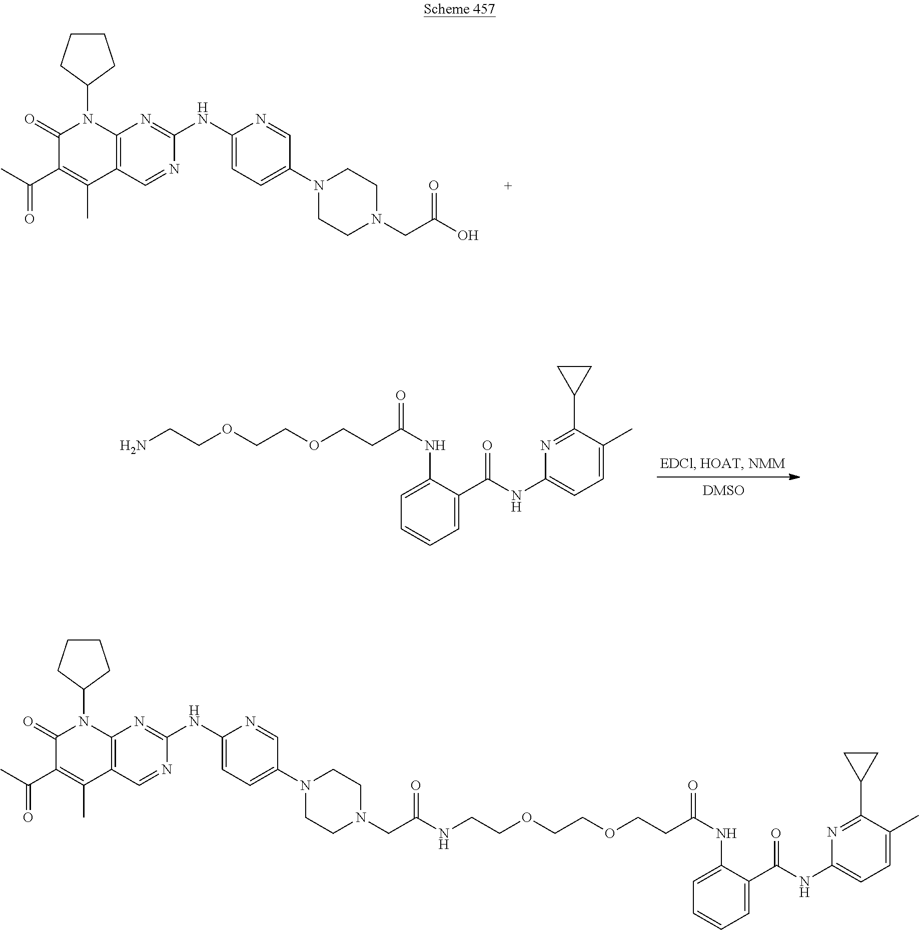 [SMILES: CC(=O)c1c(C)c2cnc(Nc3ccc(N4CCN(CC(=O)O)CC4)cn3)nc2n(C2CCCC2)c1=O ; "+" ; Cc1ccc(NC(=O)c2ccccc2NC(=O)CCOCCOCCN)nc1C1CC1 ; CC(=O)c1c(C)c2cnc(Nc3ccc(N4CCN(CC(=O)NCCOCCOCCC(=O)Nc5ccccc5C(=O)Nc5ccc(C)c(C6CC6)n5)CC4)cn3)nc2n(C2CCCC2)c1=O]

CPD-270 was synthesized following the standard procedure for preparing CPD-008 (11.6 mg, 21% yield) as a yellow solid. MS (ESI) m/z 914.5 [M+H]⁺.

### Example 458. 3-((2-(2-((5-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)pentyl)oxy)ethoxy)ethyl)amino)-N-(4,5-dimethylthiazol-2-yl)-2-methylbenzamide (CPD-271)

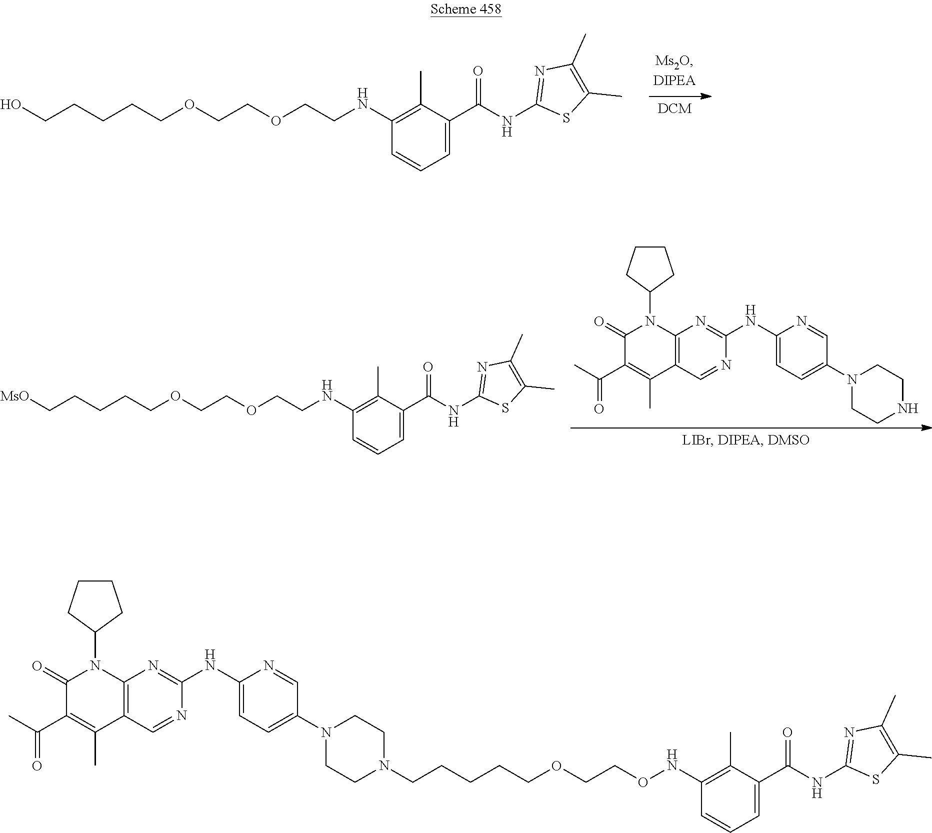

CPD-271 was synthesized following the standard procedure for preparing CPD-078 (9.3 mg, 23% yield over 2 steps) as a yellow solid. MS (ESI) m/z 865.5 [M+H]⁺.

### Example 459. N-(4,5-Dimethylthiazol-2-yl)-2-(2-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)acetamido)benzamide (CPD-272)

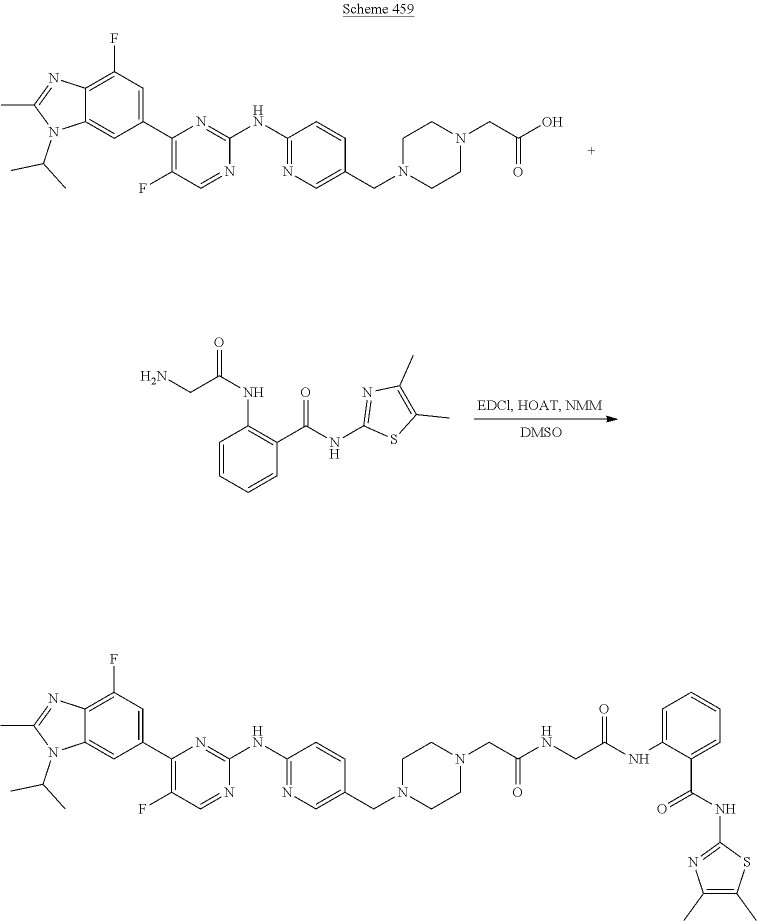

CPD-272 was synthesized following the standard procedure for preparing CPD-008 (3.1 mg, 25% yield) as a yellow solid. MS (ESI) m/z 823.4 [M+H]⁺.

### Example 460. N-(4,5-Dimethylthiazol-2-yl)-2-(3-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)propanamido)benzamide (CPD-273)

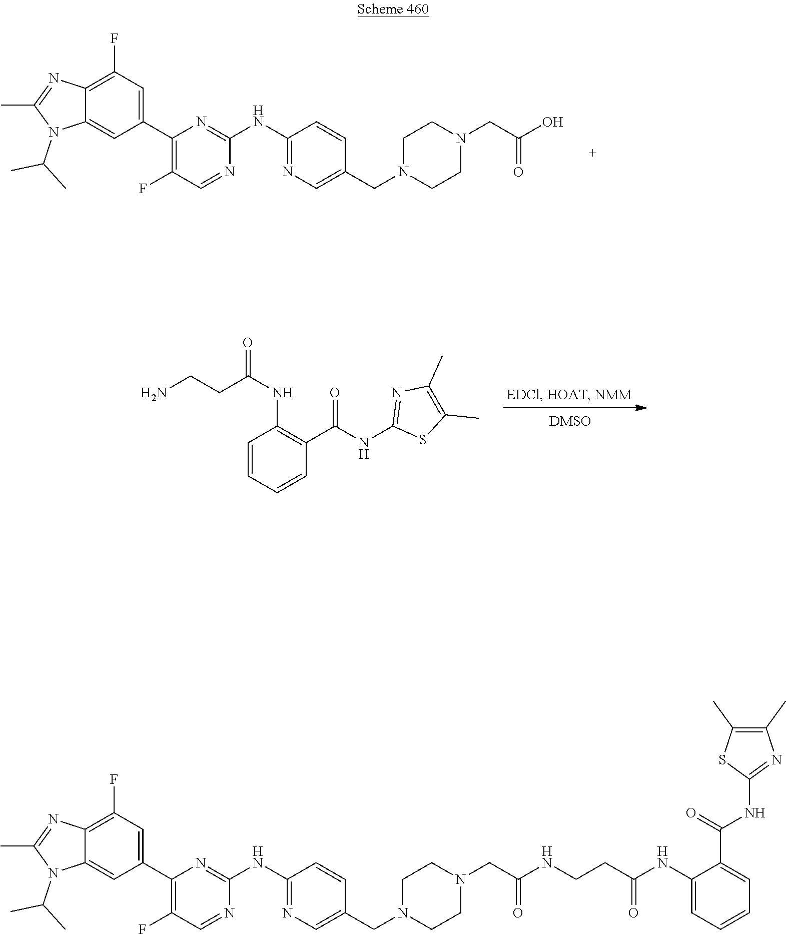

CPD-273 was synthesized following the standard procedure for preparing CPD-008 (3.2 mg, 24% yield) as a yellow solid. MS (ESI) m/z 837.5 [M+H]⁺.

### Example 461. N-(4,5-Dimethylthiazol-2-yl)-2-(4-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)butanamido)benzamide (CPD-274)

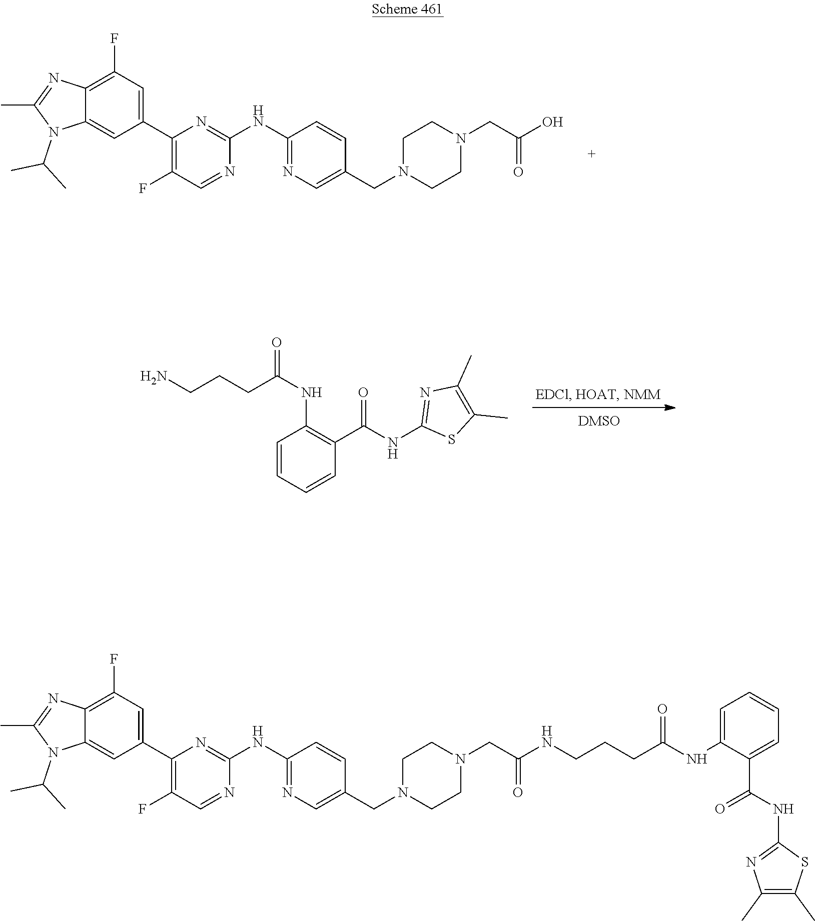

CPD-274 was synthesized following the standard procedure for preparing CPD-008 (4.1 mg, 31% yield) as a yellow solid. MS (ESI) m/z 851.5 [M+H]⁺.

### Example 462. N-(4,5-Dimethylthiazol-2-yl)-2-(5-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)pentanamido)benzamide (CPD-275)

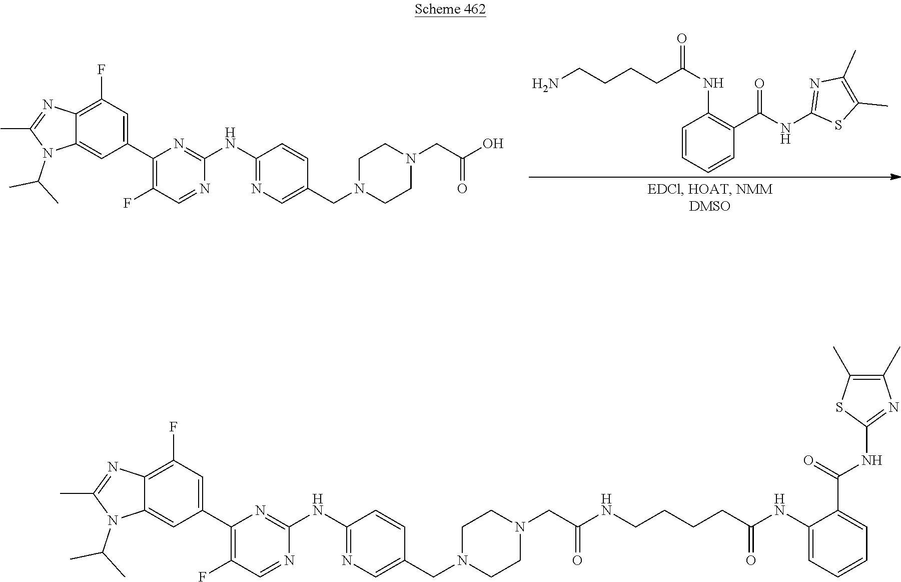

CPD-275 was synthesized following the standard procedure for preparing CPD-008 (2.8 mg, 21% yield) as a yellow solid. MS (ESI) m/z 865.5 [M+H]⁺.

### Example 463. N-(4,5-Dimethylthiazol-2-yl)-2-(6-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)hexanamido)benzamide (CPD-276)

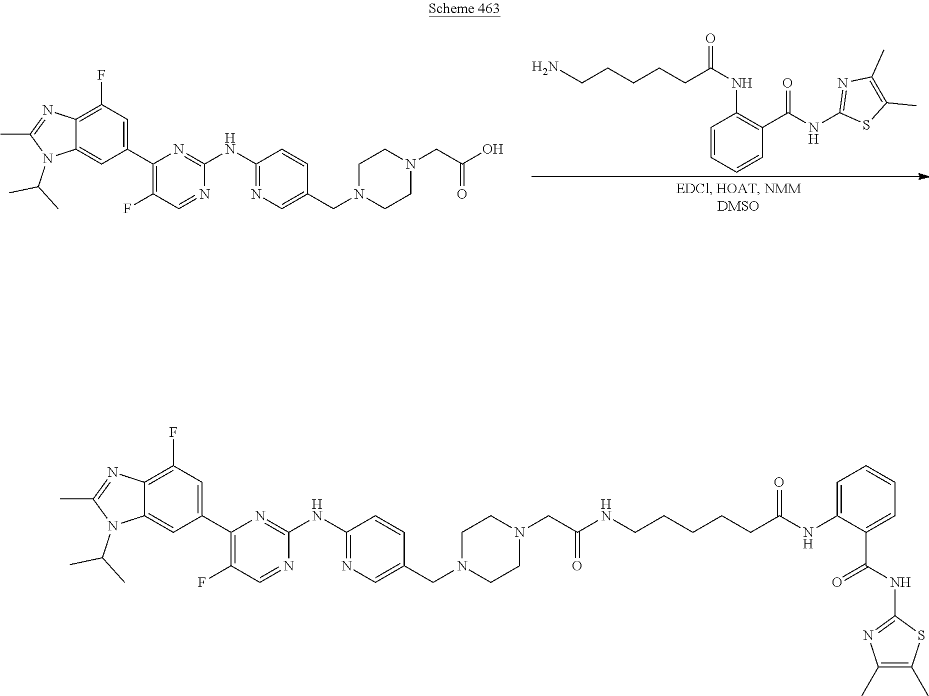

CPD-276 was synthesized following the standard procedure for preparing CPD-008 (2.3 mg, 17% yield) as a yellow solid. MS (ESI) m/z 879.5 [M+H]⁺.

### Example 464. N-(4,5-Dimethylthiazol-2-yl)-2-(7-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)heptanamido)benzamide (CPD-277)

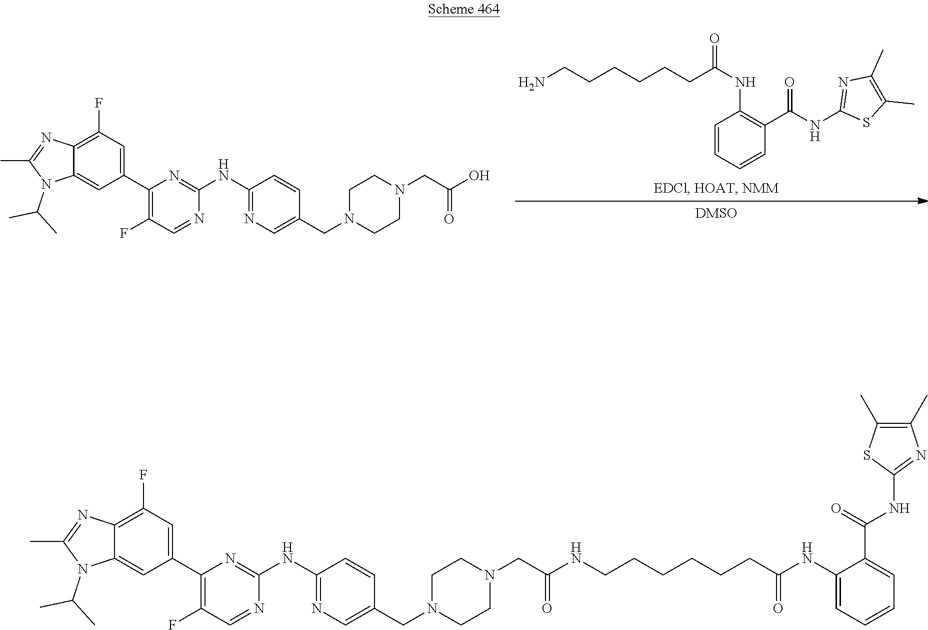

CPD-277 was synthesized following the standard procedure for preparing CPD-008 (3.1 mg, 22% yield) as a yellow solid. MS (ESI) m/z 893.5 [M+H]⁺.

### Example 465. N-(4,5-Dimethylthiazol-2-yl)-2-(8-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)octanamido)benzamide (CPD-278)

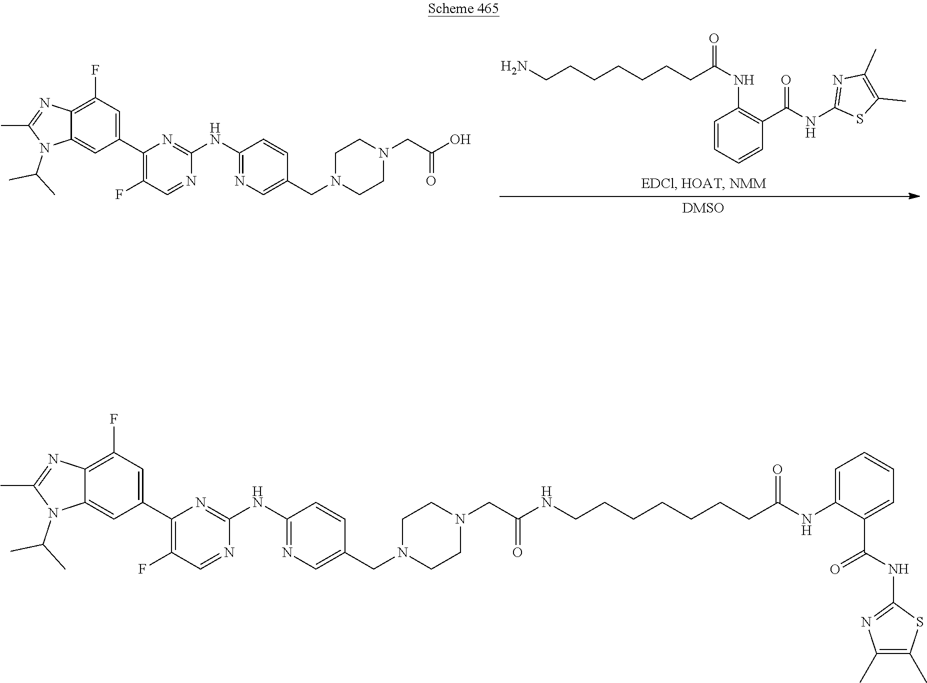

CPD-278 was synthesized following the standard procedure for preparing CPD-008 (3.4 mg, 24% yield) as a yellow solid. MS (ESI) m/z 907.5 [M+H]⁺.

### Example 466. N-(4,5-Dimethylthiazol-2-yl)-2-(9-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)nonanamido)benzamide (CPD-279)

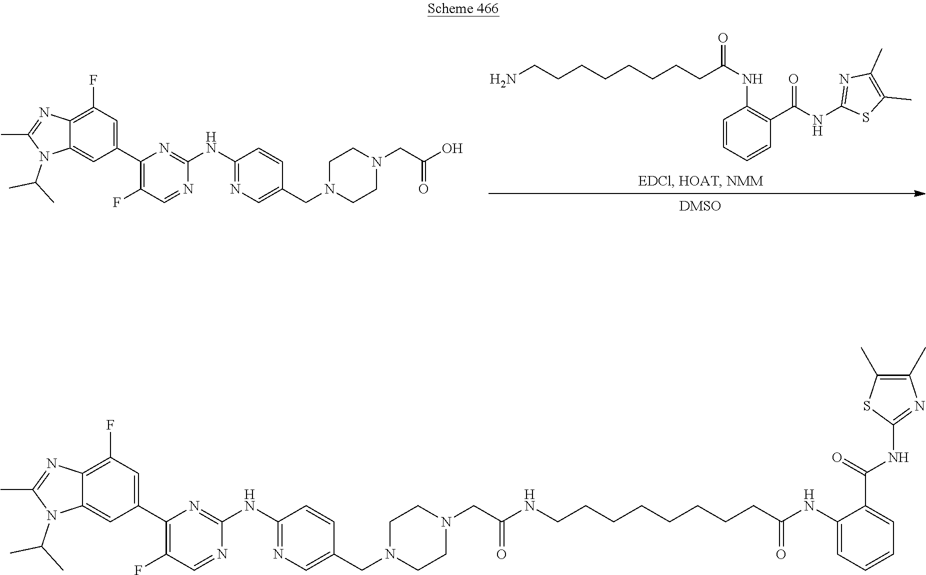

CPD-279 was synthesized following the standard procedure for preparing CPD-008 (2.7 mg, 19% yield) as a yellow solid. MS (ESI) m/z 921.5 [M+H]⁺.

### Example 467. N-(4,5-Dimethylthiazol-2-yl)-2-(3-(2-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)ethoxy)propanamido)benzamide (CPD-280)

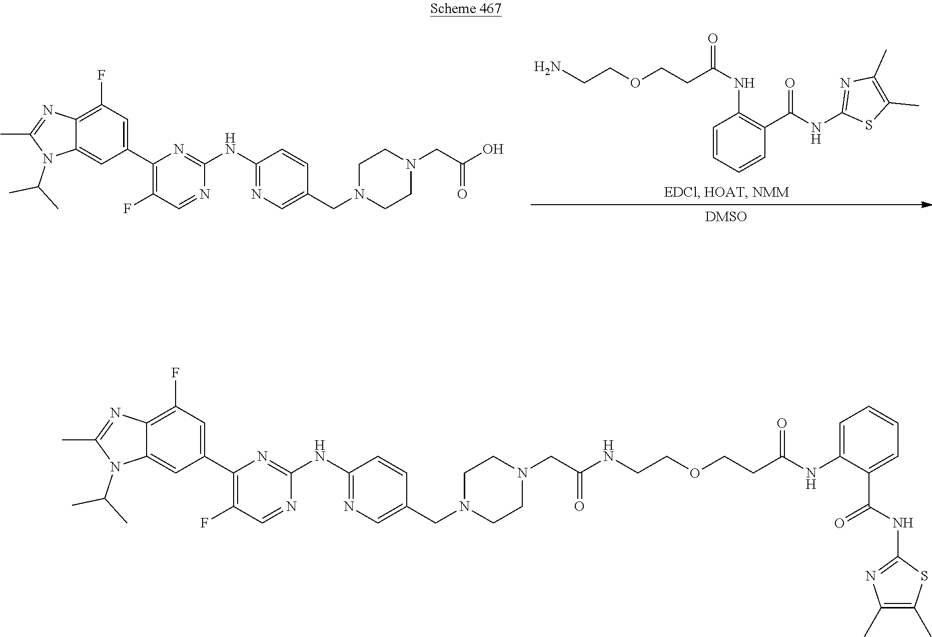

CPD-280 was synthesized following the standard procedure for preparing CPD-008 (3.6 mg, 26% yield) as a yellow solid. MS (ESI) m/z 881.5 [M+H]⁺.

### Example 468. N-(4,5-Dimethylthiazol-2-yl)-2-(3-(2-(2-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)ethoxy)ethoxy)propanamido)benzamide (CPD-281)

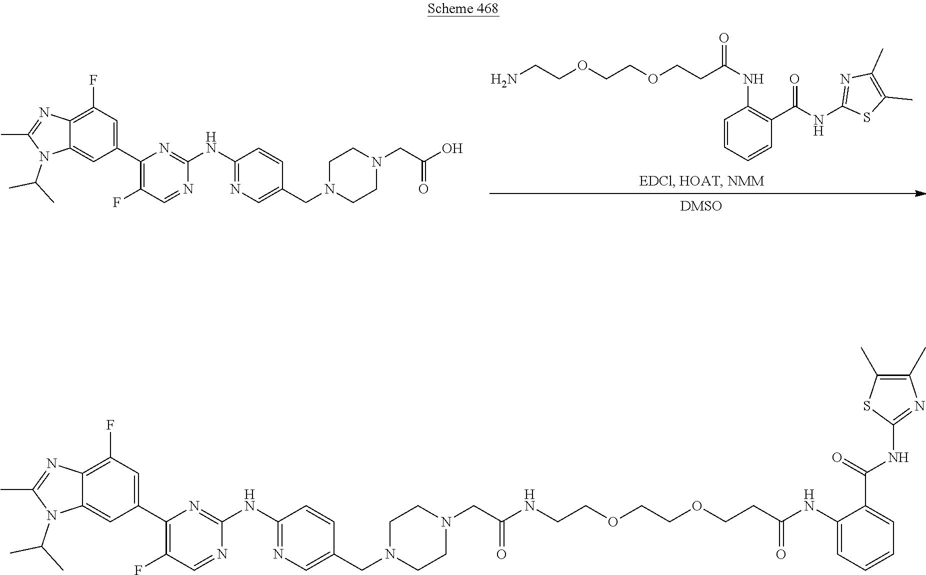

CPD-281 was synthesized following the standard procedure for preparing CPD-008 (4.2 mg, 29% yield) as a yellow solid. MS (ESI) m/z 925.6 [M+H]⁺.

### Example 469. N-(4,5-Dimethylthiazol-2-yl)-2-(1-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxo-6,9,12-trioxa-3-azapentadecan-15-amido)benzamide (CPD-282)

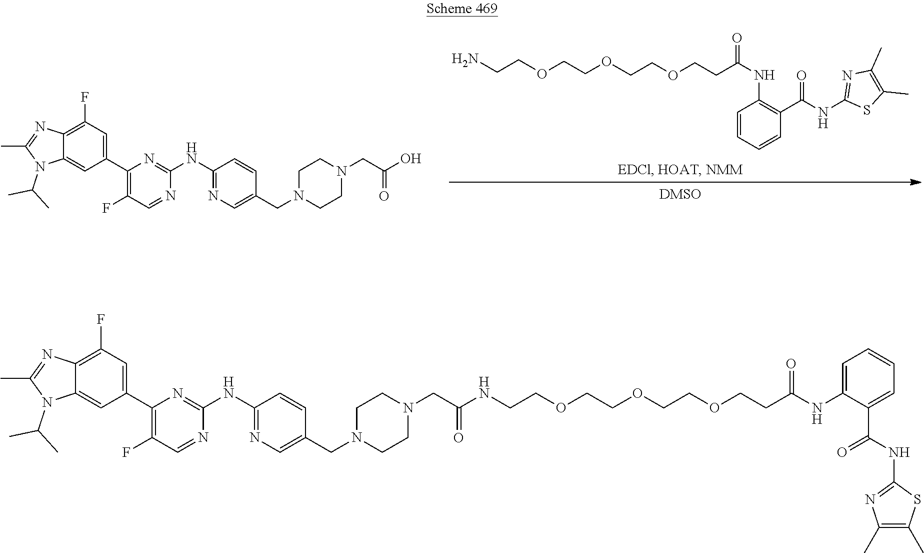

CPD-282 was synthesized following the standard procedure for preparing CPD-008 (5.2 mg, 34% yield) as a yellow solid. MS (ESI) m/z 969.5 [M+H]⁺.

### Example 470. N-(4,5-Dimethylthiazol-2-yl)-2-(1-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxo-6,9,12,15-tetraoxa-3-azaoctadecan-18-amido)benzamide (CPD-283)

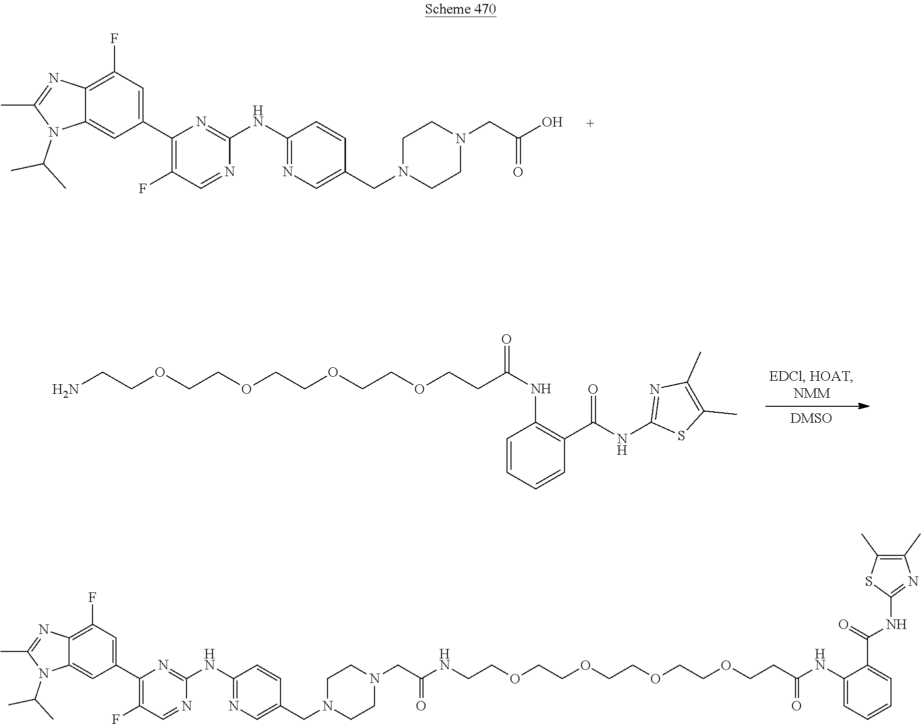

CPD-283 was synthesized following the standard procedure for preparing CPD-008 (3.1 mg, 20% yield) as a yellow solid. MS (ESI) m/z 1013.5 [M+H]⁺.

### Example 471. N-(4,5-Dimethylthiazol-2-yl)-2-(1-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxo-6,9,12,15,18-pentaoxa-3-azahenicosan-21-amido)benzamide (CPD-284)

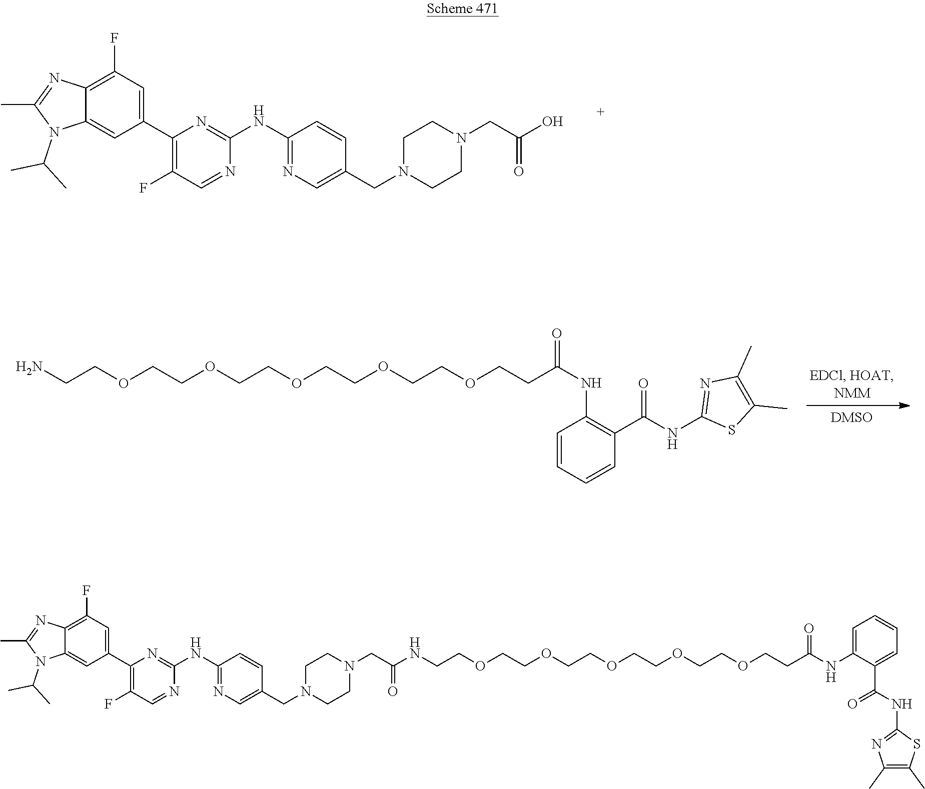

CPD-284 was synthesized following the standard procedure for preparing CPD-008 (4.0 mg, 24% yield) as a yellow solid. MS (ESI) m/z 1058.0 [M+H]⁺.

### Example 472. N-(4,5-Dimethylthiazol-2-yl)-2-(1-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxo-6,9,12,15,18,21-hexaoxa-3-azatetracosan-24-amido)benzamide (CPD-285)

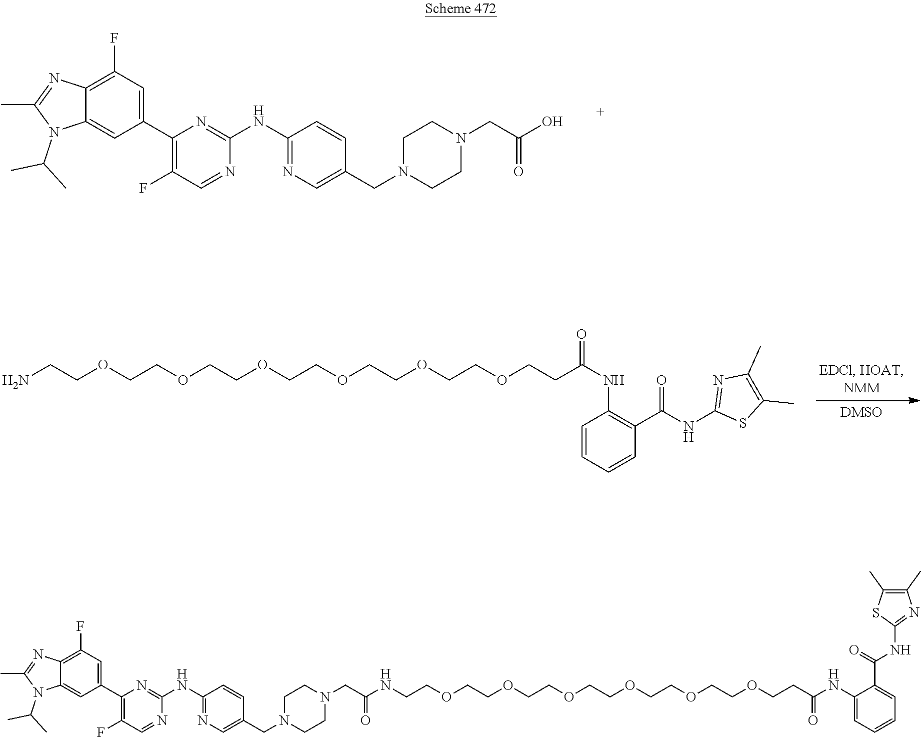

CPD-285 was synthesized following the standard procedure for preparing CPD-008 (3.5 mg, 20% yield) as a yellow solid. MS (ESI) m/z 1102.1 [M+H]⁺.

### Example 473. 7-Cyclopentyl-2-((5-(4-(2-((2-((2-((4,5-dimethylthiazol-2-yl)carbamoyl)phenyl)amino)-2-oxoethyl)amino)-2-oxoethyl)piperazin-1-yl)pyridin-2-yl)amino)-N,N-dimethyl-7H-pyrrolo[2,3-d]pyrimidine-6-carboxamide (CPD-286)

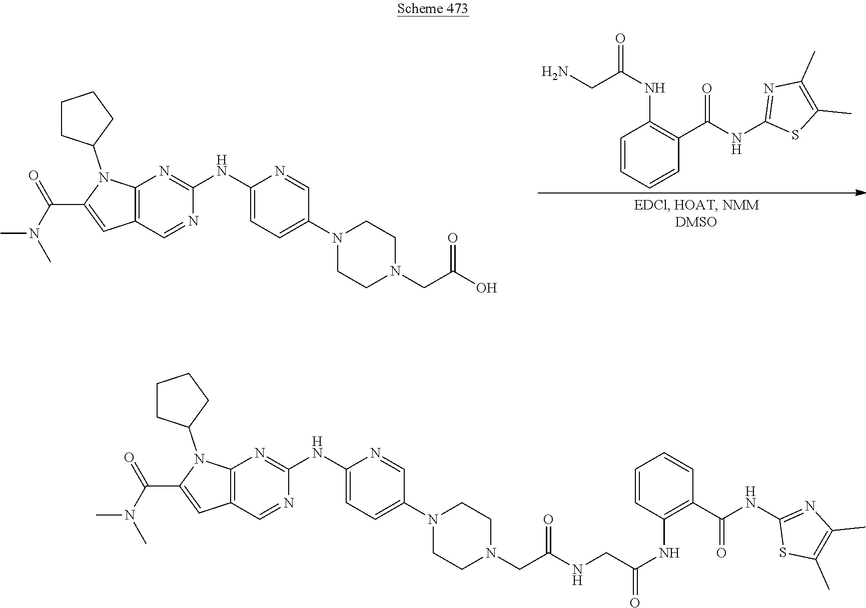

CPD-286 was synthesized following the standard procedure for preparing CPD-008 (3.6 mg, 47% yield) as a yellow solid. MS (ESI) m/z 779.4 [M+H]⁺.

### Example 474. 7-Cyclopentyl-2-((5-(4-(2-((3-((2-((4,5-dimethylthiazol-2-yl)carbamoyl)phenyl)amino)-3-oxopropyl)amino)-2-oxoethyl)piperazin-1-yl)pyridin-2-yl)amino)-N,N-dimethyl-7H-pyrrolo[2,3-d]pyrimidine-6-carboxamide (CPD-287)

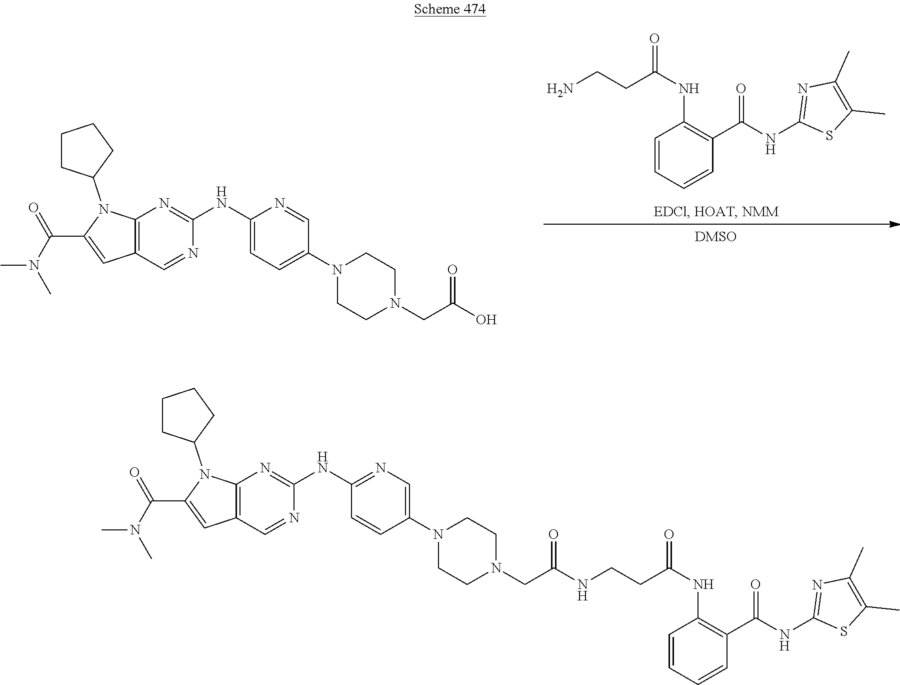

CPD-287 was synthesized following the standard procedure for preparing CPD-008 (4.5 mg, 37% yield) as a yellow solid. MS (ESI) m/z 793.5 [M+H]⁺.

### Example 475. 7-Cyclopentyl-2-((5-(4-(2-((4-((2-((4,5-dimethylthiazol-2-yl)carbamoyl)phenyl)amino)-4-oxobutyl)amino)-2-oxoethyl)piperazin-1-yl)pyridin-2-yl)amino)-N,N-dimethyl-7H-pyrrolo[2,3-d]pyrimidine-6-carboxamide (CPD-288)

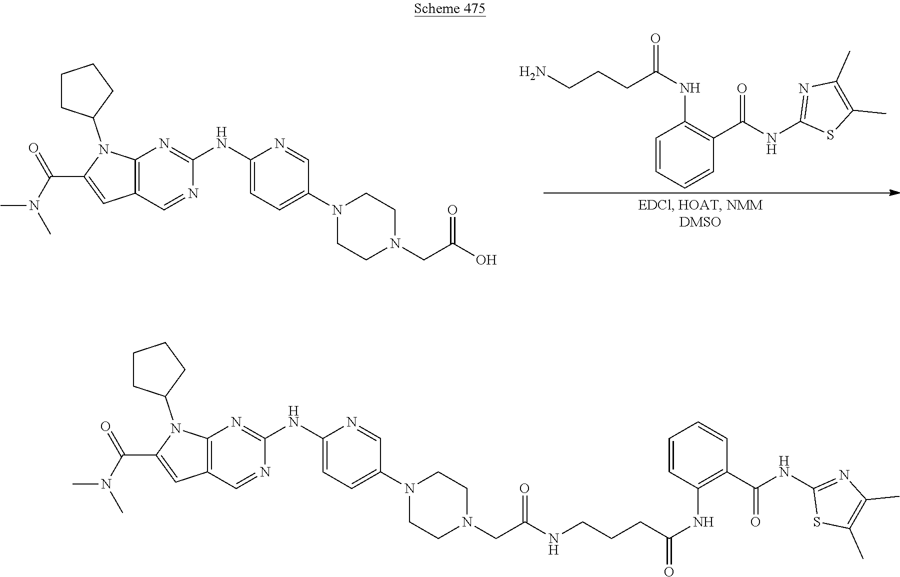

CPD-288 was synthesized following the standard procedure for preparing CPD-008 (4.5 mg, 37% yield) as a yellow solid. MS (ESI) m/z 807.4 [M+H]⁺.

### Example 476. 7-Cyclopentyl-2-((5-(4-(2-((5-((2-((4,5-dimethylthiazol-2-yl)carbamoyl)phenyl)amino)-5-oxopentyl)amino)-2-oxoethyl)piperazin-1-yl)pyridin-2-yl)amino)-N,N-dimethyl-7H-pyrrolo[2,3-d]pyrimidine-6-carboxamide (CPD-289)

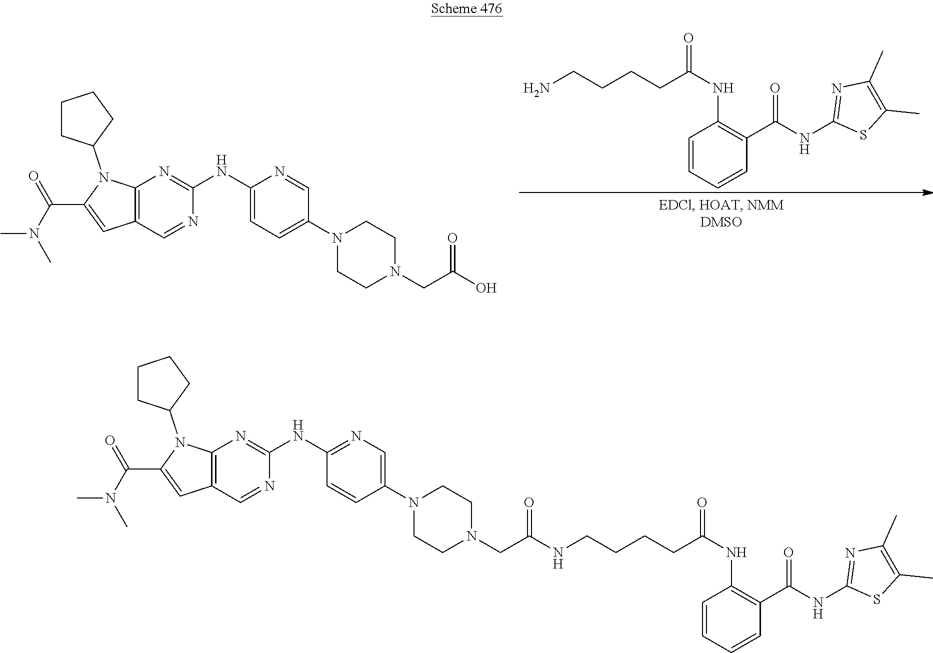

CPD-289 was synthesized following the standard procedure for preparing CPD-008 (5.1 mg, 41% yield) as a yellow solid. MS (ESI) m/z 821.5 [M+H]⁺.

### Example 477. 7-Cyclopentyl-2-((5-(4-(2-((6-((2-((4,5-dimethylthiazol-2-yl)carbamoyl)phenyl)amino)-6-oxohexyl)amino)-2-oxoethyl)piperazin-1-yl)pyridin-2-yl)amino)-N,N-dimethyl-7H-pyrrolo[2,3-d]pyrimidine-6-carboxamide (CPD-290)

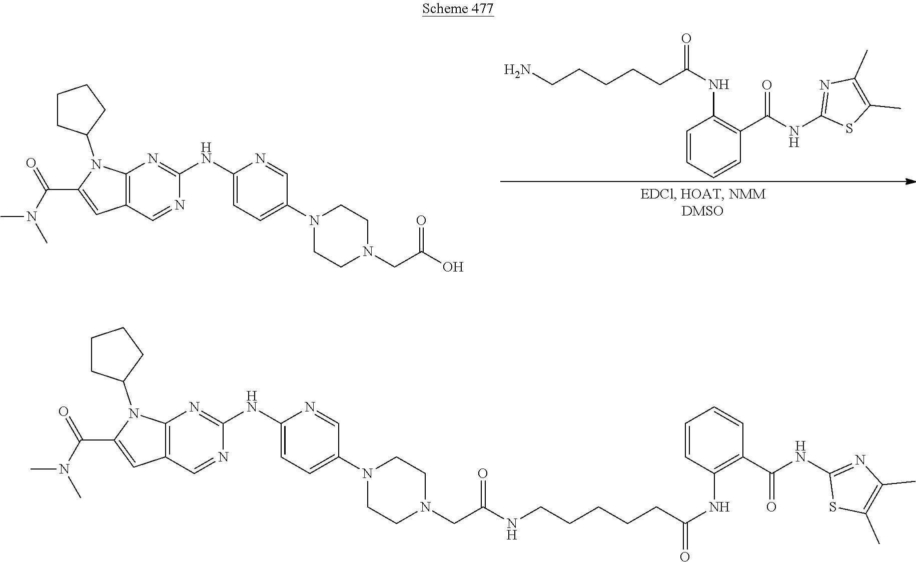

CPD-290 was synthesized following the standard procedure for preparing CPD-008 (5.1 mg, 40% yield) as a yellow solid. MS (ESI) m/z 835.5 [M+H]⁺.

### Example 478. 7-Cyclopentyl-2-((5-(4-(2-((7-((2-((4,5-dimethylthiazol-2-yl)carbamoyl)phenyl)amino)-7-oxoheptyl)amino)-2-oxoethyl)piperazin-1-yl)pyridin-2-yl)amino)-N,N-dimethyl-7H-pyrrolo[2,3-d]pyrimidine-6-carboxamide (CPD-291)

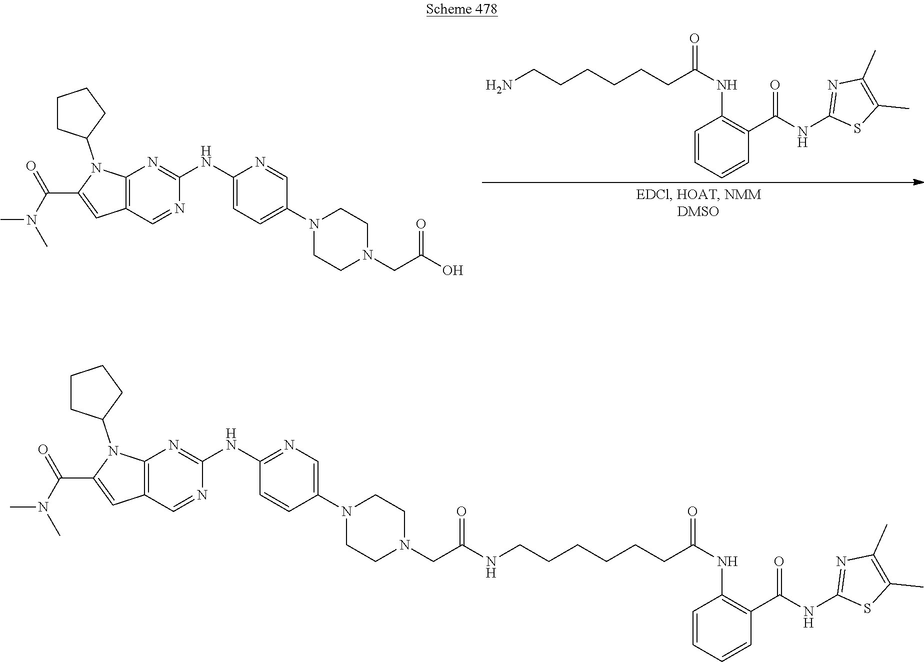

CPD-291 was synthesized following the standard procedure for preparing CPD-008 (5.1 mg, 39% yield) as a yellow solid. MS (ESI) m/z 849.5 [M+H]⁺.

### Example 479. 7-Cyclopentyl-2-((5-(4-(2-((8-((2-((4,5-dimethylthiazol-2-yl)carbamoyl)phenyl)amino)-8-oxooctyl)amino)-2-oxoethyl)piperazin-1-yl)pyridin-2-yl)amino)-N,N-dimethyl-7H-pyrrolo[2,3-d]pyrimidine-6-carboxamide (CPD-292)

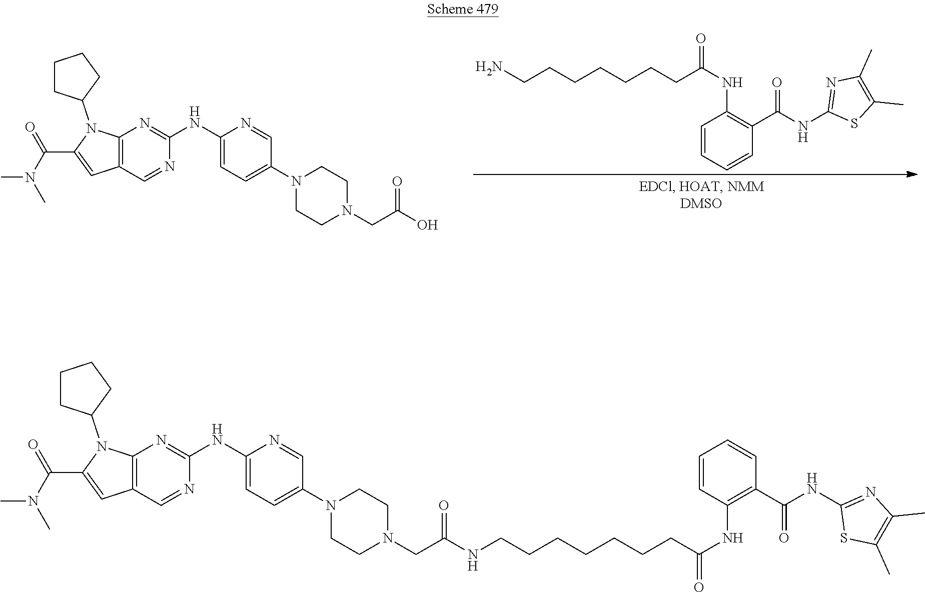

CPD-292 was synthesized following the standard procedure for preparing CPD-008 (4.5 mg, 34% yield) as a yellow solid. MS (ESI) m/z 863.5 [M+H]⁺.

### Example 480. 7-Cyclopentyl-2-((5-(4-(2-((9-((2-((4,5-dimethylthiazol-2-yl)carbamoyl)phenyl)amino)-9-oxononyl)amino)-2-oxoethyl)piperazin-1-yl)pyridin-2-yl)amino)-N,N-dimethyl-7H-pyrrolo[2,3-d]pyrimidine-6-carboxamide (CPD-293)

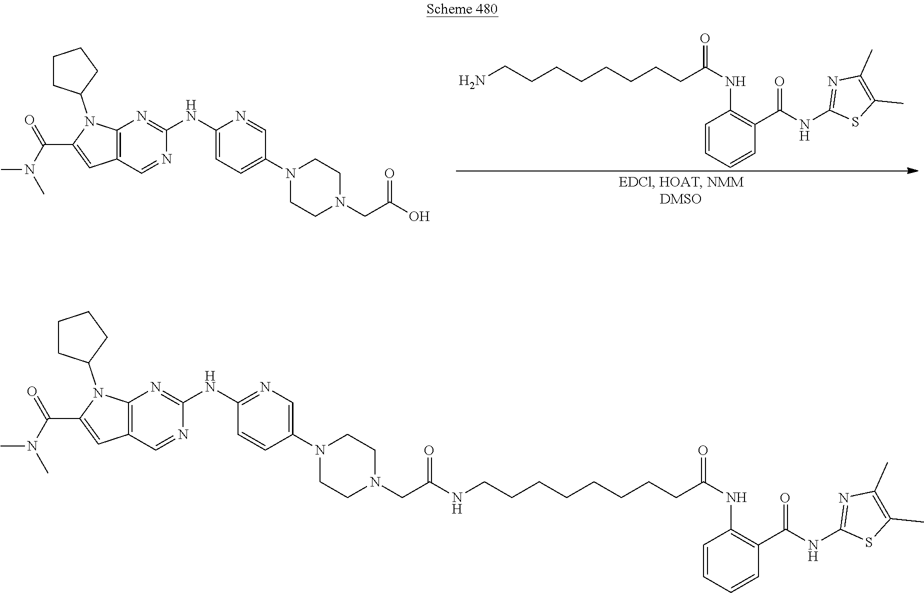

CPD-293 was synthesized following the standard procedure for preparing CPD-008 (4.2 mg, 31% yield) as a yellow solid. MS (ESI) m/z 877.5 [M+H]⁺.

### Example 481. 7-Cyclopentyl-2-((5-(4-(2-((2-(3-((2-((4,5-dimethylthiazol-2-yl)carbamoyl)phenyl)amino)-3-oxopropoxy)ethyl)amino)-2-oxoethyl)piperazin-1-yl)pyridin-2-yl)amino)-N,N-dimethyl-7H-pyrrolo[2,3-d]pyrimidine-6-carboxamide (CPD-294)

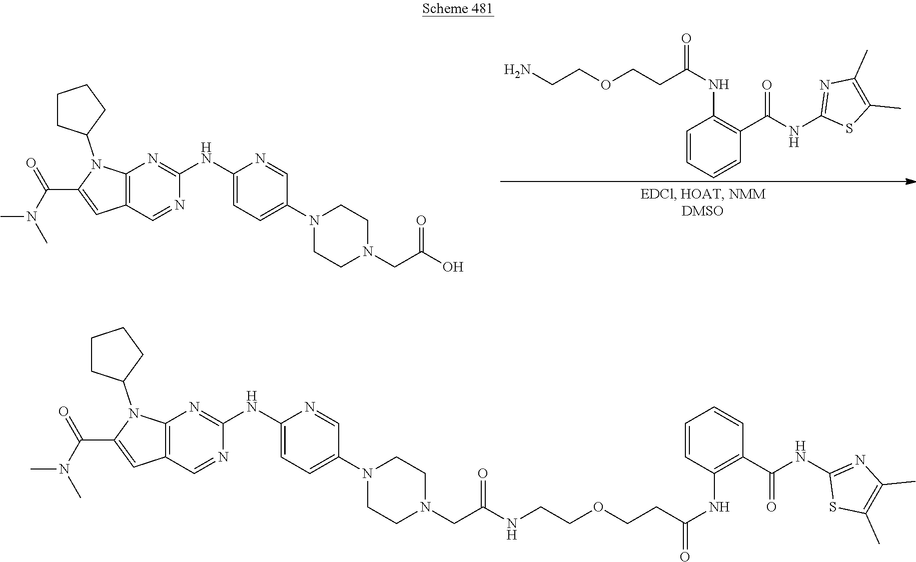

CPD-294 was synthesized following the standard procedure for preparing CPD-008 (3.1 mg, 26% yield) as a yellow solid. MS (ESI) m/z 837.5 [M+H]⁺.

### Example 482. 7-Cyclopentyl-2-((5-(4-(2-((2-(2-(3-((2-((4,5-dimethylthiazol-2-yl)carbamoyl)phenyl)amino)-3-oxopropoxy)ethoxy)ethyl)amino)-2-oxoethyl)piperazin-1-yl)pyridin-2-yl)amino)-N,N-dimethyl-7H-pyrrolo[2,3-d]pyrimidine-6-carboxamide (CPD-295)

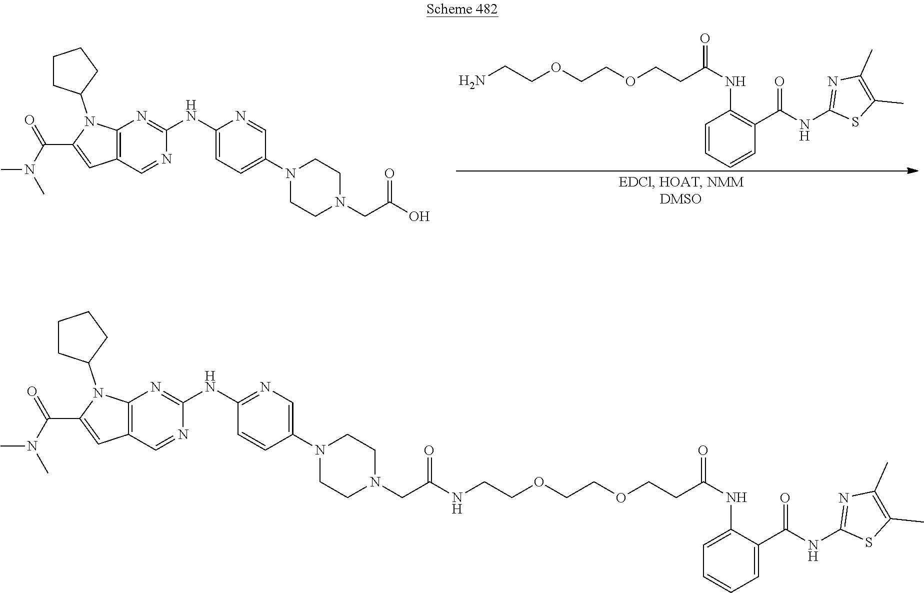

CPD-295 was synthesized following the standard procedure for preparing CPD-008 (3.5 mg, 28% yield) as a yellow solid. MS (ESI) m/z 881.5 [M+H]⁺.

### Example 483. 7-Cyclopentyl-2-((5-(4-(15-((2-((4,5-dimethylthiazol-2-yl)carbamoyl)phenyl)amino)-2,15-dioxo-6,9,12-trioxa-3-azapentadecyl)piperazin-1-yl)pyridin-2-yl)amino)-N,N-dimethyl-7H-pyrrolo[2,3-d]pyrimidine-6-carboxamide (CPD-296)

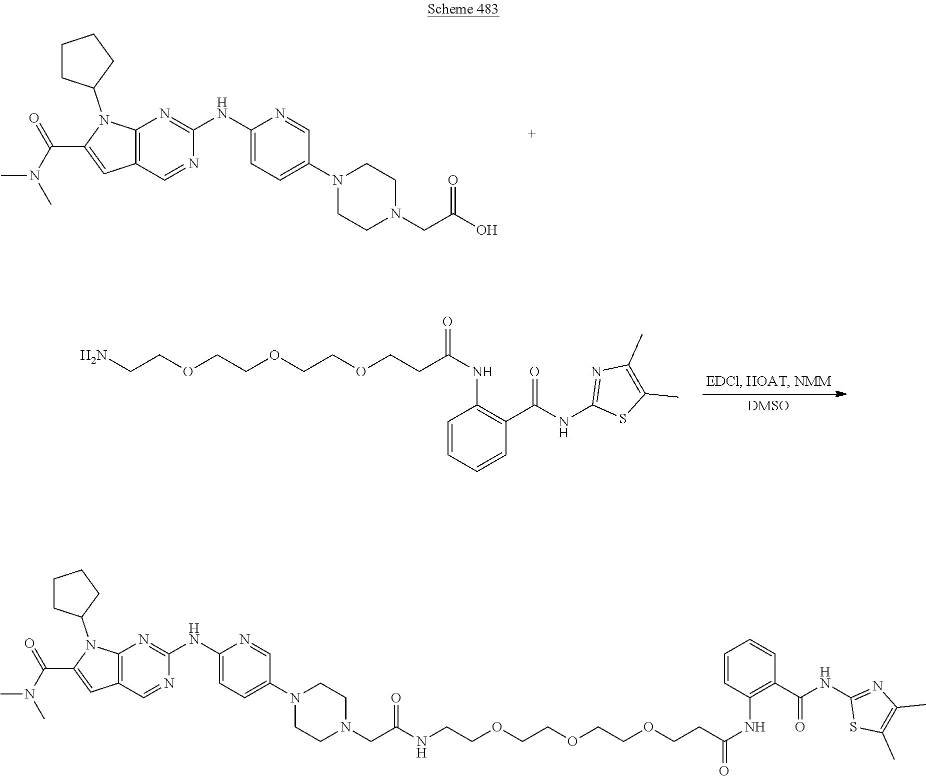

CPD-296 was synthesized following the standard procedure for preparing CPD-008 (3.5 mg, 27% yield) as a yellow solid. MS (ESI) m/z 925.5 [M+H]⁺.

### Example 484. 7-Cyclopentyl-2-((5-(4-(18-((2-((4,5-dimethylthiazol-2-yl)carbamoyl)phenyl)amino)-2,18-dioxo-6,9,12,15-tetraoxa-3-azaoctadecyl)piperazin-1-yl)pyridin-2-yl)amino)-N,N-dimethyl-7H-pyrrolo[2,3-d]pyrimidine-6-carboxamide (CPD-297)

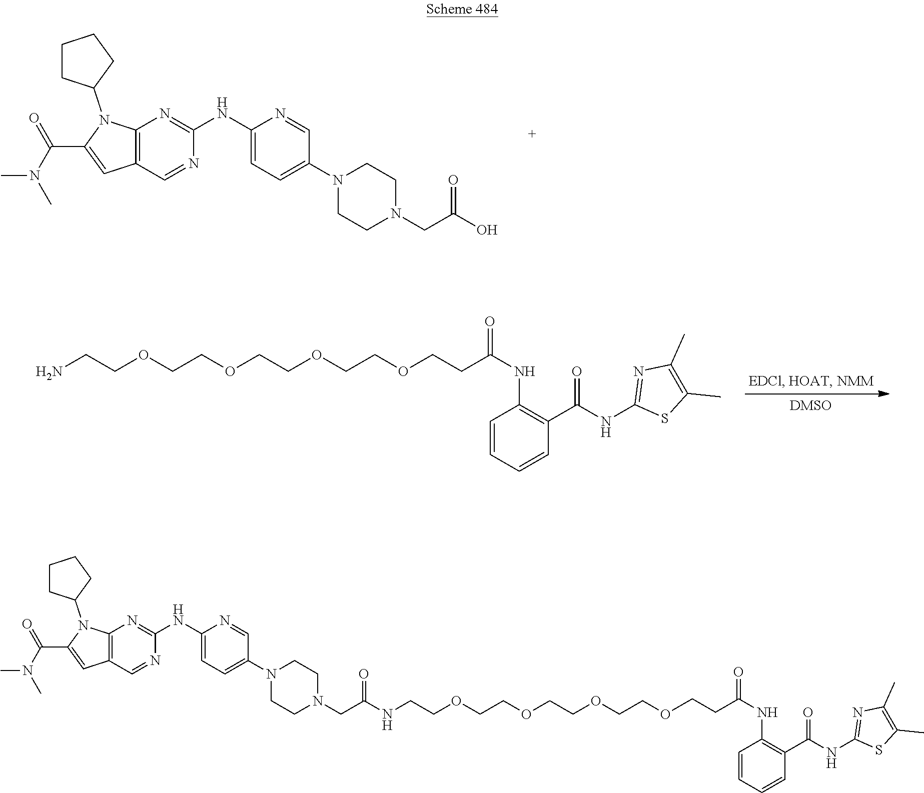

CPD-297 was synthesized following the standard procedure for preparing CPD-008 (4.5 mg, 31% yield) as a yellow solid. MS (ESI) m/z 969.5 [M+H]⁺.

### Example 485. 7-Cyclopentyl-2-((5-(4-(21-((2-((4,5-dimethylthiazol-2-yl)carbamoyl)phenyl)amino)-2,21-dioxo-6,9,12,15,18-pentaoxa-3-azahenicosyl)piperazin-1-yl)pyridin-2-yl)amino)-N,N-dimethyl-7H-pyrrolo[2,3-d]pyrimidine-6-carboxamide (CPD-298)

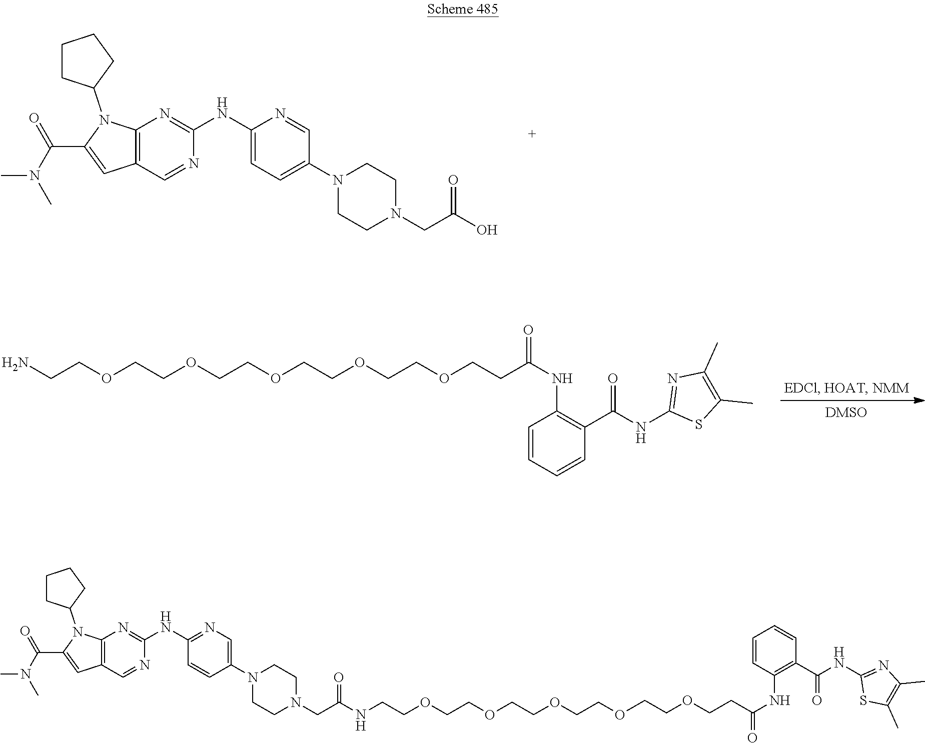

CPD-298 was synthesized following the standard procedure for preparing CPD-008 (3.5 mg, 23% yield) as a yellow solid. MS (ESI) m/z 1013.6 [M+H]⁺.

### Example 486. 7-Cyclopentyl-2-((5-(4-(24-((2-((4,5-dimethylthiazol-2-yl)carbamoyl)phenyl)amino)-2,24-dioxo-6,9,12,15,18,21-hexaoxa-3-azatetracosyl)piperazin-1-yl)pyridin-2-yl)amino)-N,N-dimethyl-7H-pyrrolo[2,3-d]pyrimidine-6-carboxamide (CPD-299)

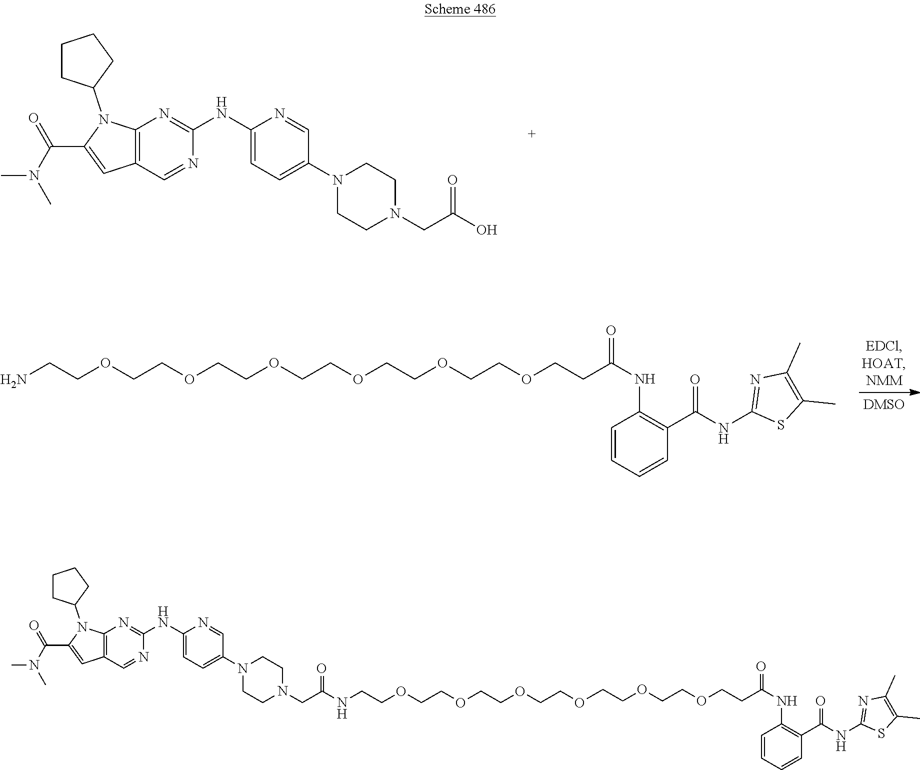

CPD-299 was synthesized following the standard procedure for preparing CPD-008 (4.2 mg, 26% yield) as a yellow solid. MS (ESI) m/z 1057.6 [M+H]⁺.

### Example 487. N-(4,5-Dimethylthiazol-2-yl)-2-(2-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)acetamido)benzamide (CPD-300)

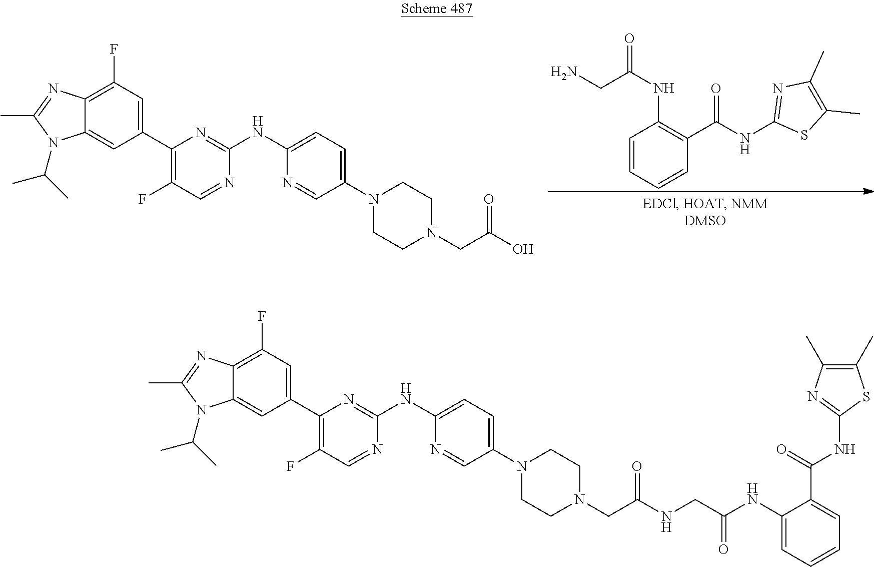

CPD-300 was synthesized following the standard procedure for preparing CPD-008 (7.4 mg, 41% yield) as a yellow solid. MS (ESI) m/z 809.4 [M+H]⁺.

### Example 488. N-(4,5-Dimethylthiazol-2-yl)-2-(3-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)propanamido)benzamide (CPD-301)

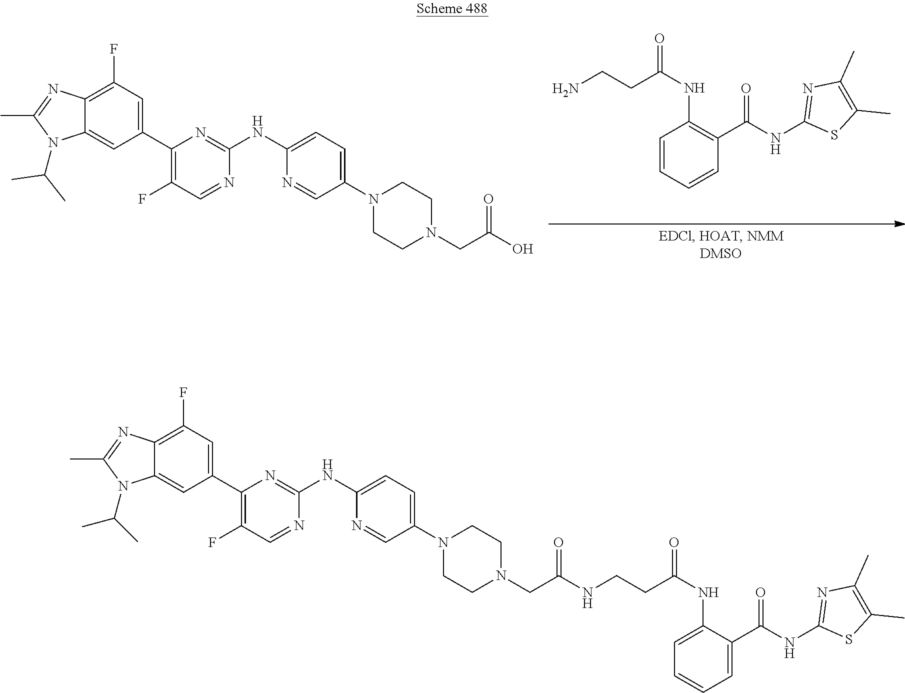

CPD-301 was synthesized following the standard procedure for preparing CPD-008 (4.9 mg, 27% yield) as a yellow solid. MS (ESI) m/z 823.4 [M+H]⁺.

### Example 489. N-(4,5-Dimethylthiazol-2-yl)-2-(4-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)butanamido)benzamide (CPD-302)

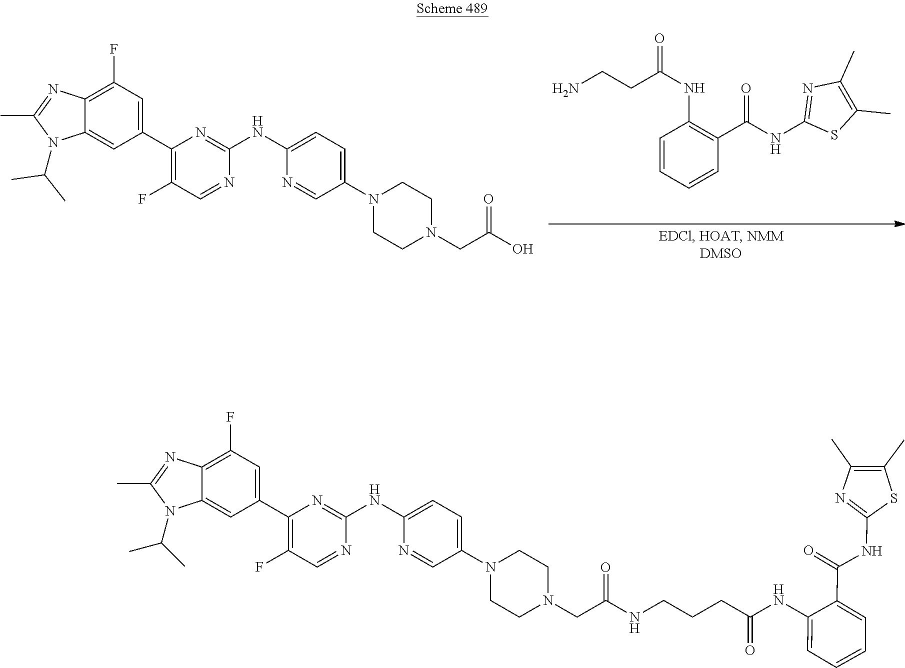

CPD-302 was synthesized following the standard procedure for preparing CPD-008 (3.8 mg, 20% yield) as a yellow solid. MS (ESI) m/z 837.4 [M+H]⁺.

### Example 490. N-(4,5-Dimethylthiazol-2-yl)-2-(5-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)pentanamido)benzamide (CPD-303)

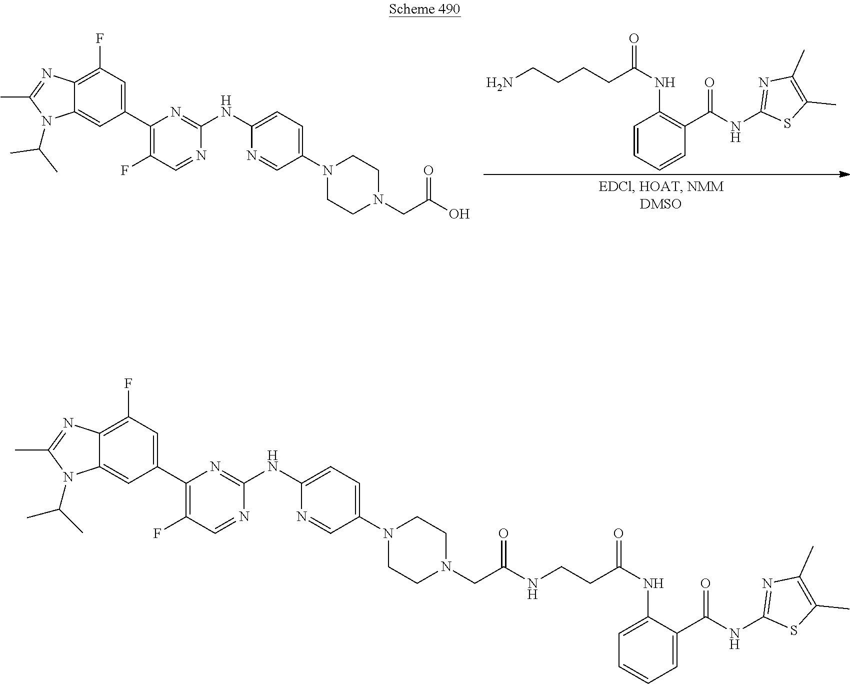

CPD-303 was synthesized following the standard procedure for preparing CPD-008 (2.5 mg, 13% yield) as a yellow solid. MS (ESI) m/z 831.4 [M+H]⁺.

### Example 491. N-(4,5-Dimethylthiazol-2-yl)-2-(6-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)hexanamido)benzamide (CPD-304)

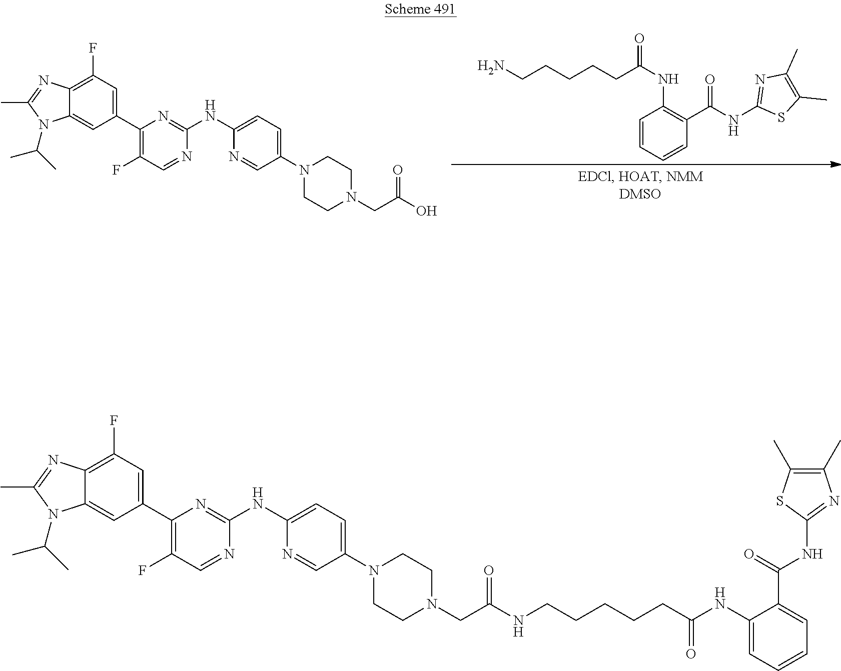

CPD-304 was synthesized following the standard procedure for preparing CPD-008 (2.9 mg, 15% yield) as a yellow solid. MS (ESI) m/z 865.4 [M+H]⁺.

### Example 492. N-(4,5-Dimethylthiazol-2-yl)-2-(7-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)heptanamido)benzamide (CPD-305)

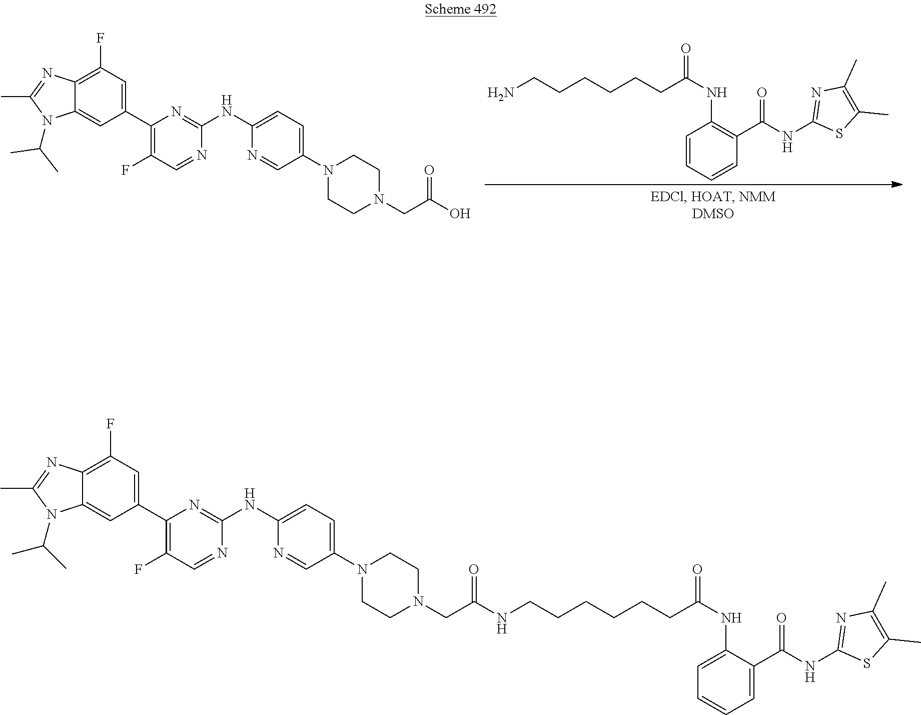

CPD-305 was synthesized following the standard procedure for preparing CPD-008 (2.3 mg, 12% yield) as a yellow solid. MS (ESI) m/z 879.4 [M+H]⁺.

### Example 493. N-(4,5-Dimethylthiazol-2-yl)-2-(8-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)octanamido)benzamide (CPD-306)

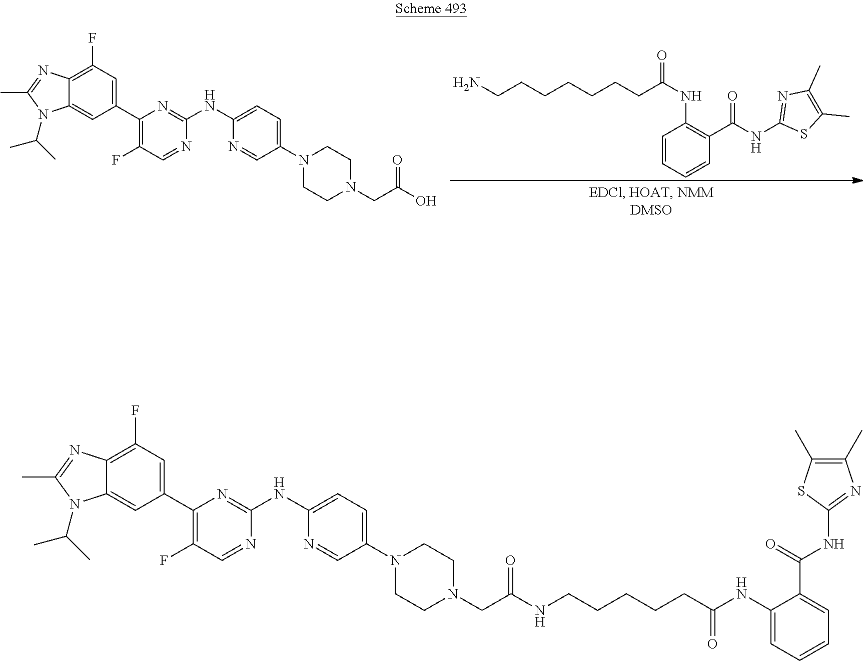

CPD-306 was synthesized following the standard procedure for preparing CPD-008 (2.5 mg, 12% yield) as a yellow solid. MS (ESI) m/z 893.4 [M+H]⁺.

### Example 494. N-(4,5-Dimethylthiazol-2-yl)-2-(9-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)nonanamido)benzamide (CPD-307)

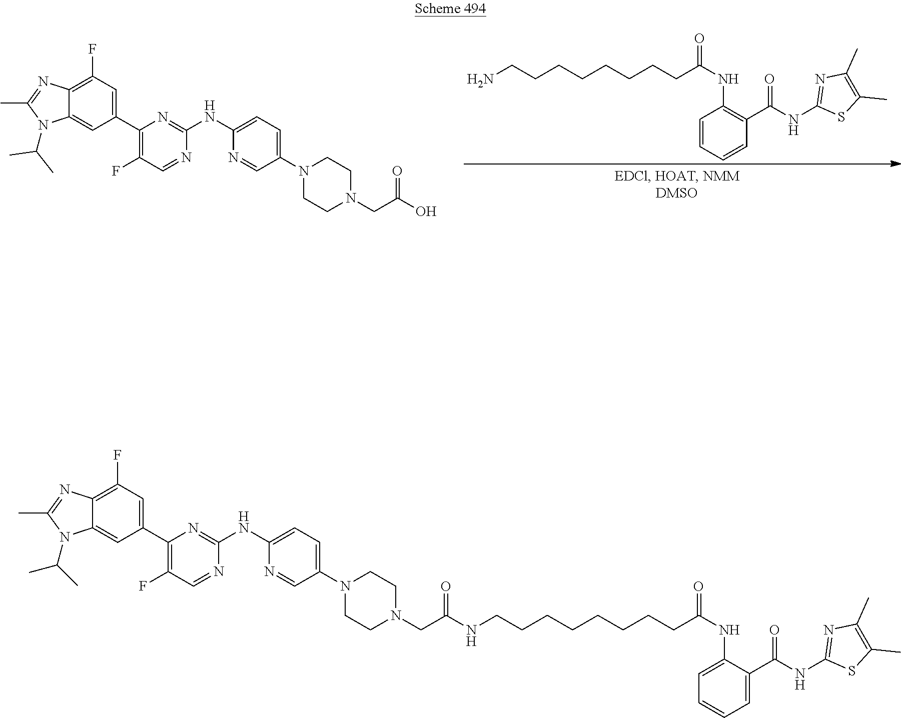

CPD-307 was synthesized following the standard procedure for preparing CPD-008 (0.9 mg, 5% yield) as a yellow solid. MS (ESI) m/z 907.5 [M+H]⁺.

### Example 495. N-(4,5-Dimethylthiazol-2-yl)-2-(3-(2-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)ethoxy)propanamido)benzamide (CPD-308)

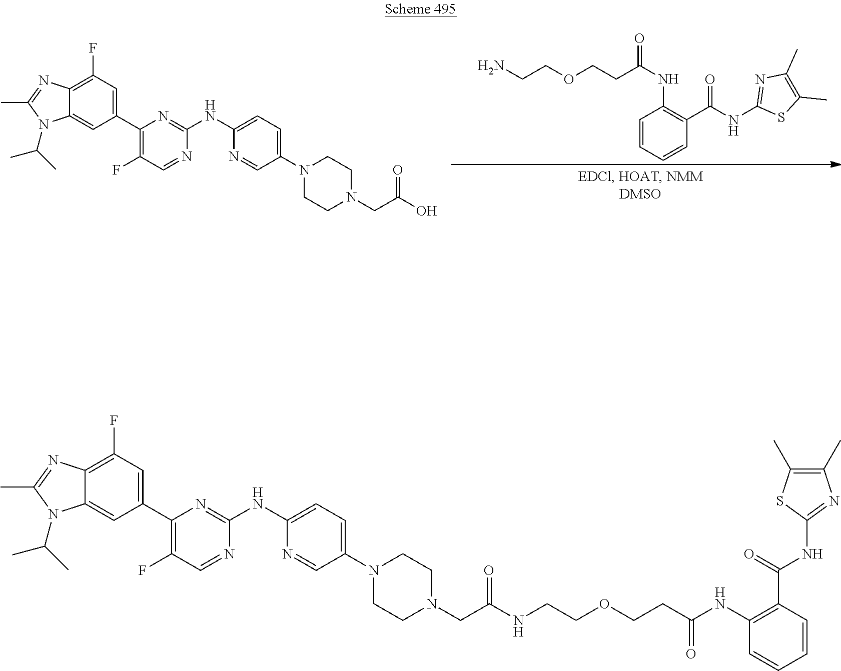

CPD-308 was synthesized following the standard procedure for preparing CPD-008 (8.0 mg, 42% yield) as a yellow solid. MS (ESI) m/z 867.4 [M+H]⁺.

### Example 496. N-(4,5-Dimethylthiazol-2-yl)-2-(3-(2-(2-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)ethoxy)ethoxy)propanamido)benzamide (CPD-309)

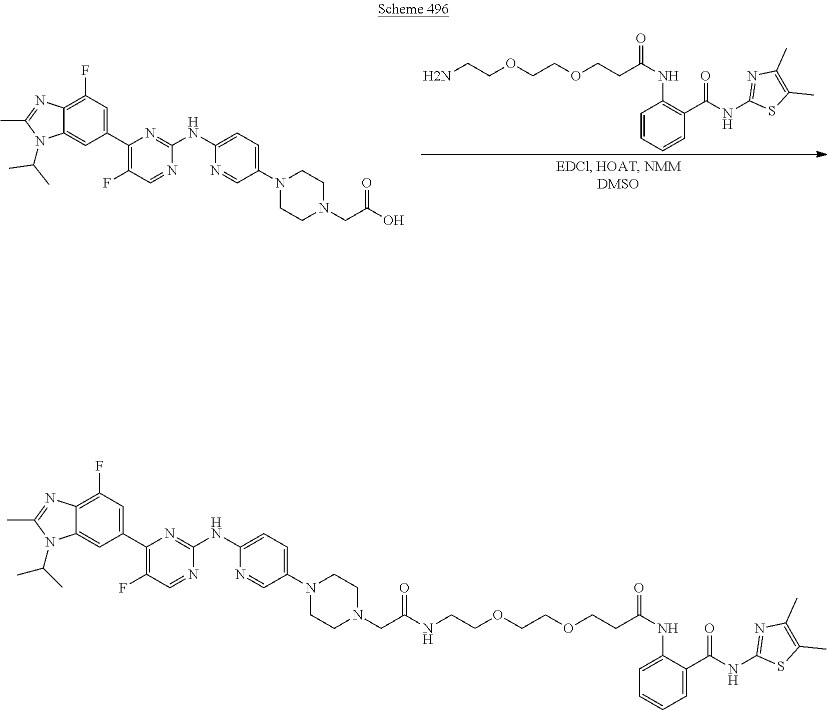

CPD-309 was synthesized following the standard procedure for preparing CPD-008 (7.1 mg, 36% yield) as a yellow solid. MS (ESI) m/z 911.4 [M+H]⁺.

### Example 497. N-(4,5-Dimethylthiazol-2-yl)-2-(1-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-2-oxo-6,9,12-trioxa-3-azapentadecan-15-amido)benzamide (CPD-310)

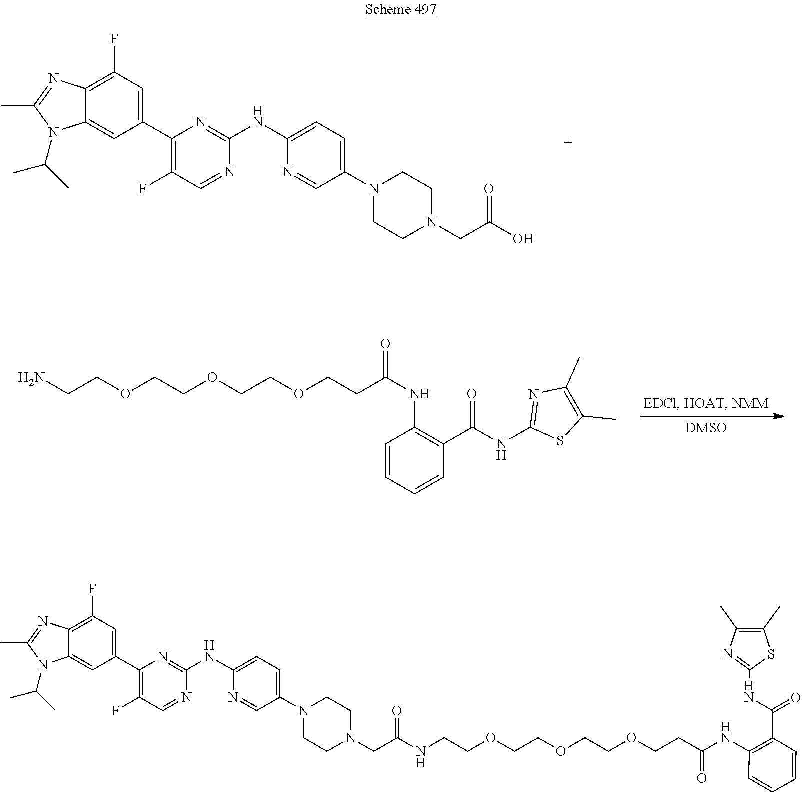

CPD-310 was synthesized following the standard procedure for preparing CPD-008 (4.3 mg, 21% yield) as a yellow solid. MS (ESI) m/z 955.5 [M+H]⁺.

### Example 498. N-(4,5-Dimethylthiazol-2-yl)-2-(1-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-2-oxo-6,9,12,15-tetraoxa-3-azaoctadecan-18-amido)benzamide (CPD-311)

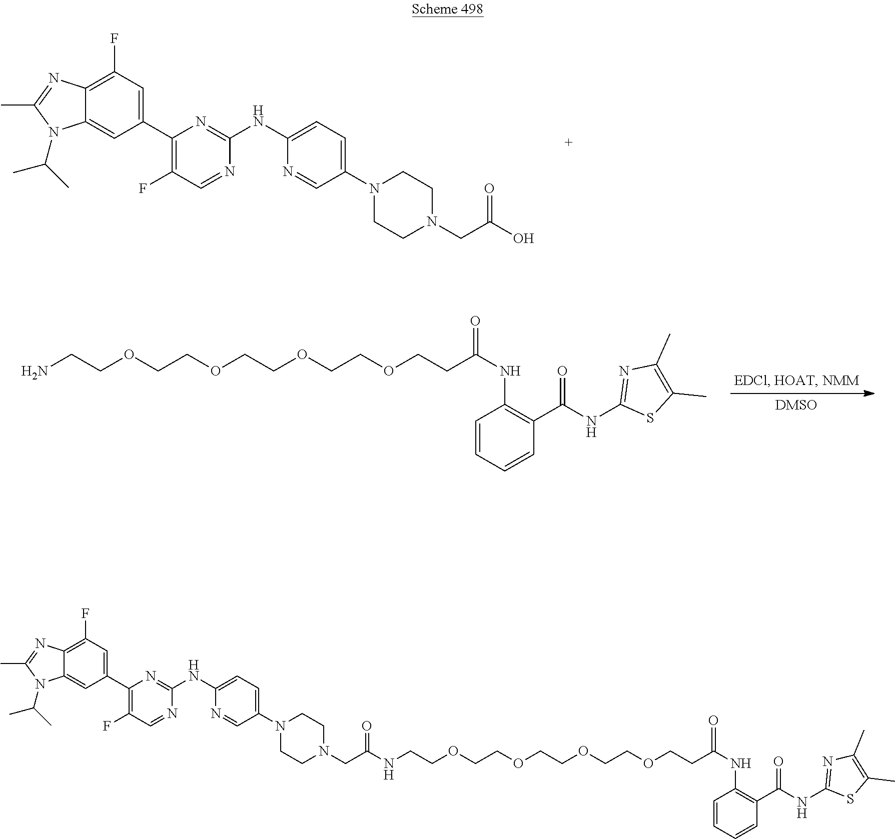

CPD-311 was synthesized following the standard procedure for preparing CPD-008 (3.8 mg, 17% yield) as a yellow solid. MS (ESI) m/z 999.5 [M+H]⁺.

### Example 499. N-(4,5-Dimethylthiazol-2-yl)-2-(1-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-2-oxo-6,9,12,15,18-pentaoxa-3-azahenicosan-21-amido)benzamide (CPD-312)

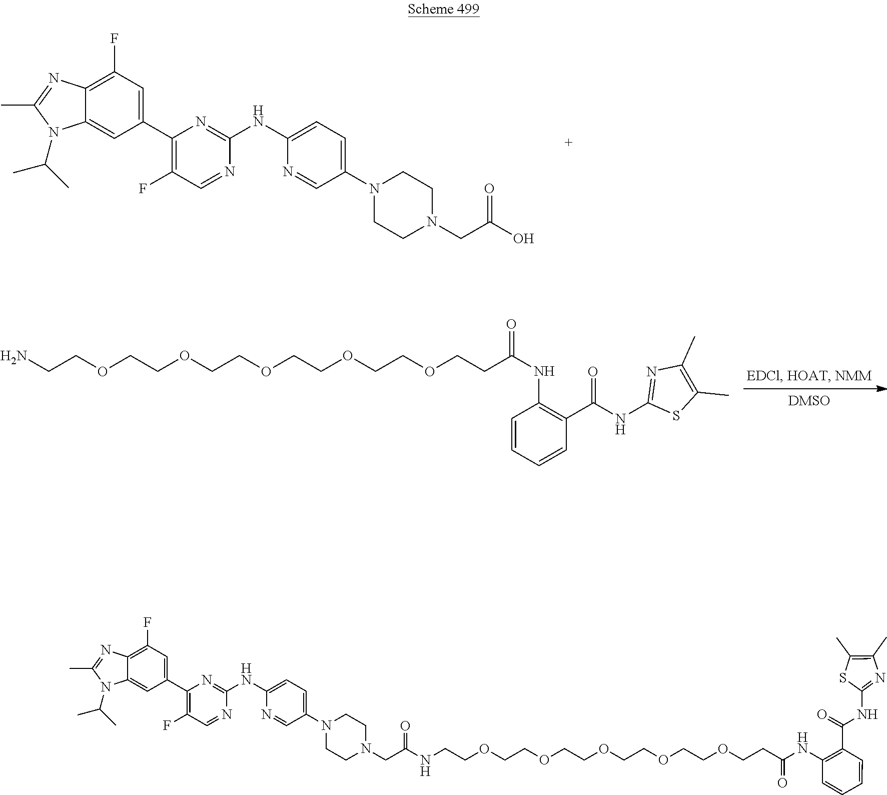

CPD-312 was synthesized following the standard procedure for preparing CPD-008 (5.1 mg, 23% yield) as a yellow solid. MS (ESI) m/z 1043.5 [M+H]⁺.

### Example 500. N-(4,5-Dimethylthiazol-2-yl)-2-(1-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-2-oxo-6,9,12,15,18,21-hexaoxa-3-azatetracosan-24-amido)benzamide (CPD-313)

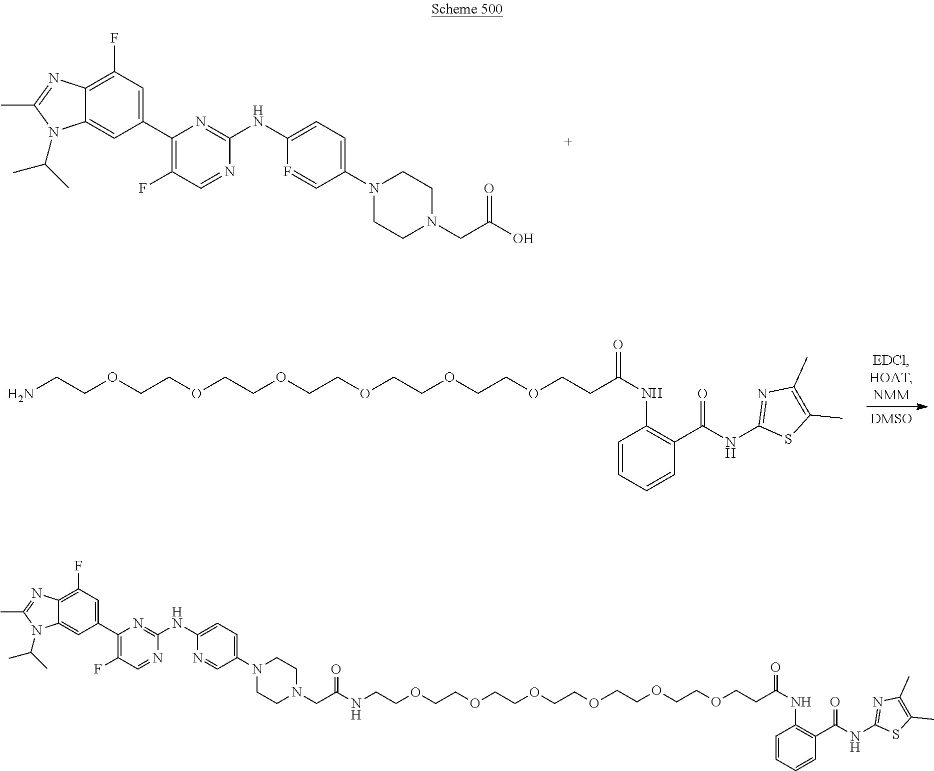

CPD-313 was synthesized following the standard procedure for preparing CPD-008 (1.8 mg, 7% yield) as a yellow solid. MS (ESI) m/z 1087.5 [M+H]⁺.

### Example 501. N-(4,5-Dimethylthiazol-2-yl)-2-(2-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)acetamido)benzamide (CPD-314)

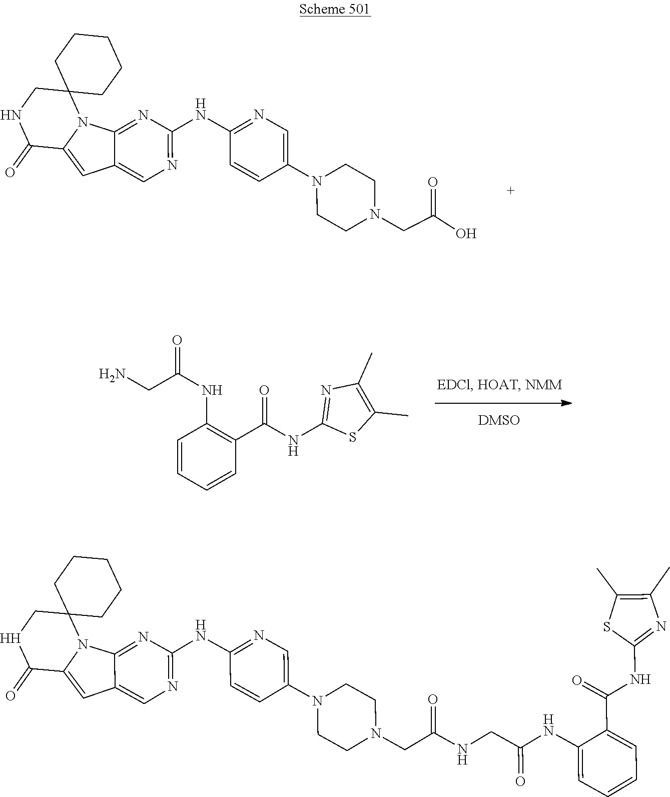

CPD-314 was synthesized following the standard procedure for preparing CPD-008 (4.2 mg, 23% yield) as a yellow solid. MS (ESI) m/z 777.4 [M+H]⁺.

### Example 502. N-(4,5-Dimethylthiazol-2-yl)-2-(3-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)propanamido)benzamide (CPD-315)

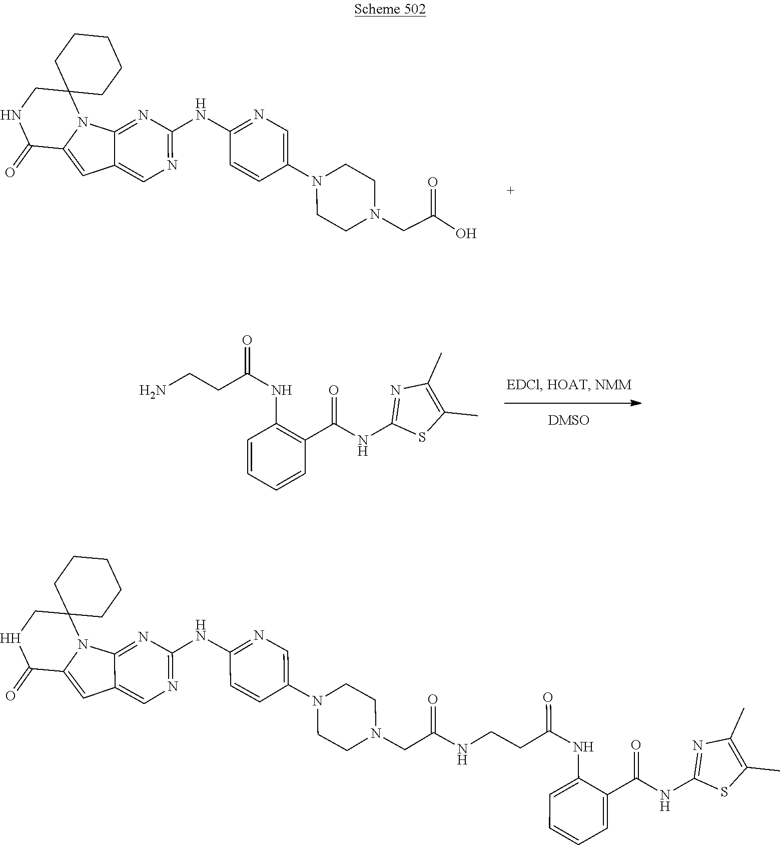

CPD-315 was synthesized following the standard procedure for preparing CPD-008 (1.8 mg, 9% yield) as a yellow solid. MS (ESI) m/z 791.4 [M+H]⁺.

### Example 503. N-(4,5-Dimethylthiazol-2-yl)-2-(4-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)butanamido)benzamide (CPD-316)

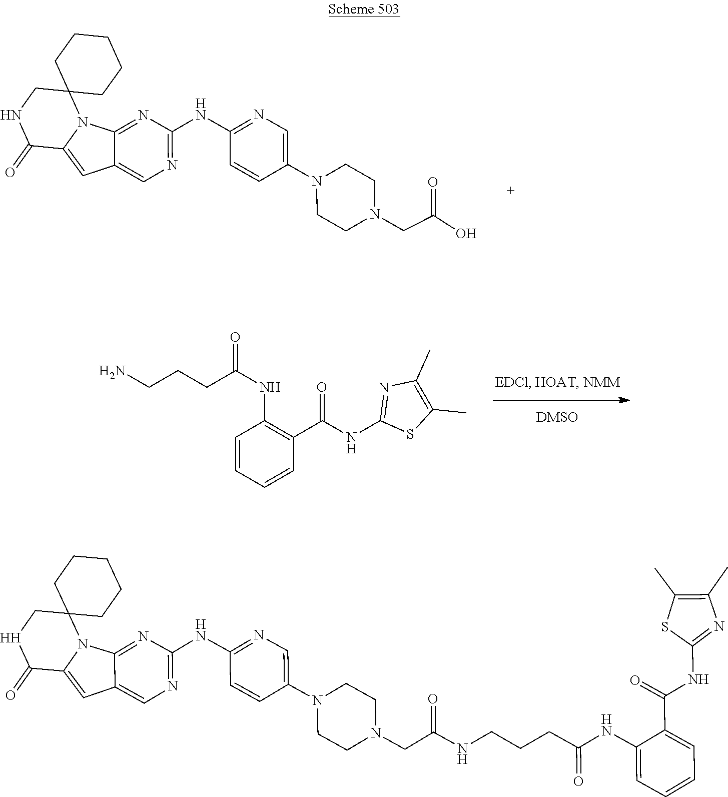

CPD-316 was synthesized following the standard procedure for preparing CPD-008 (1.5 mg, 8% yield) as a yellow solid. MS (ESI) m/z 805.3 [M+H]⁺.

### Example 504. N-(4,5-Dimethylthiazol-2-yl)-2-(5-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)pentanamido)benzamide (CPD-317)

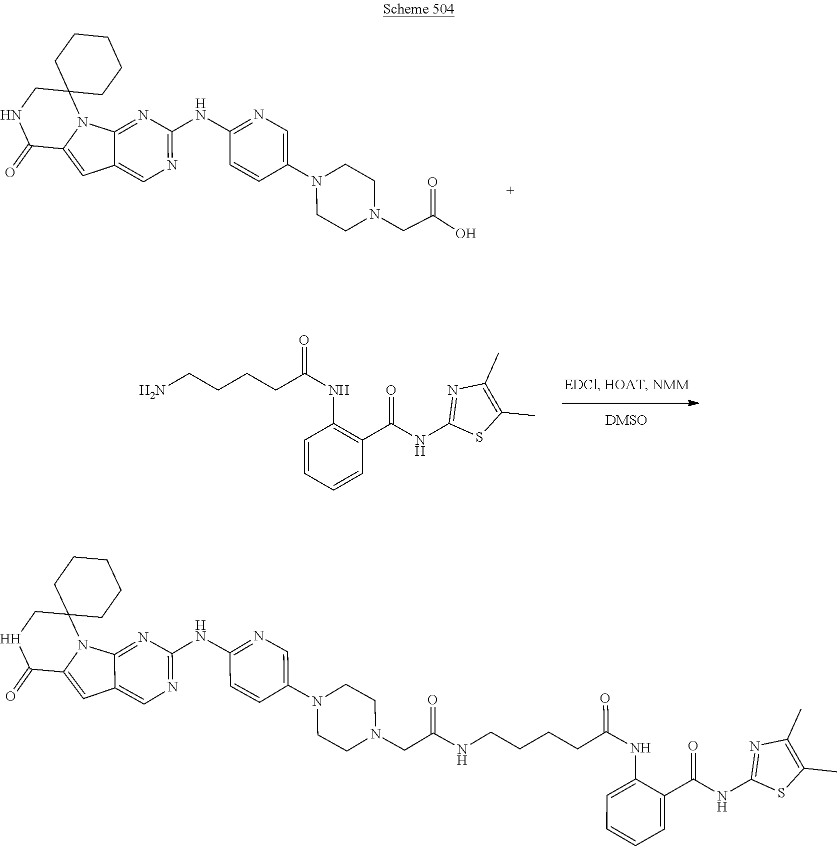

CPD-317 was synthesized following the standard procedure for preparing CPD-008 (1.0 mg, 5% yield) as a yellow solid. MS (ESI) m/z 819.4 [M+H]⁺.

### Example 505. N-(4,5-Dimethylthiazol-2-yl)-2-(6-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)hexanamido)benzamide (CPD-318)

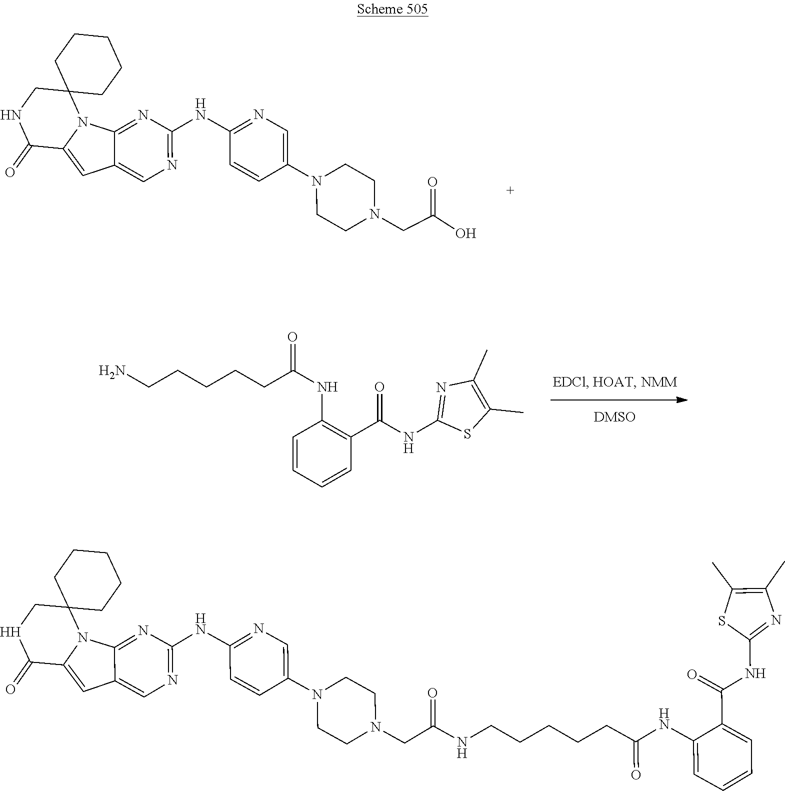

CPD-318 was synthesized following the standard procedure for preparing CPD-008 (1.1 mg, 5% yield) as a yellow solid. MS (ESI) m/z 833.4 [M+H]⁺.

### Example 506. N-(4,5-Dimethylthiazol-2-yl)-2-(7-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)heptanamido)benzamide (CPD-319)

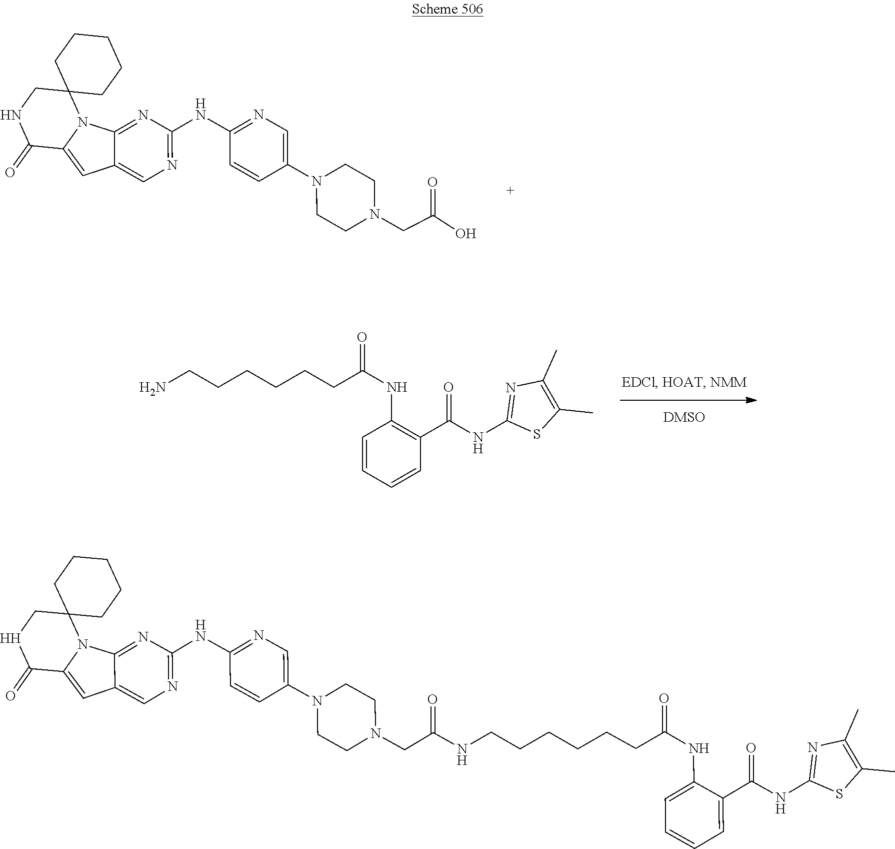

CPD-319 was synthesized following the standard procedure for preparing CPD-008 (0.5 mg, 2% yield) as a yellow solid. MS (ESI) m/z 847.4 [M+H]⁺.

### Example 507. N-(4,5-Dimethylthiazol-2-yl)-2-(8-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)octanamido)benzamide (CPD-320)

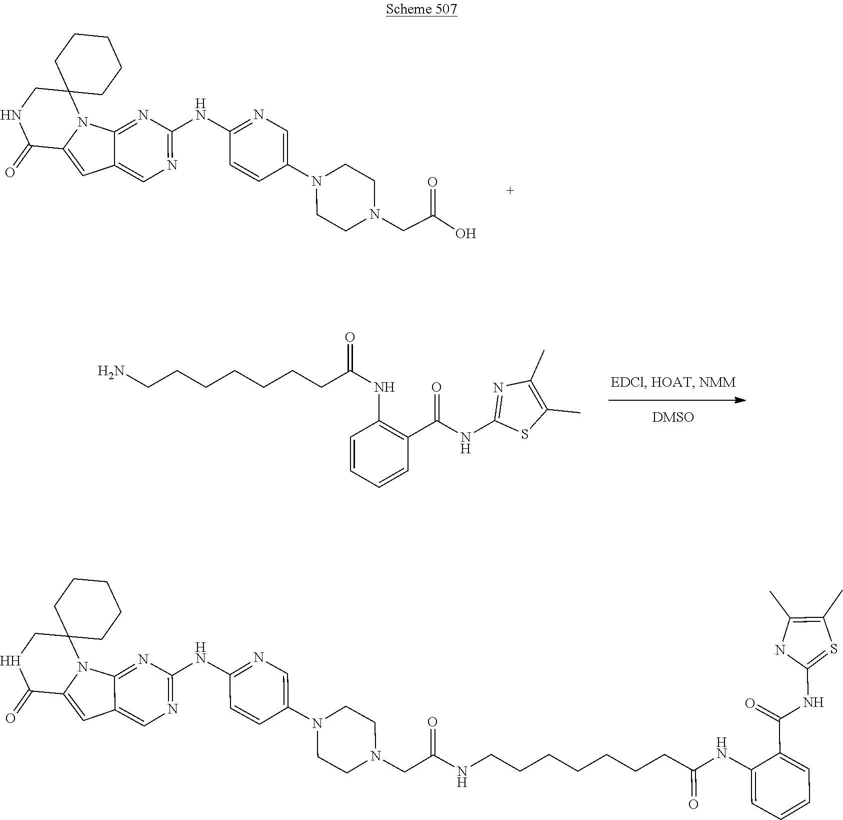

CPD-320 was synthesized following the standard procedure for preparing CPD-008 (1.2 mg, 6% yield) as a yellow solid. MS (ESI) m/z 861.4 [M+H]⁺.

### Example 508. N-(4,5-Dimethylthiazol-2-yl)-2-(9-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)nonanamido)benzamide (CPD-321)

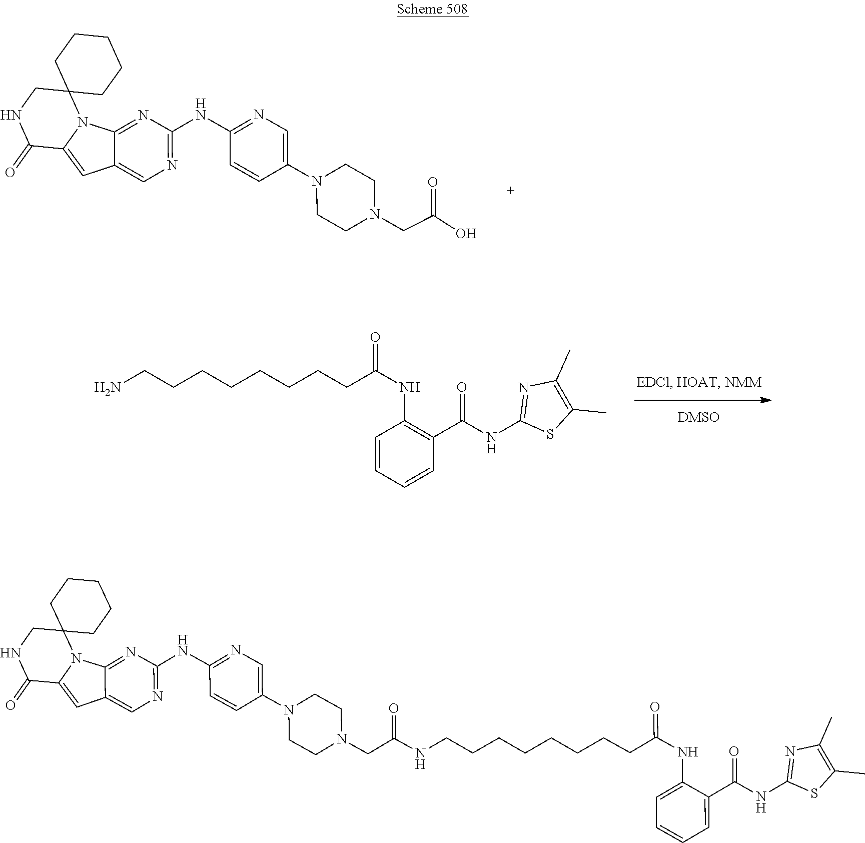 [SMILES: O=C(O)CN1CCN(c2ccc(Nc3ncc4cc5n(c4n3)C3(CCCCC3)CNC5=O)nc2)CC1 ; Cc1nc(NC(=O)c2ccccc2NC(=O)CCCCCCCCN)sc1C ; Cc1nc(NC(=O)c2ccccc2NC(=O)CCCCCCCCNC(=O)CN2CCN(c3ccc(Nc4ncc5cc6n(c5n4)C4(CCCCC4)CNC6=O)nc3)CC2)sc1C]

CPD-321 was synthesized following the standard procedure for preparing CPD-008 (1.0 mg, 5% yield) as a yellow solid. MS (ESI) m/z 875.5 [M+H]⁺.

### Example 509. N-(4,5-Dimethylthiazol-2-yl)-2-(3-(2-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)ethoxy)propanamido)benzamide (CPD-322)

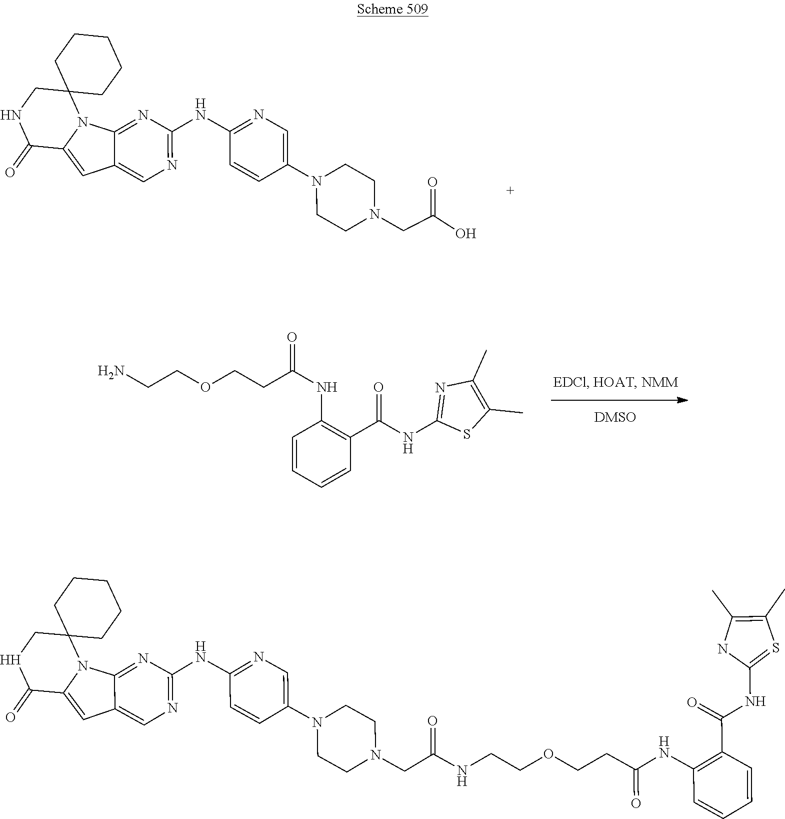

CPD-322 was synthesized following the standard procedure for preparing CPD-008 (6.4 mg, 33% yield) as a yellow solid. MS (ESI) m/z 835.4 [M+H]⁺.

### Example 510. N-(4,5-Dimethylthiazol-2-yl)-2-(3-(2-(2-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)ethoxy)ethoxy)propanamido)benzamide (CPD-323)

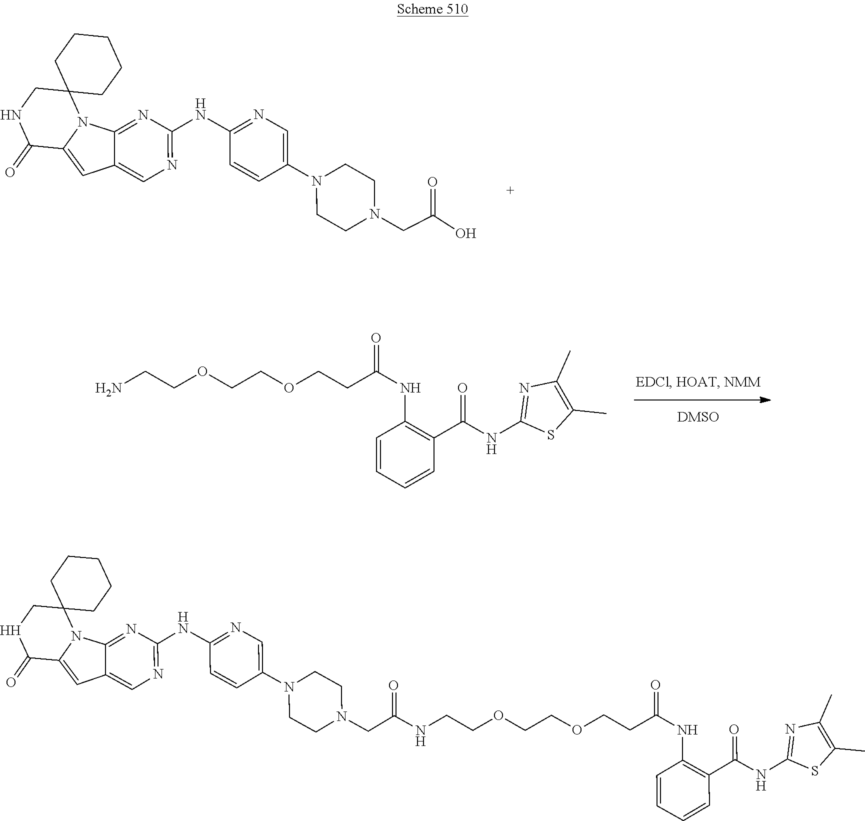

CPD-323 was synthesized following the standard procedure for preparing CPD-008 (5.4 mg, 26% yield) as a yellow solid. MS (ESI) m/z 879.4 [M+H]⁺.

### Example 511. N-(4,5-Dimethylthiazol-2-yl)-2-(2-oxo-1-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)-6,9,12-trioxa-3-azapentadecan-15-amido)benzamide (CPD-324)

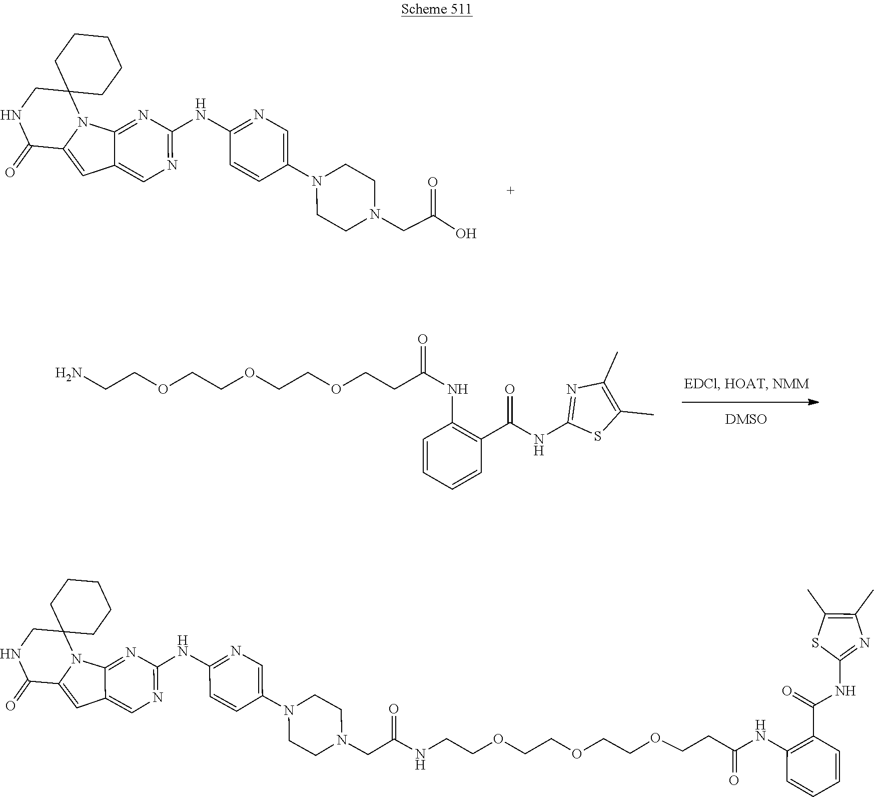 [SMILES: O=C(O)CN1CCN(c2ccc(Nc3ncc4cc5n(c4n3)C3(CCCCC3)CNC5=O)nc2)CC1 ; Cc1nc(NC(=O)c2ccccc2NC(=O)CCOCCOCCOCCN)sc1C ; Cc1nc(NC(=O)c2ccccc2NC(=O)CCOCCOCCOCCNC(=O)CN2CCN(c3ccc(Nc4ncc5cc6n(c5n4)C4(CCCCC4)CNC6=O)nc3)CC2)sc1C]

CPD-324 was synthesized following the standard procedure for preparing CPD-008 (4.0 mg, 18% yield) as a yellow solid. MS (ESI) m/z 923.5 [M+H]⁺.

### Example 512. N-(4,5-Dimethylthiazol-2-yl)-2-(2-oxo-1-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)-6,9,12,15-tetraoxa-3-azaoctadecan-18-amido)benzamide (CPD-325)

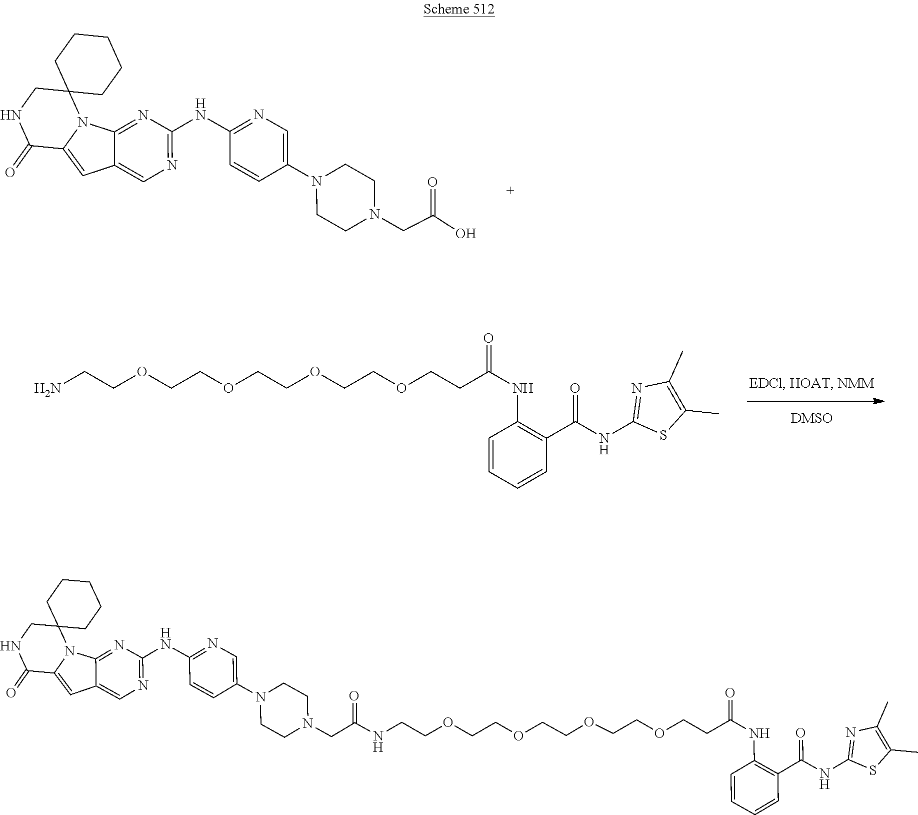 [SMILES: O=C(O)CN1CCN(c2ccc(Nc3ncc4cc5n(c4n3)C3(CCCCC3)CNC5=O)nc2)CC1 ; Cc1nc(NC(=O)c2ccccc2NC(=O)CCOCCOCCOCCOCCN)sc1C ; Cc1nc(NC(=O)c2ccccc2NC(=O)CCOCCOCCOCCOCCNC(=O)CN2CCN(c3ccc(Nc4ncc5cc6n(c5n4)C4(CCCCC4)CNC6=O)nc3)CC2)sc1C]

CPD-325 was synthesized following the standard procedure for preparing CPD-008 (3.6 mg, 16% yield) as a yellow solid. MS (ESI) m/z 967.5 [M+H]⁺.

### Example 513. N-(4,5-Dimethylthiazol-2-yl)-2-(2-oxo-1-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)-6,9,12,15,18-pentaoxa-3-azahenicosan-21-amido)benzamide (CPD-326)

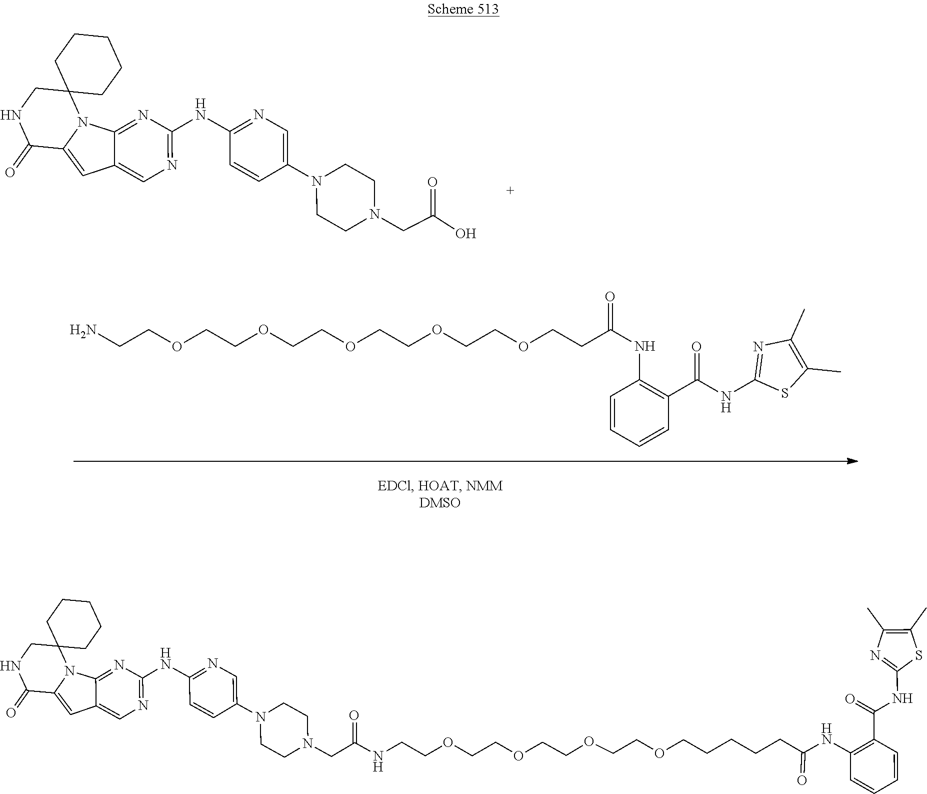 [SMILES: O=C(O)CN1CCN(c2ccc(Nc3ncc4cc5n(c4n3)C3(CCCCC3)CNC5=O)nc2)CC1 ; Cc1nc(NC(=O)c2ccccc2NC(=O)CCOCCOCCOCCOCCOCCN)sc1C ; Cc1nc(NC(=O)c2ccccc2NC(=O)CCCCCOCCOCCOCCOCCNC(=O)CN2CCN(c3ccc(Nc4ncc5cc6n(c5n4)C4(CCCCC4)CNC6=O)nc3)CC2)sc1C]

CPD-326 was synthesized following the standard procedure for preparing CPD-008 (4.1 mg, 17% yield) as a yellow solid. MS (ESI) m/z 1011.5 [M+H]⁺.

### Example 514. N-(4,5-Dimethylthiazol-2-yl)-2-(2-oxo-1-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)-6,9,12,15,18,21-hexaoxa-3-azatetracosan-24-amido)benzamide (CPD-327)

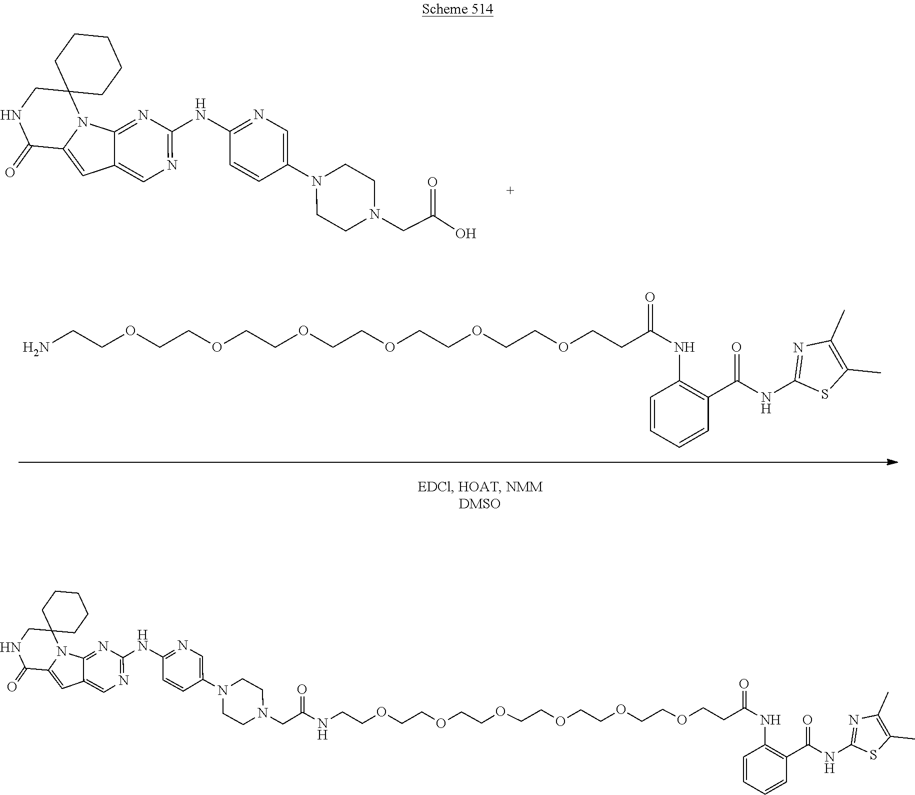 [SMILES: O=C(O)CN1CCN(c2ccc(Nc3ncc4cc5n(c4n3)C3(CCCCC3)CNC5=O)nc2)CC1 ; Cc1nc(NC(=O)c2ccccc2NC(=O)CCOCCOCCOCCOCCOCCOCCN)sc1C ; Cc1nc(NC(=O)c2ccccc2NC(=O)CCOCCOCCOCCOCCOCCOCCNC(=O)CN2CCN(c3ccc(Nc4ncc5cc6n(c5n4)C4(CCCCC4)CNC6=O)nc3)CC2)sc1C]

CPD-327 was synthesized following the standard procedure for preparing CPD-008 (2.8 mg, 11% yield) as a yellow solid. MS (ESI) m/z=1055.6 [M+H]⁺.

### Example 515. 3-((7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-2-methyl-N-(6-methylpyridin-3-yl)benzamide (CPD-328)

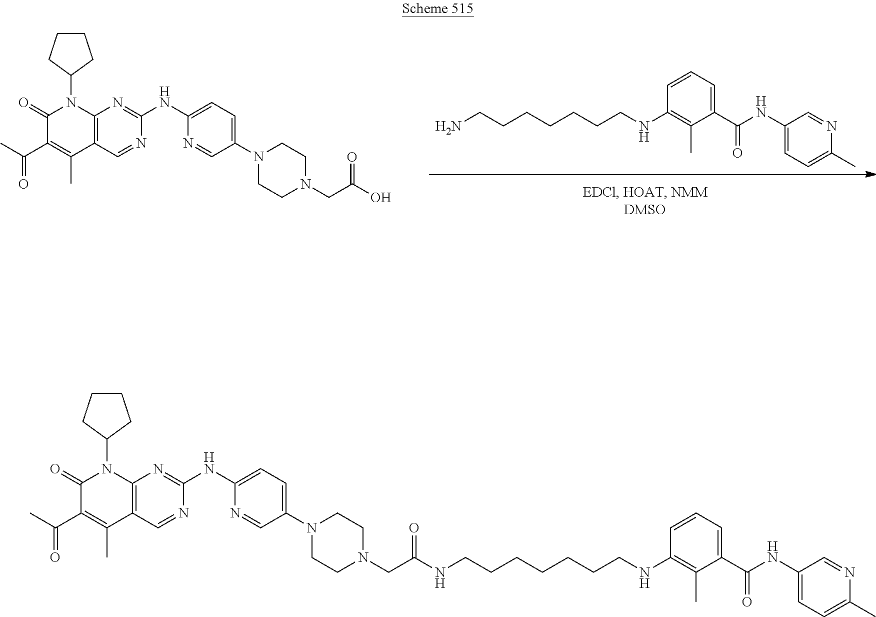 [SMILES: CC(=O)c1c(C)c2cnc(Nc3ccc(N4CCN(CC(=O)O)CC4)cn3)nc2n(C2CCCC2)c1=O ; Cc1ccc(NC(=O)c2cccc(NCCCCCCCN)c2C)cn1 ; CC(=O)c1c(C)c2cnc(Nc3ccc(N4CCN(CC(=O)NCCCCCCCNc5cccc(C(=O)Nc6ccc(C)nc6)c5C)CC4)cn3)nc2n(C2CCCC2)c1=O]

CPD-328 was synthesized following the standard procedure for preparing CPD-008 (3.9 mg, 21% yield) as a yellow solid. MS (ESI) m/z 5842.9 [M+H]⁺.

### Example 516. 3-((7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(1,5-dimethyl-1H-pyrazol-3-yl)-2-methylbenzamide (CPD-329)

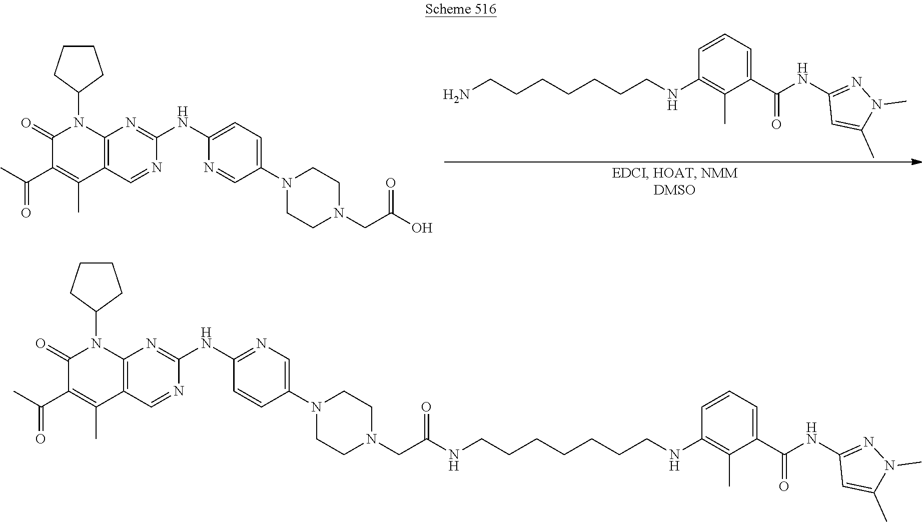 [SMILES: CC(=O)c1c(C)c2cnc(Nc3ccc(N4CCN(CC(=O)O)CC4)cn3)nc2n(C2CCCC2)c1=O ; Cc1c(NCCCCCCCN)cccc1C(=O)Nc1cc(C)n(C)n1 ; CC(=O)c1c(C)c2cnc(Nc3ccc(N4CCN(CC(=O)NCCCCCCCNc5cccc(C(=O)Nc6cc(C)n(C)n6)c5C)CC4)cn3)nc2n(C2CCCC2)c1=O]

CPD-329 was synthesized following the standard procedure for preparing CPD-008 (2.9 mg, 17% yield) as a yellow solid. MS (ESI) m/z 845.9 [M+H]⁺.

### Example 517. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(5-methylpyridin-2-v yl)benzamide (CPD-330)

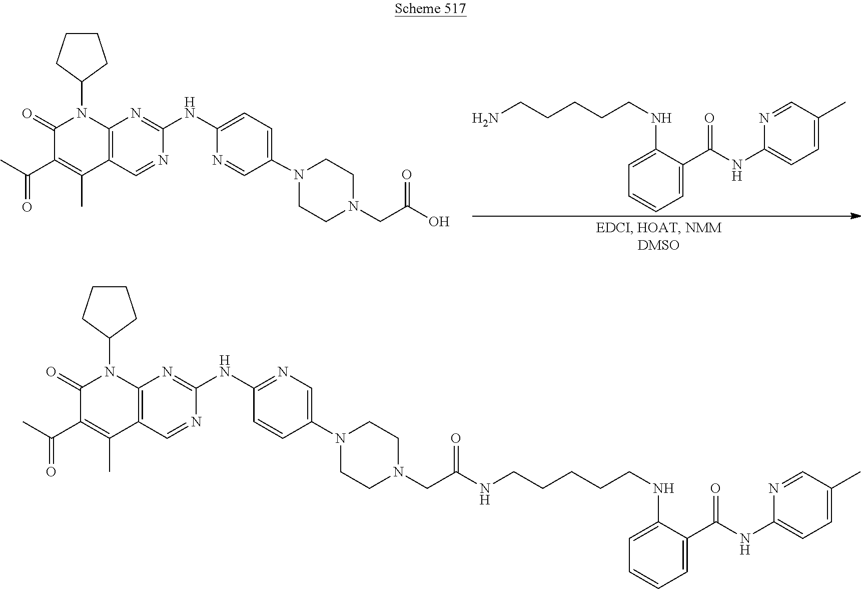 [SMILES: CC(=O)c1c(C)c2cnc(Nc3ccc(N4CCN(CC(=O)O)CC4)cn3)nc2n(C2CCCC2)c1=O ; Cc1ccc(NC(=O)c2ccccc2NCCCCCN)nc1 ; CC(=O)c1c(C)c2cnc(Nc3ccc(N4CCN(CC(=O)NCCCCCNc5ccccc5C(=O)Nc5ccc(C)cn5)CC4)cn3)nc2n(C2CCCC2)c1=O]

CPD-330 was synthesized following the standard procedure for preparing CPD-008 (15.9 mg, 67% yield) as a yellow solid. MS (ESI) m/z 800.8 [M+H]⁺.

### Example 518. 3-((7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-2-methyl-N-(6-methylpyridazin-3-yl)benzamide (CPD-331)

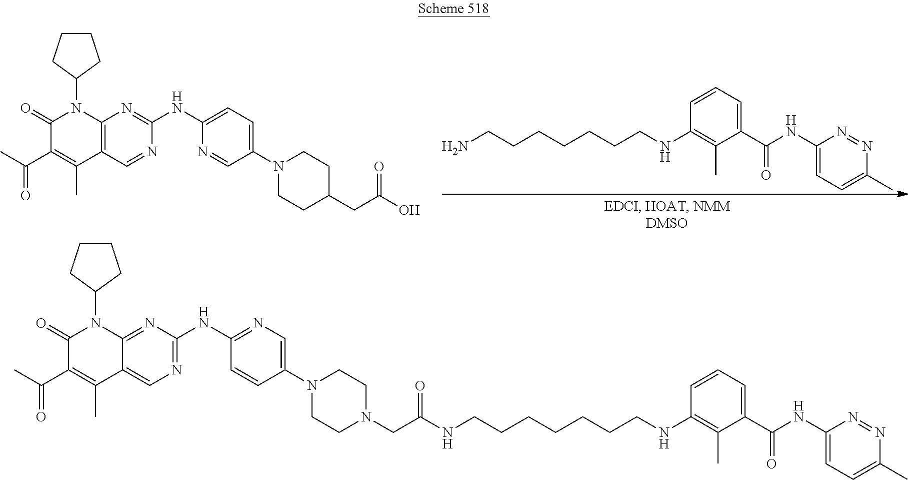 [SMILES: CC(=O)c1c(C)c2cnc(Nc3ccc(N4CCC(CC(=O)O)CC4)cn3)nc2n(C2CCCC2)c1=O ; Cc1ccc(NC(=O)c2cccc(NCCCCCCCN)c2C)nn1 ; CC(=O)c1c(C)c2cnc(Nc3ccc(N4CCN(CC(=O)NCCCCCCCNc5cccc(C(=O)Nc6ccc(C)nn6)c5C)CC4)cn3)nc2n(C2CCCC2)c1=O]

CPD-331 was synthesized following the standard procedure for preparing CPD-008 (4.6 mg, 21% yield) as a yellow solid. MS (ESI) m/z 843.8 [M+H]⁺.

### Example 519. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-N-methylacetamido)ethoxy)ethoxy)propanamido)-N-(6-methoxypyridazin-3-yl)benzamide (CPD-332)

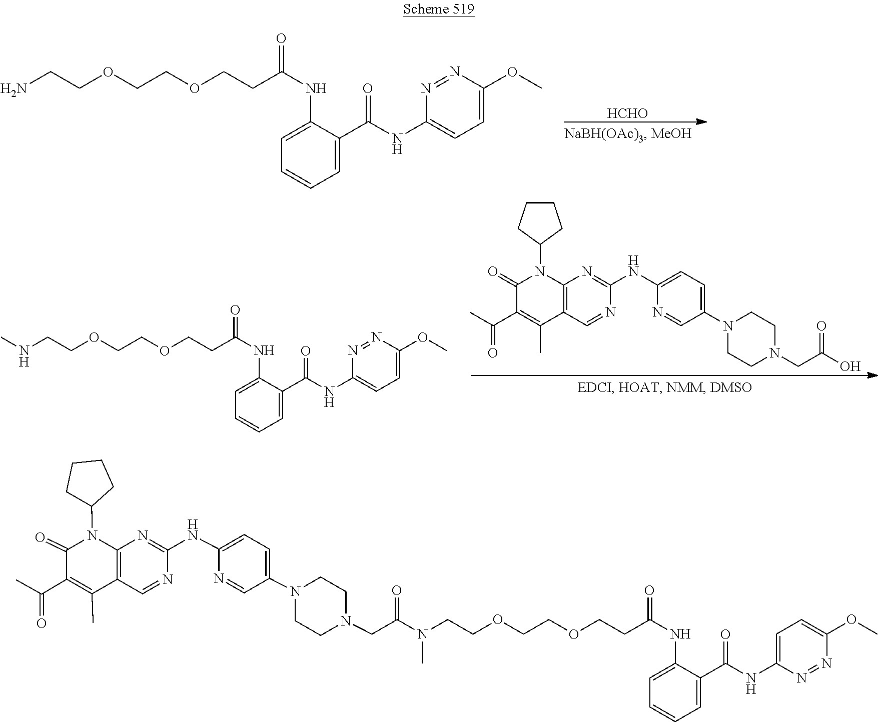

### Step 1. Synthesis of N-(6-methoxypyridazin-3-yl)-2-(3-(2-(2-(methylamino)ethoxy)ethoxy)propanamido)benzamide

To a solution of 2-(3-(2-(2-aminoethoxy)ethoxy)propanamido)-N-(6-methoxypyridazin-3-yl)benzamide (50 mg, 0.12 mol) in MeOH (10 mL) were added NaBH(OAc)₃ (52.5 mg, 0.24 mmol) and HCHO (5.07 mg, 0.14 mmol) at 0° C. After the reaction mixture was stirred at rt for 1 h, it was concentrated and purified by prep-TLC to provide the desired product (5.1 mg, 9% yield) as a white solid. MS (ESI) m/z=418.4 [M+H]⁺.

### Step 2. Synthesis of 2-(3-(2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-methylacetamido)ethoxy)ethoxy)propanamido)-N-(6-methoxypyridazin-3-yl)benzamide

The title compound was synthesized following the standard procedure for preparing CPD-008 (3.2 mg, 28% yield) as a yellow solid. MS (ESI) m/z 905.8 [M+H]⁺.

### Example 520. 3-((7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-2-methyl-N-(5-methylpyridin-2-yl)benzamide (CPD-333)

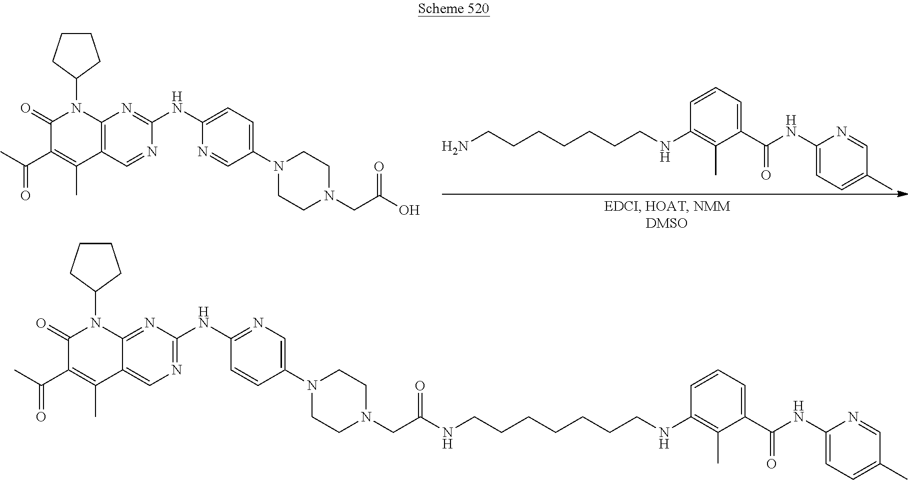 [SMILES: CC(=O)c1c(C)c2cnc(Nc3ccc(N4CCN(CC(=O)O)CC4)cn3)nc2n(C2CCCC2)c1=O ; Cc1ccc(NC(=O)c2cccc(NCCCCCCCN)c2C)nc1 ; CC(=O)c1c(C)c2cnc(Nc3ccc(N4CCN(CC(=O)NCCCCCCCNc5cccc(C(=O)Nc6ccc(C)cn6)c5C)CC4)cn3)nc2n(C2CCCC2)c1=O]

CPD-333 was synthesized following the standard procedure for preparing CPD-008 (6.8 mg, 37% yield) as a yellow solid. MS (ESI) m/z 842.5 [M+H]⁺.

### Example 521. 3-((7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(6-methoxypyridazin-3-yl)-2-methylbenzamide (CPD-334)

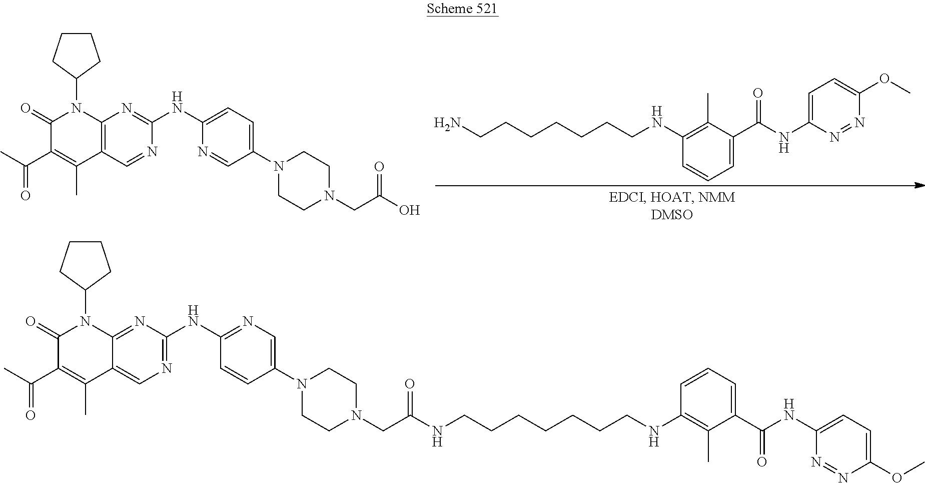 [SMILES: CC(=O)c1c(C)c2cnc(Nc3ccc(N4CCN(CC(=O)O)CC4)cn3)nc2n(C2CCCC2)c1=O ; COc1ccc(NC(=O)c2cccc(NCCCCCCCN)c2C)nn1 ; COc1ccc(NC(=O)c2cccc(NCCCCCCCNC(=O)CN3CCN(c4ccc(Nc5ncc6c(C)c(C(C)=O)c(=O)n(C7CCCC7)c6n5)nc4)CC3)c2C)nn1]

CPD-334 was synthesized following the standard procedure for preparing CPD-008 (13 mg, 64% yield) as a yellow solid. MS (ESI) m/z 859.9 [M+H]⁺.

### Example 522. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(6-(methylamino)pyridazin-3-yl)benzamide (CPD-335)

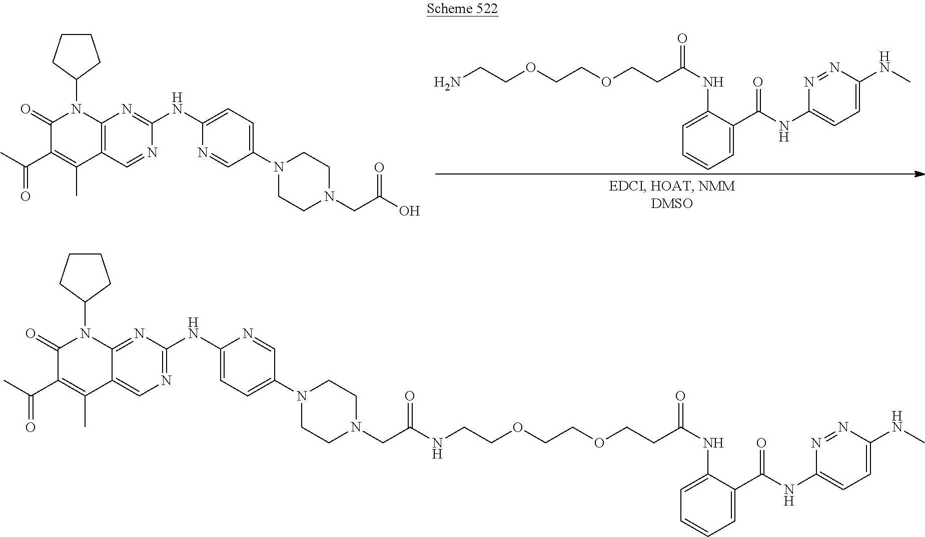 [SMILES: CC(=O)c1c(C)c2cnc(Nc3ccc(N4CCN(CC(=O)O)CC4)cn3)nc2n(C2CCCC2)c1=O ; CNc1ccc(NC(=O)c2ccccc2NC(=O)CCOCCOCCN)nn1 ; CNc1ccc(NC(=O)c2ccccc2NC(=O)CCOCCOCCNC(=O)CN2CCN(c3ccc(Nc4ncc5c(C)c(C(C)=O)c(=O)n(C6CCCC6)c5n4)nc3)CC2)nn1]

CPD-335 was synthesized following the standard procedure for preparing CPD-008 (7.1 mg, 27% yield) as a yellow solid. MS (ESI) m/z 890.8 [M+H]⁺.

### Example 523. 3-((7-Aminoheptyl)amino)-2-methyl-N-(6-methylpyridin-3-yl)benzamide (BL1-200)

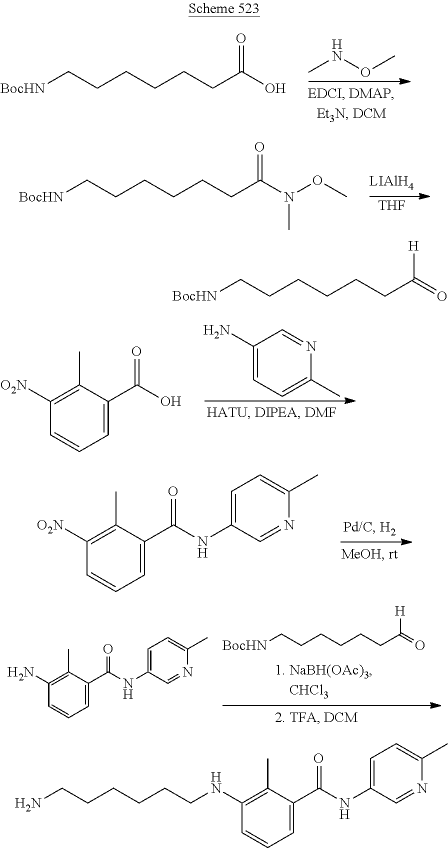

### Step 1. Synthesis of tert-butyl (7-(methoxy(methyl)amino)-7-oxoheptyl)carbamate

To a solution of 7-(tert-butoxycarbonylamino)heptanoic acid (2.6 g, 10 mmol) in DCM (30 mL) were added EDCI (2.9 g, 15 mmol), triethylamine (2.0 g, 20 mmol), N,O-dimethylhydroxylamine hydrochloride (2.9 g, 15 mmol) and DAMP (0.22 g, 1.0 mmol). After stirring at rt overnight, the reaction mixture was quenched with water (20 mL) and extracted with DCM (20 mL×3). The combined organic layers were washed with brine, dried over anhydrous Na₂SO₄, filtered and concentrated under reduced pressure. The residue was purified by silica gel chromatography (DCM/MeOH=50:1) to provide the desired product (2.6 g, 90% yield) as a colorless oil. MS (ESI) m/z=289.4.2 [M+H]⁺.

### Step 2. Synthesis of tert-butyl (7-oxoheptyl)carbamate

To a solution of tert-butyl (7-(methoxy(methyl)amino)-7-oxoheptyl)carbamate (2.1 g, 7.3 mmol) in MeOH (5 mL) was added lithium aluminium hydride (14.6 mL, 1 M in THF) dropwise at −78° C. The reaction mixture was warmed to 0° C. and stirred at this temperature for 30 min. The reaction was quenched with saturated NH₄Cl and extracted with EtOAc (20 mL×3). The combined organic layers were washed with brine, dried over Na₂SO₄, filtered and concentrated to provide the desired product (1.3 g, crude) as a light-yellow oil. MS (ESI) m/z=252.2 [M+Na]⁺.

### Step 3. Synthesis of 2-methyl-N-(6-methylpyridin-3-yl)-3-nitrobenzamide

To a solution of 2-methyl-3-nitrobenzoic acid (1.0 g, 5.5 mmol) in DMF (20 mL) were added 6-methylpyridin-3-amine (716 mg, 6.6 mmol), HATU (2.5 g, 6.6 mmol) and DIPEA (2.13 g, 16.5 mmol) at rt. After the reaction mixture was stirred at rt overnight, it was quenched with H₂O (50 mL) and extracted with EtOAc (50 mL×3). The combined organic layers were washed with brine, dried over Na₂SO₄, filtered and concentrated under reduced pressure. The residue was purified by silica gel column chromatography (petroleum ether/ethyl acetate=2:1) to provide the desired product (1.19 g, 80% yield) as a white solid. MS (ESI) m/z=272.1 [M+H]⁺.

### Step 4. Synthesis of 3-amino-2-methyl-N-(6-methylpyridin-3-yl)benzamide

To a solution of 2-methyl-N-(6-methylpyridin-3-yl)-3-nitrobenzamid (600 mg, 2.2 mmol) in THF (20 mL) was added Pd/C (10%, 120 mg). After stirring at rt for 8 h under H₂ atmosphere, the reaction mixture was filtered and the filtrate was concentrated under reduced pressure. The residue was purified by prep-HPLC to provide the desired product (560 mg, crude) as a white solid. MS (ESI) m/z=242.2 [M+H]⁺.

### Step 5. Synthesis of tert-butyl (7-((2-methyl-3-((6-methylpyridin-3-yl)carbamoyl)phenyl)amino)heptyl)carbamate

To a solution of 3-amino-2-methyl-N-(6-methylpyridin-3-yl)benzamide (350 mg, 1.4 mmol) in CHCl₃ (10 mL) were added tert-butyl (7-oxoheptyl)carbamate (321 mg, 1.4 mmol) and NaBH(OAc)₃ (890 mg, 4.2 mmol). After the reaction mixture was stirred at rt overnight, it was quenched with aq. NaHCO₃ and extracted with DCM (10 mL×3). The combined organic layers were washed with brine, dried over Na₂SO₄, filtered and concentrated under reduced pressure. The residue was purified by silica gel column chromatography (petroleum ether/ethyl acetate=1:1) to provide the desired product (98 mg, 15% yield) as a white solid. ¹HNMR (400 MHz, DMSO-d₆) δ 10.30 (s, 1H), 8.74 (d, J=2.4 Hz, 1H), 8.02 (dd, J=8.4, 2.4 Hz, 1H), 7.21 (d, J=8.4 Hz, 1H), 7.10 (t, J=7.6 Hz, 1H), 6.76-6.75 (m, 1H), 6.64-6.63 (m, 2H), 5.00 (t, J=4.8 Hz, 1H), 3.10 (q, J=6.8 Hz, 2H), 2.89 (q, J=6.8 Hz, 2H), 2.42 (s, 3H), 2.07 (s, 3H), 1.59-1.56 (m, 2H), 1.37-1.26 (m, 17H). MS (ESI) m/z=455.4 [M+H]⁺.

### Step 6. Synthesis of 3-((7-aminoheptyl)amino)-2-methyl-N-(6-methylpyridin-3-yl)benzamide

The title compound was synthesized following the standard procedure for preparing BL1-46 (TFA salt, 8.0 mg, 78% yield) as a yellow oil. MS (ESI) m/z 355.5 [M+H]⁺.

### Example 524. 3-((7-aminoheptyl)amino)-N-(1,5-dimethyl-1H-pyrazol-3-yl)-2-methylbenzamide (BL1-201)

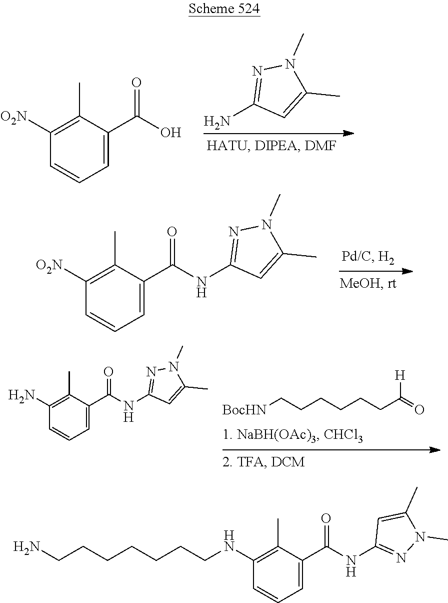

### Step 1. Synthesis of N-(1,5-dimethyl-1H-pyrazol-3-yl)-2-methyl-3-nitrobenzamide

The title compound was synthesized following the standard procedure for preparing BL1-200 (900 mg, 91% yield) as a white solid. MS (ESI) m/z 275.2 [M+H]⁺.

### Step 2. Synthesis of 3-amino-N-(1,5-dimethyl-1H-pyrazol-3-yl)-2-methylbenzamide

The title compound was synthesized following the standard procedure for preparing BL1-200 (370 mg, 99% yield) as a white solid. MS (ESI) m/z 245.2 [M+H]⁺.

### Step 3. Synthesis of tert-butyl (7-((3-((1,5-dimethyl-1H-pyrazol-3-yl)carbamoyl)-2-methylphenyl)amino)heptyl)carbamate

The title compound was synthesized following the standard procedure for preparing BL1-200 (109 mg, 24% yield) as a white solid. ¹HNMR (400 MHz, DMSO-d₆) δ 10.33 (s, 1H), 7.03 (t, J=8.0 Hz, 1H), 6.76 (t, J=5.2 Hz, 1H), 6.59-6.54 (m, 2H), 6.38 (s, 1H), 4.91-4.88 (m, 1H), 3.61 (s, 3H), 3.07 (q, J=6.8 Hz, 2H), 2.89 (q, J=6.8 Hz, 2H), 2.23 (s, 3H), 2.04 (s, 3H), 1.59-1.54 (m, 2H), 1.37-1.24 (m, 17H). MS (ESI) m/z=458.4 [M+H]⁺.

### Step 4. Synthesis of 3-((7-aminoheptyl)amino)-N-(1,5-dimethyl-1H-pyrazol-3-yl)-2-methylbenzamide

The title compound was synthesized following the standard procedure for preparing BL1-46 (TFA salt, 9.5 mg, 92% yield) as a yellow oil. MS (ESI) m/z 358.5 [M+H]⁺.

### Example 525. 2-((5-aminopentyl)amino)-N-(5-methylpyridin-2-yl)benzamide (BL1-202)

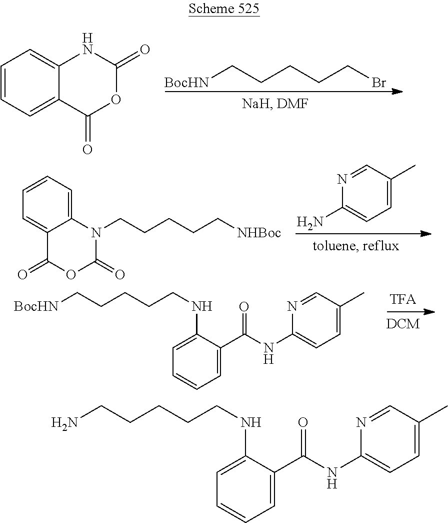

### Step 1. Synthesis of tert-butyl (5-(2,4-dioxo-2H-benzo[d][1,3]oxazin-1(4H)-yl)pentyl)carbamate

To a solution of 1H-benzo[d][1,3]oxazine-2,4-dione (326 mg, 2.0 mmol) in DMF (10 mL) was added NaH (60% in mineral oil, 96 mg, 2.4 mmol) at 0° C. The reaction mixture was warmed to rt and stirred for 2 h. A solution of tert-butyl (5-bromopentyl)carbamate (638 mg, 2.4 mmol) in DMF (2 mL) was added to the mixture dropwise. After stirring at rt overnight, the reaction was quenched with NH₄Cl solution and extracted with EtOAc (20 mL×3). The combined organic layers were washed with brine, dried over Na₂SO₄, filtered and concentrated under reduced pressure. The residue was purified by silica gel column chromatography (petroleum ether/ethyl acetate=2:1) to provide the desired product (180 mg, 26% yield) as a colorless oil. MS (ESI) m/z=349.2 [M+H]⁺.

### Step 2. Synthesis of tert-butyl (5-((2-((5-methylpyridin-2-yl)carbamoyl)phenyl)amino)pentyl)carbamate

A mixture of tert-butyl (5-((2-((5-methylpyridin-2-yl)carbamoyl)phenyl)amino)pentyl)carbamate (170 mg, 0.49 mmol) and 5-methylpyridin-2-amine (70 mg, 0.63 mmol) in toluene (10 mL) was heated to reflux overnight. After cooling down to rt, the mixture was concentrated under reduced pressure. The residue was purified by silica gel column chromatography (petroleum ether/ethyl acetate=5:1) to provide the desired product (100 mg, 50% yield) as a white solid. ¹HNMR (400 MHz, DMSO-d₆) δ 10.35 (s, 1H), 8.19 (d, J=2.0 Hz, 1H), 7.95 (t, J=8.4 Hz, 1H), 7.81-7.77 (m, 1H), 7.64-7.59 (m, 2H), 7.34-7.29 (m, 1H), 6.78-6.70 (m, 2H), 6.57 (t, J=7.2 Hz, 1H), 3.13-3.08 (m, 2H), 2.94-2.89 (m, 2H), 2.28 (s, 3H), 1.62-1.54 (m, 2H), 1.45-1.37 (m, 4H), 1.36 (s, 9H). MS (ESI) m/z=413.3 [M+H]⁺.

### Step 3. Synthesis of 2-((5-aminopentyl)amino)-N-(5-methylpyridin-2-yl)benzamide

The title compound was synthesized following the standard procedure for preparing BL1-46 (TFA salt, 10 mg, 96% yield) as a yellow oil. MS (ESI) m/z 313.4 [M+H]⁺.

### Example 526. 3-((7-aminoheptyl)amino)-2-methyl-N-(6-methylpyridazin-3-yl)benzamide (BL1-203)

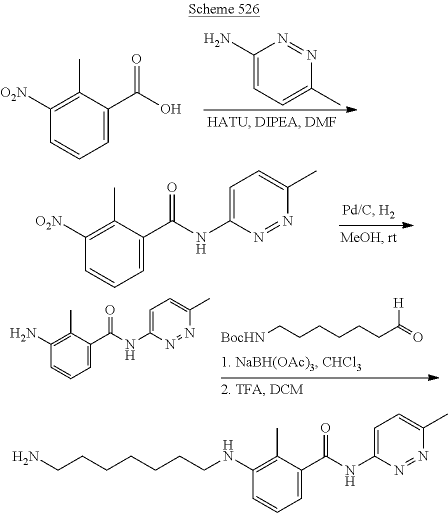

### Step 1. Synthesis of 2-methyl-N-(6-methylpyridazin-3-yl)-3-nitrobenzamide

The title compound was synthesized following the standard procedure for preparing BL1-200 (1.8 g, 71% yield) as a brown solid. MS (ESI) m/z 273.1 [M+H]⁺.

### Step 2. Synthesis of 3-amino-2-methyl-N-(6-methylpyridazin-3-yl)benzamide

The title compound was synthesized following the standard procedure for preparing BL1-200 (1.2 g, 63% yield) as a pale-yellow solid. MS (ESI) m/z 243.2 [M+H]⁺.

### Step 3. Synthesis of tert-butyl (7-((2-methyl-3-((6-methylpyridazin-3-yl)carbamoyl)phenyl)amino)heptyl)carbamate

The title compound was synthesized following the standard procedure for preparing BL1-200 (110 mg, 39% yield) as white solid. ¹HNMR (400 MHz, DMSO-d₆) δ 11.10 (s, 1H), 8.27 (d, J=8.8 Hz, 1H), 7.58 (d, J=9.2 Hz, 1H), 7.09 (t, J=8.0 Hz, 1H), 6.75 (brs, 1H), 6.69-6.62 (m, 2H), 4.97 (t, J=5.2 Hz, 1H), 3.12-3.07 (m, 2H), 2.92-2.88 (m, 2H), 2.58 (s, 3H), 2.01 (s, 3H), 1.61-1.56 (m, 2H), 1.37-1.21 (m, 17H). MS (ESI) m/z=456.4 [M+H]⁺.

### Step 4. Synthesis of 3-((7-aminoheptyl)amino)-2-methyl-N-(6-methylpyridazin-3-yl)benzamide

The title compound was synthesized following the standard procedure for preparing BL1-46 (TFA salt, 8.0 mg, 77% yield) as a yellow oil. MS (ESI) m/z 356.5 [M+H]⁺.

### Example 527. 3-((7-aminoheptyl)amino)-2-methyl-N-(5-methylpyridin-2-yl)benzamide (BL1-204)

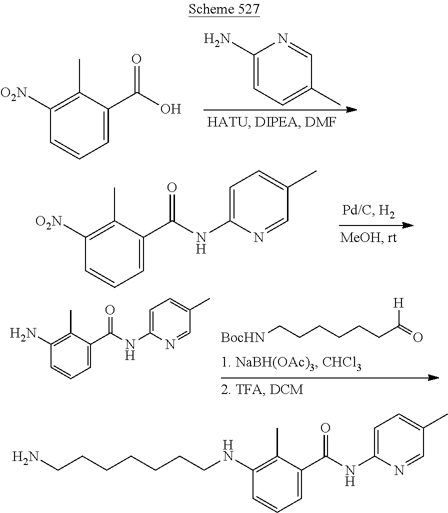

### Step 1. Synthesis of 2-methyl-N-(5-methylpyridin-2-yl)-3-nitrobenzamide

The title compound was synthesized following the standard procedure for preparing BL1-200 (2.1 g, 78% yield) as a pale-yellow solid. MS (ESI) m/z 272.1 [M+H]⁺.

### Step 2. Synthesis of 3-amino-2-methyl-N-(5-methylpyridin-2-yl)benzamide

The title compound was synthesized following the standard procedure for preparing BL1-200 (900 mg, 75% yield) as a white solid. MS (ESI) m/z 242.2 [M+H]⁺.

### Step 3. Synthesis of tert-butyl (7-((2-methyl-3-((5-methylpyridin-2-yl)carbamoyl)phenyl)amino)heptyl)carbamate

The title compound was synthesized following the standard procedure for preparing BL1-200 (160 mg, 35% yield) as a yellow solid. ¹HNMR (400 MHz, DMSO-d₆) δ 10.44 (s, 1H), 8.12 (s, 1H), 8.06 (d, J=7.6 Hz, 1H), 7.29 (d, J=7.6 Hz, 1H), 7.06 (t, J=8.0 Hz, 1H), 6.75 (s, 1H), 6.63-6.60 (m, 2H), 4.93 (t, J=4.8 Hz, 1H), 3.11-3.07 (m, 2H), 2.92-2.84 (m, 2H), 2.26 (s, 3H), 2.07 (s, 3H), 1.60-1.57 (m, 2H), 1.30-1.21 (m, 17H). MS (ESI) m/z=455.4 [M+H]⁺.

### Step 4. Synthesis of 3-((7-aminoheptyl)amino)-2-methyl-N-(5-methylpyridin-2-yl)benzamide

The title compound was synthesized following the standard procedure for preparing BL1-46 (TFA salt, 10.0 mg, 90% yield) as a yellow oil. MS (ESI) m/z 355.5 [M+H]⁺.

### Example 528. 3-((7-aminoheptyl)amino)-N-(6-methoxypyridazin-3-yl)-2-methylbenzamide (BL1-205)

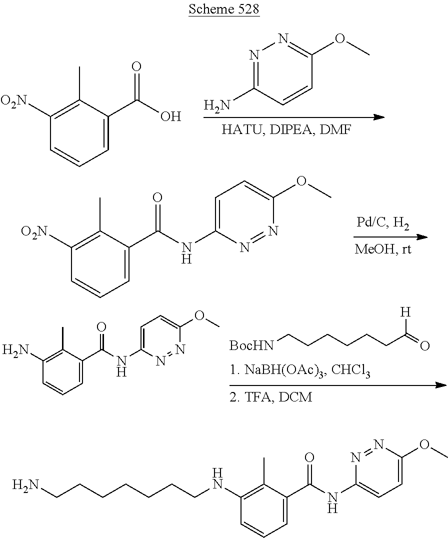

### Step 1. Synthesis of N-(6-methoxypyridazin-3-yl)-2-methyl-3-nitrobenzamide

The title compound was synthesized following the standard procedure for preparing BL1-200 (1.33 g, 70% yield) as a white solid. MS (ESI) m/z 289.1 [M+H]⁺.

### Step 2. Synthesis of 3-amino-N-(6-methoxypyridazin-3-yl)-2-methylbenzamide

The title compound was synthesized following the standard procedure for preparing BL1-200 (1.08 g, crude) as a white solid. MS (ESI) m/z 259.1 [M+H]⁺.

### Step 3. Synthesis of tert-butyl (7-((2-methyl-3-((5-methylpyridin-2-yl)carbamoyl)phenyl)amino)heptyl)carbamate

The title compound was synthesized following the standard procedure for preparing BL1-200 (270 mg, 38% yield) as a white solid. ¹HNMR (400 MHz, DMSO-d₆) δ 11.02 (s, 1H), 8.27 (d, J=9.2 Hz, 1H), 7.28 (d, J=9.2 Hz, 1H), 7.09 (t, J=7.6 Hz, 1H), 6.75-6.62 (m, 3H), 4.97 (t, J=4.8 Hz, 1H), 4.00 (s, 3H), 3.10 (q, J=6.4 Hz, 2H), 2.90 (q, J=6.4 Hz, 2H), 2.09 (s, 3H), 1.60-1.57 (m, 2H), 1.37-1.27 (m, 17H). MS (ESI) m/z=472.4 [M+H]⁺.

### Step 4. Synthesis of 3-((7-aminoheptyl)amino)-N-(6-methoxypyridazin-3-yl)-2-methylbenzamide

The title compound was synthesized following the standard procedure for preparing BL1-46 (TFA salt, 10.0 mg, 89% yield) as a yellow oil. MS (ESI) m/z 372.5 [M+H]⁺.

### Example 529. 2-(3-(2-(2-aminoethoxy)ethoxy)propanamido)-N-(6-(methylamino)pyridazin-3-yl)benzamide (BL1-206)

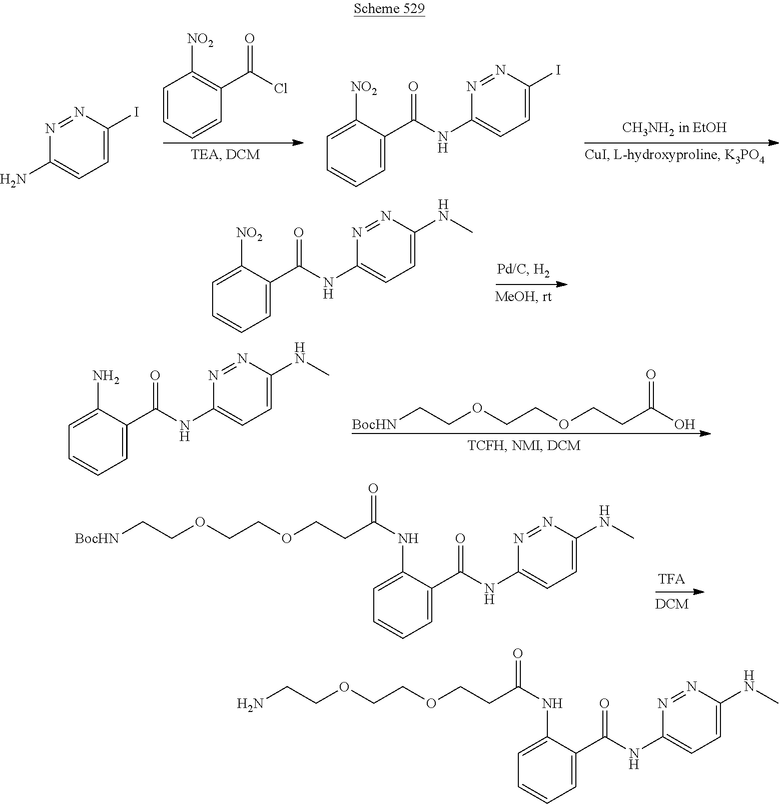

### Step 1. Synthesis of N-(6-iodopyridazin-3-yl)-2-nitrobenzamide

The title compound was synthesized following the standard procedure for preparing BL1-64 (1.0 g, 80% yield) as a yellow solid. MS (ESI) m/z 371.1 [M+H]⁺.

### Step 2. Synthesis of N-(6-(methylamino)pyridazin-3-yl)-2-nitrobenzamide

A mixture of N-(6-iodopyridazin-3-yl)-2-nitrobenzamide (500 mg, 1.35 mmol), CuI (30 mg, 0.14 mmol), L-hydroxyproline (40 mg, 0.28 mmol) and K₃PO₄ (850 mg, 4.0 mmol) in an ethanolic solution methylamine (10 ml of 30% solution) was stirred at 50° C. under N₂ atmosphere overnight. After cooling down to rt, the mixture was quenched with water (20 mL) and extracted with EtOAc (20 mL×3). The combined organic phase was dried over anhydrous sodium sulfate, filtered and concentrated under reduced pressure. The residue was purified by silica gel column chromatography (EA) and prep-HPLC to provide the desired product (150 mg, 40% yield) as a yellow solid. MS (ESI) m/z=274.2 [M+H]⁺.

The remaining steps were performed according to the procedures for preparing BL1-55 to provide the desired product (TFA salt, 15 mg, 45% yield) as a yellow oil. MS (ESI) m/z 403.4 [M+H]⁺.

### Example 530. 2-(3-(2-(2-aminoethoxy)ethoxy)propanamido)-N-(4,5-dimethylthiazol-2-yl)-6-methylnicotinamide (BL1-207)

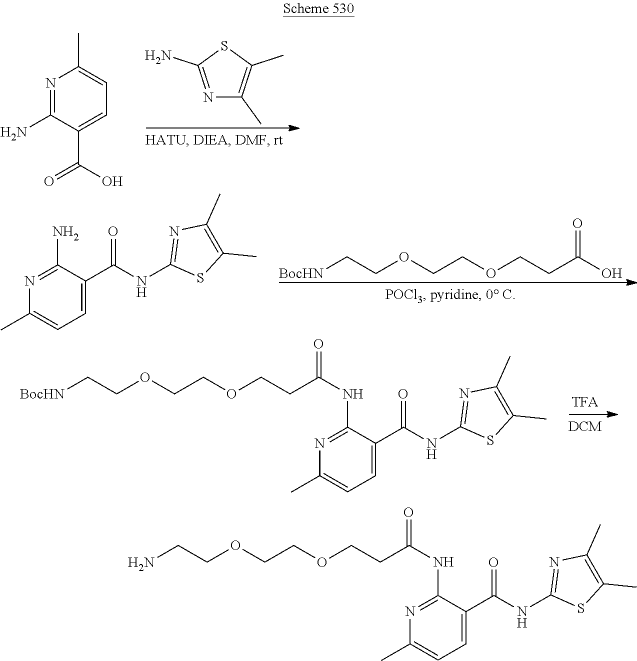

### Step 1. Synthesis of 2-amino-N-(4,5-dimethylthiazol-2-yl)-6-methylnicotinamide

A solution of 2-amino-6-methylnicotinic acid (500 mg, 3.29 mmol), 4,5-dimethylthiazol-2-amine (506 mg, 3.95 mmol), HATU (1.50 g, 3.95 mmol) and DIEA (849 mg, 6.58 mmol) in DMF (5 mL) was stirred at rt for 1 h. The mixture was diluted with water (50 mL) and extracted with EtOAc (50 mL×3). The combined organic phase was washed with brine, dried over Na₂SO₄, filtered and concentrated in vacuo. The residue was purified by silica gel chromatography (petroleum ether/EtOAc=1:1) to provide the title compound (400 mg, 46% yield) as a yellow solid. MS (ESI) m/z=263.2 [M+H]⁺.

### Step 2. Synthesis of tert-butyl (2-(2-(3-((3-((4,5-dimethylthiazol-2-yl)carbamoyl)-6-methylpyridin-2-yl)amino)-3-oxopropoxy)ethoxy)ethyl)carbamate

To a solution of 2-amino-N-(4,5-dimethylthiazol-2-yl)-6-methylnicotinamide (400 mg, 1.53 mmol), 2,2-dimethyl-4-oxo-3,8,11-trioxa-5-azatetradecan-14-oic acid (508 mg, 1.83 mmol) in pyridine (4 mL) was added POCl₃ (234 mg, 1.53 mmol) at 0° C. After the mixture was stirred at 0° C. for 1 h, it was quenched with MeOH (2 mL). The mixture was concentrated and purified by reverse-phase chromatography to provide the title compound (500 mg, 63% yield) as a yellow solid. MS (ESI) m/z=522.3 [M+H]⁺.

### Step 3. Synthesis of 2-(3-(2-(2-aminoethoxy)ethoxy)propanamido)-4-(methylamino)-N-(5-methylpyridin-2-yl)benzamide

A solution of tert-butyl (2-(2-(3-((3-((4,5-dimethylthiazol-2-yl)carbamoyl)-6-methylpyridin-2-yl)amino)-3-oxopropoxy)ethoxy)ethyl)carbamate (500 mg, 0.959 mmol) in TFA (4 mL) and DCM (4 mL) was stirred at rt for 2 h. The mixture was concentrated in vacuo. The residue was purified by prep-HPLC to provide the title compound (TFA salt, 400 mg, 78% yield) as a yellow oil. ¹HNMR (400 MHz, MeOD-d₄) δ 8.87-8.86 (m, 1H), 7.43 (d, J=8.0 Hz, 1H), 3.92 (t, J=5.6 Hz, 2H), 3.71-3.65 (m, 6H), 3.08 (t, J=5.2 Hz, 2H), 3.00 (t, J=6.0 Hz, 2H), 2.71 (s, 3H), 2.29 (s, 3H), 2.07 (s, 3H). MS (ESI) m/z=422.2 [M+H]⁺.

### Example 531. 2-(3-(2-(2-aminoethoxy)ethoxy)propanamido)-6-chloro-N-(5-methylpyridin-2-yl)benzamide (BL1-208)

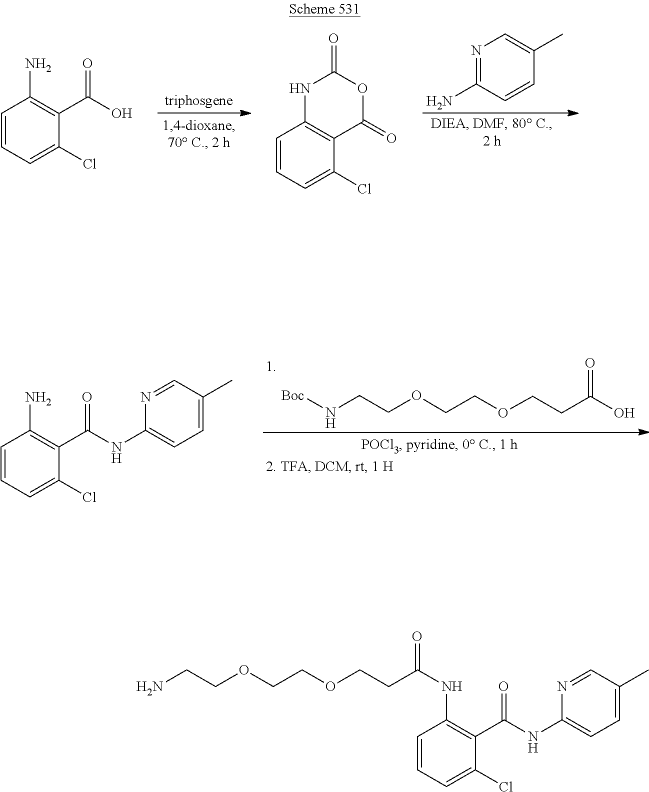

BL1-208 was synthesized following the standard procedures for preparing BL1-134 (TFA salt, 100 mg, 4% over 4 steps) as a yellow oil. ¹HNMR (400 MHz, DMSO-d₆) δ 10.89 (s, 1H), 9.43 (s, 1H), 8.18 (s, 1H), 8.09 (d, J=8.0 Hz, 1H), 7.71-7.67 (m, 5H), 7.39 (t, J=8.0 Hz, 1H), 7.33-7.30 (m, 1H), 3.47-3.46 (m, 6H), 2.96-2.92 (m, 2H), 2.55-2.52 (m, 2H), 2.28 (s, 3H), 2.01-1.99 (m, 2H). MS (ESI) m/z=421.3 [M+H]⁺.

### Example 532. 2-((7-aminoheptyl)amino)-N-(4,5-dimethylthiazol-2-yl)-6-methylnicotinamide (BL1-209)

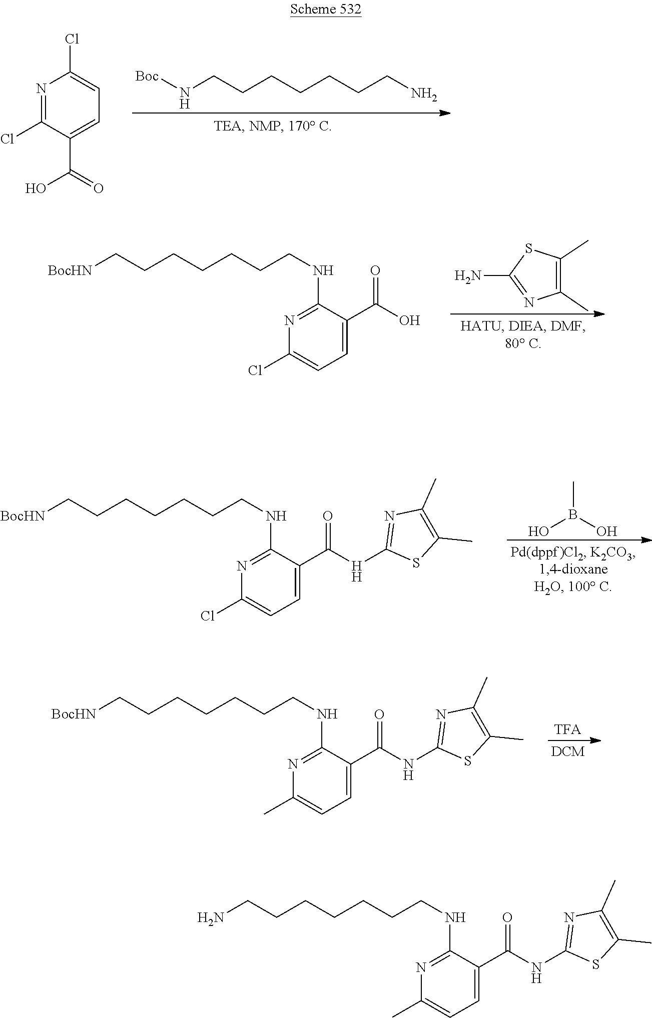

### Step 1. Synthesis of 2-((7-((tert-butoxycarbonyl)amino)heptyl)amino)-6-chloronicotinic acid

A solution of 2,6-dichloronicotinic acid (835 mg, 4.35 mmol), tert-butyl(7-aminoheptyl)carbamate (500 mg, 2.17 mmol) and TEA (658 mg, 6.52 mmol) in NMP (5 mL) was stirred at 170° C. for 2 h. After cooled to rt, the mixture was diluted with water (30 mL) and extracted with EtOAc (30 mL×3). The obtained organic phase was washed with brine, dried over Na₂SO₄, filtered and concentrated. The residue was purified by silica gel chromatography (petroleum ether/EtOAc=3:1) to provide the title compound (400 mg, 48% yield) as a white solid. MS (ESI) m/z=386.2 [M+H]⁺.

### Step 2. Synthesis of tert-butyl (7-((6-chloro-3-((4,5-dimethylthiazol-2-yl)carbamoyl)pyridin-2-yl)amino)heptyl)carbamate

A solution of 2-((7-((tert-butoxycarbonyl)amino)heptyl)amino)-6-chloronicotinic acid (400 mg, 1.04 mmol), 4,5-dimethylthiazol-2-amine (200 mg, 1.56 mmol), HATU (789 mg, 2.07 mmol) and DIEA (402 mg, 3.12 mmol) in DMF (4 mL) was stirred at 80° C. for 1 h. After cooled to rt, the mixture was diluted with water (30 mL) and extracted with EtOAc (30 mL×3). The obtained organic phase was washed with brine, dried over Na₂SO₄, filtered and concentrated. The residue was purified by silica gel chromatography (petroleum ether/EtOAc=3:1) to provide the title compound (200 mg, 39% yield) as a yellow solid. MS (ESI) m/z=496.2 [M+H]⁺.

### Step 3. Synthesis of tert-butyl (7-((3-((4,5-dimethylthiazol-2-yl)carbamoyl)-6-methylpyridin-2-yl)amino)heptyl)carbamate

A solution of tert-butyl (7-((6-chloro-3-((4,5-dimethylthiazol-2-yl)carbamoyl)pyridin-2-yl)amino)heptyl)carbamate (200 mg, 0.404 mmol), methylboronic acid (242 mg, 4.04 mmol), Pd(dppf)Cl₂ (30 mg, 0.0404 mmol) and K₂CO₃ (168 mg, 1.21 mmol) in 1,4-dioxane/H₂O (2 mL, 5:1) was stirred at 100° C. for 2 h under inert atmosphere. After cooled to rt, the mixture was diluted with water (30 mL) and extracted with EtOAc (30 mL×3). The obtained organic phase was washed with brine, dried over Na₂SO₄, filtered and concentrated. The residue was purified by silica gel chromatography (petroleum ether/EtOAc=3:1) to provide the title compound (140 mg, 73% yield) as a white solid. MS (ESI) m/z=476.4 [M+H]⁺.

### Step 4. Synthesis of 2-((7-aminoheptyl)amino)-N-(4,5-dimethylthiazol-2-yl)-6-methylnicotinamide

A solution of tert-butyl (7-((3-((4,5-dimethylthiazol-2-yl)carbamoyl)-6-methylpyridin-2-yl)amino)heptyl)carbamate (140 mg, 0.294 mmol) in DCM (1 mL) and TFA (1 mL) was stirred at rt for 2 h. The mixture was concentrated in high vacuum to provide the title compound (TFA salt, 100 mg, 70% yield) as a yellow solid. ¹HNMR (400 MHz, DMSO-d₆) δ 8.32-8.30 (m, 1H), 7.62 (brs, 3H), 6.57-7.64 (m, 1H), 3.48 (t, J=6.4 Hz, 2H), 2.80-2.75 (m, 2H), 2.39 (s, 3H), 2.24 (s, 3H), 2.19 (s, 3H), 1.63-1.61 (m, 2H), 1.54-1.50 (m, 2H), 1.35-1.33 (m, 6H). MS (ESI) m/z=376.3 [M+H]⁺.

### Example 533. 2-(3-(2-(2-aminoethoxy)ethoxy)propanamido)-4-(methylamino)-N-(5-methylpyridin-2-yl)benzamide (BL1-210)

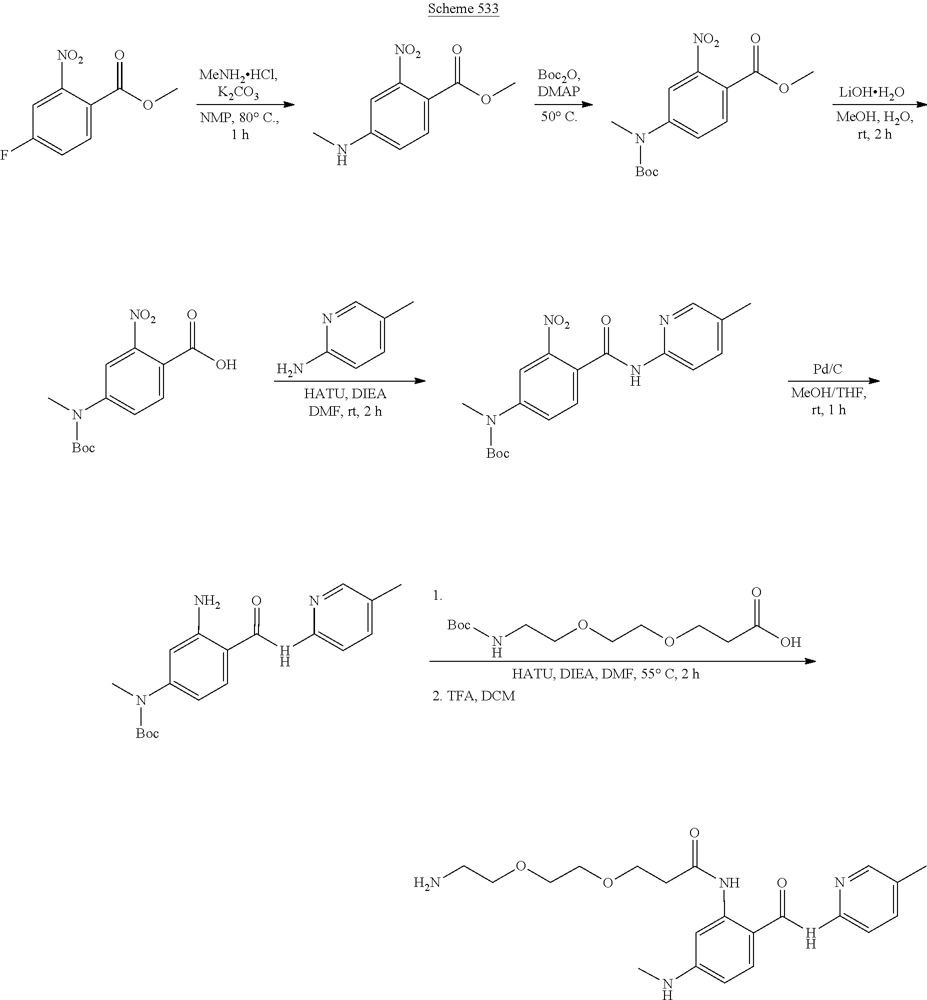

BL1-210 was synthesized following the standard procedure for preparing BL1-145 (TFA salt, 48.6 mg, 1% yield over 7 steps) as a yellow oil. ¹HNMR (400 MHz, DMSO-d₆) δ 11.53 (s, 1H), 10.49 (s, 1H), 8.23 (brs, 1H), 7.82-7.72 (m, 5H), 6.28 (dd, J=8.8, 2.0 Hz, 1H), 4.82-4.76 (m, 2H), 3.73 (t, J=6.0 Hz, 2H), 3.56-3.54 (m, 6H), 2.94-2.89 (m, 2H), 2.73 (s, 3H), 2.57 (t, J=6.0 Hz, 2H), 2.29 (s, 3H). MS (ESI) m/z=416.3 [M+H]⁺.

### Example 534. 2-((7-aminoheptyl)amino)-N,6-dimethylnicotinamide (BL1-211)

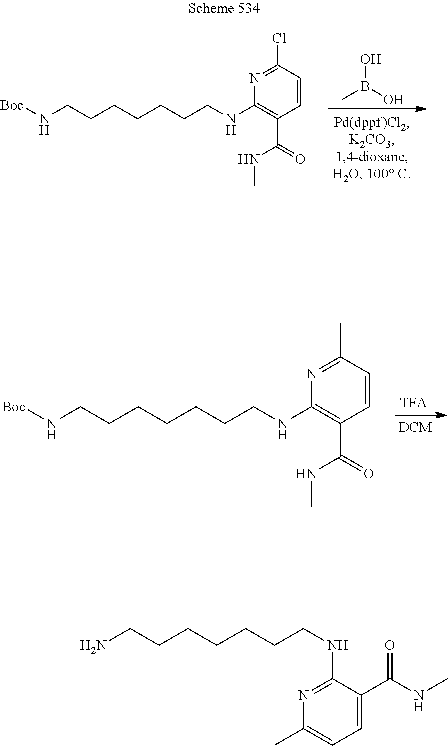

BL1-211 was synthesized following the standard procedure for preparing BL1-209 (TFA salt, 183 mg, 62% yield over 2 steps) as a yellow solid. ¹HNMR (400 MHz, DMSO-d₆) δ 8.94 (brs, 1H), 8.51 (brs, 1H), 7.93-7.92 (m, 1H), 7.66 (brs, 3H), 6.52 (d, J=7.6 Hz, 1H), 3.40 (t, J=7.2 Hz, 2H), 2.81-2.75 (m, 2H), 2.73 (d, J=4.8 Hz, 3H), 2.36 (s, 3H), 1.59-1.51 (m, 4H), 1.37-1.28 (m, 6H). MS (ESI) m/z=279.3 [M+H]⁺.

### Example 535. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)pioerazin-1-yl)acetamido)ethoxy)ethoxy)propanamido)-N-(4,5-dimethylthiazol-2-yl)-6-methylnicotinamide (CPD-336)

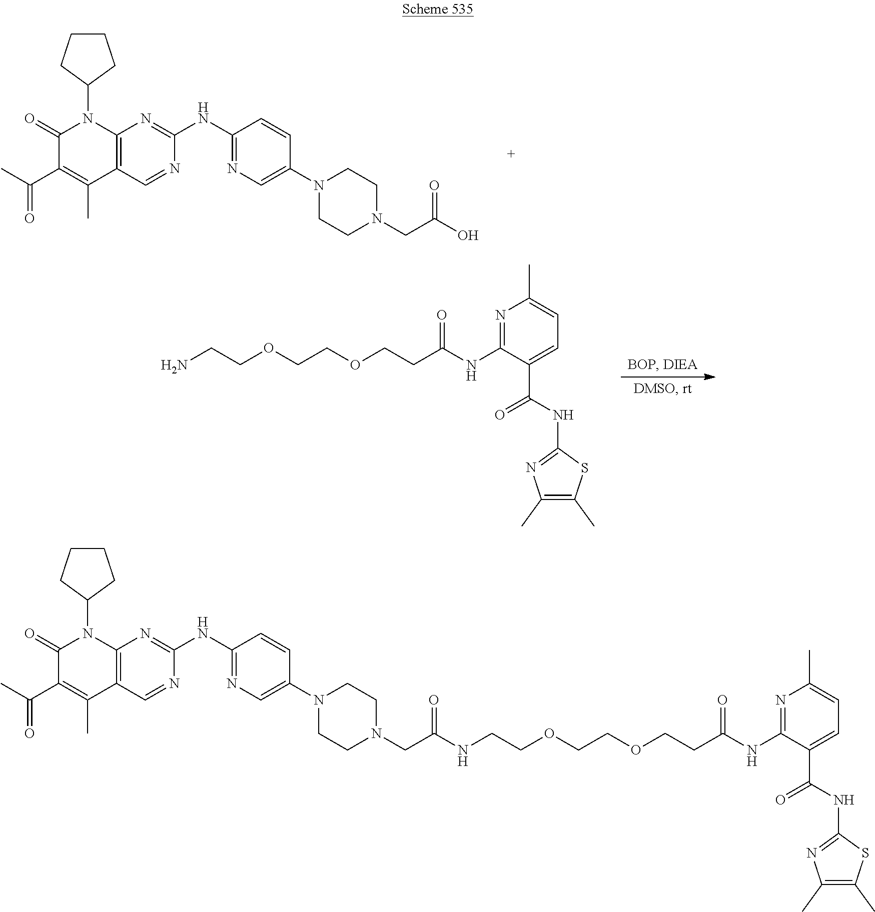 [SMILES: CC(=O)c1c(C)c2cnc(Nc3ccc(N4CCN(CC(=O)O)CC4)cn3)nc2n(C2CCCC2)c1=O ; Cc1ccc(C(=O)Nc2nc(C)c(C)s2)c(NC(=O)CCOCCOCCN)n1 ; CC(=O)c1c(C)c2cnc(Nc3ccc(N4CCN(CC(=O)NCCOCCOCCC(=O)Nc5nc(C)ccc5C(=O)Nc5nc(C)c(C)s5)CC4)cn3)nc2n(C2CCCC2)c1=O]

To a solution of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid (13.19 mg, 0.026 mmol) and 2-(3-(2-(2-aminoethoxy)ethoxy)propanamido)-N-(4,5-dimethylthiazol-2-yl)-6-methylnicotinamide (10 mg, 0.024 mmol) in DMSO (1.0 mL) were added BOP (19.37 mg, 0.095 mmol) and DIPEA (30.66 mg, 0.237 mmol, 39.21 uL). The reaction mixture was stirred at rt for 1 h. Upon completion, the reaction mixture was purified by prep-HPLC and reverse-phase chromatography to provide the title compound (TFA salt, 1.50 mg, 4% yield) as a yellow solid. MS (ESI) m/z=909.7 [M+H]⁺.

### Example 536. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4,5-dimethylthiazol-2-yl)-6-methylnicotinamide (CPD-337)

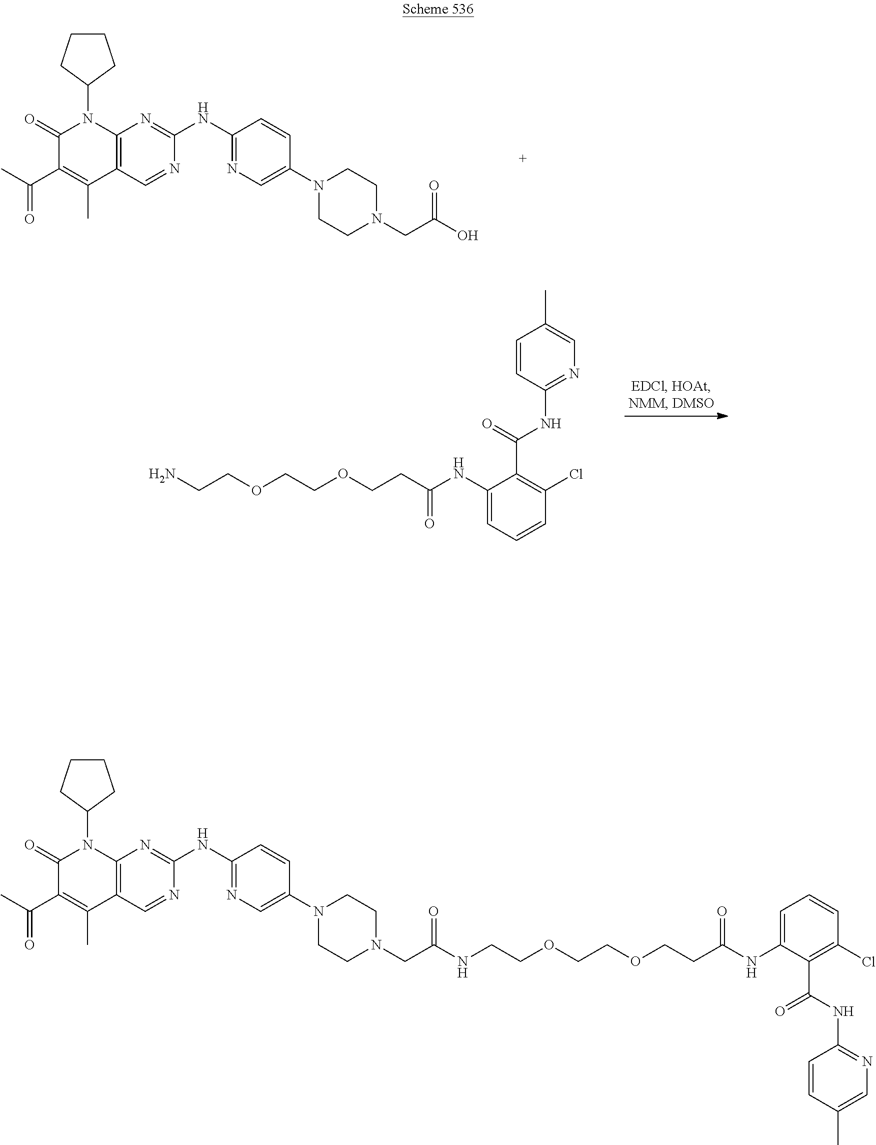 [SMILES: CC(=O)c1c(C)c2cnc(Nc3ccc(N4CCN(CC(=O)O)CC4)cn3)nc2n(C2CCCC2)c1=O ; Cc1ccc(NC(=O)c2c(Cl)cccc2NC(=O)CCOCCOCCN)nc1 ; CC(=O)c1c(C)c2cnc(Nc3ccc(N4CCN(CC(=O)NCCOCCOCCC(=O)Nc5cccc(Cl)c5C(=O)Nc5ccc(C)cn5)CC4)cn3)nc2n(C2CCCC2)c1=O]

CPD-337 was synthesized following the standard procedure for preparing CPD-042 (5.64 mg, 21% yield). MS (ESI) m/z=908.8 [M+H]⁺.

### Example 537. 2-((7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(4,5-dimethylthiazol-2-yl)-6-methylnicotinamide (CPD-338)

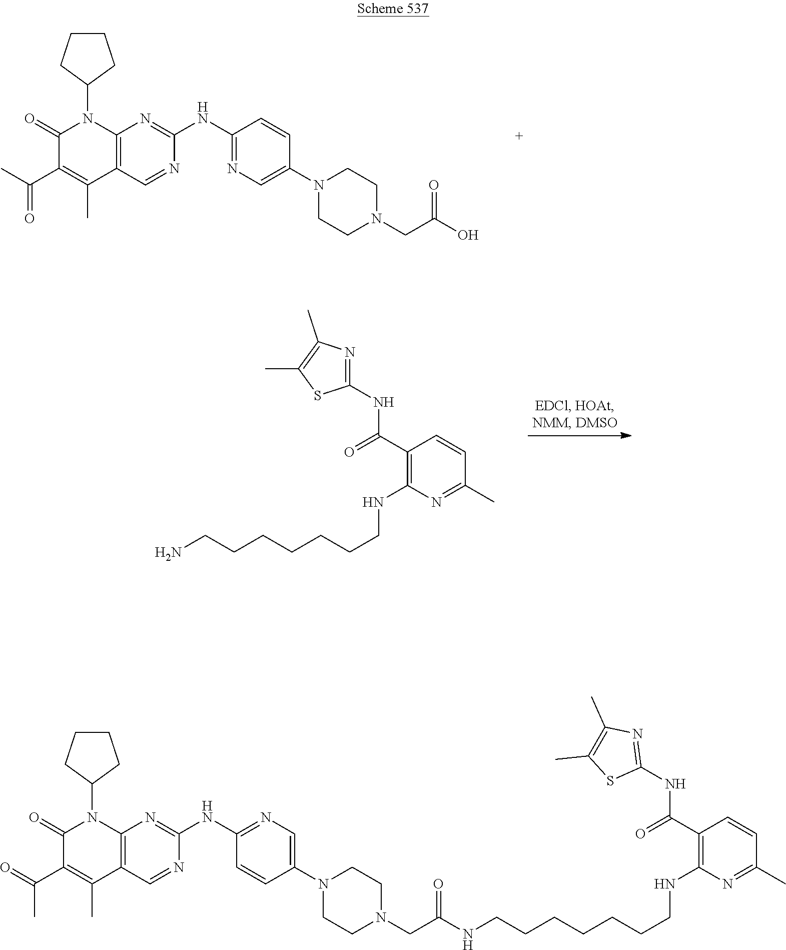 [SMILES: CC(=O)c1c(C)c2cnc(Nc3ccc(N4CCN(CC(=O)O)CC4)cn3)nc2n(C2CCCC2)c1=O ; Cc1ccc(C(=O)Nc2nc(C)c(C)s2)c(NCCCCCCCN)n1 ; CC(=O)c1c(C)c2cnc(Nc3ccc(N4CCN(CC(=O)NCCCCCCCNc5nc(C)ccc5C(=O)Nc5nc(C)c(C)s5)CC4)cn3)nc2n(C2CCCC2)c1=O]

CPD-338 was synthesized following the standard procedure for preparing CPD-042 (3.1 mg, 36% yield). MS (ESI) m/z=863.8 [M+H]⁺.

### Example 538. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-4-(methylamino)-N-(5-methylpyridin-2-yl)benzamide (CPD-339)

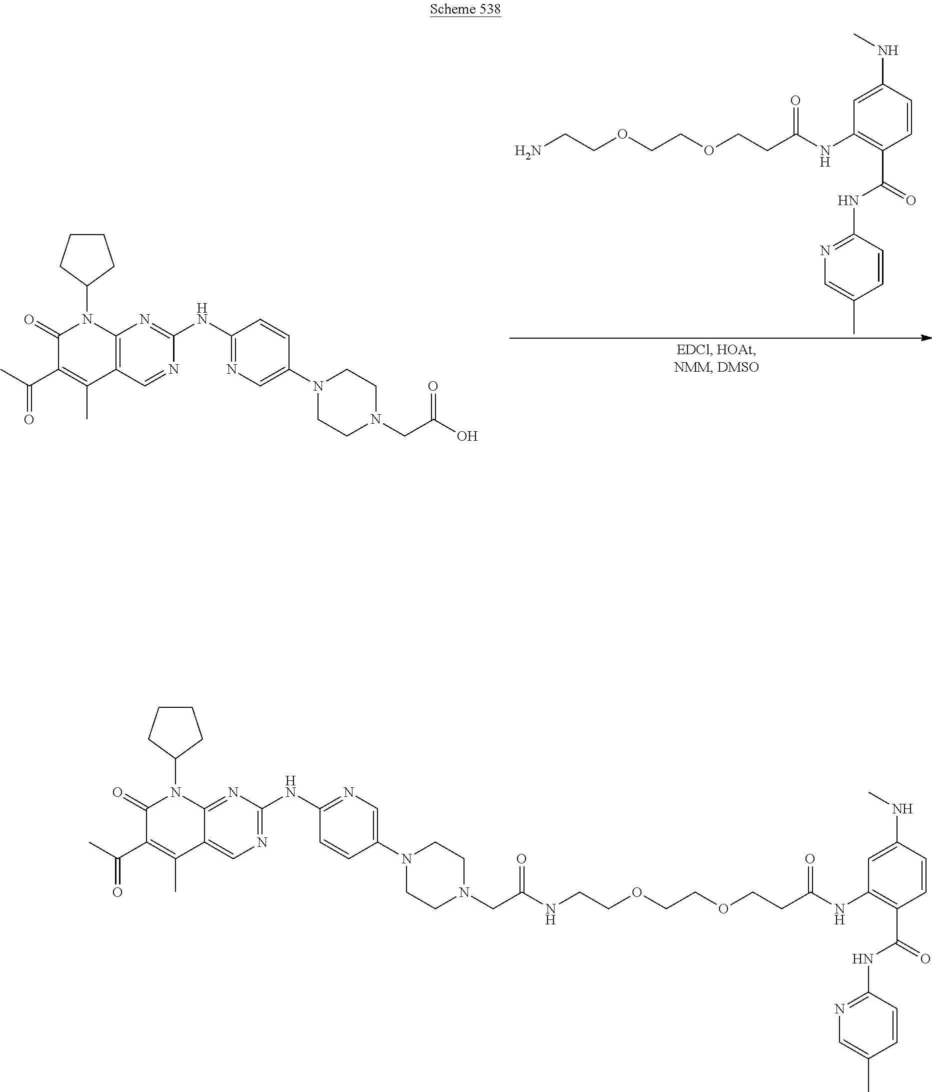 [SMILES: CC(=O)c1c(C)c2cnc(Nc3ccc(N4CCN(CC(=O)O)CC4)cn3)nc2n(C2CCCC2)c1=O ; CNc1ccc(C(=O)Nc2ccc(C)cn2)c(NC(=O)CCOCCOCCN)c1 ; CNc1ccc(C(=O)Nc2ccc(C)cn2)c(NC(=O)CCOCCOCCNC(=O)CN2CCN(c3ccc(Nc4ncc5c(C)c(C(C)=O)c(=O)n(C6CCCC6)c5n4)nc3)CC2)c1]

CPD-339 was synthesized following the standard procedure for preparing CPD-042 (8.93 mg, 26% yield). MS (ESI) m/z=903.8 [M+H]⁺.

### Example 539. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4,5-dimethylthiazol-2-yl)-6-methylnicotinamide (CPD-340)

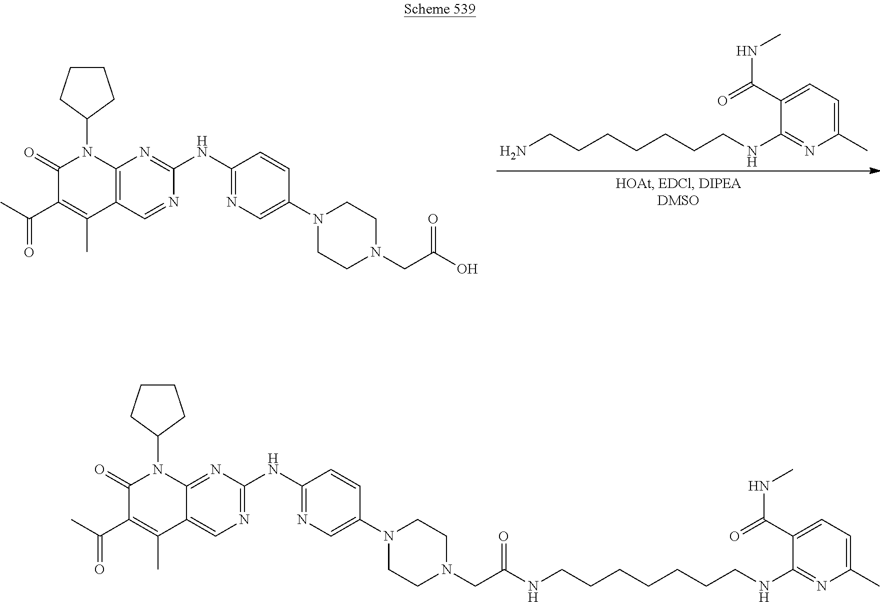 [SMILES: CC(=O)c1c(C)c2cnc(Nc3ccc(N4CCN(CC(=O)O)CC4)cn3)nc2n(C2CCCC2)c1=O ; CNC(=O)c1ccc(C)nc1NCCCCCCCN ; CNC(=O)c1ccc(C)nc1NCCCCCCCNC(=O)CN1CCN(c2ccc(Nc3ncc4c(C)c(C(C)=O)c(=O)n(C5CCCC5)c4n3)nc2)CC1]

CPD-340 was synthesized following the standard procedure for preparing CPD-042 (2.15 mg, 28% yield). MS (ESI) m/z=766.8 [M+H]⁺.

### Example 540. 3-((2-(2-(2-Aminoethoxy)ethoxy)ethyl)amino)-N-(6-methoxypyridazin-3-yl)-2-methylbenzamide (BL1-212)

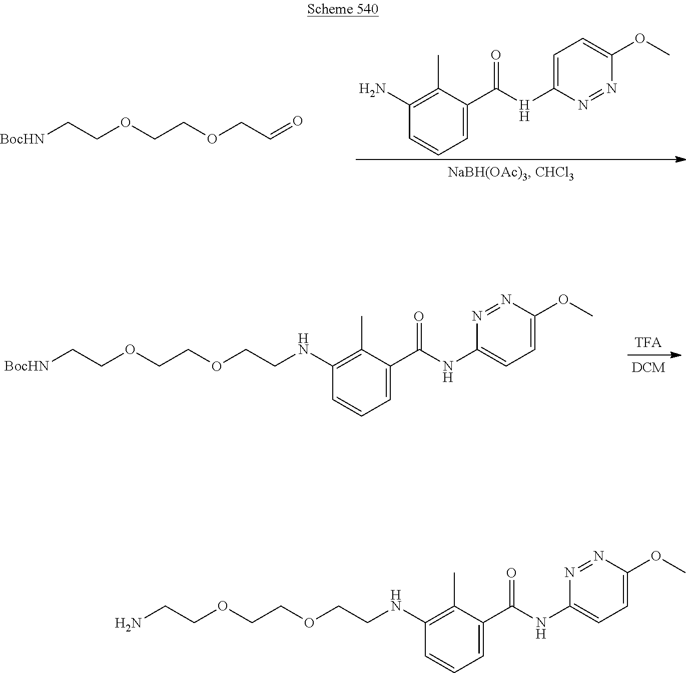

### Step 1. Synthesis of tert-butyl (2-(2-(2-((3-((6-methoxypyridazin-3-yl)carbamoyl)-2-methylphenyl)amino)ethoxy)ethoxy)ethyl)carbamate

To a solution of 3-amino-N-(6-methoxypyridazin-3-yl)-2-methylbenzamide (350 mg, 1.36 mmol) in CHCl₃ (10 mL) were added tert-butyl (2-(2-(2-oxoethoxy)ethoxy)ethyl)carbamate (402 mg, 1.63 mmol) and NaBH(OAc)₃ (577 mg, 2.72 mmol) at rt. After the reaction mixture was stirred at rt overnight, it was quenched with NaHCO₃ and extracted with EtOAc. The combined organic layers were washed with brine, dried over Na₂SO₄, filtered and concentrated. The residue was purified by silica gel column chromatography (petroleum ether/ethyl acetate=1:1) to provide the desired product (85 mg, 13% yield) as a white solid. ¹HNMR (400 MHz, DMSO-d₆) δ 11.04 (s, 1H), 8.28 (d, J=9.6 Hz, 1H), 7.28 (d, J=9.6 Hz, 1H), 7.11 (t, J=7.6 Hz, 1H), 6.75-6.70 (m, 3H), 4.93 (t, J=5.2 Hz, 1H), 4.00 (s, 3H), 3.61 (t, J=6.0 Hz, 2H), 3.56-3.52 (m, 4H), 3.39 (t, J=6.4 Hz, 2H), 3.31-3.28 (m, 2H), 3.07 (q, J=6.4 Hz, 2H), 2.10 (s, 3H), 1.36 (s, 9H). MS (ESI) m/z=490.4 [M+H]⁺.

### Step 2. Synthesis of 3-((2-(2-(2-aminoethoxy)ethoxy)ethyl)amino)-N-(6-methoxypyridazin-3-yl)-2-methylbenzamide

To a solution of tert-butyl (2-(2-(2-((3-((6-methoxypyridazin-3-yl)carbamoyl)-2-methylphenyl)amino)ethoxy)ethoxy)ethyl)carbamate (15 mg, 0.031 mmol) in DCM (2 mL) was added TFA (1 mL) at 0° C. After the reaction mixture was stirred at rt for 30 min, the solvents were removed under vacuum to give the desired product (15.4 mg, 97% yield) as TFA salt. MS (ESI) m/z=390.5 [M+H]⁺.

### Example 541. 2-(3-(2-(2-Aminoethoxy)ethoxy)propanamido)-N-(5-methoxypyridin-2-yl)benzamide (BL1-213)

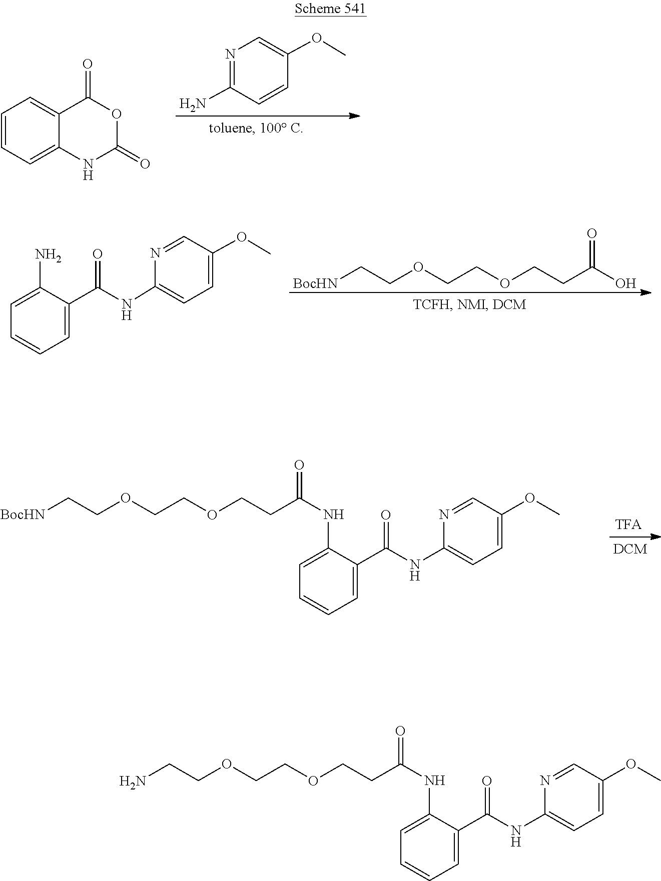

### Step 1. Synthesis of 2-amino-N-(5-methoxypyridin-2-yl)benzamide

A mixture of 5-methoxypyridin-2-amine (500 mg, 4.03 mmol) and 1H-benzo[d][1,3]oxazine-2,4-dione (789 mg, 4.84 mmol) in toluene (30 mL) was heated to reflux overnight. After cooling down to rt, the solvent was removed and the residue was purified by flash chromatography (petroleum ether/ethyl acetate=1:1) and prep-TLC to provide the desired product (138 mg, 14% yield) as a white solid. ¹HNMR (400 MHz, DMSO-d₆) δ 10.25 (s, 1H), 8.08 (d, J=2.8 Hz, 1H), 7.99 (d, J=8.8 Hz, 1H), 7.70 (dd, J=8.0, 1.6 Hz, 1H), 7.45 (dd, J=8.8, 3.2 Hz, 1H), 7.21-7.16 (m, 1H), 6.74 (dd, J=8.4, 0.8 Hz, 1H), 6.56-6.52 (m, 1H), 6.40 (s, 2H), 3.83 (s, 3H). MS (ESI) m/z=244.1 [M+H]⁺.

### Step 2. Synthesis of tert-butyl (2-(2-(3-((2-((5-methoxypyridin-2-yl)carbamoyl)phenyl)amino)-3-oxopropoxy)ethoxy)ethyl)carbamate

To a mixture of 2-amino-N-(5-methoxy-2-pyridyl)benzamide (20 mg, 0.082 mmol) and 2,2-dimethyl-4-oxo-3,8,11-trioxa-5-azatetradecan-14-oic acid (34.2 mg, 0.123 mmol) in DCM (10 mL) were added NMI (33.7 mg, 0.41 mmol) and TCFH (5.93 mg, 0.16 mmol) at 0° C. After the reaction mixture was stirred at rt for 3 h, it was concentrated and purified by silica gel flash chromatography to provide the desired product (25 mg, 61% yield) as a white solid. MS (ESI) m/z=503.6 [M+H]⁺.

### Step 3. Synthesis of 2-(3-(2-(2-aminoethoxy)ethoxy)propanamido)-N-(5-methoxypyridin-2-yl)benzamide

To a solution of tert-butyl (2-(2-(3-((2-((5-methoxypyridin-2-yl)carbamoyl)phenyl)amino)-3-oxopropoxy)ethoxy)ethyl)carbamate (25 mg, 0.05 mmol) in DCM (2 mL) was added TFA (1 mL) at 0° C. After the reaction mixture was stirred at rt for 30 min, the solvents were removed under vacuum to provide the desired product (25.7 mg, 70% yield) as TFA salt. MS (ESI) m/z=403.5 [M+H]⁺.

### Example 542. 2-(3-(2-(2-Aminoethoxy)ethoxy)propanamido)-N-(5-methoxypyridin-2-yl)benzamide (BL1-214)

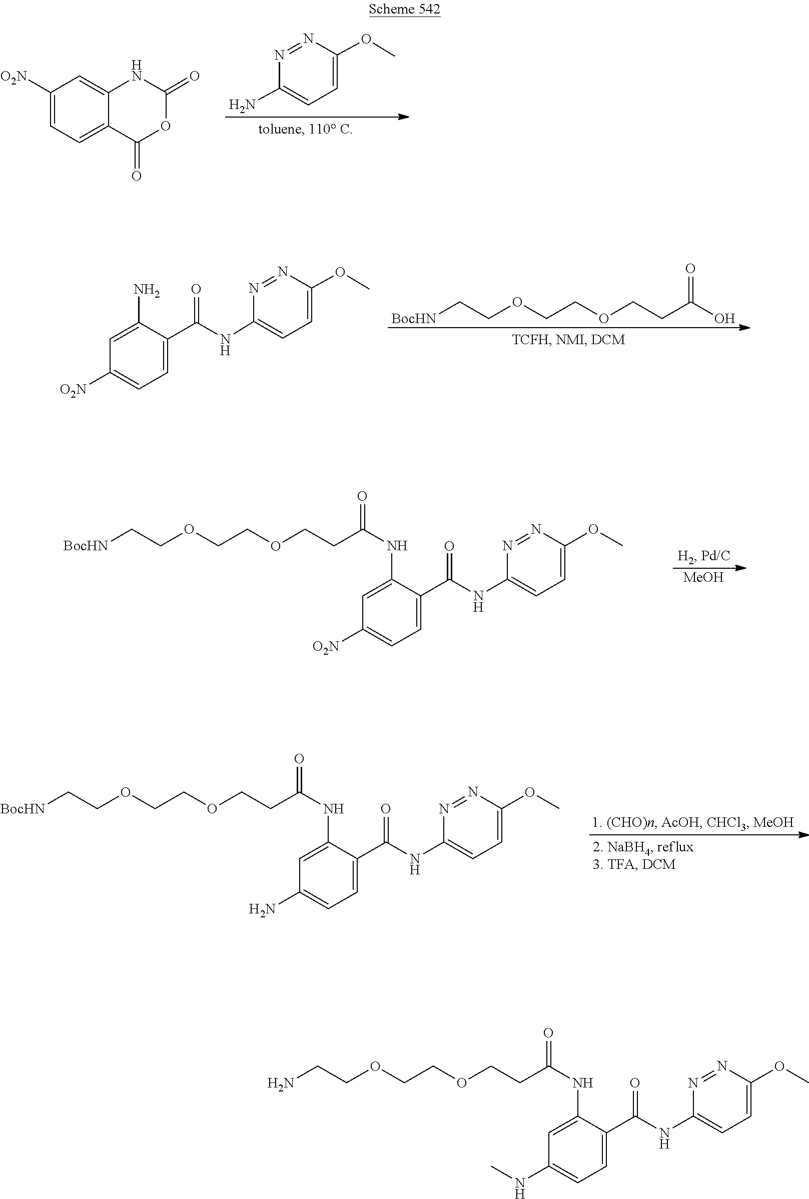

### Step 1. Synthesis of 2-amino-N-(6-methoxypyridazin-3-yl)-4-nitrobenzamide

A solution of 7-nitro-1H-benzo[d][1,3]oxazine-2,4-dione (4.2 g, 20 mmol) and 6-methoxypyridazin-3-amine (3.0 g, 24 mmol) in toluene (80 mL) was stirred at 110° C. overnight. The mixture was concentrated and the residue was purified by silica gel column chromatography (DCM/MeOH=100:1) to provide the desired product (4.8 g, 83% yield) as a white solid. MS (ESI) m/z=290.3 [M+H]⁺.

### Step 2. Synthesis of tert-butyl (2-(2-(3-((2-((6-methoxypyridazin-3-yl)carbamoyl)-5-nitrophenyl)amino)-3-oxopropoxy)ethoxy)ethyl)carbamate

To a solution of 2-amino-N-(6-methoxypyridazin-3-yl)-4-nitrobenzamide (1.25 g, 3.61 mmol) and 2,2-dimethyl-4-oxo-3,8,11-trioxa-5-azatetradecan-14-oic acid (1.0 g, 4.33 mmol) in CH₂Cl₂ (20 mL) were added TCFH (1.52 g, 5.4 mmol) and NMI (820 mg, 10.0 mmol). After the reaction mixture was stirred at rt overnight, it was quenched with NH₄Cl solution and extracted with DCM. The combined organic layers were washed with brine, dried over Na₂SO₄, filtered and concentrated. The residue was purified by silica gel column chromatography (petroleum ether/ethyl acetate=3:1) to provide the title compound (800 mg, 41% yield) as a yellow solid. MS (ESI) m/z=549.3 [M+H]⁺.

### Step 3. Synthesis of tert-butyl (2-(2-(3-((5-amino-2-((6-methoxypyridazin-3-yl)carbamoyl)phenyl)amino)-3-oxopropoxy)ethoxy)ethyl)carbamate

To a solution of tert-butyl (2-(2-(3-((2-((6-methoxypyridazin-3-yl)carbamoyl)-5-nitrophenyl)amino)-3-oxopropoxy)ethoxy)ethyl)carbamate (800 mg, 1.46 mmol) in MeOH (15 mL) was added Pd/C (80.0 mg). The reaction mixture was stirred at rt under H₂ atmosphere overnight. The mixture was filtered and the filtrate was concentrated to provide the crude product, which was purified by flash chromatography (0-100% EtOAc in petroleum ether) to provide the desired product (400 mg, 53% yield) as a yellow solid. MS (ESI) m/z=519.2 [M+H]⁺.

### Step 4. Synthesis of tert-butyl (2-(2-(3-((2-((6-methoxypyridazin-3-yl)carbamoyl)-5-(methylamino)phenyl)amino)-3-oxopropoxy)ethoxy)ethyl)carbamate

To a solution of tert-butyl (2-(2-(3-((5-amino-2-((6-methoxypyridazin-3-yl)carbamoyl)phenyl)amino)-3-oxopropoxy)ethoxy)ethyl)carbamate (330 mg, 0.64 mmol) in CHCl₃ (10.0 mL) and MeOH (10.0 mL) were added paraformaldehyde (30.0 mg) and one drop of AcOH. After the reaction mixture was stirred at rt overnight, NaBH₄ (190 mg, 5.0 mmol) was added. The mixture was heated to reflux overnight. Then it was quenched with NH₄Cl solution and extracted with DCM. The combined organic layers were washed with brine, dried over Na₂SO₄, filtered and concentrated. The residue was purified by silica gel column chromatography (0-100% EtOAc in petroleum ether) to provide the title compound (180 mg, 50% yield) as a yellow solid. ¹HNMR (400 MHz, DMSO-d₆) δ 11.51 (s, 1H), 10.82 (s, 1H), 8.08 (d, J=9.2 Hz, 1H), 7.85 (d, J=9.2 Hz, 1H), 7.78 (d, J=2.0 Hz, 1H), 7.25 (d, J=9.6 Hz, 1H), 6.70 (t, J=5.0 Hz, 1H), 6.63-6.58 (m, 1H), 6.29 (dd, J=8.8, 2.4 Hz, 1H), 4.01 (s, 3H), 3.69 (t, J=6.0 Hz, 2H), 3.51-3.44 (m, 4H), 3.33 (t, J=6.0 Hz, 2H), 3.04-2.99 (m, 2H), 2.73 (d, J=4.8 Hz, 3H), 2.54 (t, J=6.0 Hz, 2H), 1.35 (s, 9H). MS (ESI) m/z=533.4 [M+H]⁺.

### Step 5. Synthesis of 2-(3-(2-(2-aminoethoxy)ethoxy)propanamido)-N-(6-methoxypyridazin-3-yl)-4-(methylamino)benzamide

The title compound was synthesized following the procedure of step 2 for the preparation of BL1-212 to provide the desired product (18 mg, 88% yield) as TFA salt. MS (ESI) m/z=433.4 [M+H]⁺.

### Example 543. 2-(3-(4-(2-Aminoethyl)piperazin-1-yl)propanamido)-N-(6-methoxypyridazin-3-yl)benzamide (BL1-215)

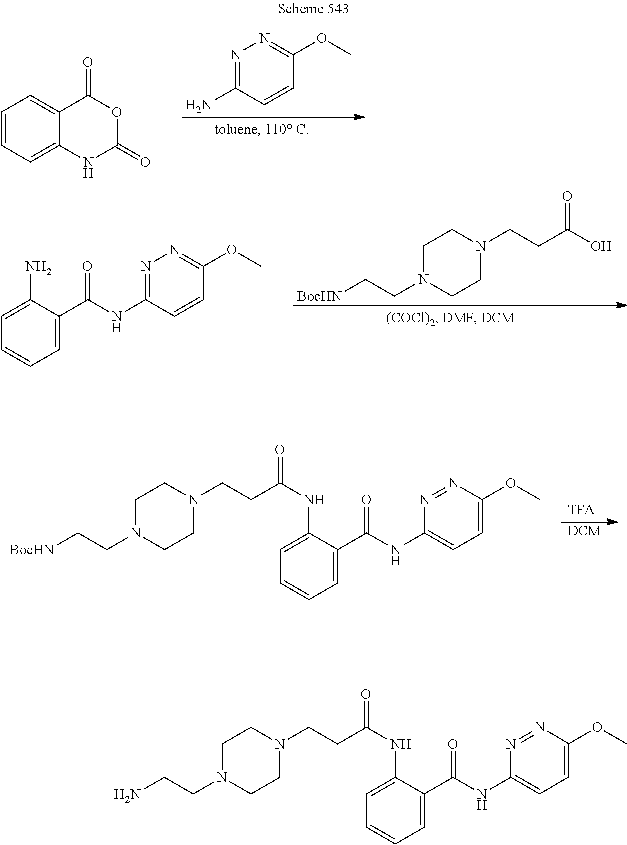

### Step 1. Synthesis of 2-amino-N-(6-methoxypyridazin-3-yl)benzamide

To a solution of 1H-benzo[d][1,3]oxazine-2,4-dione (4.9 g, 30 mmol) in toluene (40 mL) was added 6-methoxypyridazin-3-amine (4.1 g, 33 mmol). The reaction mixture was stirred at 110° C. under N₂ atmosphere overnight. After the reaction was cooled down to rt, the solvent was removed under reduced pressure and the residue was purified by flash chromatography (DCM/MeOH=100:1) to provide the desired product (5.8 g, 80% yield) as a yellow solid. MS (ESI) m/z=245.3 [M+H]⁺.

### Step 2. Synthesis of tert-butyl (2-(4-(3-((2-((6-methoxypyridazin-3-yl)carbamoyl)phenyl)amino)-3-oxopropyl)piperazin-1-yl)ethyl)carbamate

To a solution of 3-(4-(2-((tert-butoxycarbonyl)amino)ethyl)piperazin-1-yl)propanoic acid (460 mg, 1.5 mmol) in DCM (10 ml) was added oxalyl chloride (233 mg, 1.8 mmol), followed by 2 drops of DMF. The reaction mixture was stirred at rt for 30 min, before it was dropwise added into 2-amino-N-(6-methoxypyridazin-3-yl)benzamide (447 mg, 1.8 mmol) in DCM (5 mL). The resulting mixture was stirred at rt for 16 h. The solution was concentrated under reduced pressure and the residue was purified by flash chromatography (DCM/MeOH=50:1) to provide the desired product (280 mg, 35% yield) as a yellow solid. ¹HNMR (400 MHz, DMSO-d₆) δ 11.25 (brs, 1H), 10.48 (brs, 1H), 8.20 (d, J=9.2 Hz, 1H), 7.99 (d, J=8.0 Hz, 1H), 7.77 (dd, J=8.0, 1.2 Hz, 1H), 7.51 (d, J=6.8 Hz, 1H), 7.31 (d, J=9.2 Hz, 1H), 7.23-7.19 (m, 1H), 6.63 (brs, 1H), 4.01 (s, 3H), 3.00 (brs, 2H), 2.58-2.51 (m, 4H), 2.42-2.27 (m, 10H), 1.37 (s, 9H). MS (ESI) m/z=528.4 [M+H]⁺.

### Step 3. Synthesis of 2-(3-(4-(2-aminoethyl)piperazin-1-yl)propanamido)-N-(6-methoxypyridazin-3-yl)benzamide

The title compound was synthesized following the procedure of step 2 for the preparation of BL1-212 to provide the desired product (10 mg, 97% yield) as TFA salt. MS (ESI) m/z=428.4 [M+H]⁺.

### Example 544. 2-(5-(4-Aminopiperidin-1-yl)pentanamido)-N-(6-methoxypyridazin-3-yl)benzamide (BL1-216)

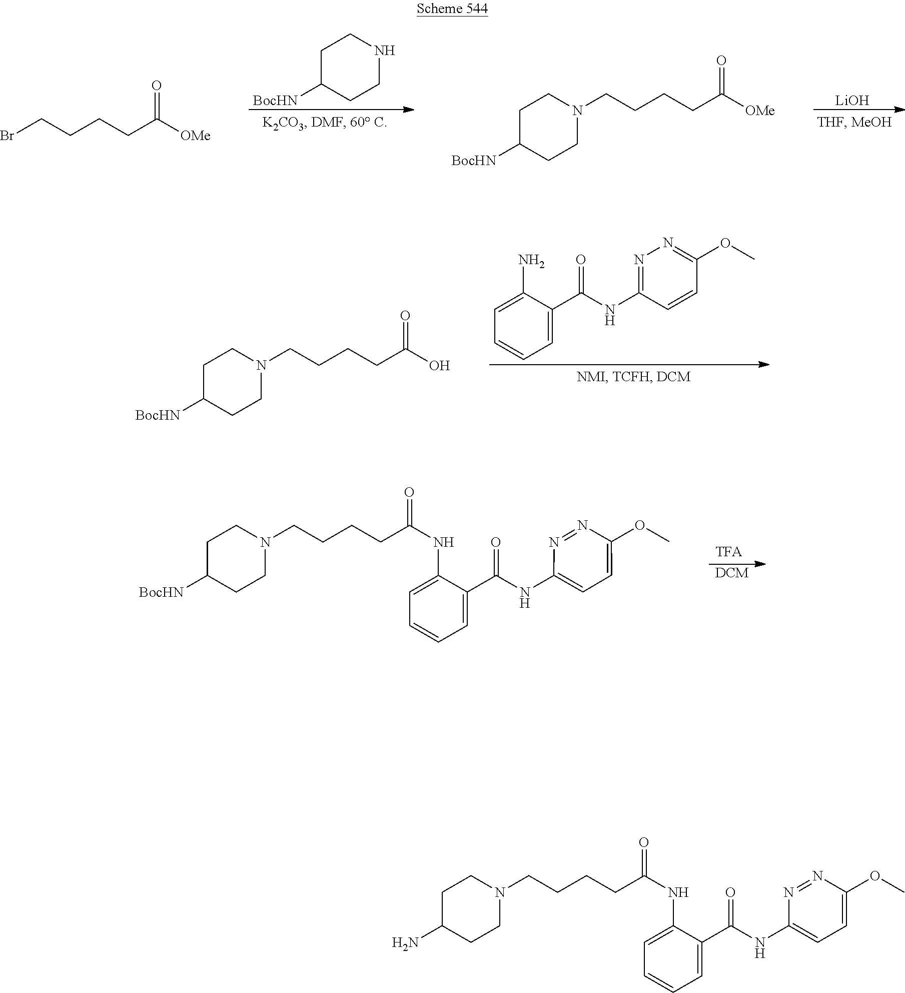

### Step 1. Synthesis of methyl 5-(4-((tert-butoxycarbonyl)amino)piperidin-1-yl)pentanoate

A mixture of methyl 5-bromopentanoate (1.0 g, 5.13 mmol), tert-butyl piperidin-4-ylcarbamate (1.23 g, 6.15 mmol) and K₂CO₃ (1.42 g, 10.26 mmol) in DMF (20 mL) was heated at 60° C. overnight. After cooling down to rt, the mixture was filtered. The filtrate was concentrated under reduced pressure. The residue was purified by silica gel column chromatography (DCM/MeOH=1:1) to provide the desired product (800 mg, 50% yield) as a yellow solid. MS (ESI) m/z=315.2 [M+H]⁺.

### Step 2. Synthesis of 5-(4-((tert-butoxycarbonyl)amino)piperidin-1-yl)pentanoic acid

To a solution of methyl 5-(4-((tert-butoxycarbonyl)amino)piperidin-1-yl)pentanoate (500 mg, 1.59 mmol) in THF (10.0 mL) and MeOH (3.0 mL) was added LiOH (2.0 M in H₂O, 1.5 mL, 3.0 mmol). After the reaction mixture was stirred at rt for 16 h, it was concentrated under reduced pressure. The residue was diluted with H₂O (5.0 mL) and acidified with 1M HCl to pH=5-6. The solution was lyophilized to provide the desired product (500 mg, crude) as a white solid, which was used directly in next step without further purification. MS (ESI) m/z=301.1 [M+H]⁺.

### Step 3. Synthesis of tert-butyl (1-(5-((2-((6-methoxypyridazin-3-yl)carbamoyl)phenyl)amino)-5-oxopentyl)piperidin-4-yl)carbamate

To a solution of 5-(4-((tert-butoxycarbonyl)amino)piperidin-1-yl)pentanoic acid (500 mg, crude) in CH₂Cl₂ (20 mL) were added 2-amino-N-(6-methoxypyridazin-3-yl)benzamide (250 mg, 1.03 mmol), TCFH (400 mg, 1.5 mmol) and NMI (250 mg, 3.0 mmol). After the reaction mixture was stirred at rt overnight, it was quenched with NH₄Cl solution and extracted with DCM. The combined organic layers were washed with brine, dried over Na₂SO₄, filtered and concentrated. The residue was purified by silica gel column chromatography (DCM/MeOH=10:1) and prep-HPLC to provide the desired product (120 mg, 23% yield) as a white solid. ¹HNMR (400 MHz, DMSO-d₆) δ 11.19 (s, 1H), 10.29 (s, 1H), 8.19 (d, J=9.6 Hz, 1H), 8.00 (d, J=8.0 Hz, 1H), 7.80 (dd, J=8.0, 1.6 Hz, 1H), 7.54-7.50 (m, 1H), 7.30 (d, J=9.2 Hz, 1H), 7.23-7.18 (m, 1H), 6.70 (d, J=7.6 Hz, 1H), 4.01 (s, 3H), 3.17-3.11 (m, 1H), 2.75-2.69 (m, 2H), 2.30 (t, J=7.4 Hz, 2H), 2.18 (t, J=7.2 Hz, 2H), 1.80 (t, J=10.8 Hz, 2H), 1.65-1.60 (m, 2H), 1.56-1.48 (m, 2H), 1.37 (s, 9H), 1.41-1.29 (m, 4H). MS (ESI) m/z=527.4 [M+H]⁺.

### Step 4. Synthesis of 2-(5-(4-aminopiperidin-1-yl)pentanamido)-N-(6-methoxypyridazin-3-yl)benzamide

The title compound was synthesized following the procedure of step 2 for the preparation of BL1-212 to provide the desired product (10 mg, 97% yield) as TFA salt. MS (ESI) m/z=427.4 [M+H]⁺.

### Example 545. 2-(2-(2-(4-Aminopiperidin-1-yl)ethoxy)acetamido)-N-(6-methoxypyridazin-3-yl)benzamide (BL1-217)

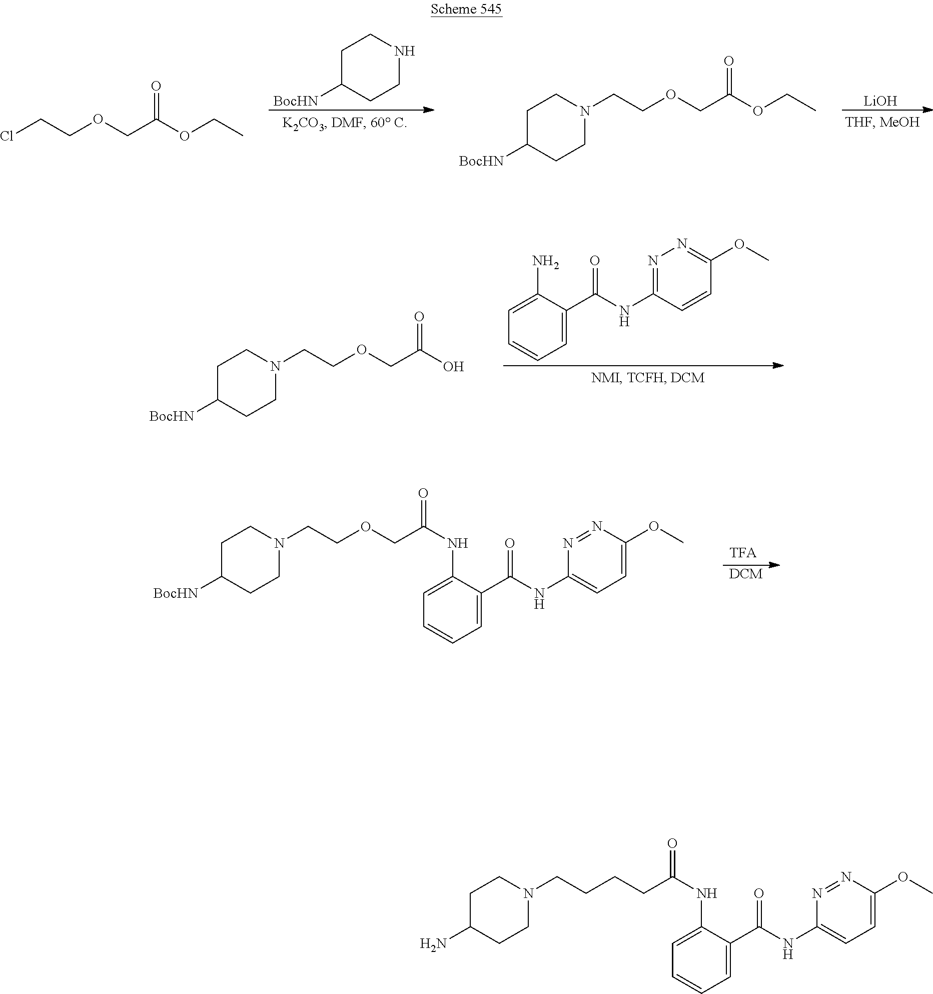

BL1-217 was synthesized following the procedures for preparing BL1-216 (13.3 mg, 4% yield over 4 steps) as TFA salt. MS (ESI) m/z=429.4 [M+H]⁺.

### Example 546. 2-(3-(2-(4-Aminopiperidin-1-yl)ethoxy)propanamido)-N-(6-methoxypyridazin-3-yl)benzamide (BL1-218)

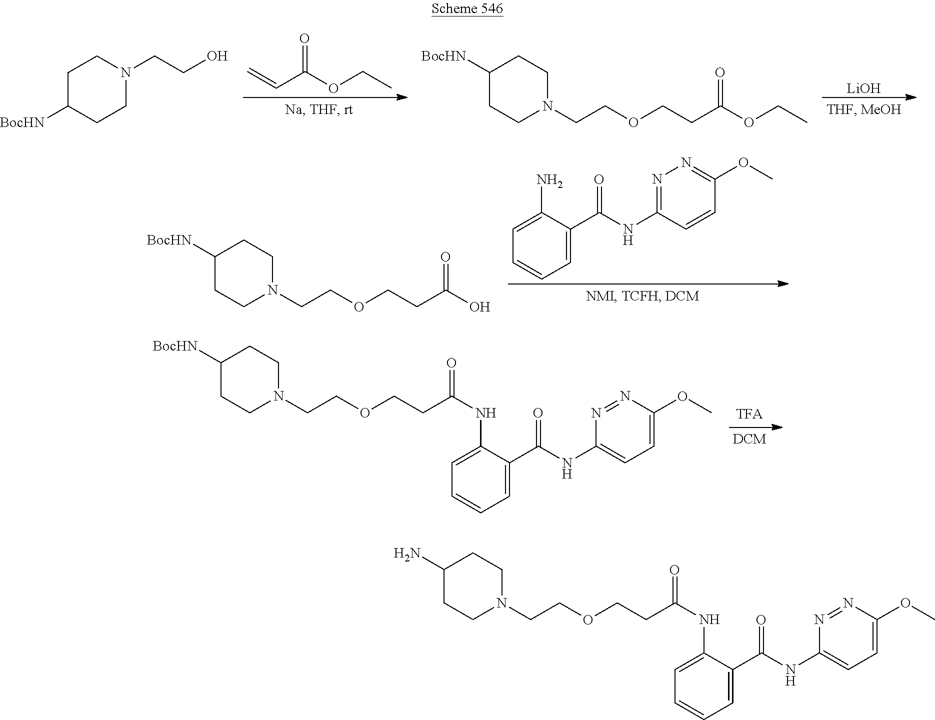

### Step 1. Synthesis of ethyl 3-(2-(4-((tert-butoxycarbonyl)amino)piperidin-1-yl)ethoxy)propanoate

To a solution of tert-butyl (1-(2-hydroxyethyl)piperidin-4-yl)carbamate (1.0 g, 4.1 mmol) and ethyl acrylate (620 mg, 6.13 mmol) in THF (20.0 mL) was added Na (100 mg, 4.1 mmol). After the reaction mixture was stirred at rt overnight, it was filtered and the filtrate was concentrated under reduced pressure. The residue was purified by flash chromatography (DCM/MeOH=10:1) to provide the desired product (500 mg, 27% yield) as a colorless oil.

MS (ESI) m/z=345.3 [M+H]⁺.

The remaining steps were performed according to the procedures for preparing BL1-216 to provide the desired product (10 mg, 22% yield over 3 steps) as TFA salt. MS (ESI) m/z=443.4 [M+H]⁺.

### Example 547. 2-(3-(2-(2-Aminoethoxy)ethoxy)propanamido)-N-(6-cyclopropoxypyridazin-3-yl)benzamide (BL1-220)

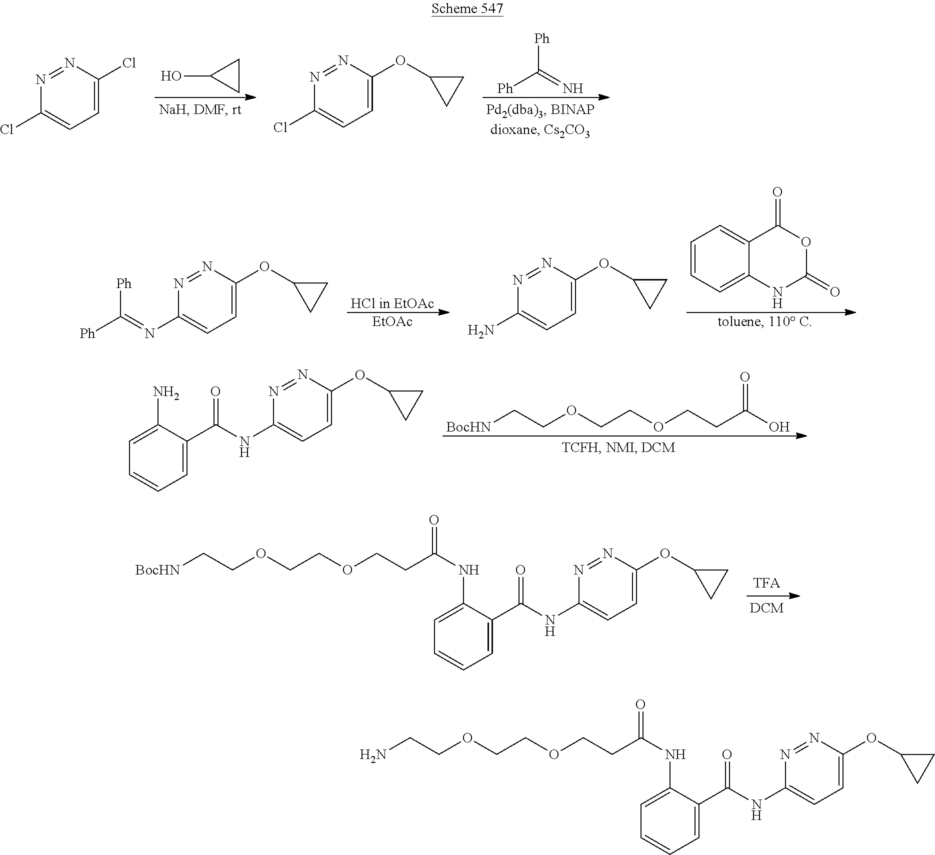

### Step 1. Synthesis of 3-chloro-6-cyclopropoxypyridazine

To a solution of cyclopropanol (580 mg, 10.0 mmol) in DMF (15.0 mL) was added NaH (60% in mineral oil, 200 mg, 5.0 mmol) at rt. After the mixture was stirred at rt for 30 min, 3,6-dichloropyridazine (750 mg, 5.0 mmol) in DMF (5.0 mL) was dropwise to the mixture. The resulting mixture was stirred at rt overnight before it was quenched with NH₄Cl solution and extracted with EtOAc. The combined organic layers were dried over Na₂SO₄, filtered and concentrated. The residue was purified by flash chromatography (petroleum/ethyl acetate=10:1) to provide the desired product (650 mg, 76% yield) as a white solid. MS (ESI) m/z=171.2 [M+H]⁺.

### Step 2. Synthesis of N-(6-cyclopropoxypyridazin-3-yl)-1,1-diphenylmethanimine

A mixture of 3-chloro-6-cyclopropoxypyridazine (340 mg, 2.0 mmol), diphenylmethanimine (380 mg, 2.08 mmol), Cs₂CO₃ (1.14 g, 3.48 mmol), BINAP (230 mg, 0.36 mmol) and Pd₂(dba)₃ (165 mg, 0.18 mmol) in 1,4-dioxane (20.0 mL) was heated to reflux overnight. After cooling down to rt, the reaction was quenched with NH₄Cl solution, and extracted with EtOAc. The combined organic layers were washed with brine, dried over Na₂SO₄, filtered and concentrated. The residue was purified by flash chromatography (petroleum/ethyl acetate=5:1) to provide the desired product (450 mg, 71% yield) as a yellow oil. MS (ESI) m/z=316.2 [M+H]⁺.

### Step 3. Synthesis of 6-cyclopropoxypyridazin-3-amine

To a solution of 6-cyclopropoxy-N-(diphenylmethylene)467yridazine-3-amine (450 mg, 1.43 mmol) in EtOAc (10.0 mL) was added HCl (6.0 M in EA, 10 mL). The reaction mixture was stirred at rt for 16 h, before it was concentrated under reduced pressure. The residue was diluted with H₂O (5.0 mL) and the pH value was adjusted to pH 5-6 with aq. NaOH (1M). The aqueous phase was extracted with EtOAc. The combined organic layers were washed with brine, dried over Na₂SO₄, filtered and concentrated. The residue was purified by flash chromatography to provide the desired product (150 mg, 69% yield) as a yellow oil. MS (ESI) m/z=152.1 [M+H]⁺.

The remaining steps were performed according to the procedures for preparing BL1-213 to provide the desired product (29.5 mg, 40% yield over 3 steps) as TFA salt. MS (ESI) m/z=430.4 [M+H]⁺.

### Example 548. 2-(3-(2-(2-Aminoethoxy)ethoxy)propanamido)-N-(6-isopropoxypyridazin-3-yl)benzamide (BL1-221)

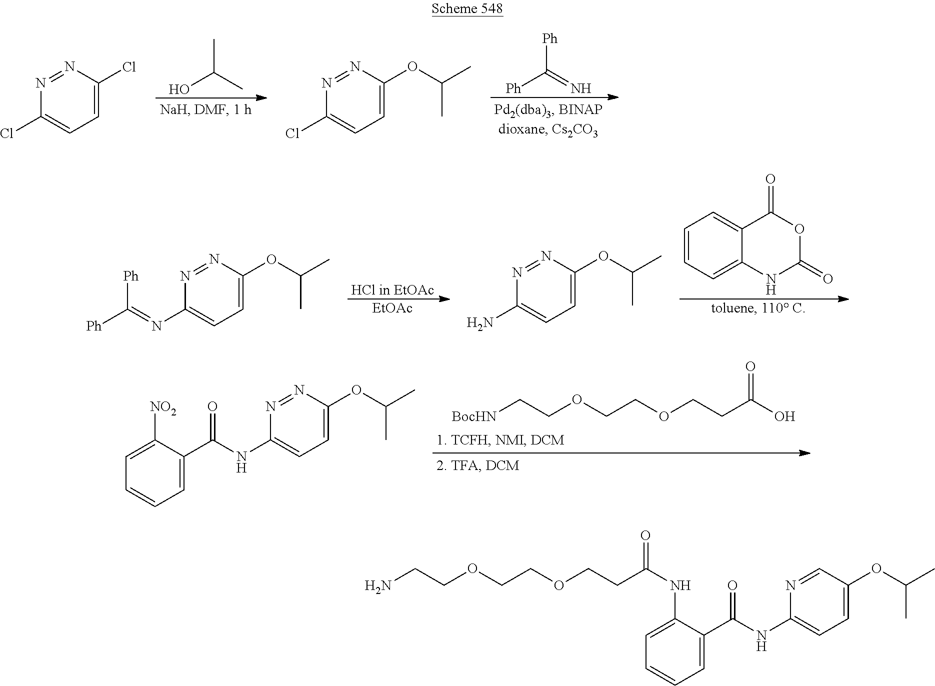

BL1-221 was synthesized following the procedures for preparing BL1-220 and BL1-213 (25 mg, 9% yield over 6 steps) as TFA salt. MS (ESI) m/z=432.4 [M+H]⁺.

### Example 549. 2-((5-Aminopentyl)amino)-N-(4,5-dimethylthiazol-2-yl)benzamide (BL1-223)

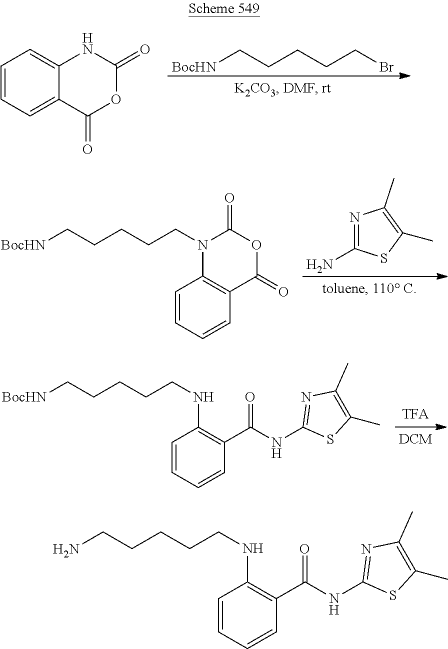

### Step 1. Synthesis of tert-butyl (5-(2,4-dioxo-2H-benzo[d][1,3]oxazin-1(4H)-yl)pentyl)carbamate

To a solution of 1H-benzo[d][1,3]oxazine-2,4-dione (300 mg, 1.84 mmol) in DMF (10 mL) were added potassium carbonate (508 mg, 3.68 mmol) and tert-butyl (5-bromopentyl)carbamate (488 mg, 1.84 mmol). The reaction mixture was stirred at rt for 8 h, before it was poured into water and extracted with EtOAc (30 mL×2). The combined organic layers were washed with brine, dried over anhydrous sodium sulfate, filtered and concentrated. The residue was purified by silica gel column chromatography (petroleum ether/ethyl acetate=1:1) to provide the desired product (301 mg, 47% yield) as a white solid. MS (ESI) m/z=349.2 [M+H]⁺.

### Step 2. Synthesis of tert-butyl (5-((2-((4,5-dimethylthiazol-2-yl)carbamoyl)phenyl)amino)pentyl)carbamate

To a solution of tert-butyl (5-(2,4-dioxo-2,4-dihydro-1H-benzo[d][1,3]oxazin-1-yl)pentyl)carbamate (300 mg, 0.86 mmol) in toluene (10 mL) was added 4,5-dimethylthiazol-2-amine (110 mg, 0.86 mmol) at rt. The reaction mixture was stirred at 110° C. overnight. After cooling down to rt, the mixture was diluted with water and extracted with EtOAc. The combined organic layers were washed with brine, dried over anhydrous sodium sulfate, filtered and concentrated. The residue was purified by silica gel column chromatography (petroleum ether/ethyl acetate=5:1) to afford the desired product (159 mg, 43% yield) as a white solid. ¹HNMR (400 MHz, DMSO-d₆) δ 12.03 (s, 1H), 7.89-7.83 (m, 2H), 7.35-7.31 (m, 1H), 6.79-6.72 (m, 2H), 6.58-6.54 (m, 1H), 3.29-3.12 (m, 2H), 2.95-2.90 (m, 2H), 2.25 (s, 3H), 2.19 (s, 3H), 1.63-1.560 (m, 2H), 1.44-1.31 (m, 13H). MS (ESI) m/z=433.3 [M+H]⁺.

### Step 3. Synthesis of 2-((5-aminopentyl)amino)-N-(4,5-dimethylthiazol-2-yl)benzamide

The title compound was synthesized following the procedure of step 2 for the preparation of BL1-212 to provide the desired product (11 mg, 97% yield) as TFA salt. MS (ESI) m/z=333.3 [M+H]⁺.

### Example 550. 2-((7-Aminoheptyl)amino)-N-(4,5-dimethylthiazol-2-yl)benzamide (BL1-224)

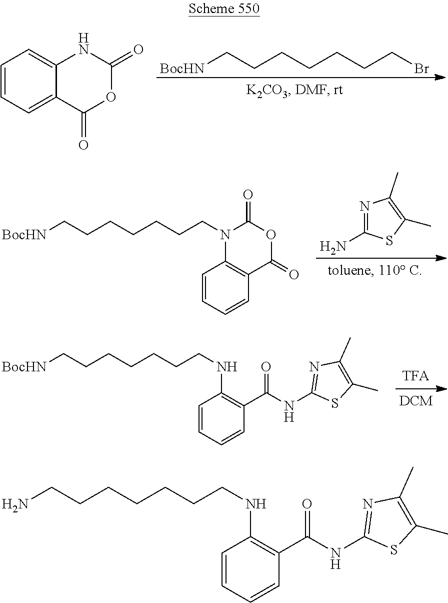

BL1-224 was synthesized following the procedures for preparing BL1-223 (15 mg, 32% yield over 3 steps) as TFA salt. MS (ESI) m/z=361.4 [M+H]⁺.

### Example 551. 2-(3-(2-(2-Aminoethoxy)ethoxy)propanamido)-4-(dimethylamino)-N-(6-methoxypyridazin-3-yl)benzamide (BL1-225)

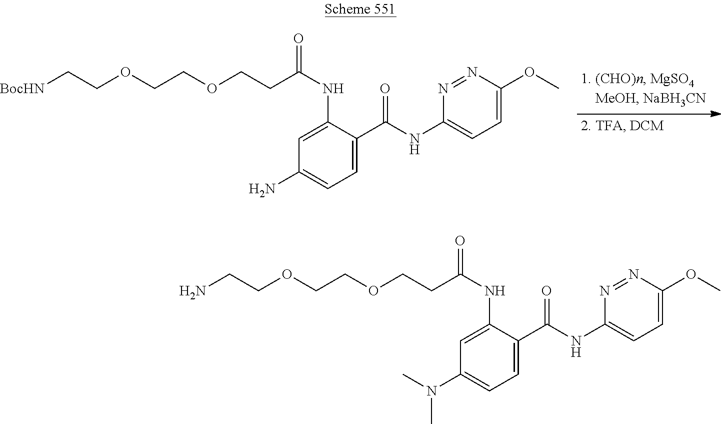 [SMILES: COc1ccc(NC(=O)c2ccc(N)cc2NC(=O)CCOCCOCCNC(=O)OC(C)(C)C)nn1 ; COc1ccc(NC(=O)c2ccc(N(C)C)cc2NC(=O)CCOCCOCCN)nn1]

### Step 1. Synthesis of tert-butyl (2-(2-(3-((5-(dimethylamino)-2-((6-methoxypyridazin-3-yl)carbamoyl)phenyl)amino)-3-oxopropoxy)ethoxy)ethyl)carbamate

To a solution of tert-butyl (2-(2-(3-((5-amino-2-((6-methoxypyridazin-3-yl)carbamoyl)phenyl)amino)-3-oxopropoxy)ethoxy)ethyl)carbamate (100 mg, 0.19 mmol) in MeOH (10.0 mL) were added MgSO₄ (120 mg, 1.0 mmol), paraformaldehyde (30.0 mg) and NaBH₃CN (38.0 mg, 1.0 mmol). The reaction mixture was stirred at 60° C. overnight. After cooling down to rt, the mixture was filtered and the filtrate was concentrated. The residue was purified by prep-HPLC to provide the desired product (45.0 mg, 43% yield) as a yellow solid. ¹HNMR (400 MHz, DMSO-d) b 11.48 (s, 1H), 10.92 (s, 1H), 8.09 (d, J=9.6 Hz, 1H), 7.95-7.84 (m, 2H), 7.27 (d, J=9.6 Hz, 1H), 6.72-6.67 (m, 1H), 6.47 (dd, J=8.8, 2.4 Hz, 1H), 4.01 (s, 3H), 3.69 (t, J=6.2 Hz, 2H), 3.51-3.43 (m, 4H), 3.33 (t, J=6.0 Hz, 2H), 3.03-3.01 (m, 2H), 3.00 (s, 6H), 2.55 (t, J=6.0 Hz, 2H), 1.35 (s, 9H). MS (ESI) m/z=547.4 [M+H]⁺.

### Step 2. Synthesis of 2-(3-(2-(2-aminoethoxy)ethoxy)propanamido)-4-(dimethylamino)-N-(6-methoxypyridazin-3-yl)benzamide

The title compound was synthesized following the procedure of step 2 for the preparation of BL1-212 to provide the desired product (11 mg, 19% yield) as TFA salt. MS (ESI) m/z=447.4 [M+H]⁺.

### Example 552. 2-(2-((trans-4-aminocyclohexyl)oxy)ethoxy)-N-(2-(6-methoxypyridazine-3-carbonyl)phenyl)acetamide (BL1-226)

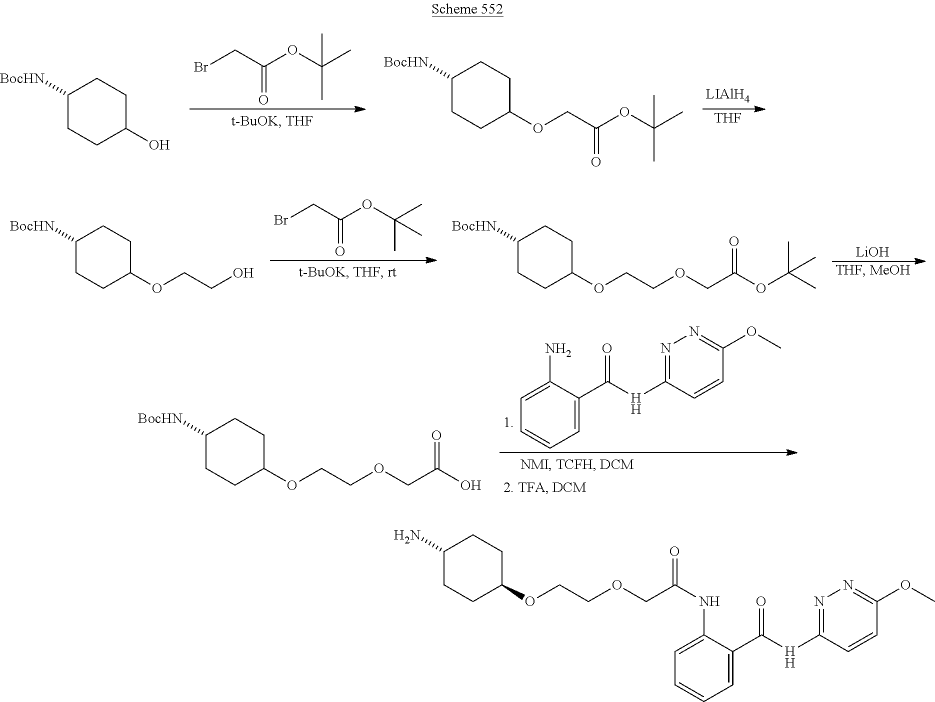

### Step 1. Synthesis of tert-butyl 2-((trans-4-((tert-butoxycarbonyl)amino)cyclohexyl)oxy)acetate

To a solution of tert-butyl (trans-4-hydroxycyclohexyl)carbamate (1.2 g, 5.57 mmol) in THF (20 mL) was added t-BuOK (750 mg, 6.69 mmol) at 0° C. After stirring at 0° C. for 20 min, a solution of tert-butyl 2-bromoacetate (1.3 g, 6.69 mmol) in THF (2.0 mL) was added to the mixture. The resulting mixture was stirred at rt overnight before it was quenched with NH₄Cl solution and extracted with DCM. The combined organic layers were dried over Na₂SO₄, filtered and concentrated. The residue was purified by flash chromatography (petroleum ether/ethyl acetate=2:1) to provide the desired product (900 mg, 49% yield) as a white solid. MS (ESI) m/z=330.2 [M+H]⁺.

### Step 2. Synthesis of tert-butyl ((trans-4-(2-hydroxyethoxy)cyclohexyl)carbamate

To a solution of tert-butyl 2-((trans-4-((tert-butoxycarbonyl)amino)cyclohexyl)oxy)acetate (900 g, 2.73 mmol) in THF (10.0 mL) was added LiAlH₄ (1 M in THF, 3.0 mL, 3.0 mmol) at 0° C. After stirring for 30 min at the same temperature, the reaction was quenched with Na₂SO₄·10H₂O. The mixture was filtered and the filtrate was concentrated under reduced pressure. The residue was purified by flash chromatography (petroleum ether/ethyl acetate=1:1) to provide the desired product (650 mg, 91% yield) as a colorless oil.

### Step 3. Synthesis of tert-butyl 2-(2-((trans-4-((tert-butoxycarbonyl)amino)cyclohexyl)oxy)ethoxy)acetate

To a solution of tert-butyl (trans-4-(2-hydroxyethoxy)cyclohexyl)carbamate (550 mg, 2.12 mmol) in THF (15 mL) was added t-BuOK (285 mg, 2.54 mmol) at 0° C. After stirring at 0° C. for 20 min, a solution of tert-butyl 2-bromoacetate (500 mg, 2.54 mmol) in THF (2.0 mL) was added to the mixture. The resulting mixture was stirred at rt overnight before it was quenched with NH₄Cl solution and extracted with DCM. The combined organic layers were dried over Na₂SO₄, filtered and concentrated. The residue was purified by flash chromatography (petroleum ether/ethyl acetate=2:1) to provide the desired product (500 mg, 63% yield) as a white solid. MS (ESI) m/z=374.2 [M+H]⁺.

The remaining steps were performed according to the procedures for the preparation of BL1-216 to provide the desired product (20 mg, 32% yield over 3 steps) as TFA salt. MS (ESI) m/z=444.4 [M+H]⁺.

### Example 553. 2-(3-(2-(2-Aminoethoxy)ethoxy)propanamido)-N-(5-methyl-1,3,4-thiadiazol-2-yl)benzamide (BL1-227)

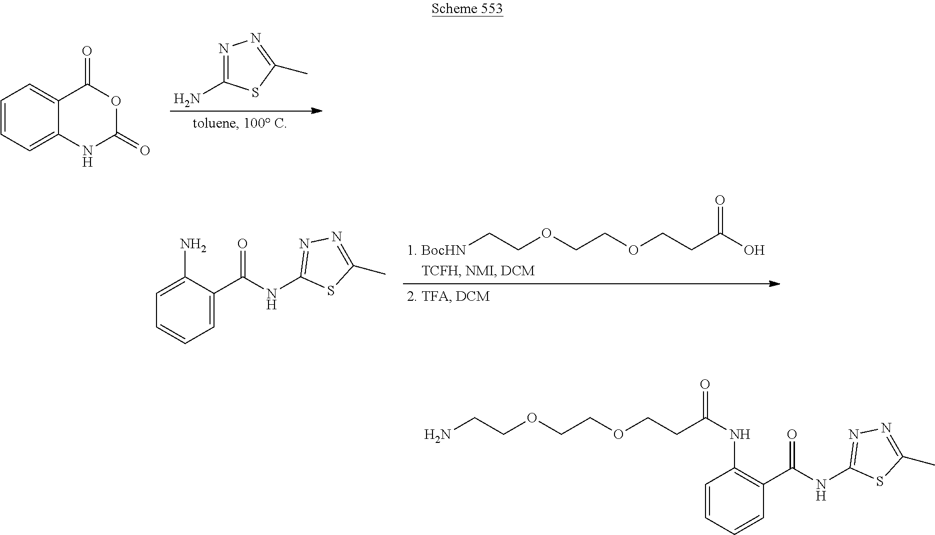

### Step 1. Synthesis of 2-amino-N-(5-methyl-1,3,4-thiadiazol-2-yl)benzamide

The title compound was synthesized following the procedure of step 1 for preparing BL1-213 (340 mg, 78% yield) as a white solid. ¹HNMR (400 MHz, DMSO-d₆) δ 8.50 (brs, 1H), 7.88 (d, J=8.0 Hz 1H), 7.23 (t, J=8.0 Hz, 1H), 6.78 (d, J=8.4 Hz, 1H), 6.56 (t, J=8.4 Hz, 1H), 2.61 (s, 3H). MS (ESI) m/z=235.1 [M+H]⁺.

The remaining steps were performed according to the procedures for preparing BL1-213 to provide the desired product (20 mg, 49% yield over 2 steps) as TFA salt. MS (ESI) m/z=394.6 [M+H]⁺.

### Example 554. 2-((7-Aminoheptyl)amino)-N-(4,5-dimethylthiazol-2-yl)-4-methylbenzamide (BL1-228)

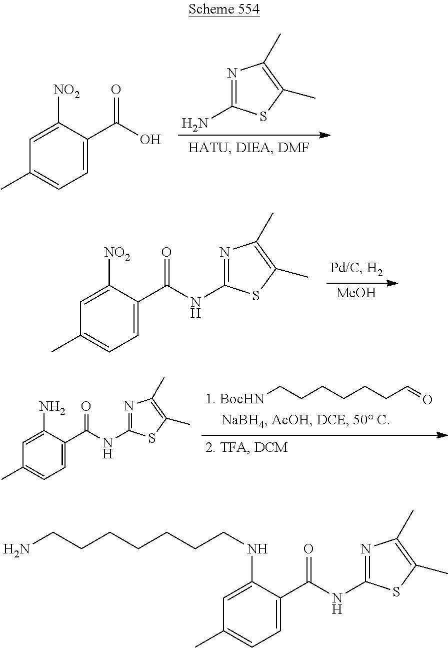

### Step 1. Synthesis of N-(4,5-dimethylthiazol-2-yl)-4-methyl-2-nitrobenzamide

To a solution of 4-methyl-2-nitrobenzoic acid (500 mg, 2.76 mmol) in DMF (20 mL) were added HATU (1.05 g, 2.76 mmol), DIEA (712 mg, 5.52 mmol) and 4,5-dimethylthiazol-2-amine (353.3 g, 2.76 mmol) at rt. The mixture was stirred at rt overnight before it was concentrated under reduced pressure. The residue was purified by silica gel column chromatography (petroleum ether/ethyl acetate=5:1) to provide the desired product (603 mg, 75% yield) as a yellow solid. MS (ESI) m/z=292.0 [M+H]⁺.

### Step 2. Synthesis of 2-amino-N-(4,5-dimethylthiazol-2-yl)-4-methylbenzamide

To a solution of N-(4,5-dimethylthiazol-2-yl)-4-methyl-2-nitrobenzamide (603 mg, 2.07 mmol) in MeOH (10 mL) was added Pd/C (200 mg) at rt. The reaction mixture was stirred at rt under H₂ atmosphere overnight. Then the mixture was filtered and the filtrate was concentrated to provide the desired product (314 mg, 57% yield) as a yellow solid. MS (ESI) m/z=262.1 [M+H]⁺.

### Step 3. Synthesis of tert-butyl (7-((2-((4,5-dimethylthiazol-2-yl)carbamoyl)-5-methylphenyl)amino)heptyl)carbamate

To a stirred solution of 2-amino-N-(4,5-dimethylthiazol-2-yl)-4-methylbenzamide (200 mg, 0.77 mmol) in AcOH (5 mL) and DCE (5 mL) were added tert-butyl (7-oxoheptyl)carbamate (176 mg, 0.77 mmol) and NaBH₄ (146 mg, 3.85 mmol) at rt. The reaction mixture was stirred at 50° C. for 5 h. After cooling down to rt, the reaction was quenched with ice water and extracted with EA (20 ml×3). The combined organic layers were washed with brine (30 mL), dried over sodium sulfate, filtered and concentrated. The residue was purified by silica gel column chromatography (petroleum ether/ethyl acetate=2:1) and prep-HPLC to provide the desired product (101 mg, 28% yield) as a white solid. ¹HNMR (400 MHz, DMSO-d₆) δ 11.92 (brs, 1H), 7.81-7.77 (m, 2H), 6.74-6.73 (m, 1H), 6.54 (s, 1H), 6.39 (d, J=7.6 Hz, 1H), 3.16-3.11 (m, 2H), 2.92-2.89 (m, 2H), 2.32 (s, 3H), 2.25 (s, 3H), 2.18 (s, 3H), 1.61-1.56 (m, 2H), 1.39-1.20 (m, 17H). MS (ESI) m/z=475.1 [M+H]⁺.

### Step 4. Synthesis of 2-((7-aminoheptyl)amino)-N-(4,5-dimethylthiazol-2-yl)-4-methylbenzamide

The title compound was synthesized following the procedure of step 2 for the preparation of BL1-212 to provide the desired product (11 mg, 71% yield) as TFA salt. MS (ESI) m/z=375.4 [M+H]⁺.

### Example 555. 2-(3-(2-(2-Aminoethoxy)ethoxy)propanamido)-N-(6-cyanopyridazin-3-yl)benzamide (BL1-229)

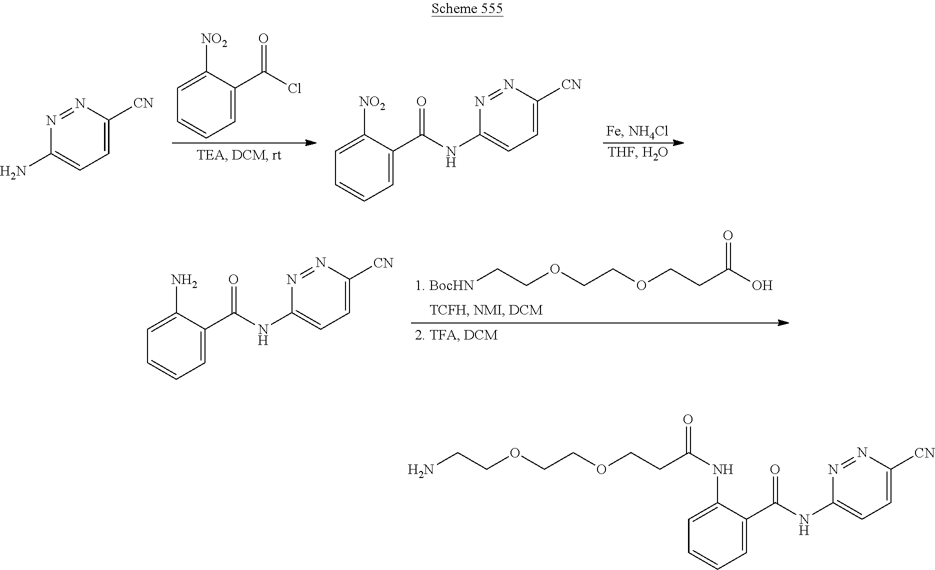

### Step 1. Synthesis of N-(6-cyanopyridazin-3-yl)-2-nitrobenzamide

To a solution of 6-aminopyridazine-3-carbonitrile (200 mg, 1.67 mmol) and TEA (505 mg, 5.0 mmol) in DCM (20 mL) was added a solution of 2-nitrobenzoyl chloride (371 mg, 2.0 mmol) in DCM (3.0 mL) dropwise. After stirring at rt overnight, the reaction mixture was concentrated to get the crude product which was purified by silica gel column chromatography (petroleum ether/ethyl acetate=2:1) to provide the title compound (200.0 mg, 70% yield) as a yellow solid. MS (ESI) m/z=270.2 [M+H]⁺.

### Step 2. Synthesis of 2-amino-N-(6-cyanopyridazin-3-yl)benzamide

To a solution of N-(6-cyanopyridazin-3-yl)-2-nitrobenzamide (200.0 mg, 0.74 mmol) in THF (10.0 mL) and H₂O (3.0 mL) were added iron powder (208 mg, 3.7 mmol) and NH₄Cl (210 mg, 3.7 mmol). The reaction mixture was stirred at rt overnight before it was concentrated under reduced pressure. The residue was diluted with water and extracted with ethyl acetate. The combined organic layers were washed brine, dried over Na₂SO₄, filtered and concentrated. The residue was purified by flash chromatography to provide the desired product (65.0 mg, 52% yield) as a yellow solid. ¹HNMR (400 MHz, DMSO-d₆) δ 11.58 (s, 1H), 8.49 (d, J=9.2 Hz, 1H), 8.29 (d, J=9.6 Hz, 1H), 7.84-7.81 (m, 1H), 7.28-7.23 (m, 1H), 6.79 (d, J=8.0 Hz, 1H), 6.58 (t, J=7.6 Hz, 1H), 6.68-6.48 (m, 2H). MS (ESI) m/z=240.0 [M+H]⁺.

The remaining steps were performed according to the procedures for preparing BL1-213 to provide the desired product (25 mg, 52% yield over 2 steps) as TFA salt. MS (ESI) m/z=399.5 [M+H]⁺.

### Example 556. 2-((7-Aminoheptyl)amino)-N-(4,5-dimethylthiazol-2-yl)-4-(methylamino)benzamide (BL1-230)

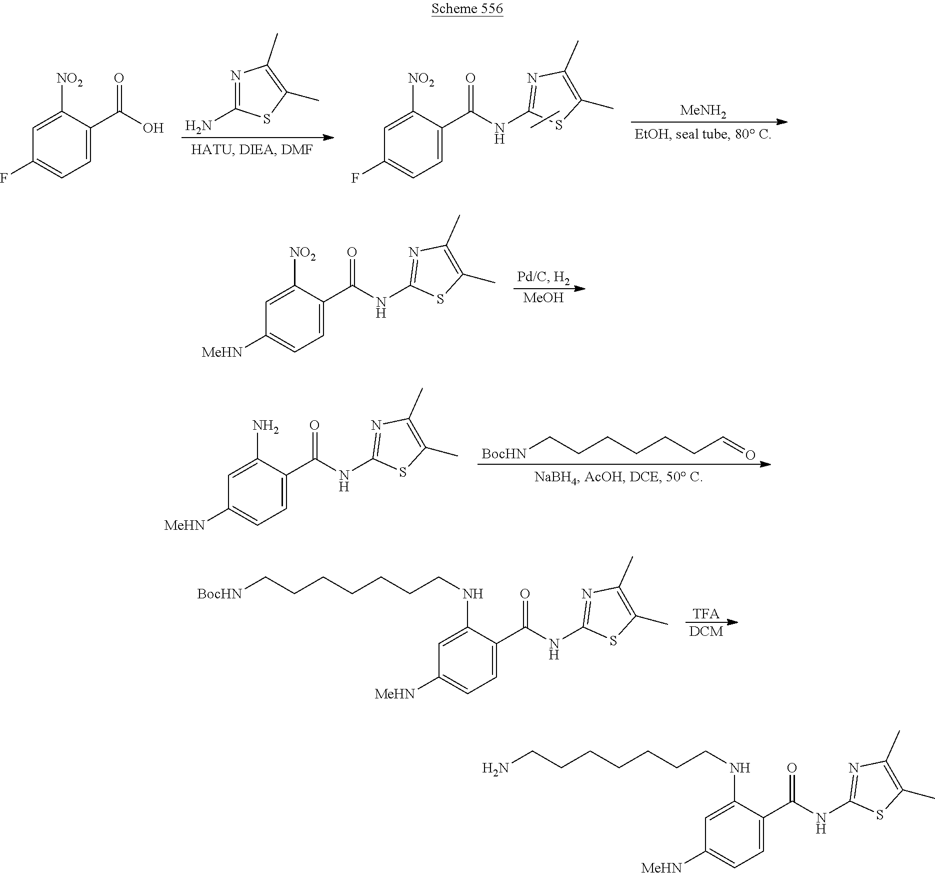

### Step 1. Synthesis of N-(4,5-dimethylthiazol-2-yl)-4-fluoro-2-nitrobenzamide

To a solution of 4-fluoro-2-nitrobenzoic acid (1 g, 5.4 mmol) in DMF (20 mL) were added HATU (2.05 g, 5.4 mmol), DIEA (1.39 g, 10.8 mmol) and 4,5-dimethylthiazol-2-amine (691 mg, 5.4 mmol) at rt. The reaction mixture was stirred at rt for 3 h, before it was concentrated under reduced pressure. The residue was purified by silica gel column chromatography (petroleum ether/ethyl acetate=5:1) to provide the desired product (1.02 g, 64% yield) as a yellow solid. MS (ESI) m/z=296.0 [M+H]⁺.

### Step 2. Synthesis of N-(4,5-dimethylthiazol-2-yl)-4-(methylamino)-2-nitrobenzamide

A solution of N-(4,5-dimethylthiazol-2-yl)-4-fluoro-2-nitrobenzamide (1.02 g, 3.46 mmol) in NH₂Me (1M in EtOH, 40 mL) was stirred at 80° C. in sealed tube overnight. After cooling down to rt, the reaction mixture was poured into water and extracted with dichloromethane (20 mL×4). The organic layers were dried over anhydrous sodium sulfate, filtered and concentrated. The residue was purified by silica gel column chromatography (DCM/MeOH=20:1) to provide the desired product (783 mg, 74% yield) as a yellow solid. MS (ESI) m/z=307.1 [M+H]⁺.

### Step 3. Synthesis of 2-amino-N-(4,5-dimethylthiazol-2-yl)-4-(methylamino)benzamide

To a solution of N-(4,5-dimethylthiazol-2-yl)-4-(methylamino)-2-nitrobenzamide (300 mg, 0.98 mmol) in MeOH (15 mL) was added 10% palladium on charcoal (50 mg) under N2. The suspension was degassed under vacuum and purged with H₂ several times. After stirring at rt under hydrogen atmosphere overnight, the mixture was filtered and the filter cake was washed with MeOH several times. The filtrate was concentrated to afford the desired product (218 mg, crude) as a colorless oil. MS (ESI) m/z=277.1 [M+H]⁺.

### Step 4. Synthesis of tert-butyl (7-((2-((4,5-dimethylthiazol-2-yl)carbamoyl)-5-(methylamino)phenyl)amino)heptyl)carbamate

To a stirred solution of 2-amino-N-(4,5-dimethylthiazol-2-yl)-4-(methylamino)benzamide (150 mg, 0.54 mmol) in AcOH (3 mL) and DCE (3 mL) were added tert-butyl (7-oxoheptyl)carbamate (124 mg, 0.54 mmol) and NaBH₄ (103 mg, 2.7 mmol). The mixture was stirred at 50° C. for 5 h. After cooling down to rt, the reaction was quenched with ice water and extracted with ethyl acetate (20 ml×3). The combined organic layers were washed with brine (30 mL), dried over sodium sulfate, filtered and concentrated. The residue was purified by silica gel column chromatography (DCM/MeOH=20:1) to provide the desired product (158 mg, 59% yield) as a white solid. ¹HNMR (400 MHz, DMSO-d₆) δ 11.41 (s, 1H), 8.18 (s, 1H), 7.72 (d, J=8.4 Hz, 1H), 6.74 (t, J=4.4 Hz, 1H), 6.26-6.25 (m, 1H), 5.83 (dd, J=8.8, 6.8 Hz, 1H), 5.66 (d, J=2.0 Hz, 1H), 3.12-3.07 (m, 2H), 2.92-2.88 (m, 2H), 2.71 (d, J=5.2 Hz, 3H), 2.22 (s, 3H), 2.16 (s, 3H), 1.62-1.57 (m, 2H), 1.39-1.26 (m, 17H). MS (ESI) m/z=490.2 [M+H]⁺.

### Step 5. Synthesis of 2-((7-aminoheptyl)amino)-N-(4,5-dimethylthiazol-2-yl)-4-(methylamino)benzamide

The title compound was synthesized following the procedure of step 2 for the preparation of BL1-212 to provide the desired product (14 mg, 97% yield) as TFA salt. MS (ESI) m/z=390.4 [M+H]⁺.

### Example 557. 2-((5-Aminopentyl)amino)-N-(6-cyclopropyl-5-methylpyridin-2-yl)-4-(methylamino)benzamide (BL1-231)

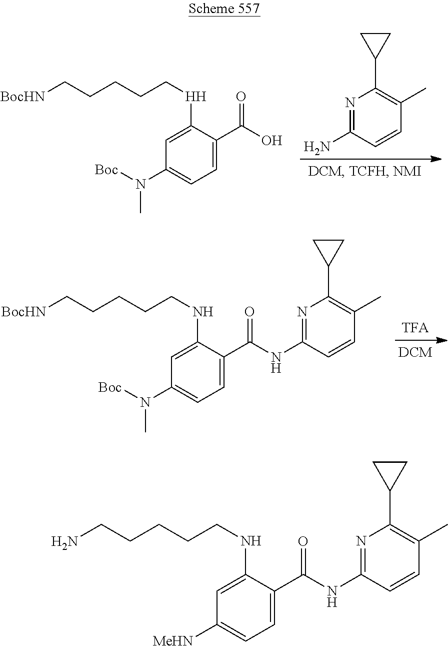

BL1-231 was synthesized following the procedures of steps 2 and 3 for the preparation of BL1-213 (9 mg, 94% yield) as TFA salt. MS (ESI) m/z=382.4 [M+H]⁺.

### Example 558. 3-(2-(2-(3-((2-((4,5-Dimethylthiazol-2-yl)carbamoyl)phenyl)amino)-3-oxopropoxy)ethoxy)ethoxy)propanoic acid (BL1-232)

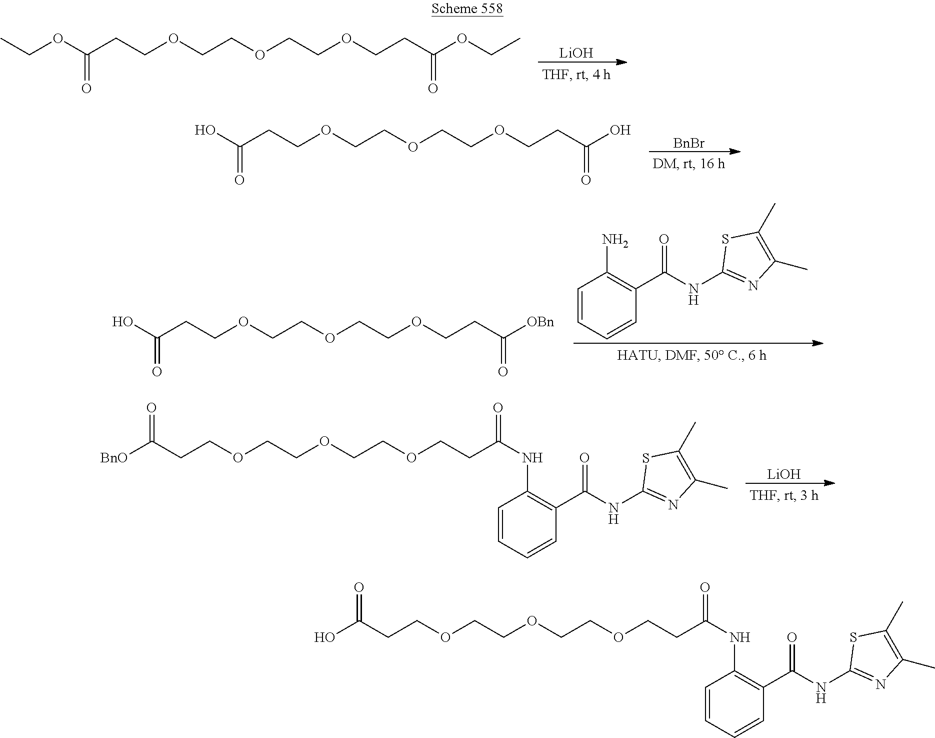

### Step 1. Synthesis of 3,3′-((oxybis(ethane-2,1-diyl))bis(oxy))dipropionic acid

To a solution of diethyl 3,3′-((oxybis(ethane-2,1-diyl))bis(oxy))dipropionate (6.0 g, 19.6 mmol) in THF (40 mL) and H₂O (10 mL) was added LiOH (4.1 g, 171.5 mmol). The mixture was stirred at rt for 4 h, before pH was adjusted to 1-2. The mixture was extracted with EtOAc. The organic layer was concentrated to provide the title compound (1.3 g, 25% yield) as a yellow solid.

### Step 2. Synthesis of 3-oxo-1-phenyl-2,6,9,12-tetraoxapentadecan-15-oic acid

To a solution of 3,3′-((oxybis(ethane-2,1-diyl))bis(oxy))dipropionic acid (1.3 g, 5.2 mmol) in DMF (5 mL) were added DIEA (1.34 g, 10.4 mmol) and BnBr (890 mg, 5.2 mmol). The mixture was stirred at rt for 16 h, before pH was adjusted to 1-2. The mixture was extracted with EtOAc (20 mL×3). The combined organic layers were washed with brine (30 mL), dried over sodium sulfate, filtered and concentrated to provide the title compound (crude, 560 mg, 33% yield) as a yellow oil.

### Step 3. Synthesis of benzyl 3-(2-(2-(3-((2-((4,5-dimethylthiazol-2-yl)carbamoyl)phenyl)amino)-3-oxopropoxy)ethoxy)ethoxy)propanoate

To a solution of 2-amino-N-(4,5-dimethylthiazol-2-yl)benzamide (200 mg, 0.81 mmol) in DMF (4 mL) were added 3-oxo-1-phenyl-2,6,9,12-tetraoxapentadecan-15-oic acid (550 mg, 1.62 mmol), HATU (461 mg, 1.21 mmol) and DIEA (209 mg, 1.62 mmol). The mixture was stirred at 50° C. for 6 h, before it was extracted with EtOAc (20 mL×3). The combined organic layers were washed with brine (30 mL), dried over sodium sulfate, filtered and concentrated. The residue was purified by reverse phase chromatography (0.1% TFA in H₂O and MeCN) to provide the title compound (180 mg, 56% yield) as a yellow oil. MS (ESI) m/z=592.3 [M+H]⁺.

### Step 4. Synthesis of 3-(2-(2-(3-((2-((4,5-dimethylthiazol-2-yl)carbamoyl)phenyl)amino)-3-oxopropoxy)ethoxy)ethoxy)propanoic acid

To a solution of benzyl 3-(2-(2-(3-((2-((4,5-dimethylthiazol-2-yl)carbamoyl)phenyl)amino)-3-oxopropoxy)ethoxy)ethoxy)propanoate (180 mg, 0.32 mmol) in THF (3 mL) and H₂O (1 mL) was added LiOH (66 mg, 1.58 mmol). The mixture was stirred at rt for 3 h, before pH was adjusted to 1-2. The mixture was extracted with EtOAc. The organic layer was concentrated to provide the title compound (100 mg, 62% yield) as a white solid. MS (ESI) m/z=479.8 [M+H]⁺

### Example 559. 7-(3-(2-(2-Aminoethoxy)ethoxy)propanamido)-N-(5-methylpyridin-2-yl)-1,2,3,4-tetrahydroquinoline-6-carboxamide (BL1-233)

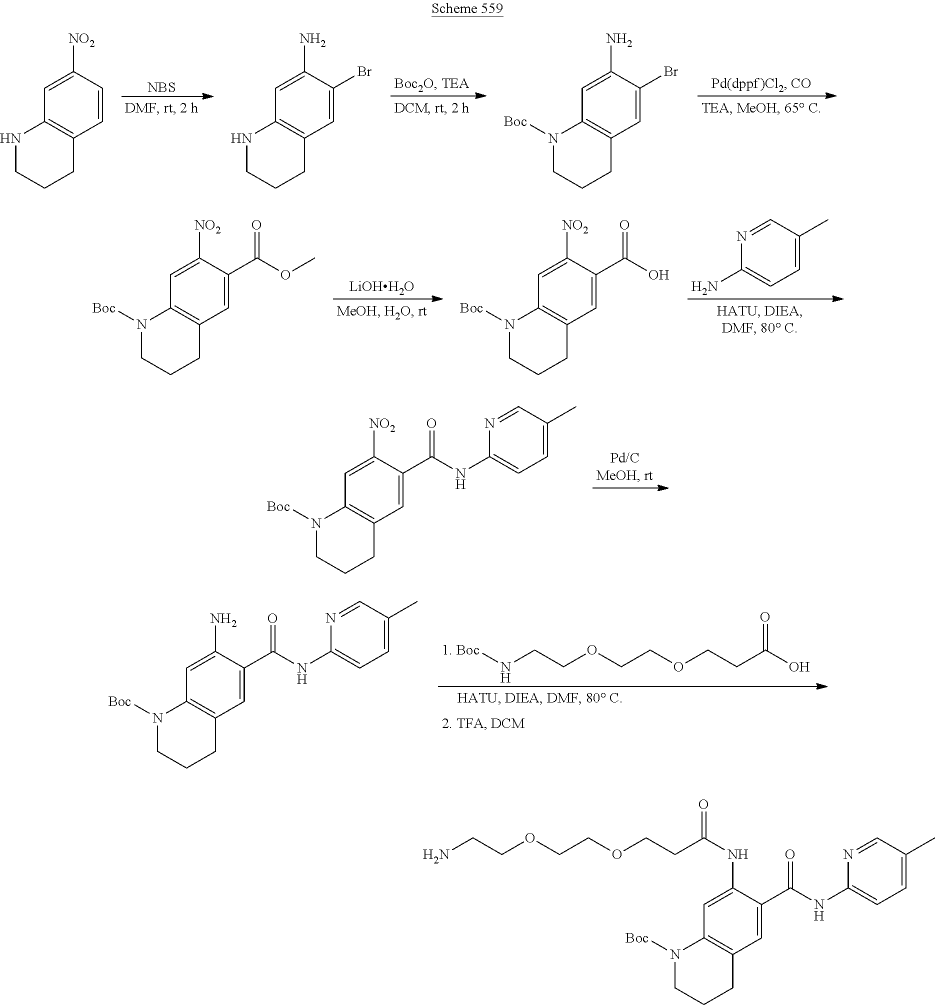

### Step 1. Synthesis of 6-bromo-7-nitro-1,2,3,4-tetrahydroquinoline

A solution of 7-nitro-1,2,3,4-tetrahydroquinoline (10.0 g, 56.2 mmol) and NBS (10.0 g, 56.2 mmol) in DMF (100 mL) was stirred at rt for 2 h, before it was diluted with water (500 mL) and extracted with EtOAc (200 mL×2). The combined organic phase was washed with brine (300 mL×2), dried over Na₂SO₄, filtered and concentrated in vacuo. The residue was purified by silica gel column (petroleum ether/EtOAc=5:1) to provide the title crude compound (10.0 g, 69% yield) as a yellow solid.

### Step 2. Synthesis of tert-butyl 6-bromo-7-nitro-3,4-dihydroquinoline-1(2H)-carboxylate

A solution of 6-bromo-7-nitro-1,2,3,4-tetrahydroquinoline (10.0 g, 38.9 mmol), Boc₂O (17.0 g, 77.8 mmol) and Et₃N (7.86 g, 77.8 mmol) in DCM (100 mL) was stirred at rt for 2 h, before it was concentrated in vacuo. The residue was purified by silica gel chromatography (petroleum ether/EtOAc=5:1) to provide the title compound (11.0 g, 79% yield) as a yellow solid.

### Step 3. Synthesis of 1-(tert-butyl) 6-methyl 7-nitro-3,4-dihydroquinoline-1,6(2H)-dicarboxylate

A solution of tert-butyl 6-bromo-7-nitro-3,4-dihydroquinoline-1(2H)-carboxylate (6.00 g, 16.8 mmol), Pd(dppf)Cl₂ (122 mmol, 0.168 mmol) and Et₃N (3.39 g, 33.6 mmol) in MeOH (60 mL) was stirred at 65° C. overnight under CO atmosphere, before it was cooled to rt. The mixture was diluted with water (300 mL) and extracted with EtOAc (150 mL×2). The combined organic phase was washed with aq. HCl (1 M, 300 mL) and brine (300 mL×2), dried over Na₂SO₄, filtered and concentrated in vacuo. The residue was purified by silica gel chromatography (petroleum ether/EtOAc=5:1) to provide the title compound (2.00 g, 35% yield) as a yellow solid. MS (ESI) m/z=337.1 [M+H]⁺.

### Step 4. Synthesis of 1-(tert-butoxycarbonyl)-7-nitro-1,2,3,4-tetrahydroquinoline-6-carboxylic acid

A solution of 1-(tert-butyl) 6-methyl 7-nitro-3,4-dihydroquinoline-1,6(2H)-dicarboxylate (2.00 g, 5.95 mmol) and LiOH·H₂O (2.50 g, 59.5 mmol) in MeOH (20 mL) and H₂O (5 mL) was stirred at rt for 1 h, before it was diluted with water (100 mL). Aq. HCl (1 M) solution was added to adjust pH to 4. The mixture was extracted with EtOAc (50 mL×2). The combined organic phase was washed brine, dried over Na₂SO₄, filtered and concentrated in vacuo to provide the title compound (1.50 g, 78% yield) as a yellow solid. MS (ESI) m/z=321.2 [M−H]⁻.

### Step 5. Synthesis of tert-butyl 6-((5-methylpyridin-2-yl)carbamoyl)-7-nitro-3,4-dihydroquinoline-1(2H)-carboxylate

A mixture of 1-(tert-butoxycarbonyl)-7-nitro-1,2,3,4-tetrahydroquinoline-6-carboxylic acid (1.50 g, 4.66 mmol), 5-methylpyridin-2-amine (755 mg, 6.99 mmol), HATU (2.66 g, 6.99 mmol) and DIEA (1.20 g, 9.32 mmol) in DMF (15 mL) was stirred at 80° C. for 1 h, before it was cooled to rt. The mixture was diluted with water (100 mL) and extracted with EtOAc (50 mL×2). The combined organic phase was washed with brine, dried over Na₂SO₄, filtered and concentrated in vacuo. The residue was purified by silica gel chromatography (petroleum ether/EtOAc=3:1) to provide the title compound (1.00 g, 52% yield) as a yellow solid. MS (ESI) m/z=413.2 [M+H]⁺.

### Step 6. Synthesis of tert-butyl 7-amino-6-((5-methylpyridin-2-yl)carbamoyl)-3,4-dihydroquinoline-1(2H)-carboxylate

A solution of tert-butyl 6-((5-methylpyridin-2-yl)carbamoyl)-7-nitro-3,4-dihydroquinoline-1(2H)-carboxylate (1.00 g, 2.43 mmol) and Pd/C (10%, 400 mg) in MeOH (20 mL) was stirred at rt for 2 h under hydrogen atmosphere, before it was filtered and concentrated in vacuo. The residue was purified by silica gel chromatography (petroleum ether/EtOAc=3:1) to provide the title compound (500 mg, 54% yield) as a yellow solid. MS (ESI) m/z=383.2 [M+H]⁺.

### Step 7. Synthesis of tert-butyl 7-(2,2-dimethyl-4-oxo-3,8,11-trioxa-5-azatetradecan-14-amido)-6-((5-methylpyridin-2-yl)carbamoyl)-3,4-dihydroquinoline-1(2H)-carboxylate

A solution of tert-butyl 7-amino-6-((5-methylpyridin-2-yl)carbamoyl)-3,4-dihydroquinoline-1(2H)-carboxylate (300 mg, 0.785 mmol), 2,2-dimethyl-4-oxo-3,8,11-trioxa-5-azatetradecan-14-oic acid (217 mg, 0.785 mmol), HATU (447 mg, 1.18 mmol) and DIEA (203 mg, 1.57 mmol) in DMF (3 mL) was stirred at 80° C. for 1 h, before it was cooled to rt. The mixture was diluted with water (30 mL) and extracted with EtOAc (20 mL×2). The combined organic phase was washed with brine, dried over Na₂SO₄, filtered and concentrated in vacuo. The residue was purified by silica gel chromatography (petroleum ether/EtOAc=1:1) to provide the title compound (200 mg, 40% yield) as a yellow solid. MS (ESI) m/z=642.8 [M+H]⁺.

### Step 8. Synthesis of 7-(3-(2-(2-aminoethoxy)ethoxy)propanamido)-N-(5-methylpyridin-2-yl)-1,2,3,4-tetrahydroquinoline-6-carboxamide

A solution of tert-butyl 7-(2,2-dimethyl-4-oxo-3,8,11-trioxa-5-azatetradecan-14-amido)-6-((5-methylpyridin-2-yl)carbamoyl)-3,4-dihydroquinoline-1(2H)-carboxylate (200 mg, 0.312 mmol) in TFA (2 mL) and DCM (2 mL) was stirred at rt for 2 h, before it was directly lyophilized to provide the title compound (TFA salt, 147 mg, 85% yield) as a yellow solid. ¹H NMR (400 MHz, DMSO-d₆) δ 11.43 (s, 1H), 10.30 (s, 1H), 8.20 (d, J=1.2 Hz, 1H), 7.88 (d, J=8.4 Hz, 1H), 7.72 (br s, 3H), 7.69-7.63 (m, 1H), 7.61 (d, J=10.8 Hz, 2H), 3.72-3.69 (m, 4H), 3.58-3.52 (m, 4H), 3.21 (t, J=5.2 Hz, 2H), 2.94-2.88 (m, 2H), 2.64 (t, J=6.0 Hz, 2H), 2.53 (t, J=6.0 Hz, 2H), 2.28 (s, 3H), 1.80-1.77 (m, 2H). MS (ESI) m/z=442.3 [M+H]⁺.

### Example 560. 4-(3-(2-(2-Aminoethoxy)ethoxy)propanamido)-N-(5-methylpyridin-2-yl)-1H-indazole-5-carboxamide (BL1-234)

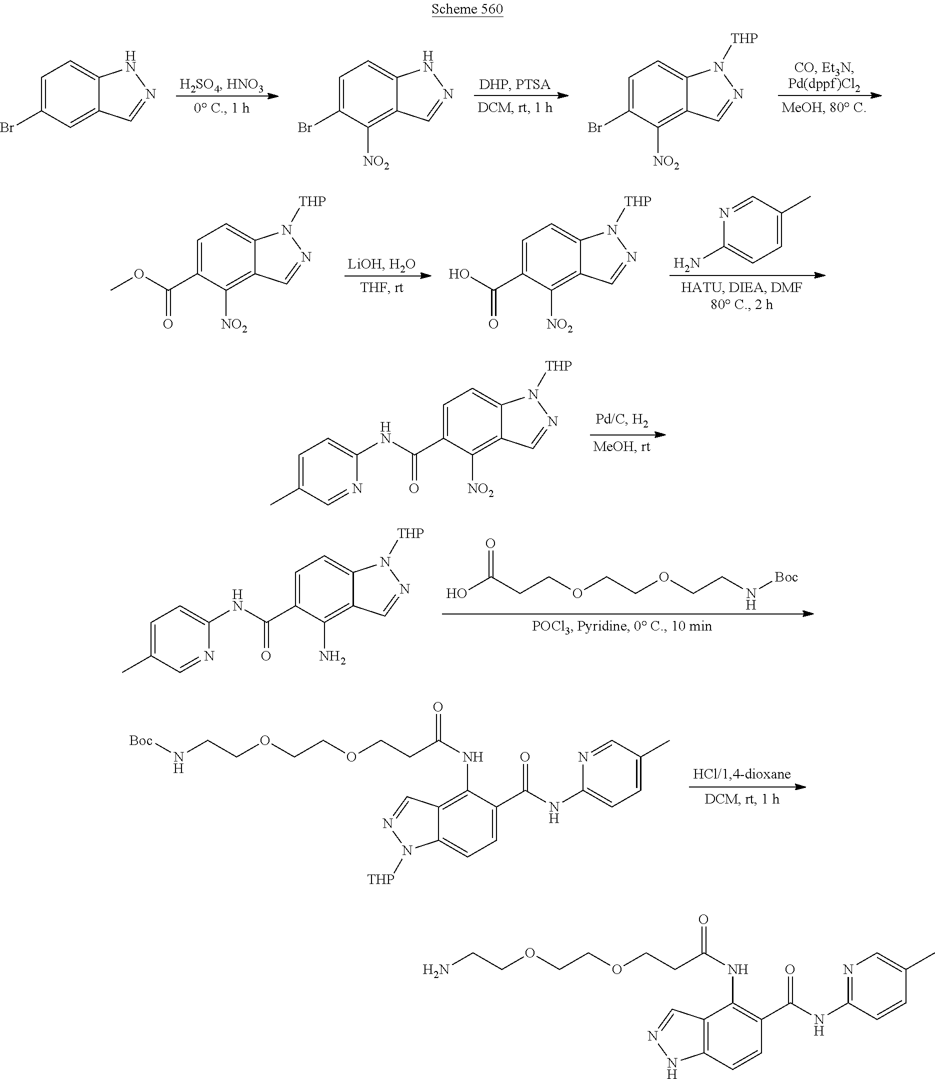

### Step 1. Synthesis of 5-bromo-4-nitro-1H-indazole

A solution of 5-bromo-1H-indazole (20 g, 102 mmol) in conc. sulfuric acid (aq., 98 wt %, 400 mL, 7.6 mol) was cooled to 0° C. Fuming nitric acid (70 wt %, 20 mL, 452 mmol) was added dropwise. The reaction mixture was stirred at 0° C. for 1 h, before it was poured into ice water (900 mL). The precipitate was collected by filtration, washed with water (300 mL) and dried at 50° C. under reduced pressure to provide the title compound (20 g, 82% yield) as a yellow solid. MS (ESI) m/z=242.0 [M+H]⁺.

### Step 2. Synthesis of 5-bromo-4-nitro-1-(tetrahydro-2H-pyran-2-yl)-1H-indazole

To a solution of 5-bromo-4-nitro-1H-indazole (18 g, 74.38 mmol) in DCM (200 mL) was added 3,4-dihydro-2H-pyran (12.5 g, 148.76 mmol) and PTSA (7.06 g, 37.19 mmol). The mixture was stirred at rt for 1 h, before it was concentrated. The residue was diluted with EtOAc (450 mL). The solution was washed with water (200 mL), brine (200 mL), and dried over sodium sulfate. The organic layer was concentrated in vacuo. The residue was purified by silica gel chromatography to provide the title compound (16.2 g, 67% yield) as a colorless liquid. MS (ESI) m/z=326.1 [M+H]⁺.

### Step 3. Synthesis of methyl 4-nitro-1-(tetrahydro-2H-pyran-2-yl)-1H-indazole-5-carboxylate

A solution of 5-bromo-4-nitro-1-(tetrahydro-2H-pyran-2-yl)-1H-indazole (5.0 g, 15.3 mmol), Pd(dppf)Cl₂ (1.12 g, 1.532 mmol), triethylamine (7.71 g, 76.58 mmol) in MeOH (150 mL) was stirred at 80° C. overnight under CO atmosphere (15 psi), before it was cooled to rt. The mixture was concentrated in vacuo. The residue was purified by silica gel chromatography (petroleum ether/EtOAc=4:1) to provide the title compound (2.0 g, 43% yield) as a yellow solid. MS (ESI) m/z=306.1 [M+H]⁺.

### Step 4. Synthesis of 4-nitro-1-(tetrahydro-2H-pyran-2-yl)-1H-indazole-5-carboxylic acid

A solution of methyl 4-nitro-1-(tetrahydro-2H-pyran-2-yl)-1H-indazole-5-carboxylate (2.0 g, 6.55 mmol) and LiOH·H₂O (825 mg, 19.65 mmol) in THF (10 mL) and H₂O (5 mL) was stirred at rt overnight, before it was diluted with water (50 mL). The mixture was acidified to pH=3 with aqueous HCl (1 M) and extracted with EtOAc (50 mL×3). The organic phase was washed with brine, dried over Na₂SO₄, filtered and concentrated in vacuo to provide the title compound (1.8 g, 95% yield) as a light yellow solid. MS (ESI) m/z=292.1 [M+H]⁺.

### Step 5. Synthesis of N-(5-methylpyridin-2-yl)-4-nitro-1-(tetrahydro-2H-pyran-2-yl)-1H-indazole-5-carboxamide

To a solution of 4-nitro-1-(tetrahydro-2H-pyran-2-yl)-1H-indazole-5-carboxylic acid (1.80 g, 6.17 mmol), HATU (2.81 g, 7.40 mmol) and DIPEA (1.59 g, 12.34 mmol) in DMF (20 mL) was added 5-methylpyridin-2-amine (734 mg, 6.79 mmol) at rt. The mixture was stirred at 80° C. for 2 h, before it was cooled to rt. The mixture was poured into water (100 mL) and the solid was filtered. The filter cake was purified by silica gel chromatography (petroleum ether/EtOAc=1:1) to provide the title compound (700 mg, 65% yield) as a light yellow solid. MS (ESI) m/z=382.1 [M+H]⁺.

### Step 6. Synthesis of 4-amino-N-(5-methylpyridin-2-yl)-1-(tetrahydro-2H-pyran-2-yl)-1H-indazole-5-carboxamide

A mixture of N-(5-methylpyridin-2-yl)-4-nitro-1-(tetrahydro-2H-pyran-2-yl)-1H-indazole-5-carboxamide (700 mg, 1.83 mmol) and Pd/C (10 mg) in MeOH (7 mL) was stirred at rt under H₂ (1 atm) overnight. The catalyst was removed by filtration. The filtrate was concentrated under reduced pressure to provide the title compound (400 mg, 62% yield) as an off-white solid. MS (ESI) m/z=352.2 [M+H]⁺.

### Step 7. Synthesis of tert-butyl (2-(2-(3-((5-((5-methylpyridin-2-yl)carbamoyl)-1-(tetrahydro-2H-pyran-2-yl)-1H-indazol-4-yl)amino)-3-oxopropoxy)ethoxy)ethyl)carbamate

To a solution of 4-amino-N-(5-methylpyridin-2-yl)-1-(tetrahydro-2H-pyran-2-yl)-1H-indazole-5-carboxamide (400 mg, 1.13 mmol), and 2,2-dimethyl-4-oxo-3,8,11-trioxa-5-azatetradecan-14-oic acid (631 mg, 2.26 mmol) in pyridine (6 mL) at 0° C. was added POCl₃ (346 mg, 2.26 mmol) dropwise. The mixture was stirred at 0° C. for 10 min, before it was quenched with MeOH (5 mL). The obtained mixture was purified by prep-HPLC to provide the title compound (120 mg, 17% yield) as a light yellow solid. ¹H NMR (400 MHz, DMSO-d₆) δ 10.42 (s, 1H), 10.36 (s, 1H), 8.19-8.18 (m, 1H), 8.09 (s, 1H), 8.05 (d, J=8.4 Hz, 2H), 7.71-7.60 (m, 3H), 6.74-6.72 (m, 1H), 5.89-5.86 (m, 1H), 3.91-3.88 (m, 1H), 3.79-3.73 (m, 1H), 3.66 (t, J=6.4 Hz, 2H), 3.47-3.44 (m, 4H), 3.34-3.33 (m, 2H), 3.05-3.01 (m, 2H), 2.63 (t, J=6.4 Hz, 2H), 2.44-2.39 (m, 1H), 2.27 (s, 3H), 2.07-1.95 (m, 2H), 1.80-1.65 (m, 1H), 1.60-1.52 (m, 2H), 1.37 (s, 9H). MS (ESI) m/z=611.6 [M+H]⁺.

### Step 8. Synthesis of 4-(3-(2-(2-aminoethoxy)ethoxy)propanamido)-N-(5-methylpyridin-2-yl)-1H-indazole-5-carboxamide

To a solution of tert-butyl (2-(2-(3-((5-((5-methylpyridin-2-yl)carbamoyl)-1-(tetrahydro-2H-pyran-2-yl)-1H-indazol-4-yl)amino)-3-oxopropoxy)ethoxy)ethyl)carbamate (13 mg, 21.31 umol) in DCM (0.5 mL) was added HCl (4 M in 1,4-dioxane, 0.2 mL) at rt. The reaction was stirred for 1 h, before it was concentrated. The solid was collected by filtration and dried in vacuo to provide the title compound (7.9 mg, 88% yield) as a yellow solid. MS (ESI) m/z=427.3 [M+H]⁺.

### Example 561. 6-(3-(2-(2-Aminoethoxy)ethoxy)propanamido)-N-(5-methylpyridin-2-yl)indoline-5-carboxamide (BL1-235)

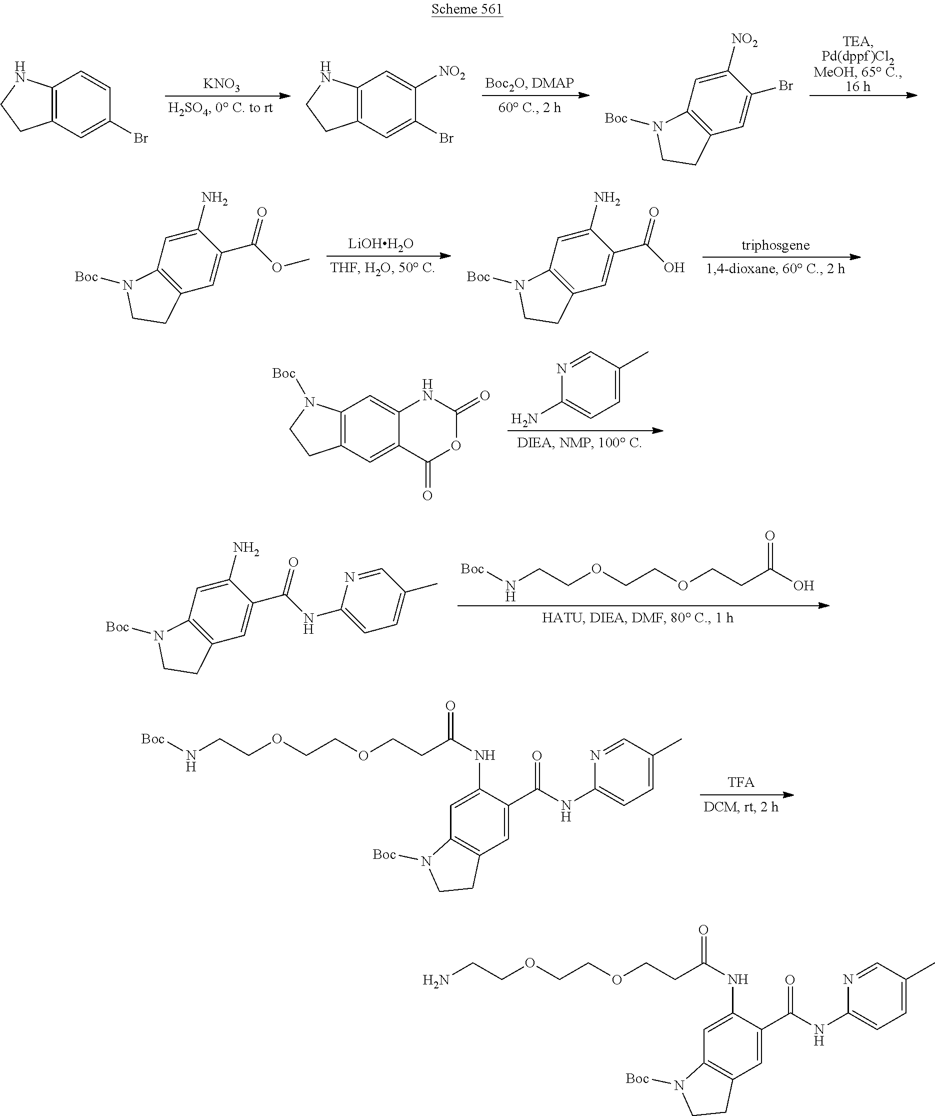

### Step 1. Synthesis of 5-bromo-6-nitroindoline

To a solution of 5-bromoindoline (30.0 g, 152 mmol) in conc. H₂SO₄ (150 mL) at 0° C. was added KNO₃ (15.3 g, 152 mmol). The mixture was stirred at rt for 4 h, before it was poured into ice water (600 mL) and extracted with EtOAc (200 mL×2). The combined organic phase was washed with brine, dried over Na₂SO₄, filtered and concentrated in vacuo. The residue was purified by silica gel chromatography (petroleum ether/EtOAc=5:1) to provide the title compound (30.0 g, 81% yield) as a red solid. MS (ESI) m/z=245.0 [M+H]⁺.

### Step 2-4. Synthesis of 6-amino-1-(tert-butoxycarbonyl)indoline-5-carboxylic acid

The title compound was synthesized following the standard procedures for steps 2 to 4 of the preparation of BL1-233 (800 mg, 4% yield over 3 steps) as a brown solid. MS (ESI) m/z=279.2 [M+H]⁺.

### Step 5. Synthesis of tert-butyl 2,4-dioxo-1,4,6,7-tetrahydro-[1,3]oxazino[5,4-f]indole-8(2H)-carboxylate

A solution of 6-amino-1-(tert-butoxycarbonyl)indoline-5-carboxylic acid (800 mg, 2.88 mmol) and triphosgene (284 mg, 0.958 mmol) in 1,4-dioxane (8 mL) was stirred at 60° C. for 2 h, before it was cooled to rt. The mixture was filtered. The filter cake was washed with THF (20 mL), and dried in vacuo to provide the title compound (600 mg, 69% yield) as a white solid. MS (ESI) m/z=346.1 [M+H+MeCN]⁺.

### Step 6. Synthesis of tert-butyl 6-amino-5-((5-methylpyridin-2-yl)carbamoyl)indoline-1-carboxylate

A solution of tert-butyl 2,4-dioxo-1,4,6,7-tetrahydro-[1,3]oxazino[5,4-f]indole-8(2H)-carboxylate (600 mg, 1.97 mmol), 5-methylpyridin-2-amine (426 mg, 3.94 mmol) and DIEA (496 mg, 3.94 mmol) in NMP (6 mL) was stirred at 100° C. overnight, before it was cooled to rt. The mixture was diluted with water (30 mL) and extracted with EtOAc (30 mL×2). The combined organic phase was washed with brine, dried over Na₂SO₄, filtered and concentrated in vacuo. The residue was purified by silica gel chromatography (petroleum ether/EtOAc=2:1) to provide the title compound (300 mg, 41% yield) as a yellow solid. MS (ESI) m/z=369.2 [M+H]⁺.

### Step 7-8. Synthesis of 6-(3-(2-(2-aminoethoxy)ethoxy)propanamido)-N-(5-methylpyridin-2-yl)indoline-5-carboxamide

BL1-235 was synthesized following the standard procedures of steps 7-8 for the preparation of BL1-233 (TFA salt, 160 mg, 36% yield over 2 steps) as a yellow solid. ¹H NMR (400 MHz, DMSO-d₆) δ 11.53 (s, 1H), 10.40 (s, 1H), 8.21 (s, 1H), 7.87 (d, J=8.4 Hz, 1H), 7.72-7.65 (m, 6H), 3.70 (t, J=6.0 Hz, 2H), 3.60-3.53 (m, 8H), 2.97-2.90 (m, 4H), 2.55 (t, J=6.0 Hz, 2H), 2.29 (s, 3H). MS (ESI) m/z=428.2 [M+H]⁺.

### Example 562. 4-(3-(2-(2-Aminoethoxy)ethoxy)propanamido)-N-(5-methylpyrimidin-2-yl)-1-tosyl-1H-indole-5-carboxamide (BL1-236)

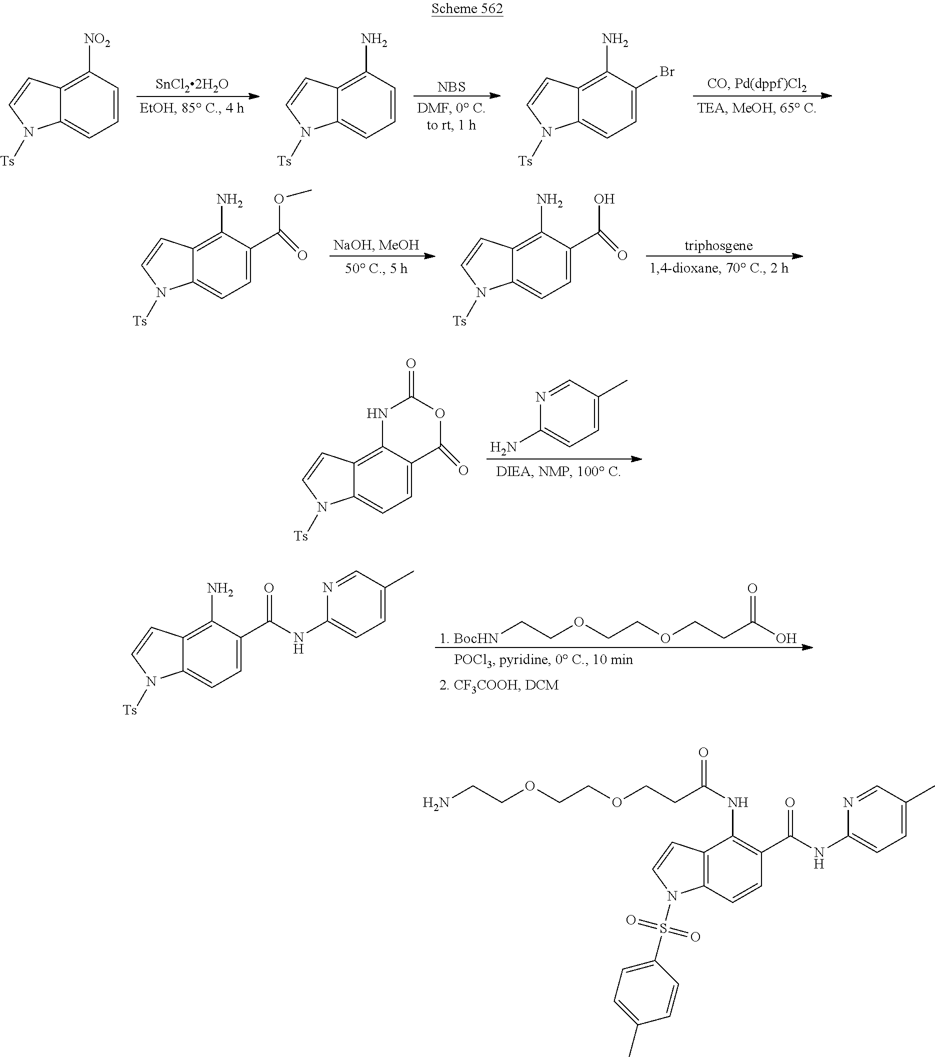

### Step 1. Synthesis of 1-tosyl-1H-indol-4-amine

A solution of 4-nitro-1-tosyl-1H-indole (24.0 g, 75.9 mmol), SnCl₂·2H₂O (68.5 g, 303.6 mmol) in EtOH (400 mL) was stirred at 85° C. for 4 h, before it was cooled to rt. The mixture was concentrated in vacuo. The residue was diluted with water (1 L) and extracted with DCM (500 mL×3). The organic phase was washed with brine, dried over Na₂SO₄, filtered and concentrated in vacuo. The crude product was purified by silica gel chromatography (petroleum ether/EtOAc=3:1) to provide the title compound (20.0 g, 92% yield) as a white solid. MS (ESI) m/z=287.1 [M+H]⁺.

### Step 2. Synthesis of 5-bromo-1-tosyl-1H-indol-4-amine

To a solution of 1-tosyl-1H-indol-4-amine (2.00 g, 6.99 mmol) in DMF (20 mL) at 0° C. was added NBS (1.22 g, 6.85 mmol). The mixture was stirred at rt for 1 h, before it was diluted with water (100 mL) and extracted with EtOAc (50 mL×3). The organic phase was washed with brine, dried over Na₂SO₄, filtered and concentrated in vacuo. The residue was purified by silica gel chromatography (petroleum ether/EtOAc=3:1) to provide the title compound (1.00 g, 39% yield) as a white solid. MS (ESI) m/z=365.1 [M+H]⁺.

### Step 3. Synthesis of methyl 4-amino-1-tosyl-1H-indole-5-carboxylate

A solution of 5-bromo-1-tosyl-1H-indol-4-amine (1.00 g, 2.74 mmol), Pd(dppf)Cl₂ (200 mg, 0.274 mmol) and triethylamine (1.38 g, 13.7 mmol) in MeOH (15 mL) was stirred at 65° C. overnight under CO atmosphere (15 psi), before it was cooled to rt, and concentrated in vacuo. The residue was purified by silica gel chromatography (petroleum ether/EtOAc=4:1) to provide the title compound (crude, 400 mg) as a yellow solid. MS (ESI) m/z=345.1 [M+H]⁺.

### Step 4. Synthesis of 4-amino-1-tosyl-1H-indole-5-carboxylic acid

A solution of methyl 4-amino-1-tosyl-1H-indole-5-carboxylate (crude, 400 mg) and NaOH (232 mg, 5.80 mmol) in MeOH (10 mL) and H₂O (5 mL) was stirred at 50° C. for 5 h, before it was diluted with water (50 mL). The mixture was acidified to pH=3 with aqueous HCl (1 M) and extracted with EtOAc (50 mL×3). The organic phase was washed with brine, dried over Na₂SO₄, filtered and concentrated in vacuo. The residue was purified by prep-HPLC to provide the title compound (100 mg, 11% yield over two steps) as an off-yellow solid. MS (ESI) m/z=331.2 [M+H]⁺.

### Step 5. Synthesis of 7-tosyl-1,7-dihydro-[1,3]oxazino[4,5-e]indole-2,4-dione

A solution of 4-amino-1-tosyl-1H-indole-5-carboxylic acid (1.00 g, 3.03 mmol), triphosgene (300 mg, 1.01 mmol) in 1,4-dioxane (10 mL) was stirred at 70° C. for 2 h, before it was cooled to rt. The solid was filtered and the filter cake was washed with petroleum ether (50 mL) to provide the title compound (700 mg, 65% yield) as an off-yellow solid. MS (ESI) m/z=357.1 [M+H]⁺.

### Step 6. Synthesis of 4-amino-N-(5-methylpyridin-2-yl)-1-tosyl-1H-indole-5-carboxamide

A solution of 7-tosyl-1,7-dihydro-[1,3]oxazino[4,5-e]indole-2,4-dione (700 mg, 1.97 mmol), 5-methylpyridin-2-amine (425 mg, 3.94 mmol) and DIEA (1.02 g, 7.88 mmol) in NMP (7 mL) was stirred at 100° C. overnight, before it was cooled to rt. The mixture was diluted with water (50 mL) and extracted with EtOAc (50 mL×3). The organic phase was washed with brine, dried over Na₂SO₄, filtered and concentrated in vacuo. The residue was purified by silica gel chromatography (petroleum ether/EtOAc=2:1) to provide the title compound (180 mg, 22% yield) as an off-yellow solid. MS (ESI) m/z=421.2 [M+H]⁺.

### Step 7. Synthesis of tert-butyl (2-(2-(3-((5-((5-methylpyridin-2-yl)carbamoyl)-1-tosyl-1H-indol-4-yl)amino)-3-oxopropoxy)ethoxy)ethyl)carbamate

To a solution of 4-amino-N-(5-methylpyridin-2-yl)-1-tosyl-1H-indole-5-carboxamide (180 mg, 0.429 mmol), 2,2-dimethyl-4-oxo-3,8,11-trioxa-5-azatetradecan-14-oic acid (238 mg, 0.858 mmol) in pyridine (3 mL) at 0° C. was added POCl₃ (131 mg, 0.858 mmol) dropwise. The mixture was stirred at 0° C. for 10 min, before it was quenched with MeOH (2 mL). The result mixture was purified by prep-HPLC to provide the title compound (60 mg, 21% yield) as an off-yellow solid. ¹H NMR (400 MHz, DMSO-d₆) δ 10.30 (s, 1H), 9.98 (s, 1H), 8.15-8.14 (m, 1H), 8.01 (d, J=8.4 Hz, 1H), 7.90 (d, J=8.4 Hz, 2H), 7.87-7.84 (m, 2H), 7.64 (dd, J=8.4 Hz, 2.0 Hz, 1H), 7.59 (d, J=8.8 Hz, 1H), 7.41 (d, J=8.4 Hz, 2H), 6.81 (d, J=4.0 Hz, 1H), 6.75 (t, J=1.6 Hz, 1H), 3.60 (t, J=6.4 Hz, 2H), 3.44-3.42 (m, 4H), 3.38-3.32 (m, 2H), 3.05-3.01 (m, 2H), 2.55 (t, J=6.4 Hz, 2H), 2.33 (s, 3H), 2.26 (s, 3H), 1.37 (s, 9H). MS (ESI) m/z=680.3 [M+H]⁺.

### Step 8. Synthesis of 4-(3-(2-(2-aminoethoxy)ethoxy)propanamido)-N-(5-methylpyrimidin-2-yl)-1-tosyl-1H-indole-5-carboxamide

BL1-236 was synthesized following the standard procedure for step 8 of the preparation of BL1-233 (TFA salt, 62 mg, 98% yield) as a yellow solid. MS (ESI) m/z=580.2 [M+H]⁺.

### Example 563. 2-((5-Aminopentyl)amino)-N-(6-(dimethylamino)pyridazin-3-yl)-4-methylbenzamide (BL1-237)

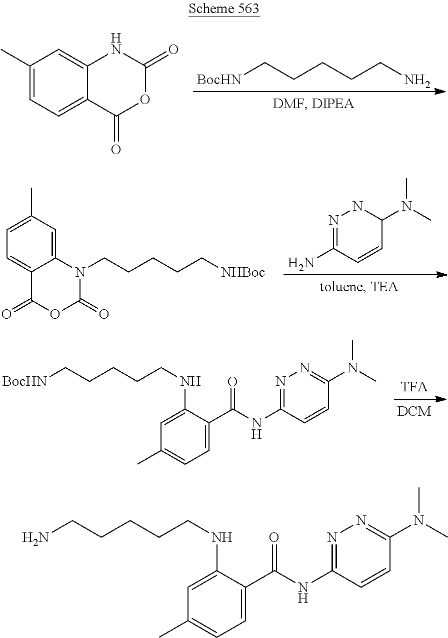

BL1-237 was synthesized following the procedures for preparing BL1-238 (40 mg, 7% yield over 3 steps) as TFA salt. MS (ESI) m/z=357.4 [M+H]⁺.

### Example 564. 2-((5-Aminopentyl)amino)-N-(6-(dimethylamino)pyridazin-3-yl)-4-fluorobenzamide (BL1-238)

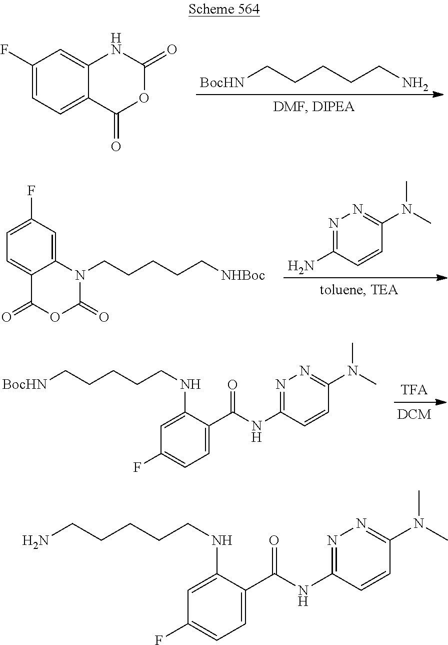

### Step 1. Synthesis of tert-butyl (5-(7-fluoro-2,4-dioxo-2H-benzo[d][1,3]oxazin-1(4H)-yl)pentyl)carbamate

To a solution of 7-fluoro-2H-benzo[d][1,3]oxazine-2,4(1H)-dione (200 mg, 1.1 mmol) and DIPEA (428 mg, 3.3 mmol) in DMF (5 mL) was added tert-butyl (5-bromopentyl)carbamate (352 mg, 1.3 mmol) at rt. The reaction mixture was stirred at 70° C. for 16 h. After cooling down to rt, the reaction was quenched with water (30 mL) and extracted with ethyl acetate (20 mL×3). The combined organic layers were washed with brine, dried over anhydrous sodium sulfate, filtered and concentrated to give the crude product (85 mg, 21% yield) as a colorless oil. MS (ESI) m/z=267.2 [M-100+H]⁺.

### Step 2. Synthesis of tert-butyl (5-((2-((6-(dimethylamino)pyridazin-3-yl)carbamoyl)-5-fluorophenyl)amino)pentyl)carbamate

To a solution of tert-butyl (5-(7-fluoro-2,4-dioxo-2H-benzo[d][1,3]oxazin-1(4H)-yl)pentyl)carbamate (70 mg, 0.19 mmol) in toluene (10 mL) were added TEA (57 mg, 0.57 mmol) and N³,N³-dimethylpyridazine-3,6-diamine (31 mg, 0.22 mmol) at rt. After stirring at 100° C. for 16 h, the solution was concentrated under reduced pressure. The resulting residue was purified by silica gel flash chromatography to provide the desired product (45 mg, 51% yield) as a yellow oil. MS (ESI) m/z=461.5 [M+H]⁺.

### Step 3. Synthesis of 2-((5-aminopentyl)amino)-N-(6-(dimethylamino)pyridazin-3-yl)-4-fluorobenzamide

To a solution of tert-butyl (5-((2-((6-(dimethylamino)pyridazin-3-yl)carbamoyl)-5-fluorophenyl)amino)pentyl)carbamate (45 mg, 0.09 mmol) in DCM (2 mL) was added TFA (1 mL) at 0° C. After the reaction mixture was stirred at rt for 1 h, it was concentrated under reduced pressure to provide the desired product (35 mg, 75% yield) as TFA salt. MS (ESI) m/z=361.4 [M+H]⁺.

### Example 565. 2-((5-aminopentyl)amino)-4-chloro-N-(6-(dimethylamino)pyridazin-3-yl)benzamide (BL1-239)

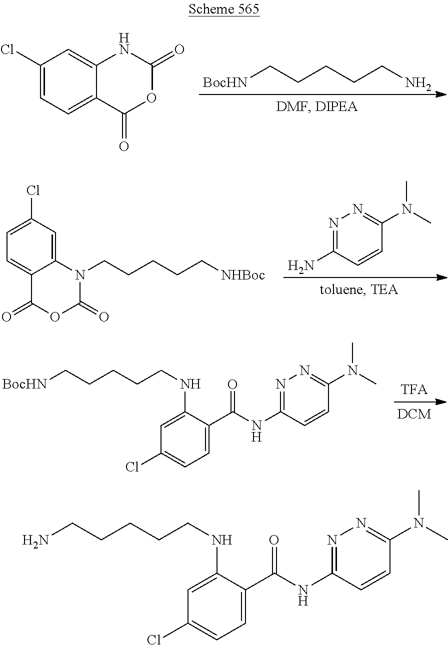

BL1-239 was synthesized following the procedures for preparing BL1-238 (42 mg, 18% yield over 3 steps) as TFA salt. MS (ESI) m/z=377.3 [M+H]⁺.

### Example 566. 5-(3-(2-(2-Aminoethoxy)ethoxy)propanamido)-N-(5-methylpyridin-2-yl)quinoline-6-carboxamide (BL1-240)

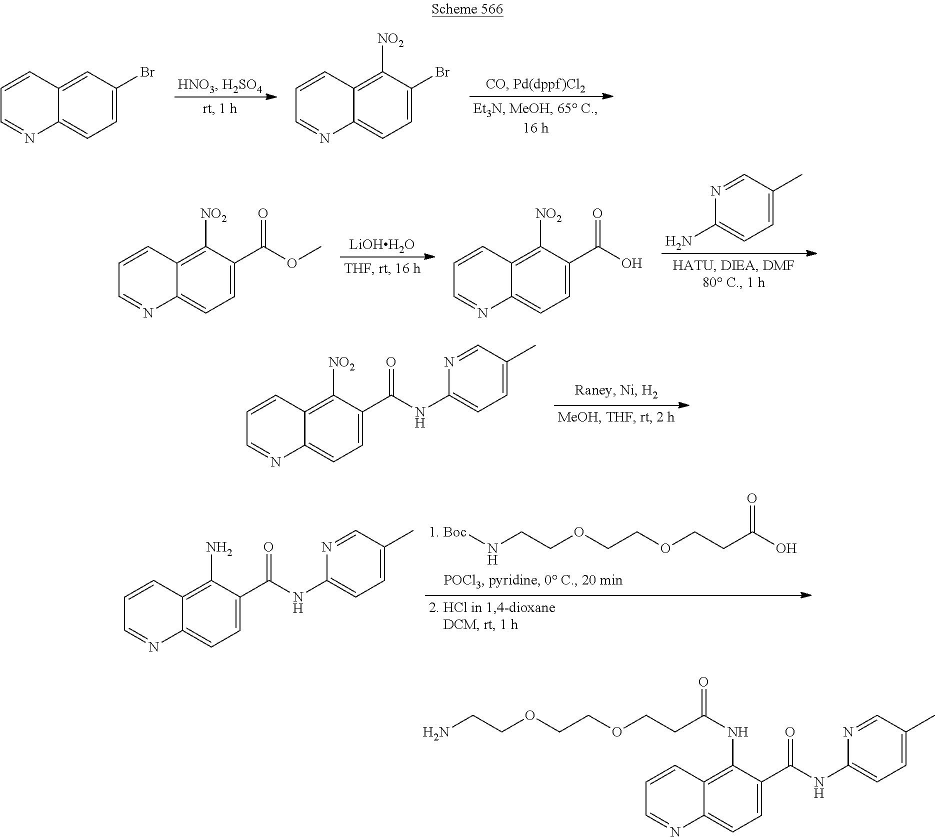

### Step 1. Synthesis of 6-bromo-5-nitroquinoline

To a solution of 6-bromoquinoline (20.0 g, 96.1 mmol) in conc. H₂SO₄ (50 mL) was added conc. HNO₃ (8 mL) at 0° C. The mixture was stirred at rt for 1 h, before it was diluted with ice water (200 mL). The mixture was acidified to pH=7 with aq. NaOH (4.5 M). The precipitate was filtered and the solid was dried in vacuo to provide the title compound (20.0 g, 82% yield) as a white solid. MS (ESI) m/z=253.0 [M+H]⁺.

### Step 2-4. Synthesis of N-(5-methylpyridin-2-yl)-5-nitroquinoline-6-carboxamide

The title compound was synthesized following the standard procedures of step 3-5 for the preparation of BL1-234 (1.40 g, 6% yield over 3 steps) as a pale yellow solid. MS (ESI) m/z=309.0 [M+H]⁺.

### Step 5. Synthesis of methyl 5-amino-N-(5-methylpyridin-2-yl)quinoline-6-carboxamide

A mixture of N-(5-methylpyridin-2-yl)-5-nitroquinoline-6-carboxamide (1.40 g, 4.54 mmol), and Raney Ni (800 mg, 13.6 mmol) in MeOH (10 mL) and THF (10 mL) was stirred at rt for 2 h under H₂ atmosphere. The mixture was filtered and concentrated in vacuo to provide the compound (260 mg, 21% yield) as a white solid. MS (ESI) m/z=396.3 [M+H]⁺.

### Step 6. Synthesis of tert-butyl (2-(2-(3-((6-((5-methylpyridin-2-yl)carbamoyl)quinolin-5-yl)amino)-3-oxopropoxy)ethoxy)ethyl)carbamate

To a solution of methyl 5-amino-N-(5-methylpyridin-2-yl)quinoline-6-carboxamide (260 mg, 0.934 mmol), and 2,2-dimethyl-4-oxo-3,8,11-trioxa-5-azatetradecan-14-oic acid (518 mg, 1.87 mmol) in pyridine (2 mL) was added POCl₃ (290 mg, 1.87 mmol). The reaction mixture was stirred at 0° C. for 20 min, before it was purified by prep-HPLC to provide the title compound (70 mg, 14% yield) as a white solid. ¹H NMR (400 MHz, DMSO-d₆) δ 10.39 (s, 1H), 10.11 (s, 1H), 8.99 (d, J=2.8 Hz, 1H), 8.48 (d, J=8.4 Hz, 1H), 8.19 (s, 1H), 8.09 (d, J=8.0 Hz, 1H), 8.03 (d, J=8.4 Hz, 1H), 7.93 (d, J=8.8 Hz, 1H), 7.69-7.63 (m, 2H), 6.75 (t, J=5.2 Hz, 1H), 3.66 (t, J=6.4 Hz, 2H), 3.51-3.44 (m, 6H), 3.07-3.03 (m, 2H), 2.65 (t, J=6.0 Hz, 2H), 2.29 (s, 3H), 1.39 (s, 9H). MS (ESI) m/z=538.5 [M+H]⁺.

### Step 7. Synthesis of 4-(3-(2-(2-aminoethoxy)ethoxy)propanamido)-N-(5-methylpyridin-2-yl)-1H-indazole-5-carboxamide

To a solution of tert-butyl (2-(2-(3-((6-((5-methylpyridin-2-yl)carbamoyl)quinolin-5-yl)amino)-3-oxopropoxy)ethoxy)ethyl)carbamate (10 mg, 18.60 umol) in DCM (0.5 mL) was added HCl (4 M in 1,4-dioxane, 0.2 mL) at rt. The reaction mixture was stirred for 1 h, before it was concentrated to remove DCM and 1,4-dioxane. The resulting solid was collected by filtration and dried in vacuo to provide (5.5 mg, 67% yield) as a yellow solid. MS (ESI) m/z=438.21 [M+H]⁺.

### Example 567. 2-(10-Aminodecanamido)-N-(4,5-dimethylthiazol-2-yl)benzamide (BL1-241)

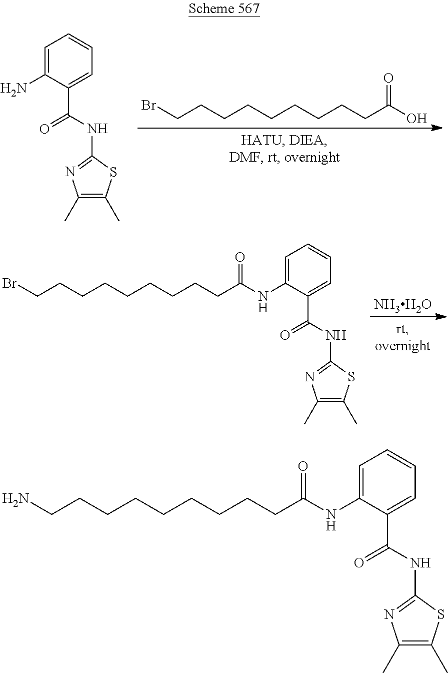 [SMILES: Cc1nc(NC(=O)c2ccccc2N)sc1C ; O=C(O)CCCCCCCCCBr ; Cc1nc(NC(=O)c2ccccc2NC(=O)CCCCCCCCCBr)sc1C ; "NH3·H2O" ; Cc1nc(NC(=O)c2ccccc2NC(=O)CCCCCCCCCN)sc1C]

### Step 1. Synthesis of 2-(10-bromodecanamido)-N-(4,5-dimethylthiazol-2-yl)benzamide

To a solution of 2-amino-N-(4,5-dimethylthiazol-2-yl) benzamide (150 mg, 0.6 mmol) and 10-bromodecanoic acid (150 mg, 0.6 mmol) in DMF (4 mL) were added HATU (342 mg, 0.9 mmol) and DIEA (232 mg, 1.8 mmol). The reaction mixture stirred at rt overnight before it was quenched with water. The mixture was extracted with EtOAc, washed with water and brine, dried over sodium sulfate, filtered and concentrated to provide the title compound (300 mg, 100% yield) as a white solid. MS (ESI) m/z=480.4 [M−H]⁻.

### Step 2. Synthesis of 2-(10-aminodecanamido)-N-(4,5-dimethylthiazol-2-yl)benzamide

A solution of 2-(10-bromodecanamido)-N-(4,5-dimethylthiazol-2-yl)benzamide (300 mg, 0.62 mmol) in NH₄OH (5 ml) was stirred at rt overnight, before it was purified by reverse-phase chromatography to provide the title compound (130 mg, 51% yield) as a yellow oil. MS (ESI) m/z=417.2 [M+H]⁺.

### Example 568. 3-((2-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)ethyl)amino)-N-(6-methoxypyridazin-3-yl)-2-methylbenzamide (CPD-341)

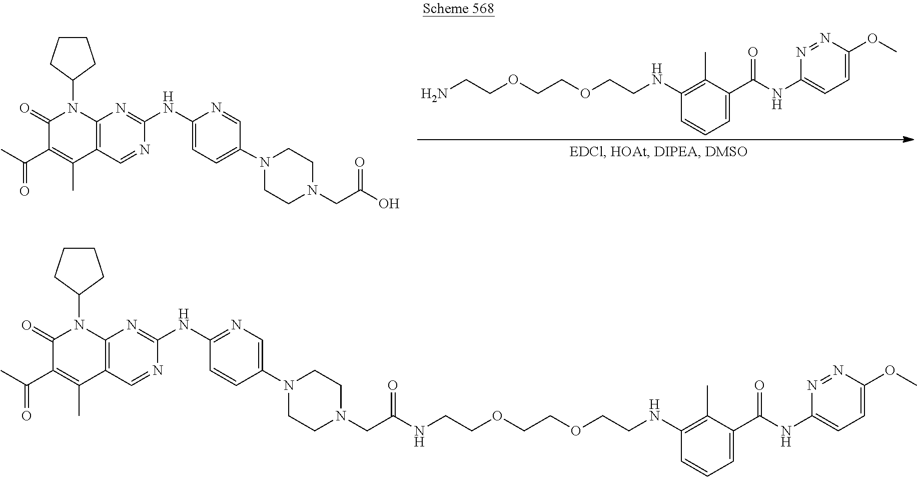 [SMILES: CC(=O)c1c(C)c2cnc(Nc3ccc(N4CCN(CC(=O)O)CC4)cn3)nc2n(C2CCCC2)c1=O ; COc1ccc(NC(=O)c2cccc(NCCOCCOCCN)c2C)nn1 ; COc1ccc(NC(=O)c2cccc(NCCOCCOCCNC(=O)CN3CCN(c4ccc(Nc5ncc6c(C)c(C(C)=O)c(=O)n(C7CCCC7)c6n5)nc4)CC3)c2C)nn1]

To a mixture of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid (15.1 mg, 0.030 mmol) and 3-((2-(2-(2-aminoethoxy)ethoxy)ethyl)amino)-N-(6-methoxypyridazin-3-yl)-2-methylbenzamide (15 mg, 0.03 mmol) in DMSO (2 mL) were added HOAT (6.1 mg, 0.045 mmol), EDCI (8.6 mg, 0.045 mmol) and DIPEA (19.3 mg, 0.15 mmol) at rt. After the reaction mixture was stirred at rt for 16 h, it was purified by reverse-phase chromatography to provide the desired product (14.3 mg, 48% yield) as a yellow solid. MS (ESI) m/z=877.7 [M+H]⁺.

### Example 569. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methoxypyridin-2-yl)benzamide (CPD-342)

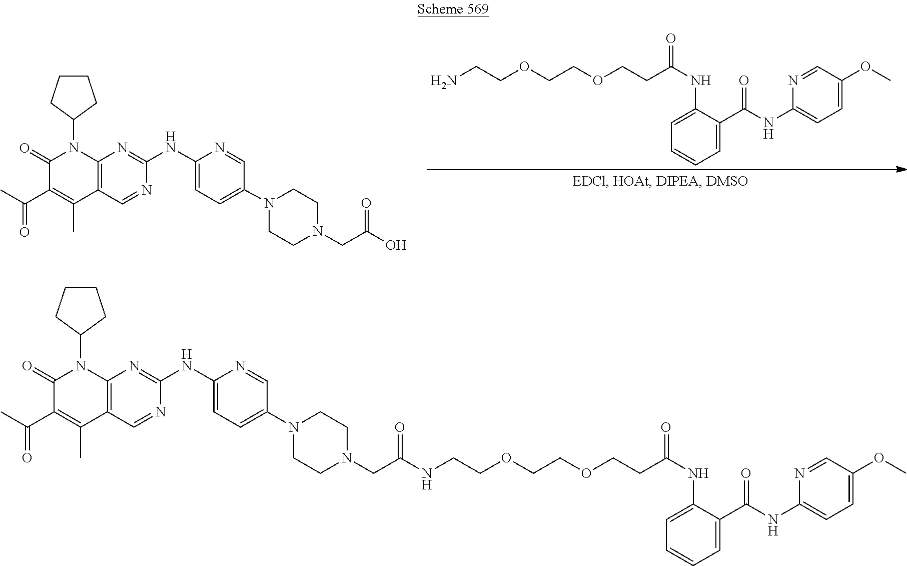 [SMILES: CC(=O)c1c(C)c2cnc(Nc3ccc(N4CCN(CC(=O)O)CC4)cn3)nc2n(C2CCCC2)c1=O ; COc1ccc(NC(=O)c2ccccc2NC(=O)CCOCCOCCN)nc1 ; COc1ccc(NC(=O)c2ccccc2NC(=O)CCOCCOCCNC(=O)CN2CCN(c3ccc(Nc4ncc5c(C)c(C(C)=O)c(=O)n(C6CCCC6)c5n4)nc3)CC2)nc1]

CPD-342 was synthesized following the standard procedure for preparing CPD-341 (14.9 mg, 43% yield) as a yellow solid. MS (ESI) m/z=890.7 [M+H]⁺.

### Example 570. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(6-methoxypyridazin-3-yl)-4-(methylamino)benzamide (CPD-343)

### Scheme 570

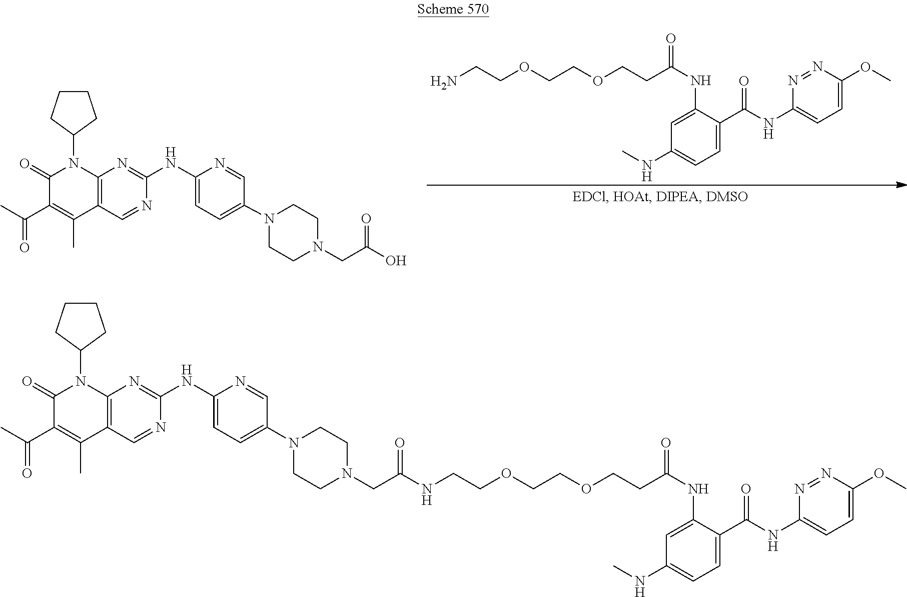 [SMILES: CC(=O)c1c(C)c2cnc(Nc3ccc(N4CCN(CC(=O)O)CC4)cn3)nc2n(C2CCCC2)c1=O ; CNc1ccc(C(=O)Nc2ccc(OC)nn2)c(NC(=O)CCOCCOCCN)c1 ; CNc1ccc(C(=O)Nc2ccc(OC)nn2)c(NC(=O)CCOCCOCCNC(=O)CN2CCN(c3ccc(Nc4ncc5c(C)c(C(C)=O)c(=O)n(C6CCCC6)c5n4)nc3)CC2)c1]

CPD-343 was synthesized following the standard procedure for preparing CPD-341 (6.6 mg, 19% yield) as a yellow solid. MS (ESI) m/z=920.5 [M+H]⁺.

### Example 571. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(6-methoxypyridazin-3-yl)-4-(methylamino)benzamide (CPD-344)

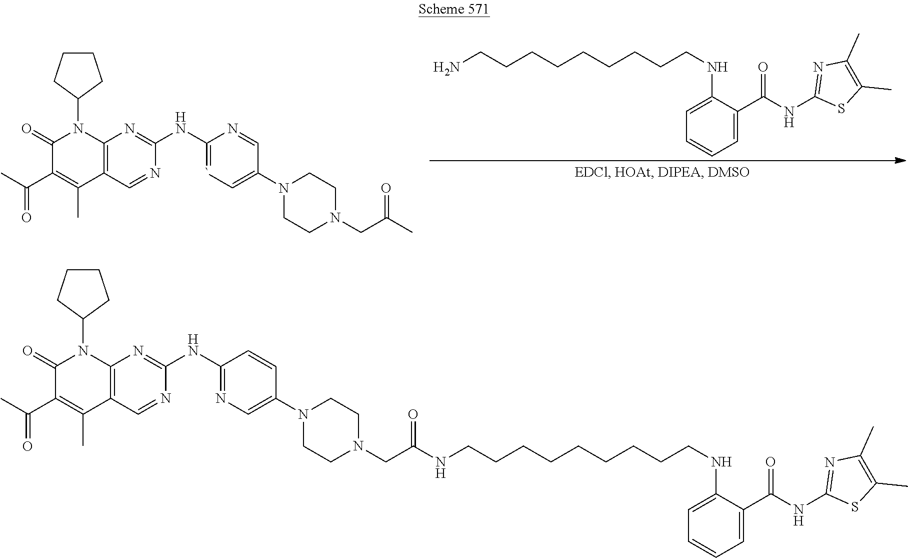

CPD-344 was synthesized following the standard procedure for preparing CPD-341 (1.4 mg, 2% yield) as a yellow solid. MS (ESI) m/z=876.9 [M+H]⁺.

### Example 572. 2-(3-(4-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)piperazin-1-yl)propanamido)-N-(6-methoxypyridazin-3-yl)benzamide (CPD-345)

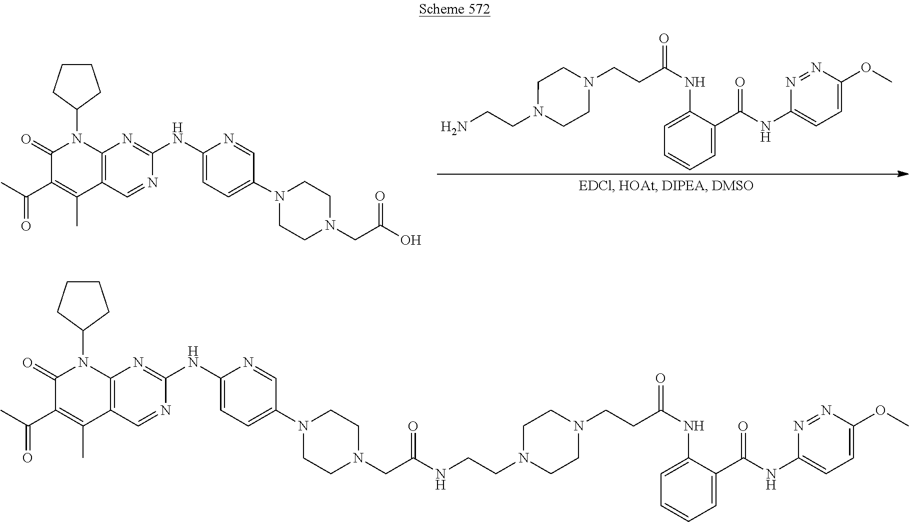 [SMILES: CC(=O)c1c(C)c2cnc(Nc3ccc(N4CCN(CC(=O)O)CC4)cn3)nc2n(C2CCCC2)c1=O ; COc1ccc(NC(=O)c2ccccc2NC(=O)CCN2CCN(CCN)CC2)nn1 ; COc1ccc(NC(=O)c2ccccc2NC(=O)CCN2CCN(CCNC(=O)CN3CCN(c4ccc(Nc5ncc6c(C)c(C(C)=O)c(=O)n(C7CCCC7)c6n5)nc4)CC3)CC2)nn1]

CPD-345 was synthesized following the standard procedure for preparing CPD-341 (2.5 mg, 10% yield) as a yellow solid. MS (ESI) m/z=915.8 [M+H]⁺.

### Example 573. 2-(5-(4-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)piperidin-1-yl)pentanamido)-N-(6-methoxypyridazin-3-yl)benzamide (CPD-346)

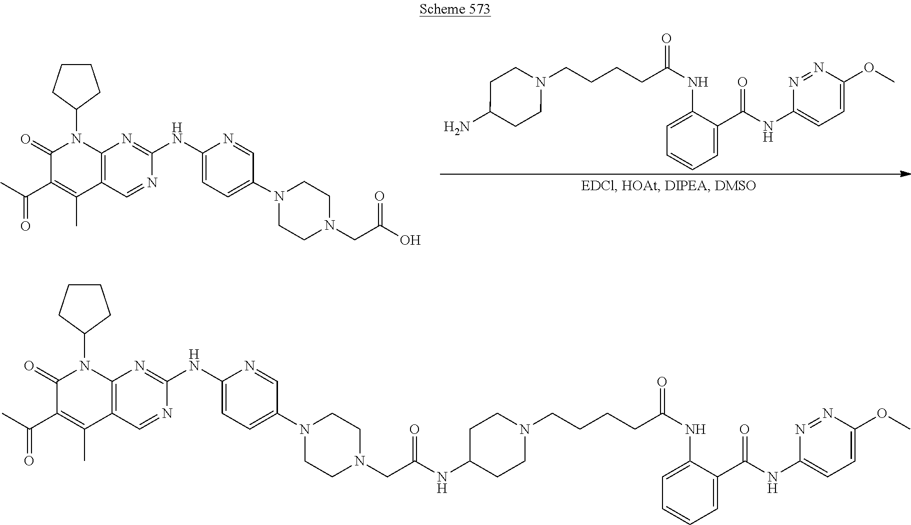 [SMILES: CC(=O)c1c(C)c2cnc(Nc3ccc(N4CCN(CC(=O)O)CC4)cn3)nc2n(C2CCCC2)c1=O ; COc1ccc(NC(=O)c2ccccc2NC(=O)CCCCN2CCC(N)CC2)nn1 ; COc1ccc(NC(=O)c2ccccc2NC(=O)CCCCN2CCC(NC(=O)CN3CCN(c4ccc(Nc5ncc6c(C)c(C(C)=O)c(=O)n(C7CCCC7)c6n5)nc4)CC3)CC2)nn1]

CPD-346 was synthesized following the standard procedure for preparing CPD-341 (14.8 mg, 78% yield) as a yellow solid. MS (ESI) m/z=914.8 [M+H]⁺.

### Example 574. 2-(2-(2-(4-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)piperidin-1-yl)ethoxy)acetamido)-N-(6-methoxypyridazin-3-yl)benzamide (CPD-347)

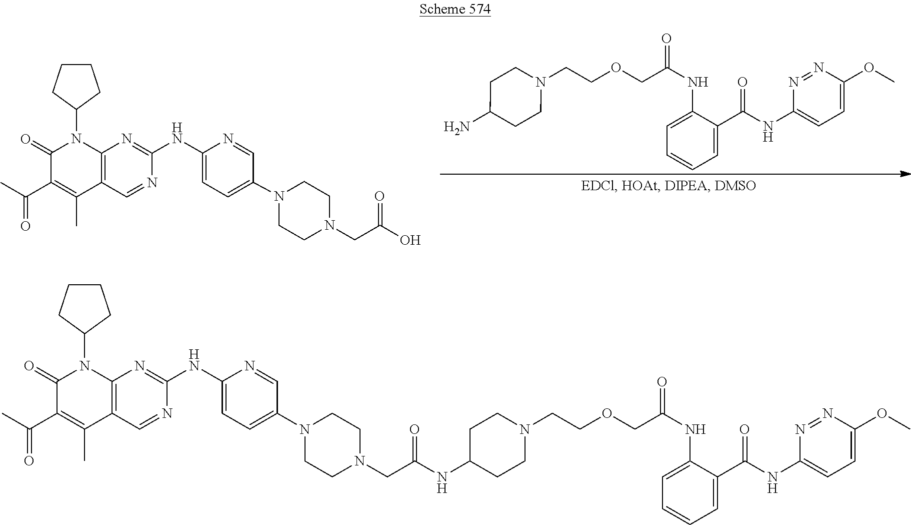 [SMILES: CC(=O)c1c(C)c2cnc(Nc3ccc(N4CCN(CC(=O)O)CC4)cn3)nc2n(C2CCCC2)c1=O ; COc1ccc(NC(=O)c2ccccc2NC(=O)COCCN2CCC(N)CC2)nn1 ; COc1ccc(NC(=O)c2ccccc2NC(=O)COCCN2CCC(NC(=O)CN3CCN(c4ccc(Nc5ncc6c(C)c(C(C)=O)c(=O)n(C7CCCC7)c6n5)nc4)CC3)CC2)nn1]

CPD-347 was synthesized following the standard procedure for preparing CPD-341 (20.5 mg, 90% yield) as a yellow solid. MS (ESI) m/z=916.6 [M+H]⁺.

### Example 575. 2-(3-(2-(4-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)piperidin-1-yl)ethoxy)propanamido)-N-(6-methoxypyridazin-3-yl)benzamide (CPD-348)

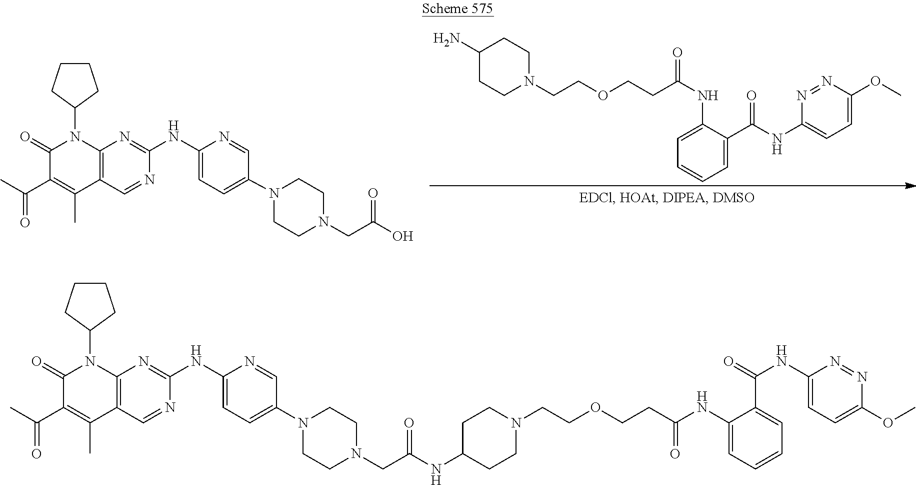 [SMILES: CC(=O)c1c(C)c2cnc(Nc3ccc(N4CCN(CC(=O)O)CC4)cn3)nc2n(C2CCCC2)c1=O ; COc1ccc(NC(=O)c2ccccc2NC(=O)CCOCCN2CCC(N)CC2)nn1 ; COc1ccc(NC(=O)c2ccccc2NC(=O)CCOCCN2CCC(NC(=O)CN3CCN(c4ccc(Nc5ncc6c(C)c(C(C)=O)c(=O)n(C7CCCC7)c6n5)nc4)CC3)CC2)nn1]

CPD-348 was synthesized following the standard procedure for preparing CPD-341 (5.7 mg, 34% yield) as a yellow solid. MS (ESI) m/z=930.7 [M+H]⁺.

### Example 576. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(6-cyclopropoxypyridazin-3-yl)benzamide (CPD-350)

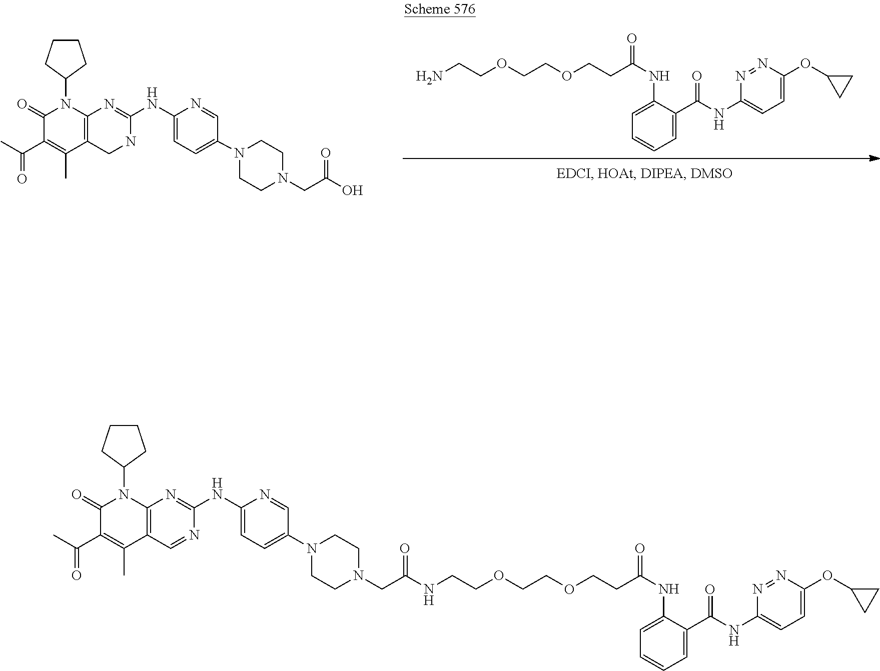 [SMILES: CC(=O)c1c(C)c2c(n(C3CCCC3)c1=O)N=C(Nc1ccc(N3CCN(CC(=O)O)CC3)cn1)NC2 ; NCCOCCOCCC(=O)Nc1ccccc1C(=O)Nc1ccc(OC2CC2)nn1 ; CC(=O)c1c(C)c2cnc(Nc3ccc(N4CCN(CC(=O)NCCOCCOCCC(=O)Nc5ccccc5C(=O)Nc5ccc(OC6CC6)nn5)CC4)cn3)nc2n(C2CCCC2)c1=O]

CPD-350 was synthesized following the standard procedure for preparing CPD-341 (4.8 mg, 10% yield) as a yellow solid. MS (ESI) m/z=917.7 [M+H]⁺.

### Example 577. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(6-isopropoxypyridazin-3-yl)benzamide (CPD-351)

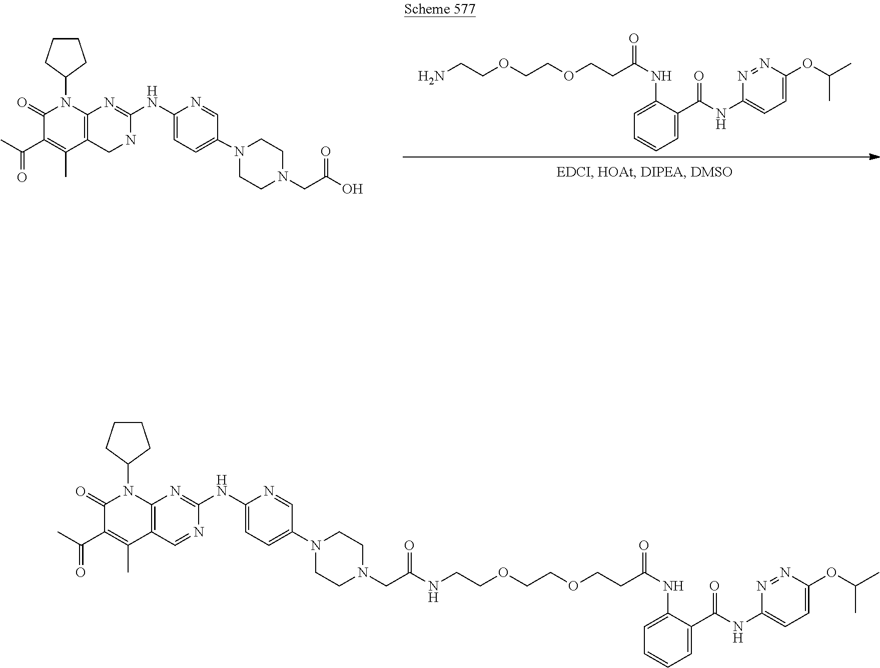 [SMILES: CC(=O)c1c(C)c2c(n(C3CCCC3)c1=O)N=C(Nc1ccc(N3CCN(CC(=O)O)CC3)cn1)NC2 ; CC(C)Oc1ccc(NC(=O)c2ccccc2NC(=O)CCOCCOCCN)nn1 ; CC(=O)c1c(C)c2cnc(Nc3ccc(N4CCN(CC(=O)NCCOCCOCCC(=O)Nc5ccccc5C(=O)Nc5ccc(OC(C)C)nn5)CC4)cn3)nc2n(C2CCCC2)c1=O]

CPD-351 was synthesized following the standard procedure for preparing CPD-341 (10.1 mg, 21% yield) as a yellow solid. MS (ESI) m/z=919.6 [M+H]⁺.

### Example 578. (S)-2-(10-((2-(2-(4-(4-chlorophenyl)-2,3,9-trimethyl-6H-thieno[3,2-f][1,2,4]triazolo[4,3-a][1,4]diazepin-6-yl)acetamido)ethyl)amino) decanamido)-N-(4,5-dimethyl-thiazol-2-yl)benzamide (CPD-353)

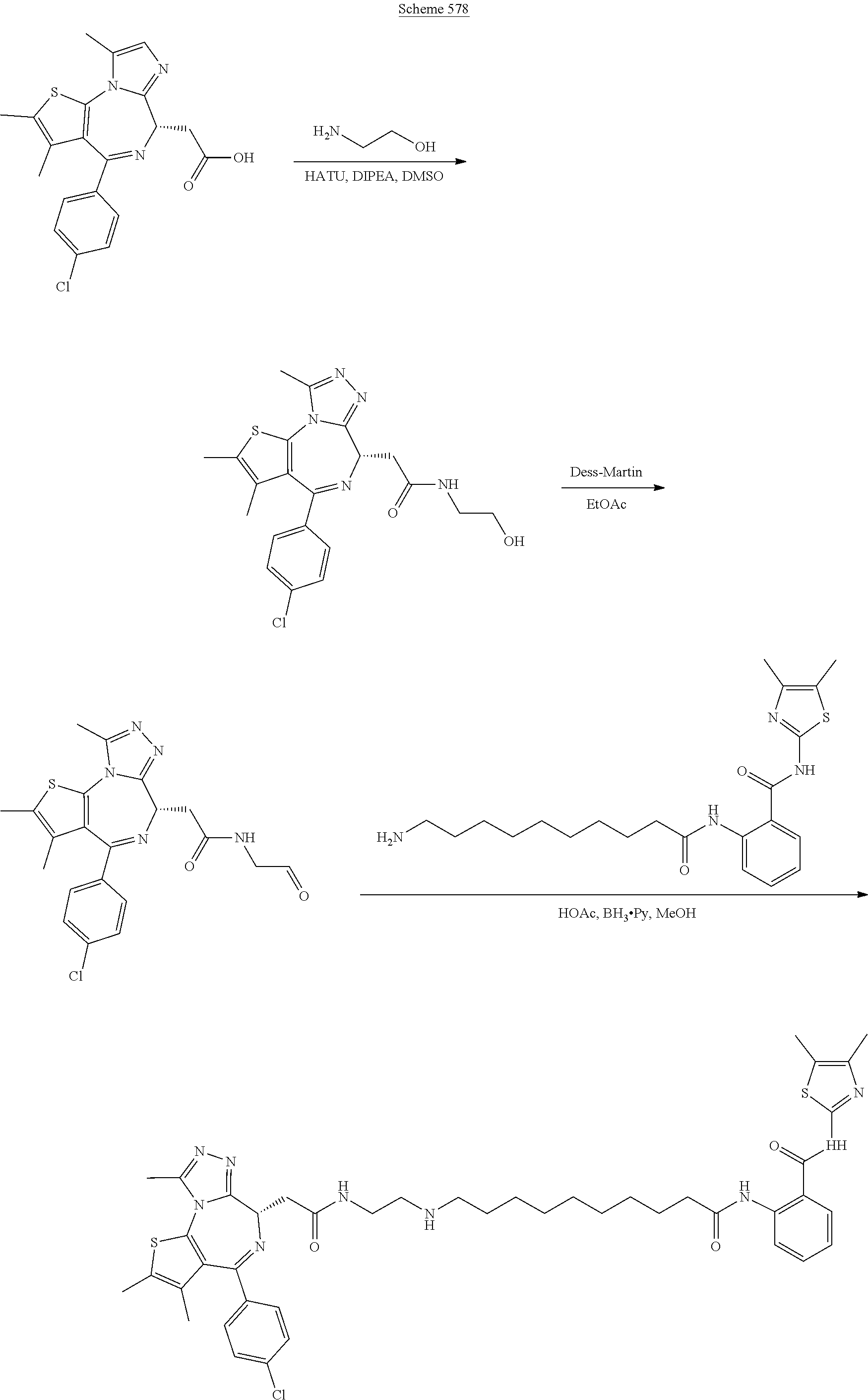

### Step 1. Synthesis of (S)-2-(4-(4-chlorophenyl)-2,3,9-trimethyl-6H-thieno[3,2-f][1,2,4]triazolo[4,3-a][1,4]diazepin-6-yl)-N-(2-hydroxyethyl)acetamide

To a solution of (S)-2-(4-(4-chlorophenyl)-2,3,9-trimethyl-6H-thieno[3,2-f][1,2,4]triazolo[4,3-a][1,4]diazepin-6-yl)acetic acid (234 mg, 0.58 mmol) and 2-aminoethanol (HCl salt, 74.02 mg, 0.76 mmol) in DMSO (3 mL) was added HATU (332.9 mg, 0.88 mmol) and DIPEA (301.7 mg, 2.33 mmol). The mixture was stirred at rt for 0.5 h, before it was purified by reverse phase chromatography to provide the title compound (248 mg, 94% yield) as a white solid. MS (ESI) m/z=444.15 [M+H]⁺.

### Step 2. Synthesis of (S)-2-(4-(4-chlorophenyl)-2,3,9-trimethyl-6H-thieno[3,2-f][1,2,4]triazolo[4,3-a][1,4]diazepin-6-yl)-N-(2-oxoethyl) acetamide

To a solution of (S)-2-(4-(4-chlorophenyl)-2,3,9-trimethyl-6H-thieno[3,2-f][1,2,4]triazolo[4,3-a][1,4]diazepin-6-yl)-N-(2-hydroxyethyl)acetamide (100 mg, 0.23 mmol) in EtOAc (3 mL) was added Dess-Martin periodinane (143.31 mg, 0.34 mmol). The mixture was stirred at 30° C. for 6 h, before it was filtered and concentrated under reduced pressure. The residue was purified by reverse phase chromatography to provide the title compound (85 mg, 84% yield) as a white solid. MS (ESI) m/z=442.11 [M+H]⁺.

### Step 3. Synthesis of (S)-2-(10-((2-(2-(4-(4-chlorophenyl)-2,3,9-trimethyl-6H-thieno[3,2-f][1,2,4]triazolo[4,3-a][1,4]diazepin-6-yl)acetamido)ethyl)amino) decanamido)-N-(4,5-dimethyl-thiazol-2-yl)benzamide

To a solution of HOAc (15 mmol) in methanol (3 mL) were added (S)-2-(4-(4-chlorophenyl)-2,3,9-trimethyl-6H-thieno[3,2-f][1,2,4]triazolo[4,3-a][1,4]diazepin-6-yl)-N-(2-oxo-ethyl)acetamide (22 mg, 0.05 mmol) and 2-(10-aminodecanoylamino)-N-(4,5-dimethylthiazol-2-yl)benzamide (20.74 mg, 0.05 mmol). The mixture was stirred at rt for 0.5 h, before borane-2-picoline complex (10.65 mg, 0.1 mmol) was added. The mixture was stirred at rt for another 12 h, before it was concentrated under reduced pressure. The residue was purified by reverse phase chromatography followed by prep-HPLC to provide the title compound (8 mg, 19% yield) as a white solid. MS (ESI) m/z=422.02 [M/2+H]⁺.

### Example 579. (S)-2-(8-((2-(2-(4-(4-chlorophenyl)-2,3,9-trimethyl-6H-thieno[3,2-f][1,2,4]triazolo[4,3-a][1,4]diazepin-6-yl)acetamido)ethyl)amino)octanamido)-N-(4,5-dimethylthiazol-2-yl)benzamide (CPD-354)

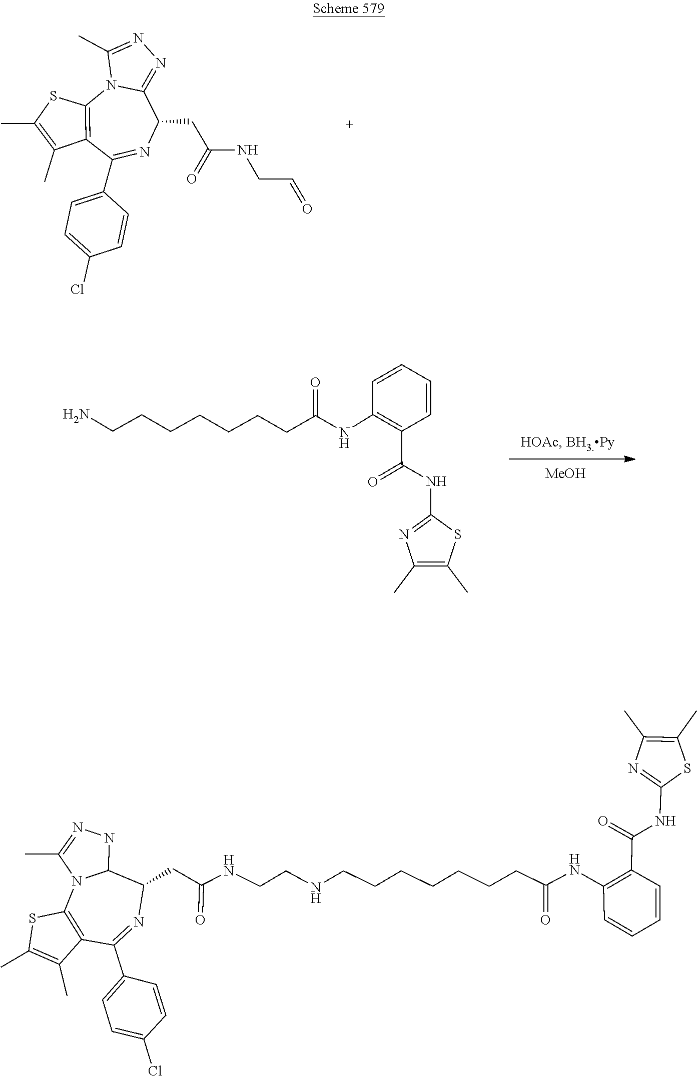

CPD-354 was synthesized following the standard procedure for step 3 of the preparation of CPD-353 (1.93 mg, 10% yield) as a white solid. MS (ESI) m/z=408.4 [M/2+H]⁺.

### Example 580. (S)-2-(6-((2-(2-(4-(4-chlorophenyl)-2,3,9-trimethyl-6H-thieno[3,2-f][1,2,4]triazolo[4,3-a][1,4]diazepin-6-yl)acetamido)ethyl)amino)hexanamido)-N-(4,5-dimethylthiazol-2-yl)benzamide (CPD-355)

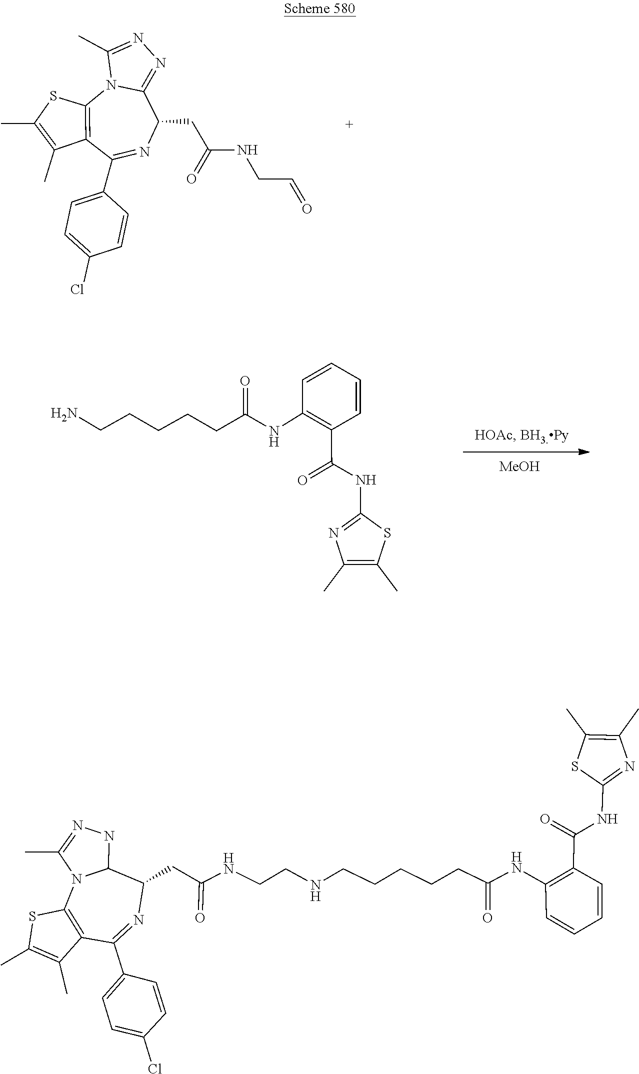

CPD-355 was synthesized following the standard procedure for step 3 of the preparation of CPD-353 (2.29 mg, 26% yield) as a white solid. MS (ESI) m/z=394.4 [M/2+H]⁺.

### Example 581. (S)-2-(4-((2-(2-(4-(4-chlorophenyl)-2,3,9-trimethyl-6H-thieno[3,2-f][1,2,4]triazolo[4,3-a][1,4]diazepin-6-yl)acetamido)ethyl)amino)butanamido)-N-(4,5-dimethylthiazol-2-yl)benzamide (CPD-356)

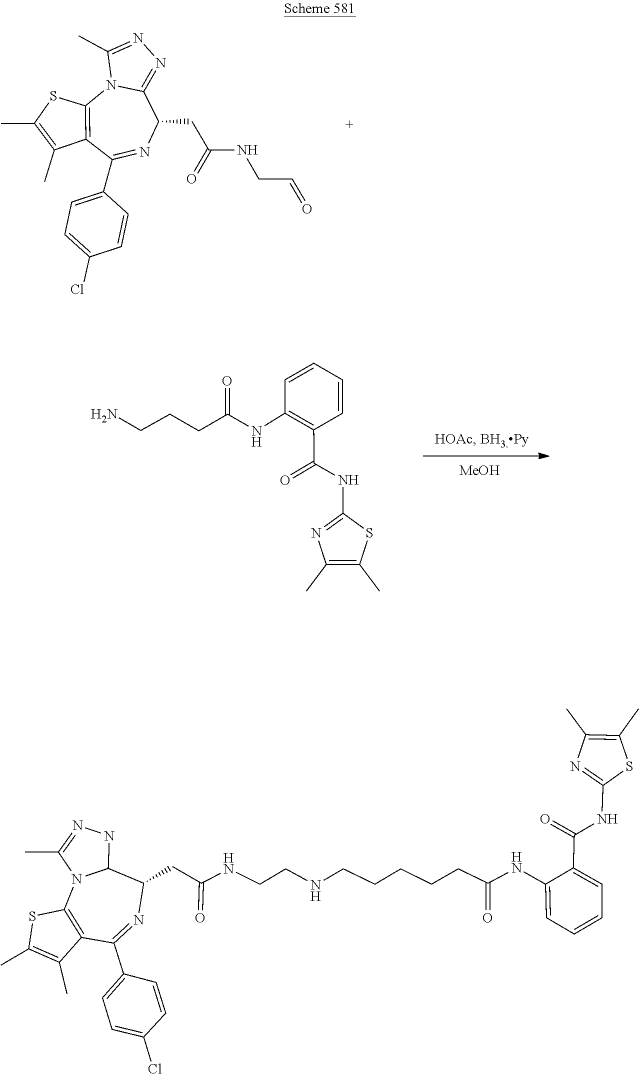

CPD-356 was synthesized following the standard procedure for step 3 of the preparation of CPD-353 (7.8 mg, 45% yield) as a white solid. MS (ESI) m/z=380.01 [M/2+H]⁺.

### Example 582. 2-(3-(2-(2-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-3-oxopropoxy)ethoxy)ethoxy)propanamido)-N-(4,5-dimethylthiazol-2-yl)benzamide (CPD-357)

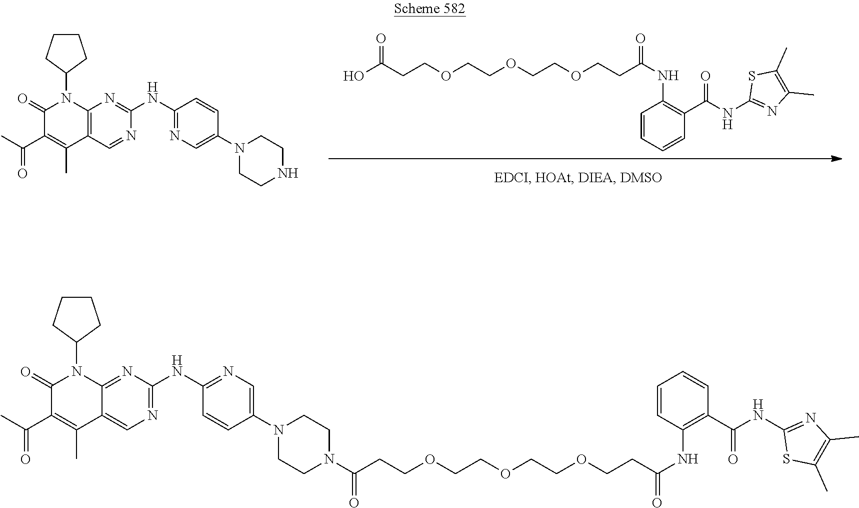 [SMILES: CC(=O)c1c(C)c2cnc(Nc3ccc(N4CCNCC4)cn3)nc2n(C2CCCC2)c1=O ; Cc1nc(NC(=O)c2ccccc2NC(=O)CCOCCOCCOCCC(=O)O)sc1C ; CC(=O)c1c(C)c2cnc(Nc3ccc(N4CCN(C(=O)CCOCCOCCOCCC(=O)Nc5ccccc5C(=O)Nc5nc(C)c(C)s5)CC4)cn3)nc2n(C2CCCC2)c1=O]

CPD-357 was synthesized following the standard procedure for preparing CPD-341 (2.3 mg, 11% yield) as a yellow solid. MS (ESI) m/z=909.7 [M+H]⁺.

### Example 583. 7-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methylpyridin-2-yl)-1,2,3,4-tetrahydroquinoline-6-carboxamide (CPD-358)

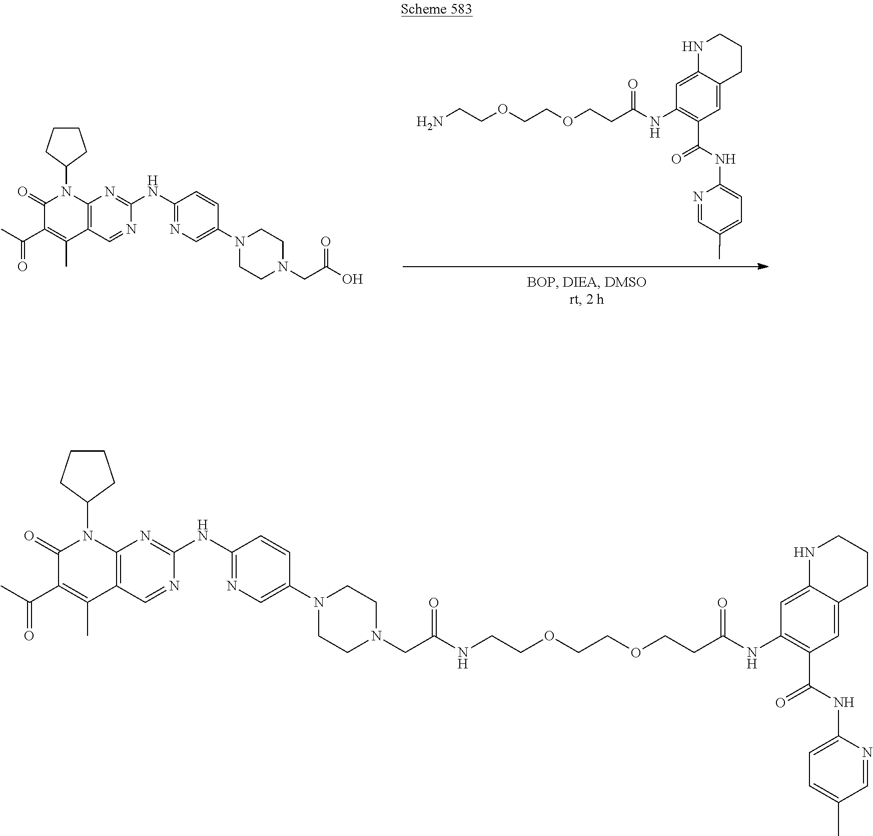 [SMILES: CC(=O)c1c(C)c2cnc(Nc3ccc(N4CCN(CC(=O)O)CC4)cn3)nc2n(C2CCCC2)c1=O ; Cc1ccc(NC(=O)c2cc3c(cc2NC(=O)CCOCCOCCN)NCCC3)nc1 ; CC(=O)c1c(C)c2cnc(Nc3ccc(N4CCN(CC(=O)NCCOCCOCCC(=O)Nc5cc6c(cc5C(=O)Nc5ccc(C)cn5)CCCN6)CC4)cn3)nc2n(C2CCCC2)c1=O]

CPD-358 was synthesized following the standard procedure for preparing CPD-336 (8.8 mg, 42% yield) as a yellow solid. MS (ESI) m/z=929.48 [M+H]⁺.

### Example 584. 2-(3-(2-(2-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-3-oxopropoxy)ethoxy)ethoxy)propanamido)-N-(4,5-dimethylthiazol-2-yl)benzamide (CPD-359)

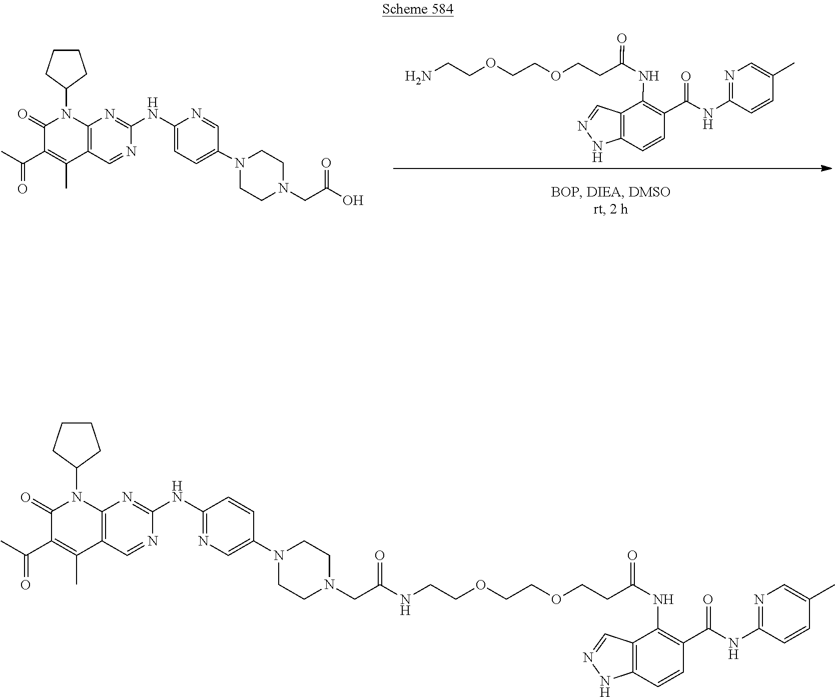 [SMILES: CC(=O)c1c(C)c2cnc(Nc3ccc(N4CCN(CC(=O)O)CC4)cn3)nc2n(C2CCCC2)c1=O ; Cc1ccc(NC(=O)c2ccc3[nH]ncc3c2NC(=O)CCOCCOCCN)nc1 ; CC(=O)c1c(C)c2cnc(Nc3ccc(N4CCN(CC(=O)NCCOCCOCCC(=O)Nc5c(C(=O)Nc6ccc(C)cn6)ccc6[nH]ncc56)CC4)cn3)nc2n(C2CCCC2)c1=O]

CPD-359 was synthesized following the standard procedure for preparing CPD-336 (4.7 mg, 28% yield) as a yellow solid. MS (ESI) m/z=914.5 [M+H]⁺.

### Example 585. 6-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)-ethoxy)propanamido)-N-(5-methylpyridin-2-yl)indoline-5-carboxamide (CPD-360)

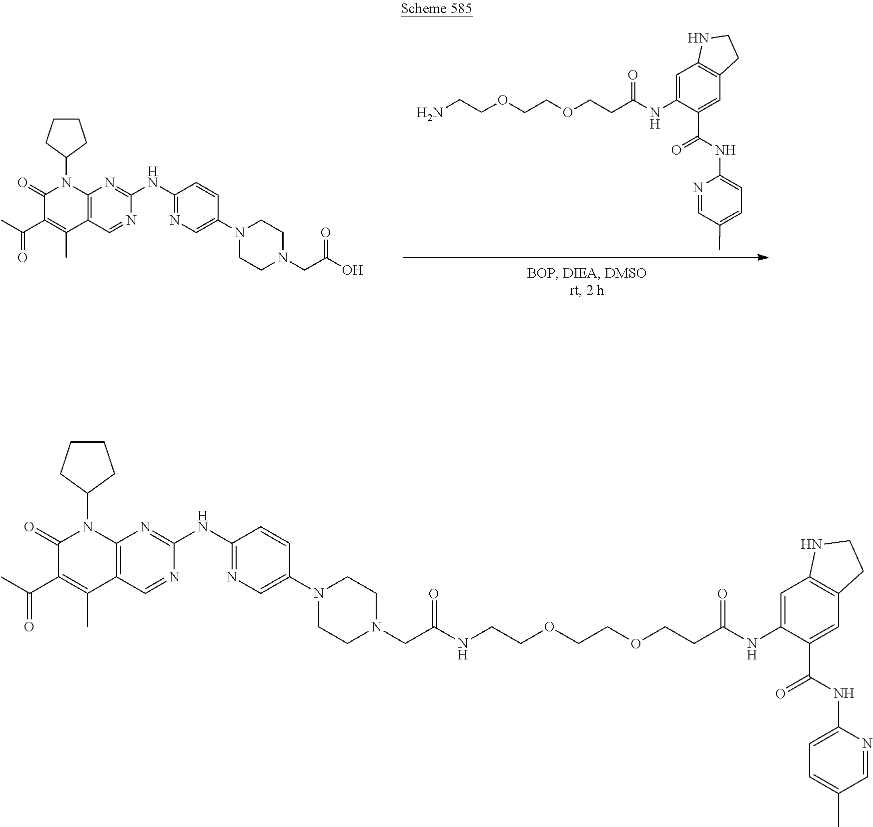 [SMILES: CC(=O)c1c(C)c2cnc(Nc3ccc(N4CCN(CC(=O)O)CC4)cn3)nc2n(C2CCCC2)c1=O ; Cc1ccc(NC(=O)c2cc3c(cc2NC(=O)CCOCCOCCN)NCC3)nc1 ; CC(=O)c1c(C)c2cnc(Nc3ccc(N4CCN(CC(=O)NCCOCCOCCC(=O)Nc5cc6c(cc5C(=O)Nc5ccc(C)cn5)CCN6)CC4)cn3)nc2n(C2CCCC2)c1=O]

CPD-360 was synthesized following the standard procedure for preparing CPD-336 (7.5 mg, 35% yield) as a yellow solid. MS (ESI) m/z=915.46 [M+H]⁺.

### Example 586. 4-(3-(2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methylpyridin-2-yl)-1H-indole-5-carboxamide (CPD-361)

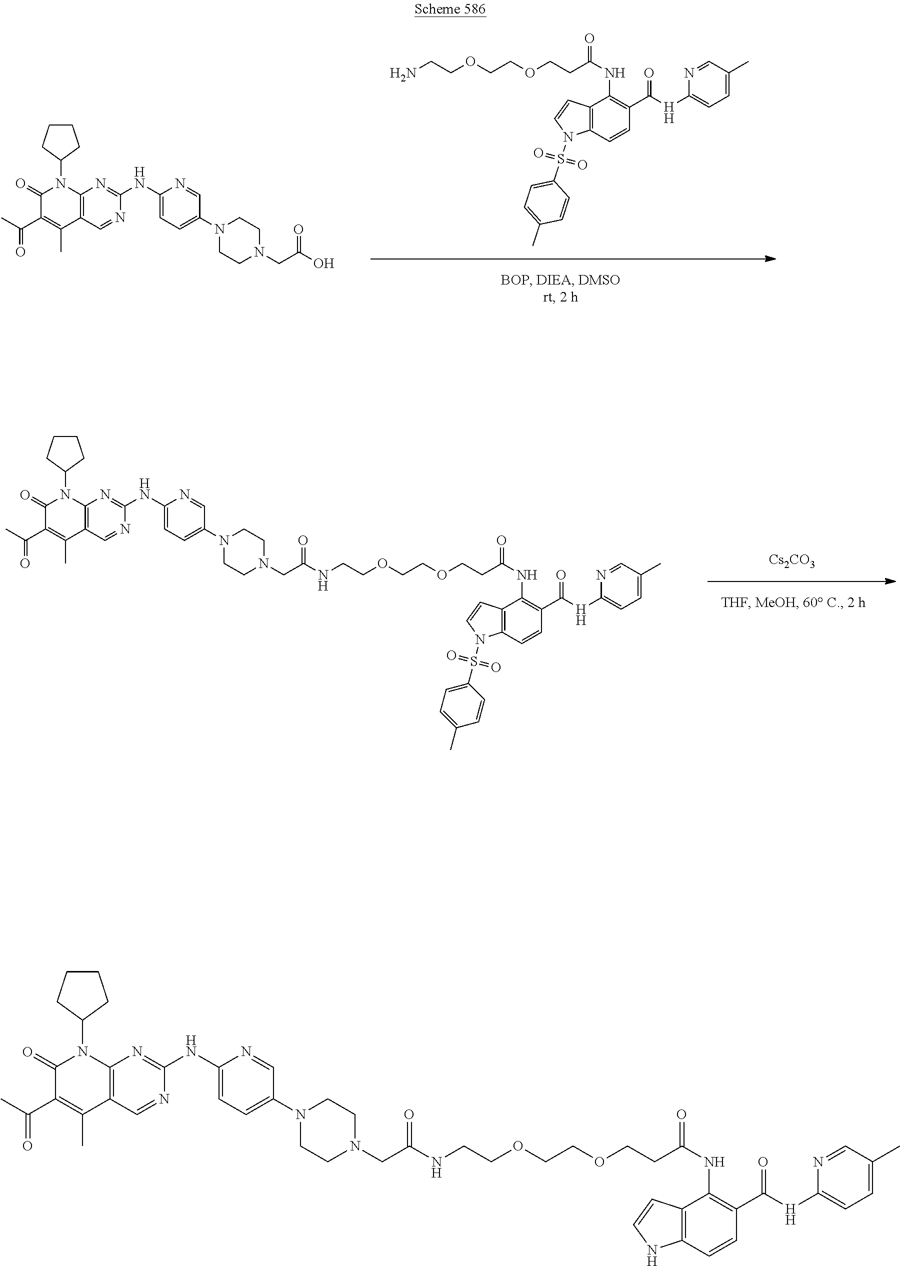

### Step 1. Synthesis of 4-(3-(2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methylpyridin-2-yl)-1-tosyl-1H-indole-5-carboxamide

The title compound was synthesized following the standard procedure for preparing CPD-336 (38 mg, 46% yield) as a yellow solid. MS (ESI) m/z=534.40 [M/2+H]⁺.

### Step 2. Synthesis of 4-(3-(2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methylpyridin-2-yl)-1H-indole-5-carboxamide

A mixture of 4-(3-(2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methylpyridin-2-yl)-1-tosyl-1H-indole-5-carboxamide (20 mg, 0.019 mmol) and Cs₂CO₃ (19.0 mg, 0.059 mmol) in THF (0.6 mL) was stirred at 60° C. for 2 h. The reaction mixture was combined with another batch of the reaction mixture starting from 4-(3-(2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methylpyridin-2-yl)-1-tosyl-1H-indole-5-carboxamide (14 mg, 0.013 mmol). The combined mixture was purified by prep-TLC (dichloromethane/methanol=15:1) followed by prep-HPLC to provide the title compound (10.6 mg, 36% yield) as a yellow solid. MS (ESI) m/z=457.4 [M/2+H]⁺.

### Example 587. 5-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methylpyridin-2-yl)quinoline-6-carboxamide (CPD-362)

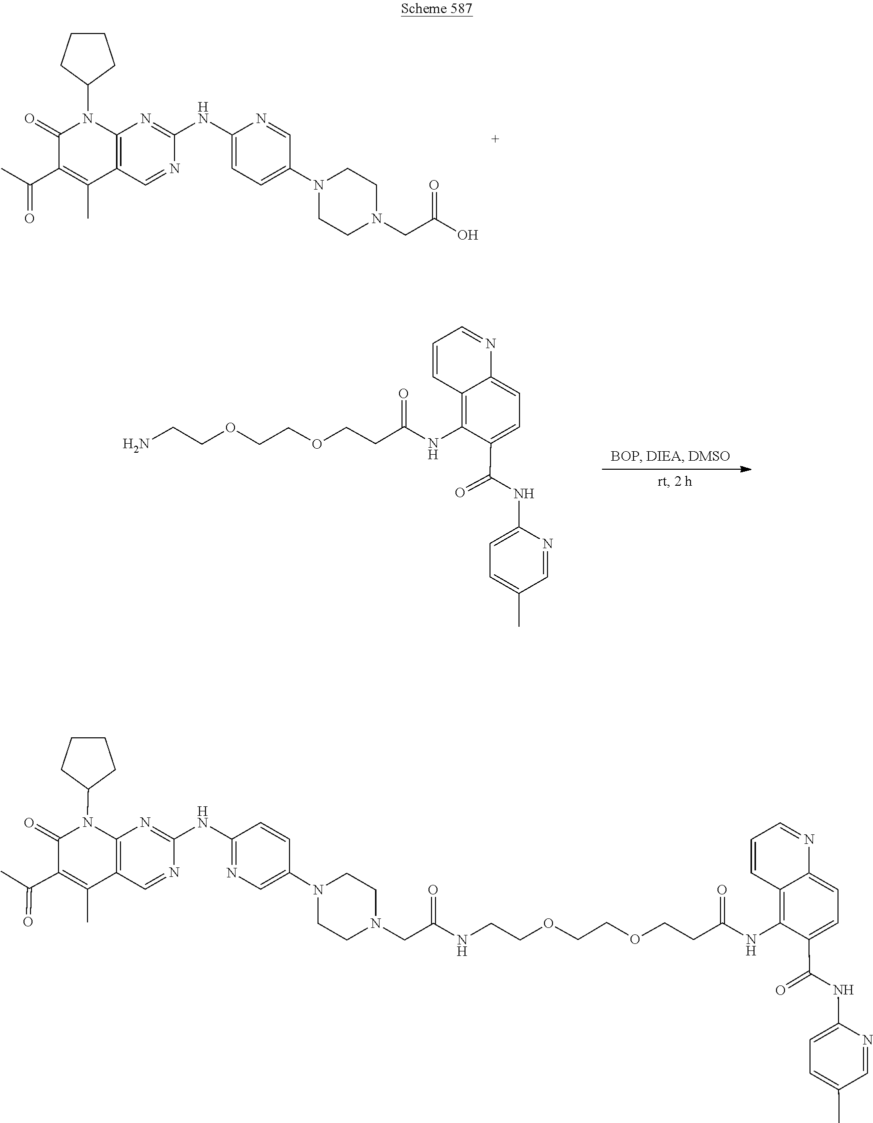 [SMILES: CC(=O)c1c(C)c2cnc(Nc3ccc(N4CCN(CC(=O)O)CC4)cn3)nc2n(C2CCCC2)c1=O ; "+" ; Cc1ccc(NC(=O)c2ccc3ncccc3c2NC(=O)CCOCCOCCN)nc1 ; CC(=O)c1c(C)c2cnc(Nc3ccc(N4CCN(CC(=O)NCCOCCOCCC(=O)Nc5c(C(=O)Nc6ccc(C)cn6)ccc6ncccc56)CC4)cn3)nc2n(C2CCCC2)c1=O]

CPD-362 was synthesized following the standard procedure for preparing CPD-336 (3.3 mg, 28% yield) as a yellow solid. MS (ESI) m/z=925.44 [M+H]⁺.

### Example 588. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(4,5-dimethylthiazol-2-yl)benzamide (CPD-363)

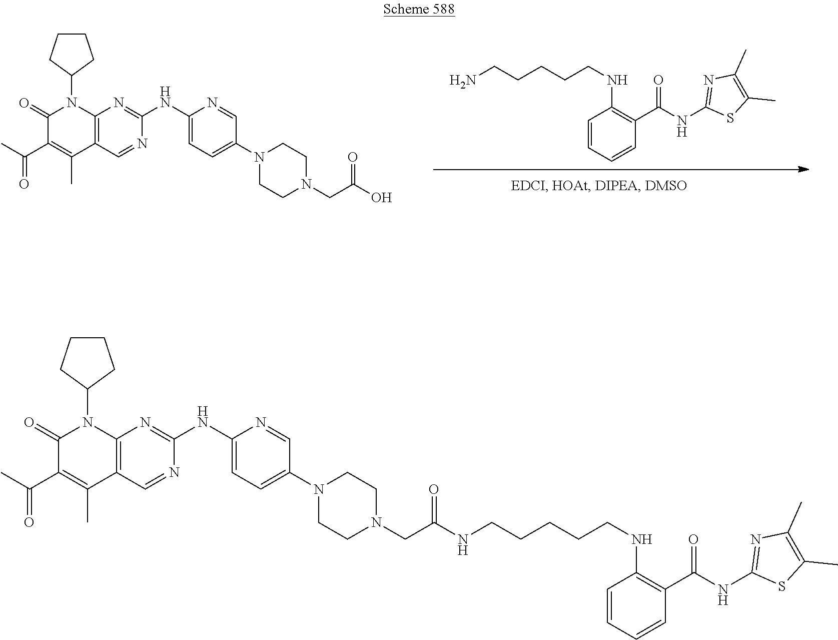 [SMILES: CC(=O)c1c(C)c2cnc(Nc3ccc(N4CCN(CC(=O)O)CC4)cn3)nc2n(C2CCCC2)c1=O ; Cc1nc(NC(=O)c2ccccc2NCCCCCN)sc1C ; CC(=O)c1c(C)c2cnc(Nc3ccc(N4CCN(CC(=O)NCCCCCNc5ccccc5C(=O)Nc5nc(C)c(C)s5)CC4)cn3)nc2n(C2CCCC2)c1=O]

CPD-363 was synthesized following the standard procedure for preparing CPD-341 (4.3 mg, 19% yield) as a yellow solid. MS (ESI) m/z=820.7 [M+H]⁺.

### Example 589. 2-((7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(4,5-dimethylthiazol-2-yl)benzamide (CPD-364)

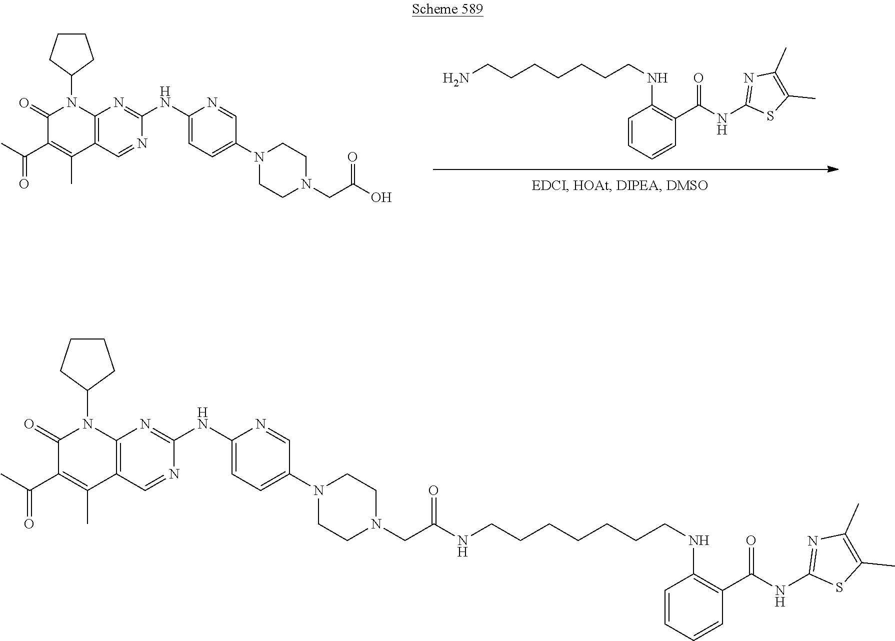 [SMILES: CC(=O)c1c(C)c2cnc(Nc3ccc(N4CCN(CC(=O)O)CC4)cn3)nc2n(C2CCCC2)c1=O ; Cc1nc(NC(=O)c2ccccc2NCCCCCCCN)sc1C ; CC(=O)c1c(C)c2cnc(Nc3ccc(N4CCN(CC(=O)NCCCCCCCNc5ccccc5C(=O)Nc5nc(C)c(C)s5)CC4)cn3)nc2n(C2CCCC2)c1=O]

CPD-364 was synthesized following the standard procedure for preparing CPD-341 (7.8 mg, 26% yield) as a yellow solid. MS (ESI) m/z=848.7 [M+H]⁺.

### Example 590. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-4-(dimethylamino)-N-(6-methoxypyridazin-3-yl)benzamide (CPD-365)

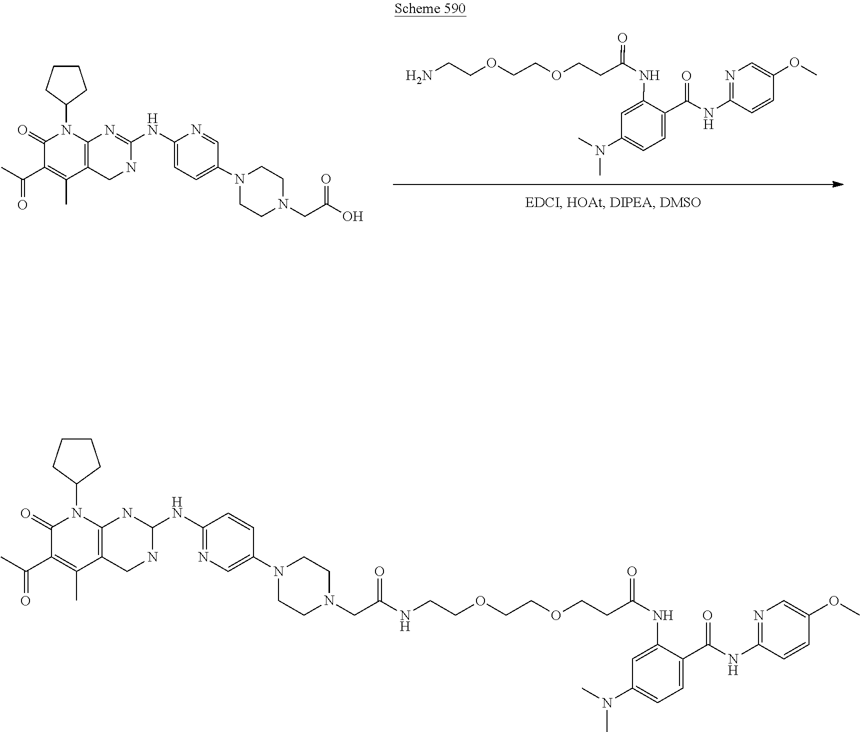 [SMILES: CC(=O)c1c(C)c2c(n(C3CCCC3)c1=O)N=C(Nc1ccc(N3CCN(CC(=O)O)CC3)cn1)NC2 ; COc1ccc(NC(=O)c2ccc(N(C)C)cc2NC(=O)CCOCCOCCN)nc1 ; COc1ccc(NC(=O)c2ccc(N(C)C)cc2NC(=O)CCOCCOCCNC(=O)CN2CCN(c3ccc(NC4NCc5c(C)c(C(C)=O)c(=O)n(C6CCCC6)c5N4)nc3)CC2)nc1]

CPD-365 was synthesized following the standard procedure for preparing CPD-341 (2.2 mg, 16% yield) as a yellow solid. MS (ESI) m/z=934.6 [M+H]⁺.

### Example 591. 2-(2-(2-((trans-4-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)oxy)ethoxy)acetamido)-N-(6-methoxypyridazin-3-yl)benzamide (CPD-366)

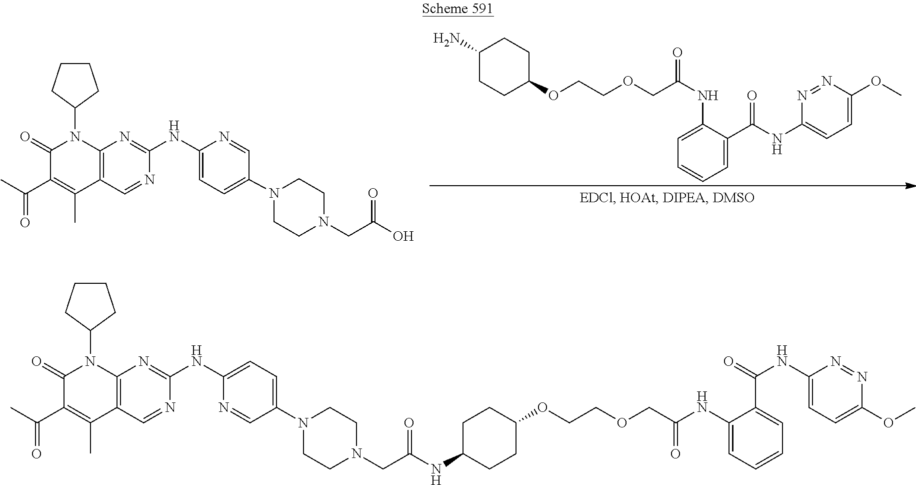 [SMILES: CC(=O)c1c(C)c2cnc(Nc3ccc(N4CCN(CC(=O)O)CC4)cn3)nc2n(C2CCCC2)c1=O ; COc1ccc(NC(=O)c2ccccc2NC(=O)COCCO[C@H]2CC[C@H](N)CC2)nn1 ; COc1ccc(NC(=O)c2ccccc2NC(=O)COCCO[C@H]2CC[C@H](NC(=O)CN3CCN(c4ccc(Nc5ncc6c(C)c(C(C)=O)c(=O)n(C7CCCC7)c6n5)nc4)CC3)CC2)nn1]

CPD-366 was synthesized following the standard procedure for preparing CPD-341 (15 mg, 40% yield) as a yellow solid. MS (ESI) m/z=931.6 [M+H]⁺.

### Example 592. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(5-methyl-1,3,4-thiadiazol-2-yl)benzamide (CPD-367)

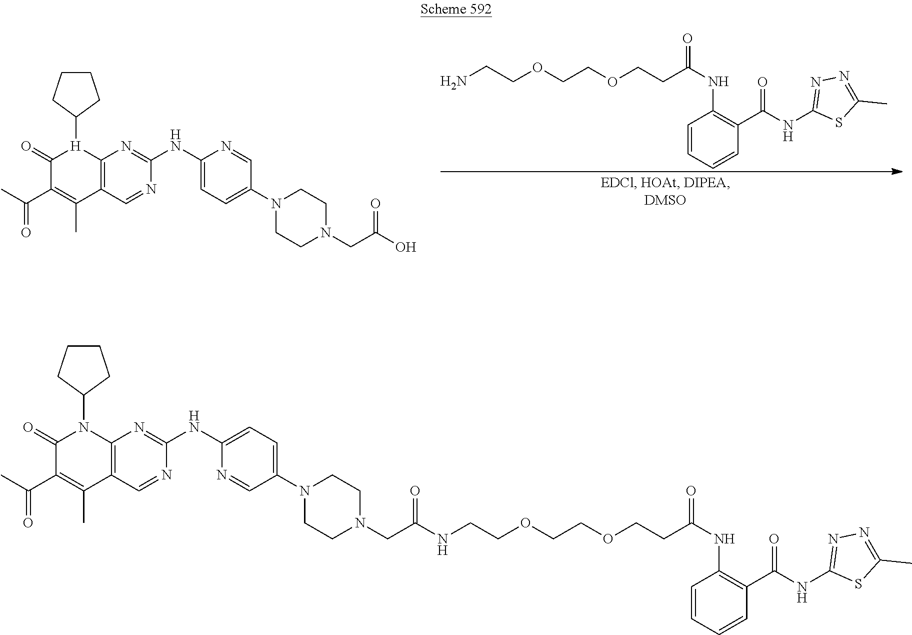

CPD-367 was synthesized following the standard procedure for preparing CPD-341 (15 mg, 40% yield) as a yellow solid. MS (ESI) m/z=881.5 [M+H]⁺.

### Example 593. 2-((7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(4,5-dimethylthiazol-2-yl)-4-methylbenzamide (CPD-368)

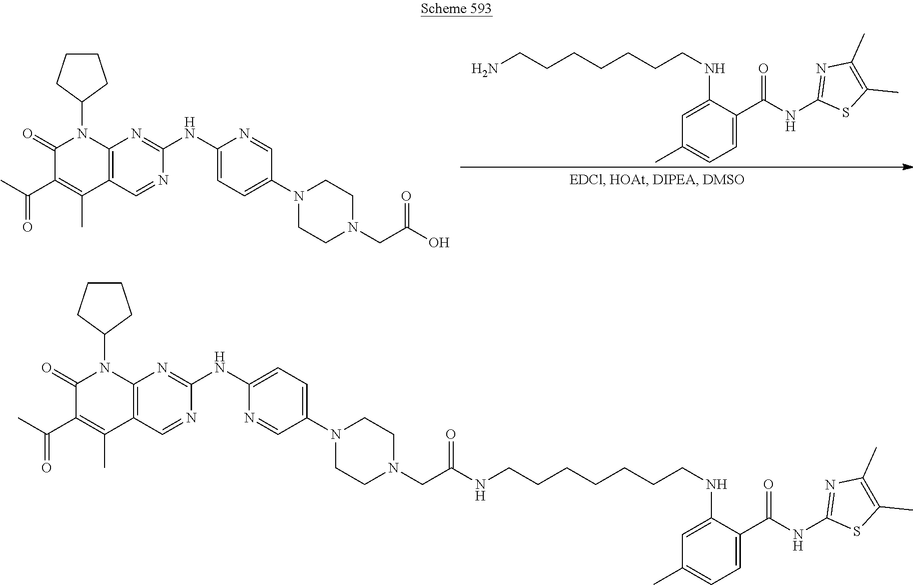 [SMILES: CC(=O)c1c(C)c2cnc(Nc3ccc(N4CCN(CC(=O)O)CC4)cn3)nc2n(C2CCCC2)c1=O ; Cc1ccc(C(=O)Nc2nc(C)c(C)s2)c(NCCCCCCCN)c1 ; CC(=O)c1c(C)c2cnc(Nc3ccc(N4CCN(CC(=O)NCCCCCCCNc5cc(C)ccc5C(=O)Nc5nc(C)c(C)s5)CC4)cn3)nc2n(C2CCCC2)c1=O]

CPD-368 was synthesized following the standard procedure for preparing CPD-341 (8 mg, 41% yield) as a yellow solid. MS (ESI) m/z=862.7 [M+H]⁺.

### Example 594. 2-(3-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(6-cyanopyridazin-3-yl)benzamide (CPD-369)

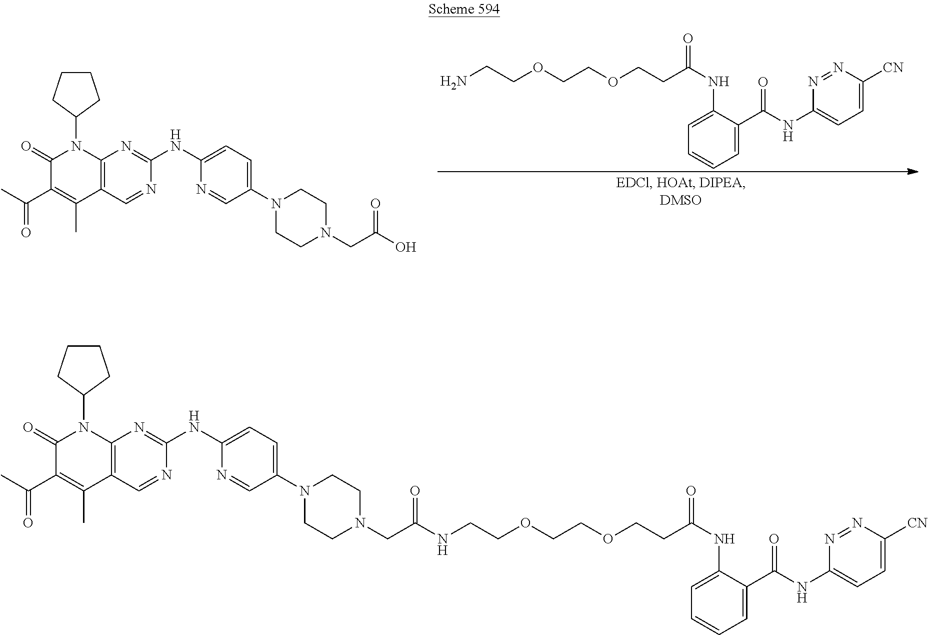 [SMILES: CC(=O)c1c(C)c2cnc(Nc3ccc(N4CCN(CC(=O)O)CC4)cn3)nc2n(C2CCCC2)c1=O ; N#Cc1ccc(NC(=O)c2ccccc2NC(=O)CCOCCOCCN)nn1 ; CC(=O)c1c(C)c2cnc(Nc3ccc(N4CCN(CC(=O)NCCOCCOCCC(=O)Nc5ccccc5C(=O)Nc5ccc(C#N)nn5)CC4)cn3)nc2n(C2CCCC2)c1=O]

CPD-369 was synthesized following the standard procedure for preparing CPD-341 (8.9 mg, 21% yield) as a yellow solid. MS (ESI) m/z=866.8 [M+H]⁺.

### Example 595. 2-((7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(4,5-dimethylthiazol-2-yl)-4-(methylamino)benzamide (CPD-370)

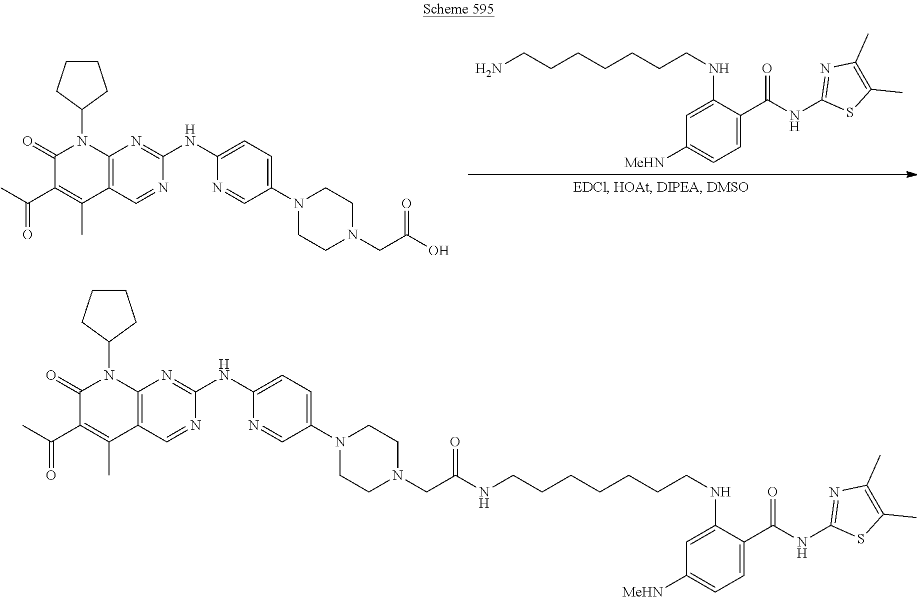 [SMILES: CC(=O)c1c(C)c2cnc(Nc3ccc(N4CCN(CC(=O)O)CC4)cn3)nc2n(C2CCCC2)c1=O ; CNc1ccc(C(=O)Nc2nc(C)c(C)s2)c(NCCCCCCCN)c1 ; CNc1ccc(C(=O)Nc2nc(C)c(C)s2)c(NCCCCCCCNC(=O)CN2CCN(c3ccc(Nc4ncc5c(C)c(C(C)=O)c(=O)n(C6CCCC6)c5n4)nc3)CC2)c1]

CPD-370 was synthesized following the standard procedure for preparing CPD-341 (4.4 mg, 18% yield) as a yellow solid. MS (ESI) m/z=877.5 [M+H]⁺.

### Example 596. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-cyclopropyl-5-methylpyridin-2-yl)-4-(methylamino)benzamide (CPD-371)

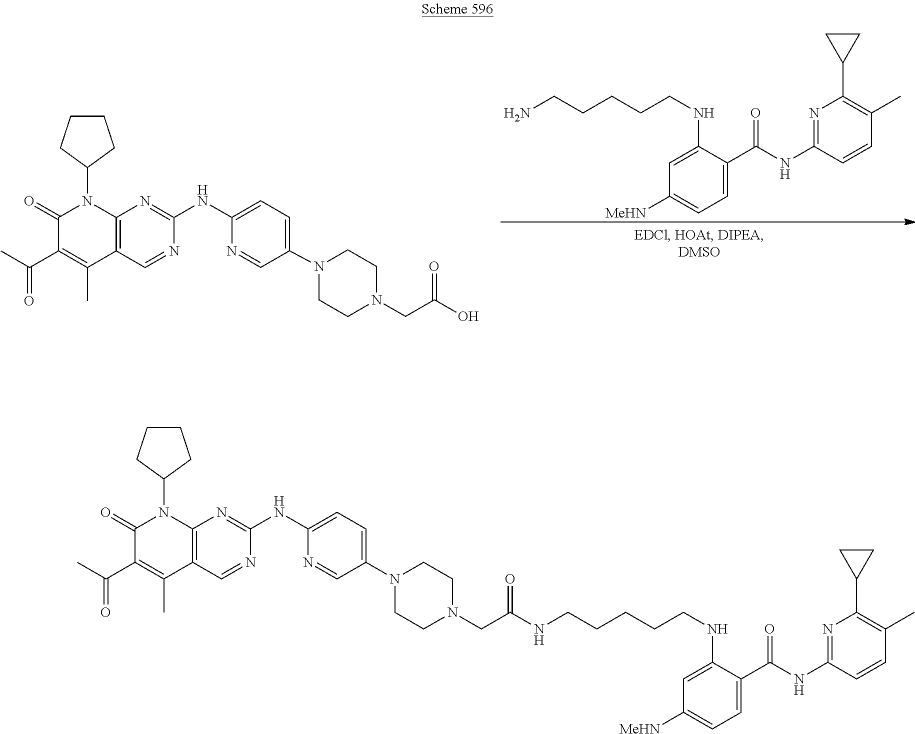 [SMILES: CC(=O)c1c(C)c2cnc(Nc3ccc(N4CCN(CC(=O)O)CC4)cn3)nc2n(C2CCCC2)c1=O ; CNc1ccc(C(=O)Nc2ccc(C)c(C3CC3)n2)c(NCCCCCN)c1 ; CNc1ccc(C(=O)Nc2ccc(C)c(C3CC3)n2)c(NCCCCCNC(=O)CN2CCN(c3ccc(Nc4ncc5c(C)c(C(C)=O)c(=O)n(C6CCCC6)c5n4)nc3)CC2)c1]

CPD-371 was synthesized following the standard procedure for preparing CPD-341 (4.3 mg, 27% yield) as a yellow solid. MS (ESI) m/z=869.8 [M+H]⁺.

### Example 597. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-4-fluorobenzamide (CPD-372)

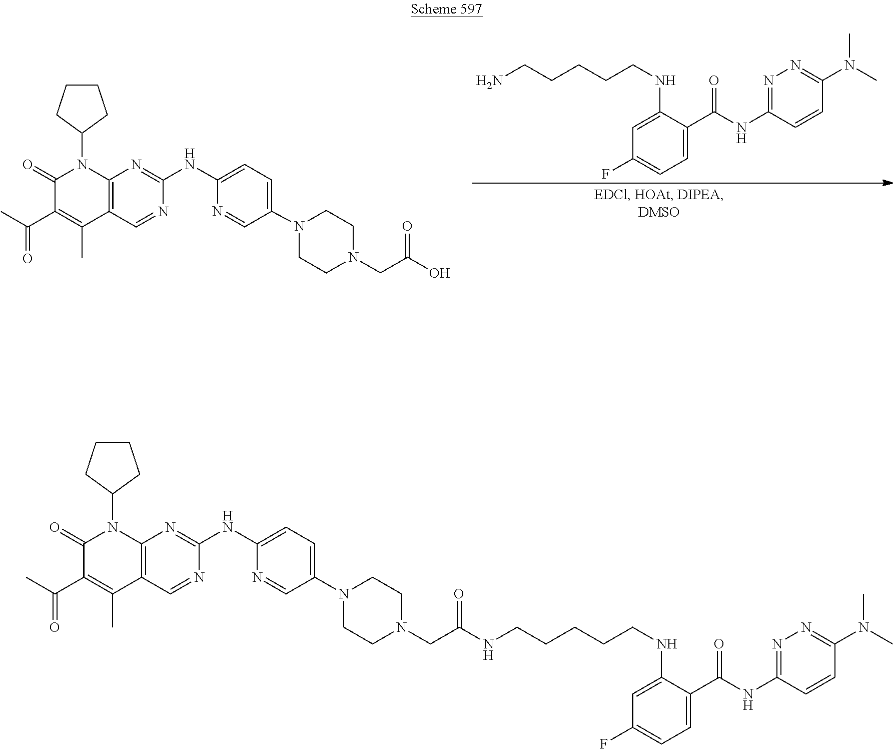 [SMILES: CC(=O)c1c(C)c2cnc(Nc3ccc(N4CCN(CC(=O)O)CC4)cn3)nc2n(C2CCCC2)c1=O ; CN(C)c1ccc(NC(=O)c2ccc(F)cc2NCCCCCN)nn1 ; CC(=O)c1c(C)c2cnc(Nc3ccc(N4CCN(CC(=O)NCCCCCNc5cc(F)ccc5C(=O)Nc5ccc(N(C)C)nn5)CC4)cn3)nc2n(C2CCCC2)c1=O]

CPD-372 was synthesized following the standard procedure for preparing CPD-341 (10.6 mg, 17% yield) as a yellow solid. MS (ESI) m/z=848.6 [M+H]⁺.

### Example 598. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-4-methylbenzamide (CPD-373)

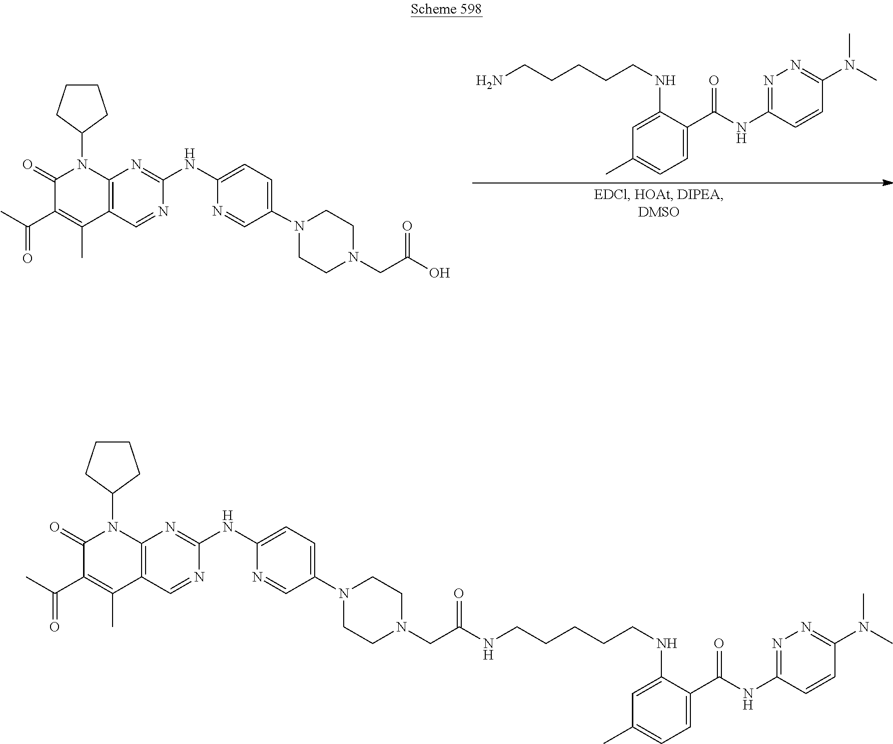 [SMILES: CC(=O)c1c(C)c2cnc(Nc3ccc(N4CCN(CC(=O)O)CC4)cn3)nc2n(C2CCCC2)c1=O ; Cc1ccc(C(=O)Nc2ccc(N(C)C)nn2)c(NCCCCCN)c1 ; CC(=O)c1c(C)c2cnc(Nc3ccc(N4CCN(CC(=O)NCCCCCNc5cc(C)ccc5C(=O)Nc5ccc(N(C)C)nn5)CC4)cn3)nc2n(C2CCCC2)c1=O]

CPD-373 was synthesized following the standard procedure for preparing CPD-341 (5 mg, 5% yield) as a yellow solid. MS (ESI) m/z=844.7 [M+H]⁺.

### Example 599. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-4-chloro-N-(6-(dimethylamino)pyridazin-3-yl)benzamide (CPD-374)

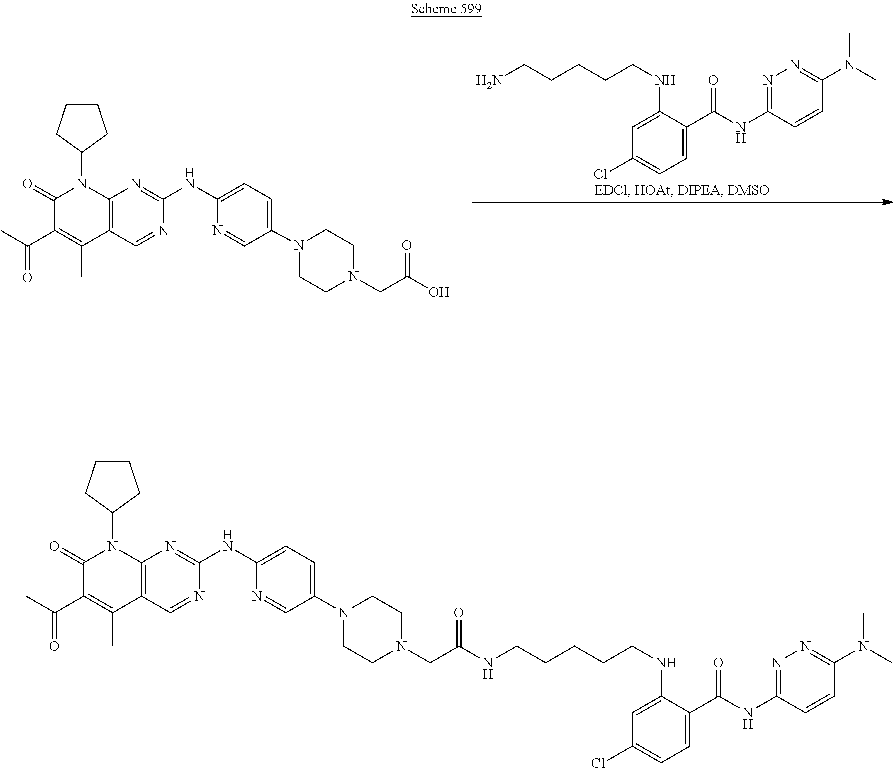 [SMILES: CC(=O)c1c(C)c2cnc(Nc3ccc(N4CCN(CC(=O)O)CC4)cn3)nc2n(C2CCCC2)c1=O ; CN(C)c1ccc(NC(=O)c2ccc(Cl)cc2NCCCCCN)nn1 ; CC(=O)c1c(C)c2cnc(Nc3ccc(N4CCN(CC(=O)NCCCCCNc5cc(Cl)ccc5C(=O)Nc5ccc(N(C)C)nn5)CC4)cn3)nc2n(C2CCCC2)c1=O]

CPD-374 was synthesized following the standard procedure for preparing CPD-341 (11.8 mg, 12% yield) as a yellow solid. MS (ESI) m/z=864.8 [M+H]⁺.

### Example 600. 2-((4-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)butyl)amino)-N-(6-(dimethylamino)pyridazin-3-yl)-4-methylbenzamide (CPD-375)

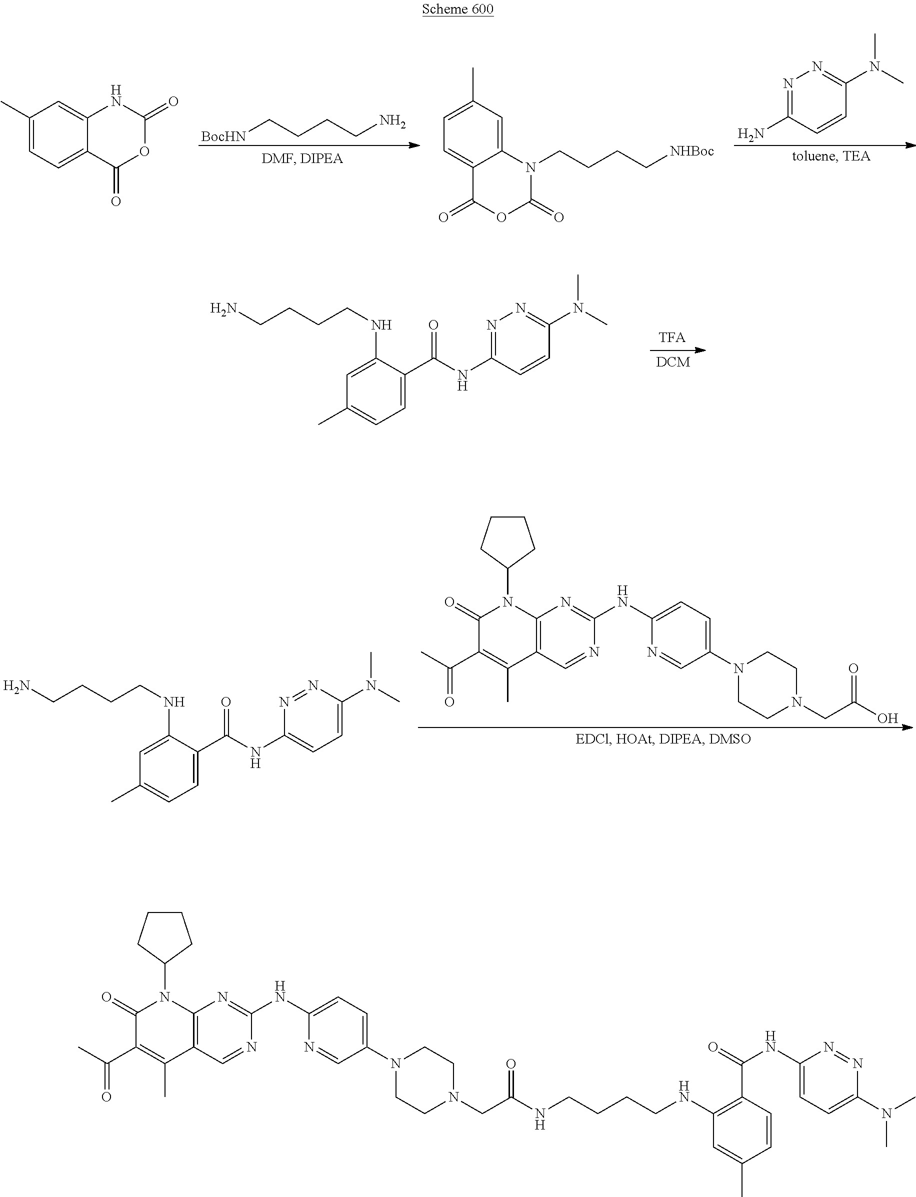

CPD-375 was synthesized following the standard procedure for preparing BL1-238 and CPD-341 (3.7 mg, 1% yield over 4 steps) as a yellow solid. MS (ESI) m/z=830.7 [M+H]⁺.

### Example 601. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-4-cyano-N-(6-(dimethylamino)pyridazin-3-yl)benzamide (CPD-376)

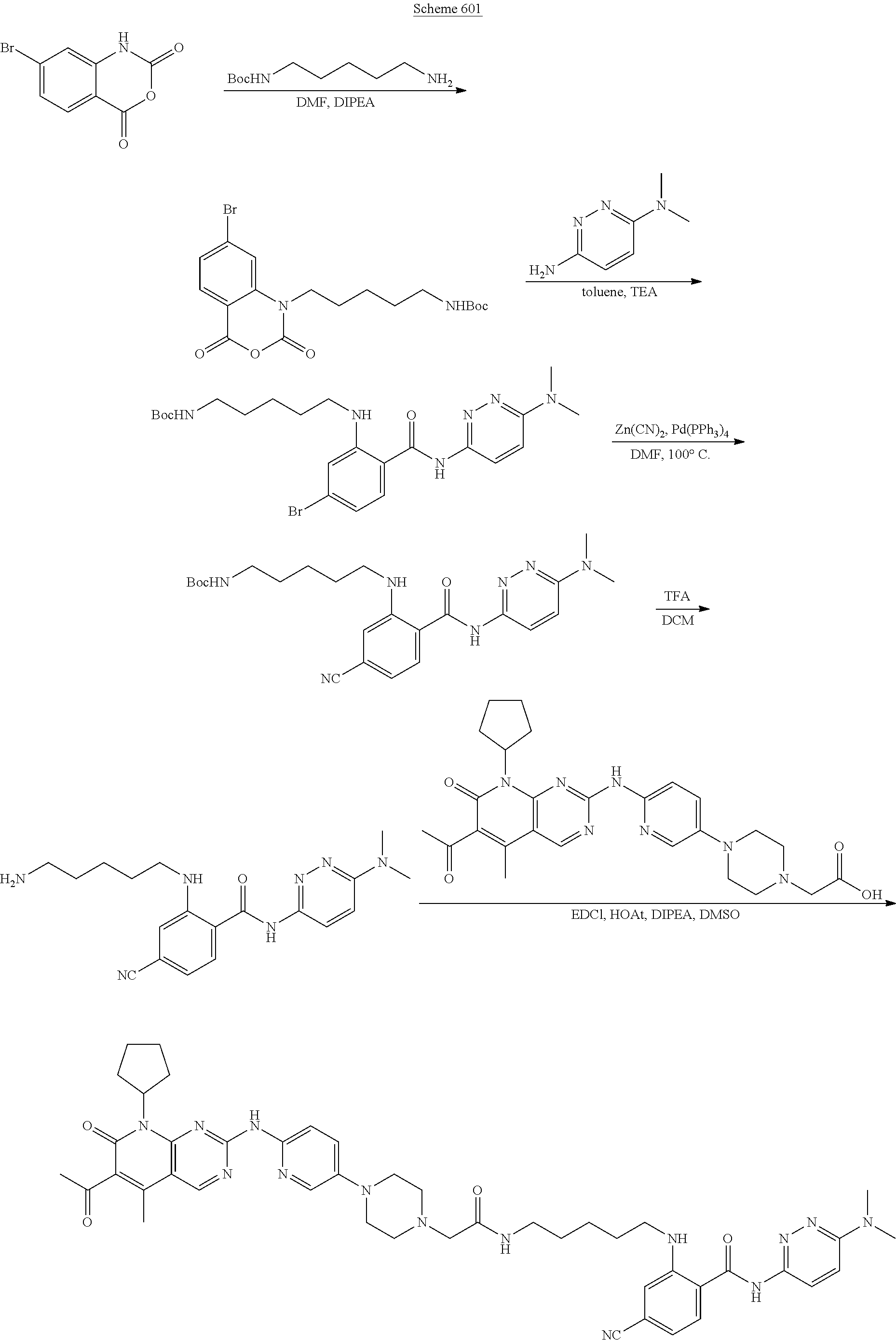

### Step 1. Synthesis of tert-butyl (5-(7-bromo-2,4-dioxo-2H-benzo[d][1,3]oxazin-1(4H)-yl)pentyl)carbamate

The title compound was synthesized following the procedure of step 1 for the preparation of BL1-238 (420 mg, 51% yield). MS (ESI) m/z=427.1 [M+H]⁺.

### Step 2. Synthesis of tert-butyl (5-((5-bromo-2-((6-(dimethylamino)pyridazin-3-yl)carbamoyl)phenyl)amino)pentyl)carbamate

The title compound was synthesized following the procedure of step 2 for the preparation of BL1-238 (150 mg, 60% yield). MS (ESI) m/z=521.4 [M+H]⁺.

### Step 3. Synthesis of 2-((5-aminopentyl)amino)-4-cyano-N-(6-(dimethylamino)pyridazin-3-yl)benzamide

To a solution of tert-butyl (5-((5-bromo-2-((6-(dimethylamino)pyridazin-3-yl)carbamoyl)phenyl)amino)pentyl)carbamate (30 mg, 0.058 mmol) in DMF (5 mL) were added zinc cyanide (27.0 mg, 0.23 mmol), Pd(PPh₃)₄ (6.65 mg, 0.0058 mmol) at rt under N₂. After the reaction mixture was stirred at 100° C. for 3 h, it was purified by reverse-phase chromatography to provide the desired product (25 mg, 67% yield) as a yellow solid. MS (ESI) m/z=468.6 [M+H]⁺.

The remaining steps were performed according to the standard procedures to provide the desired product (3.9 mg, 9% yield over 2 steps) as a yellow solid. MS (ESI) m/z=855.9 [M+H]⁺.

### Example 602. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-4-(methylamino)benzamide (CPD-377)

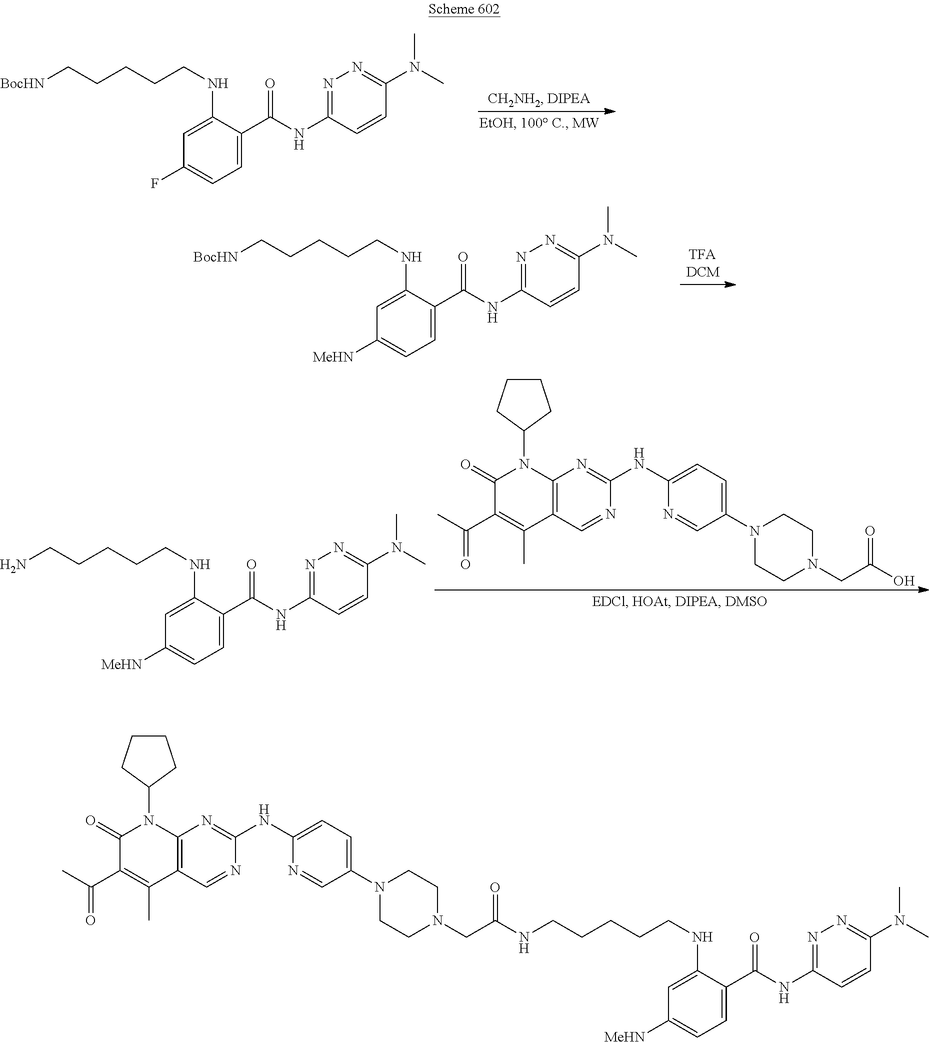

### Step 1. Synthesis of tert-butyl (5-((2-((6-(dimethylamino)pyridazin-3-yl)carbamoyl)-5-(methylamino)phenyl)amino)pentyl)carbamate

To a solution of tert-butyl (5-((2-((6-(dimethylamino)pyridazin-3-yl)carbamoyl)-5-fluorophenyl)amino)pentyl)carbamate (45 mg, 0.098 mmol) in EtOH (1 mL) were added MeNH₂·HCl (33 mg, 0.49 mmol) and DIPEA (126 mg, 0.98 mmol) at rt. The reaction mixture was stirred at 100° C. under microwave irradiation for 8 h. After cooling down to rt, the mixture was purified by reverse-phase chromatography followed by prep-HPLC to provide the desired product (7 mg, 12% yield) as a colorless oil. MS (ESI) m/z=472.7 [M+H]⁺.

The remaining steps were performed according to the standard procedure of CPD-341 to provide the desired product (3.9 mg, 23% yield over 2 steps) as a yellow solid. MS (ESI) m/z=859.8 [M+H]⁺.

### Example 603. 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-4-chloro-N-(6-cyclopropyl-5-methylpyridin-2-yl)benzamide (CPD-378)

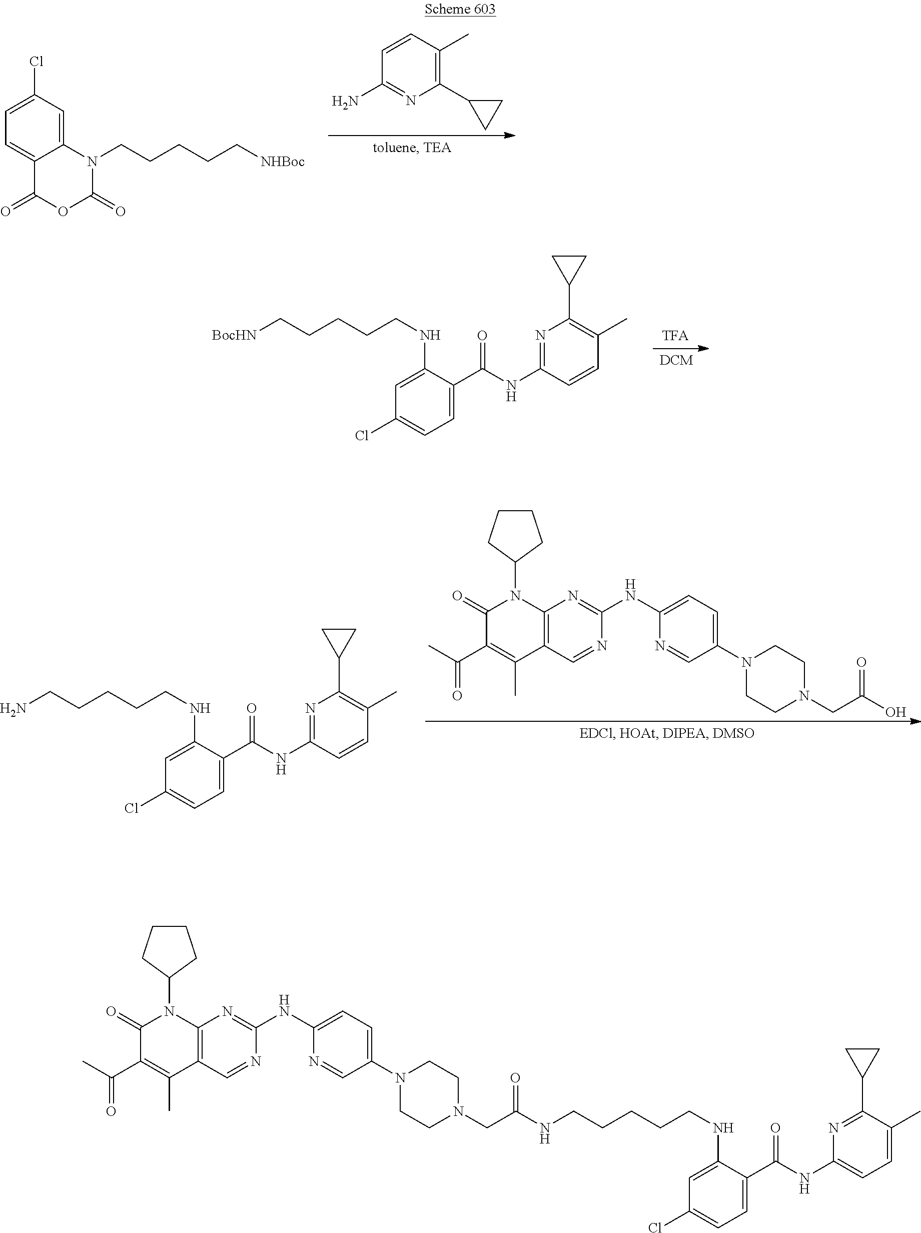 [SMILES: CC(C)(C)OC(=O)NCCCCCn1c(=O)oc(=O)c2ccc(Cl)cc21 ; Cc1ccc(N)nc1C1CC1 ; Cc1ccc(NC(=O)c2ccc(Cl)cc2NCCCCCNC(=O)OC(C)(C)C)nc1C1CC1 ; Cc1ccc(NC(=O)c2ccc(Cl)cc2NCCCCCN)nc1C1CC1 ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)NCCCCCNc5cc(Cl)ccc5C(=O)Nc5ccc(C)c(C6CC6)n5)CC4)cn3)nc2n(C2CCCC2)c1=O]

CPD-378 was synthesized following the standard procedures for preparing BL1-238 and CPD-341 (3.0 mg, 4% yield over 3 steps) as a yellow solid. MS (ESI) m/z=874.8 [M+H]⁺.

### Example 604. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)hexyl)amino)-4-chloro-N-(6-cyclopropyl-5-methylpyridin-2-yl)benzamide (CPD-379)

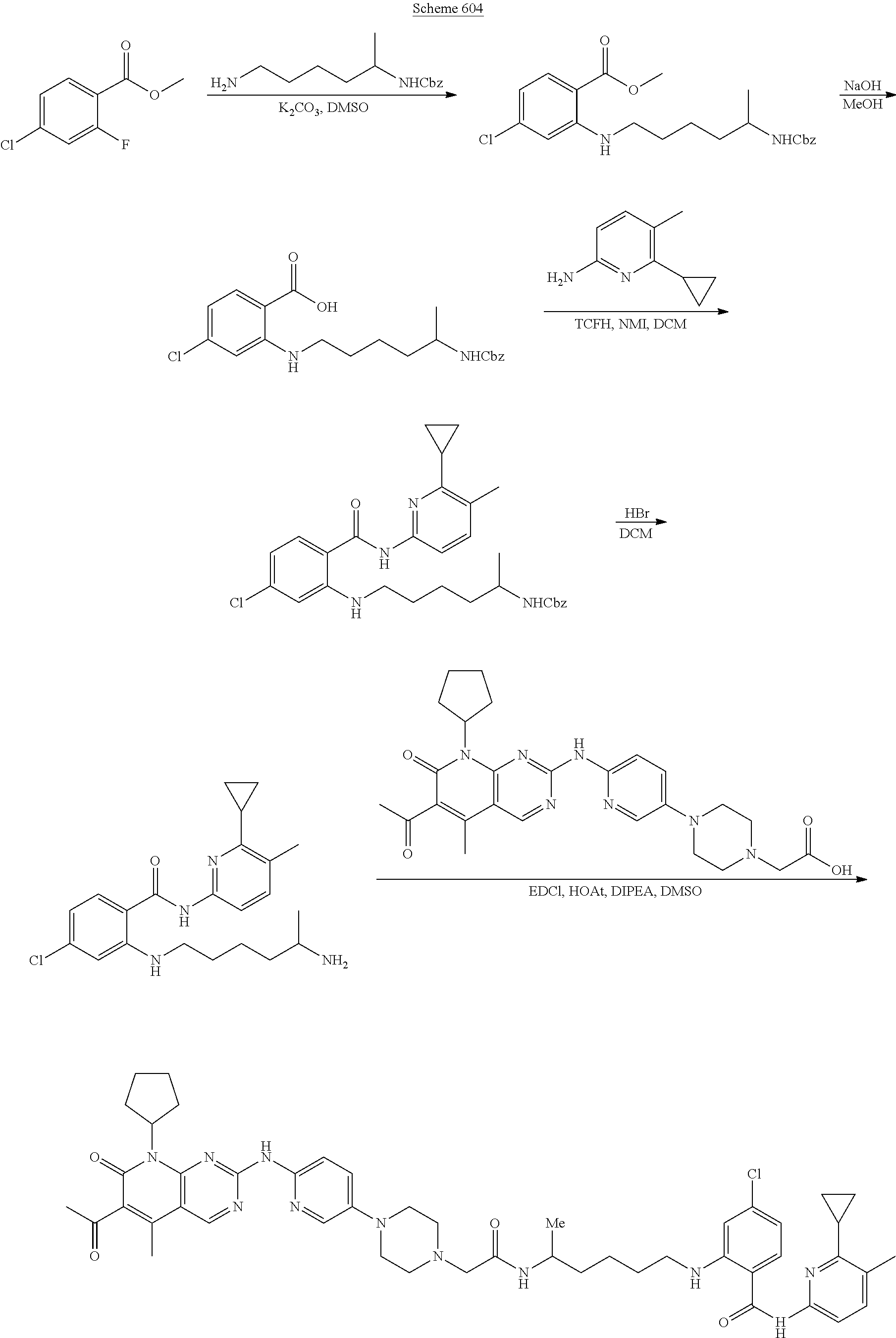

### Step 1. Synthesis of methyl 2-((5-(((benzyloxy)carbonyl)amino)hexyl)amino)-4-chlorobenzoate

To a solution of methyl 4-chloro-2-fluorobenzoate (50 mg, 0.3 mmol) and benzyl (6-aminohexan-2-yl)carbamate (66 mg, 0.26 mmol) in DMSO (5 mL) was added K₂CO₃ (112 mg, 0.81 mmol) at rt. The reaction mixture was stirred at 100° C. for 16 h. After cooling down to rt, the mixture was purified by reverse-phase chromatography to provide the desired product (52 mg, 46% yield) as a white solid. MS (ESI) m/z=419.6 [M+H]⁺.

### Step 2. Synthesis of 2-((5-(((benzyloxy)carbonyl)amino)hexyl)amino)-4-chlorobenzoic acid

A mixture of methyl 2-((5-(((benzyloxy)carbonyl)amino)hexyl)amino)-4-chlorobenzoate (52 mg, 0.12 mmol) and NaOH (15 mg, 0.36 mmol) in THF/MeOH/H₂O (4:2:1, 5 mL) was stirred at 80° C. for 3 h. The resulting mixture was concentrated and purified by reverse-phase chromatography to give the desired product (56 mg, 87% yield) as a light yellow solid. MS (ESI) m/z=405.6 [M+H]⁺.

### Step 3. Synthesis of benzyl (6-((5-chloro-2-((6-cyclopropyl-5-methylpyridin-2-yl)carbamoyl)phenyl)amino)hexan-2-yl)carbamate

A mixture of 2-((5-(((benzyloxy)carbonyl)amino)hexyl)amino)-4-chlorobenzoic acid (45 mg, 0.1 mmol), 6-cyclopropyl-5-methylpyridin-2-amine (16 mg, 0.11 mmol), TCFH (6 mg, 0.15 mmol) and NMI (27 mg, 0.33 mmol) in DCM (10 mL) was stirred at rt for 16 h. The reaction mixture was poured into water (50 mL) and extracted with ethyl acetate (3×20 mL). The combined organic layers were washed with saturated brine (20 mL), dried over anhydrous sodium sulfate, filtered and evaporated under reduced pressure. The resulting residue was purified by reverse-phase chromatography to provide the desired product (15 mg, 25% yield) as a light yellow solid. MS (ESI) m/z=535.7 [M+H]⁺.

### Step 4. Synthesis of 2-((5-aminohexyl)amino)-4-chloro-N-(6-cyclopropyl-5-methylpyridin-2-yl)benzamide

To a solution of benzyl (6-((5-chloro-2-((6-cyclopropyl-5-methylpyridin-2-yl)carbamoyl)phenyl)amino)hexan-2-yl)carbamate (15 mg, 0.03 mmol) in DCM (2 mL) was added HBr (1 mL, 48 wt %) at 0° C. After the reaction mixture was stirred at rt for 1 h, it was concentrated and purified by reverse-phase chromatography to provide the desired product (9 mg, 62% yield) as a white solid. MS (ESI) m/z=401.6 [M+H]⁺.

### Step 5. Synthesis of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)hexyl)amino)-4-chloro-N-(6-cyclopropyl-5-methylpyridin-2-yl)benzamide

A mixture of 2-((5-aminohexyl)amino)-4-chloro-N-(6-cyclopropyl-5-methylpyridin-2-yl)benzamide (9 mg, 0.02 mmol), 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid (12 mg, 0.02 mmol), EDCI (5.8 mg, 0.03 mmol), HOAt (4.2 mg, 0.03 mmol) and DIPEA (13 mg, 0.10 mmol) in DMSO (2 mL) was stirred at rt for 16 h. The reaction mixture was poured into water (10 mL) and extracted with ethyl acetate (3×20 mL). The combined organic layers were washed with saturated brine (20 mL), dried over anhydrous sodium sulfate, filtered and evaporated under reduced pressure. The resulting residue was purified by reverse-phase chromatography to provide the desired product (1.8 mg, 10% yield) as a yellow solid. MS (ESI) m/z=888.8 [M+H]⁺.

### Example 605. 3-((2-(2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)ethyl)amino)-N-(6-methoxypyridazin-3-yl)-2-methylbenzamide (CPD-380)

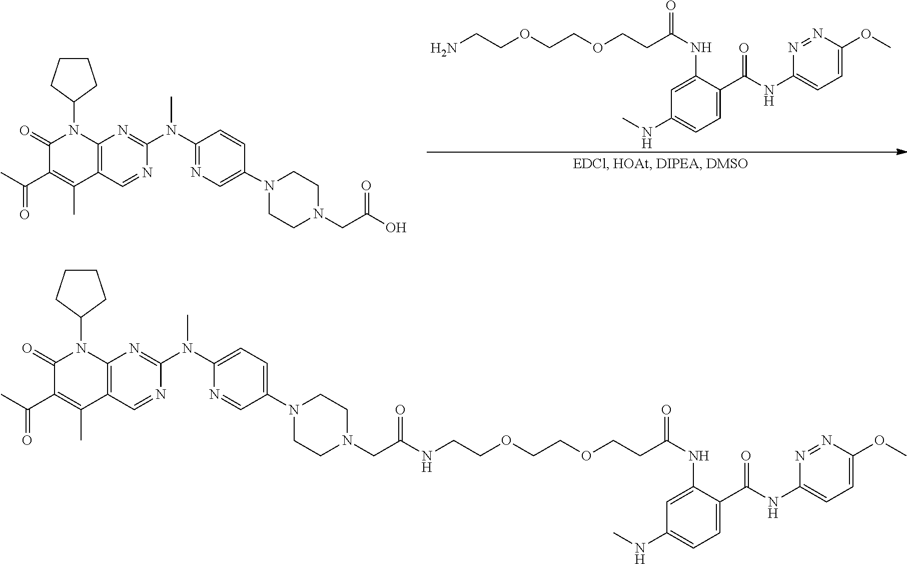 [SMILES: CC(=O)c1c(C)c2cnc(N(C)c3ccc(N4CCN(CC(=O)O)CC4)cn3)nc2n(C2CCCC2)c1=O ; CNc1ccc(C(=O)Nc2ccc(OC)nn2)c(NC(=O)CCOCCOCCN)c1 ; CNc1ccc(C(=O)Nc2ccc(OC)nn2)c(NC(=O)CCOCCOCCNC(=O)CN2CCN(c3ccc(N(C)c4ncc5c(C)c(C(C)=O)c(=O)n(C6CCCC6)c5n4)nc3)CC2)c1]

CPD-380 was synthesized following the standard procedure for preparing CPD-341 (3.3 mg, 34% yield) as a yellow solid. MS (ESI) m/z=934.9 [M+H]⁺.

### Example 606. CPD-004 and CPD-031 Bound to DDB1

The binding affinities of heterobifunctional compounds to DDB1 were determined by SPR assay (FIG. 1A-1B). Purified DDB1ΔBPB proteins were immobilized on a CM5 sensor chip, and a dose range of compound solutions were injected in multi-cycle kinetic format. Data were fit to a steady state model to provide equivalent dissociation constants (K_{d}). The SPR experiment showed that heterobifunctional compounds CPD-004 and CPD-031 bound to DDB1 in a concentration-dependent manner, and their binding affinities (K_{d}) were 9.4 M and 5.7 M, respectively.

### Example 607. SPR Binding Assay

The SPR binding affinity results of selected DDB1 ligands and heterobifunctional compounds are set forth in Table 5.

TABLE 5
Binding affinities to DDB1.

| Compound | Kd |
|---|---|
| B1-1 | D |
| B1-2 | D |
| B1-3 | C |
| B1-4 | A |
| B1-5 | B |
| B1-6 | B |
| B1-7 | D |
| B1-8 | D |
| B1-31 | D |
| B1-53 | B |
| B1-67 | B |
| B1-71 | B |
| B1-73 | D |
| B1-79 | B |
| B1-80 | A |
| B1-83 | B |
| B1-88 | A |
| BL1-24 | A |
| BL1-36 | A |
| BL1-38 | A |
| CPD-002 | B |
| CPD-004 | A |
| CPD-020 | A |
| CPD-021 | B |
| CPD-022 | A |
| CPD-023 | C |
| CPD-031 | A |
| CPD-033 | A |
| CPD-042 | A |
| CPD-043 | A |
| CPD-044 | A |
| CPD-046 | A |
| CPD-047 | A |
| CPD-049 | B |
| CPD-114 | A |
| CPD-115 | A |

The binding affinities to DDB1 were determined by SPR assay. A: Kd < 20 uM; B: 20 uM < Kd < 50 uM; C: 50 uM < Kd < 100 uM; D: Kd > 100 uM.

### Example 608. CPD-002 and CPD-004 Concentration-Dependently Reduced Cyclin D1, Cyclin D2, Cyclin D3, CDK4 and CDK6 Protein Levels

Calu-1 (FIG. 2A) or BT-549 (FIG. 2B) cells were treated with CPD-002, CPD-004, or palbociclib at indicated concentrations for 16 hours. CPD-002 and CPD-004 reduced cyclin D1, cyclin D2, cyclin D3, CDK4 and CDK6 protein levels in a concentration-dependent manner in both cell lines. In contrast, palbociclib didn't significantly change the levels of these proteins in either cell lines. In addition, CPD-002 and CPD-004 also inhibited downstream Rb phosphorylation and induced cleaved caspase-3 in a concentration-dependent manner.

### Example 609. CPD-031 Concentration-Dependently Reduced Cyclin D1, Cyclin D2, Cyclin D3, CDK4 and CDK6 Protein Levels (FIG. 3)

Calu-1 cells were treated with CPD-031 at indicated concentrations for 16 hours. CPD-031 downregulated the protein levels of cyclin D1, cyclin D2, cyclin D3, CDK4, CDK6, and downstream phosphorylated Rb in a concentration-dependent manner.

### Example 610. CPD-002 and CPD-031 Rapidly Reduced Cyclin D Protein Levels (FIG. 4A-4B)

Calu-1 cells were treated with CPD-002 or CPD-031 at 500 nM or 100 nM, respectively for indicated period of time. Data showed that cyclin D1, cyclin D2 and cyclin D3 protein levels were significantly reduced as early as one hour following treatment, while CDK4 and CDK6 were reduced much slower compared to cyclin D.

### Example 611. Heterobifunctional Compound-Mediated Degradation of Cyclin D Depended on the Ubiquitin-Proteasome System and E3 Ligase DDB1

Calu-1 cells were pre-treated with DMSO, 20 μM MG-132 (MG), 5 μM MLN4924 (MLN) or 1 μM TAK-243 (TAK) for 2 hours, and subsequently incubated with 500 nM CPD-002, 500 nM CPD-004, or 100 nM CPD-031 for another 4 h or 2 h prior to immunoblotting (FIG. 5A-5B). Data showed that pretreatment with the proteasome inhibitor MG-132, the cullin E3 ligase inhibitor MLN4924, or the ubiquitin activating enzyme (UAE) inhibitor TAK-243 diminished the cyclin D degradation effect of CPD-002, CPD-004, and CPD-031.

Parental and DDB1 knockout Hs578T cells were treated with a dose range of CPD-031 for 4 hours (FIG. 5C). Data showed that CPD-031-mediated cyclin D1 degradation is partially compromised by depletion of DDB1 E3 ligase.

### Example 612. Heterobifunctional Compound-Mediated Cyclin D Degradation Depended on Binding to the Target Protein (FIG. 6A-6D)

Calu-1 cells were treated with a dose range of negative control compounds CPD-042 or CPD-049 for 16 hours (FIG. 6A-6B). Immunoblotting data showed that these two negative control compounds showed much weaker degradation potencies compared with their corresponding active heterobifunctional compounds (FIG. 2A and FIG. 3).

Calu-1 cells were seeded in 96-well plates and treated with CPD-002, CPD-042, CPD-031, or CPD-049 following a 9-point serial dilution for 3 days (FIG. 6C-6D). Cell viability data showed that the negative control compounds CPD-042 and CPD-049 showed much weaker cellular anti-proliferation activities compared to CPD-002 and CPD-031, respectively.

### Example 613. Heterobifunctional Compound-Mediated Cyclin D Degradation Depended on Binding to the Target Protein (FIG. 10A-10B)

T47D cells were treated with a dose range of heterobifunctional compound CPD-343 or its negative control compound CPD-380 for 48 hours. Immunoblotting data showed that CPD-380 showed much weaker degradation potencies compared with its corresponding active heterobifunctional compound (FIG. 10A).

T47D cells were seeded in 96-well plates and treated with of CP-343, or CPD-380 following a 10-point serial dilution for 6 days. Cell viability data showed that negative control compound CPD-380 showed much weaker cellular anti-proliferation activities compared to its corresponding active heterobifunctional compound CPD-343 (FIG. 10B).

### Example 614. CRBN-Recruiting Heterobifunctional Compound CP-10 and BSJ-03-123 Didn't Reduce Cyclin D1 Protein Levels or Suppress Cell Growth in Calu-1 Cells (FIG. 11A-11B)

Calu-1 cells were treated with reference compounds CP-10 or BSJ-03-123 at indicated concentrations for 8 hours. Immunoblotting data showed that these two compounds significantly reduced CDK4 and CDK6 protein levels, but didn't affect cyclin DC protein levels (FIG. 11A).

Calu-1 cells were seeded in 96-well plates and treated with reference compounds CP-10 or BSJ-03-123 following a 11-point serial dilution for 3 days. Cell viability data showed that these two compounds didn't significantly suppress tumor cell viability (FIG. 11B).

### Example 615. Heterobifunctional Compounds Reduced Cyclin D1 and CDK4 Protein Levels

The cellular protein degradation results of selected heterobifunctional compounds are set forth in Tables 6A and 6B.

TABLE 6A
Cyclin D1 and CDK4 percentage degradation in Calu-1 Cell.

| | CyclinD1 | CDK4 |
|---|---|---|
| | Degradation | Degradation |
| Compound | (200 nM) | (200 nM) |
| CPD-001 | D | D |
| CPD-002 | A | B |
| CPD-003 | D | D |
| CPD-004 | A | C |
| CPD-005 | D | D |
| CPD-006 | D | D |
| CPD-007 | D | D |
| CPD-008 | D | D |
| CPD-009 | D | D |
| CPD-010 | D | D |
| CPD-011 | D | D |
| CPD-012 | D | D |
| CPD-013 | D | D |
| CPD-014 | D | D |
| CPD-015 | D | D |
| CPD-016 | D | D |
| CPD-017 | D | D |
| CPD-018 | C | C |
| CPD-019 | D | D |
| CPD-024 | D | D |
| CPD-025 | D | D |
| CPD-026 | B | C |
| CPD-027 | D | D |
| CPD-028 | D | D |
| CPD-029 | D | D |
| CPD-030 | B | C |
| CPD-031 | A | A |
| CPD-032 | D | D |
| CPD-033 | A | A |
| CPD-034 | D | D |
| CPD-035 | C | C |
| CPD-036 | D | D |
| CPD-037 | D | D |
| CPD-038 | D | D |
| CPD-039 | D | D |
| CPD-040 | D | D |
| CPD-041 | D | D |
| CPD-042 | D | D |
| CPD-043 | A | A |
| CPD-044 | A | B |
| CPD-045 | A | B |
| CPD-046 | A | A |
| CPD-047 | A | A |
| CPD-048 | D | D |
| CPD-049 | D | D |
| CPD-050 | A | A |
| CPD-051 | A | A |
| CPD-052 | A | B |
| CPD-053 | B | B |
| CPD-054 | A | B |
| CPD-055 | D | D |
| CPD-056 | A | B |
| CPD-057 | A | B |
| CPD-058 | D | D |
| CPD-059 | D | D |
| CPD-060 | A | B |
| CPD-061 | A | B |
| CPD-062 | D | D |
| CPD-063 | D | D |
| CPD-064 | D | D |
| CPD-065 | A | B |
| CPD-066 | D | D |
| CPD-067 | A | B |
| CPD-068 | A | B |
| CPD-069 | D | D |
| CPD-070 | A | A |
| CPD-071 | A | A |
| CPD-072 | D | B |
| CPD-073 | D | B |
| CPD-074 | B | B |
| CPD-075 | A | A |
| CPD-076 | C | B |
| CPD-077 | D | B |
| CPD-078 | D | D |
| CPD-079 | D | D |
| CPD-080 | D | D |
| CPD-081 | A | A |
| CPD-082 | A | A |
| CPD-083 | A | B |
| CPD-084 | A | B |
| CPD-085 | A | A |
| CPD-086 | D | D |
| CPD-087 | D | D |
| CPD-088 | D | D |
| CPD-089 | B | B |
| CPD-090 | A | B |
| CPD-091 | D | D |
| CPD-092 | D | D |
| CPD-093 | D | D |
| CPD-094 | D | D |
| CPD-095 | D | D |
| CPD-096 | D | D |
| CPD-097 | A | B |
| CPD-098 | D | D |
| CPD-099 | D | D |
| CPD-100 | D | D |
| CPD-101 | A | B |
| CPD-102 | A | B |
| CPD-103 | D | D |
| CPD-104 | A | C |
| CPD-105 | A | B |
| CPD-106 | A | B |
| CPD-107 | A | A |
| CPD-108 | D | D |
| CPD-109 | D | D |
| CPD-110 | D | D |
| CPD-111 | D | D |
| CPD-112 | D | D |
| CPD-113 | A | B |
| CPD-114 | A | A |
| CPD-115 | A | A |
| CPD-116 | D | D |
| CPD-117 | C | C |
| CPD-118 | D | D |
| CPD-119 | D | D |
| CPD-120 | D | D |
| CPD-121 | C | C |
| CPD-122 | A | B |
| CPD-123 | A | B |
| CPD-124 | D | D |
| CPD-125 | D | D |
| CPD-126 | B | B |
| CPD-127 | C | C |
| CPD-128 | C | B |
| CPD-129 | D | D |
| CPD-130 | D | D |
| CPD-131 | D | D |
| CPD-132 | A | A |
| CPD-133 | A | B |
| CPD-134 | D | D |
| CPD-135 | D | D |
| CPD-136 | A | B |
| CPD-137 | D | D |
| CPD-138 | D | D |
| CPD-139 | D | D |
| CPD-140 | A | B |
| CPD-141 | A | B |
| CPD-142 | D | D |
| CPD-143 | A | A |
| CPD-144 | A | B |
| CPD-145 | D | D |
| CPD-146 | D | D |
| CPD-147 | D | D |
| CPD-148 | D | D |
| CPD-149 | A | A |
| CPD-150 | A | B |
| CPD-151 | C | B |
| CPD-152 | A | B |
| CPD-153 | D | D |
| CPD-154 | D | D |
| CPD-155 | A | B |
| CPD-156 | D | D |
| CPD-157 | D | D |
| CPD-158 | D | D |
| CPD-159 | D | C |
| CPD-160 | C | B |
| CPD-161 | D | D |
| CPD-162 | C | D |
| CPD-163 | A | B |
| CPD-164 | A | B |
| CPD-165 | D | D |
| CPD-166 | B | C |
| CPD-167 | D | D |
| CPD-168 | A | B |
| CPD-169 | D | D |
| CPD-170 | A | B |
| CPD-171 | D | D |
| CPD-172 | D | C |
| CPD-173 | A | A |
| CPD-174 | A | A |
| CPD-175 | A | A |
| CPD-176 | A | A |
| CPD-177 | D | D |
| CPD-178 | D | D |
| CPD-179 | D | D |
| CPD-180 | D | D |
| CPD-181 | D | D |
| CPD-182 | D | D |
| CPD-183 | D | D |
| CPD-184 | A | B |
| CPD-185 | A | A |
| CPD-186 | B | B |
| CPD-187 | D | D |
| CPD-188 | D | D |
| CPD-267 | A | B |
| CPD-268 | A | B |
| CPD-269 | B | B |
| CPD-270 | A | B |
| CPD-271 | D | D |
| CPD-328 | D | D |
| CPD-329 | A | A |
| CPD-337 | D | D |
| CPD-338 | A | B |
| CPD-339 | A | A |
| CPD-340 | D | D |
| CPD-341 | D | D |
| CPD-342 | A | B |
| CPD-343 | A | A |
| CPD-344 | A | B |
| CPD-345 | A | B |
| CPD-346 | A | B |
| CPD-347 | D | D |
| CPD-348 | D | D |
| CPD-350 | A | B |
| CPD-351 | D | D |
| CPD-357 | A | B |
| CPD-358 | A | B |
| CPD-359 | A | B |
| CPD-360 | A | A |
| CPD-361 | A | B |
| CPD-362 | D | D |
| CPD-363 | A | B |
| CPD-364 | A | B |
| CPD-365 | A | A |
| CPD-366 | D | D |
| CPD-367 | D | D |
| CPD-368 | A | A |
| CPD-369 | D | D |
| CPD-370 | A | A |
| CPD-371 | A | B |
| CPD-372 | A | B |
| CPD-373 | A | B |
| CPD-374 | A | B |
| CPD-375 | A | B |
| CPD-376 | A | B |
| CPD-377 | A | B |
| CPD-378 | B | D |
| CPD-379 | A | B |

Calu-1 Cells were treated with heterobifunctional compounds at 200 nM for 16 hours. A: protein percentage degradation >=80%; B: protein percentage degradation <80%, and >=50%; C: protein percentage degradation <50%, and >=30%; D: protein percentage degradation <30%.

TABLE 6B
Cyclin D1 and CDK4 percentage degradation in Calu-1 Cell.

| | CyclinD1 | CDK4 |
|---|---|---|
| | Degradation | Degradation |
| Compound | (10 μM) | (10 μM) |
| CPD-020 | D | D |
| CPD-021 | A | B |
| CPD-022 | A | A |
| CPD-023 | A | A |
| CPD-272 | D | D |
| CPD-273 | D | D |
| CPD-274 | B | D |
| CPD-275 | D | D |
| CPD-276 | D | D |
| CPD-277 | D | D |
| CPD-278 | D | D |
| CPD-279 | D | D |
| CPD-280 | D | D |
| CPD-281 | D | D |
| CPD-282 | D | D |
| CPD-283 | D | D |
| CPD-284 | D | D |
| CPD-285 | D | D |
| CPD-286 | A | B |
| CPD-287 | A | C |
| CPD-288 | A | B |
| CPD-289 | A | C |
| CPD-290 | B | C |
| CPD-291 | B | C |
| CPD-292 | B | D |
| CPD-293 | A | B |
| CPD-294 | A | B |
| CPD-295 | A | B |
| CPD-296 | A | B |
| CPD-297 | A | B |
| CPD-298 | A | B |
| CPD-299 | B | C |
| CPD-300 | D | D |
| CPD-301 | D | D |
| CPD-302 | D | D |
| CPD-303 | D | D |
| CPD-304 | D | D |
| CPD-305 | D | D |
| CPD-306 | D | D |
| CPD-307 | D | D |
| CPD-308 | B | D |
| CPD-309 | D | C |
| CPD-310 | A | D |
| CPD-311 | A | C |
| CPD-312 | A | D |
| CPD-313 | A | D |
| CPD-314 | D | D |
| CPD-315 | D | D |
| CPD-316 | D | D |
| CPD-317 | A | B |
| CPD-318 | D | C |
| CPD-319 | A | B |
| CPD-320 | A | B |
| CPD-321 | A | B |
| CPD-322 | A | B |
| CPD-323 | A | B |
| CPD-324 | A | B |
| CPD-325 | A | B |
| CPD-326 | A | B |
| CPD-327 | A | B |

Calu-1 Cells were treated with heterobifunctional compounds at 10 μM for 16 hours. A: protein percentage degradation >= 80%; B: protein percentage degradation < 80%, and >= 50%; C: protein percentage degradation < 50%, and >= 30%; D: protein percentage degradation < 30%.

### Example 616. CPD-002 and CPD-031 Suppressed Cell Viability Across Multiple Cancer Types (FIG. 7 and Table 7)

Cells were treated with CDK4/6 inhibitors palbociclib, ribociclib, or abemaciclib, or heterobifunctional compounds CPD-002 or CPD-031 following a 9-point serial dilution for 3 days. Heterobifunctional compounds showed significant advantages over CDK4/6 inhibitors by targeting a broad set of cancer cell lines

The cell viability inhibition results of selected heterobifunctional compounds and FDA-approved CDK4/6 inhibitors are set forth in Table 7. Additional data is shown in FIG. 7.

TABLE 7
Cellular Anti-proliferation activity (IC₅₀, nM)
of selected compounds in different cell line.

| Cell line | Tumor | | | | | |
|---|---|---|---|---|---|---|
| (IC₅₀, nM) | type | CPD-031 | CPD-002 | palbociclib | ribociclib | abemaciclib |
| Calu-1 | NSCLC | 9 | 68 | >10000 | >10000 | 29000 |
| NCI-H522 | NSCLC | 155 | 638 | >10000 | >10000 | >3000 |
| MDA-MB-157 | TNBC | 28 | 151 | >10000 | >10000 | >10000 |
| MDA-MB-453 | TNBC | 300 | 741 | 2700 | >3000 | 30 |
| MDA-MB-468 | TNBC | | 1480 | >10000 | >10000 | 3090 |
| Hs578T | TNBC | 38 | 215 | >10000 | >10000 | 1200 |
| BT-549 | TNBC | 110 | 690 | >10000 | >10000 | 2070 |
| KURAMOCHI | OC | 190 | 920 | >10000 | >10000 | 2500 |
| OVCAR3 | OC | 39 | 107 | >10000 | >10000 | >10000 |
| MIA PaCa-2 | PC | 57 | 175 | >3000 | >3000 | 550 |
| Cal-62 | TC | 5 | 50 | >10000 | >10000 | 2120 |

The IC₅₀ value of each compound was determined and calculated using the GraphPad Prism 5.0 software.
NSCLC: non-small cell lung cancer, TNBC: triple-negative breast cancer, OC: ovarian cancer, PC: pancreatic cancer, TC: thyroid cancer.

### Example 617. CPD-343 Caused Cell Apoptosis in ER⁺ Breast Cancer T47D Cells (FIG. 12)

T47D cells were treated with DMSO, palbociclib, heterobifunctional compound CPD-343, or negative control compound CPD-380 for 6 days at doses approximating IC₅₀ and IC₉₀ concentrations as indicated. Cells were harvested by trypsinization, staining was carried out using the Annexin V Apoptosis Detection Kit. Flow cytometric analysis showed that CPD-343 cause significant cell apoptosis (Annexin V⁺ population, 26.9% at 10 nM; 52.6% at 200 nM), while palbociclib or CPD-380 showed much less effect on cell apoptosis.

### Example 618. CPD-343 Suppressed Cell Viability in T47D Palbo-Resistant Model (FIG. 13)

T47D cells were cultured long-term to resistance in the presence of 1 μM Palbocicilib. Palbo resistance was determined by CellTiter-Lumi cell viability assay. T47D parental or palbo-resistant cells were treated with palbociclib, or heterobifunctional compound CPD-343 following a 10-point serial dilution for 6 days. Cell viability data showed that CPD-343 remained effective in T47D palbo-resistant model.

### Example 619. CPD-191 Concentration-Dependently Reduced P300 and CBP Protein Levels in Multiple Cell Lines (FIG. 8)

LNCaP, Calu-1, NCI-H1703, or MM.1R cells were treated with CPD-191 at indicated concentrations for 8 hours. Heterobifunctional compound CPD-191 reduced P300 and CBP protein levels in a concentration-dependent manner in multiple cell lines.

### Example 620. CPD-253 Concentration-Dependently Reduced BRD4 Protein Levels in Multiple Cell Lines (FIG. 9)

Daudi, SU-DHL-4, or MDA-MB-231 cells were treated with CPD-253 at indicated concentrations for 8 hours. Heterobifunctional compound CPD-253 reduced BRD4 protein levels in a concentration-dependent manner in multiple cell lines.

### Example 621. Materials and Methods of Experiments Described Herein

### Antibody and Reagent

Anti-cyclin D1 (2978S), anti-cyclin D2 (3741S), anti-cyclin D3 (29365), anti-CDK4 (12790S), anti-CDK6 (3136S), anti-phospho-R^{b} (8516S), anti-cleaved Caspase-3 (9664S), anti-FoxM1 (5436S), anti-cyclin A2 (4656S), anti-P300 antibody (86377S), anti-CBP antibody (7389S), anti-vinculin antibody (18799S), and anti-BRD4 (13440S) antibodies were purchased from Cell Signaling Technology. Anti-DDB1 antibody (ab109027) was purchased from Abcam. HRP-conjugated anti-α-tubulin antibody and anti-α-GAPDH antibody were purchased from GNI. Media, and other cell culture reagents were purchased from Thermo Fisher Scientific. CellTiter-Glo Luminescent Assay kit was purchased from Promega.

### Cell Culture and Transfection

Calu-1, T47D, MCF7, NCI-H522, BT-549, MDA-MB-157, MDA-MB-453, MDA-MB-468, Hs578T, KURAMOCHI, OVCAR3, MIA PaCa-2, Cal-62, LNCaP, NCI-H1703, MM.1R, Daudi, SU-DHL-4, MDA-MB-231 and other cells were cultured at 37° C. with 5% CO₂ in RPMI 1640 or DMEM medium supplemented with 10% fetal bovine serum. Cells were authenticated using the short tandem repeat (STR) assays. Mycoplasma test results were negative. Cell transfection was performed using PEI or Lipofectamine 2000 (Invitrogen) following the manufacturer's instructions. For palbociclib-resistant cell models, T47D cells were cultured long-term to resistance in the presence of 1 μM palbociclib. Cells were deemed resistant when growing in the presence of palbociclib at the same rate as parental cells.

### CRISPR-Cas9 Mediated Knock-Out

The procedures for CRISPR-Cas9 mediated knock-out followed the previously published protocols (Ran et al., 2013). The sgRNA targeting human DDB1 (sgRNA sequence: CGATTAGGGTCAGACCGCAG) (SEQ ID NO: 1) was designed using the online CRISPR Design Tool (chopchop.cbu.uib.no/), and constructed into CRISPR-Cas9 vector, pLentiCRISPR V2 (Addgene #52961). Lentivirus was produced in HEK293T cells by co-transfecting pLentiCRISPR construct with packing vectors. Hs578T cells stably expressing Cas9 enzyme and sgRNA were established by lentivirus transduction, selected, and maintained in medium containing 1 g/mL puromycin.

### Immunoblotting

Cultured cells were washed with cold PBS once and lysed in cold RIPA buffer supplemented with protease inhibitors and phosphatase inhibitors (Beyotime Biotechnology). The solutions were then incubated at 4° C. for 30 min with gentle agitation to fully lysed cells. Cell lysates were centrifuged at 13,000 rpm for 10 min at 4° C. and pellets were discarded. Total protein concentrations in the lysates were determined following BCA assays (Beyotime Biotechnology). Cell lysates were mixed with Laemmli loading buffer and heated at 99° C. for 5 min. Proteins were resolved on SDS-PAGE and visualized using Western ECL Substrate kits on a ChemiDoc MP Imaging system (Bio-Rad). Protein bands were quantitated using the Image Lab software provided by Bio-Rad.

### Protein Expression and Purification

Human DDB1ΔBPB (UniProt: Q16531. BPB domain, aa 396 to 705, is replaced with a GNGNSG linker) coding sequences were cloned into pFastBacHTB vector and were expressed in SF9 cells using Bac-to-Bac baculovirus expression system (Thermo Fisher Scientific). The expression constructs include an N-terminal His6-tag to facilitate the purification, and a TEV protease cleavage site in between. DDB1ΔBPB proteins were obtained from supernatant of cell lysates and purified through sequential application of Ni affinity column (Ni-NTA column, Bio-Rad) and size-exclusion column (Superdex 200 16/600GL column, GE Healthcare) chromatography. Protein tags were cleaved off by TEV protease, and the resulting untagged proteins were further purified by Ni affinity column. Purified proteins were verified by immunoblotting, analytic SEC, and LC-MS.

### Surface Plasmon Resonance (SPR) Binding Assay

SPR studies were performed on a Biacore X100 plus instrument (GE Healthcare). Immobilization of purified DDB1ΔBPB was carried out at 25° C. using a CM5 sensor chip. The surface was pre-equilibrated in HBS-EP running buffer (10 mM HEPES, pH7.4, 150 mM NaCl, 3 mM EDTA, 0.05% P20), before it was activated with EDC/NHS. DDB1ΔBPB proteins were immobilized by amine coupling to a density of 10,000-12,000 resonance units (RUs) on flow cell FC2, whereas flow cell FC1 was used as reference. Both protein immobilized and reference surfaces were deactivated with 1M ethanolamine. All interaction experiments were performed at 25° C. Test compounds were prepared and serially diluted in HBS-EP running buffer containing final 5% DMSO (6-point two-fold serial dilution, 100 μM-3.125 μM final concentration of compounds). Compound solutions were injected individually in multi-cycle kinetic format without regeneration (flow rate 30 μl/min, association time 120 s, dissociation time 120 s). Sensorgrams from reference surfaces and blank injections were subtracted from the raw data (double-referencing) and the data was solvent-corrected prior to analysis. All data were fit to steady state affinity model using Biacore Evaluation Software to provide equivalent dissociation constants (K_{d}).

### Cell Viability Assay

Cells were seeded at a density of 2,000-5,000 cells per well in 96-well assay plates and treated with test compounds following a 10-point serial dilution for 3-6 days. Cell viability was determined using the CellTiter-Lumi assay kit according to the manufacturer's instructions. The dose-response curves were determined and IC₅₀ values were calculated using the GraphPad Prism software following a nonlinear regression (least squares fit) method. Data presented was mean±standard deviation (SD) unless otherwise indicated.

### Flow Cytometry

Assays were performed on the CytoFLEX Cytometer (Beckman Coulter), and data were analyzed using CytExpert software. For cell death and apoptosis analysis, cells were harvested by trypsinization, staining was carried out using the Annexin V Apoptosis Detection Kit (BD Biosciences). Briefly, cells were resuspended in 1× binding buffer and incubated with fluorochrome-conjugated Annexin V and 7-AAD for 15 min in darkness at room temperature.

### Example 622. P300 Degradation

The cellular protein degradation results of selected heterobifunctional compounds are set forth in Table 8.

TABLE 8
P300 Degradation

| | P300/CBP |
|---|---|
| | Degradation |
| Cpd. No. | (100 nM) |
| CPD-189 | D |
| CPD-190 | C |
| CPD-191 | A |
| CPD-192 | D |
| CPD-193 | D |
| CPD-194 | D |
| CPD-195 | D |
| CPD-196 | D |
| CPD-197 | D |
| CPD-198 | D |
| CPD-199 | D |
| CPD-200 | D |
| CPD-201 | D |
| CPD-202 | D |
| CPD-203 | D |
| CPD-204 | D |
| CPD-205 | D |
| CPD-206 | D |
| CPD-207 | D |
| CPD-208 | D |
| CPD-209 | D |
| CPD-210 | D |
| CPD-211 | D |
| CPD-212 | D |
| CPD-213 | D |
| CPD-214 | D |
| CPD-215 | D |
| CPD-216 | D |
| CPD-217 | D |
| CPD-218 | D |

A: degradation activity >= 80%; B: degradation activity < 80%, and >= 50%; C: B: degradation activity < 50%, and >= 30%; D: degradation activity < 30%.

While preferred embodiments of the present invention have been shown and described herein, variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A heterobifunctional compound of Formula (I), or a pharmaceutically acceptable salt thereof: 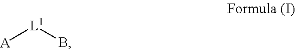
wherein,
A is a target protein binding moiety;
L¹ is a linker; and
B is a DDB1 binding moiety having the structure of Formula (II): 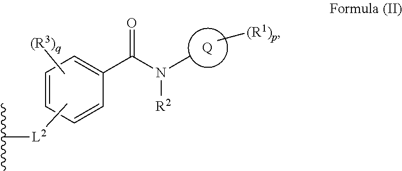
wherein,
ring Q is phenyl or a 5 or 6-membered monocyclic heteroaryl;
L² is a bond, —O—, —NR^{4A}—, —NR^{4B}—C(═O)—, —NR^{4B}—C(═O)—(C₁-C₃alkylene)-NR^{4A}—, —NR^{4B}—C(═O)—(C₁-C₃alkylene)-O—, —(C₁-C₃alkylene)-NR^{4B}—C(═O)—, —C(═O)NR^{4A}—, —C₁-C₃alkylene-, —C₂-C₃ alkenylene-, —C₂-C₃alkynylene-, C₃-C₈ cycloalkylene, or C₂-C₈ heterocyclene;
each R¹ is independently hydrogen, halogen, —CN, NO₂, —OR^{4A}, —NR^{4A}R^{4B}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl or heteroaryl, or
two R¹, together with the atom(s) to which they are connected, optionally form C₃-C₁₃ cycloalkyl, C₃-C₁₂ heterocyclyl, aryl, or heteroaryl;
R² is hydrogen, C₁-C₆ alkyl, C₃-C₈ cycloalkyl, OH, or O—C₁-C₄ alkyl;
each R³ is independently hydrogen, halogen, —CN, —NO₂, —OR^{4A}, —NR^{4A}R^{4B}, —C(═O)R^{4A}, —C(═O)OR^{4A}, —C(═O)NR^{4B}R^{4A}, —OC(═O)R^{4A}, —N(R^{4A})C(═O)R^{4B}, C₁-C₆ alkyl, C₁-C₆ haloalkyl, C₁-C₆ heteroalkyl, C₃-C₈ cycloalkyl, C₂-C₈ heterocyclyl, aryl, or heteroaryl, or two R³, together with the atom(s) to which they are connected, optionally form C₃-C₁₃ cycloalkyl, C₂-C₁₂ heterocyclyl, aryl, or heteroaryl;
each R^{4A} and R^{4B} is independently hydrogen, C₁-C₆ alkyl, C₂-C₆ alkenyl, C₂-C₆ alkynyl, C₁-C₆ haloalkyl, C₁-C₆ heteroalkyl, C₃-C₈ cycloalkyl, C₂-C₈ heterocyclyl, aryl, or heteroaryl, or
R^{4A} and R^{4B}, together with the atom(s) to which they are connected, optionally form C₂-C₁₂ heterocyclyl;
p is 1, 2 or 3; and
q is 1, 2 or 3.

2. The heterobifunctional compound of claim 1, or a pharmaceutically acceptable salt thereof, wherein ring Q is a 5-membered monocyclic heteroaryl.

3. The heterobifunctional compound of claim 2, or a pharmaceutically acceptable salt thereof, wherein the 5-membered monocyclic heteroaryl is pyrrolyl, furanyl, imidazolyl, pyrazolyl, oxazolyl, isoxazolyl, thienyl, thiazolyl, isothiazolyl, triazolyl, oxadiazolyl, thiadiazolyl, or tetrazolyl.

4. The heterobifunctional compound of any one of claims 1-3, or a pharmaceutically acceptable salt thereof, wherein the DDB1 binding moiety of Formula (II) has the structure of Formula (III-1) or (III-2): 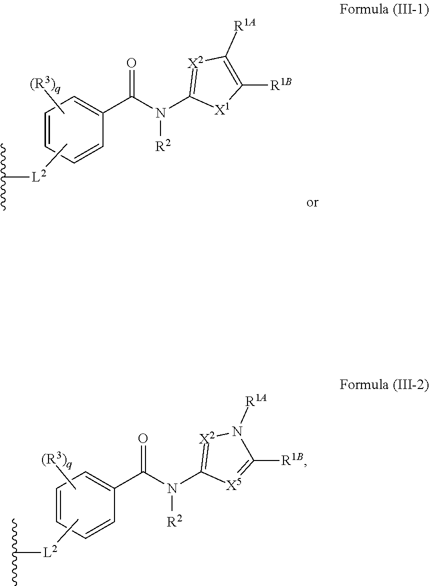
wherein,
X¹ is O, S, or NR⁵;
X² and X⁵ are independently N or CH;
R⁵ is hydrogen, C₁-C₆ alkyl, C₁-C₆ haloalkyl, C₁-C₆ heteroalkyl, C₃-C₈ cycloalkyl, or C₂-C₈ heterocyclyl; and
R^{1A} and R^{1B} are independently selected from hydrogen, halogen, CN, NO₂, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ heteroalkyl, C₁-C₆ haloalkyl, C₃-C₈ cycloalkyl, C₂-C₈ heterocyclyl, aryl or heteroaryl, or
R^{1A} and R^{1B}, together with the atom(s) to which they are connected, optionally form C₃-C₁₃ cycloalkyl, C₂-C₁₂ heterocyclyl, aryl, or heteroaryl.

5. The heterobifunctional compound of claim 4, or a pharmaceutically acceptable salt thereof, wherein X¹ is O or S; and X² is N.

6. The heterobifunctional compound of any one of claims 1-5, or a pharmaceutically acceptable salt thereof, wherein R² is H.

7. The heterobifunctional compound of claim 4, or a pharmaceutically acceptable salt thereof, wherein the DDB1 binding moiety of Formula (II) has the structure of Formula (IV-1):

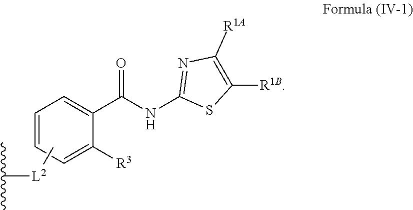

8. The heterobifunctional compound of claim 4, or a pharmaceutically acceptable salt thereof, wherein the DDB1 binding moiety of Formula (II) has the structure of Formula (IV-4): 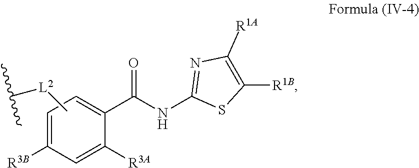
wherein,
R^{3A} and R^{3B} are each independently hydrogen, halogen, —CN, —NO₂, —OR^{4A}, —NR^{4A}R^{4B}, —C(═O)R^{4A}, —C(═O)OR^{4A}, —C(═O)NR^{4B}R^{4A}, —OC(═O)R^{4A}, —N(R^{4A})C(═O)R^{4B}, C₁-C₆ alkyl, C₁-C₆ haloalkyl, C₁-C₆ heteroalkyl, C₃-C₈ cycloalkyl, C₂-C₈ heterocyclyl, aryl, or heteroaryl; and
each R^{4A} and R^{4B} is independently hydrogen, C₁-C₆ alkyl, C₂-C₆ alkenyl, C₂-C₆ alkynyl, C₁-C₆ haloalkyl, C₁-C₆ heteroalkyl, C₃-C₈ cycloalkyl, C₂-C₈ heterocyclyl, aryl, or heteroaryl, or
R^{4A} and R^{4B}, together with the atom(s) to which they are connected, optionally form C₂-C₁₂ heterocyclyl.

9. The heterobifunctional compound of claim 4, or a pharmaceutically acceptable salt thereof, wherein the DDB1 binding moiety of Formula (II) has the structure of Formula (IV-5): 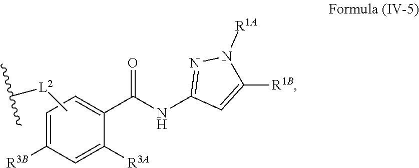
wherein,
R^{3A} and R^{3B} are each independently hydrogen, halogen, —CN, —NO₂, —OR^{4A}, —NR^{4A}R^{4B}, —C(═O)R^{4A}, —C(═O)OR^{4A}, —C(═O)NR^{4B}R^{4A}, —OC(═O)R^{4A}, —N(R^{4A})C(═O)R^{4B}, C₁-C₆ alkyl, C₁-C₆ haloalkyl, C₁-C₆ heteroalkyl, C₃-C₈ cycloalkyl, C₂-C₈ heterocyclyl, aryl, or heteroaryl; and
each R^{4A} and R^{4B} is independently hydrogen, C₁-C₆ alkyl, C₂-C₆ alkenyl, C₂-C₆ alkynyl, C₁-C₆ haloalkyl, C₁-C₆ heteroalkyl, C₃-C₈ cycloalkyl, C₂-C₈ heterocyclyl, aryl, or heteroaryl, or R^{4A} and R^{4B}, together with the atom(s) to which they are connected, optionally form C₂-C₁₂ heterocyclyl.

10. The heterobifunctional compound of any one of claims 4-9, or a pharmaceutically acceptable salt thereof, wherein R^{IA} is selected from hydrogen, halogen, —OCH₃, —NH₂, —NHCH₃, —N(CH₃)₂, —C(═O)CH₃, —C(═O)OCH₃, —C(═O)NH₂, —C(═O)NHCH₃, —C(═O)N(CH₃)₂, —CH₃, —CHF₂, —CF₃, —CH₂CH₃, —CH(CH₃)₂, —C(CH₃)₃, cyclopropyl, cyclobutyl, cyclopentyl, cyclohexyl, or phenyl.

11. The heterobifunctional compound of any one of claims 4-9, or a pharmaceutically acceptable salt thereof, wherein R^{1B} is selected from hydrogen, halogen, —OCH₃, —NH₂, —NHCH₃, —N(CH₃)₂, —C(═O)CH₃, —C(═O)OCH₃, —C(═O)NH₂, —C(═O)NHCH₃, —C(═O)N(CH₃)₂, —CHF₂, —CF₃, or phenyl.

12. The heterobifunctional compound of any one of claims 4-9, or a pharmaceutically acceptable salt thereof, wherein R^{1B} is selected from —CH₃, —CH(CH₃)₂, —C(CH₃)₃, cyclopropyl, cyclobutyl, cyclopentyl, or cyclohexyl.

13. The heterobifunctional compound of claim 1, or a pharmaceutically acceptable salt thereof, wherein ring Q is a phenyl or a 6-membered monocyclic heteroaryl.

14. The heterobifunctional compound of claim 13, or a pharmaceutically acceptable salt thereof, wherein the 6-membered monocyclic heteroaryl is pyridinyl, pyridazinyl, pyrazinyl, pyrimidinyl, or triazynyl.

15. The heterobifunctional compound of claim 13 or 14, or a pharmaceutically acceptable salt thereof, wherein the DDB1 binding moiety of Formula (II) has the structure of Formula (V-1): 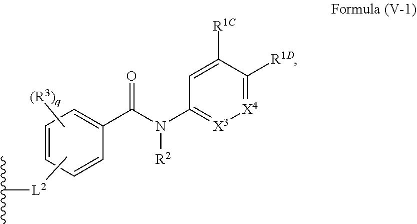
wherein,
X³ is N or CH;
X⁴ is CR^{1E} or N; and
each of R^{1C}, R^{1D}, and R^{1E} is independently selected from hydrogen, halogen, CN, —NO₂, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, C₂-C₈ heterocyclyl, aryl or heteroaryl, or
R^{1C} and R^{1D}, or R^{1D} and R^{1E}, together with the atom(s) to which they are connected, optionally form C₃-C₁₃ cycloalkyl, C₂-C₁₂ heterocyclyl, aryl, or heteroaryl.

16. The heterobifunctional compound of claim 15, or a pharmaceutically acceptable salt thereof, wherein R² is hydrogen.

17. The heterobifunctional compound of claim 15, or a pharmaceutically acceptable salt thereof, wherein the DDB1 binding moiety of Formula (II) has the structure of Formula (V-2): 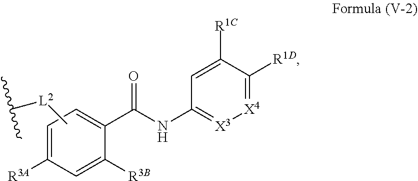
wherein,
R^{3A} and R^{3B} are each independently hydrogen, halogen, —CN, —NO₂, —OR^{4A}, —NR^{4A}R^{4B}, —C(═O)R^{4A}, —C(═O)OR^{4A}, —C(═O)NR^{4B}R^{4A}, —OC(═O)R^{4A}, —N(R^{4A})C(═O)R^{4B}, C₁-C₆ alkyl, C₁-C₆ haloalkyl, C₁-C₆ heteroalkyl, C₃-C₈ cycloalkyl, C₂-C₈ heterocyclyl, aryl, or heteroaryl; and
each R^{4A} and R^{4B} is independently hydrogen, C₁-C₆ alkyl, C₂-C₆ alkenyl, C₂-C₆ alkynyl, C₁-C₆ haloalkyl, C₁-C₆ heteroalkyl, C₃-C₈ cycloalkyl, C₂-C₈ heterocyclyl, aryl, or heteroaryl, or
R^{4A} and R^{4B}, together with the atom(s) to which they are connected, optionally form C₂-C₁₂ heterocyclyl.

18. The heterobifunctional compound of any one of claims 15-17, or a pharmaceutically acceptable salt thereof, wherein X³ is N.

19. The heterobifunctional compound of claim 18, or a pharmaceutically acceptable salt thereof, wherein X⁴ is N.

20. The heterobifunctional compound of claim 18, or a pharmaceutically acceptable salt thereof, wherein X⁴ is CR^{1E}.

21. The heterobifunctional compound of any one of claims 15-17, or a pharmaceutically acceptable salt thereof, wherein X³ is CH.

22. The heterobifunctional compound of any one of claims 15-17, or a pharmaceutically acceptable salt thereof, wherein X⁴ is N.

23. The heterobifunctional compound of any one of claims 15-22, or a pharmaceutically acceptable salt thereof, wherein R^{1C} and R^{1E} are each hydrogen; and R^{1D} is hydrogen, halogen, —CN, —OR^{4A}, —NR^{4B}R^{4A}, —C(═O)R^{4A}, —C(═O)OR^{4A}, —C(═O)NR^{4B}R^{4A}, C₁-C₆ alkyl, C₁-C₆ haloalkyl, C₁-C₆ heteroalkyl, C₃-C₈ cycloalkyl, 4 to 7-membered heterocycloalkyl, aryl, or heteroaryl.

24. The heterobifunctional compound of claim 23, or a pharmaceutically acceptable salt thereof, wherein R^{1D} is C₁-C₆ alkyl, C₁-C₆ heteroalkyl, C₃-C₈ cycloalkyl, or 4 to 7-membered heterocycloalkyl.

25. The heterobifunctional compound of claim 23, or a pharmaceutically acceptable salt thereof, wherein R^{1D} is hydrogen, —NR^{4B}R^{4A}, or OR^{4A}.

26. The heterobifunctional compound of any one of claims 1-25, or a pharmaceutically acceptable salt thereof, wherein each R³ is independently hydrogen, halogen, C₁-C₆ alkyl, C₁-C₆ haloalkyl, C₁-C₆ heteroalkyl, C₁-C₆ alkoxy, C₁-C₆ alkylamino, C₁-C₆ cycloalkoxy, C₁-C₆ cycloalkylamino, C₃-C₈ cycloalkyl, or C₂-C₈ heterocyclyl.

27. The heterobifunctional compound of claim 26, or a pharmaceutically acceptable salt thereof, wherein R³ is F, Cl, Br, CH₃, CHF₂, CF₃, CH₂CH₃, CH(CH₃)₂, cyclopropyl, CN, —NH₂, NH(CH₃), NH(i-Pr), NH(n-Bu), NH(t-Bu), or N(CH₃)₂.

28. The heterobifunctional compound of any one of claims 1-27, or a pharmaceutically acceptable salt thereof, wherein p is 1 or 2.

29. The heterobifunctional compound of any one of claims 1-28, or a pharmaceutically acceptable salt thereof, wherein L² is —C(═O)NR^{4B}—, —C₁-C₃alkylene-, —C₂-C₃alkynylene-, —NR^{4A}—(C₁-C₃alkylene)-, —NR^{4A}—(C₁-C₃alkylene)-C(═O)NR^{4B}, —O—(C₁-C₃ alkylene)-, or —O—(C₁-C₃ alkylene)-C(═O)NR^{4B}—.

30. The heterobifunctional compound of claim 29, or a pharmaceutically acceptable salt thereof, wherein L² is —C(═O)NH—, —CH₂—, —C≡C—, —NH—(CH₂)—, —NH—(CH₂)—C(═O)NH, —O—(CH₂)—, or —O—(CH₂)—C(═O)NH—.

31. The heterobifunctional compound of any one of claims 1-28, or a pharmaceutically acceptable salt thereof, wherein L² is —NR^{4A} or —O—.

32. The heterobifunctional compound of any one of claims 1-28, or a pharmaceutically acceptable salt thereof, wherein L² is —NH—.

33. The heterobifunctional compound of any one of claims 1-28, or a pharmaceutically acceptable salt thereof, wherein L² is —O—.

34. The heterobifunctional compound of any one of claims 1-33, wherein linker L¹ is a divalent moiety having the structure of Formula (L), or a pharmaceutically acceptable salt thereof: 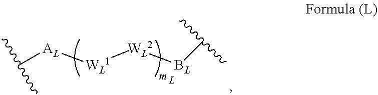
wherein,
A_{L}, W_{L}¹, W_{L}², and B_{L}, at each occurrence, is a bivalent moiety independently selected from the group consisting of a bond, R_{L}^{a}—R_{L}^{b}, R_{L}^{a}COR_{L}^{b}, R_{L}^{a}C(O)OR_{L}^{b}, R_{L}^{a}C(O)N(R_{L})R_{L}^{b}, R_{L}^{a}C(S)N(R_{L}¹)R_{L}^{b}, R_{L}^{a}OR_{L}^{b}, R_{L}^{a}SR_{L}^{b}, R_{L}^{a}SOR_{L}^{b}, R_{L}^{a}SO₂R_{L}^{b}, R_{L}^{a}SO₂N(R_{L}¹)R_{L}^{b}, R_{L}^{a}N(R_{L}¹)R_{L}^{b}, R_{L}^{a}N(R_{L}¹)COR_{L}^{b}, R_{L}^{a}N(R_{L}¹)CON(R_{L}²)R_{L}^{b}, R_{L}^{a}N(R_{L}¹)C(S)R_{L}^{b}, optionally substituted C₁-C₈ alkylene, optionally substituted C₂-C₈ alkenylene, optionally substituted C₂-C₈ alkynylene, optionally substituted 1-8 membered heteroalkylene, optionally substituted 2-8 membered heteroalkenylene, optionally substituted 2-8 membered heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ cycloalkylene, optionally substituted 3-13 membered heterocyclene, optionally substituted arylene, and optionally substituted heteroarylene, wherein
each R_{L}^{a} and R_{L}^{b} is independently a bond, R_{L}^{r}, optionally substituted (C₁-C₈ alkylene)-R_{L}^{r}, optionally substituted R_{L}^{r}—(C₁-C₈ alkylene), optionally substituted (C₁-C₈ alkylene)-R_{L}^{r}—(C₁-C₈ alkylene), or a bivalent moiety comprising of optionally substituted C₁-C₈ alkylene, optionally substituted C₂-C₈ alkenylene, optionally substituted C₂-C₈ alkynylene, optionally substituted 1-8 membered heteroalkylene, optionally substituted 2-8 membered heteroalkenylene, optionally substituted 2-8 membered heteroalkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ cycloalkylene, optionally substituted 3-13 membered heterocyclene, optionally substituted arylene, or optionally substituted heteroarylene;
each R_{L}^{r} is independently selected from optionally substituted C₃-C₁₀ cycloalkylene, optionally substituted 3-10 membered heterocyclene, optionally substituted arylene, and optionally substituted heteroarylene;
each R_{L}¹ and R_{L}² are independently selected from the group consisting of hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ cycloalkyl, optionally substituted 3-10 membered heterocyclyl, optionally substituted aryl, and optionally substituted heteroaryl; or
R_{L}^{a} and R_{L}^{b}, R_{L}¹ and R_{L}², R_{L}^{a} and R_{L}¹, R_{L}^{a} and R_{L}², R_{L}^{b} and R_{L}¹, or R_{L}^{b} and R_{L}² together with the atom(s) to which they are attached optionally form a C₃-C₂₀ carbocyclyl or 3-20 membered heterocyclyl ring; and
m_{L} is an integer selected from 1 to 15.

35. The heterobifunctional compound of claim 34, or a pharmaceutically acceptable salt thereof, wherein A_{L} is a bond, —C(═O)—, —C(═O)NH—, —NH—, —NH—C(═O)—, —O—, —(C₁-C₈ alkylene)-C(═O)NH—, —(C₁-C₈ alkylene)-C(═O)—, —(C₁-C₈ alkylene)NH—, —(C₁-C₈ alkylene)-NH—C(═O)—, —(C₁-C₈ alkylene)-O—, —C₁-C₈ alkylene-, or —C₂-C₈ alkynylene.

36. The heterobifunctional compound of claim 34 or 35, or a pharmaceutically acceptable salt thereof, wherein B_{L} is a bond, —C(═O)—, —C(═O)NH—, —NH—, —NH—C(═O)—, —O—, —(C₁-C₈ alkylene)-, —C₂-C₈ alkynylene-, —NH—(C₁-C₈ alkylene)-, —O—(C₁-C₈ alkylene)-, —C(═O)—(C₁-C₈ alkylene)-, —C(═O)NH—(C₁-C₈ alkylene)-, or —NH—C(═O)—(C₁-C₈ alkylene)-.

37. The heterobifunctional compound of any one of claims 34-36, or a pharmaceutically acceptable salt thereof, wherein each W_{L}¹ is independently R_{L}^{r} or C₁, —C₃ alkylene; and each W_{L}² is independently a bond, O, or NH.

38. The heterobifunctional compound of any one of claims 34-36, or a pharmaceutically acceptable salt thereof, wherein each W_{L}¹ is independently a bond, O, or NH; and each W_{L}² is independently R_{L}^{r}, or C₁-C₃ alkylene.

39. The heterobifunctional compound of any one of claims 34-36, or a pharmaceutically acceptable salt thereof, wherein each W_{L}¹ is independently C₁-C₃ alkylene; and each W_{L}² is independently a bond or O.

40. The heterobifunctional compound of any one of claims 34-36, or a pharmaceutically acceptable salt thereof, wherein each W_{L}¹ is independently a bond or O; and each W_{L}² is independently C₁-C₃ alkylene.

41. The heterobifunctional compound of any one of claims 34-36, or a pharmaceutically acceptable salt thereof, wherein each —W_{L}¹—W_{L}²— is independently —CH₂CH₂O— or —CH₂—.

42. The heterobifunctional compound of any one of claims 34-41, or a pharmaceutically acceptable salt thereof, wherein m_{L} is an integer selected from 1 to 10.

43. The heterobifunctional compound of any one of claims 1-33, or a pharmaceutically acceptable salt thereof, wherein linker L¹ is —(CH₂)ₚ₁C(═O)NH(CH₂CH₂O)ₚ₂—(CH₂)ₚ₃—, —(CH₂)ₚ₁C(═O)NH(CH₂)ₚ₂—, —(CH₂)ₚ₁NHC(═O)—(CH₂CH₂O)ₚ₂—(CH₂)ₚ₃—, —(CH₂)ₚ₁NHC(═O)—(CH₂)ₚ₂—, —(CH₂)ₚ₁C(═O)—(CH₂CH₂O)ₚ₂—(CH₂)ₚ₃—, —(CH₂)ₚ₁C(═O)—(CH₂)ₚ₂—, —(CH₂)ₚ₁NH(CH₂CH₂O)ₚ₂—(CH₂)ₚ₃—, —(CH₂)ₚ₁NH(CH₂)ₚ₂—, —(CH₂CH₂O)ₚ₂—(CH₂)ₚ₃—, or —(CH₂)ₚ₂—; wherein p1 is an integer selected from 0 to 8; p2 is an integer selected from 1 to 15; and p3 is an integer selected from 0 to 8.

44. The heterobifunctional compound of any one of claims 1-43, or a pharmaceutically acceptable salt thereof, wherein A is a target protein binding moiety comprising a cyclin-dependent kinase 4 (CDK4) binding moiety and/or a cyclin-dependent kinase 6 (CDK6) binding moiety.

45. The heterobifunctional compound of any one of claims 1-44, wherein A is a target protein binding moiety of Formula (A), or a pharmaceutically acceptable salt thereof: 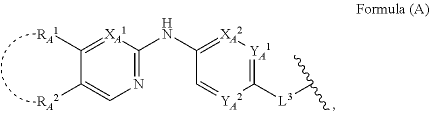
wherein,
X_{A}¹, X_{A}², Y_{A}¹, and Y_{A}² are each independently CR_{A}⁴ or N;
R_{A}¹ is NR_{A}⁵R_{A}⁶, N(R_{A}⁵)C(═O)R_{A}⁶, aryl, or heteroaryl;
R_{A}² is hydrogen, halogen, CN, NO₂, C₁-C₈ alkyl, C₁-C₈ haloalkyl, C₁-C₈ alkoxy, C₁-C₈ heteroalkyl, C₃-C₈ cycloalkyl, or C₂-C₈ heterocyclyl, or
R_{A}¹ and R_{A}², together with the atom(s) to which they are attached optionally form an optionally substituted cycloalkyl, heterocyclyl, aryl or heteroaryl;
L³ is a divalent group selected from —R_{A}^{3A}—R_{A}^{3B}—, wherein R_{A}^{3A} and R_{A}^{3B} are each independently a bond, —O—, —S—, —NR_{A}⁷—, —C(═O)—, —C(═O)NR_{A}⁷—, —S(═O)—, —S(═O)NR_{A}⁷—, —S(═O)₂—, —S(═O)₂NR_{A}⁷—, C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₃ cycloalkylene, C₂-C₁₂ heterocyclene, arylene, or heteroarylene;
each R_{A}⁴ is independently selected from hydrogen, halogen, CN, NO₂, NR_{A}⁸R_{A}⁹, —C(═O)R_{A}¹⁰, —C(═O)OR_{A}¹⁰, —C(═O)NR_{A}⁸R_{A}⁹, —NR_{A}⁸C(═O)R_{A}¹⁰, C₁-C₈ alkyl, C₁-C₈ haloalkyl, C₁-C₈ alkoxy, C₁-C₈ alkoxyalkyl, C₁-C₈ heteroalkyl, C₃-C₈ cycloalkyl, C₂-C₈ heterocyclyl, aryl, or heteroaryl;
R_{A}⁵ and R_{A}⁶ are independently selected from hydrogen, C₁-C₈ alkyl, C₁-C₈ haloalkyl, C₁-C₈ alkoxyalkyl, C₁-C₈ heteroalkyl, C₃-C₈ cycloalkyl, C₂-C₈ heterocyclyl, aryl, or heteroaryl, or R_{A}⁵ and R_{A}⁶ together with the atom(s) to which they are connected optionally form a 3-20 membered heterocyclyl ring; and
R_{A}⁷, R_{A}⁸, R_{A}⁹ and R_{A}¹⁰ are each independently selected from hydrogen, C₁-C₈ alkyl, C₁-C₈ haloalkyl, C₁-C₈ alkoxyalkyl, C₁-C₈ heteroalkyl, C₃-C₈ cycloalkyl, C₂-C₈ heterocyclyl, aryl, or heteroaryl, or
R_{A}⁸ and R_{A}⁹ together with the atom(s) to which they are connected optionally form a 3-20 membered heterocyclyl ring.

46. The heterobifunctional compound of claim 45, or a pharmaceutically acceptable salt thereof, wherein R_{A}¹ and R_{A}², together with the atom(s) to which they are connected, form an optionally substituted heterocyclyl or heteroaryl.

47. The heterobifunctional compound of claim 45 or 46, wherein the target protein binding moiety of Formula (A) has the structure of Formula (A1), (A2), or (A3), or a pharmaceutically acceptable salt thereof: 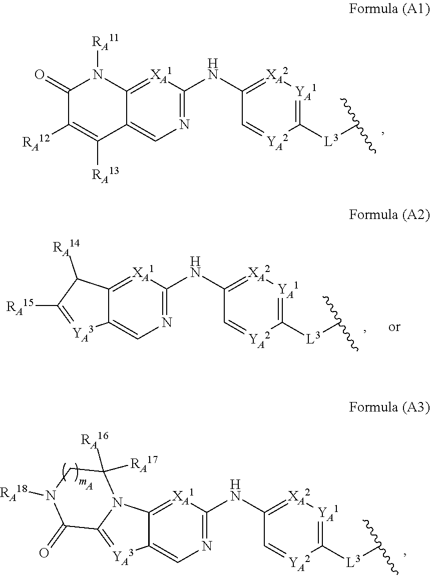
wherein
Y_{A}³ is CR_{A}¹⁹ or N;
R_{A}¹¹, R_{A}¹⁴ and R_{A}¹⁸ are each independently selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, or C₂-C₈ heterocyclyl, aryl, or heteroaryl;
R_{A}¹² and R_{A}¹⁵ are each independently selected from R_{A}²⁰, COR_{A}²⁰, CO₂R_{A}²⁰, or CONR_{A}²⁰R_{A}²¹ wherein R_{A}²⁰ and R_{A}²¹ are independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or C₂-C₈ heterocyclyl, or R_{A}²⁰ and R_{A}²¹, together with the atom(s) to which they are connected optionally form a 3-20 membered heterocyclyl ring;
R_{A}¹³ is selected from hydrogen, halogen, C₁-C₈ alkyl, C₁-C₈ haloalkyl, C₁-C₈ alkoxy, C₁-C₈ alkylamino, C₁-C₈ heteroalkyl, C₃-C₈ cycloalkyl, or C₂-C₈ heterocyclyl;
R_{A}¹⁶ and R_{A}¹⁷ are each independently selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, or C₂-C₈ heterocyclyl, aryl, or heteroaryl, or
R_{A}¹⁶ and R_{A}¹⁷, together with the atom(s) to which they are connected optionally form 3-8 membered cycloalkyl, or 3-8 membered heterocyclyl;
R_{A}¹⁹ are independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or C₂-C₈ heterocyclyl; and
m_{A} is 0, 1, or 2.

48. The heterobifunctional compound of claim 47, or a pharmaceutically acceptable salt thereof, wherein m_{A} is 1.

49. The heterobifunctional compound of claim 45, or a pharmaceutically acceptable salt thereof, wherein R_{A}¹ is aryl or heteroaryl.

50. The heterobifunctional compound of claim 45 or 49, wherein the target protein binding moiety of Formula (A) has the structure of Formula (A4), or a pharmaceutically acceptable salt thereof: 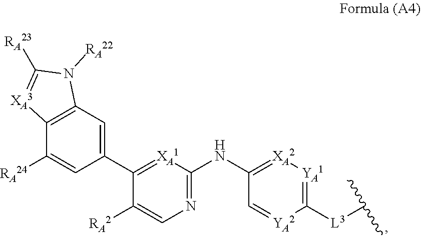
wherein
X_{A}³ is CR_{A}²⁵ or N;
R_{A}²² is selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, C₂-C₈ heterocyclyl, aryl, or heteroaryl; and
R_{A}²³, R_{A}²⁴ and R_{A}²⁵ are each independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or C₂-C₈ heterocyclyl.

51. The heterobifunctional compound of any one of claims 45-50, or a pharmaceutically acceptable salt thereof, wherein X_{A}¹, X_{A}², and X_{A}³ are each N.

52. The heterobifunctional compound of any one of claims 45-50, or a pharmaceutically acceptable salt thereof, wherein Y_{A}¹, Y_{A}², and Y_{A}³ are each CH.

53. The heterobifunctional compound of any one of claims 45-50, or a pharmaceutically acceptable salt thereof, wherein R_{A}², R_{A}⁴, R_{A}¹³, R_{A}¹⁹, R_{A}²³, and R_{A}²⁴ are each independently selected from hydrogen, halogen, C₁-C₃ alkyl, or C₃-C₆ cycloalkyl.

54. The heterobifunctional compound of claim 53, or a pharmaceutically acceptable salt thereof, wherein R_{A}², R_{A}⁴, R_{A}¹³, R_{A}¹⁹, R_{A}²³, and R_{A}²⁴ are each independently selected from hydrogen, F, Cl, CH₃, CH₂CH₃, CH(CH₃)₂, CF₃, CHF₂, cyclopropyl, or cyclobutyl.

55. The heterobifunctional compound of any one of claims 47-54, or a pharmaceutically acceptable salt thereof, wherein R_{A}¹¹ and R_{A}¹⁴ are each independently selected from hydrogen, C₁-C₈ alkyl, C₃-C₈ cycloalkyl, or C₂-C₈ heterocyclyl.

56. The heterobifunctional compound of claim 55, or a pharmaceutically acceptable salt thereof, wherein R_{A}¹¹ and R_{A}¹⁴ are each independently selected from C₁-C₈ alkyl, or C₃-C₈ cycloalkyl.

57. The heterobifunctional compound of any one of claims 47-56, or a pharmaceutically acceptable salt thereof, wherein R_{A}¹² and R_{A}¹⁵ are each independently selected from R_{A}²⁰, COR_{A}²⁰, or CONR_{A}²⁰R_{A}²¹, wherein R_{A}²⁰ and R_{A}²¹ are each independently selected from C₁-C₈ alkyl, C₃-C₈ cycloalkyl, or C₂-C₈ heterocyclyl.

58. The heterobifunctional compound of claim 57, or a pharmaceutically acceptable salt thereof, wherein R_{A}¹² and R_{A}¹⁵ are each independently selected from COR_{A}²⁰, or CONR_{A}²⁰R_{A}²¹, wherein R_{A}²⁰ and R_{A}²¹ are each independently selected from C₁-C₈ alkyl.

59. The heterobifunctional compound of any one of claims 47-58, wherein R_{A}¹⁶ and R_{A}¹⁷ are each independently selected from hydrogen, C₁-C₈ alkyl, C₃-C₈ cycloalkyl, or C₂-C₈ heterocyclyl.

60. The heterobifunctional compound of any one of claims 47-58, or a pharmaceutically acceptable salt thereof, wherein R_{A}¹⁶ and R_{A}²² together with the atom(s) to which they are connected optionally form a 3-6 membered cycloalkyl or 3-6 membered heterocyclyl ring.

61. The heterobifunctional compound of any one of claims 47-60, or a pharmaceutically acceptable salt thereof, wherein R_{A}¹⁸ and R_{A}²² are each independently selected from hydrogen, C₁-C₈ alkyl, C₃-C₈ cycloalkyl, or C₂-C₈ heterocyclyl.

62. The heterobifunctional compound of claim 61, or a pharmaceutically acceptable salt thereof, wherein R_{A}¹⁸ and R_{A}²² are each independently selected from H, CH₃, CH₂CH₃, CH(CH₃)₂, CF₃, CHF₂, cyclopropyl, or cyclobutyl.

63. The heterobifunctional compound of any one of claims 45-62, or a pharmaceutically acceptable salt thereof, wherein L³ is a bond, C₁-C₃ alkylene, C₃-C₈ cycloalkylene, C₂-C₈ heteroalkylene, C₂-C₈ heterocyclyl, —(C₁-C₃ alkylene)-(C₃-C₈ cycloalkylene)-, —(C₁-C₃ alkylene)-(C₂-C₈ heterocyclyl)-, or —(C₁-C₃ alkylene)-(C₂-C₈ heteroalkylene)-.

64. The heterobifunctional compound of any one of claims 45-63, or a pharmaceutically acceptable salt thereof, wherein L³ is a bond,

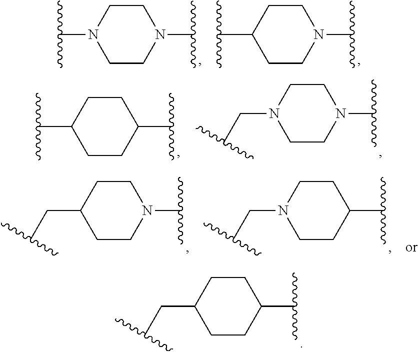

65. The heterobifunctional compound of any one of claims 45-64, wherein the target protein binding moiety is selected from: 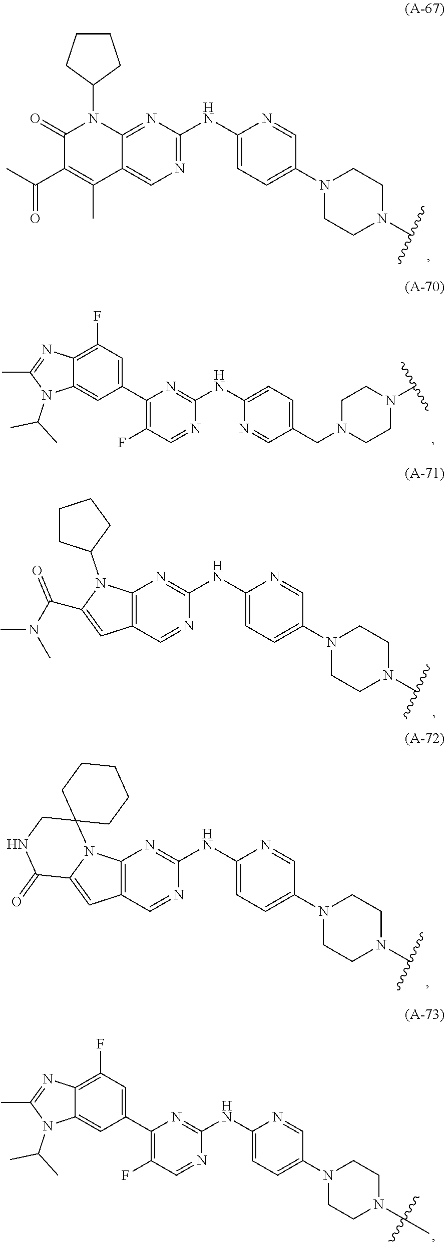 [SMILES: *N1CCN(c2ccc(Nc3ncc4c(C)c(C(C)=O)c(=O)n(C5CCCC5)c4n3)nc2)CC1 ; *N1CCN(Cc2ccc(Nc3ncc(F)c(-c4cc(F)c5nc(C)n(C(C)C)c5c4)n3)nc2)CC1 ; *N1CCN(c2ccc(Nc3ncc4cc(C(=O)N(C)C)n(C5CCCC5)c4n3)nc2)CC1 ; *N1CCN(c2ccc(Nc3ncc4cc5n(c4n3)C3(CCCCC3)CNC5=O)nc2)CC1 ; *N1CCN(c2ccc(Nc3ncc(F)c(-c4cc(F)c5nc(C)n(C(C)C)c5c4)n3)nc2)CC1]
or a pharmaceutically acceptable salt thereof.

66. The heterobifunctional compound of any one of claims 1-43, or a pharmaceutically acceptable salt thereof, wherein A is a target protein binding moiety comprising a CBP and/or p300 binding moiety.

67. The heterobifunctional compound of any one of claims 1-43, wherein A is a target protein binding moiety having the structure of Formula (B-1), or a pharmaceutically acceptable salt thereof: 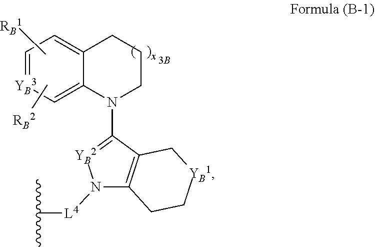
wherein,
Y_{B}¹ is CHR_{B}⁴ or NR_{B}⁴;
Y_{B}² is CH or N;
Y_{B}³ is CR_{B}² or N;
R_{B}¹ is an optionally substituted 5-6 membered heteroaryl;
each R_{B}² is independently hydrogen, halogen, CN, NO₂, C₁-C₈ alkyl, C₁-C₈ haloalkyl, C₁-C₈ alkoxy, C₁-C₈ heteroalkyl, C₃-C₈ cycloalkyl, or C₂-C₈ heterocyclyl;
R_{B}⁴ is —C(═O)R_{B}⁸, —C(═O)OR_{B}⁸, —C(═O)NR_{B}⁶R_{B}⁷, or —NR_{B}⁶C(═O)R_{B}⁸;
L⁴ is a divalent group selected from —R_{B}^{3A}—R_{B}^{3B}—, wherein
R_{B}^{3A} and R_{B}^{3B} are each independently a bond, —O—, —S—, —NR_{B}⁵—, —C(═O)—, —C(═O)NR_{B}⁵—, —S(═O)—, —S(═O)NR_{B}⁵—, —S(═O)₂—, —S(═O)₂NR_{B}⁵—, C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₃ cycloalkylene, C₂-C₁₂ heterocyclene, arylene, or heteroarylene;
R_{B}⁵, R_{B}⁶, R_{B}⁷ and R_{B}⁸ are each independently selected from C₁-C₈ alkyl, C₁-C₈ haloalkyl, C₁-C₈ alkoxyalkyl, C₁-C₈ heteroalkyl, C₃-C₈ cycloalkyl, C₂-C₈ heterocyclyl, aryl, or heteroaryl, or
R_{B}⁶ and R_{B}⁷ together with the atom(s) to which they are connected optionally form a 3-20 membered heterocyclyl ring; and
x_{3B} is 0, 1, or 2.

68. The heterobifunctional compound of claim 67, or a pharmaceutically acceptable salt thereof, wherein Y_{B}² is N; and x_{3B} is 1.

69. The heterobifunctional compound of claim 67 or 68, or a pharmaceutically acceptable salt thereof, wherein Y_{B}¹ is NR_{B}⁴.

70. The heterobifunctional compound of claim 67 or 68, or a pharmaceutically acceptable salt thereof, wherein Y_{B}³ is CR_{B}².

71. The heterobifunctional compound of any one of claims 67-70, wherein A is a target protein binding moiety having the structure of Formula (B-2), or a pharmaceutically acceptable salt thereof:

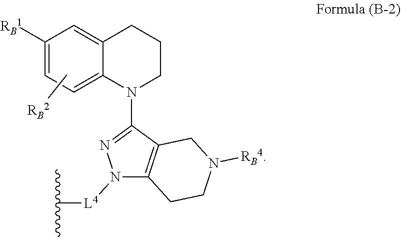

72. The heterobifunctional compound of any one of claim 67-71, or a pharmaceutically acceptable salt thereof, wherein R_{B}⁴ is —C(═O)R_{B}⁸ or —C(═O)NHR_{B}⁸, wherein R_{B}⁸ is C₁-C₈ alkyl.

73. The heterobifunctional compound of any one of claims 67-72, or a pharmaceutically acceptable salt thereof, wherein R_{B}² is halogen, CN, NO₂, C₁-C₈ alkyl, C₁-C₈ haloalkyl, or C₁-C₈ alkoxy.

74. The heterobifunctional compound of claim 67-73, or a pharmaceutically acceptable salt thereof, wherein R_{B}¹ is an optionally substituted 5-membered heteroaryl selected from pyrrolyl, furanyl, imidazolyl, pyrazolyl, oxazolyl, isoxazolyl, thienyl, thiazolyl, isothiazolyl, triazolyl, oxadiazolyl, thiadiazolyl, or tetrazolyl.

75. The heterobifunctional compound of any one of claims 67-74, or a pharmaceutically acceptable salt thereof, wherein L⁴ is a bond, C₁-C₃ alkylene, C₃-C₈ cycloalkylene, C₂-C₈ heteroalkylene, C₂-C₈ heterocyclene, —(C₁-C₃ alkylene)-(C₃-C₈ cycloalkylene)-, —(C₁-C₃ alkylene)-(C₂-C₈ heterocyclene)-, or —(C₁-C₃ alkylene)-(C₂-C₈ heteroalkylene)-.

76. The heterobifunctional compound of any one of claims 67-75, or a pharmaceutically acceptable salt thereof, wherein L⁴ is a bond,

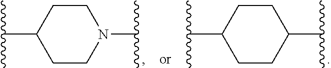 [SMILES: *C1CCN(*)CC1 ; *C1CCC(*)CC1]

77. The heterobifunctional compound of any one of claims 67-76, wherein the target protein binding moiety is: 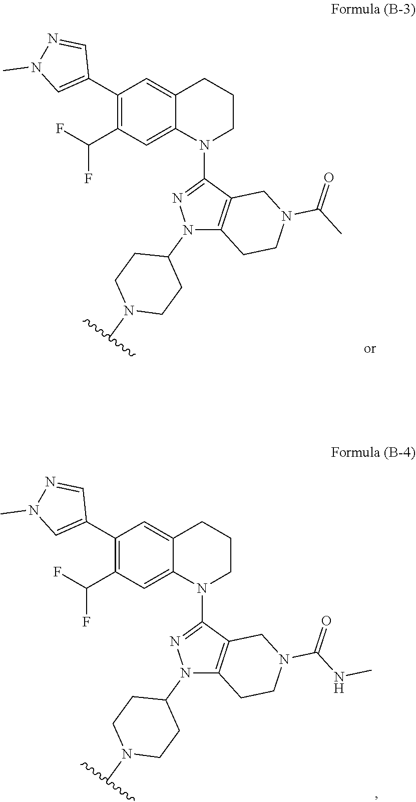
or a pharmaceutically acceptable salt thereof.

78. The heterobifunctional compound of any one of claims 1-43, wherein A is a target protein binding moiety having the structure of Formula (C-1), (C-2), (C-3), (C-4), (C-5), or (C-6), or a pharmaceutically acceptable salt thereof: 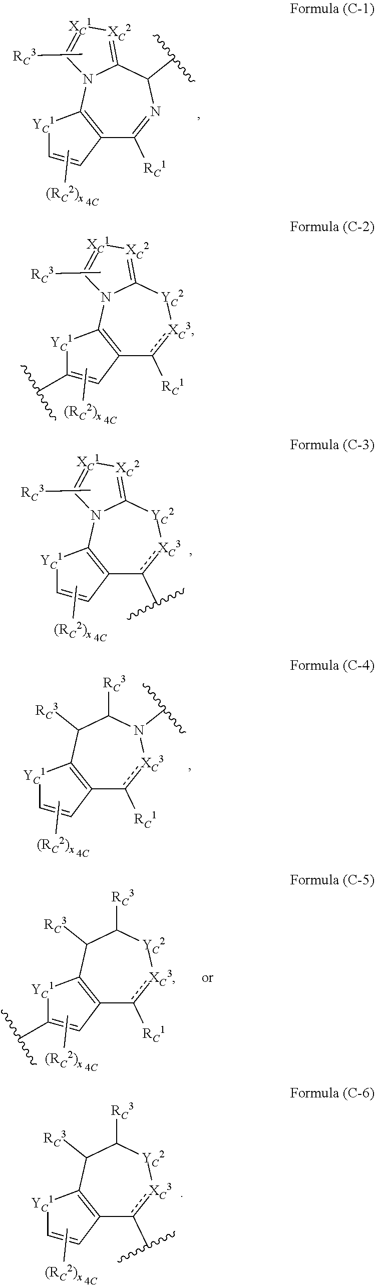
wherein, 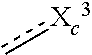
is 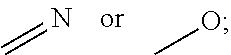
X_{C}¹ and X_{C}² are each independently CR_{C}³ or N;
Y_{C}¹ is O, S, or —C(R_{C}²)═C(R_{C}²)—;
Y_{C}² is C(R_{C}⁷)₂, or NR_{C}⁷;
R_{C}¹ is hydrogen or optionally substituted C₆-C₁₀ aryl or 5 to 10 membered heteroaryl;
each R_{C}² is independently hydrogen, halogen, CN, NO₂, NR_{C}⁴R_{C}⁵, —C(═O)R_{C}⁶, —C(═O)OR_{C}⁴, —C(═O)NR_{C}⁴R_{C}⁵, —OC(═O)R_{C}⁶, —N(R_{C}⁴)C(═O)R_{C}⁶, C₁-C₈ alkyl, C₁-C₈ heteroalkyl, C₂-C₈ alkynyl, C₁-C₈ haloalkyl, C₁-C₈ alkoxy, C₁-C₈ alkoxyalkyl, or C₁-C₈ alkylaryl;
each R_{C}³ is independently hydrogen, halogen, CN, NO₂, NR_{C}⁴R_{C}⁵, C₁-C₈ alkyl, C₁-C₈ haloalkyl, C₁-C₈ alkoxy, C₁-C₈ alkoxyalkyl, aryl, or heteroaryl;
R_{C}⁴, R_{C}⁵ and R_{C}⁶ are each independently selected from hydrogen, C₁-C₈ alkyl, C₁-C₈ haloalkyl, C₁-C₈ alkoxyalkyl, C₁-C₈ heteroalkyl, C₃-C₈ cycloalkyl, C₂-C₈ heterocyclyl, aryl, or heteroaryl, or
R_{C}⁴ and R_{C}⁵ together with the atom(s) to which they are connected optionally form a 3-20 membered heterocyclyl ring;
each R_{C}⁷ is independently hydrogen, NR_{C}⁴R_{C}⁵, OR_{C}⁴, —C(═O)R_{C}⁶, —C(═O)OR_{C}⁶, —C(═O)NR_{C}⁴R_{C}⁵, —(C₁-C₈ alkyl)—C(═O)NR_{C}⁴R_{C}⁵, —OC(═O)R_{C}⁶, —N(R_{C}⁸)C(═O)R_{C}⁶, C₁-C₈ alkyl, C₁-C₈ haloalkyl, C₁-C₈ heteroalkyl, C₃-C₈ cycloalkyl, C₂-C₈ heterocyclyl, or
two of R_{C}⁷, together with the atom(s) they are connected, optionally form a C₃-C₈ cycloalkyl, or C₂-C₈ heterocyclyl; and
x_{4C} is 1, 2, or 3.

79. The heterobifunctional compound of claim 78, or a pharmaceutically acceptable salt thereof, wherein X_{C}¹ and X_{C}² are each independently N.

80. The heterobifunctional compound of claim 78 or 79, or a pharmaceutically acceptable salt thereof, wherein Y_{C}¹ is S.

81. The heterobifunctional compound of any one of claims 78-80, or a pharmaceutically acceptable salt thereof, wherein R_{C}³ is hydrogen, halogen, C₁-C₈ alkyl, C₁-C₈ haloalkyl, C₁-C₈ alkoxy, or C₁-C₈ alkoxyalkyl.

82. The heterobifunctional compound of any one of claims 78-81, or a pharmaceutically acceptable salt thereof, wherein each R_{C}² is independently hydrogen, halogen, C₁-C₈ alkyl, C₂-C₈ alkynyl, C₁-C₈ haloalkyl, C₁-C₈ alkoxy, C₁-C₈ alkoxyalkyl, aryl, or heteroaryl.

83. The heterobifunctional compound of any one of claims 78-82, or a pharmaceutically acceptable salt thereof, wherein x_{4C} is 2; and each R_{C}² is independently, C₁-C₈ alkyl or C₁-C₈ alkoxy.

84. The heterobifunctional compound of any one of claims 78-83, or a pharmaceutically acceptable salt thereof, wherein R_{C}¹ is optionally substituted C₆-C₁₀ aryl, optionally substituted with 1-4 halogen, CN, NO₂, NR_{C}⁴R_{C}⁵, —C(═O)R_{C}⁶, —C(═O)OR_{C}⁶, —C(═O)NR_{C}⁴R_{C}⁵, C₁-C₈ alkyl, C₁-C₈ haloalkyl, C₁-C₈ alkoxy, or C₁-C₈ alkoxyalkyl.

85. The heterobifunctional compound of claim 84 or a pharmaceutically acceptable salt thereof, wherein R_{C}¹ is optionally substituted C₆ aryl, optionally substituted with 1-4 halogen, CN, NO₂, NR_{C}⁴R_{C}⁵, C₁-C₈ alkyl, C₁-C₈ haloalkyl, C₁-C₈ alkoxy, or C₁-C₈ alkoxyalkyl.

86. The heterobifunctional compound of any one of claims 78-85, wherein the target protein binding moiety is: 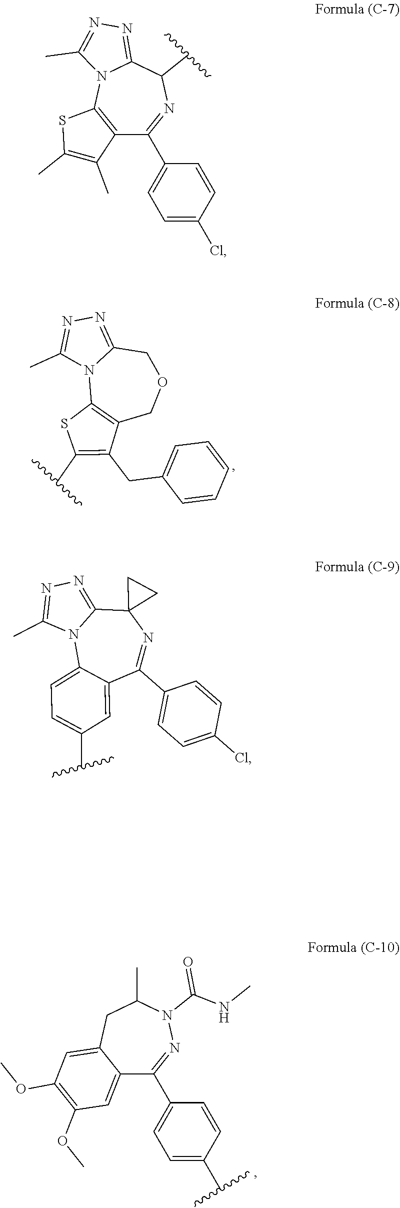
or a pharmaceutically acceptable thereof.

87. The heterobifunctional compound of any one of claim 1-86, wherein the compound is a compound of Table 4, or a pharmaceutically acceptable salt thereof.

88. The heterobifunctional compound of any one of claims 1-87, or a pharmaceutically acceptable salt thereof, wherein the DDB1 binding moiety binds to a binding region on the DDB1 protein, wherein the binding region comprises a beta propeller domain.

89. The heterobifunctional compound of claim 88, or a pharmaceutically acceptable salt thereof, wherein the beta propeller domain comprises a beta propeller C (BPC) domain.

90. The heterobifunctional compound of claim 88 or 89, or a pharmaceutically acceptable salt thereof, wherein the binding region comprises one or more of the following DDB1 residues: ARG327, LEU328, PRO358, ILE359, VAL360, ASP361, GLY380, ALA381, PHE382, SER720, ARG722, LYS723, SER738, ILE740, GLU787, TYR812, LEU814, SER815, ALA834, VAL836, ALA841, ALA869, TYR871, SER872, MET910, LEU912, TYR913, LEU926, TRP953, SER955, ALA956, ASN970, ALA971, PHE972, PHE1003, ASN1005, VAL1006, or VAL1033.

91. An in vivo modified protein comprising a DNA damage-binding protein 1 (DDB1) protein directly bound to a DDB1 ligand, wherein the DDB1 ligand comprises the heterobifunctional compound of any one of claims 1-86, or a pharmaceutically acceptable salt thereof.

92. A method of degrading a target protein, comprising contacting the target protein with the heterobifunctional compound of any one of claims 1-86, or a pharmaceutically acceptable salt thereof.

93. A method for the treatment of cancer in a subject in need thereof, comprising administering to the subject a therapeutically effective amount of the heterobifunctional compound of any one of claims 1-86, or a pharmaceutically acceptable salt thereof.

94. A method for the treatment of cancer in a subject in need thereof, comprising administering to the subject a therapeutically effective amount of the heterobifunctional compound of any one of claims 44-65, or a pharmaceutically acceptable salt thereof.

95. The method of claim 93 or 94, wherein the cancer is selected from the group consisting of breast cancer, ovarian cancer, bladder cancer, endometrial cancer, uterine cancer, prostate cancer, lung cancer, esophageal cancer, head and neck cancer, colorectal cancer, kidney cancer, liver cancer, pancreatic cancer, stomach cancer, thyroid cancer, and melanoma.

96. The method of any one of claims 93-95, wherein the cancer is a cyclin D mediated cancer.

97. The method of any one of claims 93-96, wherein the cancer is characterized by amplification or overexpression of cyclin D (CCND), CDK4, and/or CDK6.

98. The method of any one of claims 93-97, wherein the cancer is characterized by primary or acquired resistance to treatment with a CDK4 and/or CDK6 inhibitor, or to endocrine therapy.
